(19) United States
(12) Patent Application Publication
(10) Pub. No.: US 2024/0215281 A1
(43) Pub. Date: Jun. 27, 2024

(54) COMPOUND, MATERIAL FOR ORGANIC ELECTROLUMINESCENT ELEMENT, ORGANIC ELECTROLUMINESCENT ELEMENT, AND ELECTRONIC DEVICE

(71) Applicant: IDEMITSU KOSAN CO.,LTD., Chiyoda-ku (JP)

(72) Inventors: Yusuke TAKAHASHI, Sodegaura-shi (JP); Tasuku HAKETA, Sodegaura-shi (JP); Shota TANAKA, Sodegaura-shi (JP); Takuto FUKAMI, Sodegaura-shi (JP); Tsukasa SAWATO, Sodegaura-shi (JP); Hiroaki ITOI, Sodegaura-shi (JP)

(73) Assignee: IDEMITSU KOSAN CO.,LTD., Chiyoda-ku (JP)

(21) Appl. No.: 18/546,490

(22) PCT Filed: Feb. 24, 2022
(86) PCT No.: PCT/JP2022/007685

§ 371 Date: Aug. 15, 2023

(30) Foreign Application Priority Data
Feb. 26, 2021 (JP) 2021-031230
Sep. 29, 2021 (JP) 2021-159354
Jan. 7, 2022 (WO) PCT/JP2022/000388

(51) Int. Cl. H10K 50/11; C07D 307/91; C07D 405/12; C07D 407/12; H10K 50/15; H10K 85/60

(52) U.S. Cl. CPC H10K 50/11; C07D 307/91; C07D 405/12; C07D 407/12; H10K 50/15; H10K 85/6574

(57) ABSTRACT

A compound represented by the following formula (1):

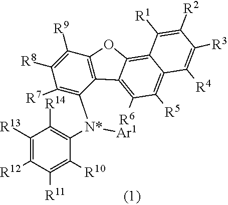

N*, R¹ to R¹⁴, and Ar¹ are as defined in the disclosure. An organic electroluminescent device, including an anode, a cathode, a light emitting layer intervening between the anode and the cathode, and an organic layer between the light emitting layer and the anode. The organic layer contains a compound represented by the following formula (2):

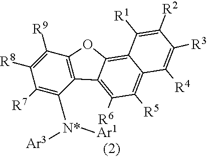

N*, R¹ to R⁹, Ar² and Ar³ are as defined in the disclosure. An electronic device including the organic electroluminescent device.

### TECHNICAL FIELD

The present invention relates to a compound, a material for organic electroluminescent devices, an organic electroluminescent device, and an electronic device including the organic luminescent device.

### BACKGROUND ART

In general, an organic electroluminescent device (which can be hereinafter referred to as an “organic EL device”) is constituted by an anode, a cathode, and an organic layer intervening between the anode and the cathode. In application of a voltage between both the electrodes, electrons from the cathode side and holes from the anode side are injected into a light emitting region, and the injected electrons and holes are recombined in the light emitting region to generate an excited state, which then returns to the ground state to emit light. Accordingly, development of a material that efficiently transports electrons or holes into the light emitting region, and promotes recombination of the electrons and holes is important for providing a high-performance organic EL device.

PTLs 1 to 8 describe compounds used for materials for organic electroluminescent devices.

### CITATION LIST

### Patent Literature

PTL 1: WO2020/111251A1
PTL 2: KR10-2019-0007789A
PTL 3: WO2020/231197A1
PTL 4: KR10-2018-0096458A
PTL 5: KR10-2016-0054374A
PTL 6: WO2020/149711A1
PTL 7: US2020/119282A1
PTL 8: WO2020/032574A1

### SUMMARY OF INVENTION

### Technical Problem

Various compounds for organic EL devices have been reported, but a compound that further enhances the capability of an organic EL device has been still demanded.

The present invention has been made for solving the problem, and an object thereof is to provide a compound that further improves the capability of an organic EL device, an organic EL device having a further improved device capability, and an electronic device including the organic EL device.

### Solution to Problem

As a result of the continued investigations by the present inventors on the capabilities of organic EL devices containing compounds for organic EL devices, it has been found that a monoamine, in which the 7-position of a naphthobenzofuran skeleton of a partial structure having a naphthobenzofuran skeleton bonds to the central nitrogen atom, or an aryl group or a heterocyclic group bonds thereto via a phenylene group, and further a partial structure having a specific cyclic structure bonds thereto, can provide an organic EL device having a further improved capability.

In one aspect, the present invention provides a compound represented by the following formula (1):

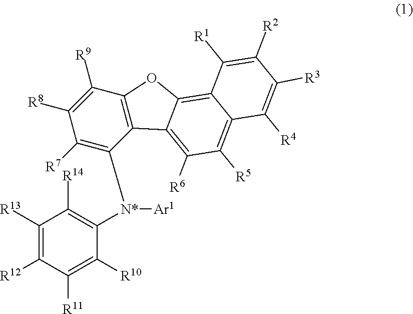

In the formula (1):
N* represents a central nitrogen atom;
R¹ to R⁹ each independently represent a hydrogen atom or a substituent A;
the substituent A is:
a halogen atom, a nitro group, a cyano group,
a substituted or unsubstituted alkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
a substituted or unsubstituted haloalkyl group having 1 to 50 carbon atoms,
a substituted or unsubstituted alkoxy group having 1 to 50 carbon atoms,
a substituted or unsubstituted haloalkoxy group having 1 to 50 carbon atoms,
a substituted or unsubstituted alkylthio group having 1 to 50 carbon atoms,
a substituted or unsubstituted aryloxy group having 6 to 50 ring carbon atoms,
a substituted or unsubstituted arylthio group having 6 to 50 ring carbon atoms,
a substituted or unsubstituted aralkyl group having 7 to 50 carbon atoms, or
a mono, di or tri-substituted silyl group having a substituent selected from a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, and a substituted or unsubstituted heterocyclic group having 5 to 50 ring atoms;
one of R¹⁰ to R¹⁴ is selected from a substituted or unsubstituted aryl group having 6 to 30 ring carbon atoms, and a substituted or unsubstituted heterocyclic group having 5 to 30 ring atoms; in the case where one of R¹⁰ to R¹⁴ is an aryl group, a ring constituting the aryl group is a 6-membered ring alone; R¹⁰ to R¹⁴ other than the one each are independently a hydrogen atom or the substituent A;
Ar¹ is represented by any of the following formulae (1-a) to (1-f):

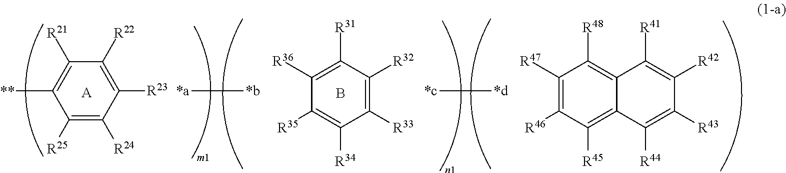

wherein:
* indicates a bonding position to the central nitrogen atom N*;
R²¹ to R²⁵, and R³¹ to R³⁶ each independently represent a hydrogen atom or the substituent A;
R⁴¹ to R⁴⁸ each independently represent a hydrogen atom, a phenyl group or the substituent A;
m1 represents 0, 1 or 2;
n1 represents 0, 1 or 2;
m1+n1 is 0, 1 or 2, provided that:
when m1 and n1 are 0, one selected from R¹ to R⁴⁸ is a single bond bonding to the central nitrogen atom N*,
when m1 is 0 and n1 is 1 or 2, one selected from R³¹ to R³⁶ is a single bond bonding to the central nitrogen atom N*, and another one selected from R³¹ to R³⁶ is a single bond bonding to *c, and one selected from R⁴¹ to R⁴⁸ is a single bond bonding to *d;
when n1 is 0 and m1 is 1 or 2, *a and *d bond together, one selected from R²¹ to R²⁵ is a single bond bonding to *a, and one selected from R⁴¹ to R⁴⁸ is a single bond bonding to *d;
when m1 and n1 are 1, one selected from R²¹ to R²⁵ is a single bond bonding to *a, one selected from R³¹ to R³⁶ is a single bond bonding to *b, another one selected from R³¹ to R³⁶ is a single bond bonding to *c, and one selected from R⁴¹ to R⁴⁸ is a single bond bonding to *d;
R²¹ to R²⁵ not being a single bond, R³¹ to R³⁶ not being a single bond, and R⁴¹ to R⁴⁸ not being a single bond do not bond to each other, and therefore do not form a cyclic structure;

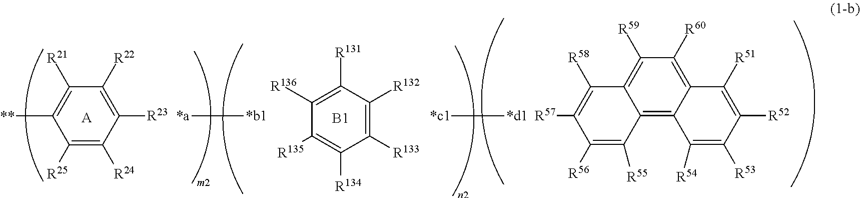

wherein:
* indicates a bonding position bonding to the central nitrogen atom N*;
R²¹ to R²⁵, and R¹³¹ to R¹³⁶ each independently represent a hydrogen atom or the substituent A;
R⁵¹ to R⁶⁰ each independently represent a hydrogen atom, a phenyl group or the substituent A;
m2 represents 0, 1 or 2;
n2 represents 0, 1 or 2;
m2+n2 is 0, 1 or 2, provided that:
when m2 and n2 are 0, one selected from R⁵¹ to R⁶⁰ is a single bond bonding to the central nitrogen atom N*;
when m2 is 0 and n2 is 1 or 2, one selected from R¹³¹ to R¹³⁶ is a single bond bonding to the central nitrogen atom N*, another one selected from R¹³¹ to R¹³⁶ is a single bond bonding to *c1, and one selected from R⁵¹ to R⁶⁰ is a single bond bonding to *d1;
when n2 is 0 and m2 is 1 or 2, *a and *d1 bond together, one selected from R²¹ to R²⁵ is a single bond bonding to *a, and one selected from R⁵¹ to R⁶⁰ is a single bond bonding *d1;
when m2 and n2 are 1, one selected from R²¹ to R²⁵ is a single bond bonding to *a, one selected from R¹³¹ to R¹³⁶ is a single bond bonding to *b1, another one selected from R¹³¹ to R¹³⁶ is a single bond bonding to *c1, and one selected from R⁵¹ to R⁶⁰ is a single bond bonding to *d1;
R²¹ to R²⁵ not being a single bond, R¹³¹ to R¹³⁶ not being a single bond, and R⁵¹ to R⁶⁰ not being a single bond do not bond to each other and therefore do not form a cyclic structure;

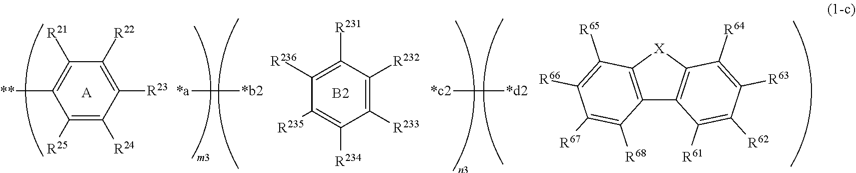

wherein:
* indicates a bonding position bonding to the central nitrogen atom N*;
X represents an oxygen atom, a sulfur atom, or NR^{a};
R^{a} represents a hydrogen atom, a substituted or unsubstituted alkyl group having 1 to 50 carbon atoms, or a substituted or unsubstituted aryl group having 6 to 50 ring carbon atoms;
R²¹ to R²⁵, and R²³¹ to R²³⁶ each independently represent a hydrogen atom or the substituent A;
R⁶¹ to R⁶⁸ each independently represent a hydrogen atom, a phenyl group, or the substituent A;
m3 represents 0, 1 or 2;
n3 represents 0, 1 or 2;
m3+n3 is 0, 1 or 2, provided that:
when m3 and n3 are 0, one selected from R⁶¹ to R⁶⁸ is a single bond bonding to the central nitrogen atom N*,
when m3 is 0 and n3 is 1 or 2, one selected from R²³¹ to R²³⁶ is a single bond bonding to the central nitrogen atom N*, another one selected from R²³¹ to R²³⁶ is a single bond bonding to *c2, and one selected from R⁶¹ to R⁶⁸ is a single bond bonding to *d2;
when n3 is 0 and m3 is 1 or 2, *a and *d2 bond together, one selected from R²¹ to R²⁵ is a single bond bonding to *a, and one selected from R⁶¹ to R⁶⁸ is a single bond bonding to *d2;
when m3 and n3 are 1, one selected from R²¹ to R²⁵ is a single bond bonding to *a, one selected from R²³¹ to R²³⁶ is a single bond bonding to *b2, another one selected from R²³¹ to R²³⁶ is a single bond bonding to *c2, and one selected from R⁶¹ to R⁶⁸ is a single bond bonding to *d2;
R²¹ to R²⁵ not being a single bond, and R²³¹ to R²³⁶ not being a single bond do not bond to each other and therefore do not form a cyclic structure;
one or more adjacent pairs of R⁶¹ to R⁶⁸ not being a single bond may or may not bond to each other to form a substituted or unsubstituted benzene ring;

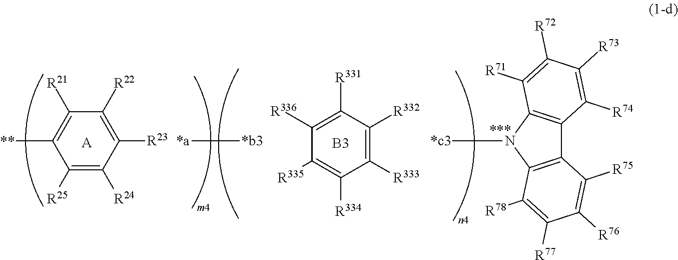

wherein:
* indicates a bonding position bonding to the central nitrogen atom N*;
R²¹ to R²⁵, and R³³¹ to R³³⁶ each independently represent a hydrogen atom or the substituent A;
R⁷¹ to R⁷⁸ each independently represent a hydrogen atom, a phenyl group or the substituent A;
m4 represents 0, 1 or 2;
n4 represents 0, 1 or 2;
m4+n4 is 1 or 2, provided that:
when m4 is 0 and n4 is 1 or 2, one selected from R³³¹ to R³³⁶ is a single bond bonding the central nitrogen atom N*, and another one selected from R³³¹ to R³³⁶ is a single bond bonding to *c3;
when n4 is 0 and m4 is 1 or 2, one selected from R²¹ to R²⁵ is a single bond bonding to the nitrogen atom N***;
when m4 and n4 are 1, one selected from R²¹ to R²⁵ is a single bond bonding to *a, one selected from R³³¹ to R³³⁶ is a single bond bonding to *b3, and another one selected from R³³¹ to R³³⁶ is a single bond bonding to *c3;
R²¹ to R²⁵ not being a single bond, R³³¹ to R³³⁵ not being a single bond, and R⁷¹ to R⁷⁸ do not bond to each other and therefore do not form a cyclic structure;

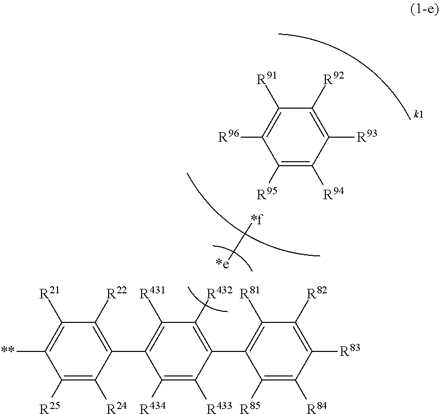

wherein:
** indicates a bonding position bonding to the central nitrogen atom N*,
R²¹, R²², R²⁴, R²⁵, R⁴³¹ to R⁴³⁴, R⁸¹ to R⁸⁵, and R⁹¹ to R⁹⁶ each independently represent a hydrogen atom or the substituent A;
k1 represents 0 or 1, provided that:
when k1 is 1, R⁴³² is a single bond bonding to *e, and one selected from R⁹¹ to R⁹⁶ is a single bond bonding to *f;
R⁹¹ to R⁹⁶ not being a single bond, R⁴³² not being a single bond, R⁴³¹, R⁴³³, R⁴³⁴, R²¹, R²², R²⁴, R²⁵, and R⁸¹ to R⁸³ do not bond to each other and therefore do not form a cyclic structure;

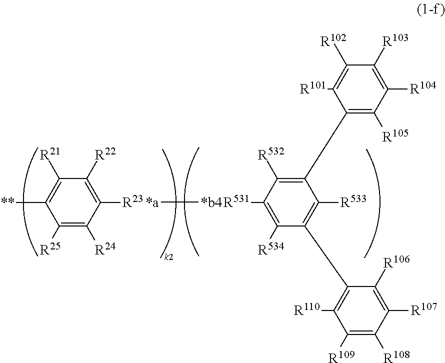

wherein:
* indicates a bonding position boning to the central nitrogen atom N*;
R²¹ to R²⁵, R⁵³¹ to R⁵³⁴, and R¹⁰¹ to R¹¹⁰ each independently represent a hydrogen atom or the substituent A;
k2 represents 0 or 1, provide that:
when k2 is 0, *a indicates a bonding position bonding to the central nitrogen atom N*,
when k2 is 1, one selected from R²¹ to R²⁵ is a single bond bonding to *a, and one selected from R⁵³¹ to R⁵³⁴ is a single bond bonding to *b4;
R²¹ to R²⁵ not being a single bond, R⁵³¹ to R⁵³⁴ not being a single bond, R¹⁰¹ to R¹⁰⁵, and R¹⁰⁶ to R¹¹⁰ do not bond to each other and therefore do not form a cyclic structure.

In another aspect, the present invention provides a material for an organic EL device containing the compound represented by the formula (1).

In still another aspect, the present invention provides an organic electroluminescent device including an anode, a cathode, and a light emitting layer intervening between the anode and the cathode, and having an organic layer between the light emitting layer and the anode, wherein the organic layer contains a compound represented by the following formula (2):

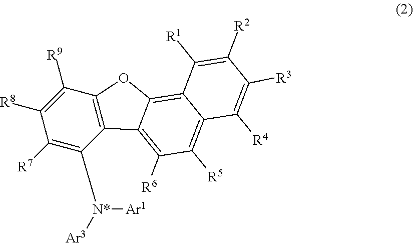

In the formula (2):
N* represents a central nitrogen atom:
R¹ to R⁹ each independently represent a hydrogen atom or a substituent A;
the substituent A is:
a halogen atom, a nitro group, a cyano group,
a substituted or unsubstituted alkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
a substituted or unsubstituted haloalkyl group having 1 to 50 carbon atoms,
a substituted or unsubstituted alkoxy group having 1 to 50 carbon atoms,
a substituted or unsubstituted haloalkoxy group having 1 to 50 carbon atoms,
a substituted or unsubstituted alkylthio group having 1 to 50 carbon atoms,
a substituted or unsubstituted aryloxy group having 6 to 50 ring carbon atoms,
a substituted or unsubstituted arylthio group having 6 to 50 ring carbon atoms,
a substituted or unsubstituted aralkyl group having 7 to 50 carbon atoms, or
a mono, di or tri-substituted silyl group having a substituent selected from a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, and a substituted or unsubstituted heterocyclic group having 5 to 50 ring atoms;
Ar² and Ar³ each independently represent a substituted or unsubstituted aryl group having 6 to 30 ring carbon atoms, or a substituted or unsubstituted heterocyclic group having 5 to 30 ring atoms.

In a further aspect, the present invention provides an electronic device including the organic electroluminescent device.

### Advantageous Effects of Invention

An organic EL device containing the compound represented by the formula (1) shows an improved device capability.

### BRIEF DESCRIPTION OF DRAWINGS

FIG. 1 is a schematic illustration showing an example of a layer configuration of an organic EL device according to one aspect of the present invention.

FIG. 2 is a schematic illustration showing another example of the layer configuration of the organic EL device according to one aspect of the present invention.

### DESCRIPTION OF EMBODIMENTS

### Definitions

In the description herein, the hydrogen atom encompasses isotopes thereof having different numbers of neutrons, i.e., a light hydrogen atom (protium), a heavy hydrogen atom (deuterium), and tritium.

In the description herein, the bonding site where the symbol, such as “R”, or “D” representing a deuterium atom is not shown is assumed to have a hydrogen atom, i.e., a protium atom, a deuterium atom, or a tritium atom, bonded thereto.

In the description herein, the number of ring carbon atoms shows the number of carbon atoms among the atoms constituting the ring itself of a compound having a structure including atoms bonded to form a ring (such as a monocyclic compound, a condensed ring compound, a bridged compound, a carbocyclic compound, and a heterocyclic compound). In the case where the ring is substituted by a substituent, the carbon atom contained in the substituent is not included in the number of ring carbon atoms. The same definition is applied to the “number of ring carbon atoms” described hereinafter unless otherwise indicated. For example, a benzene ring has 6 ring carbon atoms, a naphthalene ring has 10 ring carbon atoms, a pyridine ring has 5 ring carbon atoms, and a furan ring has 4 ring carbon atoms. For example, 9,9-diphenylfluorenyl group has 13 ring carbon atoms, and 9,9′-spirobifluorenyl group has 25 ring carbon atoms.

In the case where a benzene ring has, for example, an alkyl group substituted thereon as a substituent, the number of carbon atoms of the alkyl group is not included in the number of ring carbon atoms of the benzene ring. Accordingly, a benzene ring having an alkyl group substituted thereon has 6 ring carbon atoms. In the case where a naphthalene ring has, for example, an alkyl group substituted thereon as a substituent, the number of carbon atoms of the alkyl group is not included in the number of ring carbon atoms of the naphthalene ring. Accordingly, a naphthalene ring having an alkyl group substituted thereon has 10 ring carbon atoms.

In the description herein, the number of ring atoms shows the number of atoms constituting the ring itself of a compound having a structure including atoms bonded to form a ring (such as a monocyclic ring, a condensed ring, and a set of rings) (such as a monocyclic compound, a condensed ring compound, a bridged compound, a carbocyclic compound, and a heterocyclic compound). The atom that does not constitute the ring (such as a hydrogen atom terminating the bond of the atom constituting the ring) and, in the case where the ring is substituted by a substituent, the atom contained in the substituent are not included in the number of ring atoms. The same definition is applied to the “number of ring atoms” described hereinafter unless otherwise indicated. For example, a pyridine ring has 6 ring atoms, a quinazoline ring has 10 ring atoms, and a furan ring has 5 ring atoms. For example, the number of hydrogen atoms bonded to a pyridine ring or atoms constituting a substituent is not included in the number of ring atoms of the pyridine ring. Accordingly, a pyridine ring having a hydrogen atom or a substituent bonded thereto has 6 ring atoms. For example, the number of hydrogen atoms bonded to carbon atoms of a quinazoline ring or atoms constituting a substituent is not included in the number of ring atoms of the quinazoline ring. Accordingly, a quinazoline ring having a hydrogen atom or a substituent bonded thereto has 10 ring atoms.

In the description herein, the expression “having XX to YY carbon atoms” in the expression “substituted or unsubstituted ZZ group having XX to YY carbon atoms” means the number of carbon atoms of the unsubstituted ZZ group, and, in the case where the ZZ group is substituted, the number of carbon atoms of the substituent is not included. Herein, “YY” is larger than “XX”, “XX” represents an integer of 1 or more, and “YY” represents an integer of 2 or more.

In the description herein, the expression “having XX to YY atoms” in the expression “substituted or unsubstituted ZZ group having XX to YY atoms” means the number of atoms of the unsubstituted ZZ group, and, in the case where the ZZ group is substituted, the number of atoms of the substituent is not included. Herein, “YY” is larger than “XX”, “XX” represents an integer of 1 or more, and “YY” represents an integer of 2 or more.

In the description herein, an unsubstituted ZZ group means the case where the “substituted or unsubstituted ZZ group” is an “unsubstituted ZZ group”, and a substituted ZZ group means the case where the “substituted or unsubstituted ZZ group” is a “substituted ZZ group”.

In the description herein, the expression “unsubstituted” in the expression “substituted or unsubstituted ZZ group” means that hydrogen atoms in the ZZ group are not substituted by a substituent. The hydrogen atoms in the “unsubstituted ZZ group” each are a protium atom, a deuterium atom, or a tritium atom.

In the description herein, the expression “substituted” in the expression “substituted or unsubstituted ZZ group” means that one or more hydrogen atoms in the ZZ group is substituted by a substituent. The expression “substituted” in the expression “BB group substituted by an AA group” similarly means that one or more hydrogen atoms in the BB group is substituted by the AA group.

### Substituents in Description

The substituents described in the description herein will be explained.

In the description herein, the number of ring carbon atoms of the “unsubstituted aryl group” is 6 to 50, preferably 6 to 30, and more preferably 6 to 18, unless otherwise indicated in the description.

In the description herein, the number of ring atoms of the “unsubstituted heterocyclic group” is 5 to 50, preferably 5 to 30, and more preferably 5 to 18, unless otherwise indicated in the description.

In the description herein, the number of carbon atoms of the “unsubstituted alkyl group” is 1 to 50, preferably 1 to 20, and more preferably 1 to 6, unless otherwise indicated in the description.

In the description herein, the number of carbon atoms of the “unsubstituted alkenyl group” is 2 to 50, preferably 2 to 20, and more preferably 2 to 6, unless otherwise indicated in the description.

In the description herein, the number of carbon atoms of the “unsubstituted alkynyl group” is 2 to 50, preferably 2 to 20, and more preferably 2 to 6, unless otherwise indicated in the description.

In the description herein, the number of ring carbon atoms of the “unsubstituted cycloalkyl group” is 3 to 50, preferably 3 to 20, and more preferably 3 to 6, unless otherwise indicated in the description.

In the description herein, the number of ring carbon atoms ofthe “unsubstituted arylene group” is 6 to 50, preferably 6 to 30, and more preferably 6 to 18, unless otherwise indicated in the description.

In the description herein, the number of ring atoms of the “unsubstituted divalent heterocyclic group” is 5 to 50, preferably 5 to 30, and more preferably 5 to 18, unless otherwise indicated in the description.

In the description herein, the number of carbon atoms of the “unsubstituted alkylene group” is 1 to 50, preferably 1 to 20, and more preferably 1 to 6, unless otherwise indicated in the description.

### Substituted or Unsubstituted Aryl Group

In the description herein, specific examples (set of specific examples G1) of the “substituted or unsubstituted aryl group” include the unsubstituted aryl groups (set of specific examples G1A) and the substituted aryl groups (set of specific examples G1B) shown below. (Herein, the unsubstituted aryl group means the case where the “substituted or unsubstituted aryl group” is an “unsubstituted aryl group”, and the substituted aryl group means the case where the “substituted or unsubstituted aryl group” is a “substituted aryl group”.) In the description herein, the simple expression “aryl group” encompasses both the “unsubstituted aryl group” and the “substituted aryl group”.

The “substituted aryl group” means a group formed by substituting one or more hydrogen atoms of the “unsubstituted aryl group” by a substituent. Examples of the “substituted aryl group” include groups formed by one or more hydrogen atoms of each of the “unsubstituted aryl groups” in the set of specific examples G1A by a substituent, and the examples of the substituted aryl groups in the set of specific examples GIB. The examples of the “unsubstituted aryl group” and the examples of the “substituted aryl group” enumerated herein are mere examples, and the “substituted aryl group” in the description herein encompasses groups formed by substituting a hydrogen atom bonded to the carbon atom of the aryl group itself of each of the “substituted aryl groups” in the set of specific examples G1B by a substituent, and groups formed by substituting a hydrogen atom of the substituent of each of the “substituted aryl groups” in the set of specific examples G1B by a substituent.

Unsubstituted Aryl Group (Set of Specific Examples G1A):
a phenyl group,
a p-biphenyl group,
a m-biphenyl group,
an o-biphenyl group,
a p-terphenyl-4-yl group,
a p-terphenyl-3-yl group,
a p-terphenyl-2-yl group,
a m-terphenyl-4-yl group,
a m-terphenyl-3-yl group,
a m-terphenyl-2-yl group,
an o-terphenyl-4-yl group,
an o-terphenyl-3-yl group,
an o-terphenyl-2-yl group,
a 1-naphthyl group,
a 2-naphthyl group,
an anthryl group,
a benzanthryl group,
a phenanthryl group,
a benzophenanthryl group,
a phenarenyl group,
a pyrenyl group,
a chrysenyl group,
a benzochrysenyl group,
a triphenylenyl group,
a benzotriphenylenyl group,
a tetracenyl group,
a pentacenyl group,
a fluorenyl group,
a 9,9′-spirobifluorenyl group,
a benzofluorenyl group,
a dibenzofluorenyl group,
a fluoranthenyl group,
a benzofluoranthenyl group,
a perylenyl group, and
monovalent aryl groups derived by removing one hydrogen atom from each of the ring structures represented by the following general formulae (TEMP-1) to (TEMP-15):

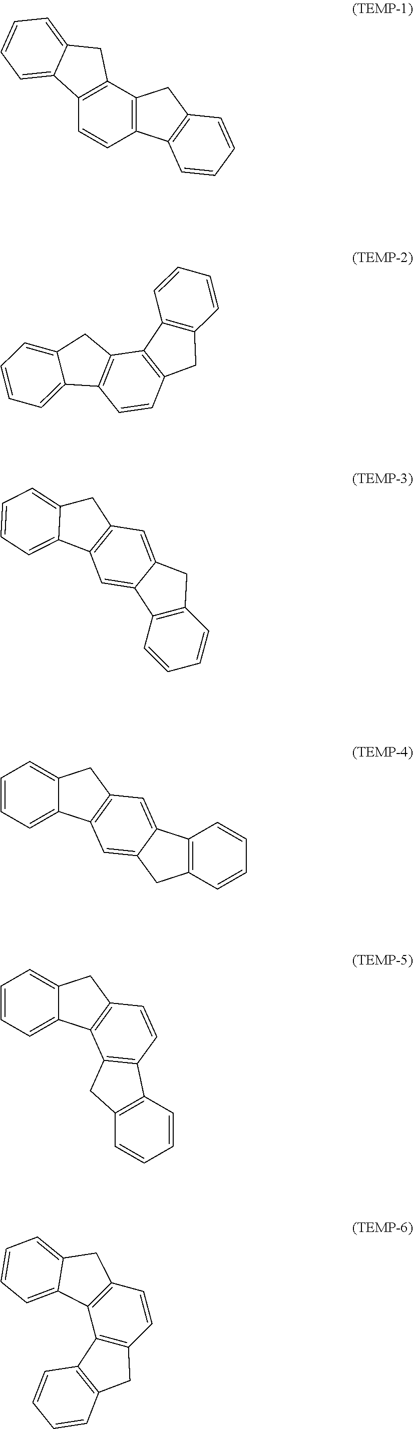

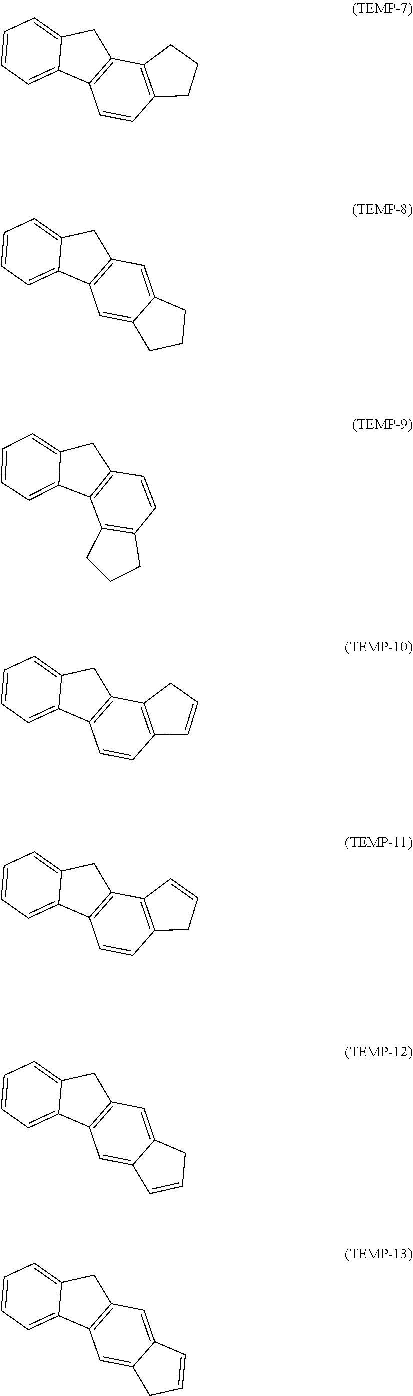

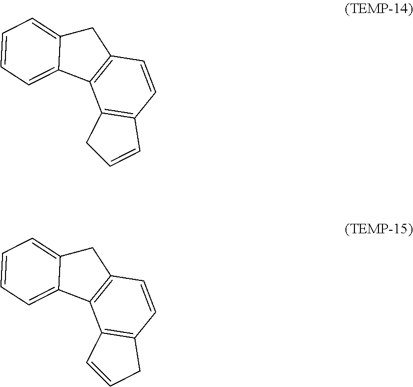

Substituted Aryl Group (Set of Specific Examples G1B):
an o-tolyl group,
a m-tolyl group,
a p-tolyl group,
a p-xylyl group,
a m-xylyl group,
an o-xylyl group,
a p-isopropylphenyl group,
a m-isopropylphenyl group,
an o-isopropylphenyl group,
a p-t-butylphenyl group,
a m-t-butylphenyl group,
an o-t-butylphenyl group,
a 3,4,5-trimethylphenyl group,
a 9,9-dimethylfluorenyl group,
a 9,9-diphenylfluorenyl group,
a 9,9-bis(4-methylphenyl)fluorenyl group,
a 9,9-bis(4-isopropylphenyl)fluorenyl group,
a 9,9-bis(4-t-butylphenyl)fluorenyl group,
a cyanophenyl group,
a triphenylsilylphenyl group,
a trimethylsilylphenyl group,
a phenylnaphthyl group,
a naphthylphenyl group, and
groups formed by substituting one or more hydrogen atoms of each of monovalent aryl
groups derived from the ring structures represented by the general formulae (TEMP-1) to
(TEMP-15) by a substituent.

### Substituted or Unsubstituted Heterocyclic Group

In the description herein, the “heterocyclic group” means a cyclic group containing at least one hetero atom in the ring atoms. Specific examples of the hetero atom include a nitrogen atom, an oxygen atom, a sulfur atom, a silicon atom, a phosphorus atom, and a boron atom.

In the description herein, the “heterocyclic group” is a monocyclic group or a condensed ring group.

In the description herein, the “heterocyclic group” is an aromatic heterocyclic group or a non-aromatic heterocyclic group.

In the description herein, specific examples (set of specific examples G2) of the “substituted or unsubstituted heterocyclic group” include the unsubstituted heterocyclic groups (set of specific examples G2A) and the substituted heterocyclic groups (set of specific examples G2B) shown below. (Herein, the unsubstituted heterocyclic group means the case where the “substituted or unsubstituted heterocyclic group” is an “unsubstituted heterocyclic group”, and the substituted heterocyclic group means the case where the “substituted or unsubstituted heterocyclic group” is a “substituted heterocyclic group”.) In the description herein, the simple expression “heterocyclic group” encompasses both the “unsubstituted heterocyclic group” and the “substituted heterocyclic group”.

The “substituted heterocyclic group” means a group formed by substituting one or more hydrogen atoms of the “unsubstituted heterocyclic group” by a substituent. Specific examples of the “substituted heterocyclic group” include groups formed by substituting a hydrogen atom of each of the “unsubstituted heterocyclic groups” in the set of specific examples G2A by a substituent, and the examples of the substituted heterocyclic groups in the set of specific examples G2B. The examples of the “unsubstituted heterocyclic group” and the examples of the “substituted heterocyclic group” enumerated herein are mere examples, and the “substituted heterocyclic group” in the description herein encompasses groups formed by substituting a hydrogen atom bonded to the ring atom of the heterocyclic group itself of each of the “substituted heterocyclic groups” in the set of specific examples G2B by a substituent, and groups formed by substituting a hydrogen atom of the substituent of each of the “substituted heterocyclic groups” in the set of specific examples G2B by a substituent.

The set of specific examples G2A includes, for example, the unsubstituted heterocyclic group containing a nitrogen atom (set of specific examples G2A1), the unsubstituted heterocyclic group containing an oxygen atom (set of specific examples G2A2), the unsubstituted heterocyclic group containing a sulfur atom (set of specific examples G2A3), and monovalent heterocyclic groups derived by removing one hydrogen atom from each of the ring structures represented by the following general formulae (TEMP-16) to (TEMP-33) (set of specific examples G2A4).

The set of specific examples G2B includes, for example, the substituted heterocyclic groups containing a nitrogen atom (set of specific examples G2B1), the substituted heterocyclic groups containing an oxygen atom (set of specific examples G2B2), the substituted heterocyclic groups containing a sulfur atom (set of specific examples G2B3), and groups formed by substituting one or more hydrogen atoms of each of monovalent heterocyclic groups derived from the ring structures represented by the following general formulae (TEMP-16) to (TEMP-33) by a substituent (set of specific examples G2B4).

Unsubstituted Heterocyclic Group containing Nitrogen Atom (Set of Specific Examples G2A1):
a pyrrolyl group,
an imidazolyl group,
a pyrazolyl group,
a triazolyl group,
a tetrazolyl group,
an oxazolyl group,
an isoxazolyl group,
an oxadiazolyl group,
a thiazolyl group,
an isothiazolyl group,
a thiadiazolyl group,
a pyridyl group,
a pyridazinyl group,
a pyrimidinyl group,
a pyrazinyl group,
a triazinyl group,
an indolyl group,
an isoindolyl group,
an indolizinyl group,
a quinolizinyl group,
a quinolyl group,
an isoquinolyl group,
a cinnolinyl group,
a phthalazinyl group,
a quinazolinyl group,
a quinoxalinyl group,
a benzimidazolyl group,
an indazolyl group,
a phenanthrolinyl group,
a phenanthridinyl group,
an acridinyl group,
a phenazinyl group,
a carbazolyl group,
a benzocarbazolyl group,
a morpholino group,
a phenoxazinyl group,
a phenothiazinyl group,
an azacarbazolyl group, and
a diazacarbazolyl group.

Unsubstituted Heterocyclic Group containing Oxygen Atom (Set of Specific Examples G2A2):
a fuiryl group,
an oxazolyl group,
an isoxazolyl group,
an oxadiazolyl group,
a xanthenyl group,
a benzofuranyl group,
an isobenzofuranyl group,
a dibenzofuranyl group,
a naphthobenzofuranyl group,
a benzoxazolyl group,
a benzisoxazolyl group,
a phenoxazinyl group,
a morpholino group,
a dinaphthofuranyl group,
an azadibenzofuranyl group,
a diazadibenzofuranyl group,
an azanaphthobenzofuranyl group, and
a diazanaphthobenzofuranyl group.

Unsubstituted Heterocyclic Group containing Sulfur Atom (Set of Specific Examples G2A3):
a thienyl group,
a thiazolyl group,
an isothiazolyl group,
a thiadiazolyl group,
a benzothiophenyl group (benzothienyl group),
an isobenzothiophenyl group (isobenzothienyl group),
a dibenzothiophenyl group (dibenzothienyl group),
a naphthobenzothiophenyl group (naphthobenzothienyl group),
a benzothiazolyl group,
a benzisothiazolyl group,
a phenothiazinyl group,
a dinaphthothiophenyl group (dinaphthothienyl group),
an azadibenzothiophenyl group (azadibenzothienyl group),
a diazadibenzothiophenyl group (diazadibenzothienyl group),
an azanaphthobenzothiophenyl group (azanaphthobenzothienyl group), and
a diazanaphthobenzothiophenyl group (diazanaphthobenzothienyl group).

Monovalent Heterocyclic Group derived by removing One Hydrogen Atom from Ring Structures represented by General Formulae (TEMP-16) to (TEMP-33) (Set of Specific Examples G2A4)

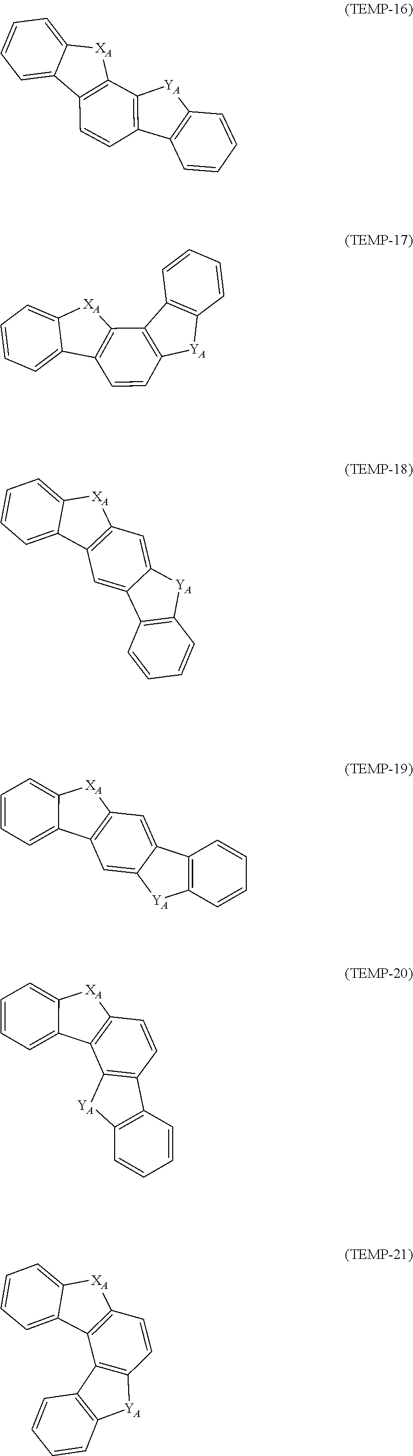

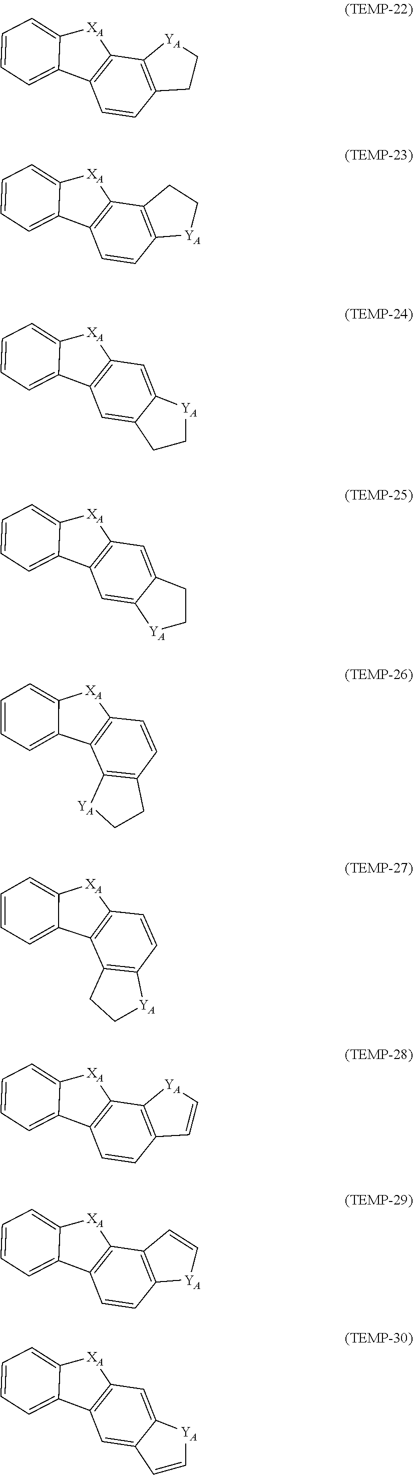

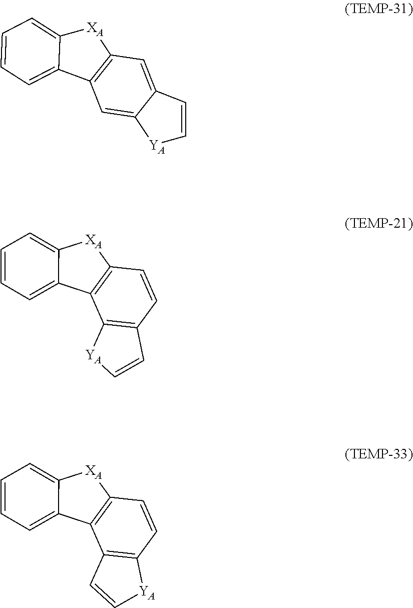

In the general formulae (TEMP-16) to (TEMP-33), X_{A} and Y_{A} each independently represent an oxygen atom, a sulfur atom, NH, or CH₂, provided that at least one of X_{A} and Y_{A} represents an oxygen atom, a sulfur atom, or NH.

In the general formulae (TEMP-16) to (TEMP-33), in the case where at least one of X_{A} and Y_{A} represents NH or CH₂, the monovalent heterocyclic groups derived from the ring structures represented by the general formulae (TEMP-16) to (TEMP-33) include monovalent groups formed by removing one hydrogen atom from the NH or CH₂.

Substituted Heterocyclic Group containing Nitrogen Atom (Set of Specific Examples G2B1):
a (9-phenyl)carbazolyl group,
a (9-biphenylyl)carbazolyl group,
a (9-phenyl)phenylcarbazolyl group,
a (9-naphthyl)carbazolyl group,
a diphenylcarbazol-9-yl group,
a phenylcarbazol-9-yl group,
a methylbenzimidazolyl group,
an ethylbenzimidazolyl group,
a phenyltriazinyl group,
a biphenyltriazinyl group,
a diphenyltriazinyl group,
a phenylquinazolinyl group, and
a biphenylquinazolinyl group.

Substituted Heterocyclic Group containing Oxygen Atom (Set of Specific Examples G2B2):
a phenyldibenzofuranyl group,
a methyldibenzofuranyl group,
a t-butyldibenzofiranyl group, and
a monovalent residual group of spiro[9H-xanthene-9,9′-[9H]fluorene].

Substituted Heterocyclic Group containing Sulfur Atom (Set of Specific Examples G2B3):
a phenyldibenzothiophenyl group,
a methyldibenzothiophenyl group,
a t-butyldibenzothiophenyl group, and
a monovalent residual group of spiro[9H-thioxanthene-9,9′-[9H]fluorene].

Group formed by substituting one or more Hydrogen Atoms of Monovalent Heterocyclic Group derived from Ring Structures represented by General Formulae (TEMP-16) to (TEMP-33) by Substituent (Set of Specific Examples G2B4)

The “one or more hydrogen atoms of the monovalent heterocyclic group” means one or more hydrogen atoms selected from the hydrogen atom bonded to the ring carbon atom of the monovalent heterocyclic group, the hydrogen atom bonded to the nitrogen atom in the case where at least one of X_{A} and Y_{A} represents NH, and the hydrogen atom of the methylene group in the case where one of X_{A} and Y_{A} represents CH₂.

### Substituted or Unsubstituted Alkyl Group

In the description herein, specific examples (set of specific examples G3) of the “substituted or unsubstituted alkyl group” include the unsubstituted alkyl groups (set of specific examples G3A) and the substituted alkyl groups (set of specific examples G3B) shown below. (Herein, the unsubstituted alkyl group means the case where the “substituted or unsubstituted alkyl group” is an “unsubstituted alkyl group”, and the substituted alkyl group means the case where the “substituted or unsubstituted alkyl group” is a “substituted alkyl group”.) In the description herein, the simple expression “alkyl group” encompasses both the “unsubstituted alkyl group” and the “substituted alkyl group”.

The “substituted alkyl group” means a group formed by substituting one or more hydrogen atoms of the “unsubstituted alkyl group” by a substituent. Specific examples of the “substituted alkyl group” include groups formed by substituting one or more hydrogen atoms of each of the “unsubstituted alkyl groups” (set of specific examples G3A) shown below by a substituent, and the examples of the substituted alkyl groups (set of specific examples G3B). In the description herein, the alkyl group in the “unsubstituted alkyl group” means a chain-like alkyl group. Accordingly, the “unsubstituted alkyl group” encompasses an “unsubstituted linear alkyl group” and an “unsubstituted branched alkyl group”. The examples of the “unsubstituted alkyl group” and the examples of the “substituted alkyl group” enumerated herein are mere examples, and the “substituted alkyl group” in the description herein encompasses groups formed by substituting a hydrogen atom of the alkyl group itself of each of the “substituted alkyl groups” in the set of specific examples G3B by a substituent, and groups formed by substituting a hydrogen atom of the substituent of each of the “substituted alkyl groups” in the set of specific examples G3B by a substituent.

Unsubstituted Alkyl Group (Set of Specific Examples G3A):
a methyl group,
an ethyl group,
a n-propyl group,
an isopropyl group,
a n-butyl group,
an isobutyl group,
a s-butyl group, and
a t-butyl group.

Substituted Alkyl Group (Set of Specific Examples G3B):
a heptafluoropropyl group (including isomers),
a pentafluoroethyl group,
a 2,2,2-trifluoroethyl group, and
a trifluoromethyl group.

### Substituted or Unsubstituted Alkenyl Group

In the description herein, specific examples (set of specific examples G4) of the “substituted or unsubstituted alkenyl group” include the unsubstituted alkenyl groups (set of specific examples G4A) and the substituted alkenyl groups (set of specific examples G4B) shown below. (Herein, the unsubstituted alkenyl group means the case where the “substituted or unsubstituted alkenyl group” is an “unsubstituted alkenyl group”, and the substituted alkenyl group means the case where the “substituted or unsubstituted alkenyl group” is a “substituted alkenyl group”.) In the description herein, the simple expression “alkenyl group” encompasses both the “unsubstituted alkenyl group” and the “substituted alkenyl group”.

The “substituted alkenyl group” means a group formed by substituting one or more hydrogen atoms of the “unsubstituted alkenyl group” by a substituent. Specific examples of the “substituted alkenyl group” include the “unsubstituted alkenyl groups” shown below (set of specific examples G4A) that each have a substituent, and the examples of the substituted alkenyl groups (set of specific examples G4B). The examples of the “unsubstituted alkenyl group” and the examples of the “substituted alkenyl group” enumerated herein are mere examples, and the “substituted alkenyl group” in the description herein encompasses groups formed by substituting a hydrogen atom of the alkenyl group itself of each of the “substituted alkenyl groups” in the set of specific examples G4B by a substituent, and groups formed by substituting a hydrogen atom of the substituent of each of the “substituted alkenyl groups” in the set of specific examples G4B by a substituent.

Unsubstituted Alkenyl Group (Set of Specific Examples G4A):
a vinyl group,
an allyl group,
a 1-butenyl group,
a 2-butenyl group, and
a 3-butenyl group.

### Substituted Alkenyl Group (Set of Specific Examples G4B):

a 1,3-butanedienyl group,
a 1-methylvinyl group,
a 1-methylallyl group,
a 1,1-dimethylallyl group,
a 2-methylallyl group, and
a 1,2-dimethylallyl group.

### Substituted or Unsubstituted Alkynyl Group

In the description herein, specific examples (set of specific examples G5) of the “substituted or unsubstituted alkynyl group” include the unsubstituted alkynyl group (set of specific examples G5A) shown below. (Herein, the unsubstituted alkynyl group means the case where the “substituted or unsubstituted alkynyl group” is an “unsubstituted alkynyl group”.) In the description herein, the simple expression “alkynyl group” encompasses both the “unsubstituted alkynyl group” and the “substituted alkynyl group”.

The “substituted alkynyl group” means a group formed by substituting one or more hydrogen atoms of the “unsubstituted alkynyl group” by a substituent. Specific examples of the “substituted alkenyl group” include groups formed by substituting one or more hydrogen atoms of the “unsubstituted alkynyl group” shown below (set of specific examples G5A) by a substituent.

### Unsubstituted Alkynyl Group (Set of Specific Examples G5A):

an ethynyl group.

### Substituted or Unsubstituted Cycloalkyl Group

In the description herein, specific examples (set of specific examples G6) of the “substituted or unsubstituted cycloalkyl group” include the unsubstituted cycloalkyl groups (set of specific examples G6A) and the substituted cycloalkyl group (set of specific examples G6B) shown below. (Herein, the unsubstituted cycloalkyl group means the case where the “substituted or unsubstituted cycloalkyl group” is an “unsubstituted cycloalkyl group”, and the substituted cycloalkyl group means the case where the “substituted or unsubstituted cycloalkyl group” is a “substituted cycloalkyl group”.) In the description herein, the simple expression “cycloalkyl group” encompasses both the “unsubstituted cycloalkyl group” and the “substituted cycloalkyl group”.

The “substituted cycloalkyl group” means a group formed by substituting one or more hydrogen atoms of the “unsubstituted cycloalkyl group” by a substituent. Specific examples of the “substituted cycloalkyl group” include groups formed by substituting one or more hydrogen atoms of each of the “unsubstituted cycloalkyl groups” shown below (set of specific examples G6A) by a substituent, and the example of the substituted cycloalkyl group (set of specific examples G6B). The examples of the “unsubstituted cycloalkyl group” and the examples of the “substituted cycloalkyl group” enumerated herein are mere examples, and the “substituted cycloalkyl group” in the description herein encompasses groups formed by substituting one or more hydrogen atoms bonded to the carbon atoms of the cycloalkyl group itself of the “substituted cycloalkyl group” in the set of specific examples G6B by a substituent, and groups formed by substituting a hydrogen atom of the substituent of the “substituted cycloalkyl group” in the set of specific examples G6B by a substituent.

Unsubstituted Cycloalkyl Group (Set of Specific Examples G6A):
a cyclopropyl group,
a cyclobutyl group,
a cyclopentyl group,
a cyclohexyl group,
a 1-adamantyl group,
a 2-adamantyl group,
a 1-norbornyl group, and
a 2-norbornyl group.

Substituted Cycloalkyl Group (Set of Specific Examples G6B):
a 4-methylcyclohexyl group.
   Group Represented by —Si(R₉₀₁)(R₉₀₂)(R₉₀₃)

In the description herein, specific examples (set of specific examples G7) of the group represented by —Si(R₉₀₁)(R₉₀₂)(R₉₀₃) include:
Si(G1)(G1)(G1),
Si(G1)(G2)(G2),
Si(G1)(G1)(G2),
Si(G2)(G2)(G2),
Si(G3)(G3)(G3), and
Si(G6)(G6)(G6).

Herein,
G1 represents the “substituted or unsubstituted aryl group” described in the set of specific examples G1,
G2 represents the “substituted or unsubstituted heterocyclic group” described in the set of specific examples G2,
G3 represents the “substituted or unsubstituted alkyl group” described in the set of specific examples G3, and
G6 represents the “substituted or unsubstituted cycloalkyl group” described in the set of specific examples G6.

Plural groups represented by G1 in —Si(G1)(G1)(G1) are the same as or different from each other.

Plural groups represented by G2 in —Si(G1)(G2)(G2) are the same as or different from each other.

Plural groups represented by G1 in —Si(G1)(G1)(G2) are the same as or different from each other.

Plural groups represented by G2 in —Si(G2)(G2)(G2) are the same as or different from each other.

Plural groups represented by G3 in —Si(G3)(G3)(G3) are the same as or different from each other.

Plural groups represented by G6 in —Si(G6)(G6)(G6) are the same as or different from each other.

Group represented by —O—(R₉₀₄)

In the description herein, specific examples (set of specific examples G8) of the group represented by —O—(R₉₀₄) include:
—O(G1),
—O(G2),
—O(G3), and
—O(G6).

Herein,
G1 represents the “substituted or unsubstituted aryl group” described in the set of specific examples G1,
G2 represents the “substituted or unsubstituted heterocyclic group” described in the set of specific examples G2,
G3 represents the “substituted or unsubstituted alkyl group” described in the set of specific examples G3, and
G6 represents the “substituted or unsubstituted cycloalkyl group” described in the set of specific examples G6.
   Group represented by —S—(R₉₀₅)

In the description herein, specific examples (set of specific examples G9) of the group represented by —S—(R₉₀₅) include:
—S(G1),
—S(G2),
—S(G3), and
—S(G6).

Herein,
G1 represents the “substituted or unsubstituted aryl group” described in the set of specific examples G1,
G2 represents the “substituted or unsubstituted heterocyclic group” described in the set of specific examples G2,
G3 represents the “substituted or unsubstituted alkyl group” described in the set of specific examples G3, and
G6 represents the “substituted or unsubstituted cycloalkyl group” described in the set of specific examples G6.
   Group Represented by —N(R₉₀₆)(R₉₀₇)

In the description herein, specific examples (set of specific examples G10) of the group represented by —N(R₉₀₆)(R₉₀₇) include:
—N(G1)(G1),
—N(G2)(G2),
—N(G1)(G2),
—N(G3)(G3), and
—N(G6)(G6).
G1 represents the “substituted or unsubstituted aryl group” described in the set of specific examples G1,
G2 represents the “substituted or unsubstituted heterocyclic group” described in the set of specific examples G2,
G3 represents the “substituted or unsubstituted alkyl group” described in the set of specific examples G3, and
G6 represents the “substituted or unsubstituted cycloalkyl group” described in the set of specific examples G6.

Plural groups represented by G1 in —N(G1)(G1) are the same as or different from each other.

Plural groups represented by G2 in —N(G2)(G2) are the same as or different from each other.

Plural groups represented by G3 in —N(G3)(G3) are the same as or different from each other.

Plural groups represented by G6 in —N(G6)(G6) are the same as or different from each other.

### Halogen Atom

In the description herein, specific examples (set of specific examples G11) of the “halogen atom” include a fluorine atom, a chlorine atom, a bromine atom, and an iodine atom.

### Substituted or Unsubstituted Fluoroalkyl Group

In the description herein, the “substituted or unsubstituted fluoroalkyl group” means a group formed by substituting at least one hydrogen atom bonded to the carbon atom constituting the alkyl group in the “substituted or unsubstituted alkyl group” by a fluorine atom, and encompasses a group formed by substituting all the hydrogen atoms bonded to the carbon atoms constituting the alkyl group in the “substituted or unsubstituted alkyl group” by fluorine atoms (i.e., a perfluoroalkyl group). The number of carbon atoms of the “unsubstituted fluoroalkyl group” is 1 to 50, preferably 1 to 30, and more preferably 1 to 18, unless otherwise indicated in the description. The “substituted fluoroalkyl group” means a group formed by substituting one or more hydrogen atoms of the “fluoroalkyl group” by a substituent. In the description herein, the “substituted fluoroalkyl group” encompasses a group formed by substituting one or more hydrogen atoms bonded to the carbon atom of the alkyl chain in the “substituted fluoroalkyl group” by a substituent, and a group formed by substituting one or more hydrogen atoms of the substituent in the “substituted fluoroalkyl group” by a substituent. Specific examples of the “unsubstituted fluoroalkyl group” include examples of groups formed by substituting one or more hydrogen atoms in each of the “alkyl group” (set of specific examples G3) by a fluorine atom.

### Substituted or Unsubstituted Haloalkyl Group

In the description herein, the “substituted or unsubstituted haloalkyl group” means a group formed by substituting at least one hydrogen atom bonded to the carbon atom constituting the alkyl group in the “substituted or unsubstituted alkyl group” by a halogen atom, and encompasses a group formed by substituting all the hydrogen atoms bonded to the carbon atoms constituting the alkyl group in the “substituted or unsubstituted alkyl group” by halogen atoms. The number of carbon atoms of the “unsubstituted haloalkyl group” is 1 to 50, preferably 1 to 30, and more preferably 1 to 18, unless otherwise indicated in the description. The “substituted haloalkyl group” means a group formed by substituting one or more hydrogen atoms of the “haloalkyl group” by a substituent. In the description herein, the “substituted haloalkyl group” encompasses a group formed by substituting one or more hydrogen atoms bonded to the carbon atom of the alkyl chain in the “substituted haloalkyl group” by a substituent, and a group formed by substituting one or more hydrogen atoms of the substituent in the “substituted haloalkyl group” by a substituent. Specific examples of the “unsubstituted haloalkyl group” include examples of groups formed by substituting one or more hydrogen atoms in each of the “alkyl group” (set of specific examples G3) by a halogen atom. A haloalkyl group can be referred to as a halogenated alkyl group in some cases.

### Substituted or Unsubstituted Alkoxy Group

In the description herein, specific examples of the “substituted or unsubstituted alkoxy group” include a group represented by —O(G3), wherein G3 represents the “substituted or unsubstituted alkyl group” described in the set of specific examples G3. The number of carbon atoms of the “unsubstituted alkoxy group” is 1 to 50, preferably 1 to 30, and more preferably 1 to 18, unless otherwise indicated in the description.

### Substituted or Unsubstituted Alkylthio Group

In the description herein, specific examples of the “substituted or unsubstituted alkylthio group” include a group represented by —S(G3), wherein G3 represents the “substituted or unsubstituted alkyl group” described in the set of specific examples G3. The number of carbon atoms of the “unsubstituted alkylthio group” is 1 to 50, preferably 1 to 30, and more preferably 1 to 18, unless otherwise indicated in the description.

### Substituted or Unsubstituted Aryloxy Group

In the description herein, specific examples of the “substituted or unsubstituted aryloxy group” include a group represented by —O(G1), wherein G1 represents the “substituted or unsubstituted aryl group” described in the set of specific examples G1. The number of ring carbon atoms of the “unsubstituted aryloxy group” is 6 to 50, preferably 6 to 30, and more preferably 6 to 18, unless otherwise indicated in the description.

### Substituted or Unsubstituted Arylthio Group

In the description herein, specific examples of the “substituted or unsubstituted arylthio group” include a group represented by —S(G1), wherein G1 represents the “substituted or unsubstituted aryl group” described in the set of specific examples G1. The number of ring carbon atoms of the “unsubstituted arylthio group” is 6 to 50, preferably 6 to 30, and more preferably 6 to 18, unless otherwise indicated in the description.

### Substituted or Unsubstituted Trialkylsilyl Group

In the description herein, specific examples of the “trialkylsilyl group” include a group represented by —Si(G3)(G3)(G3), wherein G3 represents the “substituted or unsubstituted alkyl group” described in the set of specific examples G3. Plural groups represented by G3 in —Si(G3)(G3)(G3) are the same as or different from each other. The number of carbon atoms of each of alkyl groups of the “substituted or unsubstituted trialkylsilyl group” is 1 to 50, preferably 1 to 20, and more preferably 1 to 6, unless otherwise indicated in the description.

### Substituted or Unsubstituted Aralkyl Group

In the description herein, specific examples of the “substituted or unsubstituted aralkyl group” include a group represented by -(G3)-(G1), wherein G3 represents the “substituted or unsubstituted alkyl group” described in the set of specific examples G3, and G1 represents the “substituted or unsubstituted aryl group” described in the set of specific examples G1. Accordingly, the “aralkyl group” is a group formed by substituting a hydrogen atom of an “alkyl group” by an “aryl group” as a substituent, and is one aspect of the “substituted alkyl group”. The “unsubstituted aralkyl group” is an “unsubstituted alkyl group” that is substituted by an “unsubstituted aryl group”, and the number of carbon atoms of the “unsubstituted aralkyl group” is 7 to 50, preferably 7 to 30, and more preferably 7 to 18, unless otherwise indicated in the description.

Specific examples of the “substituted or unsubstituted aralkyl group” include a benzyl group, a 1-phenylethyl group, a 2-phenylethyl group, a 1-phenylisopropyl group, a 2-phenylisopropyl group, a phenyl-t-butyl group, an α-naphthylmethyl group, a 1-α-naphthylethyl group, a 2-α-naphthylethyl group, a 1-α-naphthylisopropyl group, a 2-α-naphthylisopropyl group, a β-naphthylmethyl group, a 1-β-naphthylethyl group, a 2-β-naphthylethyl group, a 1-β-naphthylisopropyl group, and a 2-β-naphthylisopropyl group.

In the description herein, the substituted or unsubstituted aryl group is preferably a phenyl group, a p-biphenyl group, a m-biphenyl group, an o-biphenyl group, a p-terphenyl-4-yl group, a p-terphenyl-3-yl group, a p-terphenyl-2-yl group, a m-terphenyl-4-yl group, a m-terphenyl-3-yl group, a m-terphenyl-2-yl group, an o-terphenyl-4-yl group, an o-terphenyl-3-yl group, an o-terphenyl-2-yl group, a 1-naphthyl group, a 2-naphthyl group, an anthryl group, a phenanthryl group, a pyrenyl group, a chrysenyl group, a triphenylenyl group, a fluorenyl group, a 9,9′-spirobifluorenyl group, a 9,9-dimethylfluorenyl group, a 9,9-diphenylfluorenyl group, and the like, unless otherwise indicated in the description.

In the description herein, the substituted or unsubstituted heterocyclic group is preferably a pyridyl group, a pyrimidinyl group, a triazinyl group, a quinolyl group, an isoquinolyl group, a quinazolinyl group, a benzimidazolyl group, a phenanthrolinyl group, a carbazolyl group (e.g., a 1-carbazolyl, group, a 2-carbazolyl, group, a 3-carbazolyl, group, a 4-carbazolyl, group, or a 9-carbazolyl, group), a benzocarbazolyl group, an azacarbazolyl group, a diazacarbazolyl group, a dibenzofuranyl group, a naphthobenzofuranly group, an azadibenzofuranyl group, a diazadibenzofiranyl group, a dibenzothiophenyl group, a naphthobenzothiophenyl group, an azadibenzothiophenyl group, a diazadibenzothiophenyl group, a (9-phenyl)carbazolyl group (e.g., a (9-phenyl)carbazol-1-yl group, a (9-phenyl)carbazol-2-yl group, a (9-phenyl)carbazol-3-yl group, or a (9-phenyl)carbazol-4-yl group), a (9-biphenylyl)carbazolyl group, a (9-phenyl)phenylcarbazolyl group, a diphenylcarbazol-9-yl group, a phenylcarbazol-9-yl group, a phenyltriazinyl group, a biphenylyltriazinyl group, a diphenyltriazinyl group, a phenyldibenzofuranyl group, a phenyldibenzothiophenyl group, and the like, unless otherwise indicated in the description.

In the description herein, the carbazolyl group is specifically any one of the following groups unless otherwise indicated in the description.

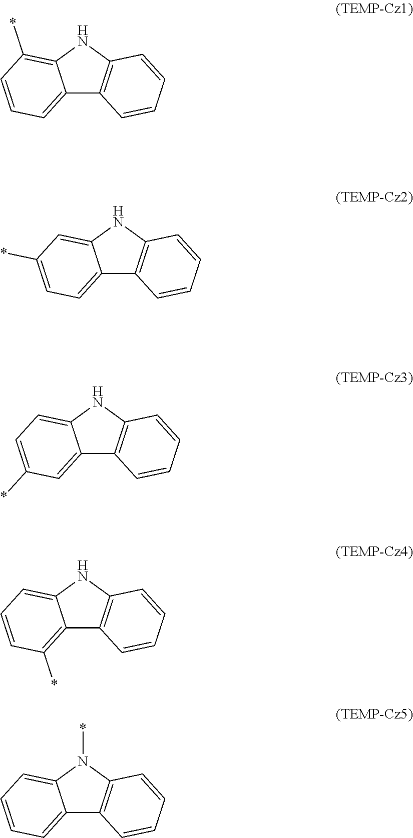 [SMILES: *c1cccc2c1[nH]c1ccccc12 ; *c1ccc2c(c1)[nH]c1ccccc12 ; *c1ccc2[nH]c3ccccc3c2c1 ; *c1cccc2[nH]c3ccccc3c12 ; *n1c2ccccc2c2ccccc21]

In the description herein, the (9-phenyl)carbazolyl group is specifically any one of the following groups unless otherwise indicated in the description.

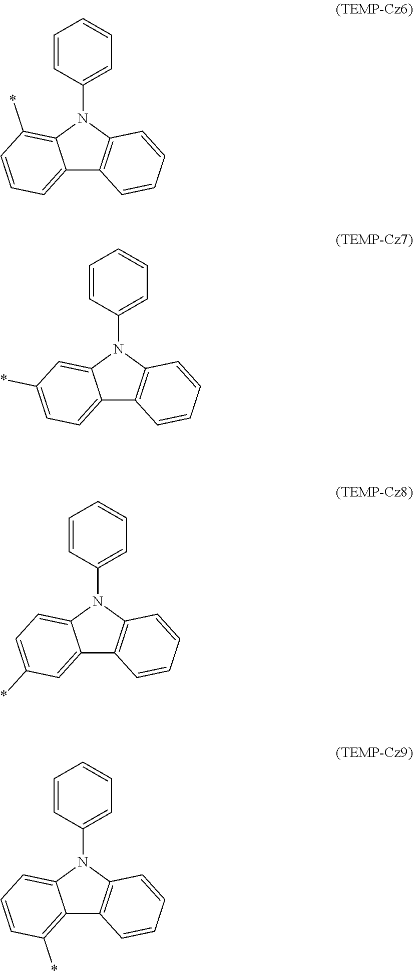 [SMILES: *c1cccc2c3ccccc3n(-c3ccccc3)c12 ; *c1ccc2c3ccccc3n(-c3ccccc3)c2c1 ; *c1ccc2c(c1)c1ccccc1n2-c1ccccc1 ; *c1cccc2c1c1ccccc1n2-c1ccccc1]

In the general formulae (TEMP-Czl) to (TEMP-Cz9), * represents a bonding site.

In the description herein, the dibenzofuranyl group and the dibenzothiophenyl group are specifically any one of the following groups unless otherwise indicated in the description.

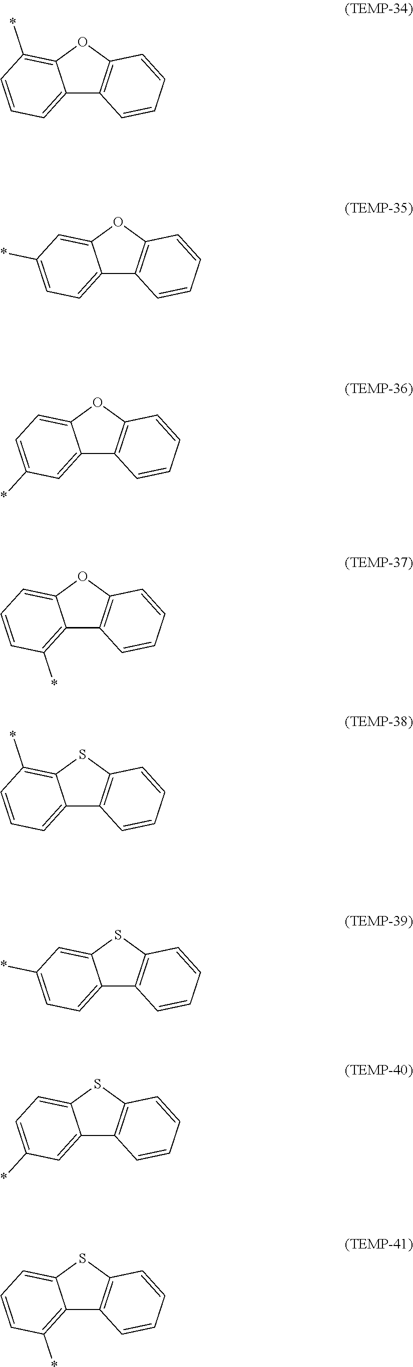

In the general formulae (TEMP-34) to (TEMP-41), * represents a bonding site.

In the description herein, the substituted or unsubstituted alkyl group is preferably a methyl group, an ethyl group, a propyl group, an isopropyl group, a n-butyl group, an isobutyl group, a t-butyl group, or the like unless otherwise indicated in the description.

### Substituted or Unsubstituted Arylene Group

In the description herein, the “substituted or unsubstituted arylene group” is a divalent group derived by removing one hydrogen atom on the aryl ring from the “substituted or unsubstituted aryl group” described above unless otherwise indicated in the description. Specific examples (set of specific examples G12) of the “substituted or unsubstituted arylene group” include divalent groups derived by removing one hydrogen atom on the aryl ring from the “substituted or unsubstituted aryl groups” described in the set of specific examples G1.

### Substituted or Unsubstituted Divalent Heterocyclic Group

In the description herein, the “substituted or unsubstituted divalent heterocyclic group” is a divalent group derived by removing one hydrogen atom on the heterocyclic ring from the “substituted or unsubstituted heterocyclic group” described above unless otherwise indicated in the description. Specific examples (set of specific examples G13) of the “substituted or unsubstituted divalent heterocyclic group” include divalent groups derived by removing one hydrogen atom on the heterocyclic ring from the “substituted or unsubstituted heterocyclic groups” described in the set of specific examples G2.

### Substituted or Unsubstituted Alkylene Group

In the description herein, the “substituted or unsubstituted alkylene group” is a divalent group derived by removing one hydrogen atom on the alkyl chain from the “substituted or unsubstituted alkyl group” described above unless otherwise indicated in the description. Specific examples (set of specific examples G14) of the “substituted or unsubstituted alkylene group” include divalent groups derived by removing one hydrogen atom on the alkyl chain from the “substituted or unsubstituted alkyl groups” described in the set of specific examples G3.

In the description herein, the substituted or unsubstituted arylene group is preferably any one of the groups represented by the following general formulae (TEMP-42) to (TEMP-68) unless otherwise indicated in the description.

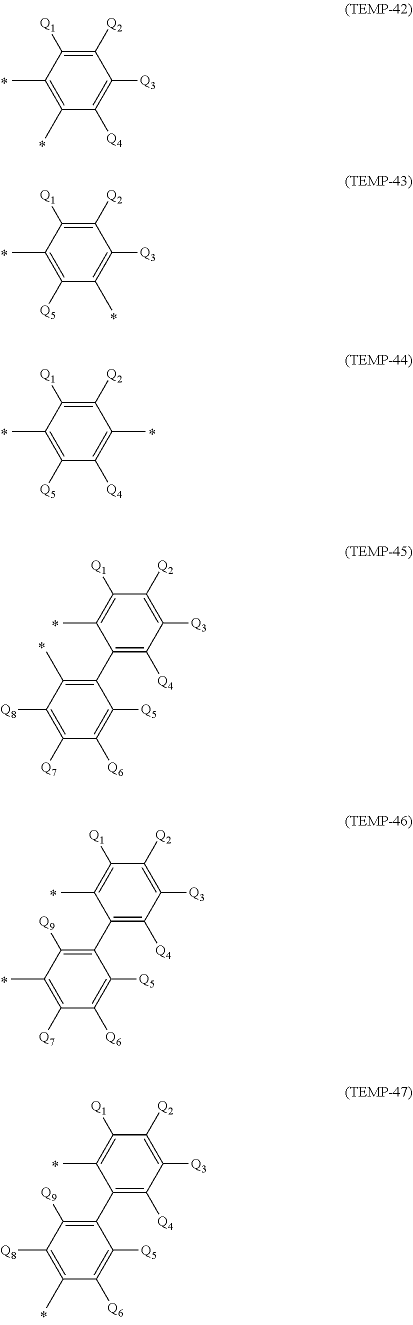

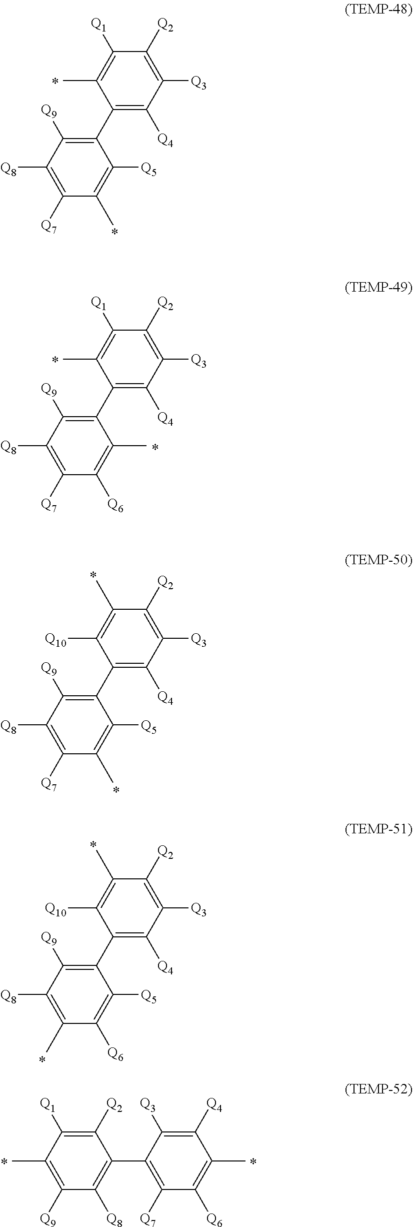

In the general formulae (TEMP-42) to (TEMP-52), Q₁ to Q₁₀ each independently represent a hydrogen atom or a substituent.

In the general formulae (TEMP-42) to (TEMP-52), * represents a bonding site.

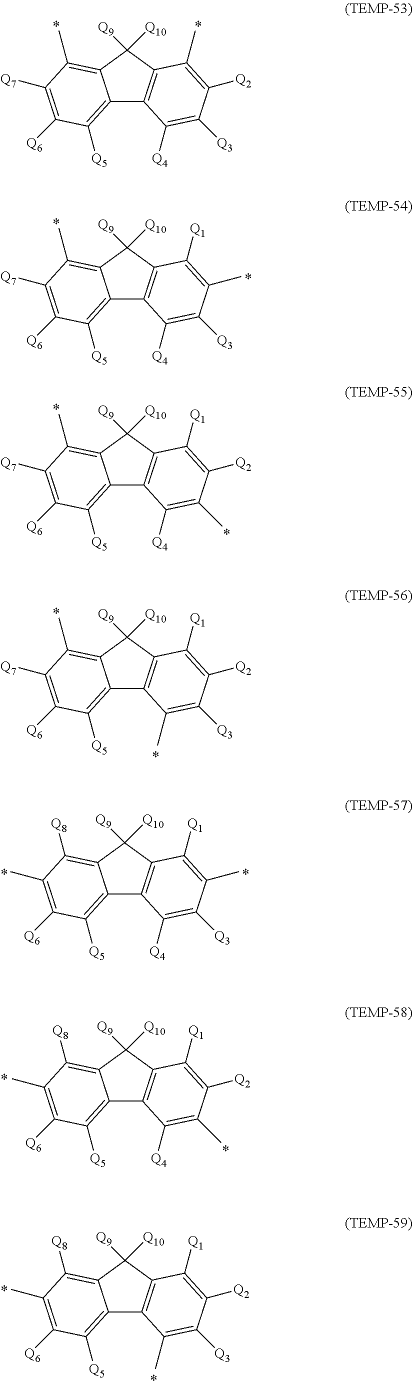

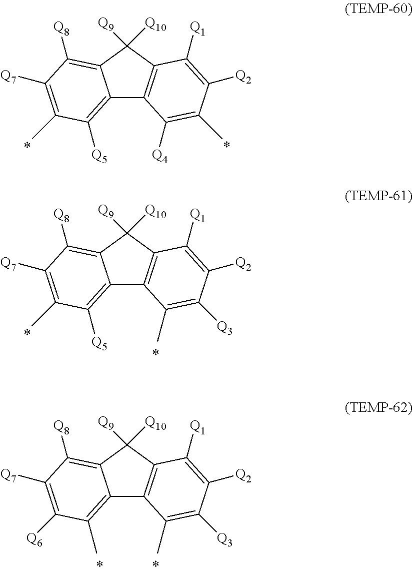

In the general formulae (TEMP-53) to (TEMP-62), Q₁ to Q₁₀ each independently represent a hydrogen atom or a substituent.

The formulae Q₉ and Q₁₀ can be bonded to each other to form a ring via a single bond.

In the general formulae (TEMP-53) to (TEMP-62), * represents a bonding site.

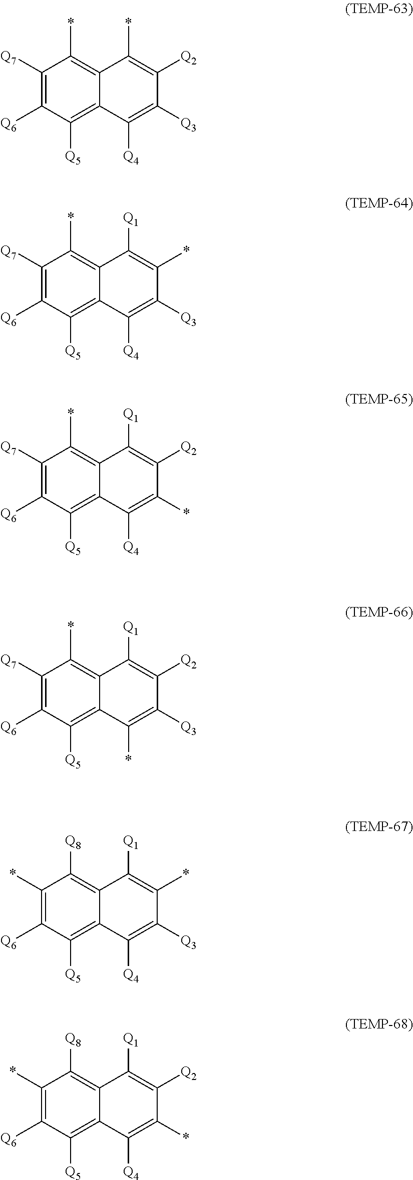

In the general formulae (TEMP-63) to (TEMP-68), Q₁ to Q₈ each independently represent a hydrogen atom or a substituent.

In the general formulae (TEMP-63) to (TEMP-68), * represents a bonding site.

In the description herein, the substituted or unsubstituted divalent heterocyclic group is preferably the groups represented by the following general formulae (TEMP-69) to (TEMP-102) unless otherwise indicated in the description.

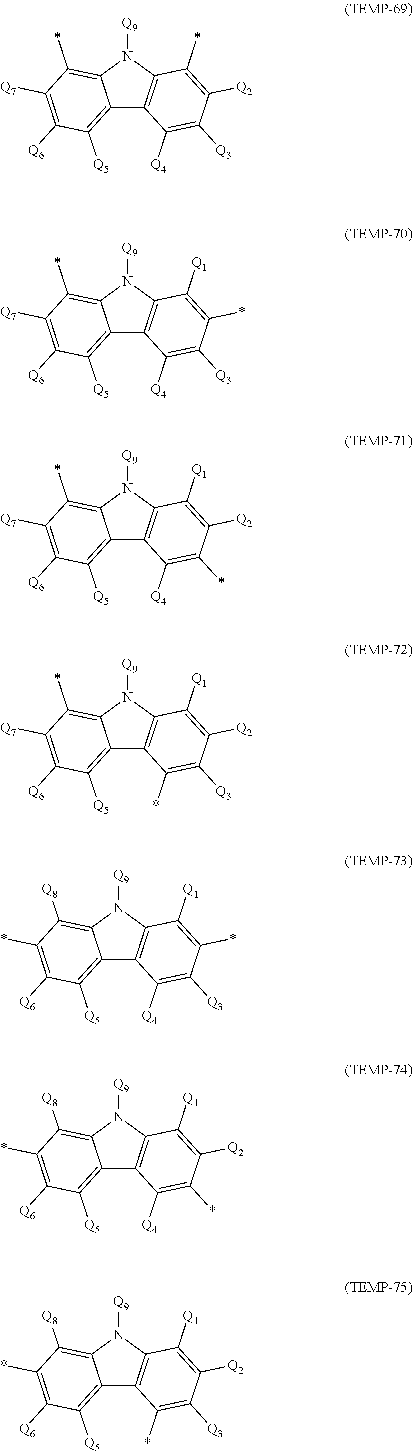

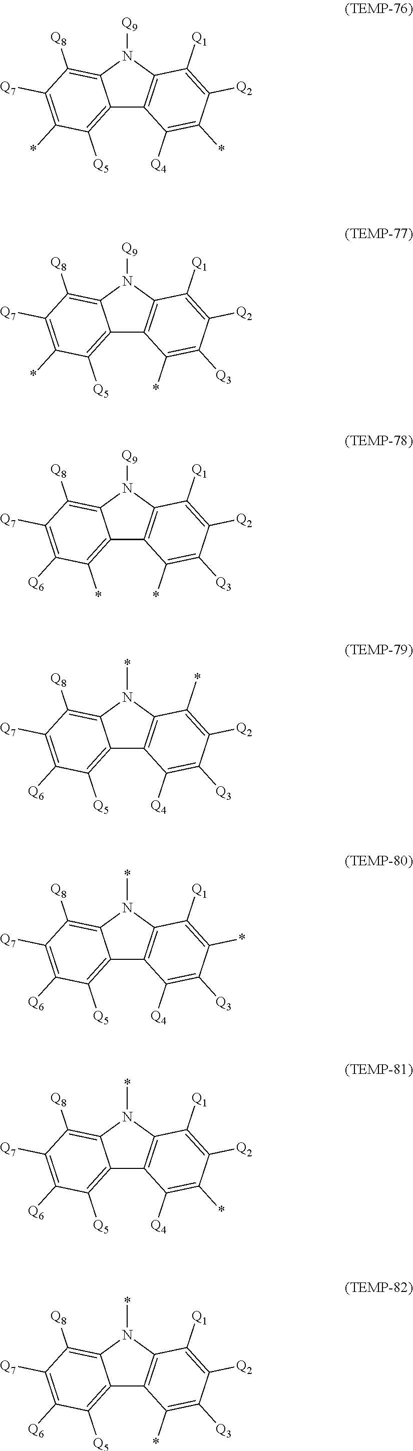

In the general formulae (TEMP-69) to (TEMP-82), Q₁ to Q₉ each independently represent a hydrogen atom or a substituent.

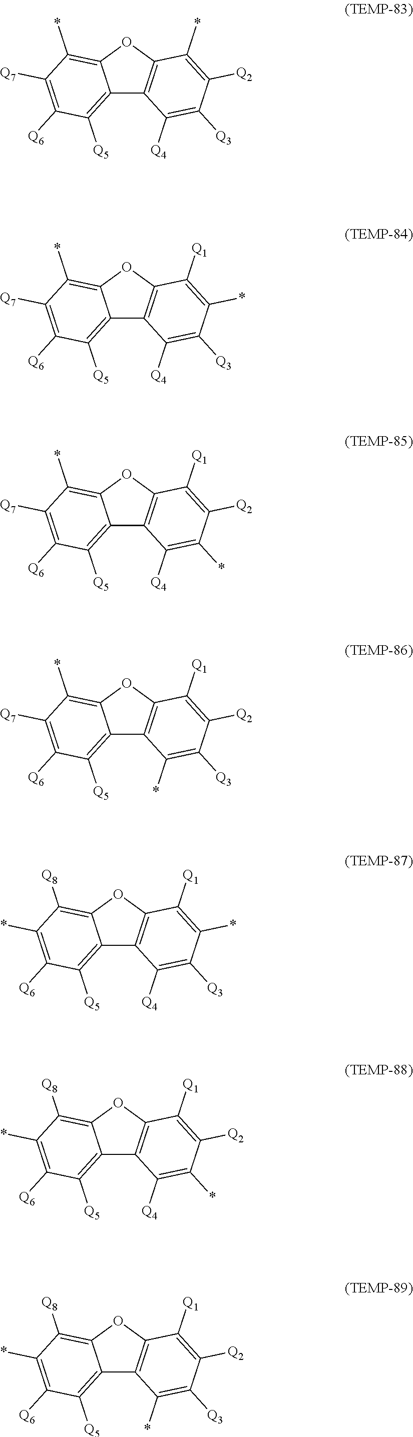

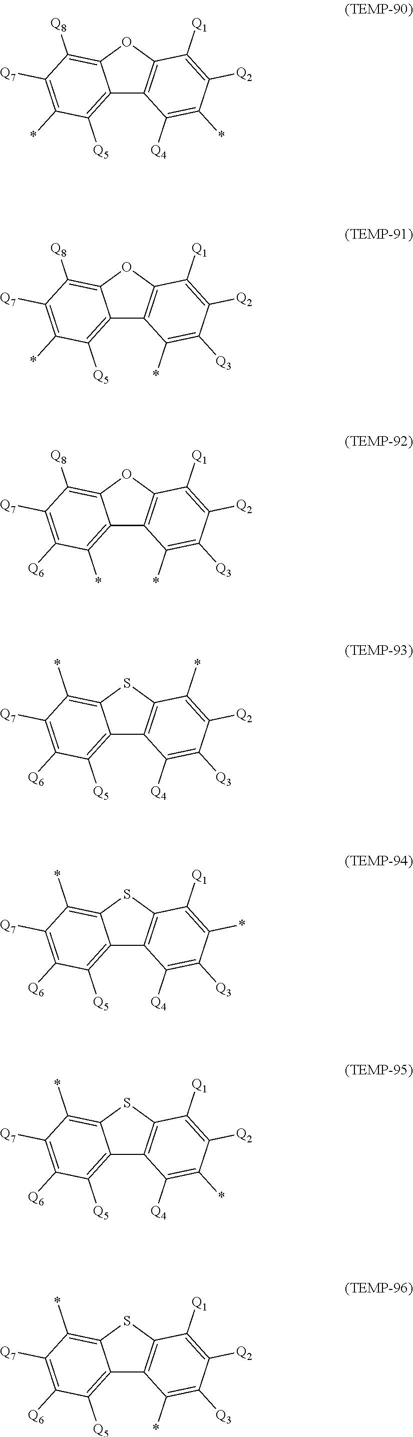

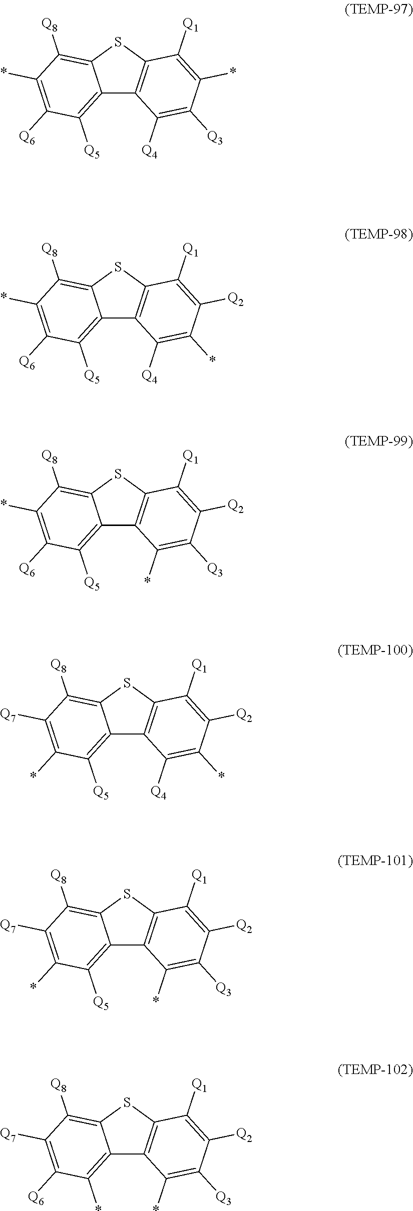

In the general formulae (TEMP-83) to (TEMP-102), Q₁ to Q₈ each independently represent a hydrogen atom or a substituent.

The above are the explanation of the “substituents in the description herein”.

### Case of Forming Ring by Bonding

In the description herein, the case where “one or more combinations of combinations each including adjacent two or more each are bonded to each other to form a substituted or unsubstituted monocyclic ring, or each are bonded to each other to form a substituted or unsubstituted condensed ring, or each are not bonded to each other” means a case where “one or more combinations of combinations each including adjacent two or more each are bonded to each other to form a substituted or unsubstituted monocyclic ring”, a case where “one or more combinations of combinations each including adjacent two or more each are bonded to each other to form a substituted or unsubstituted condensed ring”, and a case where “one or more combinations of combinations each including adjacent two or more each are not bonded to each other”.

In the description herein, the case where “one or more combinations of combinations each including adjacent two or more each are bonded to each other to form a substituted or unsubstituted monocyclic ring” and the case where “one or more combinations of combinations each including adjacent two or more each are bonded to each other to form a substituted or unsubstituted condensed ring” (which can be hereinafter collectively referred to as a “case forming a ring by bonding”) will be explained below. The cases will be explained for the anthracene compound represented by the following general formula (TEMP-103) having an anthracene core skeleton as an example.

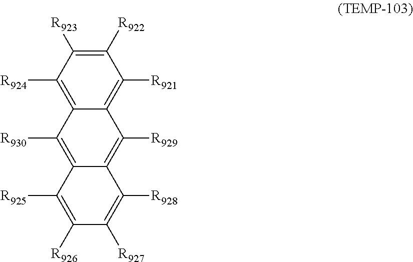

For example, in the case where “one or more combinations of combinations each including adjacent two or more each are bonded to each other to form a ring” among R₉₂₁ to R₉₃₀, the combinations each including adjacent two as one combination include a combination of R₉₂₁ and R₉₂₂, a combination of R₉₂ and R₉₂₃, a combination of R₉₂₃ and R₉₂₄, a combination of R₉₂₄ and R₉₃₀, a combination of R₉₃₀ and R₉₂₅, a combination of R₉₂₅ and R₉₂₆, a combination of R₉₂₆ and R₉₂₇, a combination of R₉₂₇ and R₉₂₈, a combination of R₉₂₈ and R₉₂₉, and a combination of R₉₂₉ and R₉₂₁.

The “one or more combinations” mean that two or more combinations each including adjacent two or more may form rings simultaneously. For example, in the case where R₉₂₁ and R₉₂ are bonded to each other to form a ring Q_{A}, and simultaneously R₉₂₅ and R₉₂₆ are bonded to each other to form a ring Q_{B}, the anthracene compound represented by the general formula (TEMP-103) is represented by the following general formula (TEMP-104).

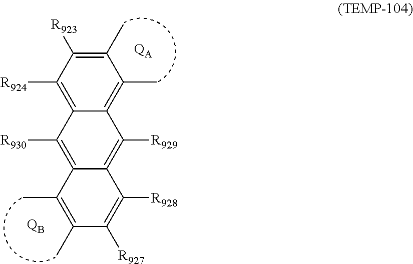

The case where the “combination including adjacent two or more forms rings” encompasses not only the case where adjacent two included in the combination are bonded as in the aforementioned example, but also the case where adjacent three or more included in the combination are bonded. For example, this case means that R₉₂₁ and R₉₂₂ are bonded to each other to form a ring Q_{A}, R₉₂ and R₉₂₃ are bonded to each other to form a ring Q_{C}, and adjacent three (R₉₂₁, R₉₂, and R₉₂₃) included in the combination are bonded to each other to form rings, which are condensed to the anthracene core skeleton, and in this case, the anthracene compound represented by the general formula (TEMP-103) is represented by the following general formula (TEMP-105). In the following general formula (TEMP-105), the ring Q_{A} and the ring Q_{C} share R₉₂₂.

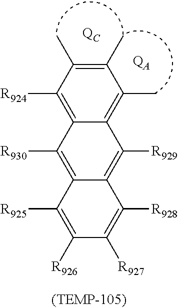

The formed “monocyclic ring” or “condensed ring” can be a saturated ring or an unsaturated ring in terms of structure of the formed ring itself. In the case where the “one combination including adjacent two” forms a “monocyclic ring” or a “condensed ring”, the “monocyclic ring” or the “condensed ring” may form a saturated ring or an unsaturated ring. For example, the ring Q_{A} and the ring Q_{B} formed in the general formula (TEMP-104) each are a “monocyclic ring” or a “condensed ring”. The ring Q_{A} and the ring Q_{C} formed in the general formula (TEMP-105) each are a “condensed ring”. The ring Q_{A} and the ring Q_{C} in the general formula (TEMP-105) form a condensed ring through condensation of the ring Q_{A} and the ring Q_{C}. In the case where the ring Q_{A} in the general formula (TMEP-104) is a benzene ring, the ring Q_{A} is a monocyclic ring. In the case where the ring Q_{A} in the general formula (TMEP-104) is a naphthalene ring, the ring Q_{A} is a condensed ring.

The “unsaturated ring” means an aromatic hydrocarbon ring or an aromatic heterocyclic ring. The “saturated ring” means an aliphatic hydrocarbon ring or a non-aromatic heterocyclic ring.

Specific examples of the aromatic hydrocarbon ring include the structures formed by terminating the groups exemplified as the specific examples in the set of specific examples G1 with a hydrogen atom.

Specific examples of the aromatic heterocyclic ring include the structures formed by terminating the aromatic heterocyclic groups exemplified as the specific examples in the set of specific examples G2 with a hydrogen atom.

Specific examples of the aliphatic hydrocarbon ring include the structures formed by terminating the groups exemplified as the specific examples in the set of specific examples G6 with a hydrogen atom.

The expression “to form a ring” means that the ring is formed only with the plural atoms of the core structure or with the plural atoms of the core structure and one or more arbitrary element. For example, the ring Q_{A} formed by bonding R₉₂₁ and R₉₂₂ to each other shown in the general formula (TEMP-104) means a ring formed with the carbon atom of the anthracene skeleton bonded to R₉₂₁, the carbon atom of the anthracene skeleton bonded to R₉₂₂, and one or more arbitrary element. As a specific example, in the case where the ring Q_{A} is formed with R₉₂₁ and R₉₂₂, and in the case where a monocyclic unsaturated ring is formed with the carbon atom of the anthracene skeleton bonded to R₉₂₁, the carbon atom of the anthracene skeleton bonded to R₉₂₂, and four carbon atoms, the ring formed with R₉₂₁ and R₉₂₂ is a benzene ring.

Herein, the “arbitrary element” is preferably at least one kind of an element selected from the group consisting of a carbon element, a nitrogen element, an oxygen element, and a sulfur element, unless otherwise indicated in the description. For the arbitrary element (for example, for a carbon element or a nitrogen element), a bond that does not form a ring can be terminated with a hydrogen atom or the like, and can be substituted by an “arbitrary substituent” described later. In the case where an arbitrary element other than a carbon element is contained, the formed ring is a heterocyclic ring.

The number of the “one or more arbitrary element” constituting the monocyclic ring or the condensed ring is preferably 2 or more and 15 or less, more preferably 3 or more and 12 or less, and further preferably 3 or more and 5 or less, unless otherwise indicated in the description.

What is preferred between the “monocyclic ring” and the “condensed ring” is the “monocyclic ring” unless otherwise indicated in the description.

What is preferred between the “saturated ring” and the “unsaturated ring” is the “unsaturated ring” unless otherwise indicated in the description.

The “monocyclic ring” is preferably a benzene ring unless otherwise indicated in the description.

The “unsaturated ring” is preferably a benzene ring unless otherwise indicated in the description.

In the case where the “one or more combinations of combinations each including adjacent two or more” each are “bonded to each other to form a substituted or unsubstituted monocyclic ring”, or each are “bonded to each other to form a substituted or unsubstituted condensed ring”, it is preferred that the one or more combinations of combinations each including adjacent two or more each are bonded to each other to form a substituted or unsubstituted “unsaturated ring” containing the plural atoms of the core skeleton and 1 or more and 15 or less at least one kind of elements selected from the group consisting of a carbon element, a nitrogen element, an oxygen element, and a sulfur element, unless otherwise indicated in the description.

In the case where the “monocyclic ring” or the “condensed ring” has a substituent, the substituent is, for example, an “arbitrary substituent” described later. In the case where the “monocyclic ring” or the “condensed ring” has a substituent, specific examples of the substituent include the substituents explained in the section “Substituents in Description” described above.

In the case where the “saturated ring” or the “unsaturated ring” has a substituent, the substituent is, for example, an “arbitrary substituent” described later. In the case where the “monocyclic ring” or the “condensed ring” has a substituent, specific examples of the substituent include the substituents explained in the section “Substituents in Description” described above.

The above are the explanation of the case where “one or more combinations of combinations each including adjacent two or more” each are “bonded to each other to form a substituted or unsubstituted monocyclic ring”, and the case where “one or more combinations of combinations each including adjacent two or more” each are “bonded to each other to form a substituted or unsubstituted condensed ring” (i.e., the “case forming a ring by bonding”).

### Substituent for “Substituted or Unsubstituted”

In one embodiment in the description herein, the substituent for the case of “substituted or unsubstituted” (which can be hereinafter referred to as an “arbitrary substituent”) is, for example, a group selected from the group consisting of
an unsubstituted alkyl group having 1 to 50 carbon atoms,
an unsubstituted alkenyl group having 2 to 50 carbon atoms,
an unsubstituted alkynyl group having 2 to 50 carbon atoms,
an unsubstituted cycloalkyl group having 3 to 50 ring carbon atoms,
Si(R₉₀₁)(R₉₀₂)(R₉₀₃),
O—(R₉₀₄),
S—(R₉₀₅),
N(R⁹⁰⁶)(R₉₀₇),
a halogen atom, a cyano group, a nitro group,
an unsubstituted aryl group having 6 to 50 ring carbon atoms, and
an unsubstituted heterocyclic group having 5 to 50 ring atoms,
wherein R₉₀₁ to R₉₀₇ each independently represent
a hydrogen atom,
a substituted or unsubstituted alkyl group having 1 to 50 carbon atoms
a substituted or unsubstituted cycloalkyl group having 3 to 50 ring carbon atoms,
a substituted or unsubstituted aryl group having 6 to 50 ring carbon atoms, or
a substituted or unsubstituted heterocyclic group having 5 to 50 ring atoms.

In the case where two or more groups each represented by R₉₀₁ exist, the two or more groups each represented by R₉₀₁ are the same as or different from each other,
in the case where two or more groups each represented by R₉₀₂ exist, the two or more groups each represented by R⁹⁰² are the same as or different from each other,
in the case where two or more groups each represented by R₉₀₃ exist, the two or more groups each represented by R₉₀₃ are the same as or different from each other,
in the case where two or more groups each represented by R₉₀₄ exist, the two or more groups each represented by R₉₀₄ are the same as or different from each other,
in the case where two or more groups each represented by R₉₀₅ exist, the two or more groups each represented by R₉₀₅ are the same as or different from each other,
in the case where two or more groups each represented by R₉₀₆ exist, the two or more groups each represented by R₉₀₆ are the same as or different from each other, and
in the case where two or more groups each represented by R₉₀₇ exist, the two or more groups each represented by R₉₀₇ are the same as or different from each other.

In one embodiment, the substituent for the case of “substituted or unsubstituted” can be a group selected from the group consisting of
an alkyl group having 1 to 50 carbon atoms,
an aryl group having 6 to 50 ring carbon atoms, and
a heterocyclic group having 5 to 50 ring atoms.

In one embodiment, the substituent for the case of “substituted or unsubstituted” can be a group selected from the group consisting of
an alkyl group having 1 to 18 carbon atoms,
an aryl group having 6 to 18 ring carbon atoms, and
a heterocyclic group having 5 to 18 ring atoms.

The specific examples of the groups for the arbitrary substituent described above are the specific examples of the substituent described in the section “Substituents in Description” described above.

In the description herein, the arbitrary adjacent substituents may form a “saturated ring” or an “unsaturated ring”, preferably form a substituted or unsubstituted saturated 5-membered ring, a substituted or unsubstituted saturated 6-membered ring, a substituted or unsubstituted unsaturated 5-membered ring, or a substituted or unsubstituted unsaturated 6-membered ring, and more preferably form a benzene ring, unless otherwise indicated.

In the description herein, the arbitrary substituent may further have a substituent unless otherwise indicated in the description. The definition of the substituent that the arbitrary substituent further has can be the same as the arbitrary substituent.

In the description herein, a numerical range shown by “AA to BB” means a range including the numerical value AA as the former of “AA to BB” as the lower limit value and the numerical value BB as the latter of “AA to BB” as the upper limit value.

The compound of the present invention will be described below.

The compound according to one aspect of the present invention is represented by the following formula (1).

In the following, the compound of the present invention, represented by each formula of the formula (1) and the formulae (1-1a) to (1-1f); formula (1-1a-1), formula (1-1b-1), formula (1-1c-1), formula (1-1d-1), formula (1-1a-2), formula (1-1b-2), formula (1-1c-2), and formula (1-1f-1); formulae (1-1a-3) to (1-1a-5), formulae (1-1b-3) to (1-1b-5), formulae (1-1c-3) to (1-1c-5), formulae (1-1d-3) to (1-1d-5), and formulae (1-1e-1) to (1-1e-3); formulae (1-1e-4) to (1-1e-6), and formulae (1-1f-3) to (1-1f-5) included in the formula (1), can be referred to simply as “compound (1)”.

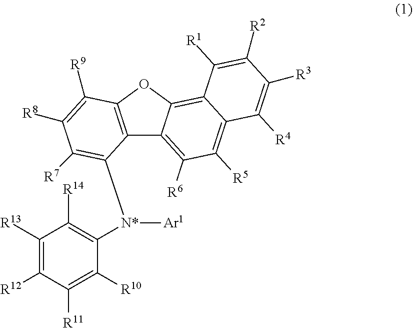

Symbols in the formula (1) and in the formulae included in the formula (1) are described below. The same symbols have the same meanings.

In the formula (1):
N* represents a central nitrogen atom.
R¹ to R⁹ each independently represent a hydrogen atom or a substituent A.

The substituent A is:
a halogen atom, a nitro group, a cyano group,
a substituted or unsubstituted alkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
a substituted or unsubstituted haloalkyl group having 1 to 50 carbon atoms,
a substituted or unsubstituted alkoxy group having 1 to 50 carbon atoms,
a substituted or unsubstituted haloalkoxy group having 1 to 50 carbon atoms,
a substituted or unsubstituted alkylthio group having 1 to 50 carbon atoms,
a substituted or unsubstituted aryloxy group having 6 to 50 ring carbon atoms,
a substituted or unsubstituted arylthio group having 6 to 50 ring carbon atoms,
a substituted or unsubstituted aralkyl group having 7 to 50 ring carbon atoms, or
a mono, di or tri-substituted silyl group having a substituent selected from a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, and a substituted or unsubstituted heterocyclic group having 5 to 50 ring atoms.

Details of the halogen atom are as described in the section of “substituents described in the present specification”.

Details of the substituted or unsubstituted alkyl group having 1 to 50 carbon atoms are as described in the section of “substituents described in the present specification”.

The unsubstituted alkyl group is preferably a methyl group, an ethyl group, an n-propyl group, an isopropyl group, an n-butyl group, an isobutyl group, an s-butyl group, or a t-butyl group, more preferably a methyl group, an ethyl group, an isopropyl group, or a t-butyl group, even more preferably a methyl group or a t-butyl group.

Details of the substituted or unsubstituted alkenyl group having 2 to 50 ring carbon atoms are as described in the section of “substituents described in the present specification”.

Details of the substituted or unsubstituted alkynyl group having 2 to 50 ring carbon atoms are as described in the section of “substituents described in the present specification”.

Details of the substituted or unsubstituted cycloalkyl group having 3 to 50 ring carbon atoms are as described in the section of “substituents described in the present specification”.

The unsubstituted cycloalkyl group is preferably a cyclopropyl group, a cyclobutyl group, a cyclopentyl group, a cyclohexyl group, a 1-adamantyl group, a 2-adamantyl group, a 1-norbornyl group, or a 2-norbornyl group, more preferably a cyclopropyl group, a cyclobutyl group, a cyclopentyl group or a cyclohexyl group, even more preferably a cyclopentyl group or a cyclohexyl group.

Details of the substituted or unsubstituted haloalkyl group having 1 to 50 carbon atoms are as described in the section of “substituents described in the present specification”, and preferred is a substituted or unsubstituted fluoroalkyl group having 1 to 50 carbon atoms.

The unsubstituted fluoroalkyl group is preferably a trifluoromethyl group, a 2,2,2-trifluoroethyl group, a pentafluoroethyl group, or a heptafluoropropyl group, more preferably a trifluoromethyl group.

Details of the substituted or unsubstituted alkoxy group having 1 to 50 carbon atoms are as described in the section of “substituents described in the present specification”.

The unsubstituted alkoxy group is preferably a methoxy group, an ethoxy group, a propoxy group or a t-butoxy group.

The substituted or unsubstituted haloalkoxy group having 1 to 50 carbon atoms is a group represented by —O(G15), where G15 is a substituted or unsubstituted haloalkyl group.

The substituted or unsubstituted haloalkoxy group having 1 to 50 carbon atoms is preferably a substituted or unsubstituted fluoroalkoxy group having 1 to 50 carbon atoms.

The unsubstituted fluoroalkoxy group is preferably a trifluoromethoxy group, a 2,2,2-trifluoroethoxy group, a pentafluoroethoxy group, or a heptafluoropropoxy group, more preferably a trifluoromethoxy group, a 2,2,2-trifluoroethoxy group, or a pentafluoroethoxy group, even more preferably a trifluoromethoxy group.

Details of the substituted or unsubstituted alkylthio group having 1 to 50 carbon atoms are as described in the section of “substituents described in the present specification”.

The unsubstituted alkylthio group is preferably a methylthio group, an ethylthio group, a propylthio group or a butylthio group.

Details of the substituted or unsubstituted aryloxy group having 6 to 50 ring carbon atoms are as described in the section of “substituents described in the present specification”.

The unsubstituted aryloxy group is preferably a phenoxy group, a biphenyloxy group, or a terphenyloxy group, more preferably a phenoxy group or a biphenyloxy group.

Details of the substituted or unsubstituted arylthio group having 6 to 50 ring carbon atoms are as described in the section of “substituents described in the present specification”.

The unsubstituted arylthio group is preferably a phenylthiol group or a tolylthio group.

Details of the substituted or unsubstituted aralkyl group having 7 to 50 ring carbon atoms are as described in the section of “substituents described in the present specification”.

The unsubstituted aralkyl group is preferably a benzyl group, a phenyl-t-butyl group, an α-naphthylmethyl group, a β-naphthylmethyl group, a 1-β-naphthylisopropyl group, or a 2-β-naphthylisopropyl group, more preferably a benzyl group, a phenyl-t-butyl group, an α-naphthylmethyl group or a β-naphthylmethyl group.

Details of the mono, di or tri-substituted silyl group are as described in the section of “substituents described in the present specification”.

The mono, di or tri-substituted silyl group is preferably a trimethylsilyl group, a triethylsilyl group, a t-butyldimethylsilyl group, a propyldimethylsilyl group, an isopropyldimethylsilyl group, a triphenylsilyl group, a phenyldimethylsilyl group, a t-butyldiphenylsilyl group, or a tritolylsilyl group, more preferably a trimethylsilyl group or a triphenylsilyl group.

One of R¹⁰ to R¹¹ is selected from a substituted or unsubstituted aryl group having 6 to 30 ring carbon atoms, and a substituted or unsubstituted heterocyclic group having 5 to 30 ring atoms. In the case where one of R¹⁰ to R¹⁴ is an aryl group, the ring constituting the aryl group is a 6-membered ring alone. R¹⁰ to R¹⁴ other than the one each are independently a hydrogen atom or the substituent A.

The substituted or unsubstituted aryl group having 6 to 30 ring carbon atoms that R¹⁰ to R¹⁴ represent are preferably each independently selected from the group consisting of a substituted or unsubstituted phenyl group, a substituted or unsubstituted biphenyl group, a substituted or unsubstituted terphenyl group, a substituted or unsubstituted naphthyl group, and a substituted or unsubstituted phenanthryl group.

In the substituted or unsubstituted terphenyl group, the terphenyl skeleton can have any structure of o-terphenyl, m-terphenyl and p-terphenyl.

The substituted or unsubstituted heteroaryl group having 5 to 30 ring atoms that R¹⁰ to R¹⁴ represent are preferably each independently selected from the group consisting of a substituted or unsubstituted dibenzofuranyl group, a substituted or unsubstituted dibenzothienyl group, a substituted or unsubstituted carbazolyl group, and a substituted or unsubstituted 9-carbazolyl group.

Ar¹ is represented by any of the following formulae (1-a) to (1-f):

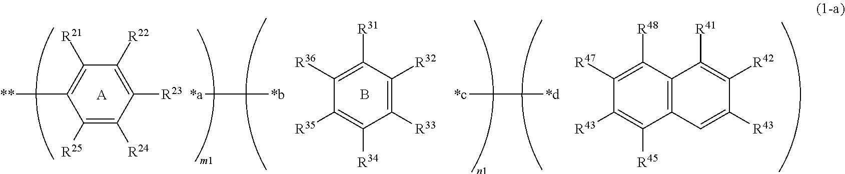

In the formula (1-a):
* indicates a bonding position to the central nitrogen atom N*;
R²¹ to R²⁵, and R³¹ to R³⁶ each independently represent a hydrogen atom or the substituent A;
R⁴¹ to R⁴⁸ each independently represent a hydrogen atom, a phenyl group or the substituent A:
m1 represents 0, 1 or 2;
n1 represents 0, 1 or 2;
m1+n1 is 0, 1 or 2, provided that:
when m1 and n1 are 0, one selected from R¹ to R⁴⁸ is a single bond bonding to the central nitrogen atom N*,
when m1 is 0 and n1 is 1 or 2, one selected from R³¹ to R³⁶ is a single bond bonding to the central nitrogen atom N*, and another one selected from R³¹ to R¹⁶ is a single bond bonding to *c, and one selected from R⁴¹ to R⁴⁸ is a single bond bonding to *d;
when n1 is 0 and m1 is 1 or 2, *a and *d bond together, one selected from R²¹ to R²⁵ is a single bond bonding to *a, and one selected from R⁴¹ to R⁴⁸ is a single bond bonding to *d;
when m1 and n1 are 1, one selected from R²¹ to R²⁵ is a single bond bonding to *a, one selected from R³¹ to R³⁶ is a single bond bonding to *b, another one selected from R³¹ to R³⁶ is a single bond bonding to *c, and one selected from R⁴¹ to R⁴⁸ is a single bond bonding to *d;
R²¹ to R²⁵ not being a single bond, R³¹ to R³⁶ not being a single bond, and R⁴¹ to R⁴⁸ not being a single bond do not bond to each other, and therefore do not form a cyclic structure.

In one aspect of the formula (1-a), m1 is 0 and n1 is 1, or m1 is 1 and n1 is 0; in another aspect, m1 is 0 and n1 is 2; in still another aspect, m1 is 1 and n1 is 1, and in another aspect, m1 is 2 and n1 is 0.

In the case where m1 is 2 and n1 is 0, the formula has two pairs of rings A's, and *b and *c bond. In one aspect, R²¹, R²², or R²³ on the first ring A is a single bond bonding to the bonding position ** on the second ring A, and R²¹, R²², or R²³ on the second ring A is a single bond bonding to *a.

In the case where m1 is 0 and n1 is 2, the formula has two pairs of rings B's, one selected from R³¹ to R³⁶ on the first ring B is a single bond bonding to the central nitrogen atom N*, another one selected from R³¹ to R³⁶ on the first ring B and one selected from R³¹ to R³⁶ on the second ring B bond to each other in a single bond, another one selected from R³¹ to R³⁶ on the second ring B is a single bond bonding to *c. In one aspect, R³¹ on the first ring B is a single bond bonding to the central nitrogen atom N*, R³¹ on the second ring B and R³², R³³, or R³⁴ on the first ring B bond to each other in a single bond, and R³², R³³, or R³⁴ on the second ring B is a single bond bonding to *c.

R²¹ to R²⁵ not being a single bond, and R³¹ to R²⁶ not being a single bond are preferably each independently a hydrogen atom, a halogen atom, a cyano group, a substituted or unsubstituted alkyl group having 1 to 50 carbon atoms, or a substituted or unsubstituted cycloalkyl group having 3 to 50 ring carbon atoms, more preferably a hydrogen atom, or a substituted or unsubstituted alkyl group having 1 to 50 carbon atoms.

Details of the halogen atom are as described in the section of “substituents described in the present specification”.

Details of the substituted or unsubstituted alkyl group having 1 to 50 carbon atoms are as described in the section of “substituents described in the present specification”.

The unsubstituted alkyl group is preferably a methyl group, an ethyl group, an n-propyl group, an isopropyl group, an n-butyl group, an isobutyl group, an s-butyl group, or a t-butyl group, more preferably a methyl group, an ethyl group, an isopropyl group, or a t-butyl group, even more preferably a methyl group or a t-butyl group.

Details of the substituted or unsubstituted cycloalkyl group having 3 to 50 ring carbon atoms are as described in the section of “substituents described in the present specification”.

The unsubstituted cycloalkyl group is preferably a cyclopropyl group, a cyclobutyl group, a cyclopentyl group, a cyclohexyl group, a 1-adamantyl group, a 2-adamantyl group, a 1-norbornyl group, or a 2-norbornyl group, more preferably a cyclopropyl group, a cyclobutyl group, a cyclopentyl group or a cyclohexyl group, even more preferably a cyclopentyl group or a cyclohexyl group.

R⁴¹ to R⁴⁸ not being a single bond are preferably each independently a hydrogen atom, a phenyl group, a halogen atom, a cyano group, a substituted or unsubstituted alkyl group having 1 to 50 carbon atoms, or a substituted or unsubstituted cycloalkyl group having 3 to 50 ring carbon atoms, more preferably a hydrogen atom, a phenyl group, or a substituted or unsubstituted alkyl group having 1 to 50 carbon atoms.

Details of the halogen atom are as described in the section of “substituents described in the present specification”.

Details of the substituted or unsubstituted alkyl group having 1 to 50 carbon atoms are as described in the section of “substituents described in the present specification”.

The unsubstituted alkyl group is preferably a methyl group, an ethyl group, an n-propyl group, an isopropyl group, an n-butyl group, an isobutyl group, an s-butyl group, or a t-butyl group, more preferably a methyl group, an ethyl group, an isopropyl group, or a t-butyl group, even more preferably a methyl group or a t-butyl group.

Details of the substituted or unsubstituted cycloalkyl group having 3 to 50 ring carbon atoms are as described in the section of “substituents described in the present specification”.

The unsubstituted cycloalkyl group is preferably a cyclopropyl group, a cyclobutyl group, a cyclopentyl group, a cyclohexyl group, a 1-adamantyl group, a 2-adamantyl group, a 1-norbornyl group, or a 2-norbornyl group, more preferably a cyclopropyl group, a cyclobutyl group, a cyclopentyl group or a cyclohexyl group, even more preferably a cyclopentyl group or a cyclohexyl group.

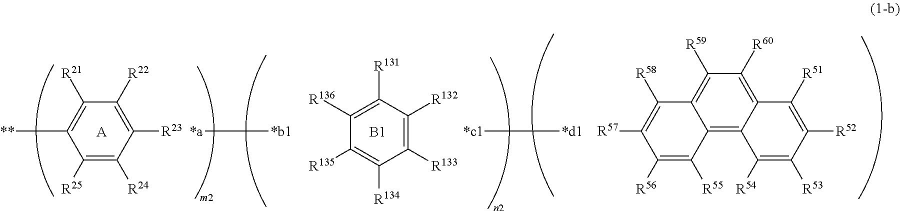

In the formula (1-b):
indicates a bonding position bonding to the central nitrogen atom N*;
R²¹ to R²⁵, and R¹³¹ to R¹³⁶ each independently represent a hydrogen atom or the substituent A;
R⁵¹ to R⁶⁰ each independently represent a hydrogen atom, a phenyl group or the substituent A;
m2 represents 0, 1 or 2;
n2 represents 0, 1 or 2;
m2+n2 is 0, 1 or 2, provided that:
when m2 and n2 are 0, one selected from R⁵¹ to R⁶⁰ is a single bond bonding to the central nitrogen atom N*;
when m2 is 0 and n2 is 1 or 2, one selected from R¹³¹ to R¹³⁶ is a single bond bonding to the central nitrogen atom N*, another one selected from R¹³¹ to R¹³⁶ is a single bond bonding to *c1, and one selected from R⁵¹ to R⁶⁰ is a single bond bonding to *d1;
when n2 is 0 and m2 is 1 or 2, *a and *d1 bond together, one selected from R²¹ to R²⁵ is a single bond bonding to *a, and one selected from R⁵¹ to R⁶⁰ is a single bond bonding *d1;
when m2 and n2 are 1, one selected from R²¹ to R²⁵ is a single bond bonding to *a, one selected from R¹³¹ to R¹³⁶ is a single bond bonding to *b1, another one selected from R¹³¹ to R¹³⁶ is a single bond bonding to *c1, and one selected from R⁵¹ to R⁶⁰ is a single bond bonding to *d1;
R²¹ to R²⁵ not being a single bond, R¹³¹ to R¹³⁶ not being a single bond, and R⁵¹ to R⁶⁰ not being a single bond do not bond to each other and therefore do not form a cyclic structure.

In one aspect of the formula (1-b), m2 is 0 and n2 is 1, or m2 is 1 and n2 is 0; in another aspect, m2 is 0 and n2 is 2; in still another aspect, m2 is 1 and n2 is 1, and in another aspect, m2 is 2 and n2 is 0.

In the case where m2 is 2 and n2 is 0, the formula has two pairs of rings A's, and *b1 and *c1 bond. In one aspect, R²¹, R²², or R²³ on the first ring A is a single bond bonding to the bonding position ** on the second ring A, and R²¹, R²², or R²³ on the second ring A is a single bond bonding to *a.

In the case where m2 is 0 and n2 is 2, the formula has two pairs of rings B1's, one selected from R¹³¹ to R¹³⁶ on the first ring B1 is a single bond bonding to the central nitrogen atom N*, another one selected from R¹³¹ to R¹³⁶ on the first ring B1 and one selected from R¹³¹ to R¹³⁶ on the second ring B1 bond to each other in a single bond, another one selected from R¹³¹ to R¹³⁶ on the second ring B1 is a single bond bonding to *c1. In one embodiment, R¹³¹ on the first ring B1 is a single bond bonding to the central nitrogen atom N*, R¹³¹ on the second ring B1 and R¹³², R¹³³, or R¹³⁴ on the first ring B1 bond to each other in a single bond, and R¹³², R¹³³, or R¹³⁴ on the second ring B1 is a single bond bonding to *c1.

R²¹ to R²⁵ not being a single bond, and R¹³¹ to R¹³⁶ not being a single bond are preferably each independently a hydrogen atom, a halogen atom, a cyano group, a substituted or unsubstituted alkyl group having 1 to 50 carbon atoms, or a substituted or unsubstituted cycloalkyl group having 3 to 50 ring carbon atoms, more preferably a hydrogen atom, or a substituted or unsubstituted alkyl group having 1 to 50 carbon atoms.

Details of the halogen atom are as described in the section of “substituents described in the present specification”.

Details of the substituted or unsubstituted alkyl group having 1 to 50 carbon atoms are as described in the section of “substituents described in the present specification”.

The unsubstituted alkyl group is preferably a methyl group, an ethyl group, an n-propyl group, an isopropyl group, an n-butyl group, an isobutyl group, an s-butyl group, or a t-butyl group, more preferably a methyl group, an ethyl group, an isopropyl group, or a t-butyl group, even more preferably a methyl group or a t-butyl group.

Details of the substituted or unsubstituted cycloalkyl group having 3 to 50 ring carbon atoms are as described in the section of “substituents described in the present specification”.

The unsubstituted cycloalkyl group is preferably a cyclopropyl group, a cyclobutyl group, a cyclopentyl group, a cyclohexyl group, a 1-adamantyl group, a 2-adamantyl group, a 1-norbornyl group, or a 2-norbornyl group, more preferably a cyclopropyl group, a cyclobutyl group, a cyclopentyl group or a cyclohexyl group, even more preferably a cyclopentyl group or a cyclohexyl group.

R⁵¹ to R⁶⁰ not being a single bond are preferably each independently a hydrogen atom, a phenyl group, a halogen atom, a cyano group, a substituted or unsubstituted alkyl group having 1 to 50 carbon atoms, or a substituted or unsubstituted cycloalkyl group having 3 to 50 ring carbon atoms, more preferably a hydrogen atom, a phenyl group, or a substituted or unsubstituted alkyl group having 1 to 50 carbon atoms.

Details of the halogen atom are as described in the section of “substituents described in the present specification”.

Details of the substituted or unsubstituted alkyl group having 1 to 50 carbon atoms are as described in the section of “substituents described in the present specification”.

The unsubstituted alkyl group is preferably a methyl group, an ethyl group, an n-propyl group, an isopropyl group, an n-butyl group, an isobutyl group, an s-butyl group, or a t-butyl group, more preferably a methyl group, an ethyl group, an isopropyl group, or a t-butyl group, even more preferably a methyl group or a t-butyl group.

Details of the substituted or unsubstituted cycloalkyl group having 3 to 50 ring carbon atoms are as described in the section of “substituents described in the present specification”.

The unsubstituted cycloalkyl group is preferably a cyclopropyl group, a cyclobutyl group, a cyclopentyl group, a cyclohexyl group, a 1-adamantyl group, a 2-adamantyl group, a 1-norbornyl group, or a 2-norbornyl group, more preferably a cyclopropyl group, a cyclobutyl group, a cyclopentyl group or a cyclohexyl group, even more preferably a cyclopentyl group or a cyclohexyl group.

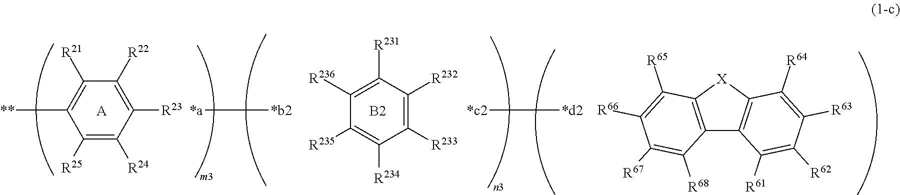

In the formula (1-c):
* indicates a bonding position bonding to the central nitrogen atom N*;
X represents an oxygen atom, a sulfur atom, or NR^{a};
R^{a} represents a hydrogen atom, a substituted or unsubstituted alkyl group having 1 to 50 carbon atoms, or a substituted or unsubstituted aryl group having 6 to 50 ring carbon atoms;
R²¹ to R²⁵, and R²³¹ to R²³⁶ each independently represent a hydrogen atom or the substituent A.
R⁶¹ to R⁶⁸ each independently represent a hydrogen atom, a phenyl group, or the substituent A.
m3 represents 0, 1 or 2;
n3 represents 0, 1 or 2;
m3+n3 is 0, 1 or 2, provided that:
when m3 and n3 are 0, one selected from R⁶¹ to R⁶⁸ is a single bond bonding to the central nitrogen atom N*,
when m3 is 0 and n3 is 1 or 2, one selected from R²³¹ to R²³⁶ is a single bond bonding to the central nitrogen atom N*, another one selected from R²³¹ to R²³⁶ is a single bond bonding to *c2, and one selected from R⁶¹ to R⁶⁸ is a single bond bonding to *d2;
when n3 is 0 and m3 is 1 or 2, *a and *d2 bond together, one selected from R²¹ to R²⁵ is a single bond bonding to *a, and one selected from R⁶¹ to R⁶⁸ is a single bond bonding to *d2;
when m3 and n3 are 1, one selected from R²¹ to R²⁵ is a single bond bonding to *a, one selected from R²³¹ to R²³⁶ is a single bond bonding to *b2, another one selected from R²³¹ to R²³⁶ is a single bond bonding to *c2, and one selected from R⁶¹ to R⁶⁸ is a single bond bonding to *d2;
R²¹ to R²⁵ not being a single bond, and R²³¹ to R²³⁶ not being a single bond do not bond to each other and therefore do not form a cyclic structure;
one or more adjacent pairs of R⁶¹ to R⁶⁸ not being a single bond may or may not bond to each other to form a substituted or unsubstituted benzene ring.

In one aspect of the formula (1-c), m3 is 0 and n3 is 1, or m3 is 1 and n3 is 0; in another aspect, m3 is 0 and n3 is 2; in still another aspect, m3 is 1 and n3 is 1, and in another aspect, m3 is 2 and n3 is 0.

In the case where m3 is 2 and n3 is 0, the formula has two pairs of rings A's, and *b2 and *c2 bond. In one aspect, R²¹, R²², or R²³ on the first ring A is a single bond bonding to the bonding position ** on the second ring A, and R²¹, R²², or R²³ on the second ring A is a single bond bonding to *a.

In the case where m3 is 0 and n3 is 2, the formula has two pairs of rings B2's, one selected from R²³¹ to R²³⁶ on the first ring B2 is a single bond bonding to the central nitrogen atom N*, another one selected from R²³¹ to R²³⁶ on the first ring B2 and one selected from R²³¹ to R²³⁶ on the second ring B2 bond to each other in a single bond, another one selected from R²³¹ to R²³⁶ on the second ring B2 is a single bond bonding to *c2. In one aspect, R²³¹ on the first ring B2 is a single bond bonding to the central nitrogen atom N*, R²³¹ on the second ring B2 and R²³², R²³³, or R²³⁴ on the first ring B2 bond to each other in a single bond, and R²³², R²³³, or R²³⁴ on the second ring B2 is a single bond bonding to *c2.

R²¹ to R²⁵ not being a single bond, and R²³¹ to R²³⁶ not being a single bond are preferably each independently a hydrogen atom, a halogen atom, a cyano group, a substituted or unsubstituted alkyl group having 1 to 50 carbon atoms, or a substituted or unsubstituted cycloalkyl group having 3 to 50 ring carbon atoms, more preferably a hydrogen atom, or a substituted or unsubstituted alkyl group having 1 to 50 carbon atoms.

Details of the halogen atom are as described in the section of “substituents described in the present specification”.

Details of the substituted or unsubstituted alkyl group having 1 to 50 carbon atoms are as described in the section of “substituents described in the present specification”.

The unsubstituted alkyl group is preferably a methyl group, an ethyl group, an n-propyl group, an isopropyl group, an n-butyl group, an isobutyl group, an s-butyl group, or a t-butyl group, more preferably a methyl group, an ethyl group, an isopropyl group, or a t-butyl group, even more preferably a methyl group or a t-butyl group.

Details of the substituted or unsubstituted cycloalkyl group having 3 to 50 ring carbon atoms are as described in the section of “substituents described in the present specification”.

The unsubstituted cycloalkyl group is preferably a cyclopropyl group, a cyclobutyl group, a cyclopentyl group, a cyclohexyl group, a 1-adamantyl group, a 2-adamantyl group, a 1-norbornyl group, or a 2-norbornyl group, more preferably a cyclopropyl group, a cyclobutyl group, a cyclopentyl group or a cyclohexyl group, even more preferably a cyclopentyl group or a cyclohexyl group.

R⁶¹ to R⁶⁸ not a single bond are preferably each independently a hydrogen atom, a phenyl group, a halogen atom, a cyano group, a substituted or unsubstituted alkyl group having 1 to 50 carbon atoms, or a substituted or unsubstituted cycloalkyl group having 3 to 50 ring carbon atoms, more preferably a hydrogen atom, a phenyl group, or a substituted or unsubstituted alkyl group having 1 to 50 carbon atoms.

Details of the halogen atom are as described in the section of “substituents described in the present specification”.

Details of the substituted or unsubstituted alkyl group having 1 to 50 carbon atoms are as described in the section of “substituents described in the present specification”.

The unsubstituted alkyl group is preferably a methyl group, an ethyl group, an n-propyl group, an isopropyl group, an n-butyl group, an isobutyl group, an s-butyl group, or a t-butyl group, more preferably a methyl group, an ethyl group, an isopropyl group, or a t-butyl group, even more preferably a methyl group or a t-butyl group.

Details of the substituted or unsubstituted cycloalkyl group having 3 to 50 ring carbon atoms are as described in the section of “substituents described in the present specification”.

The unsubstituted cycloalkyl group is preferably a cyclopropyl group, a cyclobutyl group, a cyclopentyl group, a cyclohexyl group, a 1-adamantyl group, a 2-adamantyl group, a 1-norbornyl group, or a 2-norbornyl group, more preferably a cyclopropyl group, a cyclobutyl group, a cyclopentyl group or a cyclohexyl group, even more preferably a cyclopentyl group or a cyclohexyl group.

R^{a} is preferably a hydrogen atom, an unsubstituted alkyl group having 1 to 50 carbon atom, or an unsubstituted aryl group having 6 to 50 ring carbon atoms.

The unsubstituted alkyl group is preferably a methyl group, an ethyl group, an n-propyl group, an isopropyl group, an n-butyl group, an isobutyl group, an s-butyl group, or a t-butyl group, more preferably a methyl group, an ethyl group, an isopropyl group, or a t-butyl group.

The unsubstituted aryl group is preferably a phenyl group, a p-biphenyl group, an m-biphenyl group, an o-biphenyl group, a 1-naphthyl group, a 2-naphthyl group, an anthryl group, a benzanthryl group, a phenanthryl group or a triphenylene group.

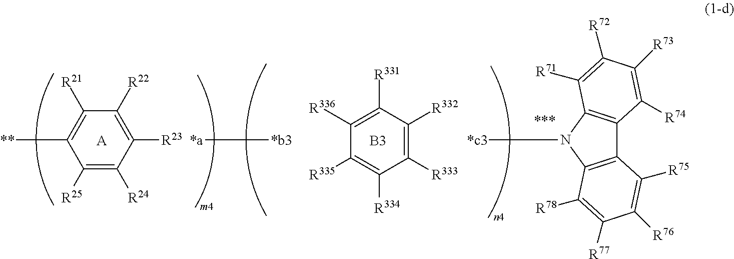

In the formula (1-d):
* indicates a bonding position bonding to the central nitrogen atom N*.
R²¹ to R²⁵, and R³³¹ to R³³⁶ each independently represent a hydrogen atom or the substituent A.
R⁷¹ to R⁷⁸ each independently represent a hydrogen atom, a phenyl group or the substituent A.
m4 represents 0, 1 or 2;
n4 represents 0, 1 or 2;
m4+n4 is 1 or 2, provided that:
when m4 is 0 and n4 is 1 or 2, one selected from R³³¹ to R³³⁶ is a single bond bonding the central nitrogen atom N*, and another one selected from R³³¹ to R³³⁶ is a single bond bonding to *c3;
when n4 is 0 and m4 is 1 or 2, one selected from R²¹ to R²⁵ is a single bond bonding to the nitrogen atom N***;
when m4 and n4 are 1, one selected from R²¹ to R²⁵ is a single bond bonding to *a, one selected from R³³¹ to R³³⁶ is a single bond to *b3, and another one selected from R³³¹ to R³³⁶ is a single bond bonding to *c3.
R²¹ to R²⁵ not being a single bond, and R³³¹ to R³³⁵ and R⁷¹ to R⁷⁸ not being a single bond do not bond to each other and therefore do not form a cyclic structure.

In one aspect of the formula (1-d), m4 is 0 and n4 is 1, or m4 is 1 and n4 is 0; in another aspect, m4 is 0 and n4 is 2; in still another aspect, m4 is 2 and n4 is 0.

In the case where m4 is 2 and n4 is 0, the formula has two pairs of rings A's, and *b2 and *c2 bond. In one aspect, R²¹, R²², or R²³ on the first ring A is a single bond bonding to the bonding position ** on the second ring A, and R²¹, R²², or R²³ on the second ring A is a single bond bonding to *a.

In the case where m4 is 0 and n4 is 2, the formula has two pairs of rings B3's, one selected from R³³¹ to R³³⁶ on the first ring B3 is a single bond bonding to the central nitrogen atom N*, another one selected from R³³¹ to R³³⁶ on the first ring B3 and one selected from R³³¹ to R³³⁶ on the second ring B3 bond to each other in a single bond, and another one selected from R³³¹ to R³³⁶ on the second ring B3 is a single bond bonding to *c3. In one aspect, R³³¹ on the first ring B3 is a single bond bonding to the central nitrogen atom N*, R³³¹ on the second ring B3 and R³³², R³³³ or R³³⁴ on the first ring B3 bond to each other in a single bond, and R³³², R³³³, or R³³⁴ on the second ring B3 is a single bond bonding to *c3.

R²¹ to R²⁵ not being a single bond, and R³³¹ to R³³⁶ not being a single bond are preferably each independently a hydrogen atom, a halogen atom, a cyano group, a substituted or unsubstituted alkyl group having 1 to 50 carbon atoms, or a substituted or unsubstituted cycloalkyl group having 3 to 50 ring carbon atoms, more preferably a hydrogen atom, or a substituted or unsubstituted alkyl group having 1 to 50 carbon atoms.

Details of the halogen atom are as described in the section of “substituents described in the present specification”.

Details of the substituted or unsubstituted alkyl group having 1 to 50 carbon atoms are as described in the section of “substituents described in the present specification”.

The unsubstituted alkyl group is preferably a methyl group, an ethyl group, an n-propyl group, an isopropyl group, an n-butyl group, an isobutyl group, an s-butyl group, or a t-butyl group, more preferably a methyl group, an ethyl group, an isopropyl group, or a t-butyl group, even more preferably a methyl group or a t-butyl group.

Details of the substituted or unsubstituted cycloalkyl group having 3 to 50 ring carbon atoms are as described in the section of “substituents described in the present specification”.

The unsubstituted cycloalkyl group is preferably a cyclopropyl group, a cyclobutyl group, a cyclopentyl group, a cyclohexyl group, a 1-adamantyl group, a 2-adamantyl group, a 1-norbornyl group, or a 2-norbornyl group, more preferably a cyclopropyl group, a cyclobutyl group, a cyclopentyl group or a cyclohexyl group, even more preferably a cyclopentyl group or a cyclohexyl group.

R⁷¹ to R⁷⁸ are preferably each independently a hydrogen atom, a phenyl group, a halogen atom, a cyano group, a substituted or unsubstituted alkyl group having 1 to 50 carbon atoms, or a substituted or unsubstituted cycloalkyl group having 3 to 50 ring carbon atoms, more preferably a hydrogen atom, a phenyl group, or a substituted or unsubstituted alkyl group having 1 to 50 carbon atoms.

Details of the halogen atom are as described in the section of “substituents described in the present specification”.

Details of the substituted or unsubstituted alkyl group having 1 to 50 carbon atoms are as described in the section of “substituents described in the present specification”.

The unsubstituted alkyl group is preferably a methyl group, an ethyl group, an n-propyl group, an isopropyl group, an n-butyl group, an isobutyl group, an s-butyl group, or a t-butyl group, more preferably a methyl group, an ethyl group, an isopropyl group, or a t-butyl group, even more preferably a methyl group or a t-butyl group.

Details of the substituted or unsubstituted cycloalkyl group having 3 to 50 ring carbon atoms are as described in the section of “substituents described in the present specification”.

The unsubstituted cycloalkyl group is preferably a cyclopropyl group, a cyclobutyl group, a cyclopentyl group, a cyclohexyl group, a 1-adamantyl group, a 2-adamantyl group, a 1-norbornyl group, or a 2-norbornyl group, more preferably a cyclopropyl group, a cyclobutyl group, a cyclopentyl group or a cyclohexyl group, even more preferably a cyclopentyl group or a cyclohexyl group.

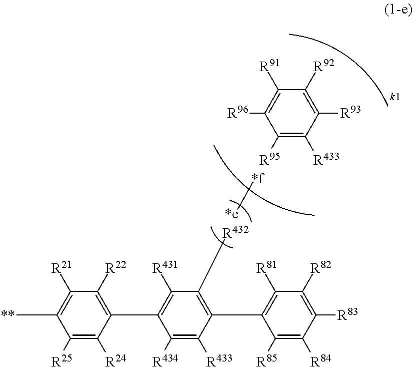

In the formula (1-e):
* indicates a bonding position bonding to the central nitrogen atom N*.
R²¹, R²², R²⁴, R²⁵, R⁴³¹ to R⁴³⁴, R⁸¹ to R⁸⁵, and R⁹¹ to R⁹⁶ each independently represent a hydrogen atom or the substituent A.
k1 represents 0 or 1, provided that:
when k1 is 1, R⁴³² is a single bond bonding to *e, and one selected from R⁹¹ to R⁹⁶ is a single bond bonding to *f.
R⁹¹ to R⁹⁶ not being a single bond, and R⁴³², R⁴³¹, R⁴³³, R⁴³⁴, R²¹, R²², R²⁴, R²⁵, and R⁸¹ to R⁸⁵ not being a single bond do not bond to each other and therefore do not form a cyclic structure.

R²¹ to R²⁵, R⁴³¹, R⁴³³, R⁴³⁴, R⁸¹ to R⁸⁵, R⁴³² that is not a single bond, and R⁹¹ tO R⁹⁶ that are not a single bond are preferably each independently a hydrogen atom, a halogen atom, a cyano group, a substituted or unsubstituted alkyl group having 1 to 50 carbon atoms, or a substituted or unsubstituted cycloalkyl group having 3 to 50 ring carbon atoms, more preferably a hydrogen atom, or a substituted or unsubstituted alkyl group having 1 to 50 carbon atoms.

Details of the halogen atom are as described in the section of “substituents described in the present specification”.

Details of the substituted or unsubstituted alkyl group having 1 to 50 carbon atoms are as described in the section of “substituents described in the present specification”.

The unsubstituted alkyl group is preferably a methyl group, an ethyl group, an n-propyl group, an isopropyl group, an n-butyl group, an isobutyl group, an s-butyl group, or a t-butyl group, more preferably a methyl group, an ethyl group, an isopropyl group, or a t-butyl group, even more preferably a methyl group or a t-butyl group.

Details of the substituted or unsubstituted cycloalkyl group having 3 to 50 ring carbon atoms are as described in the section of “substituents described in the present specification”.

The unsubstituted cycloalkyl group is preferably a cyclopropyl group, a cyclobutyl group, a cyclopentyl group, a cyclohexyl group, a 1-adamantyl group, a 2-adamantyl group, a 1-norbornyl group, or a 2-norbornyl group, more preferably a cyclopropyl group, a cyclobutyl group, a cyclopentyl group or a cyclohexyl group, even more preferably a cyclopentyl group or a cyclohexyl group.

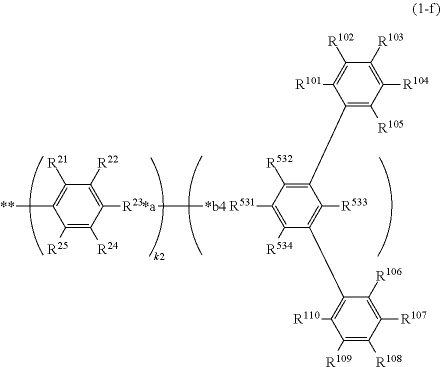

In the formula (1-f):

** indicates a bonding position boning to the central nitrogen atom N*.

R²¹ to R²⁵, R⁵³¹ to R⁵³⁴ and R¹⁰¹ to R¹¹⁰ each independently represent a hydrogen atom or the substituent A.

k2 represents 0 or 1, provide that:
when k2 is 0, *a indicates a bonding position bonding to the central nitrogen atom N*,
when k2 is 1, one selected from R²¹ to R²⁵ is a single bond bonding to *a, and one selected from R⁵³¹ to R⁵³⁴ is a single bond bonding to *b4.

R²¹ to R²⁵ not being a single bond, and R⁵³¹ to R⁵³⁴, R¹⁰¹ to R¹⁰⁵, and R¹⁰⁶ to R¹¹⁰ not being a single bond do not bond to each other and therefore do not form a cyclic structure.

R¹⁰⁶ to R¹¹⁰, R¹⁰¹ to R¹⁰⁵, R²¹ to R²⁵ not being a single bond, and R⁵³¹ to R⁵³⁴ not being a single bond are each independently preferably a hydrogen atom, a halogen atom, a cyano group, a substituted or unsubstituted alkyl group having 1 to 50 carbon atoms, or a substituted or unsubstituted cycloalkyl group having 3 to 50 ring carbon atoms, more preferably a hydrogen atom or a substituted or unsubstituted alkyl group having 1 to 50 carbon atoms.

Details of the halogen atom are as described in the section of “substituents described in the present specification”.

Details of the substituted or unsubstituted alkyl group having 1 to 50 carbon atoms are as described in the section of “substituents described in the present specification”.

The unsubstituted alkyl group is preferably a methyl group, an ethyl group, an n-propyl group, an isopropyl group, an n-butyl group, an isobutyl group, an s-butyl group, or a t-butyl group, more preferably a methyl group, an ethyl group, an isopropyl group, or a t-butyl group, even more preferably a methyl group or a t-butyl group.

Details of the substituted or unsubstituted cycloalkyl group having 3 to 50 ring carbon atoms are as described in the section of “substituents described in the present specification”.

The unsubstituted cycloalkyl group is preferably a cyclopropyl group, a cyclobutyl group, a cyclopentyl group, a cyclohexyl group, a 1-adamantyl group, a 2-adamantyl group, a 1-norbornyl group, or a 2-norbornyl group, more preferably a cyclopropyl group, a cyclobutyl group, a cyclopentyl group or a cyclohexyl group, even more preferably a cyclopentyl group or a cyclohexyl group.

In one aspect of the formula (1-f), k2 is 1, R²¹, R²², or R²³ is a single bond bonding to *a, and R⁵³¹ or R⁵³² is a single bond bonding to *b4.

The compound represented by the formula (1) is preferably represented by the following formula (1-1a):

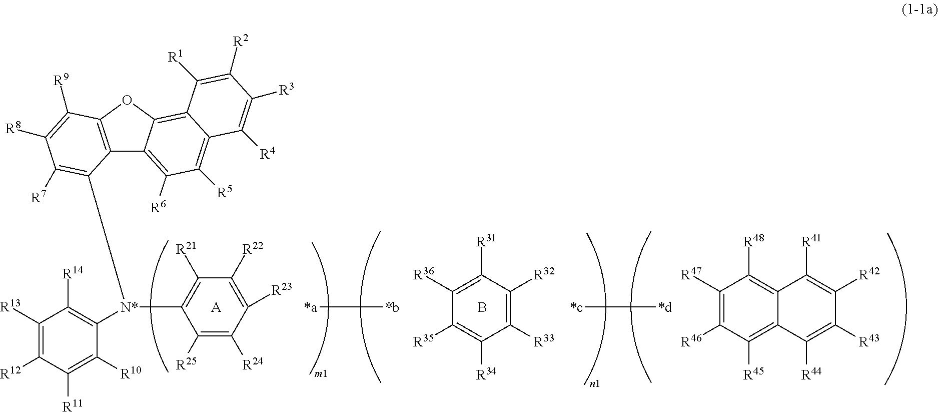

In the formula (1-1a), N*, R¹ to R¹⁴, R²¹ to R²⁵, R³¹ to R³⁶, R⁴¹ to R⁴⁸, *a, *b, *c, *d, m1, and n1 are as defined in the formula (1), and details of preferred groups are as described for the formula (1).

The compound represented by the formula (1) is preferably represented by the formula (1-1b):

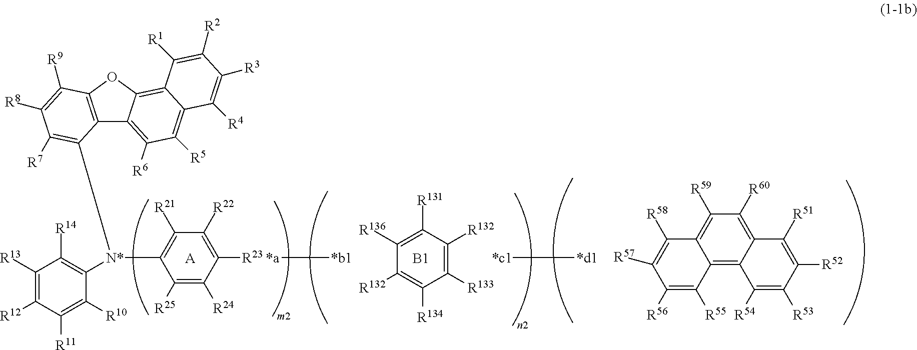

In the formula (1-1b), N*, R¹ to R¹⁴, R²¹ to R²⁵, R⁵¹ to R⁶⁰, R¹³¹ to R¹³⁶, *a, *b1, *c1, *d1, m2, and n2 are as defined in the formula (1), and details of preferred groups are as described for the formula (1).

Also, the compound represented by the formula (1) is preferably represented by the following formula (1-1c):

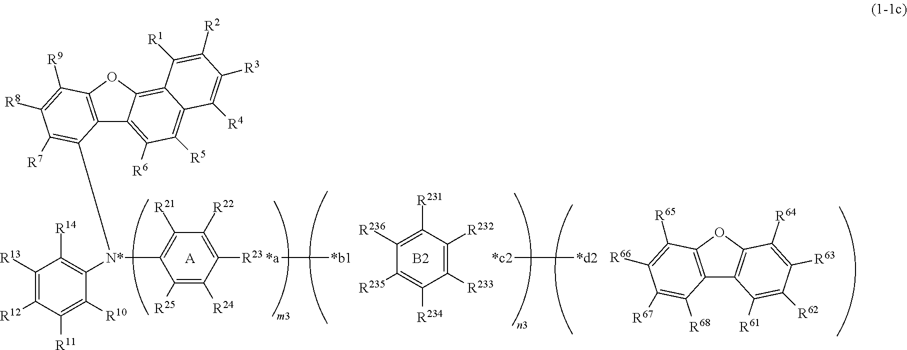

In the formula (1-1c), N*, R¹ to R¹⁴, R²¹ to R²⁵, R⁶¹ to R⁶⁸, R²³¹ to R²³⁶, X, *a, *b2, *c2, *d2, m3, and n3 are as defined in the formula (1), and details of preferred groups are as described for the formula (1).

Also, the compound represented by the formula (1) is preferably represented by the following formula (1-1d):

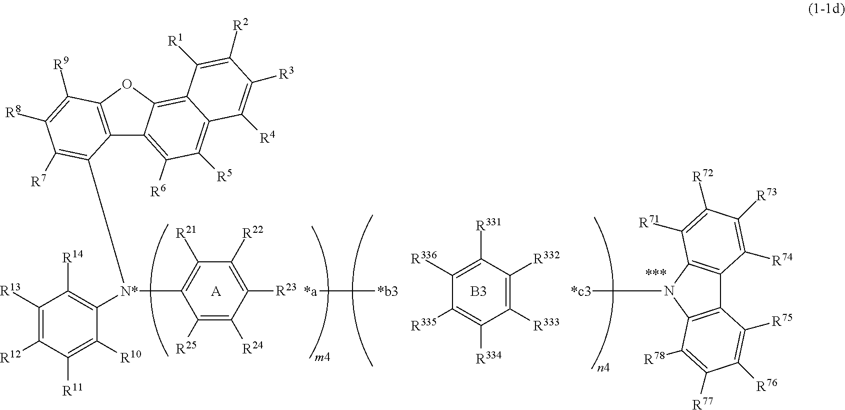

In the formula (1-1d), N*, N***, R¹ to R¹⁴, R²¹ to R²⁵, R⁷¹ to R⁷⁸, R³³¹ to R³³⁶, *a, *b3, *c3, m4, and n4 are as defined in the formula (1), and details of preferred groups are as described for the formula (1).

Also, the compound represented by the formula (1) is preferably represented by the following formula (1-1e):

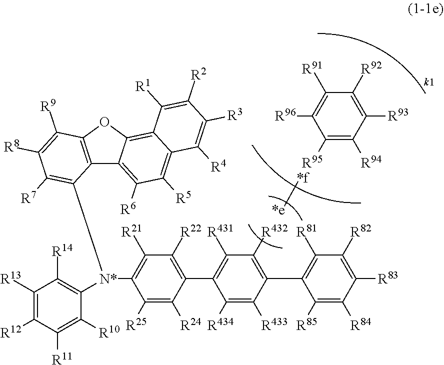

In the formula (1-1e), N*, R¹ to R¹⁴, R²¹, R²², R²⁴, R²⁵, R⁸¹ to R⁸⁵, R⁹¹ to R⁹⁶, R⁴³¹ to R⁴³⁴, *e, *f, and k1 are as defined in the formula (1), and details of preferred groups are as described for the formula (1).

Also, the compound represented by the formula (1) is preferably represented by the following formula (1-1f):

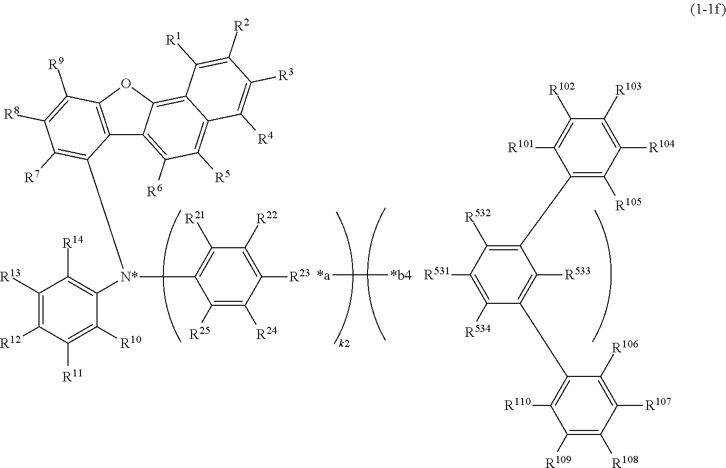

In the formula (1-1f), N*, R¹ to R¹⁴, R²¹ to R²⁵, R¹⁰¹ to R¹¹⁰, R⁵³¹ to R⁵³⁴, *a, *b4, and k2 are as defined in the formula (1), and details of preferred groups are as described for the formula (1).

Also, the compound represented by the formula (1) is preferably represented by the following formula (1-1a-1):

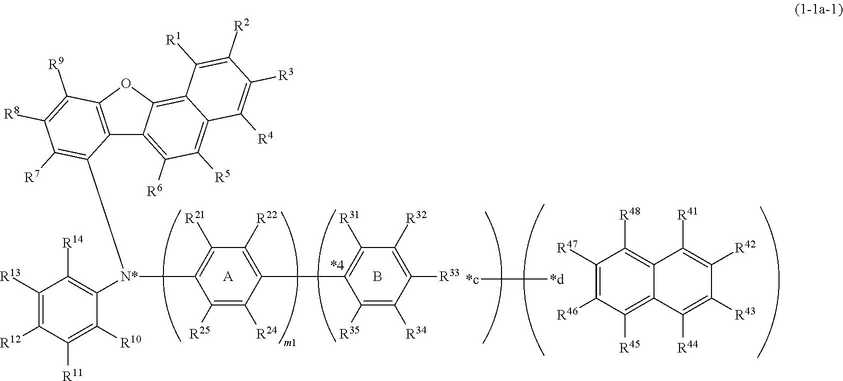

In the formula (1-1a-1):
m1 represents 0 or 1,
when m1 is 0, the carbon^{*4} bonds to the central nitrogen atom N*.

One selected from R³¹ to R³⁵ is a single bond bonding to *c.

N*, R¹ to R¹⁴, R²¹ to R²², R²⁴ to R²⁵, R⁴¹ to R⁴⁸, R³¹ to R³⁵ not being a single bond bonding to *c, *c and *d are as defined in the formula (1), and details of preferred groups are as described for the formula (1).

Also, the compound represented by the formula (1) is preferably represented by the following formula (1-1b-1):

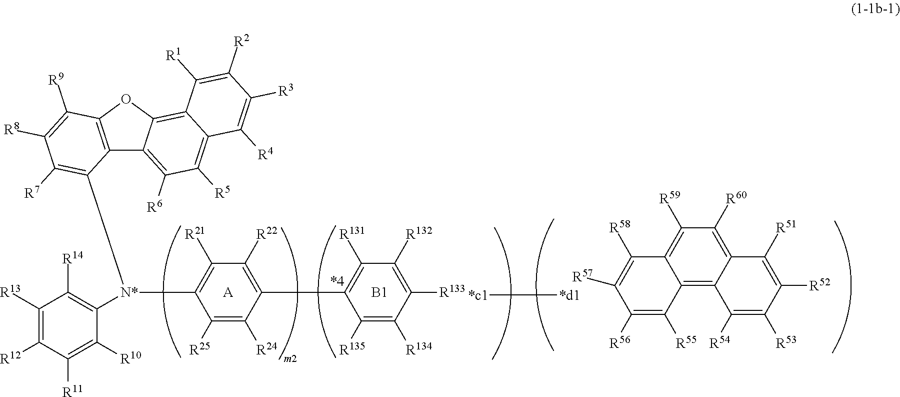

In the formula (1-1b-1):
m2 represents 0 or 1,
when m2 is 0, the carbon^{*4} bonds to the central nitrogen atom N*.

One selected from R¹³¹ to R¹³⁵ is a single bond bonding to *c1.

N*, R¹ to R¹⁴, R²¹ to R²², R²⁴ to R²⁵, R⁵¹ to R⁶⁰, R¹³¹ to R¹³⁵ not being a single bond bonding to *c1, *c1, and *d1 are as defined in the formula (1), and details of preferred groups are as described for the formula (1).

Also, the compound represented by the formula (1) is preferably represented by the following formula (1-1c-1):

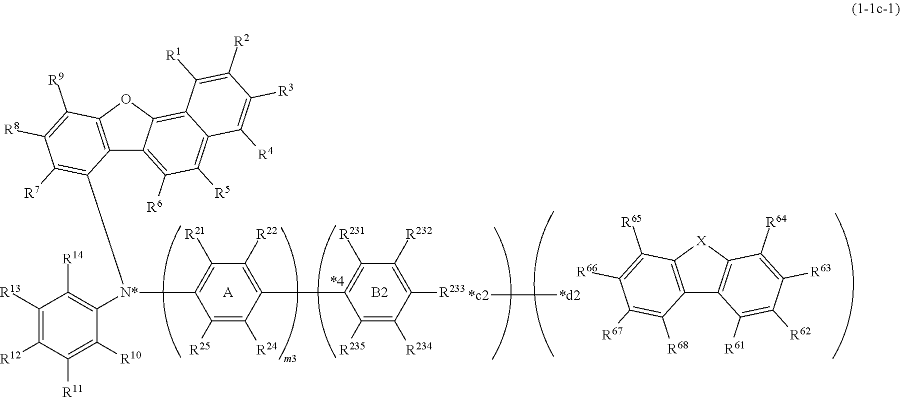

In the formula (1-1c-1):
m3 represents 0 or 1,
when m3 is 0, the carbon^{*4} bonds to the central nitrogen atom N*.

One selected from R²³¹ to R²³⁵ is a single bond bonding to *c2.

N*, R¹ to R¹⁴, R²¹ to R²², R²⁴ to R²⁵, R⁶¹ to R⁶⁸, R²³¹ to R²³⁵ not being a single bond bonding to *c2, X, *c2, and *d2 are as defined in the formula (1), and details of preferred groups are as described for the formula (1).

Also, the compound represented by the formula (1) is preferably represented by the following formula (1-1d-1):

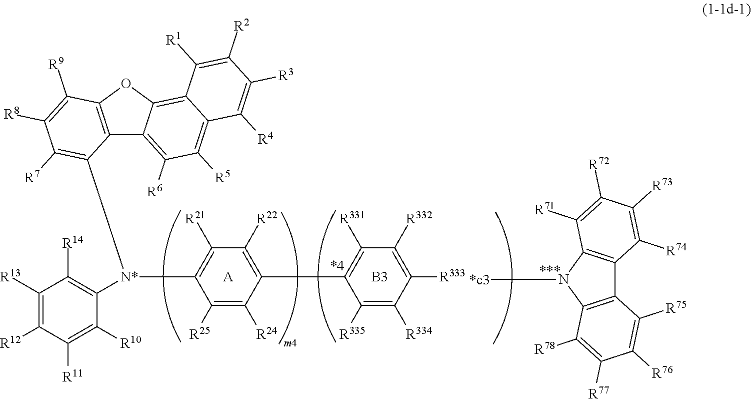

In the formula (1-1d-1):
m4 represents 0 or 1,
when m4 is 0, the carbon^{*4} bonds to the central nitrogen atom N*.

One selected from R³³¹ to R³³⁵ is a single bond bonding to *c3.

N*, N***, R¹ to R¹⁴, R²¹ to R²², R²⁴ to R²⁵, R⁷¹ to R⁷⁸, R³³¹ to R³³⁵ not being a single bond bonding to *c3, and *c3 are as defined in the formula (1), and details of preferred groups are as described for the formula (1).

Also, the compound represented by the formula (1) is preferably represented by the following formula (1-1a-2):

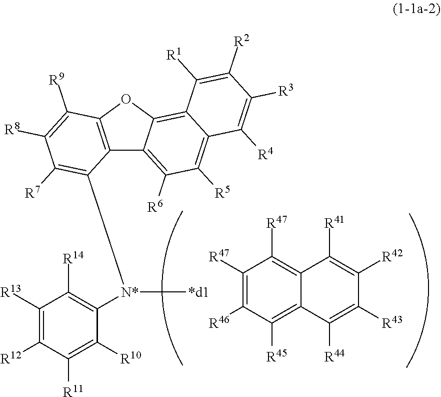

In the formula (1-1a-2) N*, R¹ to R¹⁴, R⁴¹ to R⁴⁸, and *d are as defined in the formula (1), and details of preferred groups are as described for the formula (1).

Also, the compound represented by the formula (1) is preferably represented by the following formula (1-1b-2):

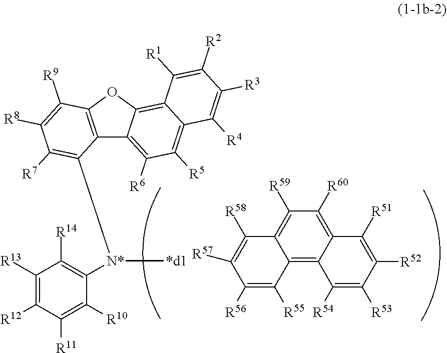

In the formula (1-1b-2), N*, R¹ to R¹⁴, R⁵¹ to R⁶⁰, X, and *d1 are as defined in the formula (1), and details of preferred groups are as described for the formula (1).

Also, the compound represented by the formula (1) is preferably represented by the following formula (1-2c-2):

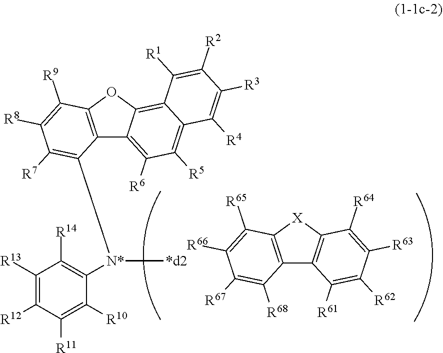

In the formula (1-1c-2), N*, R¹ to R¹⁴, R⁶¹ to R⁶⁸, X, and *d2 are as defined in the formula (1), and details of preferred groups are as described for the formula (1).

Also, the compound represented by the formula (1) is preferably represented by the following formula (1-1f-1):

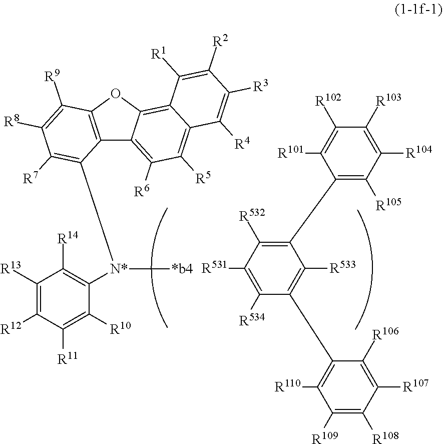

In the formula (1-1f-1), N*, R¹ to R¹⁴, R¹⁰¹ to R¹¹⁰, R⁵³¹ to R⁵³⁴ and *b4 are as defined in the formula (1), and details of preferred groups are as described for the formula (1).

Also, the compound represented by the formula (1) is preferably represented by the following formula (1-1a-3), formula (1-1a-4) or formula (1-1a-5):

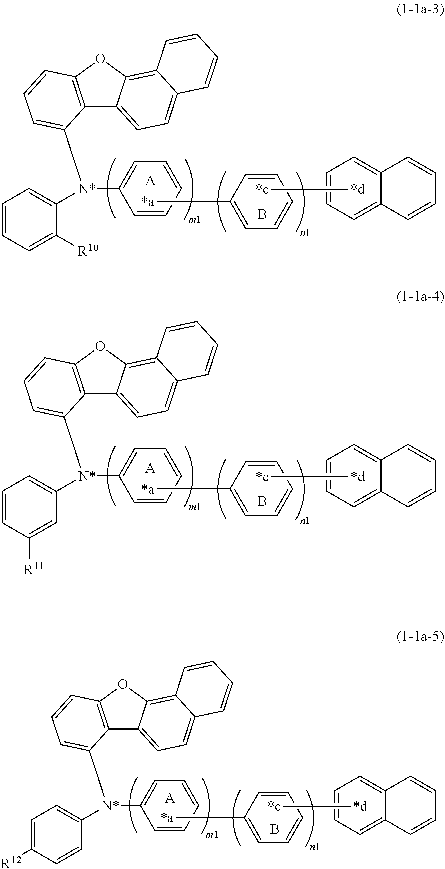

In the formula (1-1a-3), the formula (1-1a-4) and the formula (1-1a-5), R¹¹ to R¹³, *a, *c, *d, m1, and n1 are as defined in the formula (1), and details of preferred groups are as described for the formula (1).

Also, the compound represented by the formula (1) is preferably represented by the following formula (1-1b-3), formula (1-1b-4) or formula (1-1b-5):

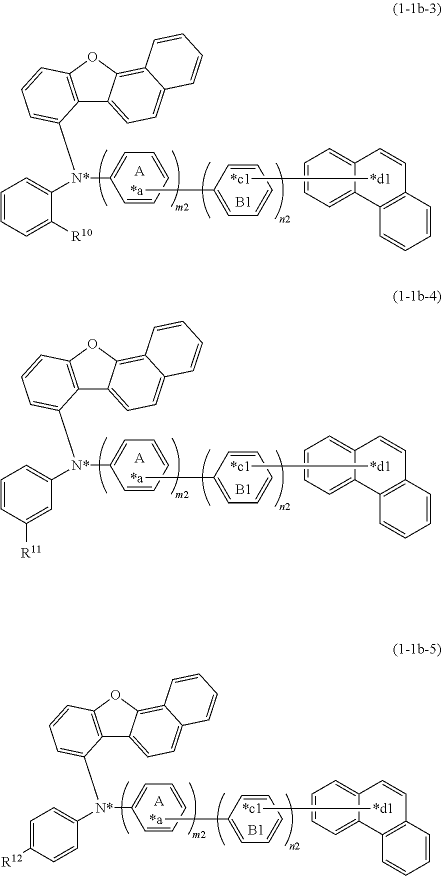

In the formula (1-1b-3), the formula (1-1b-4) and the formula (1-1b-5), R¹¹ to R¹³, *a, *c1, *d1, m2, and n2 are as defined in the formula (1), and details of preferred groups are as described for the formula (1).

Also, the compound represented by the formula (1) is preferably represented by the following formula (1-1c-3), formula (1-1c-4) or formula (1-1c-5):

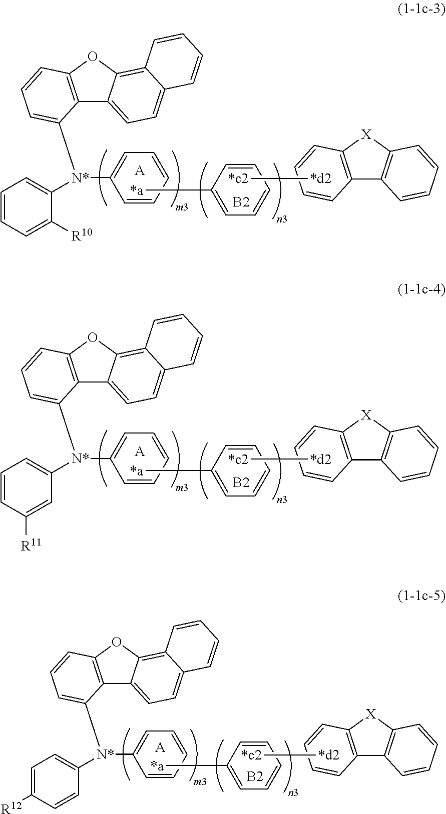

In the formula (1-1c-3), the formula (1-1c-4) and the formula (1-1c-5), N*, R¹¹ to R¹³, *a, *c2, *d2, m3, and n3 are as defined in the formula (1), and details of preferred groups are as described for the formula (1).

Also, the compound represented by the formula (1) is preferably represented by the following formula (1-1d-3), formula (1-1d-4) or formula (1-1d-5):

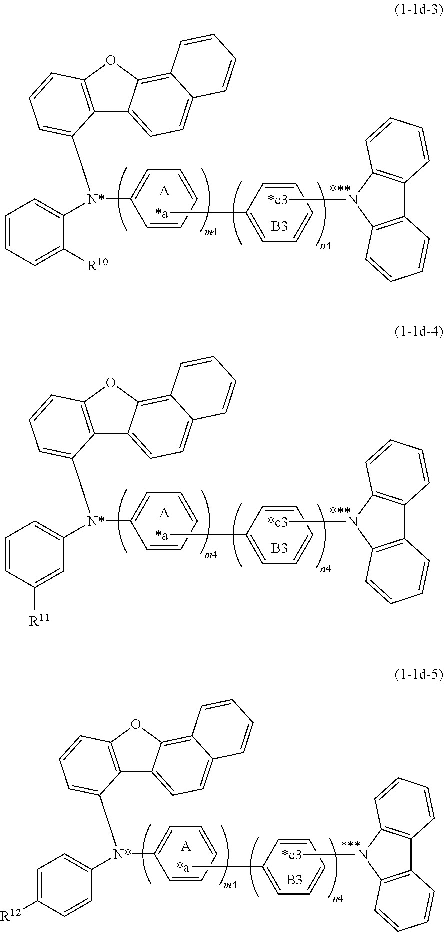

In the formula (1-1d-3), the formula (1-1d-4) and the formula (1-1d-5), N*, N***, to R¹³, *a, *c3, m4, and n4 are as defined in the formula (1), and details of preferred groups are as described for the formula (1).

Also, the compound represented by the formula (1) is preferably represented by the following formula (1-1e-1), formula (1-1e-2) or formula (1-1e-3):

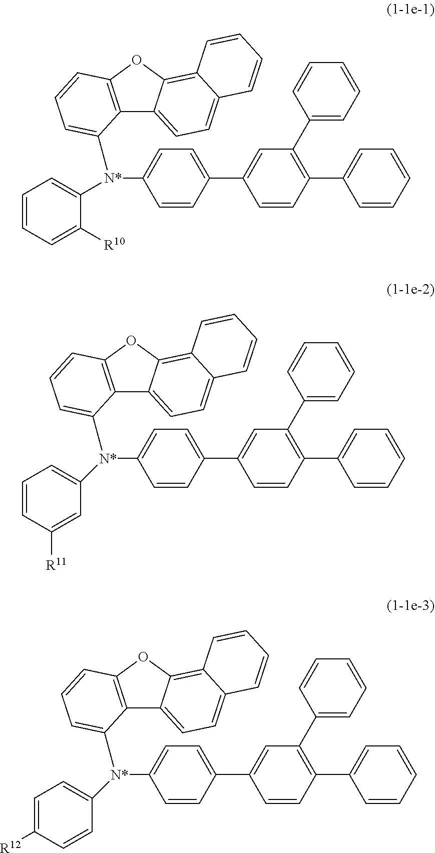

In the formula (1-1e-1), the formula (1-1e-2) and the formula (1-1e-3), N*, and R¹¹ to R¹³ are as defined in the formula (1), and details of preferred groups are as described for the formula (1).

Also, in one aspect, the compound represented by the formula (1) is preferably represented by the following formula (1-1e-4), formula (1-1e-5) or formula (1-1e-6):

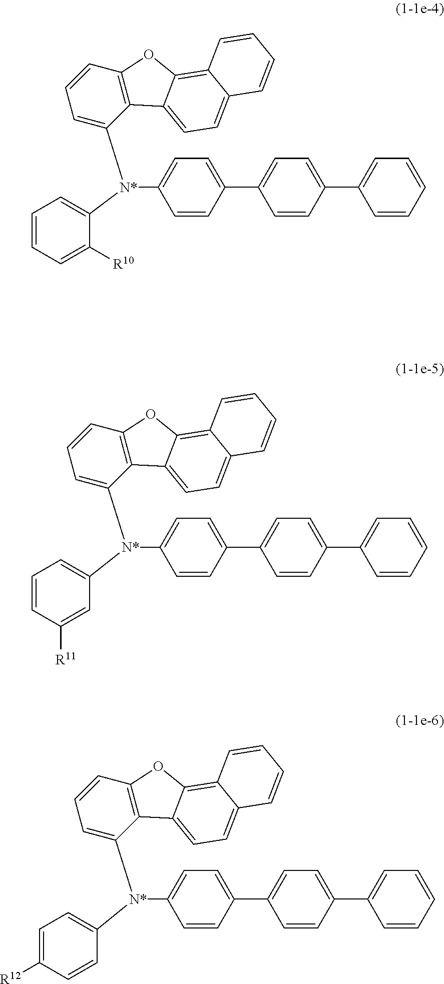

In the formula (1-1e-4), the formula (1-1e-5) and the formula (1-1e-6), N*, and R¹¹ to R¹³ are as defined in the formula (1), and details of preferred groups are as described for the formula (1).

Also, the compound represented by the formula (1) is preferably represented by the following formula (1-1f-3), formula (1-1f-4) or formula (1-1f-5):

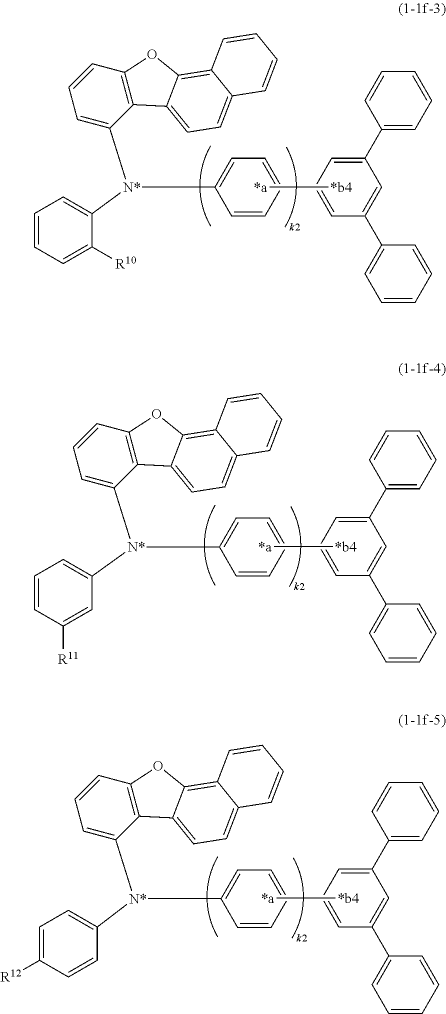

In the formula (1-1f-3), the formula (1-1f-4) and the formula (1-1f-5), N*, R¹¹ to R¹³, *a, *b4, and k2 are as defined in the formula (1), and details of preferred groups are as described for the formula (1).

In one aspect of the present invention:
(1-1) all R¹ to R⁹ can be hydrogen atoms,
(1-2) all R¹⁰ to R¹⁴ except an aryl group and a heterocyclic group can be hydrogen atoms,
(1-3) the substituted or unsubstituted aryl group having 6 to 30 ring carbon atoms, which R¹⁰ to R¹⁴ represent, can be unsubstituted,
(1-4) the substituted or unsubstituted heterocyclic group having 5 to 30 ring atoms, which R¹⁰ to R¹⁴ represent, can be unsubstituted,
(1-5) all R²¹ to R²⁵ not being a single bond bonding to *a can be hydrogen atoms,
(1-6) all R³¹ to R³⁶ not being a single bond bonding to *b, and not being a single bond bonding to *c can be hydrogen atoms,
(1-7) all R⁴¹ to R⁴⁸ not being a single bond bonding to *d can be hydrogen atoms,
(1-8) all R¹³¹ to R¹³⁶ not being a single bond bonding to *b1, and not being a single bond bonding to *c1 can be hydrogen atoms,
(1-9) all R⁵¹ to R⁶⁰ not being a single bond bonding to *d1 can be hydrogen atoms,
(1-10) all R²³¹ to R²³⁶ not being a single bond bonding to *b2, and not being a single bond bonding to *c2 can be hydrogen atoms,
(1-11) all R⁶¹ to R⁶⁸ not being a single bond bonding to *d2 can be hydrogen atoms,
(1-12) the benzene ring formed by adjacent one or more pairs of R⁶¹ to R⁶⁸ can be unsubstituted, and all the other R⁶¹ to R⁶⁸ not being a single bond bonding to *d2 can be hydrogen atoms,
(1-13) all R³³¹ to R³³⁶ not being a single bond bonding to *b3, and not being a single bond bonding to *c3 can be hydrogen atoms,
(1-14) all R⁷¹ to R⁷⁸ can be hydrogen atoms,
(1-15) all R⁴³¹, R⁴³³, R⁴³⁴, and R⁴³² not being a single bond bonding to *e can be hydrogen atoms,
(1-16) all R⁹¹ to R⁹¹ not being a single bond bonding to *f can be hydrogen atoms,
(1-17) all R⁸¹ to R⁸⁵ can be hydrogen atoms,
(1-18) all R⁵³¹ to R⁵³⁴ not being a single bond bonding to *b4 can be hydrogen atoms,
(1-19) all R¹⁰¹ to R¹¹⁰ can be hydrogen atoms.

As described above, the “hydrogen atom” referred to in the present specification encompasses a protium atom, a deuterium atom, and tritium atom. Accordingly, the compound (1) can contain a naturally-derived deuterium atom.

A deuterium atom can be intentionally introduced into the compound (1) by using a deuterated compound as a part or the whole of the raw material. Accordingly, in one aspect of the present invention, the compound (1) contains at least one deuterium atom. That is, the inventive compound can be the compound represented by the formula (1) or by other formulae in which at least one hydrogen atom contained in the compound is a deuterium atom.

At least one hydrogen atom selected from the following hydrogen atoms can be a deuterium atom:
a hydrogen atom that any of R¹ to R⁹ represents; a hydrogen atom of the substituted or unsubstituted alkyl group, the substituted or unsubstituted alkenyl group, the substituted or unsubstituted alkynyl group, the substituted or unsubstituted cycloalkyl group, the substituted or unsubstituted haloalkyl group, the substituted or unsubstituted alkoxy group, the substituted or unsubstituted haloalkoxy group, the substituted or unsubstituted alkylthio group, the substituted or unsubstituted aryloxy group, the substituted or unsubstituted arylthio group, the substituted or unsubstituted aralkyl group, or the mono, di or tri-substituted silyl group that any of R¹ to R⁹ represents;
a hydrogen atom that any of R²¹ to R²⁶ represents; a hydrogen atom of the substituted or unsubstituted alkyl group, the substituted or unsubstituted alkenyl group, the substituted or unsubstituted alkynyl group, the substituted or unsubstituted cycloalkyl group, the substituted or unsubstituted haloalkyl group, the substituted or unsubstituted alkoxy group, the substituted or unsubstituted haloalkoxy group, the substituted or unsubstituted alkylthio group, the substituted or unsubstituted aryloxy group, the substituted or unsubstituted arylthio group, the substituted or unsubstituted aralkyl group, or the mono, di or tri-substituted silyl group that any of R²¹ to R²⁶ represents;
a hydrogen atom that any of R³¹ to R³⁶ represents; a hydrogen atom of the substituted or unsubstituted alkyl group, the substituted or unsubstituted alkenyl group, the substituted or unsubstituted alkynyl group, the substituted or unsubstituted cycloalkyl group, the substituted or unsubstituted haloalkyl group, the substituted or unsubstituted alkoxy group, the substituted or unsubstituted haloalkoxy group, the substituted or unsubstituted alkylthio group, the substituted or unsubstituted aryloxy group, the substituted or unsubstituted arylthio group, the substituted or unsubstituted aralkyl group, or the mono, di or tri-substituted silyl group that any of R³¹ to R³⁶ represents;
a hydrogen atom that any of R⁴¹ to R⁴⁸ represents; a hydrogen atom of the phenyl group, the substituted or unsubstituted alkyl group, the substituted or unsubstituted alkenyl group, the substituted or unsubstituted alkynyl group, the substituted or unsubstituted cycloalkyl group, the substituted or unsubstituted haloalkyl group, the substituted or unsubstituted alkoxy group, the substituted or unsubstituted haloalkoxy group, the substituted or unsubstituted alkylthio group, the substituted or unsubstituted aryloxy group, the substituted or unsubstituted arylthio group, the substituted or unsubstituted aralkyl group, or the mono, di or tri-substituted silyl group that any of R⁴¹ to R⁴⁸ represents;
a hydrogen atom that any of R⁵¹ to R⁶⁰ represents; a hydrogen atom of the phenyl group, the substituted or unsubstituted alkyl group, the substituted or unsubstituted alkenyl group, the substituted or unsubstituted alkynyl group, the substituted or unsubstituted cycloalkyl group, the substituted or unsubstituted haloalkyl group, the substituted or unsubstituted alkoxy group, the substituted or unsubstituted haloalkoxy group, the substituted or unsubstituted alkylthio group, the substituted or unsubstituted aryloxy group, the substituted or unsubstituted arylthio group, the substituted or unsubstituted aralkyl group, or the mono, di or tri-substituted silyl group that any of R⁵¹ to R⁶⁰ represents;
a hydrogen atom that any of R⁶¹ to R⁶⁸ represents; a hydrogen atom of the phenyl group, the substituted or unsubstituted alkyl group, the substituted or unsubstituted alkenyl group, the substituted or unsubstituted alkynyl group, the substituted or unsubstituted cycloalkyl group, the substituted or unsubstituted haloalkyl group, the substituted or unsubstituted alkoxy group, the substituted or unsubstituted haloalkoxy group, the substituted or unsubstituted alkylthio group, the substituted or unsubstituted aryloxy group, the substituted or unsubstituted arylthio group, the substituted or unsubstituted aralkyl group, or the mono, di or tri-substituted silyl group that any of R⁶¹ to R⁶⁸ represents; a hydrogen atom of the substituted or unsubstituted benzene ring formed by adjacent one or more pairs of R⁶¹ to R⁶⁸;
a hydrogen atom that any of R⁷¹ to R⁷⁸ represents; a hydrogen atom of the phenyl group, the substituted or unsubstituted alkyl group, the substituted or unsubstituted alkenyl group, the substituted or unsubstituted alkynyl group, the substituted or unsubstituted cycloalkyl group, the substituted or unsubstituted haloalkyl group, the substituted or unsubstituted alkoxy group, the substituted or unsubstituted haloalkoxy group, the substituted or unsubstituted alkylthio group, the substituted or unsubstituted aryloxy group, the substituted or unsubstituted arylthio group, the substituted or unsubstituted aralkyl group, or the mono, di or tri-substituted silyl group that any of R⁷¹ to R⁷⁸ represents;
a hydrogen atom that any of R⁸¹ to R⁸⁵ represents; a hydrogen atom of the substituted or unsubstituted alkyl group, the substituted or unsubstituted alkenyl group, the substituted or unsubstituted alkynyl group, the substituted or unsubstituted cycloalkyl group, the substituted or unsubstituted haloalkyl group, the substituted or unsubstituted alkoxy group, the substituted or unsubstituted haloalkoxy group, the substituted or unsubstituted alkylthio group, the substituted or unsubstituted aryloxy group, the substituted or unsubstituted arylthio group, the substituted or unsubstituted aralkyl group, or the mono, di or tri-substituted silyl group that any of R⁸¹ to R⁸⁵ represents;
a hydrogen atom that any of R⁹¹ to R⁹⁶ represents; a hydrogen atom of the substituted or unsubstituted alkyl group, the substituted or unsubstituted alkenyl group, the substituted or unsubstituted alkynyl group, the substituted or unsubstituted cycloalkyl group, the substituted or unsubstituted haloalkyl group, the substituted or unsubstituted alkoxy group, the substituted or unsubstituted haloalkoxy group, the substituted or unsubstituted alkylthio group, the substituted or unsubstituted aryloxy group, the substituted or unsubstituted arylthio group, the substituted or unsubstituted aralkyl group, or the mono, di or tri-substituted silyl group that any of R⁹¹ to R⁹⁶ represents;
a hydrogen atom that any of R¹⁰¹ to R¹¹⁰ represents; a hydrogen atom of the substituted or unsubstituted alkyl group, the substituted or unsubstituted alkenyl group, the substituted or unsubstituted alkynyl group, the substituted or unsubstituted cycloalkyl group, the substituted or unsubstituted haloalkyl group, the substituted or unsubstituted alkoxy group, the substituted or unsubstituted haloalkoxy group, the substituted or unsubstituted alkylthio group, the substituted or unsubstituted aryloxy group, the substituted or unsubstituted arylthio group, the substituted or unsubstituted aralkyl group, or the mono, di or tri-substituted silyl group that any of R¹⁰¹ to R¹¹⁰ represents;
a hydrogen atom that any of R¹³¹ to R¹³⁶ represents; a hydrogen atom of the substituted or unsubstituted alkyl group, the substituted or unsubstituted alkenyl group, the substituted or unsubstituted alkynyl group, the substituted or unsubstituted cycloalkyl group, the substituted or unsubstituted haloalkyl group, the substituted or unsubstituted alkoxy group, the substituted or unsubstituted haloalkoxy group, the substituted or unsubstituted alkylthio group, the substituted or unsubstituted aryloxy group, the substituted or unsubstituted arylthio group, the substituted or unsubstituted aralkyl group, or the mono, di or tri-substituted silyl group that any of R¹³¹ to R¹³⁶ represents;
a hydrogen atom that any of R²³¹ to R²³⁶ represents; a hydrogen atom of the substituted or unsubstituted alkyl group, the substituted or unsubstituted alkenyl group, the substituted or unsubstituted alkynyl group, the substituted or unsubstituted cycloalkyl group, the substituted or unsubstituted haloalkyl group, the substituted or unsubstituted alkoxy group, the substituted or unsubstituted haloalkoxy group, the substituted or unsubstituted alkylthio group, the substituted or unsubstituted aryloxy group, the substituted or unsubstituted arylthio group, the substituted or unsubstituted aralkyl group, or the mono, di or tri-substituted silyl group that any of R²³¹ to R²³⁶ represents;
a hydrogen atom that any of R³³¹ to R³³⁶ represents; a hydrogen atom of the substituted or unsubstituted alkyl group, the substituted or unsubstituted alkenyl group, the substituted or unsubstituted alkynyl group, the substituted or unsubstituted cycloalkyl group, the substituted or unsubstituted haloalkyl group, the substituted or unsubstituted alkoxy group, the substituted or unsubstituted haloalkoxy group, the substituted or unsubstituted alkylthio group, the substituted or unsubstituted aryloxy group, the substituted or unsubstituted arylthio group, the substituted or unsubstituted aralkyl group, or the mono, di or tri-substituted silyl group that any of R³³¹ to R³³⁶ represents;
a hydrogen atom that any of R⁴³¹ to R⁴³⁴ represents; a hydrogen atom of the substituted or unsubstituted alkyl group, the substituted or unsubstituted alkenyl group, the substituted or unsubstituted alkynyl group, the substituted or unsubstituted cycloalkyl group, the substituted or unsubstituted haloalkyl group, the substituted or unsubstituted alkoxy group, the substituted or unsubstituted haloalkoxy group, the substituted or unsubstituted alkylthio group, the substituted or unsubstituted aryloxy group, the substituted or unsubstituted arylthio group, the substituted or unsubstituted aralkyl group, or the mono, di or tri-substituted silyl group that any of R⁴³¹ to R⁴³⁴ represents;
a hydrogen atom that any of R⁵³¹ to R⁵³⁴ represents; a hydrogen atom of the substituted or unsubstituted alkyl group, the substituted or unsubstituted alkenyl group, the substituted or unsubstituted alkynyl group, the substituted or unsubstituted cycloalkyl group, the substituted or unsubstituted haloalkyl group, the substituted or unsubstituted alkoxy group, the substituted or unsubstituted haloalkoxy group, the substituted or unsubstituted alkylthio group, the substituted or unsubstituted aryloxy group, the substituted or unsubstituted arylthio group, the substituted or unsubstituted aralkyl group, or the mono, di or tri-substituted silyl group that any of R⁵³¹ to R⁵³⁴ represents;
a hydrogen atom that any of R¹⁰ to R¹⁴ represents; a hydrogen atom of the substituted or unsubstituted aryl group having 6 to 30 ring carbon atoms that any of R¹⁰ to R¹⁴ represents; a hydrogen atom of the substituted or unsubstituted group having 5 to 30 ring atoms that any of R¹⁰ to R¹⁴ represents; and
a hydrogen atom that R^{a} represents; a hydrogen atom of the substituted or unsubstituted alkyl group having 1 to 50 carbon atoms, or the substituted or unsubstituted aryl group having 6 to 50 ring carbon atoms that R^{a} represents.

The deuteration rate of the compound (1) depends on the deuteration rate of the raw material compound used. Even though a raw material having a predetermined deuteration rate is used, it may contain a naturally-derived protium isotope in a certain ratio. Accordingly, an aspect of the deuteration rate of the compound (1) shown below contains a ratio for which a naturally-derived minor isotope is taken into consideration relative to the ratio determined merely by counting the number of the deuterium atoms represented by a chemical formula.

The deuteration rate of the compound (1) is preferably 1% or more, more preferably 3% or more, even more preferably 5% or more, further more preferably 10% or more, further more preferably 50% or more.

The compound (1) can be a mixture of a deuterated compound and a non-deuterated compound, or a mixture of two or more compounds having different deuteration rates from each other. The deuteration rate of the mixture is preferably 1% or more, more preferably 3% or more, even more preferably 5% or more, further more preferably 10% or more, further more preferably 50% or more, and is less than 100%.

The ratio of the number of the deuterium atoms relative to the number of all hydrogen atoms in the compound (1) is preferably 1% or more, more preferably 3% or more, even more preferably 5% or more, further more preferably 10% or more, and is 100% or less.

Details of the substituent (arbitrary substituent) in the case of “substituted or unsubstituted” contained in the definition of the above-mentioned formulae are as described in the section of “substituent in the case of “substituted or unsubstituted””.

The arbitrary substituent contained in the definition for the above-mentioned formulae does not contain the aryl group, the heterocyclic group and the substituent represented by —N(R⁹⁰⁶)(R⁹⁰⁷) among the substituents described in the section of “substituent in the case of “substituted or unsubstituted””.

The arbitrary substituent contained in the definition of R²¹ to R²⁵ not being a single bond bonding to *a; R³¹ to R³⁶ not being a single bond bonding to *b and not being a single bond bonding to *c; R¹³¹ to R¹³⁶ not being a single bond bonding to *b1 and not being a single bond bonding to *c1; R²³¹ to R²³⁶ not being a single bond bonding to *b2 and not being a single bond bonding to *c2; R³³¹ to R³³⁶ not being a single bond bonding to *b3 and not being a single bond bonding to *c3; R⁴³² not being a single bond bonding to *e; R⁴³¹, R⁴³³, and R⁴³⁴; R⁸¹ to R⁸⁵; R⁹¹ to R⁹⁶ not being a single bond bonding to f; R¹⁰¹ to R¹¹⁰; and R⁵³¹ to R⁵³⁴ not being a single bond bonding to *b4 does not contain the aryl group, the heterocyclic group and the substituent represented by —N(R⁹⁰⁶)(R⁹⁰⁷) among the substituents described in the section of “substituent in the case of “substituted or unsubstituted””.

The inventive compound can be readily produced by a person skilled in the art with reference to the following synthesis examples and known synthesis methods.

Specific examples of the inventive compound will be described below, but the inventive compound is not limited to the following example compounds.

In the following specific examples, D represents a deuterium atom.

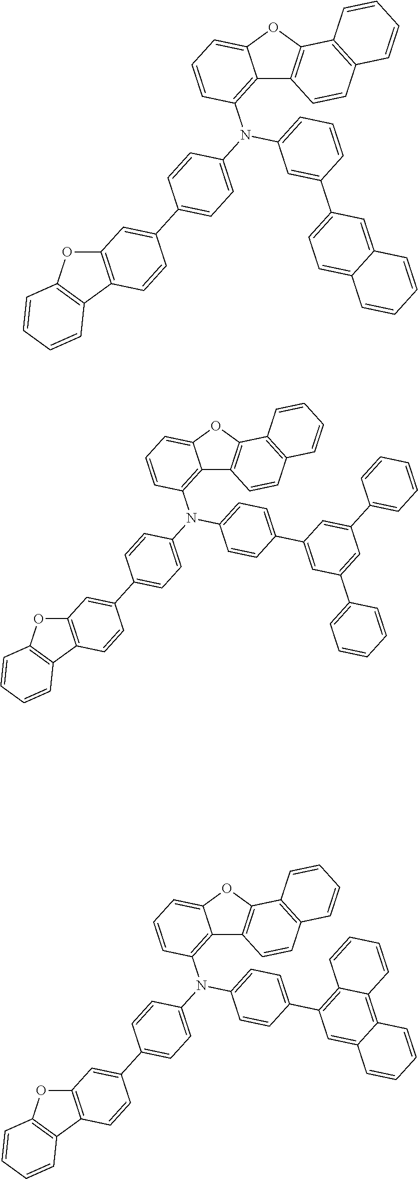 [SMILES: c1cc(-c2ccc3ccccc3c2)cc(N(c2ccc(-c3ccc4c(c3)oc3ccccc34)cc2)c2cccc3oc4c5ccccc5ccc4c23)c1 ; c1ccc(-c2cc(-c3ccccc3)cc(-c3ccc(N(c4ccc(-c5ccc6c(c5)oc5ccccc56)cc4)c4cccc5oc6c7ccccc7ccc6c45)cc3)c2)cc1 ; c1ccc2c(c1)cc(-c1ccc(N(c3ccc(-c4ccc5c(c4)oc4ccccc45)cc3)c3cccc4oc5c6ccccc6ccc5c34)cc1)c1ccccc12]

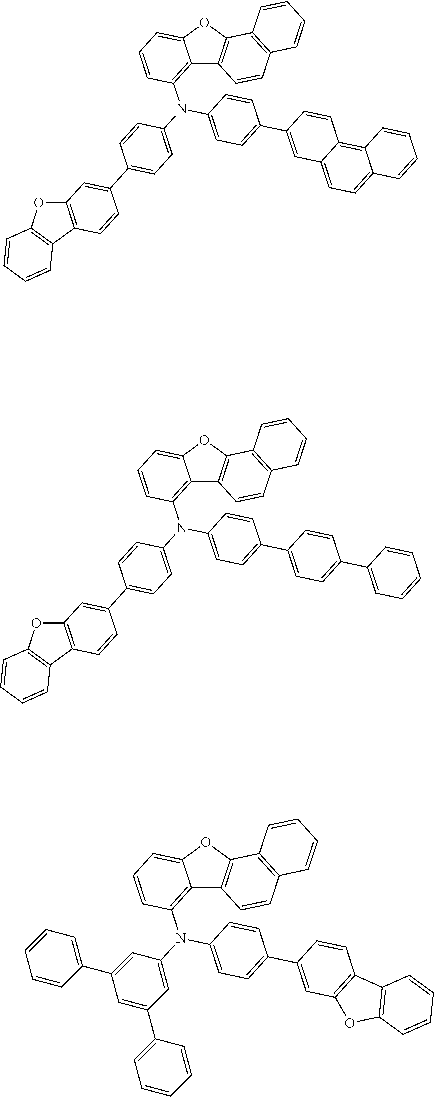 [SMILES: c1ccc2c(c1)ccc1cc(-c3ccc(N(c4ccc(-c5ccc6c(c5)oc5ccccc56)cc4)c4cccc5oc6c7ccccc7ccc6c45)cc3)ccc12 ; c1ccc(-c2ccc(-c3ccc(N(c4ccc(-c5ccc6c(c5)oc5ccccc56)cc4)c4cccc5oc6c7ccccc7ccc6c45)cc3)cc2)cc1 ; c1ccc(-c2cc(-c3ccccc3)cc(N(c3ccc(-c4ccc5c(c4)oc4ccccc45)cc3)c3cccc4oc5c6ccccc6ccc5c34)c2)cc1]

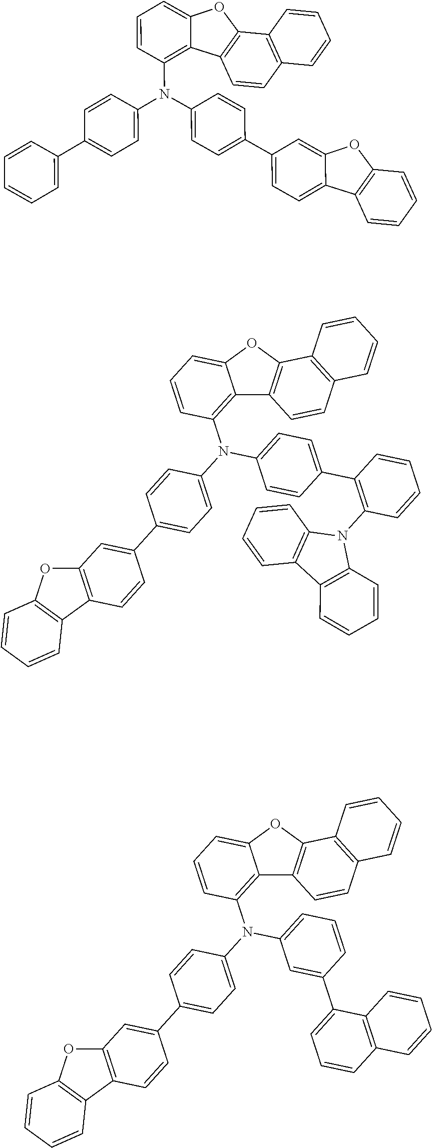 [SMILES: c1ccc(-c2ccc(N(c3ccc(-c4ccc5c(c4)oc4ccccc45)cc3)c3cccc4oc5c6ccccc6ccc5c34)cc2)cc1 ; c1ccc(-n2c3ccccc3c3ccccc32)c(-c2ccc(N(c3ccc(-c4ccc5c(c4)oc4ccccc45)cc3)c3cccc4oc5c6ccccc6ccc5c34)cc2)c1 ; c1cc(-c2cccc3ccccc23)cc(N(c2ccc(-c3ccc4c(c3)oc3ccccc34)cc2)c2cccc3oc4c5ccccc5ccc4c23)c1]

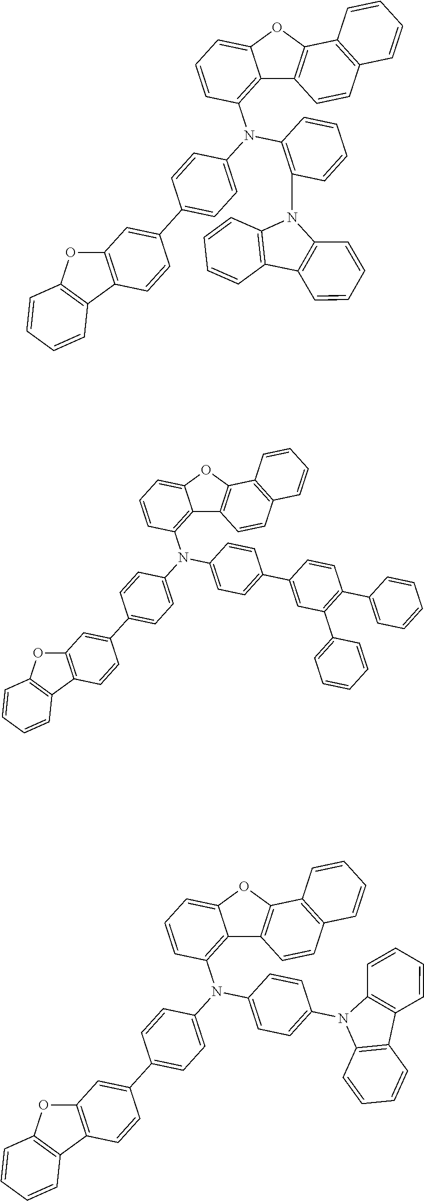 [SMILES: c1ccc(-n2c3ccccc3c3ccccc32)c(N(c2ccc(-c3ccc4c(c3)oc3ccccc34)cc2)c2cccc3oc4c5ccccc5ccc4c23)c1 ; c1ccc(-c2ccc(-c3ccc(N(c4ccc(-c5ccc6c(c5)oc5ccccc56)cc4)c4cccc5oc6c7ccccc7ccc6c45)cc3)cc2-c2ccccc2)cc1 ; c1ccc2c(c1)ccc1c2oc2cccc(N(c3ccc(-c4ccc5c(c4)oc4ccccc45)cc3)c3ccc(-n4c5ccccc5c5ccccc54)cc3)c21]

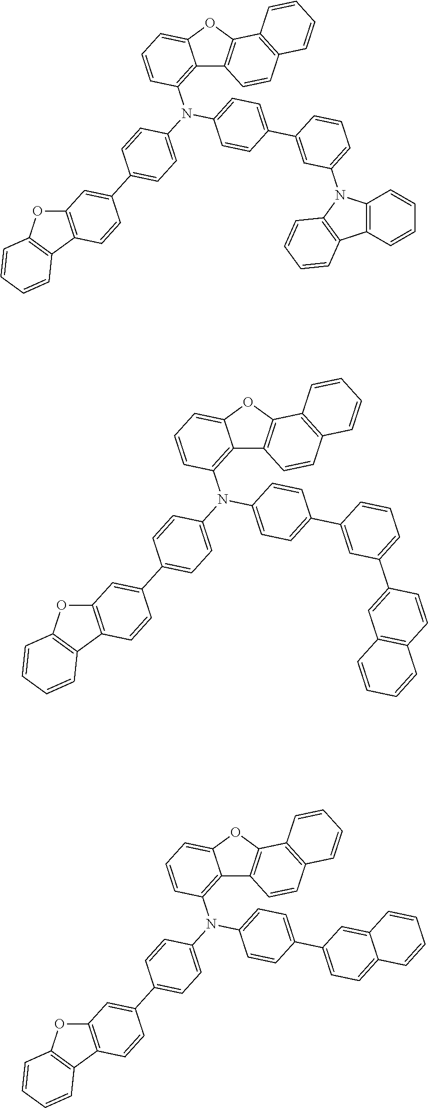 [SMILES: c1cc(-c2ccc(N(c3ccc(-c4ccc5c(c4)oc4ccccc45)cc3)c3cccc4oc5c6ccccc6ccc5c34)cc2)cc(-n2c3ccccc3c3ccccc32)c1 ; c1cc(-c2ccc(N(c3ccc(-c4ccc5c(c4)oc4ccccc45)cc3)c3cccc4oc5c6ccccc6ccc5c34)cc2)cc(-c2ccc3ccccc3c2)c1 ; c1ccc2cc(-c3ccc(N(c4ccc(-c5ccc6c(c5)oc5ccccc56)cc4)c4cccc5oc6c7ccccc7ccc6c45)cc3)ccc2c1]

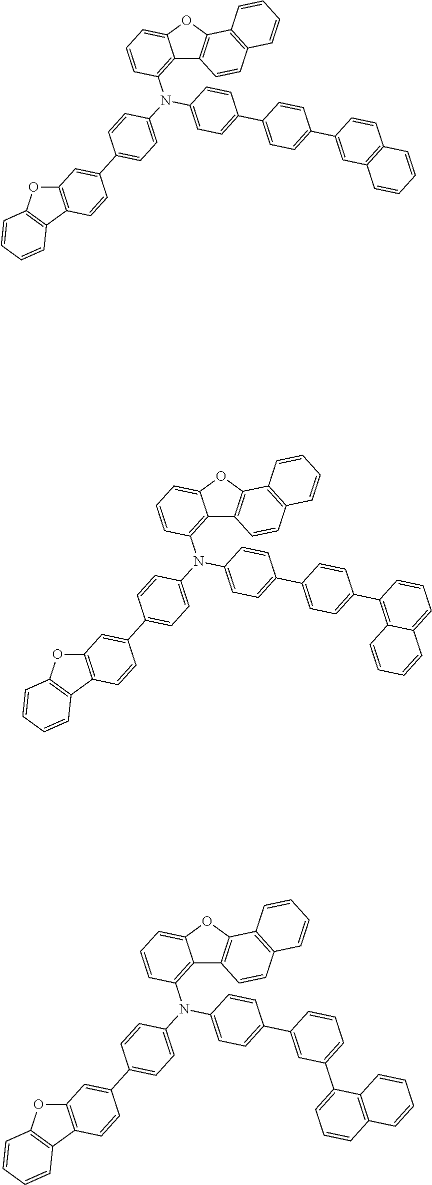 [SMILES: c1ccc2cc(-c3ccc(-c4ccc(N(c5ccc(-c6ccc7c(c6)oc6ccccc67)cc5)c5cccc6oc7c8ccccc8ccc7c56)cc4)cc3)ccc2c1 ; c1ccc2c(-c3ccc(-c4ccc(N(c5ccc(-c6ccc7c(c6)oc6ccccc67)cc5)c5cccc6oc7c8ccccc8ccc7c56)cc4)cc3)cccc2c1 ; c1cc(-c2ccc(N(c3ccc(-c4ccc5c(c4)oc4ccccc45)cc3)c3cccc4oc5c6ccccc6ccc5c34)cc2)cc(-c2cccc3ccccc23)c1]

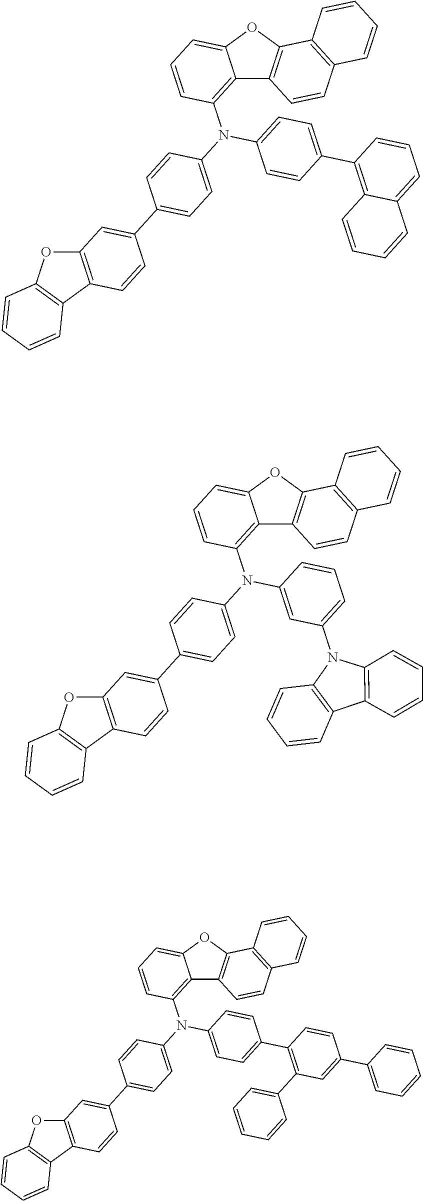 [SMILES: c1ccc2c(-c3ccc(N(c4ccc(-c5ccc6c(c5)oc5ccccc56)cc4)c4cccc5oc6c7ccccc7ccc6c45)cc3)cccc2c1 ; c1cc(N(c2ccc(-c3ccc4c(c3)oc3ccccc34)cc2)c2cccc3oc4c5ccccc5ccc4c23)cc(-n2c3ccccc3c3ccccc32)c1 ; c1ccc(-c2ccc(-c3ccc(N(c4ccc(-c5ccc6c(c5)oc5ccccc56)cc4)c4cccc5oc6c7ccccc7ccc6c45)cc3)c(-c3ccccc3)c2)cc1]

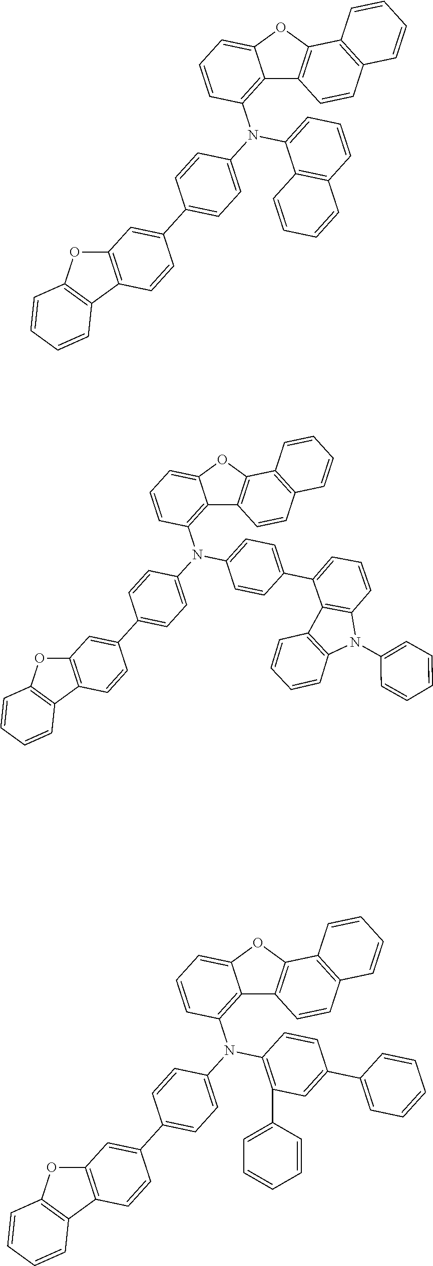 [SMILES: c1ccc2c(N(c3ccc(-c4ccc5c(c4)oc4ccccc45)cc3)c3cccc4oc5c6ccccc6ccc5c34)cccc2c1 ; c1ccc(-n2c3ccccc3c3c(-c4ccc(N(c5ccc(-c6ccc7c(c6)oc6ccccc67)cc5)c5cccc6oc7c8ccccc8ccc7c56)cc4)cccc32)cc1 ; c1ccc(-c2ccc(N(c3ccc(-c4ccc5c(c4)oc4ccccc45)cc3)c3cccc4oc5c6ccccc6ccc5c34)c(-c3ccccc3)c2)cc1]

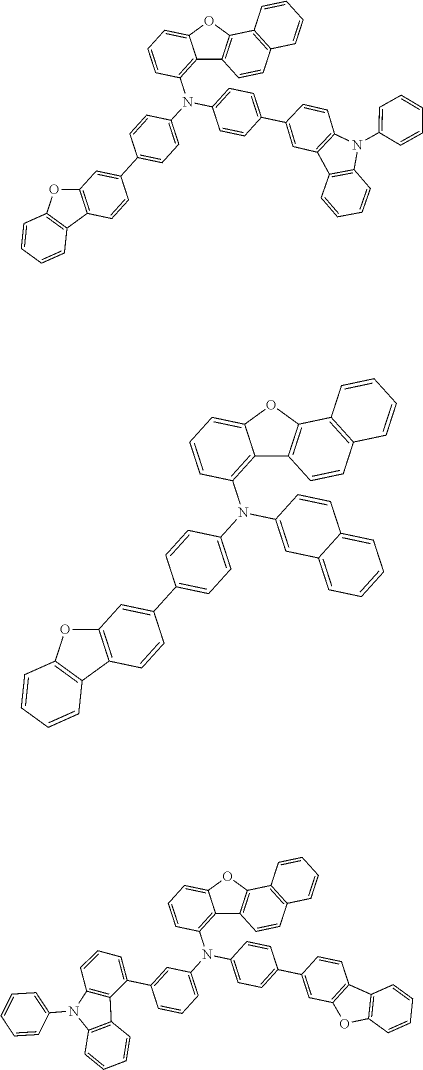 [SMILES: c1ccc(-n2c3ccccc3c3cc(-c4ccc(N(c5ccc(-c6ccc7c(c6)oc6ccccc67)cc5)c5cccc6oc7c8ccccc8ccc7c56)cc4)ccc32)cc1 ; c1ccc2cc(N(c3ccc(-c4ccc5c(c4)oc4ccccc45)cc3)c3cccc4oc5c6ccccc6ccc5c34)ccc2c1 ; c1ccc(-n2c3ccccc3c3c(-c4cccc(N(c5ccc(-c6ccc7c(c6)oc6ccccc67)cc5)c5cccc6oc7c8ccccc8ccc7c56)c4)cccc32)cc1]

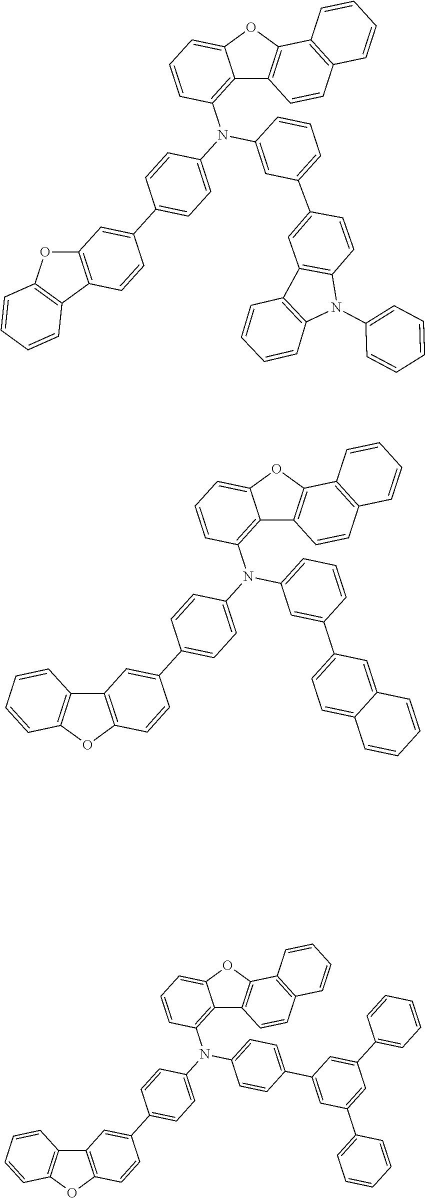 [SMILES: c1ccc(-n2c3ccccc3c3cc(-c4cccc(N(c5ccc(-c6ccc7c(c6)oc6ccccc67)cc5)c5cccc6oc7c8ccccc8ccc7c56)c4)ccc32)cc1 ; c1cc(-c2ccc3ccccc3c2)cc(N(c2ccc(-c3ccc4oc5ccccc5c4c3)cc2)c2cccc3oc4c5ccccc5ccc4c23)c1 ; c1ccc(-c2cc(-c3ccccc3)cc(-c3ccc(N(c4ccc(-c5ccc6oc7ccccc7c6c5)cc4)c4cccc5oc6c7ccccc7ccc6c45)cc3)c2)cc1]

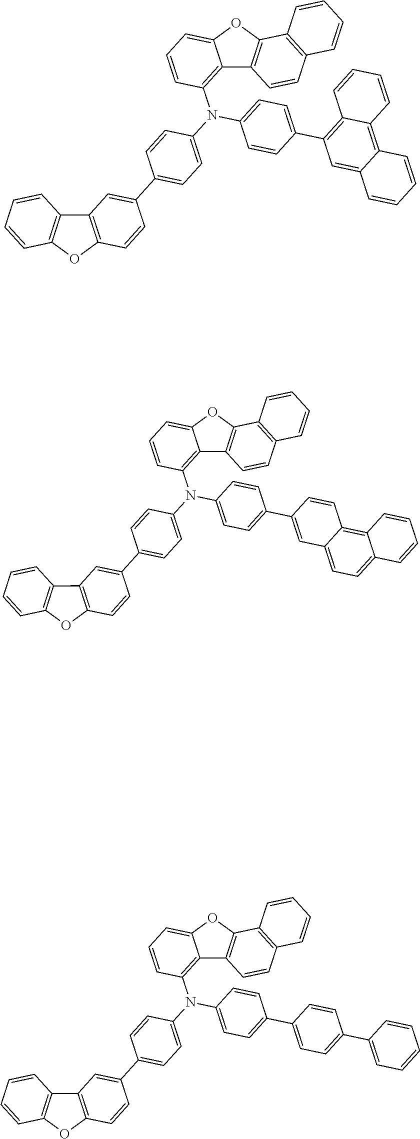 [SMILES: c1ccc2c(c1)cc(-c1ccc(N(c3ccc(-c4ccc5oc6ccccc6c5c4)cc3)c3cccc4oc5c6ccccc6ccc5c34)cc1)c1ccccc12 ; c1ccc2c(c1)ccc1cc(-c3ccc(N(c4ccc(-c5ccc6oc7ccccc7c6c5)cc4)c4cccc5oc6c7ccccc7ccc6c45)cc3)ccc12 ; c1ccc(-c2ccc(-c3ccc(N(c4ccc(-c5ccc6oc7ccccc7c6c5)cc4)c4cccc5oc6c7ccccc7ccc6c45)cc3)cc2)cc1]

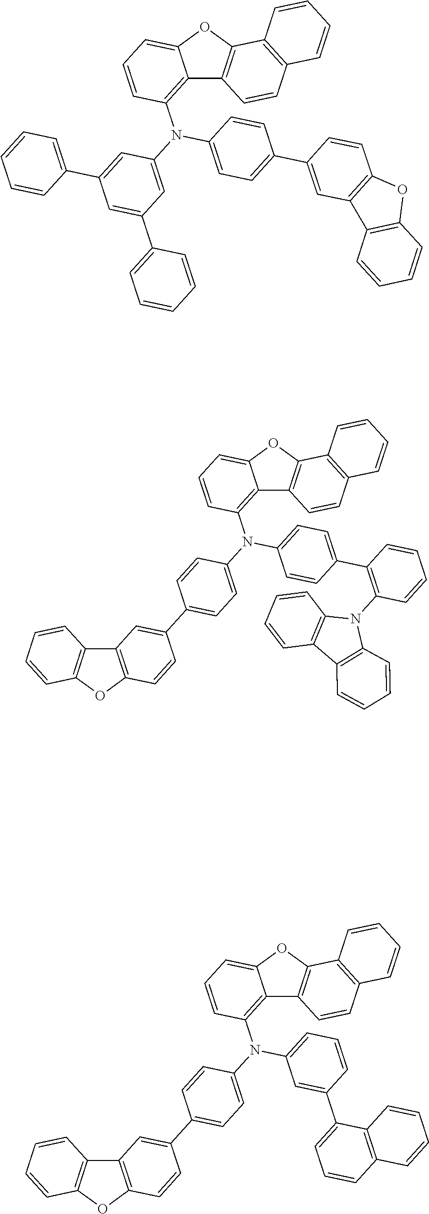 [SMILES: c1ccc(-c2cc(-c3ccccc3)cc(N(c3ccc(-c4ccc5oc6ccccc6c5c4)cc3)c3cccc4oc5c6ccccc6ccc5c34)c2)cc1 ; c1ccc(-n2c3ccccc3c3ccccc32)c(-c2ccc(N(c3ccc(-c4ccc5oc6ccccc6c5c4)cc3)c3cccc4oc5c6ccccc6ccc5c34)cc2)c1 ; c1cc(-c2cccc3ccccc23)cc(N(c2ccc(-c3ccc4oc5ccccc5c4c3)cc2)c2cccc3oc4c5ccccc5ccc4c23)c1]

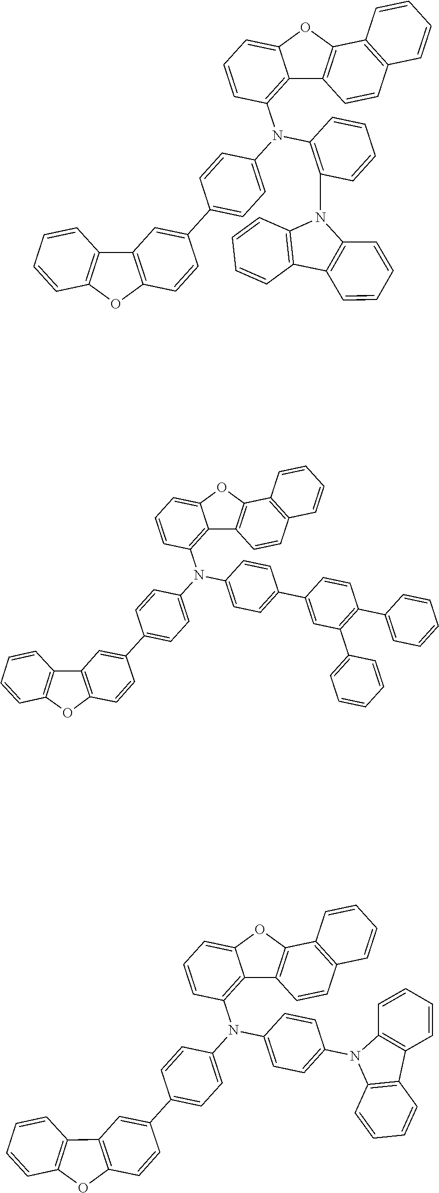 [SMILES: c1ccc(-n2c3ccccc3c3ccccc32)c(N(c2ccc(-c3ccc4oc5ccccc5c4c3)cc2)c2cccc3oc4c5ccccc5ccc4c23)c1 ; c1ccc(-c2ccc(-c3ccc(N(c4ccc(-c5ccc6oc7ccccc7c6c5)cc4)c4cccc5oc6c7ccccc7ccc6c45)cc3)cc2-c2ccccc2)cc1 ; c1ccc2c(c1)ccc1c2oc2cccc(N(c3ccc(-c4ccc5oc6ccccc6c5c4)cc3)c3ccc(-n4c5ccccc5c5ccccc54)cc3)c21]

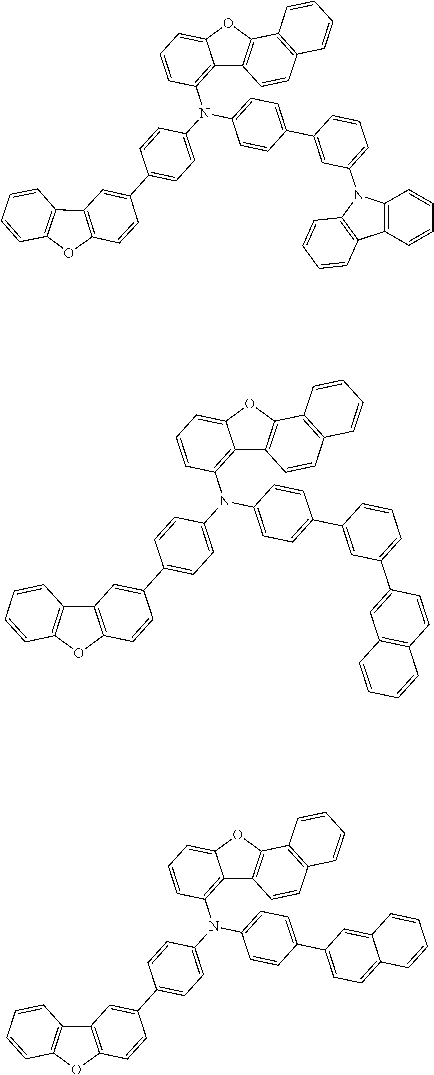 [SMILES: c1cc(-c2ccc(N(c3ccc(-c4ccc5oc6ccccc6c5c4)cc3)c3cccc4oc5c6ccccc6ccc5c34)cc2)cc(-n2c3ccccc3c3ccccc32)c1 ; c1cc(-c2ccc(N(c3ccc(-c4ccc5oc6ccccc6c5c4)cc3)c3cccc4oc5c6ccccc6ccc5c34)cc2)cc(-c2ccc3ccccc3c2)c1 ; c1ccc2cc(-c3ccc(N(c4ccc(-c5ccc6oc7ccccc7c6c5)cc4)c4cccc5oc6c7ccccc7ccc6c45)cc3)ccc2c1]

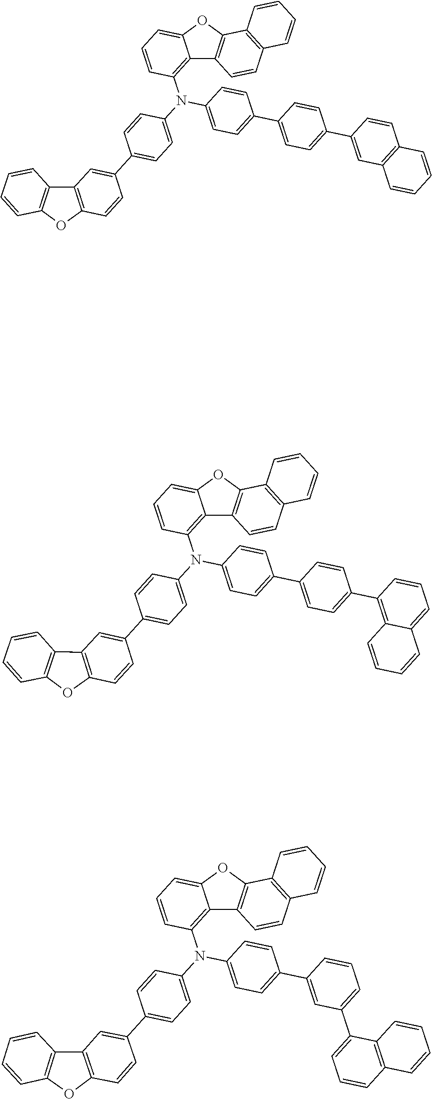 [SMILES: c1ccc2cc(-c3ccc(-c4ccc(N(c5ccc(-c6ccc7oc8ccccc8c7c6)cc5)c5cccc6oc7c8ccccc8ccc7c56)cc4)cc3)ccc2c1 ; c1ccc2c(-c3ccc(-c4ccc(N(c5ccc(-c6ccc7oc8ccccc8c7c6)cc5)c5cccc6oc7c8ccccc8ccc7c56)cc4)cc3)cccc2c1 ; c1cc(-c2ccc(N(c3ccc(-c4ccc5oc6ccccc6c5c4)cc3)c3cccc4oc5c6ccccc6ccc5c34)cc2)cc(-c2cccc3ccccc23)c1]

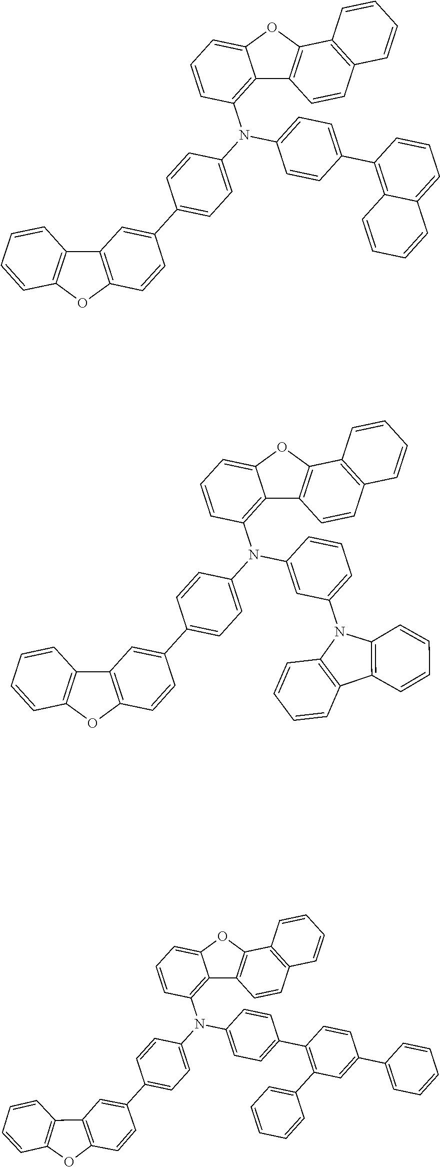 [SMILES: c1ccc2c(-c3ccc(N(c4ccc(-c5ccc6oc7ccccc7c6c5)cc4)c4cccc5oc6c7ccccc7ccc6c45)cc3)cccc2c1 ; c1cc(N(c2ccc(-c3ccc4oc5ccccc5c4c3)cc2)c2cccc3oc4c5ccccc5ccc4c23)cc(-n2c3ccccc3c3ccccc32)c1 ; c1ccc(-c2ccc(-c3ccc(N(c4ccc(-c5ccc6oc7ccccc7c6c5)cc4)c4cccc5oc6c7ccccc7ccc6c45)cc3)c(-c3ccccc3)c2)cc1]

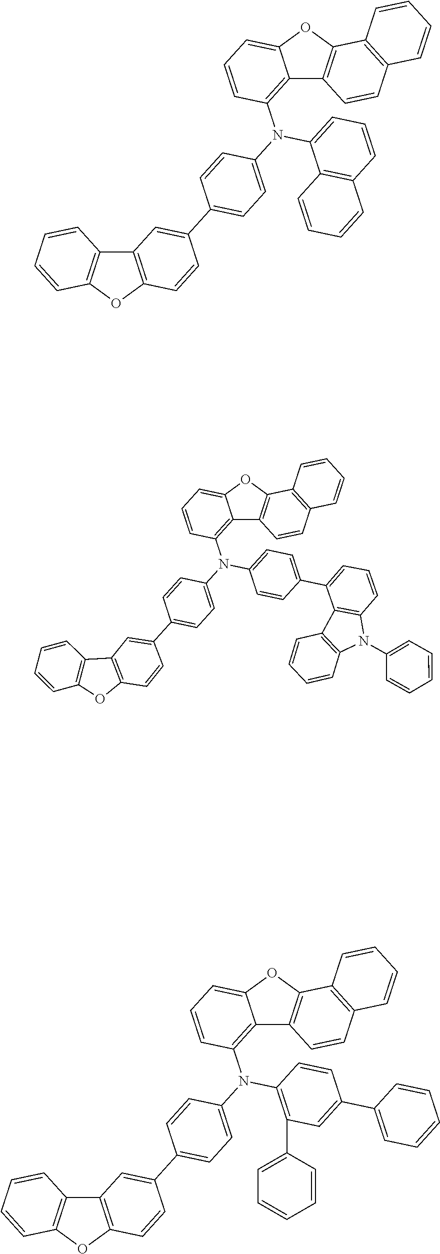 [SMILES: c1ccc2c(N(c3ccc(-c4ccc5oc6ccccc6c5c4)cc3)c3cccc4oc5c6ccccc6ccc5c34)cccc2c1 ; c1ccc(-n2c3ccccc3c3c(-c4ccc(N(c5ccc(-c6ccc7oc8ccccc8c7c6)cc5)c5cccc6oc7c8ccccc8ccc7c56)cc4)cccc32)cc1 ; c1ccc(-c2ccc(N(c3ccc(-c4ccc5oc6ccccc6c5c4)cc3)c3cccc4oc5c6ccccc6ccc5c34)c(-c3ccccc3)c2)cc1]

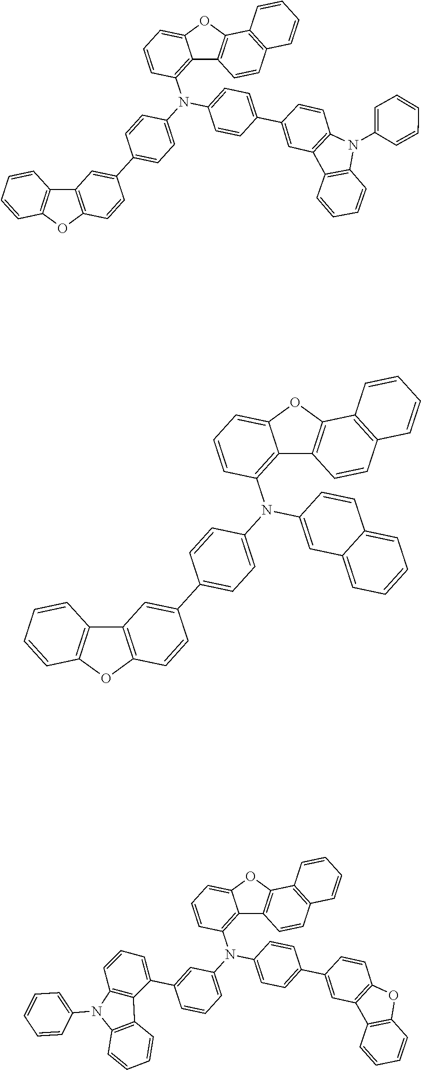 [SMILES: c1ccc(-n2c3ccccc3c3cc(-c4ccc(N(c5ccc(-c6ccc7oc8ccccc8c7c6)cc5)c5cccc6oc7c8ccccc8ccc7c56)cc4)ccc32)cc1 ; c1ccc2cc(N(c3ccc(-c4ccc5oc6ccccc6c5c4)cc3)c3cccc4oc5c6ccccc6ccc5c34)ccc2c1 ; c1ccc(-n2c3ccccc3c3c(-c4cccc(N(c5ccc(-c6ccc7oc8ccccc8c7c6)cc5)c5cccc6oc7c8ccccc8ccc7c56)c4)cccc32)cc1]

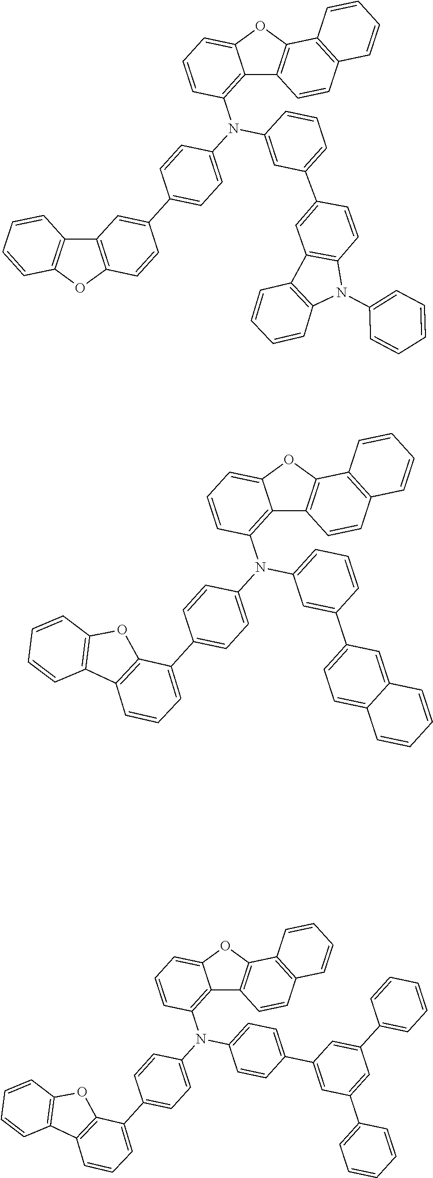 [SMILES: c1ccc(-n2c3ccccc3c3cc(-c4cccc(N(c5ccc(-c6ccc7oc8ccccc8c7c6)cc5)c5cccc6oc7c8ccccc8ccc7c56)c4)ccc32)cc1 ; c1cc(-c2ccc3ccccc3c2)cc(N(c2ccc(-c3cccc4c3oc3ccccc34)cc2)c2cccc3oc4c5ccccc5ccc4c23)c1 ; c1ccc(-c2cc(-c3ccccc3)cc(-c3ccc(N(c4ccc(-c5cccc6c5oc5ccccc56)cc4)c4cccc5oc6c7ccccc7ccc6c45)cc3)c2)cc1]

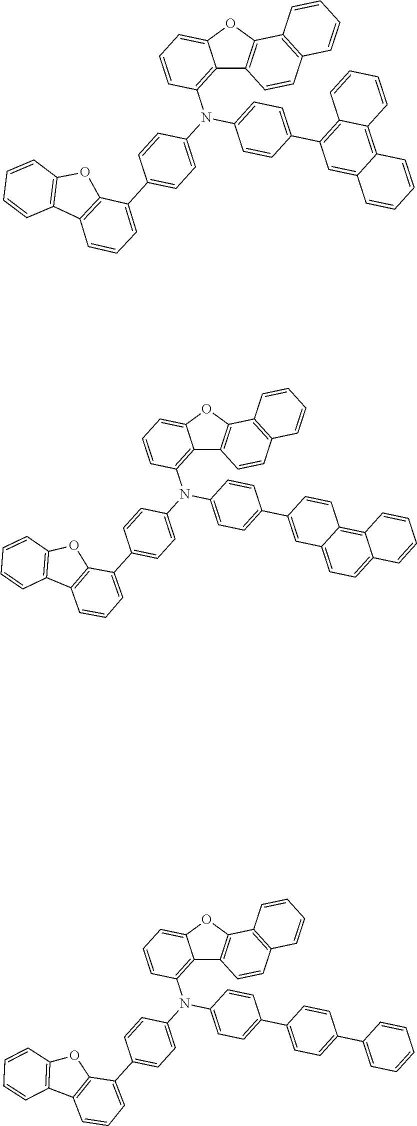 [SMILES: c1ccc2c(c1)cc(-c1ccc(N(c3ccc(-c4cccc5c4oc4ccccc45)cc3)c3cccc4oc5c6ccccc6ccc5c34)cc1)c1ccccc12 ; c1ccc2c(c1)ccc1cc(-c3ccc(N(c4ccc(-c5cccc6c5oc5ccccc56)cc4)c4cccc5oc6c7ccccc7ccc6c45)cc3)ccc12 ; c1ccc(-c2ccc(-c3ccc(N(c4ccc(-c5cccc6c5oc5ccccc56)cc4)c4cccc5oc6c7ccccc7ccc6c45)cc3)cc2)cc1]

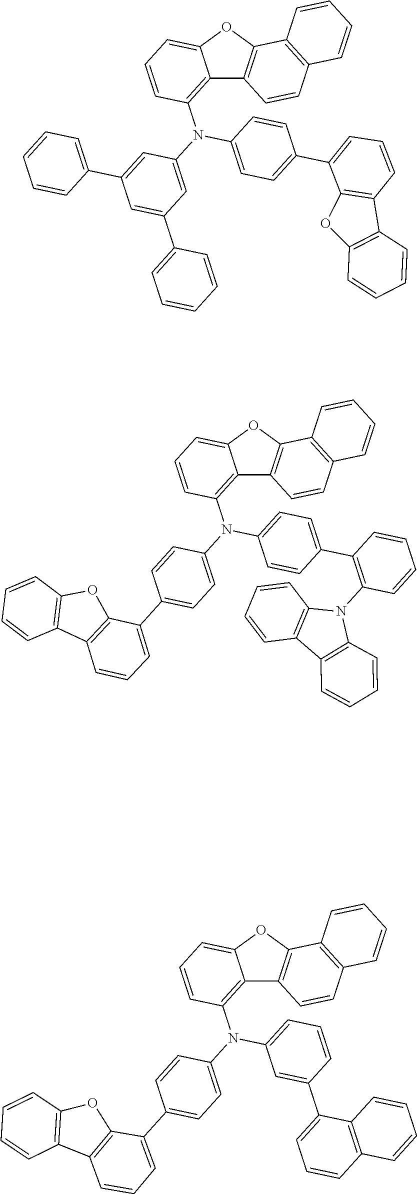 [SMILES: c1ccc(-c2cc(-c3ccccc3)cc(N(c3ccc(-c4cccc5c4oc4ccccc45)cc3)c3cccc4oc5c6ccccc6ccc5c34)c2)cc1 ; c1ccc(-n2c3ccccc3c3ccccc32)c(-c2ccc(N(c3ccc(-c4cccc5c4oc4ccccc45)cc3)c3cccc4oc5c6ccccc6ccc5c34)cc2)c1 ; c1cc(-c2cccc3ccccc23)cc(N(c2ccc(-c3cccc4c3oc3ccccc34)cc2)c2cccc3oc4c5ccccc5ccc4c23)c1]

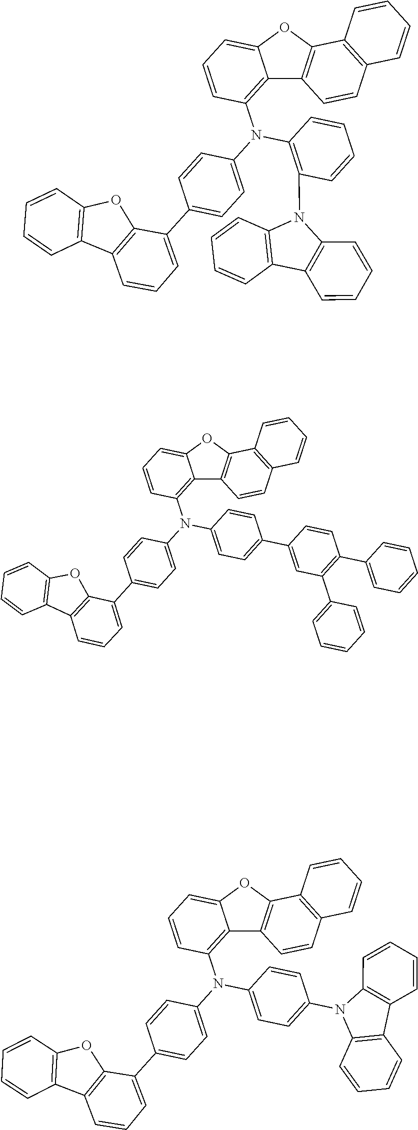 [SMILES: c1ccc(-n2c3ccccc3c3ccccc32)c(N(c2ccc(-c3cccc4c3oc3ccccc34)cc2)c2cccc3oc4c5ccccc5ccc4c23)c1 ; c1ccc(-c2ccc(-c3ccc(N(c4ccc(-c5cccc6c5oc5ccccc56)cc4)c4cccc5oc6c7ccccc7ccc6c45)cc3)cc2-c2ccccc2)cc1 ; c1ccc2c(c1)ccc1c2oc2cccc(N(c3ccc(-c4cccc5c4oc4ccccc45)cc3)c3ccc(-n4c5ccccc5c5ccccc54)cc3)c21]

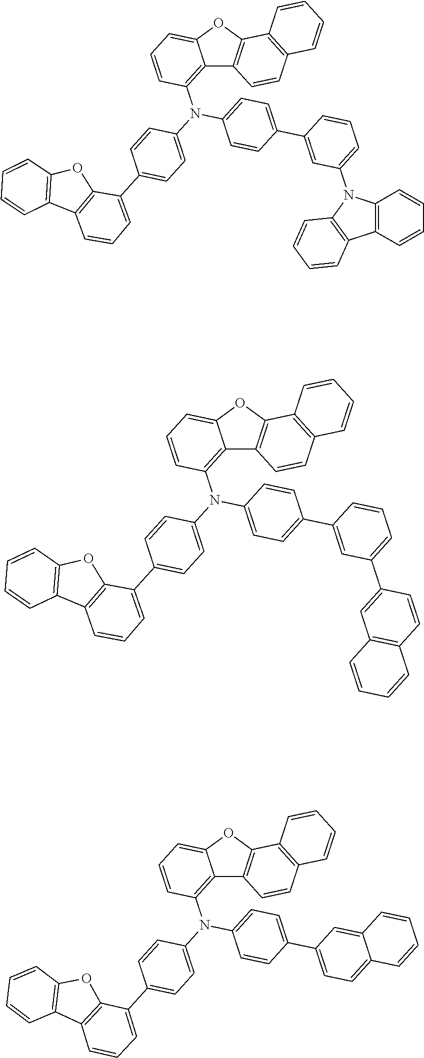 [SMILES: c1cc(-c2ccc(N(c3ccc(-c4cccc5c4oc4ccccc45)cc3)c3cccc4oc5c6ccccc6ccc5c34)cc2)cc(-n2c3ccccc3c3ccccc32)c1 ; c1cc(-c2ccc(N(c3ccc(-c4cccc5c4oc4ccccc45)cc3)c3cccc4oc5c6ccccc6ccc5c34)cc2)cc(-c2ccc3ccccc3c2)c1 ; c1ccc2cc(-c3ccc(N(c4ccc(-c5cccc6c5oc5ccccc56)cc4)c4cccc5oc6c7ccccc7ccc6c45)cc3)ccc2c1]

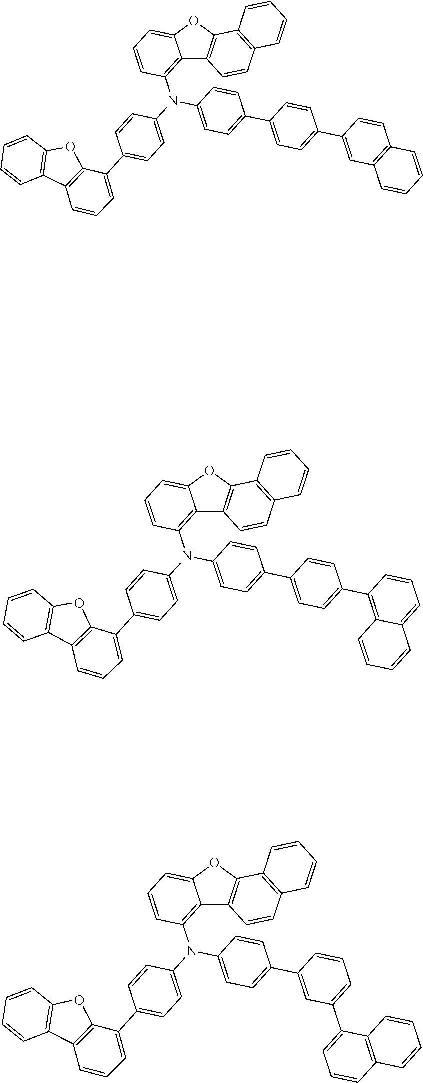 [SMILES: c1ccc2cc(-c3ccc(-c4ccc(N(c5ccc(-c6cccc7c6oc6ccccc67)cc5)c5cccc6oc7c8ccccc8ccc7c56)cc4)cc3)ccc2c1 ; c1ccc2c(-c3ccc(-c4ccc(N(c5ccc(-c6cccc7c6oc6ccccc67)cc5)c5cccc6oc7c8ccccc8ccc7c56)cc4)cc3)cccc2c1 ; c1cc(-c2ccc(N(c3ccc(-c4cccc5c4oc4ccccc45)cc3)c3cccc4oc5c6ccccc6ccc5c34)cc2)cc(-c2cccc3ccccc23)c1]

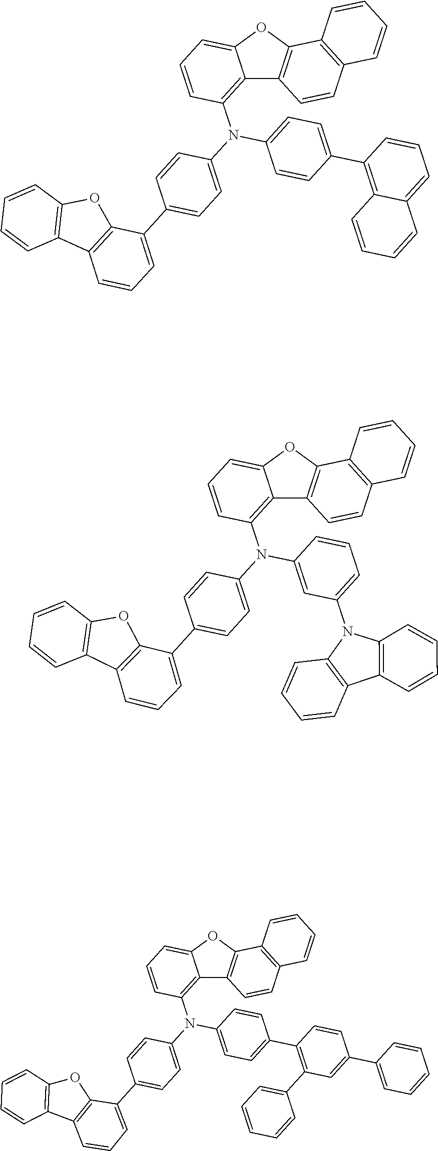 [SMILES: c1ccc2c(-c3ccc(N(c4ccc(-c5cccc6c5oc5ccccc56)cc4)c4cccc5oc6c7ccccc7ccc6c45)cc3)cccc2c1 ; c1cc(N(c2ccc(-c3cccc4c3oc3ccccc34)cc2)c2cccc3oc4c5ccccc5ccc4c23)cc(-n2c3ccccc3c3ccccc32)c1 ; c1ccc(-c2ccc(-c3ccc(N(c4ccc(-c5cccc6c5oc5ccccc56)cc4)c4cccc5oc6c7ccccc7ccc6c45)cc3)c(-c3ccccc3)c2)cc1]

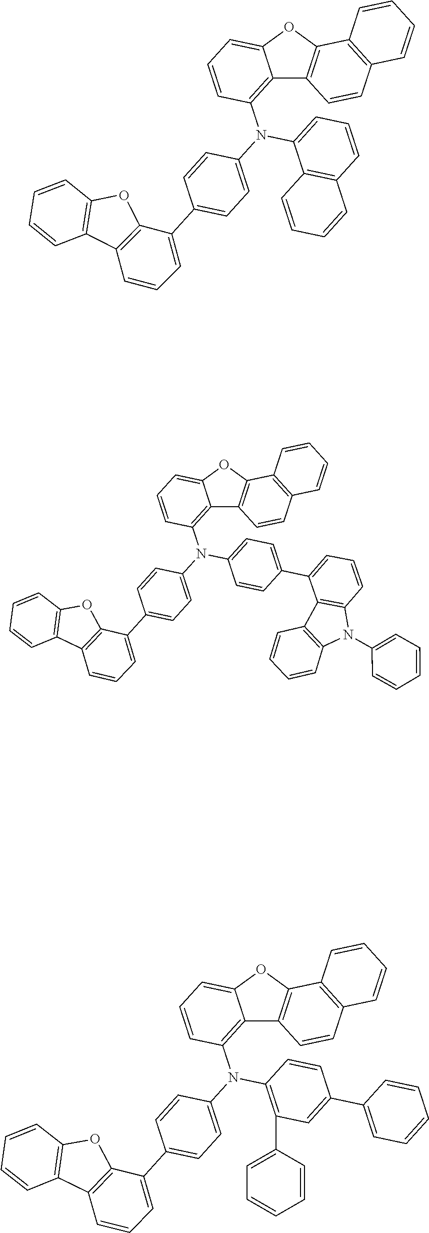 [SMILES: c1ccc2c(N(c3ccc(-c4cccc5c4oc4ccccc45)cc3)c3cccc4oc5c6ccccc6ccc5c34)cccc2c1 ; c1ccc(-n2c3ccccc3c3c(-c4ccc(N(c5ccc(-c6cccc7c6oc6ccccc67)cc5)c5cccc6oc7c8ccccc8ccc7c56)cc4)cccc32)cc1 ; c1ccc(-c2ccc(N(c3ccc(-c4cccc5c4oc4ccccc45)cc3)c3cccc4oc5c6ccccc6ccc5c34)c(-c3ccccc3)c2)cc1]

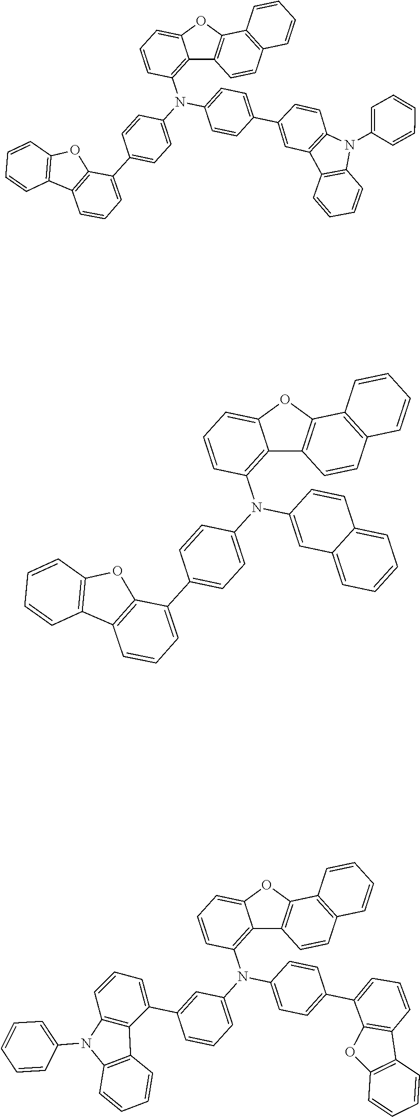 [SMILES: c1ccc(-n2c3ccccc3c3cc(-c4ccc(N(c5ccc(-c6cccc7c6oc6ccccc67)cc5)c5cccc6oc7c8ccccc8ccc7c56)cc4)ccc32)cc1 ; c1ccc2cc(N(c3ccc(-c4cccc5c4oc4ccccc45)cc3)c3cccc4oc5c6ccccc6ccc5c34)ccc2c1 ; c1ccc(-n2c3ccccc3c3c(-c4cccc(N(c5ccc(-c6cccc7c6oc6ccccc67)cc5)c5cccc6oc7c8ccccc8ccc7c56)c4)cccc32)cc1]

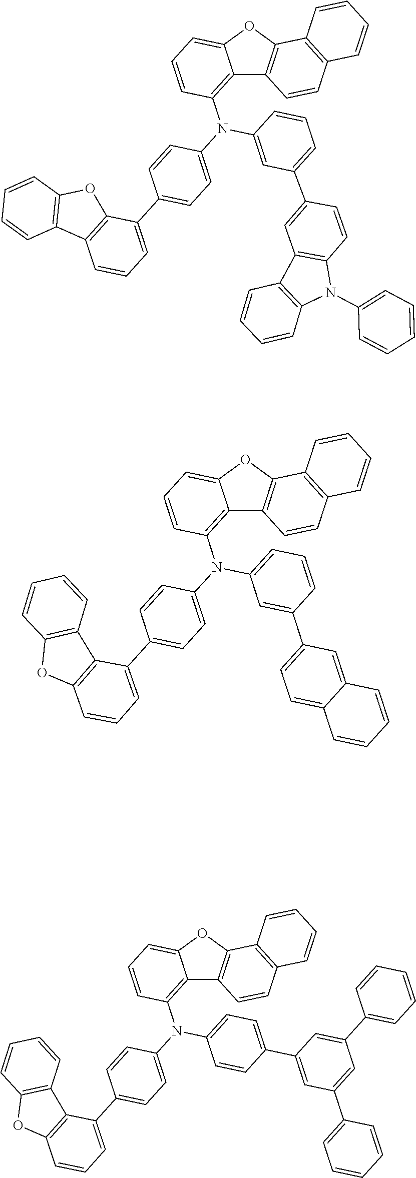 [SMILES: c1ccc(-n2c3ccccc3c3cc(-c4cccc(N(c5ccc(-c6cccc7c6oc6ccccc67)cc5)c5cccc6oc7c8ccccc8ccc7c56)c4)ccc32)cc1 ; c1cc(-c2ccc3ccccc3c2)cc(N(c2ccc(-c3cccc4oc5ccccc5c34)cc2)c2cccc3oc4c5ccccc5ccc4c23)c1 ; c1ccc(-c2cc(-c3ccccc3)cc(-c3ccc(N(c4ccc(-c5cccc6oc7ccccc7c56)cc4)c4cccc5oc6c7ccccc7ccc6c45)cc3)c2)cc1]

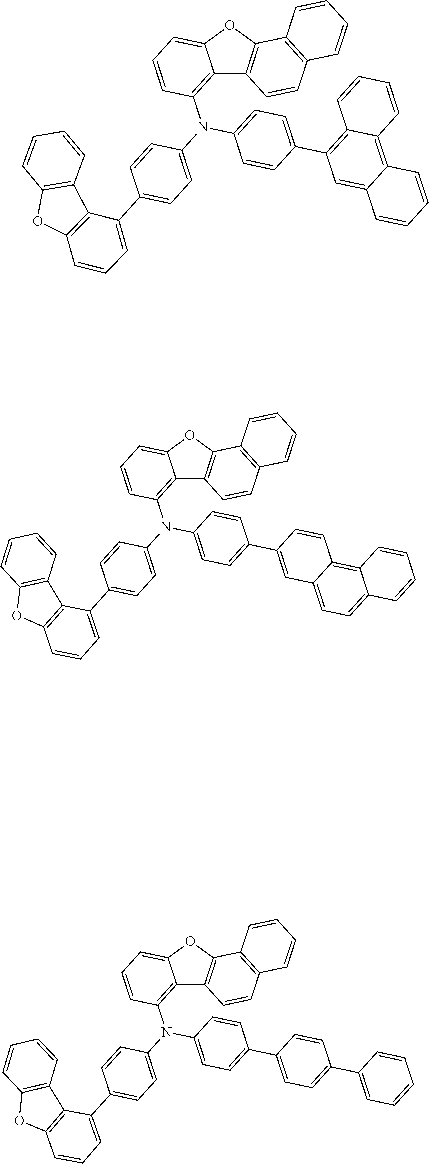 [SMILES: c1ccc2c(c1)cc(-c1ccc(N(c3ccc(-c4cccc5oc6ccccc6c45)cc3)c3cccc4oc5c6ccccc6ccc5c34)cc1)c1ccccc12 ; c1ccc2c(c1)ccc1cc(-c3ccc(N(c4ccc(-c5cccc6oc7ccccc7c56)cc4)c4cccc5oc6c7ccccc7ccc6c45)cc3)ccc12 ; c1ccc(-c2ccc(-c3ccc(N(c4ccc(-c5cccc6oc7ccccc7c56)cc4)c4cccc5oc6c7ccccc7ccc6c45)cc3)cc2)cc1]

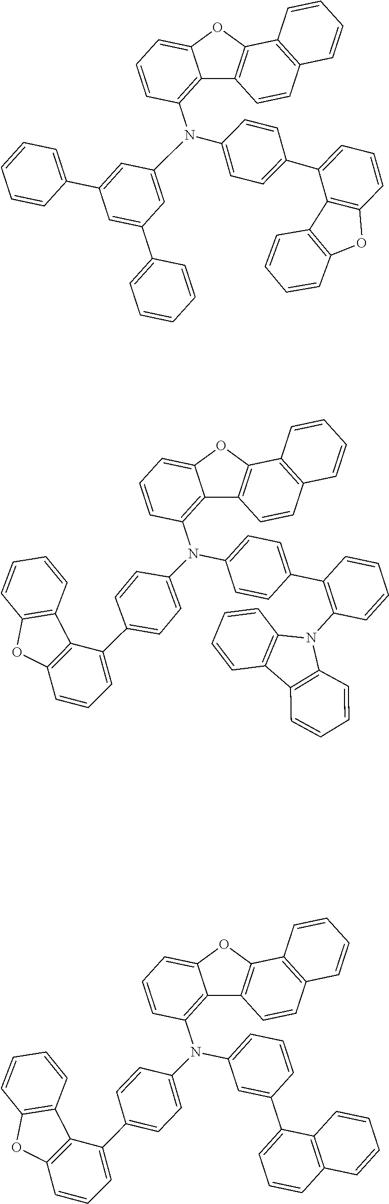 [SMILES: c1ccc(-c2cc(-c3ccccc3)cc(N(c3ccc(-c4cccc5oc6ccccc6c45)cc3)c3cccc4oc5c6ccccc6ccc5c34)c2)cc1 ; c1ccc(-n2c3ccccc3c3ccccc32)c(-c2ccc(N(c3ccc(-c4cccc5oc6ccccc6c45)cc3)c3cccc4oc5c6ccccc6ccc5c34)cc2)c1 ; c1cc(-c2cccc3ccccc23)cc(N(c2ccc(-c3cccc4oc5ccccc5c34)cc2)c2cccc3oc4c5ccccc5ccc4c23)c1]

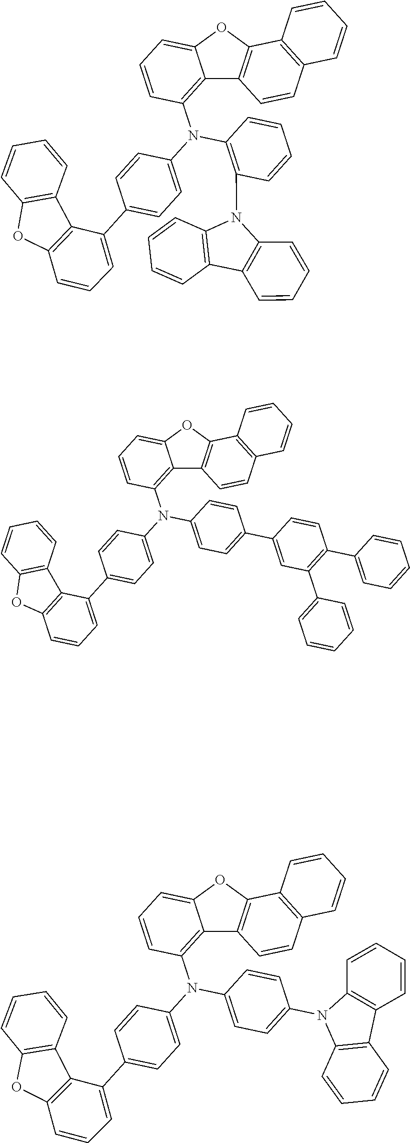 [SMILES: c1ccc(-n2c3ccccc3c3ccccc32)c(N(c2ccc(-c3cccc4oc5ccccc5c34)cc2)c2cccc3oc4c5ccccc5ccc4c23)c1 ; c1ccc(-c2ccc(-c3ccc(N(c4ccc(-c5cccc6oc7ccccc7c56)cc4)c4cccc5oc6c7ccccc7ccc6c45)cc3)cc2-c2ccccc2)cc1 ; c1ccc2c(c1)ccc1c2oc2cccc(N(c3ccc(-c4cccc5oc6ccccc6c45)cc3)c3ccc(-n4c5ccccc5c5ccccc54)cc3)c21]

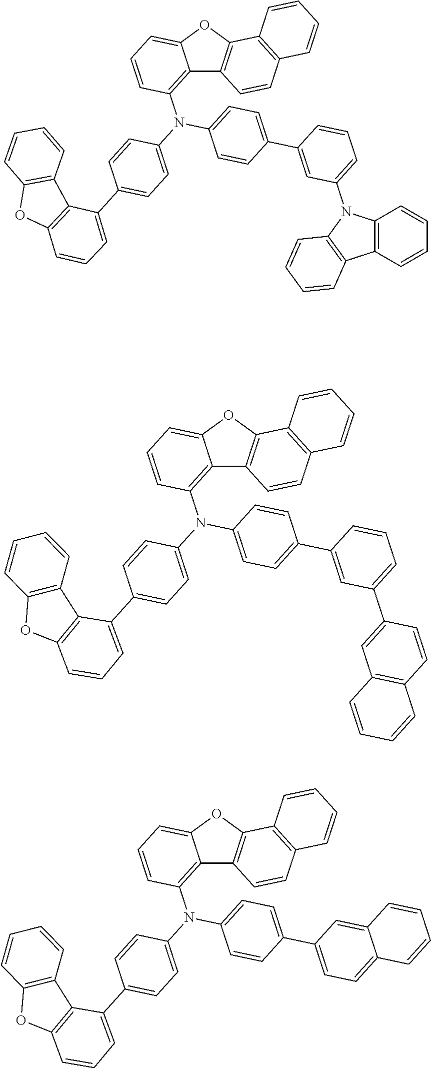 [SMILES: c1cc(-c2ccc(N(c3ccc(-c4cccc5oc6ccccc6c45)cc3)c3cccc4oc5c6ccccc6ccc5c34)cc2)cc(-n2c3ccccc3c3ccccc32)c1 ; c1cc(-c2ccc(N(c3ccc(-c4cccc5oc6ccccc6c45)cc3)c3cccc4oc5c6ccccc6ccc5c34)cc2)cc(-c2ccc3ccccc3c2)c1 ; c1ccc2cc(-c3ccc(N(c4ccc(-c5cccc6oc7ccccc7c56)cc4)c4cccc5oc6c7ccccc7ccc6c45)cc3)ccc2c1]

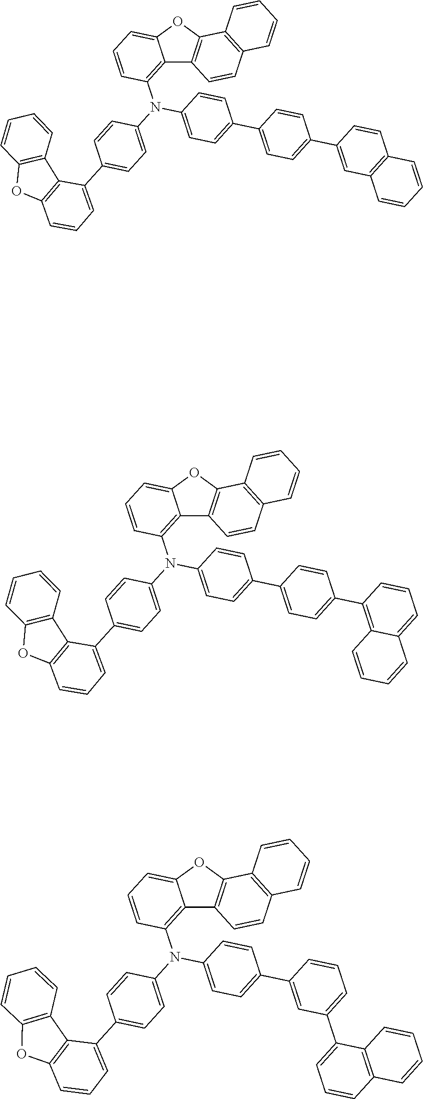 [SMILES: c1ccc2cc(-c3ccc(-c4ccc(N(c5ccc(-c6cccc7oc8ccccc8c67)cc5)c5cccc6oc7c8ccccc8ccc7c56)cc4)cc3)ccc2c1 ; c1ccc2c(-c3ccc(-c4ccc(N(c5ccc(-c6cccc7oc8ccccc8c67)cc5)c5cccc6oc7c8ccccc8ccc7c56)cc4)cc3)cccc2c1 ; c1cc(-c2ccc(N(c3ccc(-c4cccc5oc6ccccc6c45)cc3)c3cccc4oc5c6ccccc6ccc5c34)cc2)cc(-c2cccc3ccccc23)c1]

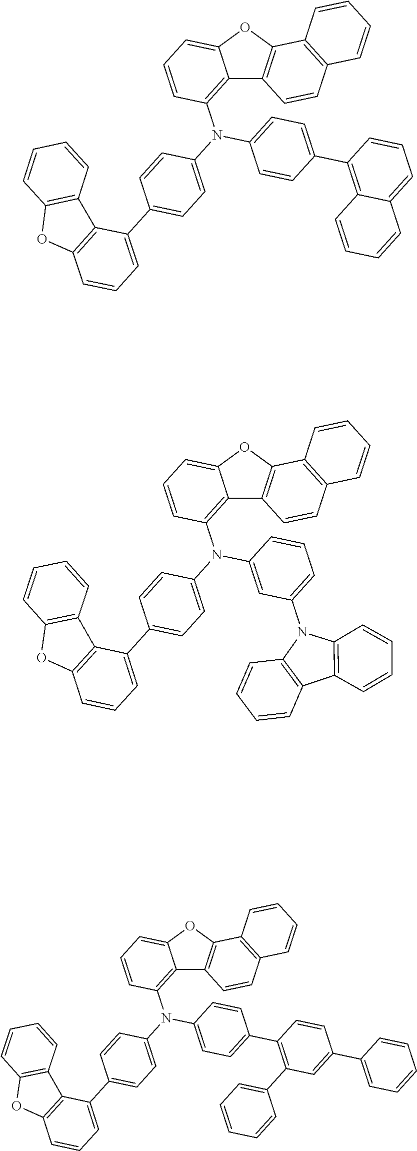 [SMILES: c1ccc2c(-c3ccc(N(c4ccc(-c5cccc6oc7ccccc7c56)cc4)c4cccc5oc6c7ccccc7ccc6c45)cc3)cccc2c1 ; c1cc(N(c2ccc(-c3cccc4oc5ccccc5c34)cc2)c2cccc3oc4c5ccccc5ccc4c23)cc(-n2c3ccccc3c3ccccc32)c1 ; c1ccc(-c2ccc(-c3ccc(N(c4ccc(-c5cccc6oc7ccccc7c56)cc4)c4cccc5oc6c7ccccc7ccc6c45)cc3)c(-c3ccccc3)c2)cc1]

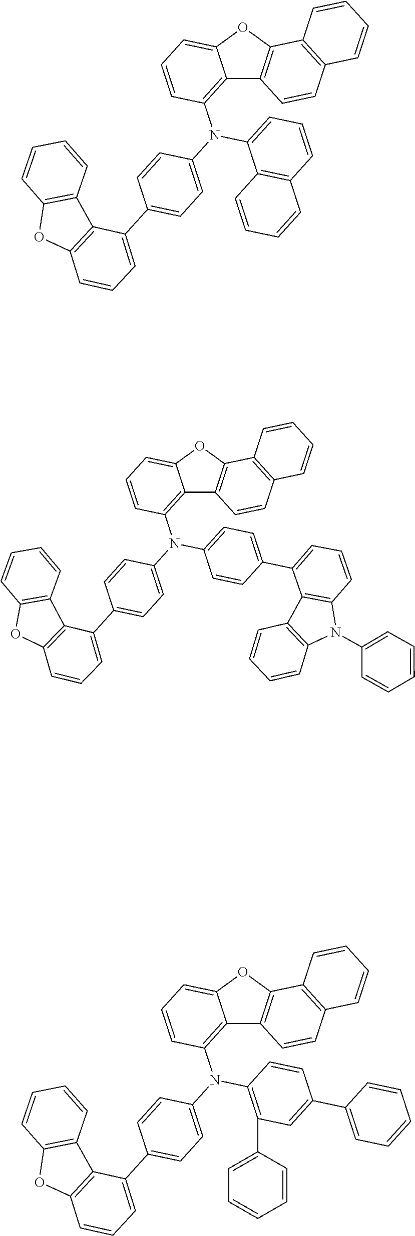 [SMILES: c1ccc2c(N(c3ccc(-c4cccc5oc6ccccc6c45)cc3)c3cccc4oc5c6ccccc6ccc5c34)cccc2c1 ; c1ccc(-n2c3ccccc3c3c(-c4ccc(N(c5ccc(-c6cccc7oc8ccccc8c67)cc5)c5cccc6oc7c8ccccc8ccc7c56)cc4)cccc32)cc1 ; c1ccc(-c2ccc(N(c3ccc(-c4cccc5oc6ccccc6c45)cc3)c3cccc4oc5c6ccccc6ccc5c34)c(-c3ccccc3)c2)cc1]

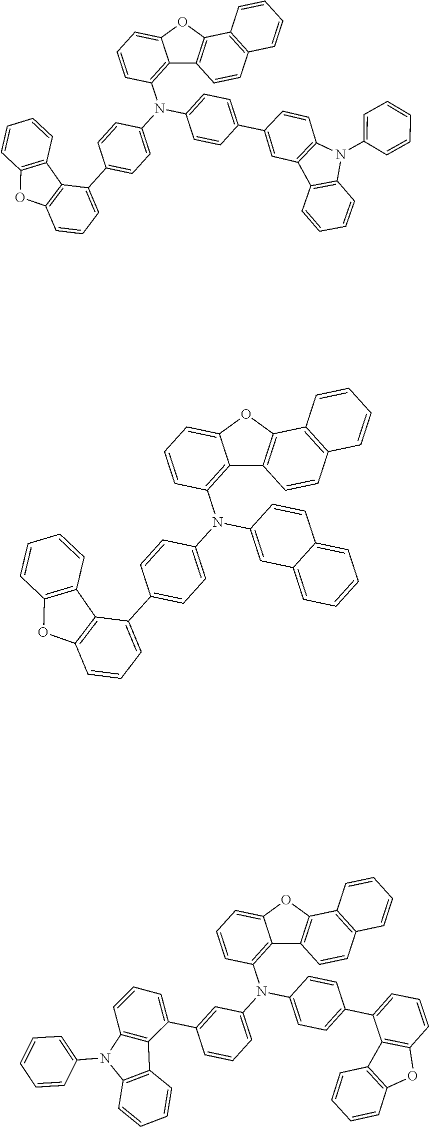 [SMILES: c1ccc(-n2c3ccccc3c3cc(-c4ccc(N(c5ccc(-c6cccc7oc8ccccc8c67)cc5)c5cccc6oc7c8ccccc8ccc7c56)cc4)ccc32)cc1 ; c1ccc2cc(N(c3ccc(-c4cccc5oc6ccccc6c45)cc3)c3cccc4oc5c6ccccc6ccc5c34)ccc2c1 ; c1ccc(-n2c3ccccc3c3c(-c4cccc(N(c5ccc(-c6cccc7oc8ccccc8c67)cc5)c5cccc6oc7c8ccccc8ccc7c56)c4)cccc32)cc1]

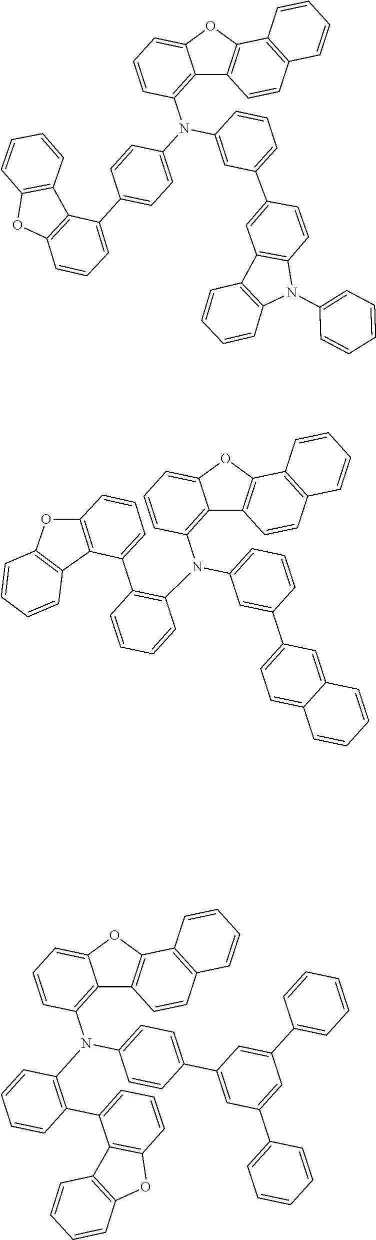 [SMILES: c1ccc(-n2c3ccccc3c3cc(-c4cccc(N(c5ccc(-c6cccc7oc8ccccc8c67)cc5)c5cccc6oc7c8ccccc8ccc7c56)c4)ccc32)cc1 ; c1cc(-c2ccc3ccccc3c2)cc(N(c2ccccc2-c2cccc3oc4ccccc4c23)c2cccc3oc4c5ccccc5ccc4c23)c1 ; c1ccc(-c2cc(-c3ccccc3)cc(-c3ccc(N(c4ccccc4-c4cccc5oc6ccccc6c45)c4cccc5oc6c7ccccc7ccc6c45)cc3)c2)cc1]

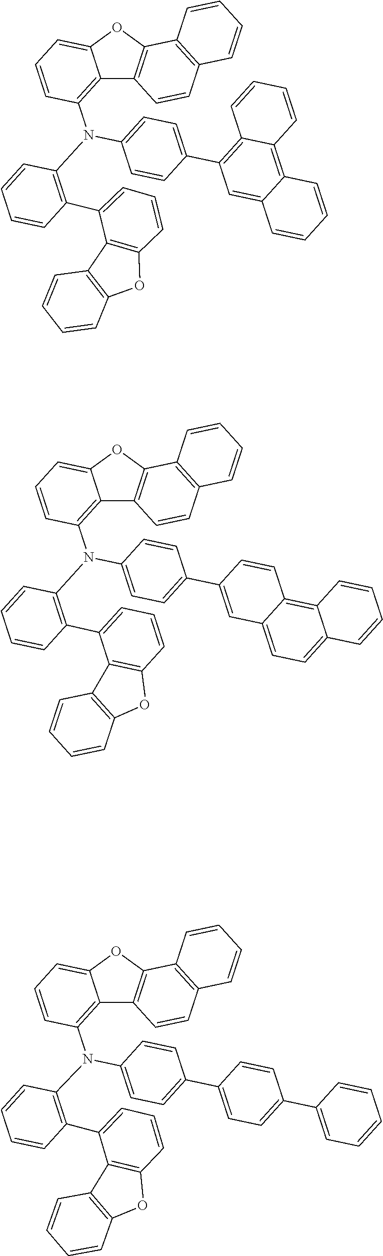 [SMILES: c1ccc(N(c2ccc(-c3cc4ccccc4c4ccccc34)cc2)c2cccc3oc4c5ccccc5ccc4c23)c(-c2cccc3oc4ccccc4c23)c1 ; c1ccc(N(c2ccc(-c3ccc4c(ccc5ccccc54)c3)cc2)c2cccc3oc4c5ccccc5ccc4c23)c(-c2cccc3oc4ccccc4c23)c1 ; c1ccc(-c2ccc(-c3ccc(N(c4ccccc4-c4cccc5oc6ccccc6c45)c4cccc5oc6c7ccccc7ccc6c45)cc3)cc2)cc1]

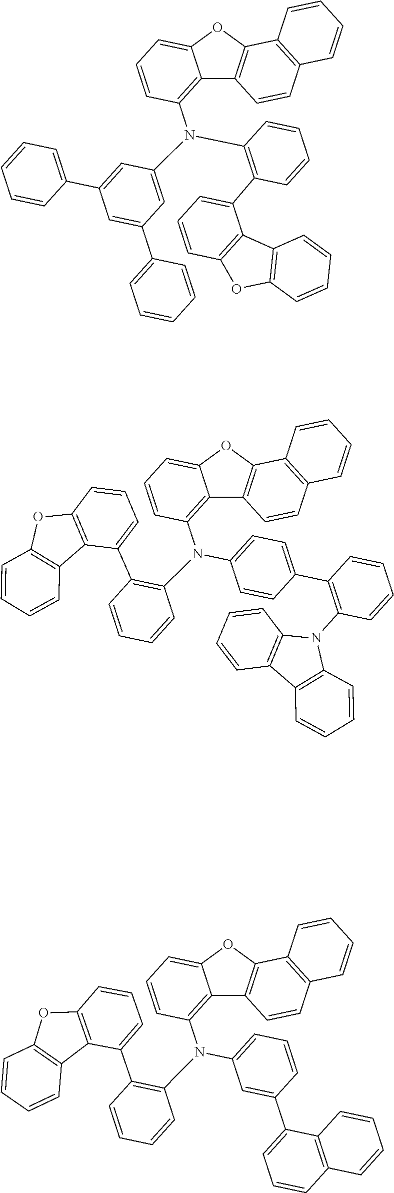 [SMILES: c1ccc(-c2cc(-c3ccccc3)cc(N(c3ccccc3-c3cccc4oc5ccccc5c34)c3cccc4oc5c6ccccc6ccc5c34)c2)cc1 ; c1ccc(N(c2ccc(-c3ccccc3-n3c4ccccc4c4ccccc43)cc2)c2cccc3oc4c5ccccc5ccc4c23)c(-c2cccc3oc4ccccc4c23)c1 ; c1cc(-c2cccc3ccccc23)cc(N(c2ccccc2-c2cccc3oc4ccccc4c23)c2cccc3oc4c5ccccc5ccc4c23)c1]

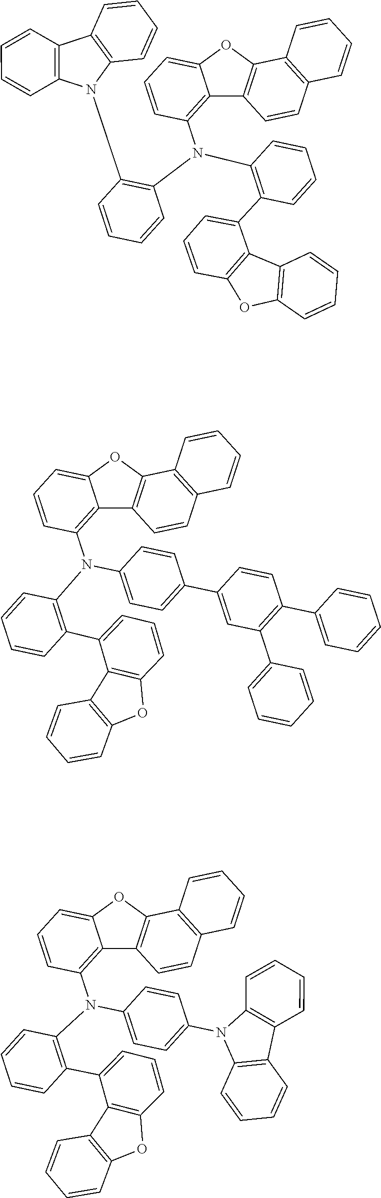 [SMILES: c1ccc(N(c2ccccc2-n2c3ccccc3c3ccccc32)c2cccc3oc4c5ccccc5ccc4c23)c(-c2cccc3oc4ccccc4c23)c1 ; c1ccc(-c2ccc(-c3ccc(N(c4ccccc4-c4cccc5oc6ccccc6c45)c4cccc5oc6c7ccccc7ccc6c45)cc3)cc2-c2ccccc2)cc1 ; c1ccc(N(c2ccc(-n3c4ccccc4c4ccccc43)cc2)c2cccc3oc4c5ccccc5ccc4c23)c(-c2cccc3oc4ccccc4c23)c1]

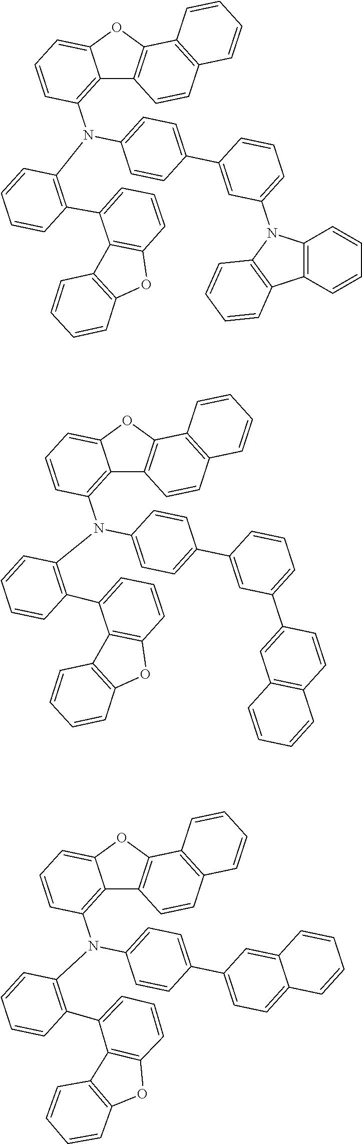 [SMILES: c1cc(-c2ccc(N(c3ccccc3-c3cccc4oc5ccccc5c34)c3cccc4oc5c6ccccc6ccc5c34)cc2)cc(-n2c3ccccc3c3ccccc32)c1 ; c1cc(-c2ccc(N(c3ccccc3-c3cccc4oc5ccccc5c34)c3cccc4oc5c6ccccc6ccc5c34)cc2)cc(-c2ccc3ccccc3c2)c1 ; c1ccc(N(c2ccc(-c3ccc4ccccc4c3)cc2)c2cccc3oc4c5ccccc5ccc4c23)c(-c2cccc3oc4ccccc4c23)c1]

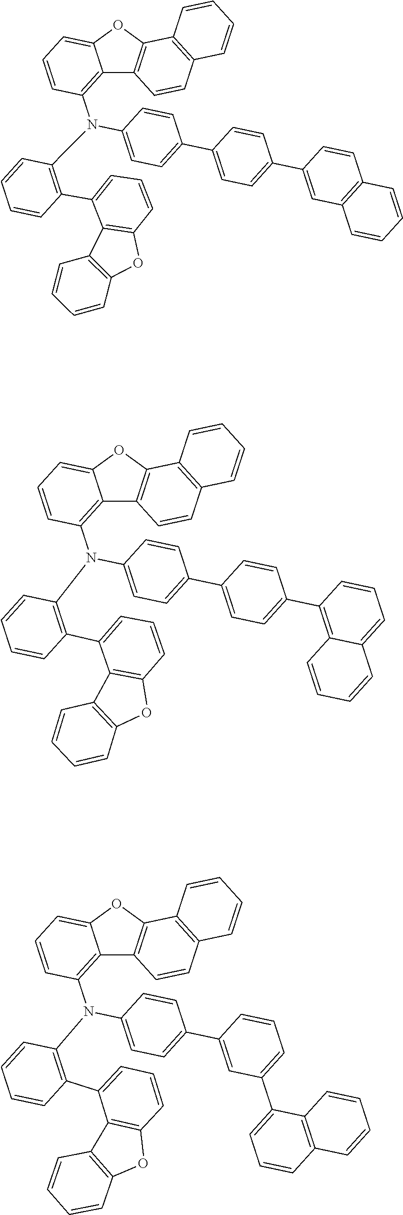 [SMILES: c1ccc(N(c2ccc(-c3ccc(-c4ccc5ccccc5c4)cc3)cc2)c2cccc3oc4c5ccccc5ccc4c23)c(-c2cccc3oc4ccccc4c23)c1 ; c1ccc(N(c2ccc(-c3ccc(-c4cccc5ccccc45)cc3)cc2)c2cccc3oc4c5ccccc5ccc4c23)c(-c2cccc3oc4ccccc4c23)c1 ; c1cc(-c2ccc(N(c3ccccc3-c3cccc4oc5ccccc5c34)c3cccc4oc5c6ccccc6ccc5c34)cc2)cc(-c2cccc3ccccc23)c1]

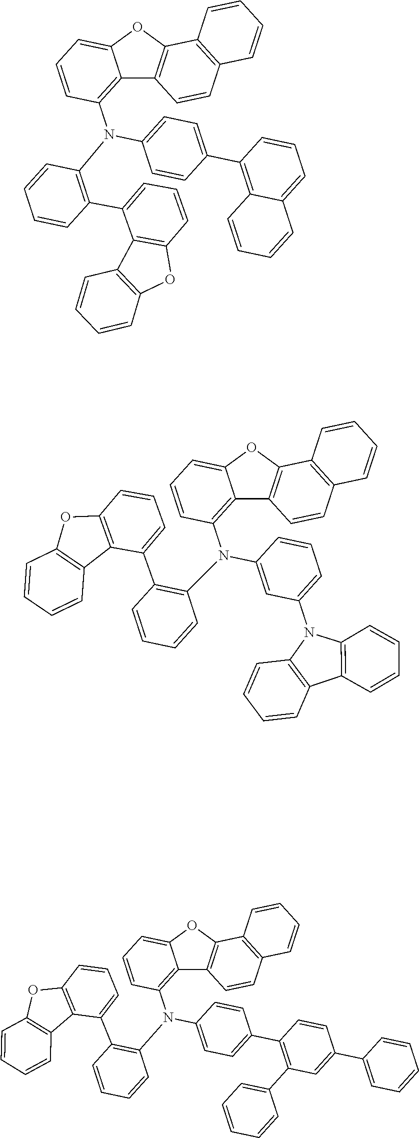 [SMILES: c1ccc(N(c2ccc(-c3cccc4ccccc34)cc2)c2cccc3oc4c5ccccc5ccc4c23)c(-c2cccc3oc4ccccc4c23)c1 ; c1cc(N(c2ccccc2-c2cccc3oc4ccccc4c23)c2cccc3oc4c5ccccc5ccc4c23)cc(-n2c3ccccc3c3ccccc32)c1 ; c1ccc(-c2ccc(-c3ccc(N(c4ccccc4-c4cccc5oc6ccccc6c45)c4cccc5oc6c7ccccc7ccc6c45)cc3)c(-c3ccccc3)c2)cc1]

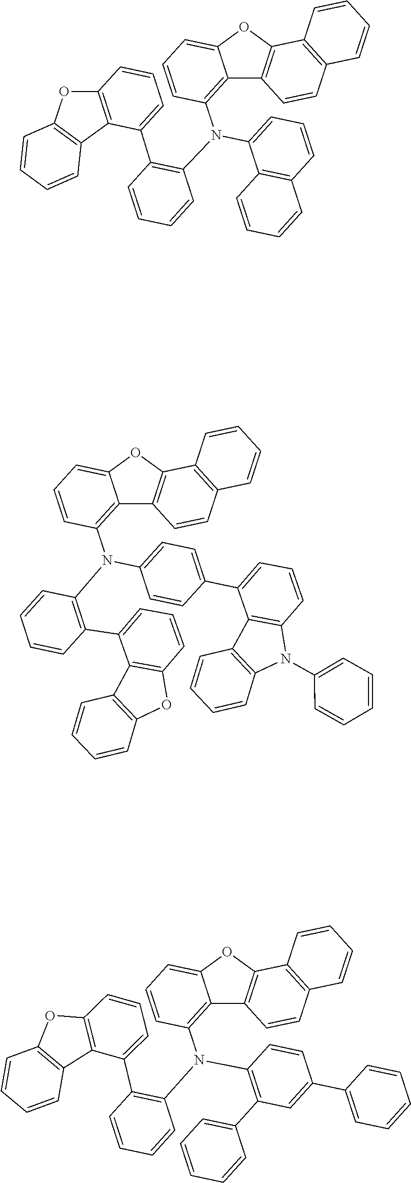 [SMILES: c1ccc(N(c2cccc3ccccc23)c2cccc3oc4c5ccccc5ccc4c23)c(-c2cccc3oc4ccccc4c23)c1 ; c1ccc(-n2c3ccccc3c3c(-c4ccc(N(c5ccccc5-c5cccc6oc7ccccc7c56)c5cccc6oc7c8ccccc8ccc7c56)cc4)cccc32)cc1 ; c1ccc(-c2ccc(N(c3ccccc3-c3cccc4oc5ccccc5c34)c3cccc4oc5c6ccccc6ccc5c34)c(-c3ccccc3)c2)cc1]

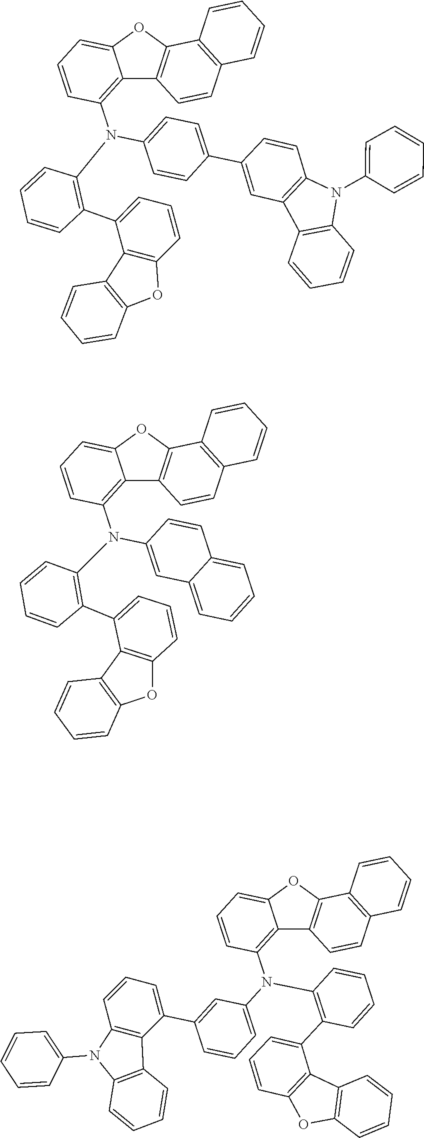 [SMILES: c1ccc(-n2c3ccccc3c3cc(-c4ccc(N(c5ccccc5-c5cccc6oc7ccccc7c56)c5cccc6oc7c8ccccc8ccc7c56)cc4)ccc32)cc1 ; c1ccc(N(c2ccc3ccccc3c2)c2cccc3oc4c5ccccc5ccc4c23)c(-c2cccc3oc4ccccc4c23)c1 ; c1ccc(-n2c3ccccc3c3c(-c4cccc(N(c5ccccc5-c5cccc6oc7ccccc7c56)c5cccc6oc7c8ccccc8ccc7c56)c4)cccc32)cc1]

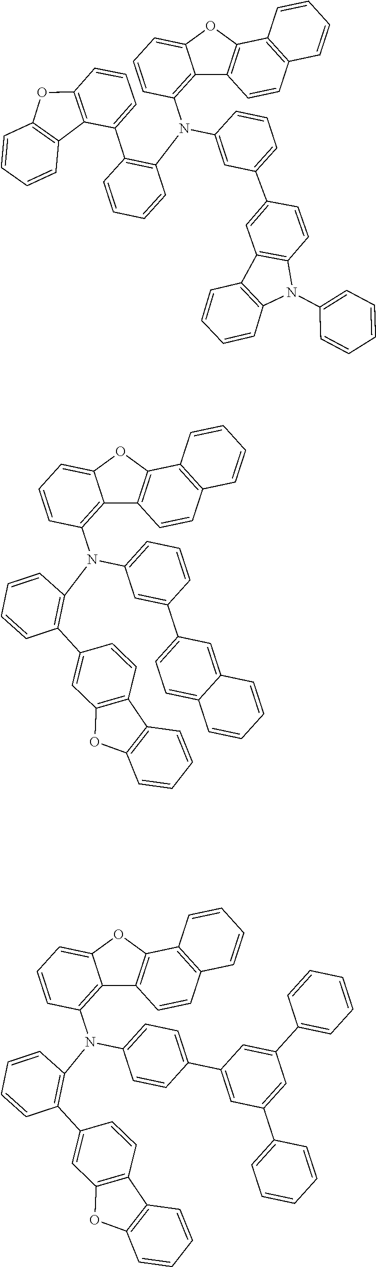 [SMILES: c1ccc(-n2c3ccccc3c3cc(-c4cccc(N(c5ccccc5-c5cccc6oc7ccccc7c56)c5cccc6oc7c8ccccc8ccc7c56)c4)ccc32)cc1 ; c1cc(-c2ccc3ccccc3c2)cc(N(c2ccccc2-c2ccc3c(c2)oc2ccccc23)c2cccc3oc4c5ccccc5ccc4c23)c1 ; c1ccc(-c2cc(-c3ccccc3)cc(-c3ccc(N(c4ccccc4-c4ccc5c(c4)oc4ccccc45)c4cccc5oc6c7ccccc7ccc6c45)cc3)c2)cc1]

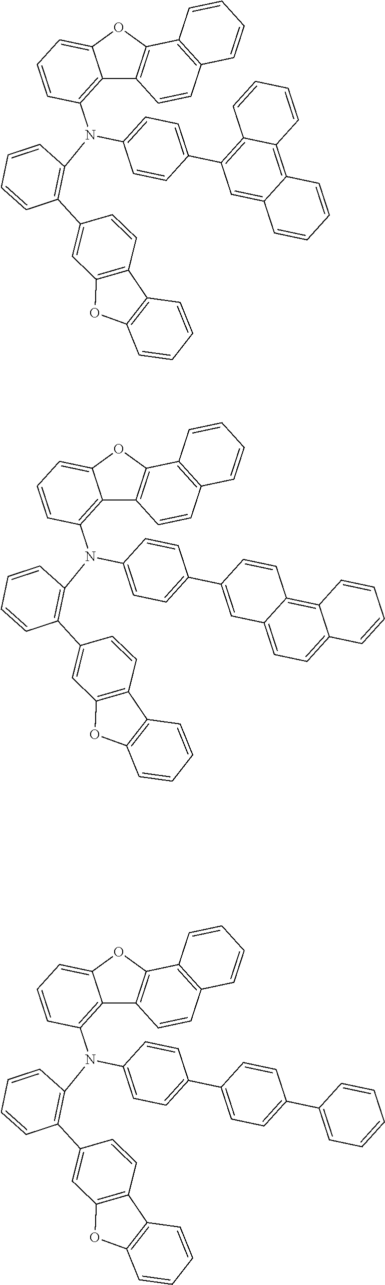 [SMILES: c1ccc(N(c2ccc(-c3cc4ccccc4c4ccccc34)cc2)c2cccc3oc4c5ccccc5ccc4c23)c(-c2ccc3c(c2)oc2ccccc23)c1 ; c1ccc(N(c2ccc(-c3ccc4c(ccc5ccccc54)c3)cc2)c2cccc3oc4c5ccccc5ccc4c23)c(-c2ccc3c(c2)oc2ccccc23)c1 ; c1ccc(-c2ccc(-c3ccc(N(c4ccccc4-c4ccc5c(c4)oc4ccccc45)c4cccc5oc6c7ccccc7ccc6c45)cc3)cc2)cc1]

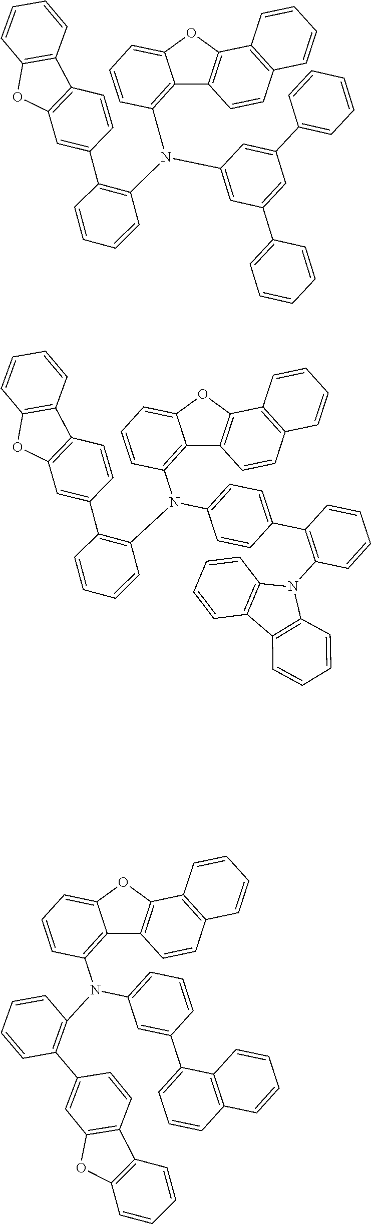 [SMILES: c1ccc(-c2cc(-c3ccccc3)cc(N(c3ccccc3-c3ccc4c(c3)oc3ccccc34)c3cccc4oc5c6ccccc6ccc5c34)c2)cc1 ; c1ccc(N(c2ccc(-c3ccccc3-n3c4ccccc4c4ccccc43)cc2)c2cccc3oc4c5ccccc5ccc4c23)c(-c2ccc3c(c2)oc2ccccc23)c1 ; c1cc(-c2cccc3ccccc23)cc(N(c2ccccc2-c2ccc3c(c2)oc2ccccc23)c2cccc3oc4c5ccccc5ccc4c23)c1]

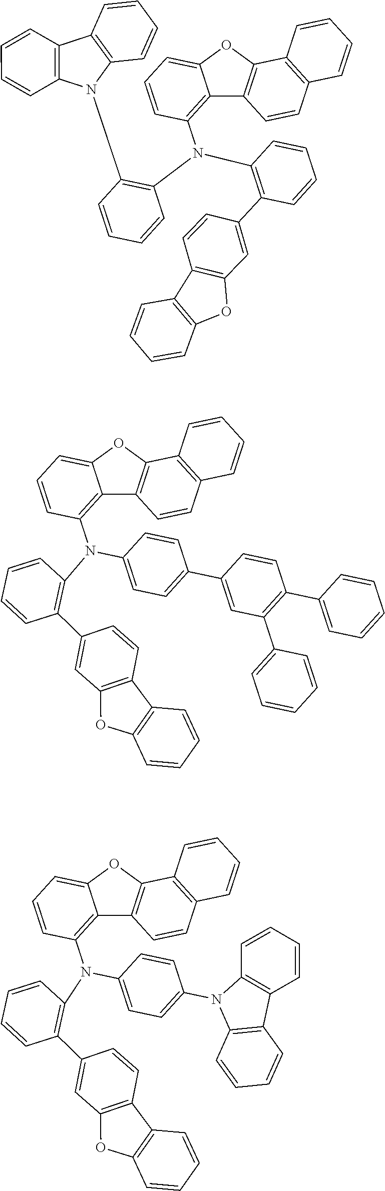 [SMILES: c1ccc(N(c2ccccc2-n2c3ccccc3c3ccccc32)c2cccc3oc4c5ccccc5ccc4c23)c(-c2ccc3c(c2)oc2ccccc23)c1 ; c1ccc(-c2ccc(-c3ccc(N(c4ccccc4-c4ccc5c(c4)oc4ccccc45)c4cccc5oc6c7ccccc7ccc6c45)cc3)cc2-c2ccccc2)cc1 ; c1ccc(N(c2ccc(-n3c4ccccc4c4ccccc43)cc2)c2cccc3oc4c5ccccc5ccc4c23)c(-c2ccc3c(c2)oc2ccccc23)c1]

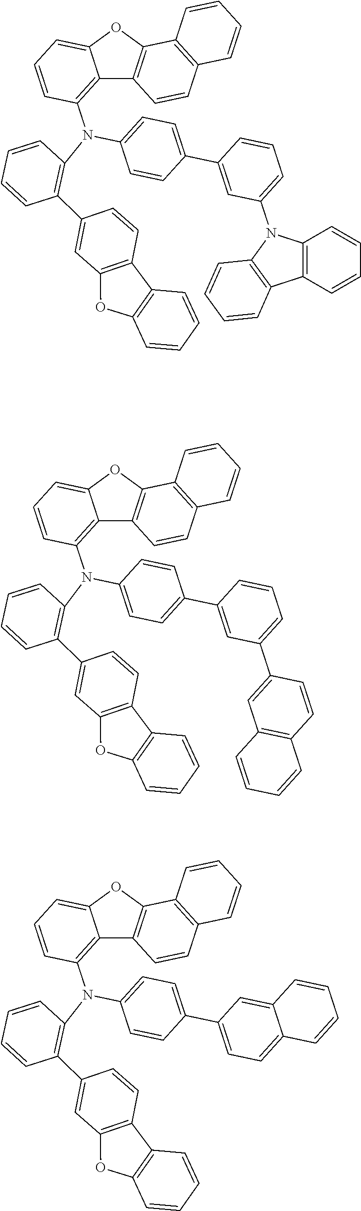 [SMILES: c1cc(-c2ccc(N(c3ccccc3-c3ccc4c(c3)oc3ccccc34)c3cccc4oc5c6ccccc6ccc5c34)cc2)cc(-n2c3ccccc3c3ccccc32)c1 ; c1cc(-c2ccc(N(c3ccccc3-c3ccc4c(c3)oc3ccccc34)c3cccc4oc5c6ccccc6ccc5c34)cc2)cc(-c2ccc3ccccc3c2)c1 ; c1ccc(N(c2ccc(-c3ccc4ccccc4c3)cc2)c2cccc3oc4c5ccccc5ccc4c23)c(-c2ccc3c(c2)oc2ccccc23)c1]

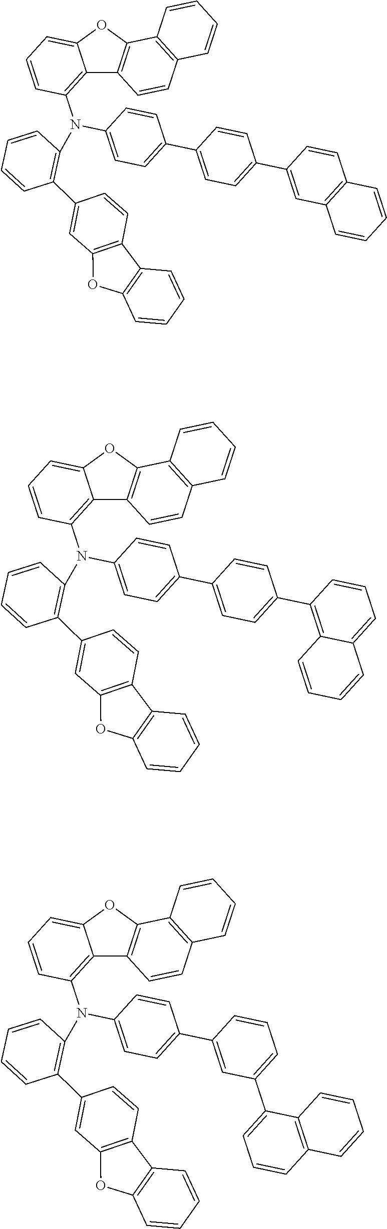 [SMILES: c1ccc(N(c2ccc(-c3ccc(-c4ccc5ccccc5c4)cc3)cc2)c2cccc3oc4c5ccccc5ccc4c23)c(-c2ccc3c(c2)oc2ccccc23)c1 ; c1ccc(N(c2ccc(-c3ccc(-c4cccc5ccccc45)cc3)cc2)c2cccc3oc4c5ccccc5ccc4c23)c(-c2ccc3c(c2)oc2ccccc23)c1 ; c1cc(-c2ccc(N(c3ccccc3-c3ccc4c(c3)oc3ccccc34)c3cccc4oc5c6ccccc6ccc5c34)cc2)cc(-c2cccc3ccccc23)c1]

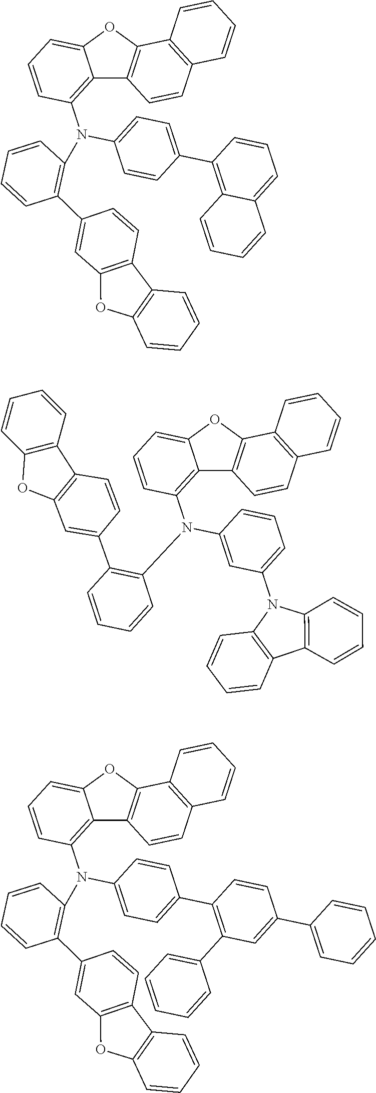 [SMILES: c1ccc(N(c2ccc(-c3cccc4ccccc34)cc2)c2cccc3oc4c5ccccc5ccc4c23)c(-c2ccc3c(c2)oc2ccccc23)c1 ; c1cc(N(c2ccccc2-c2ccc3c(c2)oc2ccccc23)c2cccc3oc4c5ccccc5ccc4c23)cc(-n2c3ccccc3c3ccccc32)c1 ; c1ccc(-c2ccc(-c3ccc(N(c4ccccc4-c4ccc5c(c4)oc4ccccc45)c4cccc5oc6c7ccccc7ccc6c45)cc3)c(-c3ccccc3)c2)cc1]

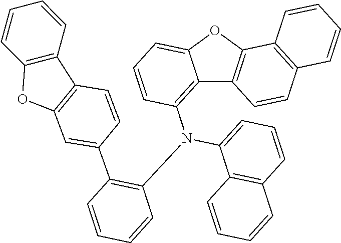 [SMILES: c1ccc(N(c2cccc3ccccc23)c2cccc3oc4c5ccccc5ccc4c23)c(-c2ccc3c(c2)oc2ccccc23)c1]

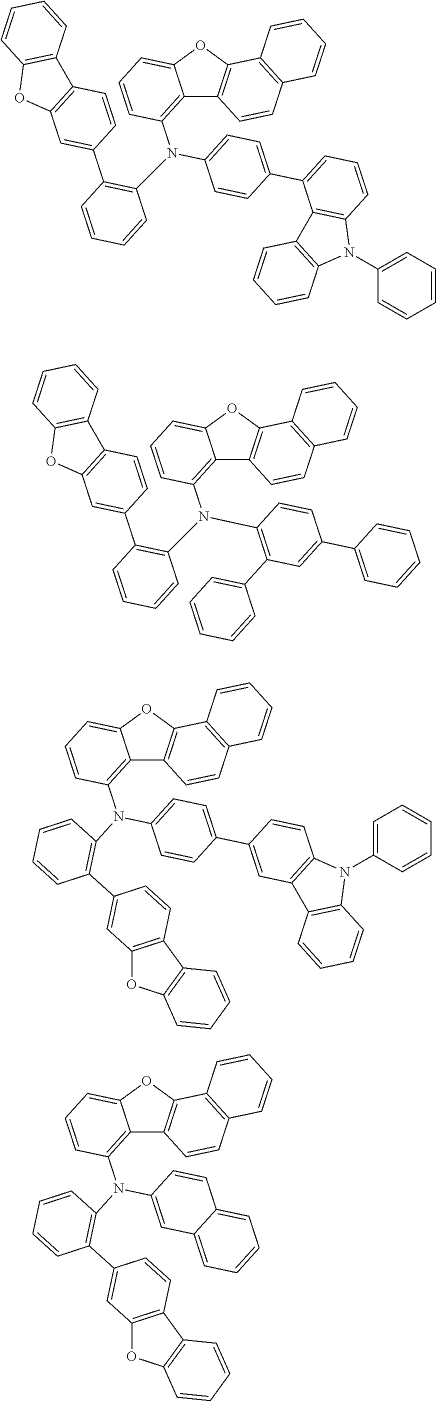

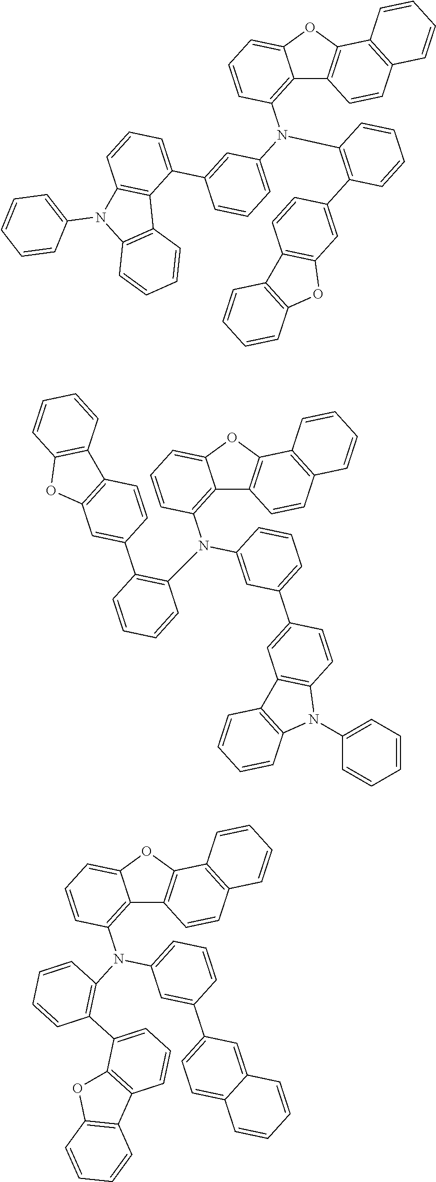 [SMILES: c1ccc(-n2c3ccccc3c3c(-c4cccc(N(c5ccccc5-c5ccc6c(c5)oc5ccccc56)c5cccc6oc7c8ccccc8ccc7c56)c4)cccc32)cc1 ; c1ccc(-n2c3ccccc3c3cc(-c4cccc(N(c5ccccc5-c5ccc6c(c5)oc5ccccc56)c5cccc6oc7c8ccccc8ccc7c56)c4)ccc32)cc1 ; c1cc(-c2ccc3ccccc3c2)cc(N(c2ccccc2-c2cccc3c2oc2ccccc23)c2cccc3oc4c5ccccc5ccc4c23)c1]

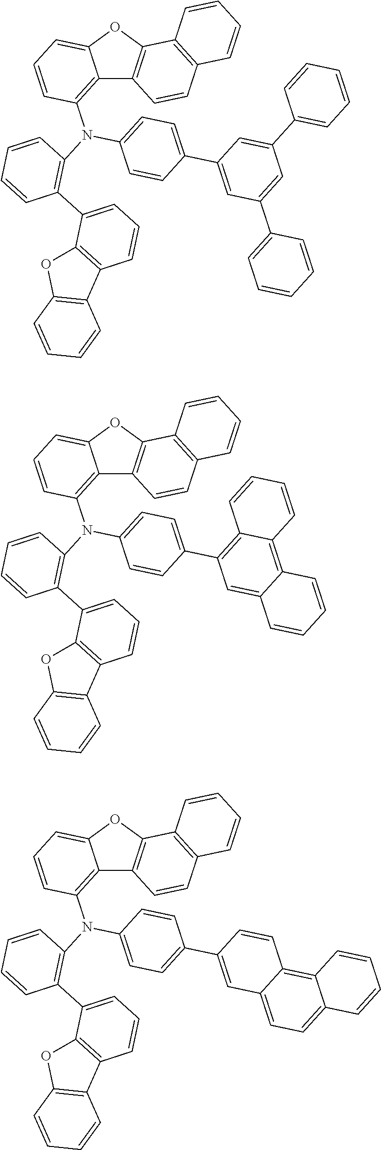 [SMILES: c1ccc(-c2cc(-c3ccccc3)cc(-c3ccc(N(c4ccccc4-c4cccc5c4oc4ccccc45)c4cccc5oc6c7ccccc7ccc6c45)cc3)c2)cc1 ; c1ccc(N(c2ccc(-c3cc4ccccc4c4ccccc34)cc2)c2cccc3oc4c5ccccc5ccc4c23)c(-c2cccc3c2oc2ccccc23)c1 ; c1ccc(N(c2ccc(-c3ccc4c(ccc5ccccc54)c3)cc2)c2cccc3oc4c5ccccc5ccc4c23)c(-c2cccc3c2oc2ccccc23)c1]

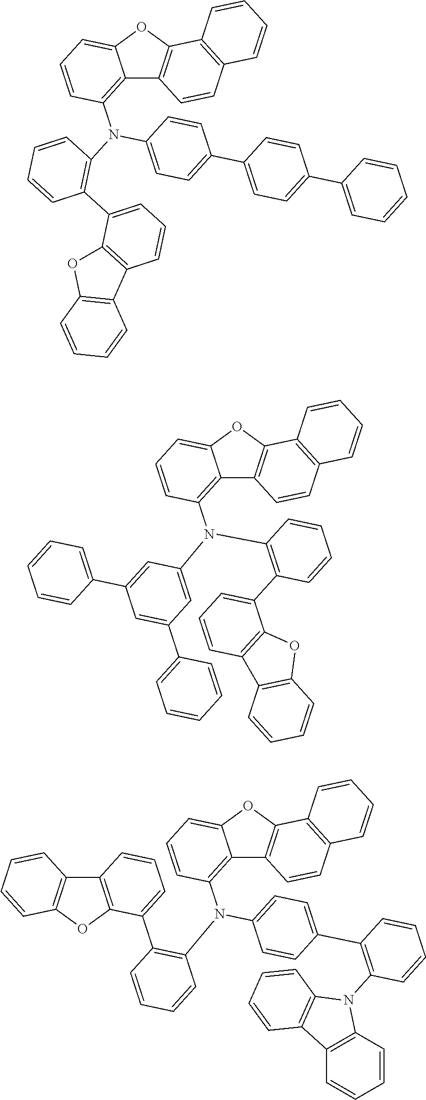 [SMILES: c1ccc(-c2ccc(-c3ccc(N(c4ccccc4-c4cccc5c4oc4ccccc45)c4cccc5oc6c7ccccc7ccc6c45)cc3)cc2)cc1 ; c1ccc(-c2cc(-c3ccccc3)cc(N(c3ccccc3-c3cccc4c3oc3ccccc34)c3cccc4oc5c6ccccc6ccc5c34)c2)cc1 ; c1ccc(N(c2ccc(-c3ccccc3-n3c4ccccc4c4ccccc43)cc2)c2cccc3oc4c5ccccc5ccc4c23)c(-c2cccc3c2oc2ccccc23)c1]

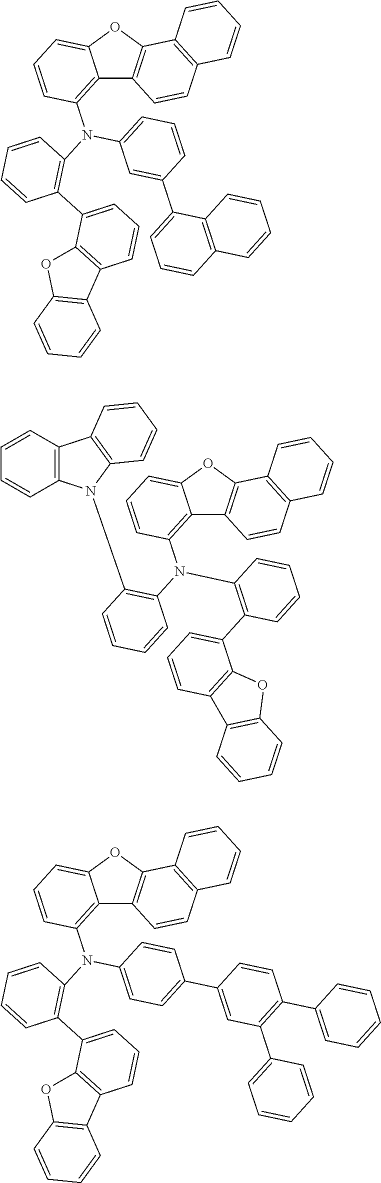 [SMILES: c1cc(-c2cccc3ccccc23)cc(N(c2ccccc2-c2cccc3c2oc2ccccc23)c2cccc3oc4c5ccccc5ccc4c23)c1 ; c1ccc(N(c2ccccc2-n2c3ccccc3c3ccccc32)c2cccc3oc4c5ccccc5ccc4c23)c(-c2cccc3c2oc2ccccc23)c1 ; c1ccc(-c2ccc(-c3ccc(N(c4ccccc4-c4cccc5c4oc4ccccc45)c4cccc5oc6c7ccccc7ccc6c45)cc3)cc2-c2ccccc2)cc1]

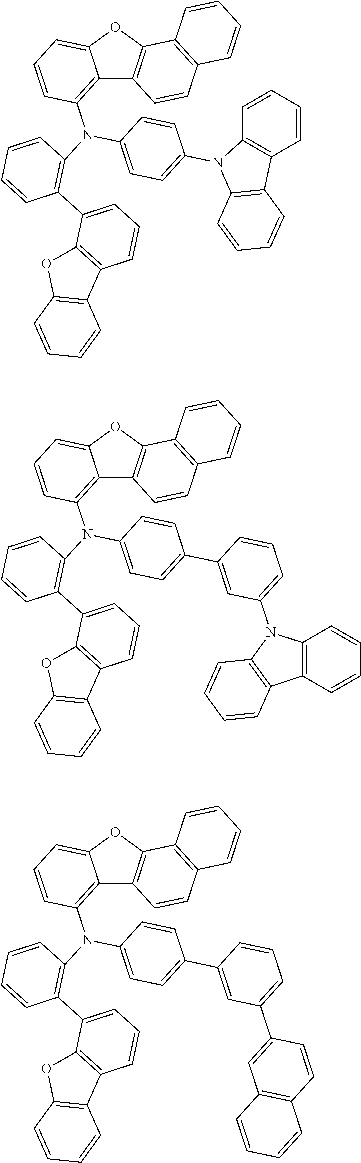 [SMILES: c1ccc(N(c2ccc(-n3c4ccccc4c4ccccc43)cc2)c2cccc3oc4c5ccccc5ccc4c23)c(-c2cccc3c2oc2ccccc23)c1 ; c1cc(-c2ccc(N(c3ccccc3-c3cccc4c3oc3ccccc34)c3cccc4oc5c6ccccc6ccc5c34)cc2)cc(-n2c3ccccc3c3ccccc32)c1 ; c1cc(-c2ccc(N(c3ccccc3-c3cccc4c3oc3ccccc34)c3cccc4oc5c6ccccc6ccc5c34)cc2)cc(-c2ccc3ccccc3c2)c1]

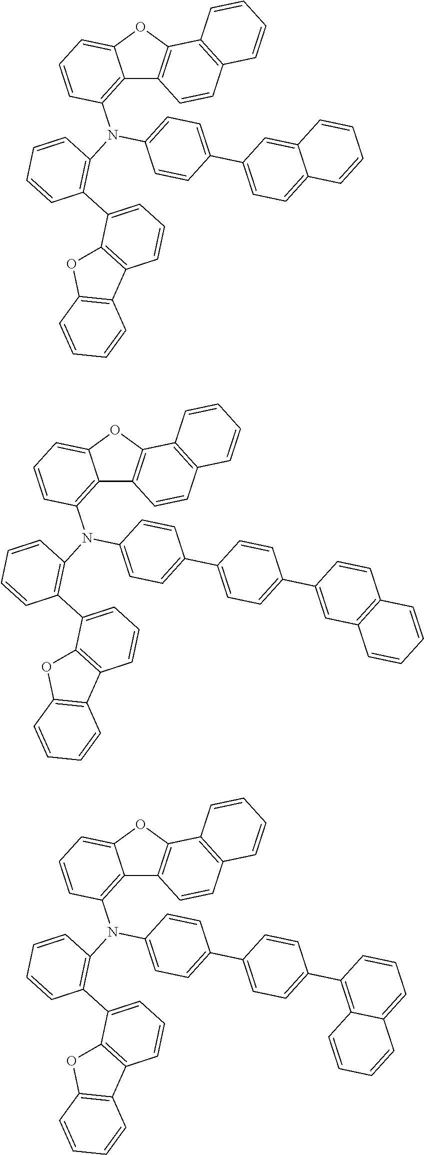 [SMILES: c1ccc(N(c2ccc(-c3ccc4ccccc4c3)cc2)c2cccc3oc4c5ccccc5ccc4c23)c(-c2cccc3c2oc2ccccc23)c1 ; c1ccc(N(c2ccc(-c3ccc(-c4ccc5ccccc5c4)cc3)cc2)c2cccc3oc4c5ccccc5ccc4c23)c(-c2cccc3c2oc2ccccc23)c1 ; c1ccc(N(c2ccc(-c3ccc(-c4cccc5ccccc45)cc3)cc2)c2cccc3oc4c5ccccc5ccc4c23)c(-c2cccc3c2oc2ccccc23)c1]

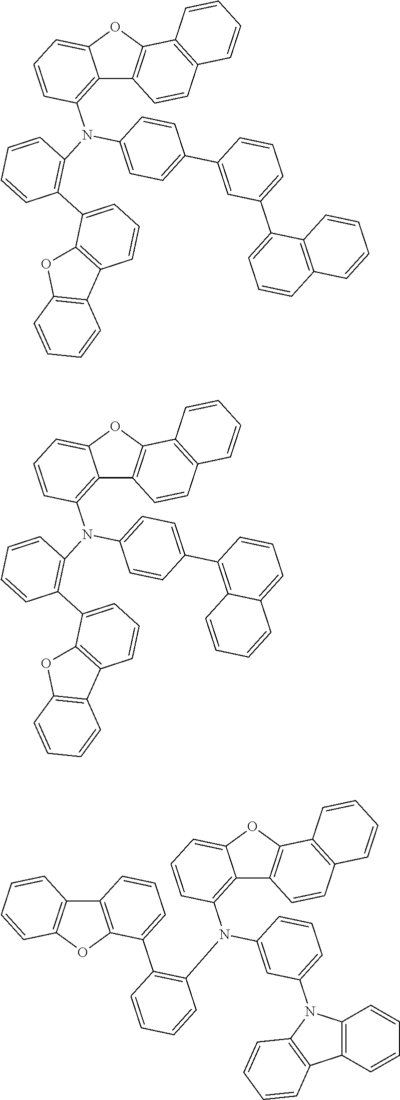 [SMILES: c1cc(-c2ccc(N(c3ccccc3-c3cccc4c3oc3ccccc34)c3cccc4oc5c6ccccc6ccc5c34)cc2)cc(-c2cccc3ccccc23)c1 ; c1ccc(N(c2ccc(-c3cccc4ccccc34)cc2)c2cccc3oc4c5ccccc5ccc4c23)c(-c2cccc3c2oc2ccccc23)c1 ; c1cc(N(c2ccccc2-c2cccc3c2oc2ccccc23)c2cccc3oc4c5ccccc5ccc4c23)cc(-n2c3ccccc3c3ccccc32)c1]

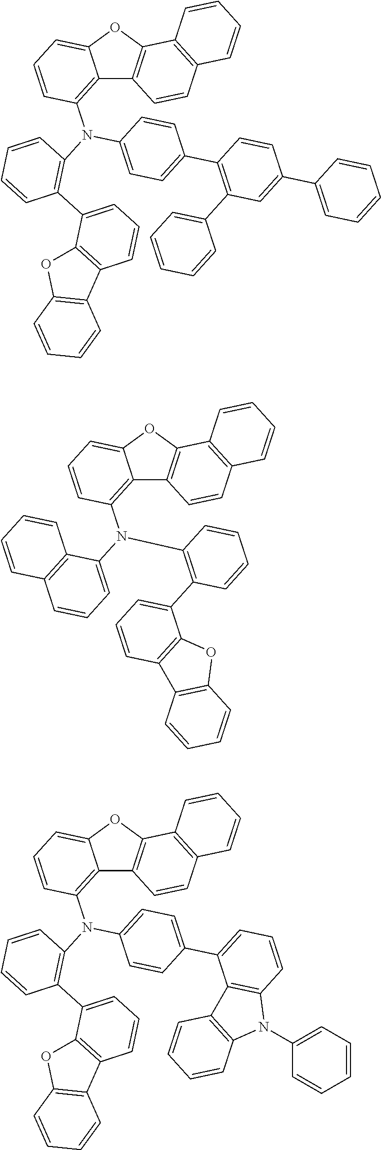 [SMILES: c1ccc(-c2ccc(-c3ccc(N(c4ccccc4-c4cccc5c4oc4ccccc45)c4cccc5oc6c7ccccc7ccc6c45)cc3)c(-c3ccccc3)c2)cc1 ; c1ccc(N(c2cccc3ccccc23)c2cccc3oc4c5ccccc5ccc4c23)c(-c2cccc3c2oc2ccccc23)c1 ; c1ccc(-n2c3ccccc3c3c(-c4ccc(N(c5ccccc5-c5cccc6c5oc5ccccc56)c5cccc6oc7c8ccccc8ccc7c56)cc4)cccc32)cc1]

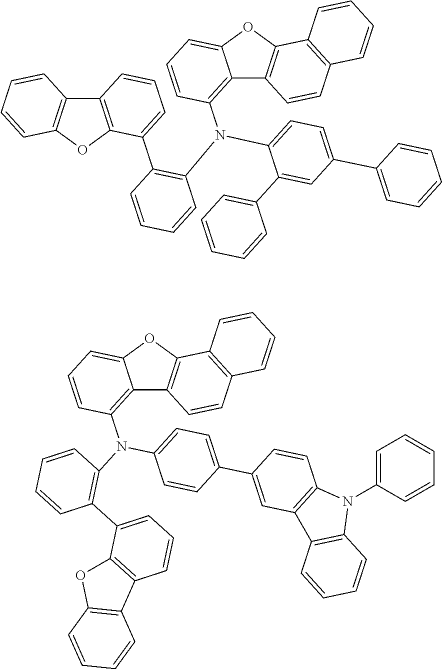 [SMILES: c1ccc(-c2ccc(N(c3ccccc3-c3cccc4c3oc3ccccc34)c3cccc4oc5c6ccccc6ccc5c34)c(-c3ccccc3)c2)cc1 ; c1ccc(-n2c3ccccc3c3cc(-c4ccc(N(c5ccccc5-c5cccc6c5oc5ccccc56)c5cccc6oc7c8ccccc8ccc7c56)cc4)ccc32)cc1]

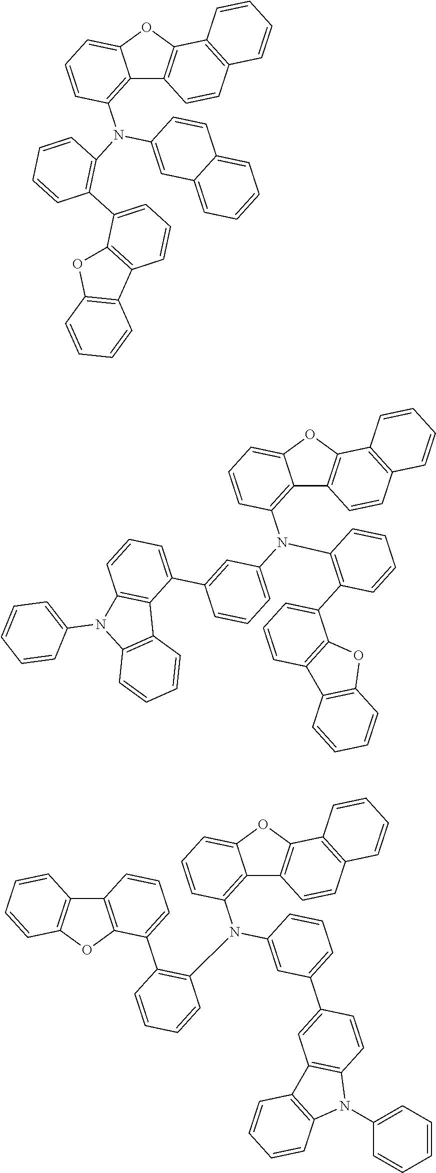 [SMILES: c1ccc(N(c2ccc3ccccc3c2)c2cccc3oc4c5ccccc5ccc4c23)c(-c2cccc3c2oc2ccccc23)c1 ; c1ccc(-n2c3ccccc3c3c(-c4cccc(N(c5ccccc5-c5cccc6c5oc5ccccc56)c5cccc6oc7c8ccccc8ccc7c56)c4)cccc32)cc1 ; c1ccc(-n2c3ccccc3c3cc(-c4cccc(N(c5ccccc5-c5cccc6c5oc5ccccc56)c5cccc6oc7c8ccccc8ccc7c56)c4)ccc32)cc1]

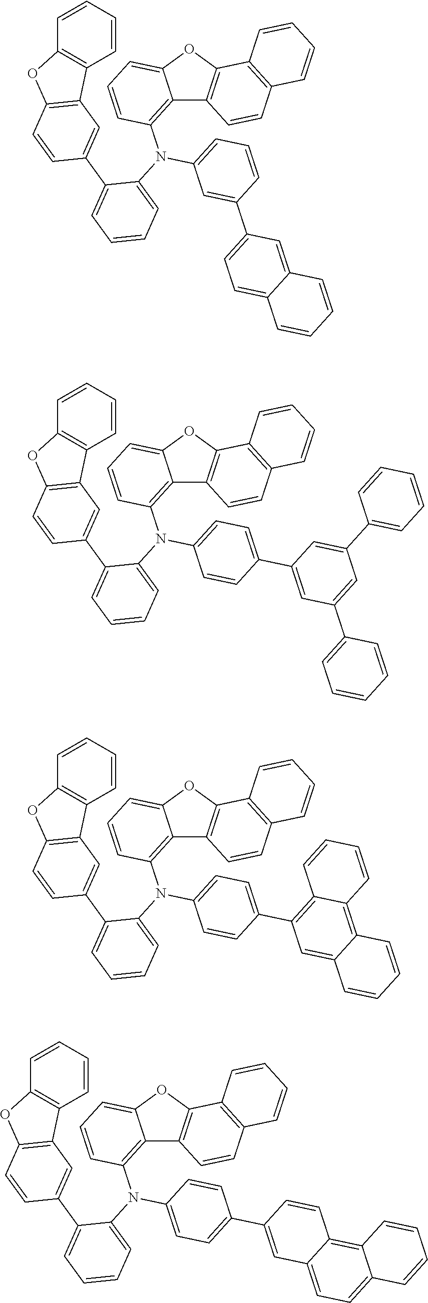 [SMILES: c1cc(-c2ccc3ccccc3c2)cc(N(c2ccccc2-c2ccc3oc4ccccc4c3c2)c2cccc3oc4c5ccccc5ccc4c23)c1 ; c1ccc(-c2cc(-c3ccccc3)cc(-c3ccc(N(c4ccccc4-c4ccc5oc6ccccc6c5c4)c4cccc5oc6c7ccccc7ccc6c45)cc3)c2)cc1 ; c1ccc(N(c2ccc(-c3cc4ccccc4c4ccccc34)cc2)c2cccc3oc4c5ccccc5ccc4c23)c(-c2ccc3oc4ccccc4c3c2)c1 ; c1ccc(N(c2ccc(-c3ccc4c(ccc5ccccc54)c3)cc2)c2cccc3oc4c5ccccc5ccc4c23)c(-c2ccc3oc4ccccc4c3c2)c1]

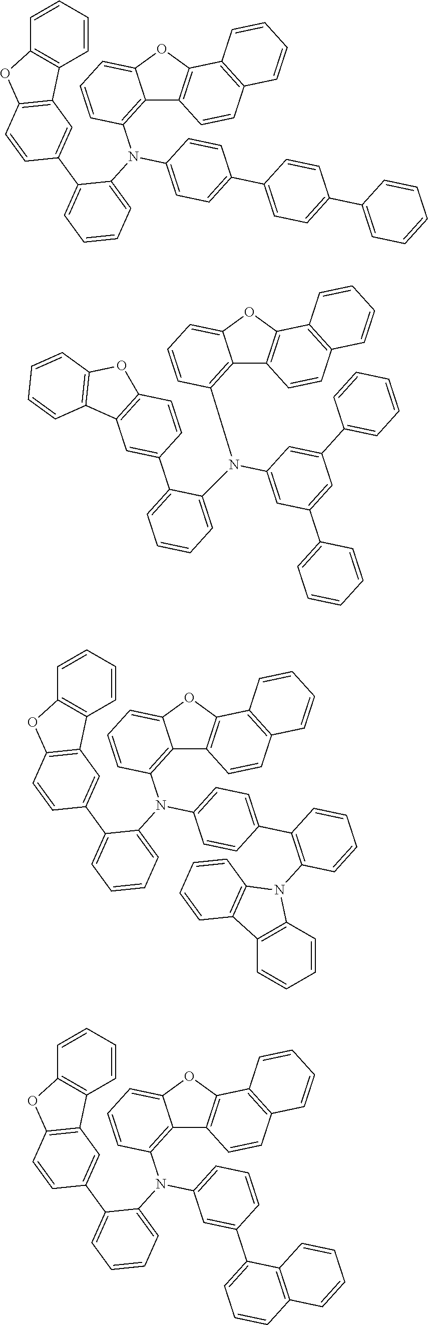 [SMILES: c1ccc(-c2ccc(-c3ccc(N(c4ccccc4-c4ccc5oc6ccccc6c5c4)c4cccc5oc6c7ccccc7ccc6c45)cc3)cc2)cc1 ; c1ccc(-c2cc(-c3ccccc3)cc(N(c3ccccc3-c3ccc4oc5ccccc5c4c3)c3cccc4oc5c6ccccc6ccc5c34)c2)cc1 ; c1ccc(N(c2ccc(-c3ccccc3-n3c4ccccc4c4ccccc43)cc2)c2cccc3oc4c5ccccc5ccc4c23)c(-c2ccc3oc4ccccc4c3c2)c1 ; c1cc(-c2cccc3ccccc23)cc(N(c2ccccc2-c2ccc3oc4ccccc4c3c2)c2cccc3oc4c5ccccc5ccc4c23)c1]

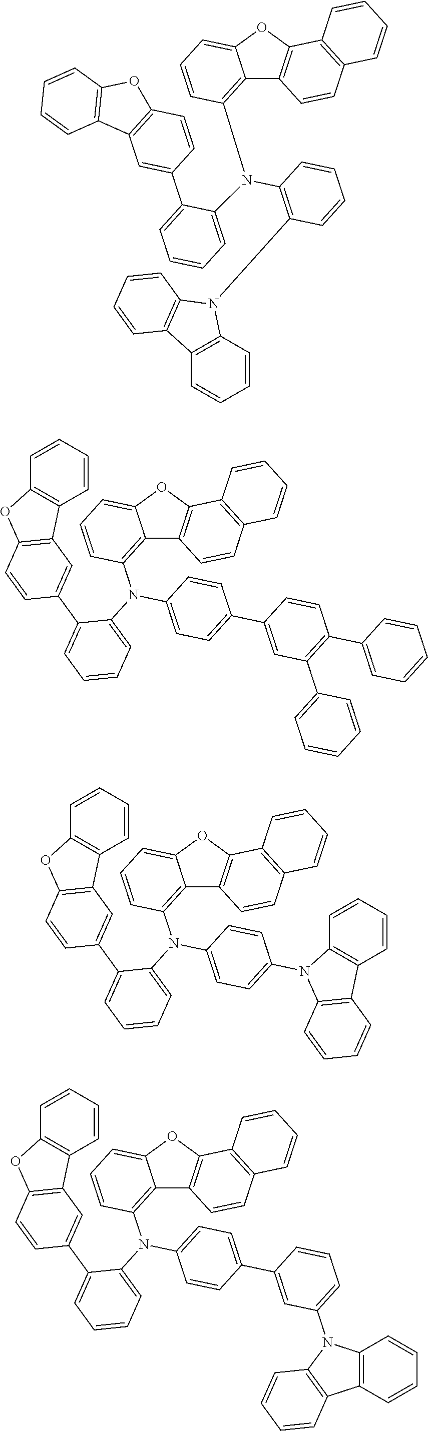 [SMILES: c1ccc(N(c2ccccc2-n2c3ccccc3c3ccccc32)c2cccc3oc4c5ccccc5ccc4c23)c(-c2ccc3oc4ccccc4c3c2)c1 ; c1ccc(-c2ccc(-c3ccc(N(c4ccccc4-c4ccc5oc6ccccc6c5c4)c4cccc5oc6c7ccccc7ccc6c45)cc3)cc2-c2ccccc2)cc1 ; c1ccc(N(c2ccc(-n3c4ccccc4c4ccccc43)cc2)c2cccc3oc4c5ccccc5ccc4c23)c(-c2ccc3oc4ccccc4c3c2)c1 ; c1cc(-c2ccc(N(c3ccccc3-c3ccc4oc5ccccc5c4c3)c3cccc4oc5c6ccccc6ccc5c34)cc2)cc(-n2c3ccccc3c3ccccc32)c1]

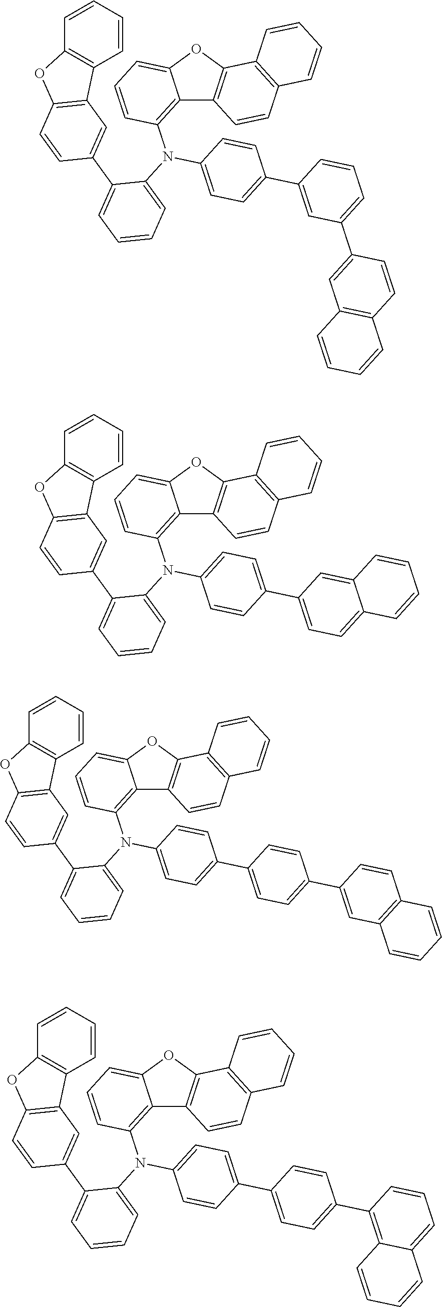 [SMILES: c1cc(-c2ccc(N(c3ccccc3-c3ccc4oc5ccccc5c4c3)c3cccc4oc5c6ccccc6ccc5c34)cc2)cc(-c2ccc3ccccc3c2)c1 ; c1ccc(N(c2ccc(-c3ccc4ccccc4c3)cc2)c2cccc3oc4c5ccccc5ccc4c23)c(-c2ccc3oc4ccccc4c3c2)c1 ; c1ccc(N(c2ccc(-c3ccc(-c4ccc5ccccc5c4)cc3)cc2)c2cccc3oc4c5ccccc5ccc4c23)c(-c2ccc3oc4ccccc4c3c2)c1 ; c1ccc(N(c2ccc(-c3ccc(-c4cccc5ccccc45)cc3)cc2)c2cccc3oc4c5ccccc5ccc4c23)c(-c2ccc3oc4ccccc4c3c2)c1]

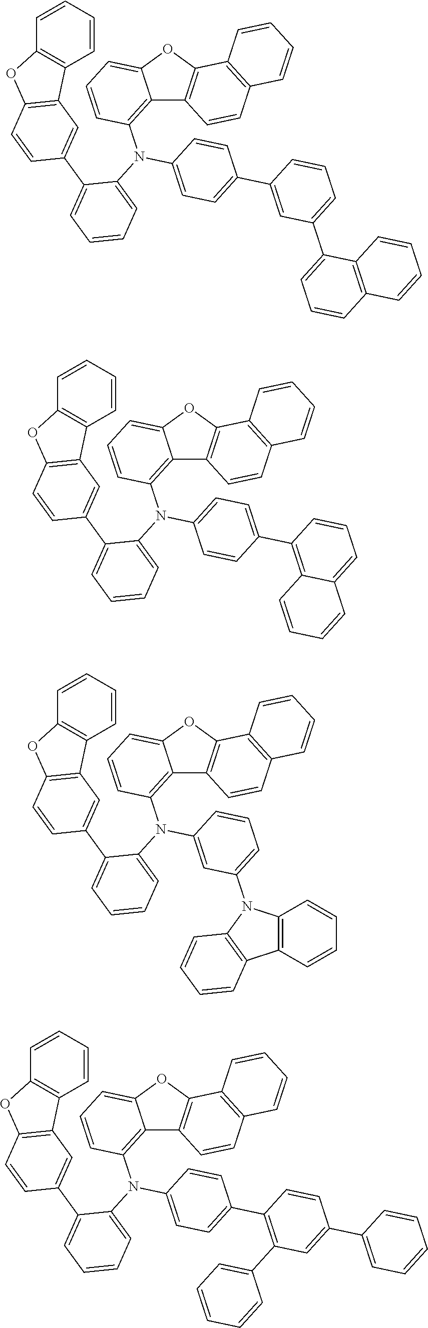 [SMILES: c1cc(-c2ccc(N(c3ccccc3-c3ccc4oc5ccccc5c4c3)c3cccc4oc5c6ccccc6ccc5c34)cc2)cc(-c2cccc3ccccc23)c1 ; c1ccc(N(c2ccc(-c3cccc4ccccc34)cc2)c2cccc3oc4c5ccccc5ccc4c23)c(-c2ccc3oc4ccccc4c3c2)c1 ; c1cc(N(c2ccccc2-c2ccc3oc4ccccc4c3c2)c2cccc3oc4c5ccccc5ccc4c23)cc(-n2c3ccccc3c3ccccc32)c1 ; c1ccc(-c2ccc(-c3ccc(N(c4ccccc4-c4ccc5oc6ccccc6c5c4)c4cccc5oc6c7ccccc7ccc6c45)cc3)c(-c3ccccc3)c2)cc1]

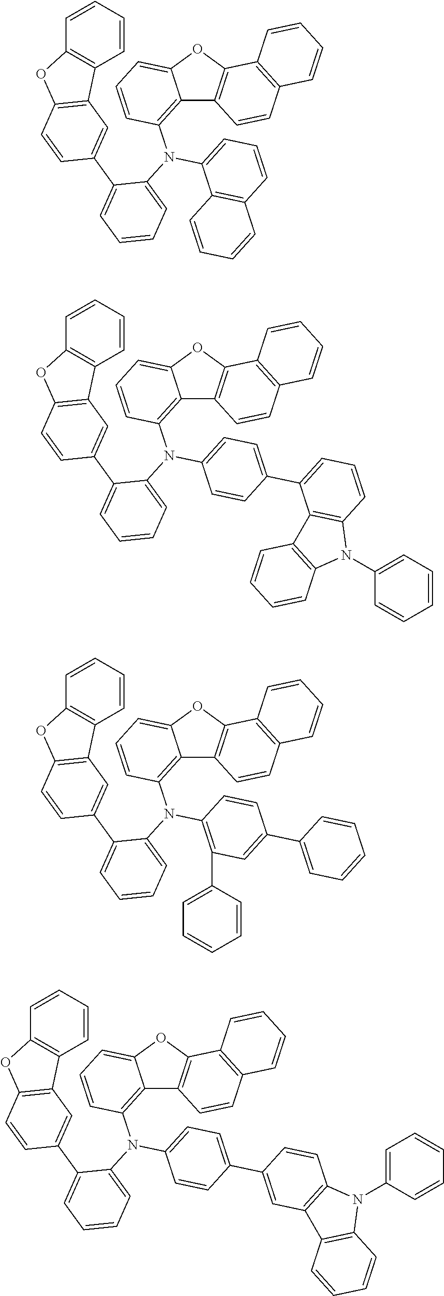 [SMILES: c1ccc(N(c2cccc3ccccc23)c2cccc3oc4c5ccccc5ccc4c23)c(-c2ccc3oc4ccccc4c3c2)c1 ; c1ccc(-n2c3ccccc3c3c(-c4ccc(N(c5ccccc5-c5ccc6oc7ccccc7c6c5)c5cccc6oc7c8ccccc8ccc7c56)cc4)cccc32)cc1 ; c1ccc(-c2ccc(N(c3ccccc3-c3ccc4oc5ccccc5c4c3)c3cccc4oc5c6ccccc6ccc5c34)c(-c3ccccc3)c2)cc1 ; c1ccc(-n2c3ccccc3c3cc(-c4ccc(N(c5ccccc5-c5ccc6oc7ccccc7c6c5)c5cccc6oc7c8ccccc8ccc7c56)cc4)ccc32)cc1]

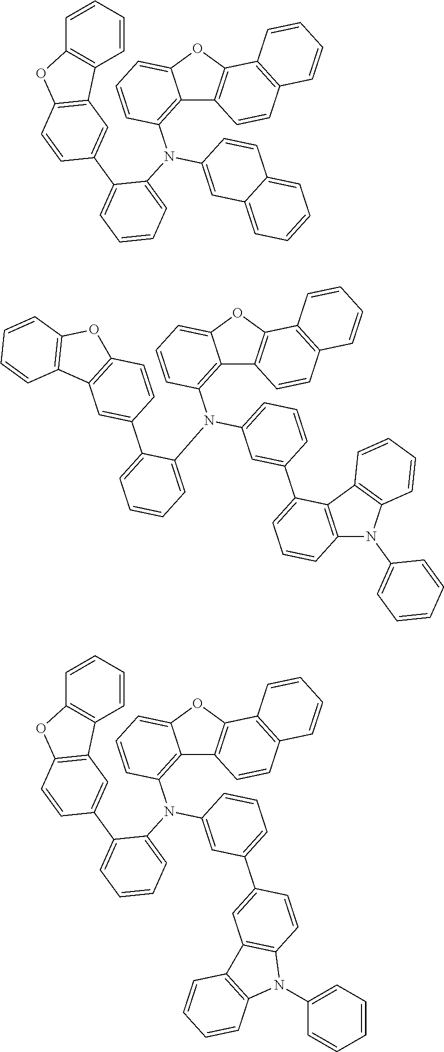 [SMILES: c1ccc(N(c2ccc3ccccc3c2)c2cccc3oc4c5ccccc5ccc4c23)c(-c2ccc3oc4ccccc4c3c2)c1 ; c1ccc(-n2c3ccccc3c3c(-c4cccc(N(c5ccccc5-c5ccc6oc7ccccc7c6c5)c5cccc6oc7c8ccccc8ccc7c56)c4)cccc32)cc1 ; c1ccc(-n2c3ccccc3c3cc(-c4cccc(N(c5ccccc5-c5ccc6oc7ccccc7c6c5)c5cccc6oc7c8ccccc8ccc7c56)c4)ccc32)cc1]

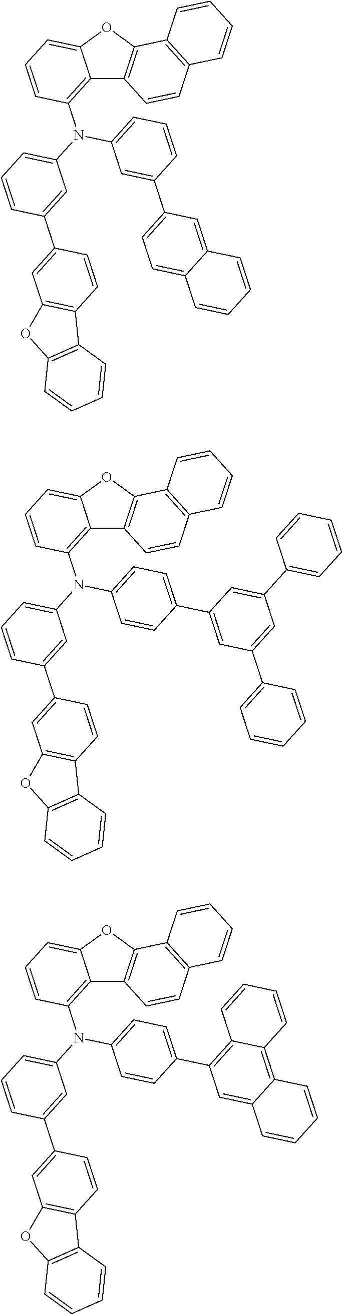 [SMILES: c1cc(-c2ccc3ccccc3c2)cc(N(c2cccc(-c3ccc4c(c3)oc3ccccc34)c2)c2cccc3oc4c5ccccc5ccc4c23)c1 ; c1ccc(-c2cc(-c3ccccc3)cc(-c3ccc(N(c4cccc(-c5ccc6c(c5)oc5ccccc56)c4)c4cccc5oc6c7ccccc7ccc6c45)cc3)c2)cc1 ; c1cc(-c2ccc3c(c2)oc2ccccc23)cc(N(c2ccc(-c3cc4ccccc4c4ccccc34)cc2)c2cccc3oc4c5ccccc5ccc4c23)c1]

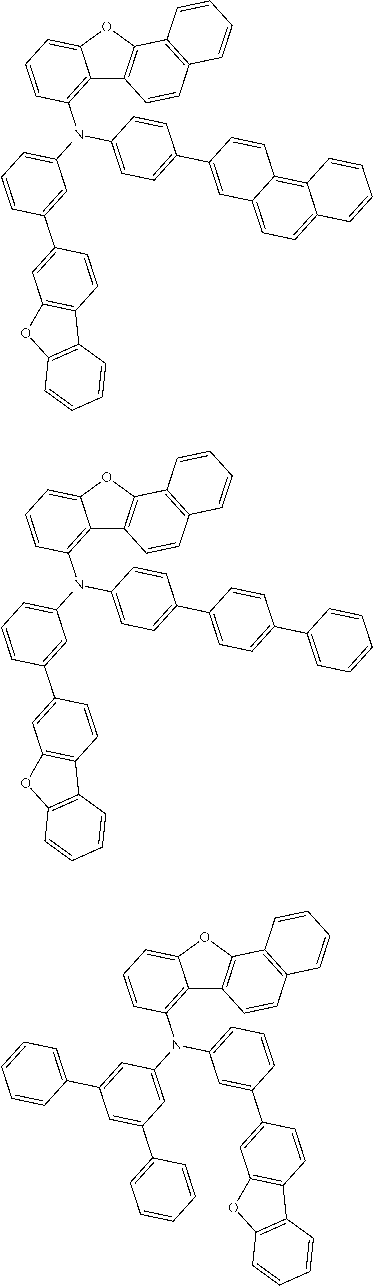 [SMILES: c1cc(-c2ccc3c(c2)oc2ccccc23)cc(N(c2ccc(-c3ccc4c(ccc5ccccc54)c3)cc2)c2cccc3oc4c5ccccc5ccc4c23)c1 ; c1ccc(-c2ccc(-c3ccc(N(c4cccc(-c5ccc6c(c5)oc5ccccc56)c4)c4cccc5oc6c7ccccc7ccc6c45)cc3)cc2)cc1 ; c1ccc(-c2cc(-c3ccccc3)cc(N(c3cccc(-c4ccc5c(c4)oc4ccccc45)c3)c3cccc4oc5c6ccccc6ccc5c34)c2)cc1]

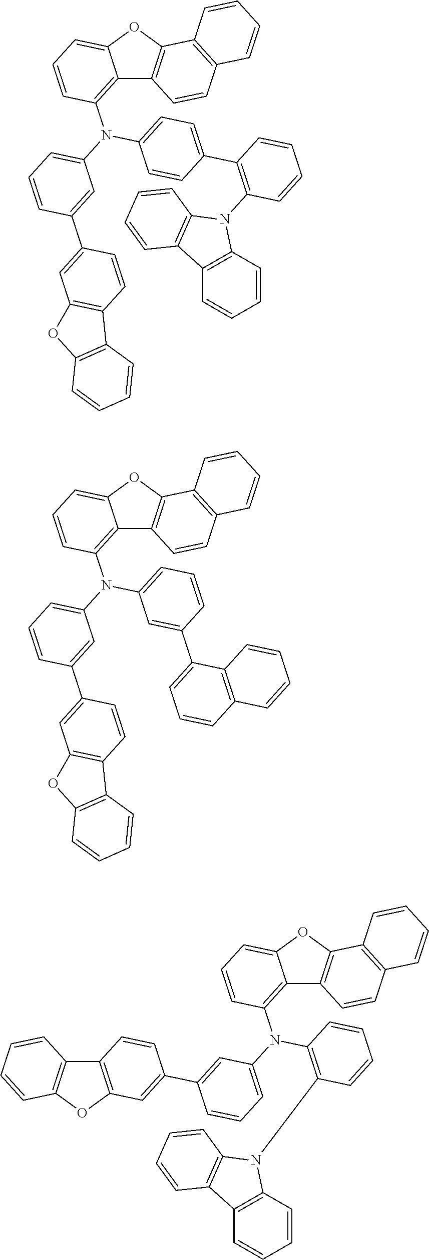 [SMILES: c1cc(-c2ccc3c(c2)oc2ccccc23)cc(N(c2ccc(-c3ccccc3-n3c4ccccc4c4ccccc43)cc2)c2cccc3oc4c5ccccc5ccc4c23)c1 ; c1cc(-c2ccc3c(c2)oc2ccccc23)cc(N(c2cccc(-c3cccc4ccccc34)c2)c2cccc3oc4c5ccccc5ccc4c23)c1 ; c1cc(-c2ccc3c(c2)oc2ccccc23)cc(N(c2ccccc2-n2c3ccccc3c3ccccc32)c2cccc3oc4c5ccccc5ccc4c23)c1]

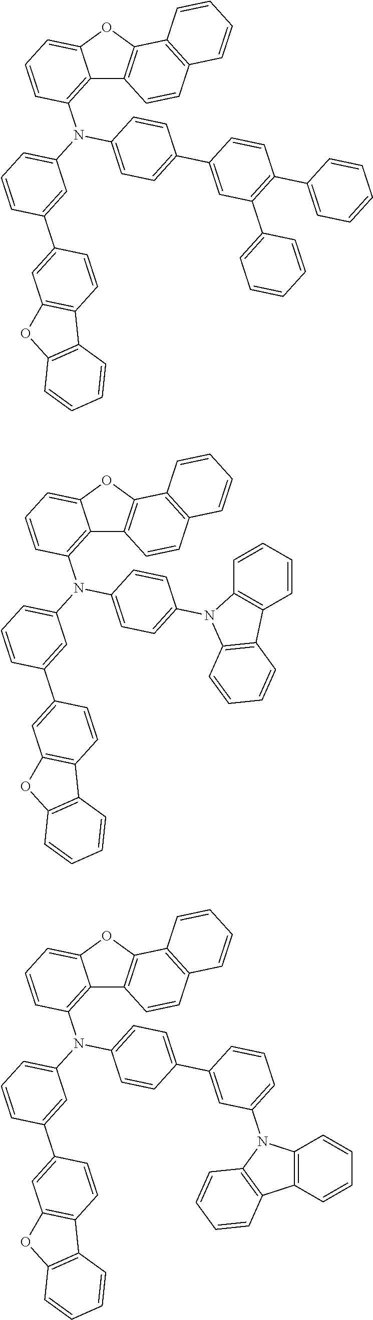 [SMILES: c1ccc(-c2ccc(-c3ccc(N(c4cccc(-c5ccc6c(c5)oc5ccccc56)c4)c4cccc5oc6c7ccccc7ccc6c45)cc3)cc2-c2ccccc2)cc1 ; c1cc(-c2ccc3c(c2)oc2ccccc23)cc(N(c2ccc(-n3c4ccccc4c4ccccc43)cc2)c2cccc3oc4c5ccccc5ccc4c23)c1 ; c1cc(-c2ccc3c(c2)oc2ccccc23)cc(N(c2ccc(-c3cccc(-n4c5ccccc5c5ccccc54)c3)cc2)c2cccc3oc4c5ccccc5ccc4c23)c1]

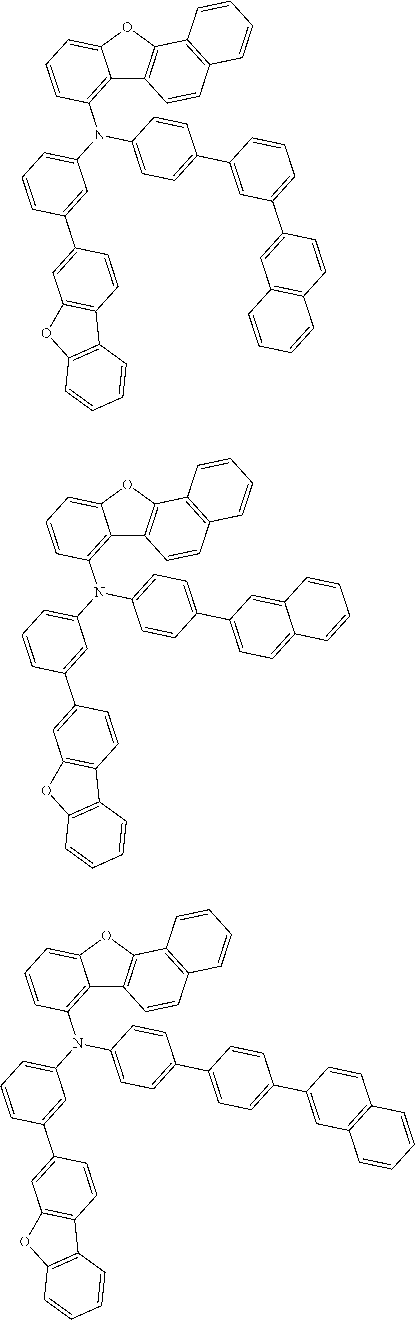 [SMILES: c1cc(-c2ccc(N(c3cccc(-c4ccc5c(c4)oc4ccccc45)c3)c3cccc4oc5c6ccccc6ccc5c34)cc2)cc(-c2ccc3ccccc3c2)c1 ; c1cc(-c2ccc3c(c2)oc2ccccc23)cc(N(c2ccc(-c3ccc4ccccc4c3)cc2)c2cccc3oc4c5ccccc5ccc4c23)c1 ; c1cc(-c2ccc3c(c2)oc2ccccc23)cc(N(c2ccc(-c3ccc(-c4ccc5ccccc5c4)cc3)cc2)c2cccc3oc4c5ccccc5ccc4c23)c1]

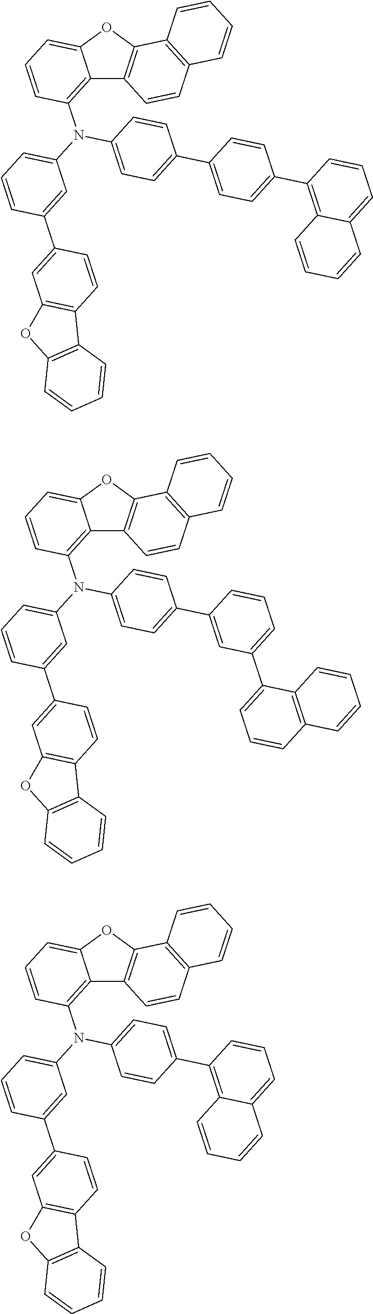 [SMILES: c1cc(-c2ccc3c(c2)oc2ccccc23)cc(N(c2ccc(-c3ccc(-c4cccc5ccccc45)cc3)cc2)c2cccc3oc4c5ccccc5ccc4c23)c1 ; c1cc(-c2ccc(N(c3cccc(-c4ccc5c(c4)oc4ccccc45)c3)c3cccc4oc5c6ccccc6ccc5c34)cc2)cc(-c2cccc3ccccc23)c1 ; c1cc(-c2ccc3c(c2)oc2ccccc23)cc(N(c2ccc(-c3cccc4ccccc34)cc2)c2cccc3oc4c5ccccc5ccc4c23)c1]

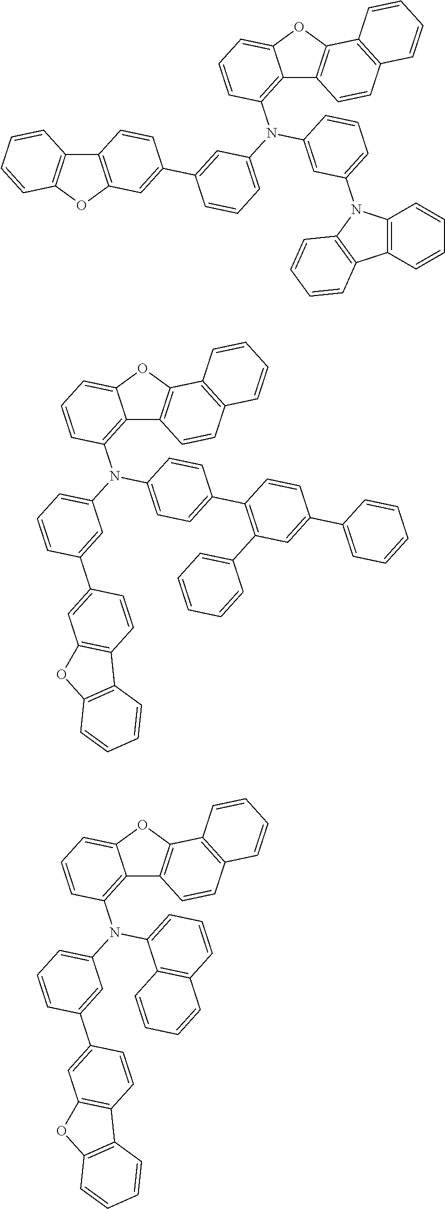 [SMILES: c1cc(-c2ccc3c(c2)oc2ccccc23)cc(N(c2cccc(-n3c4ccccc4c4ccccc43)c2)c2cccc3oc4c5ccccc5ccc4c23)c1 ; c1ccc(-c2ccc(-c3ccc(N(c4cccc(-c5ccc6c(c5)oc5ccccc56)c4)c4cccc5oc6c7ccccc7ccc6c45)cc3)c(-c3ccccc3)c2)cc1 ; c1cc(-c2ccc3c(c2)oc2ccccc23)cc(N(c2cccc3ccccc23)c2cccc3oc4c5ccccc5ccc4c23)c1]

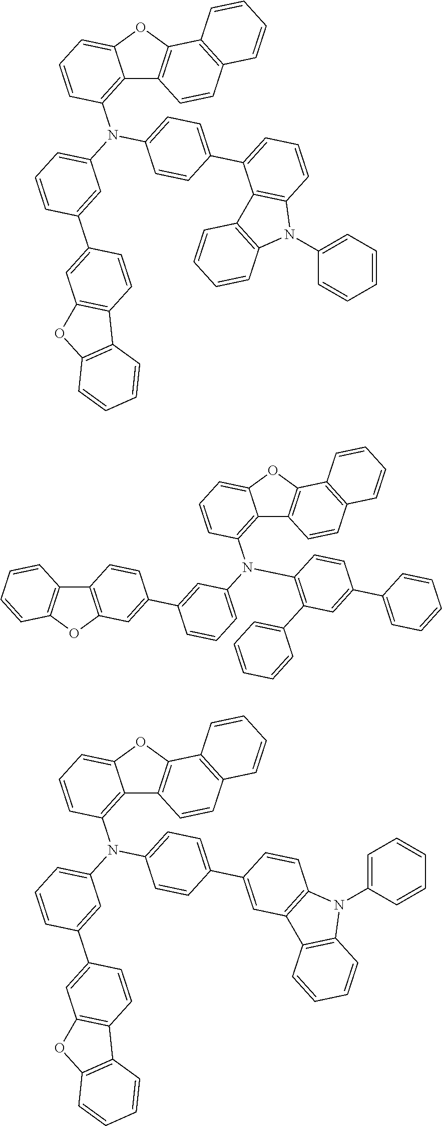

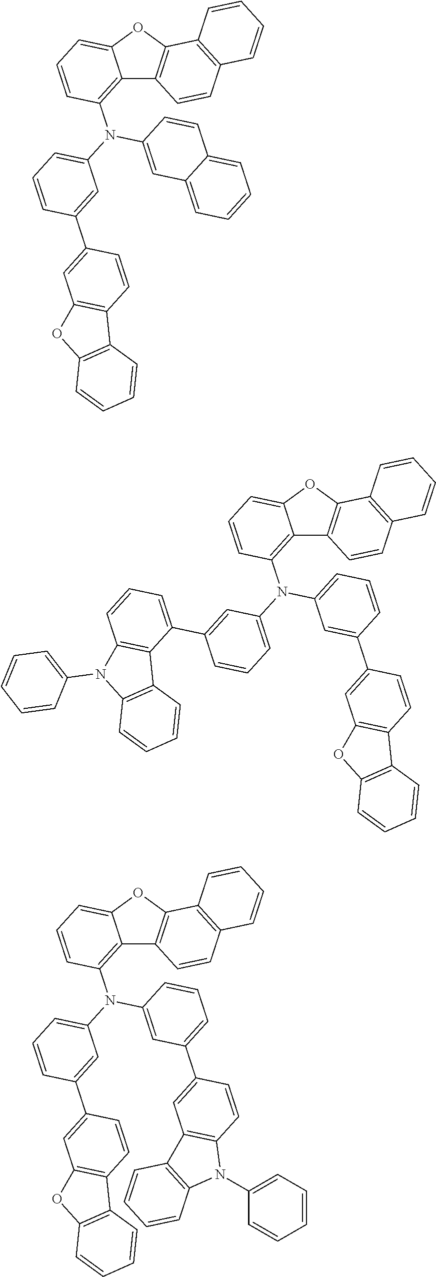 [SMILES: c1cc(-c2ccc3c(c2)oc2ccccc23)cc(N(c2ccc3ccccc3c2)c2cccc3oc4c5ccccc5ccc4c23)c1 ; c1ccc(-n2c3ccccc3c3c(-c4cccc(N(c5cccc(-c6ccc7c(c6)oc6ccccc67)c5)c5cccc6oc7c8ccccc8ccc7c56)c4)cccc32)cc1 ; c1ccc(-n2c3ccccc3c3cc(-c4cccc(N(c5cccc(-c6ccc7c(c6)oc6ccccc67)c5)c5cccc6oc7c8ccccc8ccc7c56)c4)ccc32)cc1]

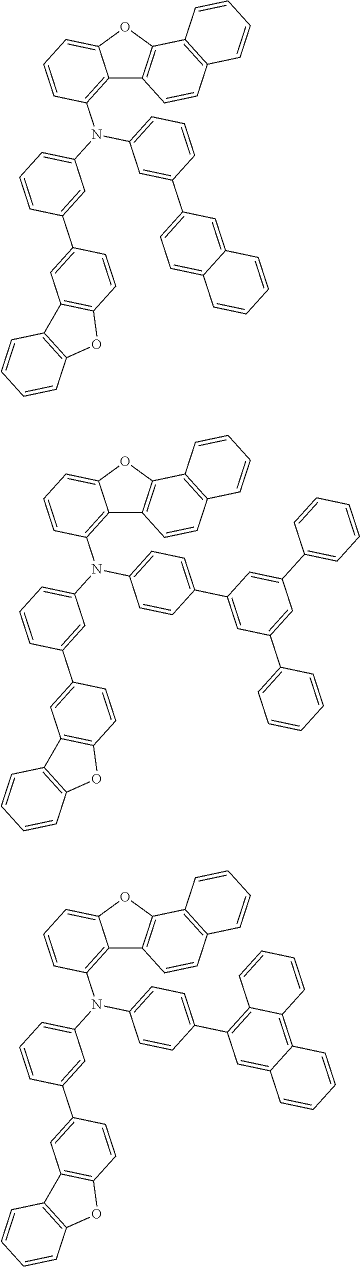 [SMILES: c1cc(-c2ccc3ccccc3c2)cc(N(c2cccc(-c3ccc4oc5ccccc5c4c3)c2)c2cccc3oc4c5ccccc5ccc4c23)c1 ; c1ccc(-c2cc(-c3ccccc3)cc(-c3ccc(N(c4cccc(-c5ccc6oc7ccccc7c6c5)c4)c4cccc5oc6c7ccccc7ccc6c45)cc3)c2)cc1 ; c1cc(-c2ccc3oc4ccccc4c3c2)cc(N(c2ccc(-c3cc4ccccc4c4ccccc34)cc2)c2cccc3oc4c5ccccc5ccc4c23)c1]

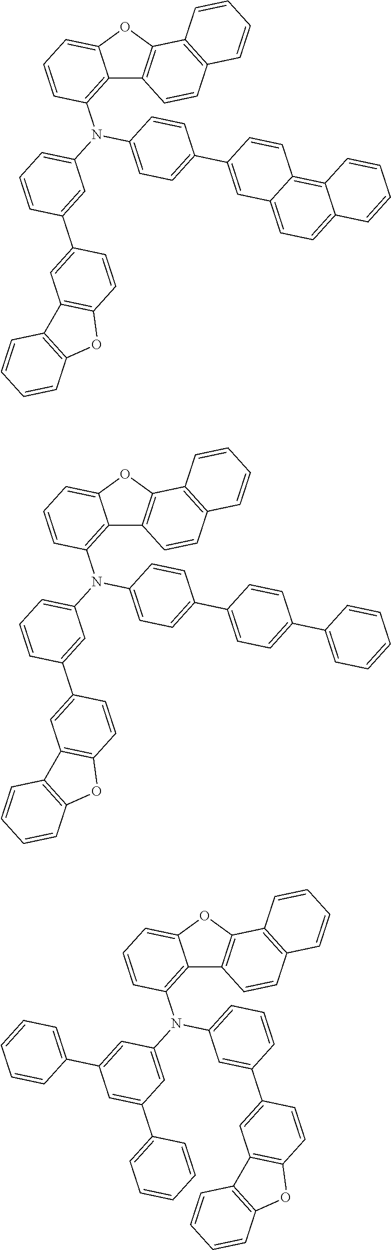 [SMILES: c1cc(-c2ccc3oc4ccccc4c3c2)cc(N(c2ccc(-c3ccc4c(ccc5ccccc54)c3)cc2)c2cccc3oc4c5ccccc5ccc4c23)c1 ; c1ccc(-c2ccc(-c3ccc(N(c4cccc(-c5ccc6oc7ccccc7c6c5)c4)c4cccc5oc6c7ccccc7ccc6c45)cc3)cc2)cc1 ; c1ccc(-c2cc(-c3ccccc3)cc(N(c3cccc(-c4ccc5oc6ccccc6c5c4)c3)c3cccc4oc5c6ccccc6ccc5c34)c2)cc1]

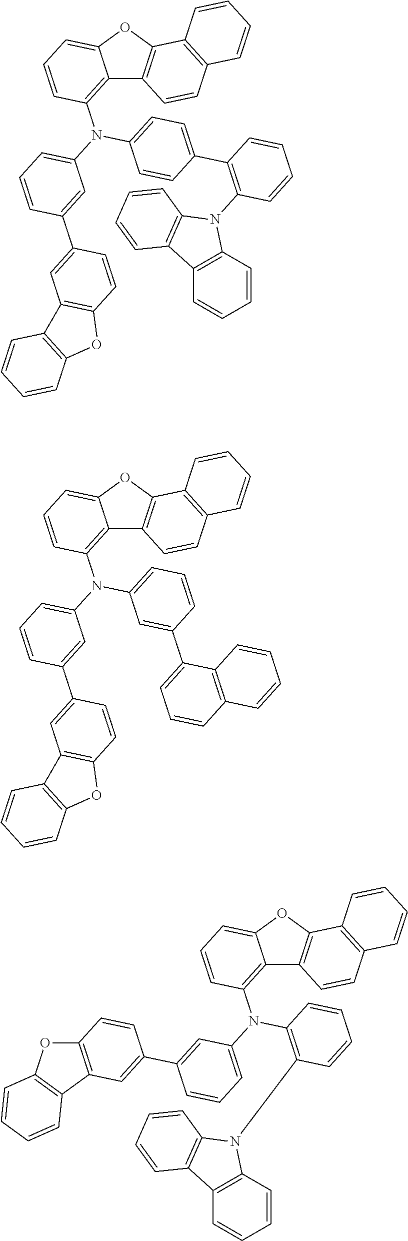 [SMILES: c1cc(-c2ccc3oc4ccccc4c3c2)cc(N(c2ccc(-c3ccccc3-n3c4ccccc4c4ccccc43)cc2)c2cccc3oc4c5ccccc5ccc4c23)c1 ; c1cc(-c2ccc3oc4ccccc4c3c2)cc(N(c2cccc(-c3cccc4ccccc34)c2)c2cccc3oc4c5ccccc5ccc4c23)c1 ; c1cc(-c2ccc3oc4ccccc4c3c2)cc(N(c2ccccc2-n2c3ccccc3c3ccccc32)c2cccc3oc4c5ccccc5ccc4c23)c1]

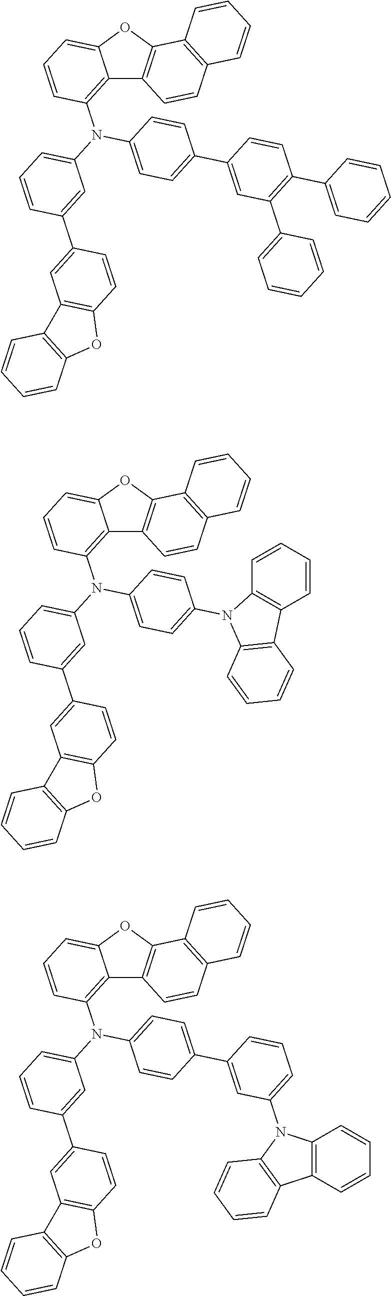 [SMILES: c1ccc(-c2ccc(-c3ccc(N(c4cccc(-c5ccc6oc7ccccc7c6c5)c4)c4cccc5oc6c7ccccc7ccc6c45)cc3)cc2-c2ccccc2)cc1 ; c1cc(-c2ccc3oc4ccccc4c3c2)cc(N(c2ccc(-n3c4ccccc4c4ccccc43)cc2)c2cccc3oc4c5ccccc5ccc4c23)c1 ; c1cc(-c2ccc3oc4ccccc4c3c2)cc(N(c2ccc(-c3cccc(-n4c5ccccc5c5ccccc54)c3)cc2)c2cccc3oc4c5ccccc5ccc4c23)c1]

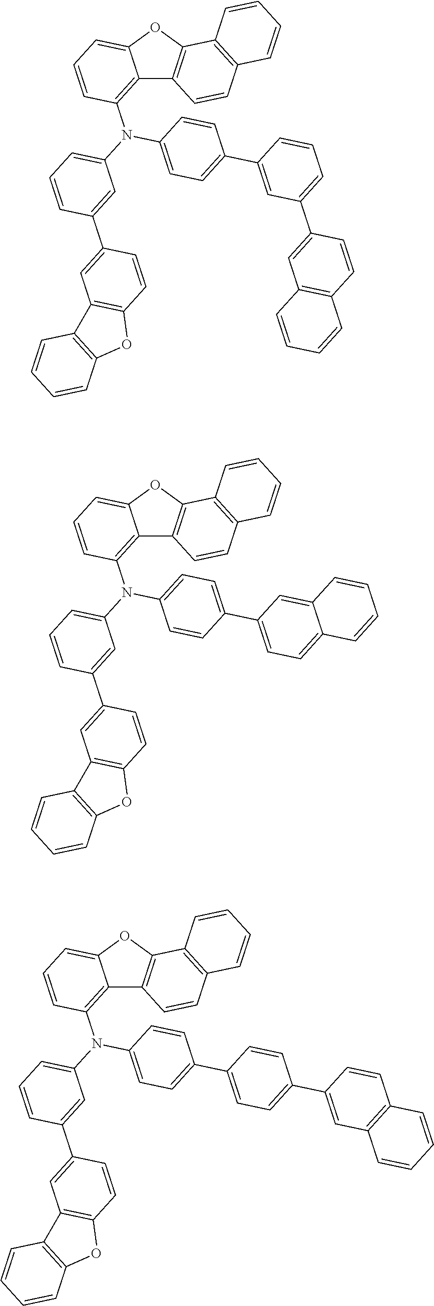 [SMILES: c1cc(-c2ccc(N(c3cccc(-c4ccc5oc6ccccc6c5c4)c3)c3cccc4oc5c6ccccc6ccc5c34)cc2)cc(-c2ccc3ccccc3c2)c1 ; c1cc(-c2ccc3oc4ccccc4c3c2)cc(N(c2ccc(-c3ccc4ccccc4c3)cc2)c2cccc3oc4c5ccccc5ccc4c23)c1 ; c1cc(-c2ccc3oc4ccccc4c3c2)cc(N(c2ccc(-c3ccc(-c4ccc5ccccc5c4)cc3)cc2)c2cccc3oc4c5ccccc5ccc4c23)c1]

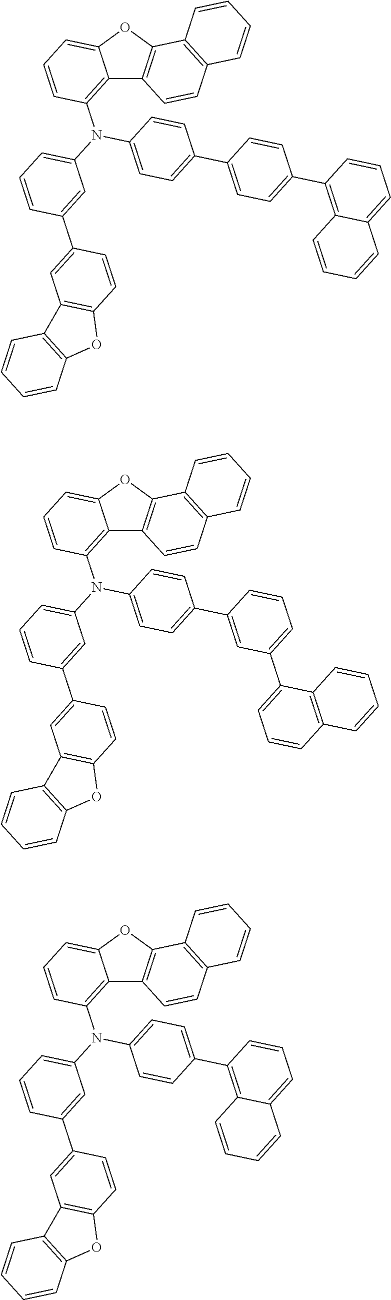 [SMILES: c1cc(-c2ccc3oc4ccccc4c3c2)cc(N(c2ccc(-c3ccc(-c4cccc5ccccc45)cc3)cc2)c2cccc3oc4c5ccccc5ccc4c23)c1 ; c1cc(-c2ccc(N(c3cccc(-c4ccc5oc6ccccc6c5c4)c3)c3cccc4oc5c6ccccc6ccc5c34)cc2)cc(-c2cccc3ccccc23)c1 ; c1cc(-c2ccc3oc4ccccc4c3c2)cc(N(c2ccc(-c3cccc4ccccc34)cc2)c2cccc3oc4c5ccccc5ccc4c23)c1]

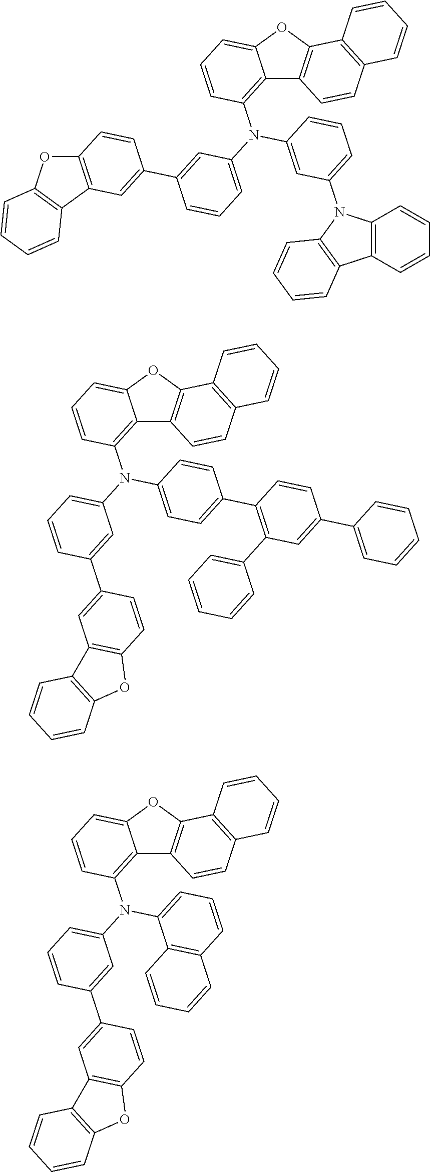 [SMILES: c1cc(-c2ccc3oc4ccccc4c3c2)cc(N(c2cccc(-n3c4ccccc4c4ccccc43)c2)c2cccc3oc4c5ccccc5ccc4c23)c1 ; c1ccc(-c2ccc(-c3ccc(N(c4cccc(-c5ccc6oc7ccccc7c6c5)c4)c4cccc5oc6c7ccccc7ccc6c45)cc3)c(-c3ccccc3)c2)cc1 ; c1cc(-c2ccc3oc4ccccc4c3c2)cc(N(c2cccc3ccccc23)c2cccc3oc4c5ccccc5ccc4c23)c1]

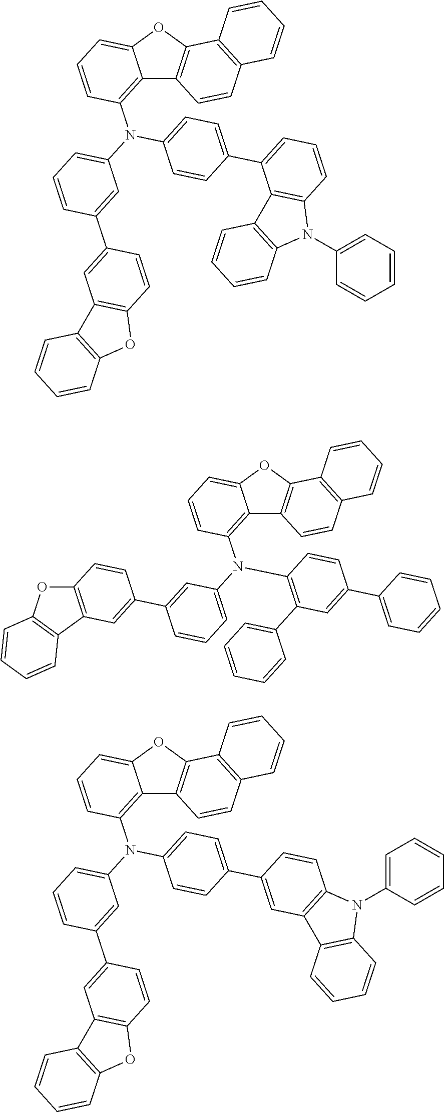

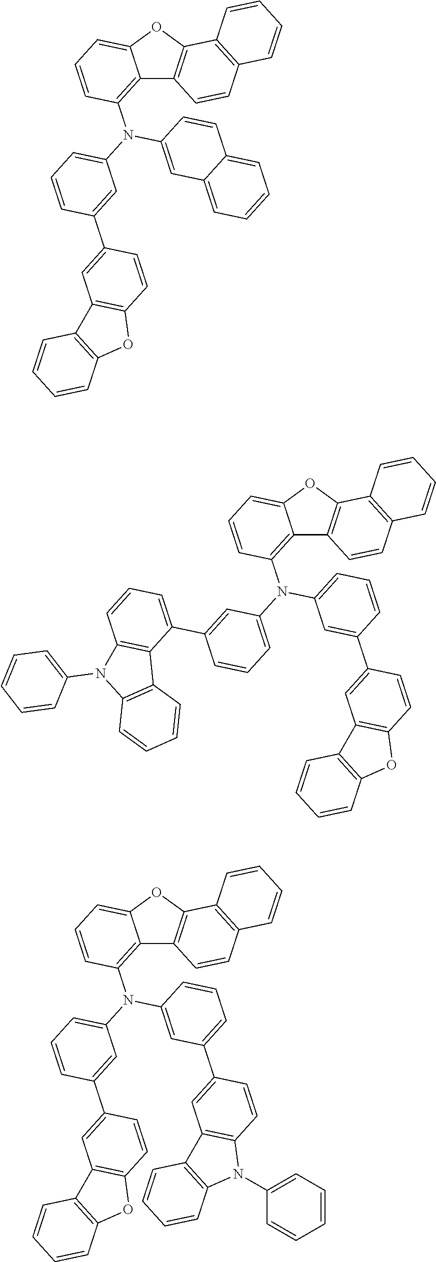 [SMILES: c1cc(-c2ccc3oc4ccccc4c3c2)cc(N(c2ccc3ccccc3c2)c2cccc3oc4c5ccccc5ccc4c23)c1 ; c1ccc(-n2c3ccccc3c3c(-c4cccc(N(c5cccc(-c6ccc7oc8ccccc8c7c6)c5)c5cccc6oc7c8ccccc8ccc7c56)c4)cccc32)cc1 ; c1ccc(-n2c3ccccc3c3cc(-c4cccc(N(c5cccc(-c6ccc7oc8ccccc8c7c6)c5)c5cccc6oc7c8ccccc8ccc7c56)c4)ccc32)cc1]

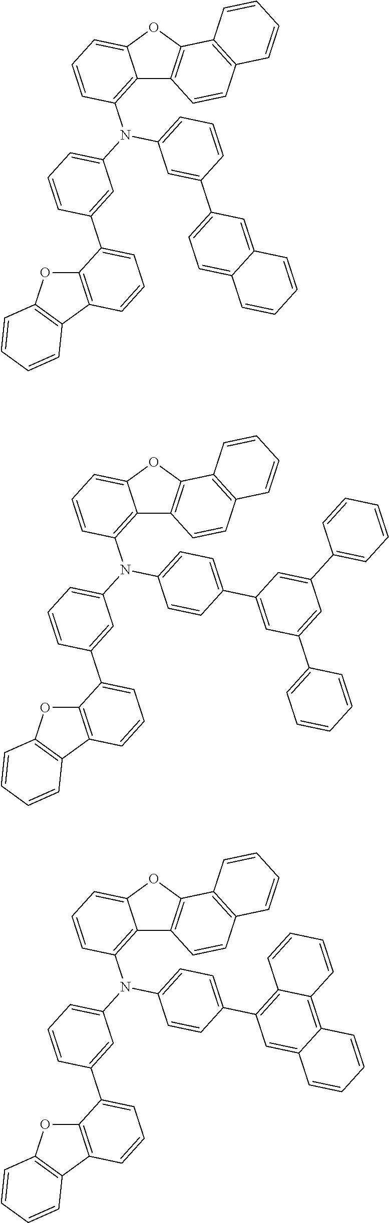 [SMILES: c1cc(-c2ccc3ccccc3c2)cc(N(c2cccc(-c3cccc4c3oc3ccccc34)c2)c2cccc3oc4c5ccccc5ccc4c23)c1 ; c1ccc(-c2cc(-c3ccccc3)cc(-c3ccc(N(c4cccc(-c5cccc6c5oc5ccccc56)c4)c4cccc5oc6c7ccccc7ccc6c45)cc3)c2)cc1 ; c1cc(-c2cccc3c2oc2ccccc23)cc(N(c2ccc(-c3cc4ccccc4c4ccccc34)cc2)c2cccc3oc4c5ccccc5ccc4c23)c1]

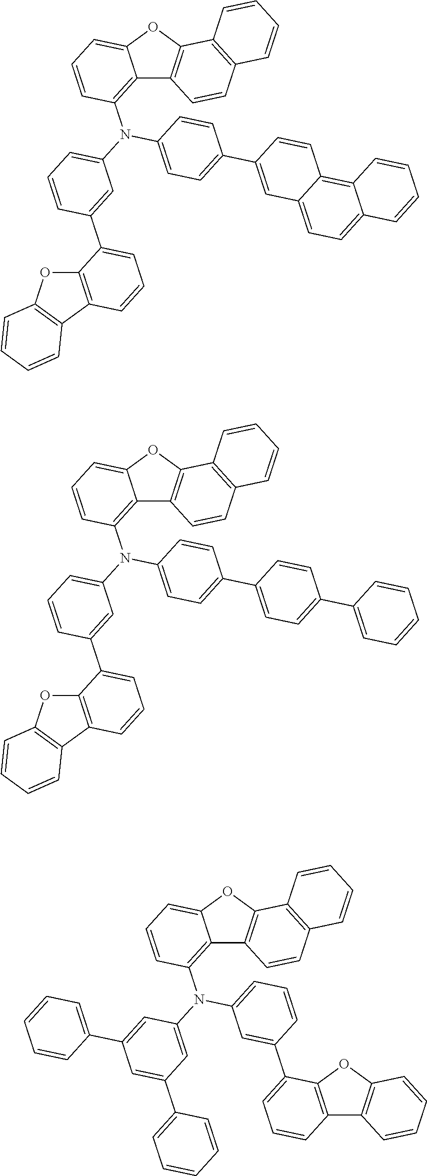 [SMILES: c1cc(-c2cccc3c2oc2ccccc23)cc(N(c2ccc(-c3ccc4c(ccc5ccccc54)c3)cc2)c2cccc3oc4c5ccccc5ccc4c23)c1 ; c1ccc(-c2ccc(-c3ccc(N(c4cccc(-c5cccc6c5oc5ccccc56)c4)c4cccc5oc6c7ccccc7ccc6c45)cc3)cc2)cc1 ; c1ccc(-c2cc(-c3ccccc3)cc(N(c3cccc(-c4cccc5c4oc4ccccc45)c3)c3cccc4oc5c6ccccc6ccc5c34)c2)cc1]

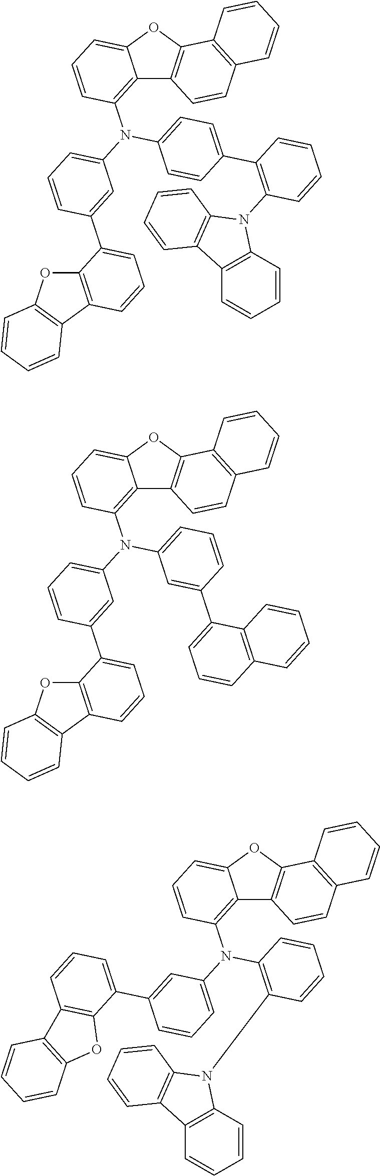 [SMILES: c1cc(-c2cccc3c2oc2ccccc23)cc(N(c2ccc(-c3ccccc3-n3c4ccccc4c4ccccc43)cc2)c2cccc3oc4c5ccccc5ccc4c23)c1 ; c1cc(-c2cccc3ccccc23)cc(N(c2cccc(-c3cccc4c3oc3ccccc34)c2)c2cccc3oc4c5ccccc5ccc4c23)c1 ; c1cc(-c2cccc3c2oc2ccccc23)cc(N(c2ccccc2-n2c3ccccc3c3ccccc32)c2cccc3oc4c5ccccc5ccc4c23)c1]

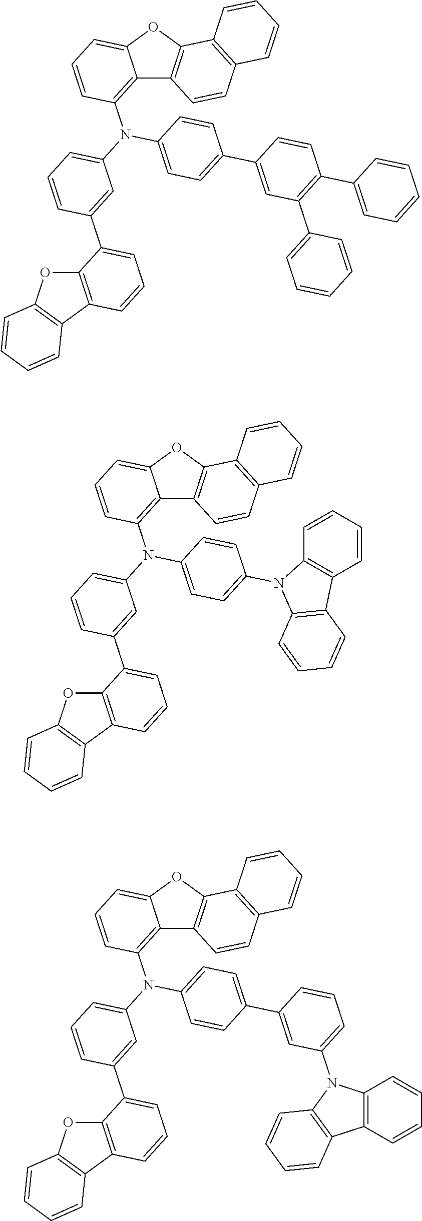 [SMILES: c1ccc(-c2ccc(-c3ccc(N(c4cccc(-c5cccc6c5oc5ccccc56)c4)c4cccc5oc6c7ccccc7ccc6c45)cc3)cc2-c2ccccc2)cc1 ; c1cc(-c2cccc3c2oc2ccccc23)cc(N(c2ccc(-n3c4ccccc4c4ccccc43)cc2)c2cccc3oc4c5ccccc5ccc4c23)c1 ; c1cc(-c2cccc3c2oc2ccccc23)cc(N(c2ccc(-c3cccc(-n4c5ccccc5c5ccccc54)c3)cc2)c2cccc3oc4c5ccccc5ccc4c23)c1]

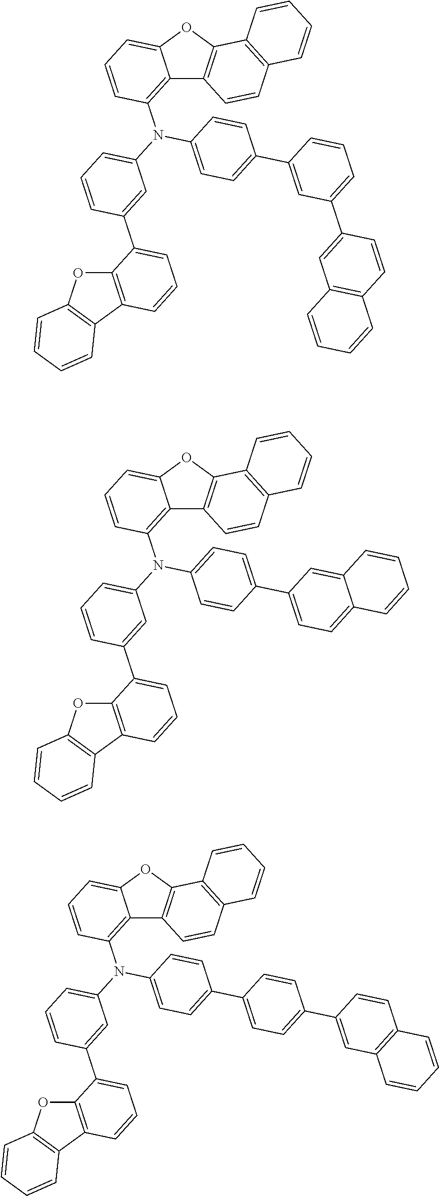 [SMILES: c1cc(-c2ccc(N(c3cccc(-c4cccc5c4oc4ccccc45)c3)c3cccc4oc5c6ccccc6ccc5c34)cc2)cc(-c2ccc3ccccc3c2)c1 ; c1cc(-c2cccc3c2oc2ccccc23)cc(N(c2ccc(-c3ccc4ccccc4c3)cc2)c2cccc3oc4c5ccccc5ccc4c23)c1 ; c1cc(-c2cccc3c2oc2ccccc23)cc(N(c2ccc(-c3ccc(-c4ccc5ccccc5c4)cc3)cc2)c2cccc3oc4c5ccccc5ccc4c23)c1]

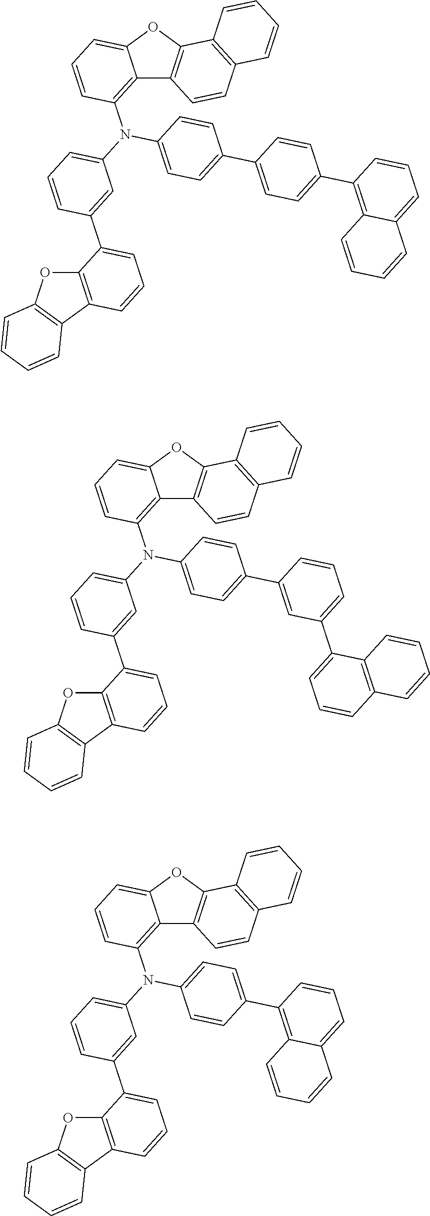 [SMILES: c1cc(-c2cccc3c2oc2ccccc23)cc(N(c2ccc(-c3ccc(-c4cccc5ccccc45)cc3)cc2)c2cccc3oc4c5ccccc5ccc4c23)c1 ; c1cc(-c2ccc(N(c3cccc(-c4cccc5c4oc4ccccc45)c3)c3cccc4oc5c6ccccc6ccc5c34)cc2)cc(-c2cccc3ccccc23)c1 ; c1cc(-c2cccc3c2oc2ccccc23)cc(N(c2ccc(-c3cccc4ccccc34)cc2)c2cccc3oc4c5ccccc5ccc4c23)c1]

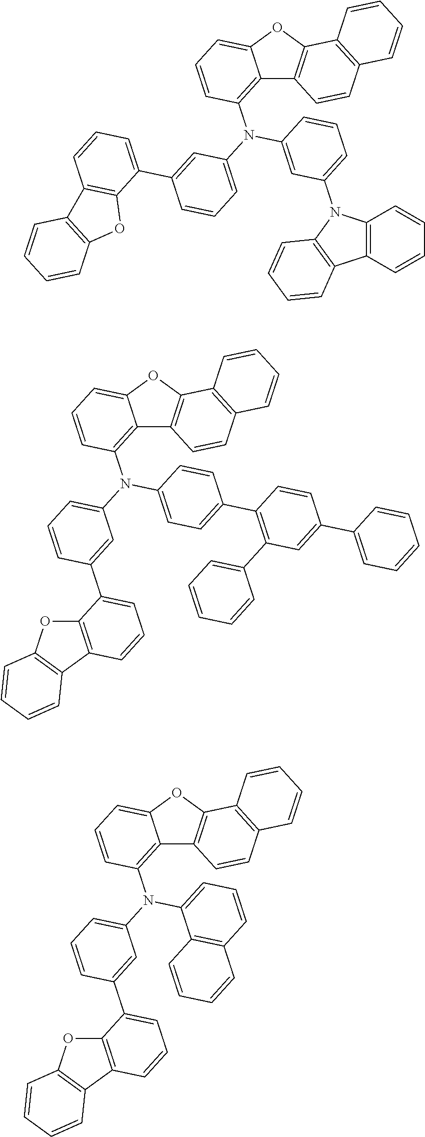 [SMILES: c1cc(-c2cccc3c2oc2ccccc23)cc(N(c2cccc(-n3c4ccccc4c4ccccc43)c2)c2cccc3oc4c5ccccc5ccc4c23)c1 ; c1ccc(-c2ccc(-c3ccc(N(c4cccc(-c5cccc6c5oc5ccccc56)c4)c4cccc5oc6c7ccccc7ccc6c45)cc3)c(-c3ccccc3)c2)cc1 ; c1cc(-c2cccc3c2oc2ccccc23)cc(N(c2cccc3ccccc23)c2cccc3oc4c5ccccc5ccc4c23)c1]

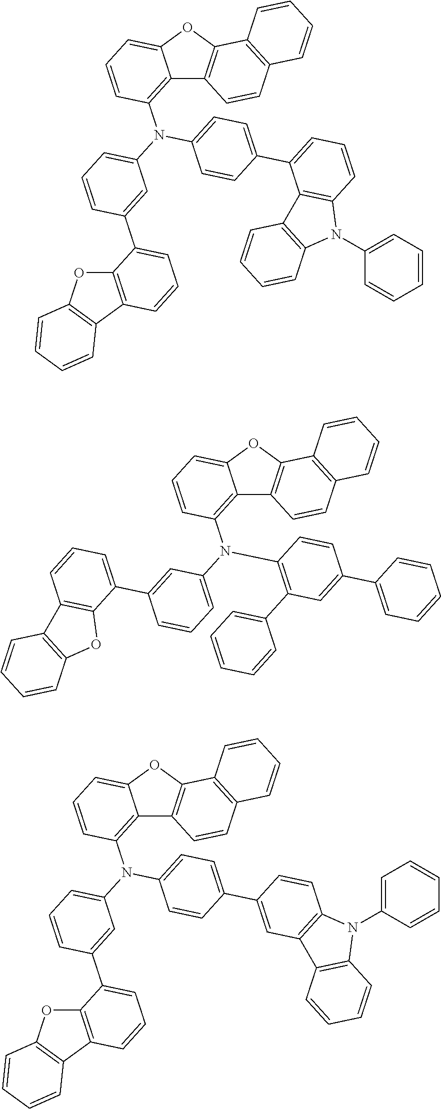 [SMILES: c1ccc(-n2c3ccccc3c3c(-c4ccc(N(c5cccc(-c6cccc7c6oc6ccccc67)c5)c5cccc6oc7c8ccccc8ccc7c56)cc4)cccc32)cc1 ; c1ccc(-c2ccc(N(c3cccc(-c4cccc5c4oc4ccccc45)c3)c3cccc4oc5c6ccccc6ccc5c34)c(-c3ccccc3)c2)cc1 ; c1ccc(-n2c3ccccc3c3cc(-c4ccc(N(c5cccc(-c6cccc7c6oc6ccccc67)c5)c5cccc6oc7c8ccccc8ccc7c56)cc4)ccc32)cc1]

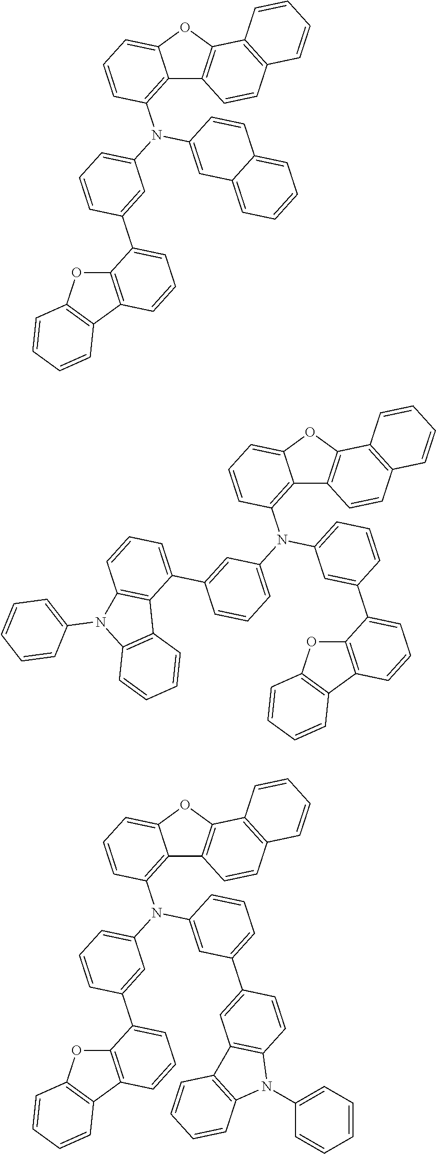

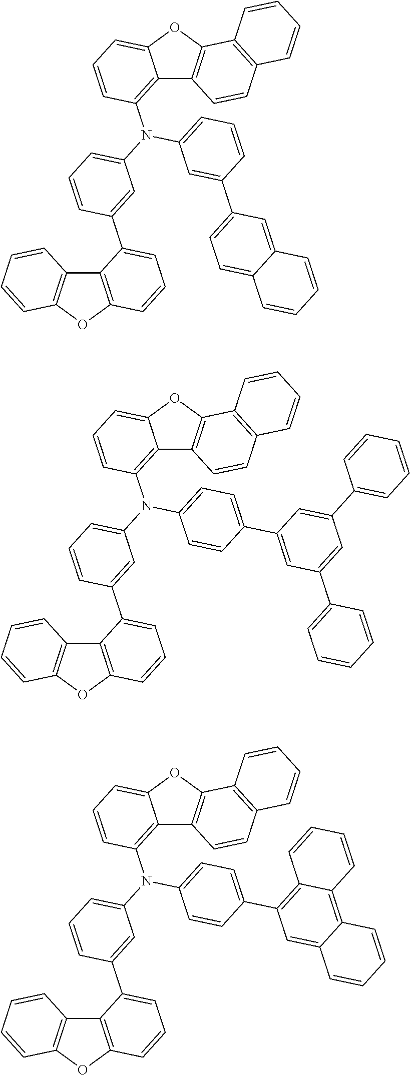 [SMILES: c1cc(-c2ccc3ccccc3c2)cc(N(c2cccc(-c3cccc4oc5ccccc5c34)c2)c2cccc3oc4c5ccccc5ccc4c23)c1 ; c1ccc(-c2cc(-c3ccccc3)cc(-c3ccc(N(c4cccc(-c5cccc6oc7ccccc7c56)c4)c4cccc5oc6c7ccccc7ccc6c45)cc3)c2)cc1 ; c1cc(-c2cccc3oc4ccccc4c23)cc(N(c2ccc(-c3cc4ccccc4c4ccccc34)cc2)c2cccc3oc4c5ccccc5ccc4c23)c1]

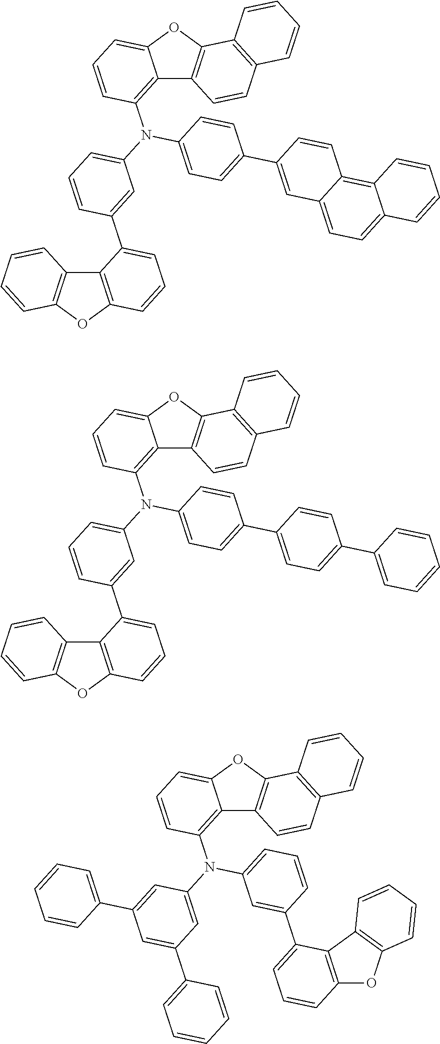 [SMILES: c1cc(-c2cccc3oc4ccccc4c23)cc(N(c2ccc(-c3ccc4c(ccc5ccccc54)c3)cc2)c2cccc3oc4c5ccccc5ccc4c23)c1 ; c1ccc(-c2ccc(-c3ccc(N(c4cccc(-c5cccc6oc7ccccc7c56)c4)c4cccc5oc6c7ccccc7ccc6c45)cc3)cc2)cc1 ; c1ccc(-c2cc(-c3ccccc3)cc(N(c3cccc(-c4cccc5oc6ccccc6c45)c3)c3cccc4oc5c6ccccc6ccc5c34)c2)cc1]

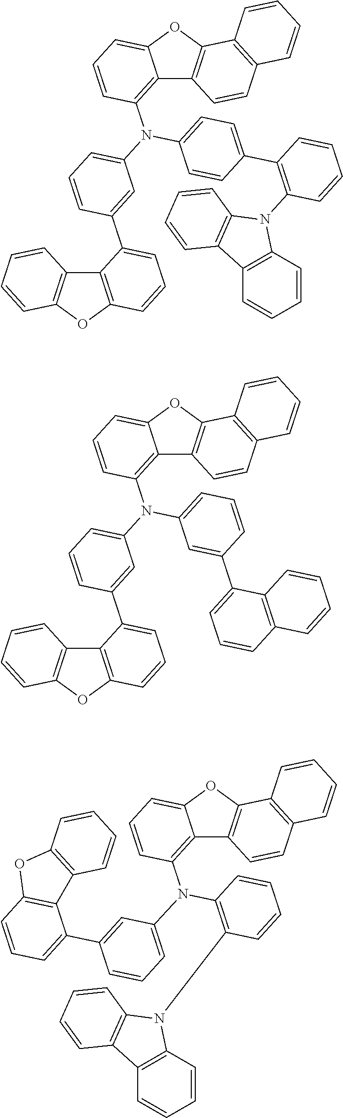 [SMILES: c1cc(-c2cccc3oc4ccccc4c23)cc(N(c2ccc(-c3ccccc3-n3c4ccccc4c4ccccc43)cc2)c2cccc3oc4c5ccccc5ccc4c23)c1 ; c1cc(-c2cccc3ccccc23)cc(N(c2cccc(-c3cccc4oc5ccccc5c34)c2)c2cccc3oc4c5ccccc5ccc4c23)c1 ; c1cc(-c2cccc3oc4ccccc4c23)cc(N(c2ccccc2-n2c3ccccc3c3ccccc32)c2cccc3oc4c5ccccc5ccc4c23)c1]

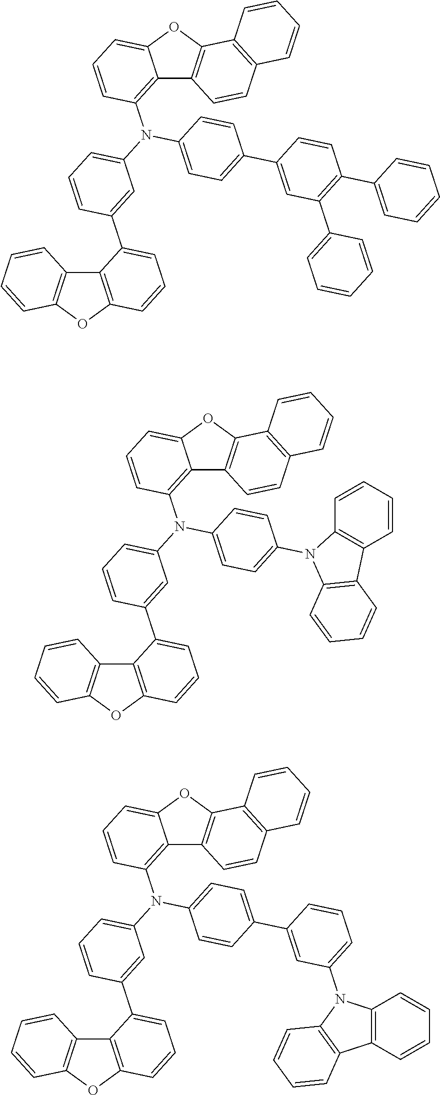 [SMILES: c1ccc(-c2ccc(-c3ccc(N(c4cccc(-c5cccc6oc7ccccc7c56)c4)c4cccc5oc6c7ccccc7ccc6c45)cc3)cc2-c2ccccc2)cc1 ; c1cc(-c2cccc3oc4ccccc4c23)cc(N(c2ccc(-n3c4ccccc4c4ccccc43)cc2)c2cccc3oc4c5ccccc5ccc4c23)c1 ; c1cc(-c2cccc3oc4ccccc4c23)cc(N(c2ccc(-c3cccc(-n4c5ccccc5c5ccccc54)c3)cc2)c2cccc3oc4c5ccccc5ccc4c23)c1]

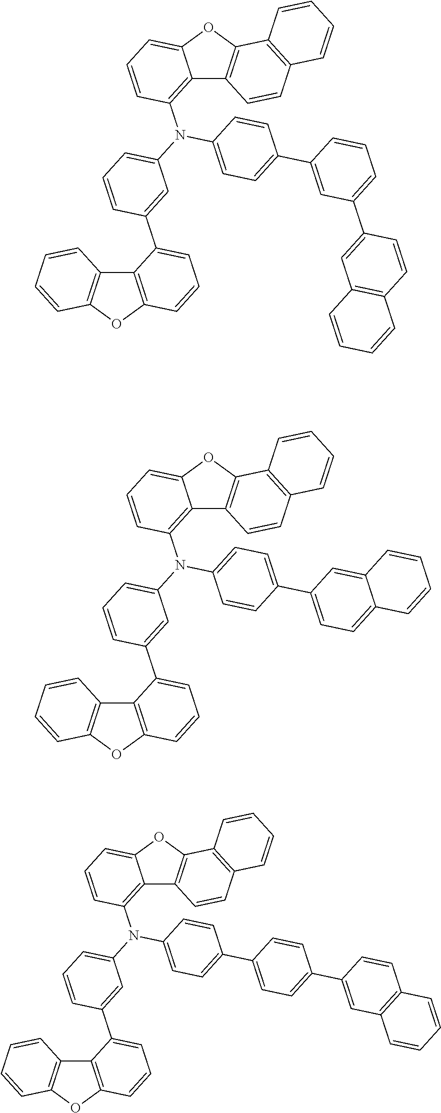 [SMILES: c1cc(-c2ccc(N(c3cccc(-c4cccc5oc6ccccc6c45)c3)c3cccc4oc5c6ccccc6ccc5c34)cc2)cc(-c2ccc3ccccc3c2)c1 ; c1cc(-c2cccc3oc4ccccc4c23)cc(N(c2ccc(-c3ccc4ccccc4c3)cc2)c2cccc3oc4c5ccccc5ccc4c23)c1 ; c1cc(-c2cccc3oc4ccccc4c23)cc(N(c2ccc(-c3ccc(-c4ccc5ccccc5c4)cc3)cc2)c2cccc3oc4c5ccccc5ccc4c23)c1]

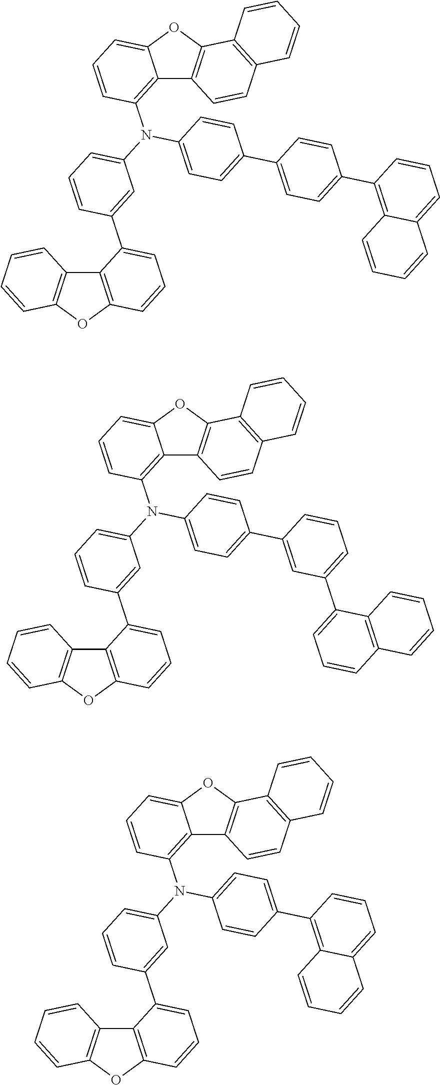 [SMILES: c1cc(-c2cccc3oc4ccccc4c23)cc(N(c2ccc(-c3ccc(-c4cccc5ccccc45)cc3)cc2)c2cccc3oc4c5ccccc5ccc4c23)c1 ; c1cc(-c2ccc(N(c3cccc(-c4cccc5oc6ccccc6c45)c3)c3cccc4oc5c6ccccc6ccc5c34)cc2)cc(-c2cccc3ccccc23)c1 ; c1cc(-c2cccc3oc4ccccc4c23)cc(N(c2ccc(-c3cccc4ccccc34)cc2)c2cccc3oc4c5ccccc5ccc4c23)c1]

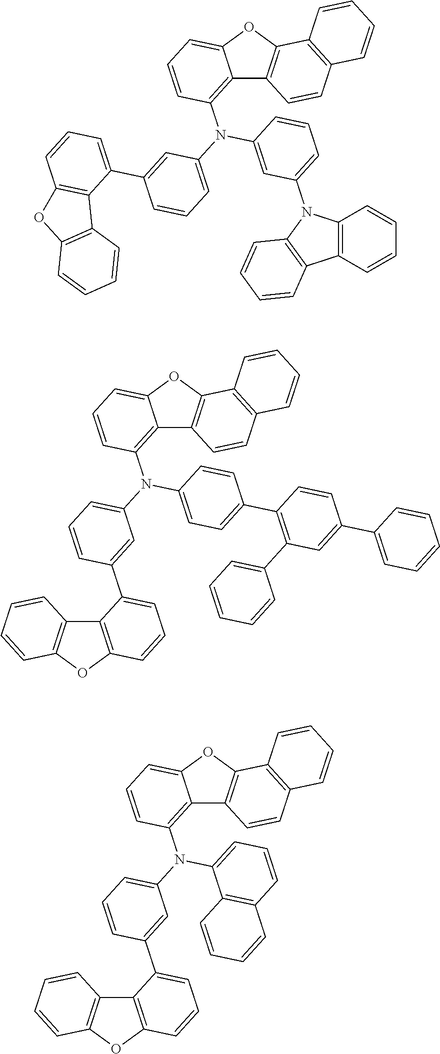 [SMILES: c1cc(-c2cccc3oc4ccccc4c23)cc(N(c2cccc(-n3c4ccccc4c4ccccc43)c2)c2cccc3oc4c5ccccc5ccc4c23)c1 ; c1ccc(-c2ccc(-c3ccc(N(c4cccc(-c5cccc6oc7ccccc7c56)c4)c4cccc5oc6c7ccccc7ccc6c45)cc3)c(-c3ccccc3)c2)cc1 ; c1cc(-c2cccc3oc4ccccc4c23)cc(N(c2cccc3ccccc23)c2cccc3oc4c5ccccc5ccc4c23)c1]

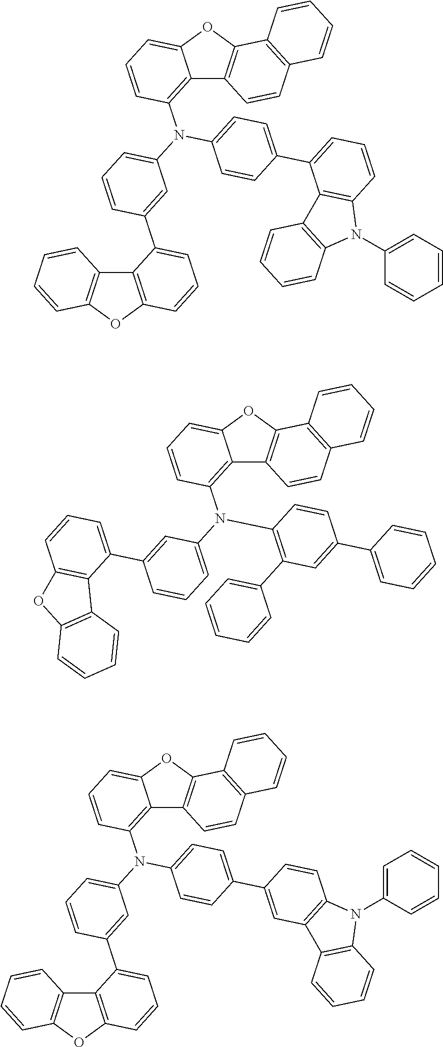 [SMILES: c1ccc(-n2c3ccccc3c3c(-c4ccc(N(c5cccc(-c6cccc7oc8ccccc8c67)c5)c5cccc6oc7c8ccccc8ccc7c56)cc4)cccc32)cc1 ; c1ccc(-c2ccc(N(c3cccc(-c4cccc5oc6ccccc6c45)c3)c3cccc4oc5c6ccccc6ccc5c34)c(-c3ccccc3)c2)cc1 ; c1ccc(-n2c3ccccc3c3cc(-c4ccc(N(c5cccc(-c6cccc7oc8ccccc8c67)c5)c5cccc6oc7c8ccccc8ccc7c56)cc4)ccc32)cc1]

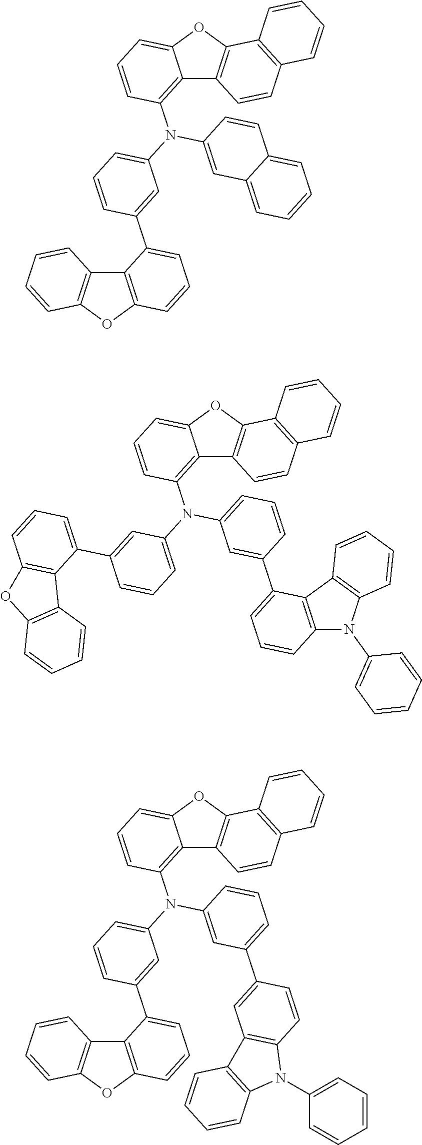 [SMILES: c1cc(-c2cccc3oc4ccccc4c23)cc(N(c2ccc3ccccc3c2)c2cccc3oc4c5ccccc5ccc4c23)c1 ; c1ccc(-n2c3ccccc3c3c(-c4cccc(N(c5cccc(-c6cccc7oc8ccccc8c67)c5)c5cccc6oc7c8ccccc8ccc7c56)c4)cccc32)cc1 ; c1ccc(-n2c3ccccc3c3cc(-c4cccc(N(c5cccc(-c6cccc7oc8ccccc8c67)c5)c5cccc6oc7c8ccccc8ccc7c56)c4)ccc32)cc1]

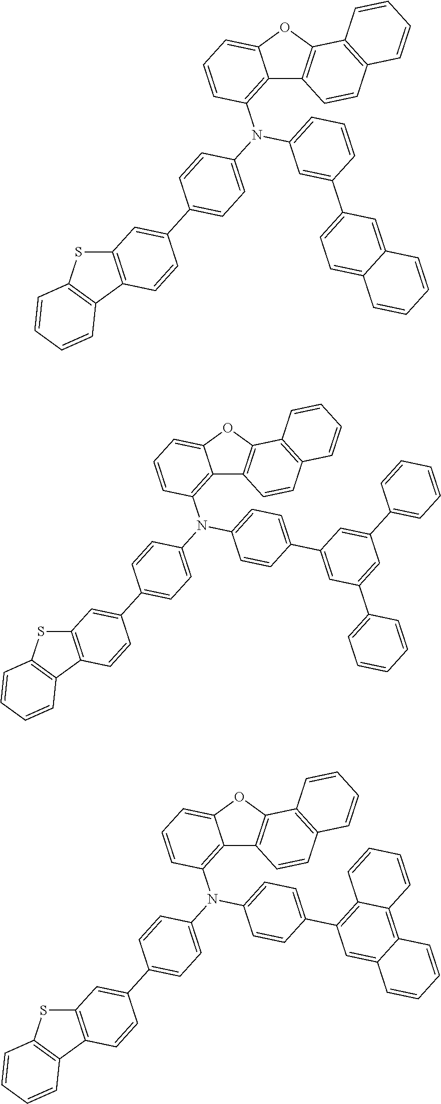 [SMILES: c1cc(-c2ccc3ccccc3c2)cc(N(c2ccc(-c3ccc4c(c3)sc3ccccc34)cc2)c2cccc3oc4c5ccccc5ccc4c23)c1 ; c1ccc(-c2cc(-c3ccccc3)cc(-c3ccc(N(c4ccc(-c5ccc6c(c5)sc5ccccc56)cc4)c4cccc5oc6c7ccccc7ccc6c45)cc3)c2)cc1 ; c1ccc2c(c1)cc(-c1ccc(N(c3ccc(-c4ccc5c(c4)sc4ccccc45)cc3)c3cccc4oc5c6ccccc6ccc5c34)cc1)c1ccccc12]

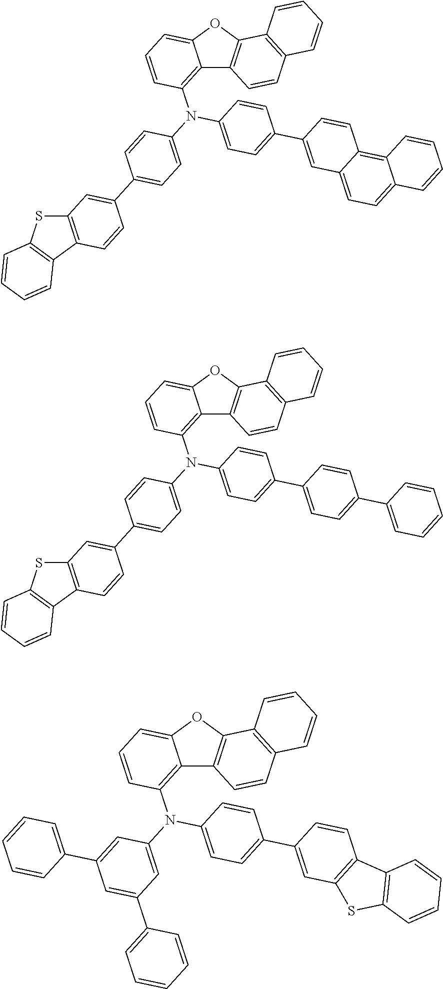 [SMILES: c1ccc2c(c1)ccc1cc(-c3ccc(N(c4ccc(-c5ccc6c(c5)sc5ccccc56)cc4)c4cccc5oc6c7ccccc7ccc6c45)cc3)ccc12 ; c1ccc(-c2ccc(-c3ccc(N(c4ccc(-c5ccc6c(c5)sc5ccccc56)cc4)c4cccc5oc6c7ccccc7ccc6c45)cc3)cc2)cc1 ; c1ccc(-c2cc(-c3ccccc3)cc(N(c3ccc(-c4ccc5c(c4)sc4ccccc45)cc3)c3cccc4oc5c6ccccc6ccc5c34)c2)cc1]

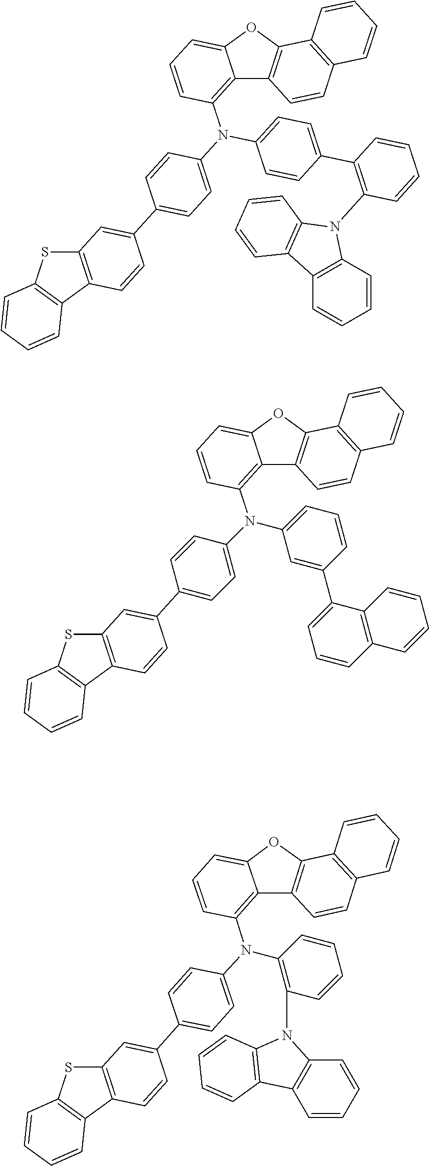 [SMILES: c1ccc(-n2c3ccccc3c3ccccc32)c(-c2ccc(N(c3ccc(-c4ccc5c(c4)sc4ccccc45)cc3)c3cccc4oc5c6ccccc6ccc5c34)cc2)c1 ; c1cc(-c2cccc3ccccc23)cc(N(c2ccc(-c3ccc4c(c3)sc3ccccc34)cc2)c2cccc3oc4c5ccccc5ccc4c23)c1 ; c1ccc(-n2c3ccccc3c3ccccc32)c(N(c2ccc(-c3ccc4c(c3)sc3ccccc34)cc2)c2cccc3oc4c5ccccc5ccc4c23)c1]

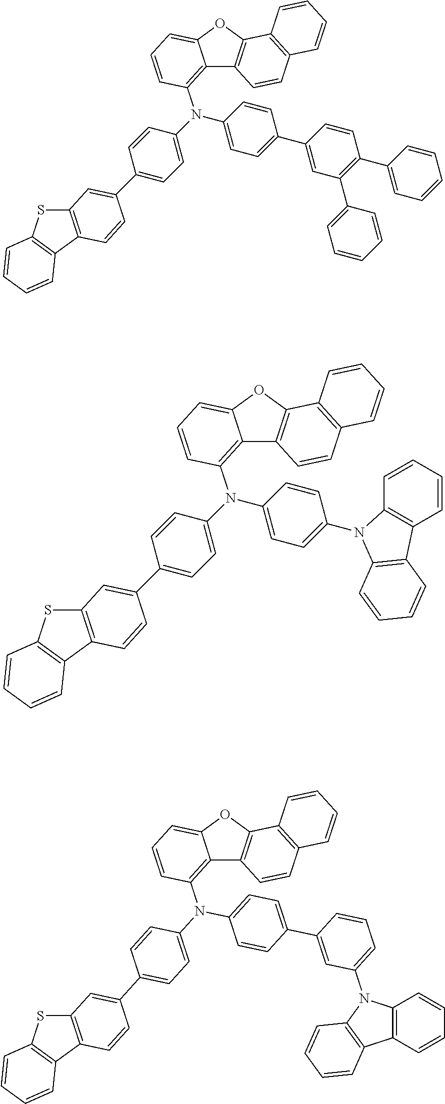 [SMILES: c1ccc(-c2ccc(-c3ccc(N(c4ccc(-c5ccc6c(c5)sc5ccccc56)cc4)c4cccc5oc6c7ccccc7ccc6c45)cc3)cc2-c2ccccc2)cc1 ; c1ccc2c(c1)ccc1c2oc2cccc(N(c3ccc(-c4ccc5c(c4)sc4ccccc45)cc3)c3ccc(-n4c5ccccc5c5ccccc54)cc3)c21 ; c1cc(-c2ccc(N(c3ccc(-c4ccc5c(c4)sc4ccccc45)cc3)c3cccc4oc5c6ccccc6ccc5c34)cc2)cc(-n2c3ccccc3c3ccccc32)c1]

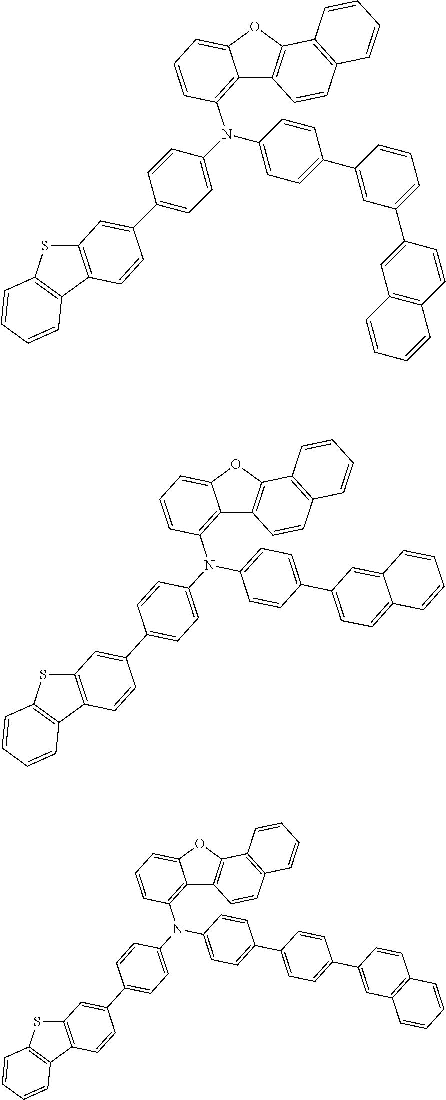 [SMILES: c1cc(-c2ccc(N(c3ccc(-c4ccc5c(c4)sc4ccccc45)cc3)c3cccc4oc5c6ccccc6ccc5c34)cc2)cc(-c2ccc3ccccc3c2)c1 ; c1ccc2cc(-c3ccc(N(c4ccc(-c5ccc6c(c5)sc5ccccc56)cc4)c4cccc5oc6c7ccccc7ccc6c45)cc3)ccc2c1 ; c1ccc2cc(-c3ccc(-c4ccc(N(c5ccc(-c6ccc7c(c6)sc6ccccc67)cc5)c5cccc6oc7c8ccccc8ccc7c56)cc4)cc3)ccc2c1]

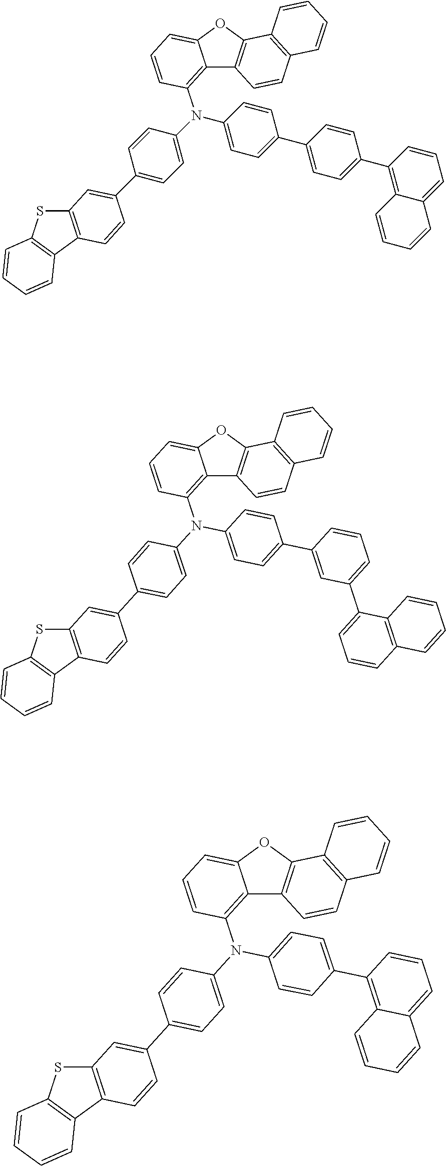 [SMILES: c1ccc2c(-c3ccc(-c4ccc(N(c5ccc(-c6ccc7c(c6)sc6ccccc67)cc5)c5cccc6oc7c8ccccc8ccc7c56)cc4)cc3)cccc2c1 ; c1cc(-c2ccc(N(c3ccc(-c4ccc5c(c4)sc4ccccc45)cc3)c3cccc4oc5c6ccccc6ccc5c34)cc2)cc(-c2cccc3ccccc23)c1 ; c1ccc2c(-c3ccc(N(c4ccc(-c5ccc6c(c5)sc5ccccc56)cc4)c4cccc5oc6c7ccccc7ccc6c45)cc3)cccc2c1]

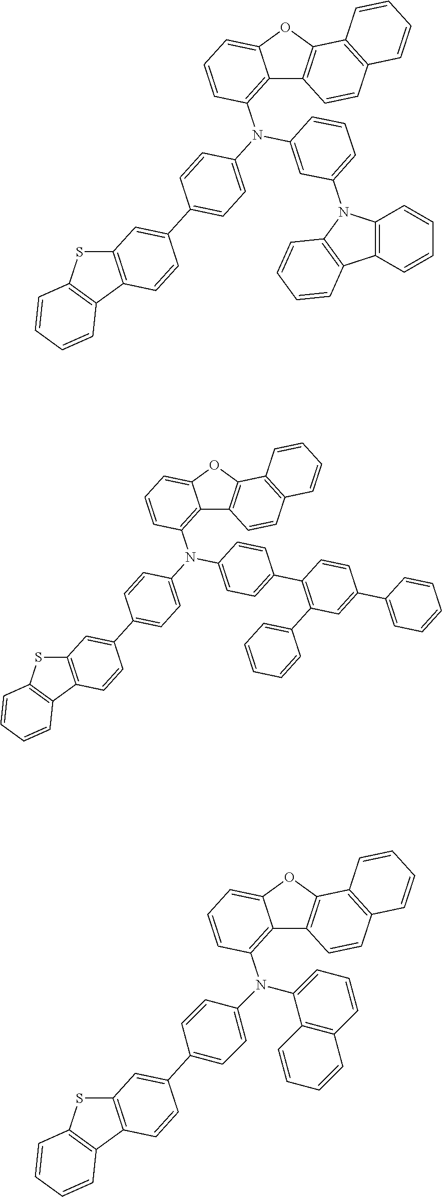 [SMILES: c1cc(N(c2ccc(-c3ccc4c(c3)sc3ccccc34)cc2)c2cccc3oc4c5ccccc5ccc4c23)cc(-n2c3ccccc3c3ccccc32)c1 ; c1ccc(-c2ccc(-c3ccc(N(c4ccc(-c5ccc6c(c5)sc5ccccc56)cc4)c4cccc5oc6c7ccccc7ccc6c45)cc3)c(-c3ccccc3)c2)cc1 ; c1ccc2c(N(c3ccc(-c4ccc5c(c4)sc4ccccc45)cc3)c3cccc4oc5c6ccccc6ccc5c34)cccc2c1]

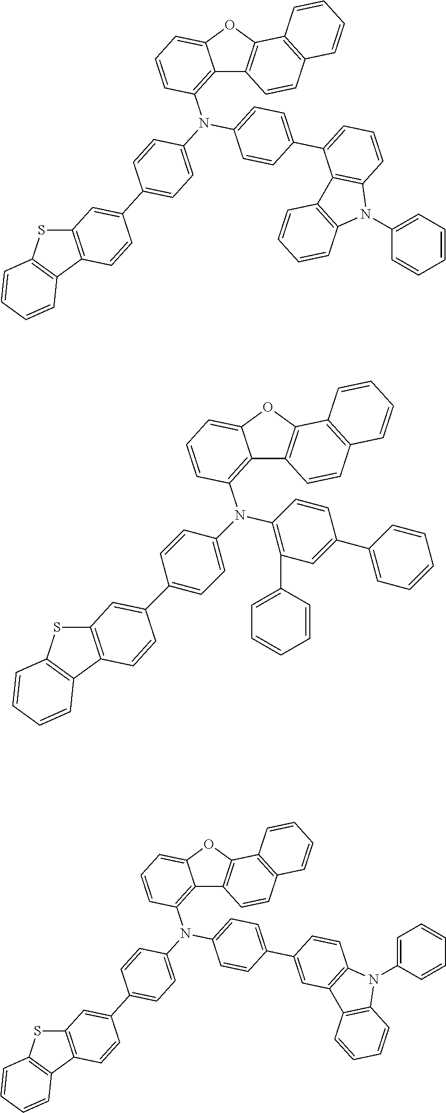 [SMILES: c1ccc(-n2c3ccccc3c3c(-c4ccc(N(c5ccc(-c6ccc7c(c6)sc6ccccc67)cc5)c5cccc6oc7c8ccccc8ccc7c56)cc4)cccc32)cc1 ; c1ccc(-c2ccc(N(c3ccc(-c4ccc5c(c4)sc4ccccc45)cc3)c3cccc4oc5c6ccccc6ccc5c34)c(-c3ccccc3)c2)cc1 ; c1ccc(-n2c3ccccc3c3cc(-c4ccc(N(c5ccc(-c6ccc7c(c6)sc6ccccc67)cc5)c5cccc6oc7c8ccccc8ccc7c56)cc4)ccc32)cc1]

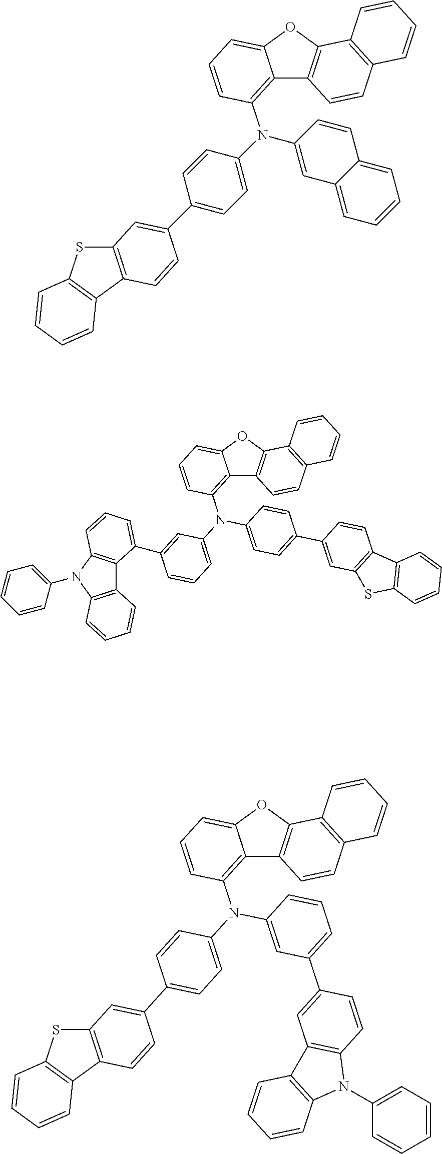 [SMILES: c1ccc2cc(N(c3ccc(-c4ccc5c(c4)sc4ccccc45)cc3)c3cccc4oc5c6ccccc6ccc5c34)ccc2c1 ; c1ccc(-n2c3ccccc3c3c(-c4cccc(N(c5ccc(-c6ccc7c(c6)sc6ccccc67)cc5)c5cccc6oc7c8ccccc8ccc7c56)c4)cccc32)cc1 ; c1ccc(-n2c3ccccc3c3cc(-c4cccc(N(c5ccc(-c6ccc7c(c6)sc6ccccc67)cc5)c5cccc6oc7c8ccccc8ccc7c56)c4)ccc32)cc1]

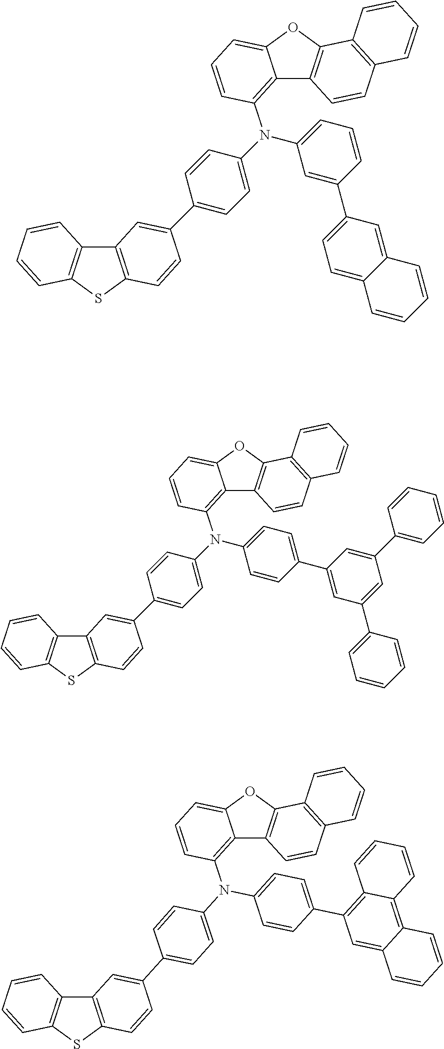 [SMILES: c1cc(-c2ccc3ccccc3c2)cc(N(c2ccc(-c3ccc4sc5ccccc5c4c3)cc2)c2cccc3oc4c5ccccc5ccc4c23)c1 ; c1ccc(-c2cc(-c3ccccc3)cc(-c3ccc(N(c4ccc(-c5ccc6sc7ccccc7c6c5)cc4)c4cccc5oc6c7ccccc7ccc6c45)cc3)c2)cc1 ; c1ccc2c(c1)cc(-c1ccc(N(c3ccc(-c4ccc5sc6ccccc6c5c4)cc3)c3cccc4oc5c6ccccc6ccc5c34)cc1)c1ccccc12]

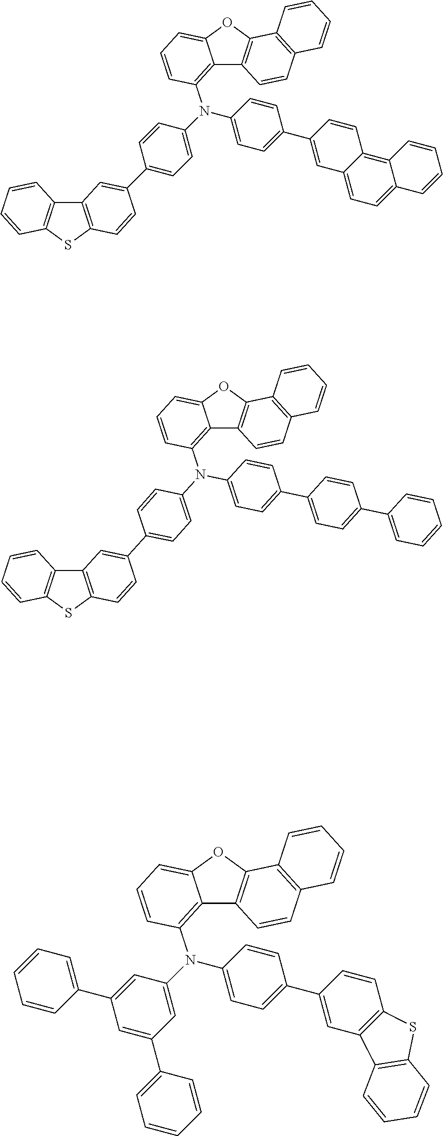 [SMILES: c1ccc2c(c1)ccc1cc(-c3ccc(N(c4ccc(-c5ccc6sc7ccccc7c6c5)cc4)c4cccc5oc6c7ccccc7ccc6c45)cc3)ccc12 ; c1ccc(-c2ccc(-c3ccc(N(c4ccc(-c5ccc6sc7ccccc7c6c5)cc4)c4cccc5oc6c7ccccc7ccc6c45)cc3)cc2)cc1 ; c1ccc(-c2cc(-c3ccccc3)cc(N(c3ccc(-c4ccc5sc6ccccc6c5c4)cc3)c3cccc4oc5c6ccccc6ccc5c34)c2)cc1]

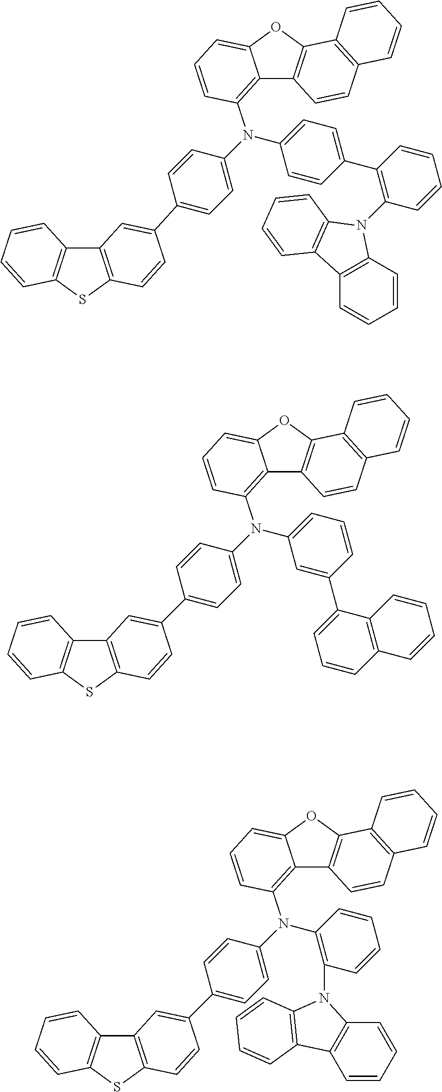 [SMILES: c1ccc(-n2c3ccccc3c3ccccc32)c(-c2ccc(N(c3ccc(-c4ccc5sc6ccccc6c5c4)cc3)c3cccc4oc5c6ccccc6ccc5c34)cc2)c1 ; c1cc(-c2cccc3ccccc23)cc(N(c2ccc(-c3ccc4sc5ccccc5c4c3)cc2)c2cccc3oc4c5ccccc5ccc4c23)c1 ; c1ccc(-n2c3ccccc3c3ccccc32)c(N(c2ccc(-c3ccc4sc5ccccc5c4c3)cc2)c2cccc3oc4c5ccccc5ccc4c23)c1]

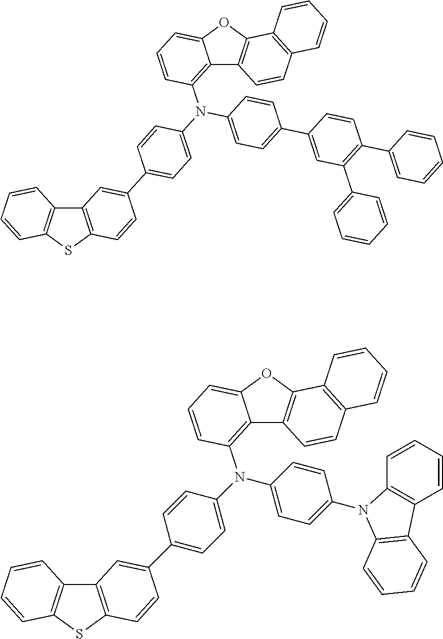 [SMILES: c1ccc(-c2ccc(-c3ccc(N(c4ccc(-c5ccc6sc7ccccc7c6c5)cc4)c4cccc5oc6c7ccccc7ccc6c45)cc3)cc2-c2ccccc2)cc1 ; c1ccc2c(c1)ccc1c2oc2cccc(N(c3ccc(-c4ccc5sc6ccccc6c5c4)cc3)c3ccc(-n4c5ccccc5c5ccccc54)cc3)c21]

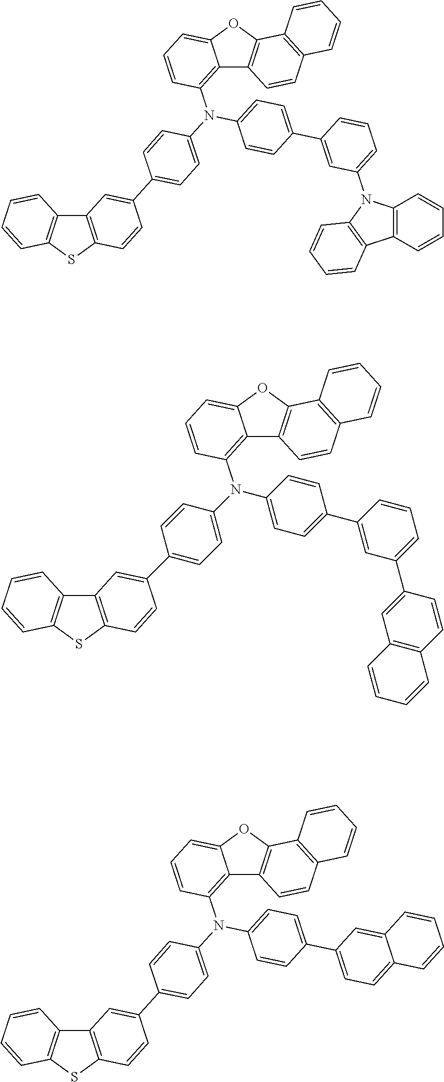 [SMILES: c1cc(-c2ccc(N(c3ccc(-c4ccc5sc6ccccc6c5c4)cc3)c3cccc4oc5c6ccccc6ccc5c34)cc2)cc(-n2c3ccccc3c3ccccc32)c1 ; c1cc(-c2ccc(N(c3ccc(-c4ccc5sc6ccccc6c5c4)cc3)c3cccc4oc5c6ccccc6ccc5c34)cc2)cc(-c2ccc3ccccc3c2)c1 ; c1ccc2cc(-c3ccc(N(c4ccc(-c5ccc6sc7ccccc7c6c5)cc4)c4cccc5oc6c7ccccc7ccc6c45)cc3)ccc2c1]

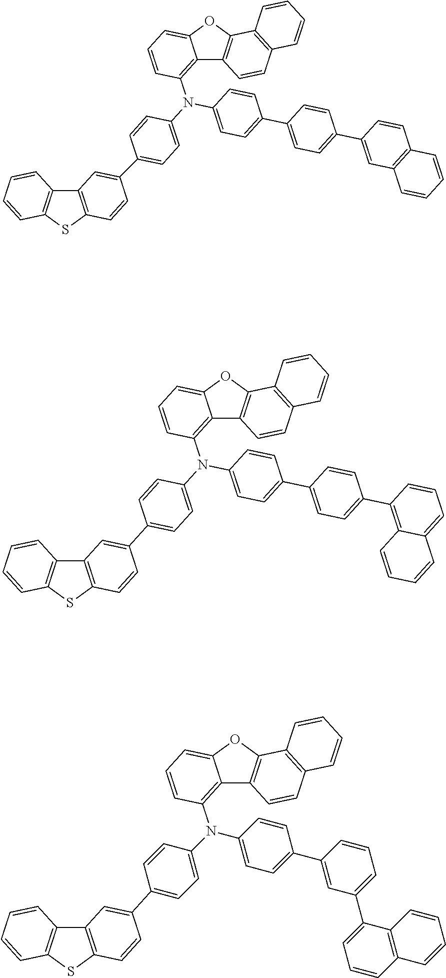 [SMILES: c1ccc2cc(-c3ccc(-c4ccc(N(c5ccc(-c6ccc7sc8ccccc8c7c6)cc5)c5cccc6oc7c8ccccc8ccc7c56)cc4)cc3)ccc2c1 ; c1ccc2c(-c3ccc(-c4ccc(N(c5ccc(-c6ccc7sc8ccccc8c7c6)cc5)c5cccc6oc7c8ccccc8ccc7c56)cc4)cc3)cccc2c1 ; c1cc(-c2ccc(N(c3ccc(-c4ccc5sc6ccccc6c5c4)cc3)c3cccc4oc5c6ccccc6ccc5c34)cc2)cc(-c2cccc3ccccc23)c1]

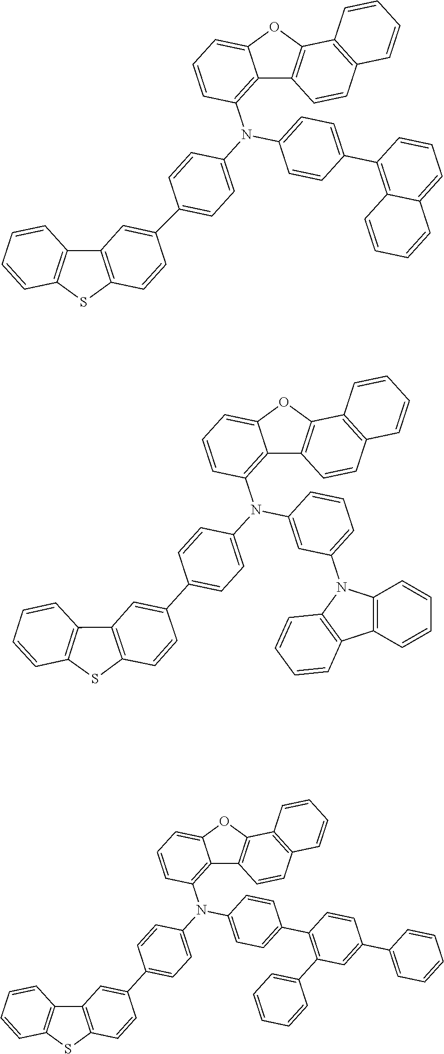 [SMILES: c1ccc2c(-c3ccc(N(c4ccc(-c5ccc6sc7ccccc7c6c5)cc4)c4cccc5oc6c7ccccc7ccc6c45)cc3)cccc2c1 ; c1cc(N(c2ccc(-c3ccc4sc5ccccc5c4c3)cc2)c2cccc3oc4c5ccccc5ccc4c23)cc(-n2c3ccccc3c3ccccc32)c1 ; c1ccc(-c2ccc(-c3ccc(N(c4ccc(-c5ccc6sc7ccccc7c6c5)cc4)c4cccc5oc6c7ccccc7ccc6c45)cc3)c(-c3ccccc3)c2)cc1]

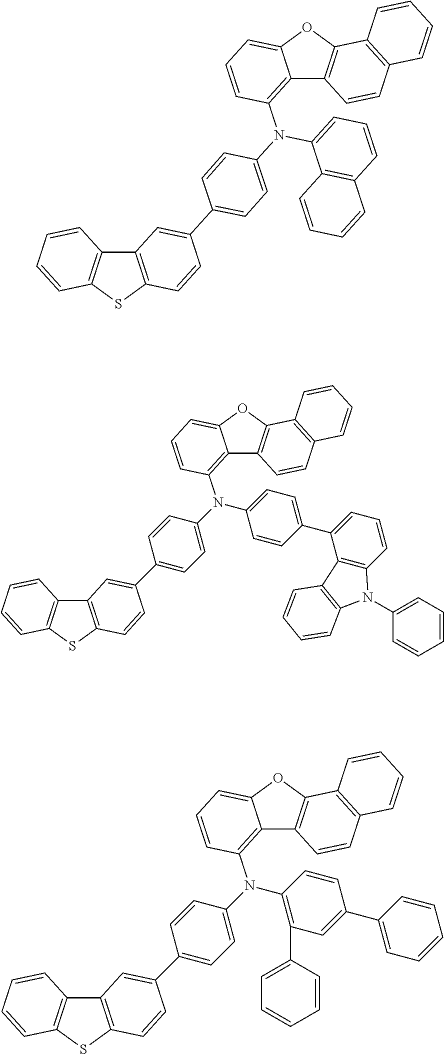 [SMILES: c1ccc2c(N(c3ccc(-c4ccc5sc6ccccc6c5c4)cc3)c3cccc4oc5c6ccccc6ccc5c34)cccc2c1 ; c1ccc(-n2c3ccccc3c3c(-c4ccc(N(c5ccc(-c6ccc7sc8ccccc8c7c6)cc5)c5cccc6oc7c8ccccc8ccc7c56)cc4)cccc32)cc1 ; c1ccc(-c2ccc(N(c3ccc(-c4ccc5sc6ccccc6c5c4)cc3)c3cccc4oc5c6ccccc6ccc5c34)c(-c3ccccc3)c2)cc1]

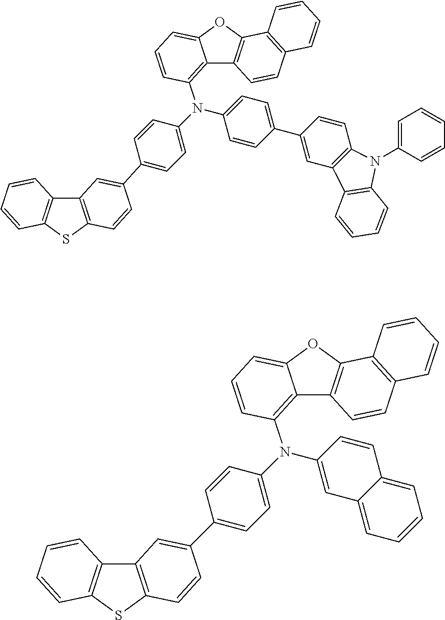 [SMILES: c1ccc(-n2c3ccccc3c3cc(-c4ccc(N(c5ccc(-c6ccc7sc8ccccc8c7c6)cc5)c5cccc6oc7c8ccccc8ccc7c56)cc4)ccc32)cc1 ; c1ccc2cc(N(c3ccc(-c4ccc5sc6ccccc6c5c4)cc3)c3cccc4oc5c6ccccc6ccc5c34)ccc2c1]

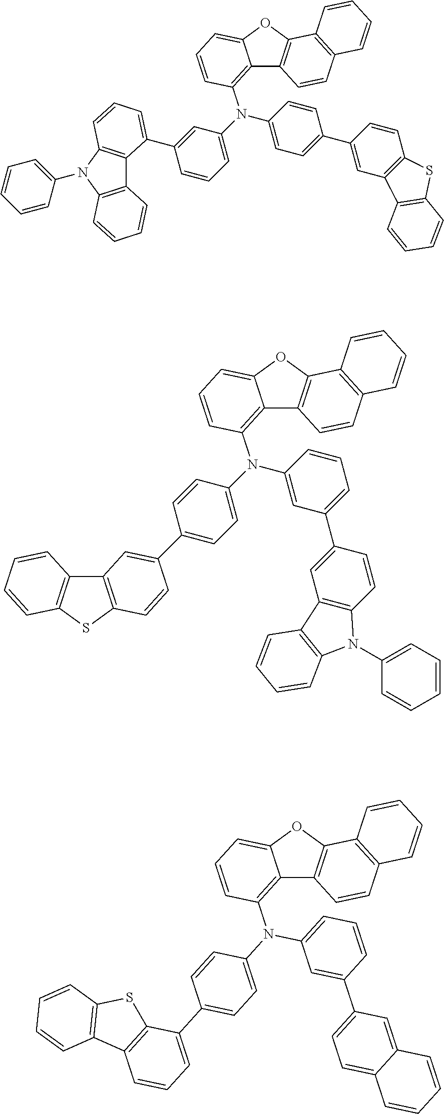 [SMILES: c1ccc(-n2c3ccccc3c3c(-c4cccc(N(c5ccc(-c6ccc7sc8ccccc8c7c6)cc5)c5cccc6oc7c8ccccc8ccc7c56)c4)cccc32)cc1 ; c1ccc(-n2c3ccccc3c3cc(-c4cccc(N(c5ccc(-c6ccc7sc8ccccc8c7c6)cc5)c5cccc6oc7c8ccccc8ccc7c56)c4)ccc32)cc1 ; c1cc(-c2ccc3ccccc3c2)cc(N(c2ccc(-c3cccc4c3sc3ccccc34)cc2)c2cccc3oc4c5ccccc5ccc4c23)c1]

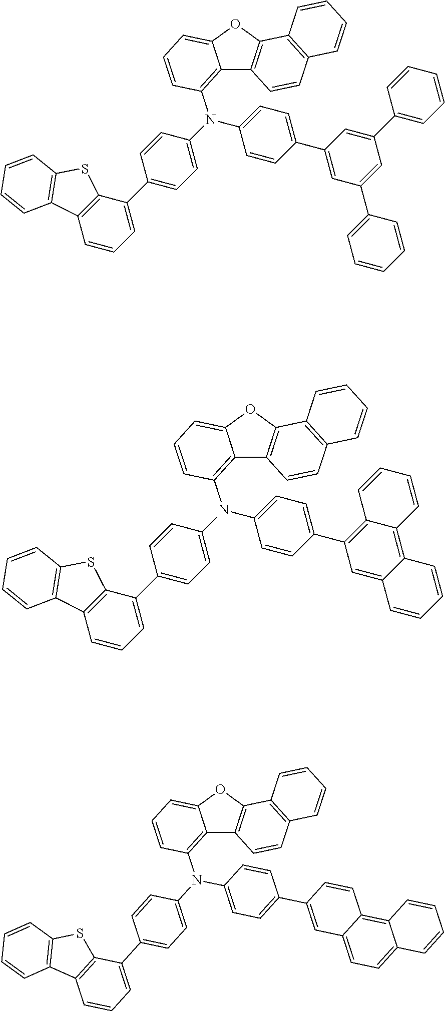 [SMILES: c1ccc(-c2cc(-c3ccccc3)cc(-c3ccc(N(c4ccc(-c5cccc6c5sc5ccccc56)cc4)c4cccc5oc6c7ccccc7ccc6c45)cc3)c2)cc1 ; c1ccc2c(c1)cc(-c1ccc(N(c3ccc(-c4cccc5c4sc4ccccc45)cc3)c3cccc4oc5c6ccccc6ccc5c34)cc1)c1ccccc12 ; c1ccc2c(c1)ccc1cc(-c3ccc(N(c4ccc(-c5cccc6c5sc5ccccc56)cc4)c4cccc5oc6c7ccccc7ccc6c45)cc3)ccc12]

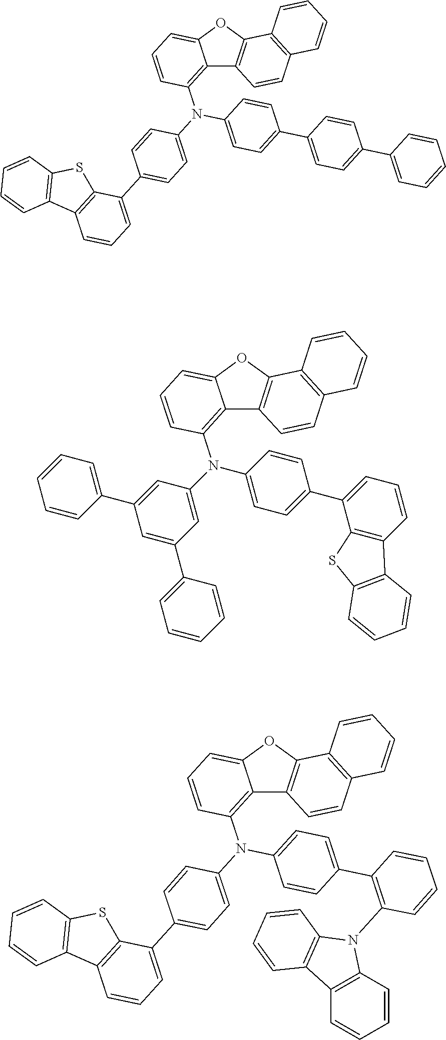 [SMILES: c1ccc(-c2ccc(-c3ccc(N(c4ccc(-c5cccc6c5sc5ccccc56)cc4)c4cccc5oc6c7ccccc7ccc6c45)cc3)cc2)cc1 ; c1ccc(-c2cc(-c3ccccc3)cc(N(c3ccc(-c4cccc5c4sc4ccccc45)cc3)c3cccc4oc5c6ccccc6ccc5c34)c2)cc1 ; c1ccc(-n2c3ccccc3c3ccccc32)c(-c2ccc(N(c3ccc(-c4cccc5c4sc4ccccc45)cc3)c3cccc4oc5c6ccccc6ccc5c34)cc2)c1]

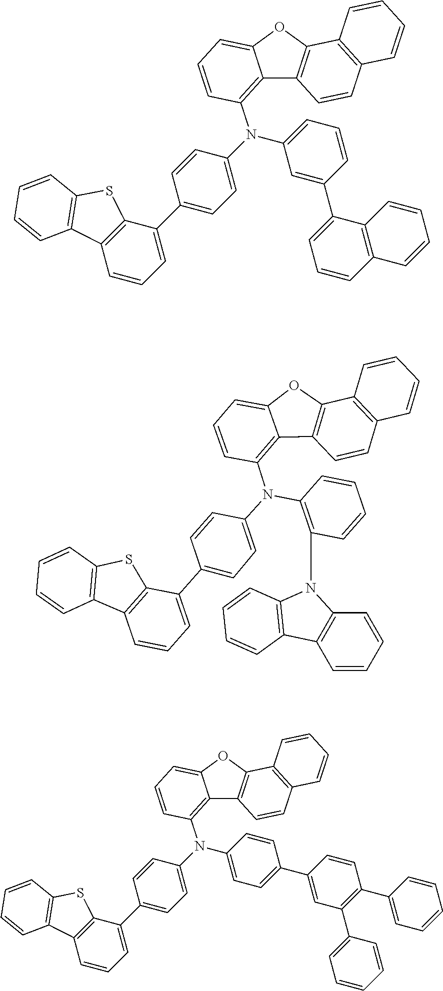 [SMILES: c1cc(-c2cccc3ccccc23)cc(N(c2ccc(-c3cccc4c3sc3ccccc34)cc2)c2cccc3oc4c5ccccc5ccc4c23)c1 ; c1ccc(-n2c3ccccc3c3ccccc32)c(N(c2ccc(-c3cccc4c3sc3ccccc34)cc2)c2cccc3oc4c5ccccc5ccc4c23)c1 ; c1ccc(-c2ccc(-c3ccc(N(c4ccc(-c5cccc6c5sc5ccccc56)cc4)c4cccc5oc6c7ccccc7ccc6c45)cc3)cc2-c2ccccc2)cc1]

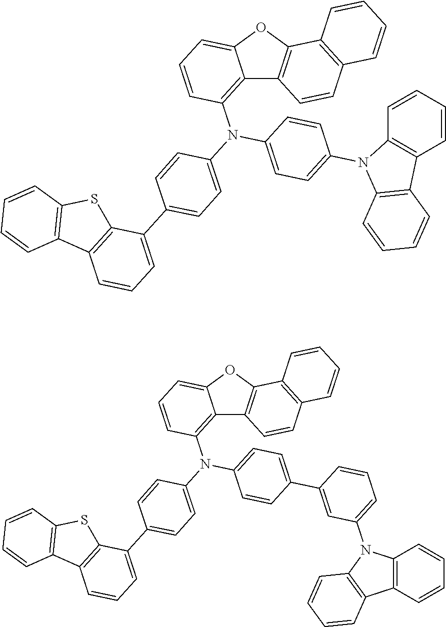 [SMILES: c1ccc2c(c1)ccc1c2oc2cccc(N(c3ccc(-c4cccc5c4sc4ccccc45)cc3)c3ccc(-n4c5ccccc5c5ccccc54)cc3)c21 ; c1cc(-c2ccc(N(c3ccc(-c4cccc5c4sc4ccccc45)cc3)c3cccc4oc5c6ccccc6ccc5c34)cc2)cc(-n2c3ccccc3c3ccccc32)c1]

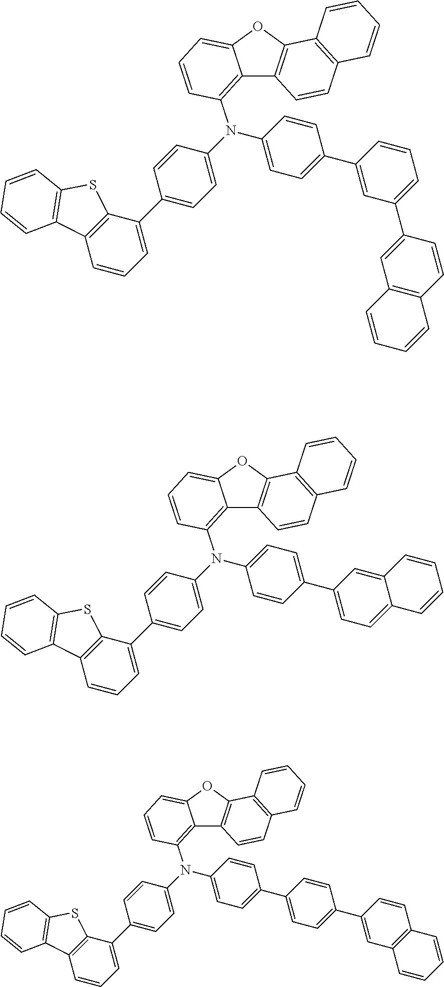 [SMILES: c1cc(-c2ccc(N(c3ccc(-c4cccc5c4sc4ccccc45)cc3)c3cccc4oc5c6ccccc6ccc5c34)cc2)cc(-c2ccc3ccccc3c2)c1 ; c1ccc2cc(-c3ccc(N(c4ccc(-c5cccc6c5sc5ccccc56)cc4)c4cccc5oc6c7ccccc7ccc6c45)cc3)ccc2c1 ; c1ccc2cc(-c3ccc(-c4ccc(N(c5ccc(-c6cccc7c6sc6ccccc67)cc5)c5cccc6oc7c8ccccc8ccc7c56)cc4)cc3)ccc2c1]

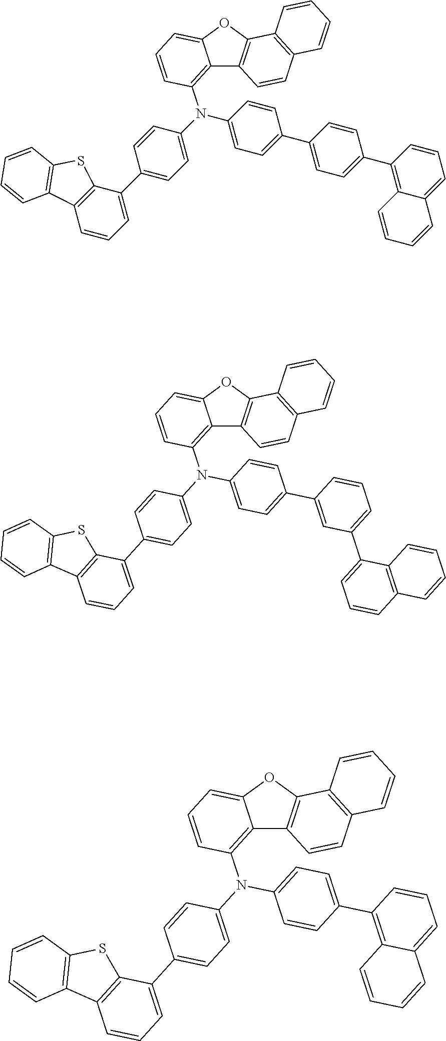 [SMILES: c1ccc2c(-c3ccc(-c4ccc(N(c5ccc(-c6cccc7c6sc6ccccc67)cc5)c5cccc6oc7c8ccccc8ccc7c56)cc4)cc3)cccc2c1 ; c1cc(-c2ccc(N(c3ccc(-c4cccc5c4sc4ccccc45)cc3)c3cccc4oc5c6ccccc6ccc5c34)cc2)cc(-c2cccc3ccccc23)c1 ; c1ccc2c(-c3ccc(N(c4ccc(-c5cccc6c5sc5ccccc56)cc4)c4cccc5oc6c7ccccc7ccc6c45)cc3)cccc2c1]

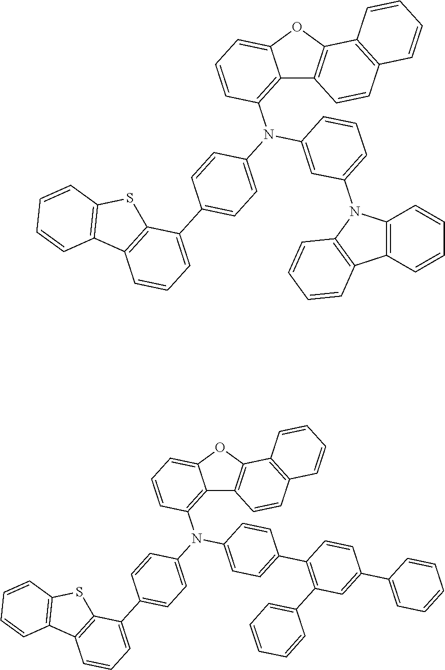 [SMILES: c1cc(N(c2ccc(-c3cccc4c3sc3ccccc34)cc2)c2cccc3oc4c5ccccc5ccc4c23)cc(-n2c3ccccc3c3ccccc32)c1 ; c1ccc(-c2ccc(-c3ccc(N(c4ccc(-c5cccc6c5sc5ccccc56)cc4)c4cccc5oc6c7ccccc7ccc6c45)cc3)c(-c3ccccc3)c2)cc1]

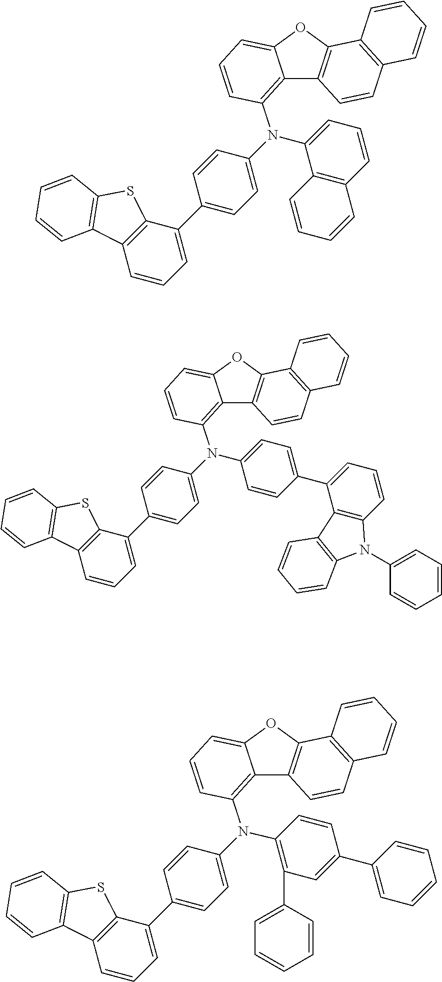 [SMILES: c1ccc2c(N(c3ccc(-c4cccc5c4sc4ccccc45)cc3)c3cccc4oc5c6ccccc6ccc5c34)cccc2c1 ; c1ccc(-n2c3ccccc3c3c(-c4ccc(N(c5ccc(-c6cccc7c6sc6ccccc67)cc5)c5cccc6oc7c8ccccc8ccc7c56)cc4)cccc32)cc1 ; c1ccc(-c2ccc(N(c3ccc(-c4cccc5c4sc4ccccc45)cc3)c3cccc4oc5c6ccccc6ccc5c34)c(-c3ccccc3)c2)cc1]

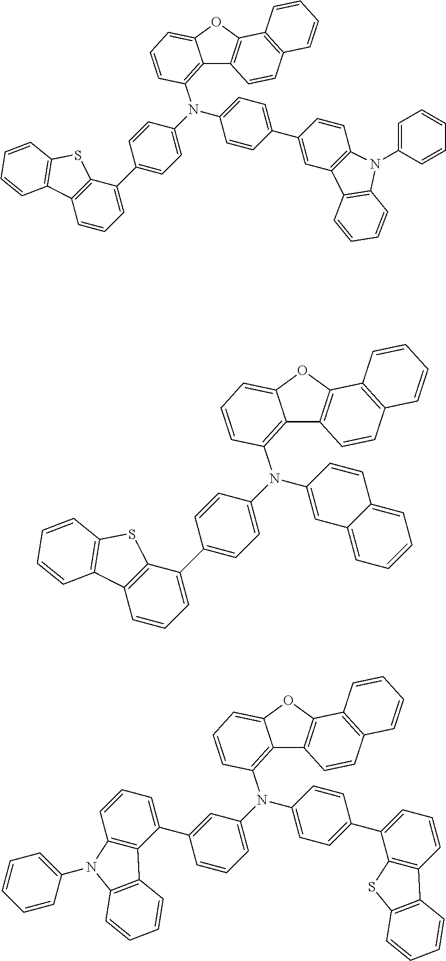 [SMILES: c1ccc(-n2c3ccccc3c3cc(-c4ccc(N(c5ccc(-c6cccc7c6sc6ccccc67)cc5)c5cccc6oc7c8ccccc8ccc7c56)cc4)ccc32)cc1 ; c1ccc2cc(N(c3ccc(-c4cccc5c4sc4ccccc45)cc3)c3cccc4oc5c6ccccc6ccc5c34)ccc2c1 ; c1ccc(-n2c3ccccc3c3c(-c4cccc(N(c5ccc(-c6cccc7c6sc6ccccc67)cc5)c5cccc6oc7c8ccccc8ccc7c56)c4)cccc32)cc1]

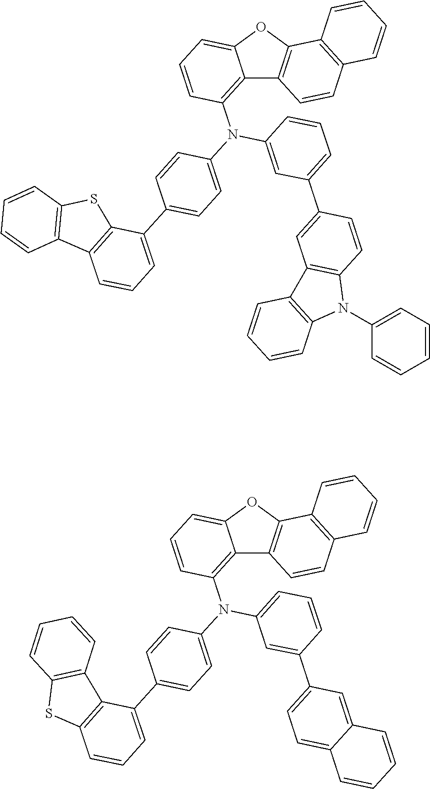 [SMILES: c1ccc(-n2c3ccccc3c3cc(-c4cccc(N(c5ccc(-c6cccc7c6sc6ccccc67)cc5)c5cccc6oc7c8ccccc8ccc7c56)c4)ccc32)cc1 ; c1cc(-c2ccc3ccccc3c2)cc(N(c2ccc(-c3cccc4sc5ccccc5c34)cc2)c2cccc3oc4c5ccccc5ccc4c23)c1]

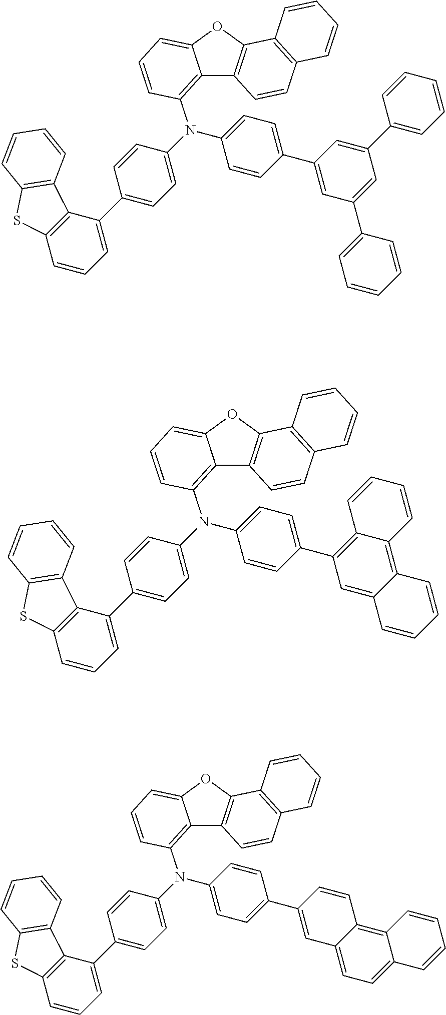 [SMILES: c1ccc(-c2cc(-c3ccccc3)cc(-c3ccc(N(c4ccc(-c5cccc6sc7ccccc7c56)cc4)c4cccc5oc6c7ccccc7ccc6c45)cc3)c2)cc1 ; c1ccc2c(c1)cc(-c1ccc(N(c3ccc(-c4cccc5sc6ccccc6c45)cc3)c3cccc4oc5c6ccccc6ccc5c34)cc1)c1ccccc12 ; c1ccc2c(c1)ccc1cc(-c3ccc(N(c4ccc(-c5cccc6sc7ccccc7c56)cc4)c4cccc5oc6c7ccccc7ccc6c45)cc3)ccc12]

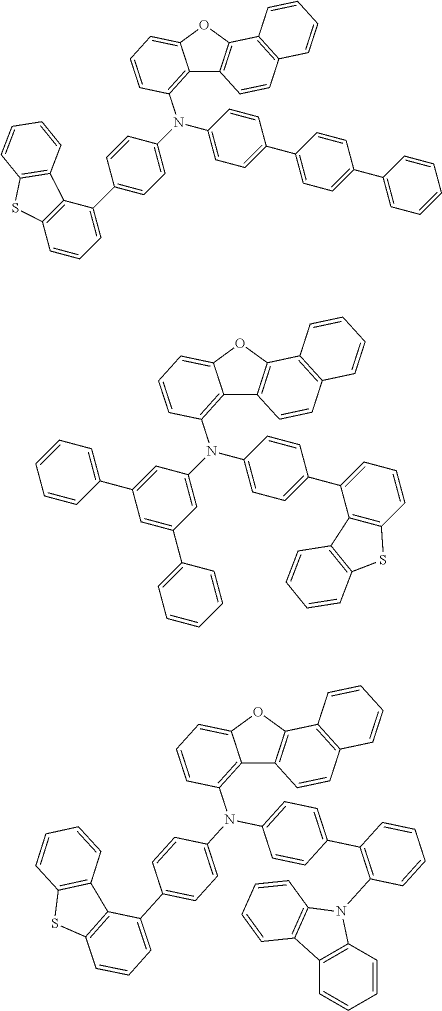 [SMILES: c1ccc(-c2ccc(-c3ccc(N(c4ccc(-c5cccc6sc7ccccc7c56)cc4)c4cccc5oc6c7ccccc7ccc6c45)cc3)cc2)cc1 ; c1ccc(-c2cc(-c3ccccc3)cc(N(c3ccc(-c4cccc5sc6ccccc6c45)cc3)c3cccc4oc5c6ccccc6ccc5c34)c2)cc1 ; c1ccc(-n2c3ccccc3c3ccccc32)c(-c2ccc(N(c3ccc(-c4cccc5sc6ccccc6c45)cc3)c3cccc4oc5c6ccccc6ccc5c34)cc2)c1]

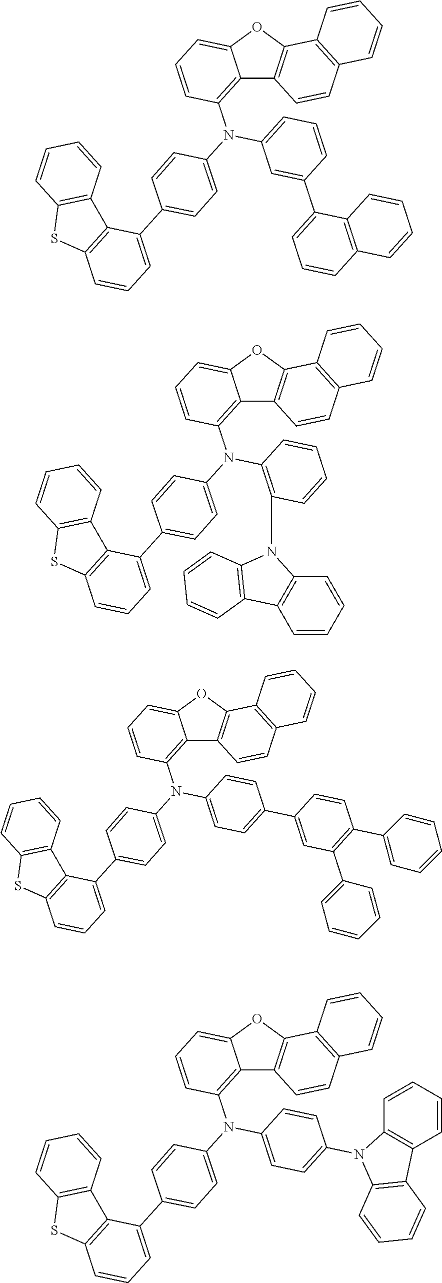

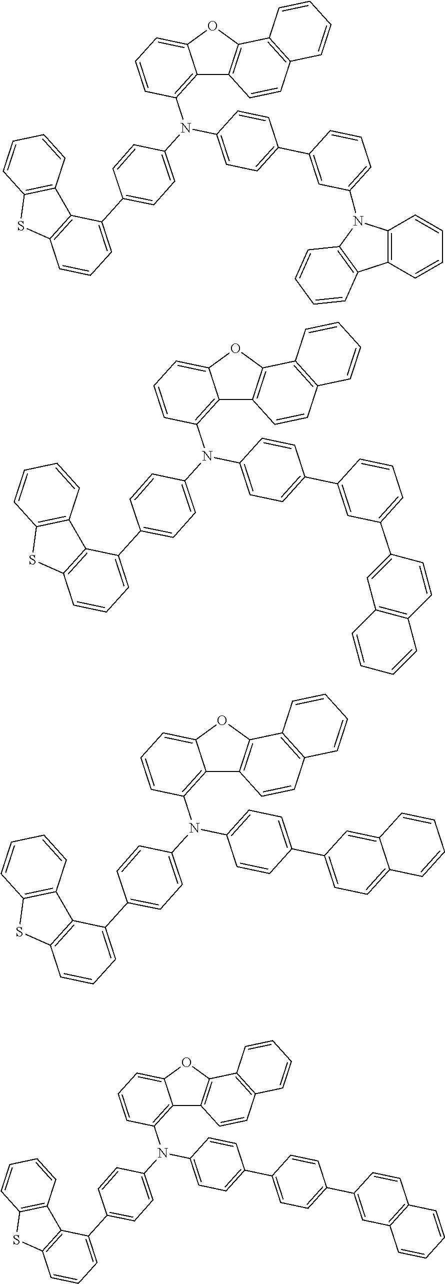

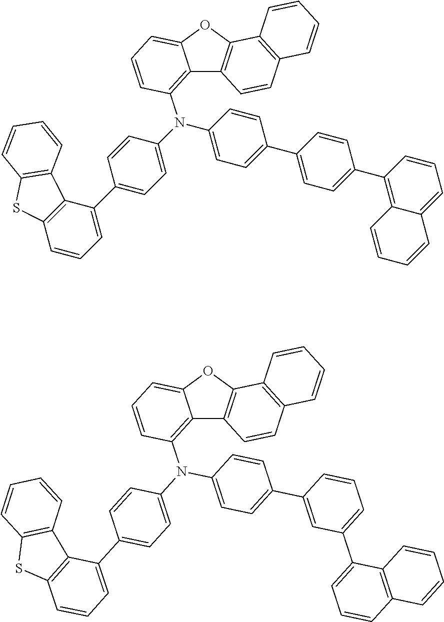 [SMILES: c1ccc2c(-c3ccc(-c4ccc(N(c5ccc(-c6cccc7sc8ccccc8c67)cc5)c5cccc6oc7c8ccccc8ccc7c56)cc4)cc3)cccc2c1 ; c1cc(-c2ccc(N(c3ccc(-c4cccc5sc6ccccc6c45)cc3)c3cccc4oc5c6ccccc6ccc5c34)cc2)cc(-c2cccc3ccccc23)c1]

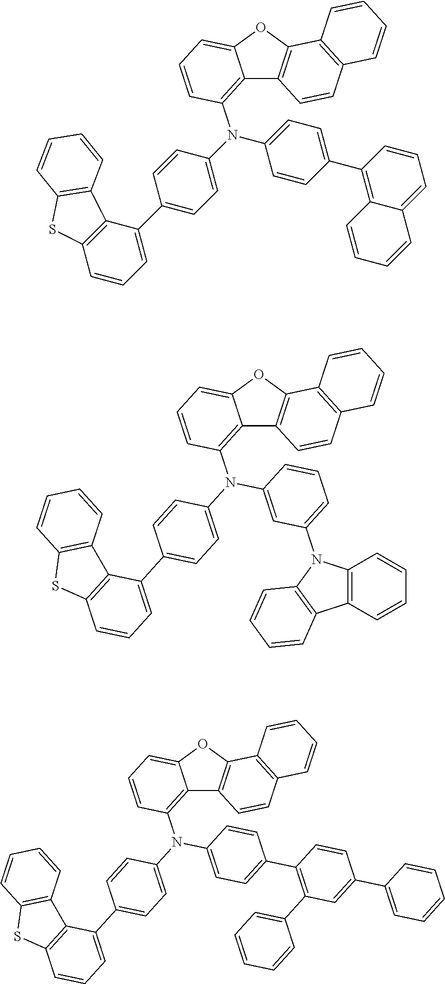 [SMILES: c1ccc2c(-c3ccc(N(c4ccc(-c5cccc6sc7ccccc7c56)cc4)c4cccc5oc6c7ccccc7ccc6c45)cc3)cccc2c1 ; c1cc(N(c2ccc(-c3cccc4sc5ccccc5c34)cc2)c2cccc3oc4c5ccccc5ccc4c23)cc(-n2c3ccccc3c3ccccc32)c1 ; c1ccc(-c2ccc(-c3ccc(N(c4ccc(-c5cccc6sc7ccccc7c56)cc4)c4cccc5oc6c7ccccc7ccc6c45)cc3)c(-c3ccccc3)c2)cc1]

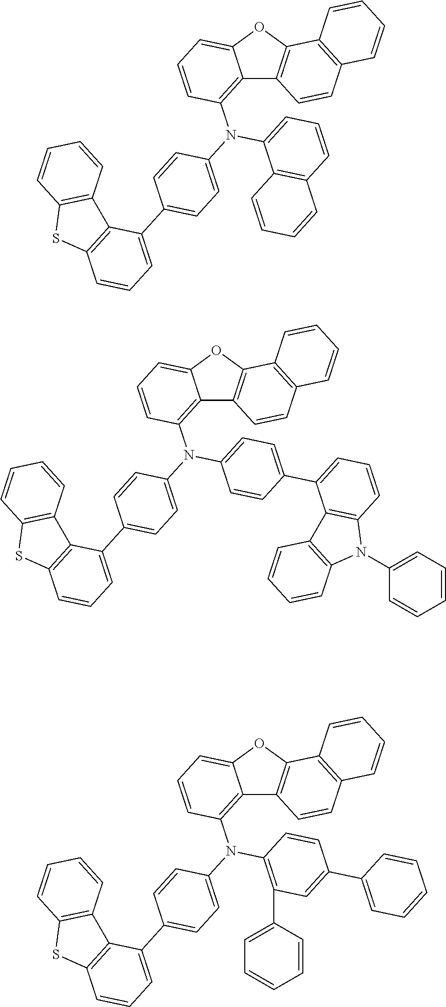 [SMILES: c1ccc2c(N(c3ccc(-c4cccc5sc6ccccc6c45)cc3)c3cccc4oc5c6ccccc6ccc5c34)cccc2c1 ; c1ccc(-n2c3ccccc3c3c(-c4ccc(N(c5ccc(-c6cccc7sc8ccccc8c67)cc5)c5cccc6oc7c8ccccc8ccc7c56)cc4)cccc32)cc1 ; c1ccc(-c2ccc(N(c3ccc(-c4cccc5sc6ccccc6c45)cc3)c3cccc4oc5c6ccccc6ccc5c34)c(-c3ccccc3)c2)cc1]

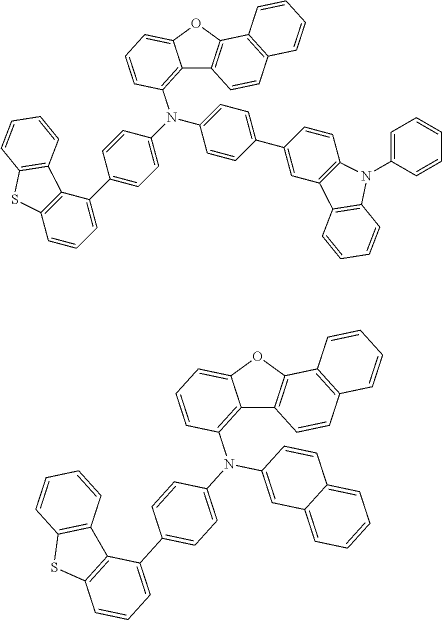 [SMILES: c1ccc(-n2c3ccccc3c3cc(-c4ccc(N(c5ccc(-c6cccc7sc8ccccc8c67)cc5)c5cccc6oc7c8ccccc8ccc7c56)cc4)ccc32)cc1 ; c1ccc2cc(N(c3ccc(-c4cccc5sc6ccccc6c45)cc3)c3cccc4oc5c6ccccc6ccc5c34)ccc2c1]

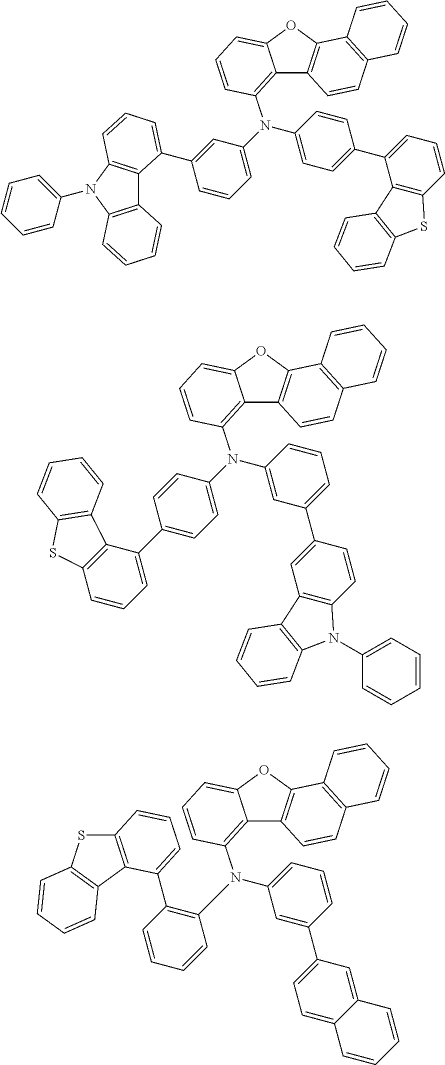

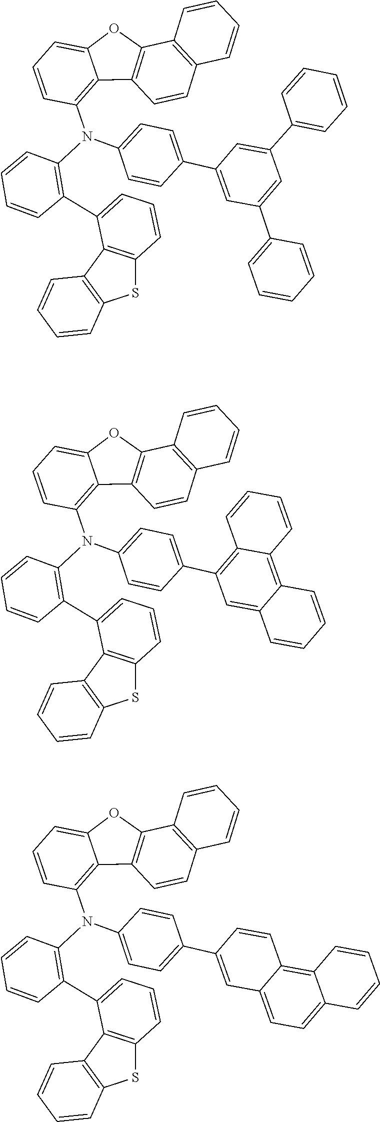 [SMILES: c1ccc(-c2cc(-c3ccccc3)cc(-c3ccc(N(c4ccccc4-c4cccc5sc6ccccc6c45)c4cccc5oc6c7ccccc7ccc6c45)cc3)c2)cc1 ; c1ccc(N(c2ccc(-c3cc4ccccc4c4ccccc34)cc2)c2cccc3oc4c5ccccc5ccc4c23)c(-c2cccc3sc4ccccc4c23)c1 ; c1ccc(N(c2ccc(-c3ccc4c(ccc5ccccc54)c3)cc2)c2cccc3oc4c5ccccc5ccc4c23)c(-c2cccc3sc4ccccc4c23)c1]

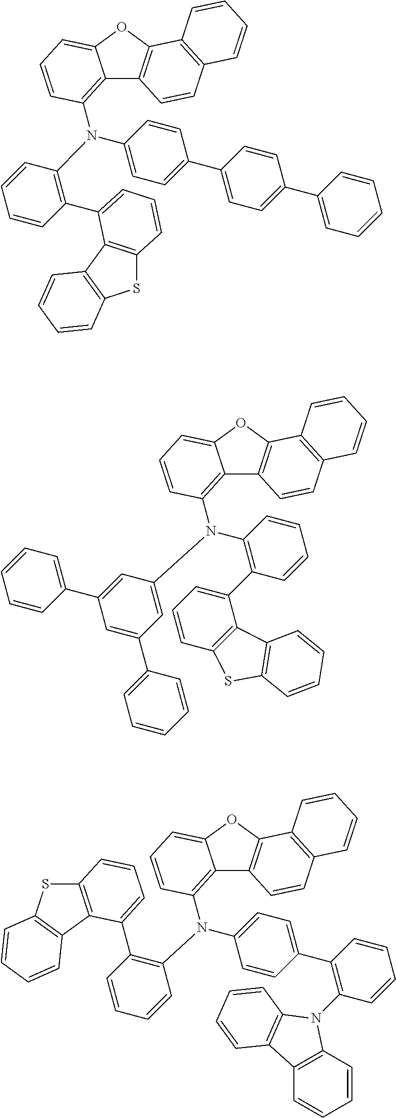 [SMILES: c1ccc(-c2ccc(-c3ccc(N(c4ccccc4-c4cccc5sc6ccccc6c45)c4cccc5oc6c7ccccc7ccc6c45)cc3)cc2)cc1 ; c1ccc(-c2cc(-c3ccccc3)cc(N(c3ccccc3-c3cccc4sc5ccccc5c34)c3cccc4oc5c6ccccc6ccc5c34)c2)cc1 ; c1ccc(N(c2ccc(-c3ccccc3-n3c4ccccc4c4ccccc43)cc2)c2cccc3oc4c5ccccc5ccc4c23)c(-c2cccc3sc4ccccc4c23)c1]

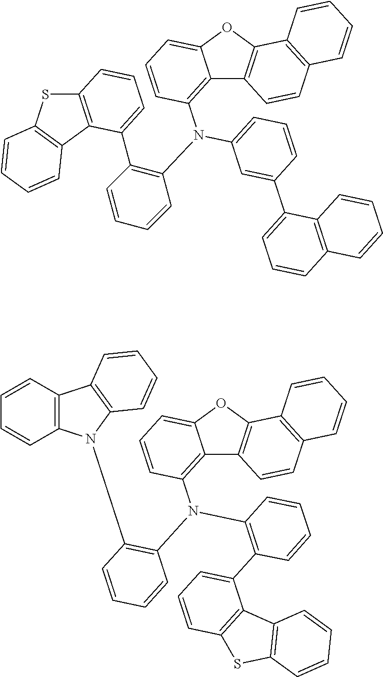 [SMILES: c1cc(-c2cccc3ccccc23)cc(N(c2ccccc2-c2cccc3sc4ccccc4c23)c2cccc3oc4c5ccccc5ccc4c23)c1 ; c1ccc(N(c2ccccc2-n2c3ccccc3c3ccccc32)c2cccc3oc4c5ccccc5ccc4c23)c(-c2cccc3sc4ccccc4c23)c1]

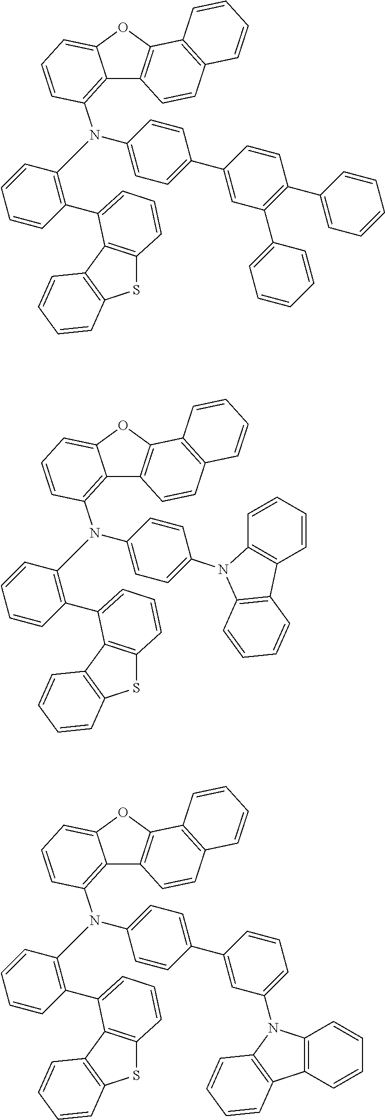 [SMILES: c1ccc(-c2ccc(-c3ccc(N(c4ccccc4-c4cccc5sc6ccccc6c45)c4cccc5oc6c7ccccc7ccc6c45)cc3)cc2-c2ccccc2)cc1 ; c1ccc(N(c2ccc(-n3c4ccccc4c4ccccc43)cc2)c2cccc3oc4c5ccccc5ccc4c23)c(-c2cccc3sc4ccccc4c23)c1 ; c1cc(-c2ccc(N(c3ccccc3-c3cccc4sc5ccccc5c34)c3cccc4oc5c6ccccc6ccc5c34)cc2)cc(-n2c3ccccc3c3ccccc32)c1]

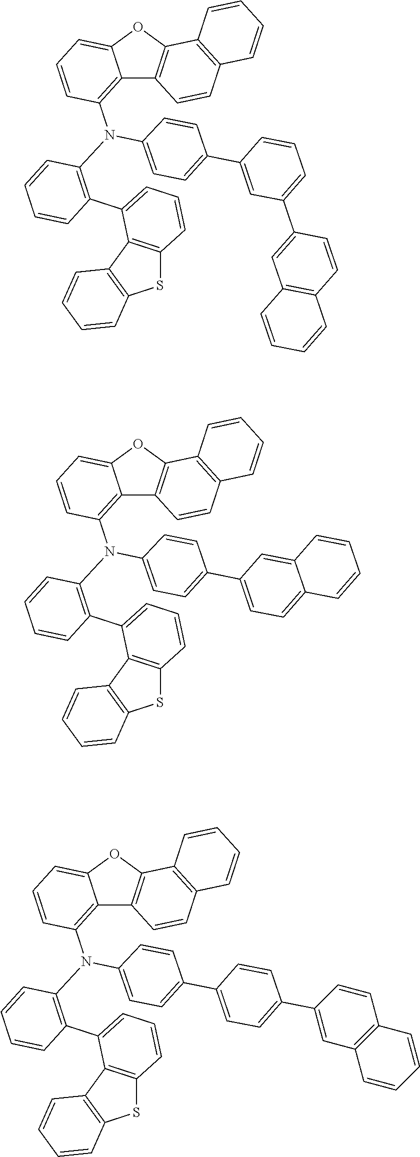 [SMILES: c1cc(-c2ccc(N(c3ccccc3-c3cccc4sc5ccccc5c34)c3cccc4oc5c6ccccc6ccc5c34)cc2)cc(-c2ccc3ccccc3c2)c1 ; c1ccc(N(c2ccc(-c3ccc4ccccc4c3)cc2)c2cccc3oc4c5ccccc5ccc4c23)c(-c2cccc3sc4ccccc4c23)c1 ; c1ccc(N(c2ccc(-c3ccc(-c4ccc5ccccc5c4)cc3)cc2)c2cccc3oc4c5ccccc5ccc4c23)c(-c2cccc3sc4ccccc4c23)c1]

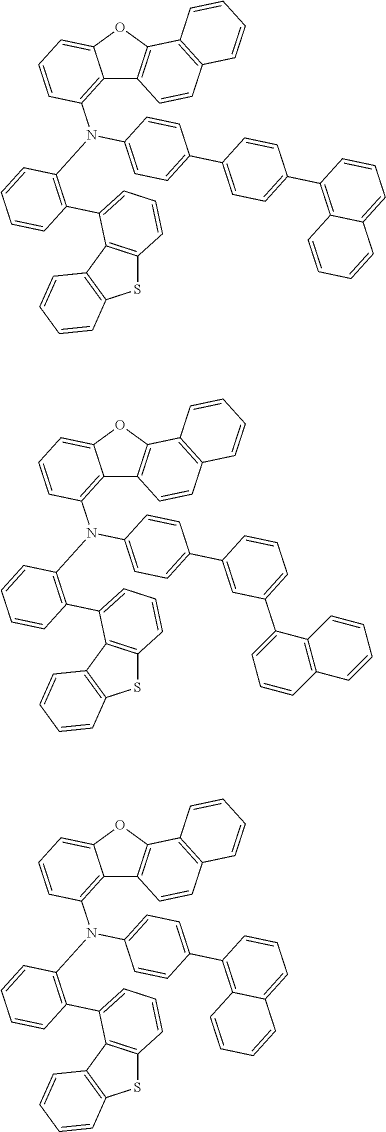 [SMILES: c1ccc(N(c2ccc(-c3ccc(-c4cccc5ccccc45)cc3)cc2)c2cccc3oc4c5ccccc5ccc4c23)c(-c2cccc3sc4ccccc4c23)c1 ; c1cc(-c2ccc(N(c3ccccc3-c3cccc4sc5ccccc5c34)c3cccc4oc5c6ccccc6ccc5c34)cc2)cc(-c2cccc3ccccc23)c1 ; c1ccc(N(c2ccc(-c3cccc4ccccc34)cc2)c2cccc3oc4c5ccccc5ccc4c23)c(-c2cccc3sc4ccccc4c23)c1]

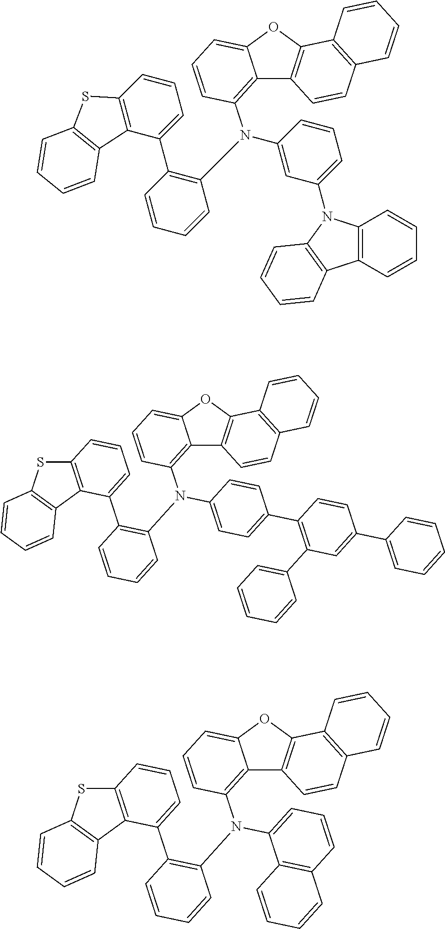 [SMILES: c1cc(N(c2ccccc2-c2cccc3sc4ccccc4c23)c2cccc3oc4c5ccccc5ccc4c23)cc(-n2c3ccccc3c3ccccc32)c1 ; c1ccc(-c2ccc(-c3ccc(N(c4ccccc4-c4cccc5sc6ccccc6c45)c4cccc5oc6c7ccccc7ccc6c45)cc3)c(-c3ccccc3)c2)cc1 ; c1ccc(N(c2cccc3ccccc23)c2cccc3oc4c5ccccc5ccc4c23)c(-c2cccc3sc4ccccc4c23)c1]

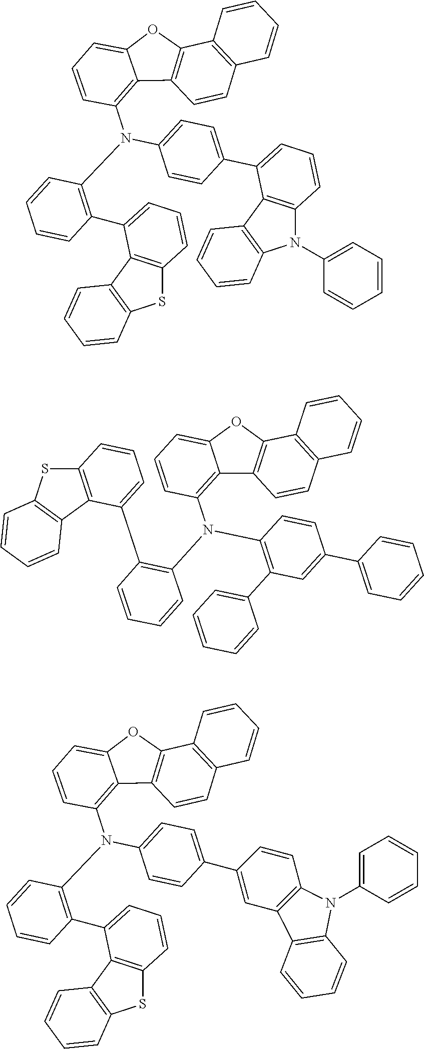 [SMILES: c1ccc(-n2c3ccccc3c3c(-c4ccc(N(c5ccccc5-c5cccc6sc7ccccc7c56)c5cccc6oc7c8ccccc8ccc7c56)cc4)cccc32)cc1 ; c1ccc(-c2ccc(N(c3ccccc3-c3cccc4sc5ccccc5c34)c3cccc4oc5c6ccccc6ccc5c34)c(-c3ccccc3)c2)cc1 ; c1ccc(-n2c3ccccc3c3cc(-c4ccc(N(c5ccccc5-c5cccc6sc7ccccc7c56)c5cccc6oc7c8ccccc8ccc7c56)cc4)ccc32)cc1]

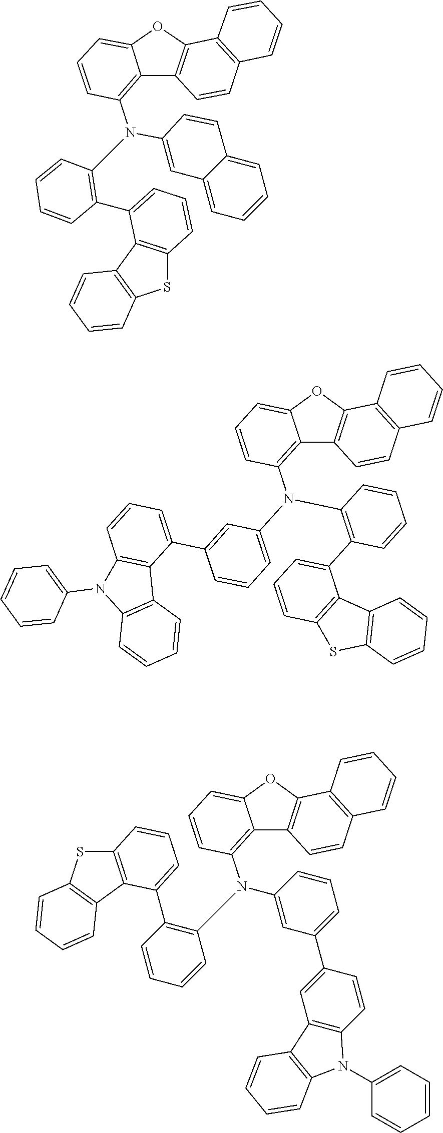 [SMILES: c1ccc(N(c2ccc3ccccc3c2)c2cccc3oc4c5ccccc5ccc4c23)c(-c2cccc3sc4ccccc4c23)c1 ; c1ccc(-n2c3ccccc3c3c(-c4cccc(N(c5ccccc5-c5cccc6sc7ccccc7c56)c5cccc6oc7c8ccccc8ccc7c56)c4)cccc32)cc1 ; c1ccc(-n2c3ccccc3c3cc(-c4cccc(N(c5ccccc5-c5cccc6sc7ccccc7c56)c5cccc6oc7c8ccccc8ccc7c56)c4)ccc32)cc1]

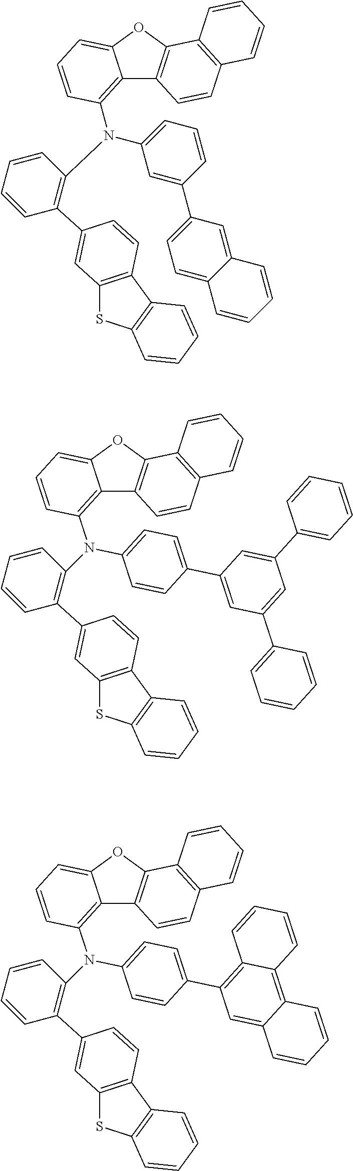

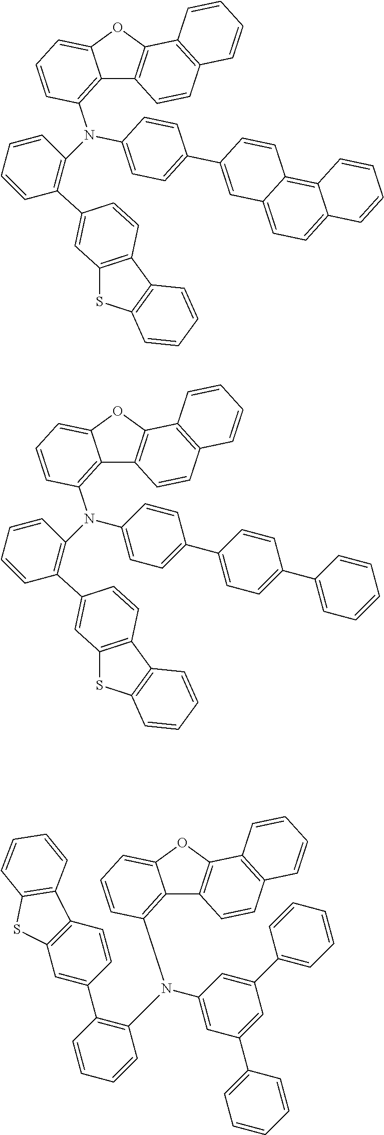 [SMILES: c1ccc(N(c2ccc(-c3ccc4c(ccc5ccccc54)c3)cc2)c2cccc3oc4c5ccccc5ccc4c23)c(-c2ccc3c(c2)sc2ccccc23)c1 ; c1ccc(-c2ccc(-c3ccc(N(c4ccccc4-c4ccc5c(c4)sc4ccccc45)c4cccc5oc6c7ccccc7ccc6c45)cc3)cc2)cc1 ; c1ccc(-c2cc(-c3ccccc3)cc(N(c3ccccc3-c3ccc4c(c3)sc3ccccc34)c3cccc4oc5c6ccccc6ccc5c34)c2)cc1]

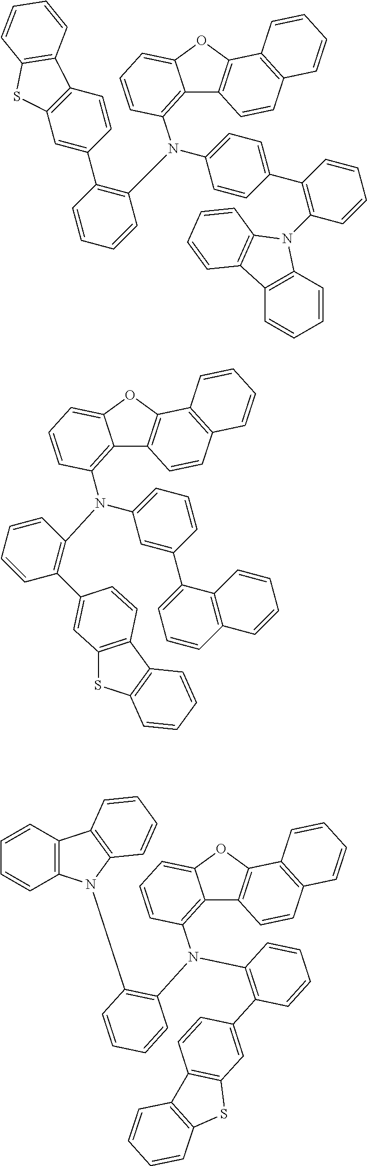 [SMILES: c1ccc(N(c2ccc(-c3ccccc3-n3c4ccccc4c4ccccc43)cc2)c2cccc3oc4c5ccccc5ccc4c23)c(-c2ccc3c(c2)sc2ccccc23)c1 ; c1cc(-c2cccc3ccccc23)cc(N(c2ccccc2-c2ccc3c(c2)sc2ccccc23)c2cccc3oc4c5ccccc5ccc4c23)c1 ; c1ccc(N(c2ccccc2-n2c3ccccc3c3ccccc32)c2cccc3oc4c5ccccc5ccc4c23)c(-c2ccc3c(c2)sc2ccccc23)c1]

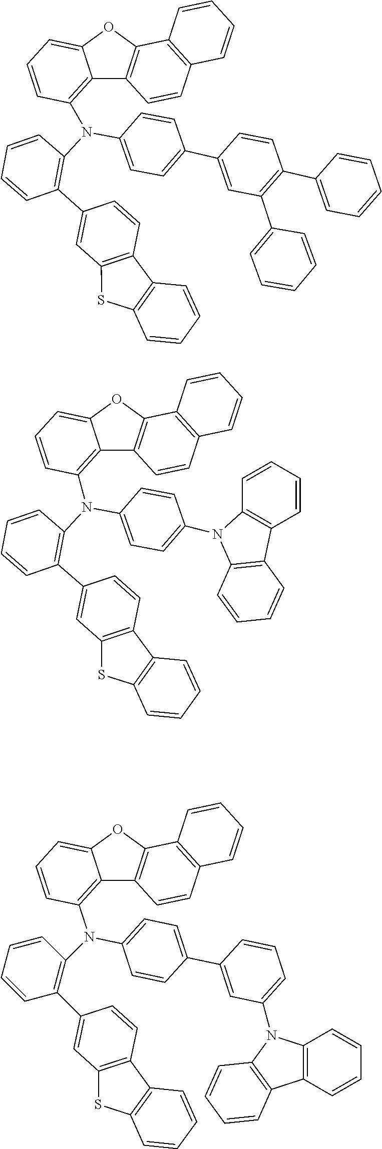

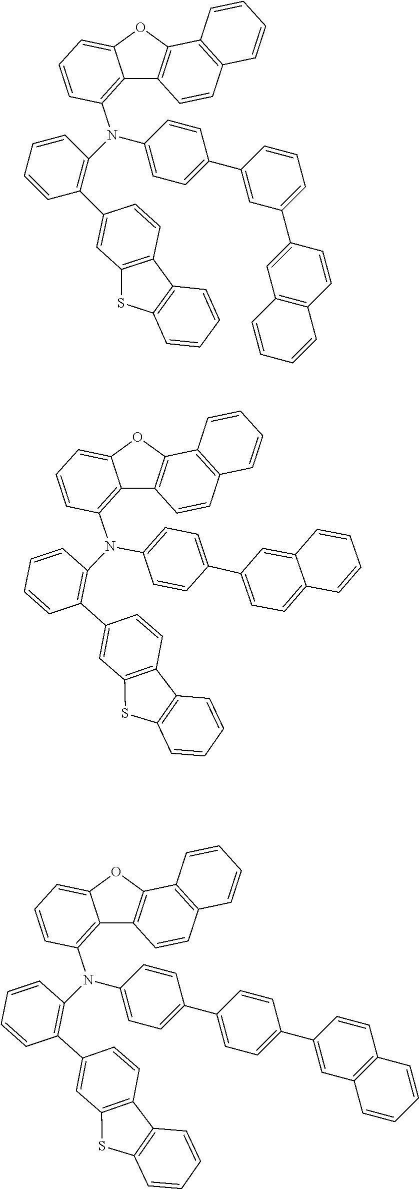 [SMILES: c1cc(-c2ccc(N(c3ccccc3-c3ccc4c(c3)sc3ccccc34)c3cccc4oc5c6ccccc6ccc5c34)cc2)cc(-c2ccc3ccccc3c2)c1 ; c1ccc(N(c2ccc(-c3ccc4ccccc4c3)cc2)c2cccc3oc4c5ccccc5ccc4c23)c(-c2ccc3c(c2)sc2ccccc23)c1 ; c1ccc(N(c2ccc(-c3ccc(-c4ccc5ccccc5c4)cc3)cc2)c2cccc3oc4c5ccccc5ccc4c23)c(-c2ccc3c(c2)sc2ccccc23)c1]

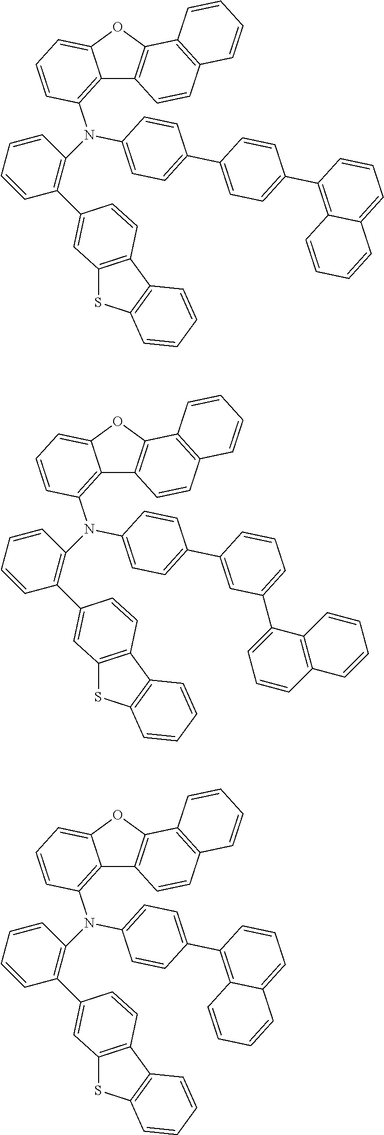 [SMILES: c1ccc(N(c2ccc(-c3ccc(-c4cccc5ccccc45)cc3)cc2)c2cccc3oc4c5ccccc5ccc4c23)c(-c2ccc3c(c2)sc2ccccc23)c1 ; c1cc(-c2ccc(N(c3ccccc3-c3ccc4c(c3)sc3ccccc34)c3cccc4oc5c6ccccc6ccc5c34)cc2)cc(-c2cccc3ccccc23)c1 ; c1ccc(N(c2ccc(-c3cccc4ccccc34)cc2)c2cccc3oc4c5ccccc5ccc4c23)c(-c2ccc3c(c2)sc2ccccc23)c1]

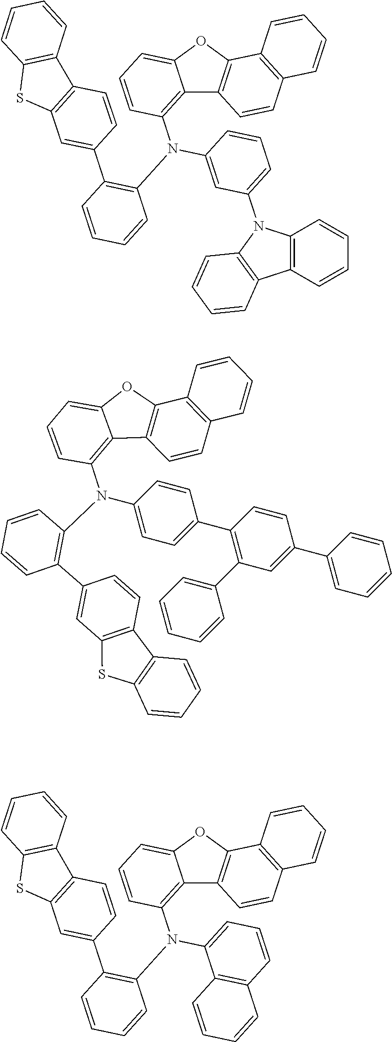

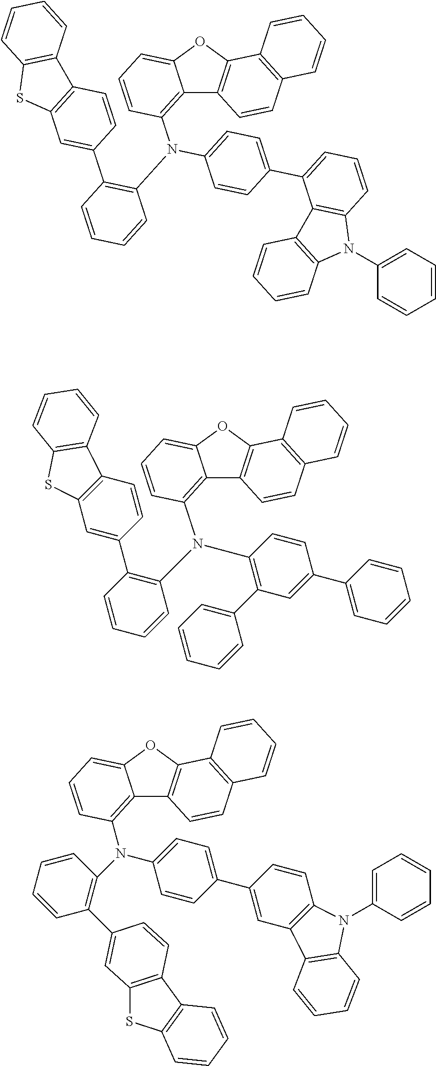 [SMILES: c1ccc(-n2c3ccccc3c3c(-c4ccc(N(c5ccccc5-c5ccc6c(c5)sc5ccccc56)c5cccc6oc7c8ccccc8ccc7c56)cc4)cccc32)cc1 ; c1ccc(-c2ccc(N(c3ccccc3-c3ccc4c(c3)sc3ccccc34)c3cccc4oc5c6ccccc6ccc5c34)c(-c3ccccc3)c2)cc1 ; c1ccc(-n2c3ccccc3c3cc(-c4ccc(N(c5ccccc5-c5ccc6c(c5)sc5ccccc56)c5cccc6oc7c8ccccc8ccc7c56)cc4)ccc32)cc1]

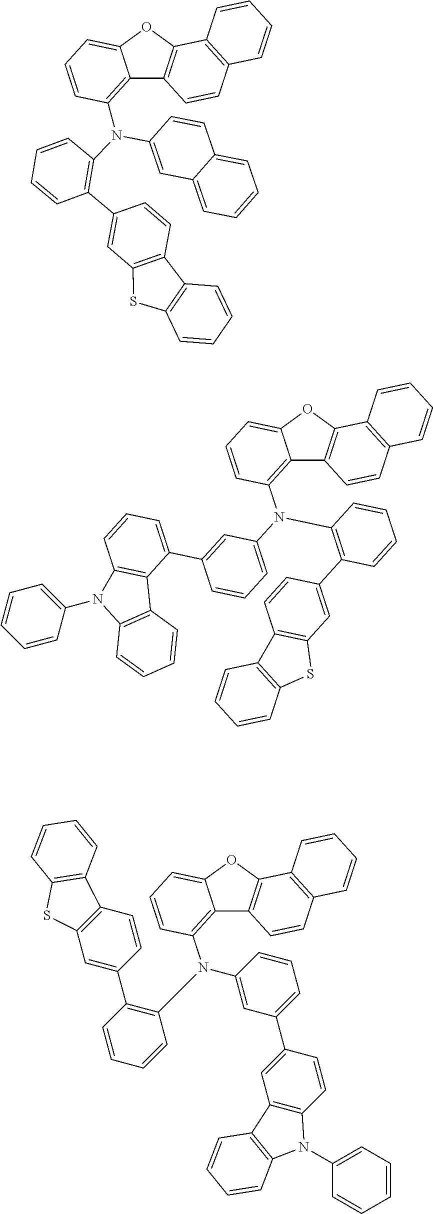 [SMILES: c1ccc(N(c2ccc3ccccc3c2)c2cccc3oc4c5ccccc5ccc4c23)c(-c2ccc3c(c2)sc2ccccc23)c1 ; c1ccc(-n2c3ccccc3c3c(-c4cccc(N(c5ccccc5-c5ccc6c(c5)sc5ccccc56)c5cccc6oc7c8ccccc8ccc7c56)c4)cccc32)cc1 ; c1ccc(-n2c3ccccc3c3cc(-c4cccc(N(c5ccccc5-c5ccc6c(c5)sc5ccccc56)c5cccc6oc7c8ccccc8ccc7c56)c4)ccc32)cc1]

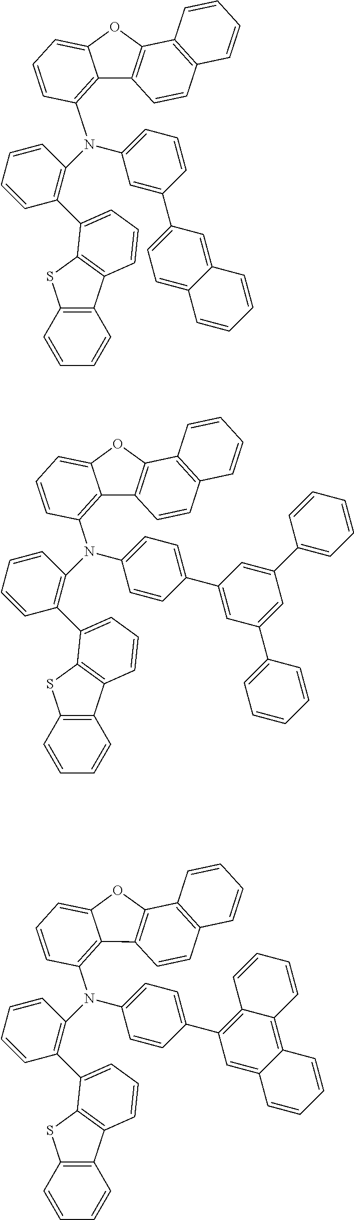 [SMILES: c1cc(-c2ccc3ccccc3c2)cc(N(c2ccccc2-c2cccc3c2sc2ccccc23)c2cccc3oc4c5ccccc5ccc4c23)c1 ; c1ccc(-c2cc(-c3ccccc3)cc(-c3ccc(N(c4ccccc4-c4cccc5c4sc4ccccc45)c4cccc5oc6c7ccccc7ccc6c45)cc3)c2)cc1 ; c1ccc(N(c2ccc(-c3cc4ccccc4c4ccccc34)cc2)c2cccc3oc4c5ccccc5ccc4c23)c(-c2cccc3c2sc2ccccc23)c1]

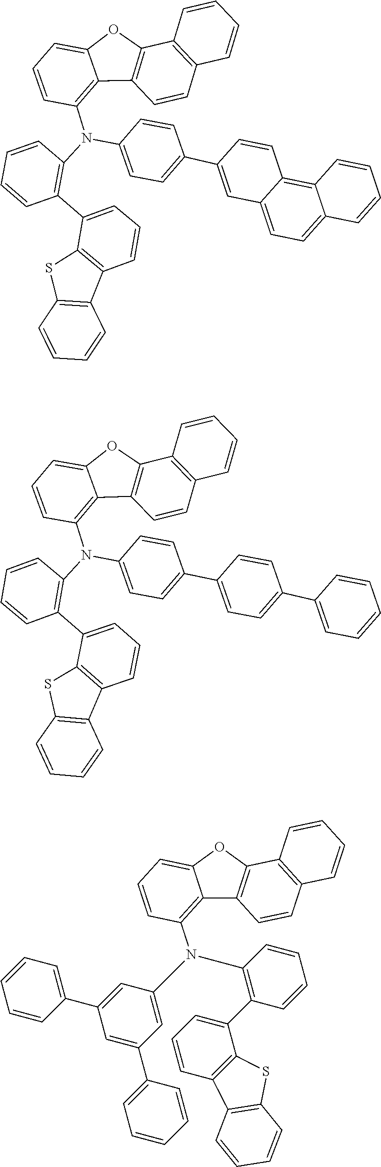 [SMILES: c1ccc(N(c2ccc(-c3ccc4c(ccc5ccccc54)c3)cc2)c2cccc3oc4c5ccccc5ccc4c23)c(-c2cccc3c2sc2ccccc23)c1 ; c1ccc(-c2ccc(-c3ccc(N(c4ccccc4-c4cccc5c4sc4ccccc45)c4cccc5oc6c7ccccc7ccc6c45)cc3)cc2)cc1 ; c1ccc(-c2cc(-c3ccccc3)cc(N(c3ccccc3-c3cccc4c3sc3ccccc34)c3cccc4oc5c6ccccc6ccc5c34)c2)cc1]

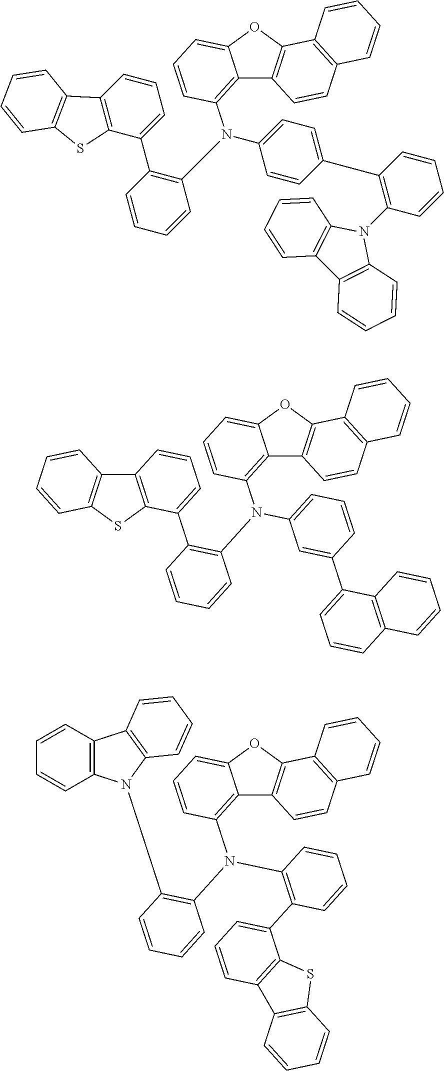 [SMILES: c1ccc(N(c2ccc(-c3ccccc3-n3c4ccccc4c4ccccc43)cc2)c2cccc3oc4c5ccccc5ccc4c23)c(-c2cccc3c2sc2ccccc23)c1 ; c1cc(-c2cccc3ccccc23)cc(N(c2ccccc2-c2cccc3c2sc2ccccc23)c2cccc3oc4c5ccccc5ccc4c23)c1 ; c1ccc(N(c2ccccc2-n2c3ccccc3c3ccccc32)c2cccc3oc4c5ccccc5ccc4c23)c(-c2cccc3c2sc2ccccc23)c1]

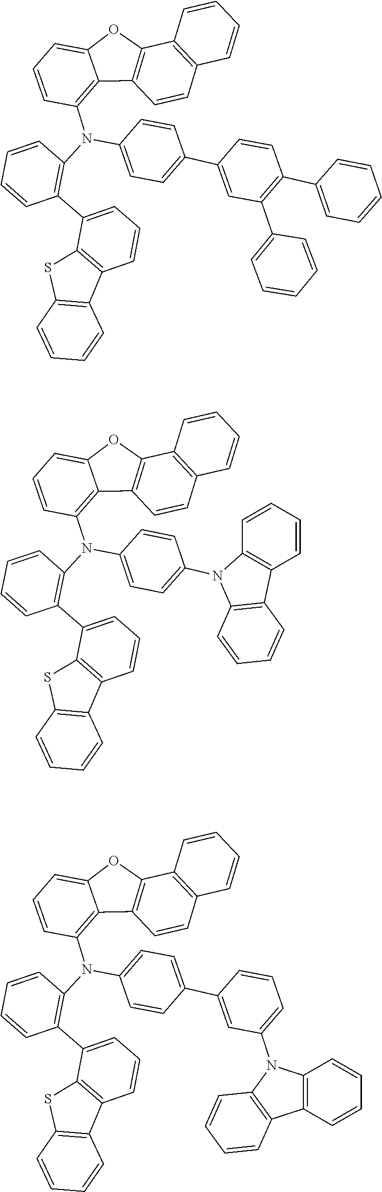 [SMILES: c1ccc(-c2ccc(-c3ccc(N(c4ccccc4-c4cccc5c4sc4ccccc45)c4cccc5oc6c7ccccc7ccc6c45)cc3)cc2-c2ccccc2)cc1 ; c1ccc(N(c2ccc(-n3c4ccccc4c4ccccc43)cc2)c2cccc3oc4c5ccccc5ccc4c23)c(-c2cccc3c2sc2ccccc23)c1 ; c1cc(-c2ccc(N(c3ccccc3-c3cccc4c3sc3ccccc34)c3cccc4oc5c6ccccc6ccc5c34)cc2)cc(-n2c3ccccc3c3ccccc32)c1]

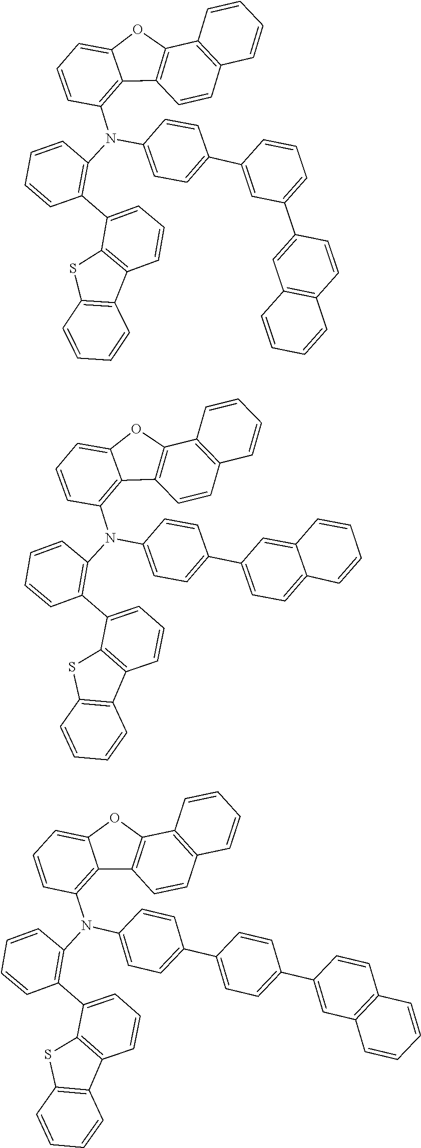 [SMILES: c1cc(-c2ccc(N(c3ccccc3-c3cccc4c3sc3ccccc34)c3cccc4oc5c6ccccc6ccc5c34)cc2)cc(-c2ccc3ccccc3c2)c1 ; c1ccc(N(c2ccc(-c3ccc4ccccc4c3)cc2)c2cccc3oc4c5ccccc5ccc4c23)c(-c2cccc3c2sc2ccccc23)c1 ; c1ccc(N(c2ccc(-c3ccc(-c4ccc5ccccc5c4)cc3)cc2)c2cccc3oc4c5ccccc5ccc4c23)c(-c2cccc3c2sc2ccccc23)c1]

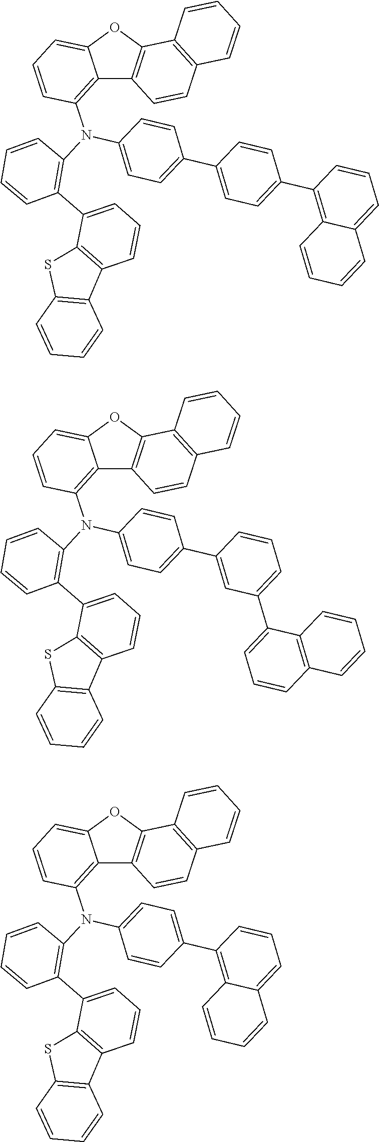 [SMILES: c1ccc(N(c2ccc(-c3ccc(-c4cccc5ccccc45)cc3)cc2)c2cccc3oc4c5ccccc5ccc4c23)c(-c2cccc3c2sc2ccccc23)c1 ; c1cc(-c2ccc(N(c3ccccc3-c3cccc4c3sc3ccccc34)c3cccc4oc5c6ccccc6ccc5c34)cc2)cc(-c2cccc3ccccc23)c1 ; c1ccc(N(c2ccc(-c3cccc4ccccc34)cc2)c2cccc3oc4c5ccccc5ccc4c23)c(-c2cccc3c2sc2ccccc23)c1]

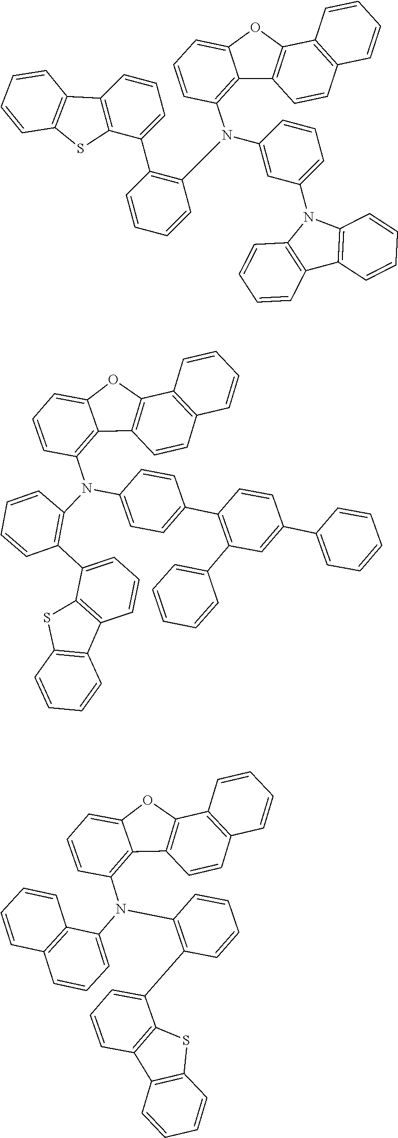 [SMILES: c1cc(N(c2ccccc2-c2cccc3c2sc2ccccc23)c2cccc3oc4c5ccccc5ccc4c23)cc(-n2c3ccccc3c3ccccc32)c1 ; c1ccc(-c2ccc(-c3ccc(N(c4ccccc4-c4cccc5c4sc4ccccc45)c4cccc5oc6c7ccccc7ccc6c45)cc3)c(-c3ccccc3)c2)cc1 ; c1ccc(N(c2cccc3ccccc23)c2cccc3oc4c5ccccc5ccc4c23)c(-c2cccc3c2sc2ccccc23)c1]

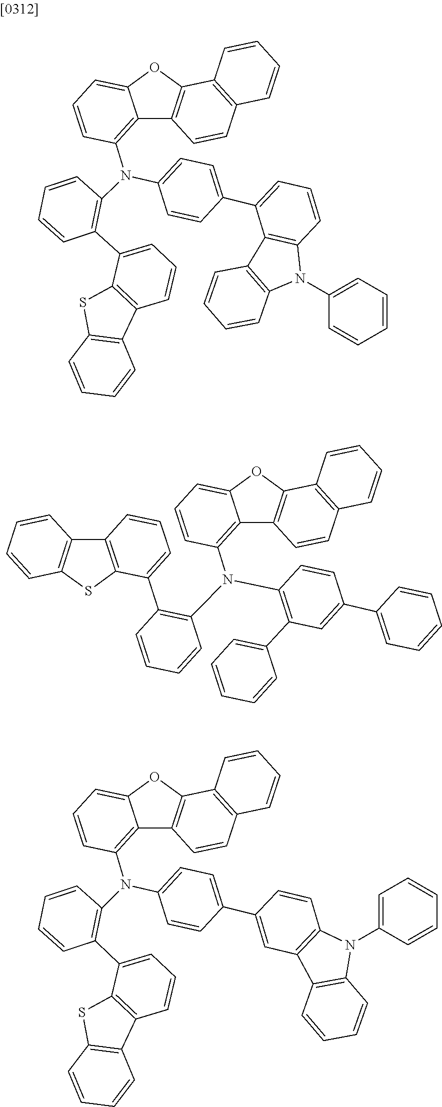 [SMILES: c1ccc(-n2c3ccccc3c3c(-c4ccc(N(c5ccccc5-c5cccc6c5sc5ccccc56)c5cccc6oc7c8ccccc8ccc7c56)cc4)cccc32)cc1 ; c1ccc(-c2ccc(N(c3ccccc3-c3cccc4c3sc3ccccc34)c3cccc4oc5c6ccccc6ccc5c34)c(-c3ccccc3)c2)cc1 ; c1ccc(-n2c3ccccc3c3cc(-c4ccc(N(c5ccccc5-c5cccc6c5sc5ccccc56)c5cccc6oc7c8ccccc8ccc7c56)cc4)ccc32)cc1]

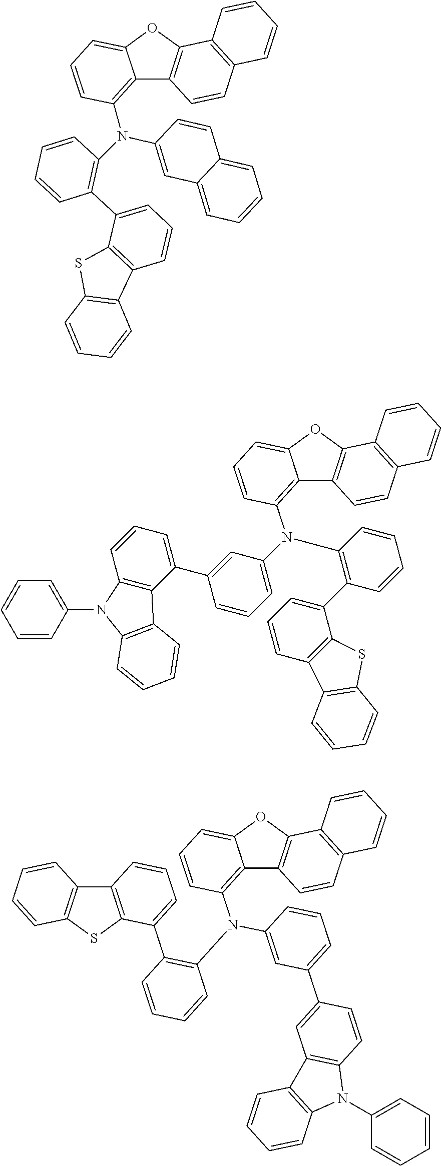 [SMILES: c1ccc(N(c2ccc3ccccc3c2)c2cccc3oc4c5ccccc5ccc4c23)c(-c2cccc3c2sc2ccccc23)c1 ; c1ccc(-n2c3ccccc3c3c(-c4cccc(N(c5ccccc5-c5cccc6c5sc5ccccc56)c5cccc6oc7c8ccccc8ccc7c56)c4)cccc32)cc1 ; c1ccc(-n2c3ccccc3c3cc(-c4cccc(N(c5ccccc5-c5cccc6c5sc5ccccc56)c5cccc6oc7c8ccccc8ccc7c56)c4)ccc32)cc1]

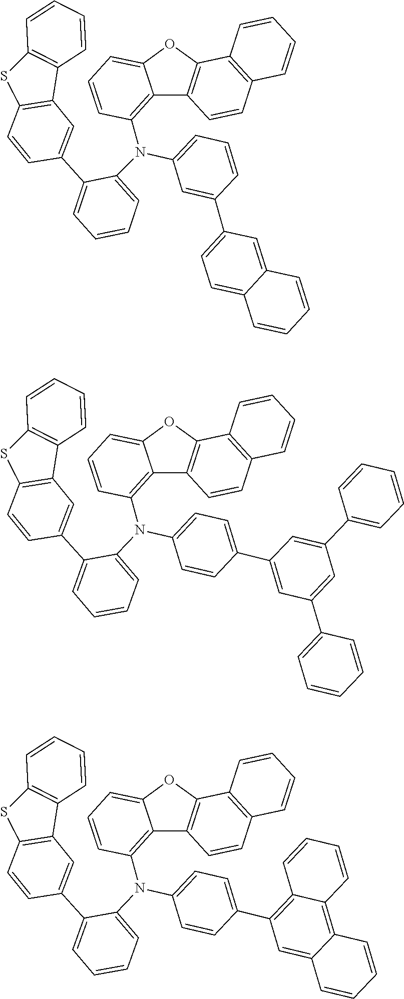 [SMILES: c1cc(-c2ccc3ccccc3c2)cc(N(c2ccccc2-c2ccc3sc4ccccc4c3c2)c2cccc3oc4c5ccccc5ccc4c23)c1 ; c1ccc(-c2cc(-c3ccccc3)cc(-c3ccc(N(c4ccccc4-c4ccc5sc6ccccc6c5c4)c4cccc5oc6c7ccccc7ccc6c45)cc3)c2)cc1 ; c1ccc(N(c2ccc(-c3cc4ccccc4c4ccccc34)cc2)c2cccc3oc4c5ccccc5ccc4c23)c(-c2ccc3sc4ccccc4c3c2)c1]

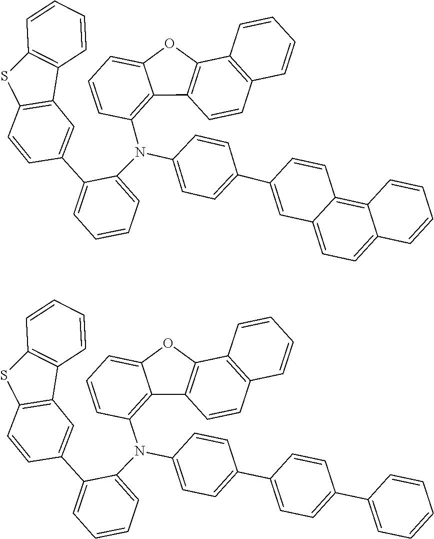 [SMILES: c1ccc(N(c2ccc(-c3ccc4c(ccc5ccccc54)c3)cc2)c2cccc3oc4c5ccccc5ccc4c23)c(-c2ccc3sc4ccccc4c3c2)c1 ; c1ccc(-c2ccc(-c3ccc(N(c4ccccc4-c4ccc5sc6ccccc6c5c4)c4cccc5oc6c7ccccc7ccc6c45)cc3)cc2)cc1]

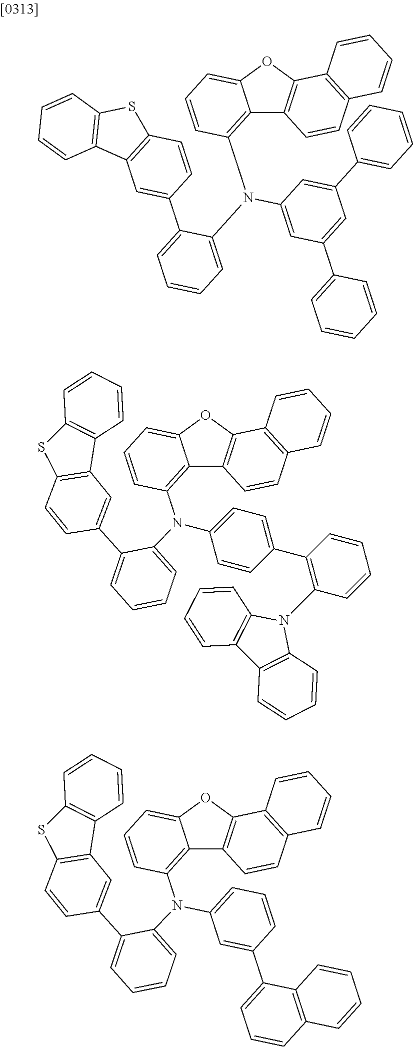 [SMILES: c1ccc(-c2cc(-c3ccccc3)cc(N(c3ccccc3-c3ccc4sc5ccccc5c4c3)c3cccc4oc5c6ccccc6ccc5c34)c2)cc1 ; c1ccc(N(c2ccc(-c3ccccc3-n3c4ccccc4c4ccccc43)cc2)c2cccc3oc4c5ccccc5ccc4c23)c(-c2ccc3sc4ccccc4c3c2)c1 ; c1cc(-c2cccc3ccccc23)cc(N(c2ccccc2-c2ccc3sc4ccccc4c3c2)c2cccc3oc4c5ccccc5ccc4c23)c1]

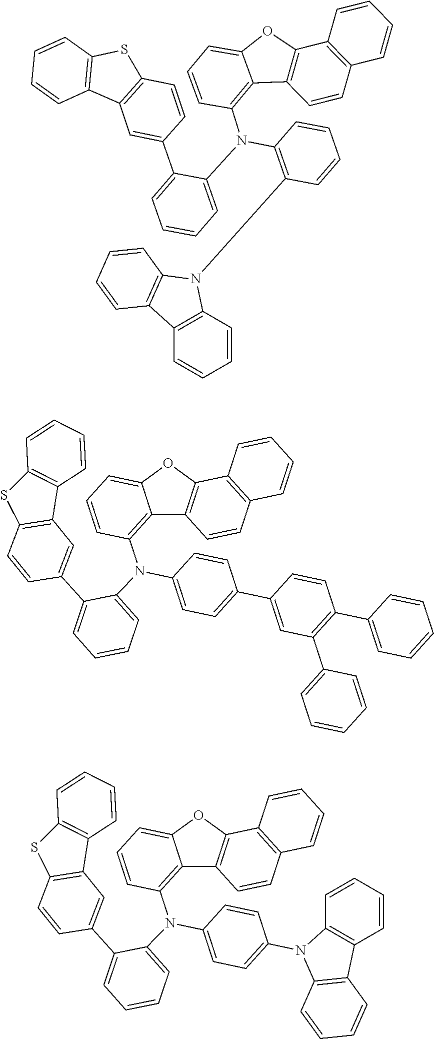 [SMILES: c1ccc(N(c2ccccc2-n2c3ccccc3c3ccccc32)c2cccc3oc4c5ccccc5ccc4c23)c(-c2ccc3sc4ccccc4c3c2)c1 ; c1ccc(-c2ccc(-c3ccc(N(c4ccccc4-c4ccc5sc6ccccc6c5c4)c4cccc5oc6c7ccccc7ccc6c45)cc3)cc2-c2ccccc2)cc1 ; c1ccc(N(c2ccc(-n3c4ccccc4c4ccccc43)cc2)c2cccc3oc4c5ccccc5ccc4c23)c(-c2ccc3sc4ccccc4c3c2)c1]

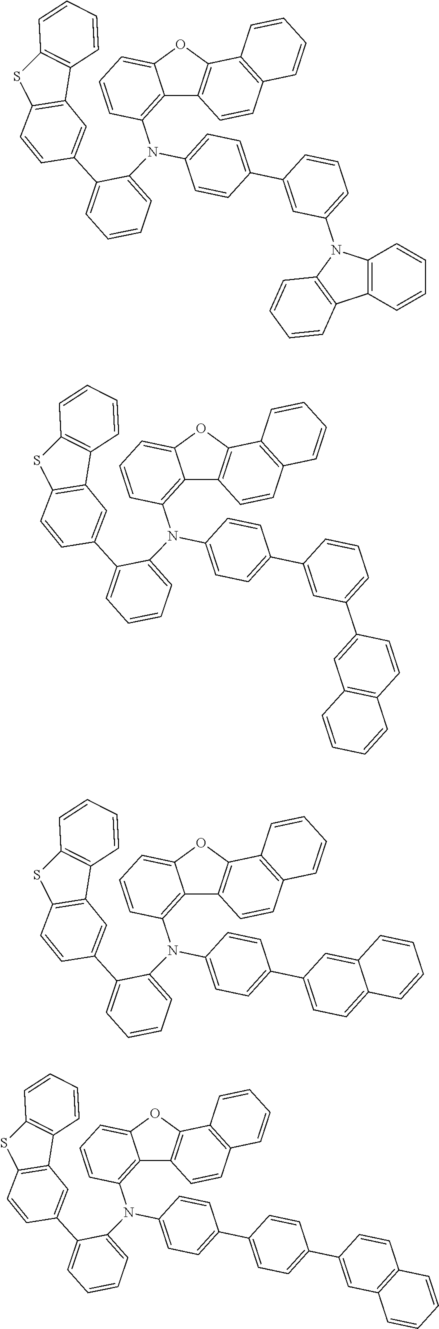 [SMILES: c1cc(-c2ccc(N(c3ccccc3-c3ccc4sc5ccccc5c4c3)c3cccc4oc5c6ccccc6ccc5c34)cc2)cc(-n2c3ccccc3c3ccccc32)c1 ; c1cc(-c2ccc(N(c3ccccc3-c3ccc4sc5ccccc5c4c3)c3cccc4oc5c6ccccc6ccc5c34)cc2)cc(-c2ccc3ccccc3c2)c1 ; c1ccc(N(c2ccc(-c3ccc4ccccc4c3)cc2)c2cccc3oc4c5ccccc5ccc4c23)c(-c2ccc3sc4ccccc4c3c2)c1 ; c1ccc(N(c2ccc(-c3ccc(-c4ccc5ccccc5c4)cc3)cc2)c2cccc3oc4c5ccccc5ccc4c23)c(-c2ccc3sc4ccccc4c3c2)c1]

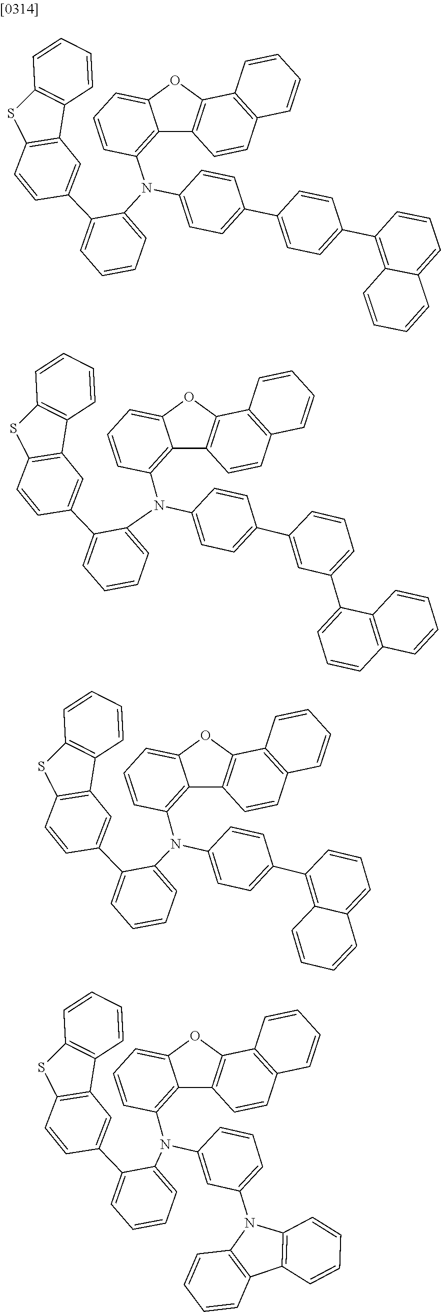 [SMILES: c1ccc(N(c2ccc(-c3ccc(-c4cccc5ccccc45)cc3)cc2)c2cccc3oc4c5ccccc5ccc4c23)c(-c2ccc3sc4ccccc4c3c2)c1 ; c1cc(-c2ccc(N(c3ccccc3-c3ccc4sc5ccccc5c4c3)c3cccc4oc5c6ccccc6ccc5c34)cc2)cc(-c2cccc3ccccc23)c1 ; c1ccc(N(c2ccc(-c3cccc4ccccc34)cc2)c2cccc3oc4c5ccccc5ccc4c23)c(-c2ccc3sc4ccccc4c3c2)c1 ; c1cc(N(c2ccccc2-c2ccc3sc4ccccc4c3c2)c2cccc3oc4c5ccccc5ccc4c23)cc(-n2c3ccccc3c3ccccc32)c1]

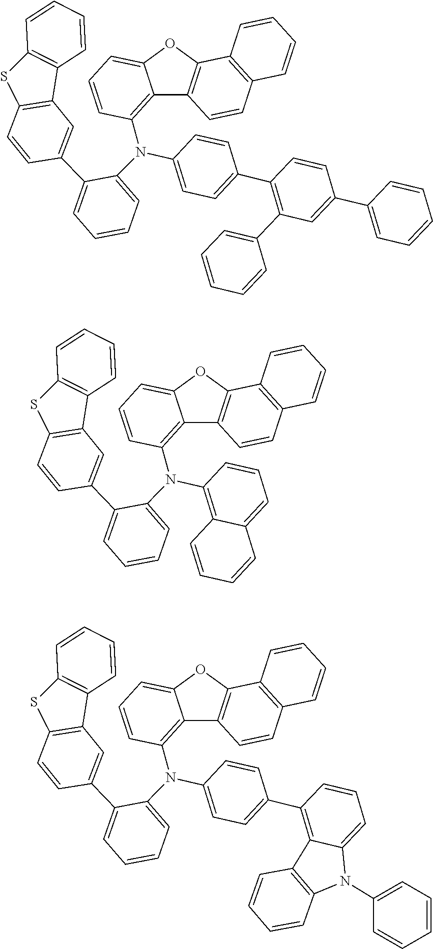 [SMILES: c1ccc(-c2ccc(-c3ccc(N(c4ccccc4-c4ccc5sc6ccccc6c5c4)c4cccc5oc6c7ccccc7ccc6c45)cc3)c(-c3ccccc3)c2)cc1 ; c1ccc(N(c2cccc3ccccc23)c2cccc3oc4c5ccccc5ccc4c23)c(-c2ccc3sc4ccccc4c3c2)c1 ; c1ccc(-n2c3ccccc3c3c(-c4ccc(N(c5ccccc5-c5ccc6sc7ccccc7c6c5)c5cccc6oc7c8ccccc8ccc7c56)cc4)cccc32)cc1]

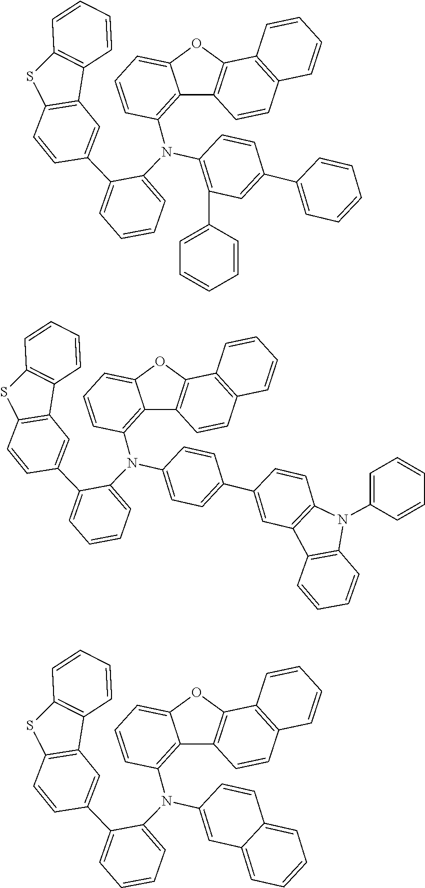 [SMILES: c1ccc(-c2ccc(N(c3ccccc3-c3ccc4sc5ccccc5c4c3)c3cccc4oc5c6ccccc6ccc5c34)c(-c3ccccc3)c2)cc1 ; c1ccc(-n2c3ccccc3c3cc(-c4ccc(N(c5ccccc5-c5ccc6sc7ccccc7c6c5)c5cccc6oc7c8ccccc8ccc7c56)cc4)ccc32)cc1 ; c1ccc(N(c2ccc3ccccc3c2)c2cccc3oc4c5ccccc5ccc4c23)c(-c2ccc3sc4ccccc4c3c2)c1]

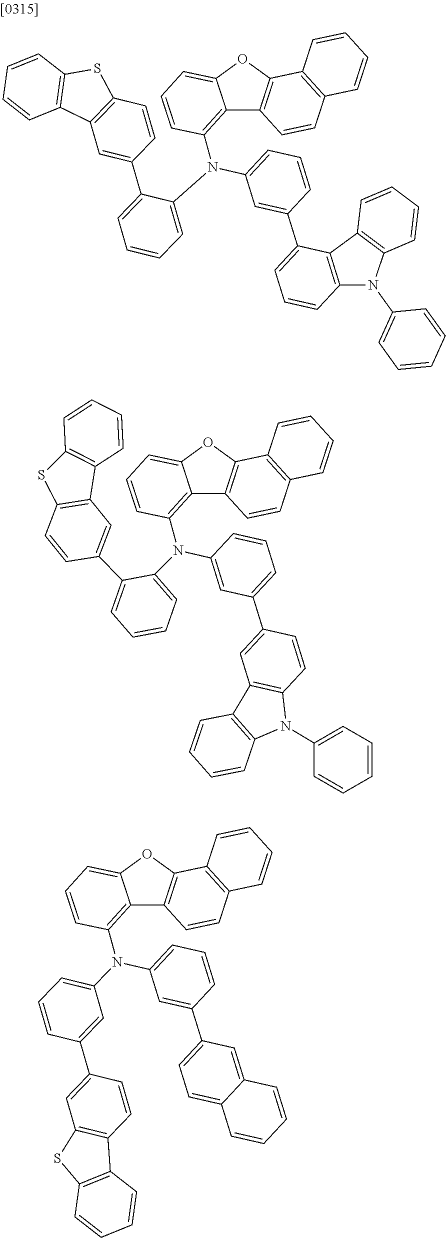

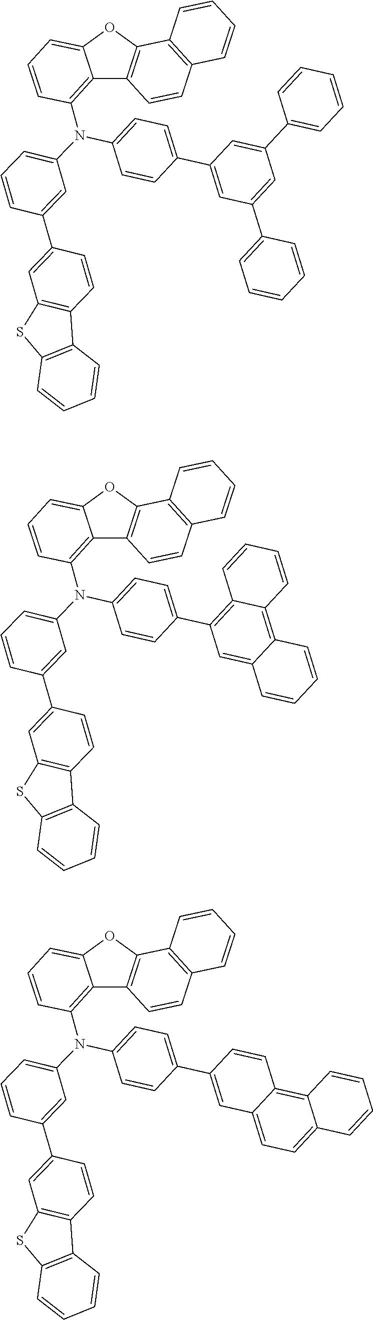

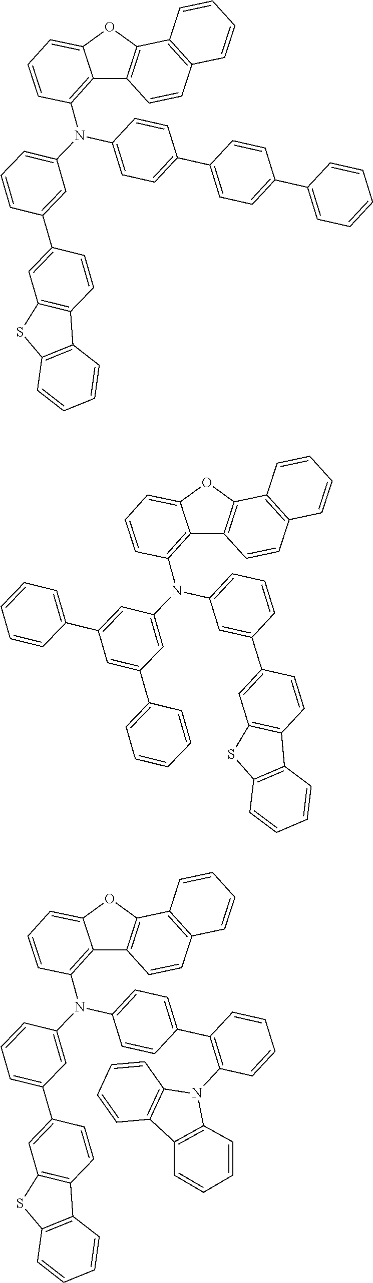 [SMILES: c1ccc(-c2ccc(-c3ccc(N(c4cccc(-c5ccc6c(c5)sc5ccccc56)c4)c4cccc5oc6c7ccccc7ccc6c45)cc3)cc2)cc1 ; c1ccc(-c2cc(-c3ccccc3)cc(N(c3cccc(-c4ccc5c(c4)sc4ccccc45)c3)c3cccc4oc5c6ccccc6ccc5c34)c2)cc1 ; c1cc(-c2ccc3c(c2)sc2ccccc23)cc(N(c2ccc(-c3ccccc3-n3c4ccccc4c4ccccc43)cc2)c2cccc3oc4c5ccccc5ccc4c23)c1]

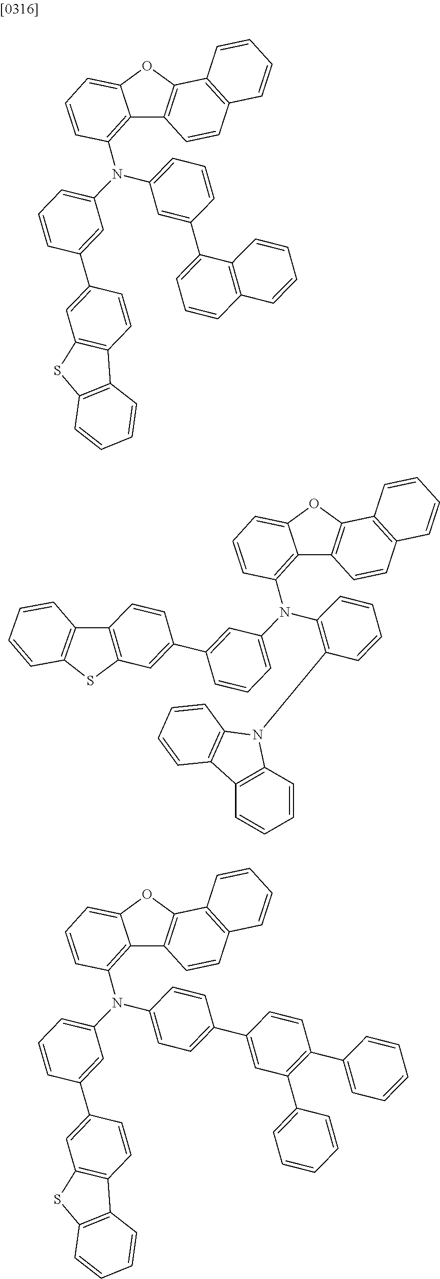

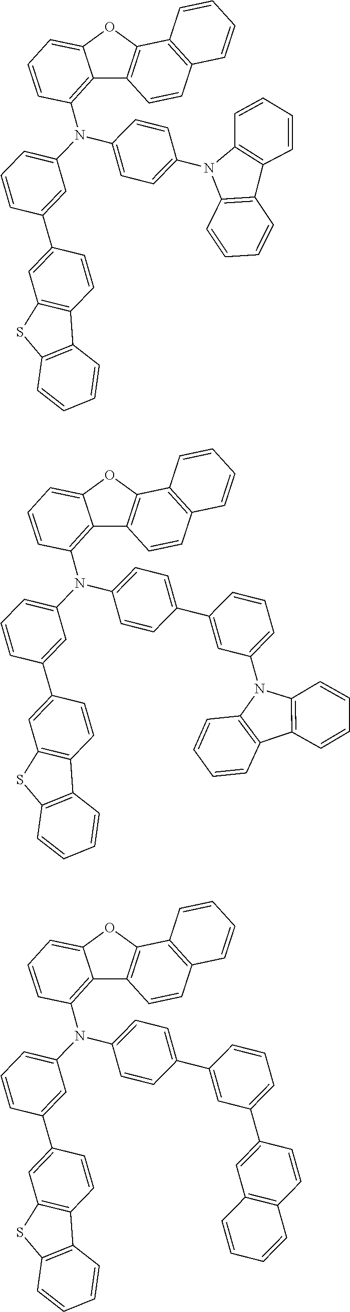 [SMILES: c1cc(-c2ccc3c(c2)sc2ccccc23)cc(N(c2ccc(-n3c4ccccc4c4ccccc43)cc2)c2cccc3oc4c5ccccc5ccc4c23)c1 ; c1cc(-c2ccc3c(c2)sc2ccccc23)cc(N(c2ccc(-c3cccc(-n4c5ccccc5c5ccccc54)c3)cc2)c2cccc3oc4c5ccccc5ccc4c23)c1 ; c1cc(-c2ccc(N(c3cccc(-c4ccc5c(c4)sc4ccccc45)c3)c3cccc4oc5c6ccccc6ccc5c34)cc2)cc(-c2ccc3ccccc3c2)c1]

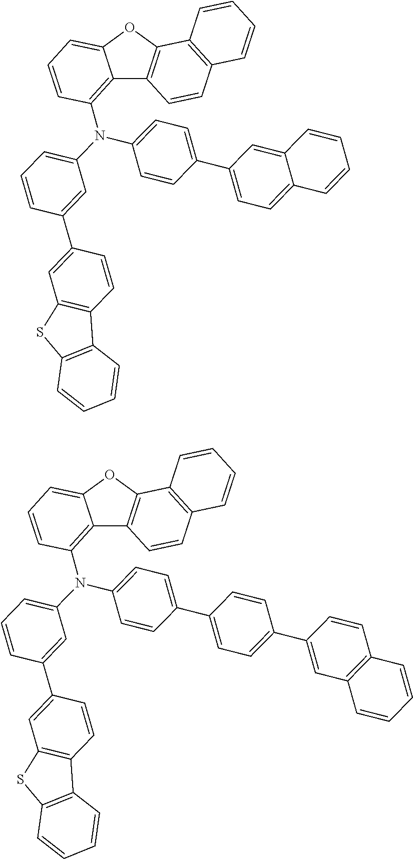 [SMILES: c1cc(-c2ccc3c(c2)sc2ccccc23)cc(N(c2ccc(-c3ccc4ccccc4c3)cc2)c2cccc3oc4c5ccccc5ccc4c23)c1 ; c1cc(-c2ccc3c(c2)sc2ccccc23)cc(N(c2ccc(-c3ccc(-c4ccc5ccccc5c4)cc3)cc2)c2cccc3oc4c5ccccc5ccc4c23)c1]

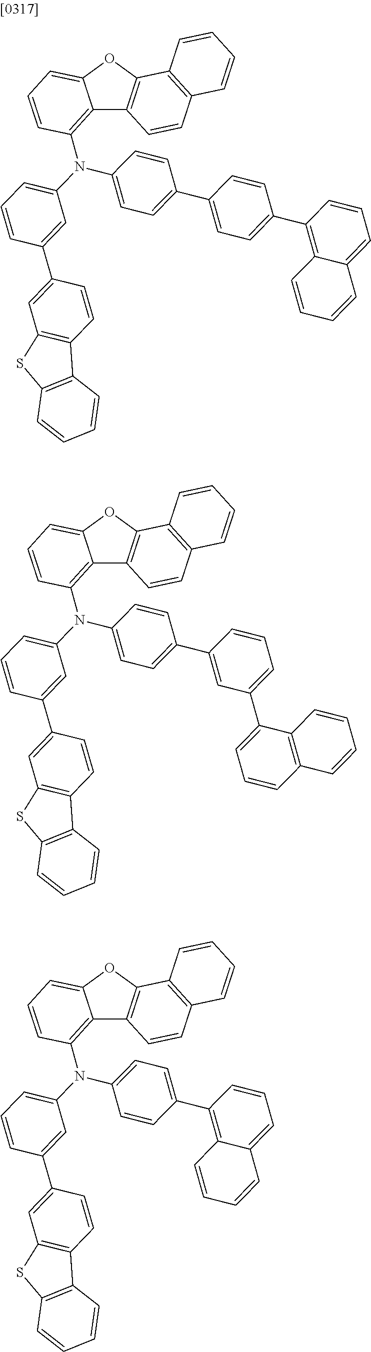 [SMILES: c1cc(-c2ccc3c(c2)sc2ccccc23)cc(N(c2ccc(-c3ccc(-c4cccc5ccccc45)cc3)cc2)c2cccc3oc4c5ccccc5ccc4c23)c1 ; c1cc(-c2ccc(N(c3cccc(-c4ccc5c(c4)sc4ccccc45)c3)c3cccc4oc5c6ccccc6ccc5c34)cc2)cc(-c2cccc3ccccc23)c1 ; c1cc(-c2ccc3c(c2)sc2ccccc23)cc(N(c2ccc(-c3cccc4ccccc34)cc2)c2cccc3oc4c5ccccc5ccc4c23)c1]

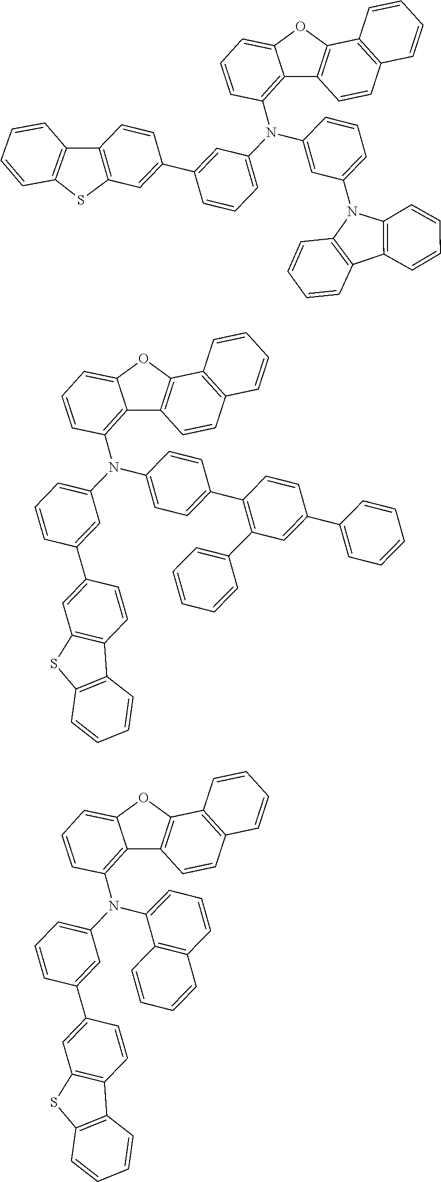 [SMILES: c1cc(-c2ccc3c(c2)sc2ccccc23)cc(N(c2cccc(-n3c4ccccc4c4ccccc43)c2)c2cccc3oc4c5ccccc5ccc4c23)c1 ; c1ccc(-c2ccc(-c3ccc(N(c4cccc(-c5ccc6c(c5)sc5ccccc56)c4)c4cccc5oc6c7ccccc7ccc6c45)cc3)c(-c3ccccc3)c2)cc1 ; c1cc(-c2ccc3c(c2)sc2ccccc23)cc(N(c2cccc3ccccc23)c2cccc3oc4c5ccccc5ccc4c23)c1]

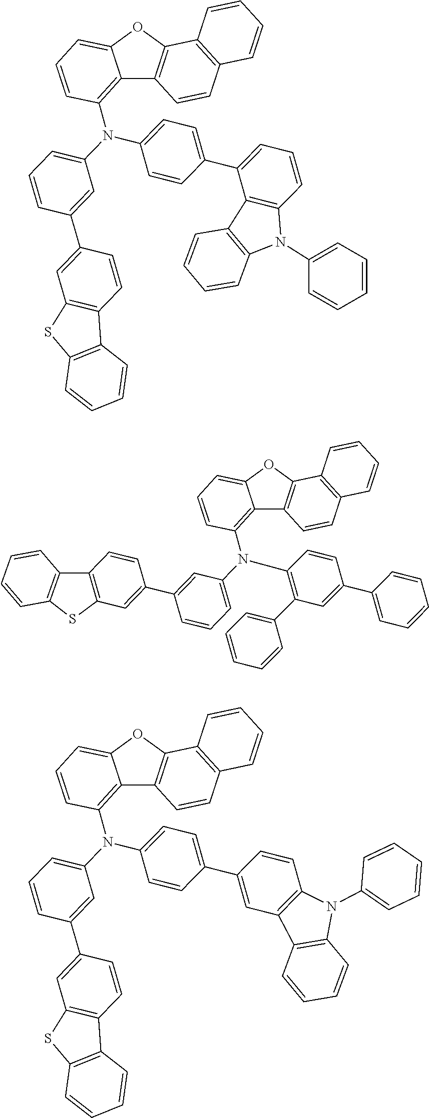

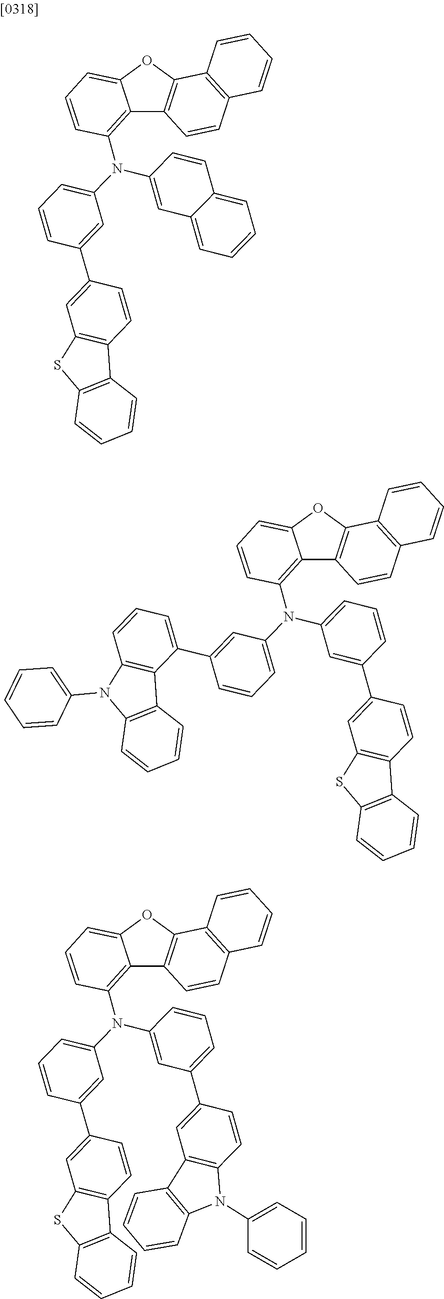 [SMILES: c1cc(-c2ccc3c(c2)sc2ccccc23)cc(N(c2ccc3ccccc3c2)c2cccc3oc4c5ccccc5ccc4c23)c1 ; c1ccc(-n2c3ccccc3c3c(-c4cccc(N(c5cccc(-c6ccc7c(c6)sc6ccccc67)c5)c5cccc6oc7c8ccccc8ccc7c56)c4)cccc32)cc1 ; c1ccc(-n2c3ccccc3c3cc(-c4cccc(N(c5cccc(-c6ccc7c(c6)sc6ccccc67)c5)c5cccc6oc7c8ccccc8ccc7c56)c4)ccc32)cc1]

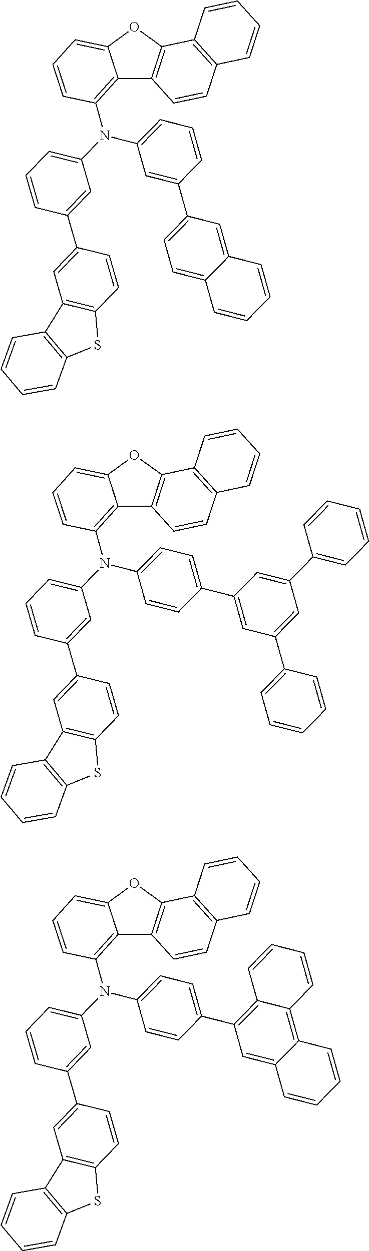 [SMILES: c1cc(-c2ccc3ccccc3c2)cc(N(c2cccc(-c3ccc4sc5ccccc5c4c3)c2)c2cccc3oc4c5ccccc5ccc4c23)c1 ; c1ccc(-c2cc(-c3ccccc3)cc(-c3ccc(N(c4cccc(-c5ccc6sc7ccccc7c6c5)c4)c4cccc5oc6c7ccccc7ccc6c45)cc3)c2)cc1 ; c1cc(-c2ccc3sc4ccccc4c3c2)cc(N(c2ccc(-c3cc4ccccc4c4ccccc34)cc2)c2cccc3oc4c5ccccc5ccc4c23)c1]

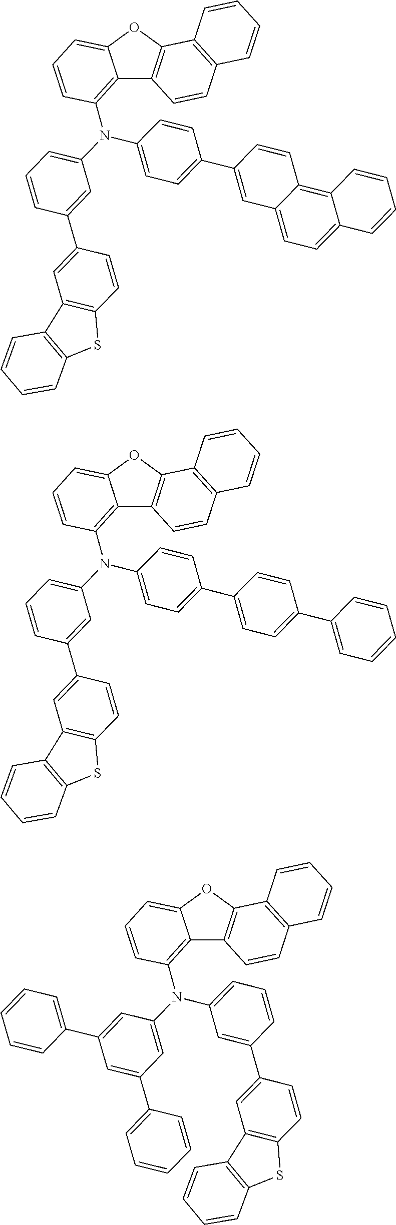 [SMILES: c1cc(-c2ccc3sc4ccccc4c3c2)cc(N(c2ccc(-c3ccc4c(ccc5ccccc54)c3)cc2)c2cccc3oc4c5ccccc5ccc4c23)c1 ; c1ccc(-c2ccc(-c3ccc(N(c4cccc(-c5ccc6sc7ccccc7c6c5)c4)c4cccc5oc6c7ccccc7ccc6c45)cc3)cc2)cc1 ; c1ccc(-c2cc(-c3ccccc3)cc(N(c3cccc(-c4ccc5sc6ccccc6c5c4)c3)c3cccc4oc5c6ccccc6ccc5c34)c2)cc1]

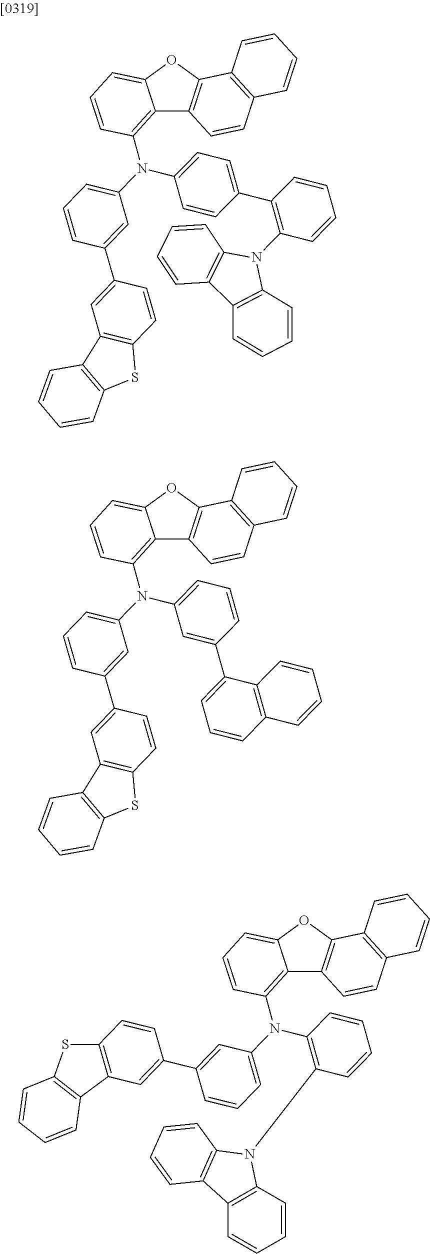 [SMILES: c1cc(-c2ccc3sc4ccccc4c3c2)cc(N(c2ccc(-c3ccccc3-n3c4ccccc4c4ccccc43)cc2)c2cccc3oc4c5ccccc5ccc4c23)c1 ; c1cc(-c2ccc3sc4ccccc4c3c2)cc(N(c2cccc(-c3cccc4ccccc34)c2)c2cccc3oc4c5ccccc5ccc4c23)c1 ; c1cc(-c2ccc3sc4ccccc4c3c2)cc(N(c2ccccc2-n2c3ccccc3c3ccccc32)c2cccc3oc4c5ccccc5ccc4c23)c1]

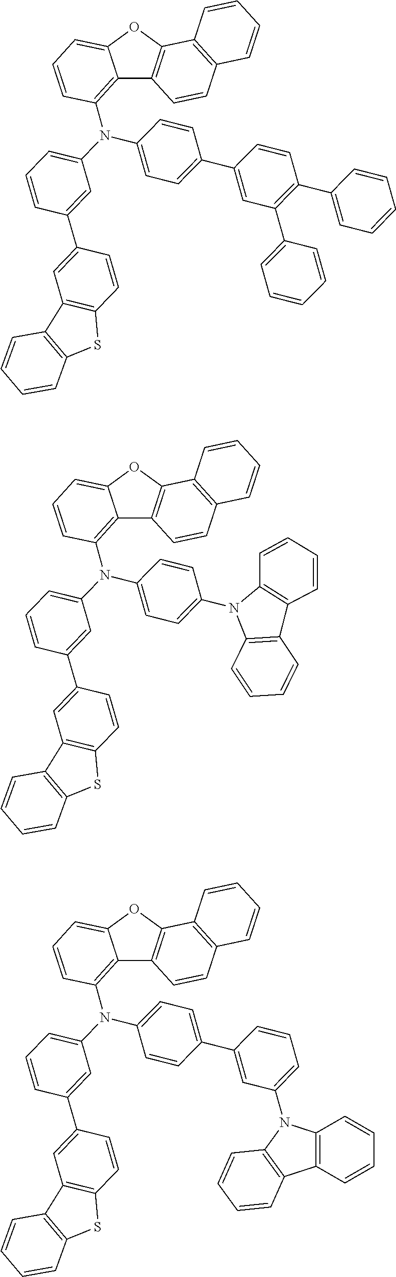 [SMILES: c1ccc(-c2ccc(-c3ccc(N(c4cccc(-c5ccc6sc7ccccc7c6c5)c4)c4cccc5oc6c7ccccc7ccc6c45)cc3)cc2-c2ccccc2)cc1 ; c1cc(-c2ccc3sc4ccccc4c3c2)cc(N(c2ccc(-n3c4ccccc4c4ccccc43)cc2)c2cccc3oc4c5ccccc5ccc4c23)c1 ; c1cc(-c2ccc3sc4ccccc4c3c2)cc(N(c2ccc(-c3cccc(-n4c5ccccc5c5ccccc54)c3)cc2)c2cccc3oc4c5ccccc5ccc4c23)c1]

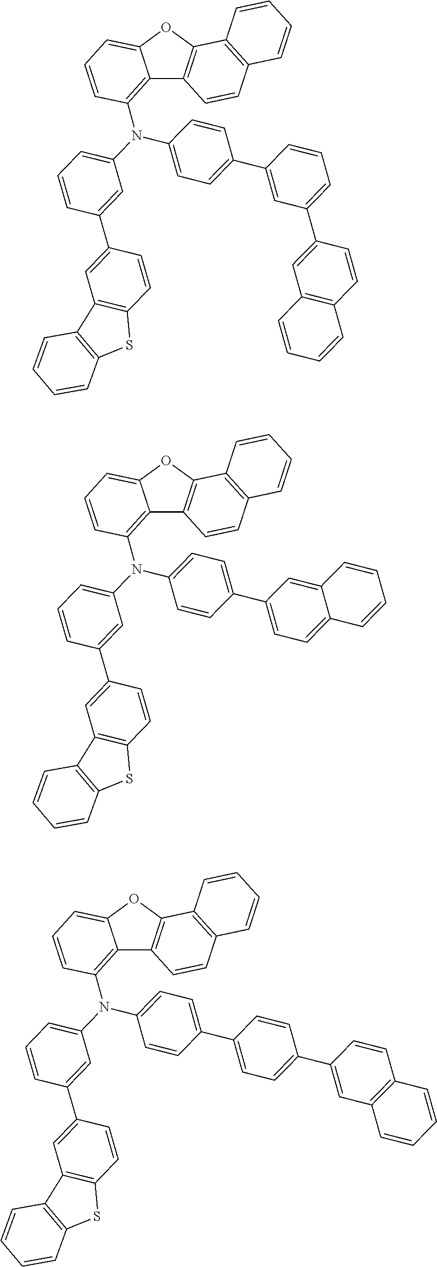 [SMILES: c1cc(-c2ccc(N(c3cccc(-c4ccc5sc6ccccc6c5c4)c3)c3cccc4oc5c6ccccc6ccc5c34)cc2)cc(-c2ccc3ccccc3c2)c1 ; c1cc(-c2ccc3sc4ccccc4c3c2)cc(N(c2ccc(-c3ccc4ccccc4c3)cc2)c2cccc3oc4c5ccccc5ccc4c23)c1 ; c1cc(-c2ccc3sc4ccccc4c3c2)cc(N(c2ccc(-c3ccc(-c4ccc5ccccc5c4)cc3)cc2)c2cccc3oc4c5ccccc5ccc4c23)c1]

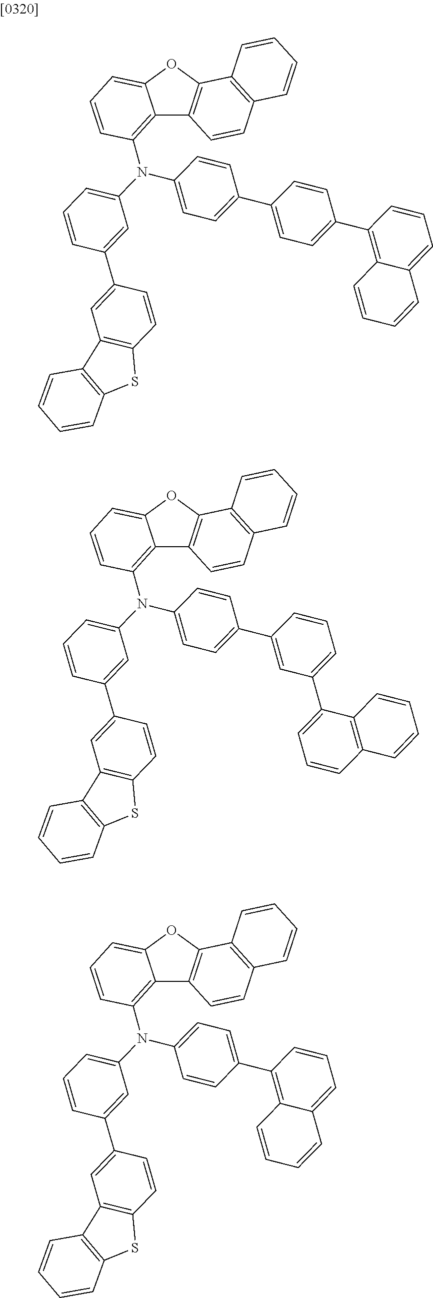 [SMILES: c1cc(-c2ccc3sc4ccccc4c3c2)cc(N(c2ccc(-c3ccc(-c4cccc5ccccc45)cc3)cc2)c2cccc3oc4c5ccccc5ccc4c23)c1 ; c1cc(-c2ccc(N(c3cccc(-c4ccc5sc6ccccc6c5c4)c3)c3cccc4oc5c6ccccc6ccc5c34)cc2)cc(-c2cccc3ccccc23)c1 ; c1cc(-c2ccc3sc4ccccc4c3c2)cc(N(c2ccc(-c3cccc4ccccc34)cc2)c2cccc3oc4c5ccccc5ccc4c23)c1]

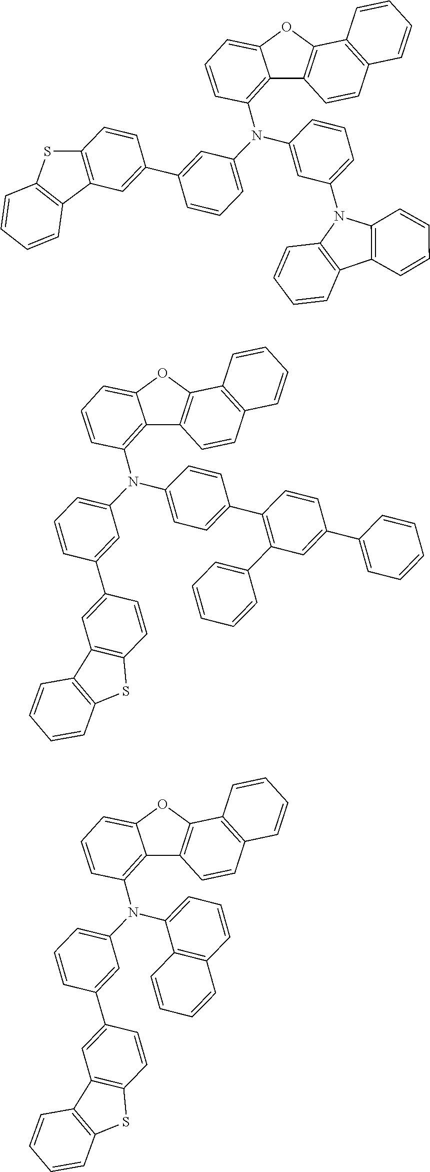 [SMILES: c1cc(-c2ccc3sc4ccccc4c3c2)cc(N(c2cccc(-n3c4ccccc4c4ccccc43)c2)c2cccc3oc4c5ccccc5ccc4c23)c1 ; c1ccc(-c2ccc(-c3ccc(N(c4cccc(-c5ccc6sc7ccccc7c6c5)c4)c4cccc5oc6c7ccccc7ccc6c45)cc3)c(-c3ccccc3)c2)cc1 ; c1cc(-c2ccc3sc4ccccc4c3c2)cc(N(c2cccc3ccccc23)c2cccc3oc4c5ccccc5ccc4c23)c1]

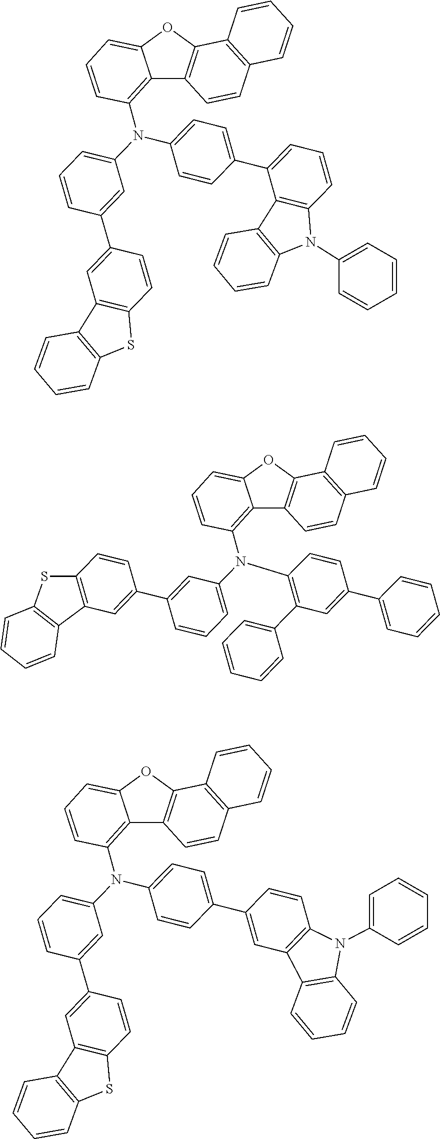 [SMILES: c1ccc(-n2c3ccccc3c3c(-c4ccc(N(c5cccc(-c6ccc7sc8ccccc8c7c6)c5)c5cccc6oc7c8ccccc8ccc7c56)cc4)cccc32)cc1 ; c1ccc(-c2ccc(N(c3cccc(-c4ccc5sc6ccccc6c5c4)c3)c3cccc4oc5c6ccccc6ccc5c34)c(-c3ccccc3)c2)cc1 ; c1ccc(-n2c3ccccc3c3cc(-c4ccc(N(c5cccc(-c6ccc7sc8ccccc8c7c6)c5)c5cccc6oc7c8ccccc8ccc7c56)cc4)ccc32)cc1]

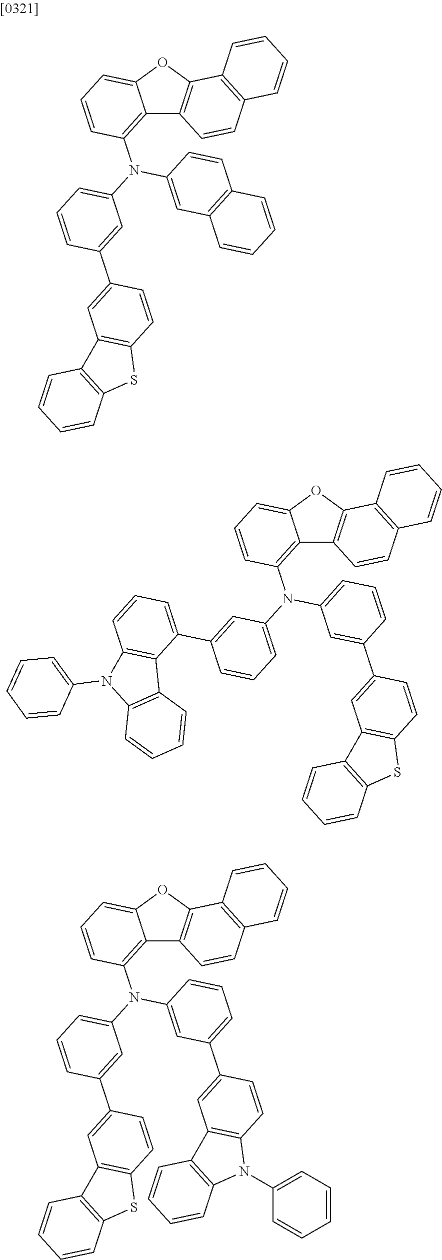 [SMILES: c1cc(-c2ccc3sc4ccccc4c3c2)cc(N(c2ccc3ccccc3c2)c2cccc3oc4c5ccccc5ccc4c23)c1 ; c1ccc(-n2c3ccccc3c3c(-c4cccc(N(c5cccc(-c6ccc7sc8ccccc8c7c6)c5)c5cccc6oc7c8ccccc8ccc7c56)c4)cccc32)cc1 ; c1ccc(-n2c3ccccc3c3cc(-c4cccc(N(c5cccc(-c6ccc7sc8ccccc8c7c6)c5)c5cccc6oc7c8ccccc8ccc7c56)c4)ccc32)cc1]

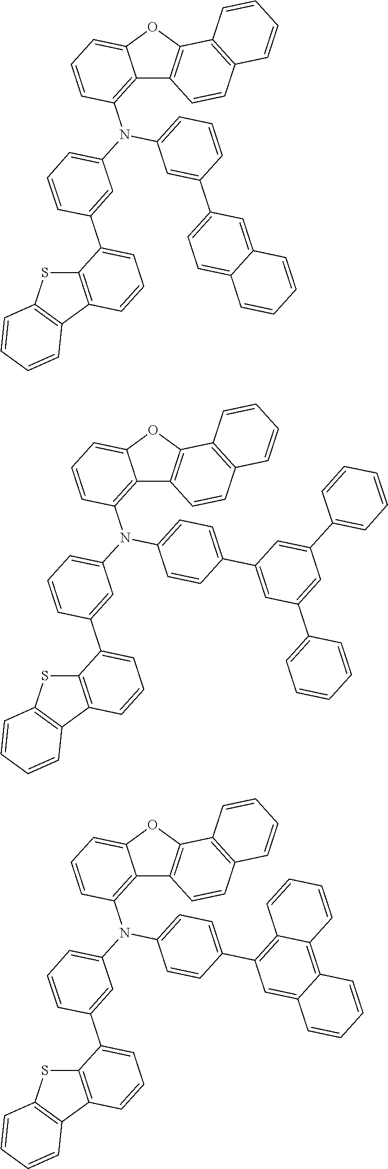 [SMILES: c1cc(-c2ccc3ccccc3c2)cc(N(c2cccc(-c3cccc4c3sc3ccccc34)c2)c2cccc3oc4c5ccccc5ccc4c23)c1 ; c1ccc(-c2cc(-c3ccccc3)cc(-c3ccc(N(c4cccc(-c5cccc6c5sc5ccccc56)c4)c4cccc5oc6c7ccccc7ccc6c45)cc3)c2)cc1 ; c1cc(-c2cccc3c2sc2ccccc23)cc(N(c2ccc(-c3cc4ccccc4c4ccccc34)cc2)c2cccc3oc4c5ccccc5ccc4c23)c1]

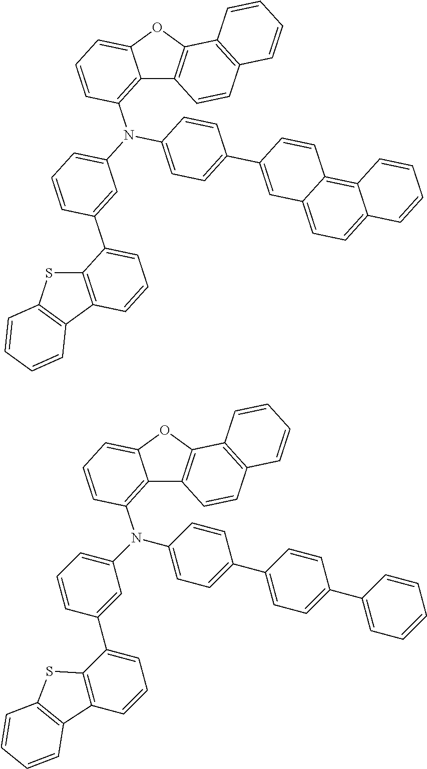 [SMILES: c1cc(-c2cccc3c2sc2ccccc23)cc(N(c2ccc(-c3ccc4c(ccc5ccccc54)c3)cc2)c2cccc3oc4c5ccccc5ccc4c23)c1 ; c1ccc(-c2ccc(-c3ccc(N(c4cccc(-c5cccc6c5sc5ccccc56)c4)c4cccc5oc6c7ccccc7ccc6c45)cc3)cc2)cc1]

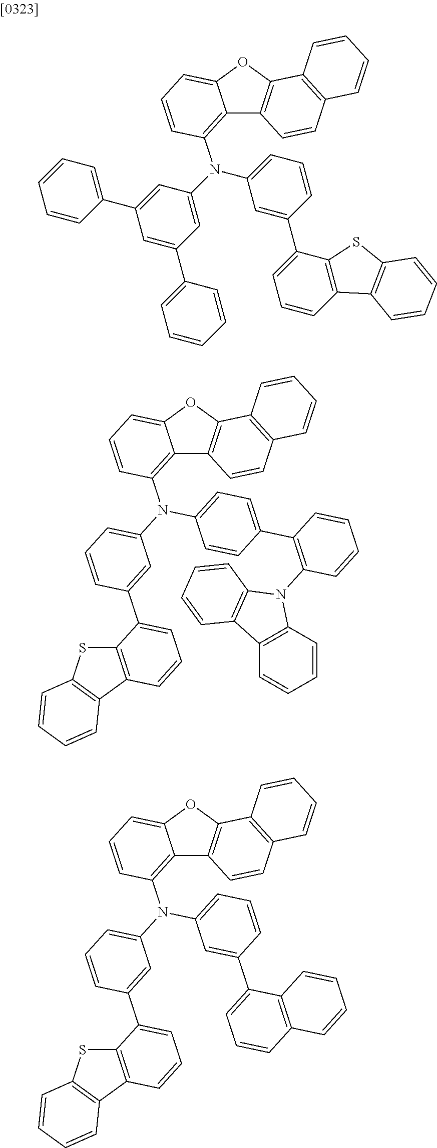 [SMILES: c1ccc(-c2cc(-c3ccccc3)cc(N(c3cccc(-c4cccc5c4sc4ccccc45)c3)c3cccc4oc5c6ccccc6ccc5c34)c2)cc1 ; c1cc(-c2cccc3c2sc2ccccc23)cc(N(c2ccc(-c3ccccc3-n3c4ccccc4c4ccccc43)cc2)c2cccc3oc4c5ccccc5ccc4c23)c1 ; c1cc(-c2cccc3ccccc23)cc(N(c2cccc(-c3cccc4c3sc3ccccc34)c2)c2cccc3oc4c5ccccc5ccc4c23)c1]

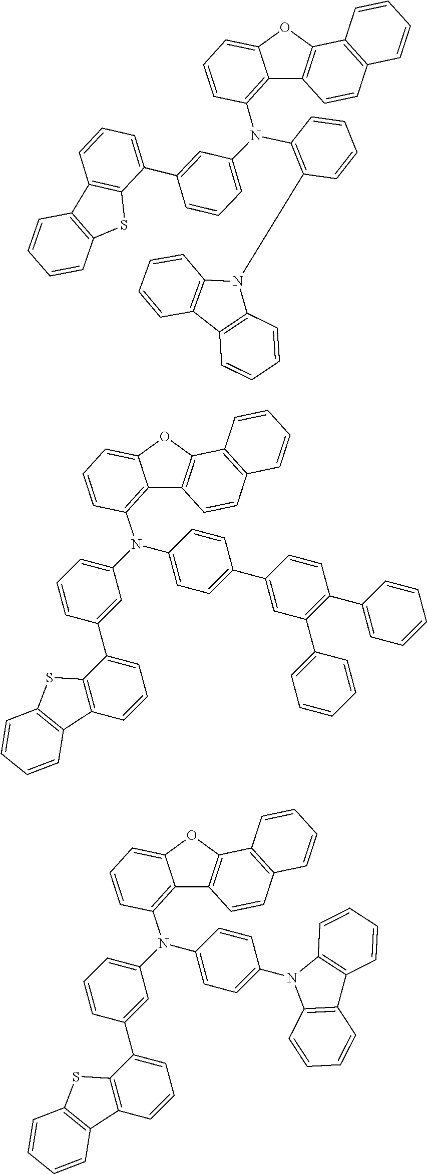

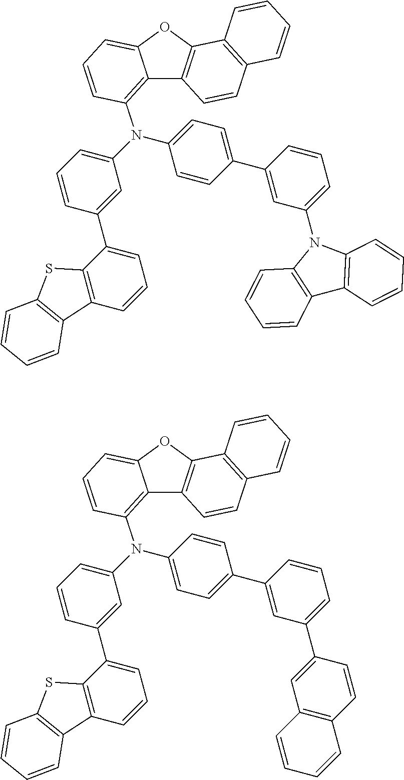 [SMILES: c1cc(-c2cccc3c2sc2ccccc23)cc(N(c2ccc(-c3cccc(-n4c5ccccc5c5ccccc54)c3)cc2)c2cccc3oc4c5ccccc5ccc4c23)c1 ; c1cc(-c2ccc(N(c3cccc(-c4cccc5c4sc4ccccc45)c3)c3cccc4oc5c6ccccc6ccc5c34)cc2)cc(-c2ccc3ccccc3c2)c1]

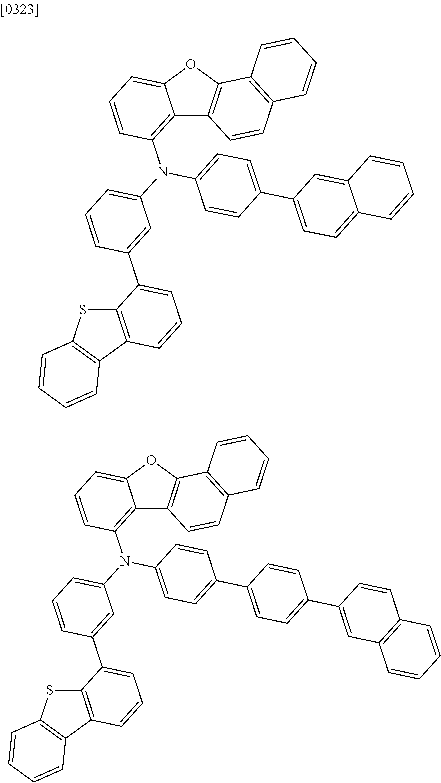 [SMILES: c1cc(-c2cccc3c2sc2ccccc23)cc(N(c2ccc(-c3ccc4ccccc4c3)cc2)c2cccc3oc4c5ccccc5ccc4c23)c1 ; c1cc(-c2cccc3c2sc2ccccc23)cc(N(c2ccc(-c3ccc(-c4ccc5ccccc5c4)cc3)cc2)c2cccc3oc4c5ccccc5ccc4c23)c1]

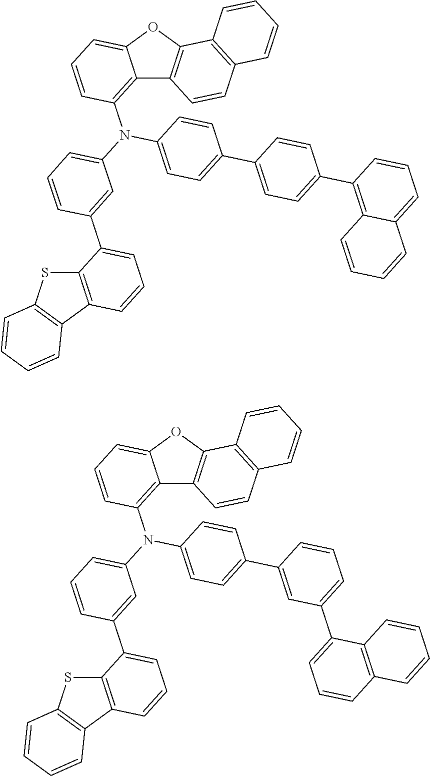 [SMILES: c1cc(-c2cccc3c2sc2ccccc23)cc(N(c2ccc(-c3ccc(-c4cccc5ccccc45)cc3)cc2)c2cccc3oc4c5ccccc5ccc4c23)c1 ; c1cc(-c2ccc(N(c3cccc(-c4cccc5c4sc4ccccc45)c3)c3cccc4oc5c6ccccc6ccc5c34)cc2)cc(-c2cccc3ccccc23)c1]

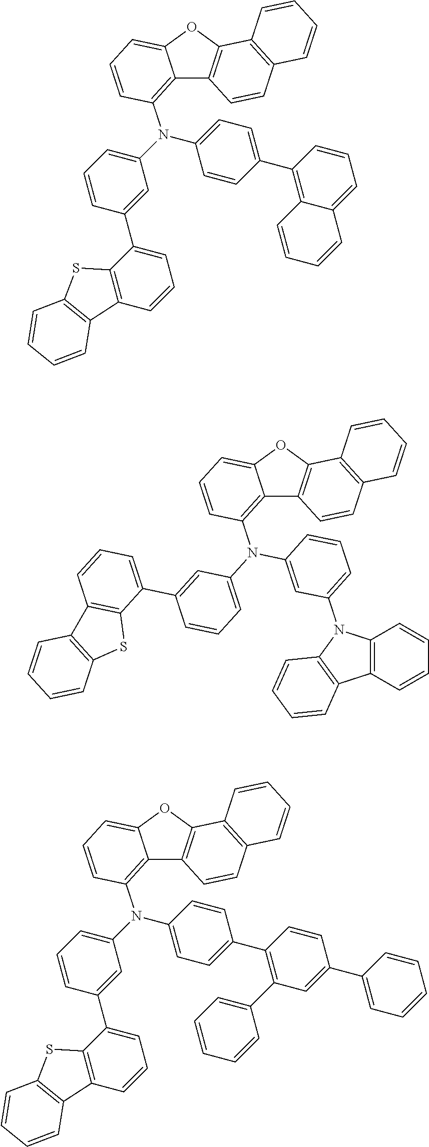 [SMILES: c1cc(-c2cccc3c2sc2ccccc23)cc(N(c2ccc(-c3cccc4ccccc34)cc2)c2cccc3oc4c5ccccc5ccc4c23)c1 ; c1cc(-c2cccc3c2sc2ccccc23)cc(N(c2cccc(-n3c4ccccc4c4ccccc43)c2)c2cccc3oc4c5ccccc5ccc4c23)c1 ; c1ccc(-c2ccc(-c3ccc(N(c4cccc(-c5cccc6c5sc5ccccc56)c4)c4cccc5oc6c7ccccc7ccc6c45)cc3)c(-c3ccccc3)c2)cc1]

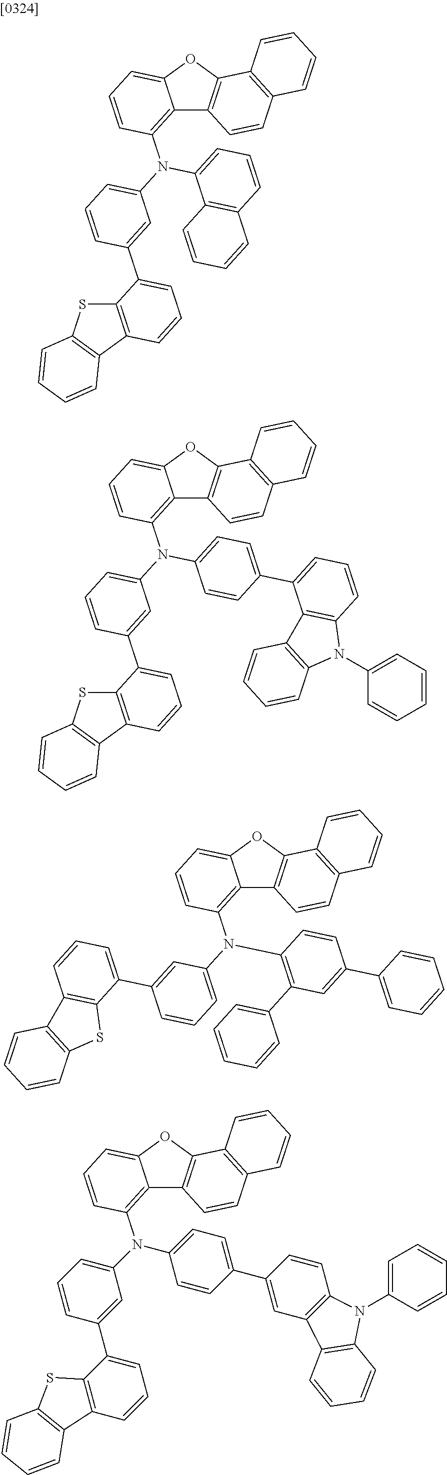

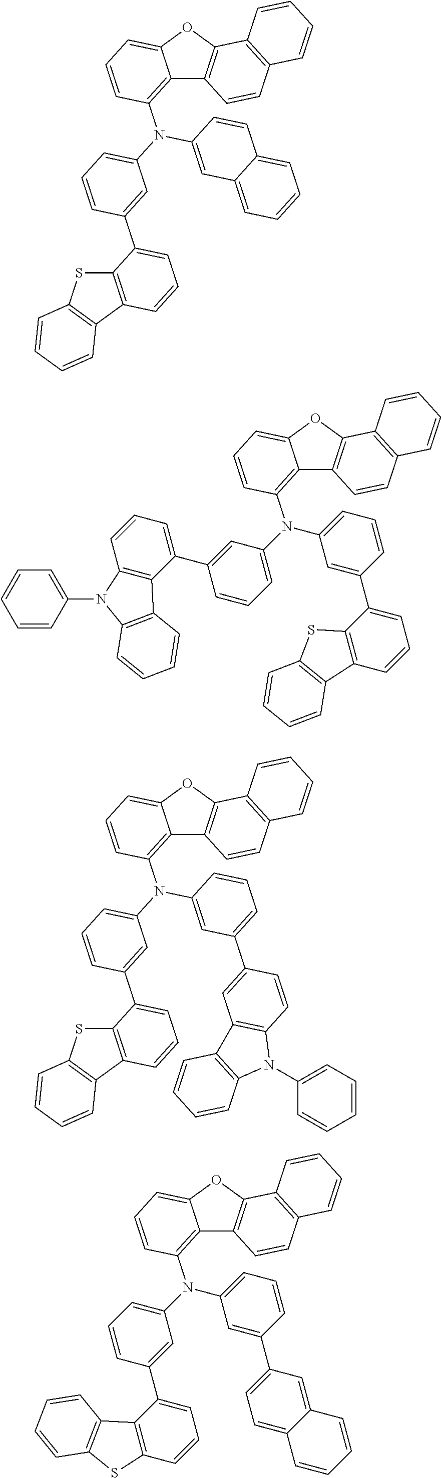

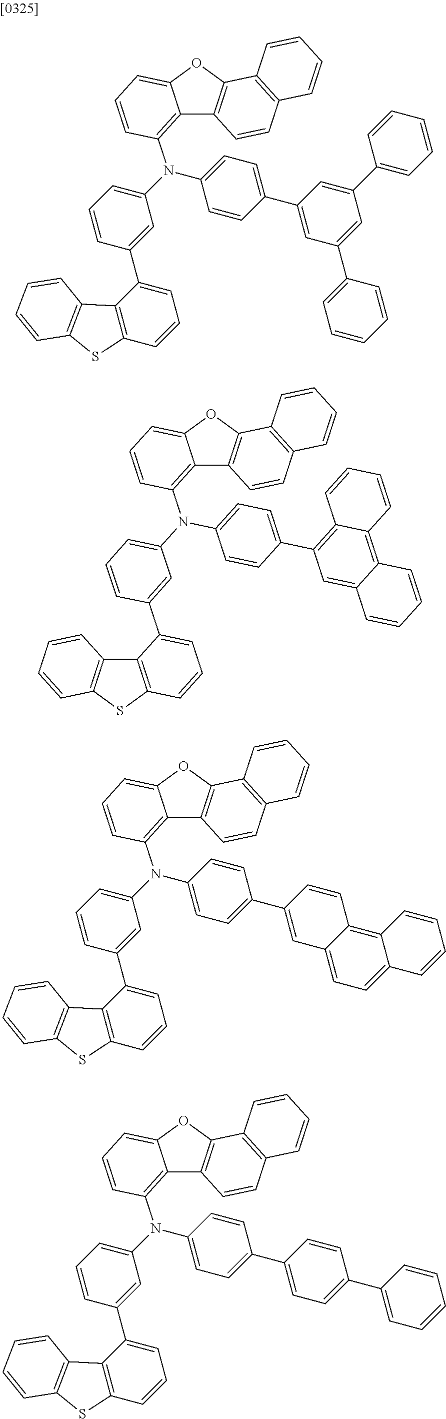 [SMILES: c1ccc(-c2cc(-c3ccccc3)cc(-c3ccc(N(c4cccc(-c5cccc6sc7ccccc7c56)c4)c4cccc5oc6c7ccccc7ccc6c45)cc3)c2)cc1 ; c1cc(-c2cccc3sc4ccccc4c23)cc(N(c2ccc(-c3cc4ccccc4c4ccccc34)cc2)c2cccc3oc4c5ccccc5ccc4c23)c1 ; c1cc(-c2cccc3sc4ccccc4c23)cc(N(c2ccc(-c3ccc4c(ccc5ccccc54)c3)cc2)c2cccc3oc4c5ccccc5ccc4c23)c1 ; c1ccc(-c2ccc(-c3ccc(N(c4cccc(-c5cccc6sc7ccccc7c56)c4)c4cccc5oc6c7ccccc7ccc6c45)cc3)cc2)cc1]

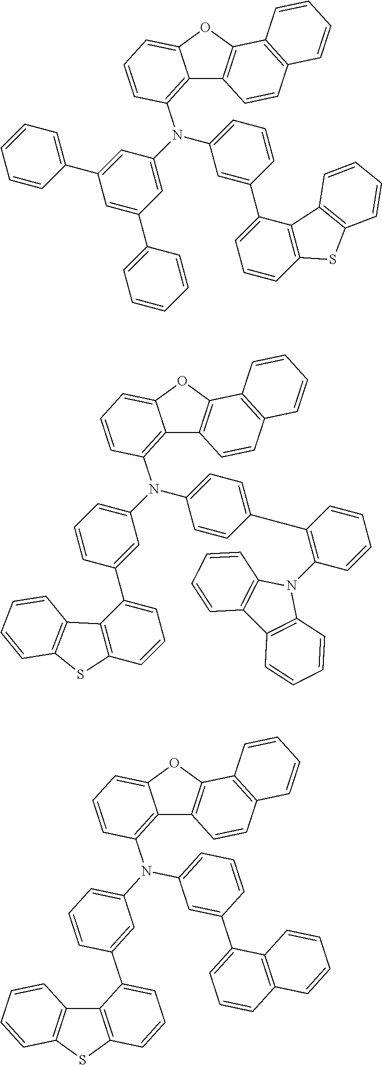 [SMILES: c1ccc(-c2cc(-c3ccccc3)cc(N(c3cccc(-c4cccc5sc6ccccc6c45)c3)c3cccc4oc5c6ccccc6ccc5c34)c2)cc1 ; c1cc(-c2cccc3sc4ccccc4c23)cc(N(c2ccc(-c3ccccc3-n3c4ccccc4c4ccccc43)cc2)c2cccc3oc4c5ccccc5ccc4c23)c1 ; c1cc(-c2cccc3ccccc23)cc(N(c2cccc(-c3cccc4sc5ccccc5c34)c2)c2cccc3oc4c5ccccc5ccc4c23)c1]

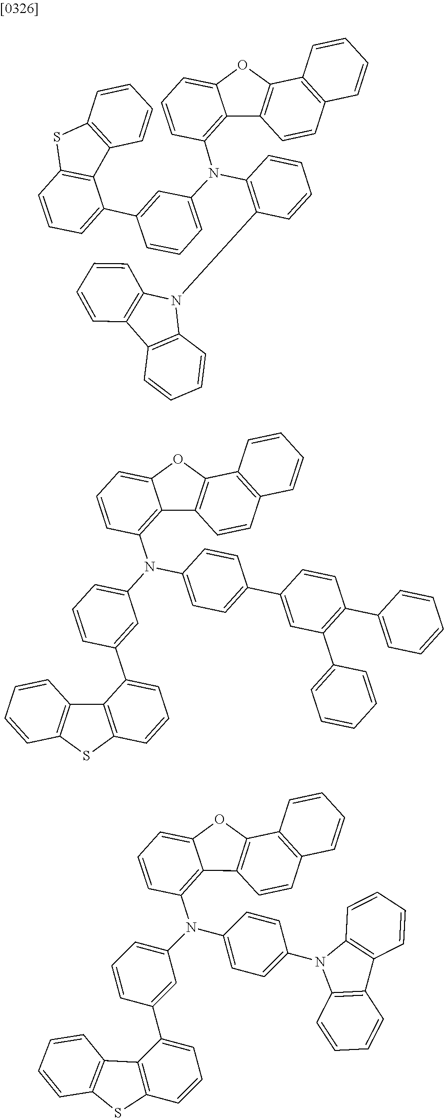 [SMILES: c1cc(-c2cccc3sc4ccccc4c23)cc(N(c2ccccc2-n2c3ccccc3c3ccccc32)c2cccc3oc4c5ccccc5ccc4c23)c1 ; c1ccc(-c2ccc(-c3ccc(N(c4cccc(-c5cccc6sc7ccccc7c56)c4)c4cccc5oc6c7ccccc7ccc6c45)cc3)cc2-c2ccccc2)cc1 ; c1cc(-c2cccc3sc4ccccc4c23)cc(N(c2ccc(-n3c4ccccc4c4ccccc43)cc2)c2cccc3oc4c5ccccc5ccc4c23)c1]

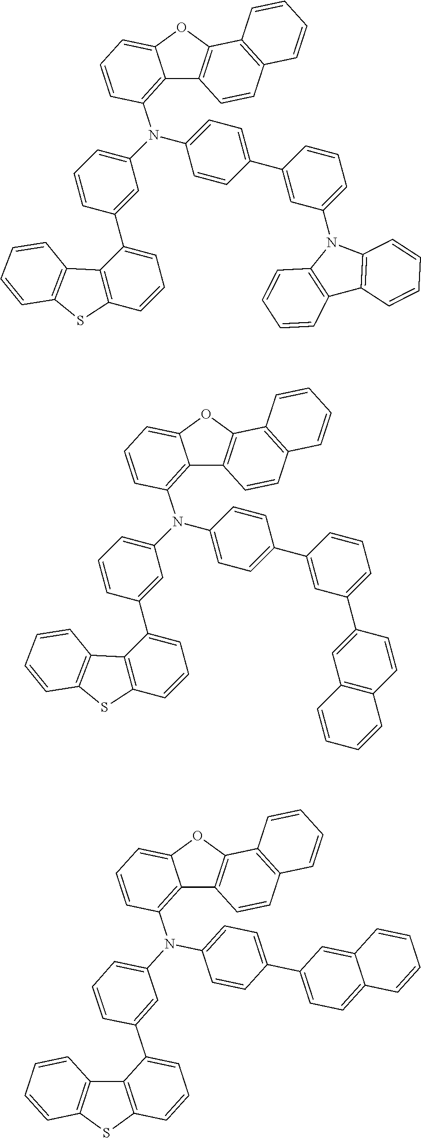 [SMILES: c1cc(-c2cccc3sc4ccccc4c23)cc(N(c2ccc(-c3cccc(-n4c5ccccc5c5ccccc54)c3)cc2)c2cccc3oc4c5ccccc5ccc4c23)c1 ; c1cc(-c2ccc(N(c3cccc(-c4cccc5sc6ccccc6c45)c3)c3cccc4oc5c6ccccc6ccc5c34)cc2)cc(-c2ccc3ccccc3c2)c1 ; c1cc(-c2cccc3sc4ccccc4c23)cc(N(c2ccc(-c3ccc4ccccc4c3)cc2)c2cccc3oc4c5ccccc5ccc4c23)c1]

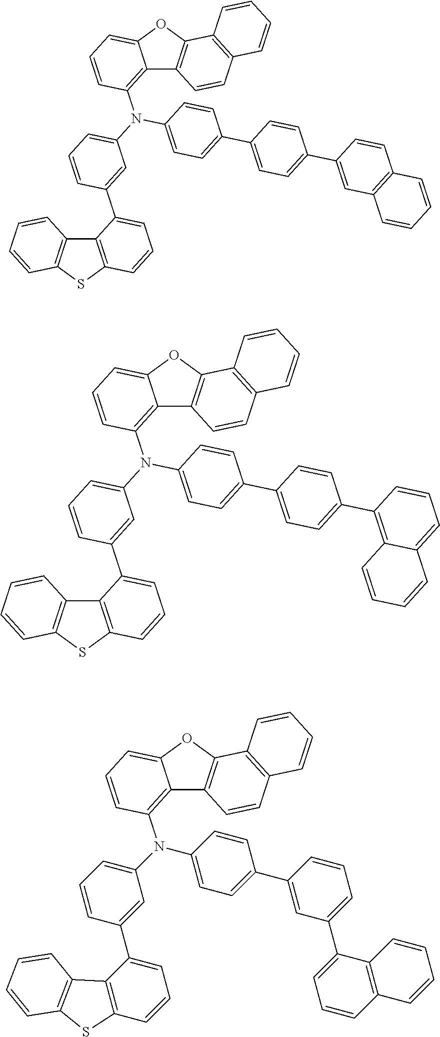 [SMILES: c1cc(-c2cccc3sc4ccccc4c23)cc(N(c2ccc(-c3ccc(-c4ccc5ccccc5c4)cc3)cc2)c2cccc3oc4c5ccccc5ccc4c23)c1 ; c1cc(-c2cccc3sc4ccccc4c23)cc(N(c2ccc(-c3ccc(-c4cccc5ccccc45)cc3)cc2)c2cccc3oc4c5ccccc5ccc4c23)c1 ; c1cc(-c2ccc(N(c3cccc(-c4cccc5sc6ccccc6c45)c3)c3cccc4oc5c6ccccc6ccc5c34)cc2)cc(-c2cccc3ccccc23)c1]

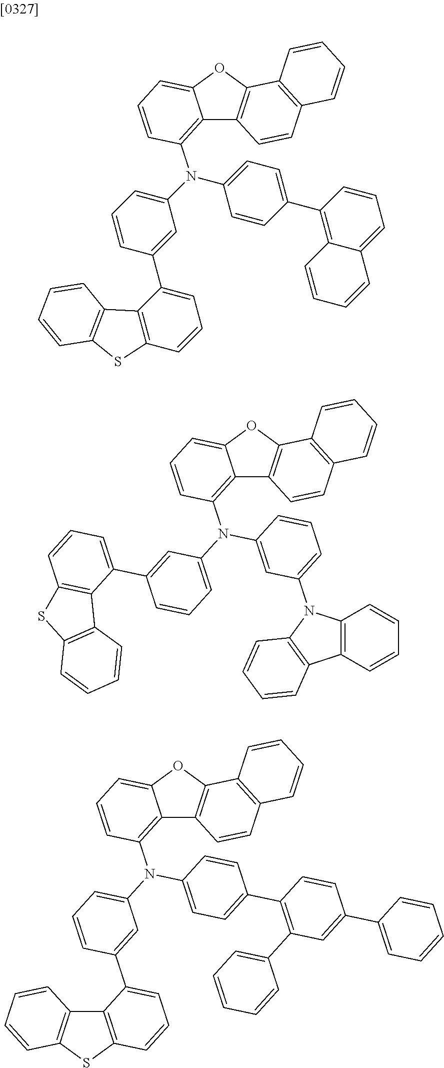

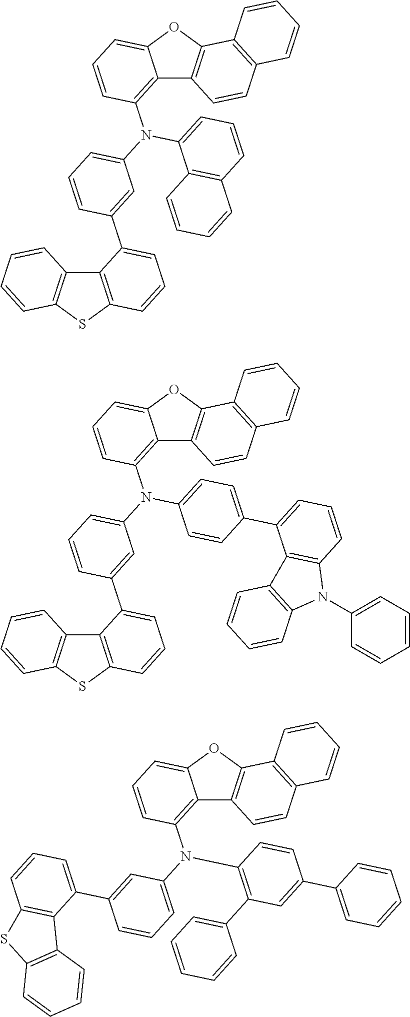 [SMILES: c1cc(-c2cccc3sc4ccccc4c23)cc(N(c2cccc3ccccc23)c2cccc3oc4c5ccccc5ccc4c23)c1 ; c1ccc(-n2c3ccccc3c3c(-c4ccc(N(c5cccc(-c6cccc7sc8ccccc8c67)c5)c5cccc6oc7c8ccccc8ccc7c56)cc4)cccc32)cc1 ; c1ccc(-c2ccc(N(c3cccc(-c4cccc5sc6ccccc6c45)c3)c3cccc4oc5c6ccccc6ccc5c34)c(-c3ccccc3)c2)cc1]

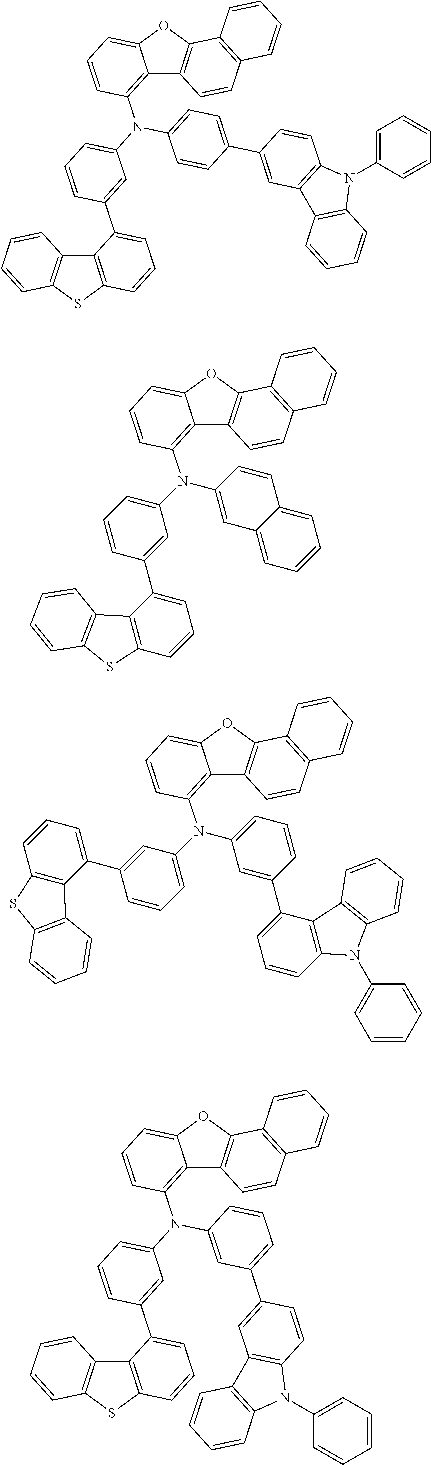 [SMILES: c1ccc(-n2c3ccccc3c3cc(-c4ccc(N(c5cccc(-c6cccc7sc8ccccc8c67)c5)c5cccc6oc7c8ccccc8ccc7c56)cc4)ccc32)cc1 ; c1cc(-c2cccc3sc4ccccc4c23)cc(N(c2ccc3ccccc3c2)c2cccc3oc4c5ccccc5ccc4c23)c1 ; c1ccc(-n2c3ccccc3c3c(-c4cccc(N(c5cccc(-c6cccc7sc8ccccc8c67)c5)c5cccc6oc7c8ccccc8ccc7c56)c4)cccc32)cc1 ; c1ccc(-n2c3ccccc3c3cc(-c4cccc(N(c5cccc(-c6cccc7sc8ccccc8c67)c5)c5cccc6oc7c8ccccc8ccc7c56)c4)ccc32)cc1]

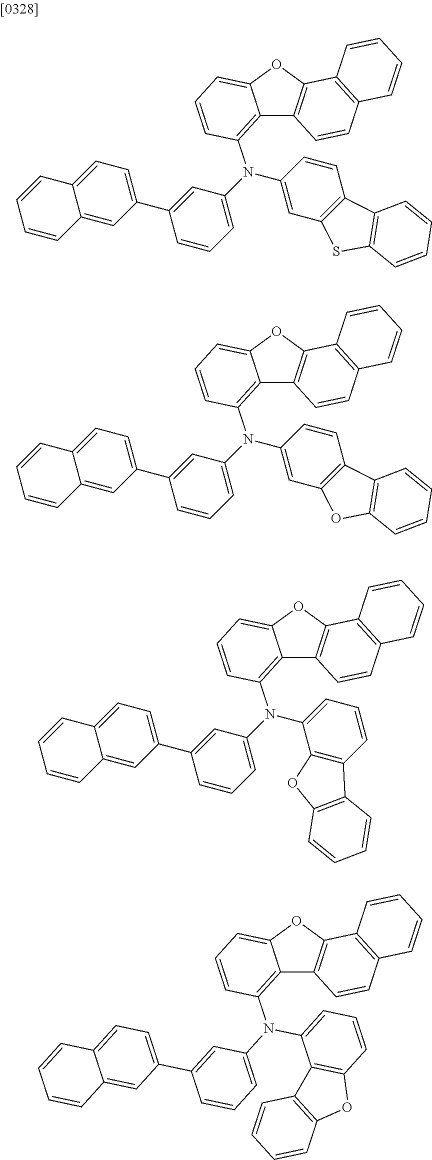

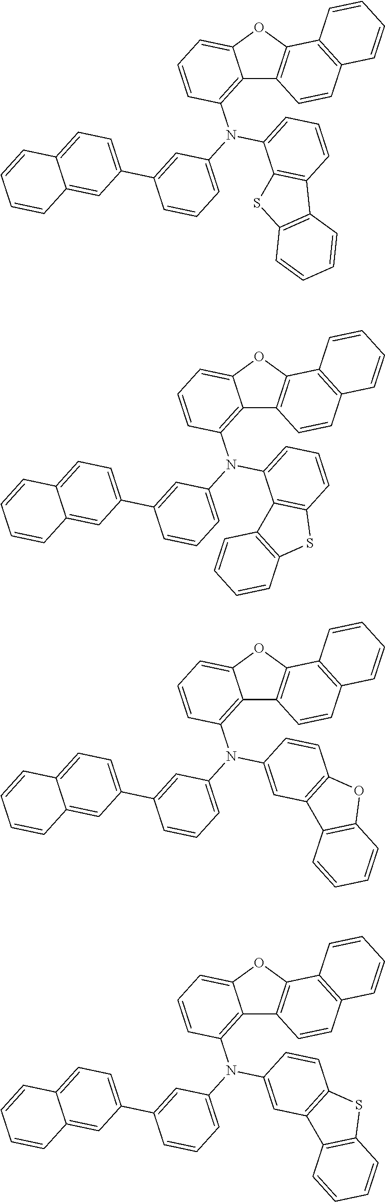 [SMILES: c1cc(-c2ccc3ccccc3c2)cc(N(c2cccc3c2sc2ccccc23)c2cccc3oc4c5ccccc5ccc4c23)c1 ; c1cc(-c2ccc3ccccc3c2)cc(N(c2cccc3oc4c5ccccc5ccc4c23)c2cccc3sc4ccccc4c23)c1 ; c1cc(-c2ccc3ccccc3c2)cc(N(c2ccc3oc4ccccc4c3c2)c2cccc3oc4c5ccccc5ccc4c23)c1 ; c1cc(-c2ccc3ccccc3c2)cc(N(c2ccc3sc4ccccc4c3c2)c2cccc3oc4c5ccccc5ccc4c23)c1]

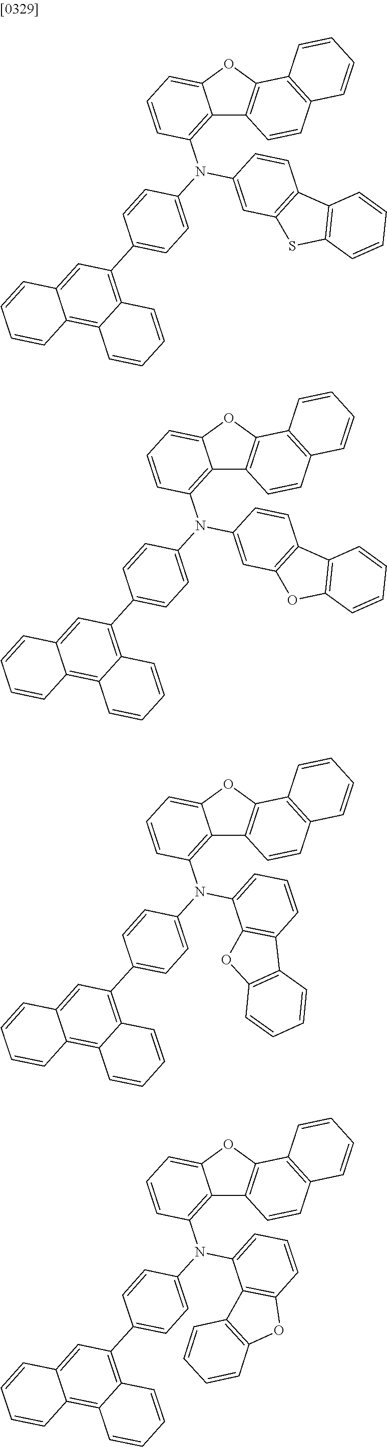 [SMILES: c1ccc2c(c1)cc(-c1ccc(N(c3ccc4c(c3)sc3ccccc34)c3cccc4oc5c6ccccc6ccc5c34)cc1)c1ccccc12 ; c1ccc2c(c1)cc(-c1ccc(N(c3ccc4c(c3)oc3ccccc34)c3cccc4oc5c6ccccc6ccc5c34)cc1)c1ccccc12 ; c1ccc2c(c1)cc(-c1ccc(N(c3cccc4c3oc3ccccc34)c3cccc4oc5c6ccccc6ccc5c34)cc1)c1ccccc12 ; c1ccc2c(c1)cc(-c1ccc(N(c3cccc4oc5ccccc5c34)c3cccc4oc5c6ccccc6ccc5c34)cc1)c1ccccc12]

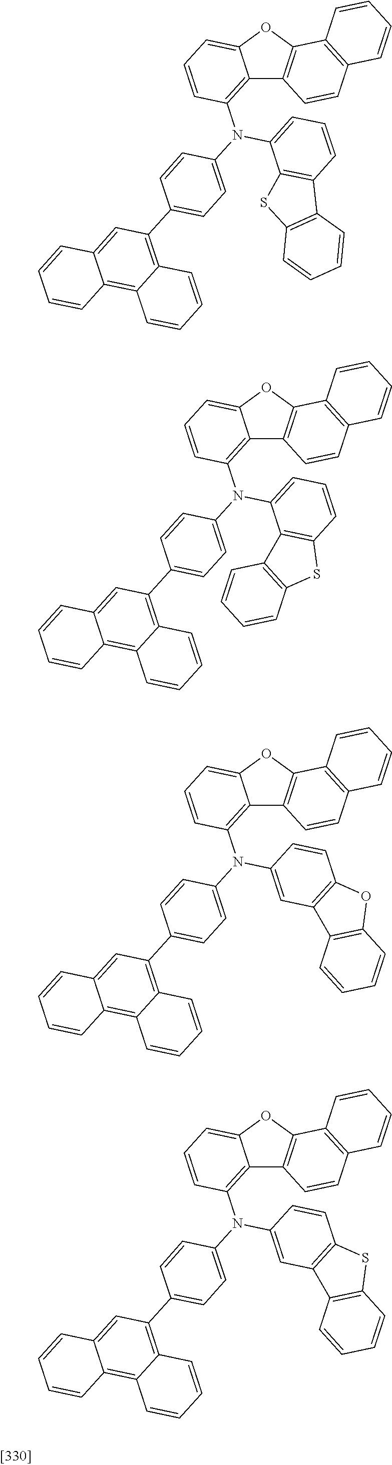 [SMILES: c1ccc2c(c1)cc(-c1ccc(N(c3cccc4c3sc3ccccc34)c3cccc4oc5c6ccccc6ccc5c34)cc1)c1ccccc12 ; c1ccc2c(c1)cc(-c1ccc(N(c3cccc4oc5c6ccccc6ccc5c34)c3cccc4sc5ccccc5c34)cc1)c1ccccc12 ; c1ccc2c(c1)cc(-c1ccc(N(c3ccc4oc5ccccc5c4c3)c3cccc4oc5c6ccccc6ccc5c34)cc1)c1ccccc12 ; c1ccc2c(c1)cc(-c1ccc(N(c3ccc4sc5ccccc5c4c3)c3cccc4oc5c6ccccc6ccc5c34)cc1)c1ccccc12]

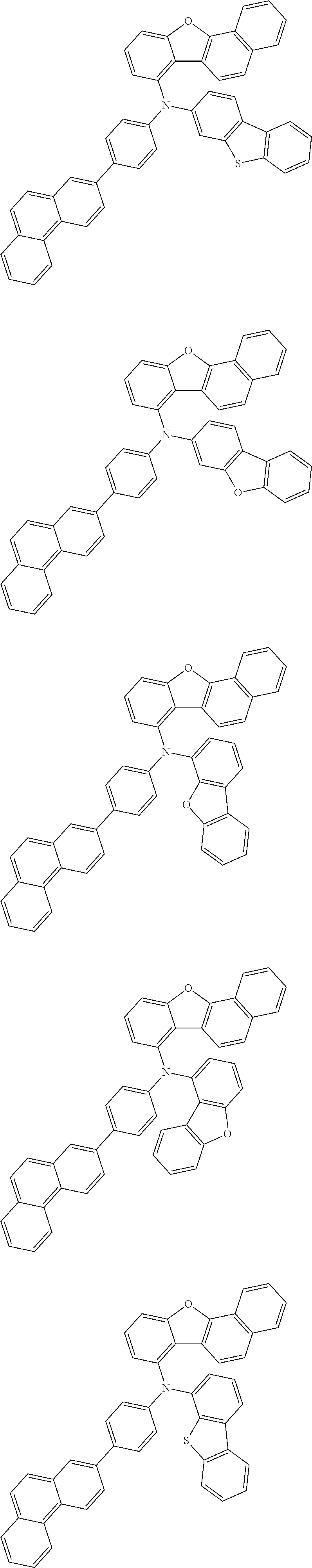 [SMILES: c1ccc2c(c1)ccc1cc(-c3ccc(N(c4ccc5c(c4)sc4ccccc45)c4cccc5oc6c7ccccc7ccc6c45)cc3)ccc12 ; c1ccc2c(c1)ccc1cc(-c3ccc(N(c4ccc5c(c4)oc4ccccc45)c4cccc5oc6c7ccccc7ccc6c45)cc3)ccc12 ; c1ccc2c(c1)ccc1cc(-c3ccc(N(c4cccc5c4oc4ccccc45)c4cccc5oc6c7ccccc7ccc6c45)cc3)ccc12 ; c1ccc2c(c1)ccc1cc(-c3ccc(N(c4cccc5oc6ccccc6c45)c4cccc5oc6c7ccccc7ccc6c45)cc3)ccc12 ; c1ccc2c(c1)ccc1cc(-c3ccc(N(c4cccc5c4sc4ccccc45)c4cccc5oc6c7ccccc7ccc6c45)cc3)ccc12]

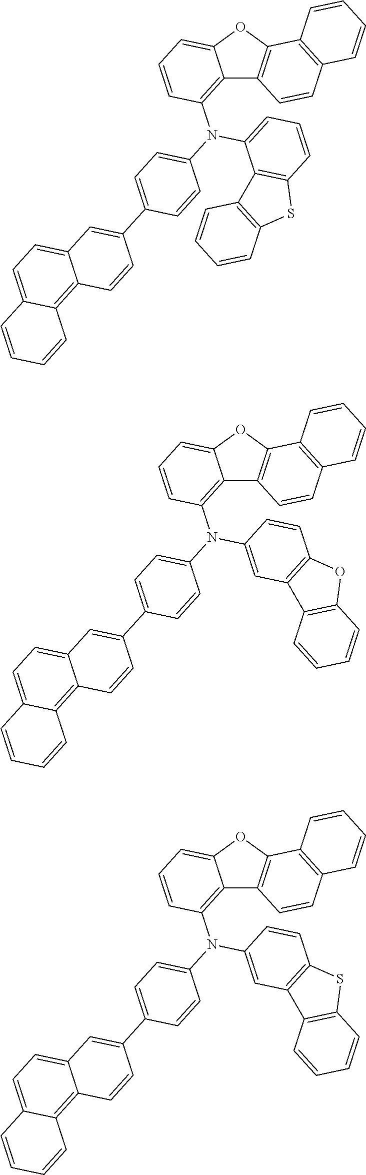 [SMILES: c1ccc2c(c1)ccc1cc(-c3ccc(N(c4cccc5oc6c7ccccc7ccc6c45)c4cccc5sc6ccccc6c45)cc3)ccc12 ; c1ccc2c(c1)ccc1cc(-c3ccc(N(c4ccc5oc6ccccc6c5c4)c4cccc5oc6c7ccccc7ccc6c45)cc3)ccc12 ; c1ccc2c(c1)ccc1cc(-c3ccc(N(c4ccc5sc6ccccc6c5c4)c4cccc5oc6c7ccccc7ccc6c45)cc3)ccc12]

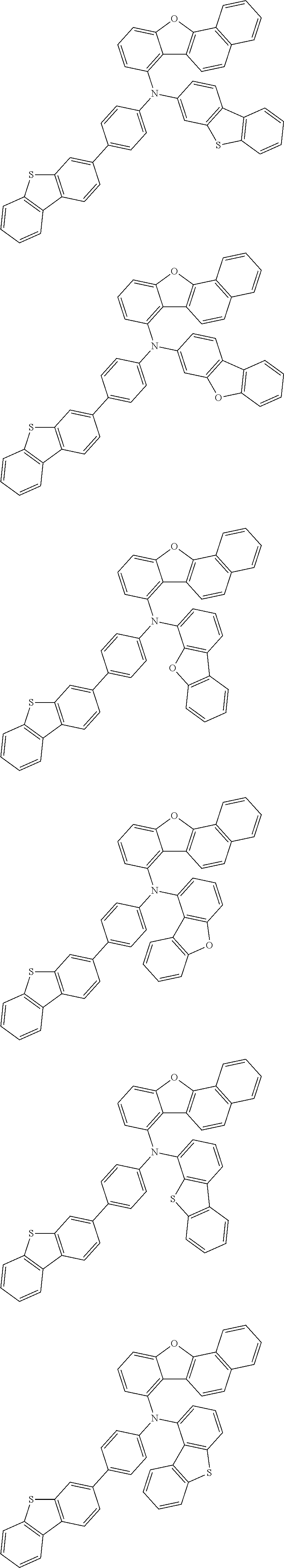

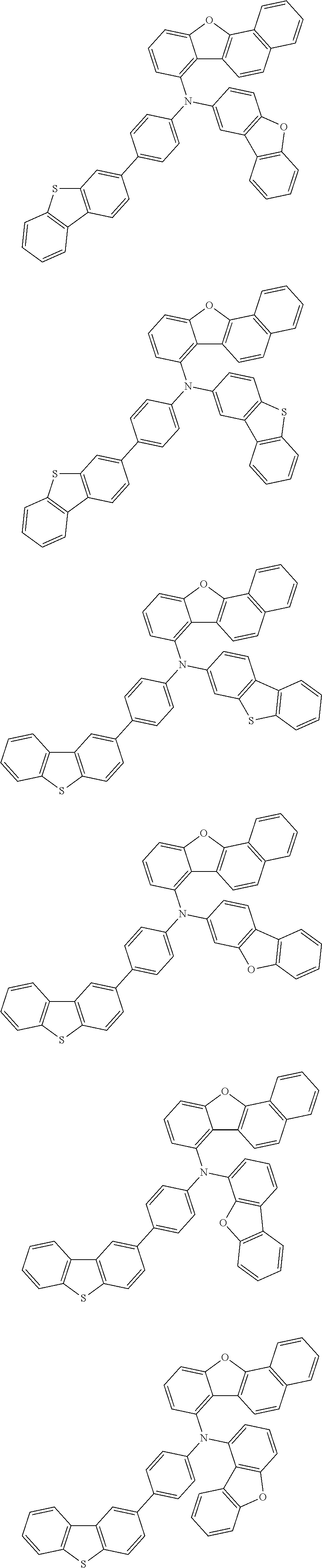

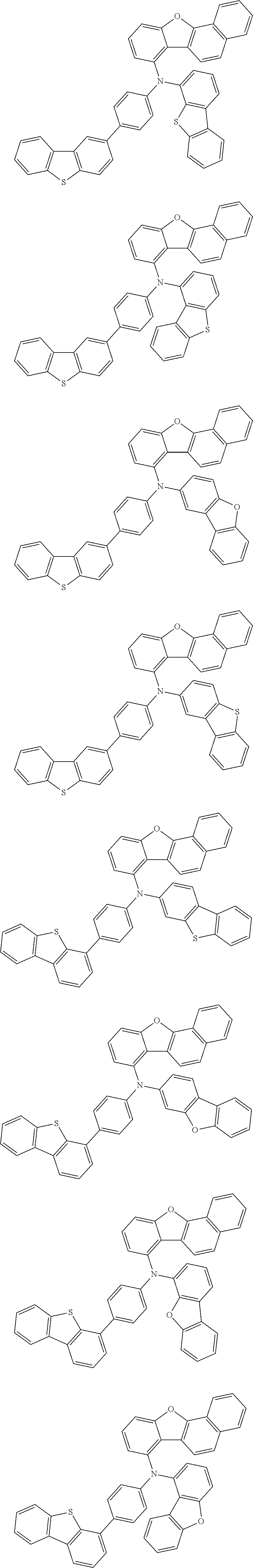

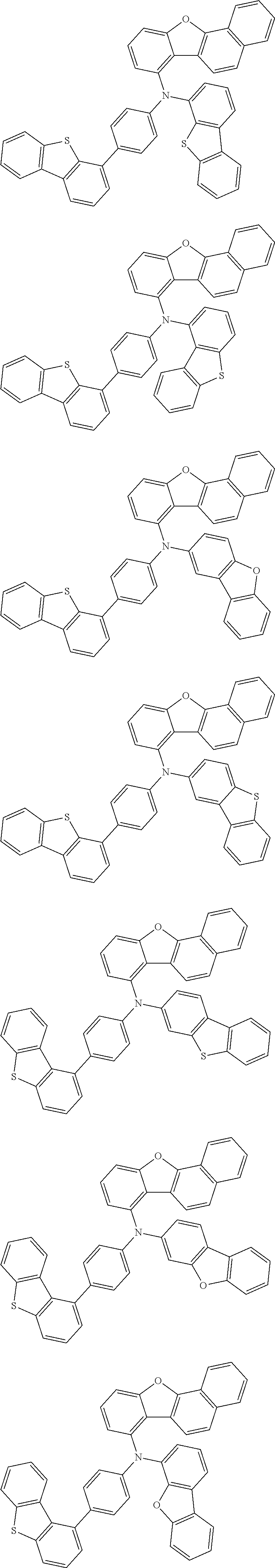

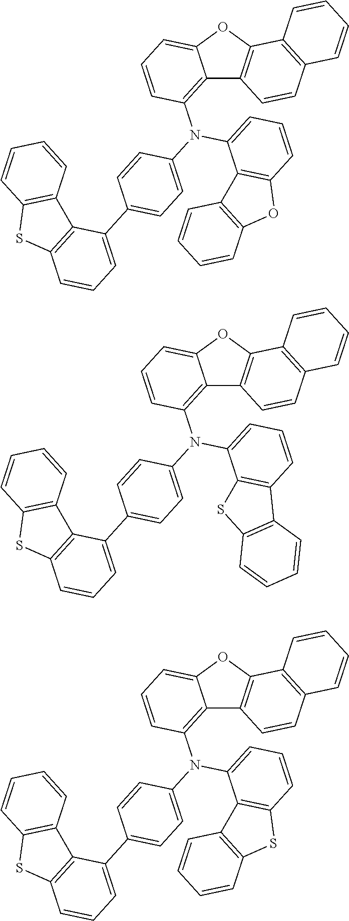 [SMILES: c1ccc2c(c1)ccc1c2oc2cccc(N(c3ccc(-c4cccc5sc6ccccc6c45)cc3)c3cccc4oc5ccccc5c34)c21 ; c1ccc2c(c1)ccc1c2oc2cccc(N(c3ccc(-c4cccc5sc6ccccc6c45)cc3)c3cccc4c3sc3ccccc34)c21 ; c1ccc2c(c1)ccc1c2oc2cccc(N(c3ccc(-c4cccc5sc6ccccc6c45)cc3)c3cccc4sc5ccccc5c34)c21]

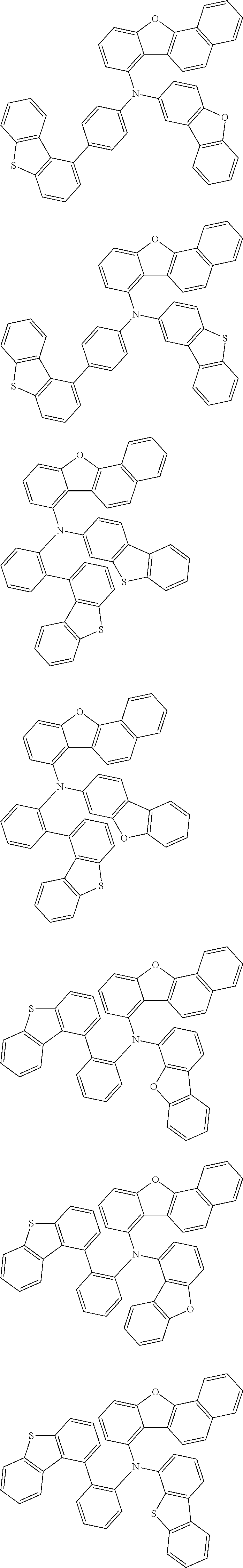

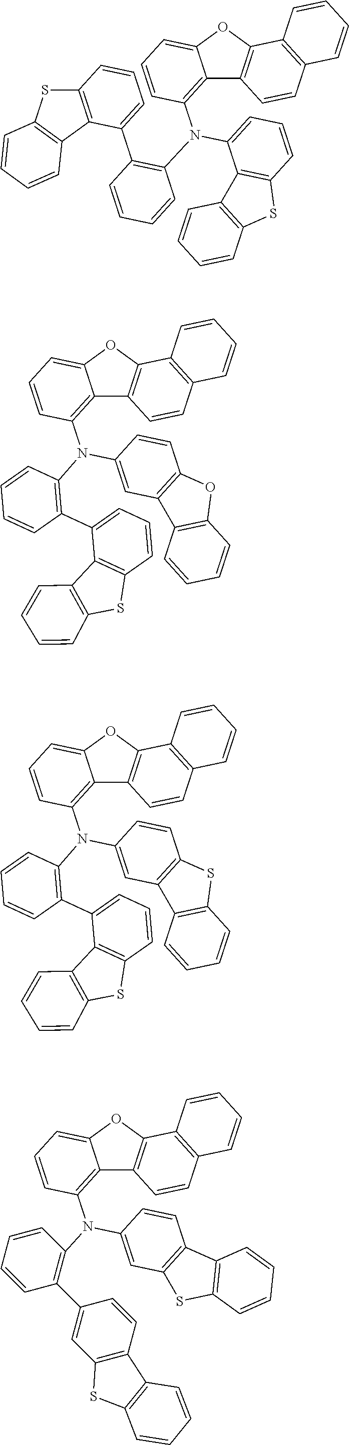 [SMILES: c1ccc(N(c2cccc3oc4c5ccccc5ccc4c23)c2cccc3sc4ccccc4c23)c(-c2cccc3sc4ccccc4c23)c1 ; c1ccc(N(c2ccc3oc4ccccc4c3c2)c2cccc3oc4c5ccccc5ccc4c23)c(-c2cccc3sc4ccccc4c23)c1 ; c1ccc(N(c2ccc3sc4ccccc4c3c2)c2cccc3oc4c5ccccc5ccc4c23)c(-c2cccc3sc4ccccc4c23)c1 ; c1ccc(N(c2ccc3c(c2)sc2ccccc23)c2cccc3oc4c5ccccc5ccc4c23)c(-c2ccc3c(c2)sc2ccccc23)c1]

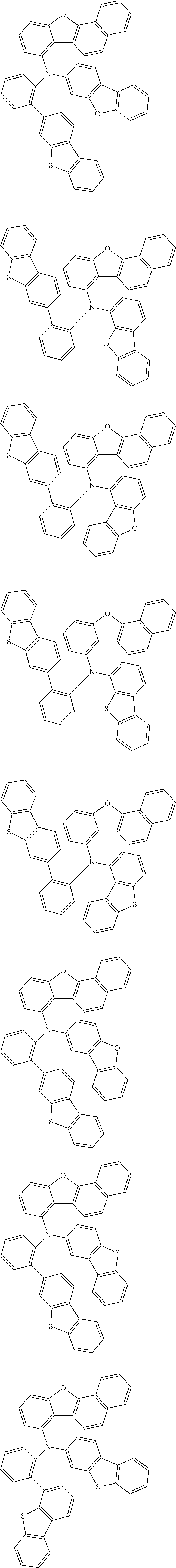

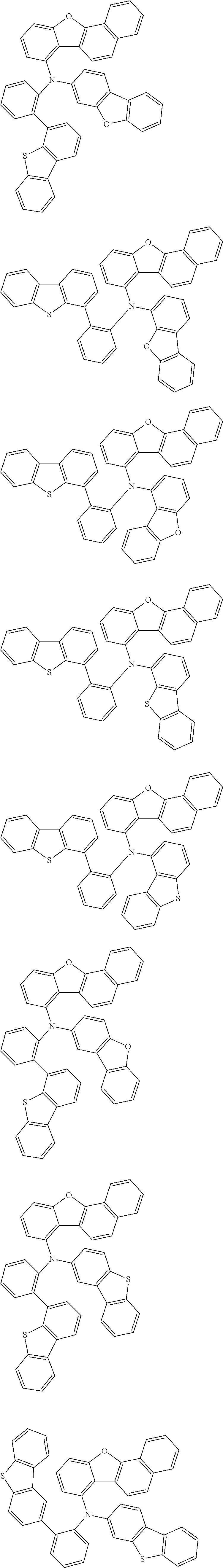

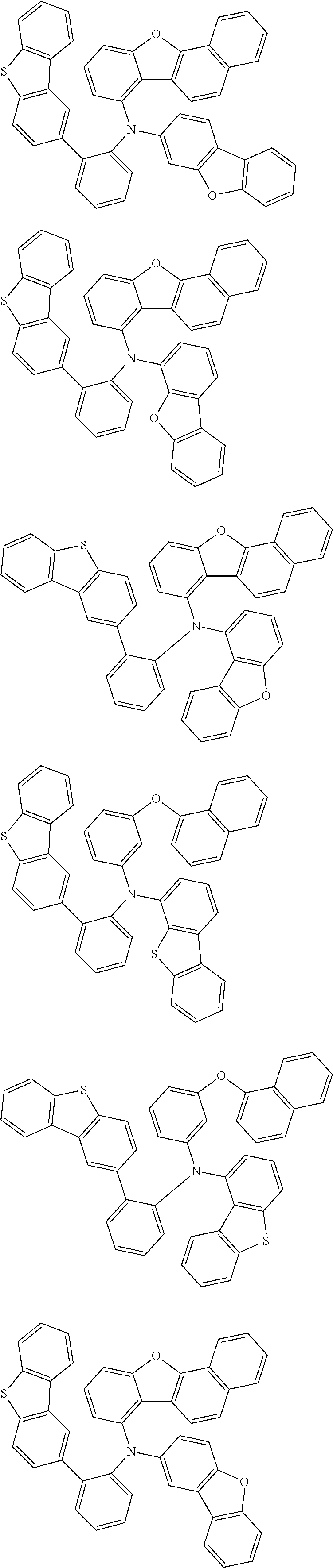

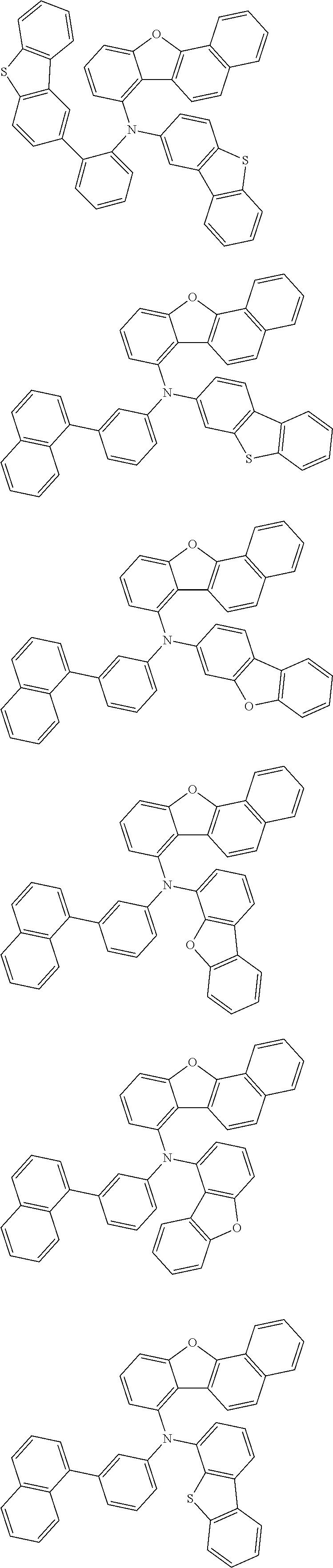

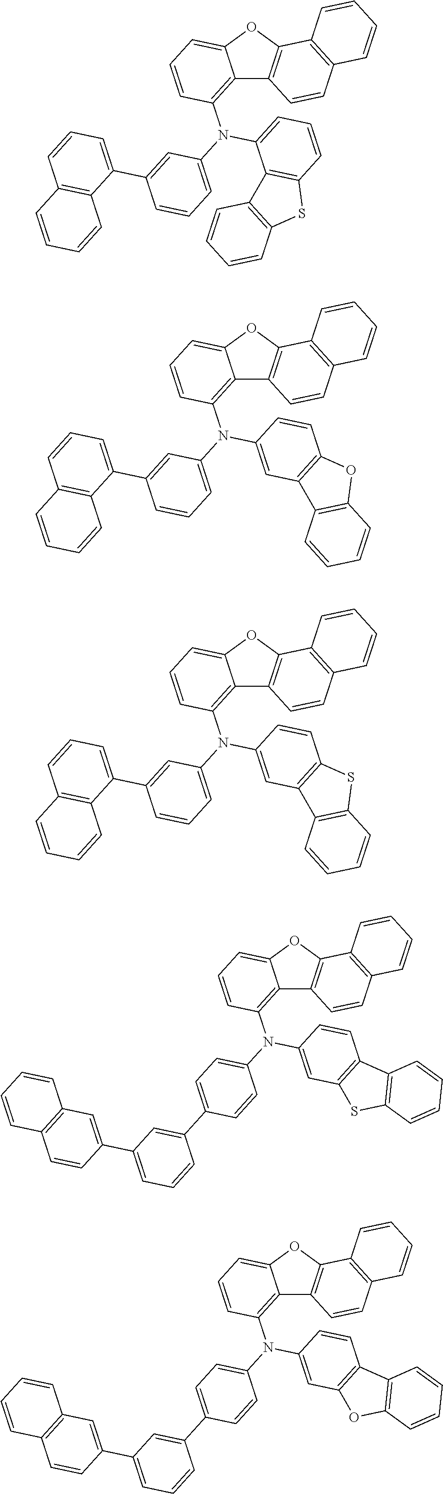 [SMILES: c1cc(-c2cccc3ccccc23)cc(N(c2cccc3oc4c5ccccc5ccc4c23)c2cccc3sc4ccccc4c23)c1 ; c1cc(-c2cccc3ccccc23)cc(N(c2ccc3oc4ccccc4c3c2)c2cccc3oc4c5ccccc5ccc4c23)c1 ; c1cc(-c2cccc3ccccc23)cc(N(c2ccc3sc4ccccc4c3c2)c2cccc3oc4c5ccccc5ccc4c23)c1 ; c1cc(-c2ccc(N(c3ccc4c(c3)sc3ccccc34)c3cccc4oc5c6ccccc6ccc5c34)cc2)cc(-c2ccc3ccccc3c2)c1 ; c1cc(-c2ccc(N(c3ccc4c(c3)oc3ccccc34)c3cccc4oc5c6ccccc6ccc5c34)cc2)cc(-c2ccc3ccccc3c2)c1]

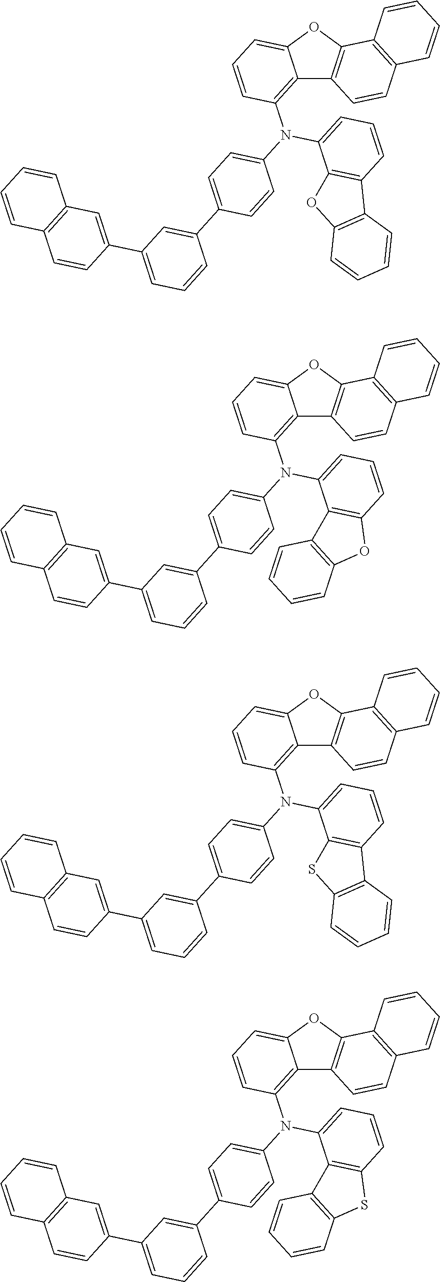 [SMILES: c1cc(-c2ccc(N(c3cccc4c3oc3ccccc34)c3cccc4oc5c6ccccc6ccc5c34)cc2)cc(-c2ccc3ccccc3c2)c1 ; c1cc(-c2ccc(N(c3cccc4oc5ccccc5c34)c3cccc4oc5c6ccccc6ccc5c34)cc2)cc(-c2ccc3ccccc3c2)c1 ; c1cc(-c2ccc(N(c3cccc4c3sc3ccccc34)c3cccc4oc5c6ccccc6ccc5c34)cc2)cc(-c2ccc3ccccc3c2)c1 ; c1cc(-c2ccc(N(c3cccc4oc5c6ccccc6ccc5c34)c3cccc4sc5ccccc5c34)cc2)cc(-c2ccc3ccccc3c2)c1]

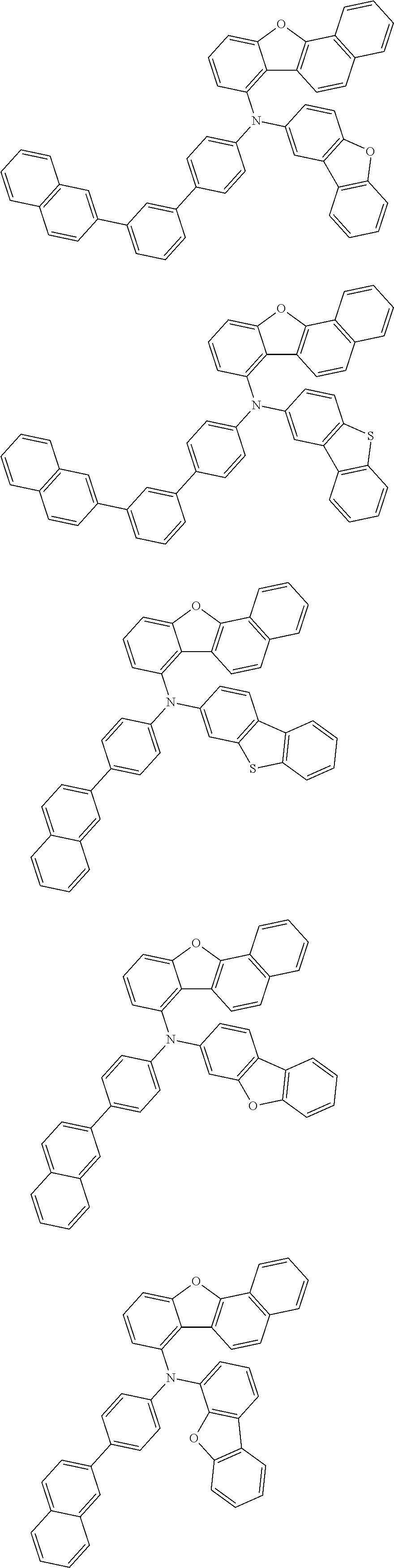 [SMILES: c1cc(-c2ccc(N(c3ccc4oc5ccccc5c4c3)c3cccc4oc5c6ccccc6ccc5c34)cc2)cc(-c2ccc3ccccc3c2)c1 ; c1cc(-c2ccc(N(c3ccc4sc5ccccc5c4c3)c3cccc4oc5c6ccccc6ccc5c34)cc2)cc(-c2ccc3ccccc3c2)c1 ; c1ccc2cc(-c3ccc(N(c4ccc5c(c4)sc4ccccc45)c4cccc5oc6c7ccccc7ccc6c45)cc3)ccc2c1 ; c1ccc2cc(-c3ccc(N(c4ccc5c(c4)oc4ccccc45)c4cccc5oc6c7ccccc7ccc6c45)cc3)ccc2c1 ; c1ccc2cc(-c3ccc(N(c4cccc5c4oc4ccccc45)c4cccc5oc6c7ccccc7ccc6c45)cc3)ccc2c1]

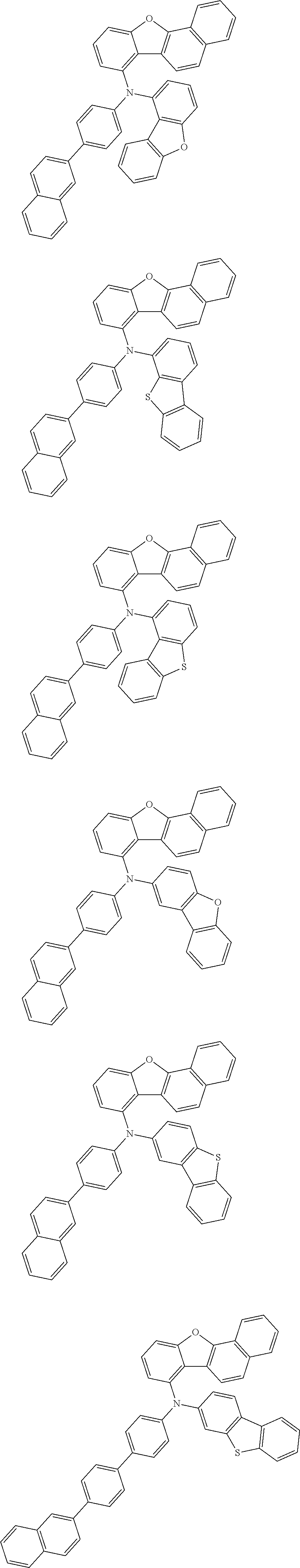 [SMILES: c1ccc2cc(-c3ccc(N(c4cccc5oc6ccccc6c45)c4cccc5oc6c7ccccc7ccc6c45)cc3)ccc2c1 ; c1ccc2cc(-c3ccc(N(c4cccc5c4sc4ccccc45)c4cccc5oc6c7ccccc7ccc6c45)cc3)ccc2c1 ; c1ccc2cc(-c3ccc(N(c4cccc5oc6c7ccccc7ccc6c45)c4cccc5sc6ccccc6c45)cc3)ccc2c1 ; c1ccc2cc(-c3ccc(N(c4ccc5oc6ccccc6c5c4)c4cccc5oc6c7ccccc7ccc6c45)cc3)ccc2c1 ; c1ccc2cc(-c3ccc(N(c4ccc5sc6ccccc6c5c4)c4cccc5oc6c7ccccc7ccc6c45)cc3)ccc2c1 ; c1ccc2cc(-c3ccc(-c4ccc(N(c5ccc6c(c5)sc5ccccc56)c5cccc6oc7c8ccccc8ccc7c56)cc4)cc3)ccc2c1]

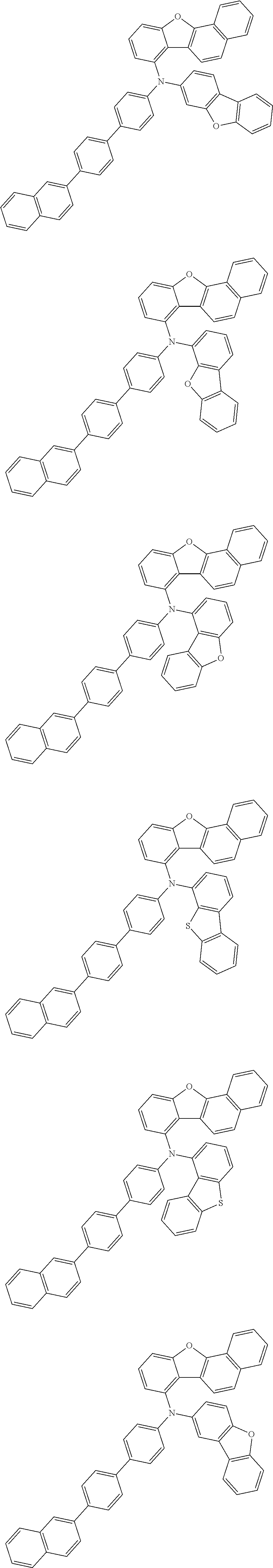

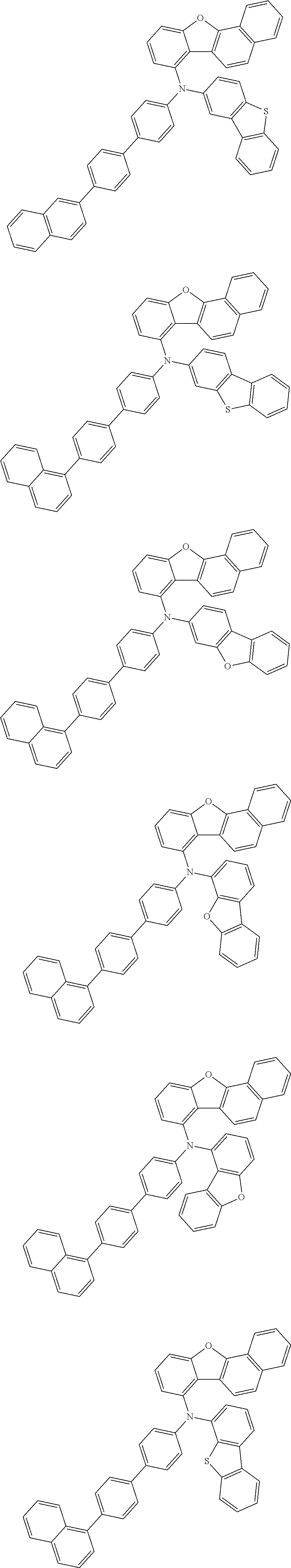

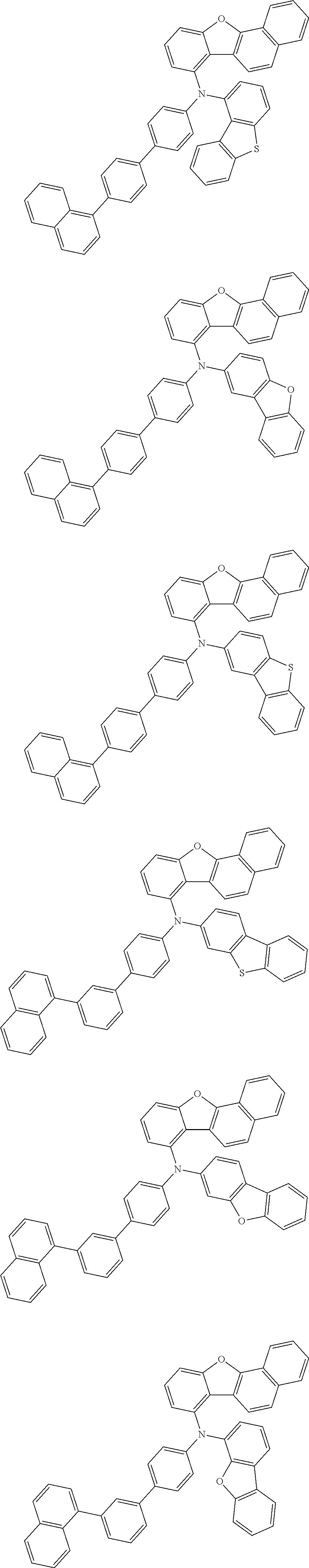

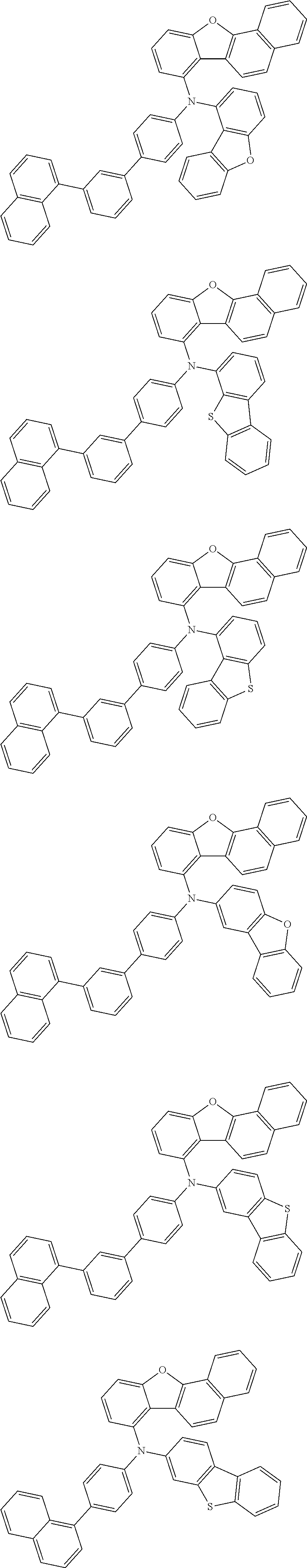

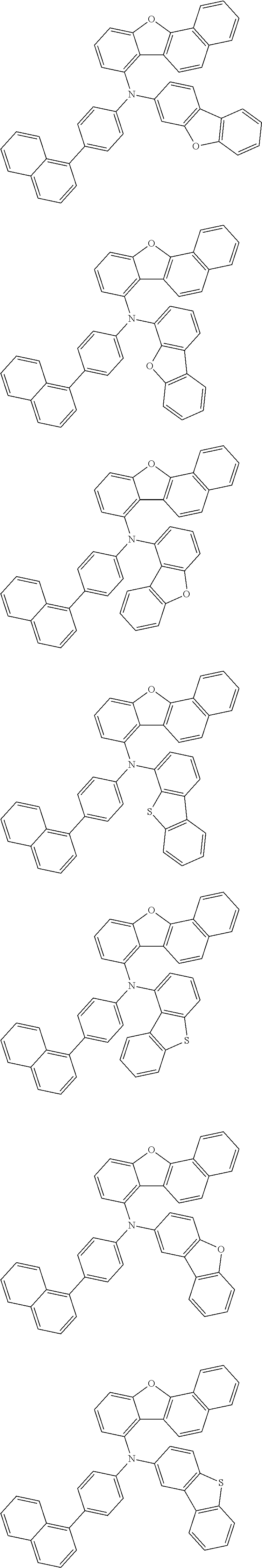

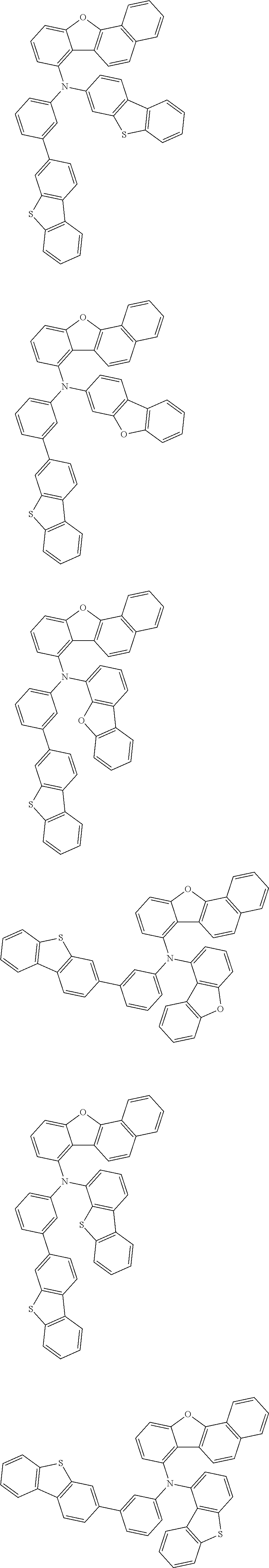

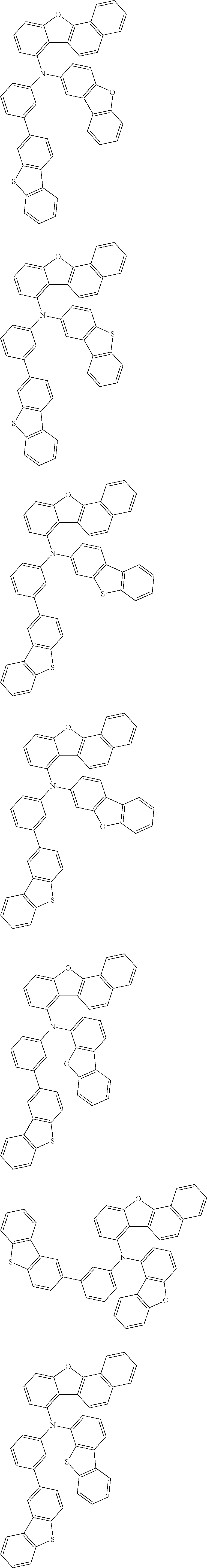

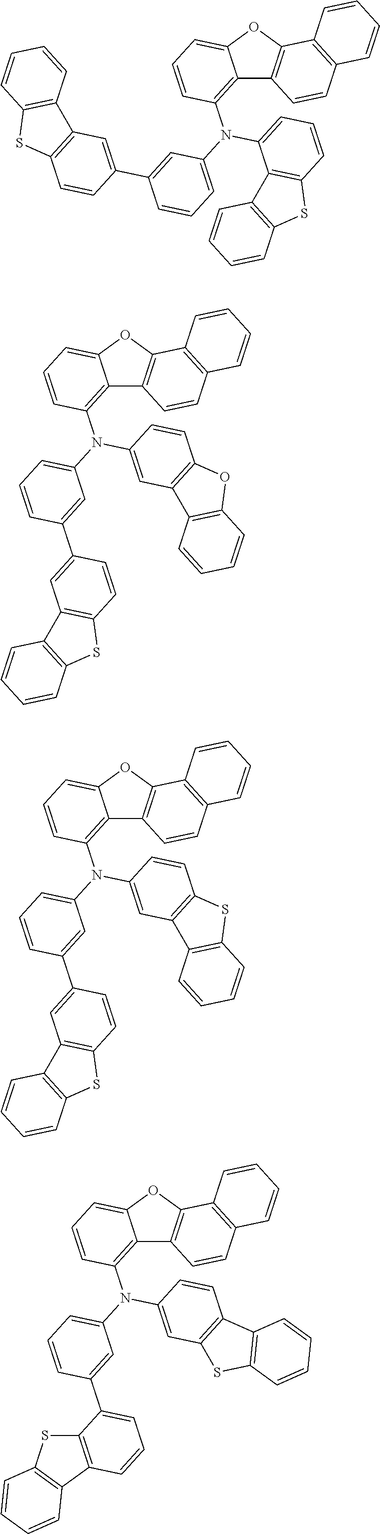

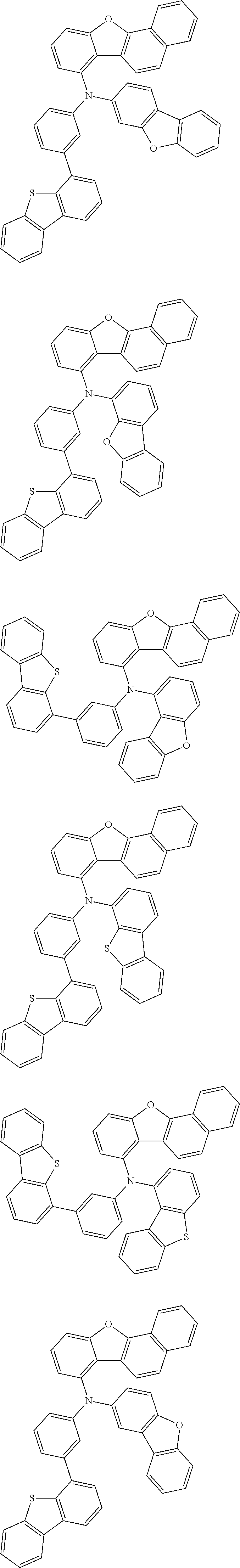 [SMILES: c1cc(-c2cccc3c2sc2ccccc23)cc(N(c2ccc3c(c2)oc2ccccc23)c2cccc3oc4c5ccccc5ccc4c23)c1 ; c1cc(-c2cccc3c2sc2ccccc23)cc(N(c2cccc3c2oc2ccccc23)c2cccc3oc4c5ccccc5ccc4c23)c1 ; c1cc(-c2cccc3c2sc2ccccc23)cc(N(c2cccc3oc4ccccc4c23)c2cccc3oc4c5ccccc5ccc4c23)c1 ; c1cc(-c2cccc3c2sc2ccccc23)cc(N(c2cccc3c2sc2ccccc23)c2cccc3oc4c5ccccc5ccc4c23)c1 ; c1cc(-c2cccc3c2sc2ccccc23)cc(N(c2cccc3oc4c5ccccc5ccc4c23)c2cccc3sc4ccccc4c23)c1 ; c1cc(-c2cccc3c2sc2ccccc23)cc(N(c2ccc3oc4ccccc4c3c2)c2cccc3oc4c5ccccc5ccc4c23)c1]

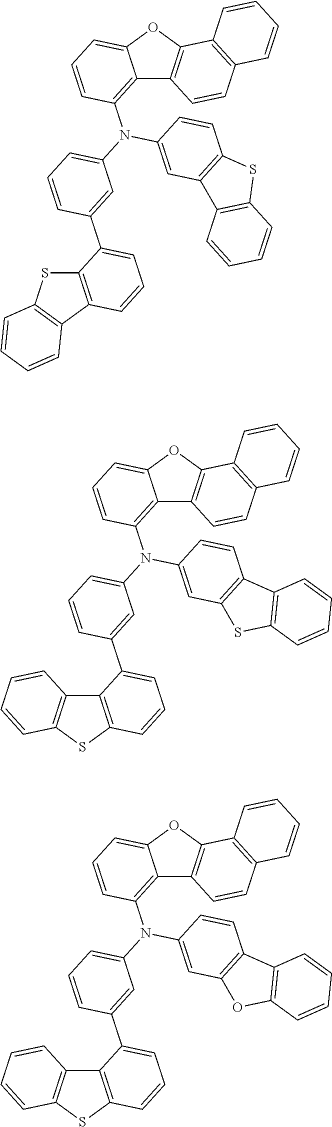 [SMILES: c1cc(-c2cccc3c2sc2ccccc23)cc(N(c2ccc3sc4ccccc4c3c2)c2cccc3oc4c5ccccc5ccc4c23)c1 ; c1cc(-c2cccc3sc4ccccc4c23)cc(N(c2ccc3c(c2)sc2ccccc23)c2cccc3oc4c5ccccc5ccc4c23)c1 ; c1cc(-c2cccc3sc4ccccc4c23)cc(N(c2ccc3c(c2)oc2ccccc23)c2cccc3oc4c5ccccc5ccc4c23)c1]

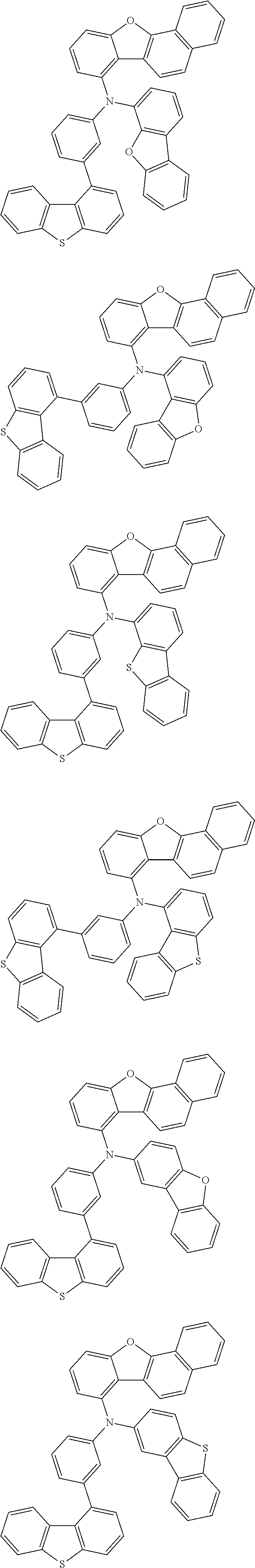 [SMILES: c1cc(-c2cccc3sc4ccccc4c23)cc(N(c2cccc3c2oc2ccccc23)c2cccc3oc4c5ccccc5ccc4c23)c1 ; c1cc(-c2cccc3sc4ccccc4c23)cc(N(c2cccc3oc4ccccc4c23)c2cccc3oc4c5ccccc5ccc4c23)c1 ; c1cc(-c2cccc3sc4ccccc4c23)cc(N(c2cccc3c2sc2ccccc23)c2cccc3oc4c5ccccc5ccc4c23)c1 ; c1cc(-c2cccc3sc4ccccc4c23)cc(N(c2cccc3oc4c5ccccc5ccc4c23)c2cccc3sc4ccccc4c23)c1 ; c1cc(-c2cccc3sc4ccccc4c23)cc(N(c2ccc3oc4ccccc4c3c2)c2cccc3oc4c5ccccc5ccc4c23)c1 ; c1cc(-c2cccc3sc4ccccc4c23)cc(N(c2ccc3sc4ccccc4c3c2)c2cccc3oc4c5ccccc5ccc4c23)c1]

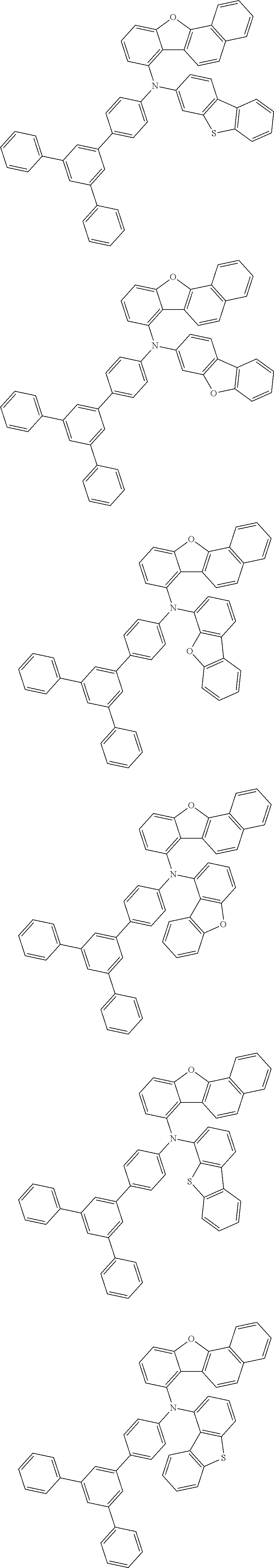

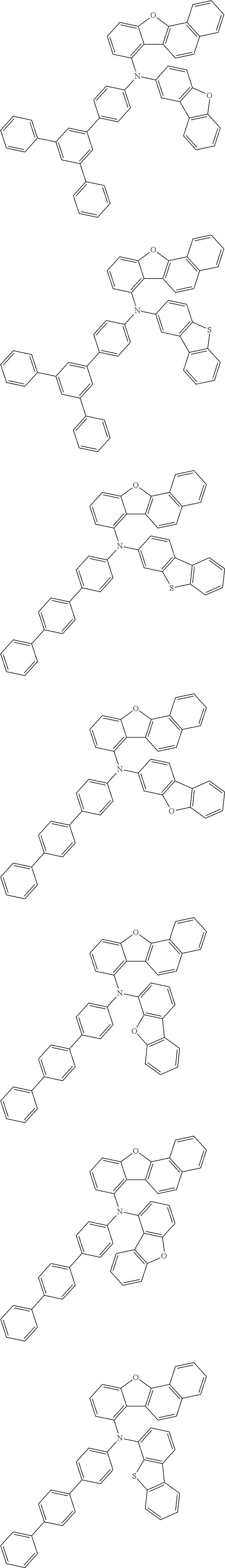

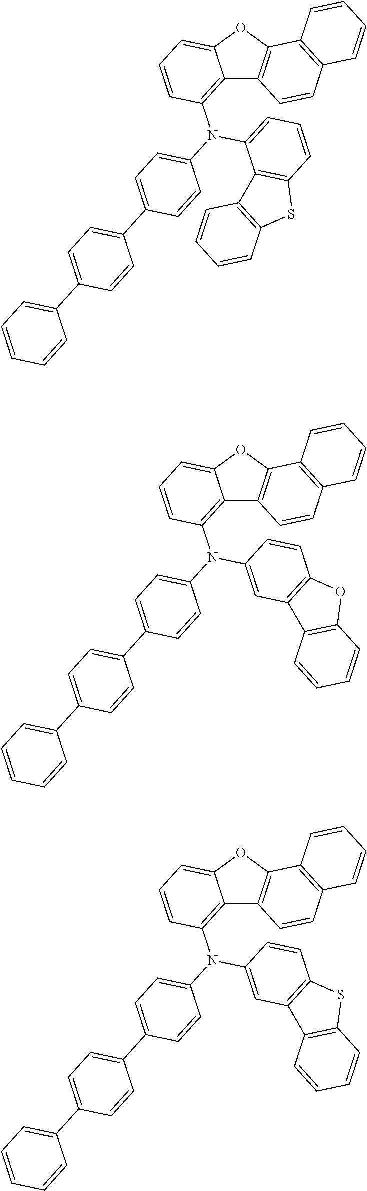 [SMILES: c1ccc(-c2ccc(-c3ccc(N(c4cccc5oc6c7ccccc7ccc6c45)c4cccc5sc6ccccc6c45)cc3)cc2)cc1 ; c1ccc(-c2ccc(-c3ccc(N(c4ccc5oc6ccccc6c5c4)c4cccc5oc6c7ccccc7ccc6c45)cc3)cc2)cc1 ; c1ccc(-c2ccc(-c3ccc(N(c4ccc5sc6ccccc6c5c4)c4cccc5oc6c7ccccc7ccc6c45)cc3)cc2)cc1]

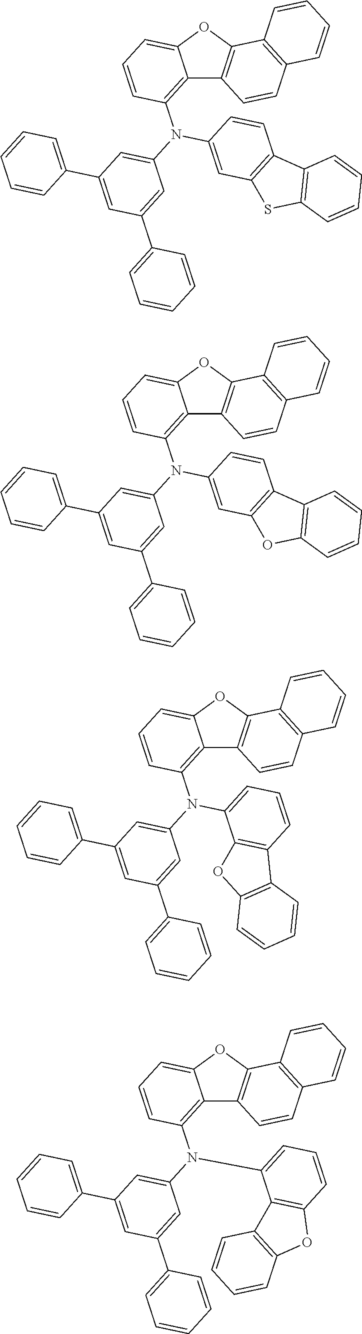 [SMILES: c1ccc(-c2cc(-c3ccccc3)cc(N(c3ccc4c(c3)sc3ccccc34)c3cccc4oc5c6ccccc6ccc5c34)c2)cc1 ; c1ccc(-c2cc(-c3ccccc3)cc(N(c3ccc4c(c3)oc3ccccc34)c3cccc4oc5c6ccccc6ccc5c34)c2)cc1 ; c1ccc(-c2cc(-c3ccccc3)cc(N(c3cccc4c3oc3ccccc34)c3cccc4oc5c6ccccc6ccc5c34)c2)cc1 ; c1ccc(-c2cc(-c3ccccc3)cc(N(c3cccc4oc5ccccc5c34)c3cccc4oc5c6ccccc6ccc5c34)c2)cc1]

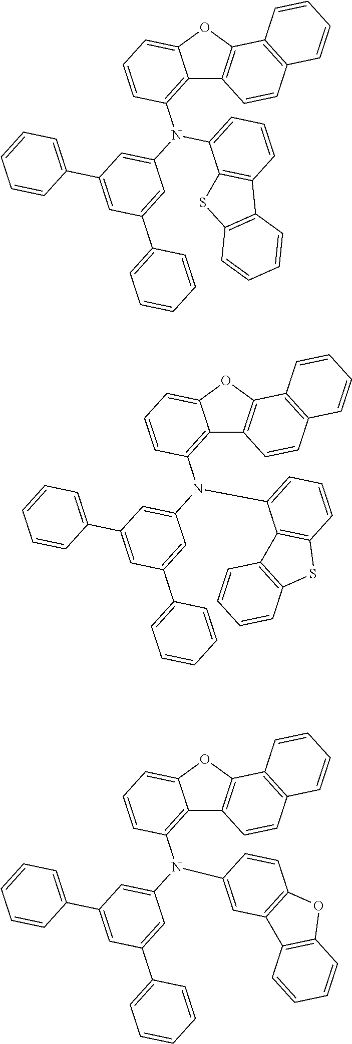 [SMILES: c1ccc(-c2cc(-c3ccccc3)cc(N(c3cccc4c3sc3ccccc34)c3cccc4oc5c6ccccc6ccc5c34)c2)cc1 ; c1ccc(-c2cc(-c3ccccc3)cc(N(c3cccc4oc5c6ccccc6ccc5c34)c3cccc4sc5ccccc5c34)c2)cc1 ; c1ccc(-c2cc(-c3ccccc3)cc(N(c3ccc4oc5ccccc5c4c3)c3cccc4oc5c6ccccc6ccc5c34)c2)cc1]

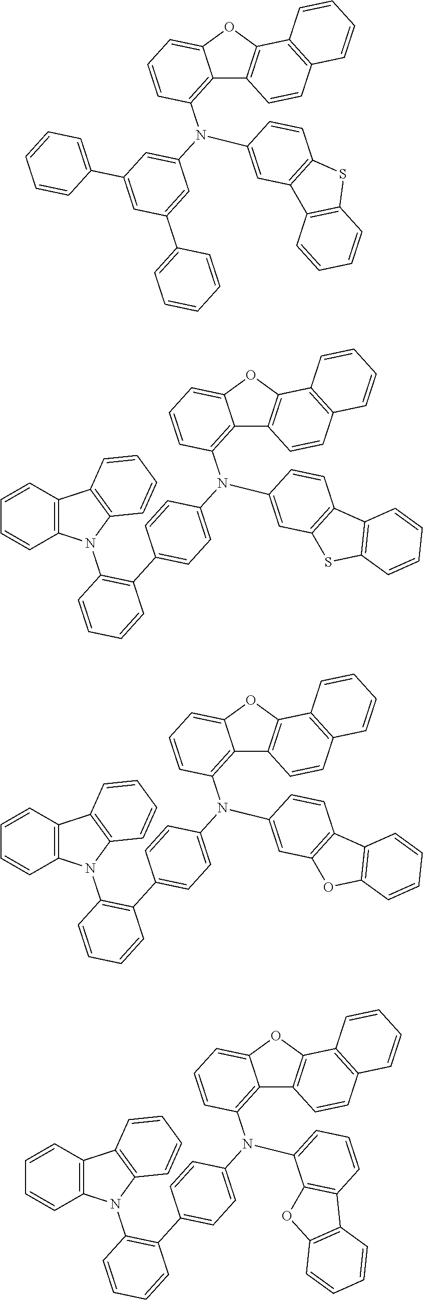 [SMILES: c1ccc(-c2cc(-c3ccccc3)cc(N(c3ccc4sc5ccccc5c4c3)c3cccc4oc5c6ccccc6ccc5c34)c2)cc1 ; c1ccc(-n2c3ccccc3c3ccccc32)c(-c2ccc(N(c3ccc4c(c3)sc3ccccc34)c3cccc4oc5c6ccccc6ccc5c34)cc2)c1 ; c1ccc(-n2c3ccccc3c3ccccc32)c(-c2ccc(N(c3ccc4c(c3)oc3ccccc34)c3cccc4oc5c6ccccc6ccc5c34)cc2)c1 ; c1ccc(-n2c3ccccc3c3ccccc32)c(-c2ccc(N(c3cccc4c3oc3ccccc34)c3cccc4oc5c6ccccc6ccc5c34)cc2)c1]

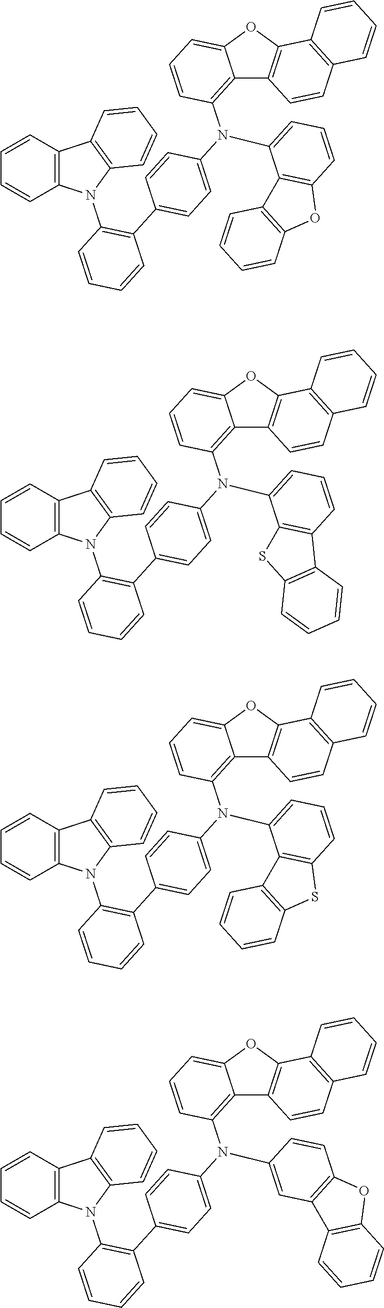 [SMILES: c1ccc(-n2c3ccccc3c3ccccc32)c(-c2ccc(N(c3cccc4oc5ccccc5c34)c3cccc4oc5c6ccccc6ccc5c34)cc2)c1 ; c1ccc(-n2c3ccccc3c3ccccc32)c(-c2ccc(N(c3cccc4c3sc3ccccc34)c3cccc4oc5c6ccccc6ccc5c34)cc2)c1 ; c1ccc(-n2c3ccccc3c3ccccc32)c(-c2ccc(N(c3cccc4oc5c6ccccc6ccc5c34)c3cccc4sc5ccccc5c34)cc2)c1 ; c1ccc(-n2c3ccccc3c3ccccc32)c(-c2ccc(N(c3ccc4oc5ccccc5c4c3)c3cccc4oc5c6ccccc6ccc5c34)cc2)c1]

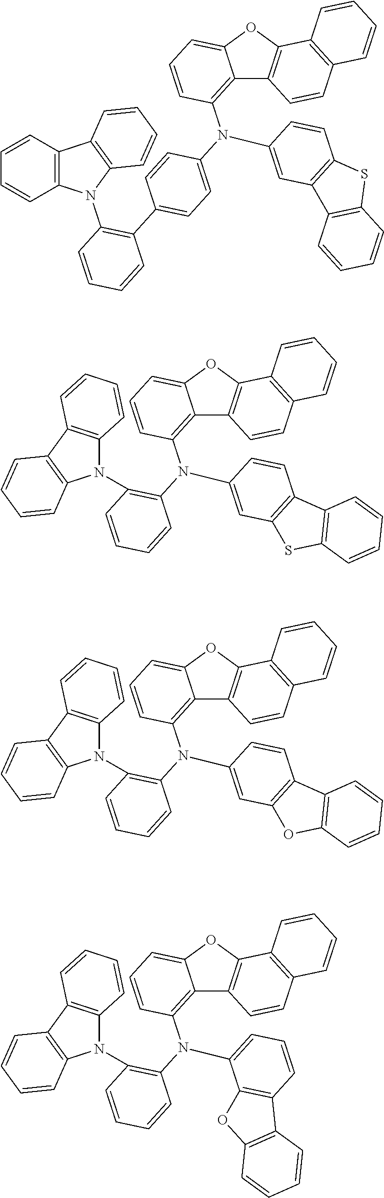 [SMILES: c1ccc(-n2c3ccccc3c3ccccc32)c(-c2ccc(N(c3ccc4sc5ccccc5c4c3)c3cccc4oc5c6ccccc6ccc5c34)cc2)c1 ; c1ccc(-n2c3ccccc3c3ccccc32)c(N(c2ccc3c(c2)sc2ccccc23)c2cccc3oc4c5ccccc5ccc4c23)c1 ; c1ccc(-n2c3ccccc3c3ccccc32)c(N(c2ccc3c(c2)oc2ccccc23)c2cccc3oc4c5ccccc5ccc4c23)c1 ; c1ccc(-n2c3ccccc3c3ccccc32)c(N(c2cccc3c2oc2ccccc23)c2cccc3oc4c5ccccc5ccc4c23)c1]

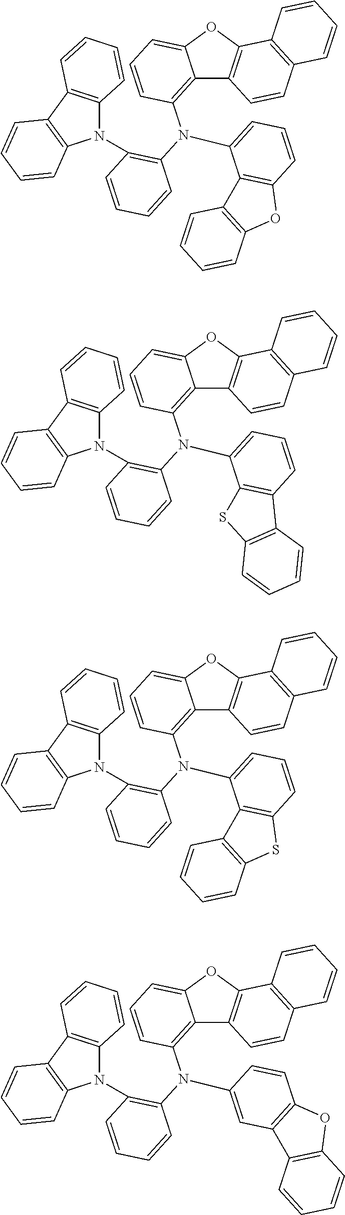 [SMILES: c1ccc(-n2c3ccccc3c3ccccc32)c(N(c2cccc3oc4ccccc4c23)c2cccc3oc4c5ccccc5ccc4c23)c1 ; c1ccc(-n2c3ccccc3c3ccccc32)c(N(c2cccc3c2sc2ccccc23)c2cccc3oc4c5ccccc5ccc4c23)c1 ; c1ccc(-n2c3ccccc3c3ccccc32)c(N(c2cccc3oc4c5ccccc5ccc4c23)c2cccc3sc4ccccc4c23)c1 ; c1ccc(-n2c3ccccc3c3ccccc32)c(N(c2ccc3oc4ccccc4c3c2)c2cccc3oc4c5ccccc5ccc4c23)c1]

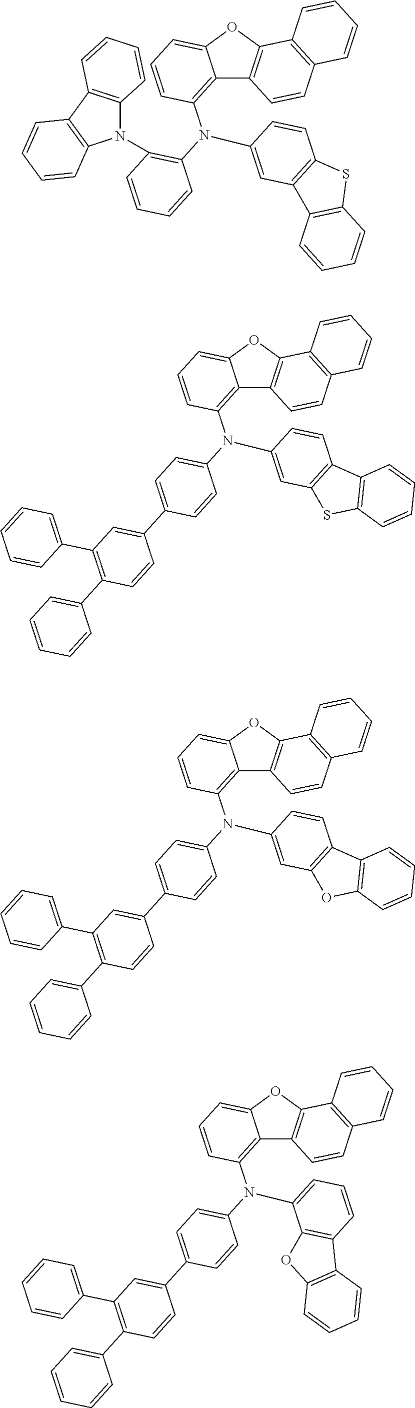 [SMILES: c1ccc(-n2c3ccccc3c3ccccc32)c(N(c2ccc3sc4ccccc4c3c2)c2cccc3oc4c5ccccc5ccc4c23)c1 ; c1ccc(-c2ccc(-c3ccc(N(c4ccc5c(c4)sc4ccccc45)c4cccc5oc6c7ccccc7ccc6c45)cc3)cc2-c2ccccc2)cc1 ; c1ccc(-c2ccc(-c3ccc(N(c4ccc5c(c4)oc4ccccc45)c4cccc5oc6c7ccccc7ccc6c45)cc3)cc2-c2ccccc2)cc1 ; c1ccc(-c2ccc(-c3ccc(N(c4cccc5c4oc4ccccc45)c4cccc5oc6c7ccccc7ccc6c45)cc3)cc2-c2ccccc2)cc1]

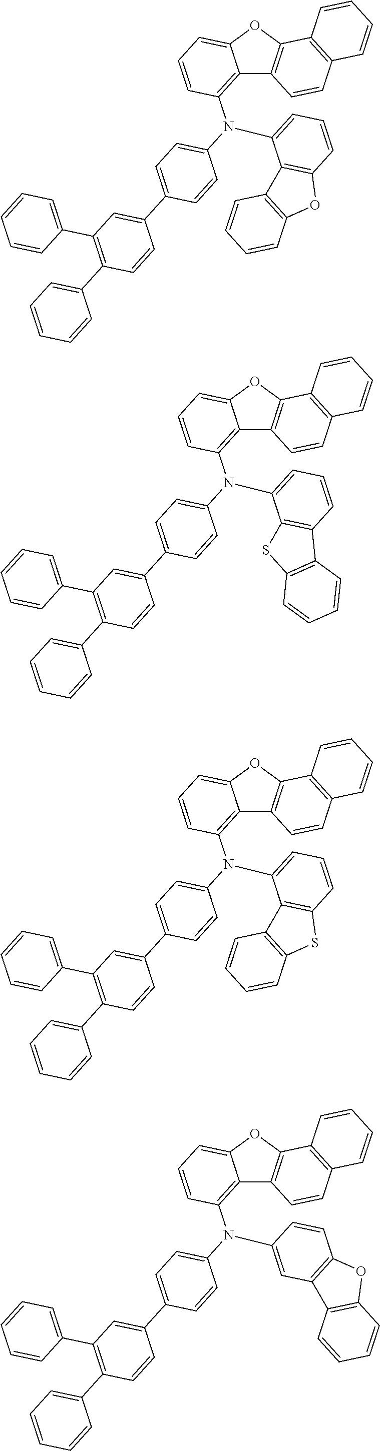 [SMILES: c1ccc(-c2ccc(-c3ccc(N(c4cccc5oc6ccccc6c45)c4cccc5oc6c7ccccc7ccc6c45)cc3)cc2-c2ccccc2)cc1 ; c1ccc(-c2ccc(-c3ccc(N(c4cccc5c4sc4ccccc45)c4cccc5oc6c7ccccc7ccc6c45)cc3)cc2-c2ccccc2)cc1 ; c1ccc(-c2ccc(-c3ccc(N(c4cccc5oc6c7ccccc7ccc6c45)c4cccc5sc6ccccc6c45)cc3)cc2-c2ccccc2)cc1 ; c1ccc(-c2ccc(-c3ccc(N(c4ccc5oc6ccccc6c5c4)c4cccc5oc6c7ccccc7ccc6c45)cc3)cc2-c2ccccc2)cc1]

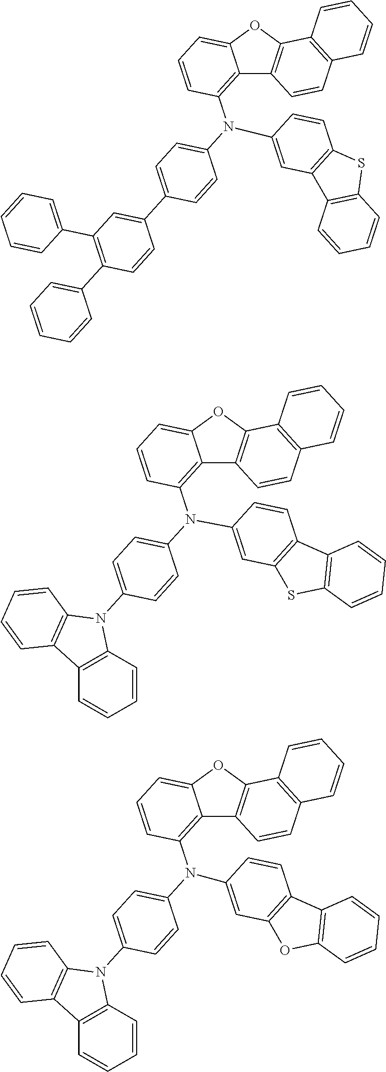 [SMILES: c1ccc(-c2ccc(-c3ccc(N(c4ccc5sc6ccccc6c5c4)c4cccc5oc6c7ccccc7ccc6c45)cc3)cc2-c2ccccc2)cc1 ; c1ccc2c(c1)ccc1c2oc2cccc(N(c3ccc(-n4c5ccccc5c5ccccc54)cc3)c3ccc4c(c3)sc3ccccc34)c21 ; c1ccc2c(c1)ccc1c2oc2cccc(N(c3ccc(-n4c5ccccc5c5ccccc54)cc3)c3ccc4c(c3)oc3ccccc34)c21]

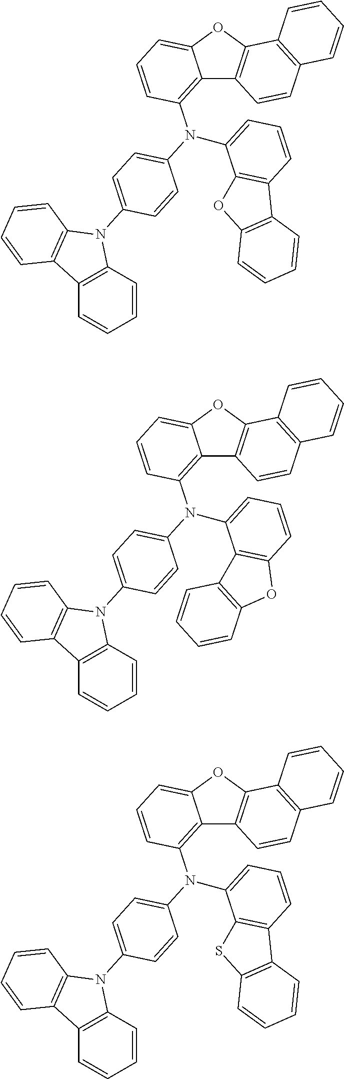 [SMILES: c1ccc2c(c1)ccc1c2oc2cccc(N(c3ccc(-n4c5ccccc5c5ccccc54)cc3)c3cccc4c3oc3ccccc34)c21 ; c1ccc2c(c1)ccc1c2oc2cccc(N(c3ccc(-n4c5ccccc5c5ccccc54)cc3)c3cccc4oc5ccccc5c34)c21 ; c1ccc2c(c1)ccc1c2oc2cccc(N(c3ccc(-n4c5ccccc5c5ccccc54)cc3)c3cccc4c3sc3ccccc34)c21]

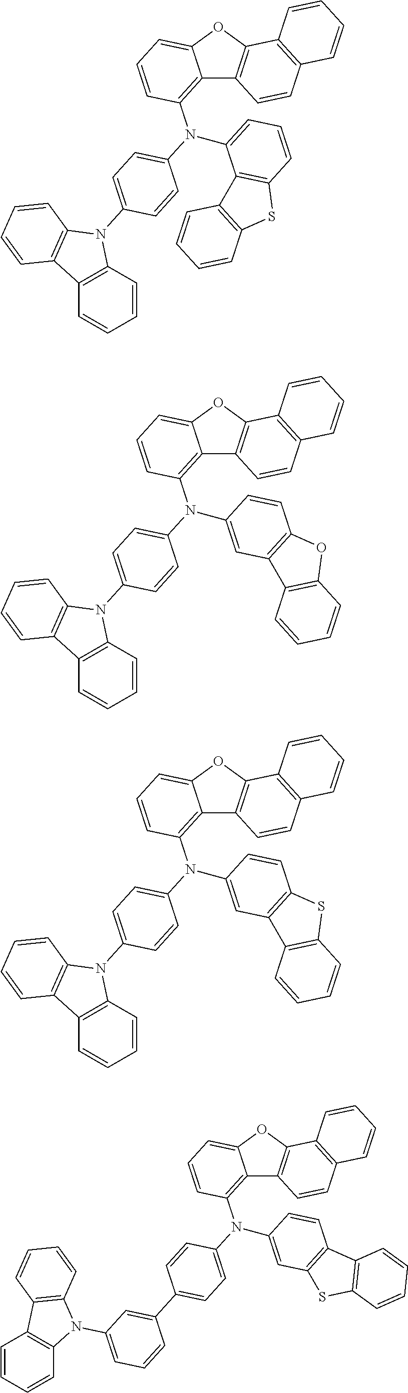 [SMILES: c1ccc2c(c1)ccc1c2oc2cccc(N(c3ccc(-n4c5ccccc5c5ccccc54)cc3)c3cccc4sc5ccccc5c34)c21 ; c1ccc2c(c1)ccc1c2oc2cccc(N(c3ccc(-n4c5ccccc5c5ccccc54)cc3)c3ccc4oc5ccccc5c4c3)c21 ; c1ccc2c(c1)ccc1c2oc2cccc(N(c3ccc(-n4c5ccccc5c5ccccc54)cc3)c3ccc4sc5ccccc5c4c3)c21 ; c1cc(-c2ccc(N(c3ccc4c(c3)sc3ccccc34)c3cccc4oc5c6ccccc6ccc5c34)cc2)cc(-n2c3ccccc3c3ccccc32)c1]

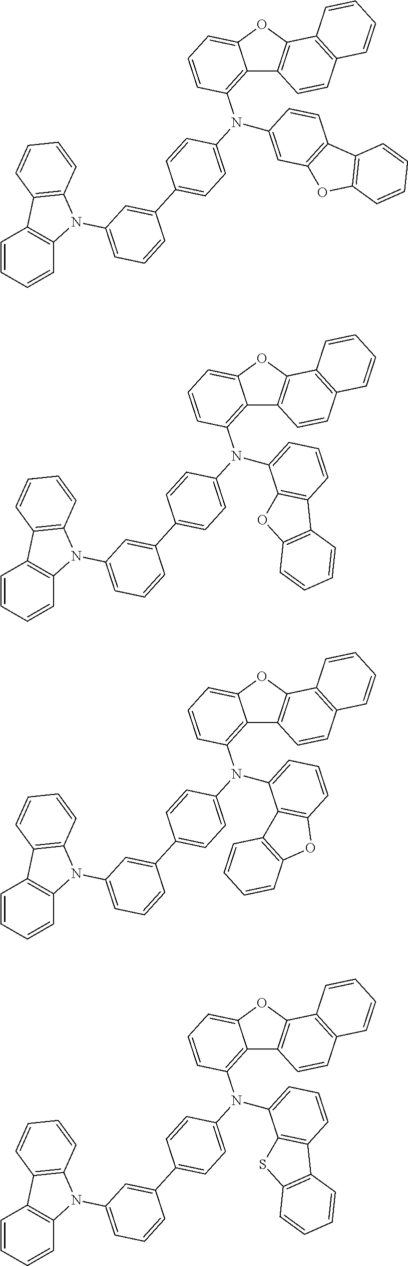 [SMILES: c1cc(-c2ccc(N(c3ccc4c(c3)oc3ccccc34)c3cccc4oc5c6ccccc6ccc5c34)cc2)cc(-n2c3ccccc3c3ccccc32)c1 ; c1cc(-c2ccc(N(c3cccc4c3oc3ccccc34)c3cccc4oc5c6ccccc6ccc5c34)cc2)cc(-n2c3ccccc3c3ccccc32)c1 ; c1cc(-c2ccc(N(c3cccc4oc5ccccc5c34)c3cccc4oc5c6ccccc6ccc5c34)cc2)cc(-n2c3ccccc3c3ccccc32)c1 ; c1cc(-c2ccc(N(c3cccc4c3sc3ccccc34)c3cccc4oc5c6ccccc6ccc5c34)cc2)cc(-n2c3ccccc3c3ccccc32)c1]

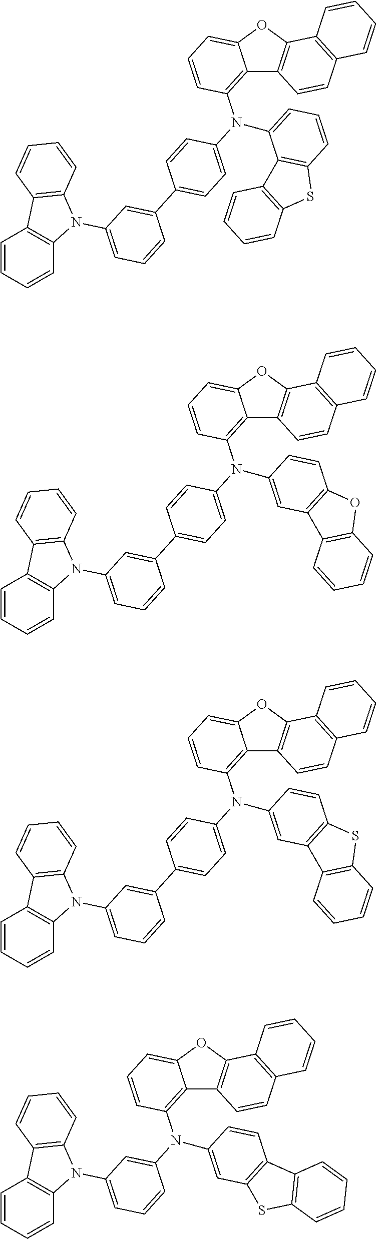 [SMILES: c1cc(-c2ccc(N(c3cccc4oc5c6ccccc6ccc5c34)c3cccc4sc5ccccc5c34)cc2)cc(-n2c3ccccc3c3ccccc32)c1 ; c1cc(-c2ccc(N(c3ccc4oc5ccccc5c4c3)c3cccc4oc5c6ccccc6ccc5c34)cc2)cc(-n2c3ccccc3c3ccccc32)c1 ; c1cc(-c2ccc(N(c3ccc4sc5ccccc5c4c3)c3cccc4oc5c6ccccc6ccc5c34)cc2)cc(-n2c3ccccc3c3ccccc32)c1 ; c1cc(N(c2ccc3c(c2)sc2ccccc23)c2cccc3oc4c5ccccc5ccc4c23)cc(-n2c3ccccc3c3ccccc32)c1]

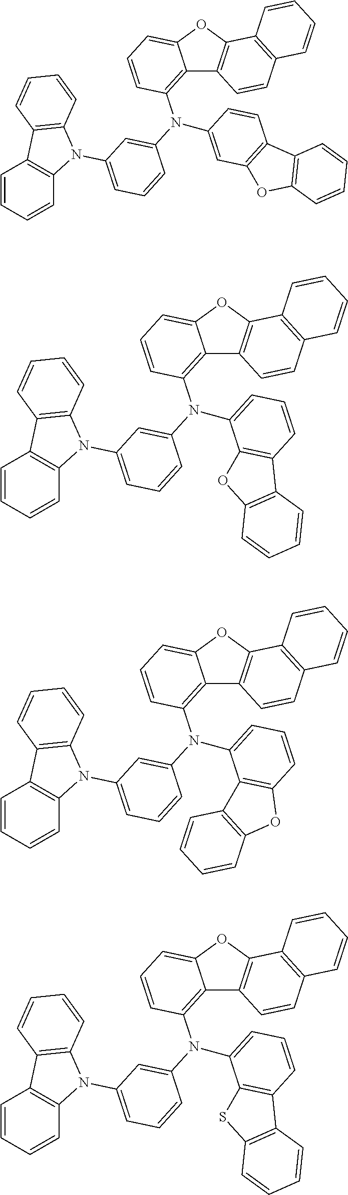 [SMILES: c1cc(N(c2ccc3c(c2)oc2ccccc23)c2cccc3oc4c5ccccc5ccc4c23)cc(-n2c3ccccc3c3ccccc32)c1 ; c1cc(N(c2cccc3c2oc2ccccc23)c2cccc3oc4c5ccccc5ccc4c23)cc(-n2c3ccccc3c3ccccc32)c1 ; c1cc(N(c2cccc3oc4ccccc4c23)c2cccc3oc4c5ccccc5ccc4c23)cc(-n2c3ccccc3c3ccccc32)c1 ; c1cc(N(c2cccc3c2sc2ccccc23)c2cccc3oc4c5ccccc5ccc4c23)cc(-n2c3ccccc3c3ccccc32)c1]

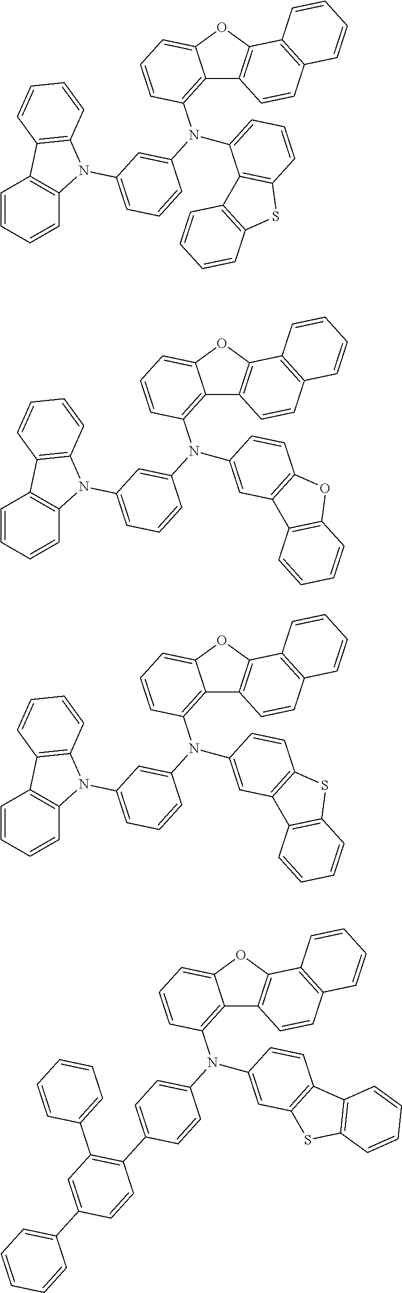

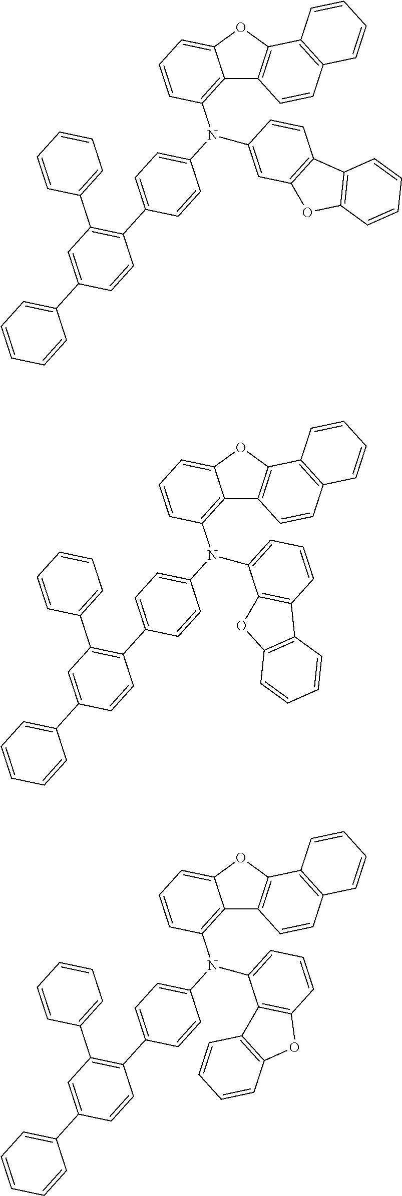 [SMILES: c1ccc(-c2ccc(-c3ccc(N(c4ccc5c(c4)oc4ccccc45)c4cccc5oc6c7ccccc7ccc6c45)cc3)c(-c3ccccc3)c2)cc1 ; c1ccc(-c2ccc(-c3ccc(N(c4cccc5c4oc4ccccc45)c4cccc5oc6c7ccccc7ccc6c45)cc3)c(-c3ccccc3)c2)cc1 ; c1ccc(-c2ccc(-c3ccc(N(c4cccc5oc6ccccc6c45)c4cccc5oc6c7ccccc7ccc6c45)cc3)c(-c3ccccc3)c2)cc1]

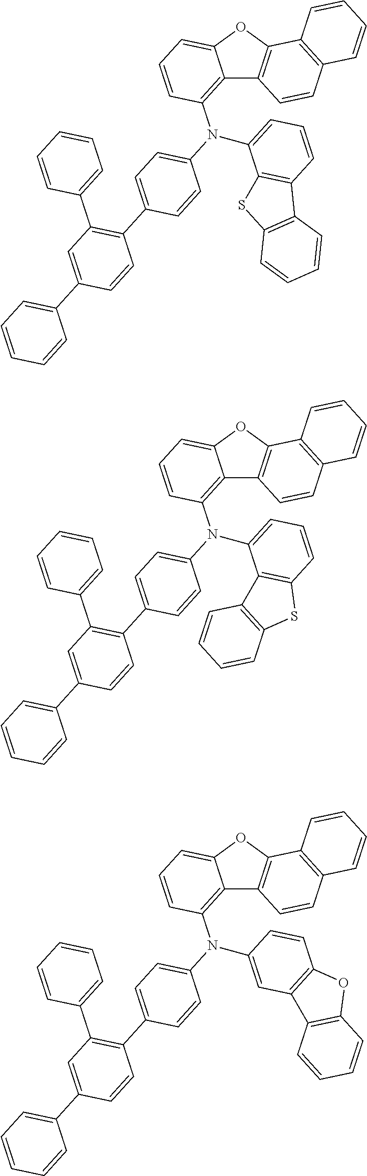 [SMILES: c1ccc(-c2ccc(-c3ccc(N(c4cccc5c4sc4ccccc45)c4cccc5oc6c7ccccc7ccc6c45)cc3)c(-c3ccccc3)c2)cc1 ; c1ccc(-c2ccc(-c3ccc(N(c4cccc5oc6c7ccccc7ccc6c45)c4cccc5sc6ccccc6c45)cc3)c(-c3ccccc3)c2)cc1 ; c1ccc(-c2ccc(-c3ccc(N(c4ccc5oc6ccccc6c5c4)c4cccc5oc6c7ccccc7ccc6c45)cc3)c(-c3ccccc3)c2)cc1]

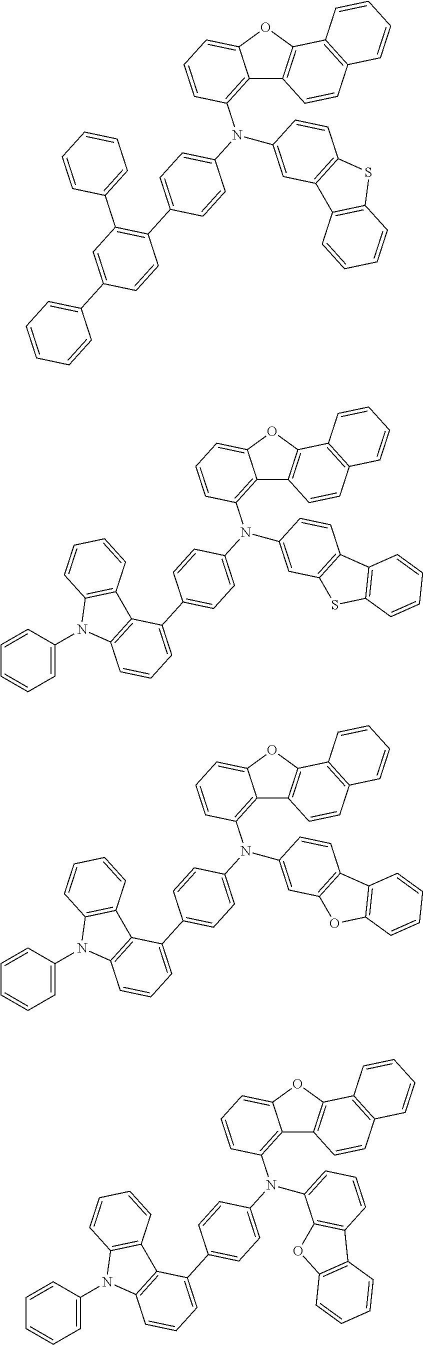 [SMILES: c1ccc(-c2ccc(-c3ccc(N(c4ccc5sc6ccccc6c5c4)c4cccc5oc6c7ccccc7ccc6c45)cc3)c(-c3ccccc3)c2)cc1 ; c1ccc(-n2c3ccccc3c3c(-c4ccc(N(c5ccc6c(c5)sc5ccccc56)c5cccc6oc7c8ccccc8ccc7c56)cc4)cccc32)cc1 ; c1ccc(-n2c3ccccc3c3c(-c4ccc(N(c5ccc6c(c5)oc5ccccc56)c5cccc6oc7c8ccccc8ccc7c56)cc4)cccc32)cc1 ; c1ccc(-n2c3ccccc3c3c(-c4ccc(N(c5cccc6c5oc5ccccc56)c5cccc6oc7c8ccccc8ccc7c56)cc4)cccc32)cc1]

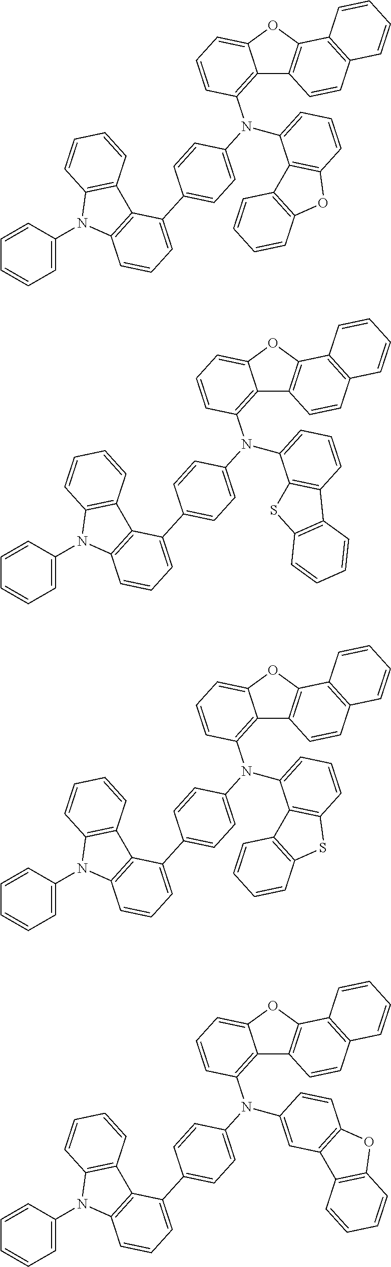 [SMILES: c1ccc(-n2c3ccccc3c3c(-c4ccc(N(c5cccc6oc7ccccc7c56)c5cccc6oc7c8ccccc8ccc7c56)cc4)cccc32)cc1 ; c1ccc(-n2c3ccccc3c3c(-c4ccc(N(c5cccc6c5sc5ccccc56)c5cccc6oc7c8ccccc8ccc7c56)cc4)cccc32)cc1 ; c1ccc(-n2c3ccccc3c3c(-c4ccc(N(c5cccc6oc7c8ccccc8ccc7c56)c5cccc6sc7ccccc7c56)cc4)cccc32)cc1 ; c1ccc(-n2c3ccccc3c3c(-c4ccc(N(c5ccc6oc7ccccc7c6c5)c5cccc6oc7c8ccccc8ccc7c56)cc4)cccc32)cc1]

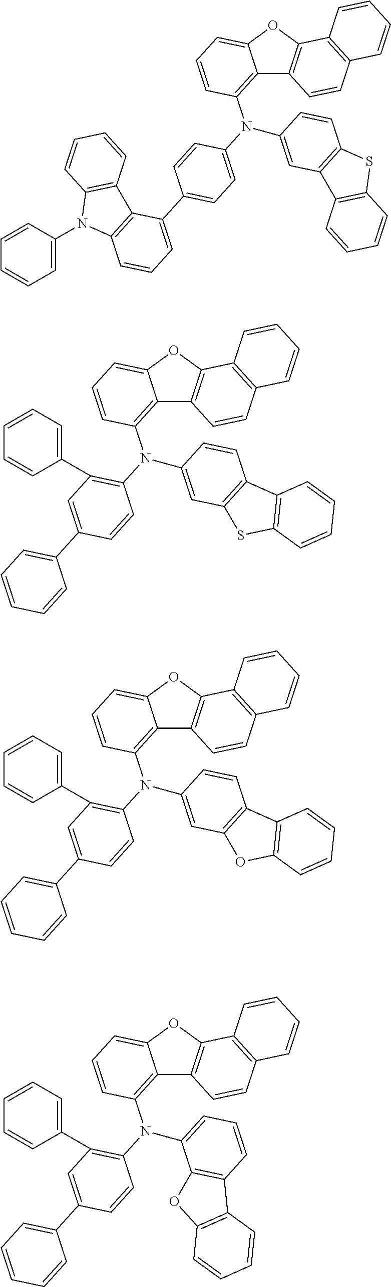 [SMILES: c1ccc(-n2c3ccccc3c3c(-c4ccc(N(c5ccc6sc7ccccc7c6c5)c5cccc6oc7c8ccccc8ccc7c56)cc4)cccc32)cc1 ; c1ccc(-c2ccc(N(c3ccc4c(c3)sc3ccccc34)c3cccc4oc5c6ccccc6ccc5c34)c(-c3ccccc3)c2)cc1 ; c1ccc(-c2ccc(N(c3ccc4c(c3)oc3ccccc34)c3cccc4oc5c6ccccc6ccc5c34)c(-c3ccccc3)c2)cc1 ; c1ccc(-c2ccc(N(c3cccc4c3oc3ccccc34)c3cccc4oc5c6ccccc6ccc5c34)c(-c3ccccc3)c2)cc1]

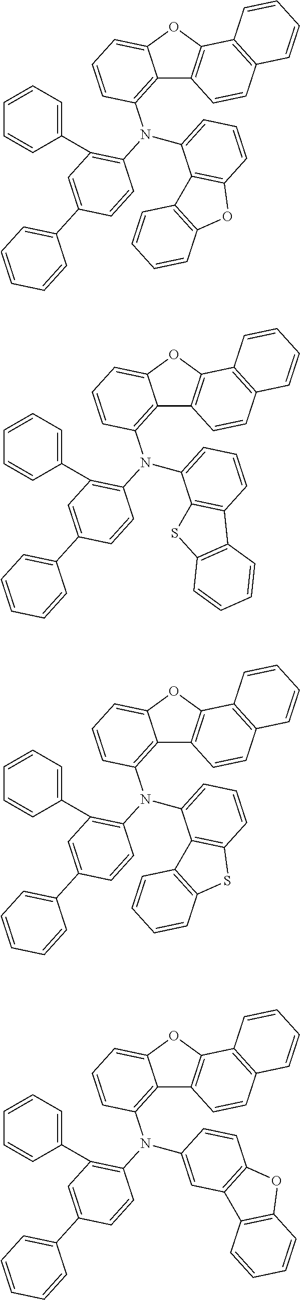 [SMILES: c1ccc(-c2ccc(N(c3cccc4oc5ccccc5c34)c3cccc4oc5c6ccccc6ccc5c34)c(-c3ccccc3)c2)cc1 ; c1ccc(-c2ccc(N(c3cccc4c3sc3ccccc34)c3cccc4oc5c6ccccc6ccc5c34)c(-c3ccccc3)c2)cc1 ; c1ccc(-c2ccc(N(c3cccc4oc5c6ccccc6ccc5c34)c3cccc4sc5ccccc5c34)c(-c3ccccc3)c2)cc1 ; c1ccc(-c2ccc(N(c3ccc4oc5ccccc5c4c3)c3cccc4oc5c6ccccc6ccc5c34)c(-c3ccccc3)c2)cc1]

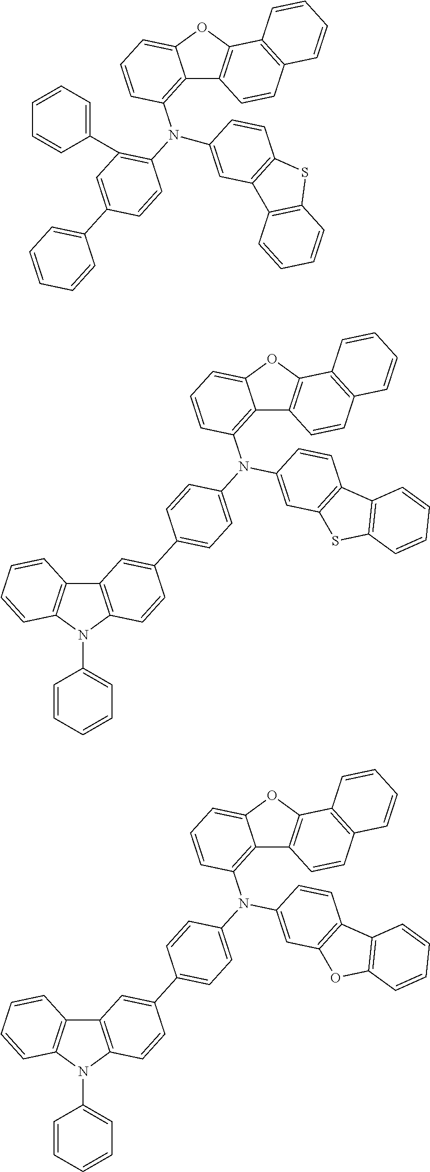 [SMILES: c1ccc(-c2ccc(N(c3ccc4sc5ccccc5c4c3)c3cccc4oc5c6ccccc6ccc5c34)c(-c3ccccc3)c2)cc1 ; c1ccc(-n2c3ccccc3c3cc(-c4ccc(N(c5ccc6c(c5)sc5ccccc56)c5cccc6oc7c8ccccc8ccc7c56)cc4)ccc32)cc1 ; c1ccc(-n2c3ccccc3c3cc(-c4ccc(N(c5ccc6c(c5)oc5ccccc56)c5cccc6oc7c8ccccc8ccc7c56)cc4)ccc32)cc1]

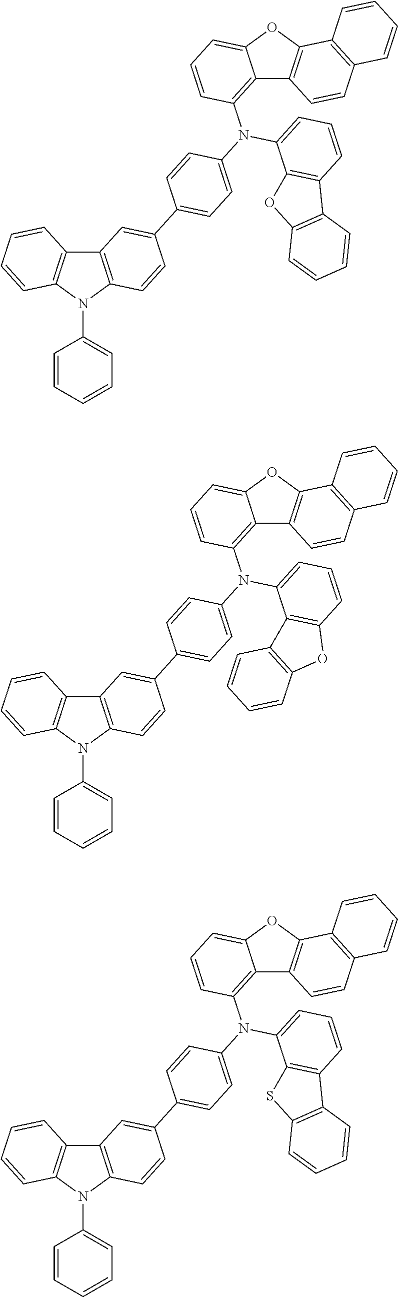 [SMILES: c1ccc(-n2c3ccccc3c3cc(-c4ccc(N(c5cccc6c5oc5ccccc56)c5cccc6oc7c8ccccc8ccc7c56)cc4)ccc32)cc1 ; c1ccc(-n2c3ccccc3c3cc(-c4ccc(N(c5cccc6oc7ccccc7c56)c5cccc6oc7c8ccccc8ccc7c56)cc4)ccc32)cc1 ; c1ccc(-n2c3ccccc3c3cc(-c4ccc(N(c5cccc6c5sc5ccccc56)c5cccc6oc7c8ccccc8ccc7c56)cc4)ccc32)cc1]

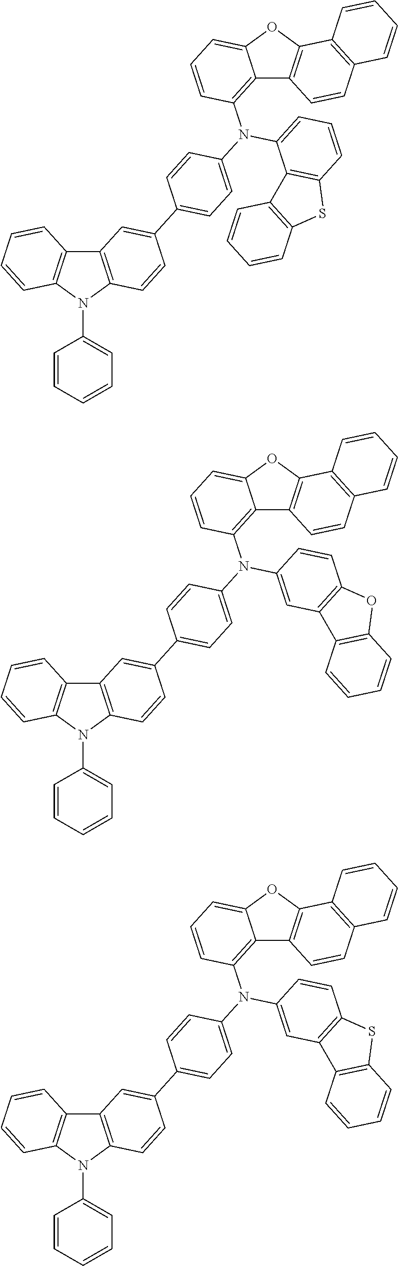 [SMILES: c1ccc(-n2c3ccccc3c3cc(-c4ccc(N(c5cccc6oc7c8ccccc8ccc7c56)c5cccc6sc7ccccc7c56)cc4)ccc32)cc1 ; c1ccc(-n2c3ccccc3c3cc(-c4ccc(N(c5ccc6oc7ccccc7c6c5)c5cccc6oc7c8ccccc8ccc7c56)cc4)ccc32)cc1 ; c1ccc(-n2c3ccccc3c3cc(-c4ccc(N(c5ccc6sc7ccccc7c6c5)c5cccc6oc7c8ccccc8ccc7c56)cc4)ccc32)cc1]

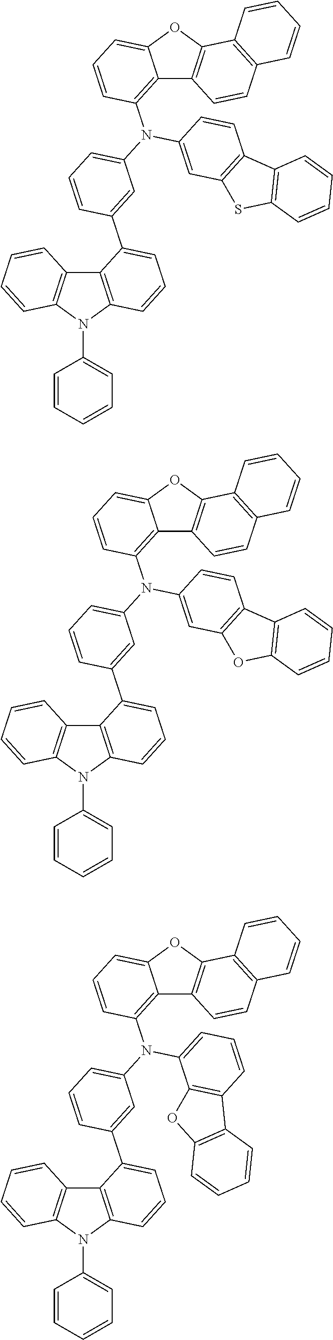 [SMILES: c1ccc(-n2c3ccccc3c3c(-c4cccc(N(c5ccc6c(c5)sc5ccccc56)c5cccc6oc7c8ccccc8ccc7c56)c4)cccc32)cc1 ; c1ccc(-n2c3ccccc3c3c(-c4cccc(N(c5ccc6c(c5)oc5ccccc56)c5cccc6oc7c8ccccc8ccc7c56)c4)cccc32)cc1 ; c1ccc(-n2c3ccccc3c3c(-c4cccc(N(c5cccc6c5oc5ccccc56)c5cccc6oc7c8ccccc8ccc7c56)c4)cccc32)cc1]

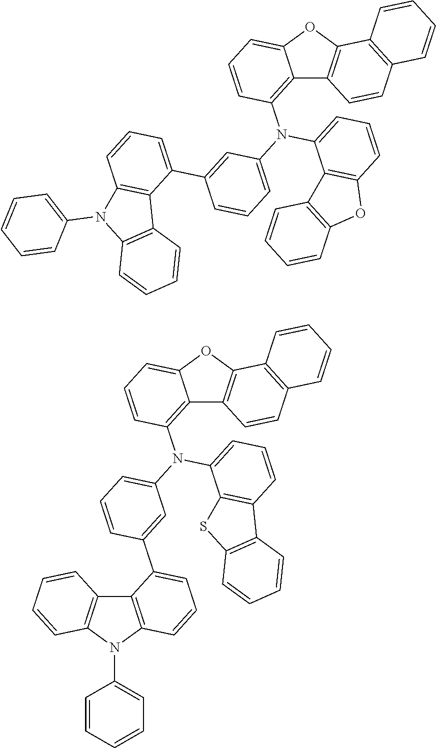 [SMILES: c1ccc(-n2c3ccccc3c3c(-c4cccc(N(c5cccc6oc7ccccc7c56)c5cccc6oc7c8ccccc8ccc7c56)c4)cccc32)cc1 ; c1ccc(-n2c3ccccc3c3c(-c4cccc(N(c5cccc6c5sc5ccccc56)c5cccc6oc7c8ccccc8ccc7c56)c4)cccc32)cc1]

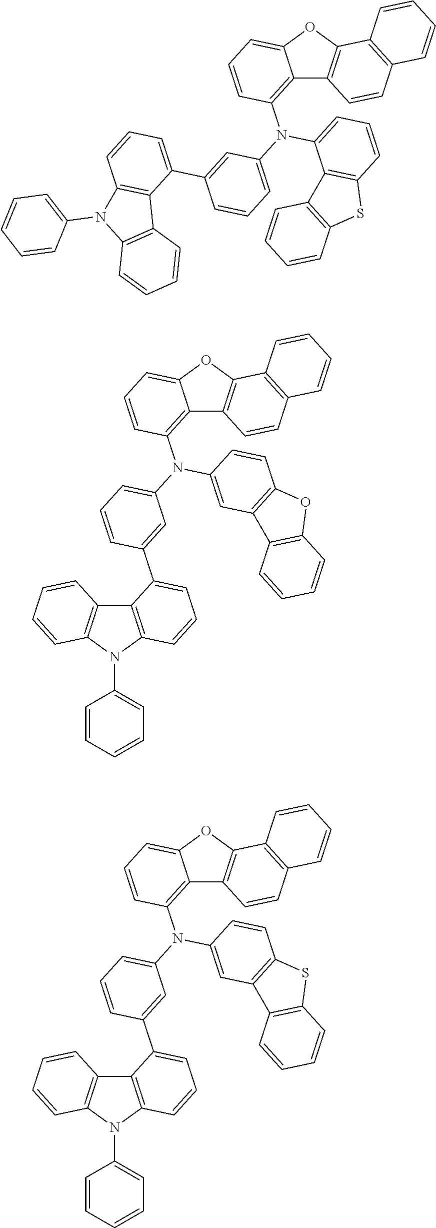 [SMILES: c1ccc(-n2c3ccccc3c3c(-c4cccc(N(c5cccc6oc7c8ccccc8ccc7c56)c5cccc6sc7ccccc7c56)c4)cccc32)cc1 ; c1ccc(-n2c3ccccc3c3c(-c4cccc(N(c5ccc6oc7ccccc7c6c5)c5cccc6oc7c8ccccc8ccc7c56)c4)cccc32)cc1 ; c1ccc(-n2c3ccccc3c3c(-c4cccc(N(c5ccc6sc7ccccc7c6c5)c5cccc6oc7c8ccccc8ccc7c56)c4)cccc32)cc1]

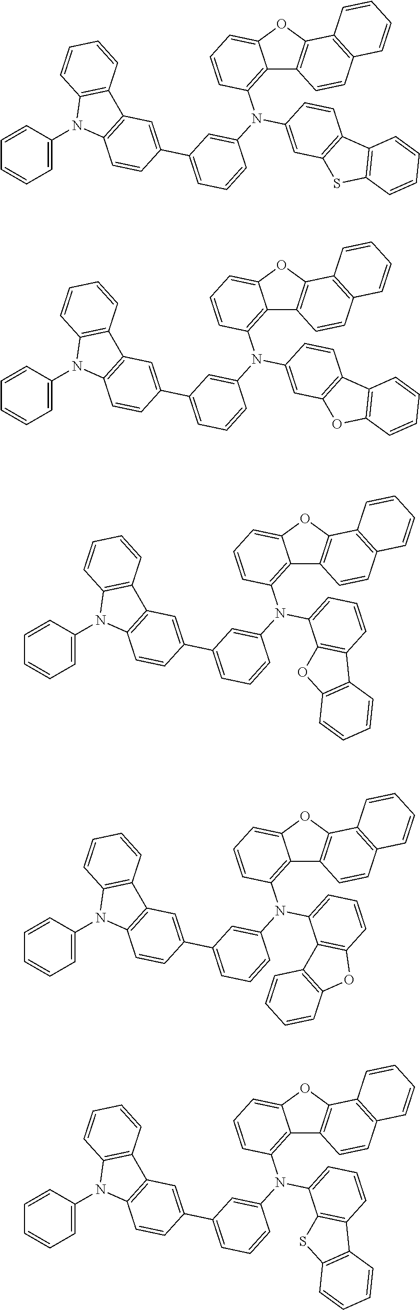 [SMILES: c1ccc(-n2c3ccccc3c3cc(-c4cccc(N(c5ccc6c(c5)sc5ccccc56)c5cccc6oc7c8ccccc8ccc7c56)c4)ccc32)cc1 ; c1ccc(-n2c3ccccc3c3cc(-c4cccc(N(c5ccc6c(c5)oc5ccccc56)c5cccc6oc7c8ccccc8ccc7c56)c4)ccc32)cc1 ; c1ccc(-n2c3ccccc3c3cc(-c4cccc(N(c5cccc6c5oc5ccccc56)c5cccc6oc7c8ccccc8ccc7c56)c4)ccc32)cc1 ; c1ccc(-n2c3ccccc3c3cc(-c4cccc(N(c5cccc6oc7ccccc7c56)c5cccc6oc7c8ccccc8ccc7c56)c4)ccc32)cc1 ; c1ccc(-n2c3ccccc3c3cc(-c4cccc(N(c5cccc6c5sc5ccccc56)c5cccc6oc7c8ccccc8ccc7c56)c4)ccc32)cc1]

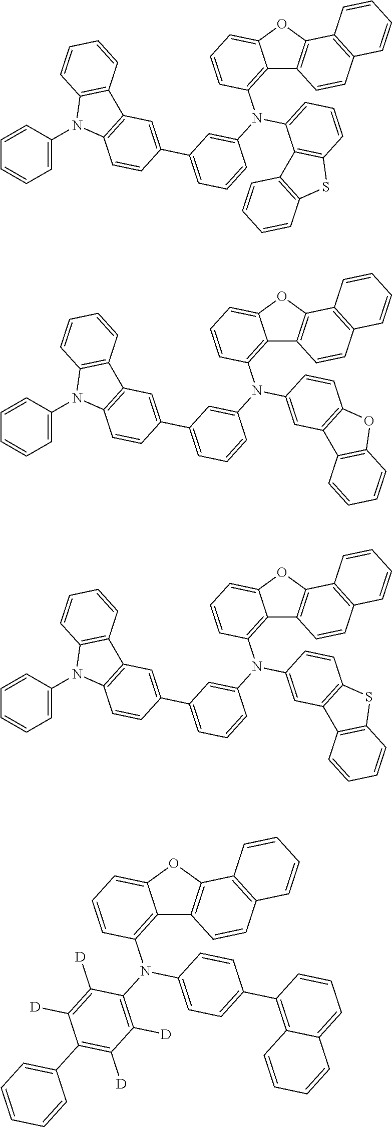 [SMILES: c1ccc(-n2c3ccccc3c3cc(-c4cccc(N(c5cccc6oc7c8ccccc8ccc7c56)c5cccc6sc7ccccc7c56)c4)ccc32)cc1 ; c1ccc(-n2c3ccccc3c3cc(-c4cccc(N(c5ccc6oc7ccccc7c6c5)c5cccc6oc7c8ccccc8ccc7c56)c4)ccc32)cc1 ; c1ccc(-n2c3ccccc3c3cc(-c4cccc(N(c5ccc6sc7ccccc7c6c5)c5cccc6oc7c8ccccc8ccc7c56)c4)ccc32)cc1 ; [2H]c1c([2H])c(N(c2ccc(-c3cccc4ccccc34)cc2)c2cccc3oc4c5ccccc5ccc4c23)c([2H])c([2H])c1-c1ccccc1]

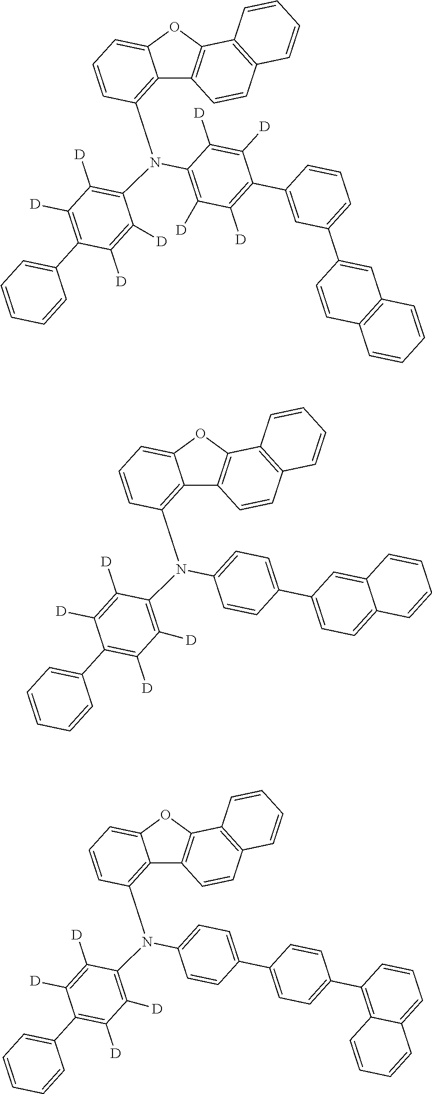 [SMILES: [2H]c1c([2H])c(N(c2c([2H])c([2H])c(-c3cccc(-c4ccc5ccccc5c4)c3)c([2H])c2[2H])c2cccc3oc4c5ccccc5ccc4c23)c([2H])c([2H])c1-c1ccccc1 ; [2H]c1c([2H])c(N(c2ccc(-c3ccc4ccccc4c3)cc2)c2cccc3oc4c5ccccc5ccc4c23)c([2H])c([2H])c1-c1ccccc1 ; [2H]c1c([2H])c(N(c2ccc(-c3ccc(-c4cccc5ccccc45)cc3)cc2)c2cccc3oc4c5ccccc5ccc4c23)c([2H])c([2H])c1-c1ccccc1]

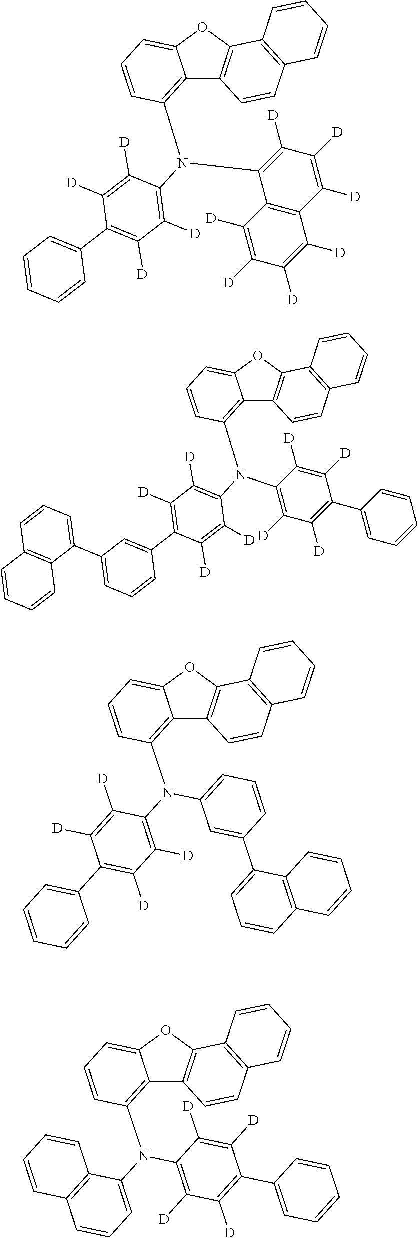 [SMILES: [2H]c1c([2H])c(N(c2c([2H])c([2H])c([2H])c3c([2H])c([2H])c([2H])c([2H])c23)c2cccc3oc4c5ccccc5ccc4c23)c([2H])c([2H])c1-c1ccccc1 ; [2H]c1c([2H])c(N(c2c([2H])c([2H])c(-c3cccc(-c4cccc5ccccc45)c3)c([2H])c2[2H])c2cccc3oc4c5ccccc5ccc4c23)c([2H])c([2H])c1-c1ccccc1 ; [2H]c1c([2H])c(N(c2cccc(-c3cccc4ccccc34)c2)c2cccc3oc4c5ccccc5ccc4c23)c([2H])c([2H])c1-c1ccccc1 ; [2H]c1c([2H])c(N(c2cccc3ccccc23)c2cccc3oc4c5ccccc5ccc4c23)c([2H])c([2H])c1-c1ccccc1]

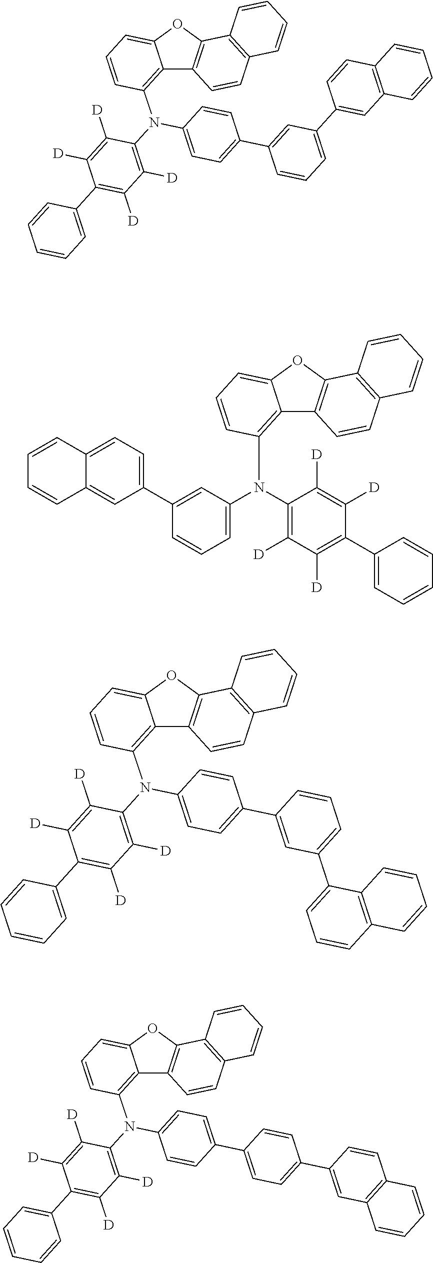 [SMILES: [2H]c1c([2H])c(N(c2ccc(-c3cccc(-c4ccc5ccccc5c4)c3)cc2)c2cccc3oc4c5ccccc5ccc4c23)c([2H])c([2H])c1-c1ccccc1 ; [2H]c1c([2H])c(N(c2cccc(-c3ccc4ccccc4c3)c2)c2cccc3oc4c5ccccc5ccc4c23)c([2H])c([2H])c1-c1ccccc1 ; [2H]c1c([2H])c(N(c2ccc(-c3cccc(-c4cccc5ccccc45)c3)cc2)c2cccc3oc4c5ccccc5ccc4c23)c([2H])c([2H])c1-c1ccccc1 ; [2H]c1c([2H])c(N(c2ccc(-c3ccc(-c4ccc5ccccc5c4)cc3)cc2)c2cccc3oc4c5ccccc5ccc4c23)c([2H])c([2H])c1-c1ccccc1]

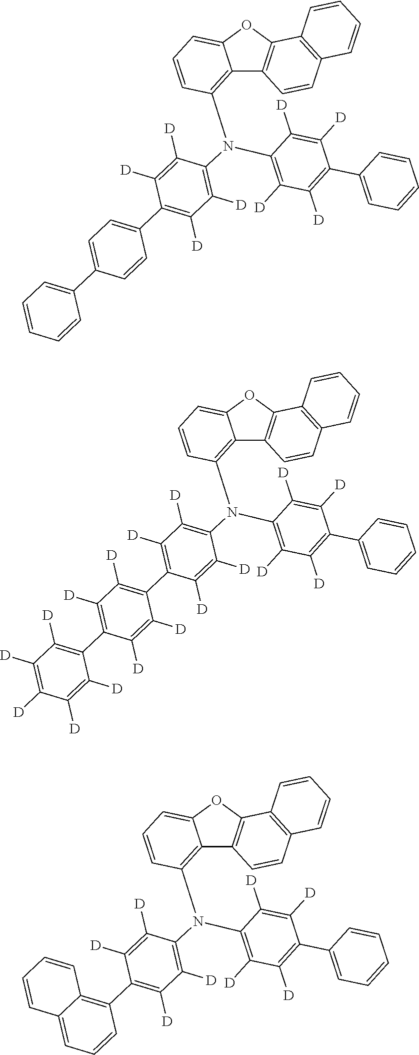 [SMILES: [2H]c1c([2H])c(N(c2c([2H])c([2H])c(-c3ccc(-c4ccccc4)cc3)c([2H])c2[2H])c2cccc3oc4c5ccccc5ccc4c23)c([2H])c([2H])c1-c1ccccc1 ; [2H]c1c([2H])c([2H])c(-c2c([2H])c([2H])c(-c3c([2H])c([2H])c(N(c4c([2H])c([2H])c(-c5ccccc5)c([2H])c4[2H])c4cccc5oc6c7ccccc7ccc6c45)c([2H])c3[2H])c([2H])c2[2H])c([2H])c1[2H] ; [2H]c1c([2H])c(N(c2c([2H])c([2H])c(-c3cccc4ccccc34)c([2H])c2[2H])c2cccc3oc4c5ccccc5ccc4c23)c([2H])c([2H])c1-c1ccccc1]

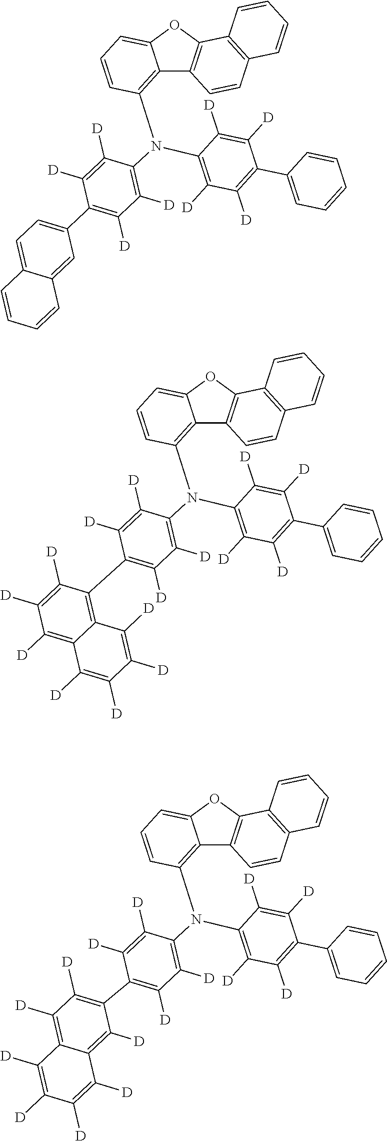 [SMILES: [2H]c1c([2H])c(N(c2c([2H])c([2H])c(-c3ccc4ccccc4c3)c([2H])c2[2H])c2cccc3oc4c5ccccc5ccc4c23)c([2H])c([2H])c1-c1ccccc1 ; [2H]c1c([2H])c(N(c2c([2H])c([2H])c(-c3c([2H])c([2H])c([2H])c4c([2H])c([2H])c([2H])c([2H])c34)c([2H])c2[2H])c2cccc3oc4c5ccccc5ccc4c23)c([2H])c([2H])c1-c1ccccc1 ; [2H]c1c([2H])c(N(c2c([2H])c([2H])c(-c3c([2H])c([2H])c4c([2H])c([2H])c([2H])c([2H])c4c3[2H])c([2H])c2[2H])c2cccc3oc4c5ccccc5ccc4c23)c([2H])c([2H])c1-c1ccccc1]

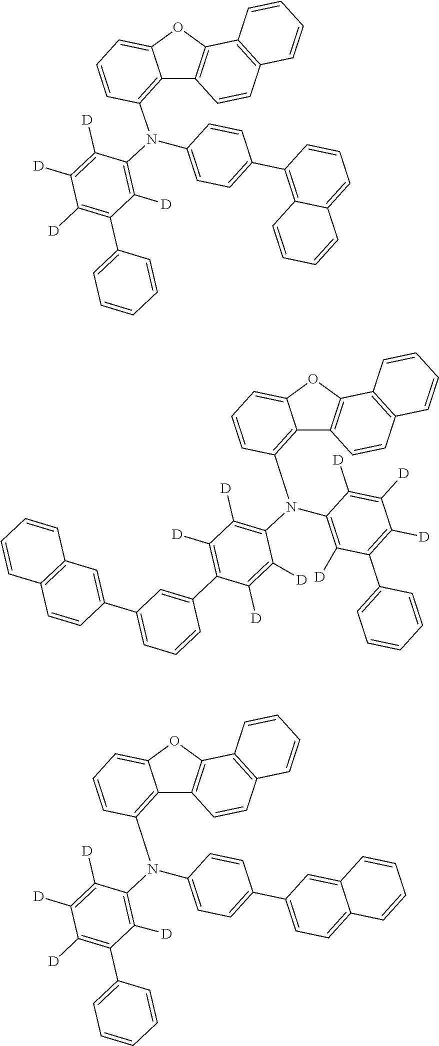 [SMILES: [2H]c1c([2H])c(-c2ccccc2)c([2H])c(N(c2ccc(-c3cccc4ccccc34)cc2)c2cccc3oc4c5ccccc5ccc4c23)c1[2H] ; [2H]c1c([2H])c(-c2ccccc2)c([2H])c(N(c2c([2H])c([2H])c(-c3cccc(-c4ccc5ccccc5c4)c3)c([2H])c2[2H])c2cccc3oc4c5ccccc5ccc4c23)c1[2H] ; [2H]c1c([2H])c(-c2ccccc2)c([2H])c(N(c2ccc(-c3ccc4ccccc4c3)cc2)c2cccc3oc4c5ccccc5ccc4c23)c1[2H]]

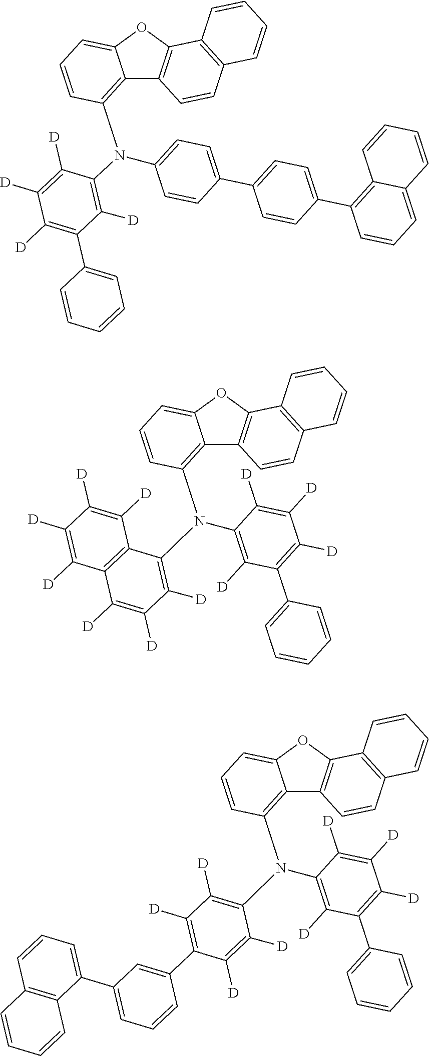 [SMILES: [2H]c1c([2H])c(-c2ccccc2)c([2H])c(N(c2ccc(-c3ccc(-c4cccc5ccccc45)cc3)cc2)c2cccc3oc4c5ccccc5ccc4c23)c1[2H] ; [2H]c1c([2H])c(-c2ccccc2)c([2H])c(N(c2c([2H])c([2H])c([2H])c3c([2H])c([2H])c([2H])c([2H])c23)c2cccc3oc4c5ccccc5ccc4c23)c1[2H] ; [2H]c1c([2H])c(-c2ccccc2)c([2H])c(N(c2c([2H])c([2H])c(-c3cccc(-c4cccc5ccccc45)c3)c([2H])c2[2H])c2cccc3oc4c5ccccc5ccc4c23)c1[2H]]

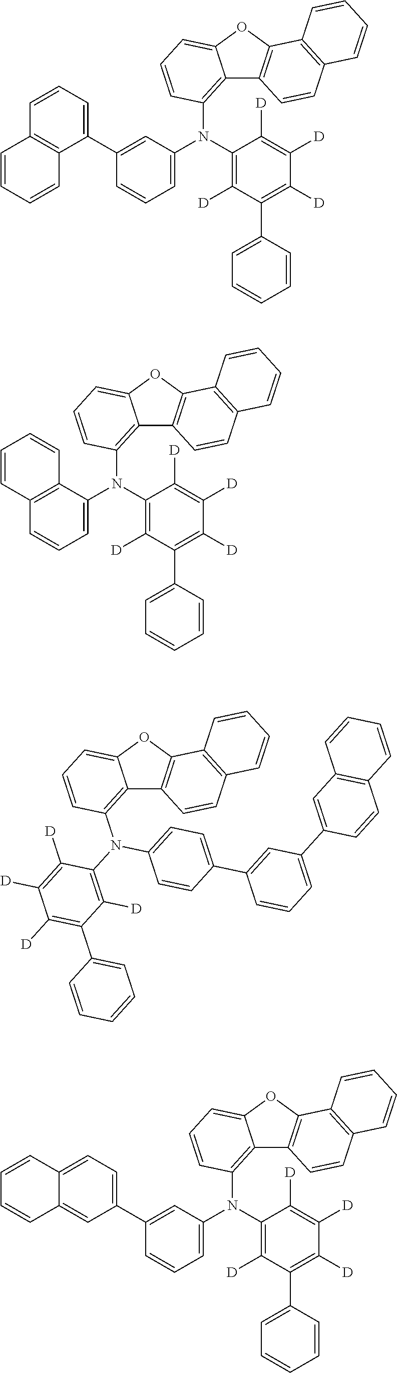

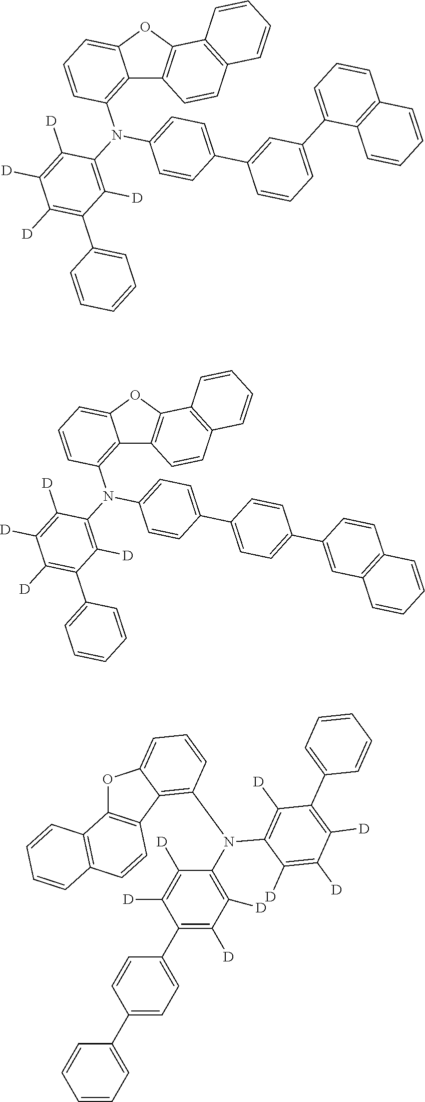 [SMILES: [2H]c1c([2H])c(-c2ccccc2)c([2H])c(N(c2ccc(-c3cccc(-c4cccc5ccccc45)c3)cc2)c2cccc3oc4c5ccccc5ccc4c23)c1[2H] ; [2H]c1c([2H])c(-c2ccccc2)c([2H])c(N(c2ccc(-c3ccc(-c4ccc5ccccc5c4)cc3)cc2)c2cccc3oc4c5ccccc5ccc4c23)c1[2H] ; [2H]c1c([2H])c(-c2ccccc2)c([2H])c(N(c2c([2H])c([2H])c(-c3ccc(-c4ccccc4)cc3)c([2H])c2[2H])c2cccc3oc4c5ccccc5ccc4c23)c1[2H]]

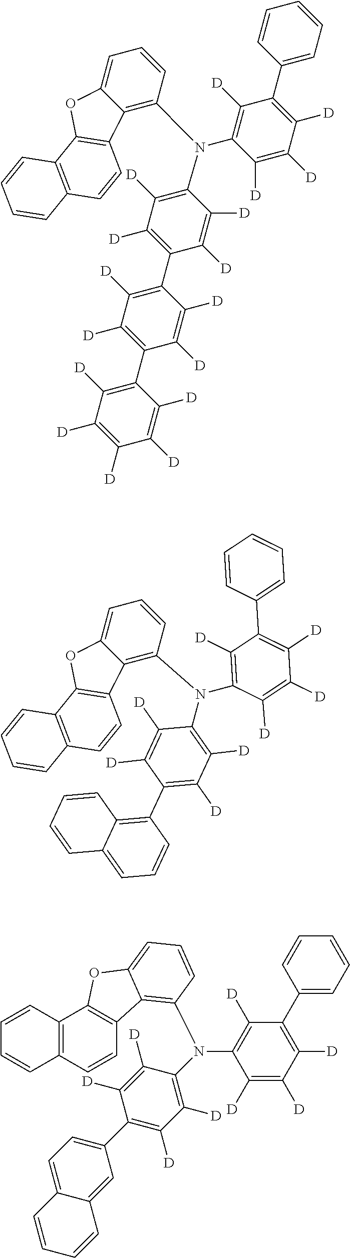 [SMILES: [2H]c1c([2H])c([2H])c(-c2c([2H])c([2H])c(-c3c([2H])c([2H])c(N(c4c([2H])c([2H])c([2H])c(-c5ccccc5)c4[2H])c4cccc5oc6c7ccccc7ccc6c45)c([2H])c3[2H])c([2H])c2[2H])c([2H])c1[2H] ; [2H]c1c([2H])c(-c2ccccc2)c([2H])c(N(c2c([2H])c([2H])c(-c3cccc4ccccc34)c([2H])c2[2H])c2cccc3oc4c5ccccc5ccc4c23)c1[2H] ; [2H]c1c([2H])c(-c2ccccc2)c([2H])c(N(c2c([2H])c([2H])c(-c3ccc4ccccc4c3)c([2H])c2[2H])c2cccc3oc4c5ccccc5ccc4c23)c1[2H]]

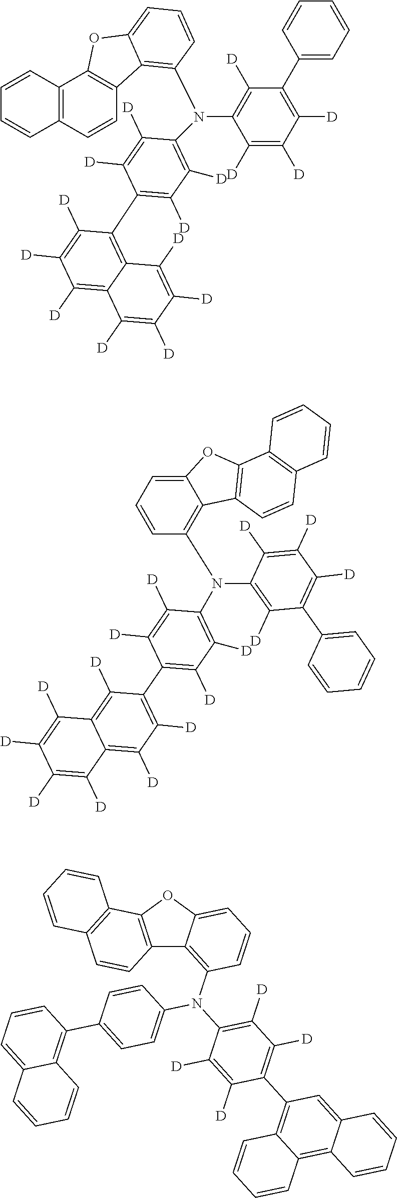 [SMILES: [2H]c1c([2H])c(-c2ccccc2)c([2H])c(N(c2c([2H])c([2H])c(-c3c([2H])c([2H])c([2H])c4c([2H])c([2H])c([2H])c([2H])c34)c([2H])c2[2H])c2cccc3oc4c5ccccc5ccc4c23)c1[2H] ; [2H]c1c([2H])c(-c2ccccc2)c([2H])c(N(c2c([2H])c([2H])c(-c3c([2H])c([2H])c4c([2H])c([2H])c([2H])c([2H])c4c3[2H])c([2H])c2[2H])c2cccc3oc4c5ccccc5ccc4c23)c1[2H] ; [2H]c1c([2H])c(N(c2ccc(-c3cccc4ccccc34)cc2)c2cccc3oc4c5ccccc5ccc4c23)c([2H])c([2H])c1-c1cc2ccccc2c2ccccc12]

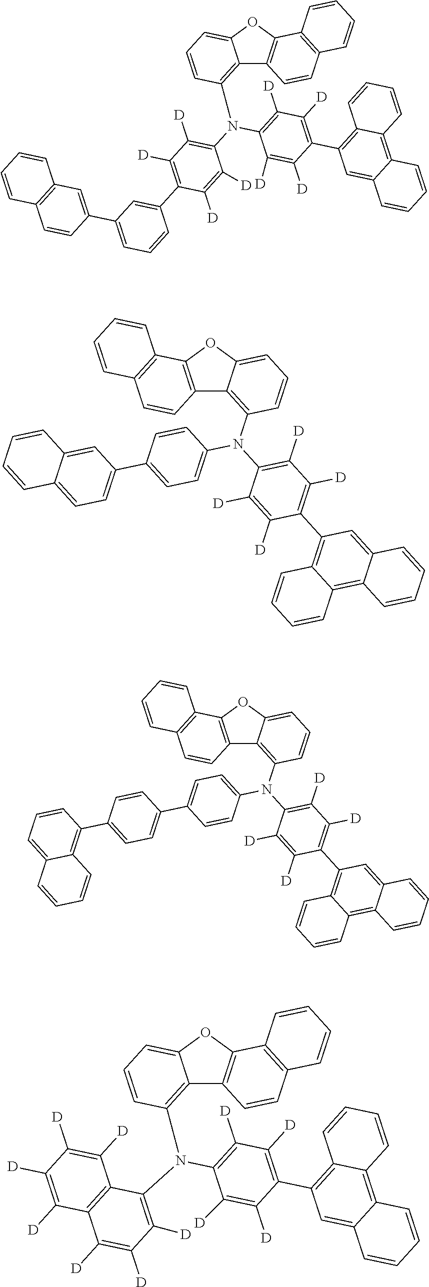

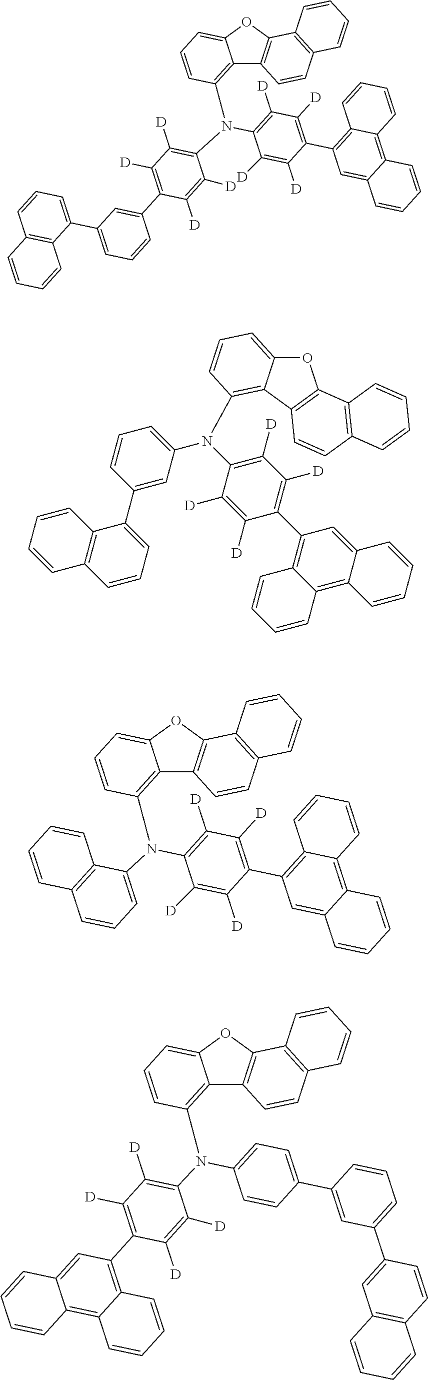 [SMILES: [2H]c1c([2H])c(N(c2c([2H])c([2H])c(-c3cc4ccccc4c4ccccc34)c([2H])c2[2H])c2cccc3oc4c5ccccc5ccc4c23)c([2H])c([2H])c1-c1cccc(-c2cccc3ccccc23)c1 ; [2H]c1c([2H])c(N(c2cccc(-c3cccc4ccccc34)c2)c2cccc3oc4c5ccccc5ccc4c23)c([2H])c([2H])c1-c1cc2ccccc2c2ccccc12 ; [2H]c1c([2H])c(N(c2cccc3ccccc23)c2cccc3oc4c5ccccc5ccc4c23)c([2H])c([2H])c1-c1cc2ccccc2c2ccccc12 ; [2H]c1c([2H])c(N(c2ccc(-c3cccc(-c4ccc5ccccc5c4)c3)cc2)c2cccc3oc4c5ccccc5ccc4c23)c([2H])c([2H])c1-c1cc2ccccc2c2ccccc12]

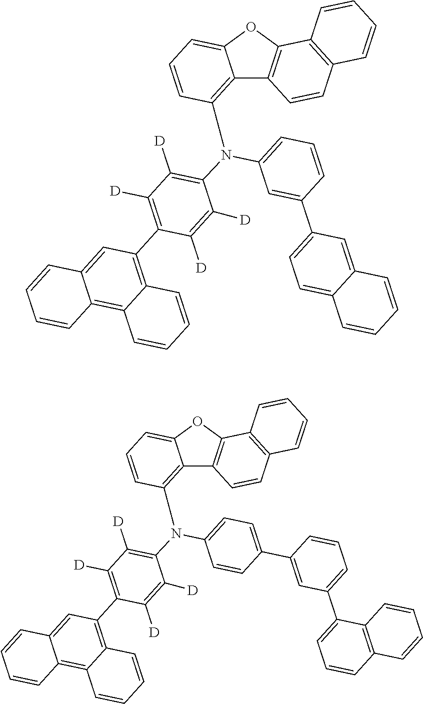 [SMILES: [2H]c1c([2H])c(N(c2cccc(-c3ccc4ccccc4c3)c2)c2cccc3oc4c5ccccc5ccc4c23)c([2H])c([2H])c1-c1cc2ccccc2c2ccccc12 ; [2H]c1c([2H])c(N(c2ccc(-c3cccc(-c4cccc5ccccc45)c3)cc2)c2cccc3oc4c5ccccc5ccc4c23)c([2H])c([2H])c1-c1cc2ccccc2c2ccccc12]

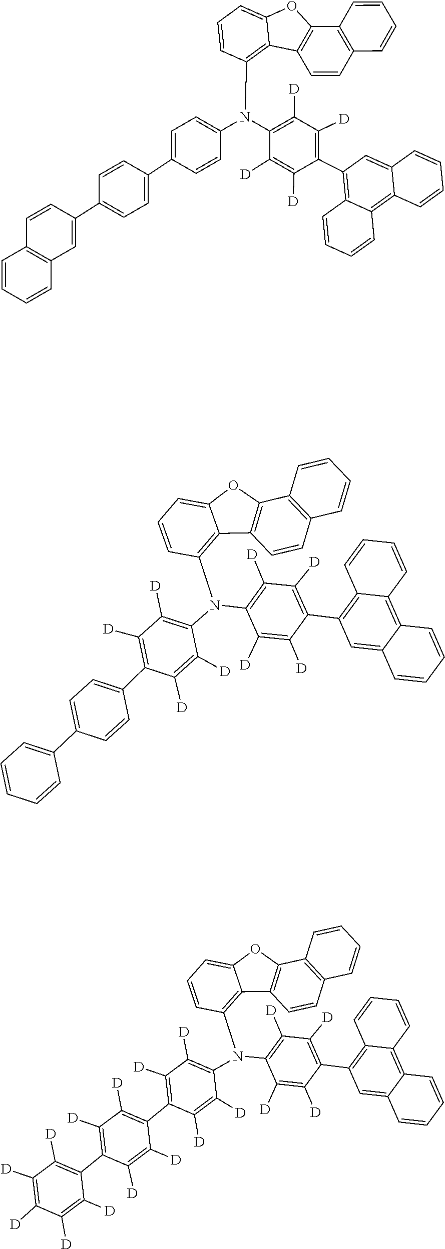 [SMILES: [2H]c1c([2H])c(N(c2ccc(-c3ccc(-c4ccc5ccccc5c4)cc3)cc2)c2cccc3oc4c5ccccc5ccc4c23)c([2H])c([2H])c1-c1cc2ccccc2c2ccccc12 ; [2H]c1c([2H])c(N(c2c([2H])c([2H])c(-c3cc4ccccc4c4ccccc34)c([2H])c2[2H])c2cccc3oc4c5ccccc5ccc4c23)c([2H])c([2H])c1-c1ccc(-c2ccccc2)cc1 ; [2H]c1c([2H])c([2H])c(-c2c([2H])c([2H])c(-c3c([2H])c([2H])c(N(c4c([2H])c([2H])c(-c5cc6ccccc6c6ccccc56)c([2H])c4[2H])c4cccc5oc6c7ccccc7ccc6c45)c([2H])c3[2H])c([2H])c2[2H])c([2H])c1[2H]]

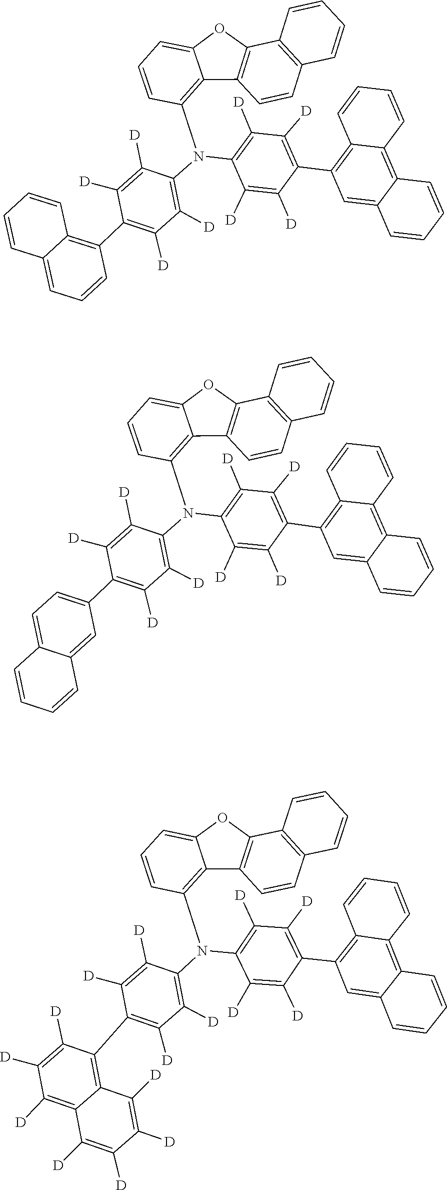 [SMILES: [2H]c1c([2H])c(N(c2c([2H])c([2H])c(-c3cc4ccccc4c4ccccc34)c([2H])c2[2H])c2cccc3oc4c5ccccc5ccc4c23)c([2H])c([2H])c1-c1cccc2ccccc12 ; [2H]c1c([2H])c(N(c2c([2H])c([2H])c(-c3cc4ccccc4c4ccccc34)c([2H])c2[2H])c2cccc3oc4c5ccccc5ccc4c23)c([2H])c([2H])c1-c1ccc2ccccc2c1 ; [2H]c1c([2H])c(N(c2c([2H])c([2H])c(-c3c([2H])c([2H])c([2H])c4c([2H])c([2H])c([2H])c([2H])c34)c([2H])c2[2H])c2cccc3oc4c5ccccc5ccc4c23)c([2H])c([2H])c1-c1cc2ccccc2c2ccccc12]

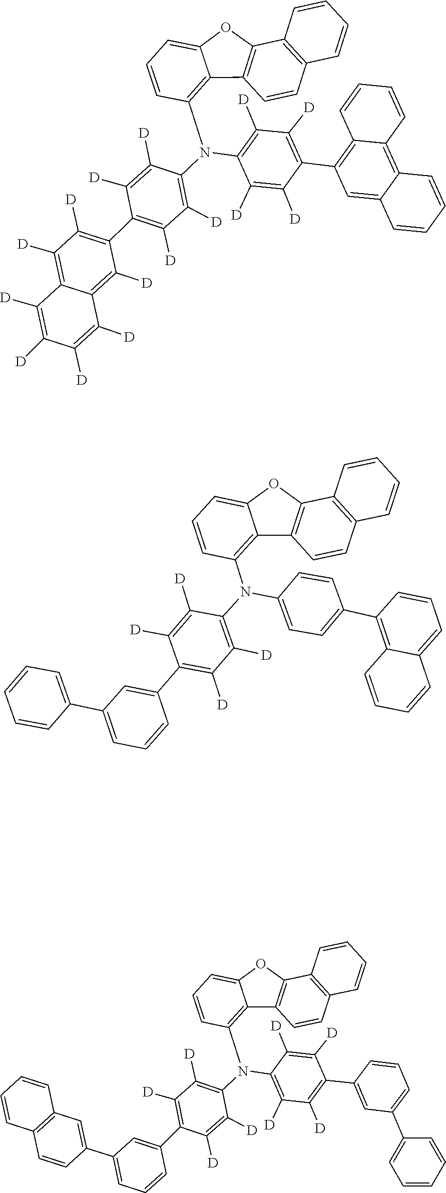 [SMILES: [2H]c1c([2H])c(N(c2c([2H])c([2H])c(-c3cc4ccccc4c4ccccc34)c([2H])c2[2H])c2cccc3oc4c5ccccc5ccc4c23)c([2H])c([2H])c1-c1c([2H])c([2H])c2c([2H])c([2H])c([2H])c([2H])c2c1[2H] ; [2H]c1c([2H])c(N(c2ccc(-c3cccc4ccccc34)cc2)c2cccc3oc4c5ccccc5ccc4c23)c([2H])c([2H])c1-c1cccc(-c2ccccc2)c1 ; [2H]c1c([2H])c(N(c2c([2H])c([2H])c(-c3cccc(-c4ccc5ccccc5c4)c3)c([2H])c2[2H])c2cccc3oc4c5ccccc5ccc4c23)c([2H])c([2H])c1-c1cccc(-c2ccccc2)c1]

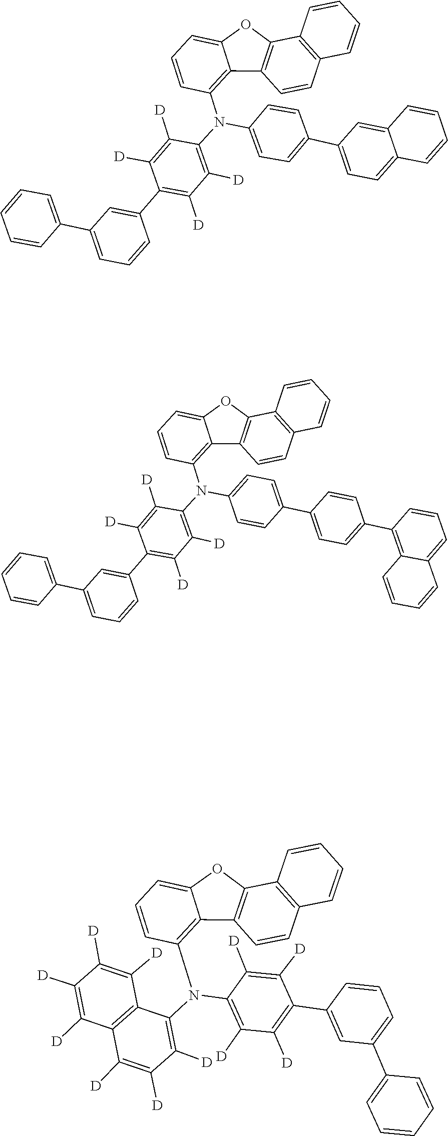 [SMILES: [2H]c1c([2H])c(N(c2ccc(-c3ccc4ccccc4c3)cc2)c2cccc3oc4c5ccccc5ccc4c23)c([2H])c([2H])c1-c1cccc(-c2ccccc2)c1 ; [2H]c1c([2H])c(N(c2ccc(-c3ccc(-c4cccc5ccccc45)cc3)cc2)c2cccc3oc4c5ccccc5ccc4c23)c([2H])c([2H])c1-c1cccc(-c2ccccc2)c1 ; [2H]c1c([2H])c(N(c2c([2H])c([2H])c([2H])c3c([2H])c([2H])c([2H])c([2H])c23)c2cccc3oc4c5ccccc5ccc4c23)c([2H])c([2H])c1-c1cccc(-c2ccccc2)c1]

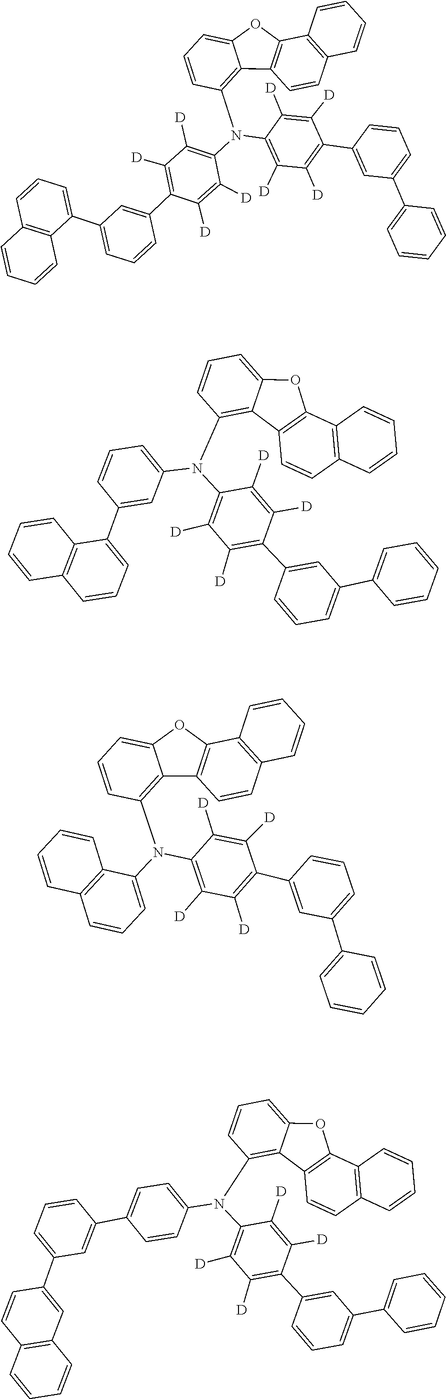 [SMILES: [2H]c1c([2H])c(N(c2c([2H])c([2H])c(-c3cccc(-c4cccc5ccccc45)c3)c([2H])c2[2H])c2cccc3oc4c5ccccc5ccc4c23)c([2H])c([2H])c1-c1cccc(-c2ccccc2)c1 ; [2H]c1c([2H])c(N(c2cccc(-c3cccc4ccccc34)c2)c2cccc3oc4c5ccccc5ccc4c23)c([2H])c([2H])c1-c1cccc(-c2ccccc2)c1 ; [2H]c1c([2H])c(N(c2cccc3ccccc23)c2cccc3oc4c5ccccc5ccc4c23)c([2H])c([2H])c1-c1cccc(-c2ccccc2)c1 ; [2H]c1c([2H])c(N(c2ccc(-c3cccc(-c4ccc5ccccc5c4)c3)cc2)c2cccc3oc4c5ccccc5ccc4c23)c([2H])c([2H])c1-c1cccc(-c2ccccc2)c1]

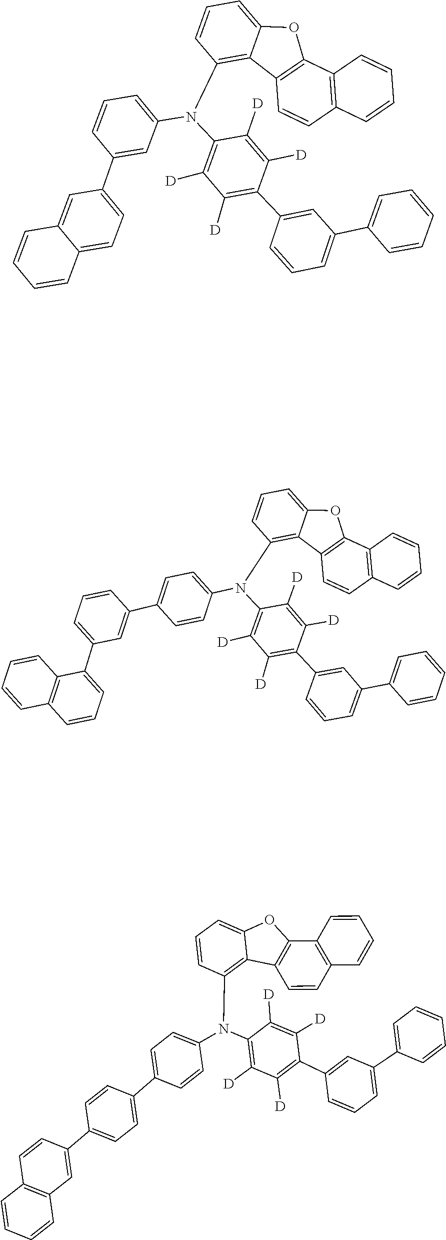 [SMILES: [2H]c1c([2H])c(N(c2cccc(-c3ccc4ccccc4c3)c2)c2cccc3oc4c5ccccc5ccc4c23)c([2H])c([2H])c1-c1cccc(-c2ccccc2)c1 ; [2H]c1c([2H])c(N(c2ccc(-c3cccc(-c4cccc5ccccc45)c3)cc2)c2cccc3oc4c5ccccc5ccc4c23)c([2H])c([2H])c1-c1cccc(-c2ccccc2)c1 ; [2H]c1c([2H])c(N(c2ccc(-c3ccc(-c4ccc5ccccc5c4)cc3)cc2)c2cccc3oc4c5ccccc5ccc4c23)c([2H])c([2H])c1-c1cccc(-c2ccccc2)c1]

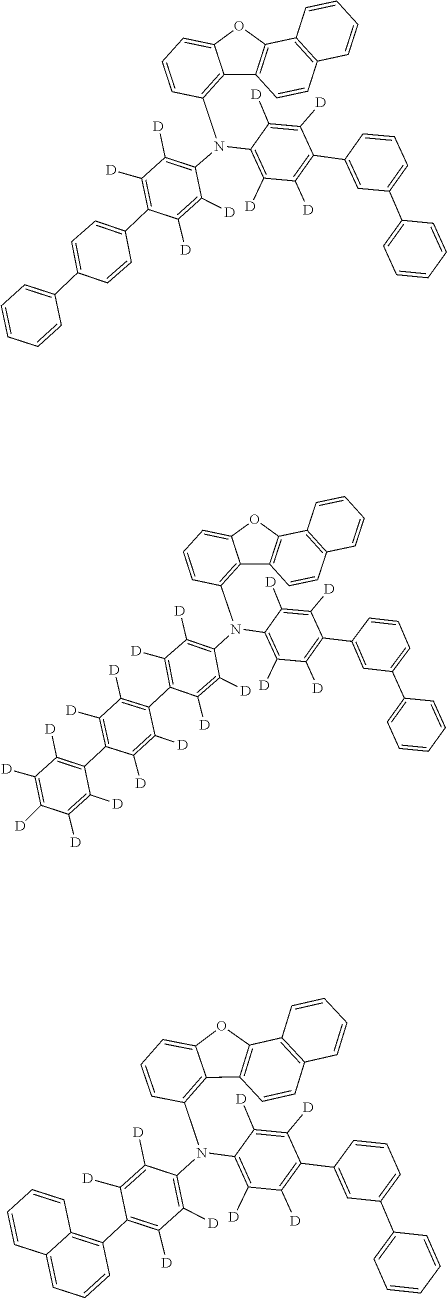 [SMILES: [2H]c1c([2H])c(N(c2c([2H])c([2H])c(-c3cccc(-c4ccccc4)c3)c([2H])c2[2H])c2cccc3oc4c5ccccc5ccc4c23)c([2H])c([2H])c1-c1ccc(-c2ccccc2)cc1 ; [2H]c1c([2H])c([2H])c(-c2c([2H])c([2H])c(-c3c([2H])c([2H])c(N(c4c([2H])c([2H])c(-c5cccc(-c6ccccc6)c5)c([2H])c4[2H])c4cccc5oc6c7ccccc7ccc6c45)c([2H])c3[2H])c([2H])c2[2H])c([2H])c1[2H] ; [2H]c1c([2H])c(N(c2c([2H])c([2H])c(-c3cccc4ccccc34)c([2H])c2[2H])c2cccc3oc4c5ccccc5ccc4c23)c([2H])c([2H])c1-c1cccc(-c2ccccc2)c1]

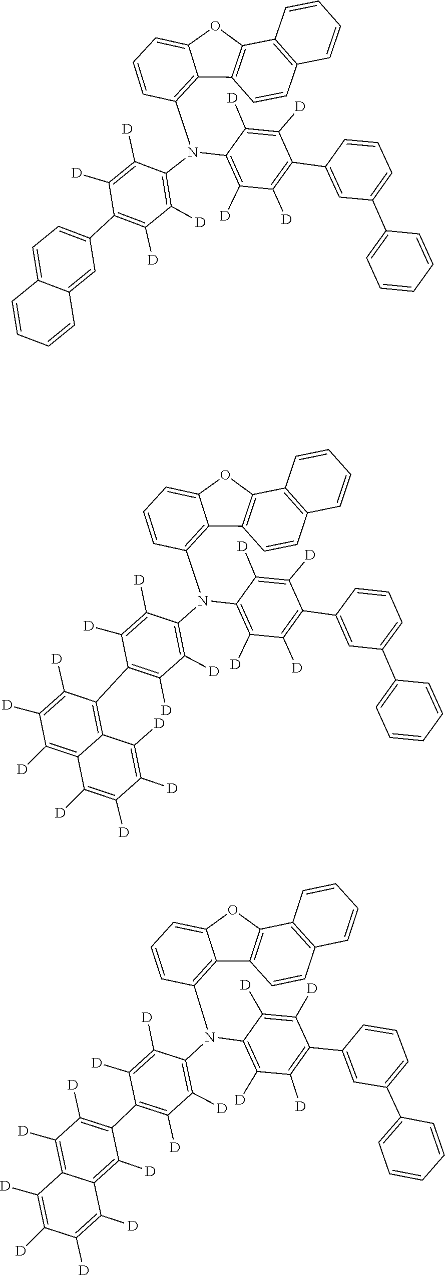 [SMILES: [2H]c1c([2H])c(N(c2c([2H])c([2H])c(-c3ccc4ccccc4c3)c([2H])c2[2H])c2cccc3oc4c5ccccc5ccc4c23)c([2H])c([2H])c1-c1cccc(-c2ccccc2)c1 ; [2H]c1c([2H])c(N(c2c([2H])c([2H])c(-c3c([2H])c([2H])c([2H])c4c([2H])c([2H])c([2H])c([2H])c34)c([2H])c2[2H])c2cccc3oc4c5ccccc5ccc4c23)c([2H])c([2H])c1-c1cccc(-c2ccccc2)c1 ; [2H]c1c([2H])c(N(c2c([2H])c([2H])c(-c3c([2H])c([2H])c4c([2H])c([2H])c([2H])c([2H])c4c3[2H])c([2H])c2[2H])c2cccc3oc4c5ccccc5ccc4c23)c([2H])c([2H])c1-c1cccc(-c2ccccc2)c1]

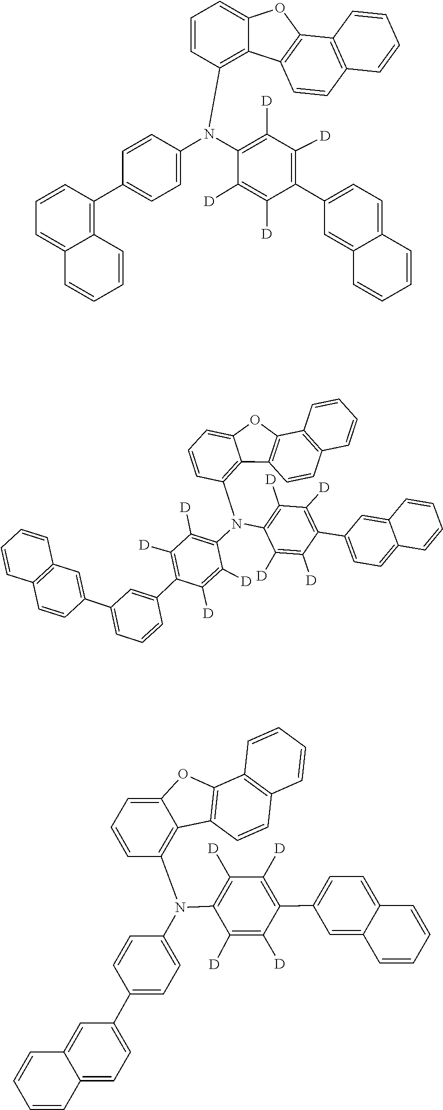 [SMILES: [2H]c1c([2H])c(N(c2ccc(-c3cccc4ccccc34)cc2)c2cccc3oc4c5ccccc5ccc4c23)c([2H])c([2H])c1-c1ccc2ccccc2c1 ; [2H]c1c([2H])c(N(c2c([2H])c([2H])c(-c3ccc4ccccc4c3)c([2H])c2[2H])c2cccc3oc4c5ccccc5ccc4c23)c([2H])c([2H])c1-c1cccc(-c2ccc3ccccc3c2)c1 ; [2H]c1c([2H])c(N(c2ccc(-c3ccc4ccccc4c3)cc2)c2cccc3oc4c5ccccc5ccc4c23)c([2H])c([2H])c1-c1ccc2ccccc2c1]

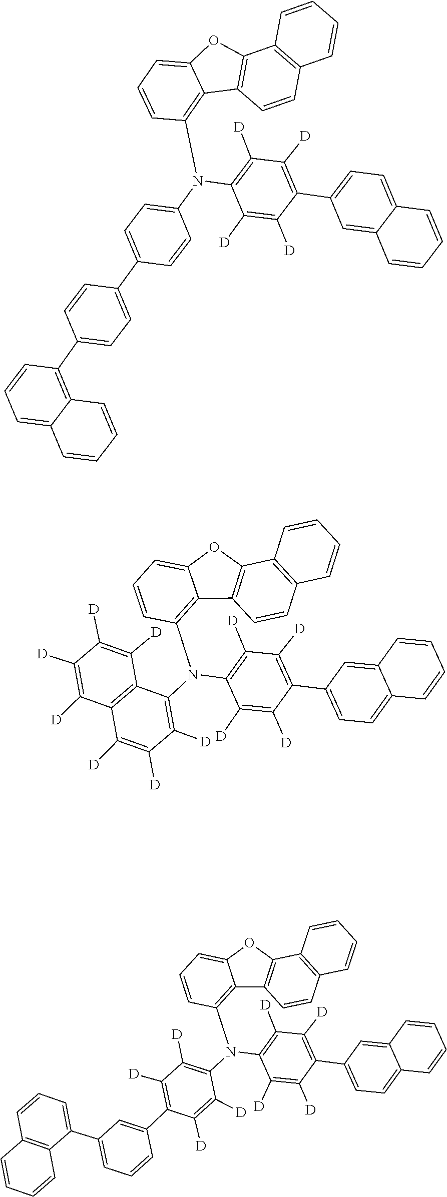 [SMILES: [2H]c1c([2H])c(N(c2ccc(-c3ccc(-c4cccc5ccccc45)cc3)cc2)c2cccc3oc4c5ccccc5ccc4c23)c([2H])c([2H])c1-c1ccc2ccccc2c1 ; [2H]c1c([2H])c(N(c2c([2H])c([2H])c([2H])c3c([2H])c([2H])c([2H])c([2H])c23)c2cccc3oc4c5ccccc5ccc4c23)c([2H])c([2H])c1-c1ccc2ccccc2c1 ; [2H]c1c([2H])c(N(c2c([2H])c([2H])c(-c3ccc4ccccc4c3)c([2H])c2[2H])c2cccc3oc4c5ccccc5ccc4c23)c([2H])c([2H])c1-c1cccc(-c2cccc3ccccc23)c1]

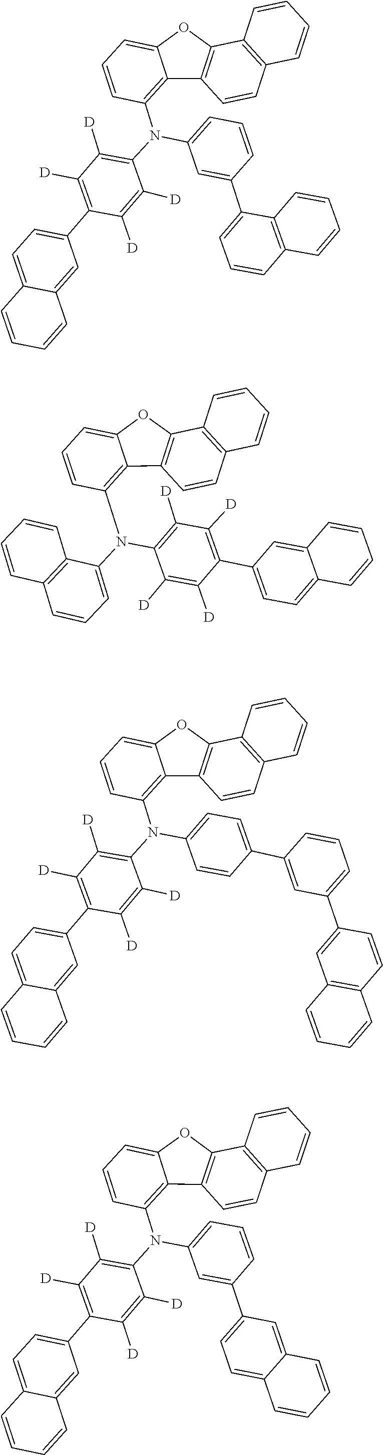 [SMILES: [2H]c1c([2H])c(N(c2cccc(-c3cccc4ccccc34)c2)c2cccc3oc4c5ccccc5ccc4c23)c([2H])c([2H])c1-c1ccc2ccccc2c1 ; [2H]c1c([2H])c(N(c2cccc3ccccc23)c2cccc3oc4c5ccccc5ccc4c23)c([2H])c([2H])c1-c1ccc2ccccc2c1 ; [2H]c1c([2H])c(N(c2ccc(-c3cccc(-c4ccc5ccccc5c4)c3)cc2)c2cccc3oc4c5ccccc5ccc4c23)c([2H])c([2H])c1-c1ccc2ccccc2c1 ; [2H]c1c([2H])c(N(c2cccc(-c3ccc4ccccc4c3)c2)c2cccc3oc4c5ccccc5ccc4c23)c([2H])c([2H])c1-c1ccc2ccccc2c1]

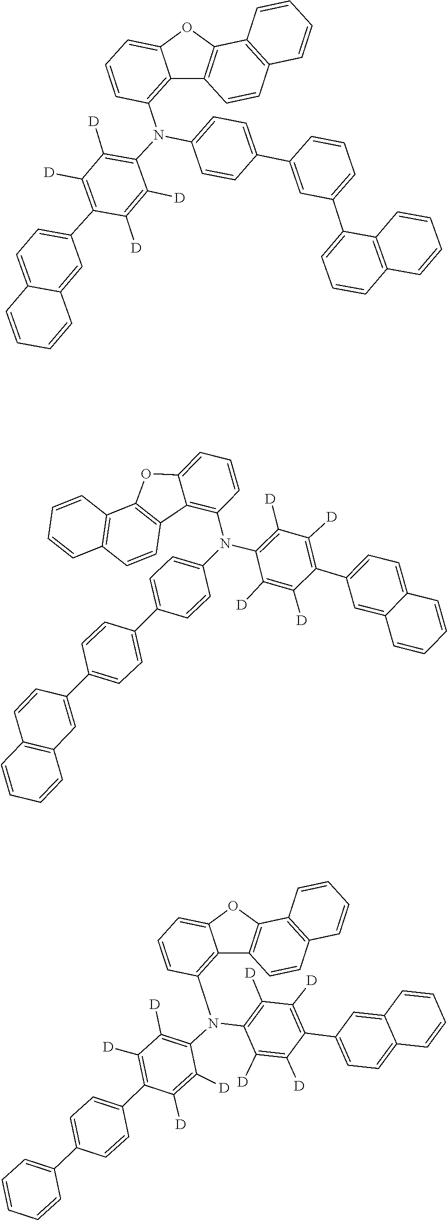 [SMILES: [2H]c1c([2H])c(N(c2ccc(-c3cccc(-c4cccc5ccccc45)c3)cc2)c2cccc3oc4c5ccccc5ccc4c23)c([2H])c([2H])c1-c1ccc2ccccc2c1 ; [2H]c1c([2H])c(N(c2ccc(-c3ccc(-c4ccc5ccccc5c4)cc3)cc2)c2cccc3oc4c5ccccc5ccc4c23)c([2H])c([2H])c1-c1ccc2ccccc2c1 ; [2H]c1c([2H])c(N(c2c([2H])c([2H])c(-c3ccc4ccccc4c3)c([2H])c2[2H])c2cccc3oc4c5ccccc5ccc4c23)c([2H])c([2H])c1-c1ccc(-c2ccccc2)cc1]

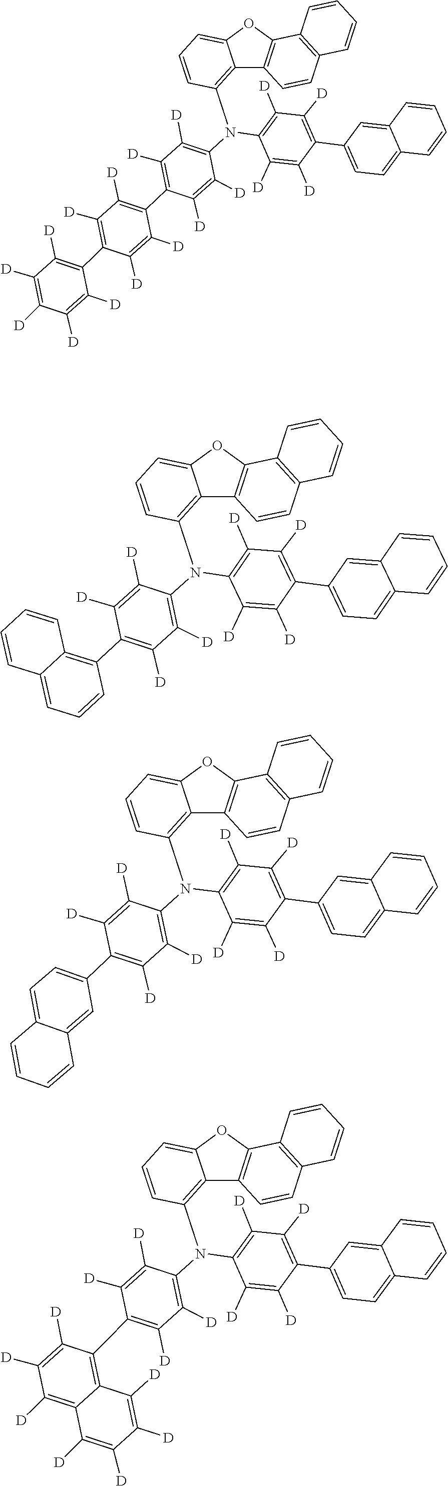

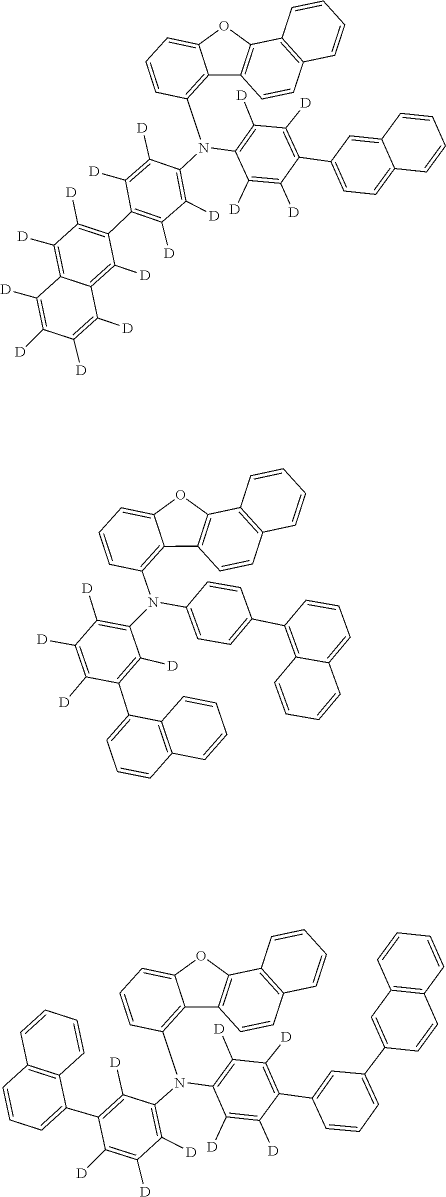 [SMILES: [2H]c1c([2H])c(N(c2c([2H])c([2H])c(-c3c([2H])c([2H])c4c([2H])c([2H])c([2H])c([2H])c4c3[2H])c([2H])c2[2H])c2cccc3oc4c5ccccc5ccc4c23)c([2H])c([2H])c1-c1ccc2ccccc2c1 ; [2H]c1c([2H])c(-c2cccc3ccccc23)c([2H])c(N(c2ccc(-c3cccc4ccccc34)cc2)c2cccc3oc4c5ccccc5ccc4c23)c1[2H] ; [2H]c1c([2H])c(-c2cccc3ccccc23)c([2H])c(N(c2c([2H])c([2H])c(-c3cccc(-c4ccc5ccccc5c4)c3)c([2H])c2[2H])c2cccc3oc4c5ccccc5ccc4c23)c1[2H]]

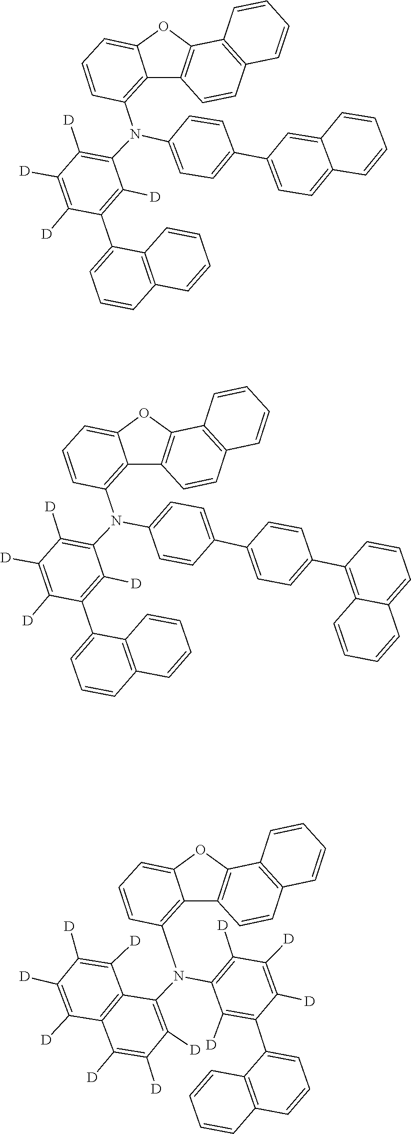 [SMILES: [2H]c1c([2H])c(-c2cccc3ccccc23)c([2H])c(N(c2ccc(-c3ccc4ccccc4c3)cc2)c2cccc3oc4c5ccccc5ccc4c23)c1[2H] ; [2H]c1c([2H])c(-c2cccc3ccccc23)c([2H])c(N(c2ccc(-c3ccc(-c4cccc5ccccc45)cc3)cc2)c2cccc3oc4c5ccccc5ccc4c23)c1[2H] ; [2H]c1c([2H])c(-c2cccc3ccccc23)c([2H])c(N(c2c([2H])c([2H])c([2H])c3c([2H])c([2H])c([2H])c([2H])c23)c2cccc3oc4c5ccccc5ccc4c23)c1[2H]]

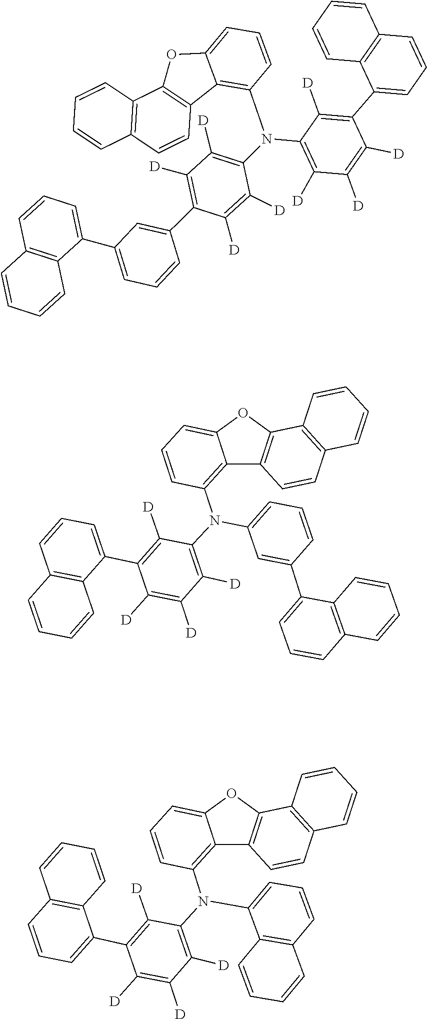 [SMILES: [2H]c1c([2H])c(-c2cccc3ccccc23)c([2H])c(N(c2c([2H])c([2H])c(-c3cccc(-c4cccc5ccccc45)c3)c([2H])c2[2H])c2cccc3oc4c5ccccc5ccc4c23)c1[2H] ; [2H]c1c([2H])c(-c2cccc3ccccc23)c([2H])c(N(c2cccc(-c3cccc4ccccc34)c2)c2cccc3oc4c5ccccc5ccc4c23)c1[2H] ; [2H]c1c([2H])c(-c2cccc3ccccc23)c([2H])c(N(c2cccc3ccccc23)c2cccc3oc4c5ccccc5ccc4c23)c1[2H]]

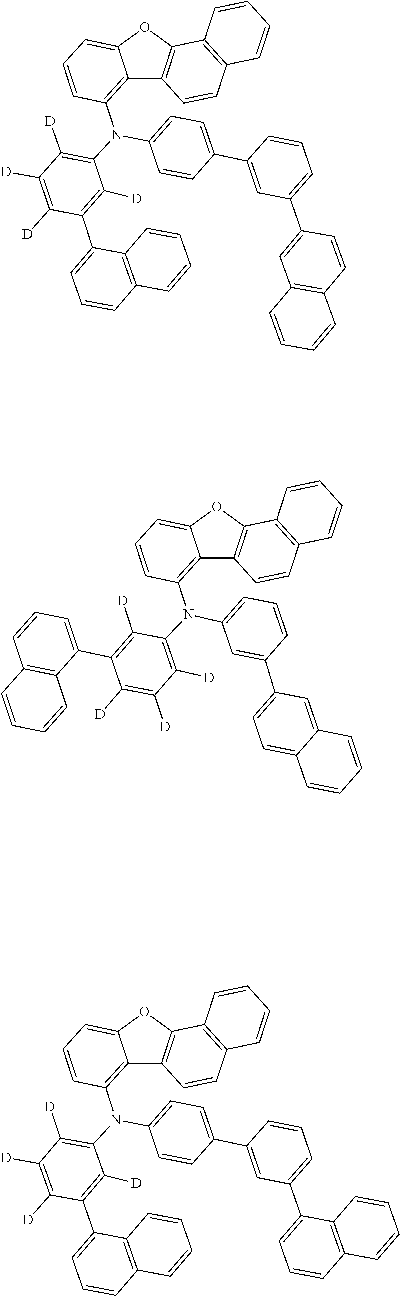 [SMILES: [2H]c1c([2H])c(-c2cccc3ccccc23)c([2H])c(N(c2ccc(-c3cccc(-c4ccc5ccccc5c4)c3)cc2)c2cccc3oc4c5ccccc5ccc4c23)c1[2H] ; [2H]c1c([2H])c(-c2cccc3ccccc23)c([2H])c(N(c2cccc(-c3ccc4ccccc4c3)c2)c2cccc3oc4c5ccccc5ccc4c23)c1[2H] ; [2H]c1c([2H])c(-c2cccc3ccccc23)c([2H])c(N(c2ccc(-c3cccc(-c4cccc5ccccc45)c3)cc2)c2cccc3oc4c5ccccc5ccc4c23)c1[2H]]

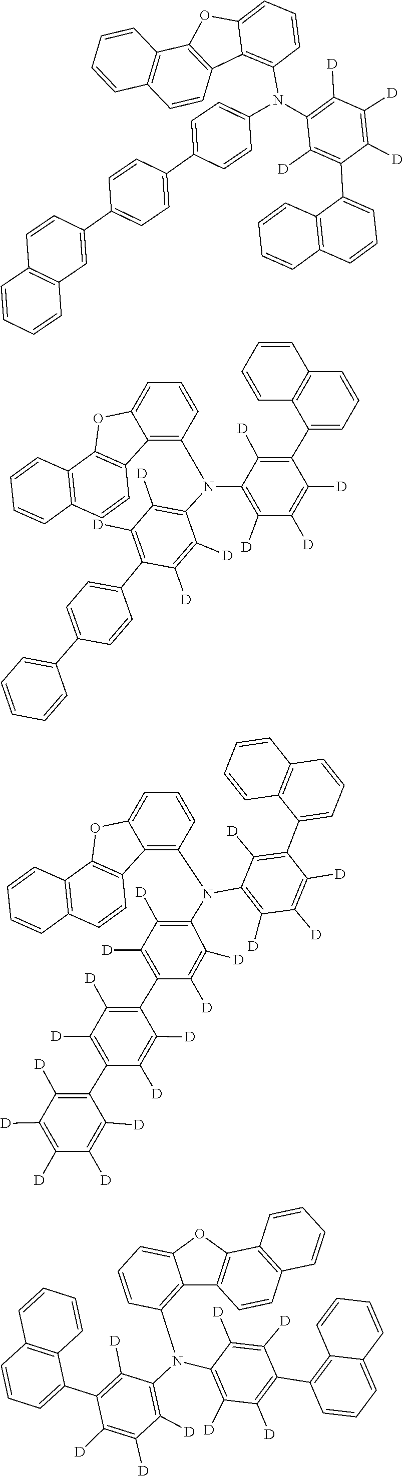

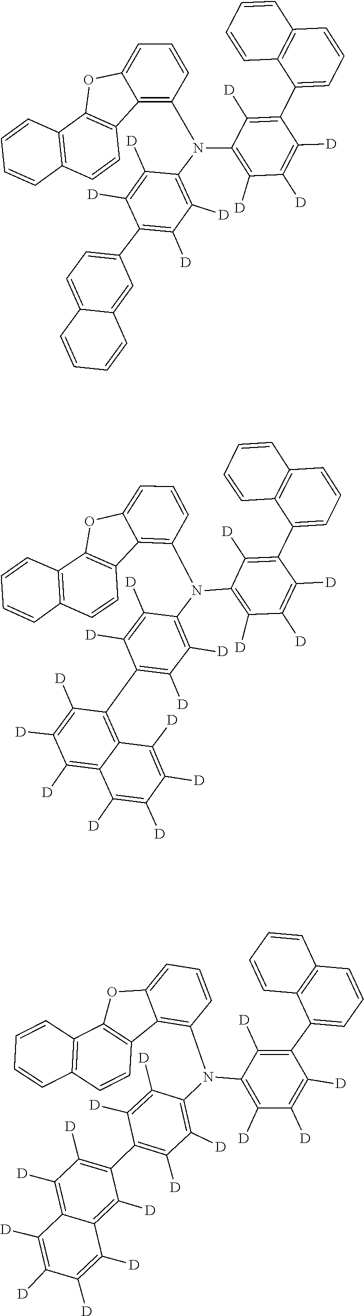 [SMILES: [2H]c1c([2H])c(-c2cccc3ccccc23)c([2H])c(N(c2c([2H])c([2H])c(-c3ccc4ccccc4c3)c([2H])c2[2H])c2cccc3oc4c5ccccc5ccc4c23)c1[2H] ; [2H]c1c([2H])c(-c2cccc3ccccc23)c([2H])c(N(c2c([2H])c([2H])c(-c3c([2H])c([2H])c([2H])c4c([2H])c([2H])c([2H])c([2H])c34)c([2H])c2[2H])c2cccc3oc4c5ccccc5ccc4c23)c1[2H] ; [2H]c1c([2H])c(-c2cccc3ccccc23)c([2H])c(N(c2c([2H])c([2H])c(-c3c([2H])c([2H])c4c([2H])c([2H])c([2H])c([2H])c4c3[2H])c([2H])c2[2H])c2cccc3oc4c5ccccc5ccc4c23)c1[2H]]

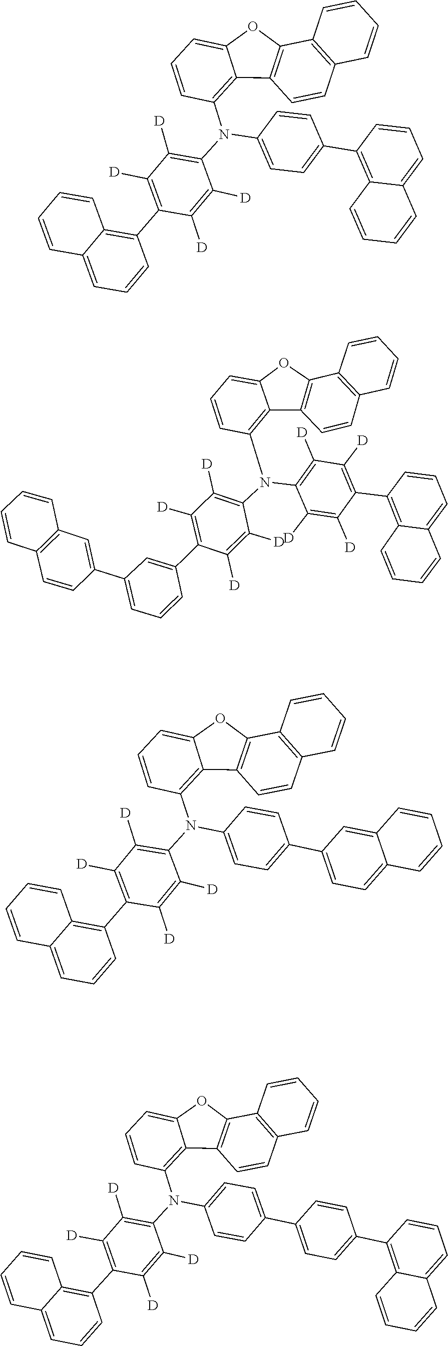 [SMILES: [2H]c1c([2H])c(N(c2ccc(-c3cccc4ccccc34)cc2)c2cccc3oc4c5ccccc5ccc4c23)c([2H])c([2H])c1-c1cccc2ccccc12 ; [2H]c1c([2H])c(N(c2c([2H])c([2H])c(-c3cccc4ccccc34)c([2H])c2[2H])c2cccc3oc4c5ccccc5ccc4c23)c([2H])c([2H])c1-c1cccc(-c2ccc3ccccc3c2)c1 ; [2H]c1c([2H])c(N(c2ccc(-c3ccc4ccccc4c3)cc2)c2cccc3oc4c5ccccc5ccc4c23)c([2H])c([2H])c1-c1cccc2ccccc12 ; [2H]c1c([2H])c(N(c2ccc(-c3ccc(-c4cccc5ccccc45)cc3)cc2)c2cccc3oc4c5ccccc5ccc4c23)c([2H])c([2H])c1-c1cccc2ccccc12]

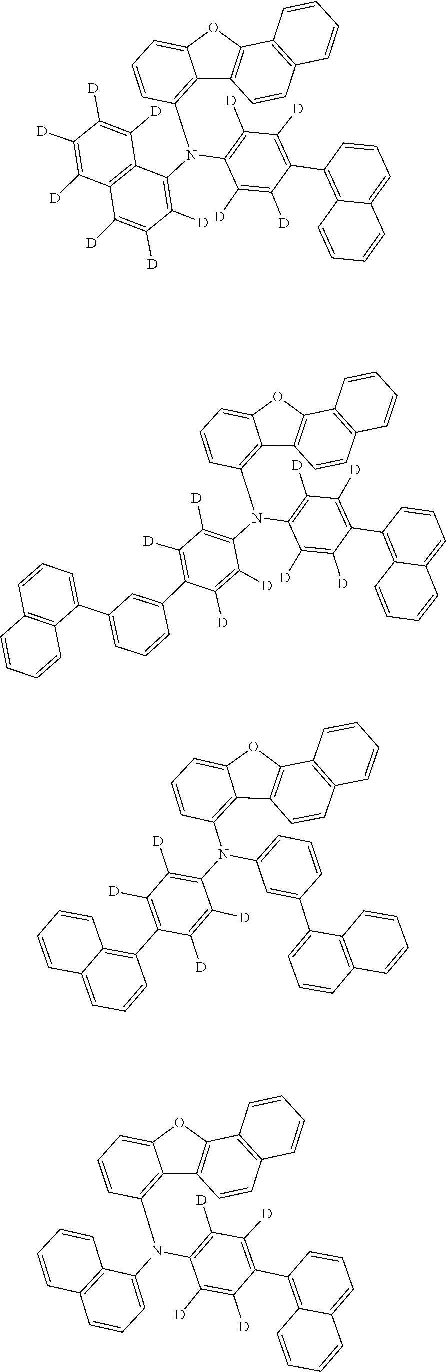 [SMILES: [2H]c1c([2H])c(N(c2c([2H])c([2H])c([2H])c3c([2H])c([2H])c([2H])c([2H])c23)c2cccc3oc4c5ccccc5ccc4c23)c([2H])c([2H])c1-c1cccc2ccccc12 ; [2H]c1c([2H])c(N(c2c([2H])c([2H])c(-c3cccc4ccccc34)c([2H])c2[2H])c2cccc3oc4c5ccccc5ccc4c23)c([2H])c([2H])c1-c1cccc(-c2cccc3ccccc23)c1 ; [2H]c1c([2H])c(N(c2cccc(-c3cccc4ccccc34)c2)c2cccc3oc4c5ccccc5ccc4c23)c([2H])c([2H])c1-c1cccc2ccccc12 ; [2H]c1c([2H])c(N(c2cccc3ccccc23)c2cccc3oc4c5ccccc5ccc4c23)c([2H])c([2H])c1-c1cccc2ccccc12]

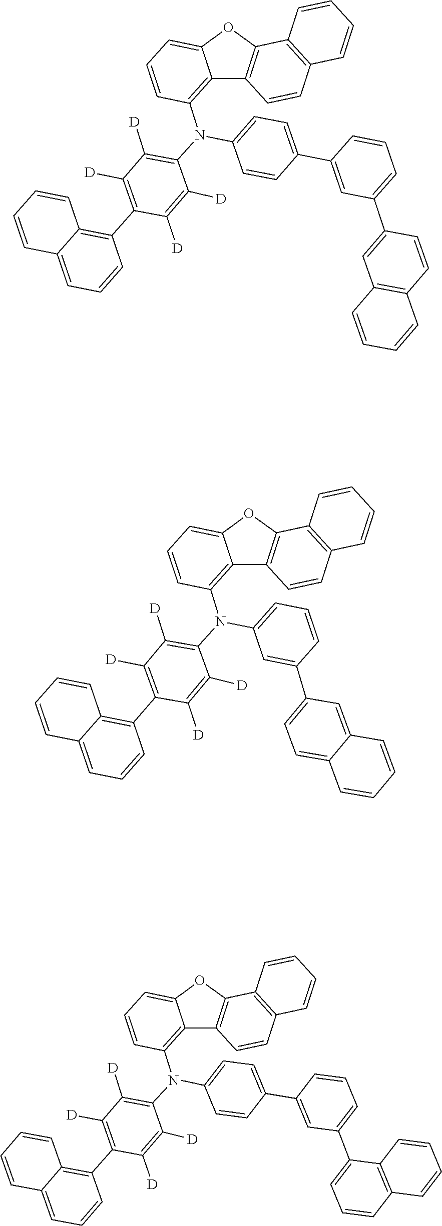 [SMILES: [2H]c1c([2H])c(N(c2ccc(-c3cccc(-c4ccc5ccccc5c4)c3)cc2)c2cccc3oc4c5ccccc5ccc4c23)c([2H])c([2H])c1-c1cccc2ccccc12 ; [2H]c1c([2H])c(N(c2cccc(-c3ccc4ccccc4c3)c2)c2cccc3oc4c5ccccc5ccc4c23)c([2H])c([2H])c1-c1cccc2ccccc12 ; [2H]c1c([2H])c(N(c2ccc(-c3cccc(-c4cccc5ccccc45)c3)cc2)c2cccc3oc4c5ccccc5ccc4c23)c([2H])c([2H])c1-c1cccc2ccccc12]

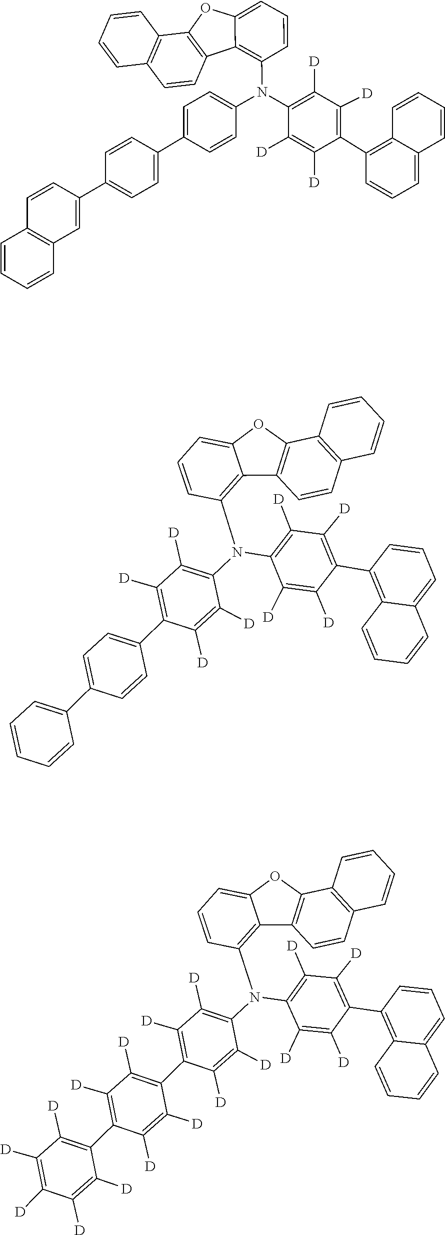 [SMILES: [2H]c1c([2H])c(N(c2ccc(-c3ccc(-c4ccc5ccccc5c4)cc3)cc2)c2cccc3oc4c5ccccc5ccc4c23)c([2H])c([2H])c1-c1cccc2ccccc12 ; [2H]c1c([2H])c(N(c2c([2H])c([2H])c(-c3cccc4ccccc34)c([2H])c2[2H])c2cccc3oc4c5ccccc5ccc4c23)c([2H])c([2H])c1-c1ccc(-c2ccccc2)cc1 ; [2H]c1c([2H])c([2H])c(-c2c([2H])c([2H])c(-c3c([2H])c([2H])c(N(c4c([2H])c([2H])c(-c5cccc6ccccc56)c([2H])c4[2H])c4cccc5oc6c7ccccc7ccc6c45)c([2H])c3[2H])c([2H])c2[2H])c([2H])c1[2H]]

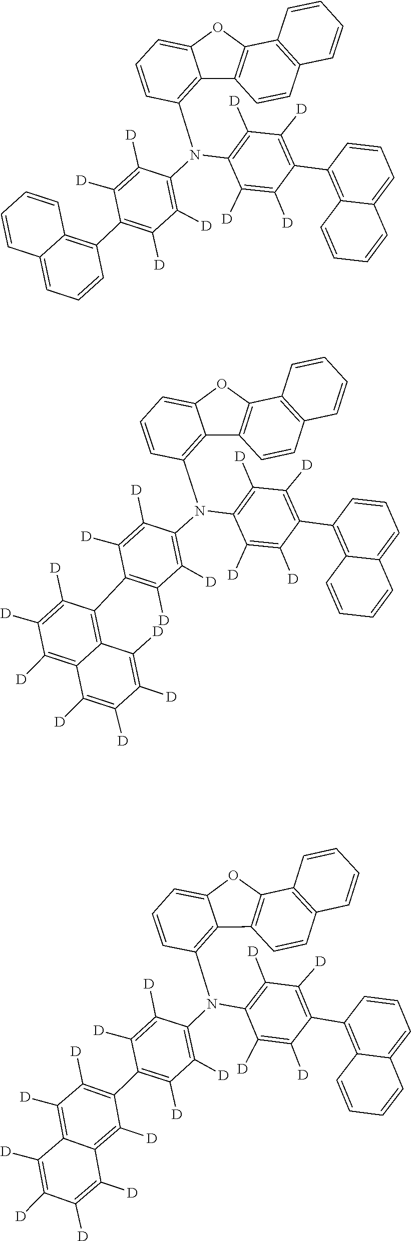 [SMILES: [2H]c1c([2H])c(N(c2c([2H])c([2H])c(-c3cccc4ccccc34)c([2H])c2[2H])c2cccc3oc4c5ccccc5ccc4c23)c([2H])c([2H])c1-c1cccc2ccccc12 ; [2H]c1c([2H])c(N(c2c([2H])c([2H])c(-c3c([2H])c([2H])c([2H])c4c([2H])c([2H])c([2H])c([2H])c34)c([2H])c2[2H])c2cccc3oc4c5ccccc5ccc4c23)c([2H])c([2H])c1-c1cccc2ccccc12 ; [2H]c1c([2H])c(N(c2c([2H])c([2H])c(-c3cccc4ccccc34)c([2H])c2[2H])c2cccc3oc4c5ccccc5ccc4c23)c([2H])c([2H])c1-c1c([2H])c([2H])c2c([2H])c([2H])c([2H])c([2H])c2c1[2H]]

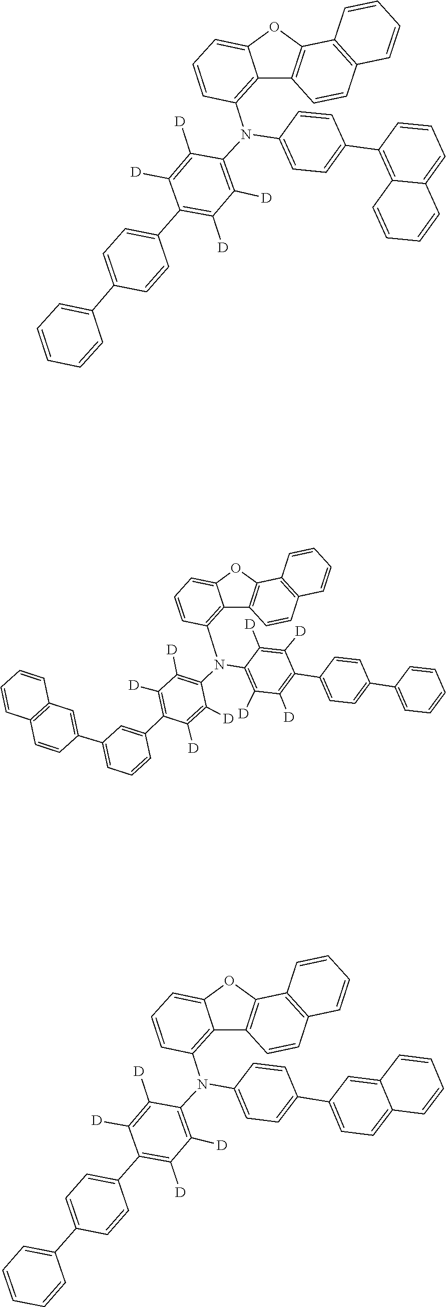 [SMILES: [2H]c1c([2H])c(N(c2ccc(-c3cccc4ccccc34)cc2)c2cccc3oc4c5ccccc5ccc4c23)c([2H])c([2H])c1-c1ccc(-c2ccccc2)cc1 ; [2H]c1c([2H])c(N(c2c([2H])c([2H])c(-c3cccc(-c4ccc5ccccc5c4)c3)c([2H])c2[2H])c2cccc3oc4c5ccccc5ccc4c23)c([2H])c([2H])c1-c1ccc(-c2ccccc2)cc1 ; [2H]c1c([2H])c(N(c2ccc(-c3ccc4ccccc4c3)cc2)c2cccc3oc4c5ccccc5ccc4c23)c([2H])c([2H])c1-c1ccc(-c2ccccc2)cc1]

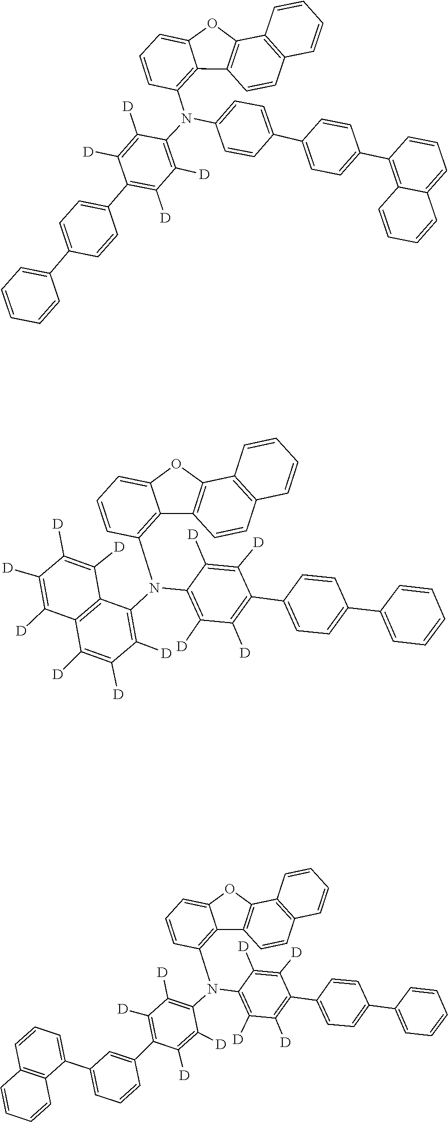 [SMILES: [2H]c1c([2H])c(N(c2ccc(-c3ccc(-c4cccc5ccccc45)cc3)cc2)c2cccc3oc4c5ccccc5ccc4c23)c([2H])c([2H])c1-c1ccc(-c2ccccc2)cc1 ; [2H]c1c([2H])c(N(c2c([2H])c([2H])c([2H])c3c([2H])c([2H])c([2H])c([2H])c23)c2cccc3oc4c5ccccc5ccc4c23)c([2H])c([2H])c1-c1ccc(-c2ccccc2)cc1 ; [2H]c1c([2H])c(N(c2c([2H])c([2H])c(-c3cccc(-c4cccc5ccccc45)c3)c([2H])c2[2H])c2cccc3oc4c5ccccc5ccc4c23)c([2H])c([2H])c1-c1ccc(-c2ccccc2)cc1]

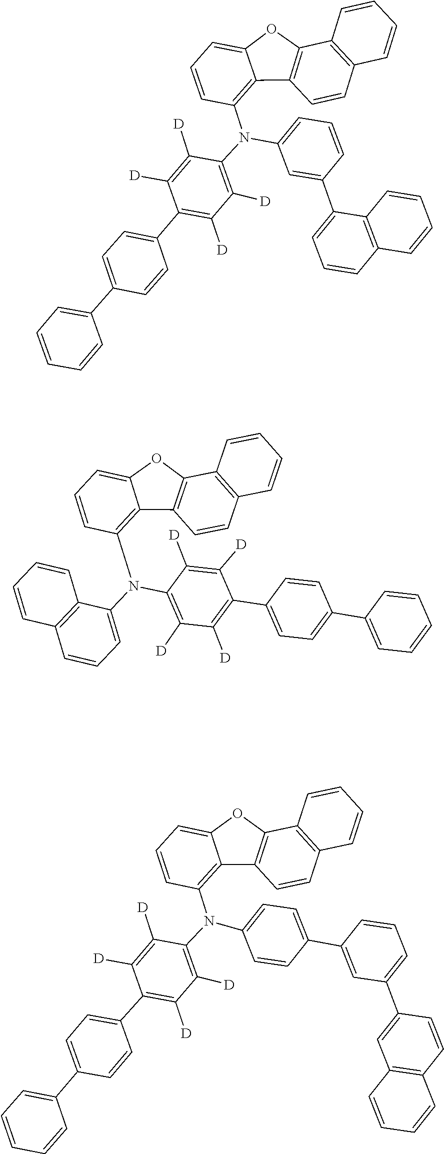 [SMILES: [2H]c1c([2H])c(N(c2cccc(-c3cccc4ccccc34)c2)c2cccc3oc4c5ccccc5ccc4c23)c([2H])c([2H])c1-c1ccc(-c2ccccc2)cc1 ; [2H]c1c([2H])c(N(c2cccc3ccccc23)c2cccc3oc4c5ccccc5ccc4c23)c([2H])c([2H])c1-c1ccc(-c2ccccc2)cc1 ; [2H]c1c([2H])c(N(c2ccc(-c3cccc(-c4ccc5ccccc5c4)c3)cc2)c2cccc3oc4c5ccccc5ccc4c23)c([2H])c([2H])c1-c1ccc(-c2ccccc2)cc1]

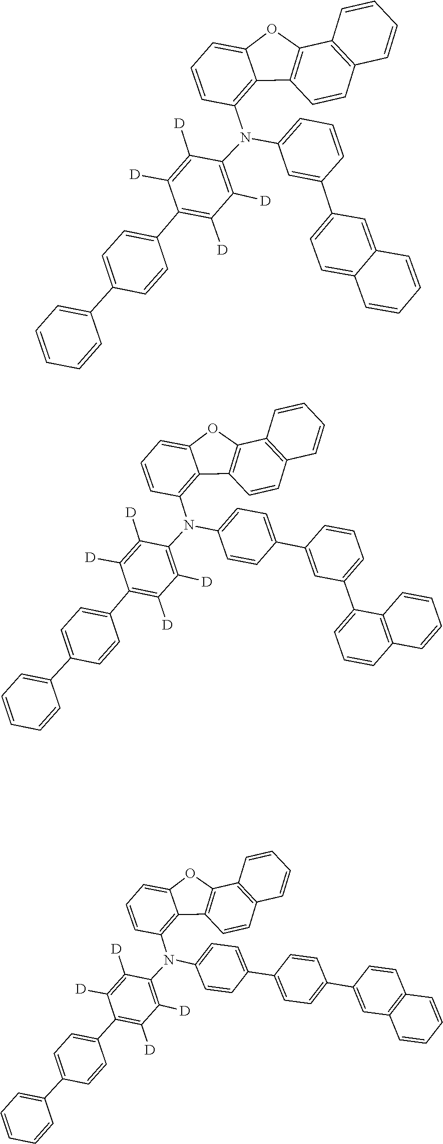 [SMILES: [2H]c1c([2H])c(N(c2cccc(-c3ccc4ccccc4c3)c2)c2cccc3oc4c5ccccc5ccc4c23)c([2H])c([2H])c1-c1ccc(-c2ccccc2)cc1 ; [2H]c1c([2H])c(N(c2ccc(-c3cccc(-c4cccc5ccccc45)c3)cc2)c2cccc3oc4c5ccccc5ccc4c23)c([2H])c([2H])c1-c1ccc(-c2ccccc2)cc1 ; [2H]c1c([2H])c(N(c2ccc(-c3ccc(-c4ccc5ccccc5c4)cc3)cc2)c2cccc3oc4c5ccccc5ccc4c23)c([2H])c([2H])c1-c1ccc(-c2ccccc2)cc1]

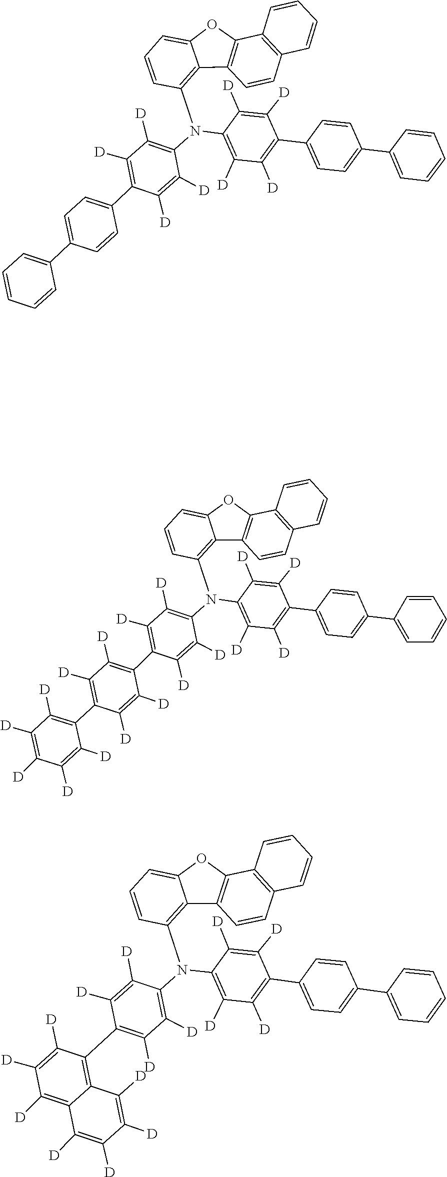 [SMILES: [2H]c1c([2H])c(N(c2c([2H])c([2H])c(-c3ccc(-c4ccccc4)cc3)c([2H])c2[2H])c2cccc3oc4c5ccccc5ccc4c23)c([2H])c([2H])c1-c1ccc(-c2ccccc2)cc1 ; [2H]c1c([2H])c([2H])c(-c2c([2H])c([2H])c(-c3c([2H])c([2H])c(N(c4c([2H])c([2H])c(-c5ccc(-c6ccccc6)cc5)c([2H])c4[2H])c4cccc5oc6c7ccccc7ccc6c45)c([2H])c3[2H])c([2H])c2[2H])c([2H])c1[2H] ; [2H]c1c([2H])c(N(c2c([2H])c([2H])c(-c3c([2H])c([2H])c([2H])c4c([2H])c([2H])c([2H])c([2H])c34)c([2H])c2[2H])c2cccc3oc4c5ccccc5ccc4c23)c([2H])c([2H])c1-c1ccc(-c2ccccc2)cc1]

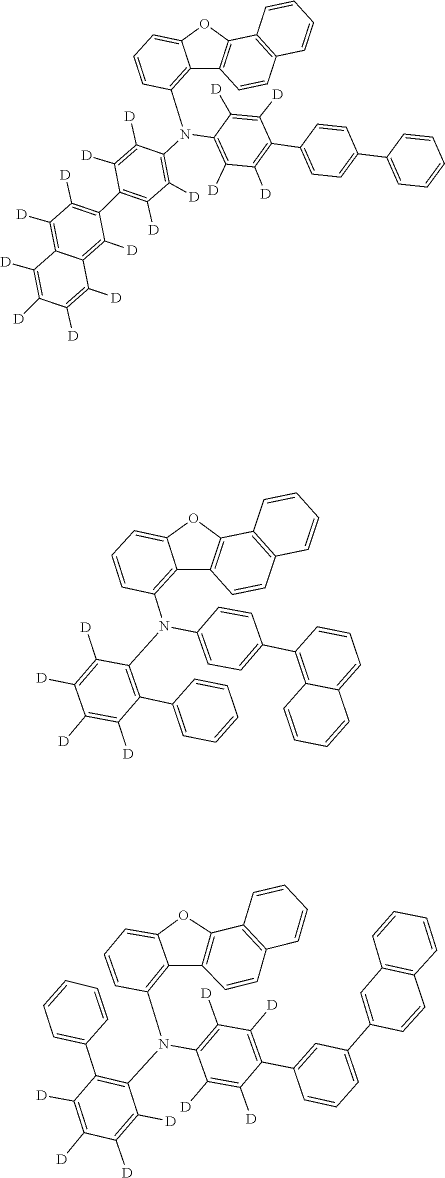 [SMILES: [2H]c1c([2H])c(N(c2c([2H])c([2H])c(-c3c([2H])c([2H])c4c([2H])c([2H])c([2H])c([2H])c4c3[2H])c([2H])c2[2H])c2cccc3oc4c5ccccc5ccc4c23)c([2H])c([2H])c1-c1ccc(-c2ccccc2)cc1 ; [2H]c1c([2H])c([2H])c(N(c2ccc(-c3cccc4ccccc34)cc2)c2cccc3oc4c5ccccc5ccc4c23)c(-c2ccccc2)c1[2H] ; [2H]c1c([2H])c([2H])c(N(c2c([2H])c([2H])c(-c3cccc(-c4ccc5ccccc5c4)c3)c([2H])c2[2H])c2cccc3oc4c5ccccc5ccc4c23)c(-c2ccccc2)c1[2H]]

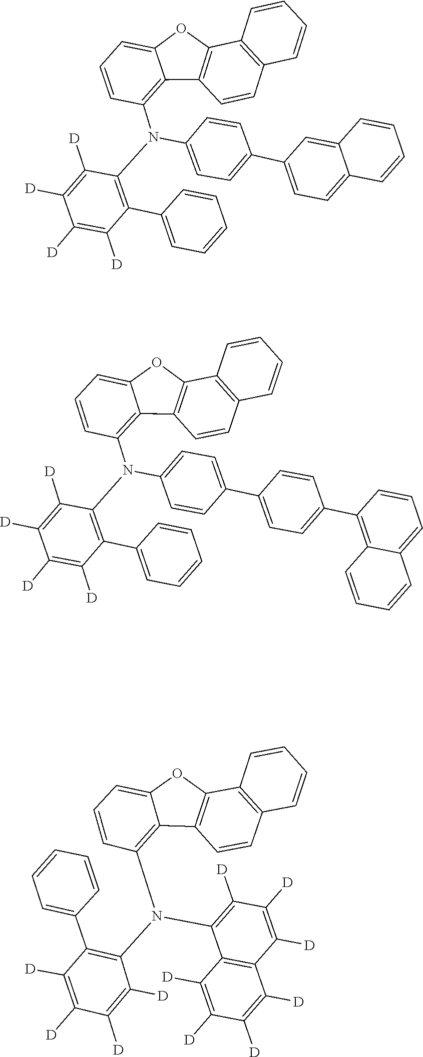 [SMILES: [2H]c1c([2H])c([2H])c(N(c2ccc(-c3ccc4ccccc4c3)cc2)c2cccc3oc4c5ccccc5ccc4c23)c(-c2ccccc2)c1[2H] ; [2H]c1c([2H])c([2H])c(N(c2ccc(-c3ccc(-c4cccc5ccccc45)cc3)cc2)c2cccc3oc4c5ccccc5ccc4c23)c(-c2ccccc2)c1[2H] ; [2H]c1c([2H])c([2H])c(N(c2c([2H])c([2H])c([2H])c3c([2H])c([2H])c([2H])c([2H])c23)c2cccc3oc4c5ccccc5ccc4c23)c(-c2ccccc2)c1[2H]]

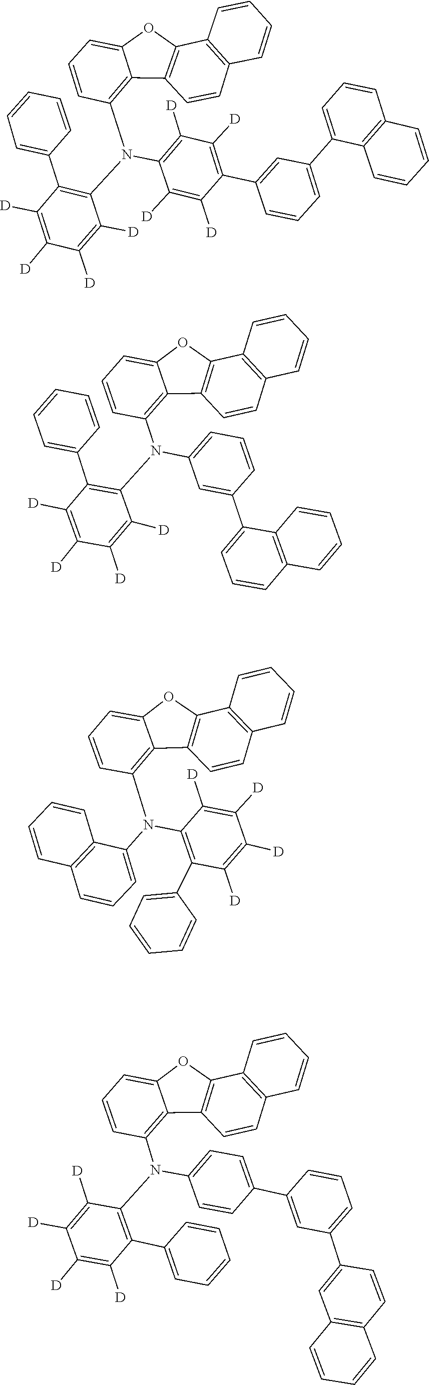 [SMILES: [2H]c1c([2H])c([2H])c(N(c2c([2H])c([2H])c(-c3cccc(-c4cccc5ccccc45)c3)c([2H])c2[2H])c2cccc3oc4c5ccccc5ccc4c23)c(-c2ccccc2)c1[2H] ; [2H]c1c([2H])c([2H])c(N(c2cccc(-c3cccc4ccccc34)c2)c2cccc3oc4c5ccccc5ccc4c23)c(-c2ccccc2)c1[2H] ; [2H]c1c([2H])c([2H])c(N(c2cccc3ccccc23)c2cccc3oc4c5ccccc5ccc4c23)c(-c2ccccc2)c1[2H] ; [2H]c1c([2H])c([2H])c(N(c2ccc(-c3cccc(-c4ccc5ccccc5c4)c3)cc2)c2cccc3oc4c5ccccc5ccc4c23)c(-c2ccccc2)c1[2H]]

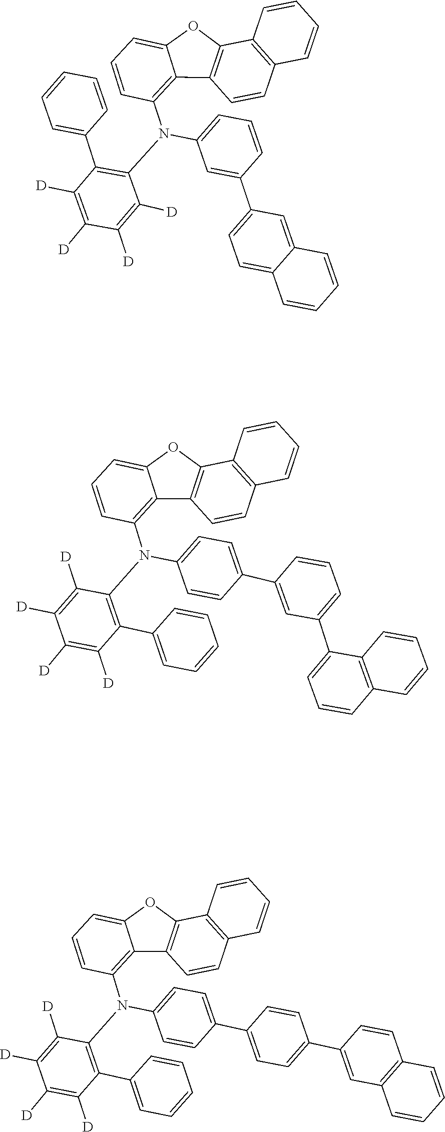 [SMILES: [2H]c1c([2H])c([2H])c(N(c2cccc(-c3ccc4ccccc4c3)c2)c2cccc3oc4c5ccccc5ccc4c23)c(-c2ccccc2)c1[2H] ; [2H]c1c([2H])c([2H])c(N(c2ccc(-c3cccc(-c4cccc5ccccc45)c3)cc2)c2cccc3oc4c5ccccc5ccc4c23)c(-c2ccccc2)c1[2H] ; [2H]c1c([2H])c([2H])c(N(c2ccc(-c3ccc(-c4ccc5ccccc5c4)cc3)cc2)c2cccc3oc4c5ccccc5ccc4c23)c(-c2ccccc2)c1[2H]]

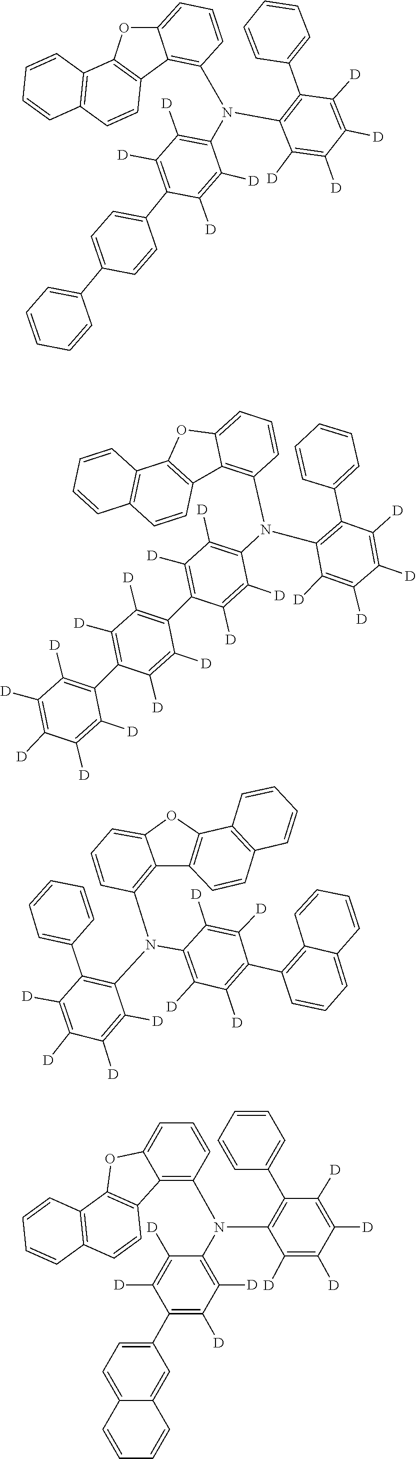

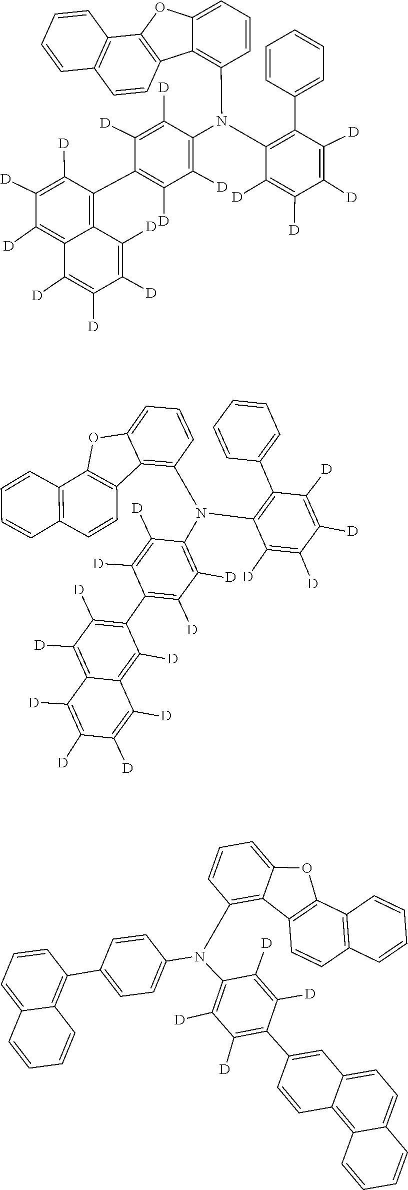 [SMILES: [2H]c1c([2H])c([2H])c(N(c2c([2H])c([2H])c(-c3c([2H])c([2H])c([2H])c4c([2H])c([2H])c([2H])c([2H])c34)c([2H])c2[2H])c2cccc3oc4c5ccccc5ccc4c23)c(-c2ccccc2)c1[2H] ; [2H]c1c([2H])c([2H])c(N(c2c([2H])c([2H])c(-c3c([2H])c([2H])c4c([2H])c([2H])c([2H])c([2H])c4c3[2H])c([2H])c2[2H])c2cccc3oc4c5ccccc5ccc4c23)c(-c2ccccc2)c1[2H] ; [2H]c1c([2H])c(N(c2ccc(-c3cccc4ccccc34)cc2)c2cccc3oc4c5ccccc5ccc4c23)c([2H])c([2H])c1-c1ccc2c(ccc3ccccc32)c1]

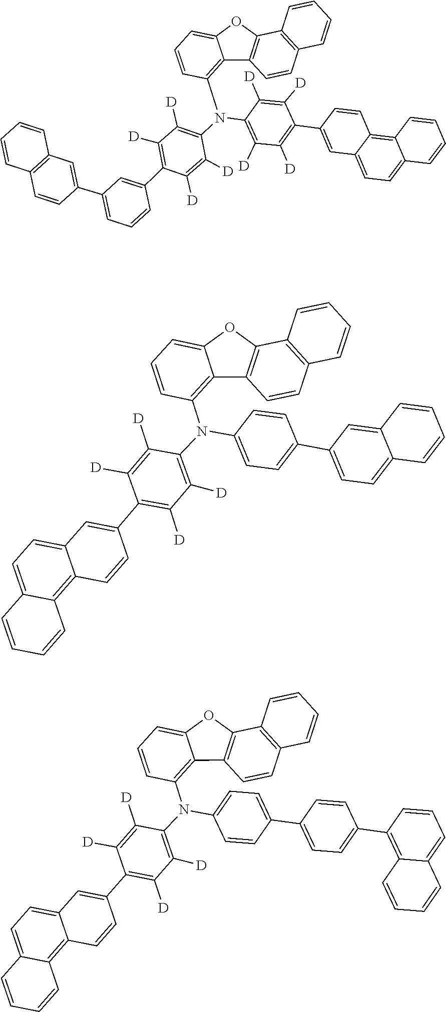 [SMILES: [2H]c1c([2H])c(N(c2c([2H])c([2H])c(-c3ccc4c(ccc5ccccc54)c3)c([2H])c2[2H])c2cccc3oc4c5ccccc5ccc4c23)c([2H])c([2H])c1-c1cccc(-c2ccc3ccccc3c2)c1 ; [2H]c1c([2H])c(N(c2ccc(-c3ccc4ccccc4c3)cc2)c2cccc3oc4c5ccccc5ccc4c23)c([2H])c([2H])c1-c1ccc2c(ccc3ccccc32)c1 ; [2H]c1c([2H])c(N(c2ccc(-c3ccc(-c4cccc5ccccc45)cc3)cc2)c2cccc3oc4c5ccccc5ccc4c23)c([2H])c([2H])c1-c1ccc2c(ccc3ccccc32)c1]

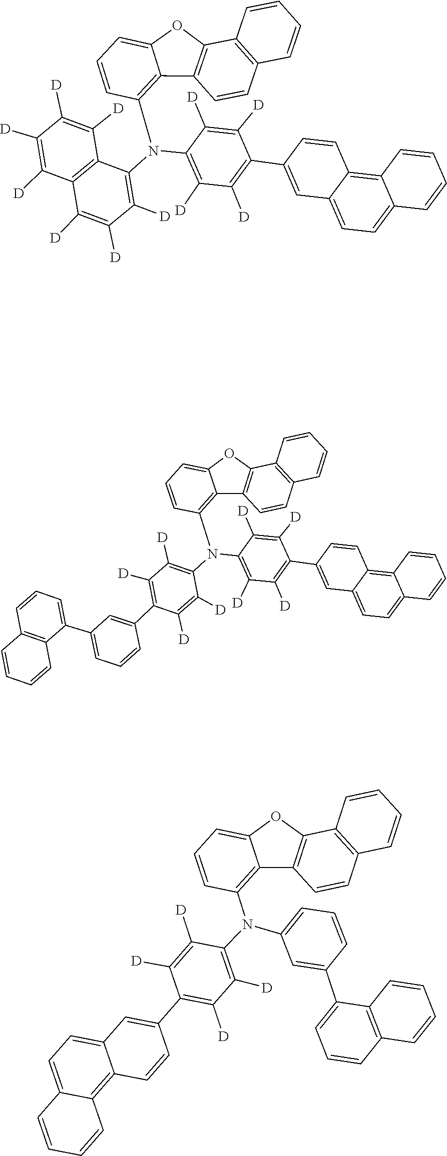 [SMILES: [2H]c1c([2H])c(N(c2c([2H])c([2H])c([2H])c3c([2H])c([2H])c([2H])c([2H])c23)c2cccc3oc4c5ccccc5ccc4c23)c([2H])c([2H])c1-c1ccc2c(ccc3ccccc32)c1 ; [2H]c1c([2H])c(N(c2c([2H])c([2H])c(-c3ccc4c(ccc5ccccc54)c3)c([2H])c2[2H])c2cccc3oc4c5ccccc5ccc4c23)c([2H])c([2H])c1-c1cccc(-c2cccc3ccccc23)c1 ; [2H]c1c([2H])c(N(c2cccc(-c3cccc4ccccc34)c2)c2cccc3oc4c5ccccc5ccc4c23)c([2H])c([2H])c1-c1ccc2c(ccc3ccccc32)c1]

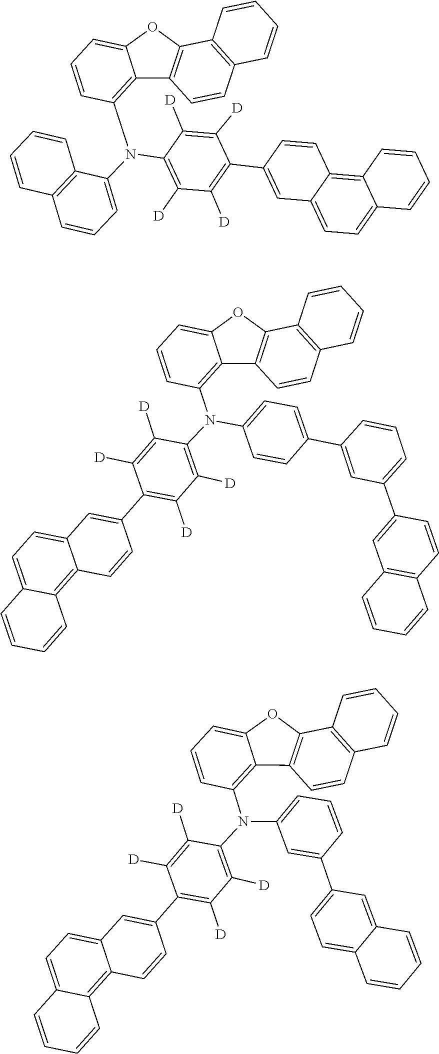 [SMILES: [2H]c1c([2H])c(N(c2cccc3ccccc23)c2cccc3oc4c5ccccc5ccc4c23)c([2H])c([2H])c1-c1ccc2c(ccc3ccccc32)c1 ; [2H]c1c([2H])c(N(c2ccc(-c3cccc(-c4ccc5ccccc5c4)c3)cc2)c2cccc3oc4c5ccccc5ccc4c23)c([2H])c([2H])c1-c1ccc2c(ccc3ccccc32)c1 ; [2H]c1c([2H])c(N(c2cccc(-c3ccc4ccccc4c3)c2)c2cccc3oc4c5ccccc5ccc4c23)c([2H])c([2H])c1-c1ccc2c(ccc3ccccc32)c1]

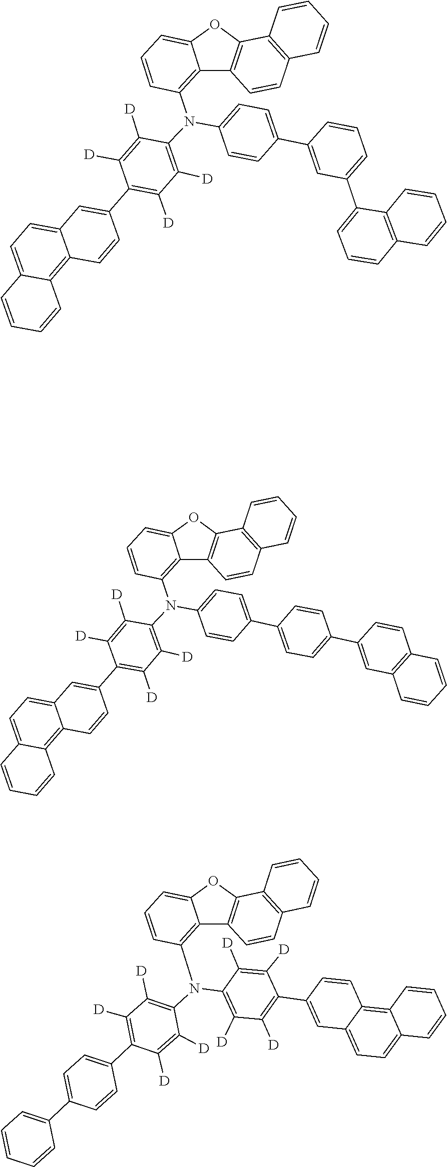 [SMILES: [2H]c1c([2H])c(N(c2ccc(-c3cccc(-c4cccc5ccccc45)c3)cc2)c2cccc3oc4c5ccccc5ccc4c23)c([2H])c([2H])c1-c1ccc2c(ccc3ccccc32)c1 ; [2H]c1c([2H])c(N(c2ccc(-c3ccc(-c4ccc5ccccc5c4)cc3)cc2)c2cccc3oc4c5ccccc5ccc4c23)c([2H])c([2H])c1-c1ccc2c(ccc3ccccc32)c1 ; [2H]c1c([2H])c(N(c2c([2H])c([2H])c(-c3ccc4c(ccc5ccccc54)c3)c([2H])c2[2H])c2cccc3oc4c5ccccc5ccc4c23)c([2H])c([2H])c1-c1ccc(-c2ccccc2)cc1]

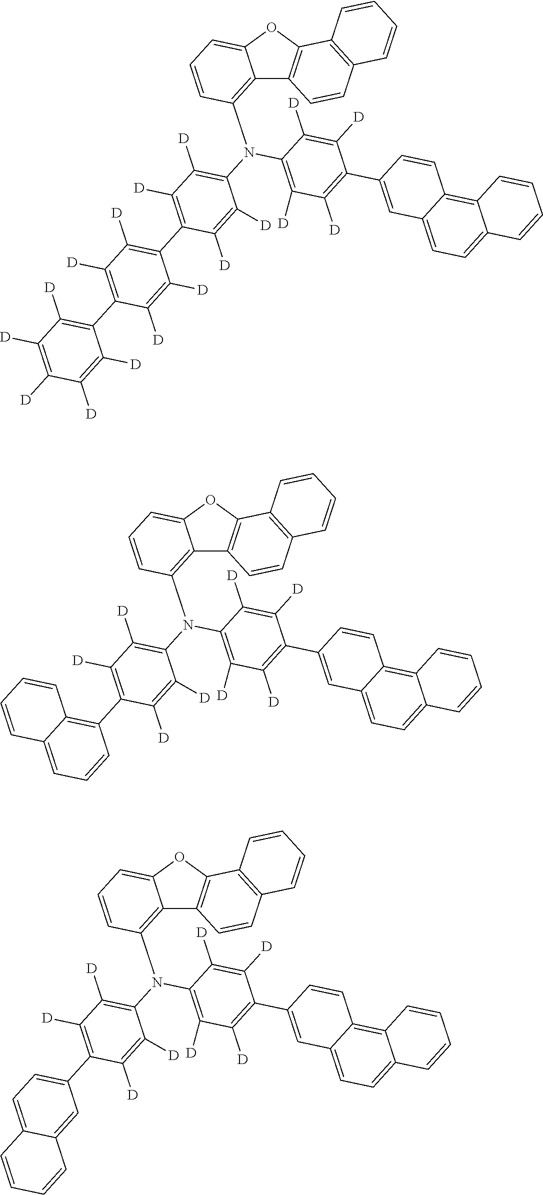 [SMILES: [2H]c1c([2H])c([2H])c(-c2c([2H])c([2H])c(-c3c([2H])c([2H])c(N(c4c([2H])c([2H])c(-c5ccc6c(ccc7ccccc76)c5)c([2H])c4[2H])c4cccc5oc6c7ccccc7ccc6c45)c([2H])c3[2H])c([2H])c2[2H])c([2H])c1[2H] ; [2H]c1c([2H])c(N(c2c([2H])c([2H])c(-c3cccc4ccccc34)c([2H])c2[2H])c2cccc3oc4c5ccccc5ccc4c23)c([2H])c([2H])c1-c1ccc2c(ccc3ccccc32)c1 ; [2H]c1c([2H])c(N(c2c([2H])c([2H])c(-c3ccc4c(ccc5ccccc54)c3)c([2H])c2[2H])c2cccc3oc4c5ccccc5ccc4c23)c([2H])c([2H])c1-c1ccc2ccccc2c1]

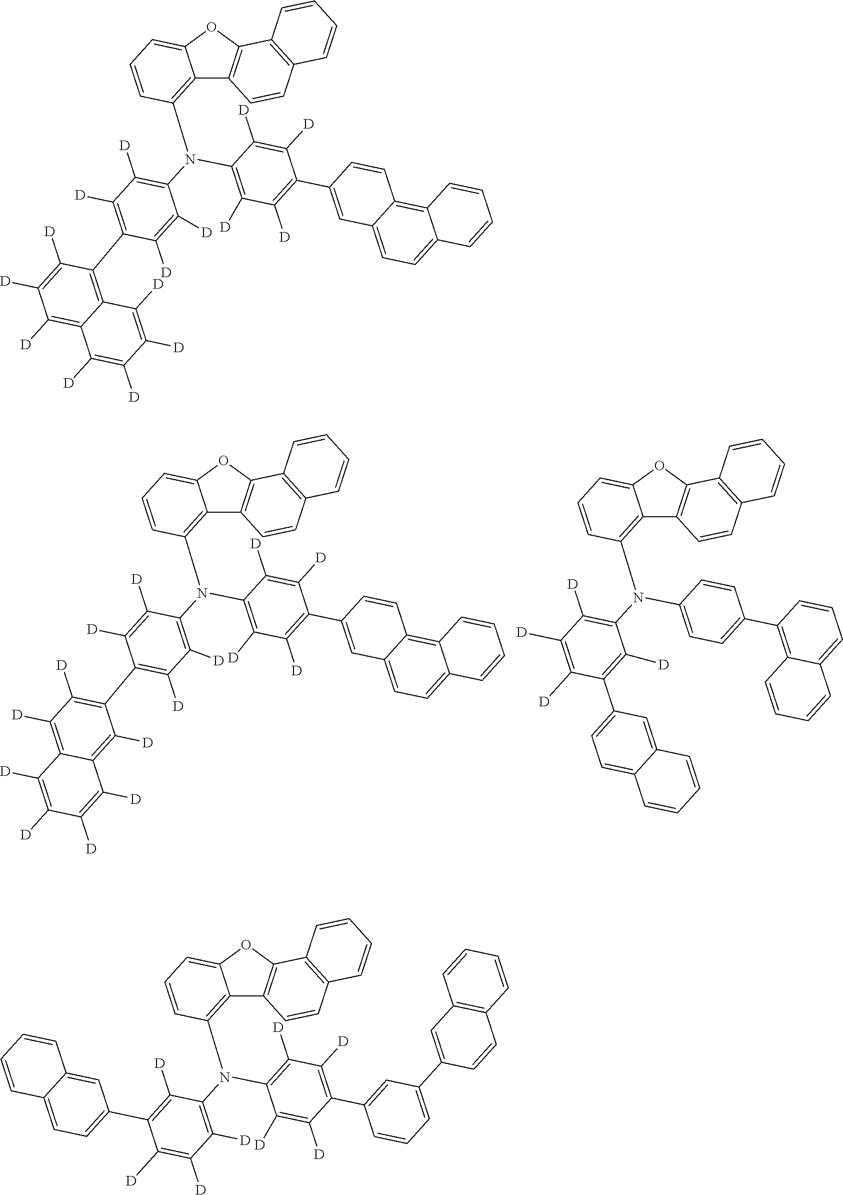

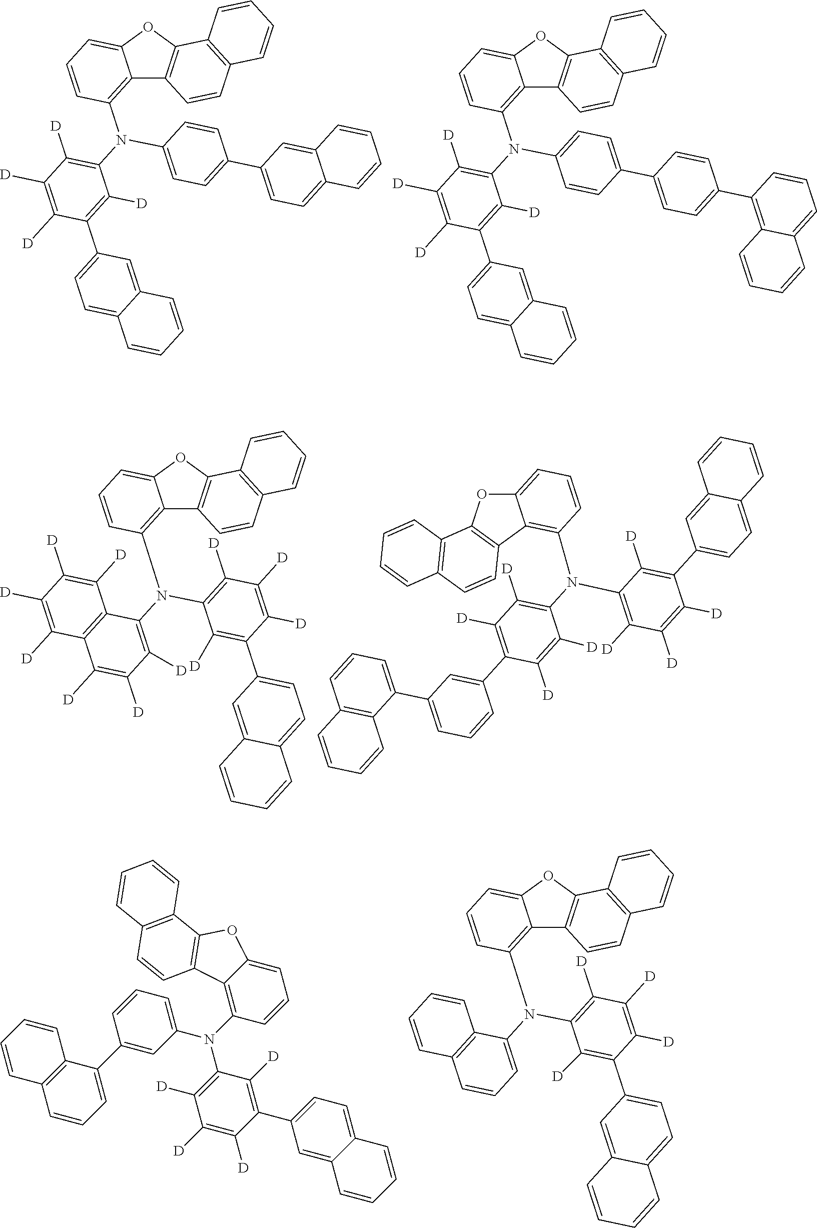

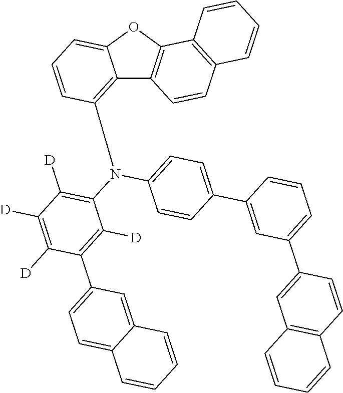 [SMILES: [2H]c1c([2H])c(-c2ccc3ccccc3c2)c([2H])c(N(c2ccc(-c3cccc(-c4ccc5ccccc5c4)c3)cc2)c2cccc3oc4c5ccccc5ccc4c23)c1[2H]]

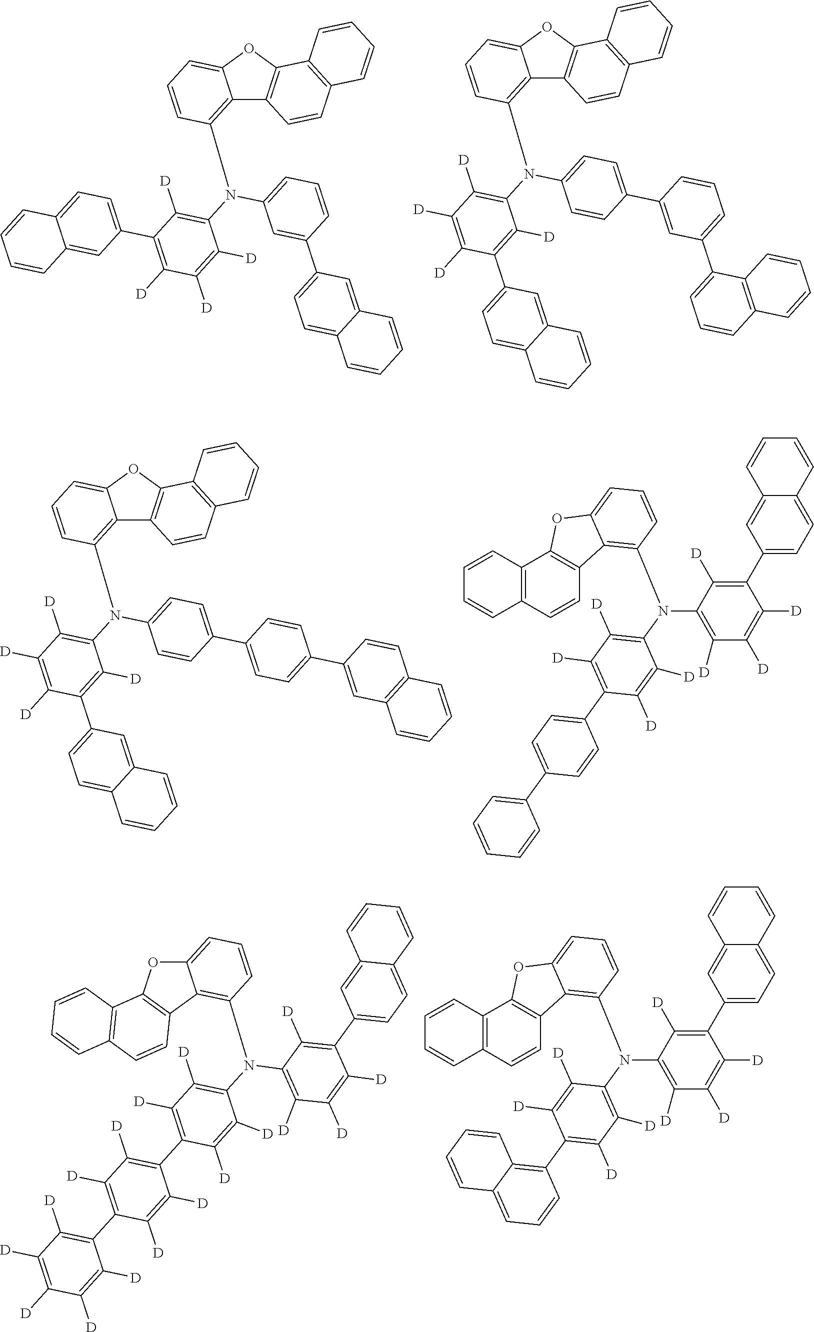

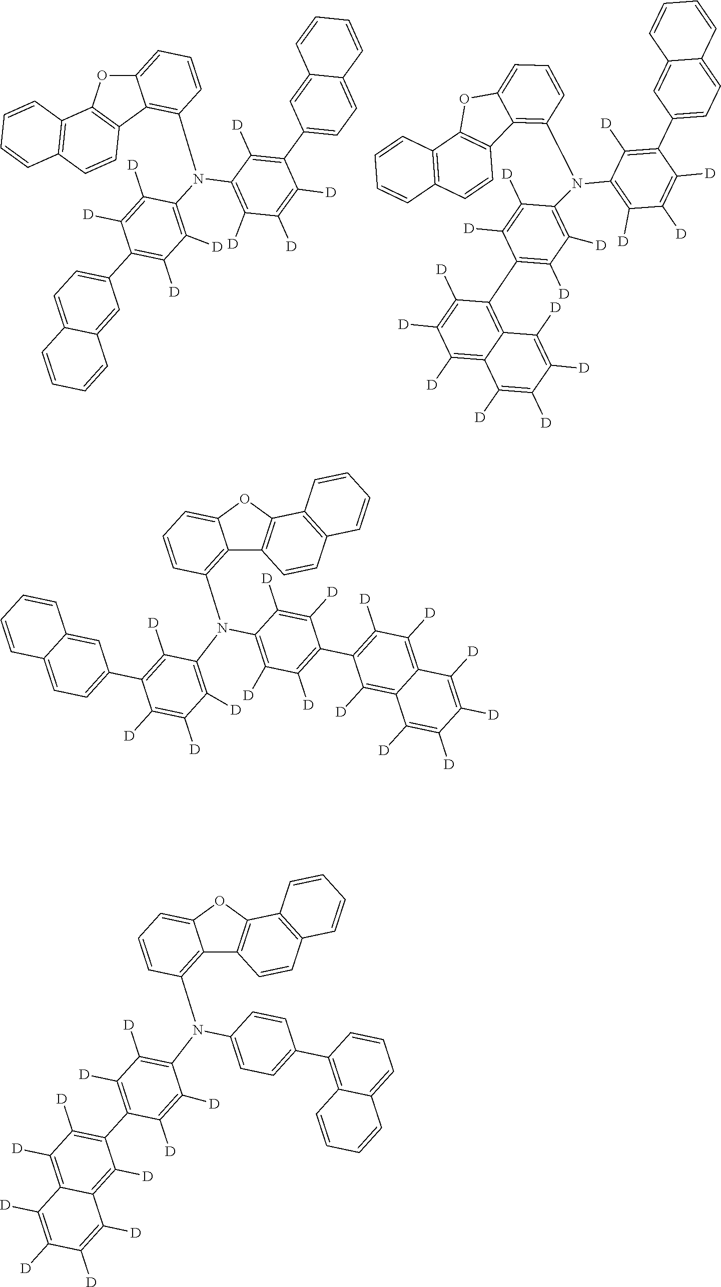

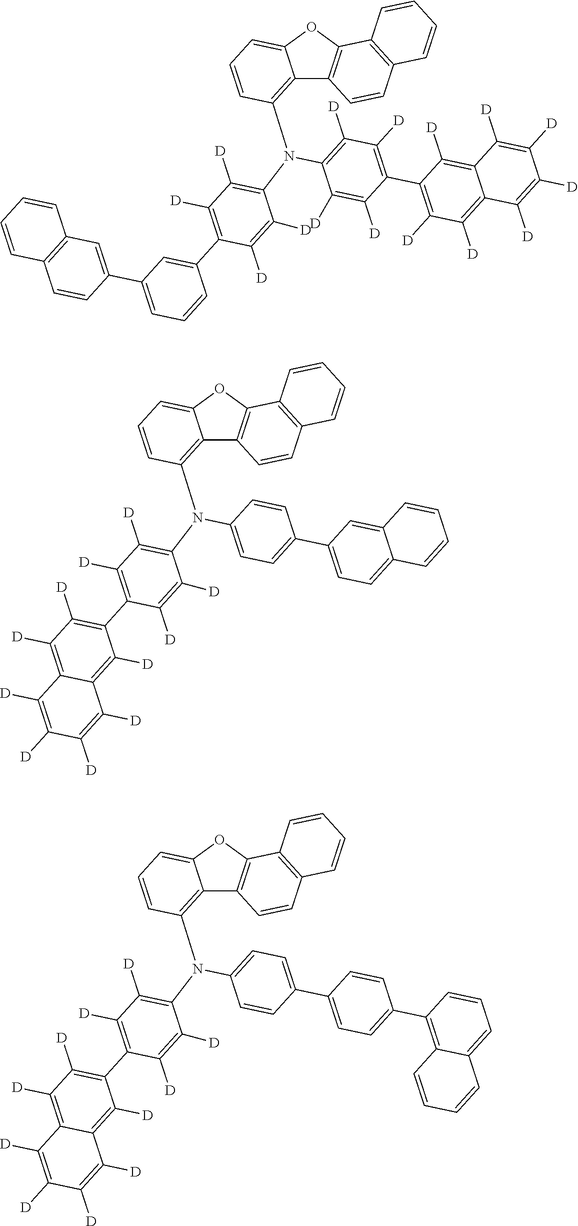 [SMILES: [2H]c1c([2H])c(N(c2c([2H])c([2H])c(-c3c([2H])c([2H])c4c([2H])c([2H])c([2H])c([2H])c4c3[2H])c([2H])c2[2H])c2cccc3oc4c5ccccc5ccc4c23)c([2H])c([2H])c1-c1cccc(-c2ccc3ccccc3c2)c1 ; [2H]c1c([2H])c(N(c2ccc(-c3ccc4ccccc4c3)cc2)c2cccc3oc4c5ccccc5ccc4c23)c([2H])c([2H])c1-c1c([2H])c([2H])c2c([2H])c([2H])c([2H])c([2H])c2c1[2H] ; [2H]c1c([2H])c(N(c2ccc(-c3ccc(-c4cccc5ccccc45)cc3)cc2)c2cccc3oc4c5ccccc5ccc4c23)c([2H])c([2H])c1-c1c([2H])c([2H])c2c([2H])c([2H])c([2H])c([2H])c2c1[2H]]

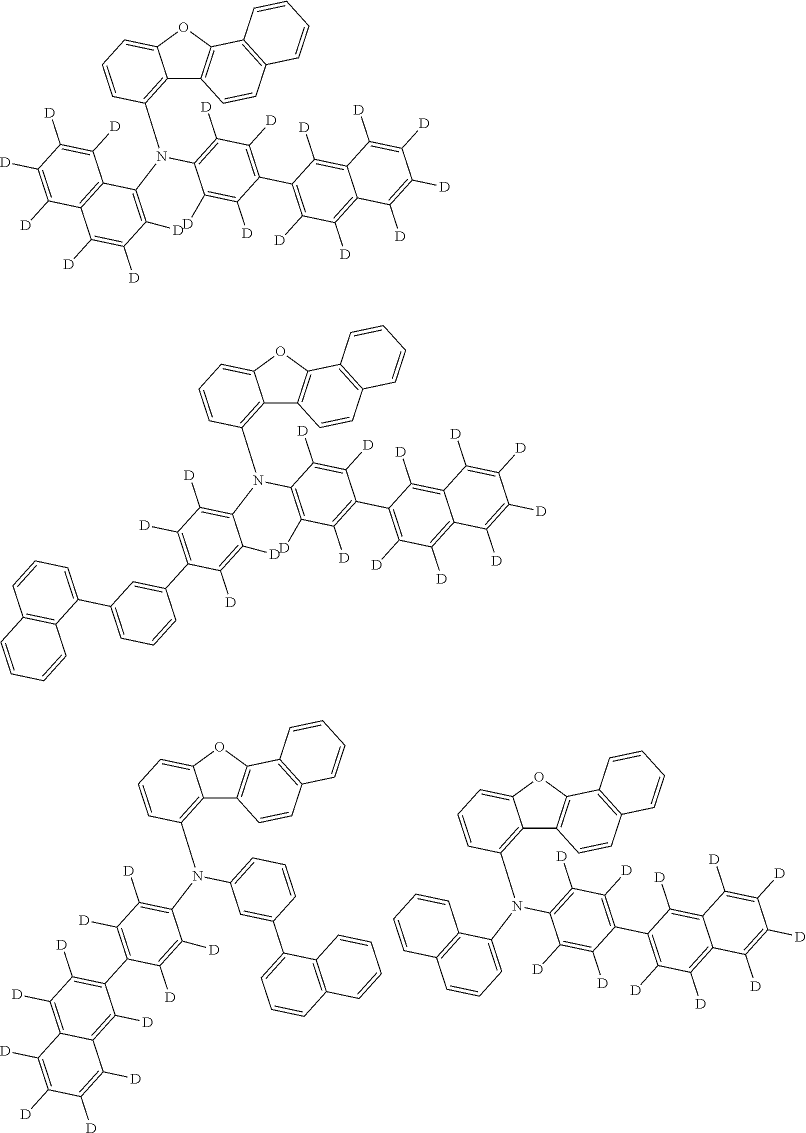

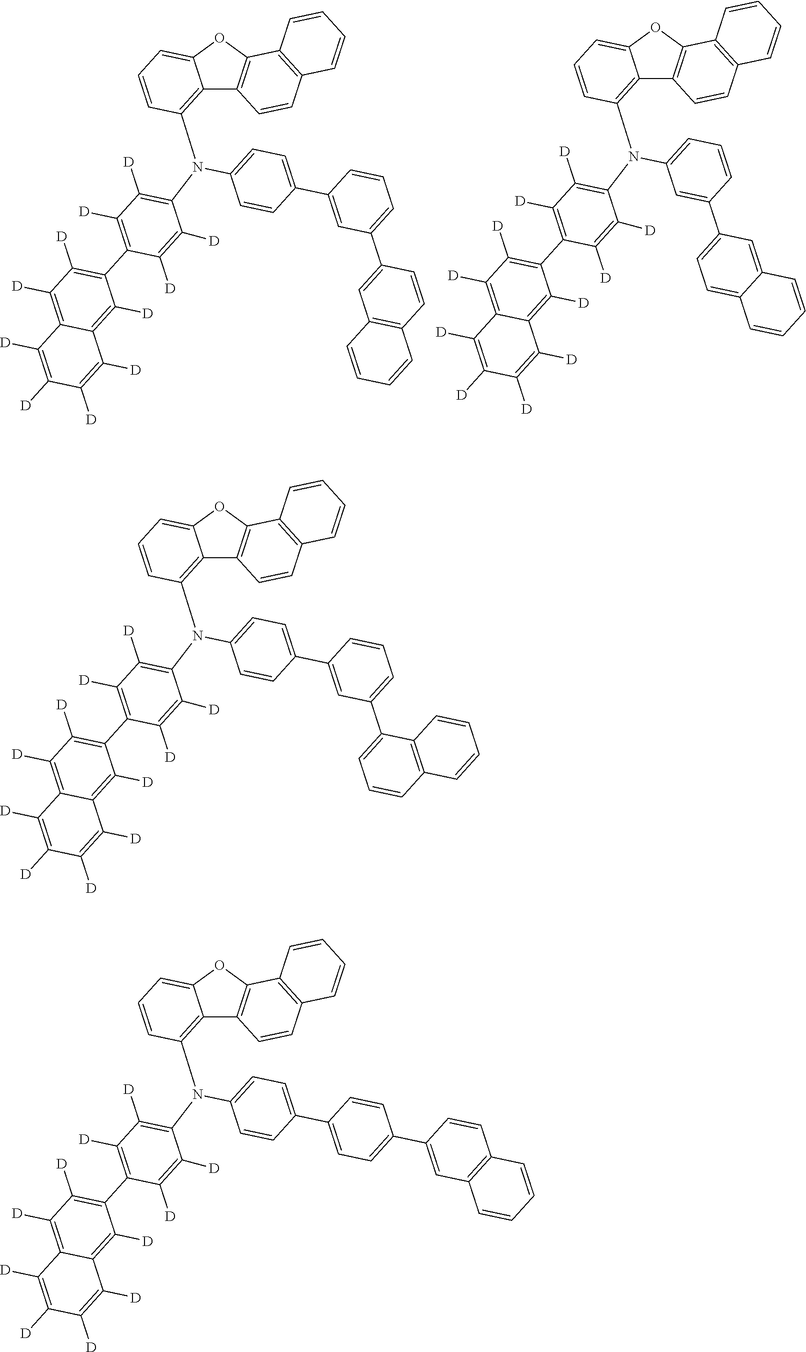

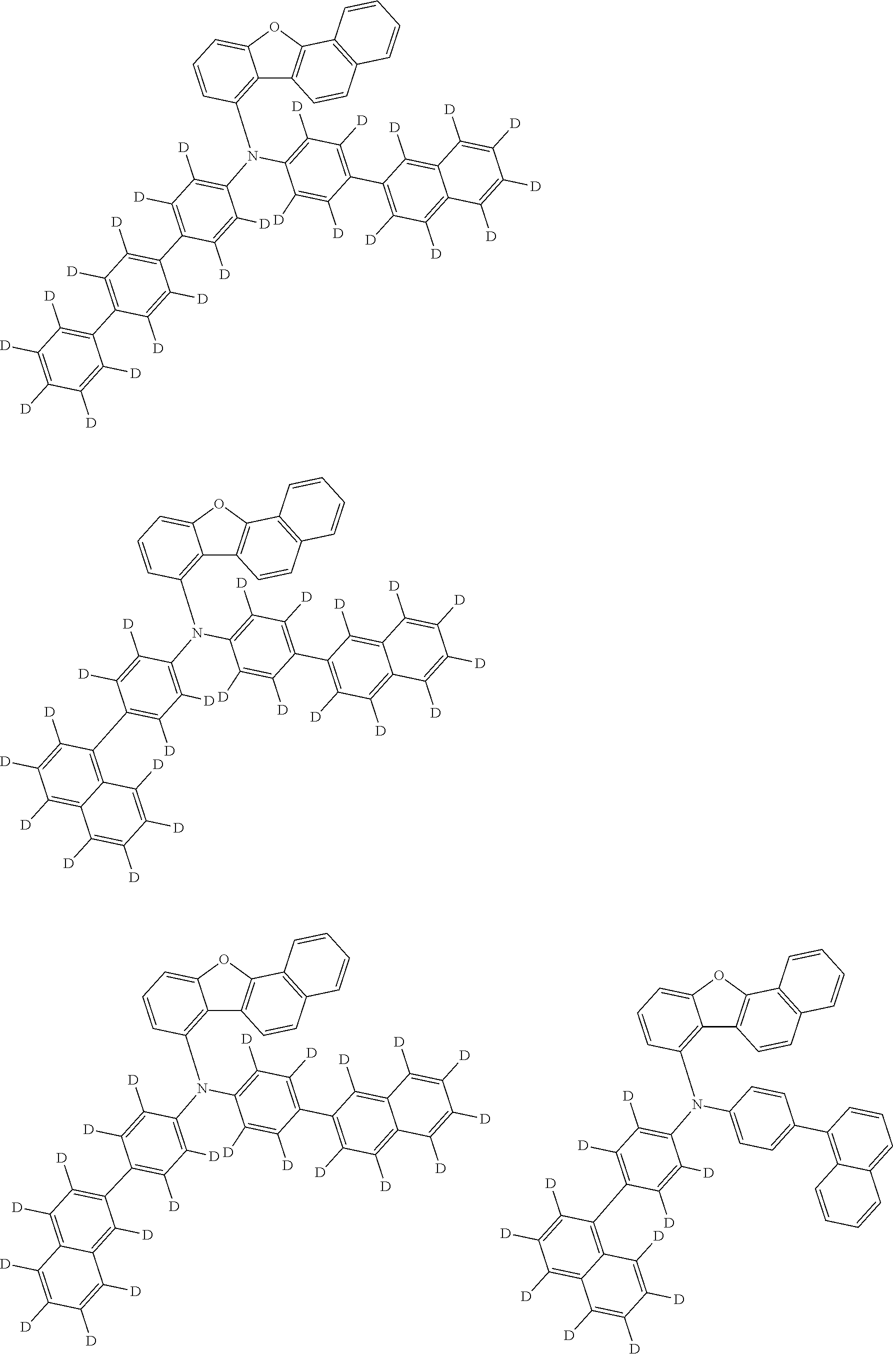

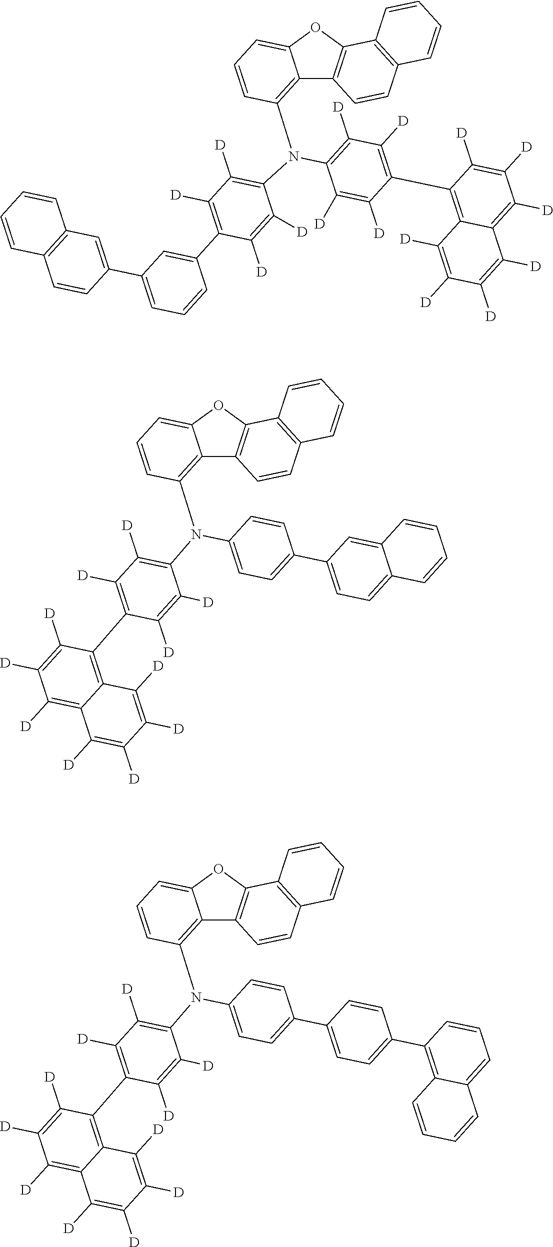 [SMILES: [2H]c1c([2H])c(N(c2c([2H])c([2H])c(-c3c([2H])c([2H])c([2H])c4c([2H])c([2H])c([2H])c([2H])c34)c([2H])c2[2H])c2cccc3oc4c5ccccc5ccc4c23)c([2H])c([2H])c1-c1cccc(-c2ccc3ccccc3c2)c1 ; [2H]c1c([2H])c(N(c2ccc(-c3ccc4ccccc4c3)cc2)c2cccc3oc4c5ccccc5ccc4c23)c([2H])c([2H])c1-c1c([2H])c([2H])c([2H])c2c([2H])c([2H])c([2H])c([2H])c12 ; [2H]c1c([2H])c(N(c2ccc(-c3ccc(-c4cccc5ccccc45)cc3)cc2)c2cccc3oc4c5ccccc5ccc4c23)c([2H])c([2H])c1-c1c([2H])c([2H])c([2H])c2c([2H])c([2H])c([2H])c([2H])c12]

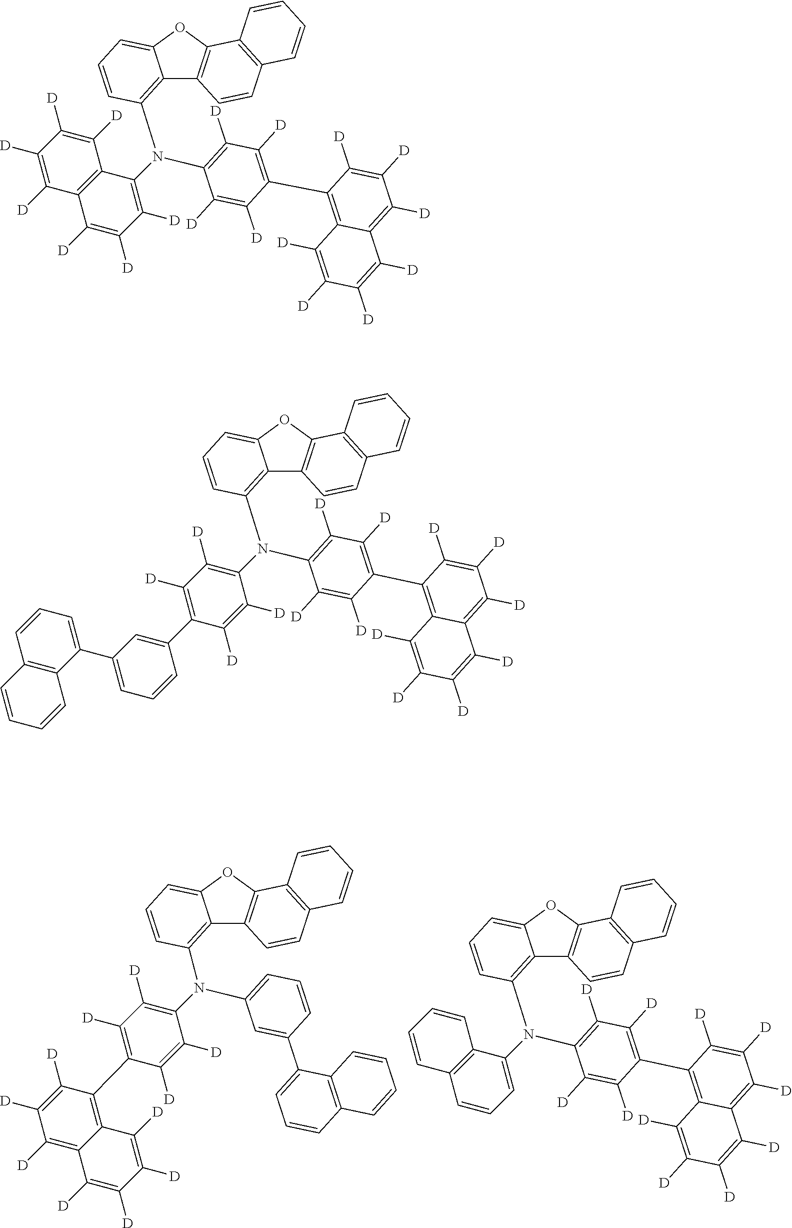

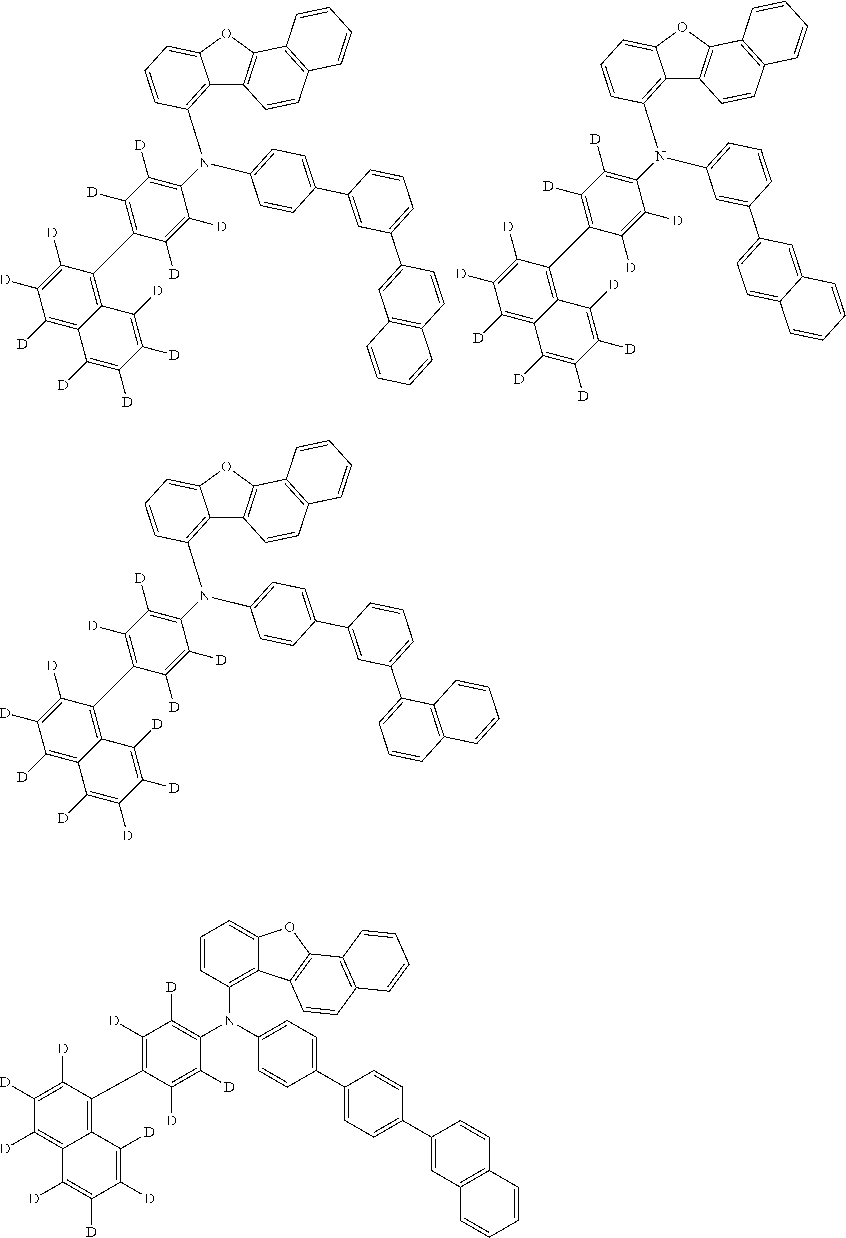

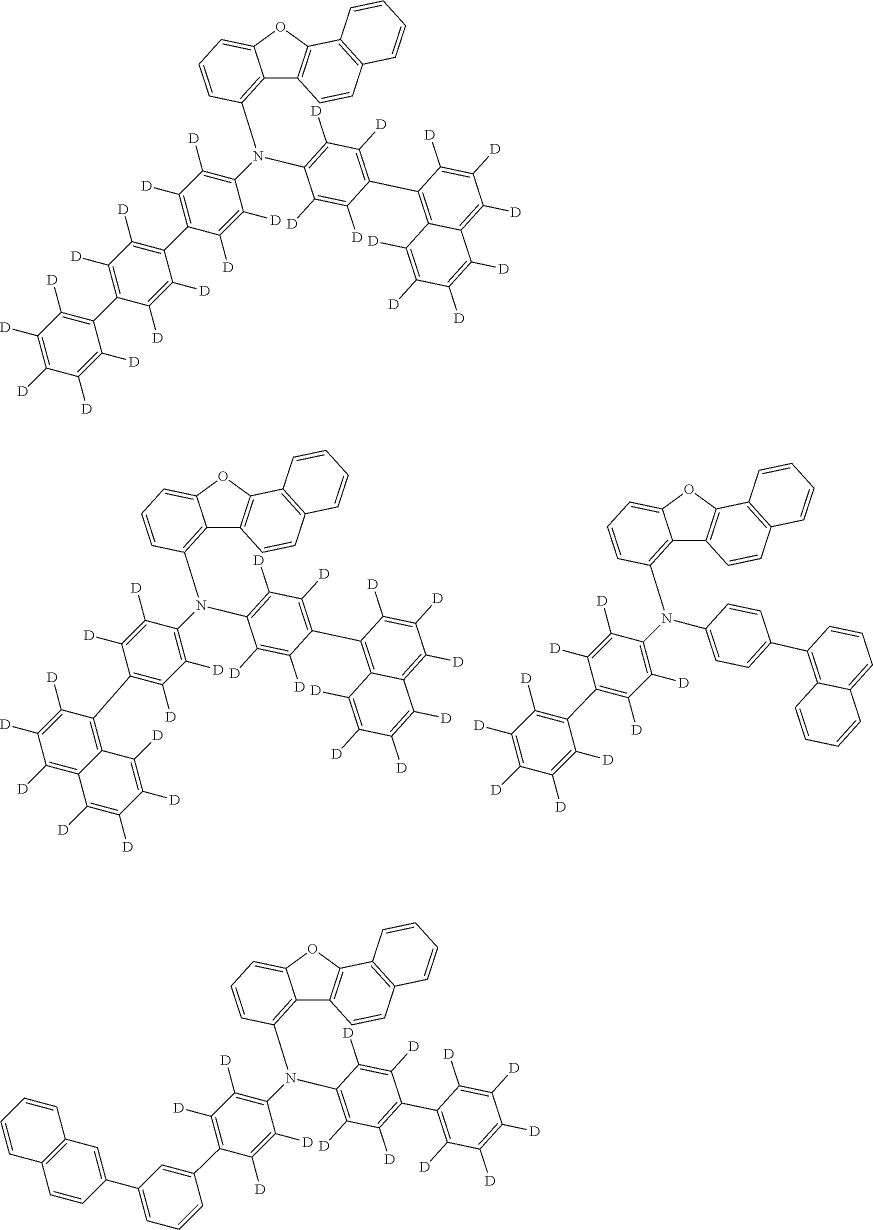

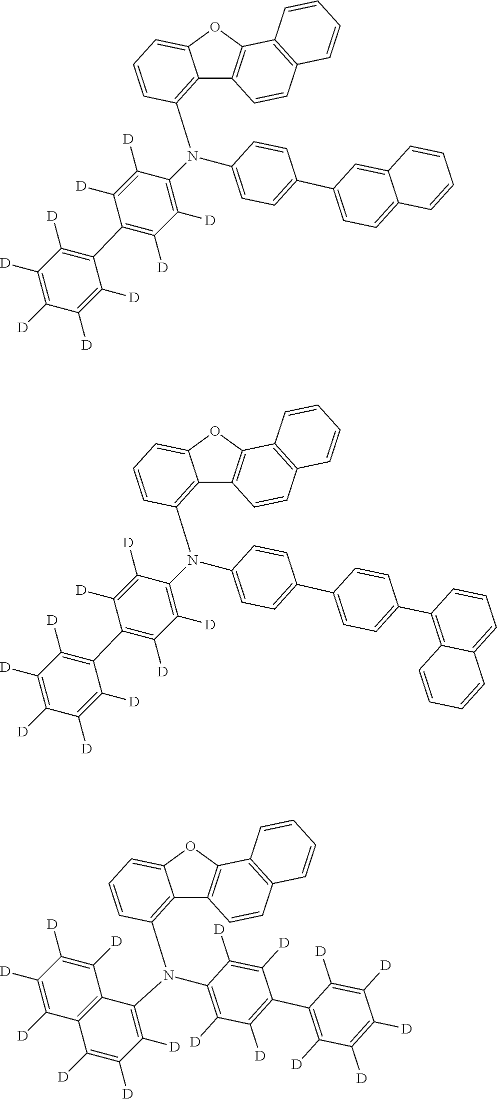 [SMILES: [2H]c1c([2H])c([2H])c(-c2c([2H])c([2H])c(N(c3ccc(-c4ccc5ccccc5c4)cc3)c3cccc4oc5c6ccccc6ccc5c34)c([2H])c2[2H])c([2H])c1[2H] ; [2H]c1c([2H])c([2H])c(-c2c([2H])c([2H])c(N(c3ccc(-c4ccc(-c5cccc6ccccc56)cc4)cc3)c3cccc4oc5c6ccccc6ccc5c34)c([2H])c2[2H])c([2H])c1[2H] ; [2H]c1c([2H])c([2H])c(-c2c([2H])c([2H])c(N(c3c([2H])c([2H])c([2H])c4c([2H])c([2H])c([2H])c([2H])c34)c3cccc4oc5c6ccccc6ccc5c34)c([2H])c2[2H])c([2H])c1[2H]]

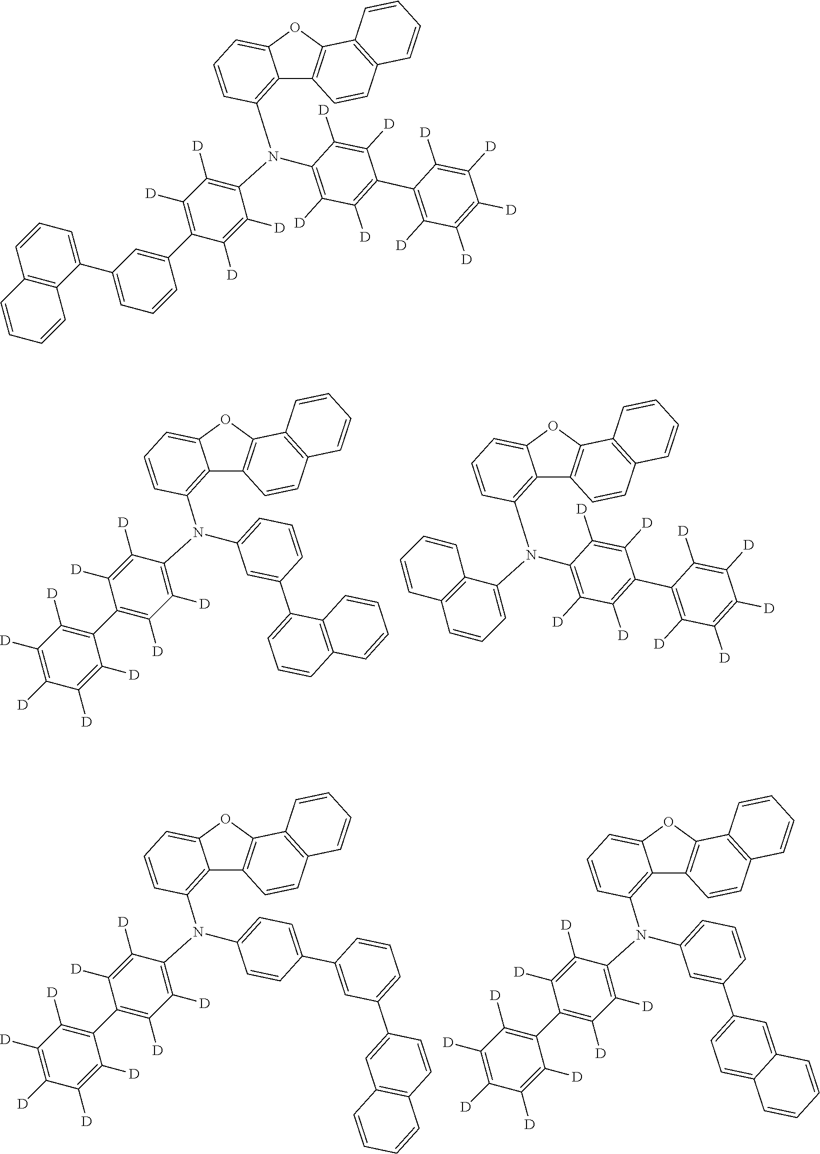

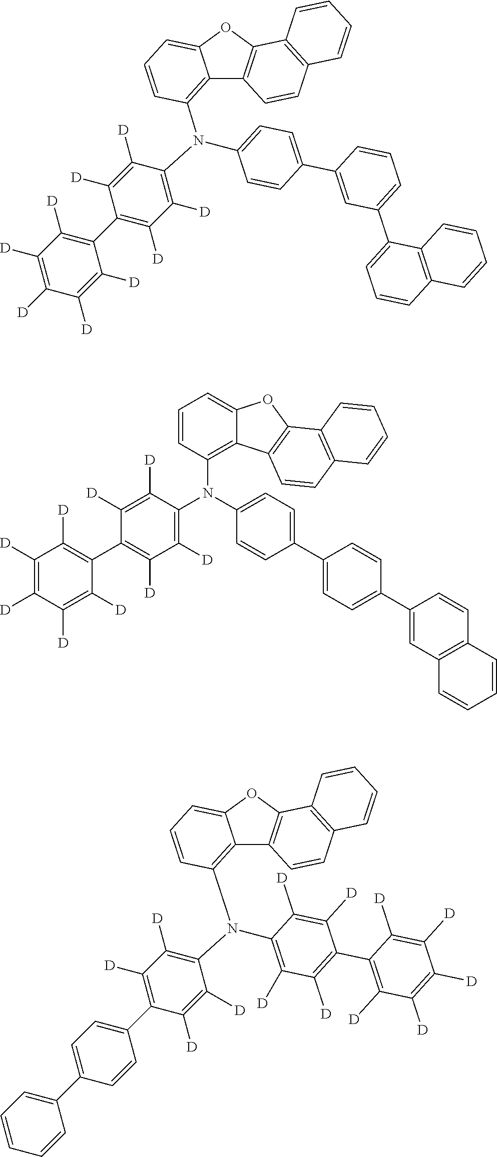 [SMILES: [2H]c1c([2H])c([2H])c(-c2c([2H])c([2H])c(N(c3ccc(-c4cccc(-c5cccc6ccccc56)c4)cc3)c3cccc4oc5c6ccccc6ccc5c34)c([2H])c2[2H])c([2H])c1[2H] ; [2H]c1c([2H])c([2H])c(-c2c([2H])c([2H])c(N(c3ccc(-c4ccc(-c5ccc6ccccc6c5)cc4)cc3)c3cccc4oc5c6ccccc6ccc5c34)c([2H])c2[2H])c([2H])c1[2H] ; [2H]c1c([2H])c([2H])c(-c2c([2H])c([2H])c(N(c3c([2H])c([2H])c(-c4ccc(-c5ccccc5)cc4)c([2H])c3[2H])c3cccc4oc5c6ccccc6ccc5c34)c([2H])c2[2H])c([2H])c1[2H]]

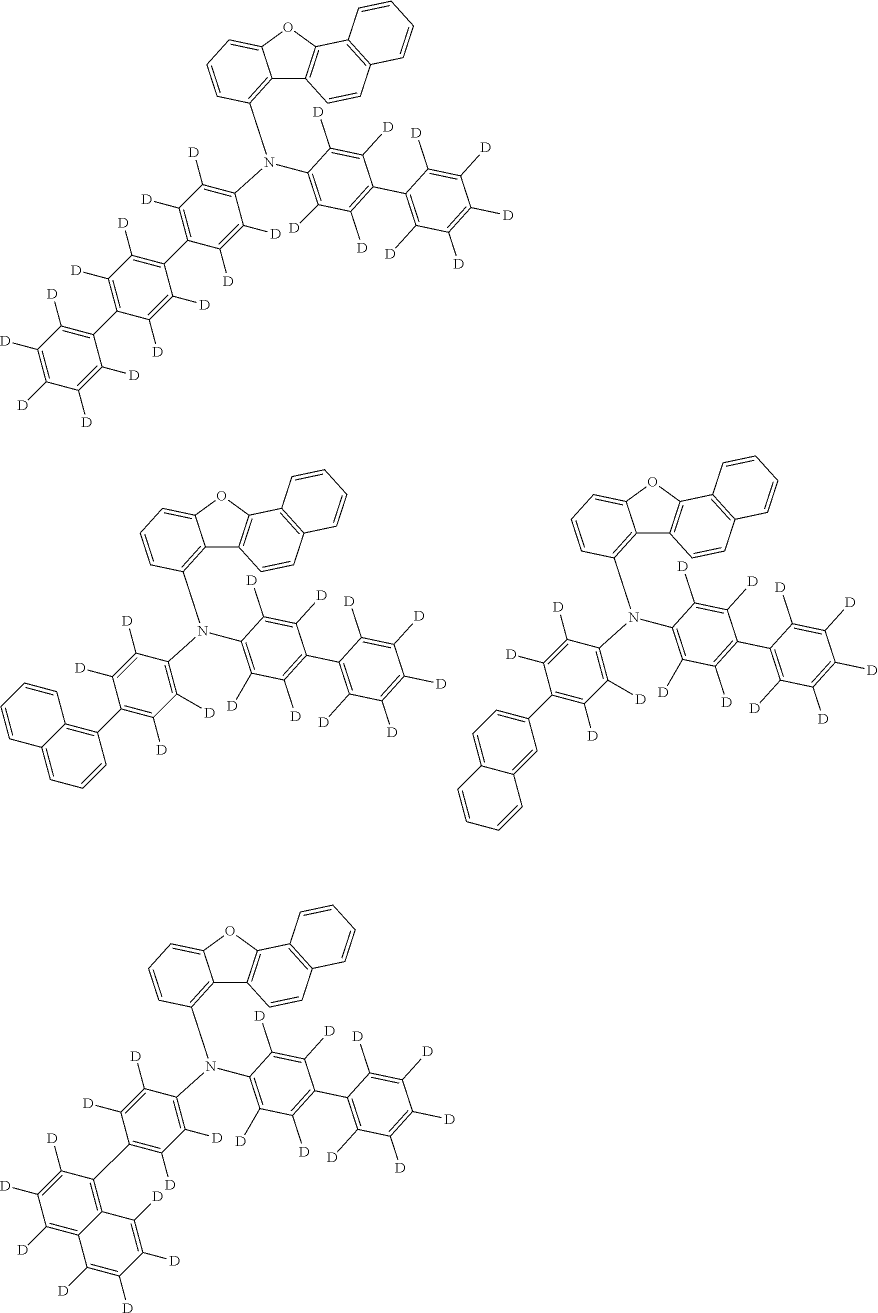

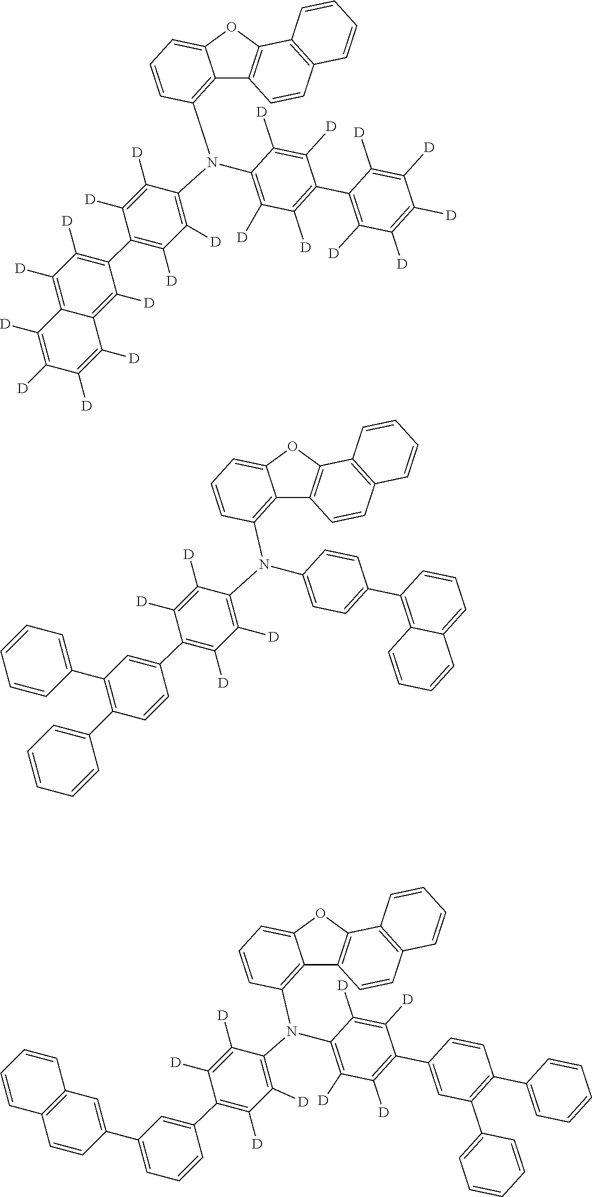 [SMILES: [2H]c1c([2H])c([2H])c(-c2c([2H])c([2H])c(N(c3c([2H])c([2H])c(-c4c([2H])c([2H])c5c([2H])c([2H])c([2H])c([2H])c5c4[2H])c([2H])c3[2H])c3cccc4oc5c6ccccc6ccc5c34)c([2H])c2[2H])c([2H])c1[2H] ; [2H]c1c([2H])c(N(c2ccc(-c3cccc4ccccc34)cc2)c2cccc3oc4c5ccccc5ccc4c23)c([2H])c([2H])c1-c1ccc(-c2ccccc2)c(-c2ccccc2)c1 ; [2H]c1c([2H])c(N(c2c([2H])c([2H])c(-c3ccc(-c4ccccc4)c(-c4ccccc4)c3)c([2H])c2[2H])c2cccc3oc4c5ccccc5ccc4c23)c([2H])c([2H])c1-c1cccc(-c2ccc3ccccc3c2)c1]

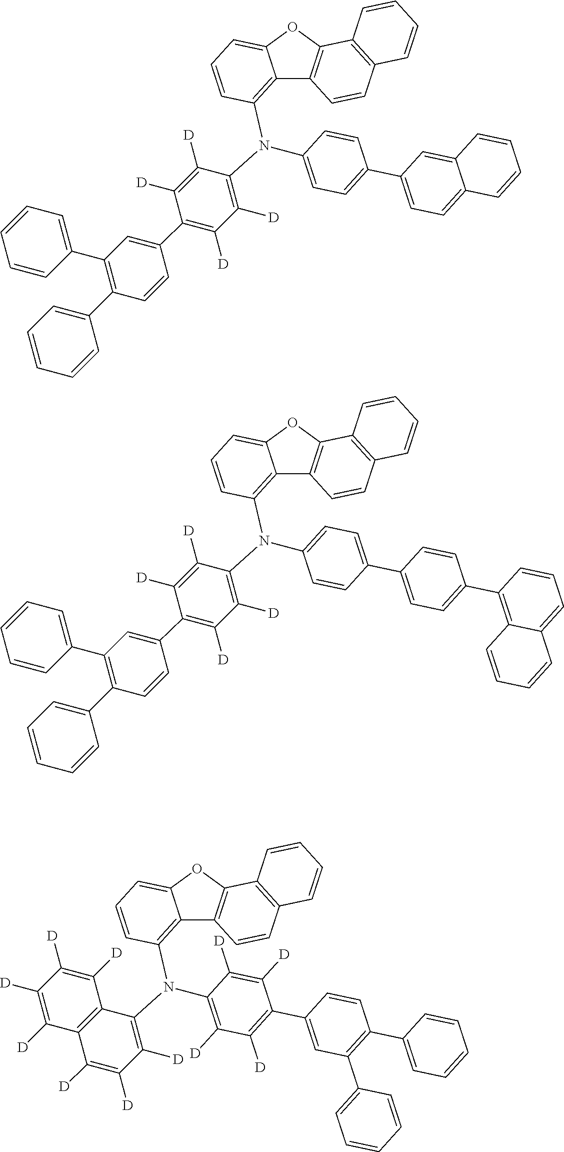 [SMILES: [2H]c1c([2H])c(N(c2ccc(-c3ccc4ccccc4c3)cc2)c2cccc3oc4c5ccccc5ccc4c23)c([2H])c([2H])c1-c1ccc(-c2ccccc2)c(-c2ccccc2)c1 ; [2H]c1c([2H])c(N(c2ccc(-c3ccc(-c4cccc5ccccc45)cc3)cc2)c2cccc3oc4c5ccccc5ccc4c23)c([2H])c([2H])c1-c1ccc(-c2ccccc2)c(-c2ccccc2)c1 ; [2H]c1c([2H])c(N(c2c([2H])c([2H])c([2H])c3c([2H])c([2H])c([2H])c([2H])c23)c2cccc3oc4c5ccccc5ccc4c23)c([2H])c([2H])c1-c1ccc(-c2ccccc2)c(-c2ccccc2)c1]

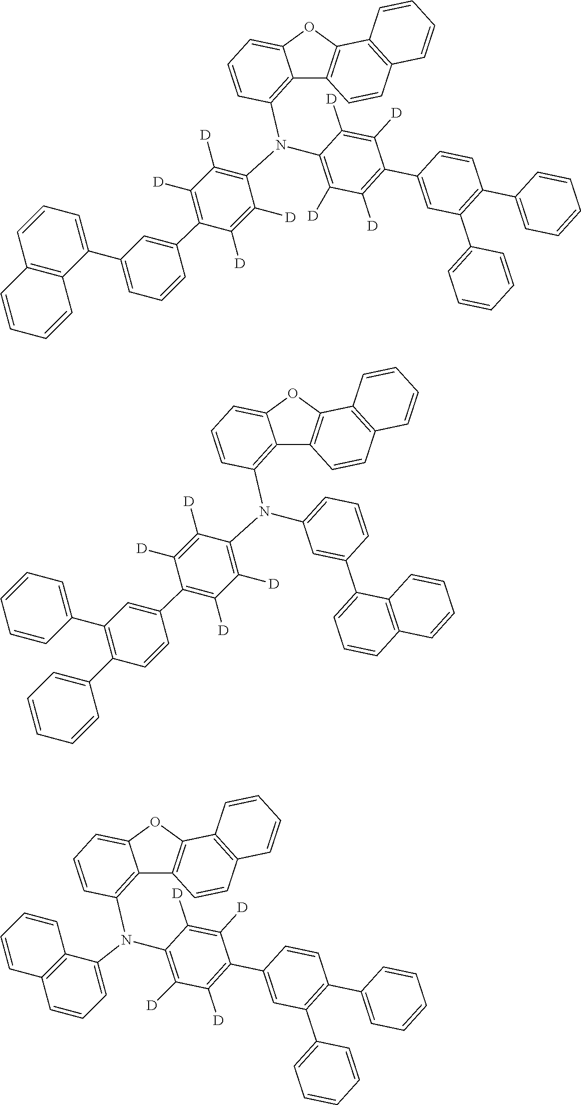 [SMILES: [2H]c1c([2H])c(N(c2c([2H])c([2H])c(-c3ccc(-c4ccccc4)c(-c4ccccc4)c3)c([2H])c2[2H])c2cccc3oc4c5ccccc5ccc4c23)c([2H])c([2H])c1-c1cccc(-c2cccc3ccccc23)c1 ; [2H]c1c([2H])c(N(c2cccc(-c3cccc4ccccc34)c2)c2cccc3oc4c5ccccc5ccc4c23)c([2H])c([2H])c1-c1ccc(-c2ccccc2)c(-c2ccccc2)c1 ; [2H]c1c([2H])c(N(c2cccc3ccccc23)c2cccc3oc4c5ccccc5ccc4c23)c([2H])c([2H])c1-c1ccc(-c2ccccc2)c(-c2ccccc2)c1]

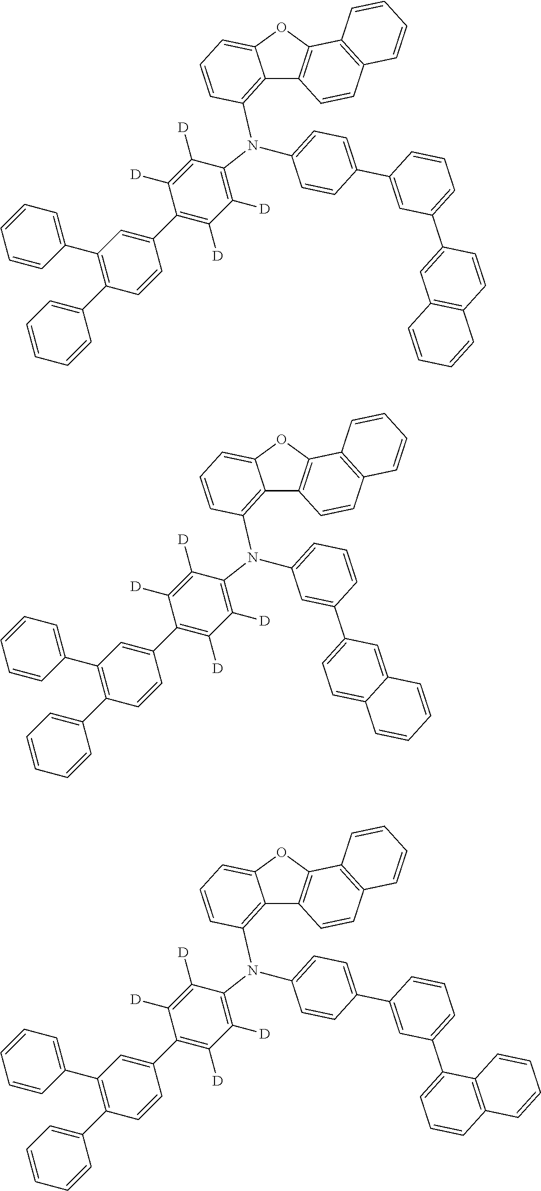 [SMILES: [2H]c1c([2H])c(N(c2ccc(-c3cccc(-c4ccc5ccccc5c4)c3)cc2)c2cccc3oc4c5ccccc5ccc4c23)c([2H])c([2H])c1-c1ccc(-c2ccccc2)c(-c2ccccc2)c1 ; [2H]c1c([2H])c(N(c2cccc(-c3ccc4ccccc4c3)c2)c2cccc3oc4c5ccccc5ccc4c23)c([2H])c([2H])c1-c1ccc(-c2ccccc2)c(-c2ccccc2)c1 ; [2H]c1c([2H])c(N(c2ccc(-c3cccc(-c4cccc5ccccc45)c3)cc2)c2cccc3oc4c5ccccc5ccc4c23)c([2H])c([2H])c1-c1ccc(-c2ccccc2)c(-c2ccccc2)c1]

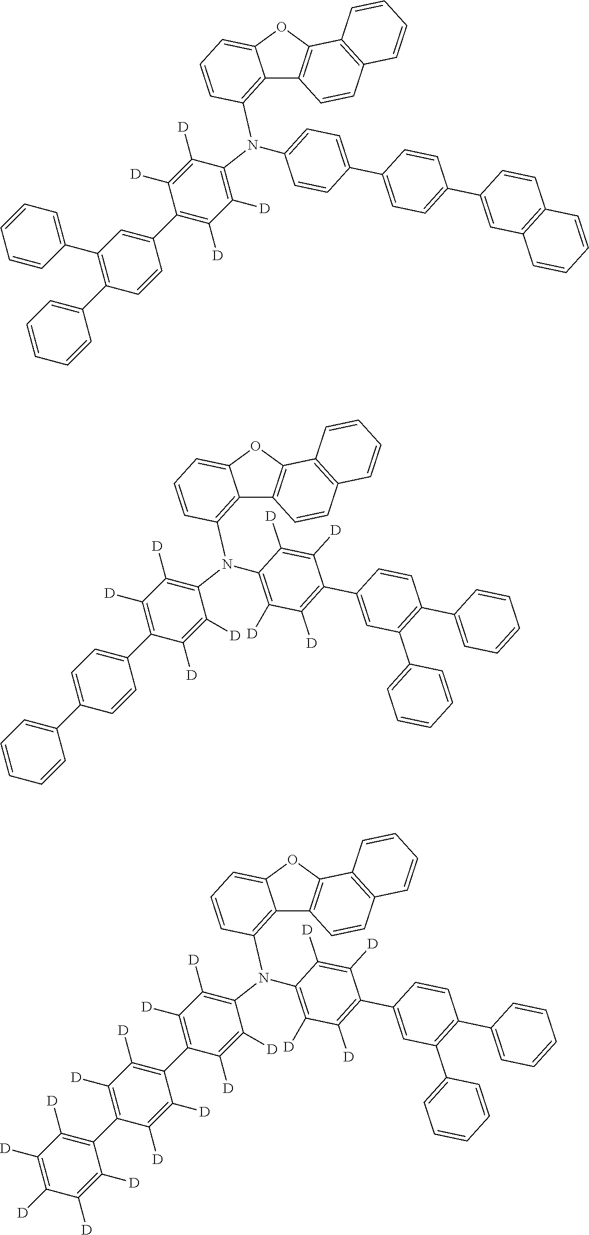 [SMILES: [2H]c1c([2H])c(N(c2ccc(-c3ccc(-c4ccc5ccccc5c4)cc3)cc2)c2cccc3oc4c5ccccc5ccc4c23)c([2H])c([2H])c1-c1ccc(-c2ccccc2)c(-c2ccccc2)c1 ; [2H]c1c([2H])c(N(c2c([2H])c([2H])c(-c3ccc(-c4ccccc4)c(-c4ccccc4)c3)c([2H])c2[2H])c2cccc3oc4c5ccccc5ccc4c23)c([2H])c([2H])c1-c1ccc(-c2ccccc2)cc1 ; [2H]c1c([2H])c([2H])c(-c2c([2H])c([2H])c(-c3c([2H])c([2H])c(N(c4c([2H])c([2H])c(-c5ccc(-c6ccccc6)c(-c6ccccc6)c5)c([2H])c4[2H])c4cccc5oc6c7ccccc7ccc6c45)c([2H])c3[2H])c([2H])c2[2H])c([2H])c1[2H]]

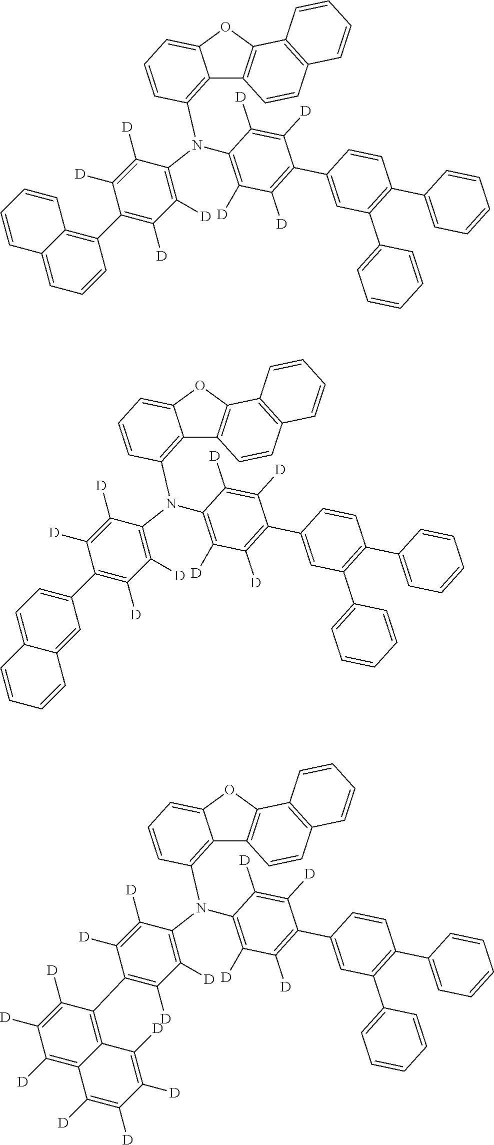 [SMILES: [2H]c1c([2H])c(N(c2c([2H])c([2H])c(-c3cccc4ccccc34)c([2H])c2[2H])c2cccc3oc4c5ccccc5ccc4c23)c([2H])c([2H])c1-c1ccc(-c2ccccc2)c(-c2ccccc2)c1 ; [2H]c1c([2H])c(N(c2c([2H])c([2H])c(-c3ccc4ccccc4c3)c([2H])c2[2H])c2cccc3oc4c5ccccc5ccc4c23)c([2H])c([2H])c1-c1ccc(-c2ccccc2)c(-c2ccccc2)c1 ; [2H]c1c([2H])c(N(c2c([2H])c([2H])c(-c3c([2H])c([2H])c([2H])c4c([2H])c([2H])c([2H])c([2H])c34)c([2H])c2[2H])c2cccc3oc4c5ccccc5ccc4c23)c([2H])c([2H])c1-c1ccc(-c2ccccc2)c(-c2ccccc2)c1]

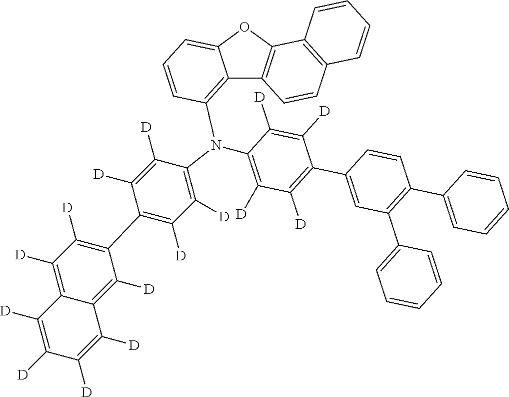 [SMILES: [2H]c1c([2H])c(N(c2c([2H])c([2H])c(-c3c([2H])c([2H])c4c([2H])c([2H])c([2H])c([2H])c4c3[2H])c([2H])c2[2H])c2cccc3oc4c5ccccc5ccc4c23)c([2H])c([2H])c1-c1ccc(-c2ccccc2)c(-c2ccccc2)c1]

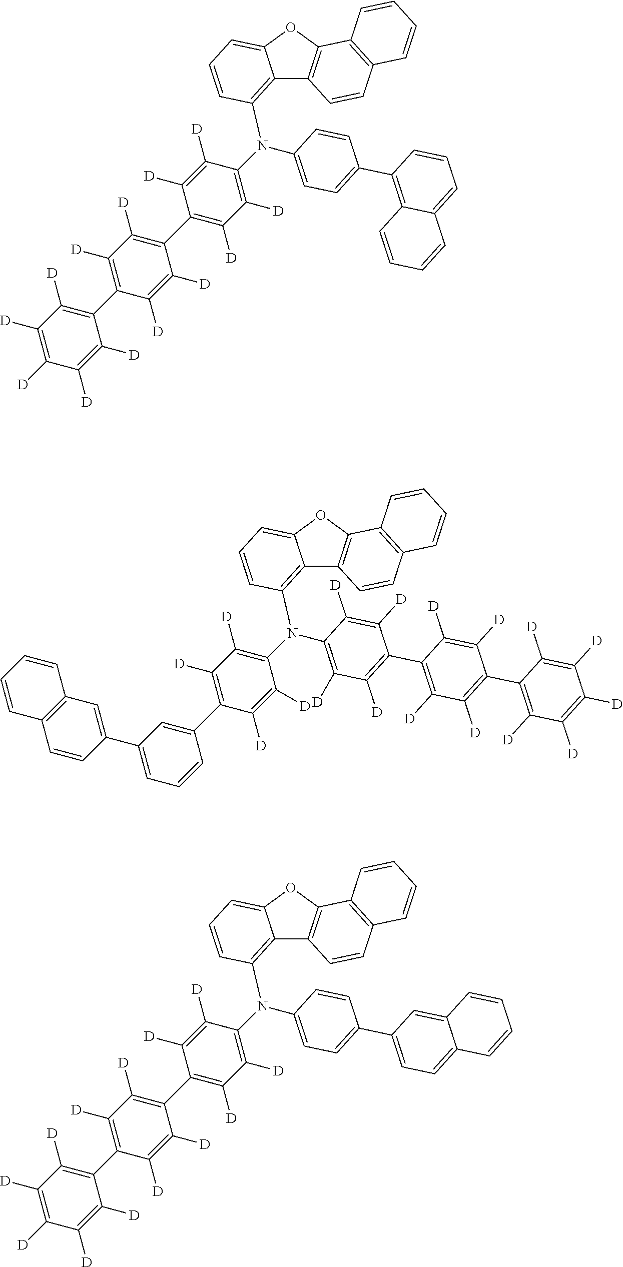

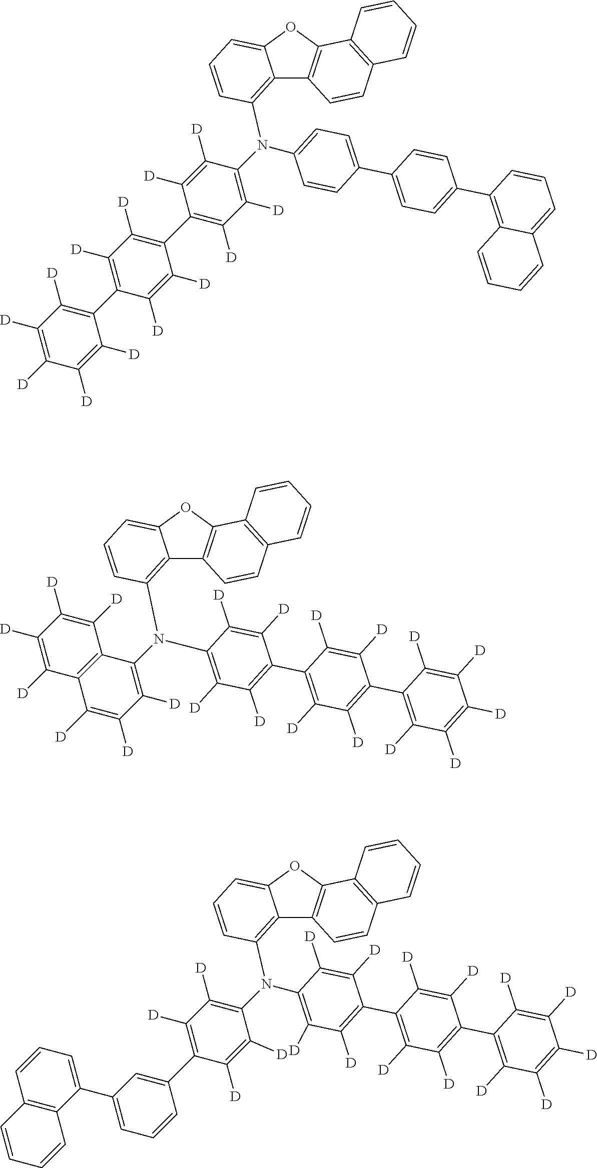

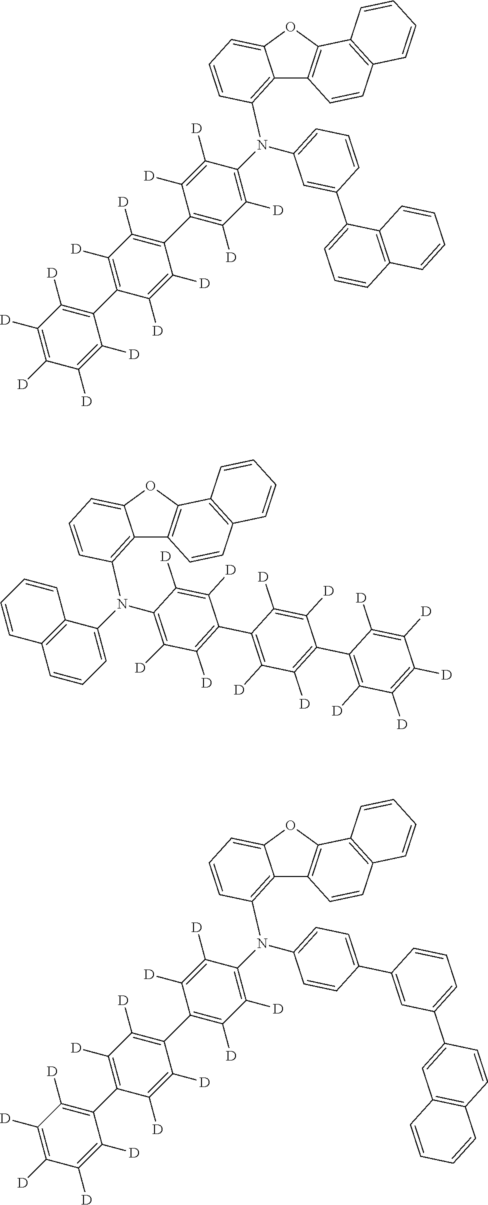 [SMILES: [2H]c1c([2H])c([2H])c(-c2c([2H])c([2H])c(-c3c([2H])c([2H])c(N(c4cccc(-c5cccc6ccccc56)c4)c4cccc5oc6c7ccccc7ccc6c45)c([2H])c3[2H])c([2H])c2[2H])c([2H])c1[2H] ; [2H]c1c([2H])c([2H])c(-c2c([2H])c([2H])c(-c3c([2H])c([2H])c(N(c4cccc5ccccc45)c4cccc5oc6c7ccccc7ccc6c45)c([2H])c3[2H])c([2H])c2[2H])c([2H])c1[2H] ; [2H]c1c([2H])c([2H])c(-c2c([2H])c([2H])c(-c3c([2H])c([2H])c(N(c4ccc(-c5cccc(-c6ccc7ccccc7c6)c5)cc4)c4cccc5oc6c7ccccc7ccc6c45)c([2H])c3[2H])c([2H])c2[2H])c([2H])c1[2H]]

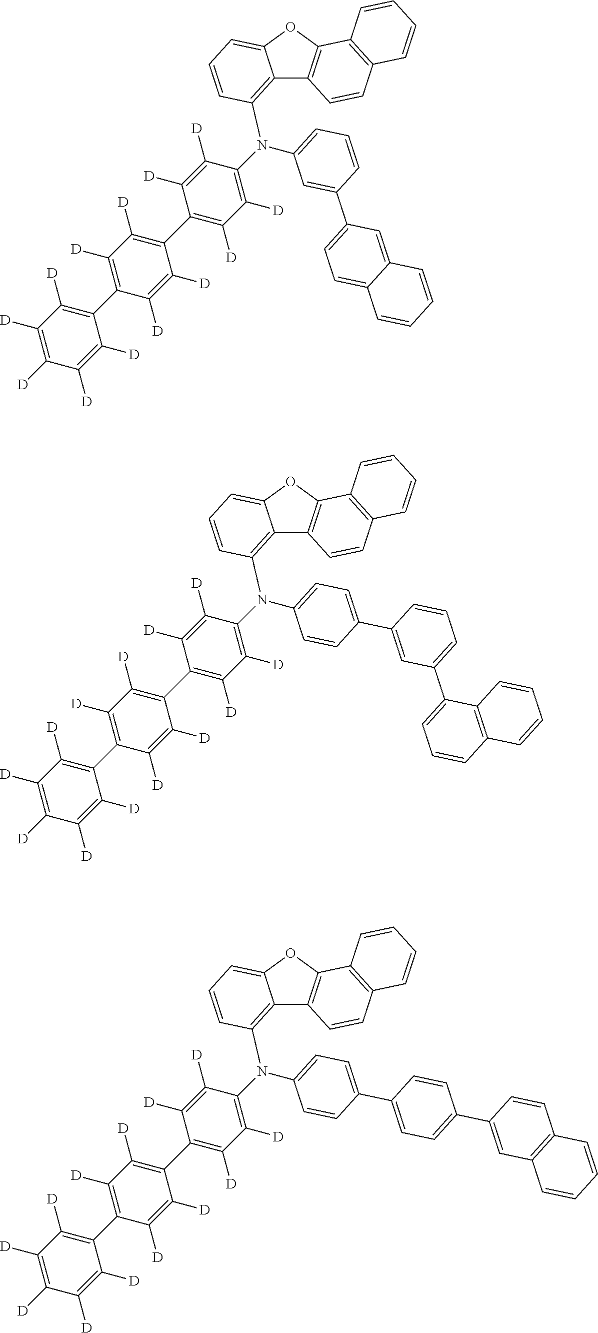 [SMILES: [2H]c1c([2H])c([2H])c(-c2c([2H])c([2H])c(-c3c([2H])c([2H])c(N(c4cccc(-c5ccc6ccccc6c5)c4)c4cccc5oc6c7ccccc7ccc6c45)c([2H])c3[2H])c([2H])c2[2H])c([2H])c1[2H] ; [2H]c1c([2H])c([2H])c(-c2c([2H])c([2H])c(-c3c([2H])c([2H])c(N(c4ccc(-c5cccc(-c6cccc7ccccc67)c5)cc4)c4cccc5oc6c7ccccc7ccc6c45)c([2H])c3[2H])c([2H])c2[2H])c([2H])c1[2H] ; [2H]c1c([2H])c([2H])c(-c2c([2H])c([2H])c(-c3c([2H])c([2H])c(N(c4ccc(-c5ccc(-c6ccc7ccccc7c6)cc5)cc4)c4cccc5oc6c7ccccc7ccc6c45)c([2H])c3[2H])c([2H])c2[2H])c([2H])c1[2H]]

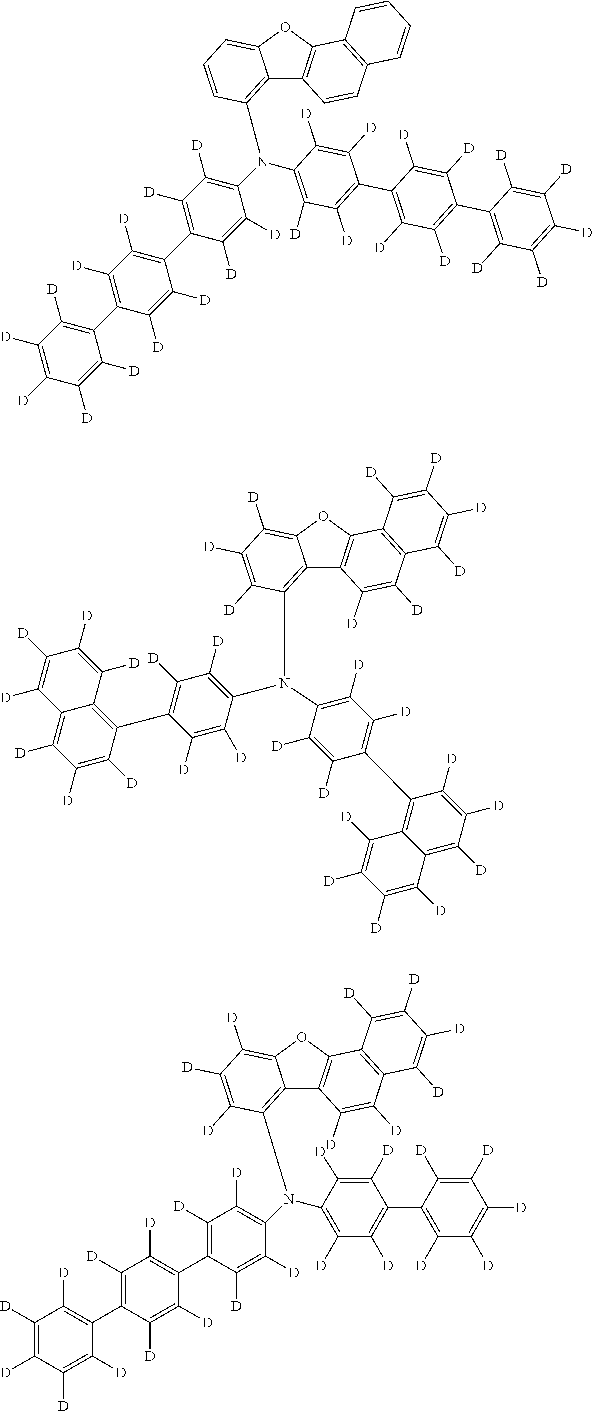

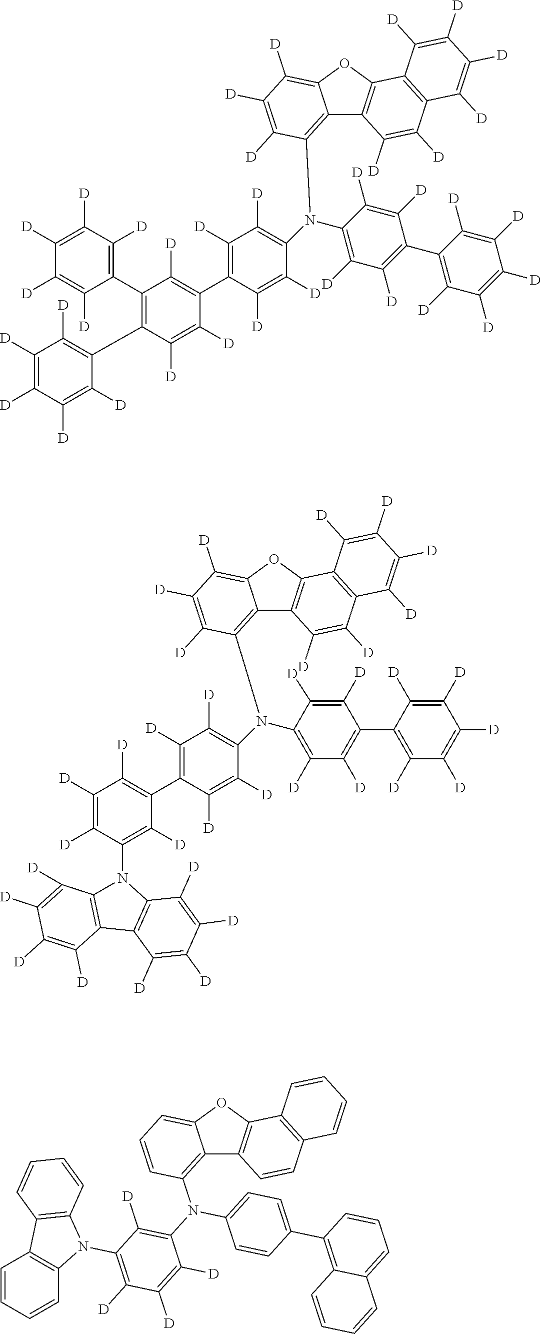

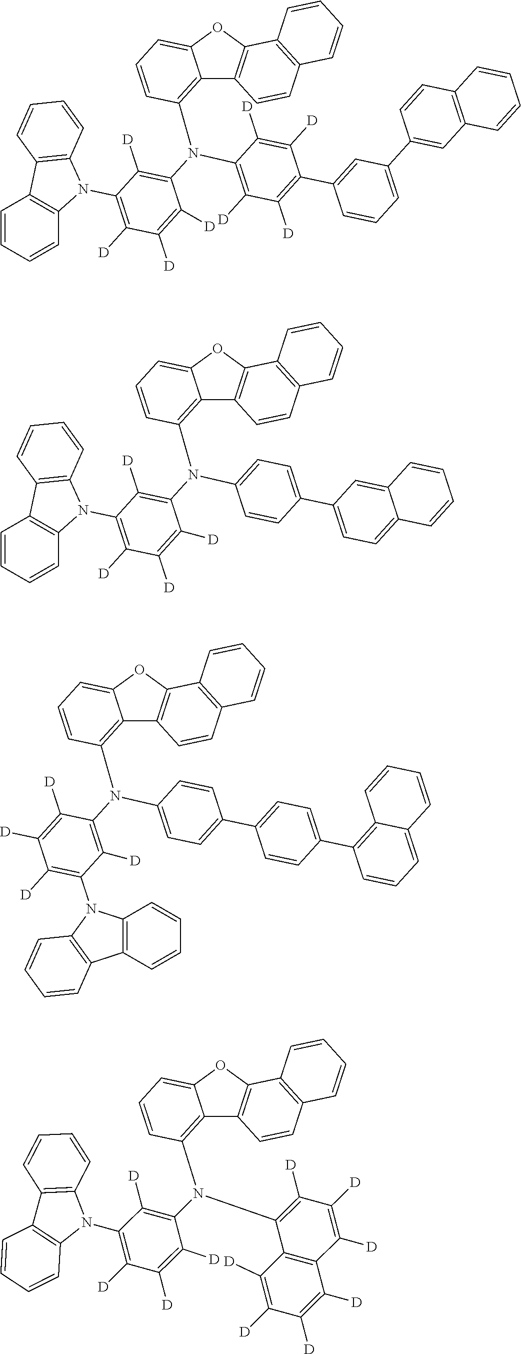

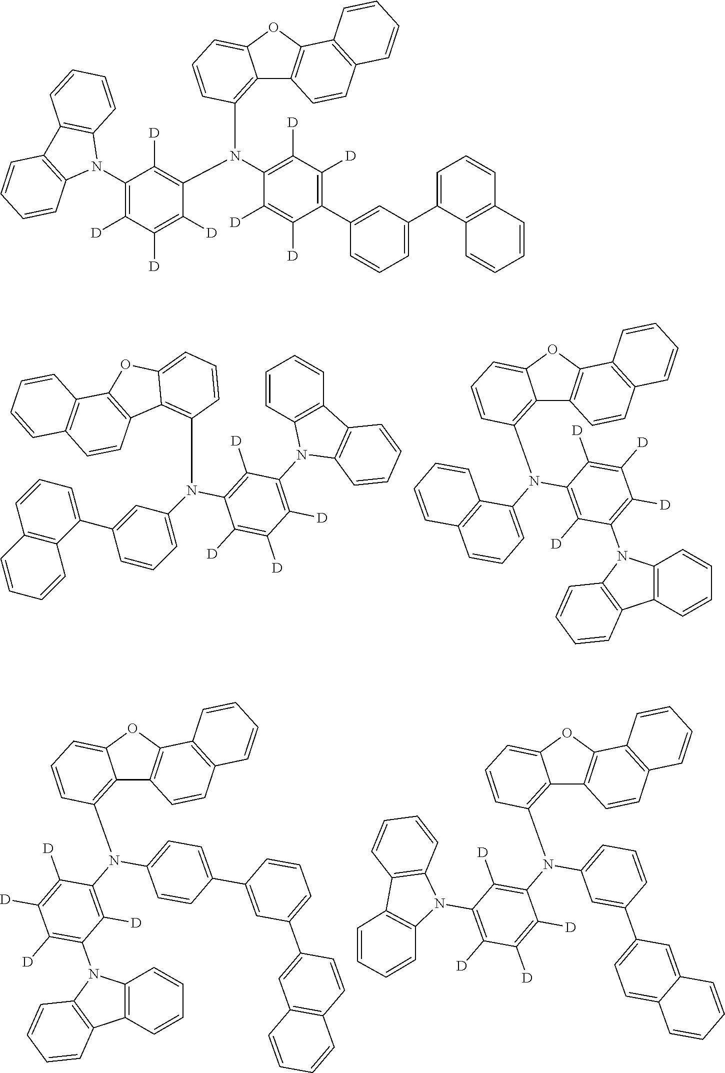

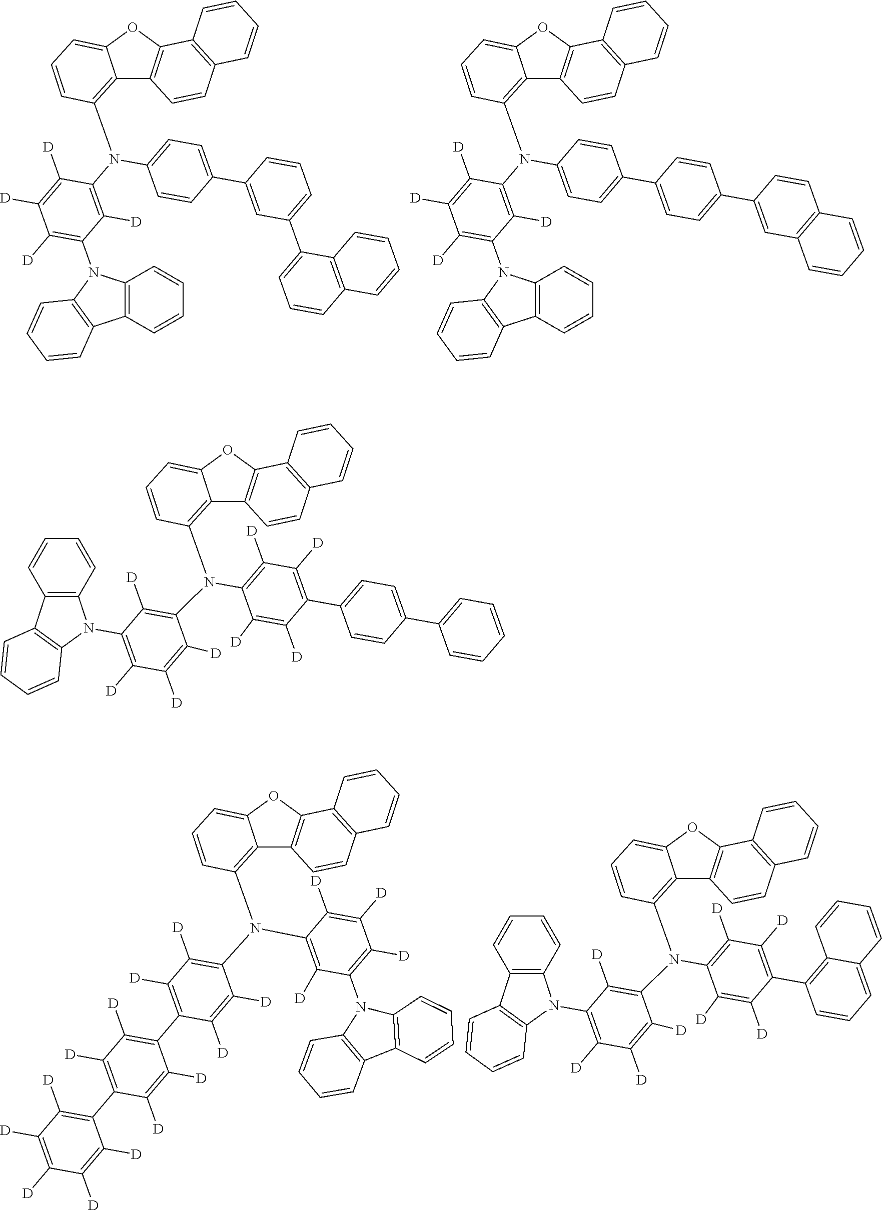

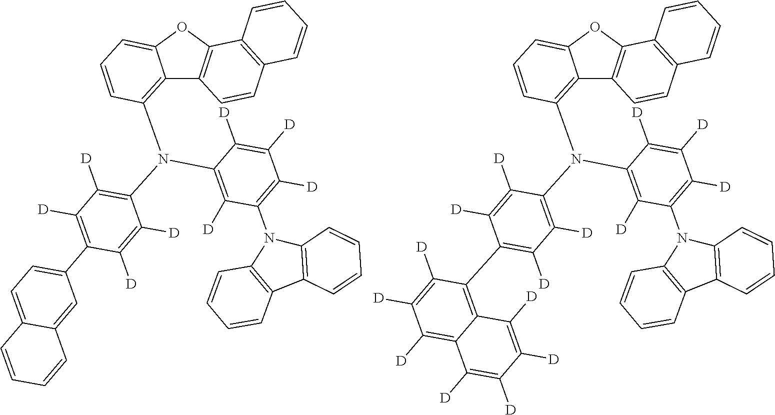

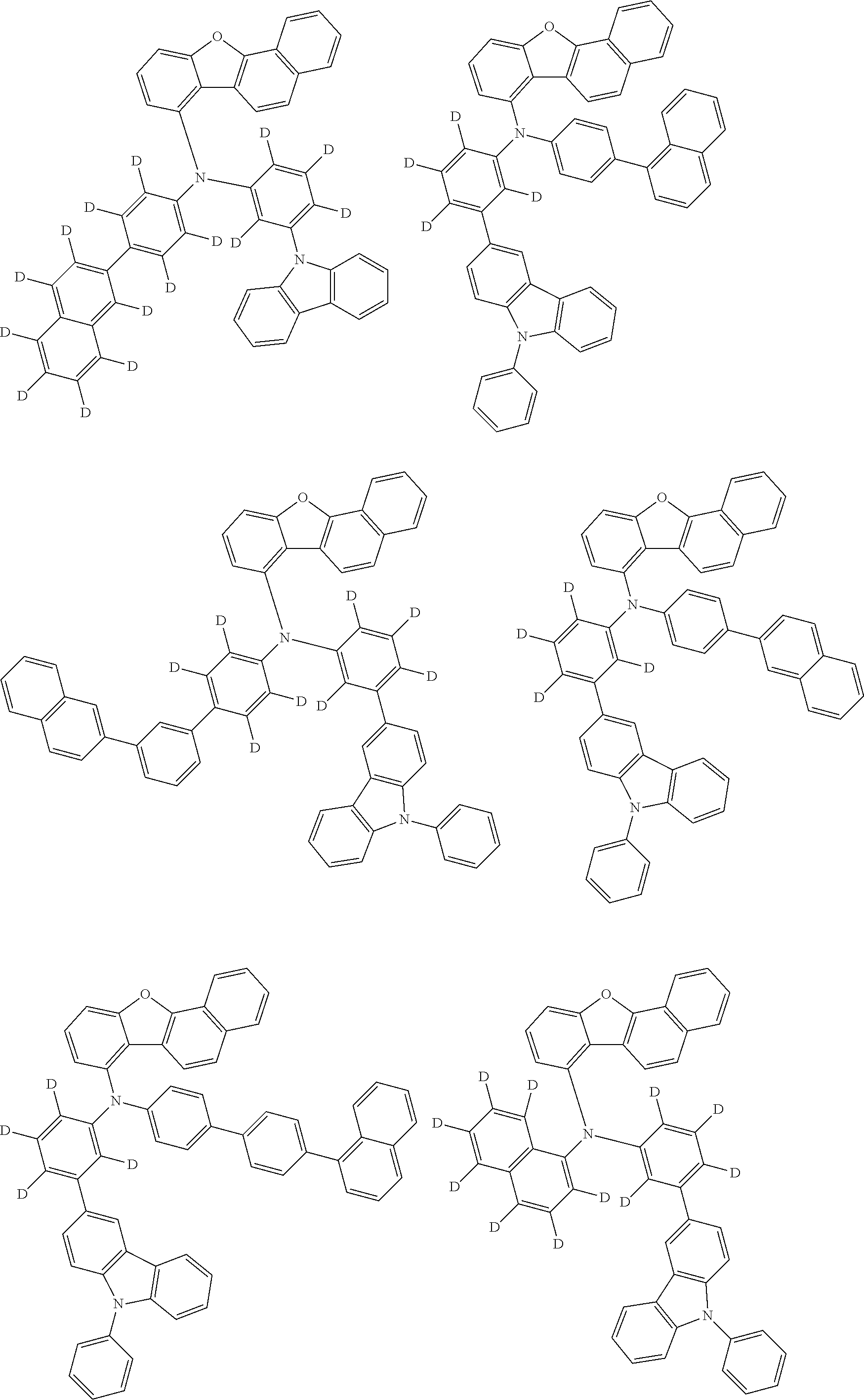

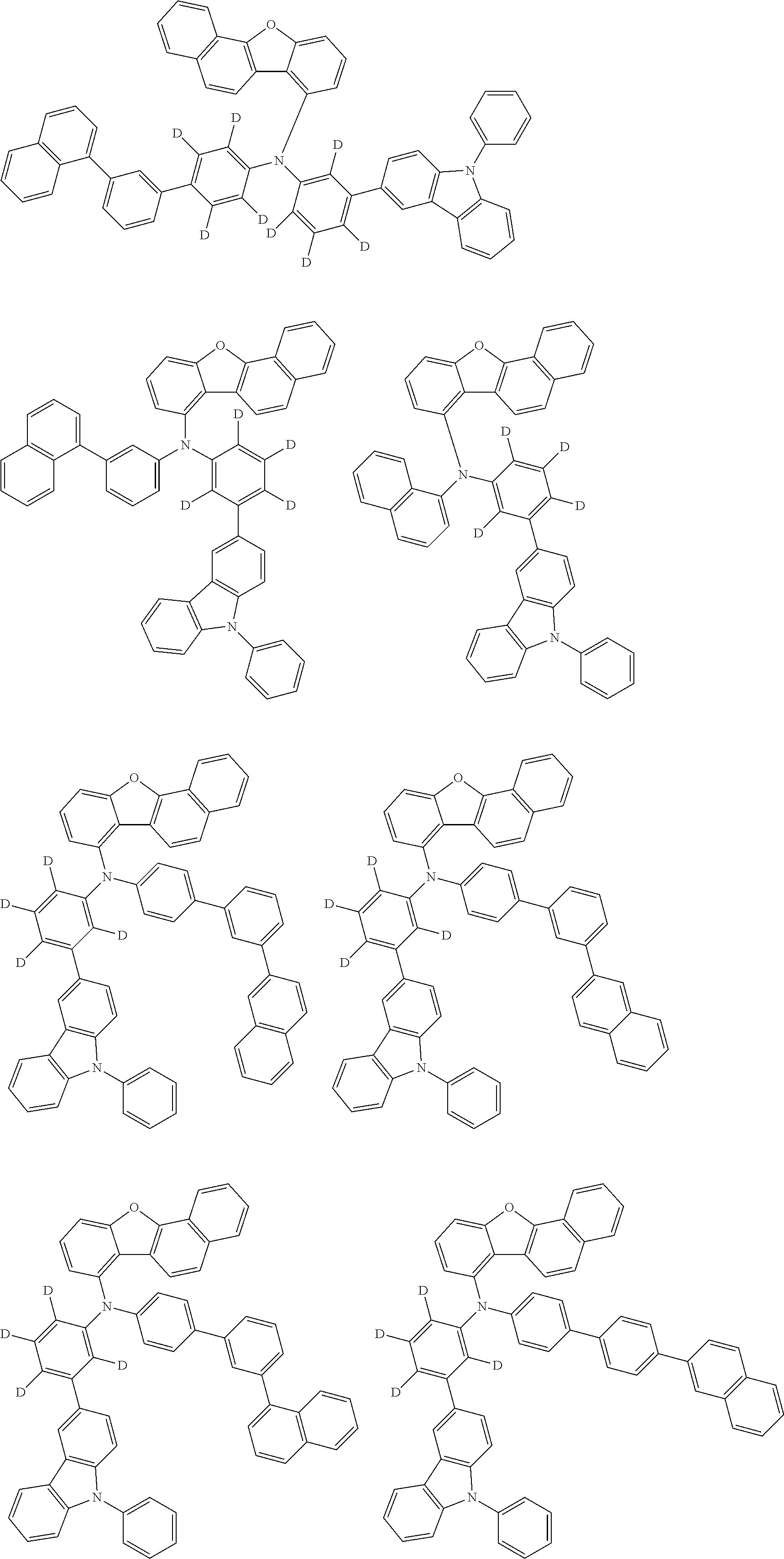

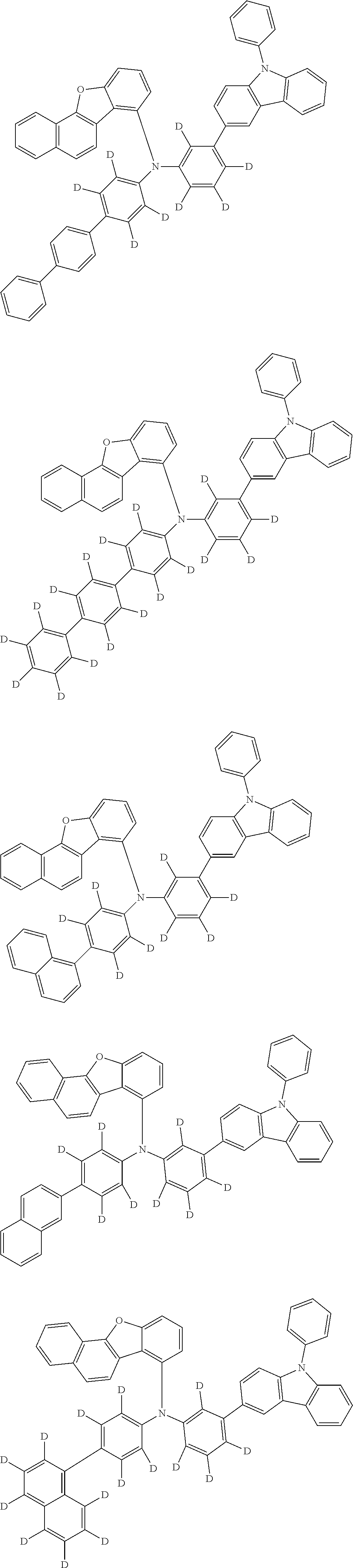

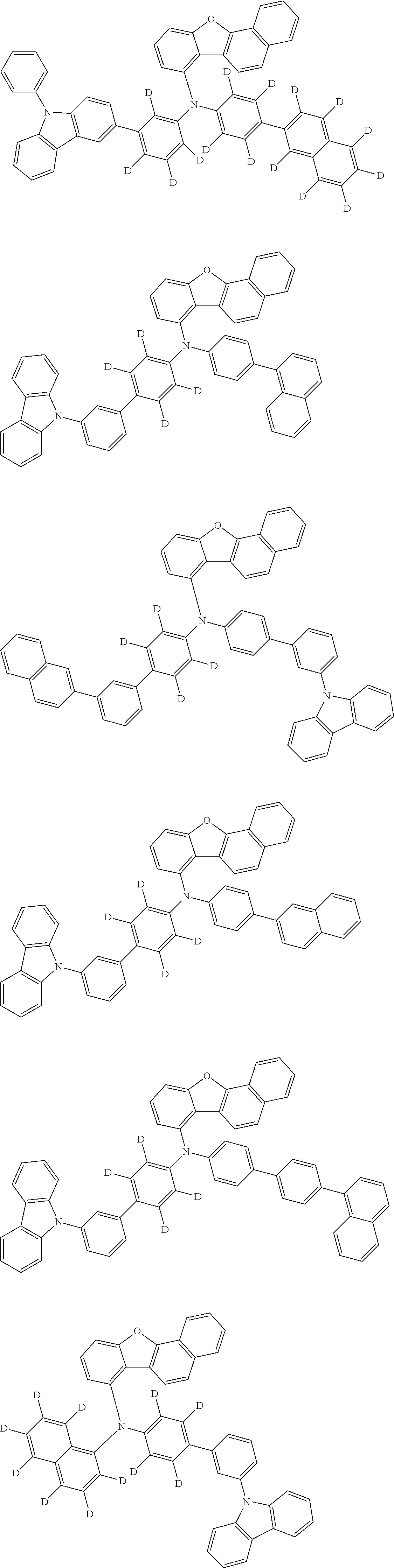

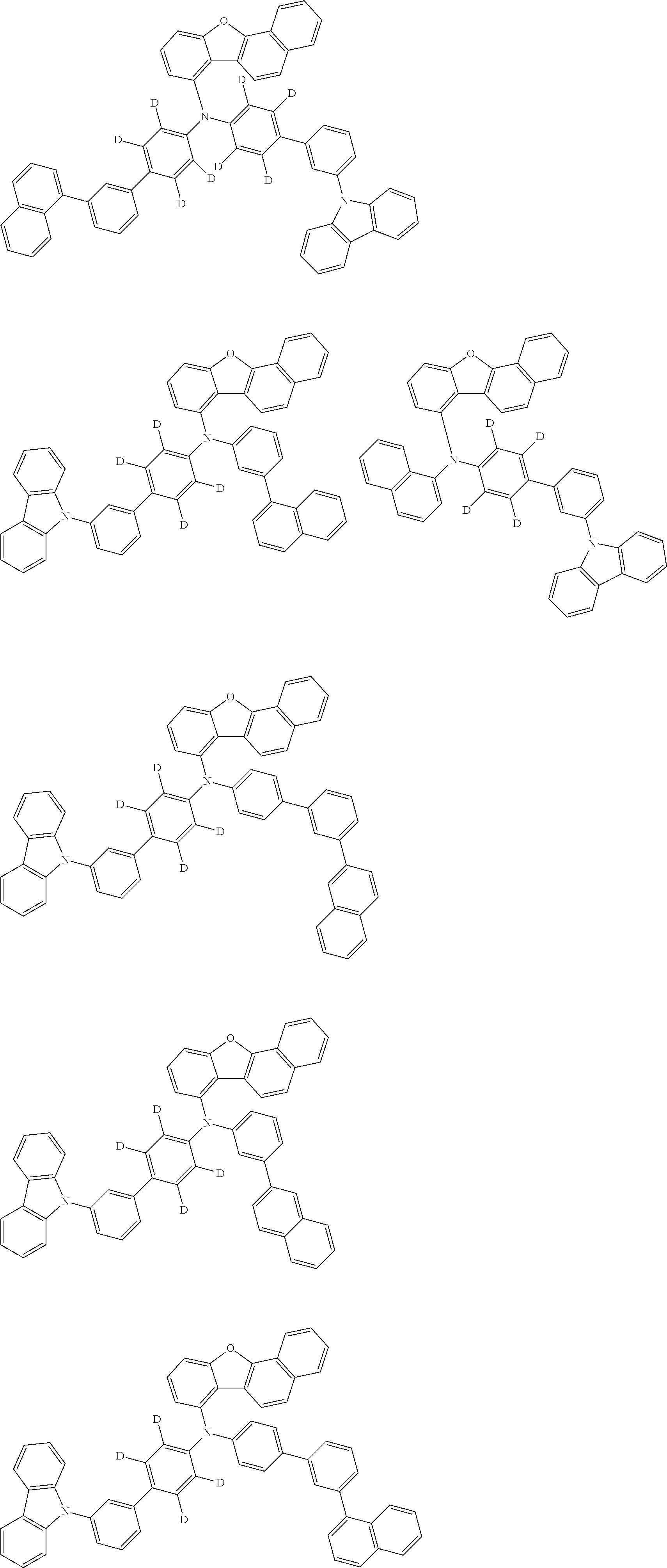

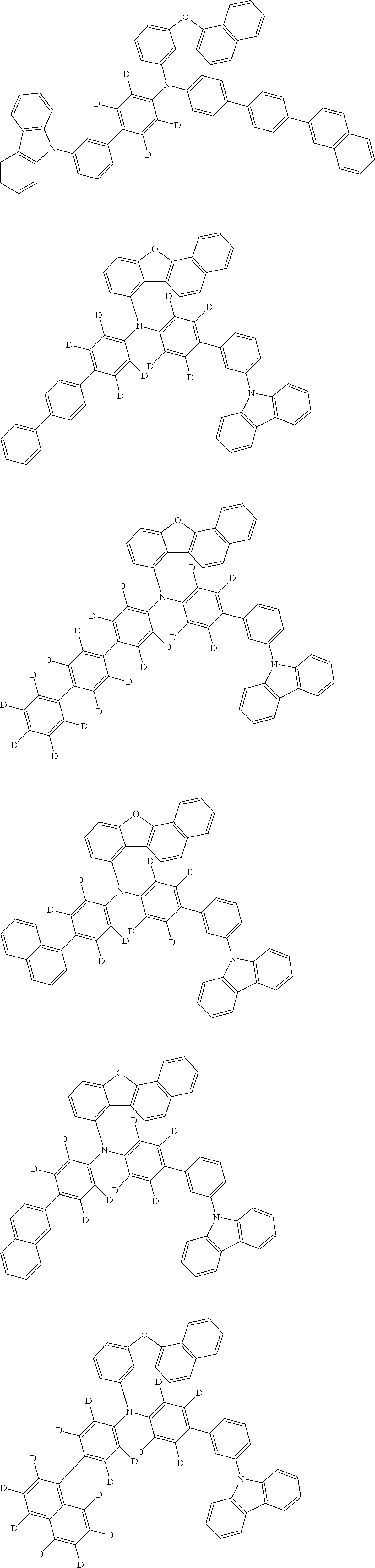

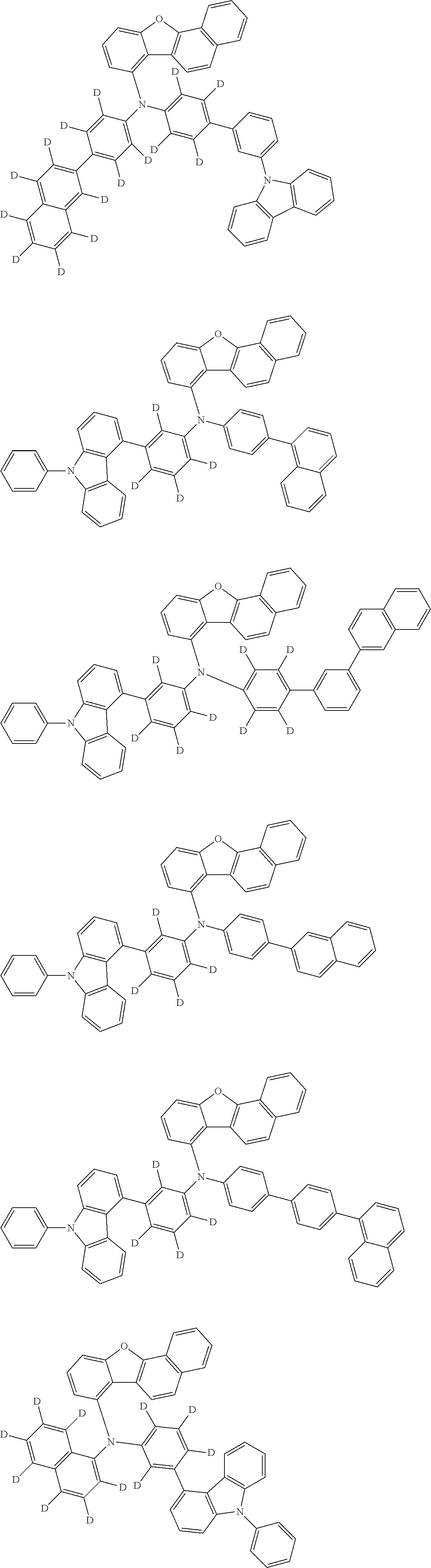

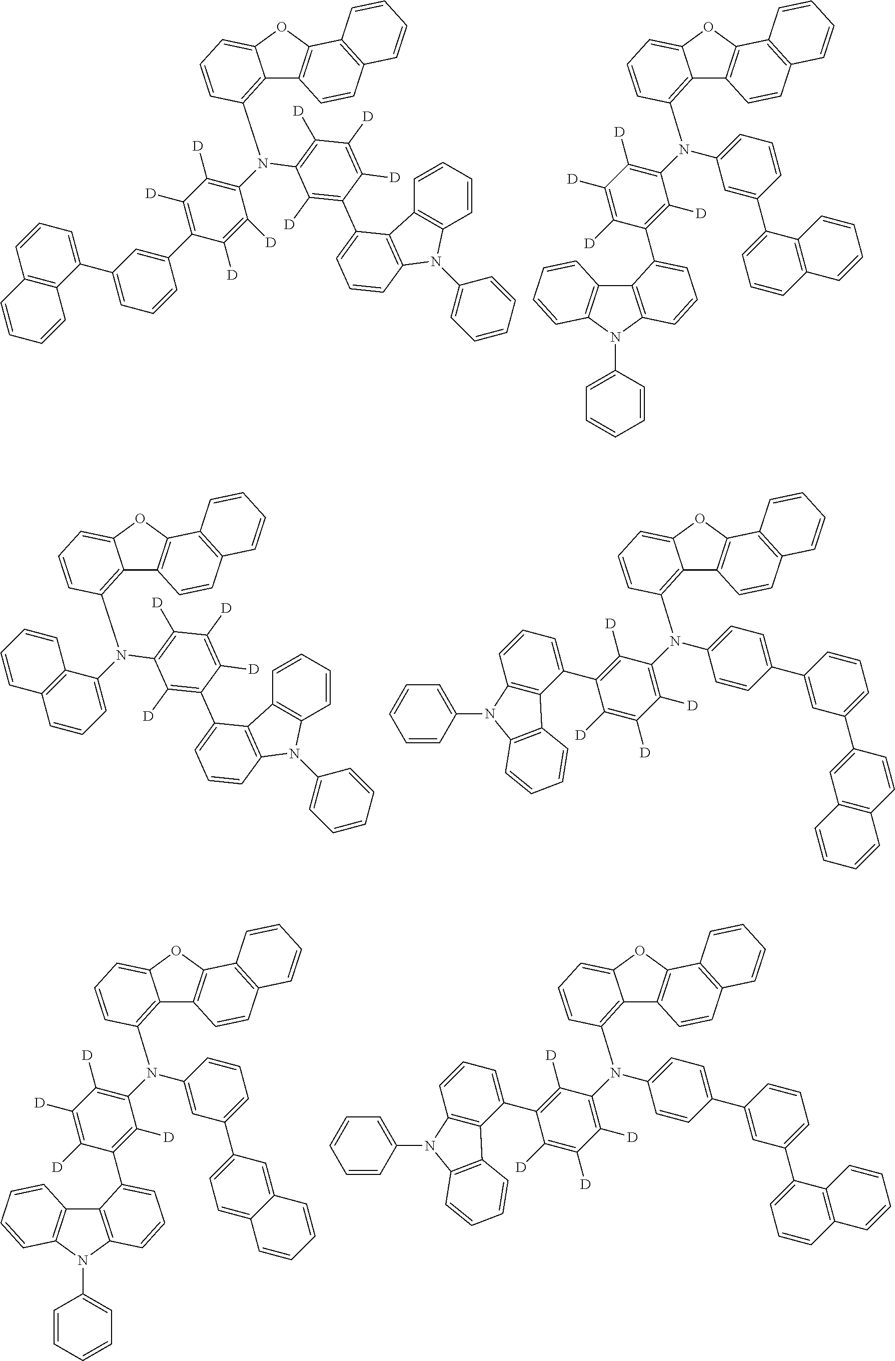

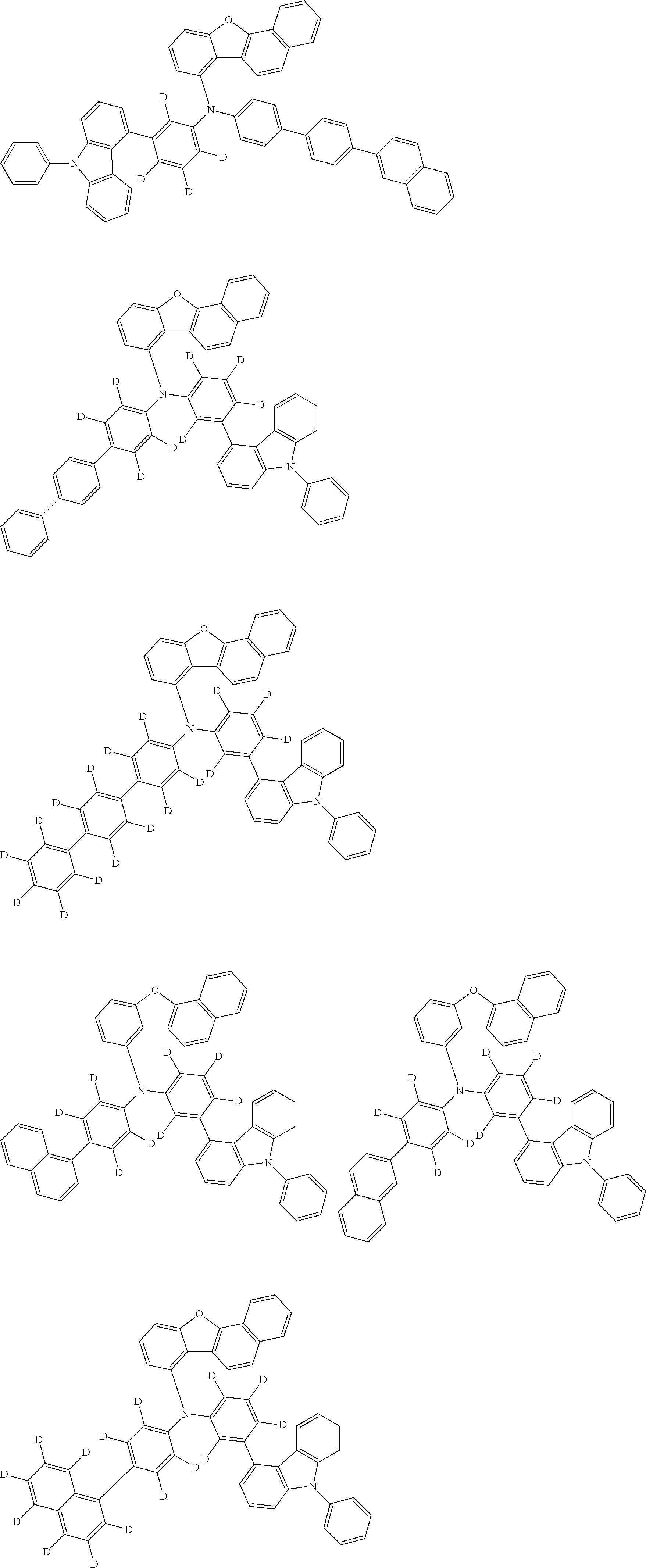

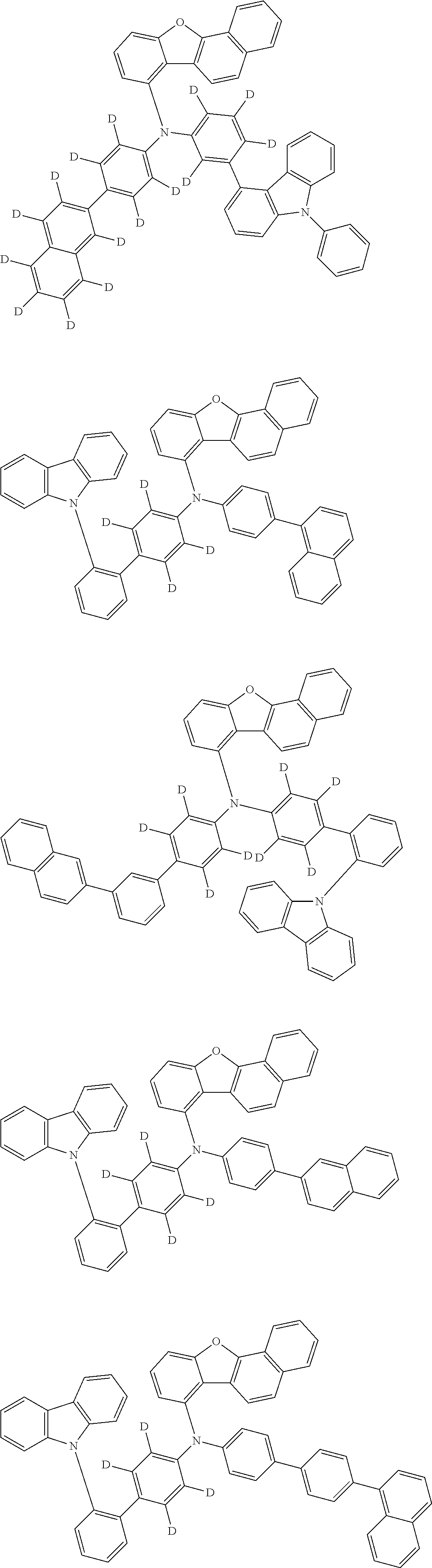

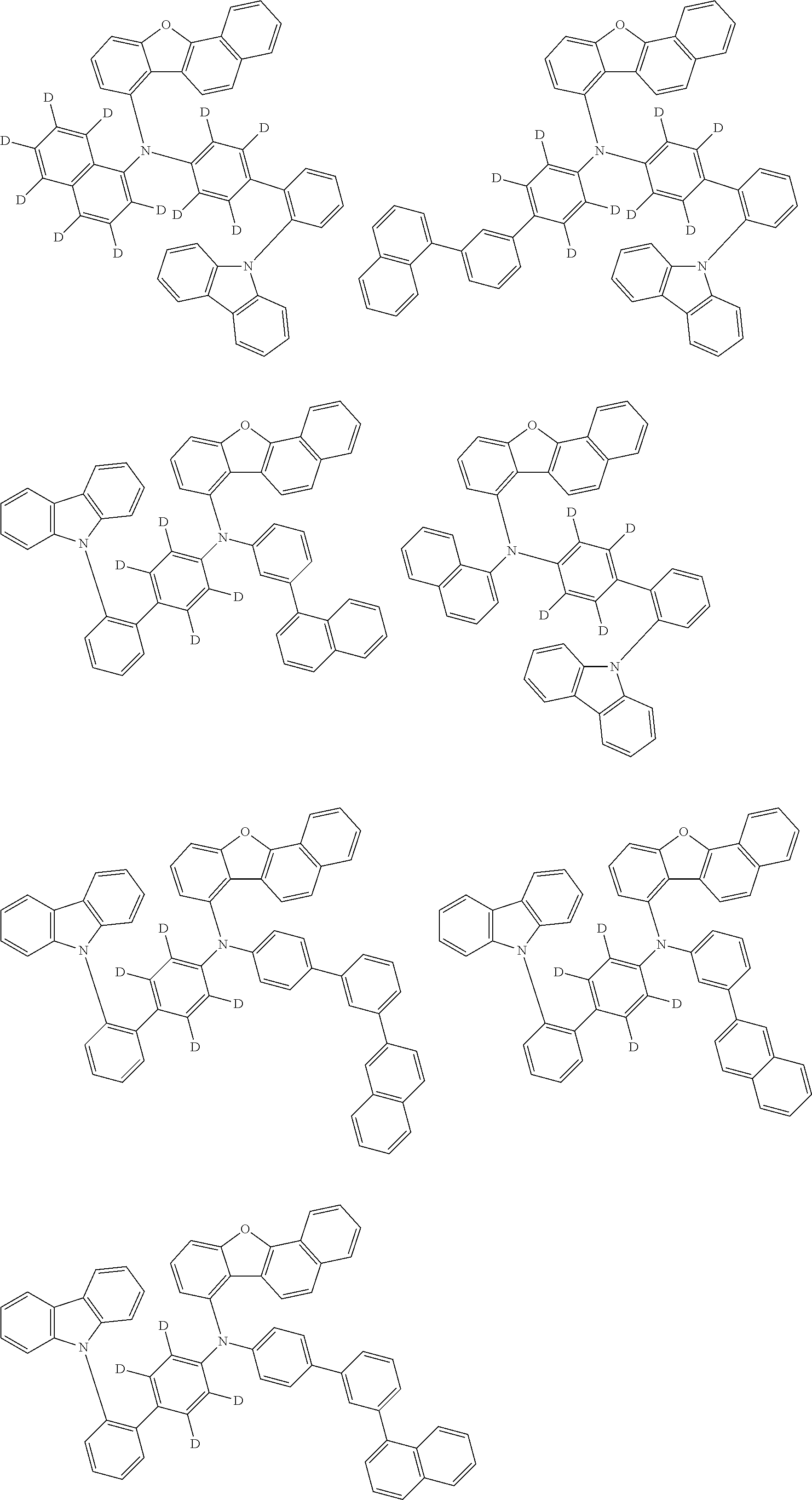

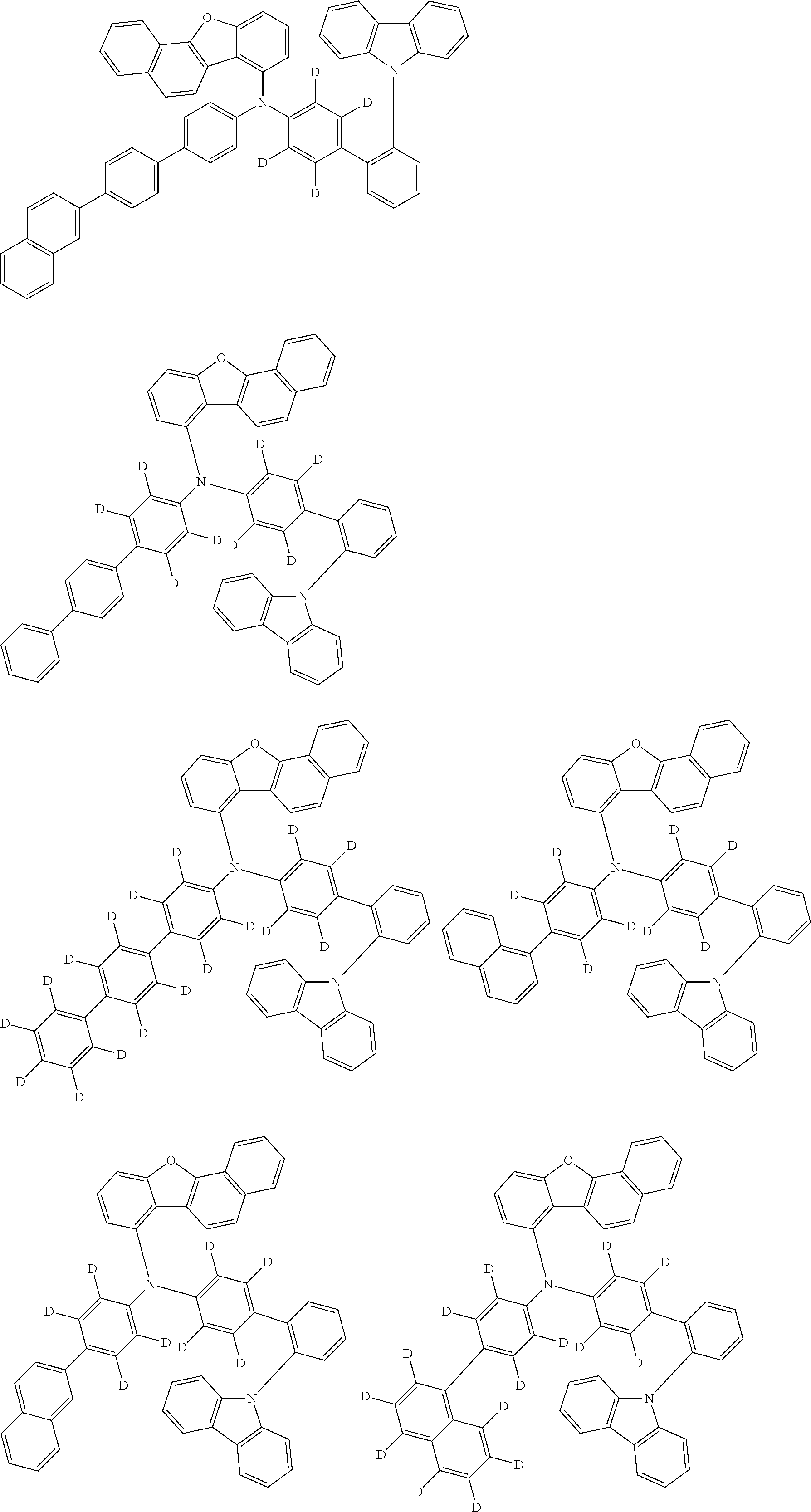

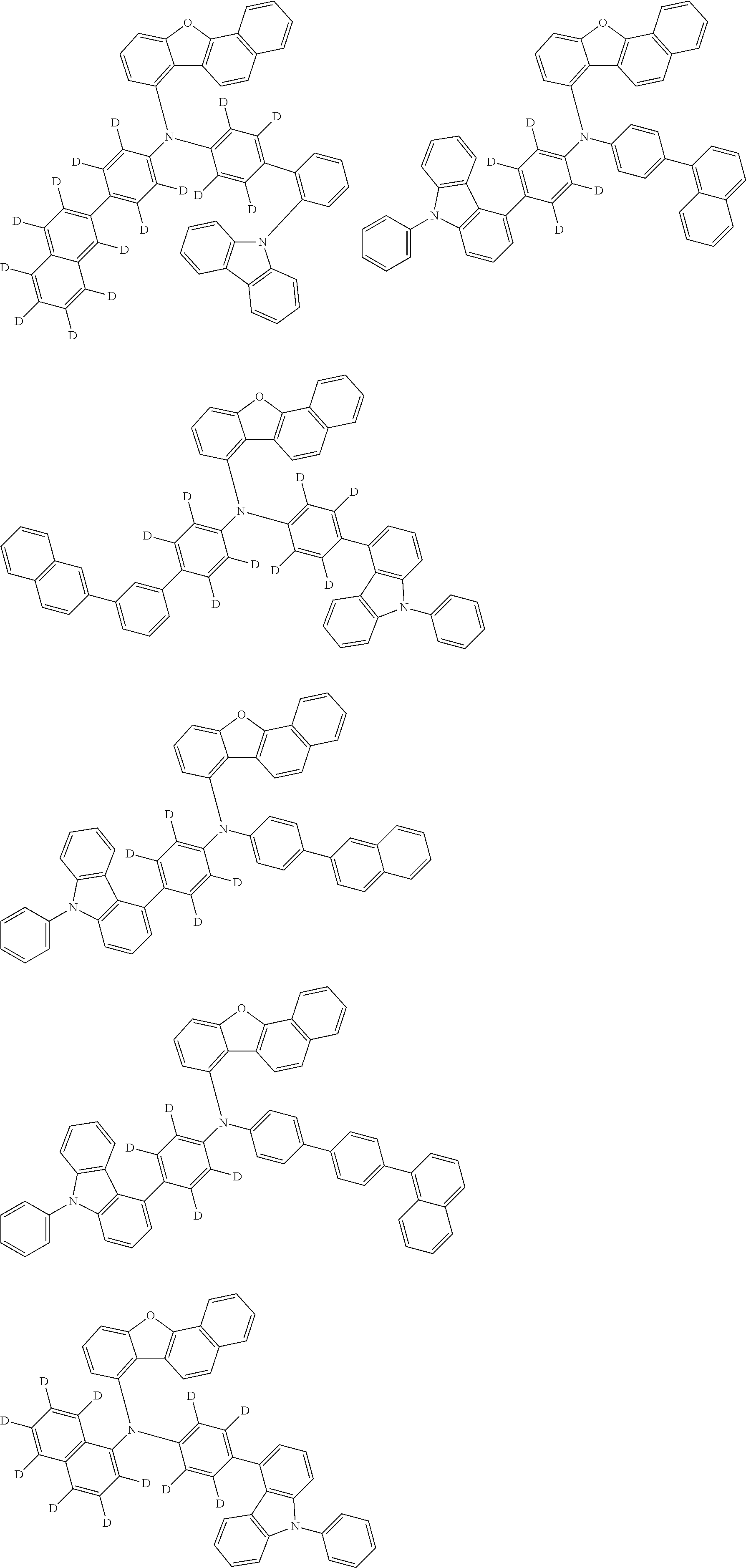

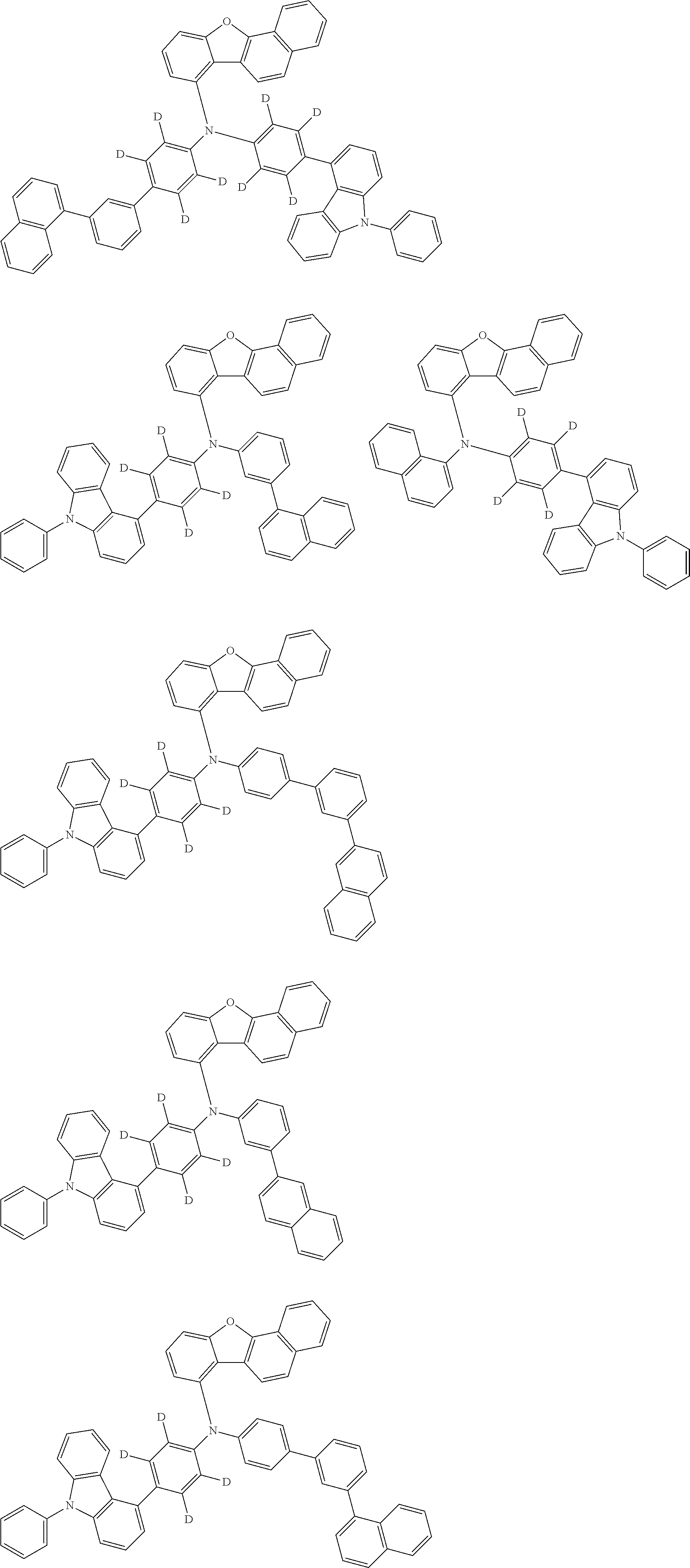

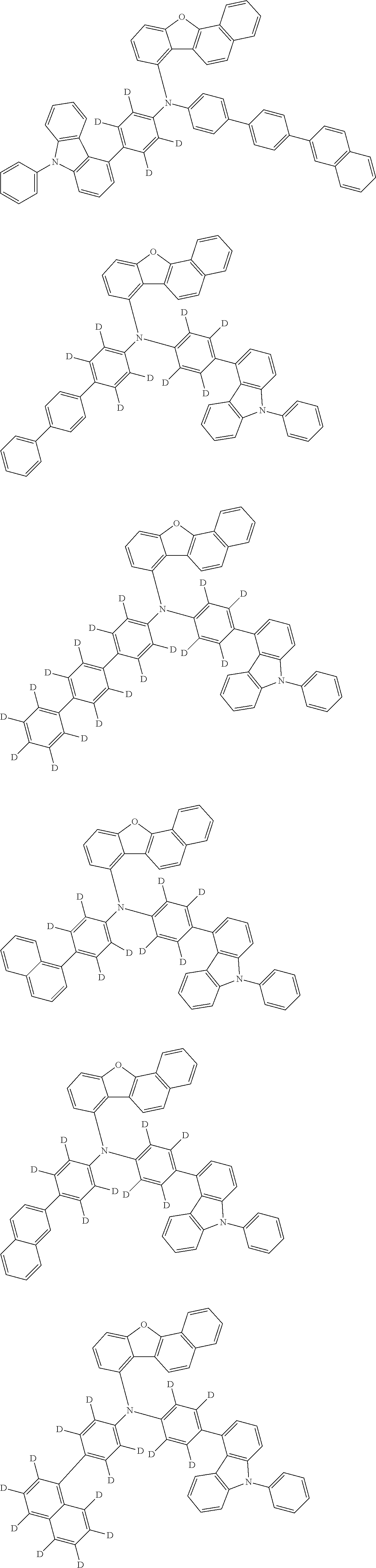

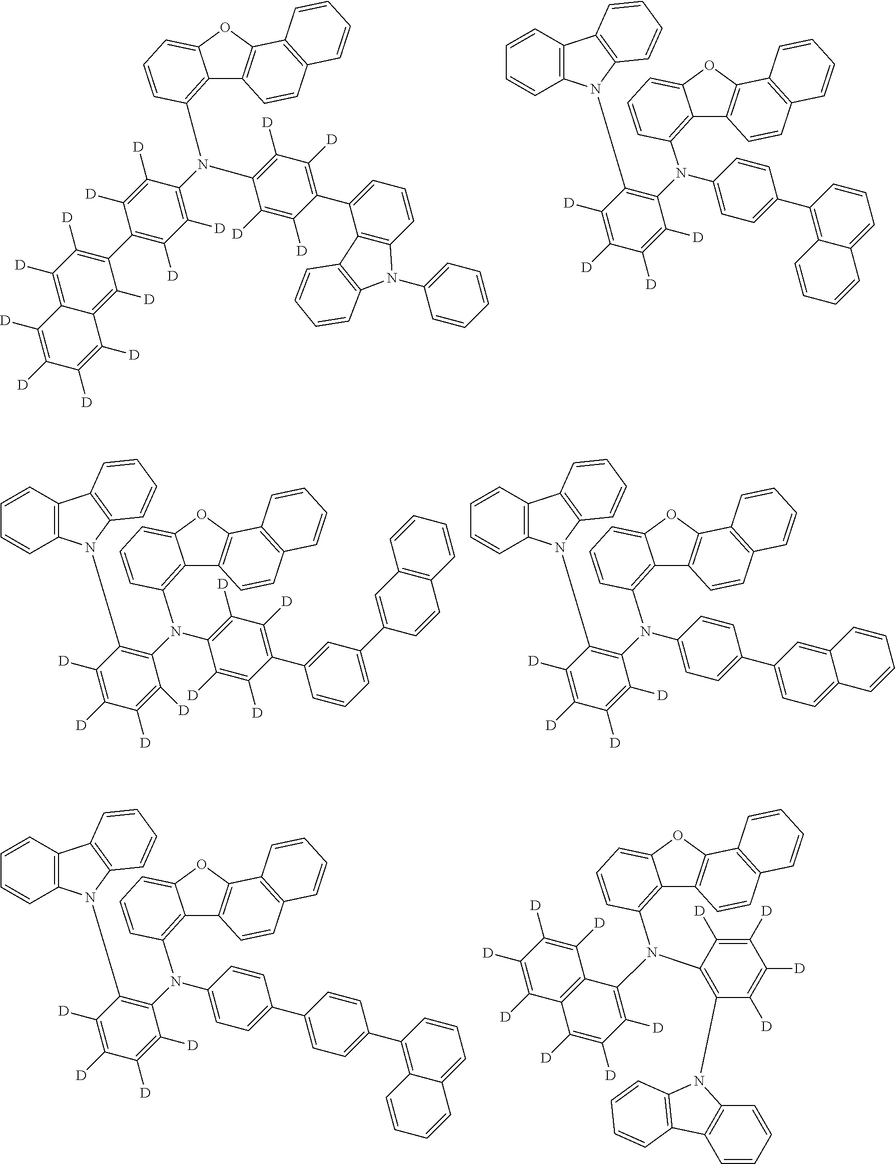

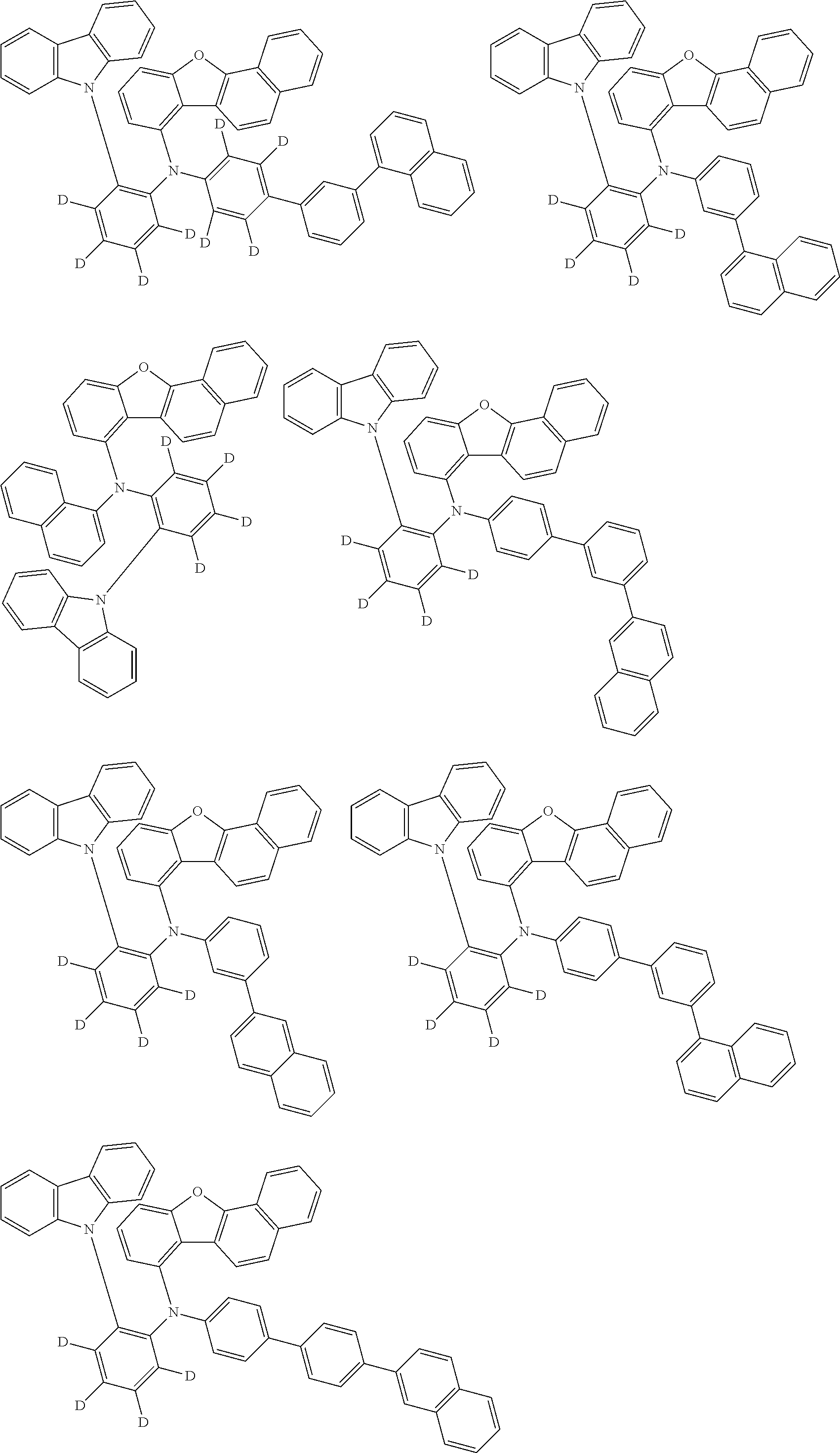

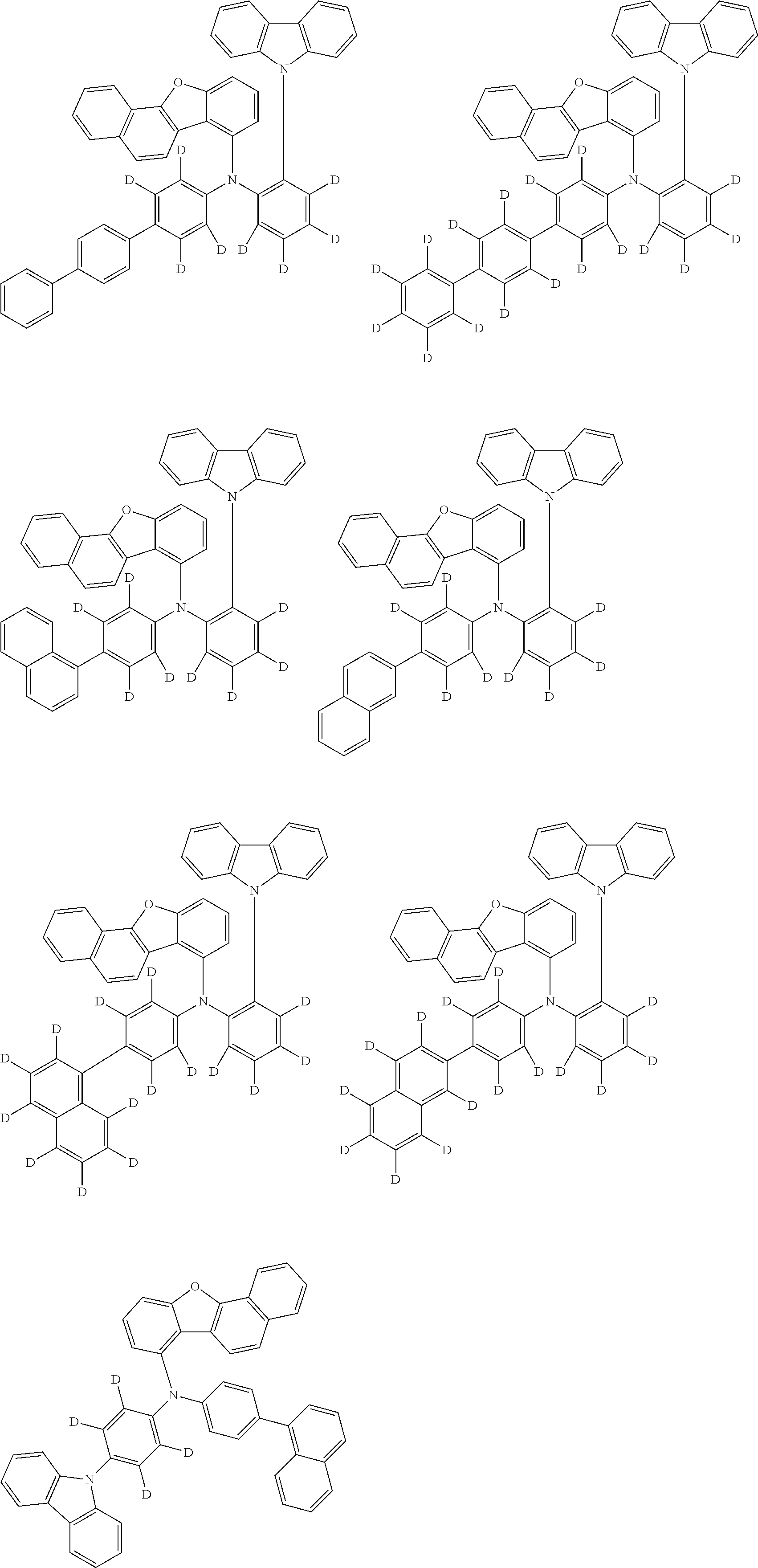

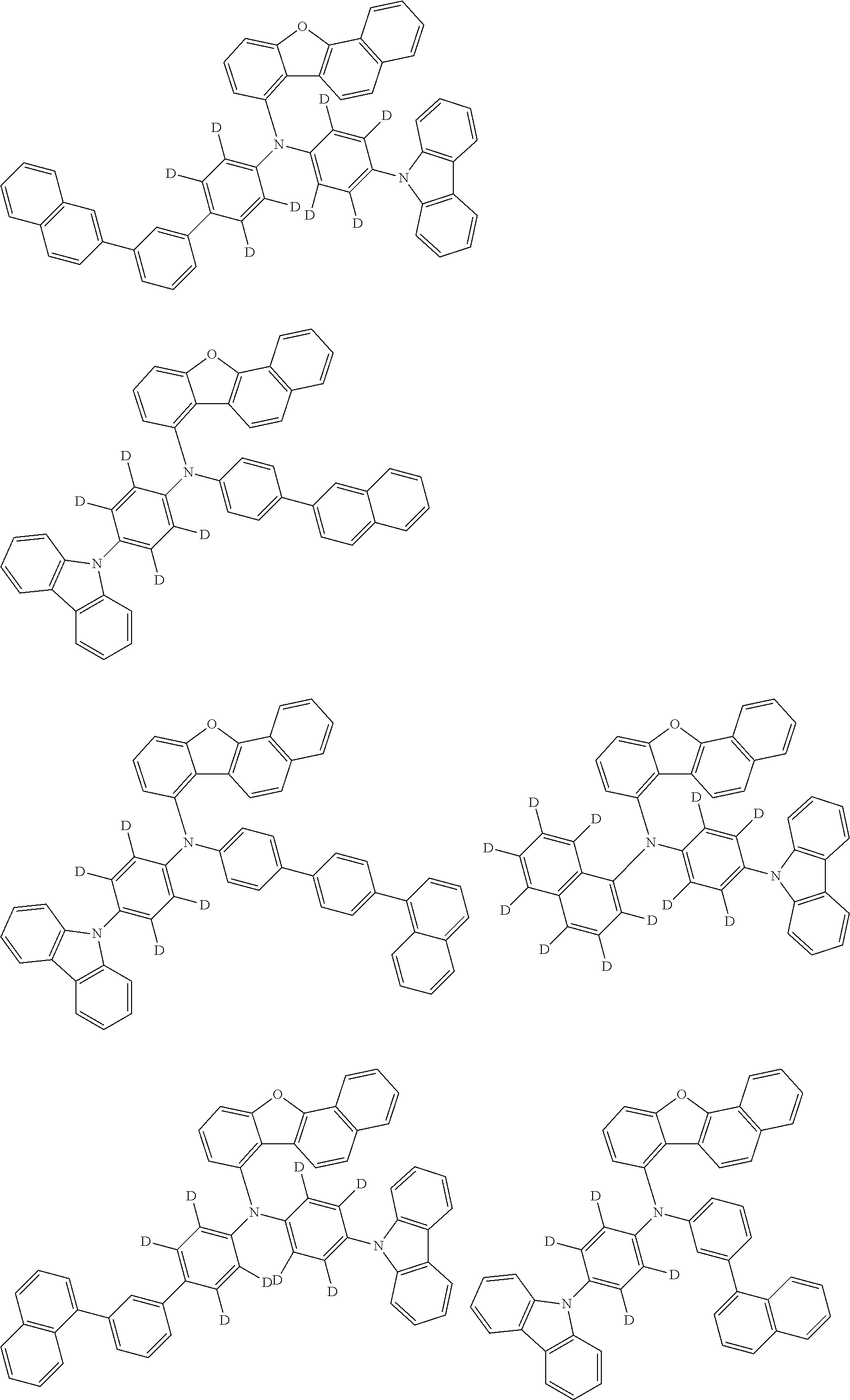

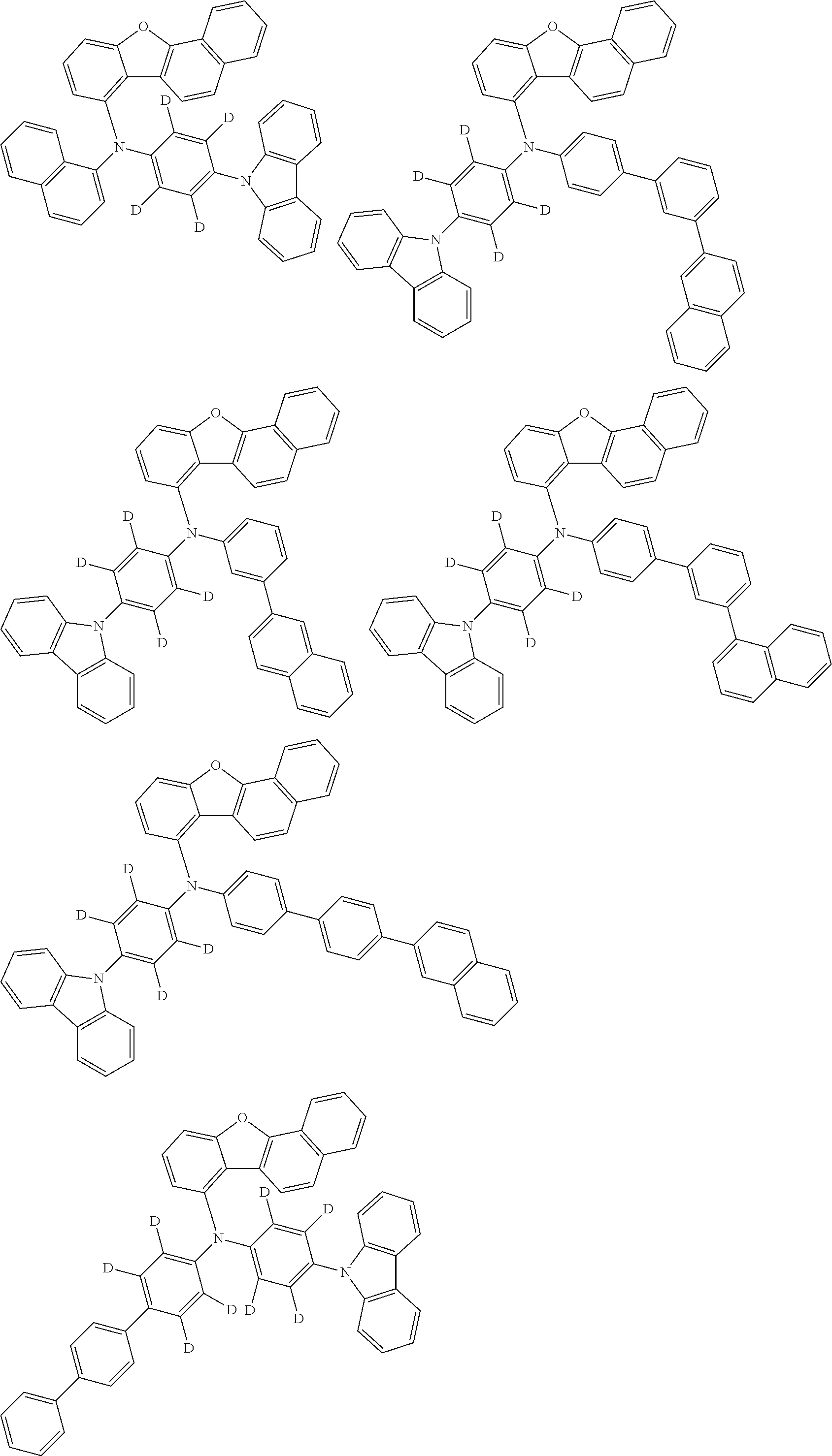

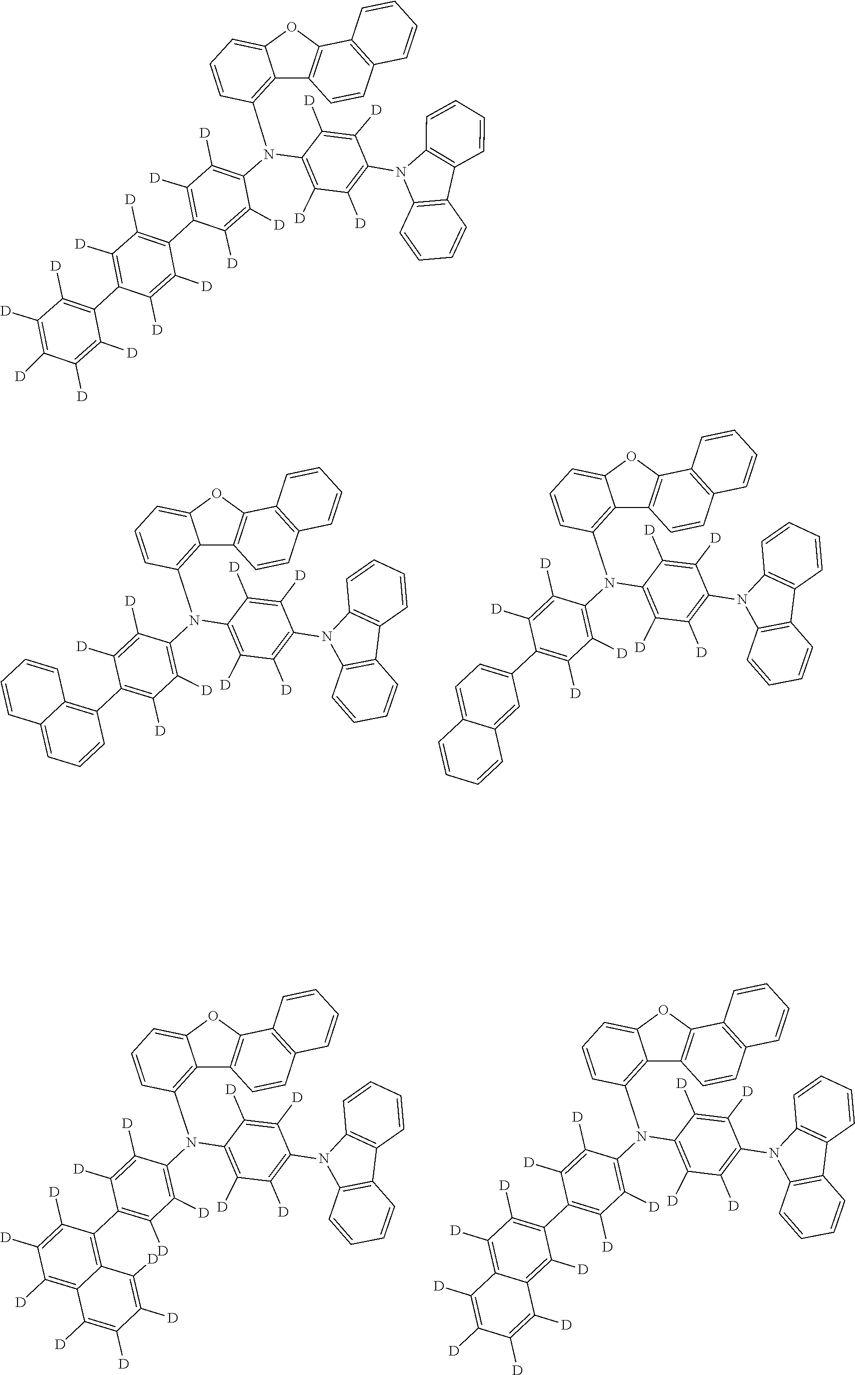

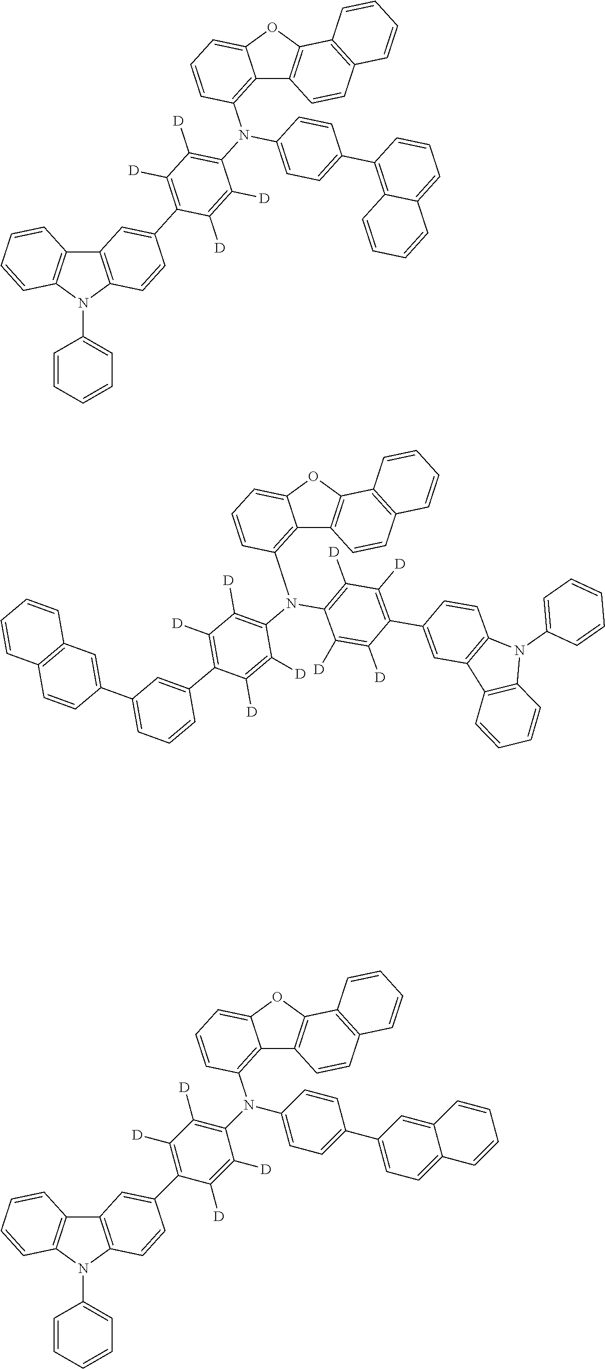 [SMILES: [2H]c1c([2H])c(N(c2ccc(-c3cccc4ccccc34)cc2)c2cccc3oc4c5ccccc5ccc4c23)c([2H])c([2H])c1-c1ccc2c(c1)c1ccccc1n2-c1ccccc1 ; [2H]c1c([2H])c(N(c2c([2H])c([2H])c(-c3ccc4c(c3)c3ccccc3n4-c3ccccc3)c([2H])c2[2H])c2cccc3oc4c5ccccc5ccc4c23)c([2H])c([2H])c1-c1cccc(-c2ccc3ccccc3c2)c1 ; [2H]c1c([2H])c(N(c2ccc(-c3ccc4ccccc4c3)cc2)c2cccc3oc4c5ccccc5ccc4c23)c([2H])c([2H])c1-c1ccc2c(c1)c1ccccc1n2-c1ccccc1]

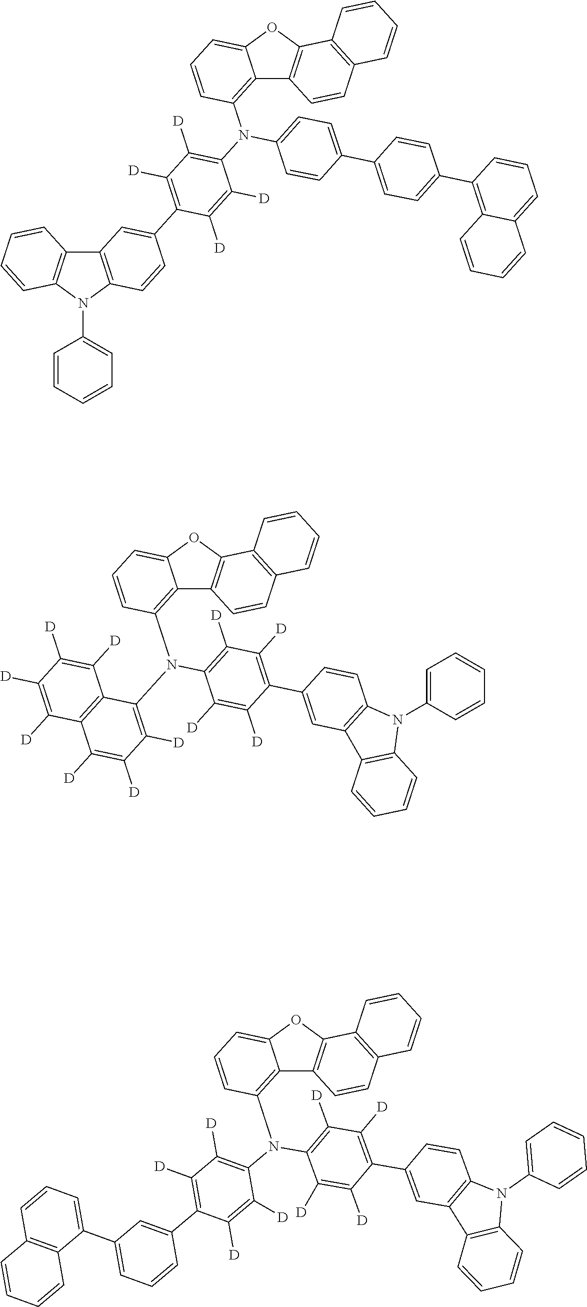 [SMILES: [2H]c1c([2H])c(N(c2ccc(-c3ccc(-c4cccc5ccccc45)cc3)cc2)c2cccc3oc4c5ccccc5ccc4c23)c([2H])c([2H])c1-c1ccc2c(c1)c1ccccc1n2-c1ccccc1 ; [2H]c1c([2H])c(N(c2c([2H])c([2H])c([2H])c3c([2H])c([2H])c([2H])c([2H])c23)c2cccc3oc4c5ccccc5ccc4c23)c([2H])c([2H])c1-c1ccc2c(c1)c1ccccc1n2-c1ccccc1 ; [2H]c1c([2H])c(N(c2c([2H])c([2H])c(-c3ccc4c(c3)c3ccccc3n4-c3ccccc3)c([2H])c2[2H])c2cccc3oc4c5ccccc5ccc4c23)c([2H])c([2H])c1-c1cccc(-c2cccc3ccccc23)c1]

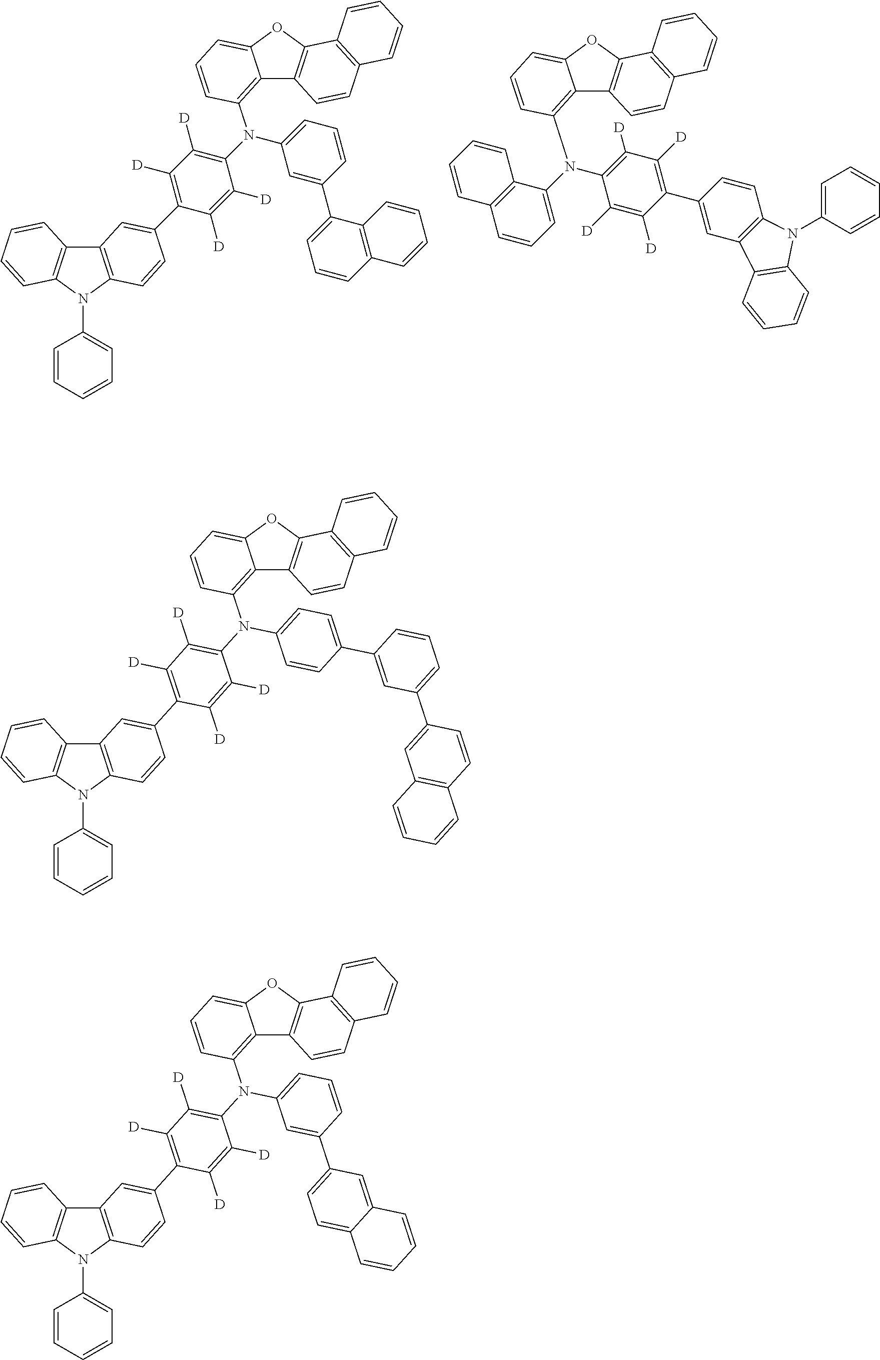

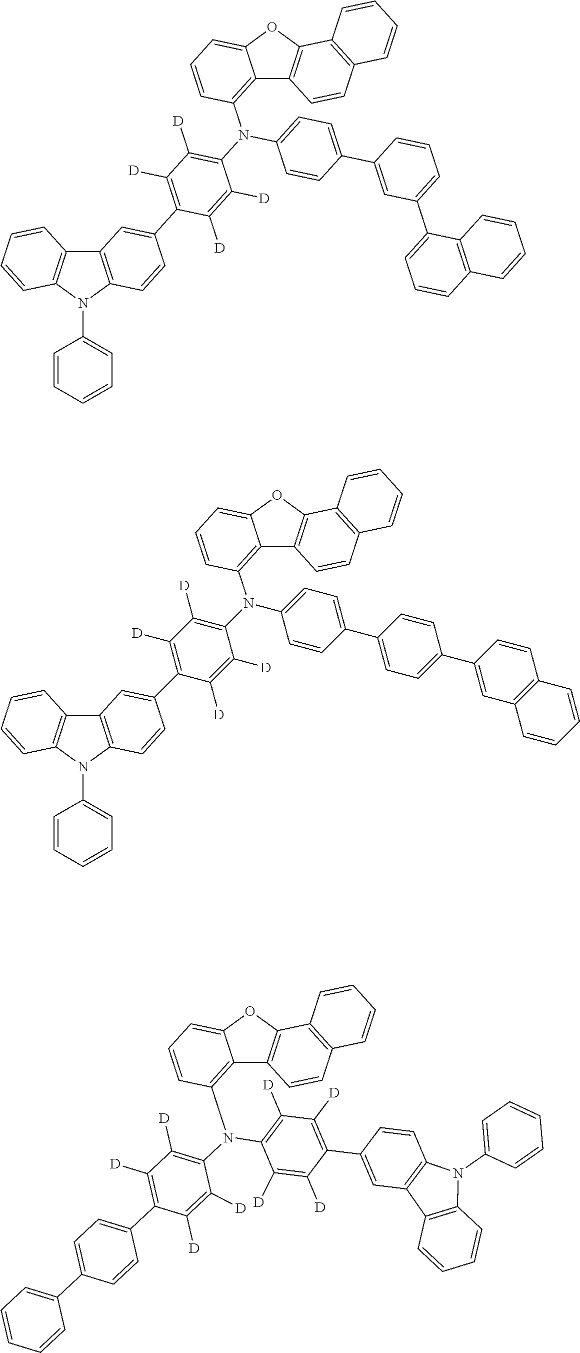 [SMILES: [2H]c1c([2H])c(N(c2ccc(-c3cccc(-c4cccc5ccccc45)c3)cc2)c2cccc3oc4c5ccccc5ccc4c23)c([2H])c([2H])c1-c1ccc2c(c1)c1ccccc1n2-c1ccccc1 ; [2H]c1c([2H])c(N(c2ccc(-c3ccc(-c4ccc5ccccc5c4)cc3)cc2)c2cccc3oc4c5ccccc5ccc4c23)c([2H])c([2H])c1-c1ccc2c(c1)c1ccccc1n2-c1ccccc1 ; [2H]c1c([2H])c(N(c2c([2H])c([2H])c(-c3ccc4c(c3)c3ccccc3n4-c3ccccc3)c([2H])c2[2H])c2cccc3oc4c5ccccc5ccc4c23)c([2H])c([2H])c1-c1ccc(-c2ccccc2)cc1]

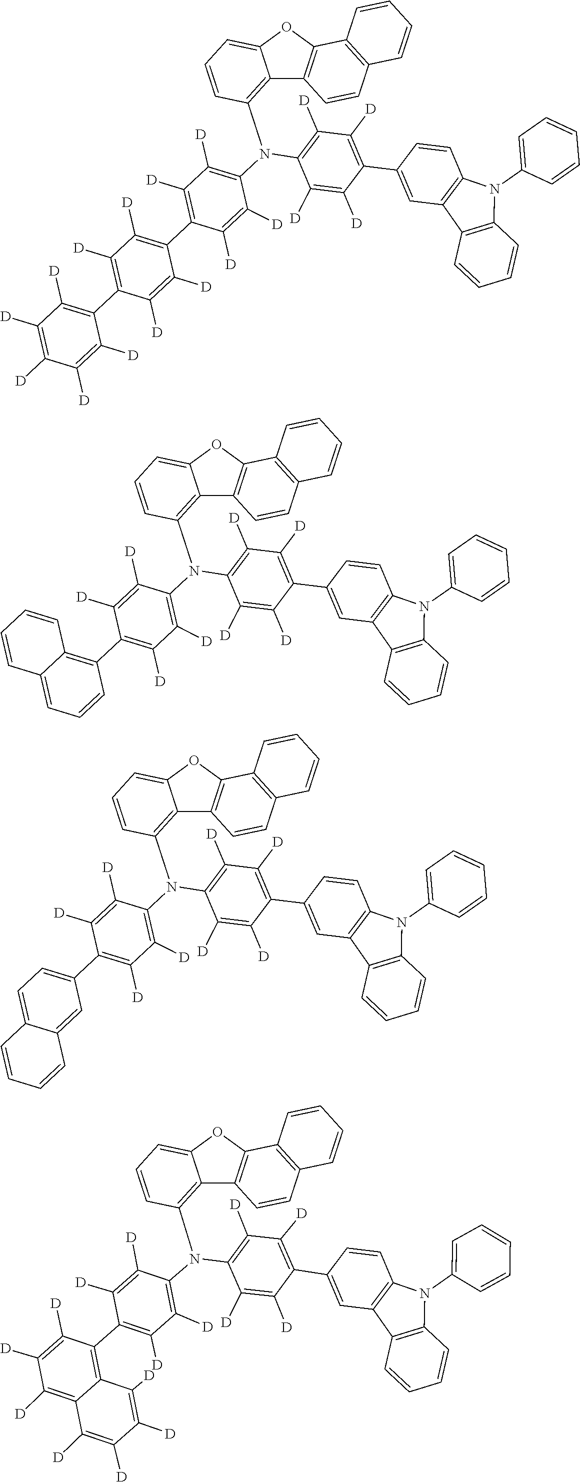

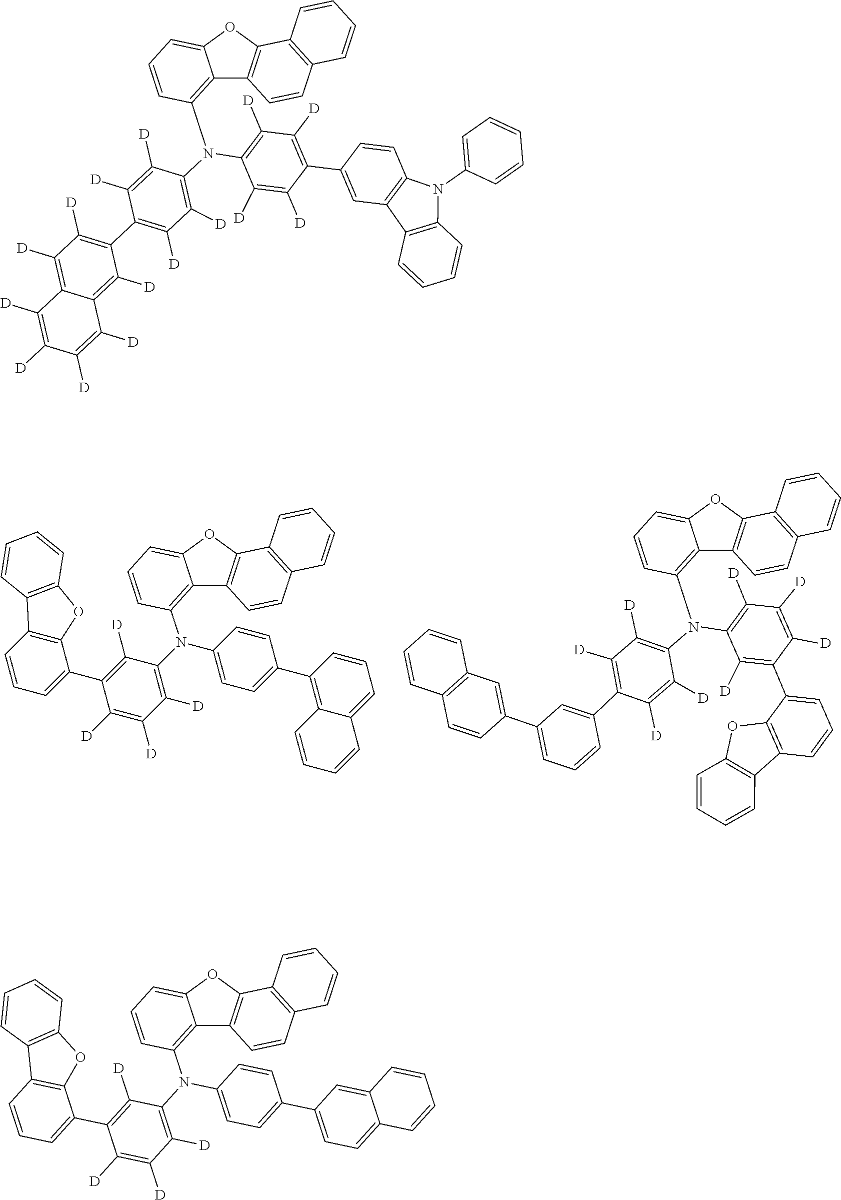

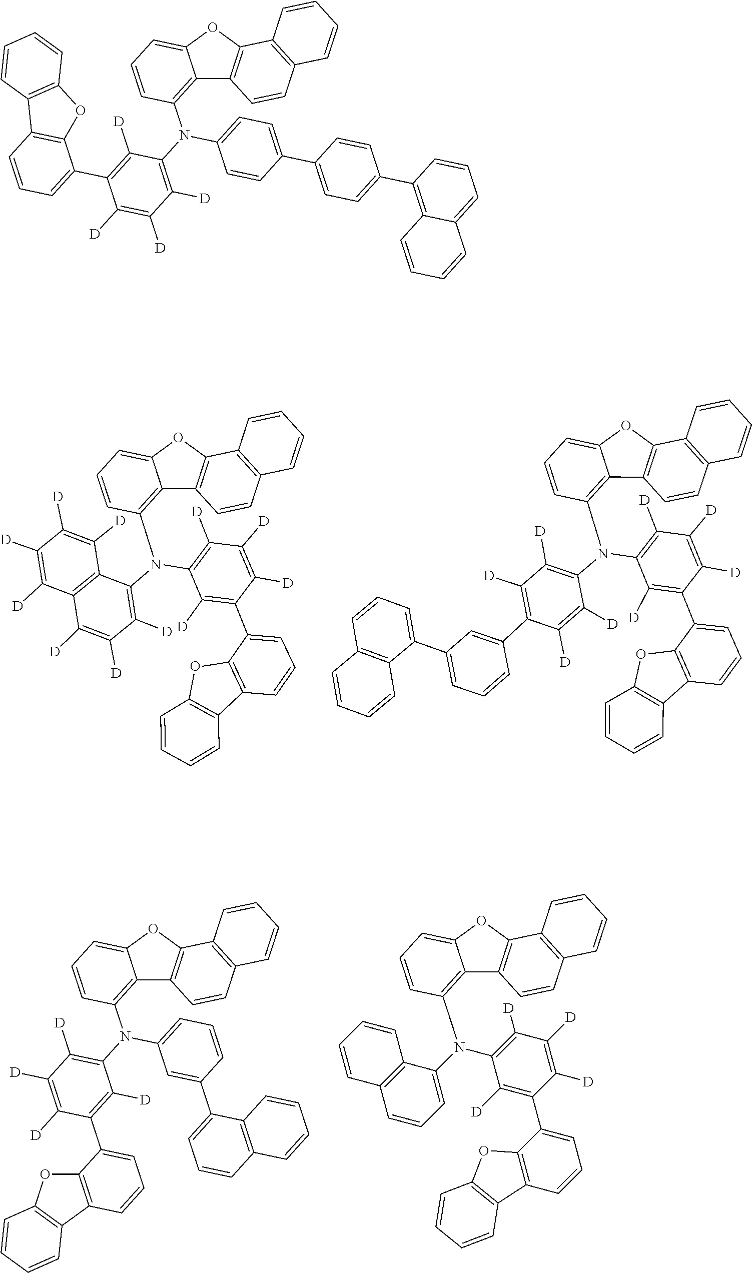

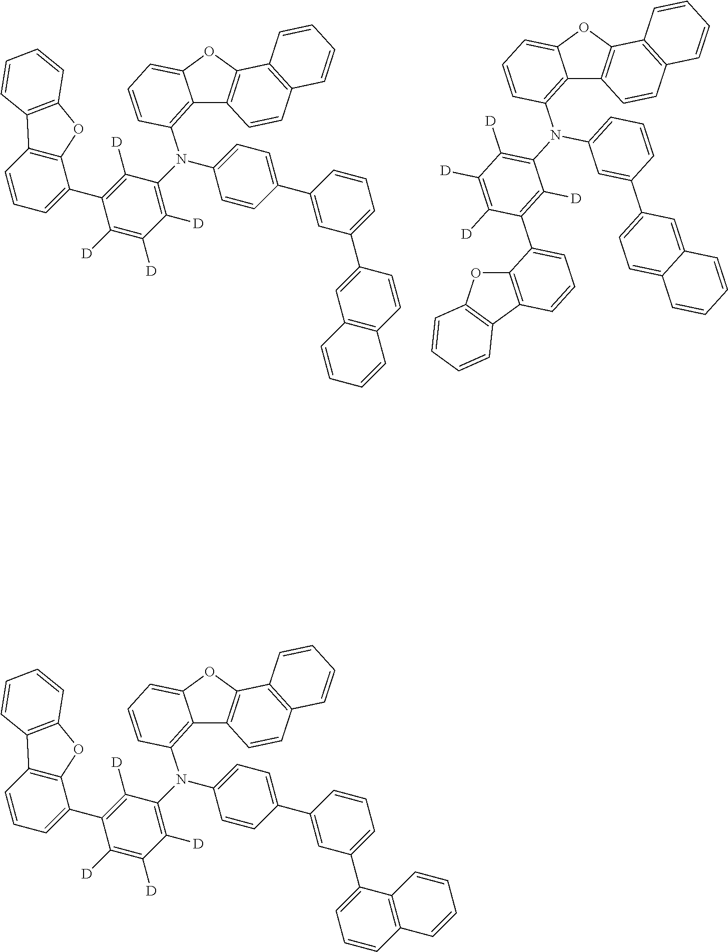

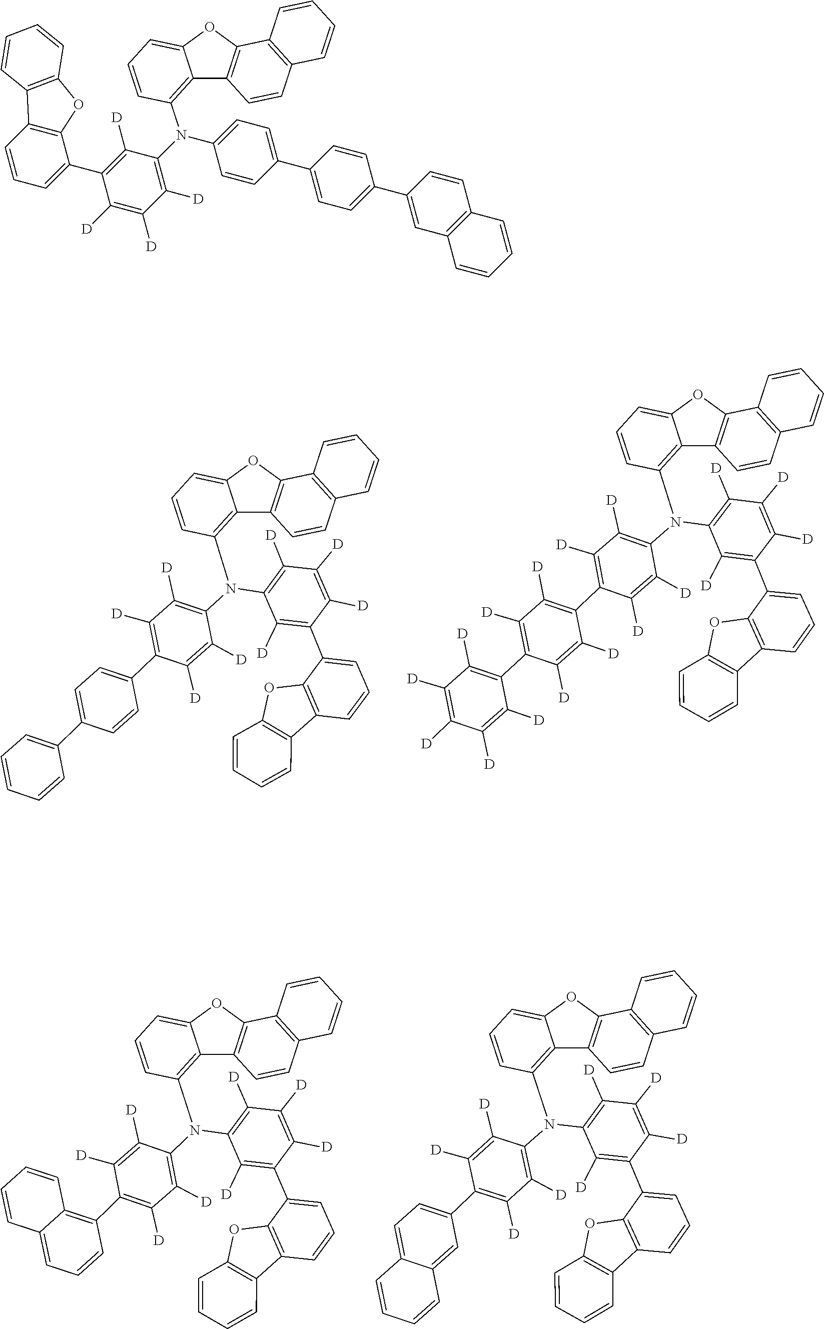

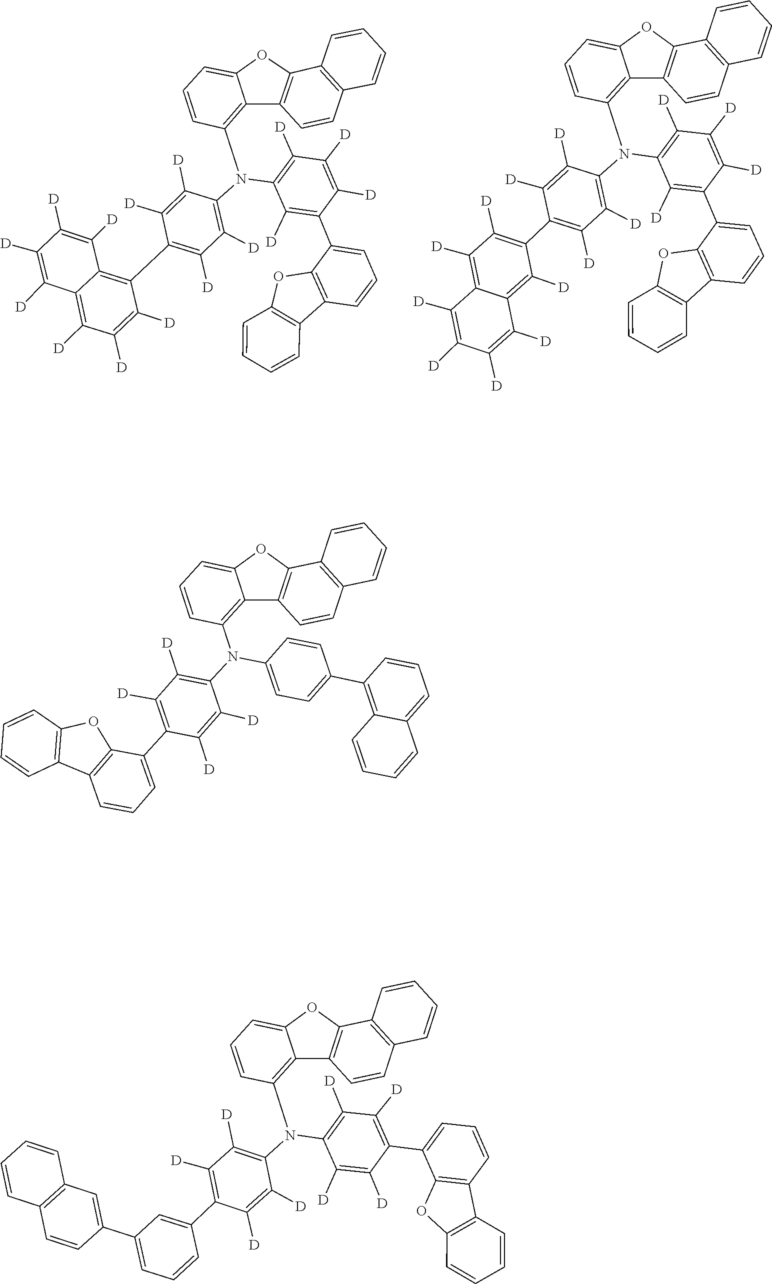

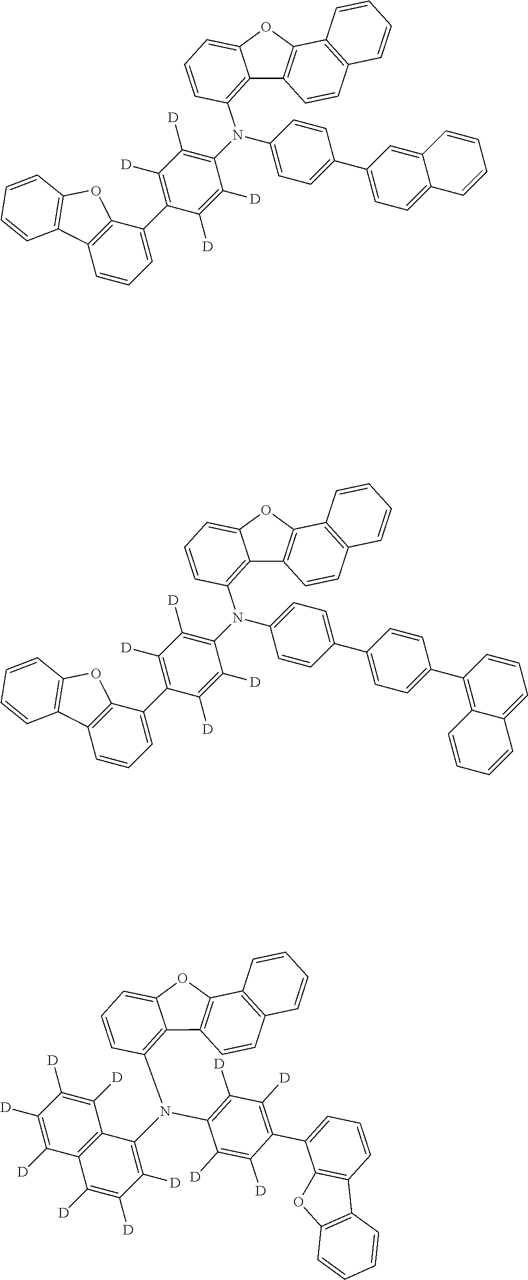 [SMILES: [2H]c1c([2H])c(N(c2ccc(-c3ccc4ccccc4c3)cc2)c2cccc3oc4c5ccccc5ccc4c23)c([2H])c([2H])c1-c1cccc2c1oc1ccccc12 ; [2H]c1c([2H])c(N(c2ccc(-c3ccc(-c4cccc5ccccc45)cc3)cc2)c2cccc3oc4c5ccccc5ccc4c23)c([2H])c([2H])c1-c1cccc2c1oc1ccccc12 ; [2H]c1c([2H])c(N(c2c([2H])c([2H])c([2H])c3c([2H])c([2H])c([2H])c([2H])c23)c2cccc3oc4c5ccccc5ccc4c23)c([2H])c([2H])c1-c1cccc2c1oc1ccccc12]

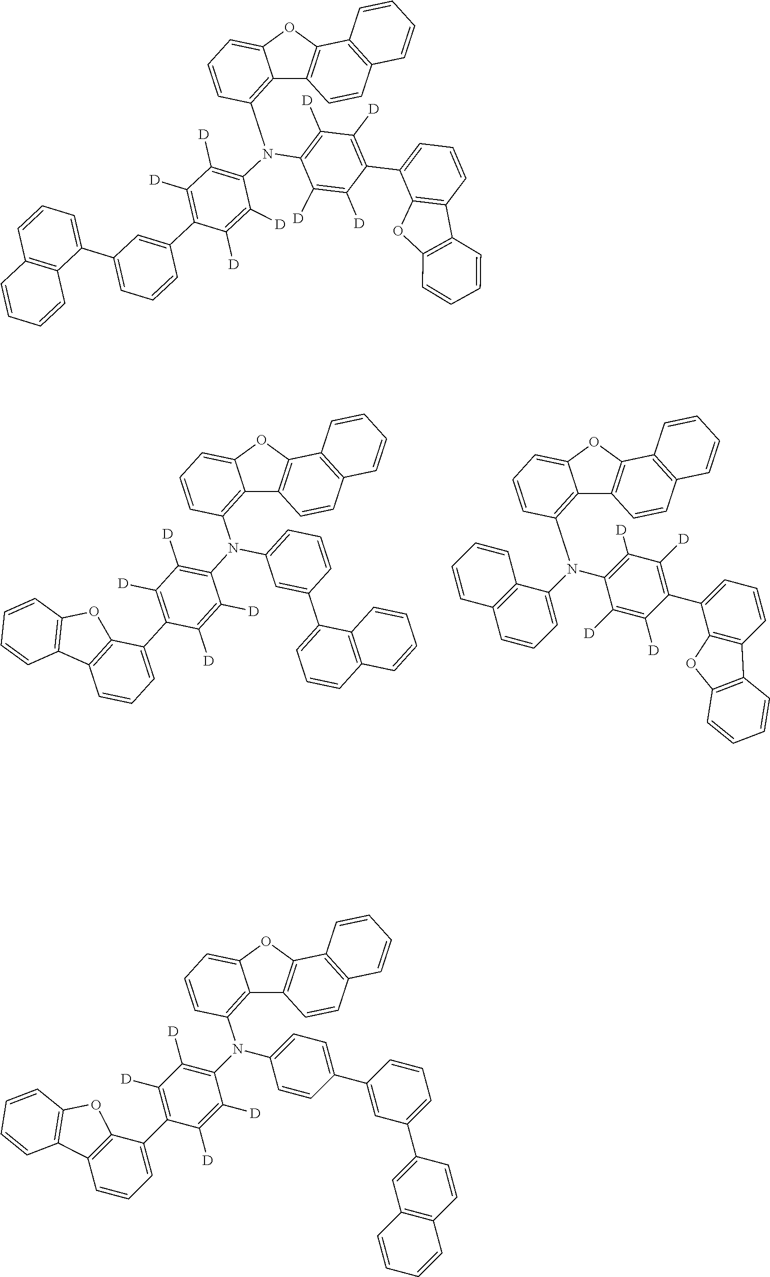 [SMILES: [2H]c1c([2H])c(N(c2c([2H])c([2H])c(-c3cccc4c3oc3ccccc34)c([2H])c2[2H])c2cccc3oc4c5ccccc5ccc4c23)c([2H])c([2H])c1-c1cccc(-c2cccc3ccccc23)c1 ; [2H]c1c([2H])c(N(c2cccc(-c3cccc4ccccc34)c2)c2cccc3oc4c5ccccc5ccc4c23)c([2H])c([2H])c1-c1cccc2c1oc1ccccc12 ; [2H]c1c([2H])c(N(c2cccc3ccccc23)c2cccc3oc4c5ccccc5ccc4c23)c([2H])c([2H])c1-c1cccc2c1oc1ccccc12 ; [2H]c1c([2H])c(N(c2ccc(-c3cccc(-c4ccc5ccccc5c4)c3)cc2)c2cccc3oc4c5ccccc5ccc4c23)c([2H])c([2H])c1-c1cccc2c1oc1ccccc12]

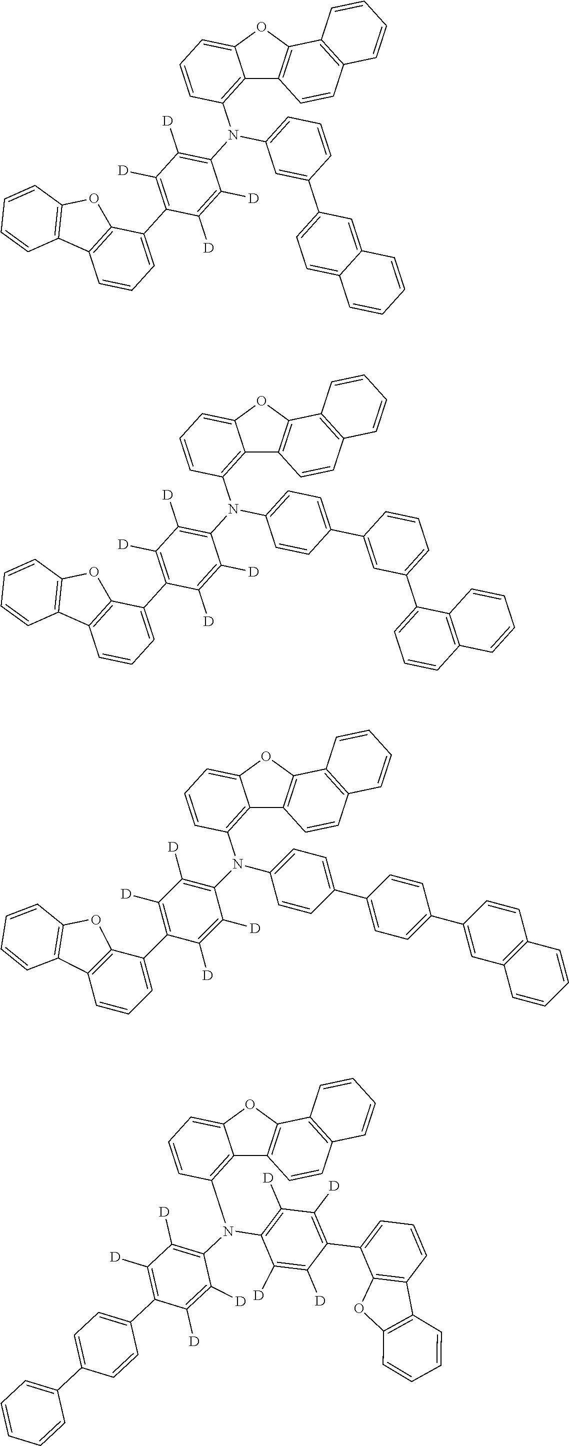 [SMILES: [2H]c1c([2H])c(N(c2cccc(-c3ccc4ccccc4c3)c2)c2cccc3oc4c5ccccc5ccc4c23)c([2H])c([2H])c1-c1cccc2c1oc1ccccc12 ; [2H]c1c([2H])c(N(c2ccc(-c3cccc(-c4cccc5ccccc45)c3)cc2)c2cccc3oc4c5ccccc5ccc4c23)c([2H])c([2H])c1-c1cccc2c1oc1ccccc12 ; [2H]c1c([2H])c(N(c2ccc(-c3ccc(-c4ccc5ccccc5c4)cc3)cc2)c2cccc3oc4c5ccccc5ccc4c23)c([2H])c([2H])c1-c1cccc2c1oc1ccccc12 ; [2H]c1c([2H])c(N(c2c([2H])c([2H])c(-c3cccc4c3oc3ccccc34)c([2H])c2[2H])c2cccc3oc4c5ccccc5ccc4c23)c([2H])c([2H])c1-c1ccc(-c2ccccc2)cc1]

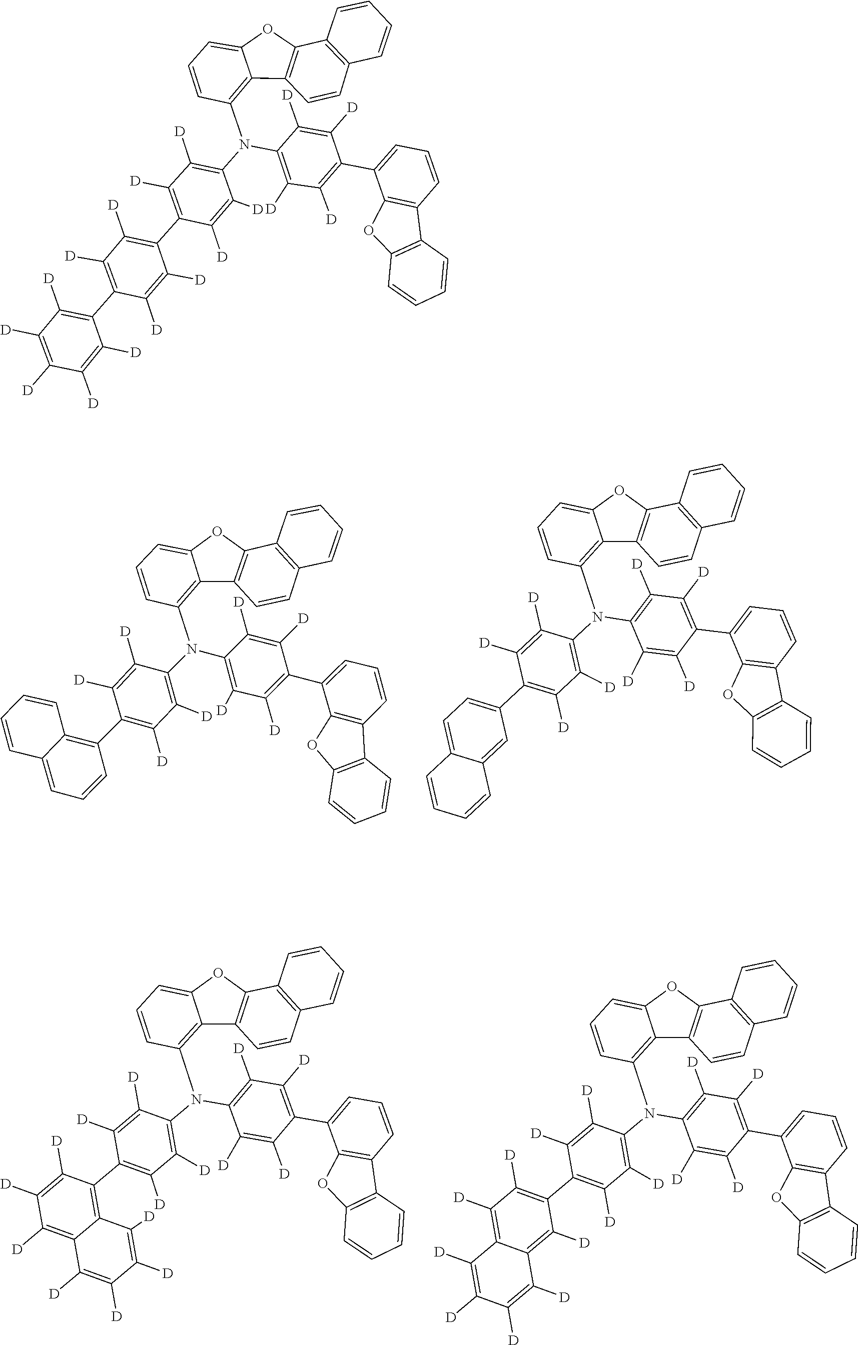

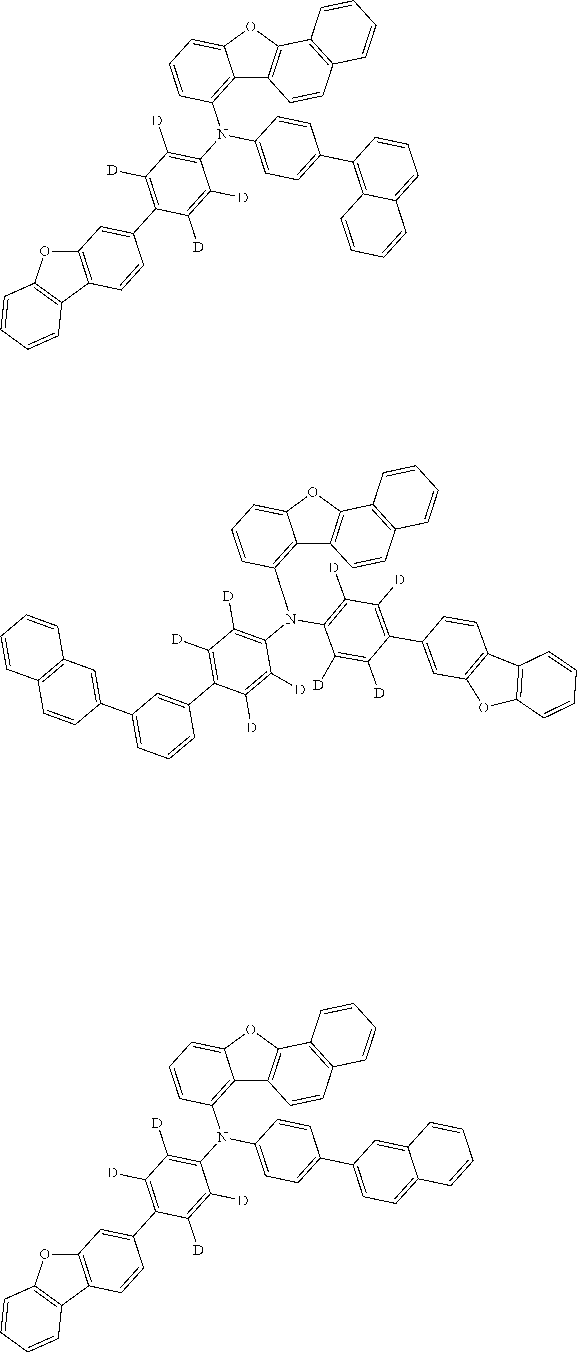 [SMILES: [2H]c1c([2H])c(N(c2ccc(-c3cccc4ccccc34)cc2)c2cccc3oc4c5ccccc5ccc4c23)c([2H])c([2H])c1-c1ccc2c(c1)oc1ccccc12 ; [2H]c1c([2H])c(N(c2c([2H])c([2H])c(-c3ccc4c(c3)oc3ccccc34)c([2H])c2[2H])c2cccc3oc4c5ccccc5ccc4c23)c([2H])c([2H])c1-c1cccc(-c2ccc3ccccc3c2)c1 ; [2H]c1c([2H])c(N(c2ccc(-c3ccc4ccccc4c3)cc2)c2cccc3oc4c5ccccc5ccc4c23)c([2H])c([2H])c1-c1ccc2c(c1)oc1ccccc12]

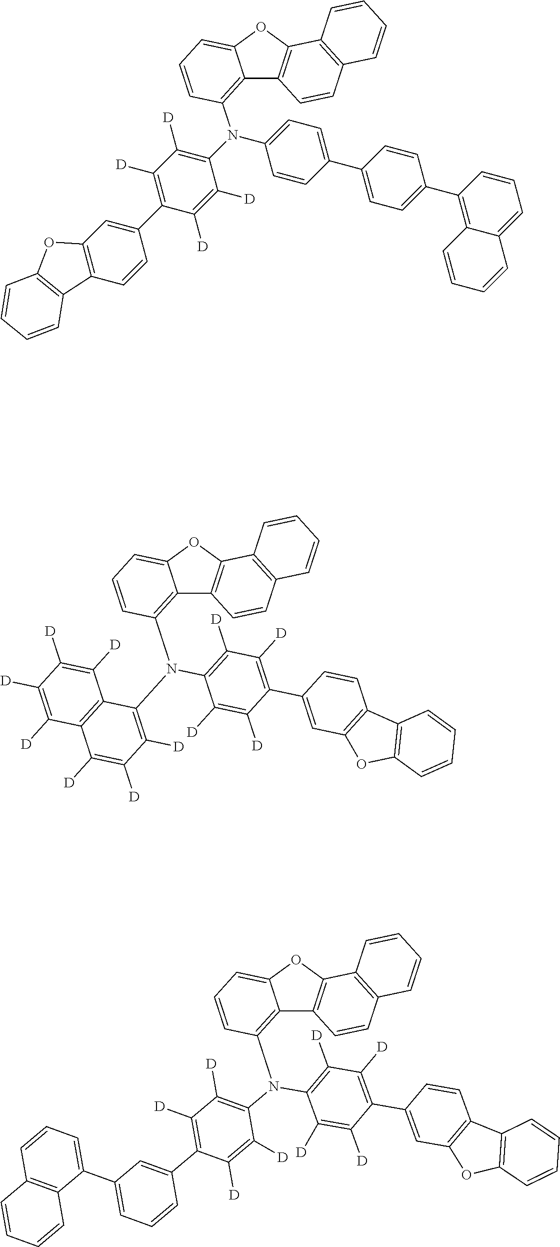 [SMILES: [2H]c1c([2H])c(N(c2ccc(-c3ccc(-c4cccc5ccccc45)cc3)cc2)c2cccc3oc4c5ccccc5ccc4c23)c([2H])c([2H])c1-c1ccc2c(c1)oc1ccccc12 ; [2H]c1c([2H])c(N(c2c([2H])c([2H])c([2H])c3c([2H])c([2H])c([2H])c([2H])c23)c2cccc3oc4c5ccccc5ccc4c23)c([2H])c([2H])c1-c1ccc2c(c1)oc1ccccc12 ; [2H]c1c([2H])c(N(c2c([2H])c([2H])c(-c3ccc4c(c3)oc3ccccc34)c([2H])c2[2H])c2cccc3oc4c5ccccc5ccc4c23)c([2H])c([2H])c1-c1cccc(-c2cccc3ccccc23)c1]

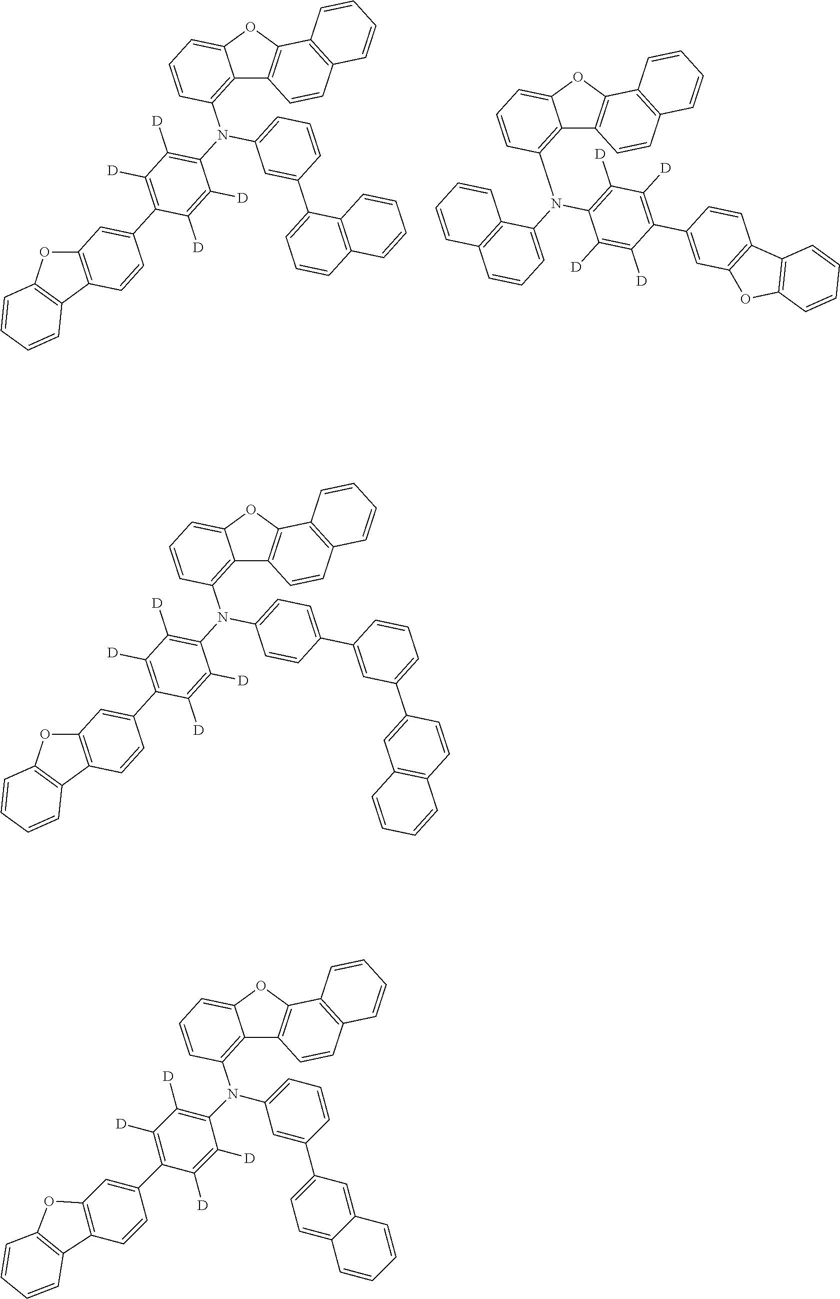

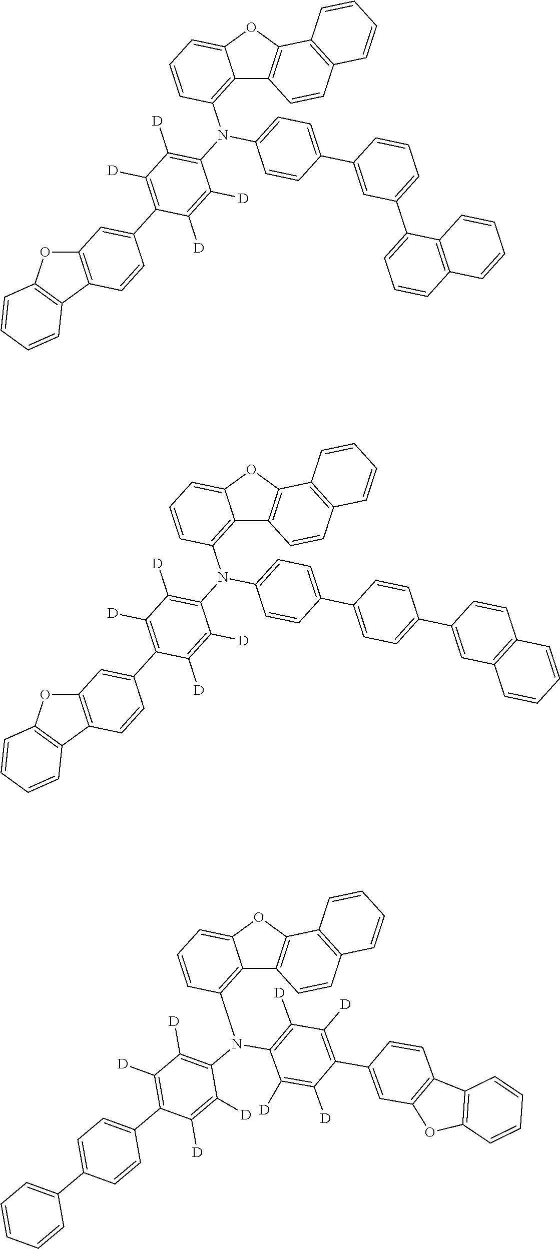 [SMILES: [2H]c1c([2H])c(N(c2ccc(-c3cccc(-c4cccc5ccccc45)c3)cc2)c2cccc3oc4c5ccccc5ccc4c23)c([2H])c([2H])c1-c1ccc2c(c1)oc1ccccc12 ; [2H]c1c([2H])c(N(c2ccc(-c3ccc(-c4ccc5ccccc5c4)cc3)cc2)c2cccc3oc4c5ccccc5ccc4c23)c([2H])c([2H])c1-c1ccc2c(c1)oc1ccccc12 ; [2H]c1c([2H])c(N(c2c([2H])c([2H])c(-c3ccc4c(c3)oc3ccccc34)c([2H])c2[2H])c2cccc3oc4c5ccccc5ccc4c23)c([2H])c([2H])c1-c1ccc(-c2ccccc2)cc1]

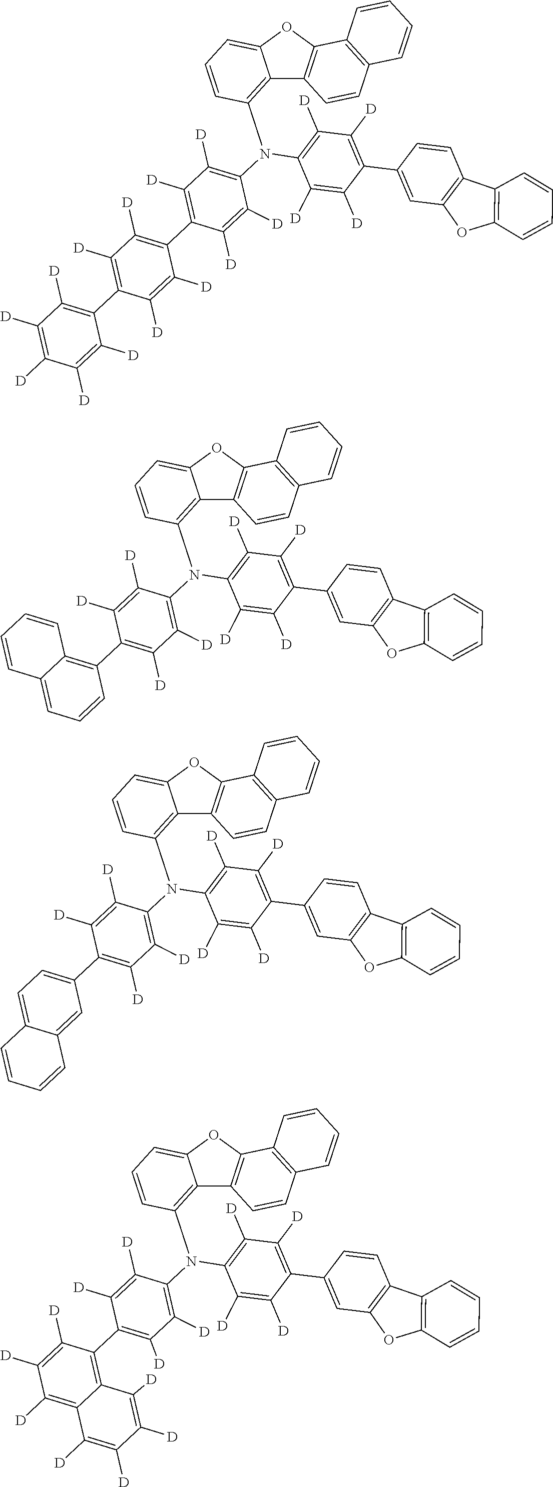

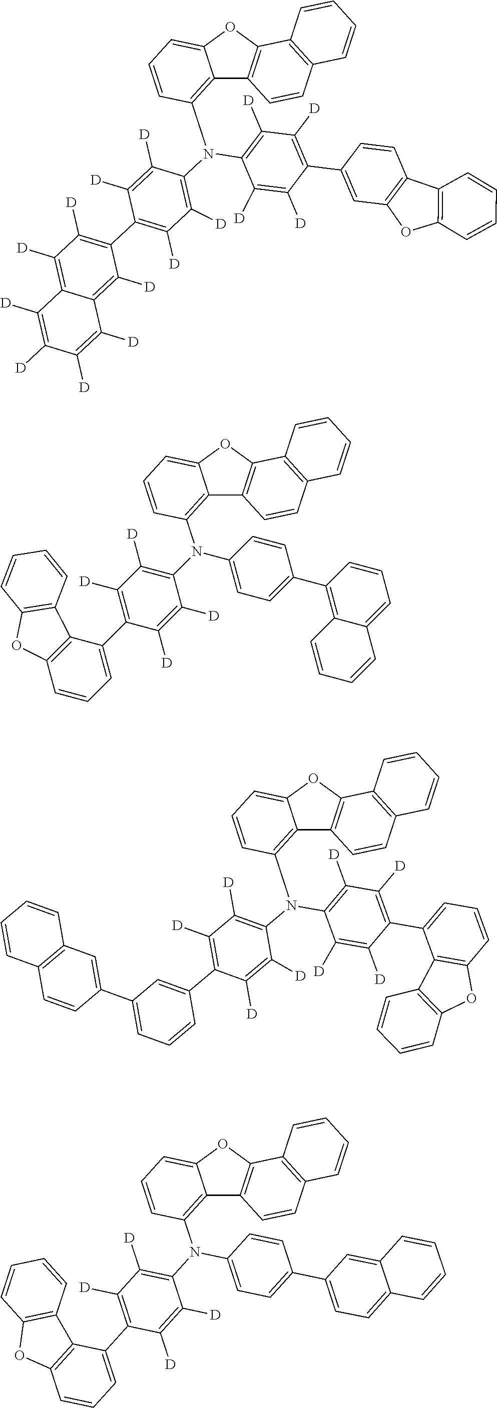

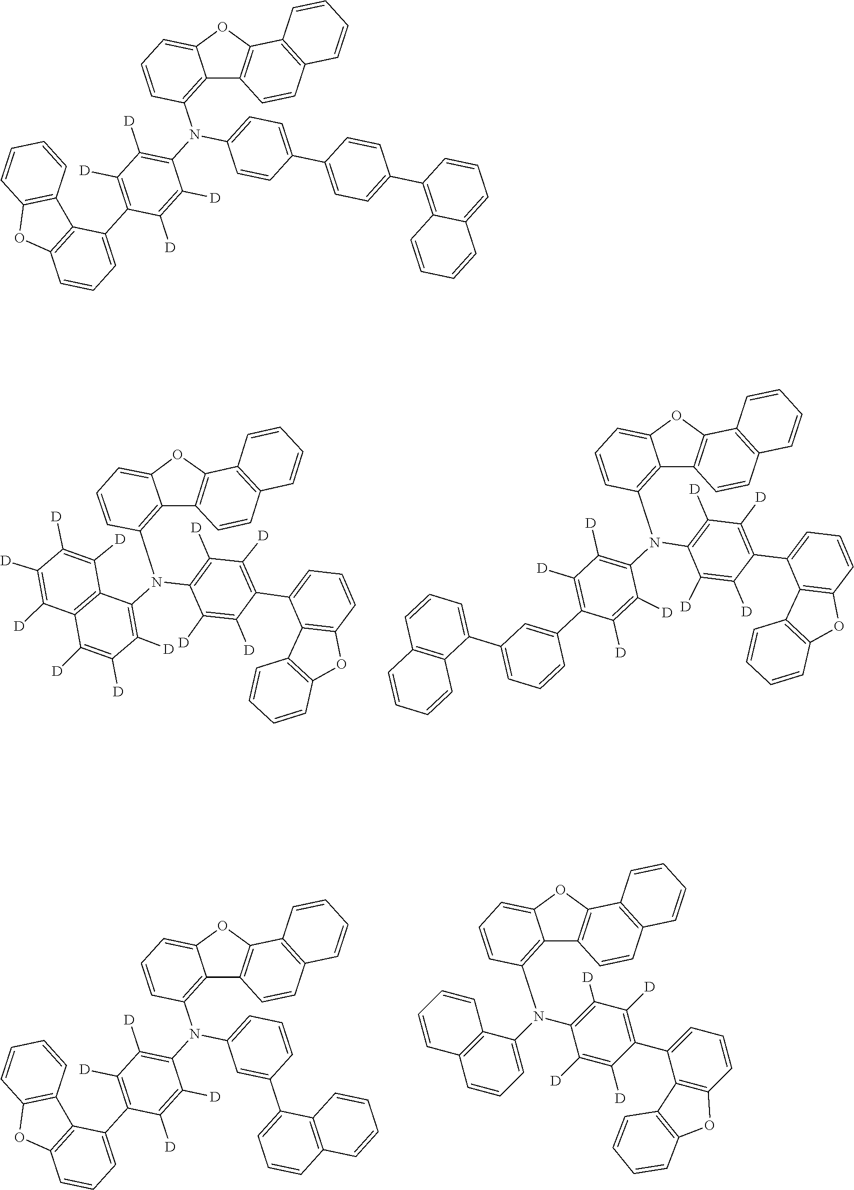

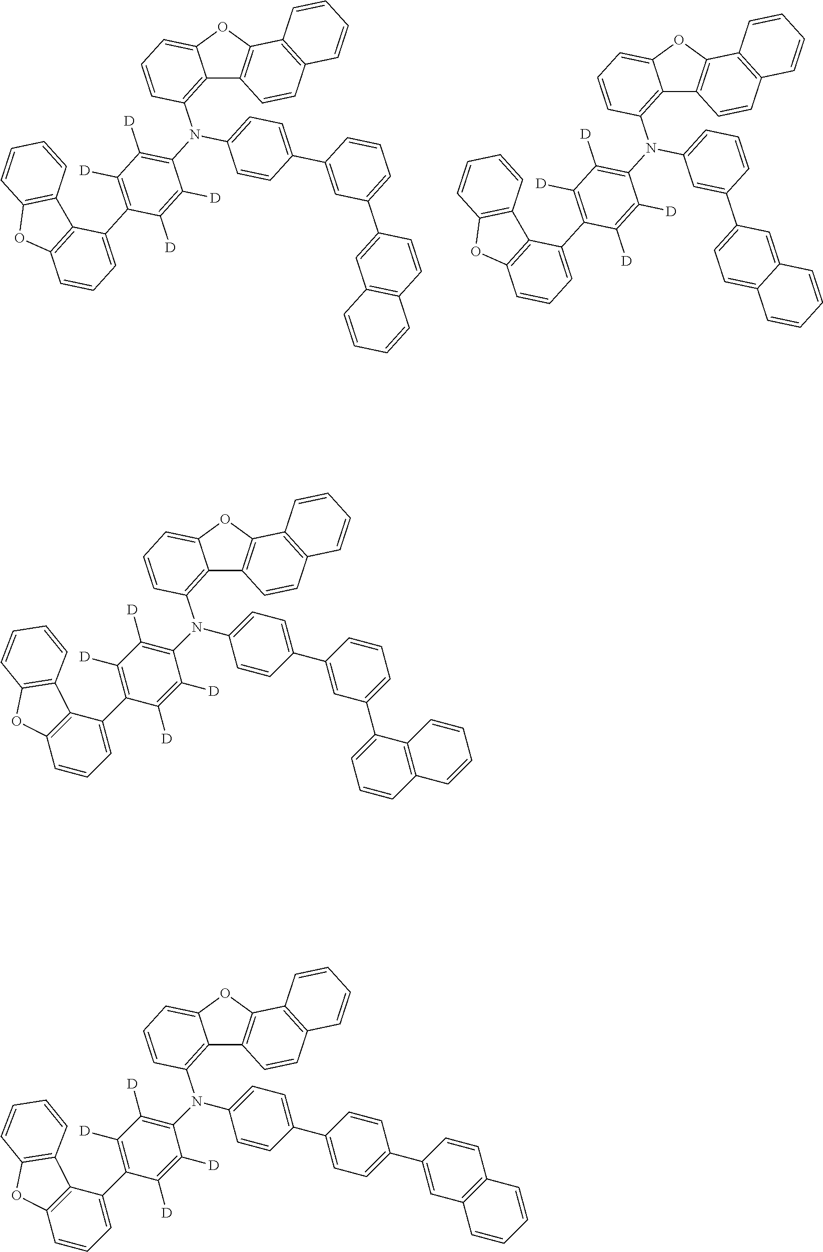 [SMILES: [2H]c1c([2H])c(N(c2ccc(-c3cccc(-c4ccc5ccccc5c4)c3)cc2)c2cccc3oc4c5ccccc5ccc4c23)c([2H])c([2H])c1-c1cccc2oc3ccccc3c12 ; [2H]c1c([2H])c(N(c2cccc(-c3ccc4ccccc4c3)c2)c2cccc3oc4c5ccccc5ccc4c23)c([2H])c([2H])c1-c1cccc2oc3ccccc3c12 ; [2H]c1c([2H])c(N(c2ccc(-c3cccc(-c4cccc5ccccc45)c3)cc2)c2cccc3oc4c5ccccc5ccc4c23)c([2H])c([2H])c1-c1cccc2oc3ccccc3c12 ; [2H]c1c([2H])c(N(c2ccc(-c3ccc(-c4ccc5ccccc5c4)cc3)cc2)c2cccc3oc4c5ccccc5ccc4c23)c([2H])c([2H])c1-c1cccc2oc3ccccc3c12]

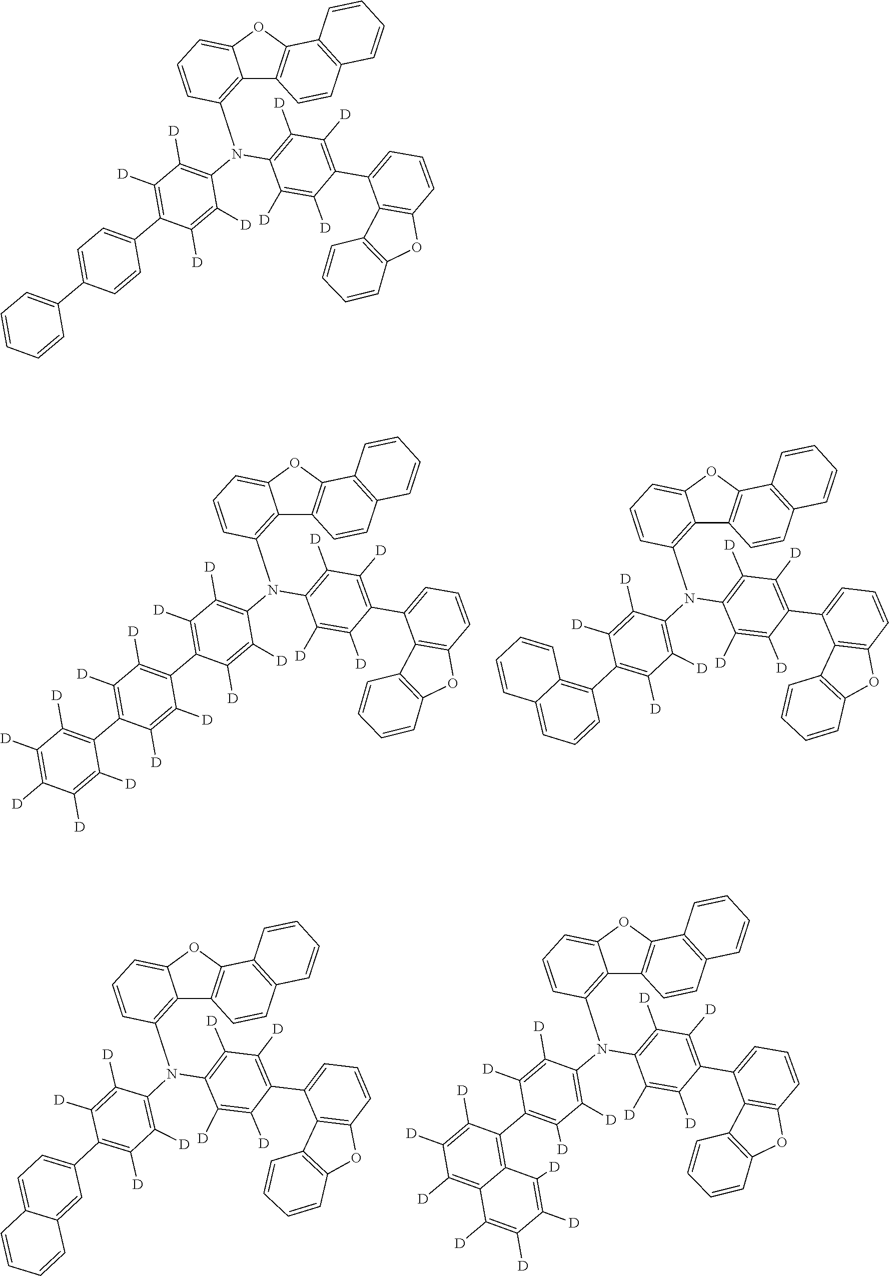

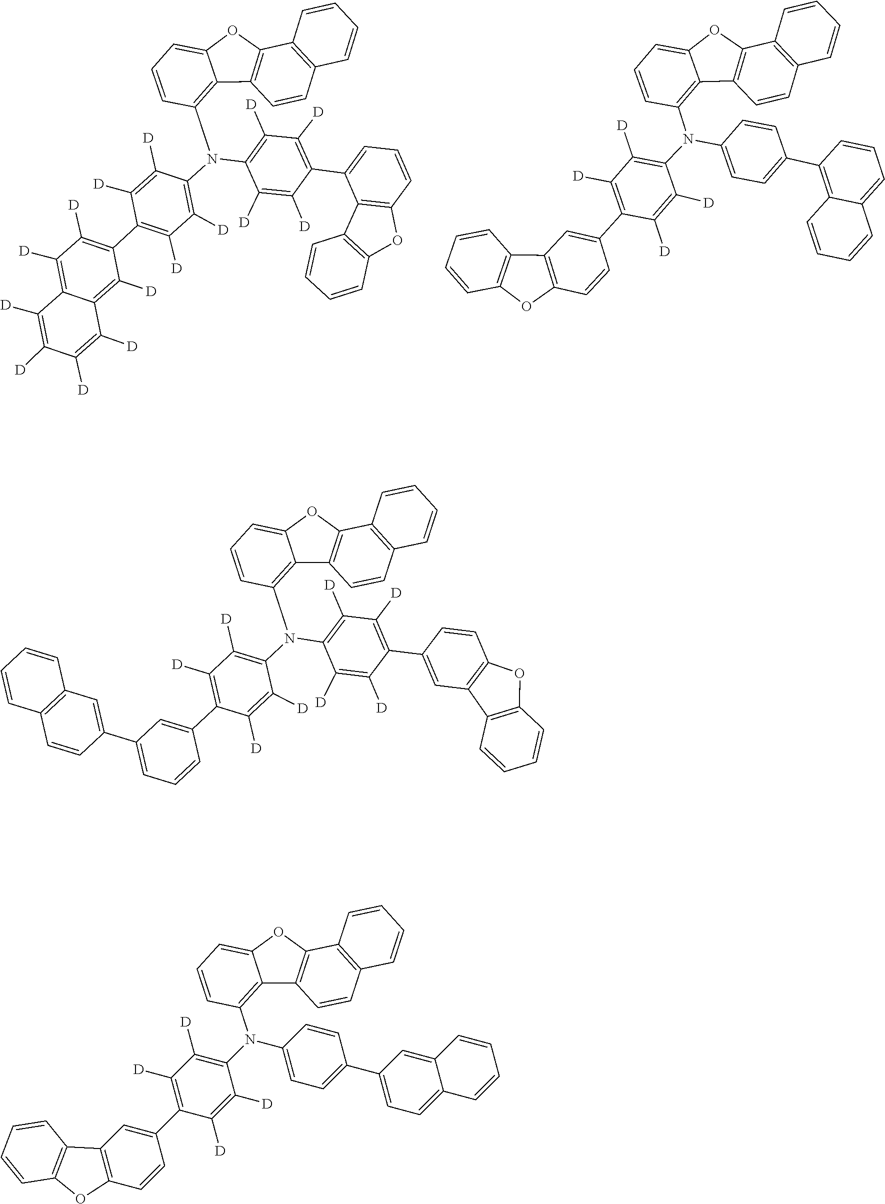

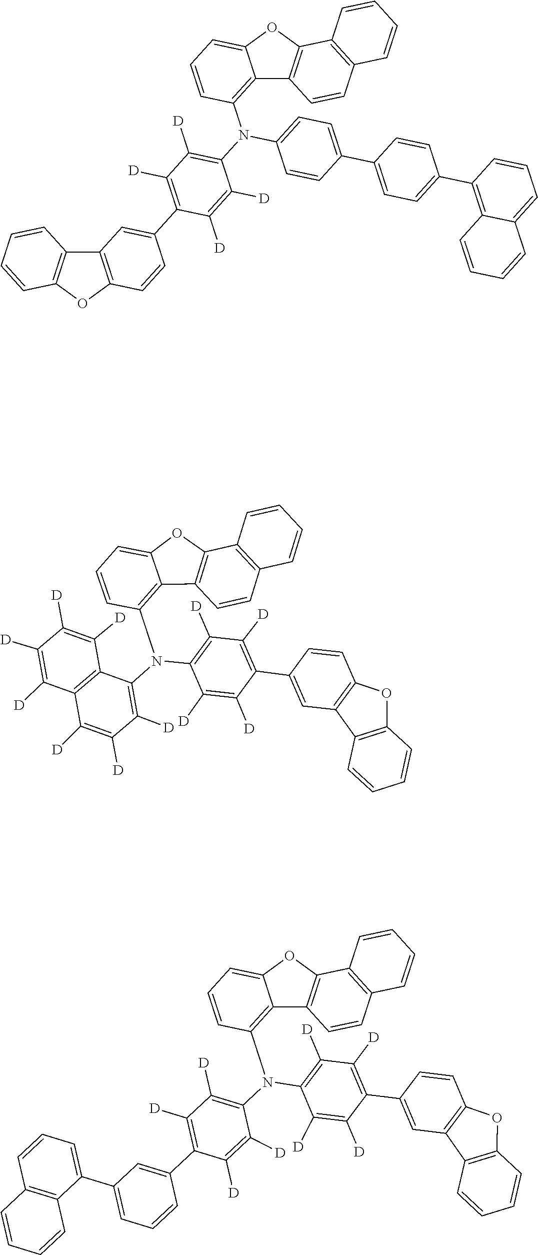 [SMILES: [2H]c1c([2H])c(N(c2ccc(-c3ccc(-c4cccc5ccccc45)cc3)cc2)c2cccc3oc4c5ccccc5ccc4c23)c([2H])c([2H])c1-c1ccc2oc3ccccc3c2c1 ; [2H]c1c([2H])c(N(c2c([2H])c([2H])c([2H])c3c([2H])c([2H])c([2H])c([2H])c23)c2cccc3oc4c5ccccc5ccc4c23)c([2H])c([2H])c1-c1ccc2oc3ccccc3c2c1 ; [2H]c1c([2H])c(N(c2c([2H])c([2H])c(-c3ccc4oc5ccccc5c4c3)c([2H])c2[2H])c2cccc3oc4c5ccccc5ccc4c23)c([2H])c([2H])c1-c1cccc(-c2cccc3ccccc23)c1]

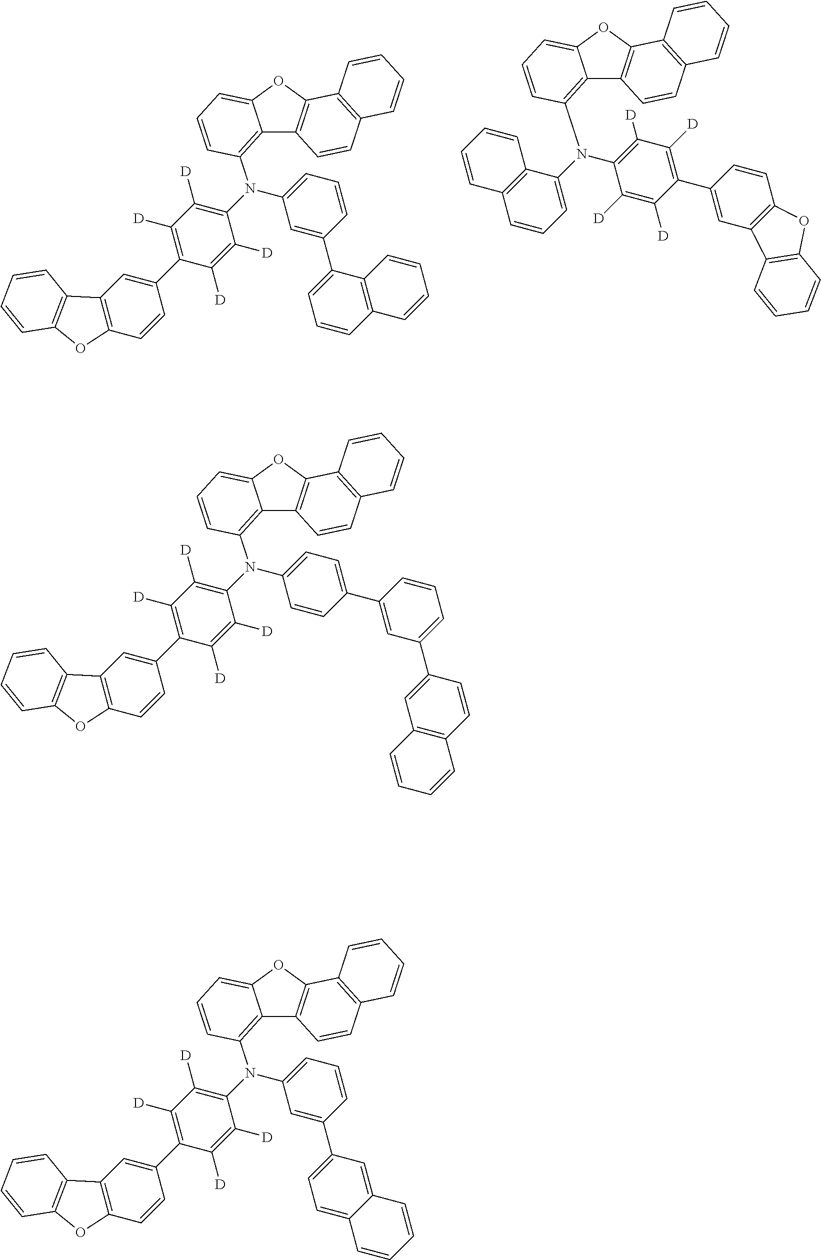 [SMILES: [2H]c1c([2H])c(N(c2cccc(-c3cccc4ccccc34)c2)c2cccc3oc4c5ccccc5ccc4c23)c([2H])c([2H])c1-c1ccc2oc3ccccc3c2c1 ; [2H]c1c([2H])c(N(c2cccc3ccccc23)c2cccc3oc4c5ccccc5ccc4c23)c([2H])c([2H])c1-c1ccc2oc3ccccc3c2c1 ; [2H]c1c([2H])c(N(c2ccc(-c3cccc(-c4ccc5ccccc5c4)c3)cc2)c2cccc3oc4c5ccccc5ccc4c23)c([2H])c([2H])c1-c1ccc2oc3ccccc3c2c1 ; [2H]c1c([2H])c(N(c2cccc(-c3ccc4ccccc4c3)c2)c2cccc3oc4c5ccccc5ccc4c23)c([2H])c([2H])c1-c1ccc2oc3ccccc3c2c1]

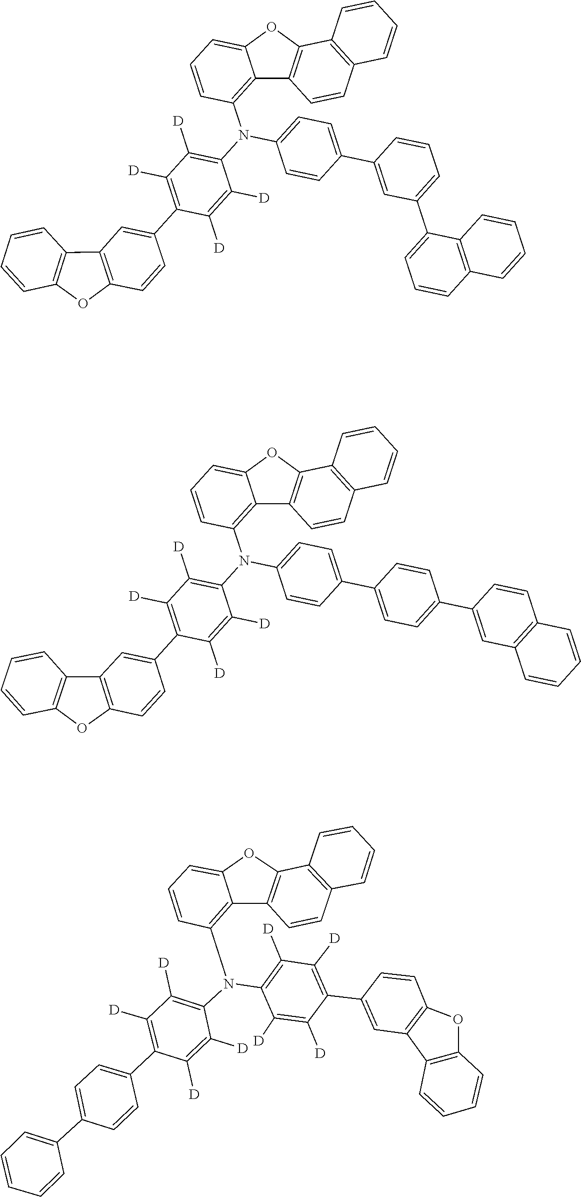 [SMILES: [2H]c1c([2H])c(N(c2ccc(-c3cccc(-c4cccc5ccccc45)c3)cc2)c2cccc3oc4c5ccccc5ccc4c23)c([2H])c([2H])c1-c1ccc2oc3ccccc3c2c1 ; [2H]c1c([2H])c(N(c2ccc(-c3ccc(-c4ccc5ccccc5c4)cc3)cc2)c2cccc3oc4c5ccccc5ccc4c23)c([2H])c([2H])c1-c1ccc2oc3ccccc3c2c1 ; [2H]c1c([2H])c(N(c2c([2H])c([2H])c(-c3ccc4oc5ccccc5c4c3)c([2H])c2[2H])c2cccc3oc4c5ccccc5ccc4c23)c([2H])c([2H])c1-c1ccc(-c2ccccc2)cc1]

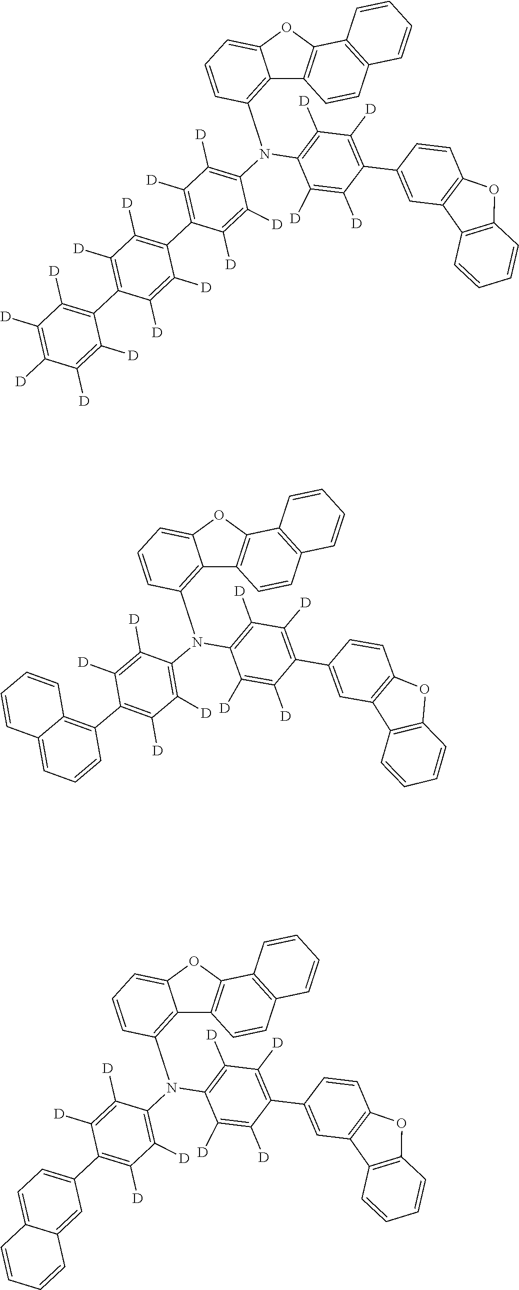 [SMILES: [2H]c1c([2H])c([2H])c(-c2c([2H])c([2H])c(-c3c([2H])c([2H])c(N(c4c([2H])c([2H])c(-c5ccc6oc7ccccc7c6c5)c([2H])c4[2H])c4cccc5oc6c7ccccc7ccc6c45)c([2H])c3[2H])c([2H])c2[2H])c([2H])c1[2H] ; [2H]c1c([2H])c(N(c2c([2H])c([2H])c(-c3cccc4ccccc34)c([2H])c2[2H])c2cccc3oc4c5ccccc5ccc4c23)c([2H])c([2H])c1-c1ccc2oc3ccccc3c2c1 ; [2H]c1c([2H])c(N(c2c([2H])c([2H])c(-c3ccc4oc5ccccc5c4c3)c([2H])c2[2H])c2cccc3oc4c5ccccc5ccc4c23)c([2H])c([2H])c1-c1ccc2ccccc2c1]

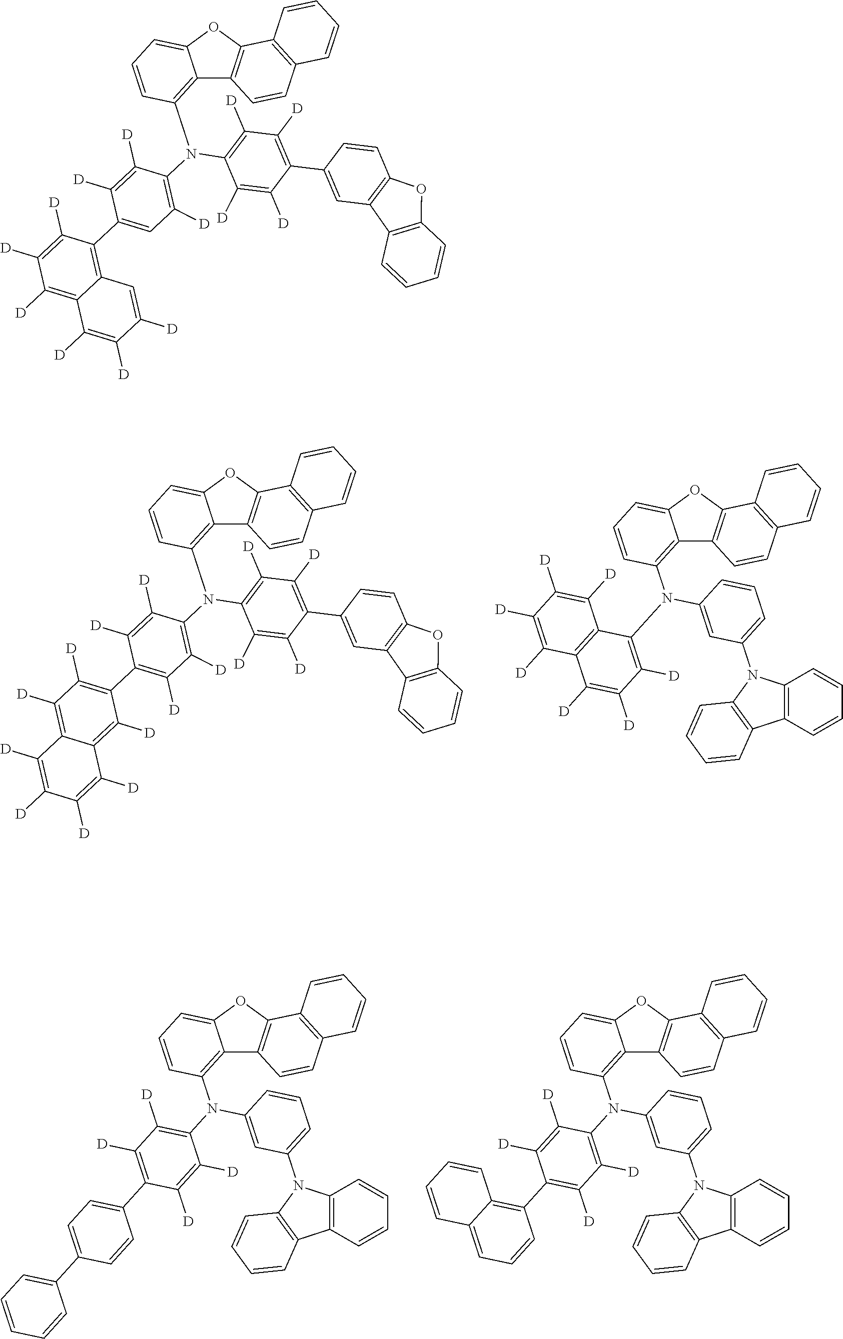

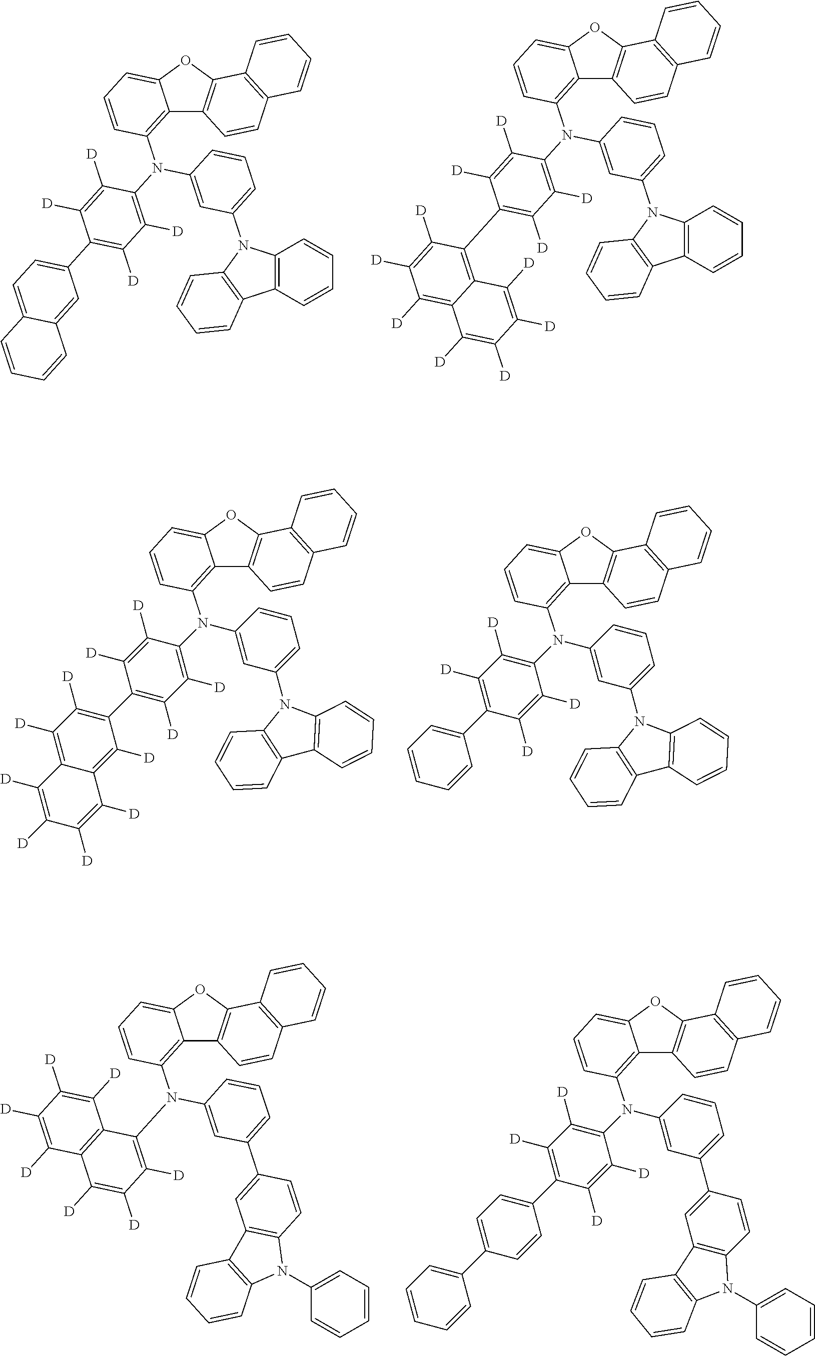

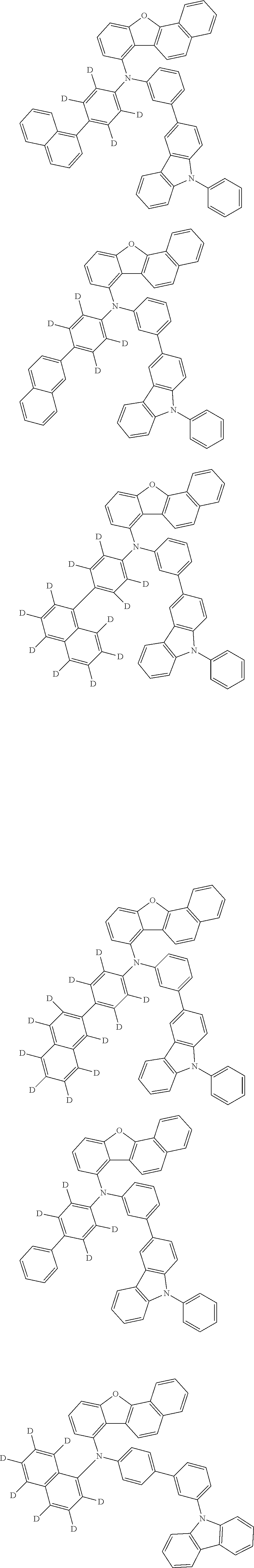

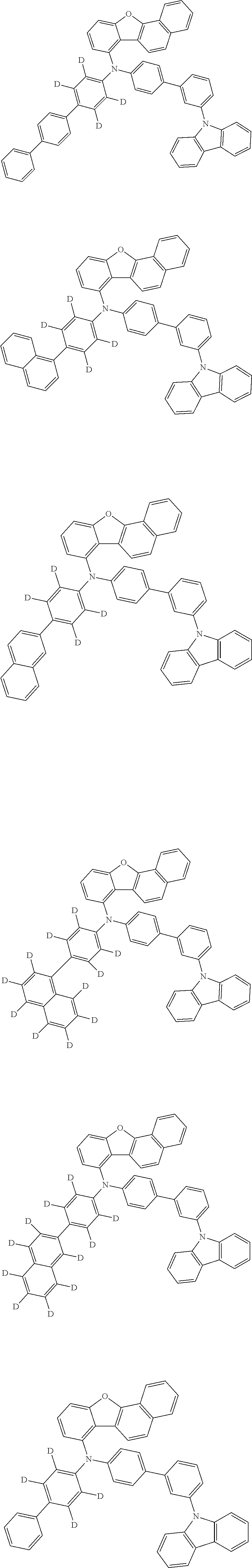

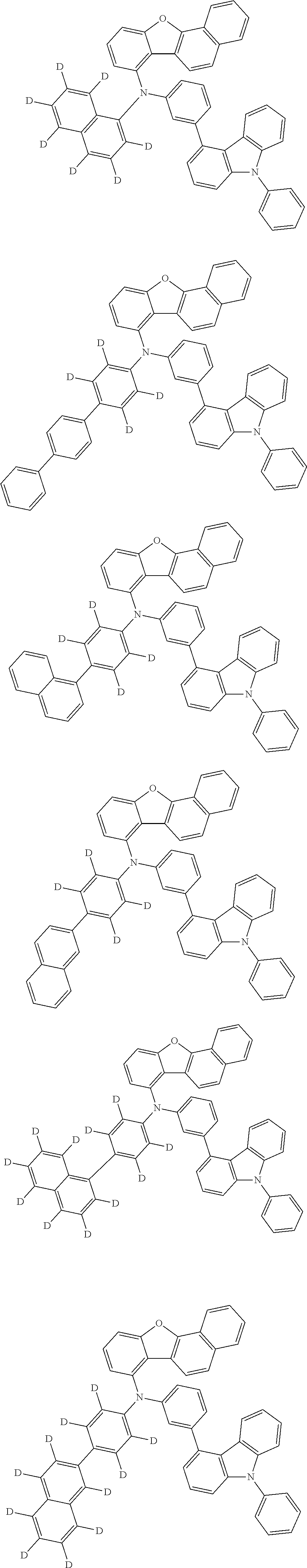

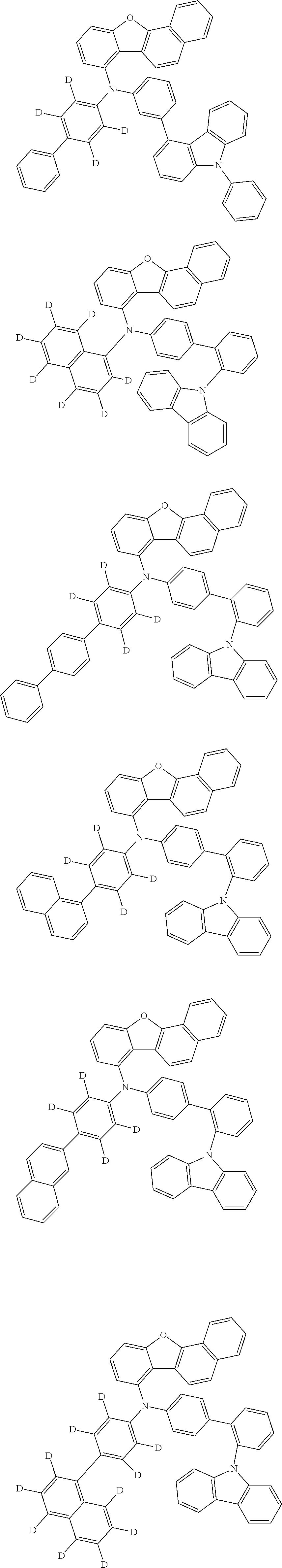

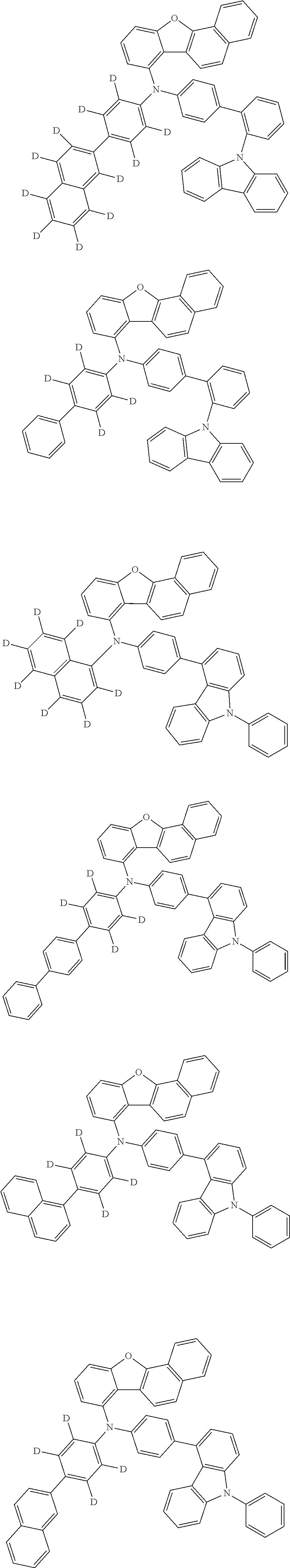

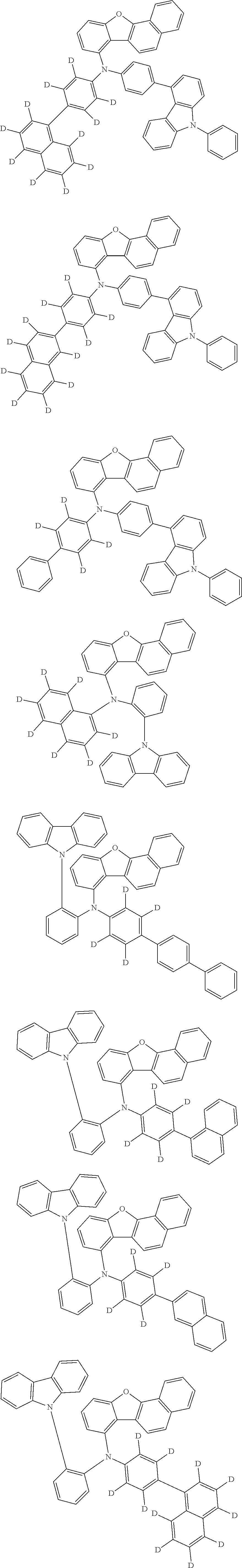

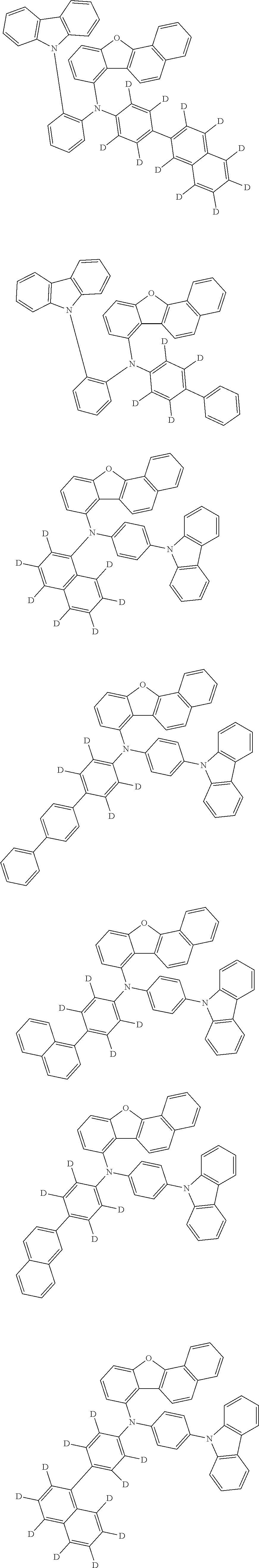

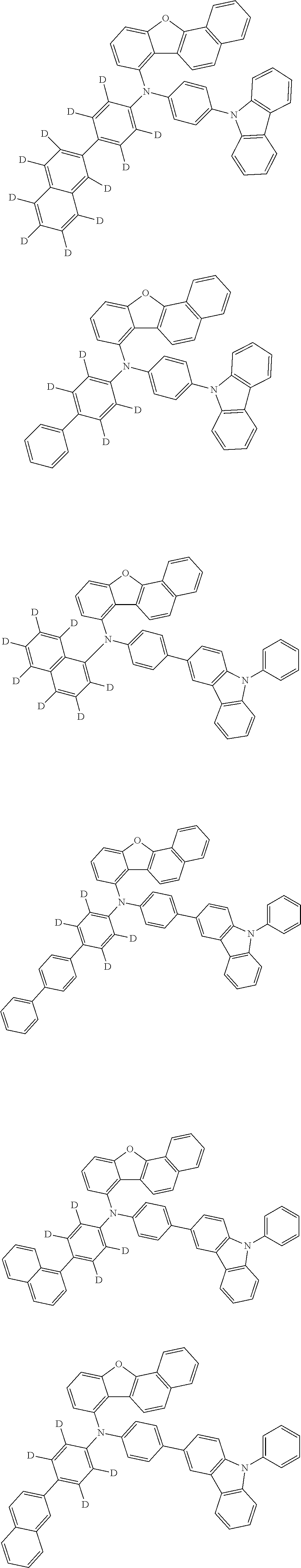

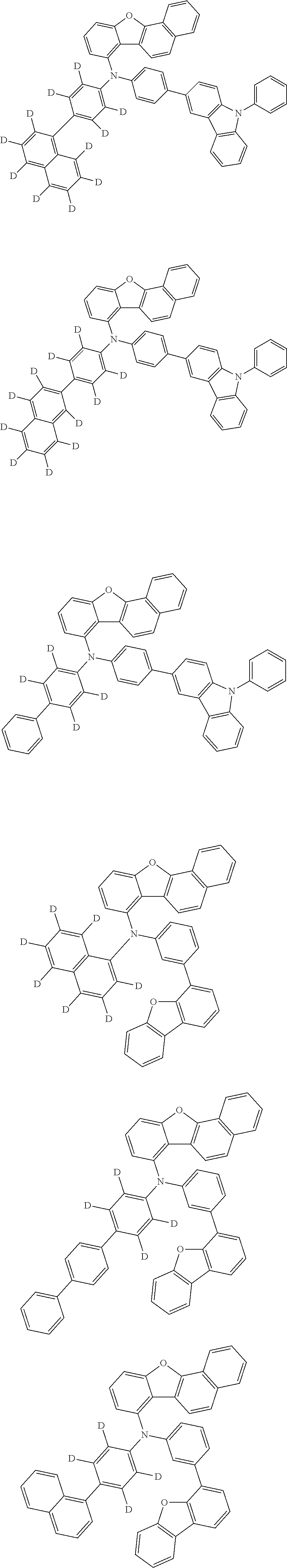

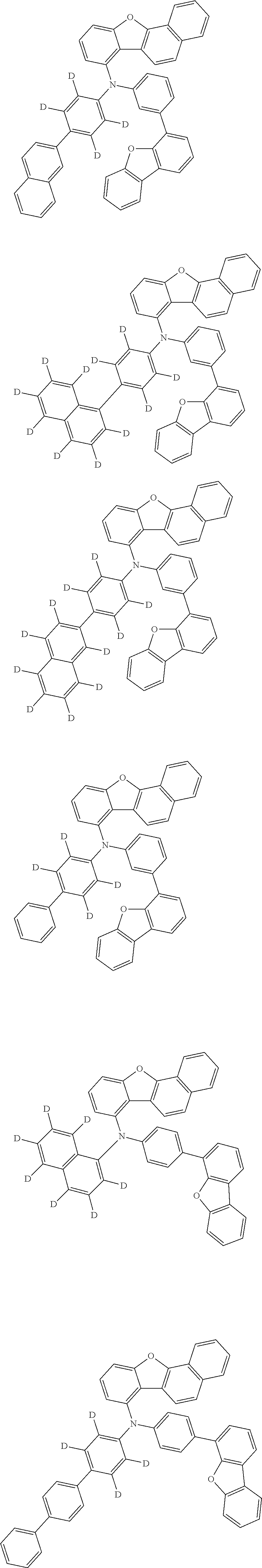

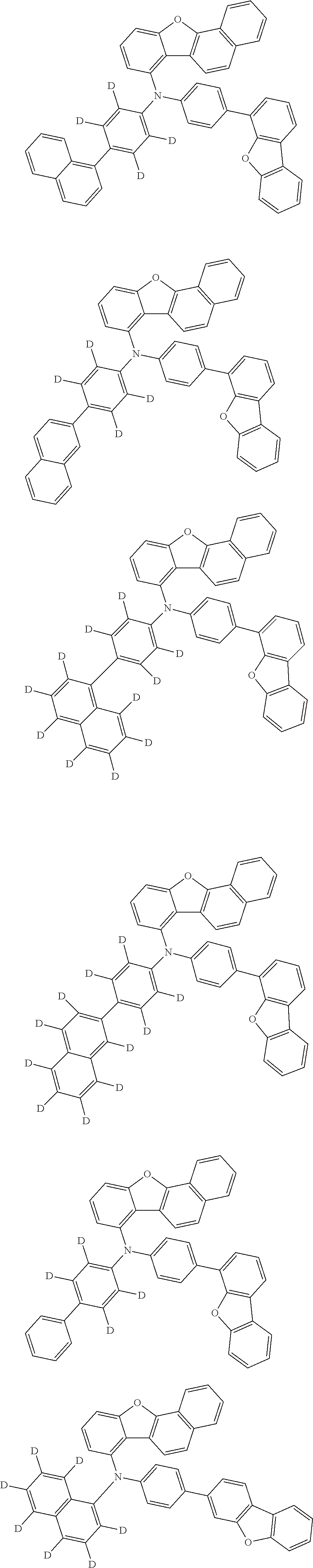

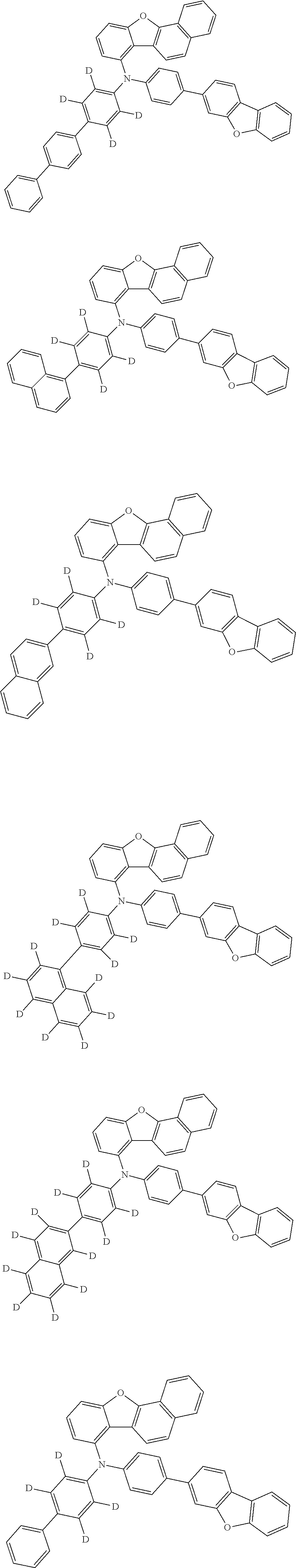

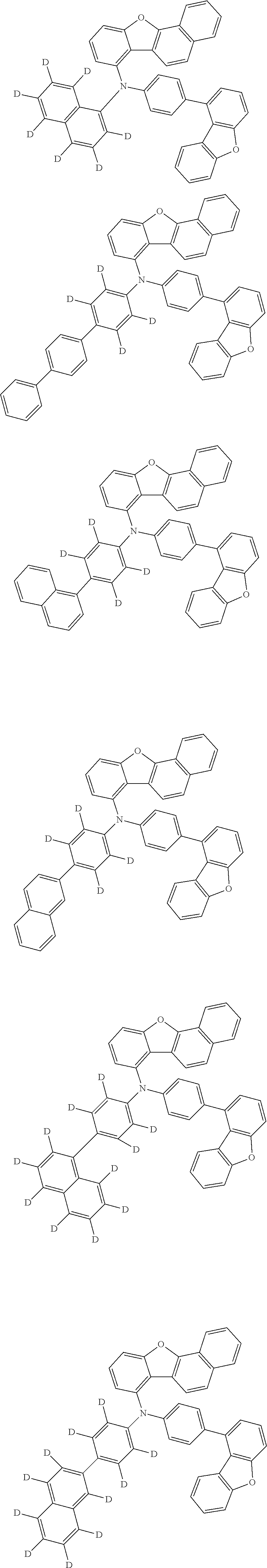

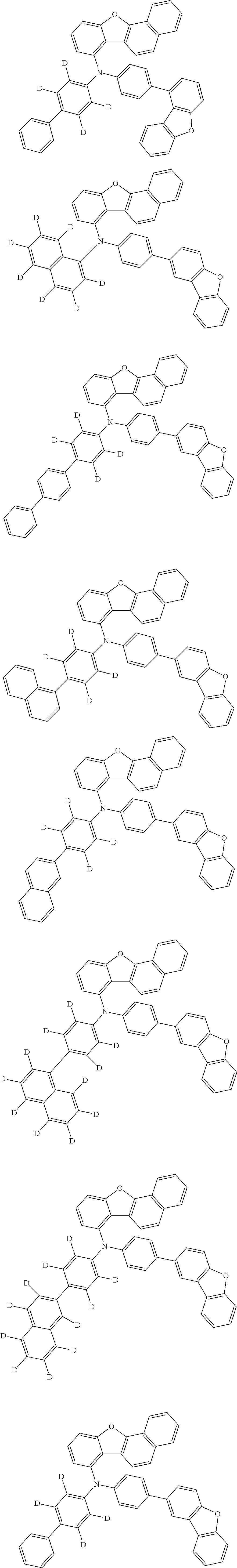

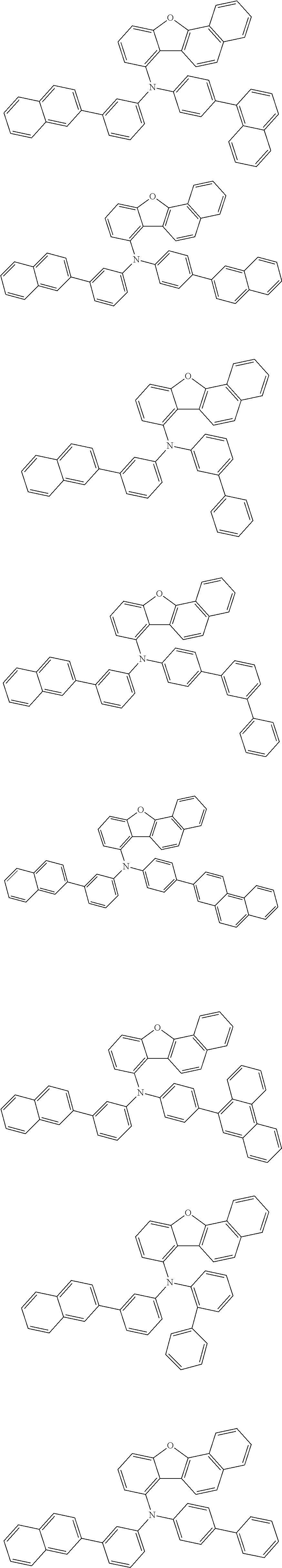

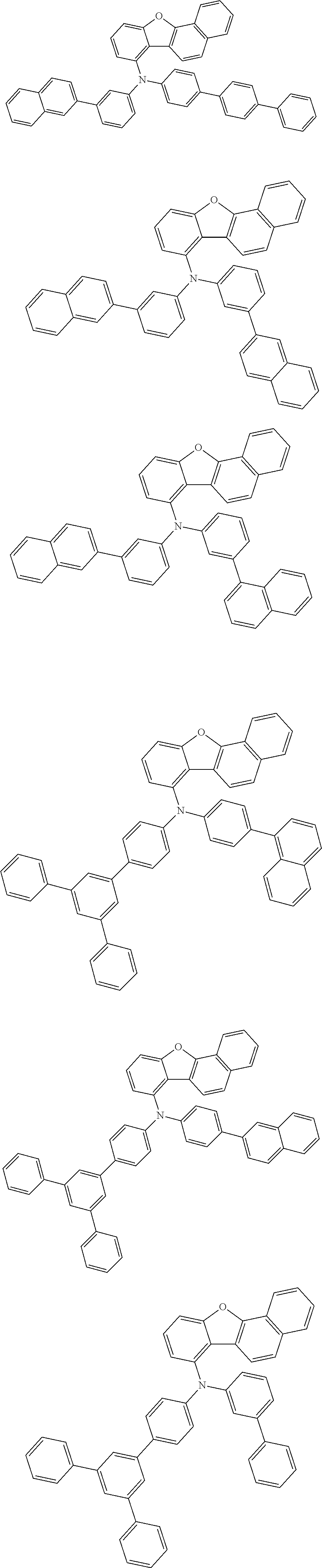

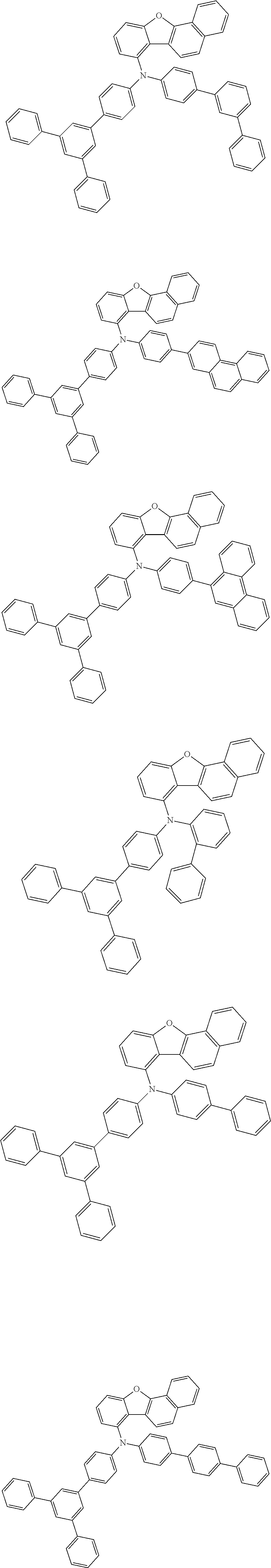

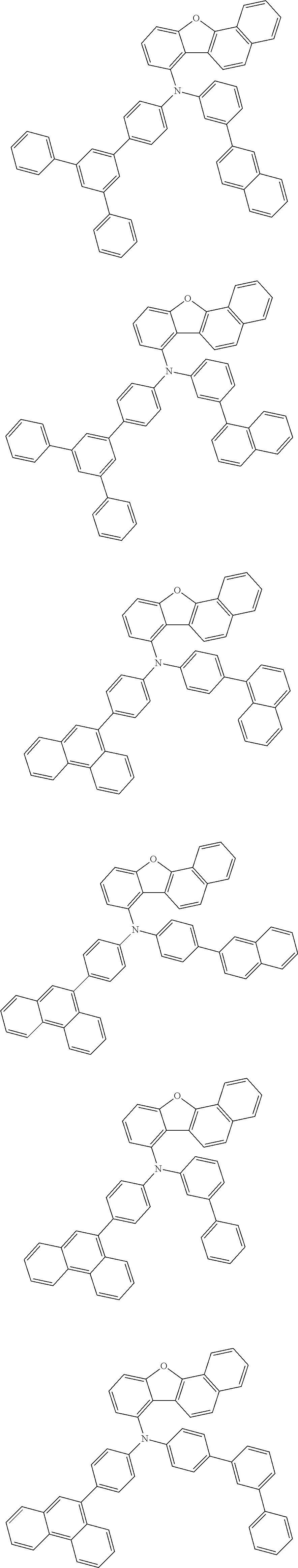

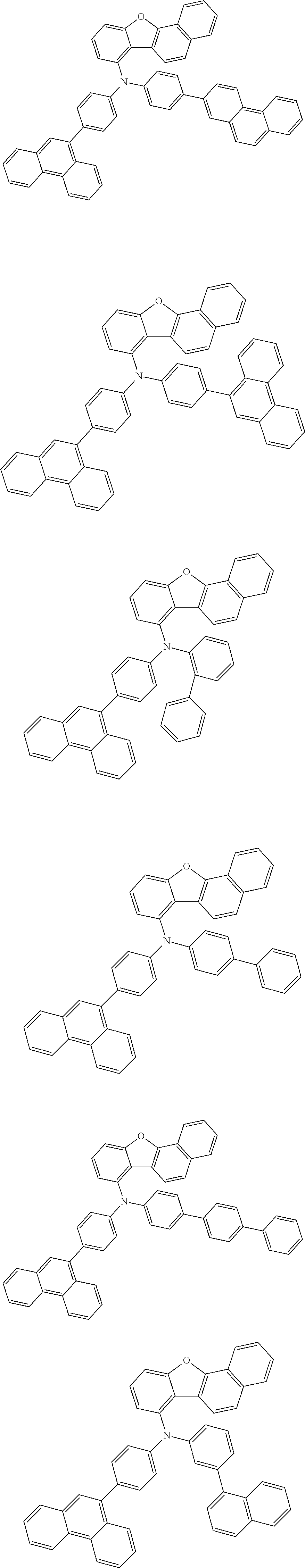

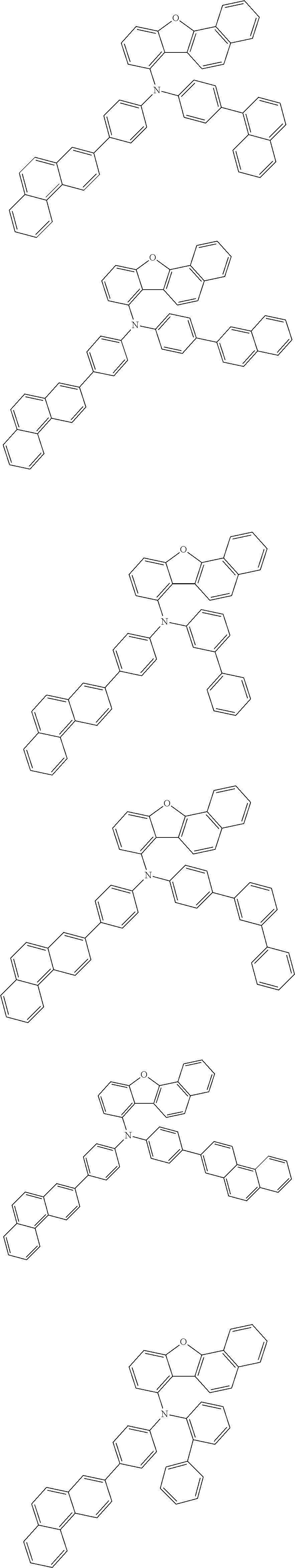

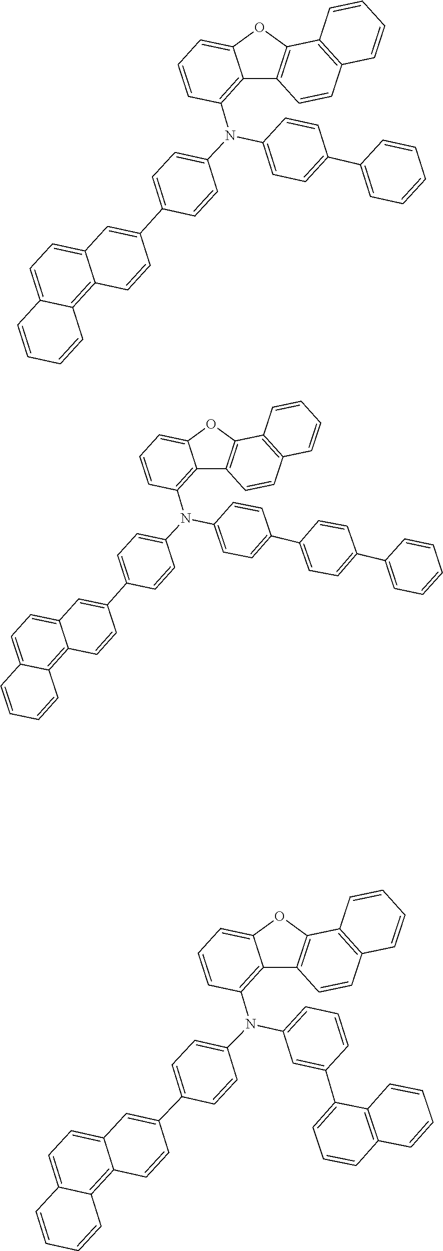 [SMILES: c1ccc(-c2ccc(N(c3ccc(-c4ccc5c(ccc6ccccc65)c4)cc3)c3cccc4oc5c6ccccc6ccc5c34)cc2)cc1 ; c1ccc(-c2ccc(-c3ccc(N(c4ccc(-c5ccc6c(ccc7ccccc76)c5)cc4)c4cccc5oc6c7ccccc7ccc6c45)cc3)cc2)cc1 ; c1cc(-c2cccc3ccccc23)cc(N(c2ccc(-c3ccc4c(ccc5ccccc54)c3)cc2)c2cccc3oc4c5ccccc5ccc4c23)c1]

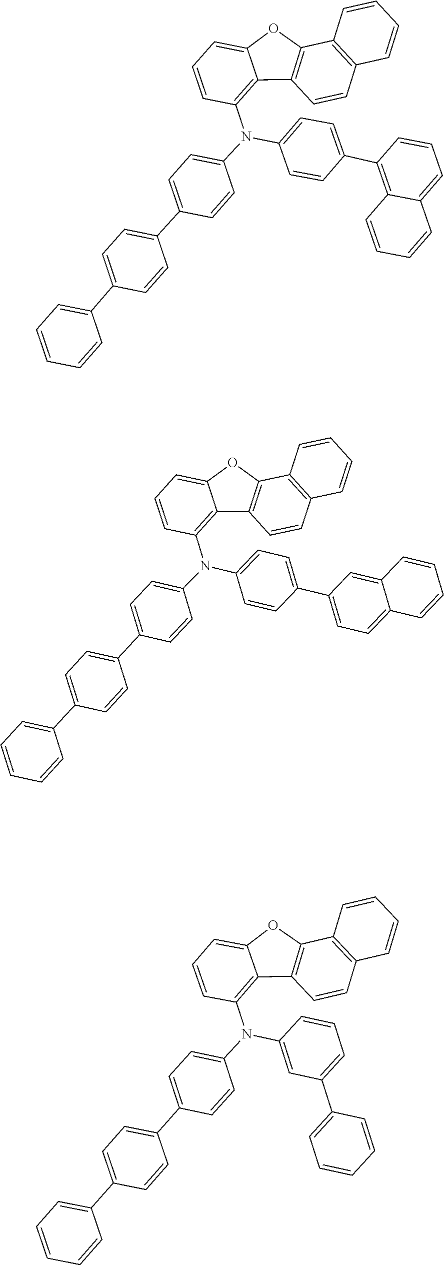 [SMILES: c1ccc(-c2ccc(-c3ccc(N(c4ccc(-c5cccc6ccccc56)cc4)c4cccc5oc6c7ccccc7ccc6c45)cc3)cc2)cc1 ; c1ccc(-c2ccc(-c3ccc(N(c4ccc(-c5ccc6ccccc6c5)cc4)c4cccc5oc6c7ccccc7ccc6c45)cc3)cc2)cc1 ; c1ccc(-c2ccc(-c3ccc(N(c4cccc(-c5ccccc5)c4)c4cccc5oc6c7ccccc7ccc6c45)cc3)cc2)cc1]

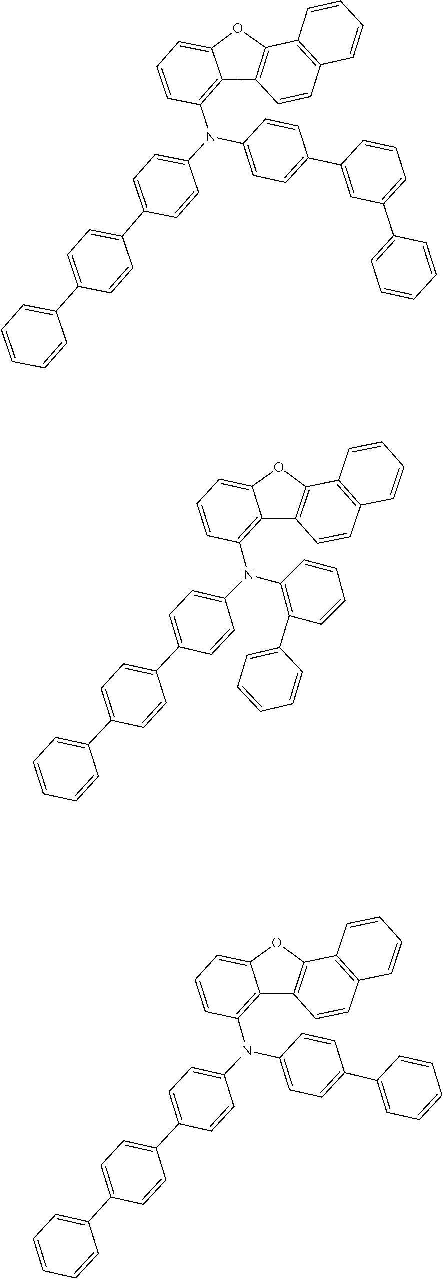 [SMILES: c1ccc(-c2ccc(-c3ccc(N(c4ccc(-c5cccc(-c6ccccc6)c5)cc4)c4cccc5oc6c7ccccc7ccc6c45)cc3)cc2)cc1 ; c1ccc(-c2ccc(-c3ccc(N(c4ccccc4-c4ccccc4)c4cccc5oc6c7ccccc7ccc6c45)cc3)cc2)cc1 ; c1ccc(-c2ccc(-c3ccc(N(c4ccc(-c5ccccc5)cc4)c4cccc5oc6c7ccccc7ccc6c45)cc3)cc2)cc1]

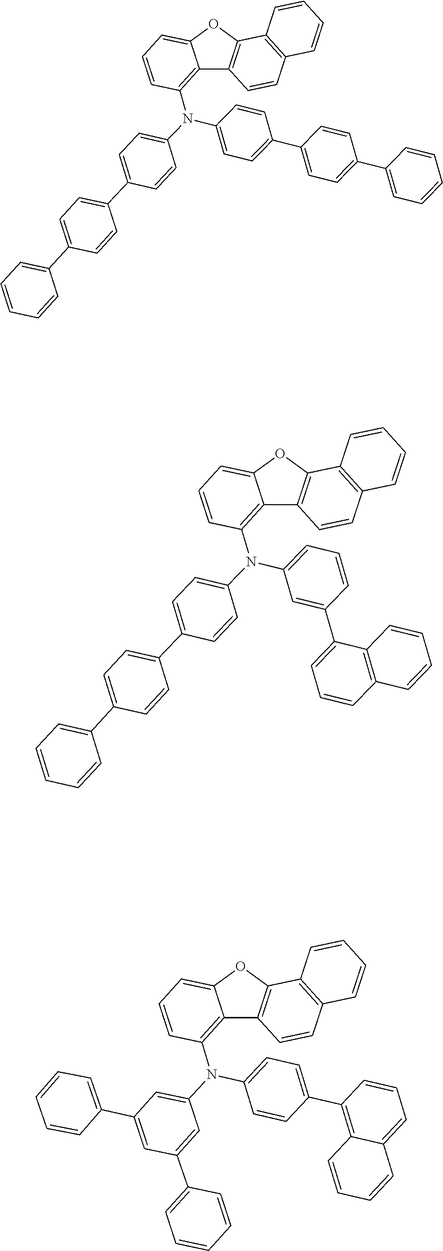 [SMILES: c1ccc(-c2ccc(-c3ccc(N(c4ccc(-c5ccc(-c6ccccc6)cc5)cc4)c4cccc5oc6c7ccccc7ccc6c45)cc3)cc2)cc1 ; c1ccc(-c2ccc(-c3ccc(N(c4cccc(-c5cccc6ccccc56)c4)c4cccc5oc6c7ccccc7ccc6c45)cc3)cc2)cc1 ; c1ccc(-c2cc(-c3ccccc3)cc(N(c3ccc(-c4cccc5ccccc45)cc3)c3cccc4oc5c6ccccc6ccc5c34)c2)cc1]

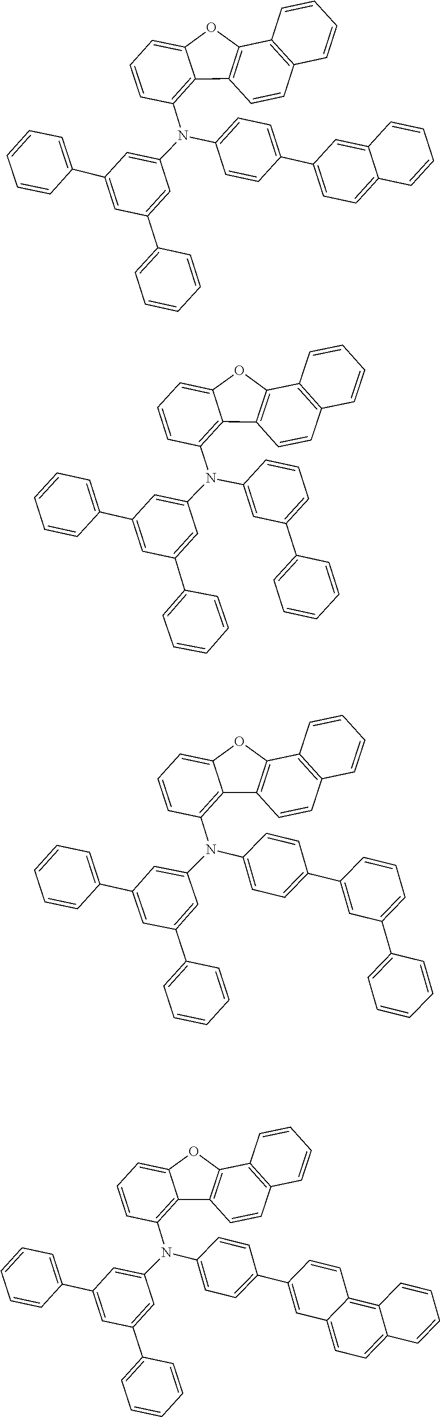 [SMILES: c1ccc(-c2cc(-c3ccccc3)cc(N(c3ccc(-c4ccc5ccccc5c4)cc3)c3cccc4oc5c6ccccc6ccc5c34)c2)cc1 ; c1ccc(-c2cccc(N(c3cc(-c4ccccc4)cc(-c4ccccc4)c3)c3cccc4oc5c6ccccc6ccc5c34)c2)cc1 ; c1ccc(-c2cccc(-c3ccc(N(c4cc(-c5ccccc5)cc(-c5ccccc5)c4)c4cccc5oc6c7ccccc7ccc6c45)cc3)c2)cc1 ; c1ccc(-c2cc(-c3ccccc3)cc(N(c3ccc(-c4ccc5c(ccc6ccccc65)c4)cc3)c3cccc4oc5c6ccccc6ccc5c34)c2)cc1]

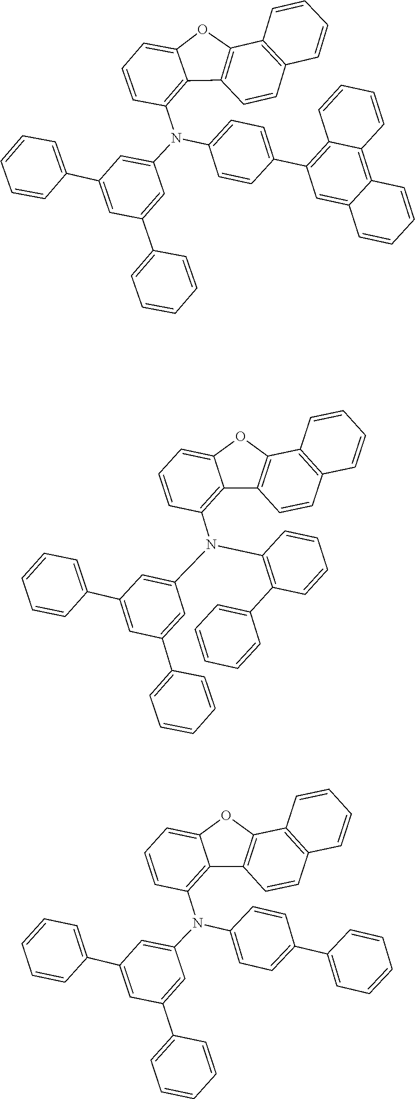 [SMILES: c1ccc(-c2cc(-c3ccccc3)cc(N(c3ccc(-c4cc5ccccc5c5ccccc45)cc3)c3cccc4oc5c6ccccc6ccc5c34)c2)cc1 ; c1ccc(-c2cc(-c3ccccc3)cc(N(c3ccccc3-c3ccccc3)c3cccc4oc5c6ccccc6ccc5c34)c2)cc1 ; c1ccc(-c2ccc(N(c3cc(-c4ccccc4)cc(-c4ccccc4)c3)c3cccc4oc5c6ccccc6ccc5c34)cc2)cc1]

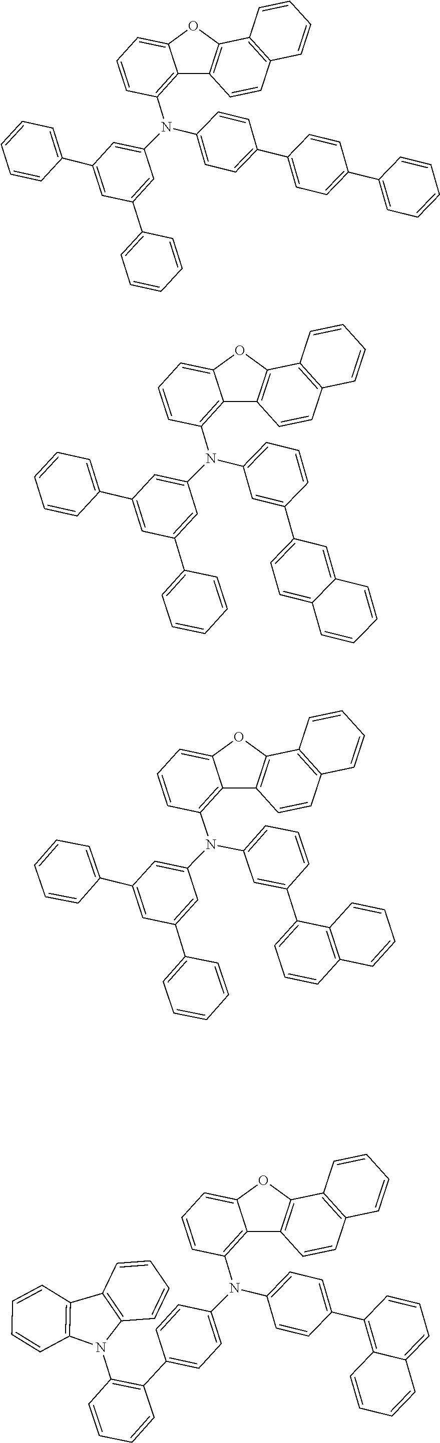 [SMILES: c1ccc(-c2ccc(-c3ccc(N(c4cc(-c5ccccc5)cc(-c5ccccc5)c4)c4cccc5oc6c7ccccc7ccc6c45)cc3)cc2)cc1 ; c1ccc(-c2cc(-c3ccccc3)cc(N(c3cccc(-c4ccc5ccccc5c4)c3)c3cccc4oc5c6ccccc6ccc5c34)c2)cc1 ; c1ccc(-c2cc(-c3ccccc3)cc(N(c3cccc(-c4cccc5ccccc45)c3)c3cccc4oc5c6ccccc6ccc5c34)c2)cc1 ; c1ccc(-n2c3ccccc3c3ccccc32)c(-c2ccc(N(c3ccc(-c4cccc5ccccc45)cc3)c3cccc4oc5c6ccccc6ccc5c34)cc2)c1]

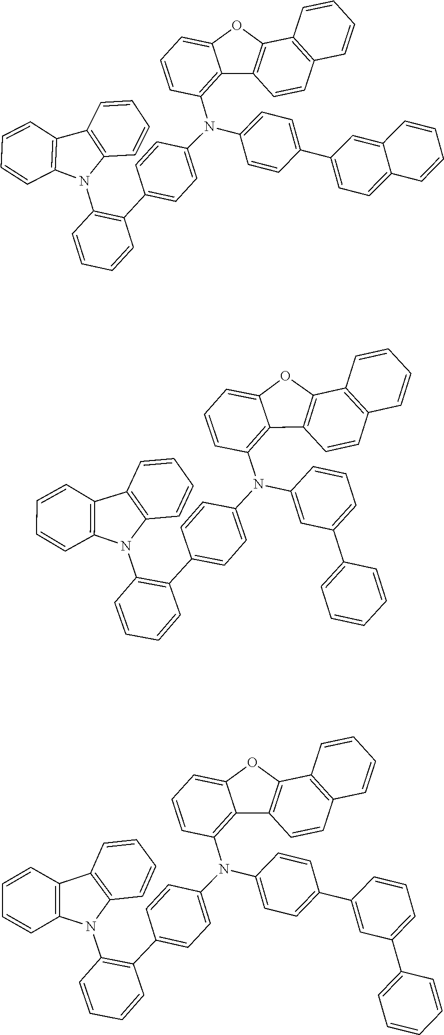 [SMILES: c1ccc(-n2c3ccccc3c3ccccc32)c(-c2ccc(N(c3ccc(-c4ccc5ccccc5c4)cc3)c3cccc4oc5c6ccccc6ccc5c34)cc2)c1 ; c1ccc(-c2cccc(N(c3ccc(-c4ccccc4-n4c5ccccc5c5ccccc54)cc3)c3cccc4oc5c6ccccc6ccc5c34)c2)cc1 ; c1ccc(-c2cccc(-c3ccc(N(c4ccc(-c5ccccc5-n5c6ccccc6c6ccccc65)cc4)c4cccc5oc6c7ccccc7ccc6c45)cc3)c2)cc1]

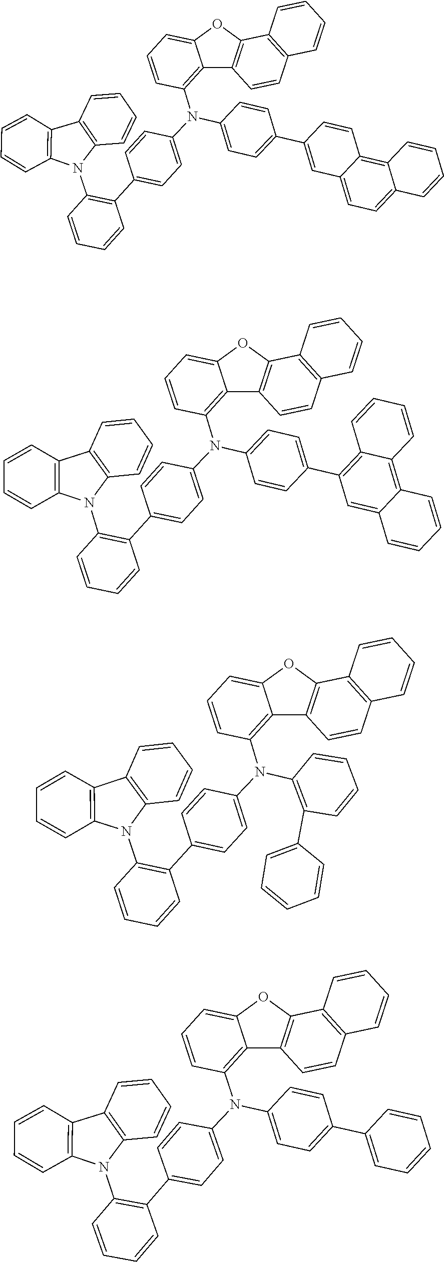 [SMILES: c1ccc(-n2c3ccccc3c3ccccc32)c(-c2ccc(N(c3ccc(-c4ccc5c(ccc6ccccc65)c4)cc3)c3cccc4oc5c6ccccc6ccc5c34)cc2)c1 ; c1ccc(-n2c3ccccc3c3ccccc32)c(-c2ccc(N(c3ccc(-c4cc5ccccc5c5ccccc45)cc3)c3cccc4oc5c6ccccc6ccc5c34)cc2)c1 ; c1ccc(-c2ccccc2N(c2ccc(-c3ccccc3-n3c4ccccc4c4ccccc43)cc2)c2cccc3oc4c5ccccc5ccc4c23)cc1 ; c1ccc(-c2ccc(N(c3ccc(-c4ccccc4-n4c5ccccc5c5ccccc54)cc3)c3cccc4oc5c6ccccc6ccc5c34)cc2)cc1]

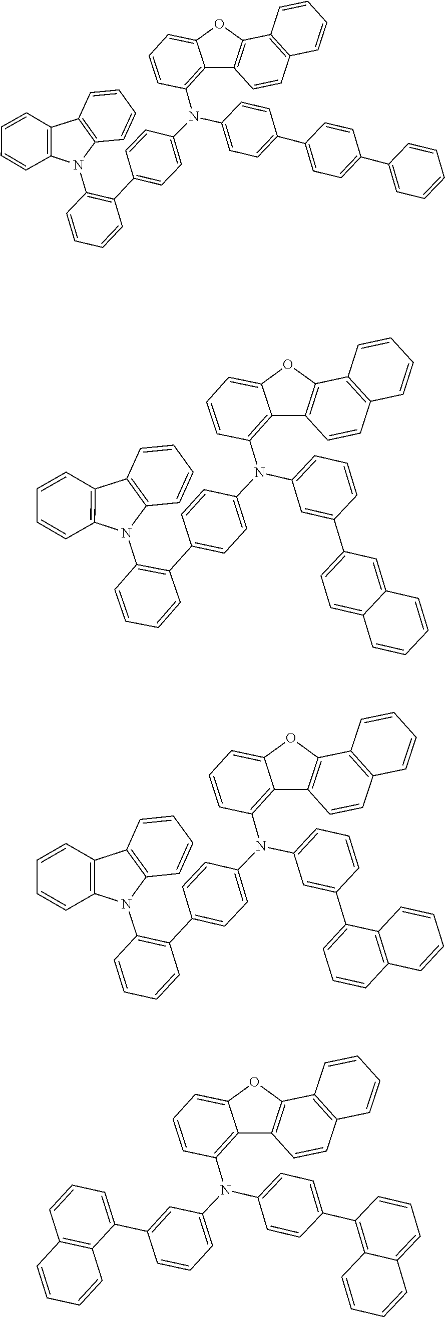 [SMILES: c1ccc(-c2ccc(-c3ccc(N(c4ccc(-c5ccccc5-n5c6ccccc6c6ccccc65)cc4)c4cccc5oc6c7ccccc7ccc6c45)cc3)cc2)cc1 ; c1cc(-c2ccc3ccccc3c2)cc(N(c2ccc(-c3ccccc3-n3c4ccccc4c4ccccc43)cc2)c2cccc3oc4c5ccccc5ccc4c23)c1 ; c1cc(-c2cccc3ccccc23)cc(N(c2ccc(-c3ccccc3-n3c4ccccc4c4ccccc43)cc2)c2cccc3oc4c5ccccc5ccc4c23)c1 ; c1cc(-c2cccc3ccccc23)cc(N(c2ccc(-c3cccc4ccccc34)cc2)c2cccc3oc4c5ccccc5ccc4c23)c1]

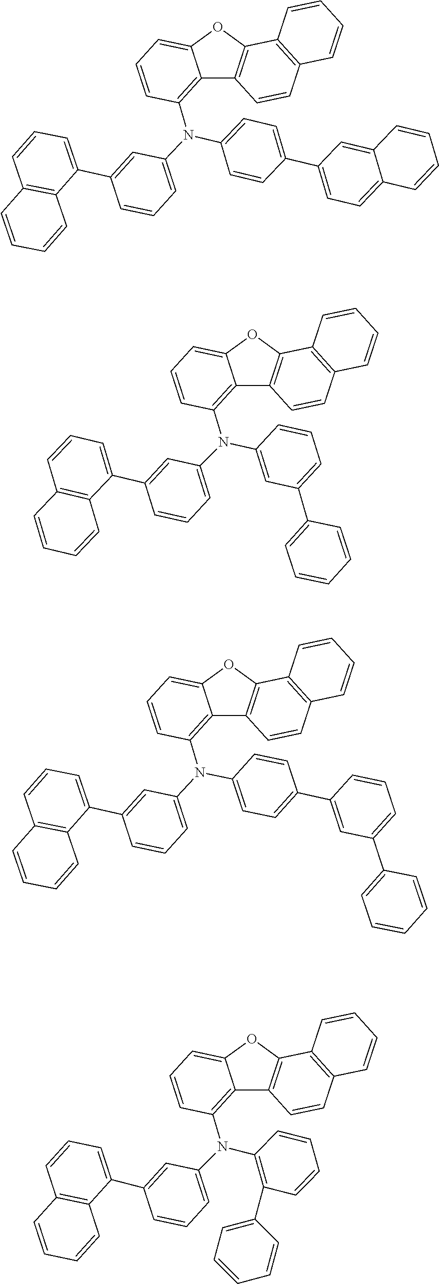 [SMILES: c1cc(-c2cccc3ccccc23)cc(N(c2ccc(-c3ccc4ccccc4c3)cc2)c2cccc3oc4c5ccccc5ccc4c23)c1 ; c1ccc(-c2cccc(N(c3cccc(-c4cccc5ccccc45)c3)c3cccc4oc5c6ccccc6ccc5c34)c2)cc1 ; c1ccc(-c2cccc(-c3ccc(N(c4cccc(-c5cccc6ccccc56)c4)c4cccc5oc6c7ccccc7ccc6c45)cc3)c2)cc1 ; c1ccc(-c2ccccc2N(c2cccc(-c3cccc4ccccc34)c2)c2cccc3oc4c5ccccc5ccc4c23)cc1]

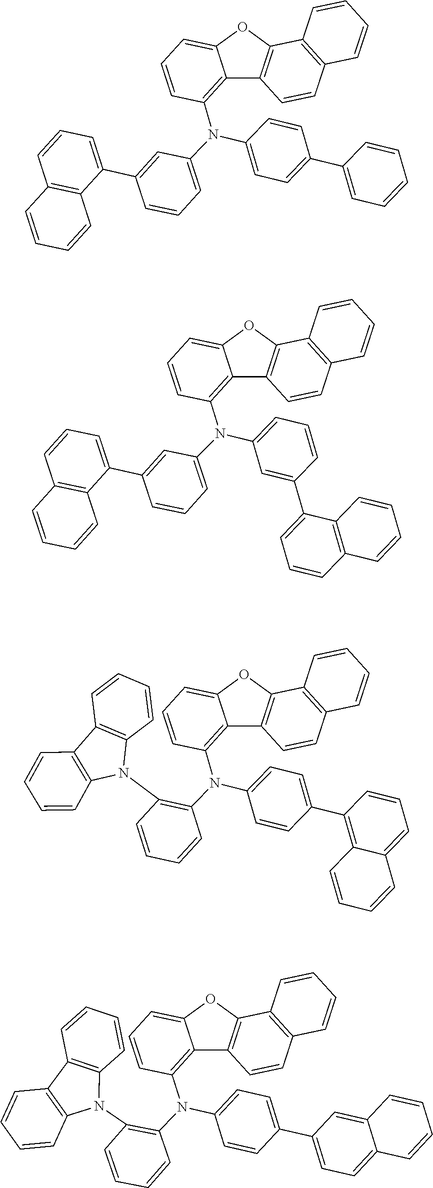 [SMILES: c1ccc(-c2ccc(N(c3cccc(-c4cccc5ccccc45)c3)c3cccc4oc5c6ccccc6ccc5c34)cc2)cc1 ; c1cc(-c2cccc3ccccc23)cc(N(c2cccc(-c3cccc4ccccc34)c2)c2cccc3oc4c5ccccc5ccc4c23)c1 ; c1ccc(-n2c3ccccc3c3ccccc32)c(N(c2ccc(-c3cccc4ccccc34)cc2)c2cccc3oc4c5ccccc5ccc4c23)c1 ; c1ccc(-n2c3ccccc3c3ccccc32)c(N(c2ccc(-c3ccc4ccccc4c3)cc2)c2cccc3oc4c5ccccc5ccc4c23)c1]

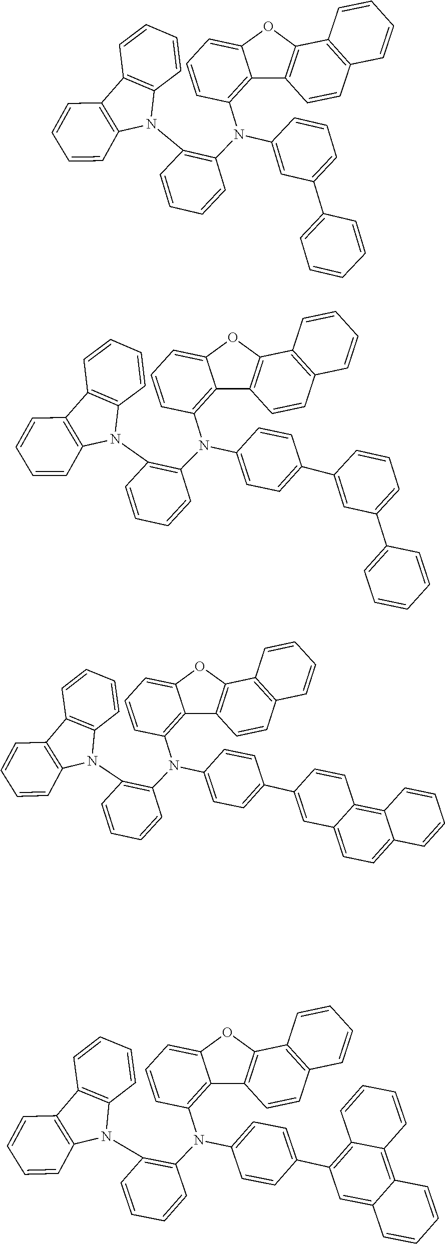

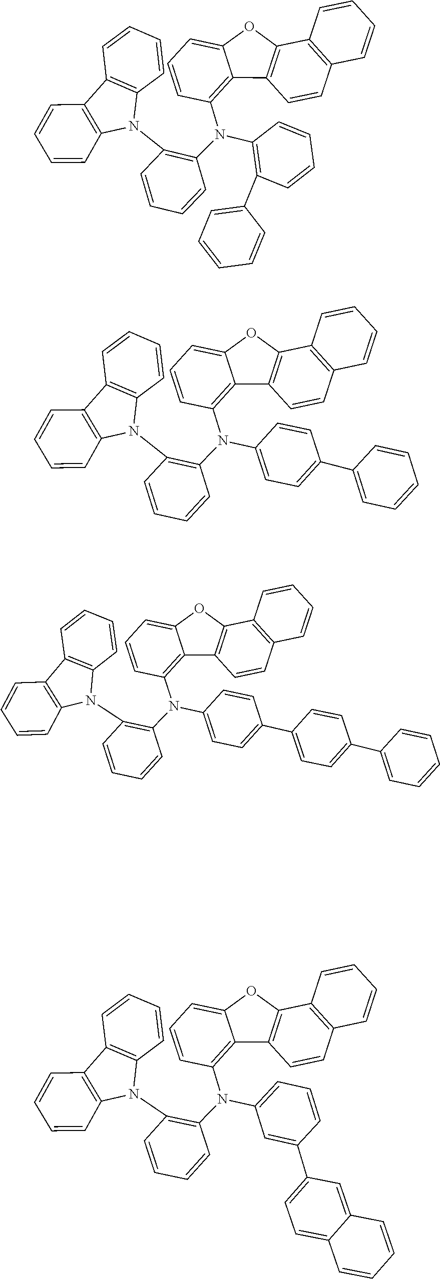 [SMILES: c1ccc(-c2ccccc2N(c2ccccc2-n2c3ccccc3c3ccccc32)c2cccc3oc4c5ccccc5ccc4c23)cc1 ; c1ccc(-c2ccc(N(c3ccccc3-n3c4ccccc4c4ccccc43)c3cccc4oc5c6ccccc6ccc5c34)cc2)cc1 ; c1ccc(-c2ccc(-c3ccc(N(c4ccccc4-n4c5ccccc5c5ccccc54)c4cccc5oc6c7ccccc7ccc6c45)cc3)cc2)cc1 ; c1cc(-c2ccc3ccccc3c2)cc(N(c2ccccc2-n2c3ccccc3c3ccccc32)c2cccc3oc4c5ccccc5ccc4c23)c1]

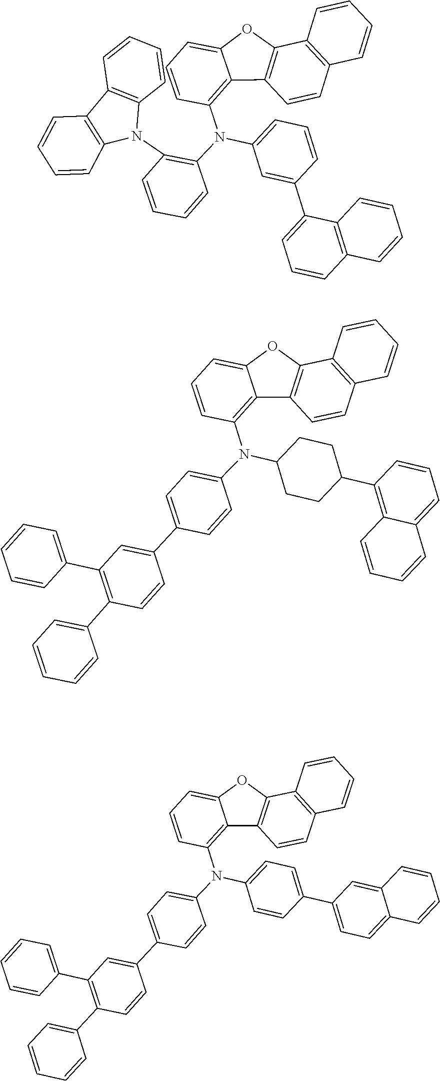 [SMILES: c1cc(-c2cccc3ccccc23)cc(N(c2ccccc2-n2c3ccccc3c3ccccc32)c2cccc3oc4c5ccccc5ccc4c23)c1 ; c1ccc(-c2ccc(-c3ccc(N(c4cccc5oc6c7ccccc7ccc6c45)C4CCC(c5cccc6ccccc56)CC4)cc3)cc2-c2ccccc2)cc1 ; c1ccc(-c2ccc(-c3ccc(N(c4ccc(-c5ccc6ccccc6c5)cc4)c4cccc5oc6c7ccccc7ccc6c45)cc3)cc2-c2ccccc2)cc1]

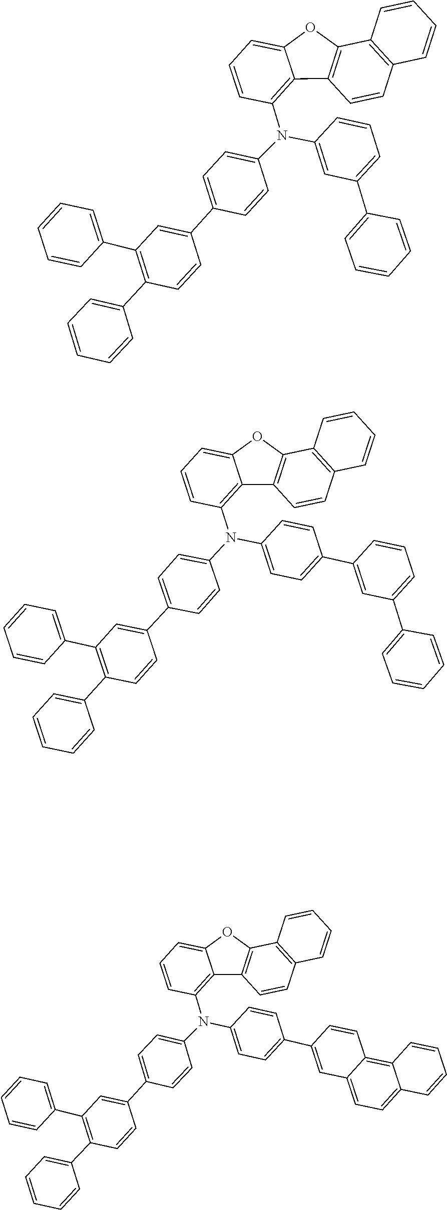 [SMILES: c1ccc(-c2cccc(N(c3ccc(-c4ccc(-c5ccccc5)c(-c5ccccc5)c4)cc3)c3cccc4oc5c6ccccc6ccc5c34)c2)cc1 ; c1ccc(-c2cccc(-c3ccc(N(c4ccc(-c5ccc(-c6ccccc6)c(-c6ccccc6)c5)cc4)c4cccc5oc6c7ccccc7ccc6c45)cc3)c2)cc1 ; c1ccc(-c2ccc(-c3ccc(N(c4ccc(-c5ccc6c(ccc7ccccc76)c5)cc4)c4cccc5oc6c7ccccc7ccc6c45)cc3)cc2-c2ccccc2)cc1]

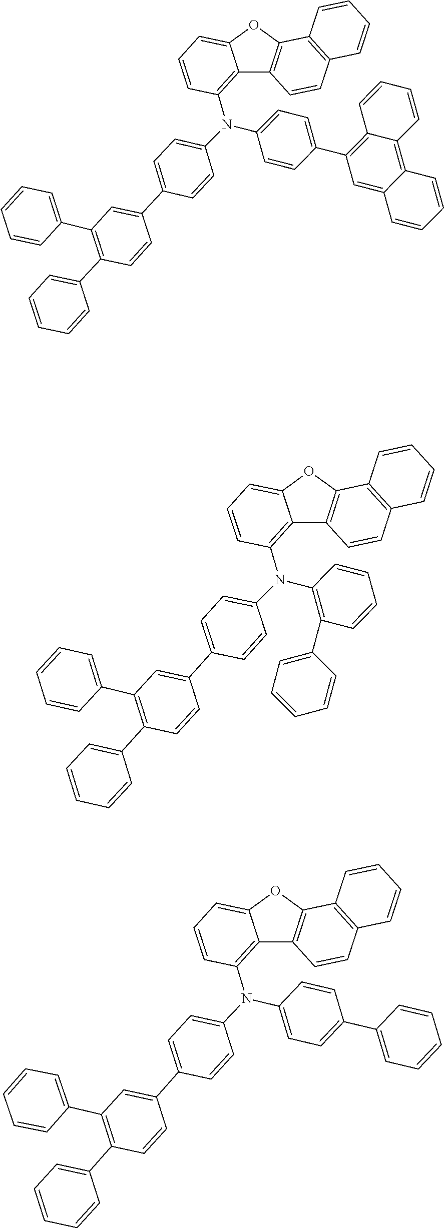 [SMILES: c1ccc(-c2ccc(-c3ccc(N(c4ccc(-c5cc6ccccc6c6ccccc56)cc4)c4cccc5oc6c7ccccc7ccc6c45)cc3)cc2-c2ccccc2)cc1 ; c1ccc(-c2ccc(-c3ccc(N(c4ccccc4-c4ccccc4)c4cccc5oc6c7ccccc7ccc6c45)cc3)cc2-c2ccccc2)cc1 ; c1ccc(-c2ccc(N(c3ccc(-c4ccc(-c5ccccc5)c(-c5ccccc5)c4)cc3)c3cccc4oc5c6ccccc6ccc5c34)cc2)cc1]

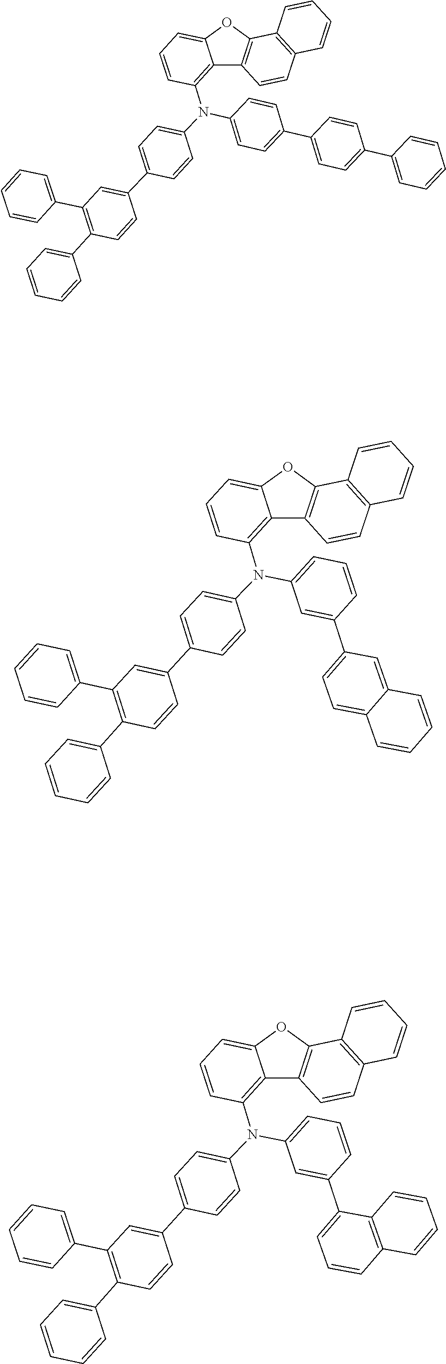 [SMILES: c1ccc(-c2ccc(-c3ccc(N(c4ccc(-c5ccc(-c6ccccc6)c(-c6ccccc6)c5)cc4)c4cccc5oc6c7ccccc7ccc6c45)cc3)cc2)cc1 ; c1ccc(-c2ccc(-c3ccc(N(c4cccc(-c5ccc6ccccc6c5)c4)c4cccc5oc6c7ccccc7ccc6c45)cc3)cc2-c2ccccc2)cc1 ; c1ccc(-c2ccc(-c3ccc(N(c4cccc(-c5cccc6ccccc56)c4)c4cccc5oc6c7ccccc7ccc6c45)cc3)cc2-c2ccccc2)cc1]

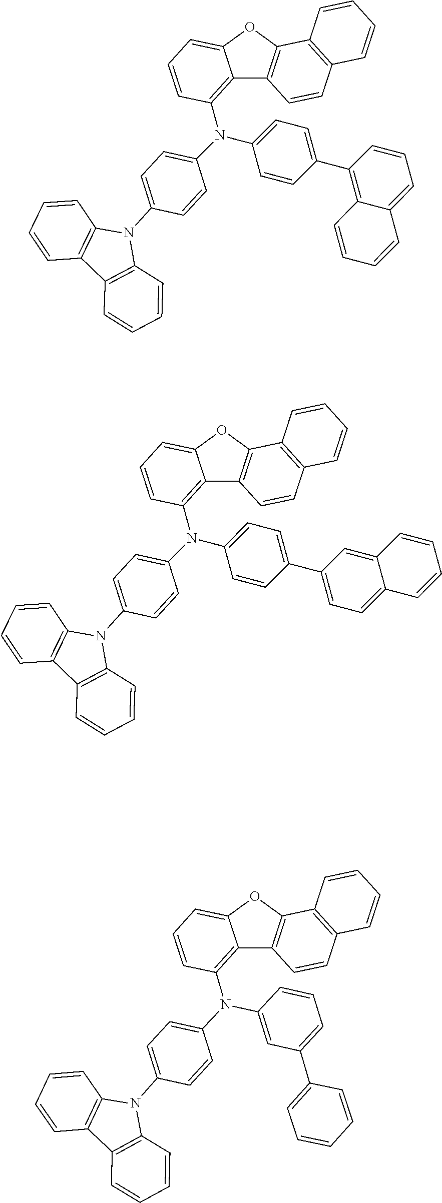 [SMILES: c1ccc2c(-c3ccc(N(c4ccc(-n5c6ccccc6c6ccccc65)cc4)c4cccc5oc6c7ccccc7ccc6c45)cc3)cccc2c1 ; c1ccc2cc(-c3ccc(N(c4ccc(-n5c6ccccc6c6ccccc65)cc4)c4cccc5oc6c7ccccc7ccc6c45)cc3)ccc2c1 ; c1ccc(-c2cccc(N(c3ccc(-n4c5ccccc5c5ccccc54)cc3)c3cccc4oc5c6ccccc6ccc5c34)c2)cc1]

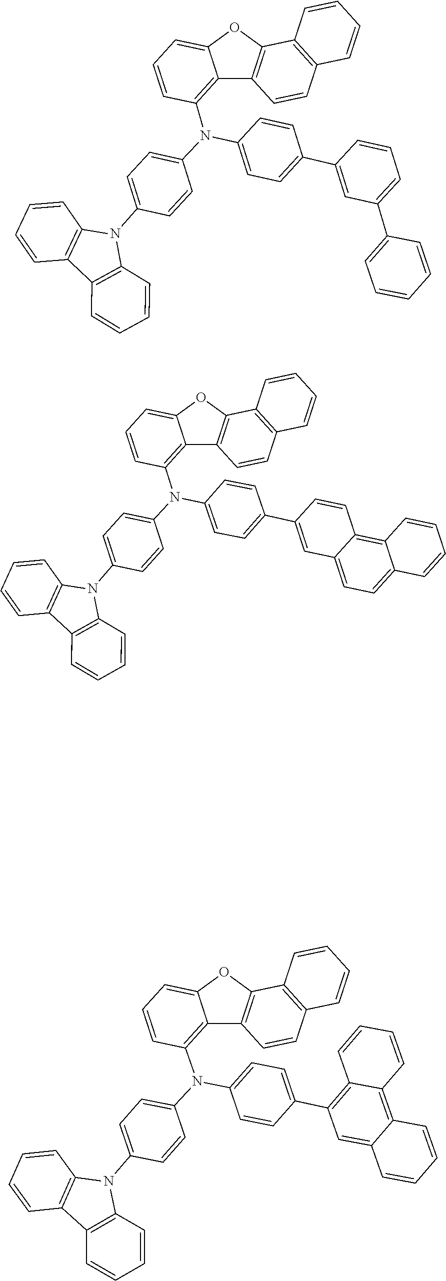 [SMILES: c1ccc(-c2cccc(-c3ccc(N(c4ccc(-n5c6ccccc6c6ccccc65)cc4)c4cccc5oc6c7ccccc7ccc6c45)cc3)c2)cc1 ; c1ccc2c(c1)ccc1cc(-c3ccc(N(c4ccc(-n5c6ccccc6c6ccccc65)cc4)c4cccc5oc6c7ccccc7ccc6c45)cc3)ccc12 ; c1ccc2c(c1)cc(-c1ccc(N(c3ccc(-n4c5ccccc5c5ccccc54)cc3)c3cccc4oc5c6ccccc6ccc5c34)cc1)c1ccccc12]

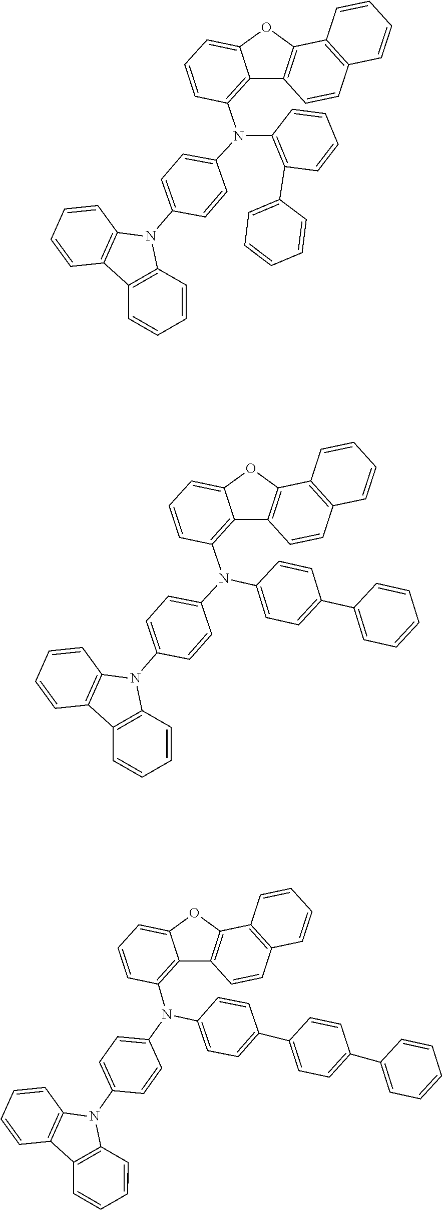 [SMILES: c1ccc(-c2ccccc2N(c2ccc(-n3c4ccccc4c4ccccc43)cc2)c2cccc3oc4c5ccccc5ccc4c23)cc1 ; c1ccc(-c2ccc(N(c3ccc(-n4c5ccccc5c5ccccc54)cc3)c3cccc4oc5c6ccccc6ccc5c34)cc2)cc1 ; c1ccc(-c2ccc(-c3ccc(N(c4ccc(-n5c6ccccc6c6ccccc65)cc4)c4cccc5oc6c7ccccc7ccc6c45)cc3)cc2)cc1]

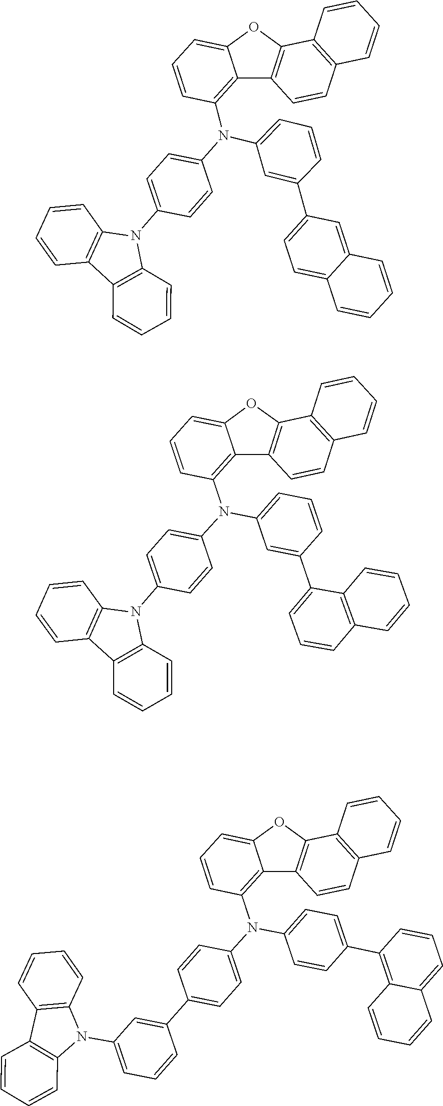 [SMILES: c1cc(-c2ccc3ccccc3c2)cc(N(c2ccc(-n3c4ccccc4c4ccccc43)cc2)c2cccc3oc4c5ccccc5ccc4c23)c1 ; c1cc(-c2cccc3ccccc23)cc(N(c2ccc(-n3c4ccccc4c4ccccc43)cc2)c2cccc3oc4c5ccccc5ccc4c23)c1 ; c1cc(-c2ccc(N(c3ccc(-c4cccc5ccccc45)cc3)c3cccc4oc5c6ccccc6ccc5c34)cc2)cc(-n2c3ccccc3c3ccccc32)c1]

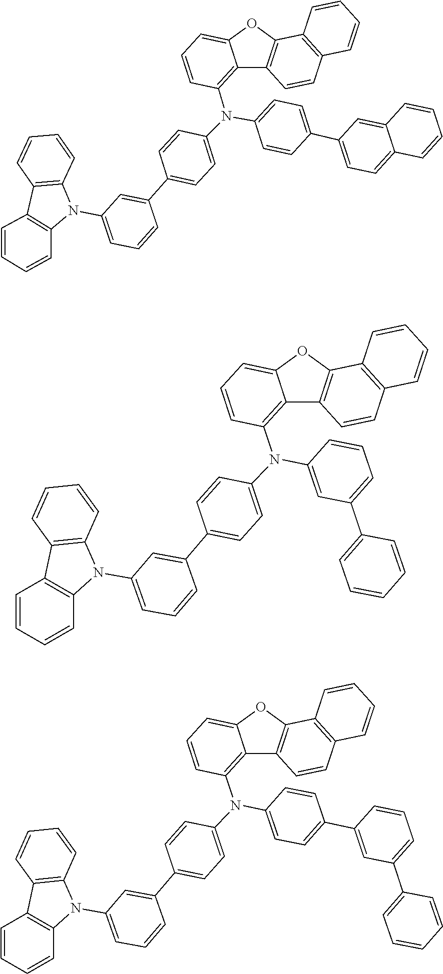 [SMILES: c1cc(-c2ccc(N(c3ccc(-c4ccc5ccccc5c4)cc3)c3cccc4oc5c6ccccc6ccc5c34)cc2)cc(-n2c3ccccc3c3ccccc32)c1 ; c1ccc(-c2cccc(N(c3ccc(-c4cccc(-n5c6ccccc6c6ccccc65)c4)cc3)c3cccc4oc5c6ccccc6ccc5c34)c2)cc1 ; c1ccc(-c2cccc(-c3ccc(N(c4ccc(-c5cccc(-n6c7ccccc7c7ccccc76)c5)cc4)c4cccc5oc6c7ccccc7ccc6c45)cc3)c2)cc1]

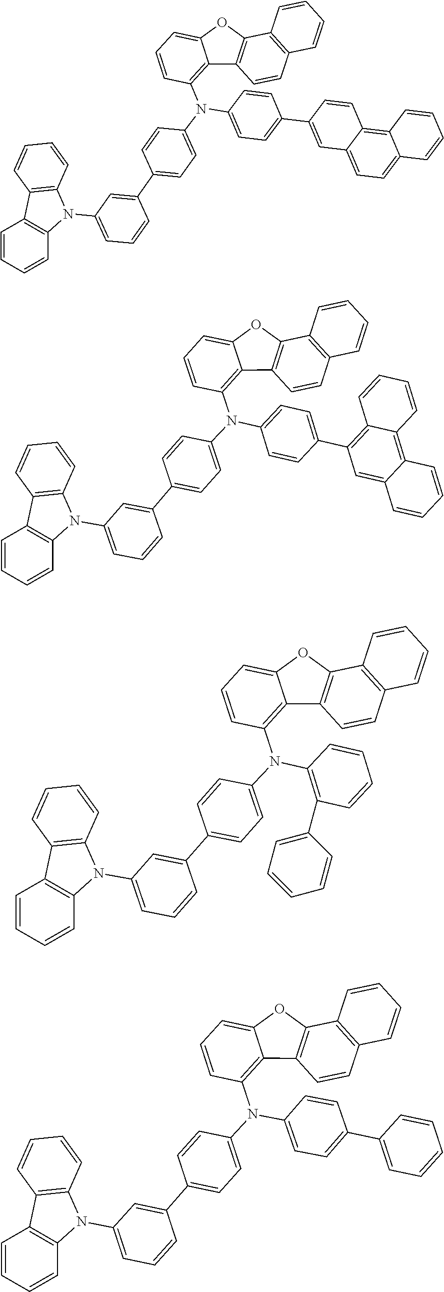 [SMILES: c1cc(-c2ccc(N(c3ccc(-c4ccc5c(ccc6ccccc65)c4)cc3)c3cccc4oc5c6ccccc6ccc5c34)cc2)cc(-n2c3ccccc3c3ccccc32)c1 ; c1cc(-c2ccc(N(c3ccc(-c4cc5ccccc5c5ccccc45)cc3)c3cccc4oc5c6ccccc6ccc5c34)cc2)cc(-n2c3ccccc3c3ccccc32)c1 ; c1ccc(-c2ccccc2N(c2ccc(-c3cccc(-n4c5ccccc5c5ccccc54)c3)cc2)c2cccc3oc4c5ccccc5ccc4c23)cc1 ; c1ccc(-c2ccc(N(c3ccc(-c4cccc(-n5c6ccccc6c6ccccc65)c4)cc3)c3cccc4oc5c6ccccc6ccc5c34)cc2)cc1]

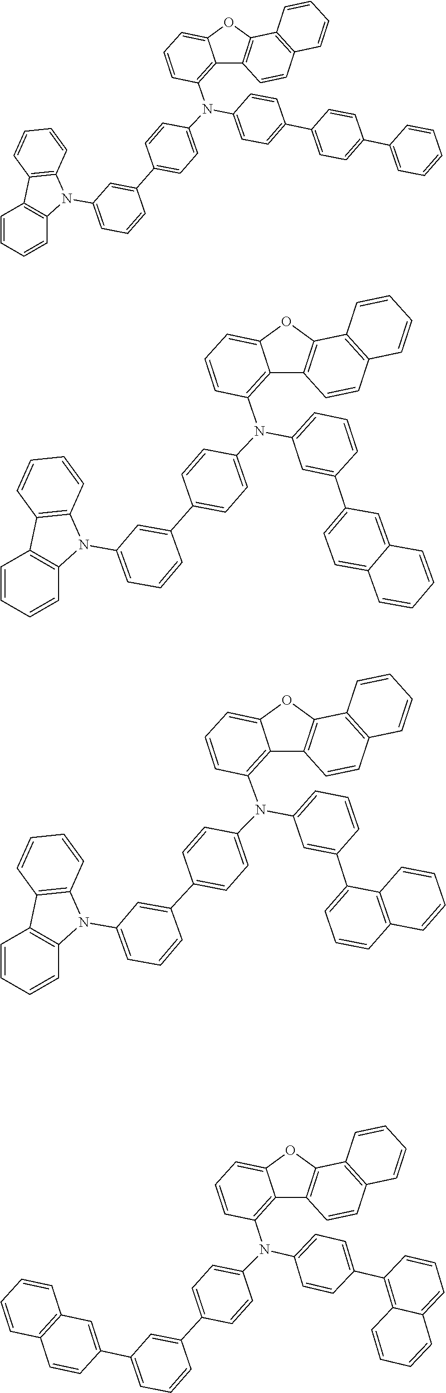 [SMILES: c1ccc(-c2ccc(-c3ccc(N(c4ccc(-c5cccc(-n6c7ccccc7c7ccccc76)c5)cc4)c4cccc5oc6c7ccccc7ccc6c45)cc3)cc2)cc1 ; c1cc(-c2ccc3ccccc3c2)cc(N(c2ccc(-c3cccc(-n4c5ccccc5c5ccccc54)c3)cc2)c2cccc3oc4c5ccccc5ccc4c23)c1 ; c1cc(-c2cccc3ccccc23)cc(N(c2ccc(-c3cccc(-n4c5ccccc5c5ccccc54)c3)cc2)c2cccc3oc4c5ccccc5ccc4c23)c1 ; c1cc(-c2ccc(N(c3ccc(-c4cccc5ccccc45)cc3)c3cccc4oc5c6ccccc6ccc5c34)cc2)cc(-c2ccc3ccccc3c2)c1]

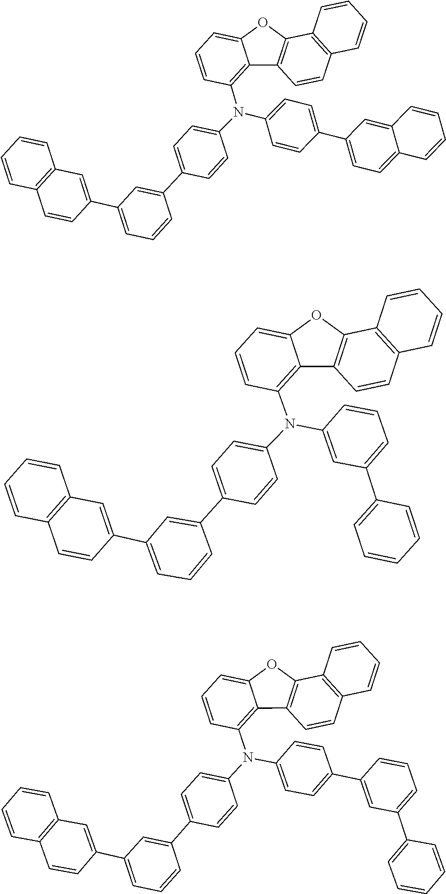 [SMILES: c1cc(-c2ccc(N(c3ccc(-c4ccc5ccccc5c4)cc3)c3cccc4oc5c6ccccc6ccc5c34)cc2)cc(-c2ccc3ccccc3c2)c1 ; c1ccc(-c2cccc(N(c3ccc(-c4cccc(-c5ccc6ccccc6c5)c4)cc3)c3cccc4oc5c6ccccc6ccc5c34)c2)cc1 ; c1ccc(-c2cccc(-c3ccc(N(c4ccc(-c5cccc(-c6ccc7ccccc7c6)c5)cc4)c4cccc5oc6c7ccccc7ccc6c45)cc3)c2)cc1]

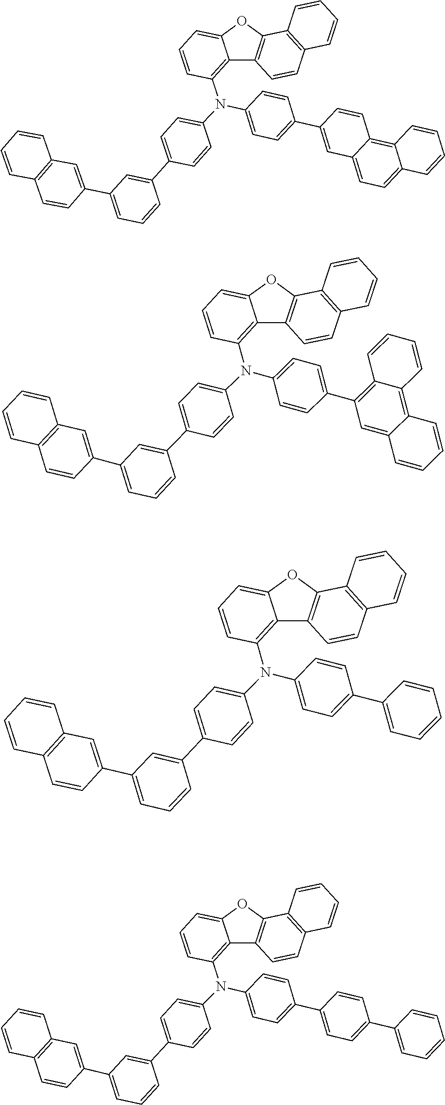 [SMILES: c1cc(-c2ccc(N(c3ccc(-c4ccc5c(ccc6ccccc65)c4)cc3)c3cccc4oc5c6ccccc6ccc5c34)cc2)cc(-c2ccc3ccccc3c2)c1 ; c1cc(-c2ccc(N(c3ccc(-c4cc5ccccc5c5ccccc45)cc3)c3cccc4oc5c6ccccc6ccc5c34)cc2)cc(-c2ccc3ccccc3c2)c1 ; c1ccc(-c2ccc(N(c3ccc(-c4cccc(-c5ccc6ccccc6c5)c4)cc3)c3cccc4oc5c6ccccc6ccc5c34)cc2)cc1 ; c1ccc(-c2ccc(-c3ccc(N(c4ccc(-c5cccc(-c6ccc7ccccc7c6)c5)cc4)c4cccc5oc6c7ccccc7ccc6c45)cc3)cc2)cc1]

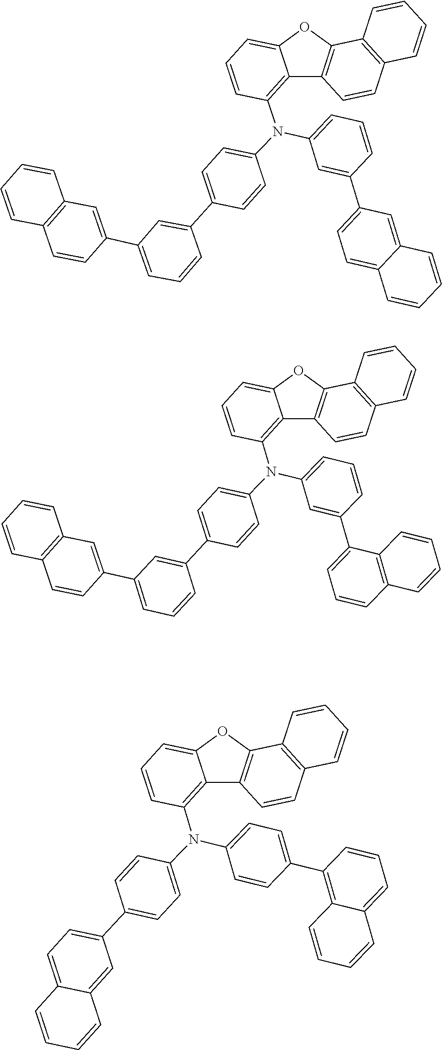 [SMILES: c1cc(-c2ccc(N(c3cccc(-c4ccc5ccccc5c4)c3)c3cccc4oc5c6ccccc6ccc5c34)cc2)cc(-c2ccc3ccccc3c2)c1 ; c1cc(-c2ccc(N(c3cccc(-c4cccc5ccccc45)c3)c3cccc4oc5c6ccccc6ccc5c34)cc2)cc(-c2ccc3ccccc3c2)c1 ; c1ccc2cc(-c3ccc(N(c4ccc(-c5cccc6ccccc56)cc4)c4cccc5oc6c7ccccc7ccc6c45)cc3)ccc2c1]

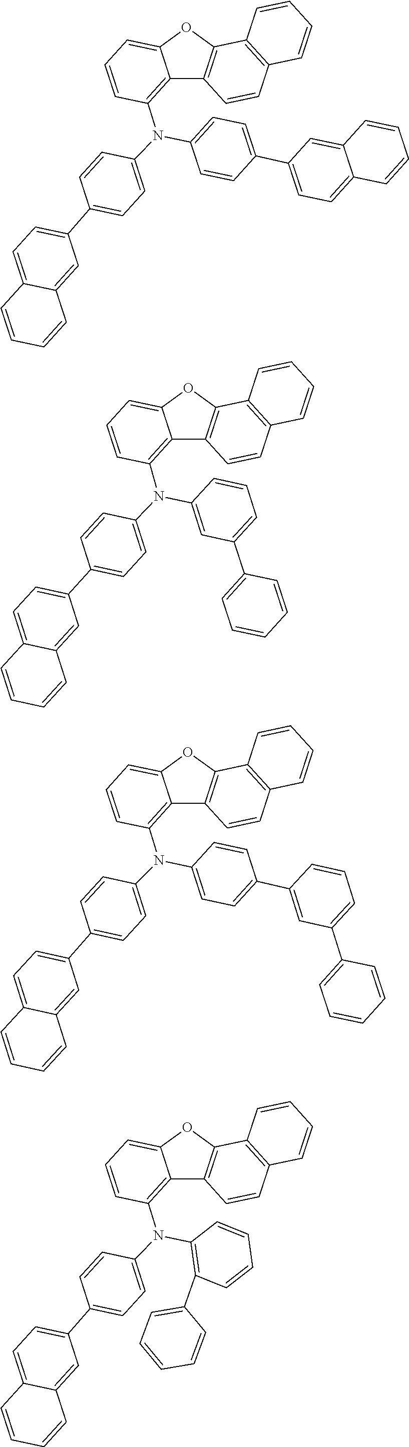 [SMILES: c1ccc2cc(-c3ccc(N(c4ccc(-c5ccc6ccccc6c5)cc4)c4cccc5oc6c7ccccc7ccc6c45)cc3)ccc2c1 ; c1ccc(-c2cccc(N(c3ccc(-c4ccc5ccccc5c4)cc3)c3cccc4oc5c6ccccc6ccc5c34)c2)cc1 ; c1ccc(-c2cccc(-c3ccc(N(c4ccc(-c5ccc6ccccc6c5)cc4)c4cccc5oc6c7ccccc7ccc6c45)cc3)c2)cc1 ; c1ccc(-c2ccccc2N(c2ccc(-c3ccc4ccccc4c3)cc2)c2cccc3oc4c5ccccc5ccc4c23)cc1]

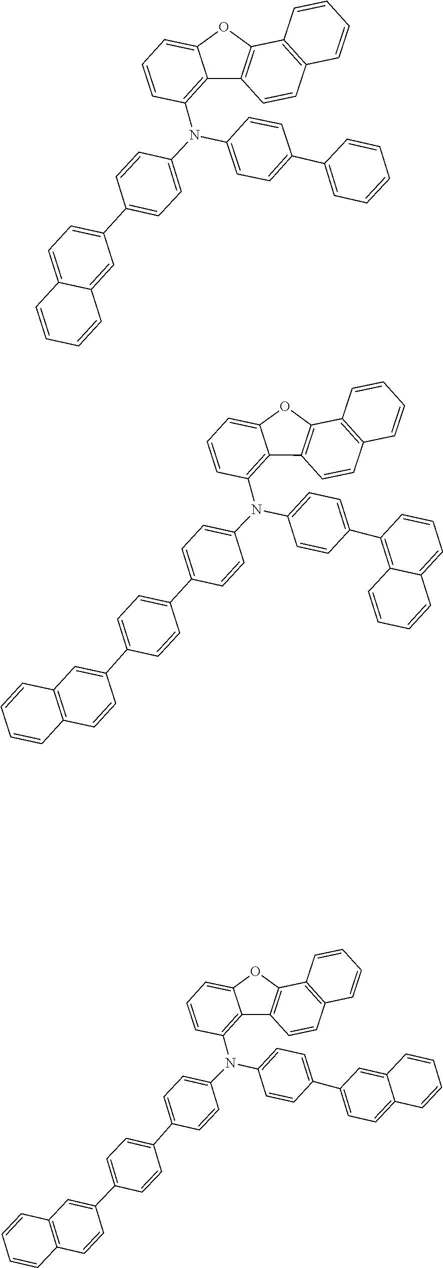 [SMILES: c1ccc(-c2ccc(N(c3ccc(-c4ccc5ccccc5c4)cc3)c3cccc4oc5c6ccccc6ccc5c34)cc2)cc1 ; c1ccc2cc(-c3ccc(-c4ccc(N(c5ccc(-c6cccc7ccccc67)cc5)c5cccc6oc7c8ccccc8ccc7c56)cc4)cc3)ccc2c1 ; c1ccc2cc(-c3ccc(-c4ccc(N(c5ccc(-c6ccc7ccccc7c6)cc5)c5cccc6oc7c8ccccc8ccc7c56)cc4)cc3)ccc2c1]

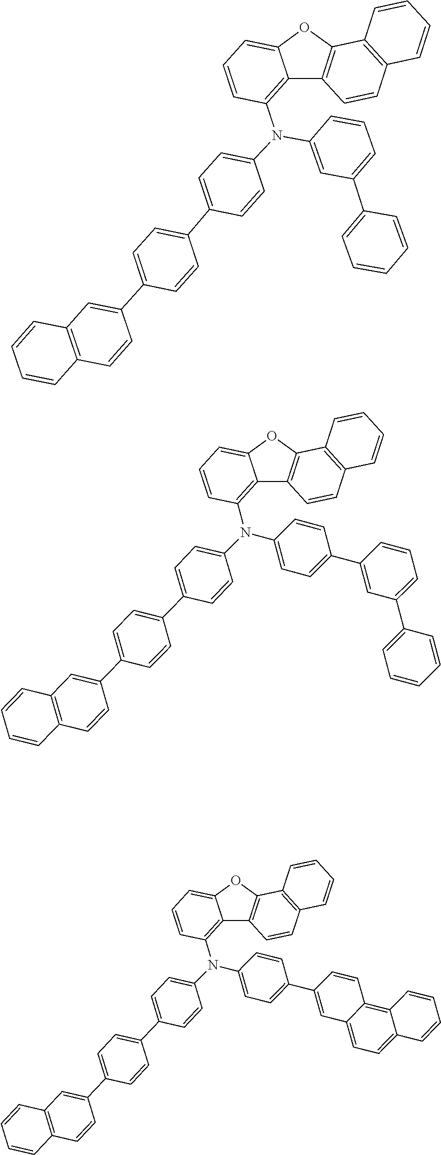 [SMILES: c1ccc(-c2cccc(N(c3ccc(-c4ccc(-c5ccc6ccccc6c5)cc4)cc3)c3cccc4oc5c6ccccc6ccc5c34)c2)cc1 ; c1ccc(-c2cccc(-c3ccc(N(c4ccc(-c5ccc(-c6ccc7ccccc7c6)cc5)cc4)c4cccc5oc6c7ccccc7ccc6c45)cc3)c2)cc1 ; c1ccc2cc(-c3ccc(-c4ccc(N(c5ccc(-c6ccc7c(ccc8ccccc87)c6)cc5)c5cccc6oc7c8ccccc8ccc7c56)cc4)cc3)ccc2c1]

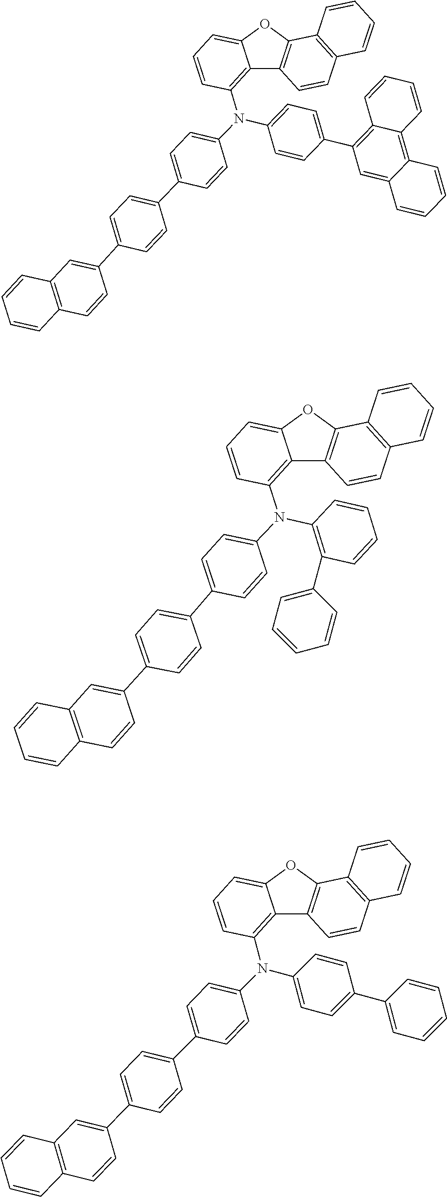 [SMILES: c1ccc2cc(-c3ccc(-c4ccc(N(c5ccc(-c6cc7ccccc7c7ccccc67)cc5)c5cccc6oc7c8ccccc8ccc7c56)cc4)cc3)ccc2c1 ; c1ccc(-c2ccccc2N(c2ccc(-c3ccc(-c4ccc5ccccc5c4)cc3)cc2)c2cccc3oc4c5ccccc5ccc4c23)cc1 ; c1ccc(-c2ccc(N(c3ccc(-c4ccc(-c5ccc6ccccc6c5)cc4)cc3)c3cccc4oc5c6ccccc6ccc5c34)cc2)cc1]

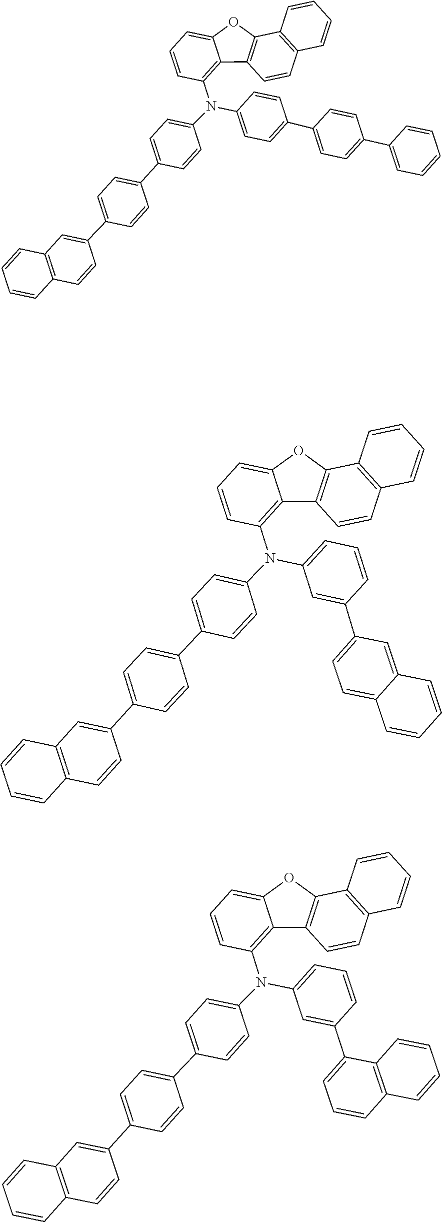 [SMILES: c1ccc(-c2ccc(-c3ccc(N(c4ccc(-c5ccc(-c6ccc7ccccc7c6)cc5)cc4)c4cccc5oc6c7ccccc7ccc6c45)cc3)cc2)cc1 ; c1cc(-c2ccc3ccccc3c2)cc(N(c2ccc(-c3ccc(-c4ccc5ccccc5c4)cc3)cc2)c2cccc3oc4c5ccccc5ccc4c23)c1 ; c1cc(-c2cccc3ccccc23)cc(N(c2ccc(-c3ccc(-c4ccc5ccccc5c4)cc3)cc2)c2cccc3oc4c5ccccc5ccc4c23)c1]

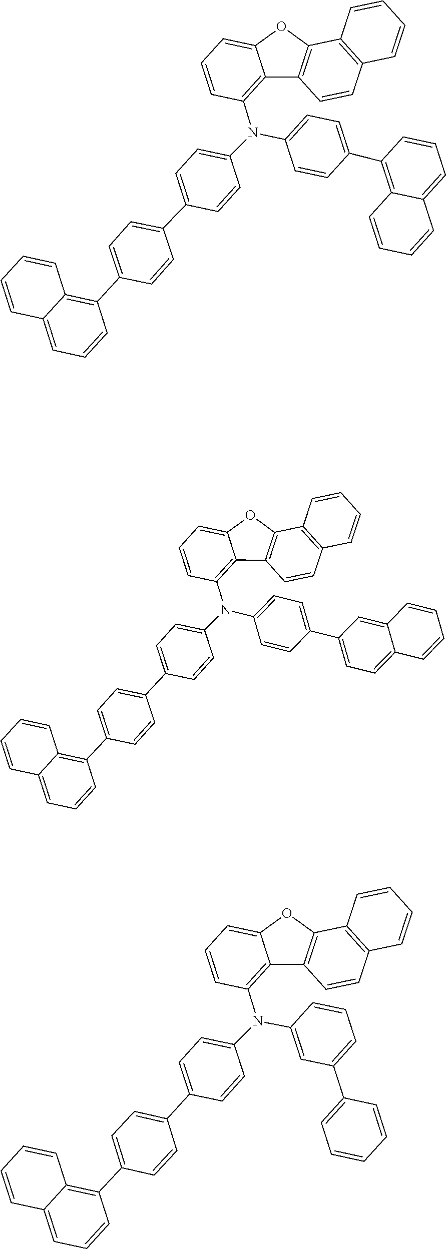 [SMILES: c1ccc2c(-c3ccc(-c4ccc(N(c5ccc(-c6cccc7ccccc67)cc5)c5cccc6oc7c8ccccc8ccc7c56)cc4)cc3)cccc2c1 ; c1ccc2cc(-c3ccc(N(c4ccc(-c5ccc(-c6cccc7ccccc67)cc5)cc4)c4cccc5oc6c7ccccc7ccc6c45)cc3)ccc2c1 ; c1ccc(-c2cccc(N(c3ccc(-c4ccc(-c5cccc6ccccc56)cc4)cc3)c3cccc4oc5c6ccccc6ccc5c34)c2)cc1]

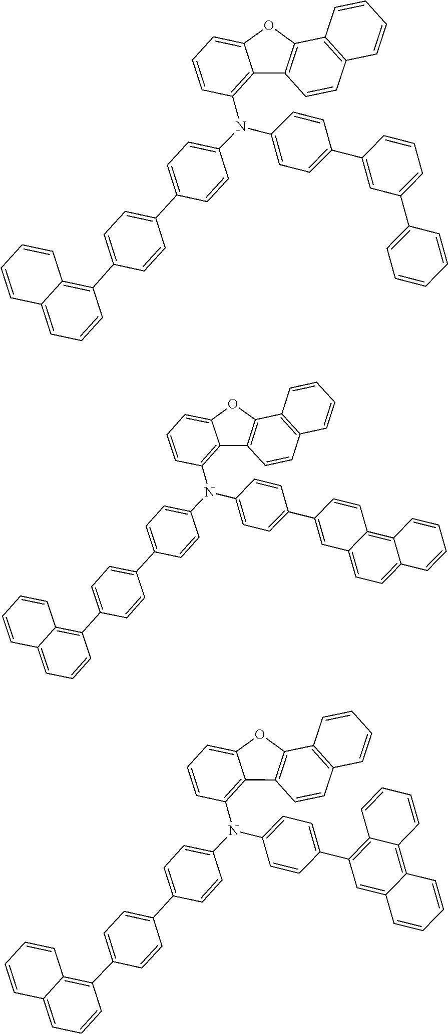 [SMILES: c1ccc(-c2cccc(-c3ccc(N(c4ccc(-c5ccc(-c6cccc7ccccc67)cc5)cc4)c4cccc5oc6c7ccccc7ccc6c45)cc3)c2)cc1 ; c1ccc2c(-c3ccc(-c4ccc(N(c5ccc(-c6ccc7c(ccc8ccccc87)c6)cc5)c5cccc6oc7c8ccccc8ccc7c56)cc4)cc3)cccc2c1 ; c1ccc2c(-c3ccc(-c4ccc(N(c5ccc(-c6cc7ccccc7c7ccccc67)cc5)c5cccc6oc7c8ccccc8ccc7c56)cc4)cc3)cccc2c1]

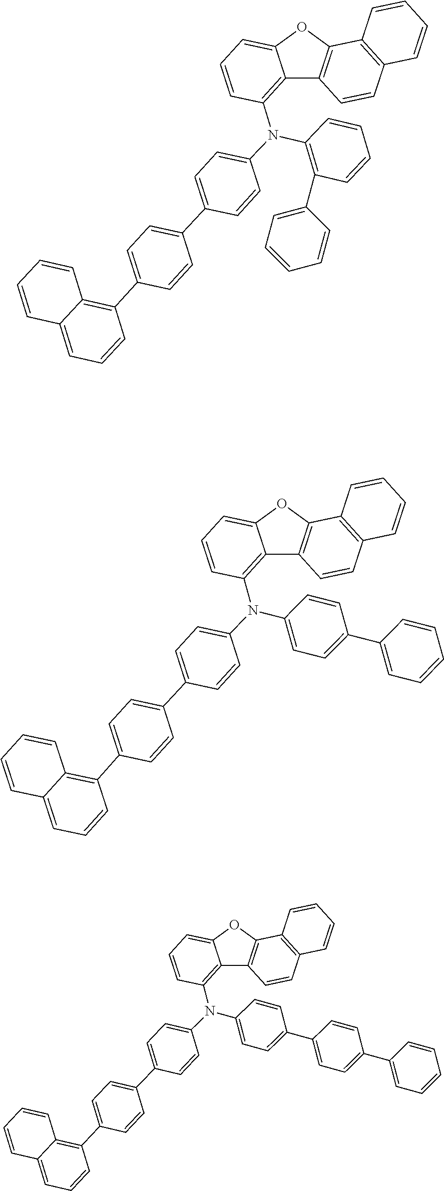 [SMILES: c1ccc(-c2ccccc2N(c2ccc(-c3ccc(-c4cccc5ccccc45)cc3)cc2)c2cccc3oc4c5ccccc5ccc4c23)cc1 ; c1ccc(-c2ccc(N(c3ccc(-c4ccc(-c5cccc6ccccc56)cc4)cc3)c3cccc4oc5c6ccccc6ccc5c34)cc2)cc1 ; c1ccc(-c2ccc(-c3ccc(N(c4ccc(-c5ccc(-c6cccc7ccccc67)cc5)cc4)c4cccc5oc6c7ccccc7ccc6c45)cc3)cc2)cc1]

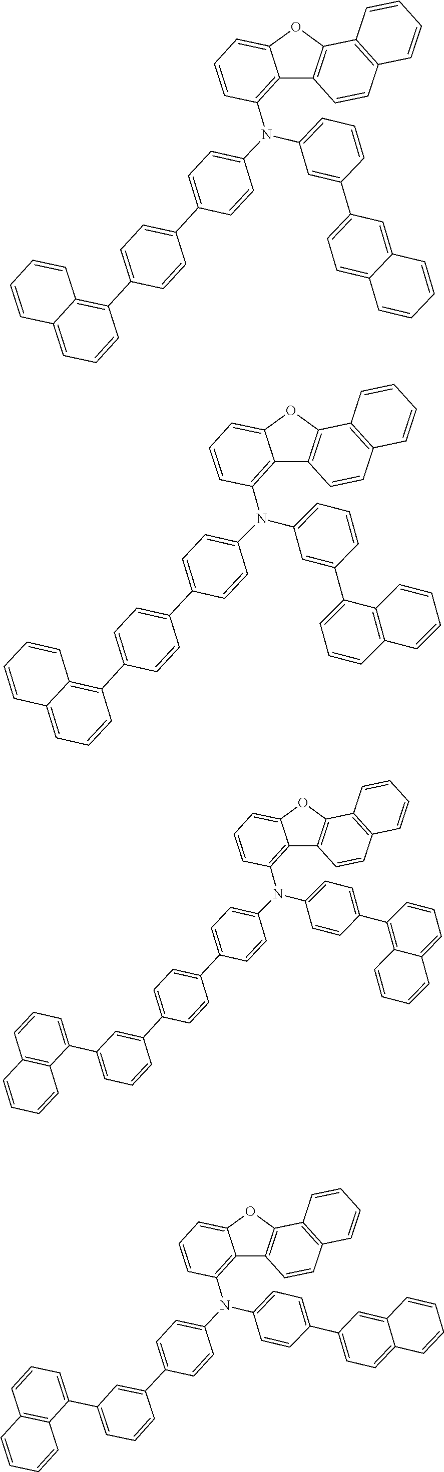 [SMILES: c1cc(-c2ccc3ccccc3c2)cc(N(c2ccc(-c3ccc(-c4cccc5ccccc45)cc3)cc2)c2cccc3oc4c5ccccc5ccc4c23)c1 ; c1cc(-c2cccc3ccccc23)cc(N(c2ccc(-c3ccc(-c4cccc5ccccc45)cc3)cc2)c2cccc3oc4c5ccccc5ccc4c23)c1 ; c1cc(-c2ccc(-c3ccc(N(c4ccc(-c5cccc6ccccc56)cc4)c4cccc5oc6c7ccccc7ccc6c45)cc3)cc2)cc(-c2cccc3ccccc23)c1 ; c1cc(-c2ccc(N(c3ccc(-c4ccc5ccccc5c4)cc3)c3cccc4oc5c6ccccc6ccc5c34)cc2)cc(-c2cccc3ccccc23)c1]

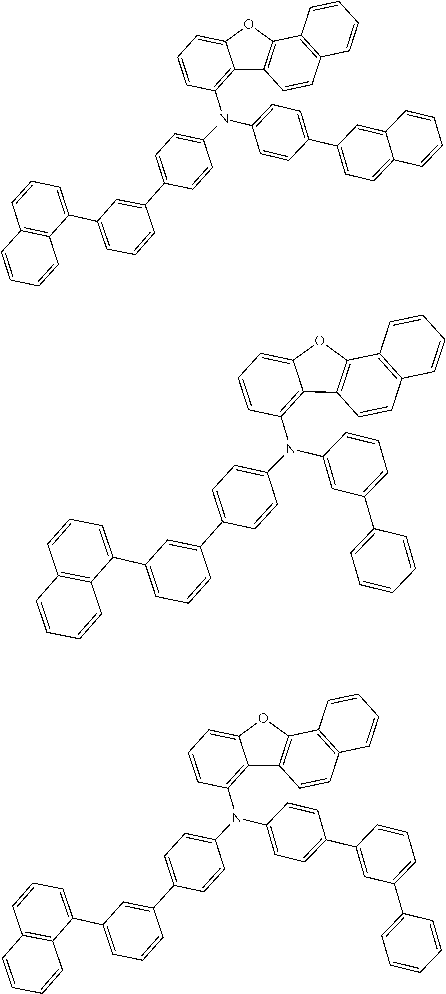 [SMILES: c1cc(-c2ccc(N(c3ccc(-c4ccc5ccccc5c4)cc3)c3cccc4oc5c6ccccc6ccc5c34)cc2)cc(-c2cccc3ccccc23)c1 ; c1ccc(-c2cccc(N(c3ccc(-c4cccc(-c5cccc6ccccc56)c4)cc3)c3cccc4oc5c6ccccc6ccc5c34)c2)cc1 ; c1ccc(-c2cccc(-c3ccc(N(c4ccc(-c5cccc(-c6cccc7ccccc67)c5)cc4)c4cccc5oc6c7ccccc7ccc6c45)cc3)c2)cc1]

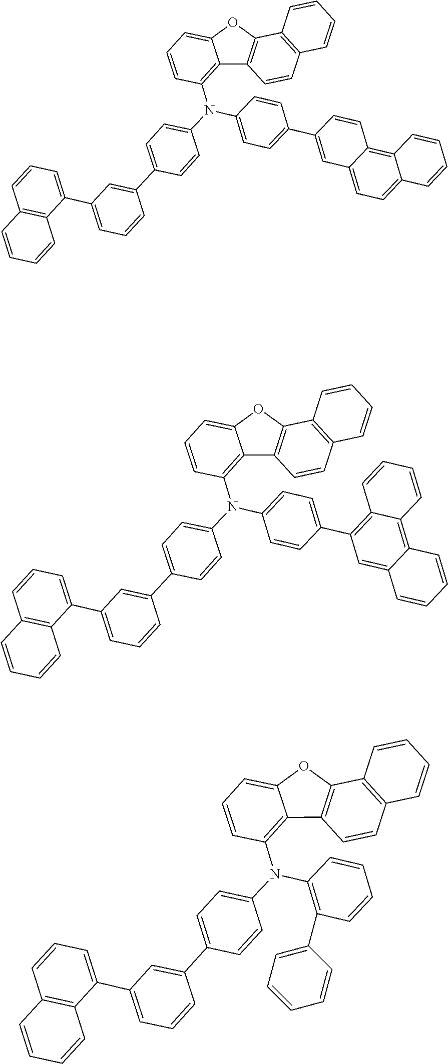 [SMILES: c1cc(-c2ccc(N(c3ccc(-c4ccc5c(ccc6ccccc65)c4)cc3)c3cccc4oc5c6ccccc6ccc5c34)cc2)cc(-c2cccc3ccccc23)c1 ; c1cc(-c2ccc(N(c3ccc(-c4cc5ccccc5c5ccccc45)cc3)c3cccc4oc5c6ccccc6ccc5c34)cc2)cc(-c2cccc3ccccc23)c1 ; c1ccc(-c2ccccc2N(c2ccc(-c3cccc(-c4cccc5ccccc45)c3)cc2)c2cccc3oc4c5ccccc5ccc4c23)cc1]

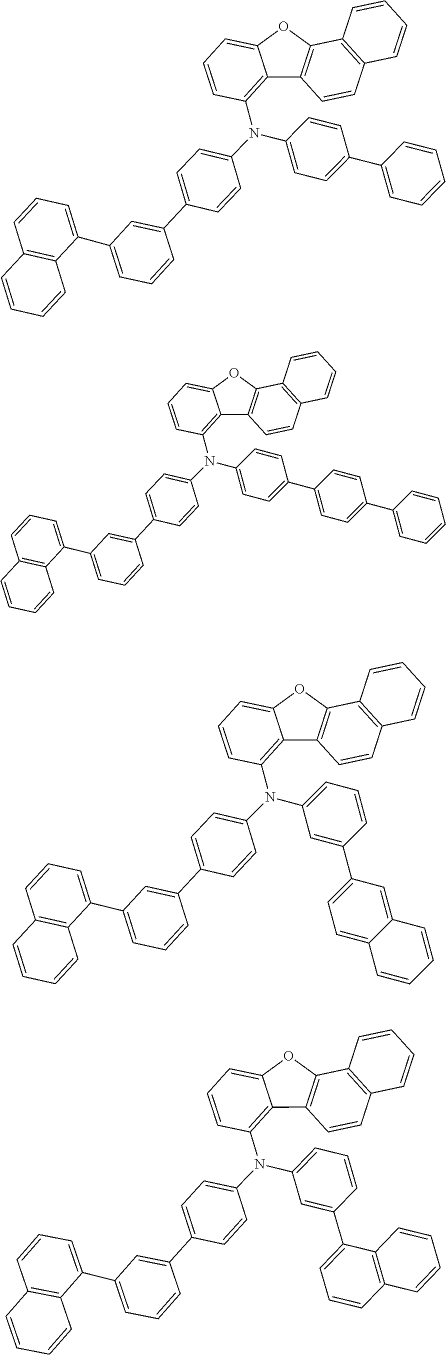 [SMILES: c1ccc(-c2ccc(N(c3ccc(-c4cccc(-c5cccc6ccccc56)c4)cc3)c3cccc4oc5c6ccccc6ccc5c34)cc2)cc1 ; c1ccc(-c2ccc(-c3ccc(N(c4ccc(-c5cccc(-c6cccc7ccccc67)c5)cc4)c4cccc5oc6c7ccccc7ccc6c45)cc3)cc2)cc1 ; c1cc(-c2ccc(N(c3cccc(-c4ccc5ccccc5c4)c3)c3cccc4oc5c6ccccc6ccc5c34)cc2)cc(-c2cccc3ccccc23)c1 ; c1cc(-c2ccc(N(c3cccc(-c4cccc5ccccc45)c3)c3cccc4oc5c6ccccc6ccc5c34)cc2)cc(-c2cccc3ccccc23)c1]

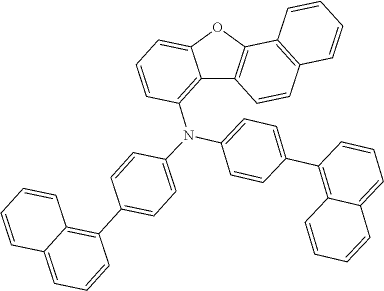 [SMILES: c1ccc2c(-c3ccc(N(c4ccc(-c5cccc6ccccc56)cc4)c4cccc5oc6c7ccccc7ccc6c45)cc3)cccc2c1]

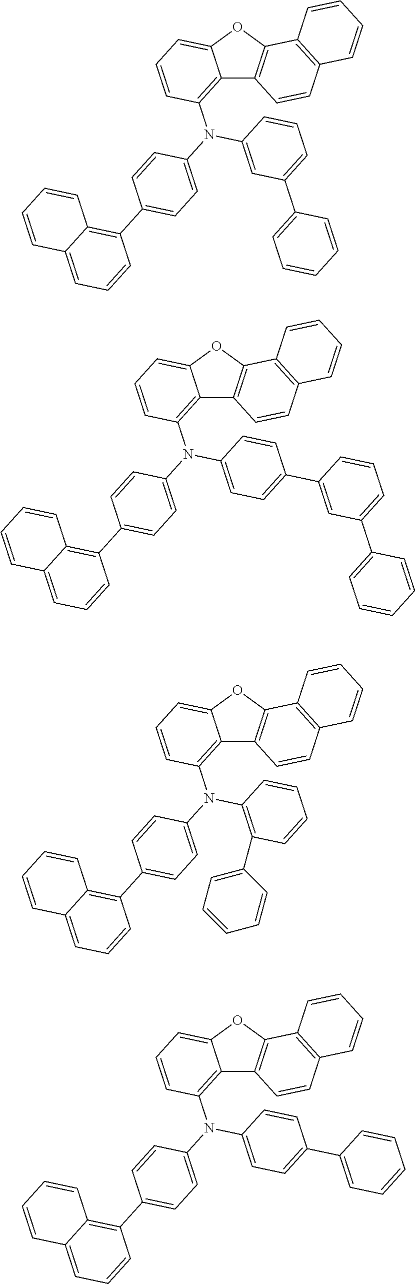

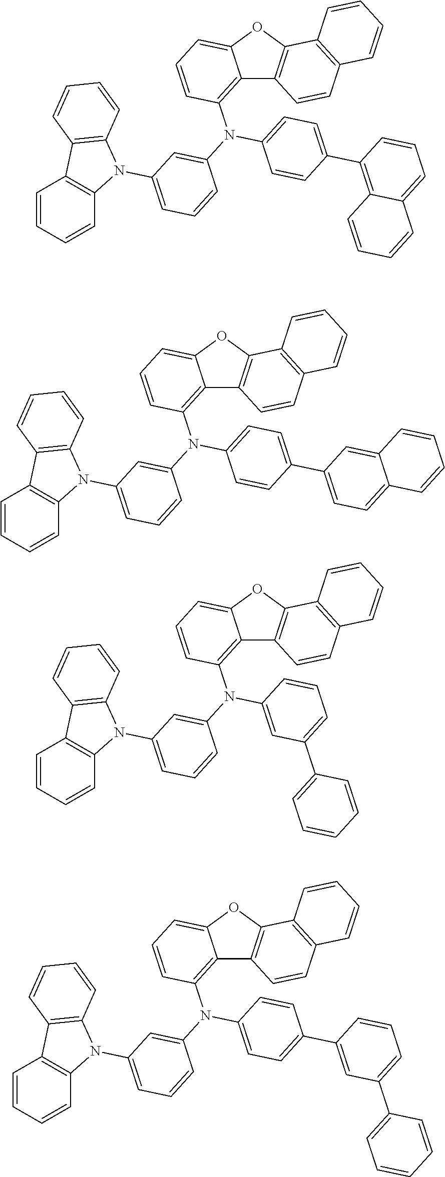

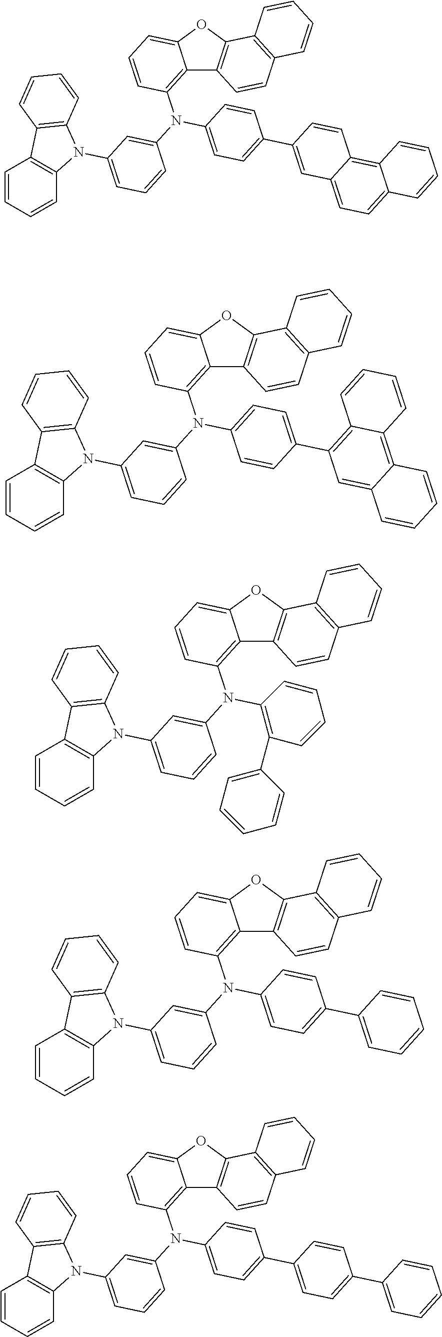 [SMILES: c1cc(N(c2ccc(-c3ccc4c(ccc5ccccc54)c3)cc2)c2cccc3oc4c5ccccc5ccc4c23)cc(-n2c3ccccc3c3ccccc32)c1 ; c1cc(N(c2ccc(-c3cc4ccccc4c4ccccc34)cc2)c2cccc3oc4c5ccccc5ccc4c23)cc(-n2c3ccccc3c3ccccc32)c1 ; c1ccc(-c2ccccc2N(c2cccc(-n3c4ccccc4c4ccccc43)c2)c2cccc3oc4c5ccccc5ccc4c23)cc1 ; c1ccc(-c2ccc(N(c3cccc(-n4c5ccccc5c5ccccc54)c3)c3cccc4oc5c6ccccc6ccc5c34)cc2)cc1 ; c1ccc(-c2ccc(-c3ccc(N(c4cccc(-n5c6ccccc6c6ccccc65)c4)c4cccc5oc6c7ccccc7ccc6c45)cc3)cc2)cc1]

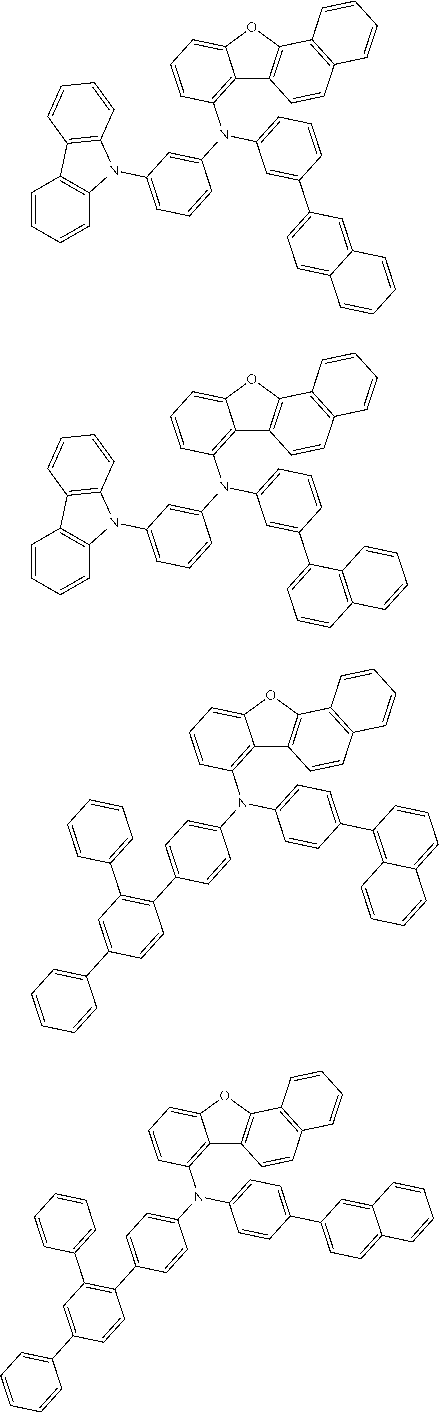

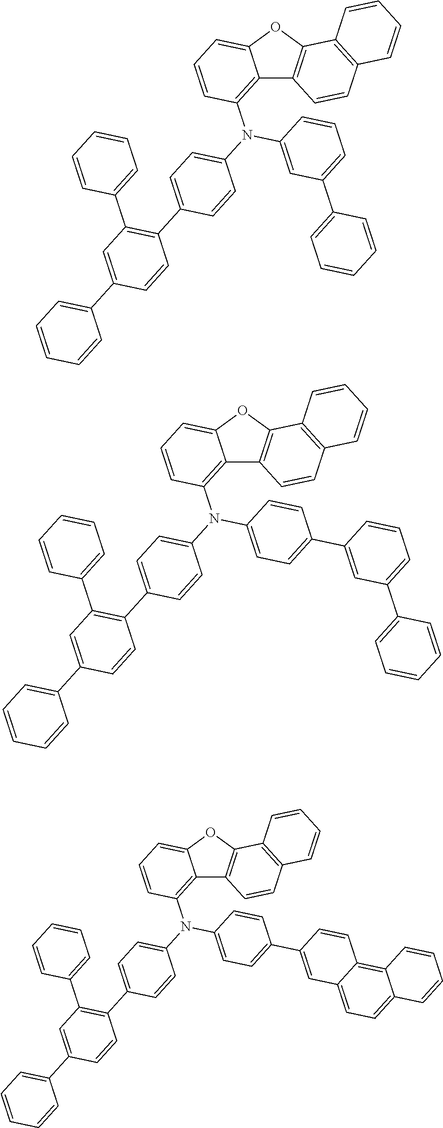 [SMILES: c1ccc(-c2cccc(N(c3ccc(-c4ccc(-c5ccccc5)cc4-c4ccccc4)cc3)c3cccc4oc5c6ccccc6ccc5c34)c2)cc1 ; c1ccc(-c2cccc(-c3ccc(N(c4ccc(-c5ccc(-c6ccccc6)cc5-c5ccccc5)cc4)c4cccc5oc6c7ccccc7ccc6c45)cc3)c2)cc1 ; c1ccc(-c2ccc(-c3ccc(N(c4ccc(-c5ccc6c(ccc7ccccc76)c5)cc4)c4cccc5oc6c7ccccc7ccc6c45)cc3)c(-c3ccccc3)c2)cc1]

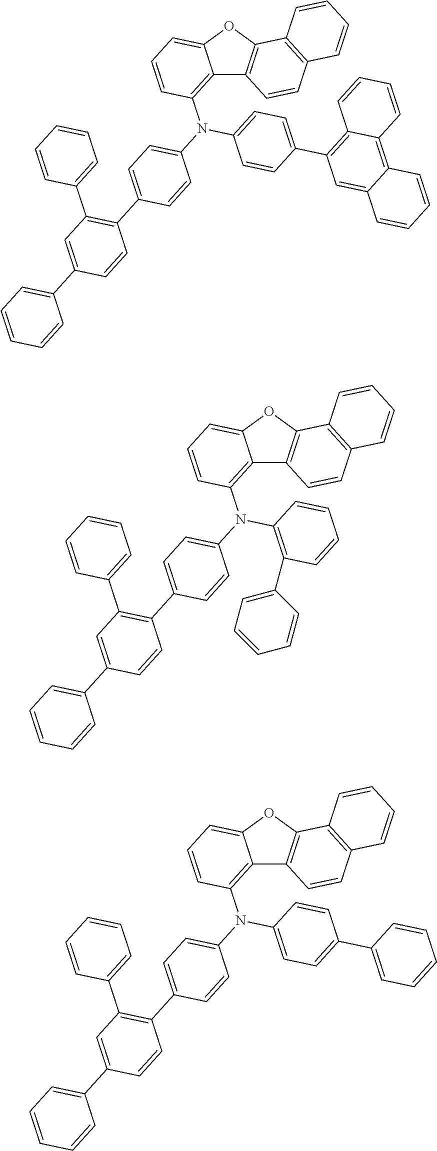 [SMILES: c1ccc(-c2ccc(-c3ccc(N(c4ccc(-c5cc6ccccc6c6ccccc56)cc4)c4cccc5oc6c7ccccc7ccc6c45)cc3)c(-c3ccccc3)c2)cc1 ; c1ccc(-c2ccc(-c3ccc(N(c4ccccc4-c4ccccc4)c4cccc5oc6c7ccccc7ccc6c45)cc3)c(-c3ccccc3)c2)cc1 ; c1ccc(-c2ccc(N(c3ccc(-c4ccc(-c5ccccc5)cc4-c4ccccc4)cc3)c3cccc4oc5c6ccccc6ccc5c34)cc2)cc1]

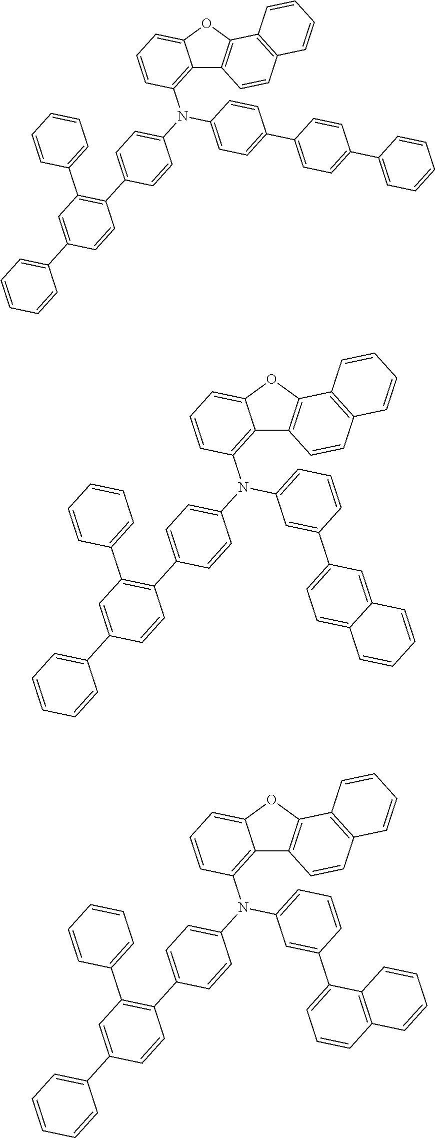 [SMILES: c1ccc(-c2ccc(-c3ccc(N(c4ccc(-c5ccc(-c6ccccc6)cc5-c5ccccc5)cc4)c4cccc5oc6c7ccccc7ccc6c45)cc3)cc2)cc1 ; c1ccc(-c2ccc(-c3ccc(N(c4cccc(-c5ccc6ccccc6c5)c4)c4cccc5oc6c7ccccc7ccc6c45)cc3)c(-c3ccccc3)c2)cc1 ; c1ccc(-c2ccc(-c3ccc(N(c4cccc(-c5cccc6ccccc56)c4)c4cccc5oc6c7ccccc7ccc6c45)cc3)c(-c3ccccc3)c2)cc1]

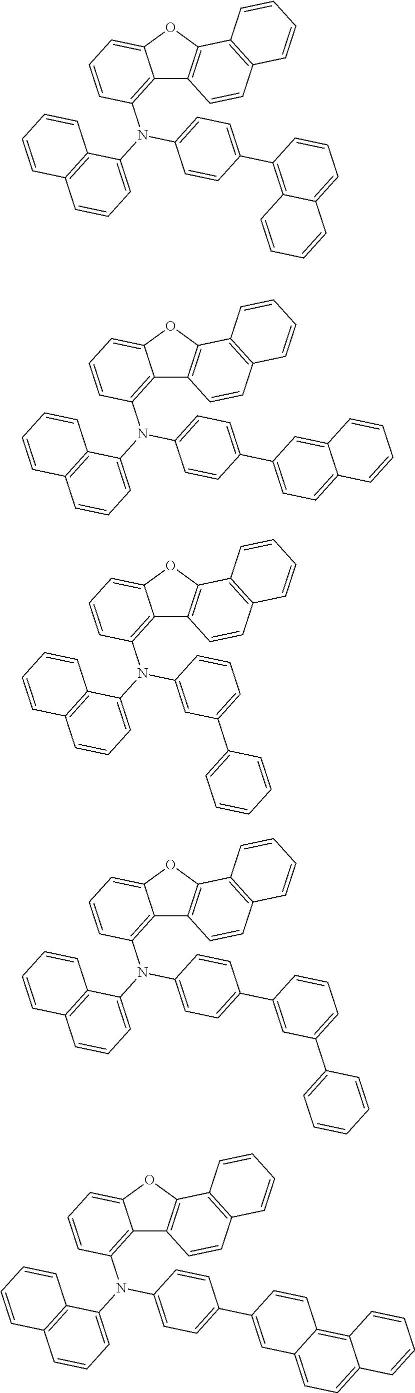

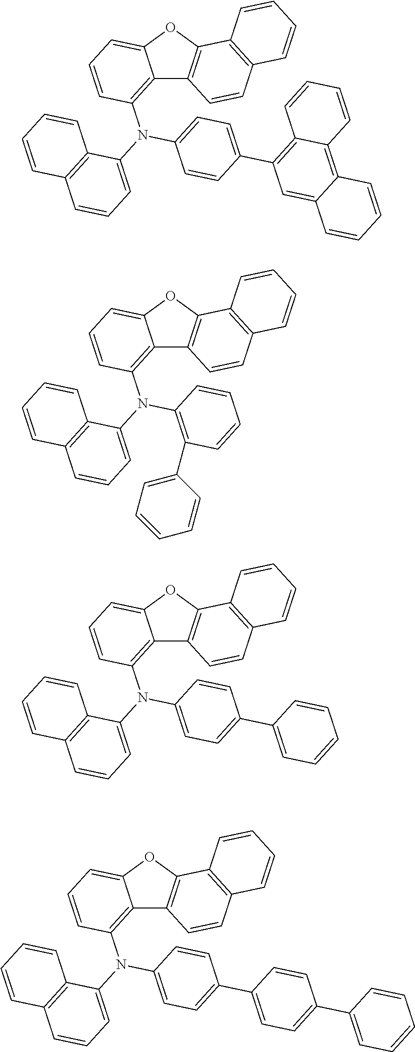 [SMILES: c1ccc2c(N(c3ccc(-c4cc5ccccc5c5ccccc45)cc3)c3cccc4oc5c6ccccc6ccc5c34)cccc2c1 ; c1ccc(-c2ccccc2N(c2cccc3ccccc23)c2cccc3oc4c5ccccc5ccc4c23)cc1 ; c1ccc(-c2ccc(N(c3cccc4ccccc34)c3cccc4oc5c6ccccc6ccc5c34)cc2)cc1 ; c1ccc(-c2ccc(-c3ccc(N(c4cccc5ccccc45)c4cccc5oc6c7ccccc7ccc6c45)cc3)cc2)cc1]

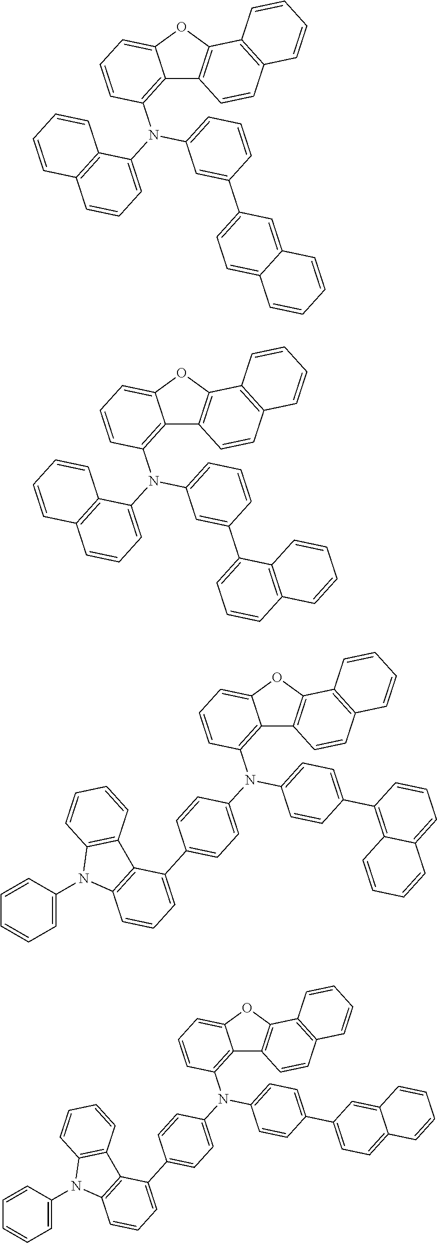

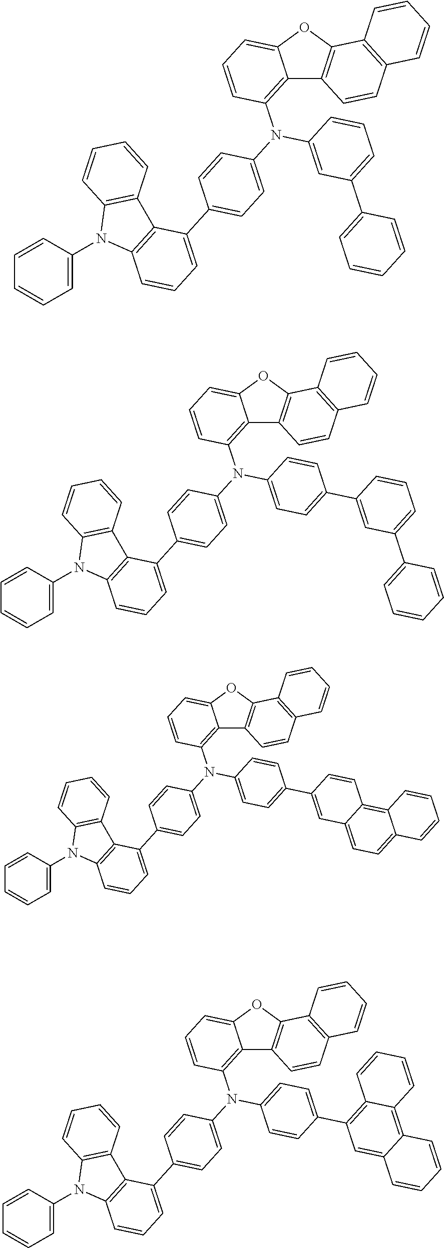 [SMILES: c1ccc(-c2cccc(N(c3ccc(-c4cccc5c4c4ccccc4n5-c4ccccc4)cc3)c3cccc4oc5c6ccccc6ccc5c34)c2)cc1 ; c1ccc(-c2cccc(-c3ccc(N(c4ccc(-c5cccc6c5c5ccccc5n6-c5ccccc5)cc4)c4cccc5oc6c7ccccc7ccc6c45)cc3)c2)cc1 ; c1ccc(-n2c3ccccc3c3c(-c4ccc(N(c5ccc(-c6ccc7c(ccc8ccccc87)c6)cc5)c5cccc6oc7c8ccccc8ccc7c56)cc4)cccc32)cc1 ; c1ccc(-n2c3ccccc3c3c(-c4ccc(N(c5ccc(-c6cc7ccccc7c7ccccc67)cc5)c5cccc6oc7c8ccccc8ccc7c56)cc4)cccc32)cc1]

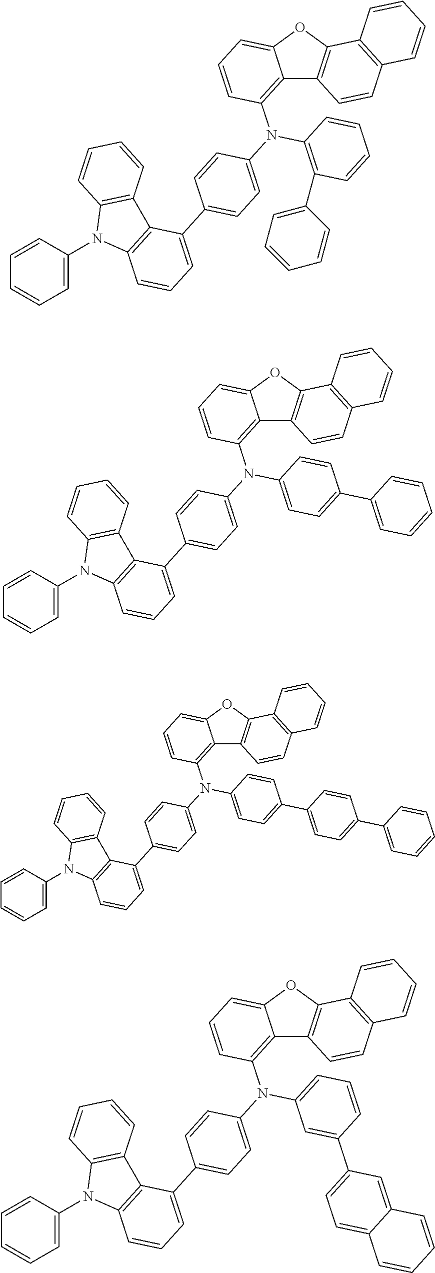 [SMILES: c1ccc(-c2ccccc2N(c2ccc(-c3cccc4c3c3ccccc3n4-c3ccccc3)cc2)c2cccc3oc4c5ccccc5ccc4c23)cc1 ; c1ccc(-c2ccc(N(c3ccc(-c4cccc5c4c4ccccc4n5-c4ccccc4)cc3)c3cccc4oc5c6ccccc6ccc5c34)cc2)cc1 ; c1ccc(-c2ccc(-c3ccc(N(c4ccc(-c5cccc6c5c5ccccc5n6-c5ccccc5)cc4)c4cccc5oc6c7ccccc7ccc6c45)cc3)cc2)cc1 ; c1ccc(-n2c3ccccc3c3c(-c4ccc(N(c5cccc(-c6ccc7ccccc7c6)c5)c5cccc6oc7c8ccccc8ccc7c56)cc4)cccc32)cc1]

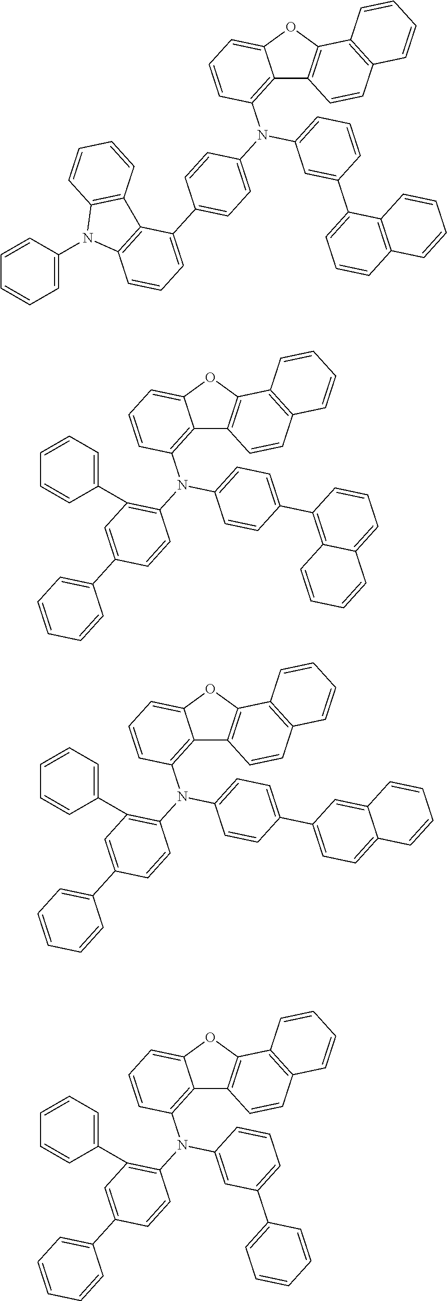 [SMILES: c1ccc(-n2c3ccccc3c3c(-c4ccc(N(c5cccc(-c6cccc7ccccc67)c5)c5cccc6oc7c8ccccc8ccc7c56)cc4)cccc32)cc1 ; c1ccc(-c2ccc(N(c3ccc(-c4cccc5ccccc45)cc3)c3cccc4oc5c6ccccc6ccc5c34)c(-c3ccccc3)c2)cc1 ; c1ccc(-c2ccc(N(c3ccc(-c4ccc5ccccc5c4)cc3)c3cccc4oc5c6ccccc6ccc5c34)c(-c3ccccc3)c2)cc1 ; c1ccc(-c2cccc(N(c3ccc(-c4ccccc4)cc3-c3ccccc3)c3cccc4oc5c6ccccc6ccc5c34)c2)cc1]

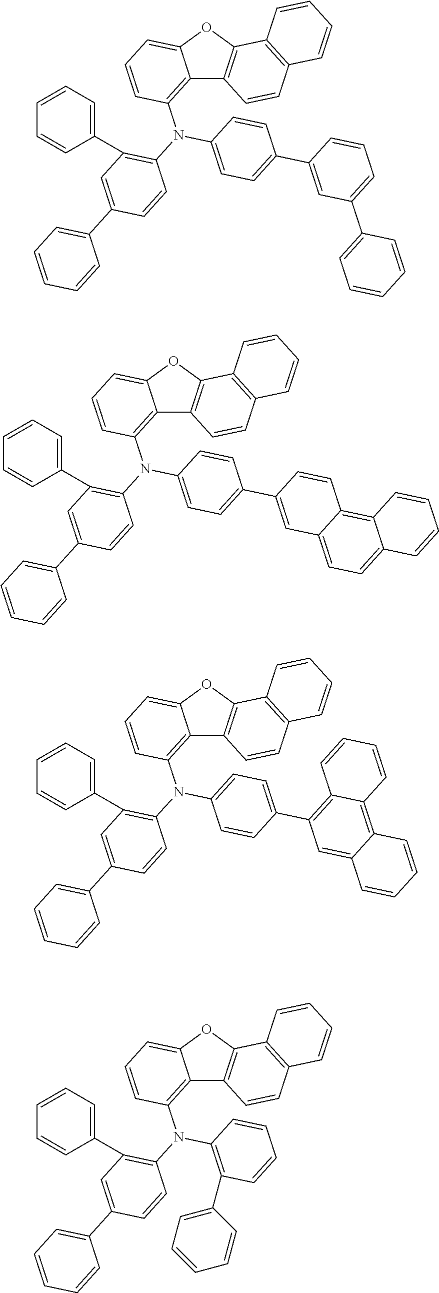 [SMILES: c1ccc(-c2cccc(-c3ccc(N(c4ccc(-c5ccccc5)cc4-c4ccccc4)c4cccc5oc6c7ccccc7ccc6c45)cc3)c2)cc1 ; c1ccc(-c2ccc(N(c3ccc(-c4ccc5c(ccc6ccccc65)c4)cc3)c3cccc4oc5c6ccccc6ccc5c34)c(-c3ccccc3)c2)cc1 ; c1ccc(-c2ccc(N(c3ccc(-c4cc5ccccc5c5ccccc45)cc3)c3cccc4oc5c6ccccc6ccc5c34)c(-c3ccccc3)c2)cc1 ; c1ccc(-c2ccc(N(c3ccccc3-c3ccccc3)c3cccc4oc5c6ccccc6ccc5c34)c(-c3ccccc3)c2)cc1]

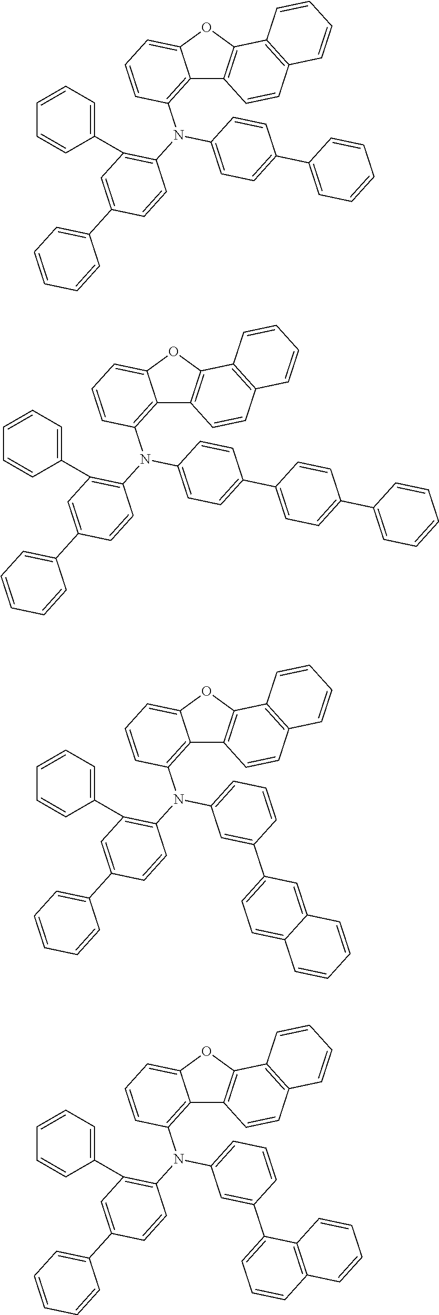 [SMILES: c1ccc(-c2ccc(N(c3ccc(-c4ccccc4)cc3-c3ccccc3)c3cccc4oc5c6ccccc6ccc5c34)cc2)cc1 ; c1ccc(-c2ccc(-c3ccc(N(c4ccc(-c5ccccc5)cc4-c4ccccc4)c4cccc5oc6c7ccccc7ccc6c45)cc3)cc2)cc1 ; c1ccc(-c2ccc(N(c3cccc(-c4ccc5ccccc5c4)c3)c3cccc4oc5c6ccccc6ccc5c34)c(-c3ccccc3)c2)cc1 ; c1ccc(-c2ccc(N(c3cccc(-c4cccc5ccccc45)c3)c3cccc4oc5c6ccccc6ccc5c34)c(-c3ccccc3)c2)cc1]

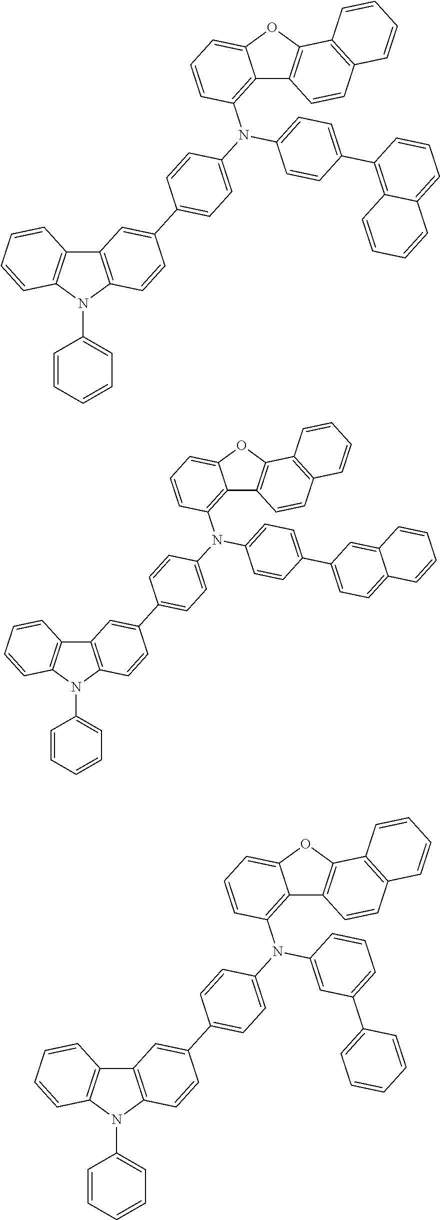 [SMILES: c1ccc(-n2c3ccccc3c3cc(-c4ccc(N(c5ccc(-c6cccc7ccccc67)cc5)c5cccc6oc7c8ccccc8ccc7c56)cc4)ccc32)cc1 ; c1ccc(-n2c3ccccc3c3cc(-c4ccc(N(c5ccc(-c6ccc7ccccc7c6)cc5)c5cccc6oc7c8ccccc8ccc7c56)cc4)ccc32)cc1 ; c1ccc(-c2cccc(N(c3ccc(-c4ccc5c(c4)c4ccccc4n5-c4ccccc4)cc3)c3cccc4oc5c6ccccc6ccc5c34)c2)cc1]

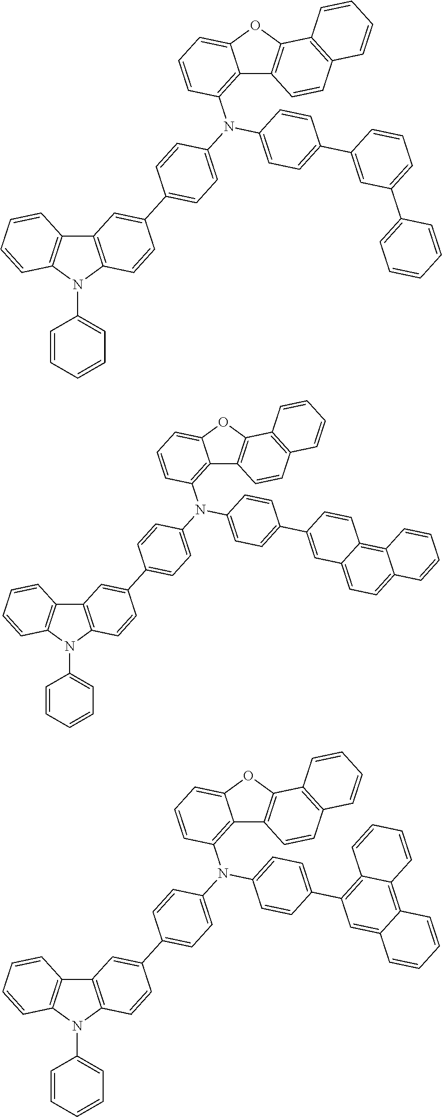 [SMILES: c1ccc(-c2cccc(-c3ccc(N(c4ccc(-c5ccc6c(c5)c5ccccc5n6-c5ccccc5)cc4)c4cccc5oc6c7ccccc7ccc6c45)cc3)c2)cc1 ; c1ccc(-n2c3ccccc3c3cc(-c4ccc(N(c5ccc(-c6ccc7c(ccc8ccccc87)c6)cc5)c5cccc6oc7c8ccccc8ccc7c56)cc4)ccc32)cc1 ; c1ccc(-n2c3ccccc3c3cc(-c4ccc(N(c5ccc(-c6cc7ccccc7c7ccccc67)cc5)c5cccc6oc7c8ccccc8ccc7c56)cc4)ccc32)cc1]

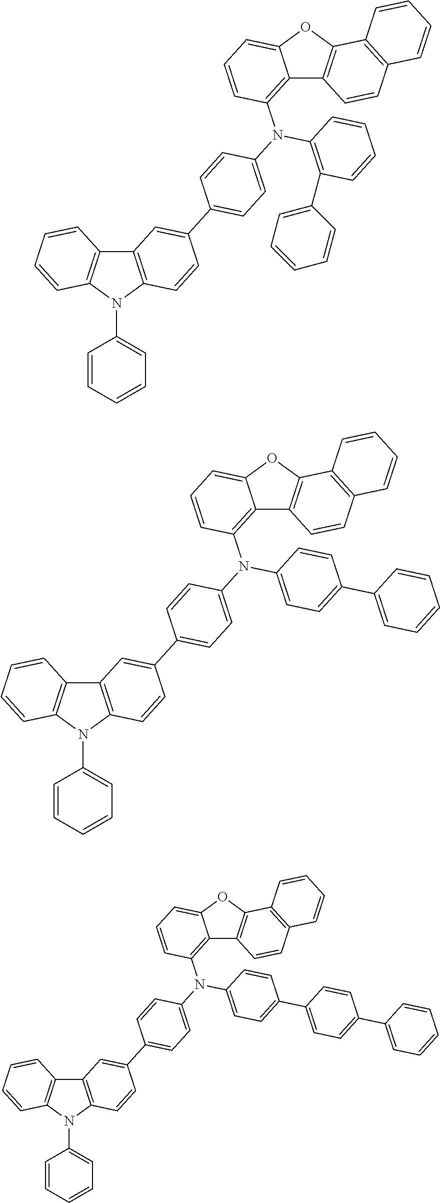 [SMILES: c1ccc(-c2ccccc2N(c2ccc(-c3ccc4c(c3)c3ccccc3n4-c3ccccc3)cc2)c2cccc3oc4c5ccccc5ccc4c23)cc1 ; c1ccc(-c2ccc(N(c3ccc(-c4ccc5c(c4)c4ccccc4n5-c4ccccc4)cc3)c3cccc4oc5c6ccccc6ccc5c34)cc2)cc1 ; c1ccc(-c2ccc(-c3ccc(N(c4ccc(-c5ccc6c(c5)c5ccccc5n6-c5ccccc5)cc4)c4cccc5oc6c7ccccc7ccc6c45)cc3)cc2)cc1]

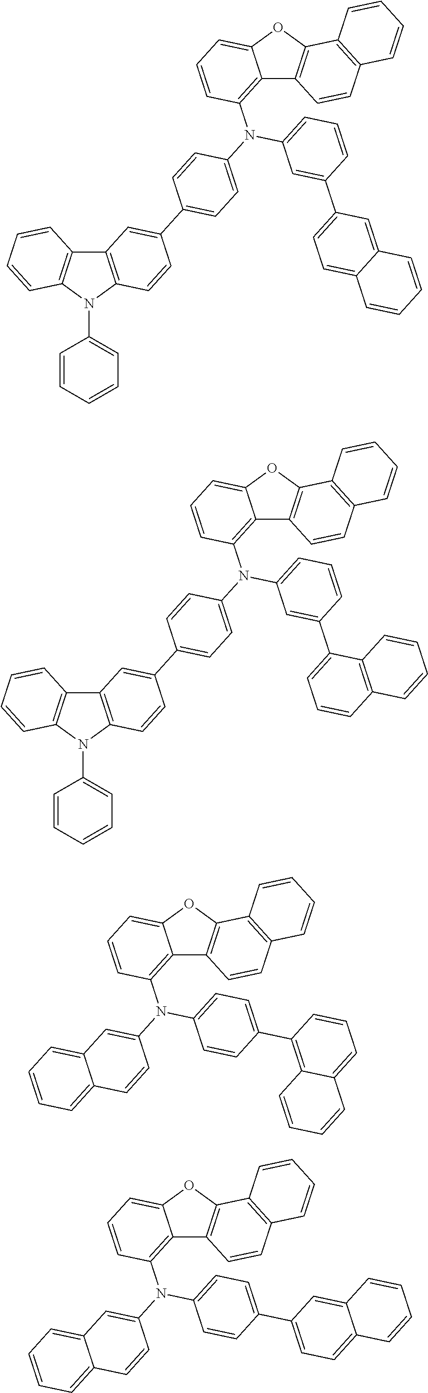

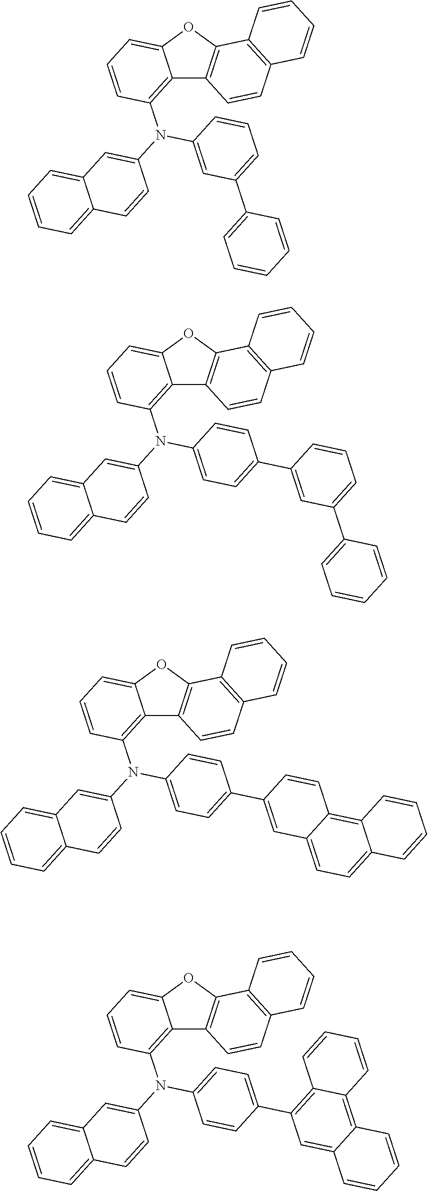

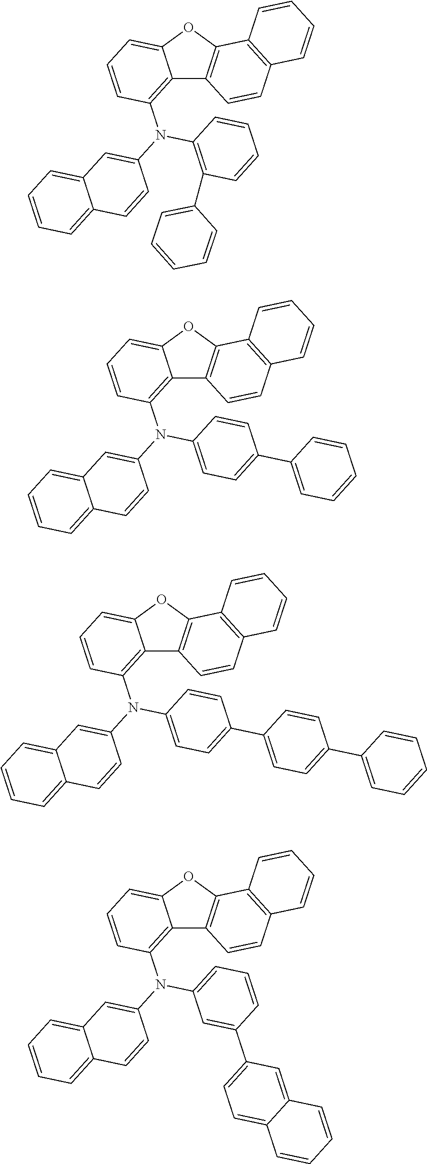 [SMILES: c1ccc(-c2ccccc2N(c2ccc3ccccc3c2)c2cccc3oc4c5ccccc5ccc4c23)cc1 ; c1ccc(-c2ccc(N(c3ccc4ccccc4c3)c3cccc4oc5c6ccccc6ccc5c34)cc2)cc1 ; c1ccc(-c2ccc(-c3ccc(N(c4ccc5ccccc5c4)c4cccc5oc6c7ccccc7ccc6c45)cc3)cc2)cc1 ; c1cc(-c2ccc3ccccc3c2)cc(N(c2ccc3ccccc3c2)c2cccc3oc4c5ccccc5ccc4c23)c1]

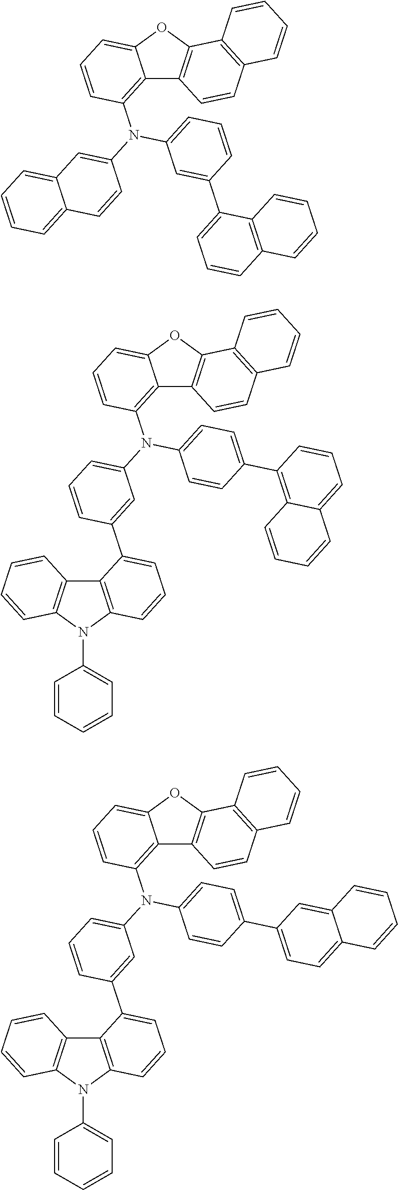

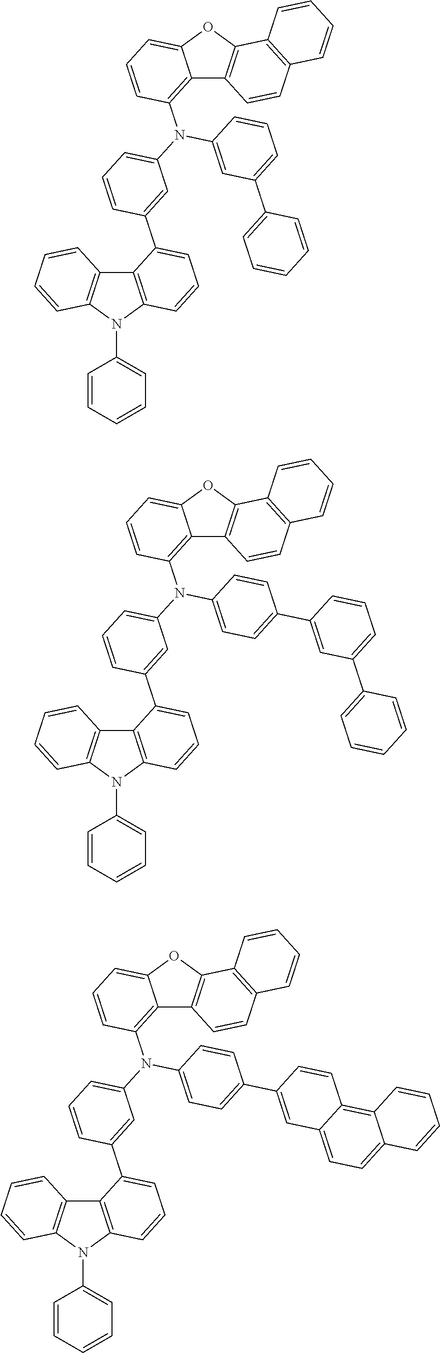 [SMILES: c1ccc(-c2cccc(N(c3cccc(-c4cccc5c4c4ccccc4n5-c4ccccc4)c3)c3cccc4oc5c6ccccc6ccc5c34)c2)cc1 ; c1ccc(-c2cccc(-c3ccc(N(c4cccc(-c5cccc6c5c5ccccc5n6-c5ccccc5)c4)c4cccc5oc6c7ccccc7ccc6c45)cc3)c2)cc1 ; c1ccc(-n2c3ccccc3c3c(-c4cccc(N(c5ccc(-c6ccc7c(ccc8ccccc87)c6)cc5)c5cccc6oc7c8ccccc8ccc7c56)c4)cccc32)cc1]

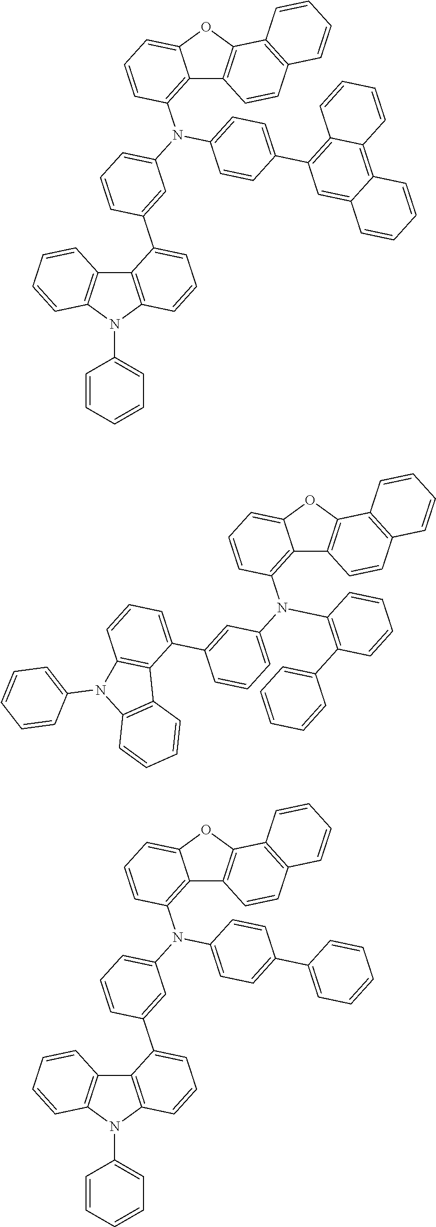 [SMILES: c1ccc(-n2c3ccccc3c3c(-c4cccc(N(c5ccc(-c6cc7ccccc7c7ccccc67)cc5)c5cccc6oc7c8ccccc8ccc7c56)c4)cccc32)cc1 ; c1ccc(-c2ccccc2N(c2cccc(-c3cccc4c3c3ccccc3n4-c3ccccc3)c2)c2cccc3oc4c5ccccc5ccc4c23)cc1 ; c1ccc(-c2ccc(N(c3cccc(-c4cccc5c4c4ccccc4n5-c4ccccc4)c3)c3cccc4oc5c6ccccc6ccc5c34)cc2)cc1]

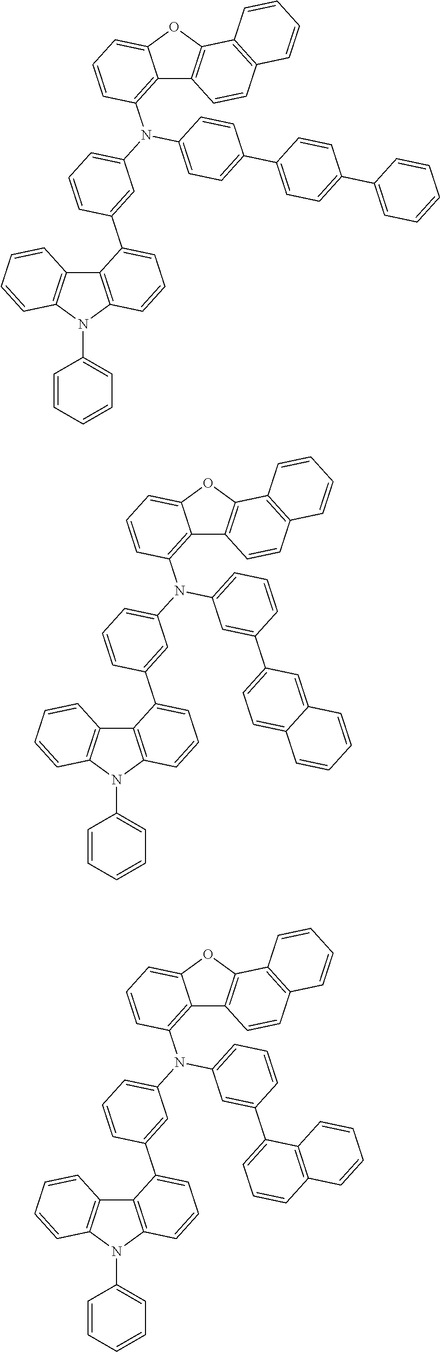 [SMILES: c1ccc(-c2ccc(-c3ccc(N(c4cccc(-c5cccc6c5c5ccccc5n6-c5ccccc5)c4)c4cccc5oc6c7ccccc7ccc6c45)cc3)cc2)cc1 ; c1ccc(-n2c3ccccc3c3c(-c4cccc(N(c5cccc(-c6ccc7ccccc7c6)c5)c5cccc6oc7c8ccccc8ccc7c56)c4)cccc32)cc1 ; c1ccc(-n2c3ccccc3c3c(-c4cccc(N(c5cccc(-c6cccc7ccccc67)c5)c5cccc6oc7c8ccccc8ccc7c56)c4)cccc32)cc1]

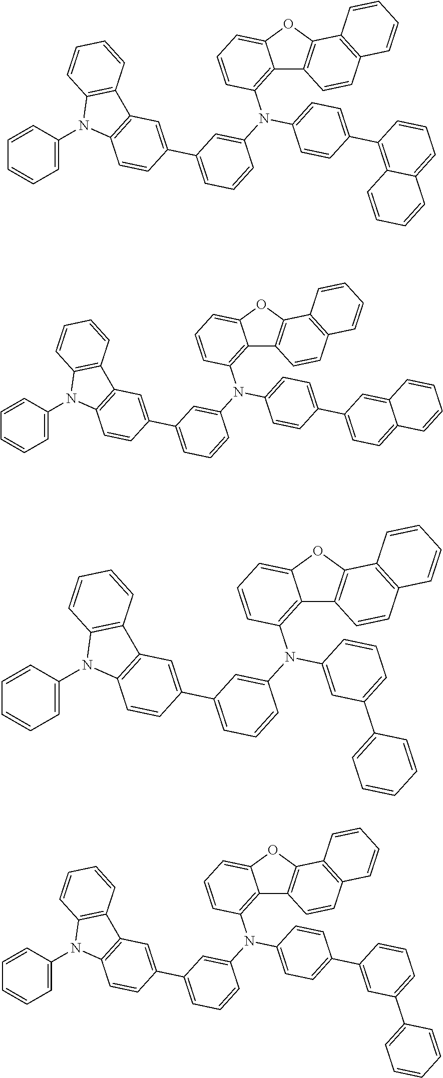 [SMILES: c1ccc(-n2c3ccccc3c3cc(-c4cccc(N(c5ccc(-c6cccc7ccccc67)cc5)c5cccc6oc7c8ccccc8ccc7c56)c4)ccc32)cc1 ; c1ccc(-n2c3ccccc3c3cc(-c4cccc(N(c5ccc(-c6ccc7ccccc7c6)cc5)c5cccc6oc7c8ccccc8ccc7c56)c4)ccc32)cc1 ; c1ccc(-c2cccc(N(c3cccc(-c4ccc5c(c4)c4ccccc4n5-c4ccccc4)c3)c3cccc4oc5c6ccccc6ccc5c34)c2)cc1 ; c1ccc(-c2cccc(-c3ccc(N(c4cccc(-c5ccc6c(c5)c5ccccc5n6-c5ccccc5)c4)c4cccc5oc6c7ccccc7ccc6c45)cc3)c2)cc1]

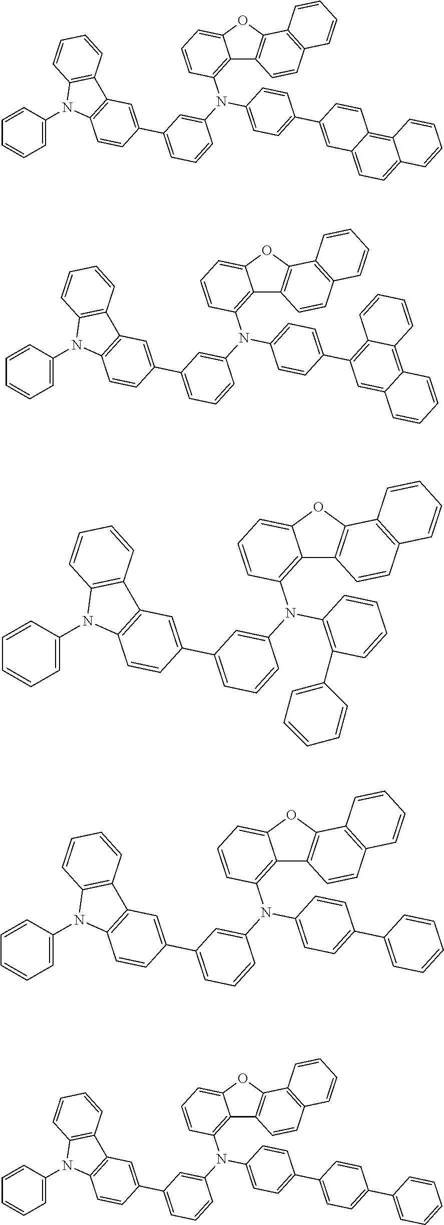 [SMILES: c1ccc(-n2c3ccccc3c3cc(-c4cccc(N(c5ccc(-c6ccc7c(ccc8ccccc87)c6)cc5)c5cccc6oc7c8ccccc8ccc7c56)c4)ccc32)cc1 ; c1ccc(-n2c3ccccc3c3cc(-c4cccc(N(c5ccc(-c6cc7ccccc7c7ccccc67)cc5)c5cccc6oc7c8ccccc8ccc7c56)c4)ccc32)cc1 ; c1ccc(-c2ccccc2N(c2cccc(-c3ccc4c(c3)c3ccccc3n4-c3ccccc3)c2)c2cccc3oc4c5ccccc5ccc4c23)cc1 ; c1ccc(-c2ccc(N(c3cccc(-c4ccc5c(c4)c4ccccc4n5-c4ccccc4)c3)c3cccc4oc5c6ccccc6ccc5c34)cc2)cc1 ; c1ccc(-c2ccc(-c3ccc(N(c4cccc(-c5ccc6c(c5)c5ccccc5n6-c5ccccc5)c4)c4cccc5oc6c7ccccc7ccc6c45)cc3)cc2)cc1]

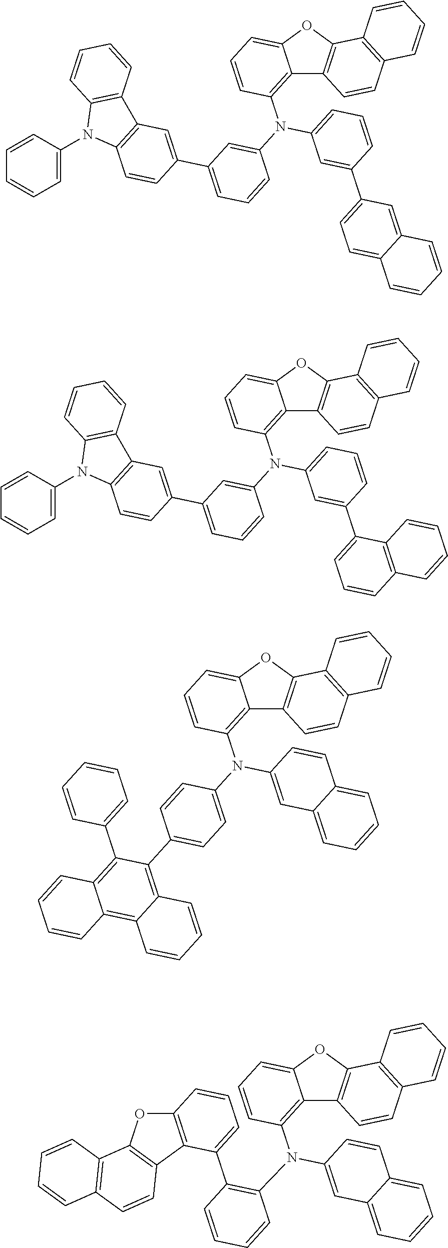

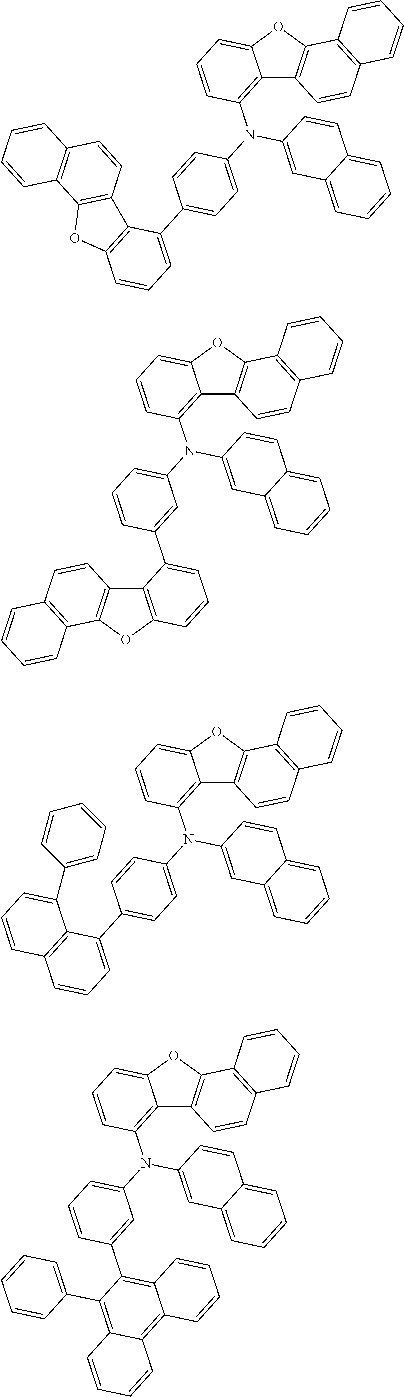 [SMILES: c1ccc2cc(N(c3ccc(-c4cccc5oc6c7ccccc7ccc6c45)cc3)c3cccc4oc5c6ccccc6ccc5c34)ccc2c1 ; c1cc(-c2cccc3oc4c5ccccc5ccc4c23)cc(N(c2ccc3ccccc3c2)c2cccc3oc4c5ccccc5ccc4c23)c1 ; c1ccc(-c2cccc3cccc(-c4ccc(N(c5ccc6ccccc6c5)c5cccc6oc7c8ccccc8ccc7c56)cc4)c23)cc1 ; c1ccc(-c2c(-c3cccc(N(c4ccc5ccccc5c4)c4cccc5oc6c7ccccc7ccc6c45)c3)c3ccccc3c3ccccc23)cc1]

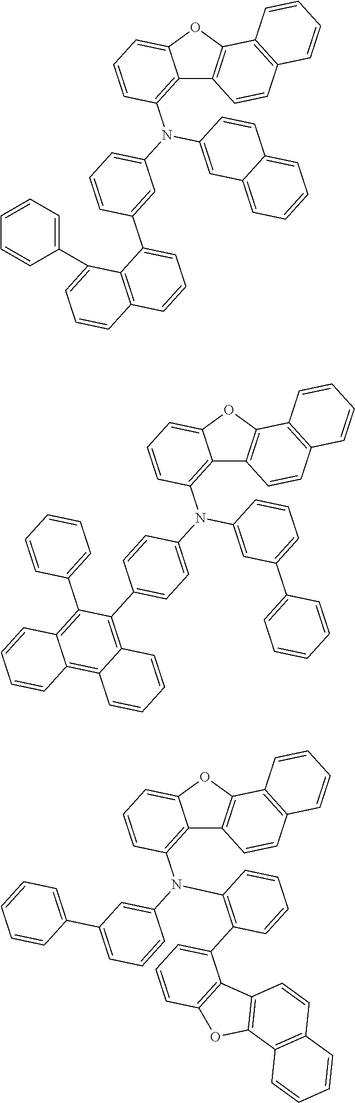 [SMILES: c1ccc(-c2cccc3cccc(-c4cccc(N(c5ccc6ccccc6c5)c5cccc6oc7c8ccccc8ccc7c56)c4)c23)cc1 ; c1ccc(-c2cccc(N(c3ccc(-c4c(-c5ccccc5)c5ccccc5c5ccccc45)cc3)c3cccc4oc5c6ccccc6ccc5c34)c2)cc1 ; c1ccc(-c2cccc(N(c3ccccc3-c3cccc4oc5c6ccccc6ccc5c34)c3cccc4oc5c6ccccc6ccc5c34)c2)cc1]

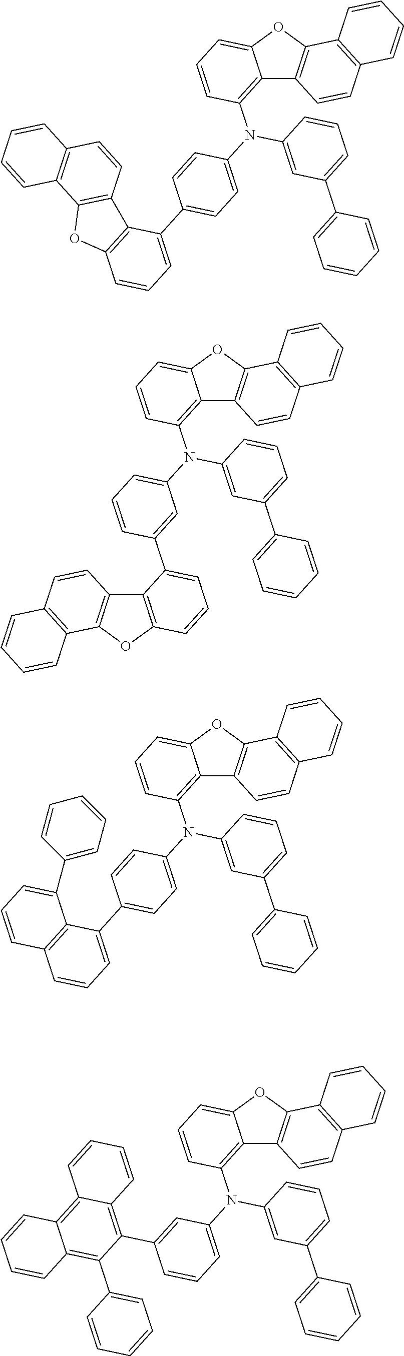 [SMILES: c1ccc(-c2cccc(N(c3ccc(-c4cccc5oc6c7ccccc7ccc6c45)cc3)c3cccc4oc5c6ccccc6ccc5c34)c2)cc1 ; c1ccc(-c2cccc(N(c3cccc(-c4cccc5oc6c7ccccc7ccc6c45)c3)c3cccc4oc5c6ccccc6ccc5c34)c2)cc1 ; c1ccc(-c2cccc(N(c3ccc(-c4cccc5cccc(-c6ccccc6)c45)cc3)c3cccc4oc5c6ccccc6ccc5c34)c2)cc1 ; c1ccc(-c2cccc(N(c3cccc(-c4c(-c5ccccc5)c5ccccc5c5ccccc45)c3)c3cccc4oc5c6ccccc6ccc5c34)c2)cc1]

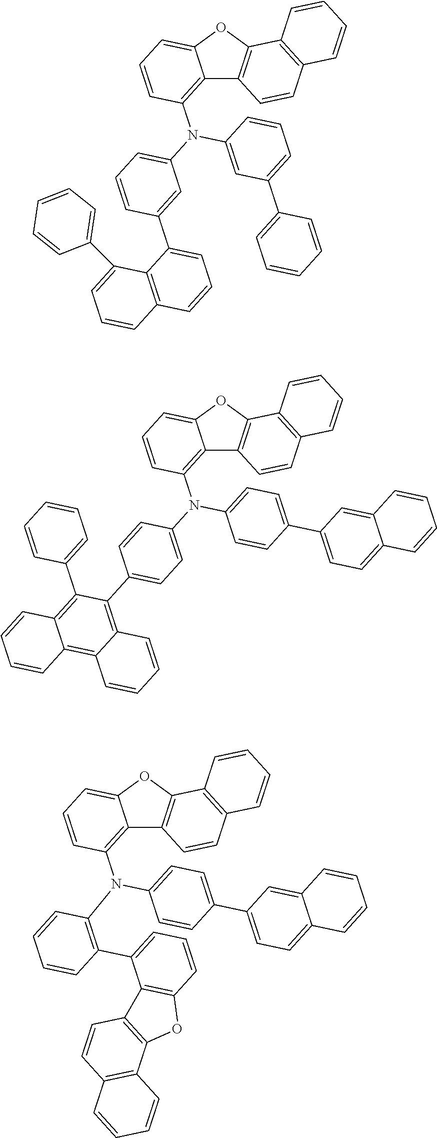 [SMILES: c1ccc(-c2cccc(N(c3cccc(-c4cccc5cccc(-c6ccccc6)c45)c3)c3cccc4oc5c6ccccc6ccc5c34)c2)cc1 ; c1ccc(-c2c(-c3ccc(N(c4ccc(-c5ccc6ccccc6c5)cc4)c4cccc5oc6c7ccccc7ccc6c45)cc3)c3ccccc3c3ccccc23)cc1 ; c1ccc(N(c2ccc(-c3ccc4ccccc4c3)cc2)c2cccc3oc4c5ccccc5ccc4c23)c(-c2cccc3oc4c5ccccc5ccc4c23)c1]

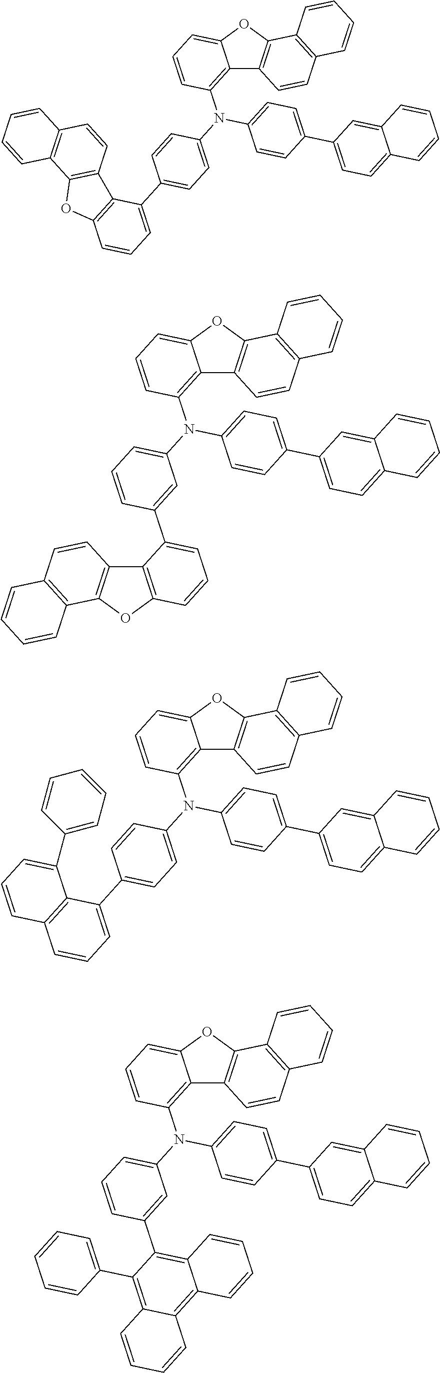 [SMILES: c1ccc2cc(-c3ccc(N(c4ccc(-c5cccc6oc7c8ccccc8ccc7c56)cc4)c4cccc5oc6c7ccccc7ccc6c45)cc3)ccc2c1 ; c1cc(-c2cccc3oc4c5ccccc5ccc4c23)cc(N(c2ccc(-c3ccc4ccccc4c3)cc2)c2cccc3oc4c5ccccc5ccc4c23)c1 ; c1ccc(-c2cccc3cccc(-c4ccc(N(c5ccc(-c6ccc7ccccc7c6)cc5)c5cccc6oc7c8ccccc8ccc7c56)cc4)c23)cc1 ; c1ccc(-c2c(-c3cccc(N(c4ccc(-c5ccc6ccccc6c5)cc4)c4cccc5oc6c7ccccc7ccc6c45)c3)c3ccccc3c3ccccc23)cc1]

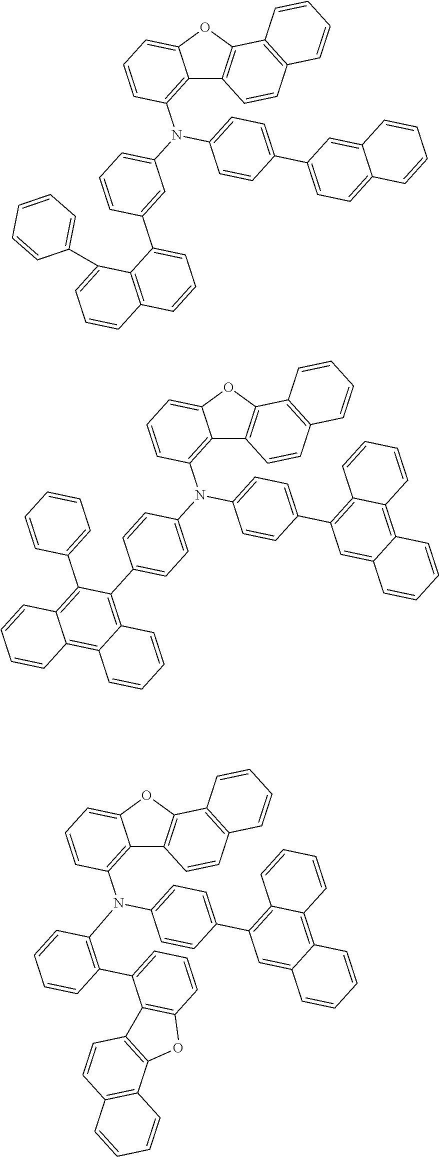 [SMILES: c1ccc(-c2cccc3cccc(-c4cccc(N(c5ccc(-c6ccc7ccccc7c6)cc5)c5cccc6oc7c8ccccc8ccc7c56)c4)c23)cc1 ; c1ccc(-c2c(-c3ccc(N(c4ccc(-c5cc6ccccc6c6ccccc56)cc4)c4cccc5oc6c7ccccc7ccc6c45)cc3)c3ccccc3c3ccccc23)cc1 ; c1ccc(N(c2ccc(-c3cc4ccccc4c4ccccc34)cc2)c2cccc3oc4c5ccccc5ccc4c23)c(-c2cccc3oc4c5ccccc5ccc4c23)c1]

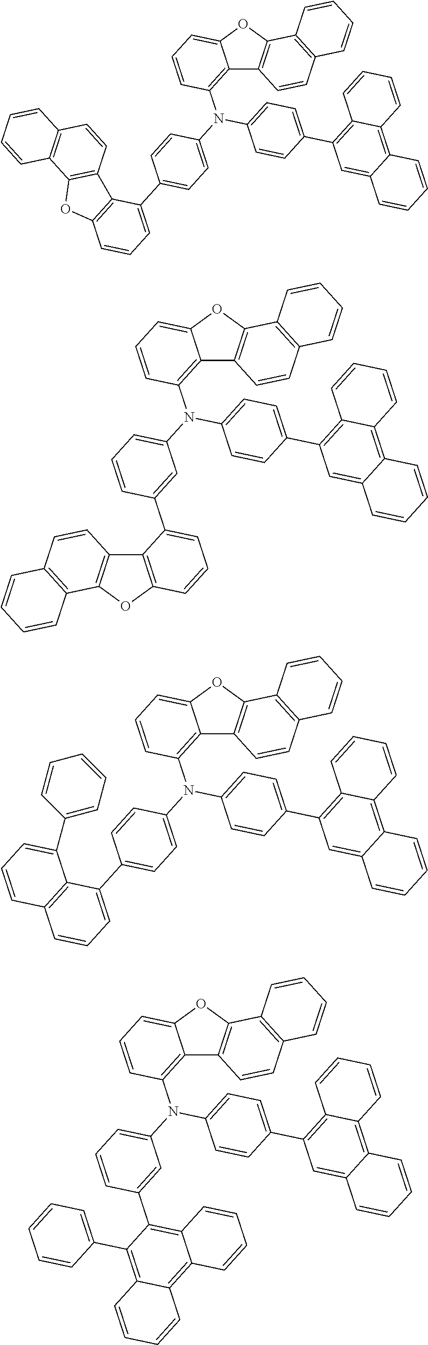 [SMILES: c1ccc2c(c1)cc(-c1ccc(N(c3ccc(-c4cccc5oc6c7ccccc7ccc6c45)cc3)c3cccc4oc5c6ccccc6ccc5c34)cc1)c1ccccc12 ; c1cc(-c2cccc3oc4c5ccccc5ccc4c23)cc(N(c2ccc(-c3cc4ccccc4c4ccccc34)cc2)c2cccc3oc4c5ccccc5ccc4c23)c1 ; c1ccc(-c2cccc3cccc(-c4ccc(N(c5ccc(-c6cc7ccccc7c7ccccc67)cc5)c5cccc6oc7c8ccccc8ccc7c56)cc4)c23)cc1 ; c1ccc(-c2c(-c3cccc(N(c4ccc(-c5cc6ccccc6c6ccccc56)cc4)c4cccc5oc6c7ccccc7ccc6c45)c3)c3ccccc3c3ccccc23)cc1]

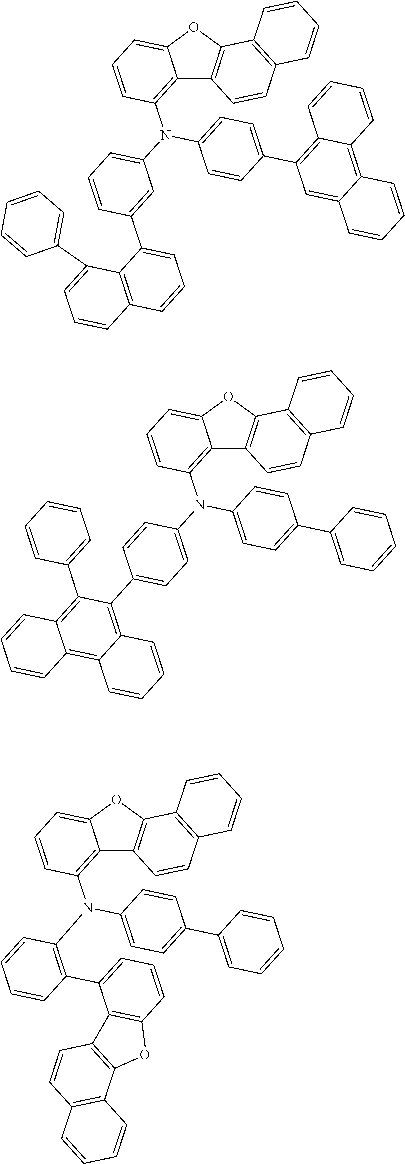 [SMILES: c1ccc(-c2cccc3cccc(-c4cccc(N(c5ccc(-c6cc7ccccc7c7ccccc67)cc5)c5cccc6oc7c8ccccc8ccc7c56)c4)c23)cc1 ; c1ccc(-c2ccc(N(c3ccc(-c4c(-c5ccccc5)c5ccccc5c5ccccc45)cc3)c3cccc4oc5c6ccccc6ccc5c34)cc2)cc1 ; c1ccc(-c2ccc(N(c3ccccc3-c3cccc4oc5c6ccccc6ccc5c34)c3cccc4oc5c6ccccc6ccc5c34)cc2)cc1]

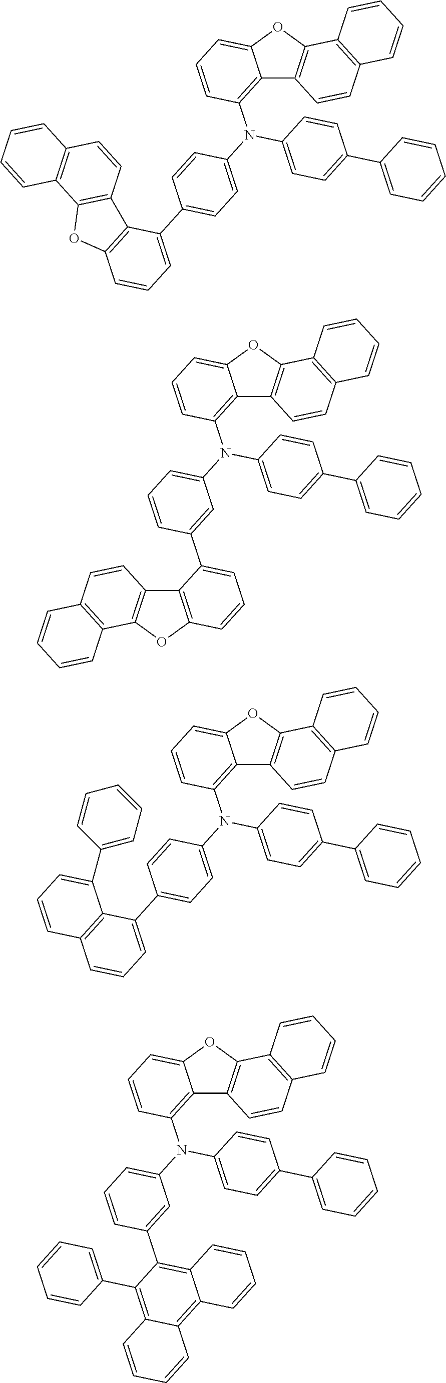 [SMILES: c1ccc(-c2ccc(N(c3ccc(-c4cccc5oc6c7ccccc7ccc6c45)cc3)c3cccc4oc5c6ccccc6ccc5c34)cc2)cc1 ; c1ccc(-c2ccc(N(c3cccc(-c4cccc5oc6c7ccccc7ccc6c45)c3)c3cccc4oc5c6ccccc6ccc5c34)cc2)cc1 ; c1ccc(-c2ccc(N(c3ccc(-c4cccc5cccc(-c6ccccc6)c45)cc3)c3cccc4oc5c6ccccc6ccc5c34)cc2)cc1 ; c1ccc(-c2ccc(N(c3cccc(-c4c(-c5ccccc5)c5ccccc5c5ccccc45)c3)c3cccc4oc5c6ccccc6ccc5c34)cc2)cc1]

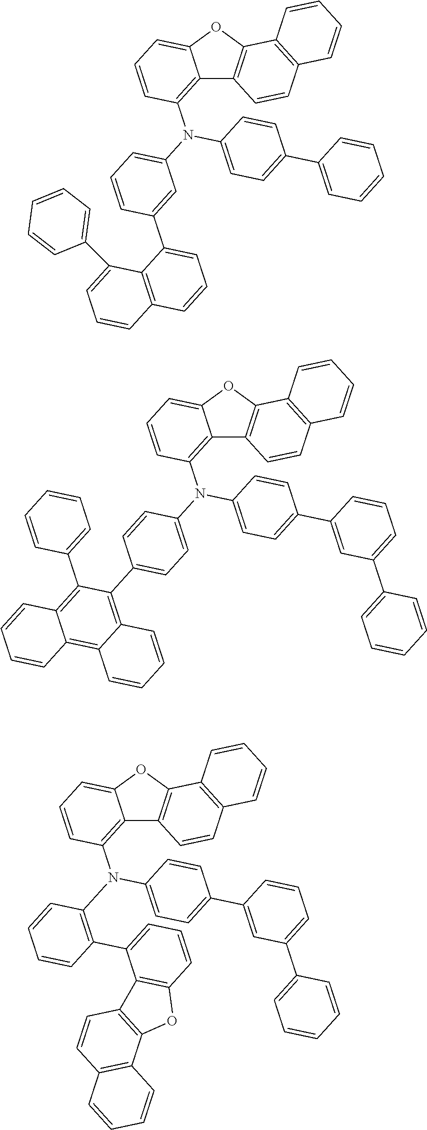 [SMILES: c1ccc(-c2ccc(N(c3cccc(-c4cccc5cccc(-c6ccccc6)c45)c3)c3cccc4oc5c6ccccc6ccc5c34)cc2)cc1 ; c1ccc(-c2cccc(-c3ccc(N(c4ccc(-c5c(-c6ccccc6)c6ccccc6c6ccccc56)cc4)c4cccc5oc6c7ccccc7ccc6c45)cc3)c2)cc1 ; c1ccc(-c2cccc(-c3ccc(N(c4ccccc4-c4cccc5oc6c7ccccc7ccc6c45)c4cccc5oc6c7ccccc7ccc6c45)cc3)c2)cc1]

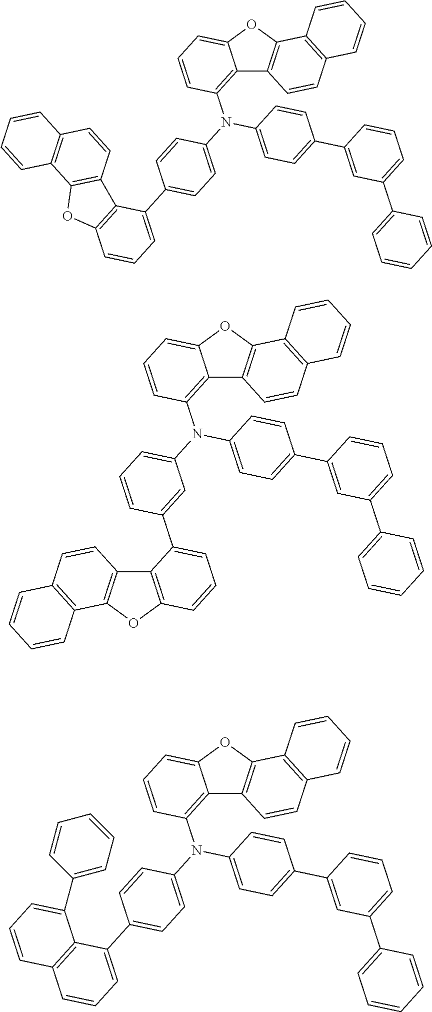 [SMILES: c1ccc(-c2cccc(-c3ccc(N(c4ccc(-c5cccc6oc7c8ccccc8ccc7c56)cc4)c4cccc5oc6c7ccccc7ccc6c45)cc3)c2)cc1 ; c1ccc(-c2cccc(-c3ccc(N(c4cccc(-c5cccc6oc7c8ccccc8ccc7c56)c4)c4cccc5oc6c7ccccc7ccc6c45)cc3)c2)cc1 ; c1ccc(-c2cccc(-c3ccc(N(c4ccc(-c5cccc6cccc(-c7ccccc7)c56)cc4)c4cccc5oc6c7ccccc7ccc6c45)cc3)c2)cc1]

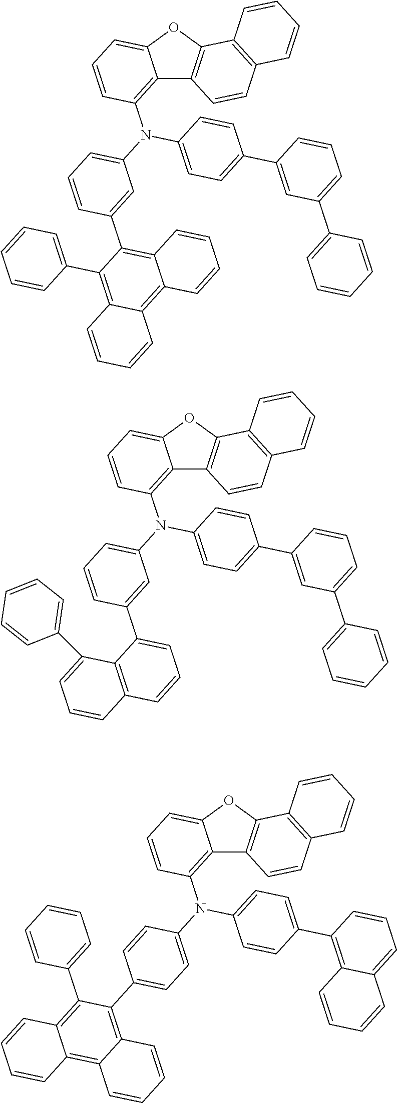 [SMILES: c1ccc(-c2cccc(-c3ccc(N(c4cccc(-c5c(-c6ccccc6)c6ccccc6c6ccccc56)c4)c4cccc5oc6c7ccccc7ccc6c45)cc3)c2)cc1 ; c1ccc(-c2cccc(-c3ccc(N(c4cccc(-c5cccc6cccc(-c7ccccc7)c56)c4)c4cccc5oc6c7ccccc7ccc6c45)cc3)c2)cc1 ; c1ccc(-c2c(-c3ccc(N(c4ccc(-c5cccc6ccccc56)cc4)c4cccc5oc6c7ccccc7ccc6c45)cc3)c3ccccc3c3ccccc23)cc1]

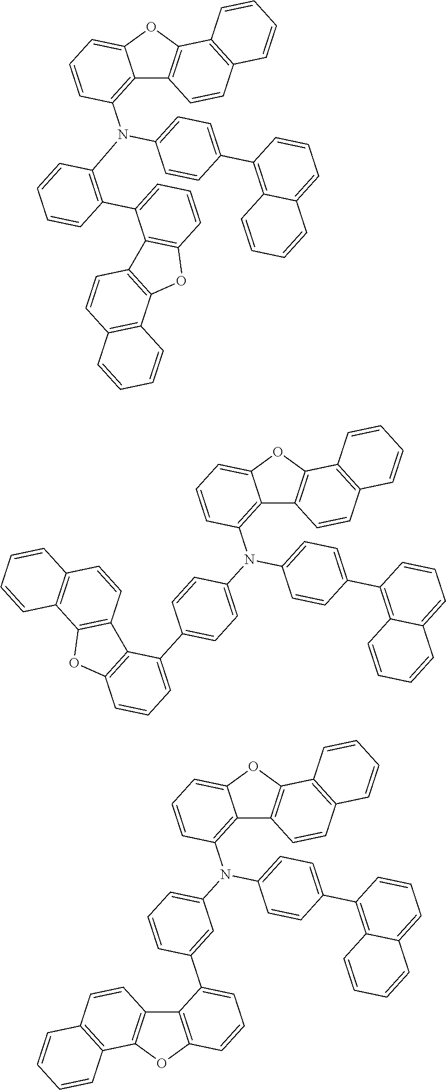 [SMILES: c1ccc(N(c2ccc(-c3cccc4ccccc34)cc2)c2cccc3oc4c5ccccc5ccc4c23)c(-c2cccc3oc4c5ccccc5ccc4c23)c1 ; c1ccc2c(-c3ccc(N(c4ccc(-c5cccc6oc7c8ccccc8ccc7c56)cc4)c4cccc5oc6c7ccccc7ccc6c45)cc3)cccc2c1 ; c1cc(-c2cccc3oc4c5ccccc5ccc4c23)cc(N(c2ccc(-c3cccc4ccccc34)cc2)c2cccc3oc4c5ccccc5ccc4c23)c1]

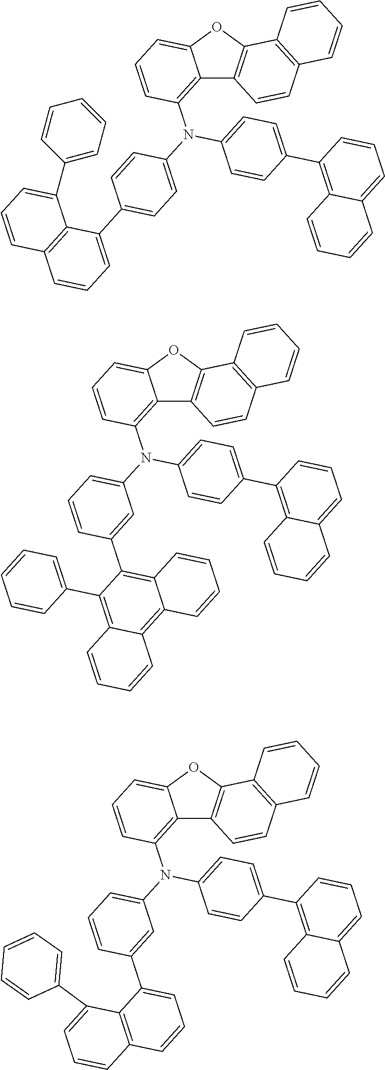 [SMILES: c1ccc(-c2cccc3cccc(-c4ccc(N(c5ccc(-c6cccc7ccccc67)cc5)c5cccc6oc7c8ccccc8ccc7c56)cc4)c23)cc1 ; c1ccc(-c2c(-c3cccc(N(c4ccc(-c5cccc6ccccc56)cc4)c4cccc5oc6c7ccccc7ccc6c45)c3)c3ccccc3c3ccccc23)cc1 ; c1ccc(-c2cccc3cccc(-c4cccc(N(c5ccc(-c6cccc7ccccc67)cc5)c5cccc6oc7c8ccccc8ccc7c56)c4)c23)cc1]

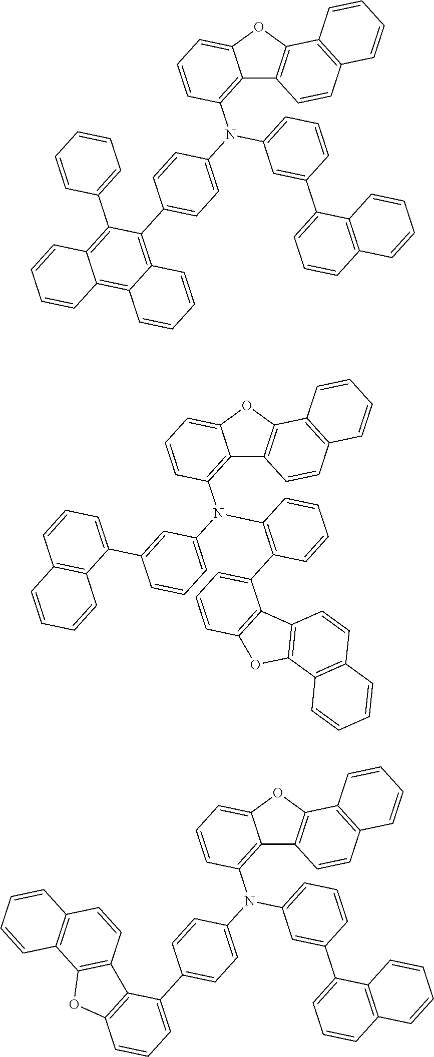

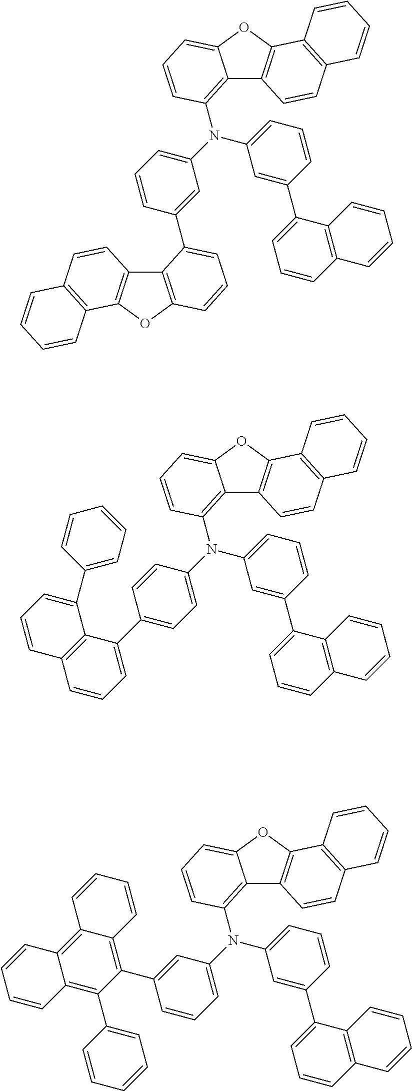 [SMILES: c1cc(-c2cccc3ccccc23)cc(N(c2cccc(-c3cccc4oc5c6ccccc6ccc5c34)c2)c2cccc3oc4c5ccccc5ccc4c23)c1 ; c1ccc(-c2cccc3cccc(-c4ccc(N(c5cccc(-c6cccc7ccccc67)c5)c5cccc6oc7c8ccccc8ccc7c56)cc4)c23)cc1 ; c1ccc(-c2c(-c3cccc(N(c4cccc(-c5cccc6ccccc56)c4)c4cccc5oc6c7ccccc7ccc6c45)c3)c3ccccc3c3ccccc23)cc1]

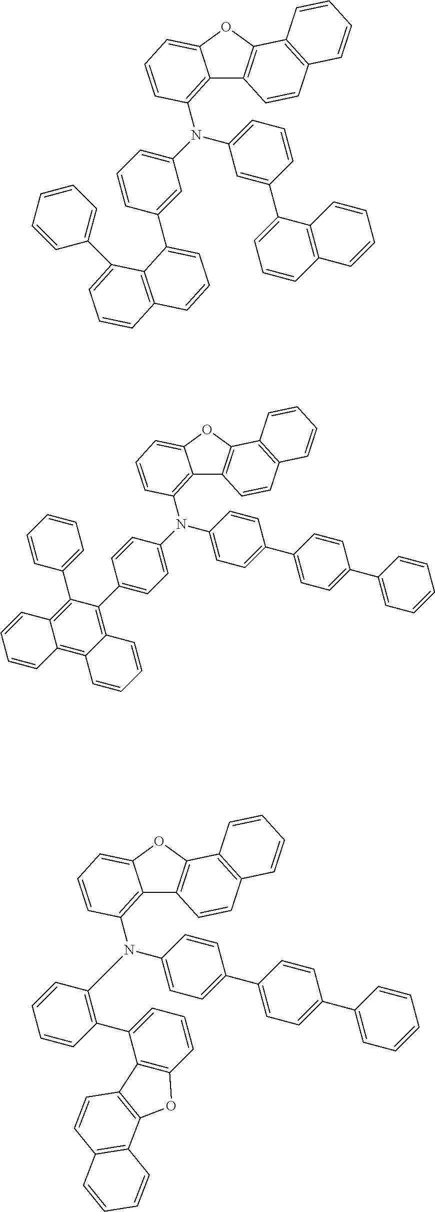 [SMILES: c1ccc(-c2cccc3cccc(-c4cccc(N(c5cccc(-c6cccc7ccccc67)c5)c5cccc6oc7c8ccccc8ccc7c56)c4)c23)cc1 ; c1ccc(-c2ccc(-c3ccc(N(c4ccc(-c5c(-c6ccccc6)c6ccccc6c6ccccc56)cc4)c4cccc5oc6c7ccccc7ccc6c45)cc3)cc2)cc1 ; c1ccc(-c2ccc(-c3ccc(N(c4ccccc4-c4cccc5oc6c7ccccc7ccc6c45)c4cccc5oc6c7ccccc7ccc6c45)cc3)cc2)cc1]

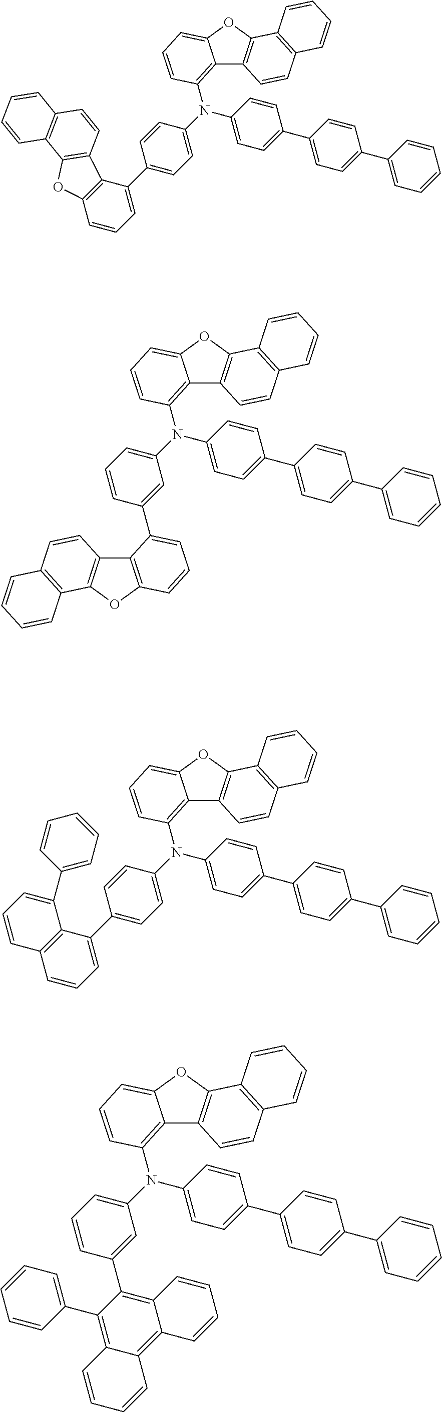 [SMILES: c1ccc(-c2ccc(-c3ccc(N(c4ccc(-c5cccc6oc7c8ccccc8ccc7c56)cc4)c4cccc5oc6c7ccccc7ccc6c45)cc3)cc2)cc1 ; c1ccc(-c2ccc(-c3ccc(N(c4cccc(-c5cccc6oc7c8ccccc8ccc7c56)c4)c4cccc5oc6c7ccccc7ccc6c45)cc3)cc2)cc1 ; c1ccc(-c2ccc(-c3ccc(N(c4ccc(-c5cccc6cccc(-c7ccccc7)c56)cc4)c4cccc5oc6c7ccccc7ccc6c45)cc3)cc2)cc1 ; c1ccc(-c2ccc(-c3ccc(N(c4cccc(-c5c(-c6ccccc6)c6ccccc6c6ccccc56)c4)c4cccc5oc6c7ccccc7ccc6c45)cc3)cc2)cc1]

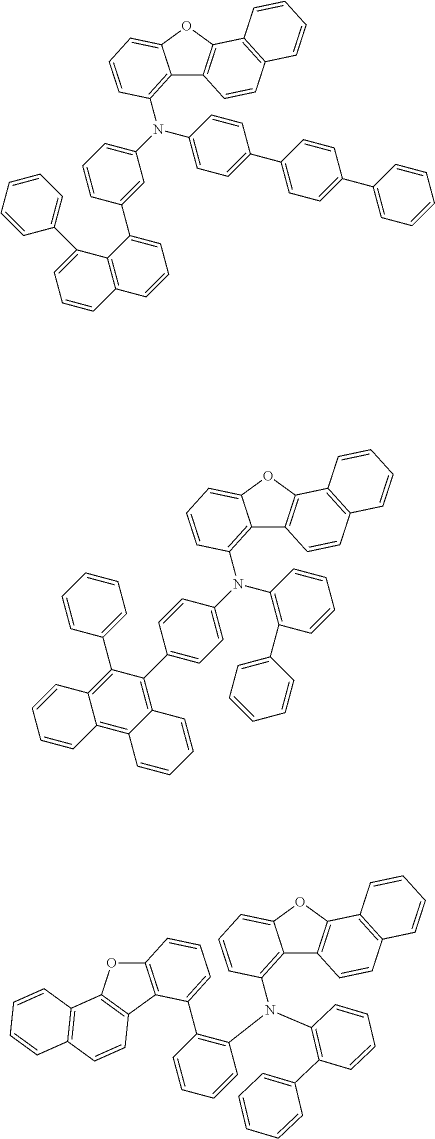 [SMILES: c1ccc(-c2ccc(-c3ccc(N(c4cccc(-c5cccc6cccc(-c7ccccc7)c56)c4)c4cccc5oc6c7ccccc7ccc6c45)cc3)cc2)cc1 ; c1ccc(-c2ccccc2N(c2ccc(-c3c(-c4ccccc4)c4ccccc4c4ccccc34)cc2)c2cccc3oc4c5ccccc5ccc4c23)cc1 ; c1ccc(-c2ccccc2N(c2ccccc2-c2cccc3oc4c5ccccc5ccc4c23)c2cccc3oc4c5ccccc5ccc4c23)cc1]

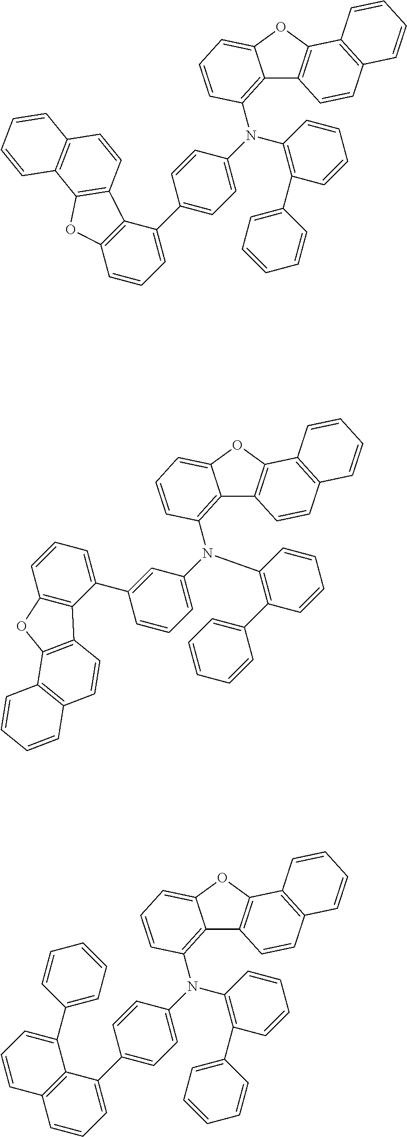 [SMILES: c1ccc(-c2ccccc2N(c2ccc(-c3cccc4oc5c6ccccc6ccc5c34)cc2)c2cccc3oc4c5ccccc5ccc4c23)cc1 ; c1ccc(-c2ccccc2N(c2cccc(-c3cccc4oc5c6ccccc6ccc5c34)c2)c2cccc3oc4c5ccccc5ccc4c23)cc1 ; c1ccc(-c2ccccc2N(c2ccc(-c3cccc4cccc(-c5ccccc5)c34)cc2)c2cccc3oc4c5ccccc5ccc4c23)cc1]

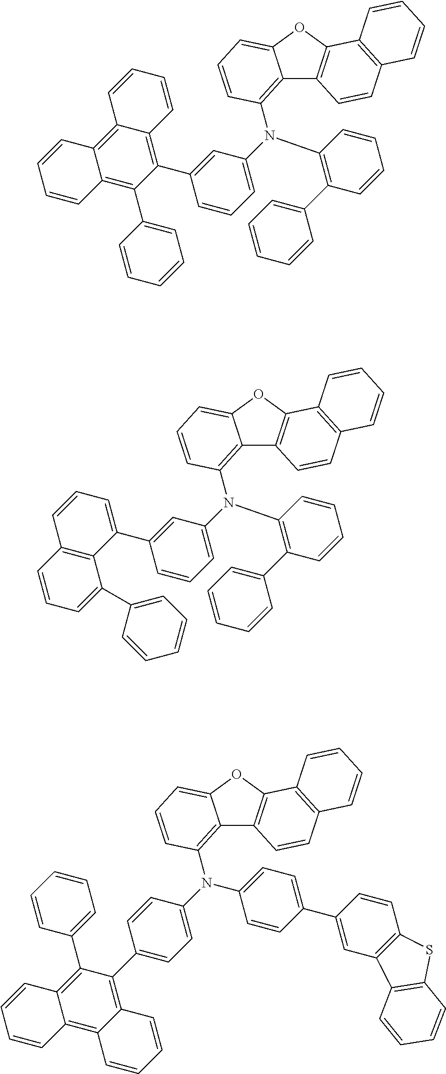 [SMILES: c1ccc(-c2ccccc2N(c2cccc(-c3c(-c4ccccc4)c4ccccc4c4ccccc34)c2)c2cccc3oc4c5ccccc5ccc4c23)cc1 ; c1ccc(-c2ccccc2N(c2cccc(-c3cccc4cccc(-c5ccccc5)c34)c2)c2cccc3oc4c5ccccc5ccc4c23)cc1 ; c1ccc(-c2c(-c3ccc(N(c4ccc(-c5ccc6sc7ccccc7c6c5)cc4)c4cccc5oc6c7ccccc7ccc6c45)cc3)c3ccccc3c3ccccc23)cc1]

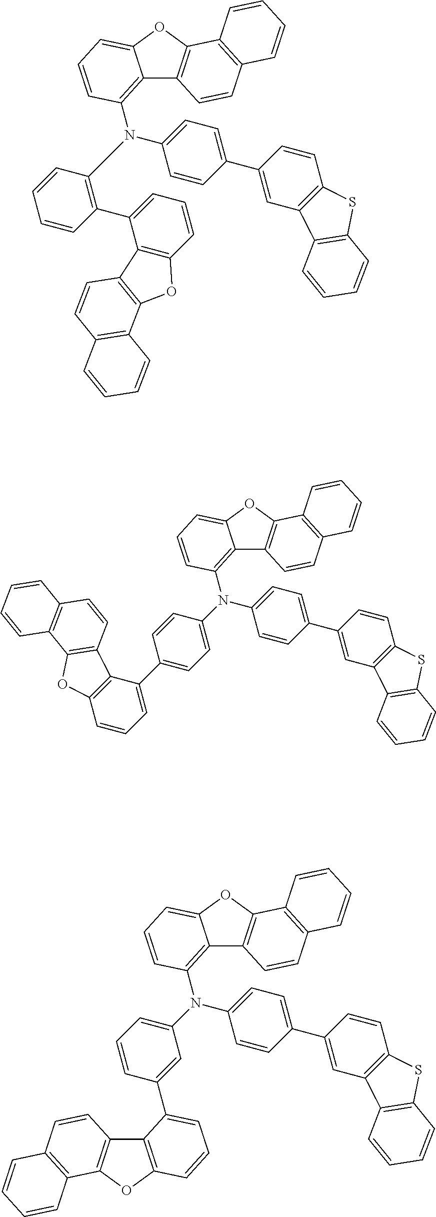 [SMILES: c1ccc(N(c2ccc(-c3ccc4sc5ccccc5c4c3)cc2)c2cccc3oc4c5ccccc5ccc4c23)c(-c2cccc3oc4c5ccccc5ccc4c23)c1 ; c1ccc2c(c1)ccc1c2oc2cccc(-c3ccc(N(c4ccc(-c5ccc6sc7ccccc7c6c5)cc4)c4cccc5oc6c7ccccc7ccc6c45)cc3)c21 ; c1cc(-c2cccc3oc4c5ccccc5ccc4c23)cc(N(c2ccc(-c3ccc4sc5ccccc5c4c3)cc2)c2cccc3oc4c5ccccc5ccc4c23)c1]

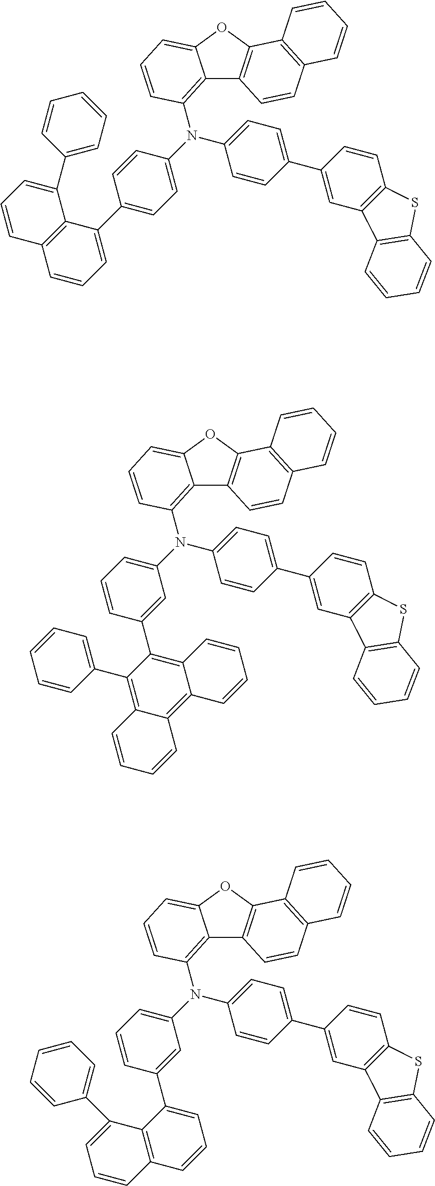 [SMILES: c1ccc(-c2cccc3cccc(-c4ccc(N(c5ccc(-c6ccc7sc8ccccc8c7c6)cc5)c5cccc6oc7c8ccccc8ccc7c56)cc4)c23)cc1 ; c1ccc(-c2c(-c3cccc(N(c4ccc(-c5ccc6sc7ccccc7c6c5)cc4)c4cccc5oc6c7ccccc7ccc6c45)c3)c3ccccc3c3ccccc23)cc1 ; c1ccc(-c2cccc3cccc(-c4cccc(N(c5ccc(-c6ccc7sc8ccccc8c7c6)cc5)c5cccc6oc7c8ccccc8ccc7c56)c4)c23)cc1]

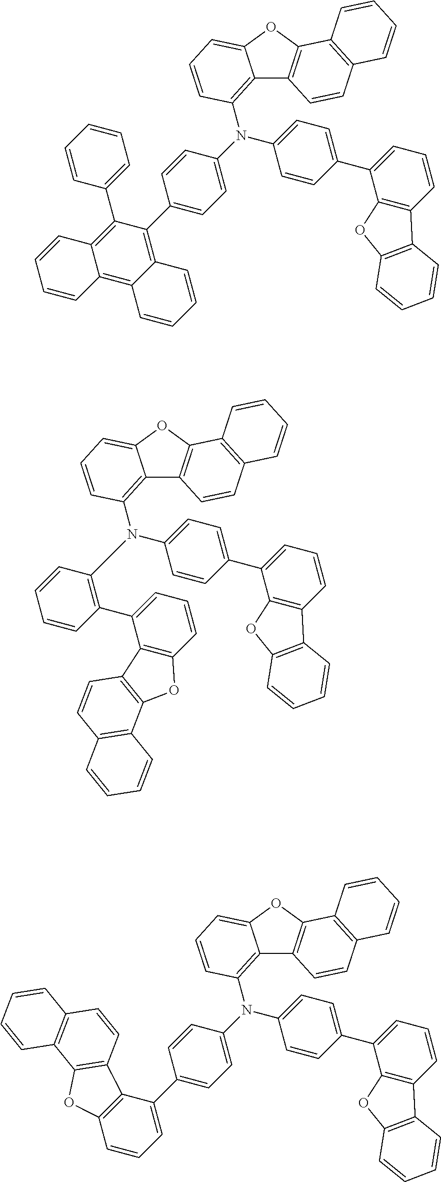 [SMILES: c1ccc(-c2c(-c3ccc(N(c4ccc(-c5cccc6c5oc5ccccc56)cc4)c4cccc5oc6c7ccccc7ccc6c45)cc3)c3ccccc3c3ccccc23)cc1 ; c1ccc(N(c2ccc(-c3cccc4c3oc3ccccc34)cc2)c2cccc3oc4c5ccccc5ccc4c23)c(-c2cccc3oc4c5ccccc5ccc4c23)c1 ; c1ccc2c(c1)ccc1c2oc2cccc(-c3ccc(N(c4ccc(-c5cccc6c5oc5ccccc56)cc4)c4cccc5oc6c7ccccc7ccc6c45)cc3)c21]

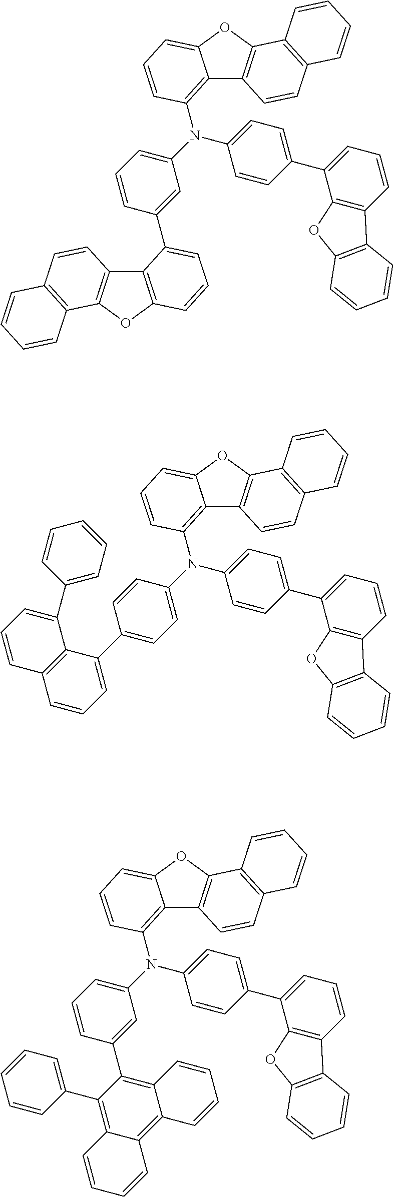 [SMILES: c1cc(-c2cccc3oc4c5ccccc5ccc4c23)cc(N(c2ccc(-c3cccc4c3oc3ccccc34)cc2)c2cccc3oc4c5ccccc5ccc4c23)c1 ; c1ccc(-c2cccc3cccc(-c4ccc(N(c5ccc(-c6cccc7c6oc6ccccc67)cc5)c5cccc6oc7c8ccccc8ccc7c56)cc4)c23)cc1 ; c1ccc(-c2c(-c3cccc(N(c4ccc(-c5cccc6c5oc5ccccc56)cc4)c4cccc5oc6c7ccccc7ccc6c45)c3)c3ccccc3c3ccccc23)cc1]

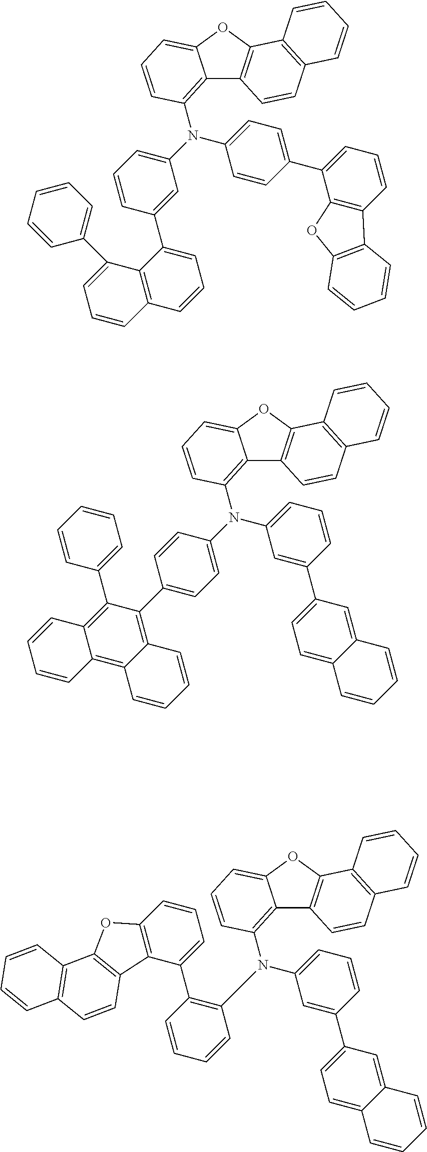 [SMILES: c1ccc(-c2cccc3cccc(-c4cccc(N(c5ccc(-c6cccc7c6oc6ccccc67)cc5)c5cccc6oc7c8ccccc8ccc7c56)c4)c23)cc1 ; c1ccc(-c2c(-c3ccc(N(c4cccc(-c5ccc6ccccc6c5)c4)c4cccc5oc6c7ccccc7ccc6c45)cc3)c3ccccc3c3ccccc23)cc1 ; c1cc(-c2ccc3ccccc3c2)cc(N(c2ccccc2-c2cccc3oc4c5ccccc5ccc4c23)c2cccc3oc4c5ccccc5ccc4c23)c1]

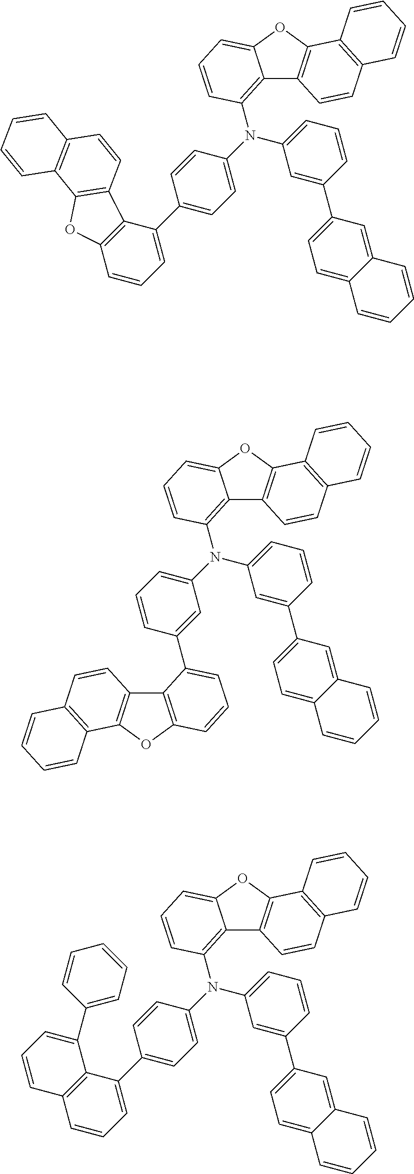 [SMILES: c1cc(-c2ccc3ccccc3c2)cc(N(c2ccc(-c3cccc4oc5c6ccccc6ccc5c34)cc2)c2cccc3oc4c5ccccc5ccc4c23)c1 ; c1cc(-c2ccc3ccccc3c2)cc(N(c2cccc(-c3cccc4oc5c6ccccc6ccc5c34)c2)c2cccc3oc4c5ccccc5ccc4c23)c1 ; c1ccc(-c2cccc3cccc(-c4ccc(N(c5cccc(-c6ccc7ccccc7c6)c5)c5cccc6oc7c8ccccc8ccc7c56)cc4)c23)cc1]

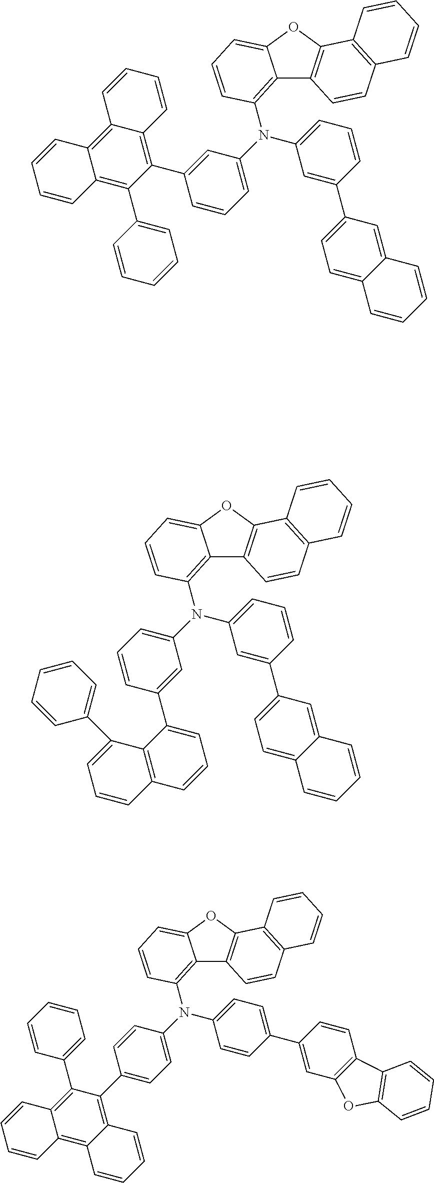 [SMILES: c1ccc(-c2c(-c3cccc(N(c4cccc(-c5ccc6ccccc6c5)c4)c4cccc5oc6c7ccccc7ccc6c45)c3)c3ccccc3c3ccccc23)cc1 ; c1ccc(-c2cccc3cccc(-c4cccc(N(c5cccc(-c6ccc7ccccc7c6)c5)c5cccc6oc7c8ccccc8ccc7c56)c4)c23)cc1 ; c1ccc(-c2c(-c3ccc(N(c4ccc(-c5ccc6c(c5)oc5ccccc56)cc4)c4cccc5oc6c7ccccc7ccc6c45)cc3)c3ccccc3c3ccccc23)cc1]

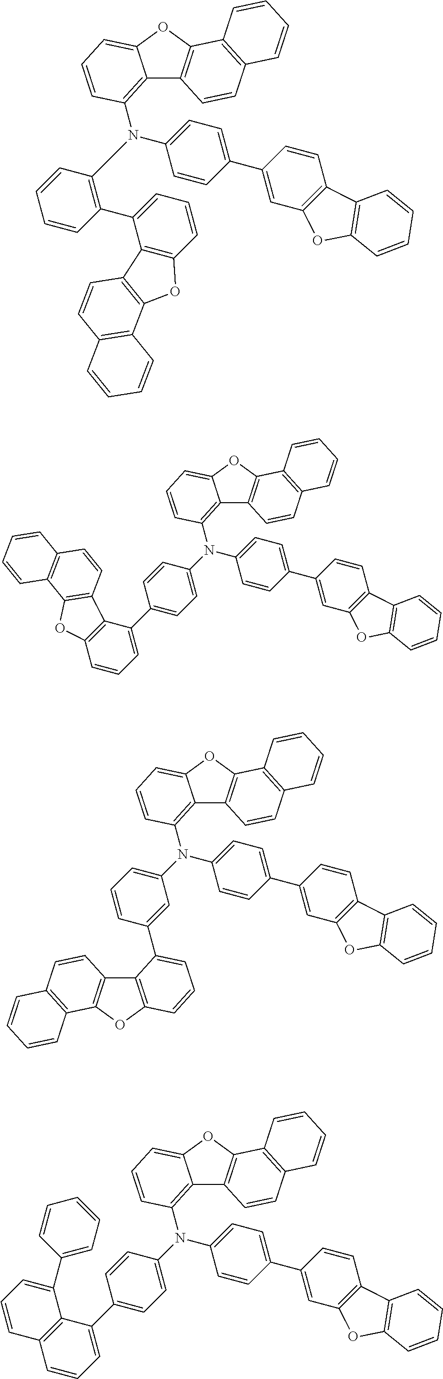 [SMILES: c1ccc(N(c2ccc(-c3ccc4c(c3)oc3ccccc34)cc2)c2cccc3oc4c5ccccc5ccc4c23)c(-c2cccc3oc4c5ccccc5ccc4c23)c1 ; c1ccc2c(c1)ccc1c2oc2cccc(-c3ccc(N(c4ccc(-c5ccc6c(c5)oc5ccccc56)cc4)c4cccc5oc6c7ccccc7ccc6c45)cc3)c21 ; c1cc(-c2cccc3oc4c5ccccc5ccc4c23)cc(N(c2ccc(-c3ccc4c(c3)oc3ccccc34)cc2)c2cccc3oc4c5ccccc5ccc4c23)c1 ; c1ccc(-c2cccc3cccc(-c4ccc(N(c5ccc(-c6ccc7c(c6)oc6ccccc67)cc5)c5cccc6oc7c8ccccc8ccc7c56)cc4)c23)cc1]

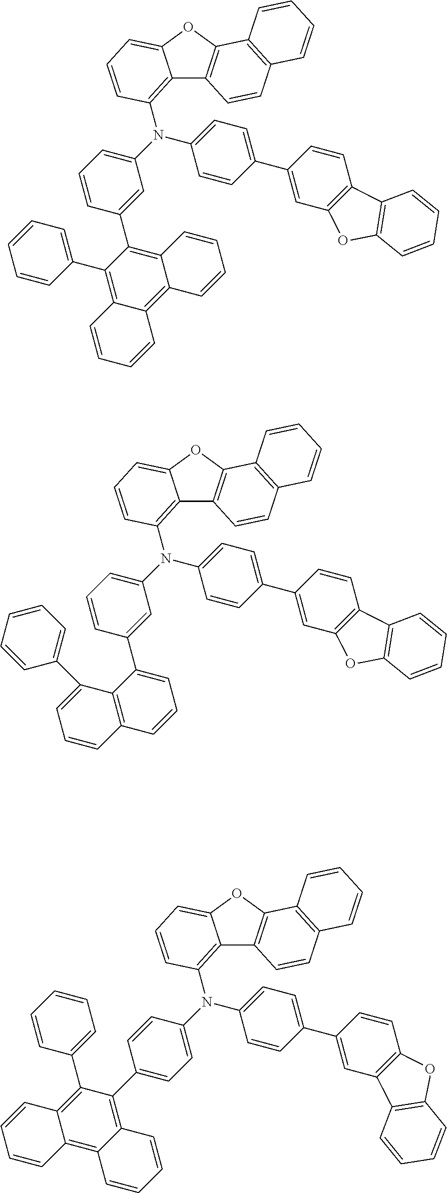 [SMILES: c1ccc(-c2c(-c3cccc(N(c4ccc(-c5ccc6c(c5)oc5ccccc56)cc4)c4cccc5oc6c7ccccc7ccc6c45)c3)c3ccccc3c3ccccc23)cc1 ; c1ccc(-c2cccc3cccc(-c4cccc(N(c5ccc(-c6ccc7c(c6)oc6ccccc67)cc5)c5cccc6oc7c8ccccc8ccc7c56)c4)c23)cc1 ; c1ccc(-c2c(-c3ccc(N(c4ccc(-c5ccc6oc7ccccc7c6c5)cc4)c4cccc5oc6c7ccccc7ccc6c45)cc3)c3ccccc3c3ccccc23)cc1]

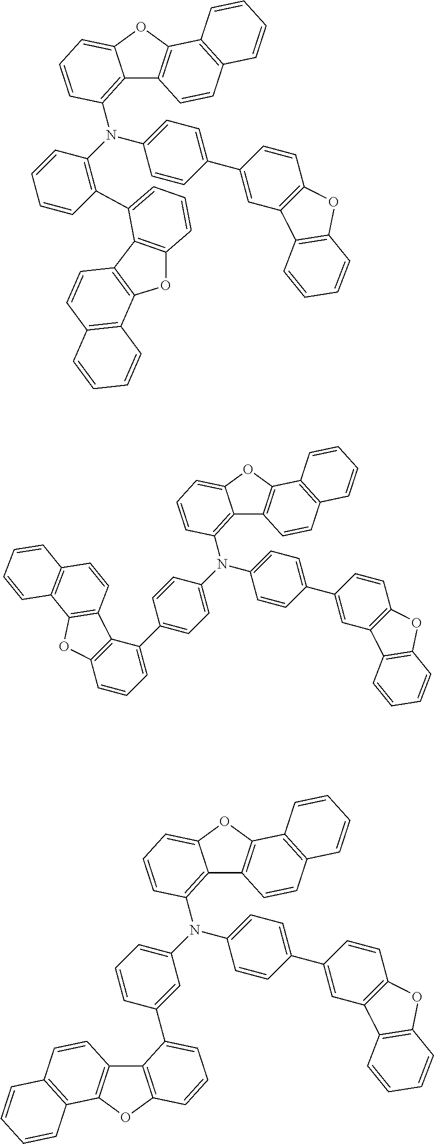 [SMILES: c1ccc(N(c2ccc(-c3ccc4oc5ccccc5c4c3)cc2)c2cccc3oc4c5ccccc5ccc4c23)c(-c2cccc3oc4c5ccccc5ccc4c23)c1 ; c1ccc2c(c1)ccc1c2oc2cccc(-c3ccc(N(c4ccc(-c5ccc6oc7ccccc7c6c5)cc4)c4cccc5oc6c7ccccc7ccc6c45)cc3)c21 ; c1cc(-c2cccc3oc4c5ccccc5ccc4c23)cc(N(c2ccc(-c3ccc4oc5ccccc5c4c3)cc2)c2cccc3oc4c5ccccc5ccc4c23)c1]

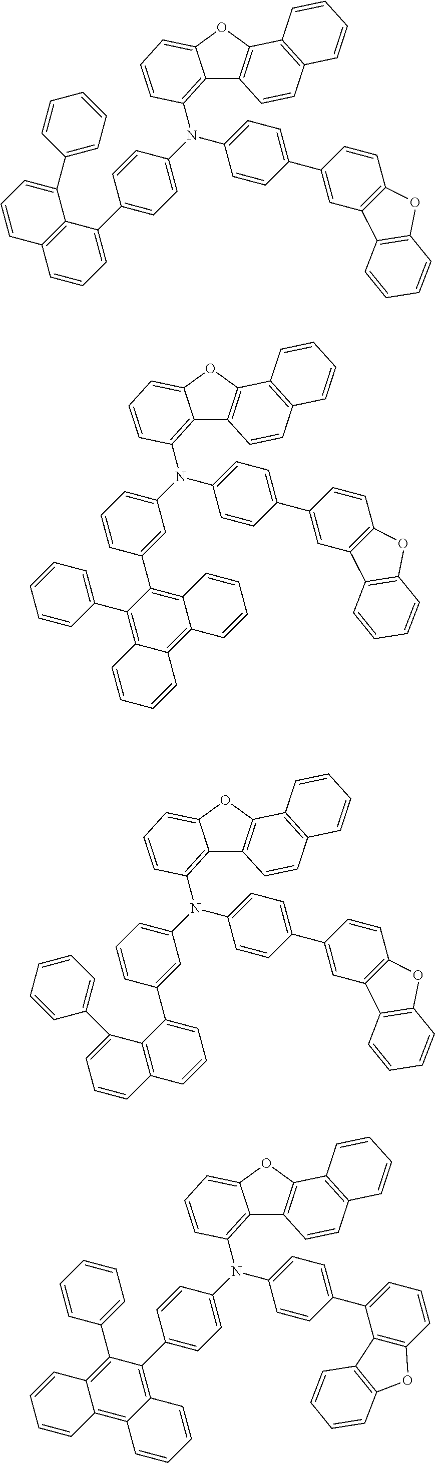 [SMILES: c1ccc(-c2cccc3cccc(-c4ccc(N(c5ccc(-c6ccc7oc8ccccc8c7c6)cc5)c5cccc6oc7c8ccccc8ccc7c56)cc4)c23)cc1 ; c1ccc(-c2c(-c3cccc(N(c4ccc(-c5ccc6oc7ccccc7c6c5)cc4)c4cccc5oc6c7ccccc7ccc6c45)c3)c3ccccc3c3ccccc23)cc1 ; c1ccc(-c2cccc3cccc(-c4cccc(N(c5ccc(-c6ccc7oc8ccccc8c7c6)cc5)c5cccc6oc7c8ccccc8ccc7c56)c4)c23)cc1 ; c1ccc(-c2c(-c3ccc(N(c4ccc(-c5cccc6oc7ccccc7c56)cc4)c4cccc5oc6c7ccccc7ccc6c45)cc3)c3ccccc3c3ccccc23)cc1]

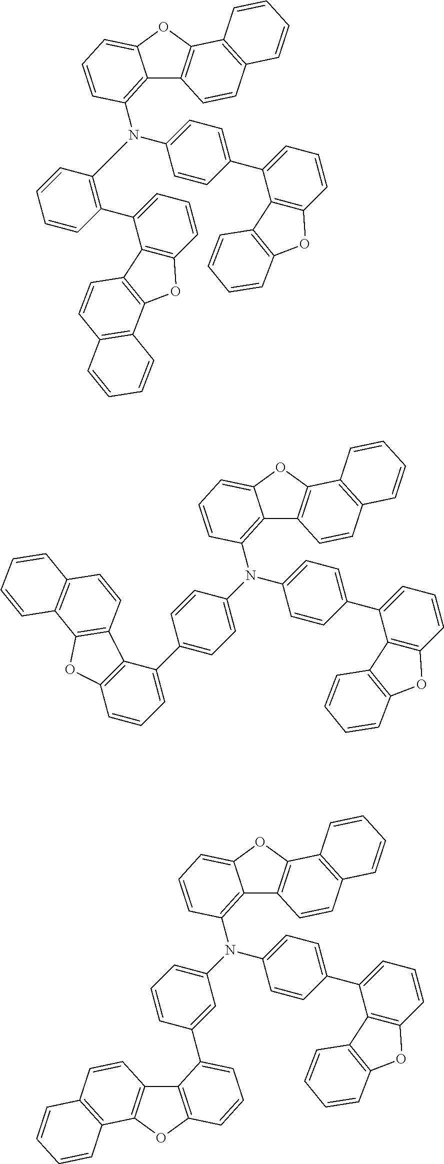 [SMILES: c1ccc(N(c2ccc(-c3cccc4oc5ccccc5c34)cc2)c2cccc3oc4c5ccccc5ccc4c23)c(-c2cccc3oc4c5ccccc5ccc4c23)c1 ; c1ccc2c(c1)ccc1c2oc2cccc(-c3ccc(N(c4ccc(-c5cccc6oc7ccccc7c56)cc4)c4cccc5oc6c7ccccc7ccc6c45)cc3)c21 ; c1cc(-c2cccc3oc4c5ccccc5ccc4c23)cc(N(c2ccc(-c3cccc4oc5ccccc5c34)cc2)c2cccc3oc4c5ccccc5ccc4c23)c1]

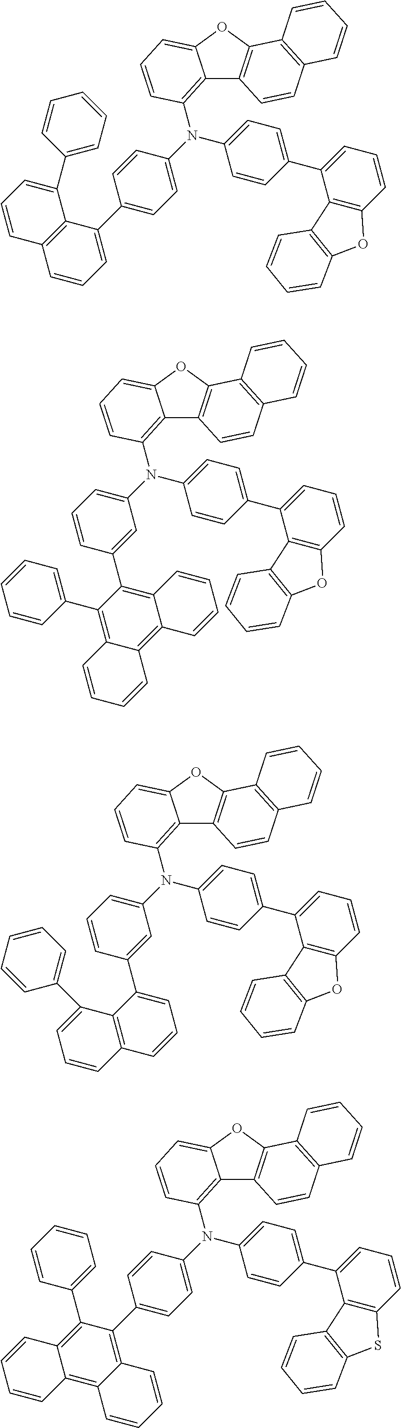 [SMILES: c1ccc(-c2cccc3cccc(-c4ccc(N(c5ccc(-c6cccc7oc8ccccc8c67)cc5)c5cccc6oc7c8ccccc8ccc7c56)cc4)c23)cc1 ; c1ccc(-c2c(-c3cccc(N(c4ccc(-c5cccc6oc7ccccc7c56)cc4)c4cccc5oc6c7ccccc7ccc6c45)c3)c3ccccc3c3ccccc23)cc1 ; c1ccc(-c2cccc3cccc(-c4cccc(N(c5ccc(-c6cccc7oc8ccccc8c67)cc5)c5cccc6oc7c8ccccc8ccc7c56)c4)c23)cc1 ; c1ccc(-c2c(-c3ccc(N(c4ccc(-c5cccc6sc7ccccc7c56)cc4)c4cccc5oc6c7ccccc7ccc6c45)cc3)c3ccccc3c3ccccc23)cc1]

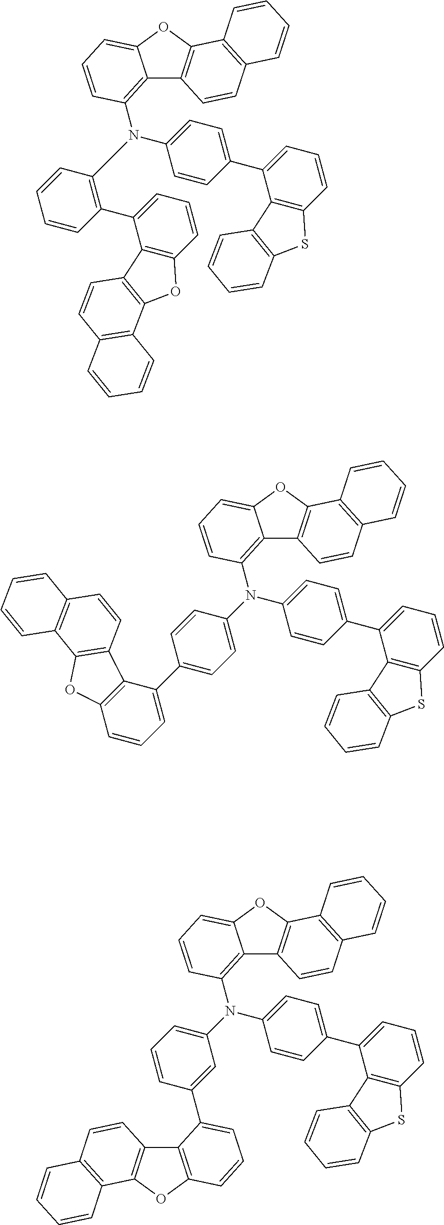 [SMILES: c1ccc(N(c2ccc(-c3cccc4sc5ccccc5c34)cc2)c2cccc3oc4c5ccccc5ccc4c23)c(-c2cccc3oc4c5ccccc5ccc4c23)c1 ; c1ccc2c(c1)ccc1c2oc2cccc(-c3ccc(N(c4ccc(-c5cccc6sc7ccccc7c56)cc4)c4cccc5oc6c7ccccc7ccc6c45)cc3)c21 ; c1cc(-c2cccc3oc4c5ccccc5ccc4c23)cc(N(c2ccc(-c3cccc4sc5ccccc5c34)cc2)c2cccc3oc4c5ccccc5ccc4c23)c1]

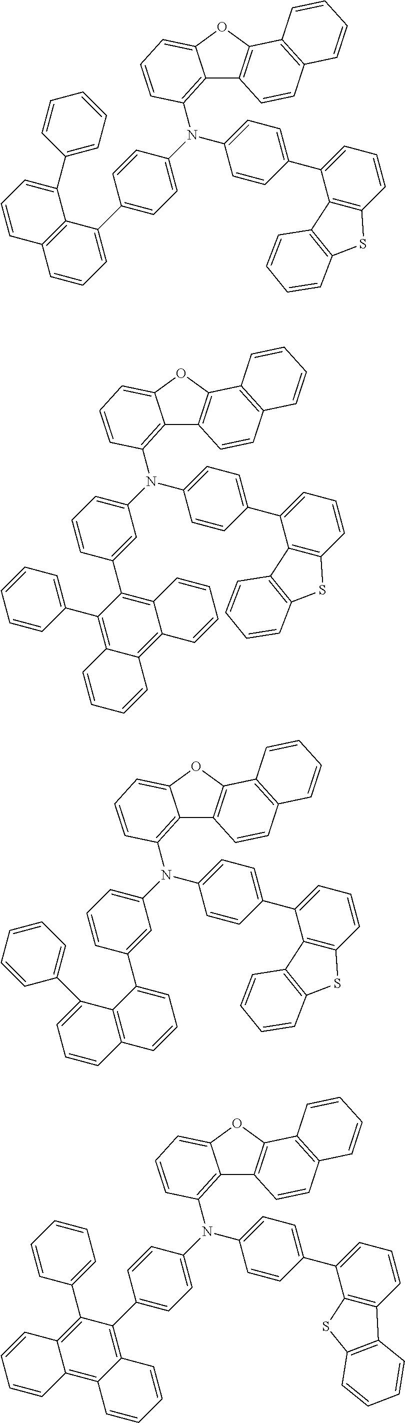 [SMILES: c1ccc(-c2cccc3cccc(-c4ccc(N(c5ccc(-c6cccc7sc8ccccc8c67)cc5)c5cccc6oc7c8ccccc8ccc7c56)cc4)c23)cc1 ; c1ccc(-c2c(-c3cccc(N(c4ccc(-c5cccc6sc7ccccc7c56)cc4)c4cccc5oc6c7ccccc7ccc6c45)c3)c3ccccc3c3ccccc23)cc1 ; c1ccc(-c2cccc3cccc(-c4cccc(N(c5ccc(-c6cccc7sc8ccccc8c67)cc5)c5cccc6oc7c8ccccc8ccc7c56)c4)c23)cc1 ; c1ccc(-c2c(-c3ccc(N(c4ccc(-c5cccc6c5sc5ccccc56)cc4)c4cccc5oc6c7ccccc7ccc6c45)cc3)c3ccccc3c3ccccc23)cc1]

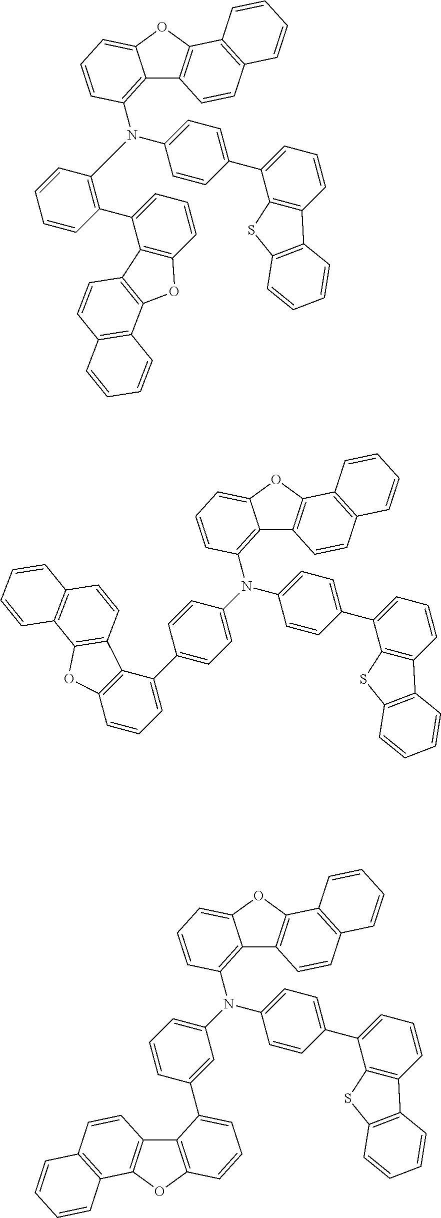 [SMILES: c1ccc(N(c2ccc(-c3cccc4c3sc3ccccc34)cc2)c2cccc3oc4c5ccccc5ccc4c23)c(-c2cccc3oc4c5ccccc5ccc4c23)c1 ; c1ccc2c(c1)ccc1c2oc2cccc(-c3ccc(N(c4ccc(-c5cccc6c5sc5ccccc56)cc4)c4cccc5oc6c7ccccc7ccc6c45)cc3)c21 ; c1cc(-c2cccc3oc4c5ccccc5ccc4c23)cc(N(c2ccc(-c3cccc4c3sc3ccccc34)cc2)c2cccc3oc4c5ccccc5ccc4c23)c1]

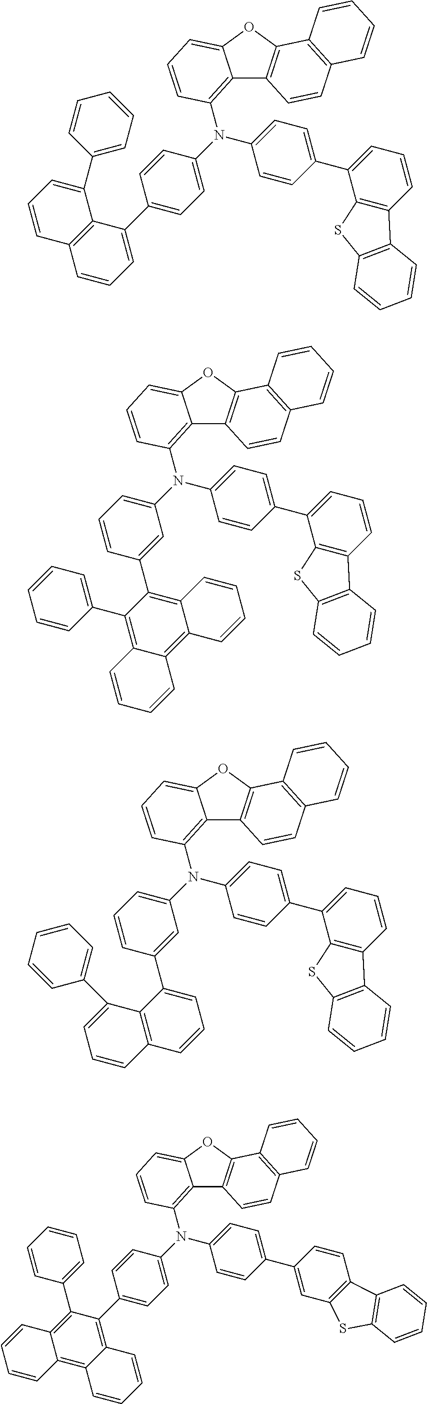 [SMILES: c1ccc(-c2cccc3cccc(-c4ccc(N(c5ccc(-c6cccc7c6sc6ccccc67)cc5)c5cccc6oc7c8ccccc8ccc7c56)cc4)c23)cc1 ; c1ccc(-c2c(-c3cccc(N(c4ccc(-c5cccc6c5sc5ccccc56)cc4)c4cccc5oc6c7ccccc7ccc6c45)c3)c3ccccc3c3ccccc23)cc1 ; c1ccc(-c2cccc3cccc(-c4cccc(N(c5ccc(-c6cccc7c6sc6ccccc67)cc5)c5cccc6oc7c8ccccc8ccc7c56)c4)c23)cc1 ; c1ccc(-c2c(-c3ccc(N(c4ccc(-c5ccc6c(c5)sc5ccccc56)cc4)c4cccc5oc6c7ccccc7ccc6c45)cc3)c3ccccc3c3ccccc23)cc1]

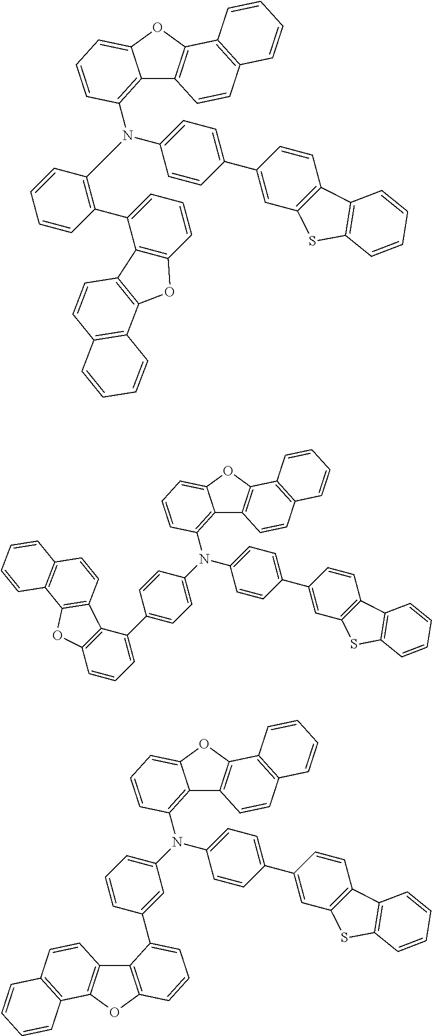 [SMILES: c1ccc(N(c2ccc(-c3ccc4c(c3)sc3ccccc34)cc2)c2cccc3oc4c5ccccc5ccc4c23)c(-c2cccc3oc4c5ccccc5ccc4c23)c1 ; c1ccc2c(c1)ccc1c2oc2cccc(-c3ccc(N(c4ccc(-c5ccc6c(c5)sc5ccccc56)cc4)c4cccc5oc6c7ccccc7ccc6c45)cc3)c21 ; c1cc(-c2cccc3oc4c5ccccc5ccc4c23)cc(N(c2ccc(-c3ccc4c(c3)sc3ccccc34)cc2)c2cccc3oc4c5ccccc5ccc4c23)c1]

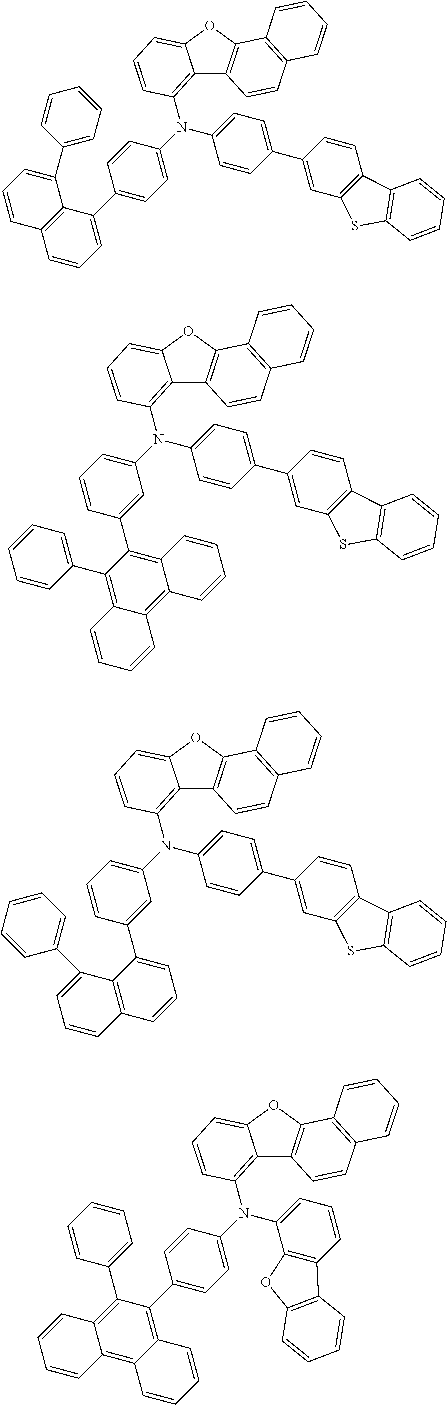 [SMILES: c1ccc(-c2cccc3cccc(-c4ccc(N(c5ccc(-c6ccc7c(c6)sc6ccccc67)cc5)c5cccc6oc7c8ccccc8ccc7c56)cc4)c23)cc1 ; c1ccc(-c2c(-c3cccc(N(c4ccc(-c5ccc6c(c5)sc5ccccc56)cc4)c4cccc5oc6c7ccccc7ccc6c45)c3)c3ccccc3c3ccccc23)cc1 ; c1ccc(-c2cccc3cccc(-c4cccc(N(c5ccc(-c6ccc7c(c6)sc6ccccc67)cc5)c5cccc6oc7c8ccccc8ccc7c56)c4)c23)cc1 ; c1ccc(-c2c(-c3ccc(N(c4cccc5c4oc4ccccc45)c4cccc5oc6c7ccccc7ccc6c45)cc3)c3ccccc3c3ccccc23)cc1]

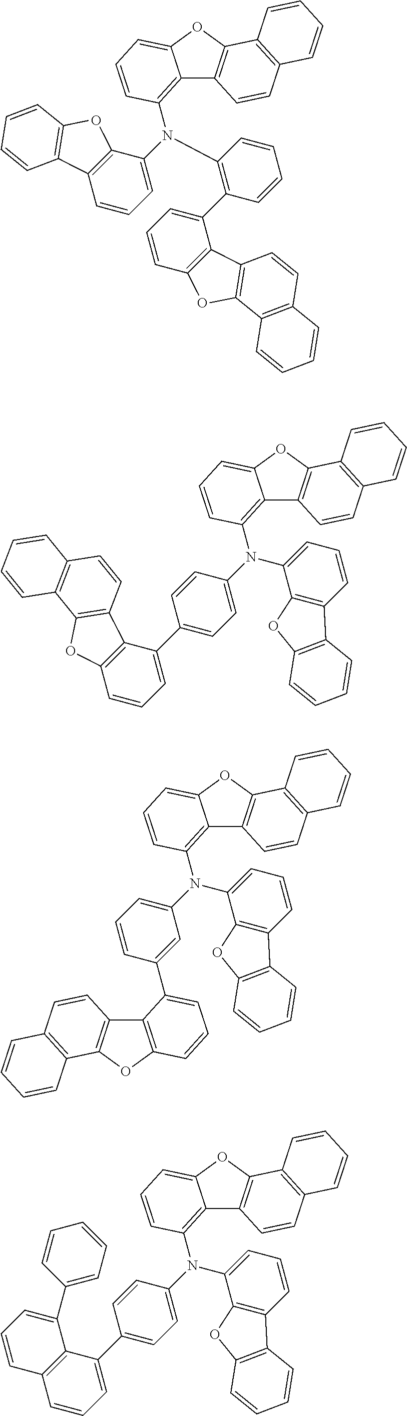

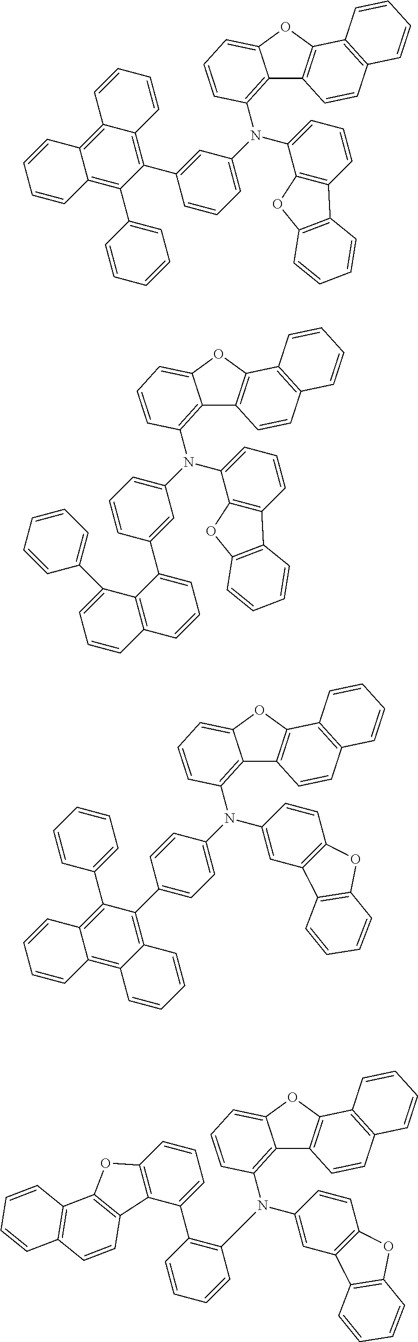

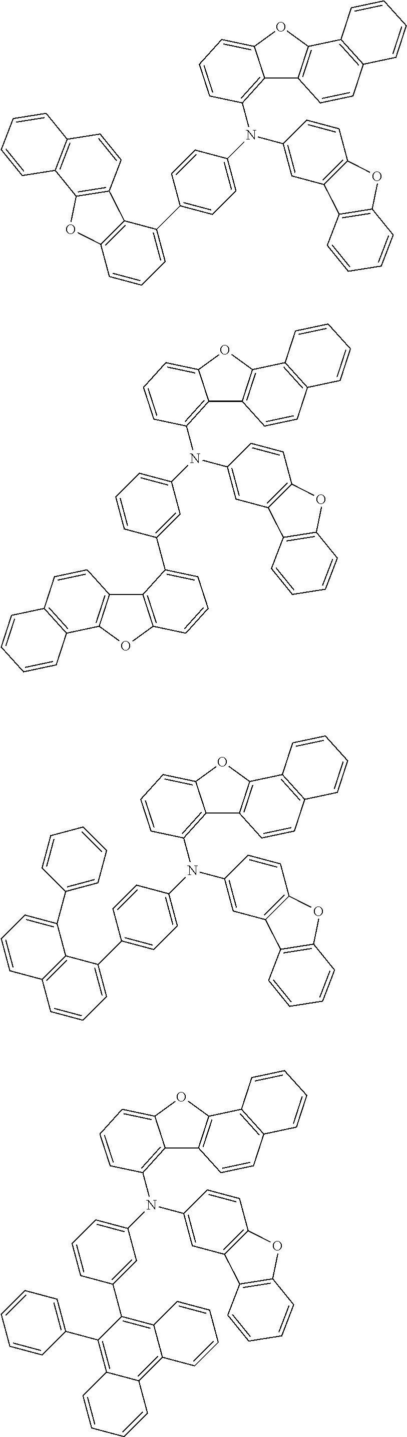 [SMILES: c1ccc2c(c1)ccc1c2oc2cccc(-c3ccc(N(c4ccc5oc6ccccc6c5c4)c4cccc5oc6c7ccccc7ccc6c45)cc3)c21 ; c1cc(-c2cccc3oc4c5ccccc5ccc4c23)cc(N(c2ccc3oc4ccccc4c3c2)c2cccc3oc4c5ccccc5ccc4c23)c1 ; c1ccc(-c2cccc3cccc(-c4ccc(N(c5ccc6oc7ccccc7c6c5)c5cccc6oc7c8ccccc8ccc7c56)cc4)c23)cc1 ; c1ccc(-c2c(-c3cccc(N(c4ccc5oc6ccccc6c5c4)c4cccc5oc6c7ccccc7ccc6c45)c3)c3ccccc3c3ccccc23)cc1]

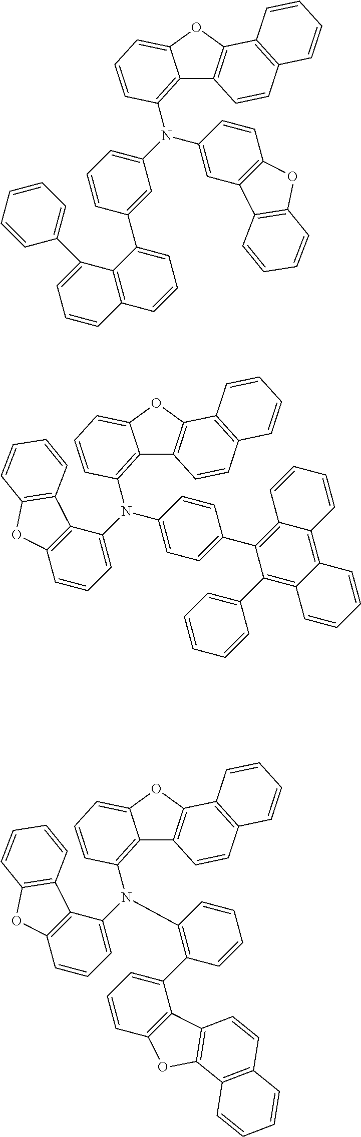 [SMILES: c1ccc(-c2cccc3cccc(-c4cccc(N(c5ccc6oc7ccccc7c6c5)c5cccc6oc7c8ccccc8ccc7c56)c4)c23)cc1 ; c1ccc(-c2c(-c3ccc(N(c4cccc5oc6ccccc6c45)c4cccc5oc6c7ccccc7ccc6c45)cc3)c3ccccc3c3ccccc23)cc1 ; c1ccc(N(c2cccc3oc4ccccc4c23)c2cccc3oc4c5ccccc5ccc4c23)c(-c2cccc3oc4c5ccccc5ccc4c23)c1]

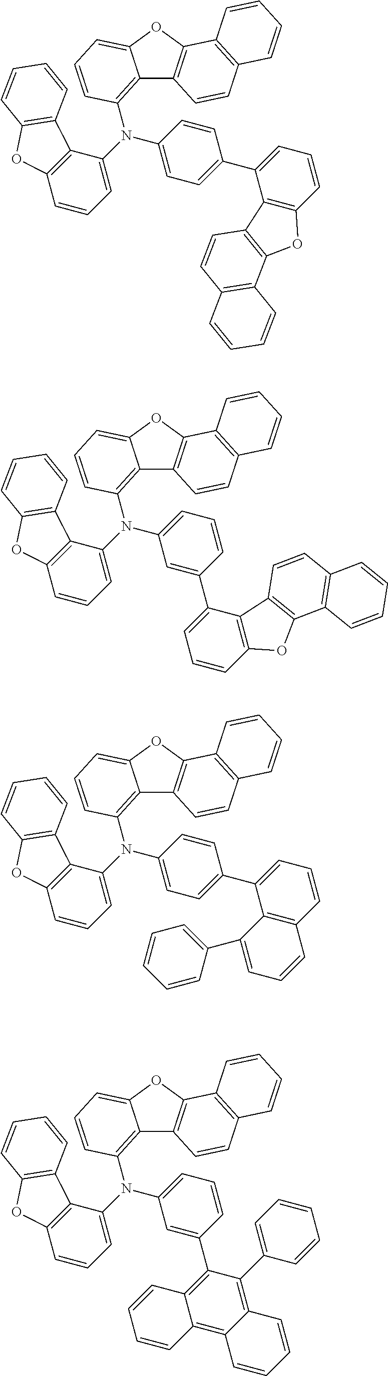 [SMILES: c1ccc2c(c1)ccc1c2oc2cccc(-c3ccc(N(c4cccc5oc6ccccc6c45)c4cccc5oc6c7ccccc7ccc6c45)cc3)c21 ; c1cc(-c2cccc3oc4c5ccccc5ccc4c23)cc(N(c2cccc3oc4ccccc4c23)c2cccc3oc4c5ccccc5ccc4c23)c1 ; c1ccc(-c2cccc3cccc(-c4ccc(N(c5cccc6oc7ccccc7c56)c5cccc6oc7c8ccccc8ccc7c56)cc4)c23)cc1 ; c1ccc(-c2c(-c3cccc(N(c4cccc5oc6ccccc6c45)c4cccc5oc6c7ccccc7ccc6c45)c3)c3ccccc3c3ccccc23)cc1]

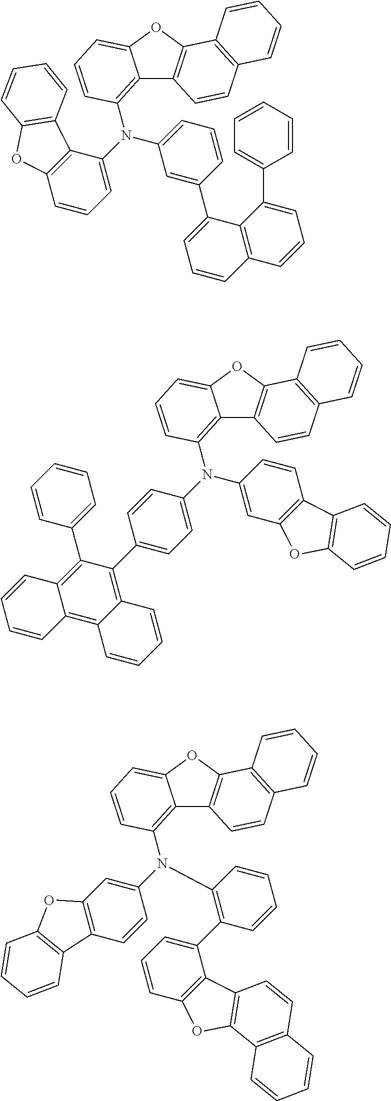 [SMILES: c1ccc(-c2cccc3cccc(-c4cccc(N(c5cccc6oc7ccccc7c56)c5cccc6oc7c8ccccc8ccc7c56)c4)c23)cc1 ; c1ccc(-c2c(-c3ccc(N(c4ccc5c(c4)oc4ccccc45)c4cccc5oc6c7ccccc7ccc6c45)cc3)c3ccccc3c3ccccc23)cc1 ; c1ccc(N(c2ccc3c(c2)oc2ccccc23)c2cccc3oc4c5ccccc5ccc4c23)c(-c2cccc3oc4c5ccccc5ccc4c23)c1]

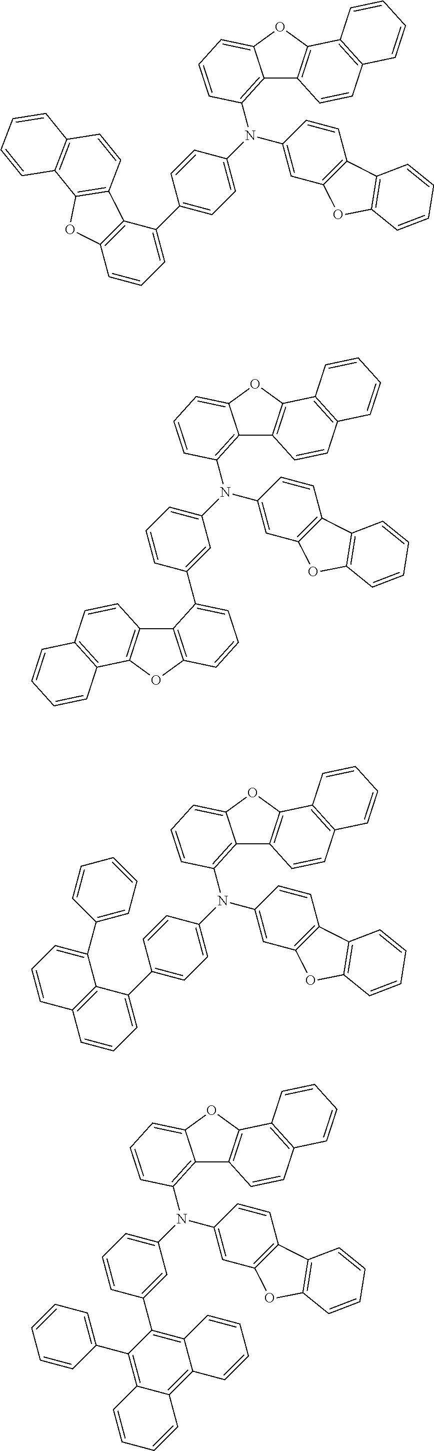 [SMILES: c1ccc2c(c1)ccc1c2oc2cccc(-c3ccc(N(c4ccc5c(c4)oc4ccccc45)c4cccc5oc6c7ccccc7ccc6c45)cc3)c21 ; c1cc(-c2cccc3oc4c5ccccc5ccc4c23)cc(N(c2ccc3c(c2)oc2ccccc23)c2cccc3oc4c5ccccc5ccc4c23)c1 ; c1ccc(-c2cccc3cccc(-c4ccc(N(c5ccc6c(c5)oc5ccccc56)c5cccc6oc7c8ccccc8ccc7c56)cc4)c23)cc1 ; c1ccc(-c2c(-c3cccc(N(c4ccc5c(c4)oc4ccccc45)c4cccc5oc6c7ccccc7ccc6c45)c3)c3ccccc3c3ccccc23)cc1]

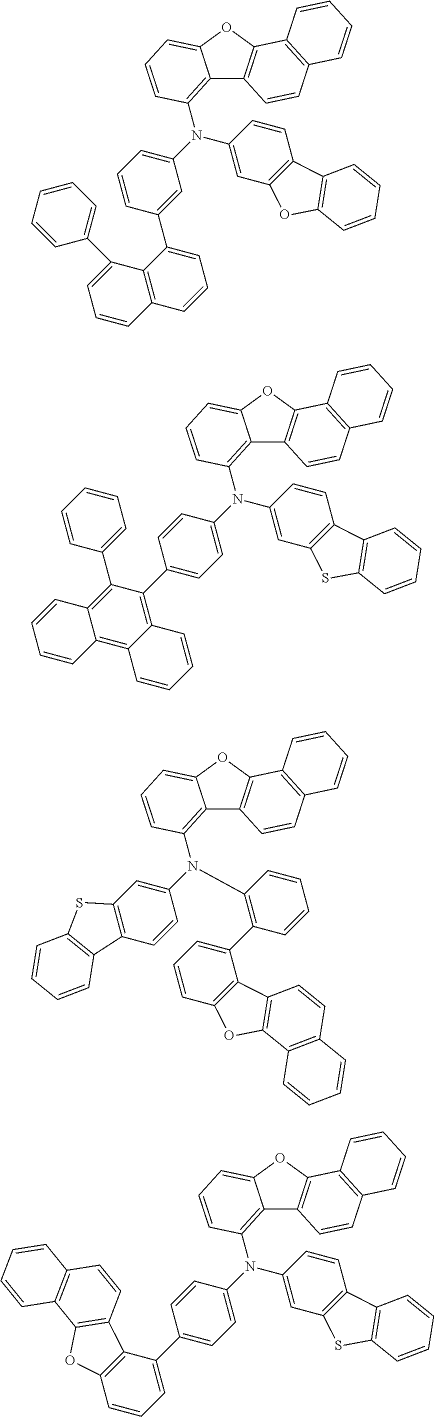 [SMILES: c1ccc(-c2cccc3cccc(-c4cccc(N(c5ccc6c(c5)oc5ccccc56)c5cccc6oc7c8ccccc8ccc7c56)c4)c23)cc1 ; c1ccc(-c2c(-c3ccc(N(c4ccc5c(c4)sc4ccccc45)c4cccc5oc6c7ccccc7ccc6c45)cc3)c3ccccc3c3ccccc23)cc1 ; c1ccc(N(c2ccc3c(c2)sc2ccccc23)c2cccc3oc4c5ccccc5ccc4c23)c(-c2cccc3oc4c5ccccc5ccc4c23)c1 ; c1ccc2c(c1)ccc1c2oc2cccc(-c3ccc(N(c4ccc5c(c4)sc4ccccc45)c4cccc5oc6c7ccccc7ccc6c45)cc3)c21]

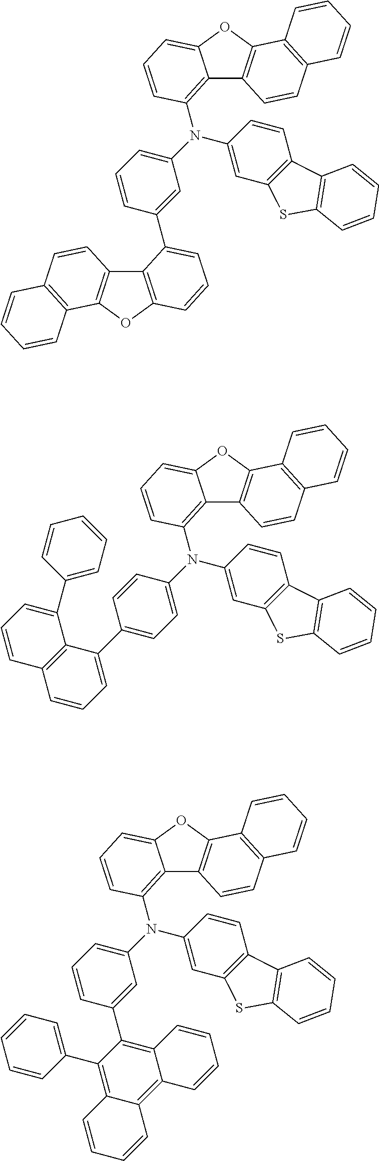 [SMILES: c1cc(-c2cccc3oc4c5ccccc5ccc4c23)cc(N(c2ccc3c(c2)sc2ccccc23)c2cccc3oc4c5ccccc5ccc4c23)c1 ; c1ccc(-c2cccc3cccc(-c4ccc(N(c5ccc6c(c5)sc5ccccc56)c5cccc6oc7c8ccccc8ccc7c56)cc4)c23)cc1 ; c1ccc(-c2c(-c3cccc(N(c4ccc5c(c4)sc4ccccc45)c4cccc5oc6c7ccccc7ccc6c45)c3)c3ccccc3c3ccccc23)cc1]

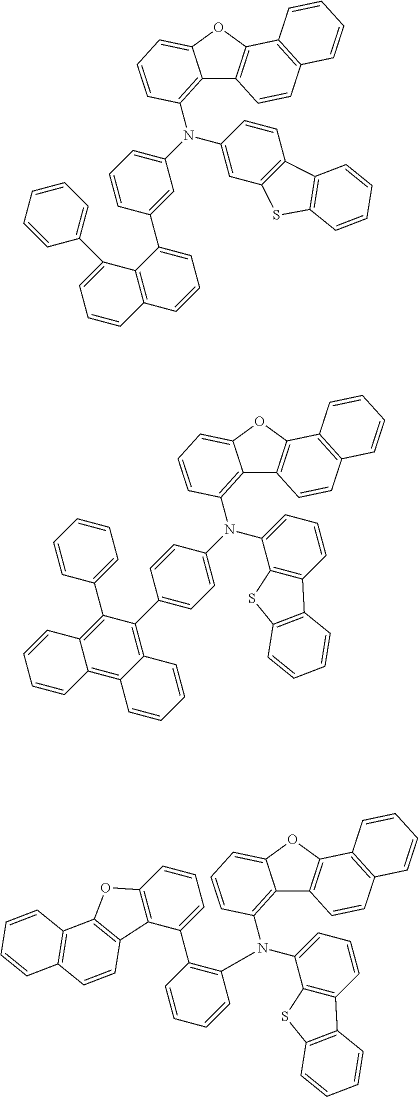 [SMILES: c1ccc(-c2cccc3cccc(-c4cccc(N(c5ccc6c(c5)sc5ccccc56)c5cccc6oc7c8ccccc8ccc7c56)c4)c23)cc1 ; c1ccc(-c2c(-c3ccc(N(c4cccc5c4sc4ccccc45)c4cccc5oc6c7ccccc7ccc6c45)cc3)c3ccccc3c3ccccc23)cc1 ; c1ccc(N(c2cccc3c2sc2ccccc23)c2cccc3oc4c5ccccc5ccc4c23)c(-c2cccc3oc4c5ccccc5ccc4c23)c1]

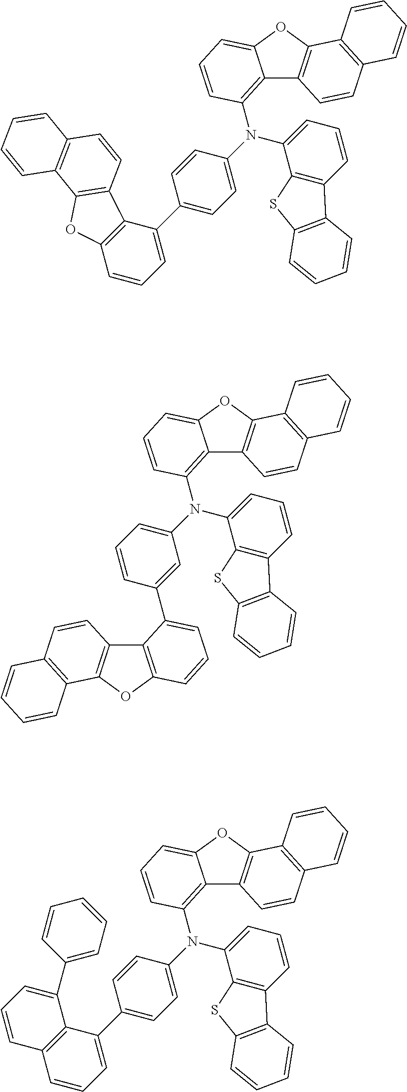 [SMILES: c1ccc2c(c1)ccc1c2oc2cccc(-c3ccc(N(c4cccc5c4sc4ccccc45)c4cccc5oc6c7ccccc7ccc6c45)cc3)c21 ; c1cc(-c2cccc3oc4c5ccccc5ccc4c23)cc(N(c2cccc3c2sc2ccccc23)c2cccc3oc4c5ccccc5ccc4c23)c1 ; c1ccc(-c2cccc3cccc(-c4ccc(N(c5cccc6c5sc5ccccc56)c5cccc6oc7c8ccccc8ccc7c56)cc4)c23)cc1]

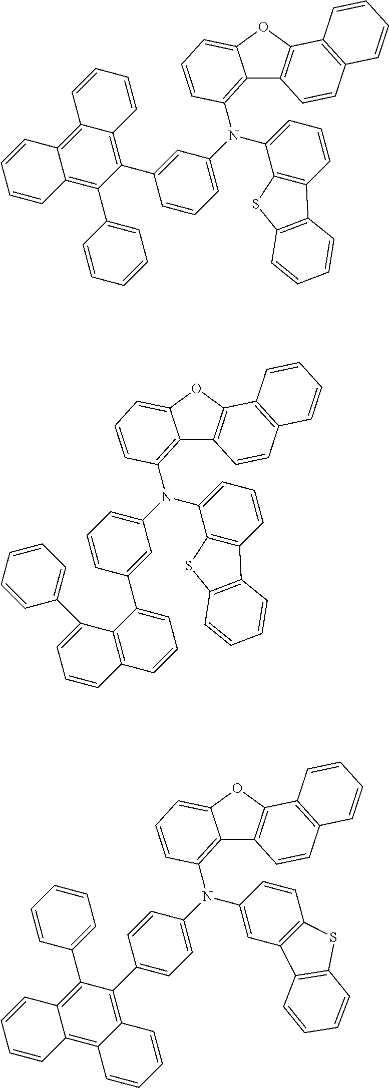 [SMILES: c1ccc(-c2c(-c3cccc(N(c4cccc5c4sc4ccccc45)c4cccc5oc6c7ccccc7ccc6c45)c3)c3ccccc3c3ccccc23)cc1 ; c1ccc(-c2cccc3cccc(-c4cccc(N(c5cccc6c5sc5ccccc56)c5cccc6oc7c8ccccc8ccc7c56)c4)c23)cc1 ; c1ccc(-c2c(-c3ccc(N(c4ccc5sc6ccccc6c5c4)c4cccc5oc6c7ccccc7ccc6c45)cc3)c3ccccc3c3ccccc23)cc1]

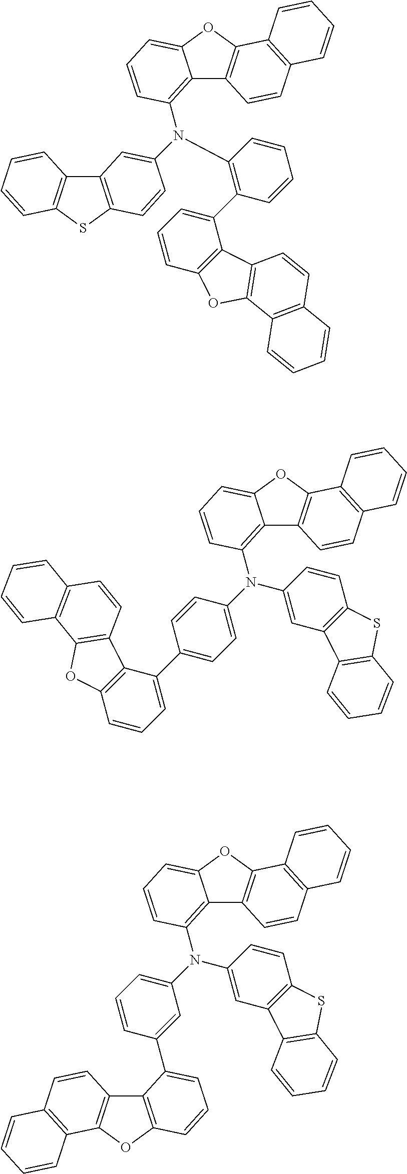 [SMILES: c1ccc(N(c2ccc3sc4ccccc4c3c2)c2cccc3oc4c5ccccc5ccc4c23)c(-c2cccc3oc4c5ccccc5ccc4c23)c1 ; c1ccc2c(c1)ccc1c2oc2cccc(-c3ccc(N(c4ccc5sc6ccccc6c5c4)c4cccc5oc6c7ccccc7ccc6c45)cc3)c21 ; c1cc(-c2cccc3oc4c5ccccc5ccc4c23)cc(N(c2ccc3sc4ccccc4c3c2)c2cccc3oc4c5ccccc5ccc4c23)c1]

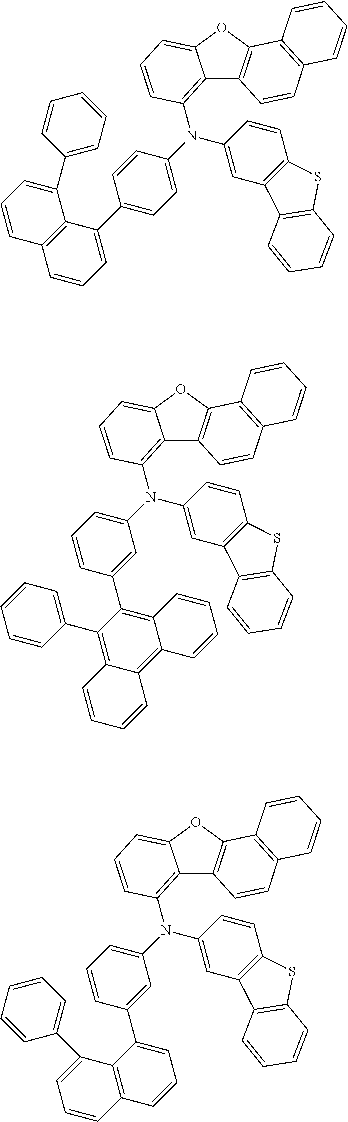 [SMILES: c1ccc(-c2cccc3cccc(-c4ccc(N(c5ccc6sc7ccccc7c6c5)c5cccc6oc7c8ccccc8ccc7c56)cc4)c23)cc1 ; c1ccc(-c2c(-c3cccc(N(c4ccc5sc6ccccc6c5c4)c4cccc5oc6c7ccccc7ccc6c45)c3)c3ccccc3c3ccccc23)cc1 ; c1ccc(-c2cccc3cccc(-c4cccc(N(c5ccc6sc7ccccc7c6c5)c5cccc6oc7c8ccccc8ccc7c56)c4)c23)cc1]

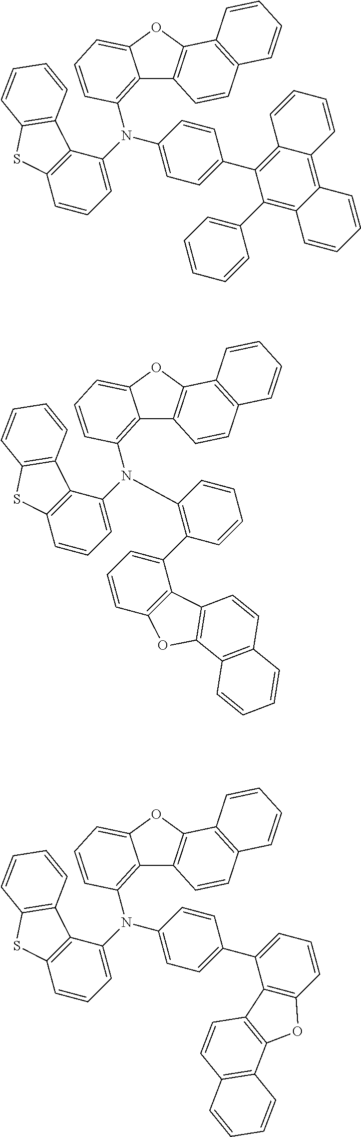 [SMILES: c1ccc(-c2c(-c3ccc(N(c4cccc5oc6c7ccccc7ccc6c45)c4cccc5sc6ccccc6c45)cc3)c3ccccc3c3ccccc23)cc1 ; c1ccc(N(c2cccc3oc4c5ccccc5ccc4c23)c2cccc3sc4ccccc4c23)c(-c2cccc3oc4c5ccccc5ccc4c23)c1 ; c1ccc2c(c1)ccc1c2oc2cccc(-c3ccc(N(c4cccc5oc6c7ccccc7ccc6c45)c4cccc5sc6ccccc6c45)cc3)c21]

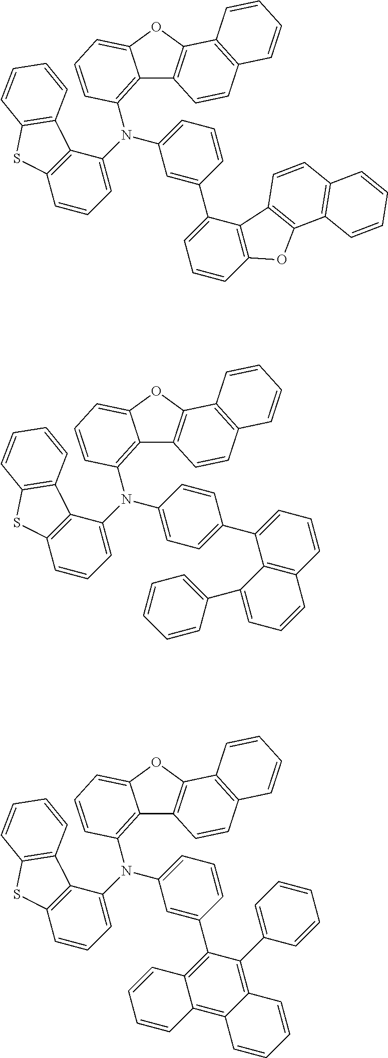 [SMILES: c1cc(-c2cccc3oc4c5ccccc5ccc4c23)cc(N(c2cccc3oc4c5ccccc5ccc4c23)c2cccc3sc4ccccc4c23)c1 ; c1ccc(-c2cccc3cccc(-c4ccc(N(c5cccc6oc7c8ccccc8ccc7c56)c5cccc6sc7ccccc7c56)cc4)c23)cc1 ; c1ccc(-c2c(-c3cccc(N(c4cccc5oc6c7ccccc7ccc6c45)c4cccc5sc6ccccc6c45)c3)c3ccccc3c3ccccc23)cc1]

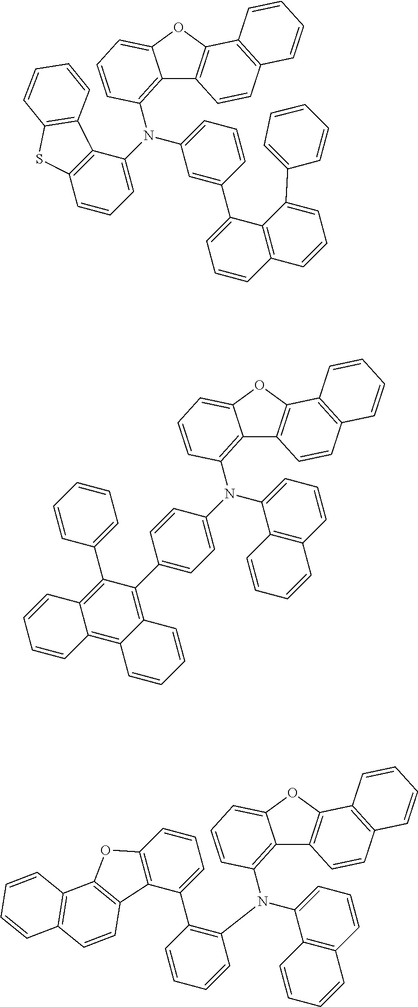 [SMILES: c1ccc(-c2cccc3cccc(-c4cccc(N(c5cccc6oc7c8ccccc8ccc7c56)c5cccc6sc7ccccc7c56)c4)c23)cc1 ; c1ccc(-c2c(-c3ccc(N(c4cccc5ccccc45)c4cccc5oc6c7ccccc7ccc6c45)cc3)c3ccccc3c3ccccc23)cc1 ; c1ccc(N(c2cccc3ccccc23)c2cccc3oc4c5ccccc5ccc4c23)c(-c2cccc3oc4c5ccccc5ccc4c23)c1]

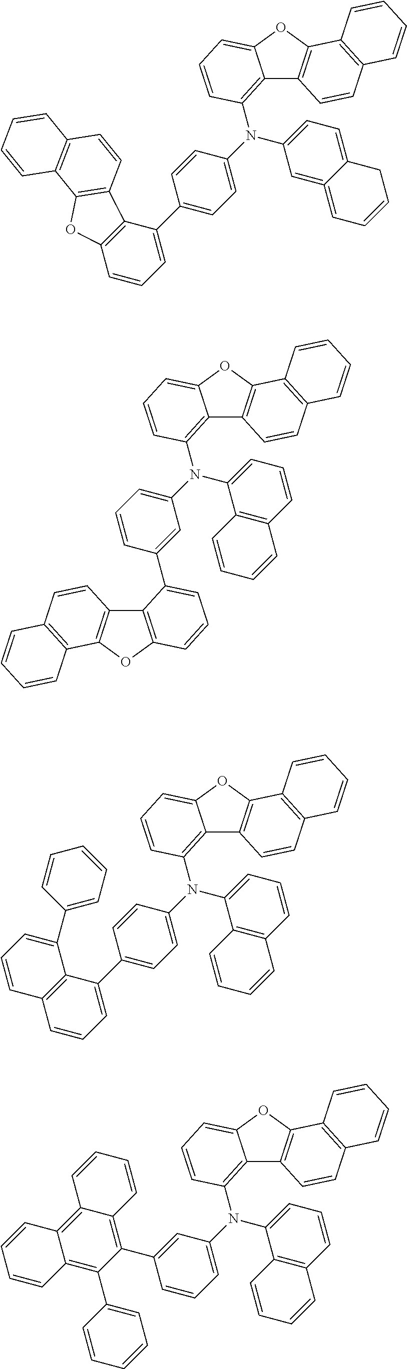

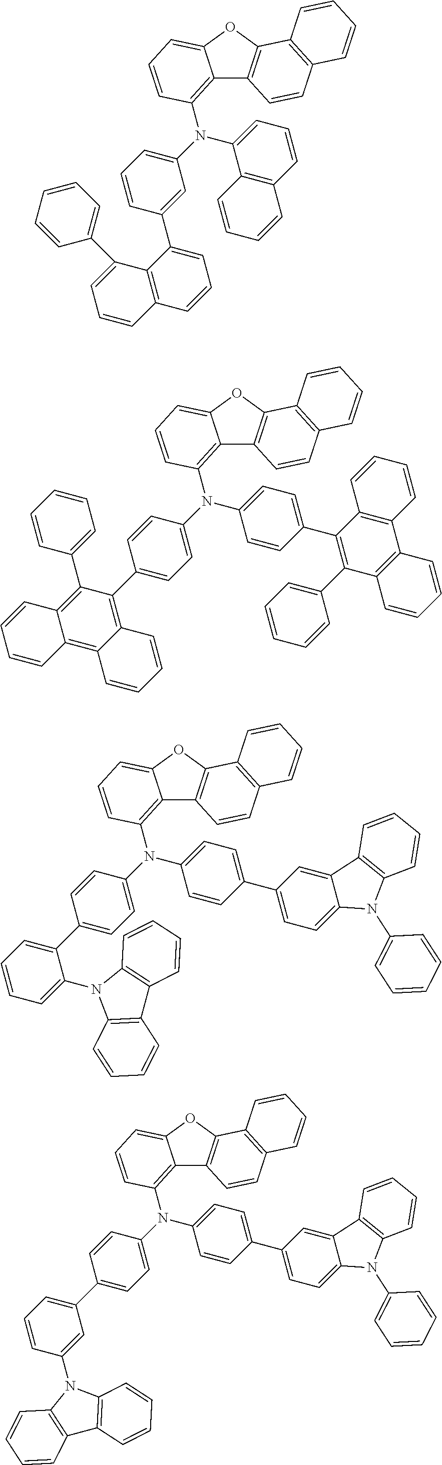 [SMILES: c1ccc(-c2cccc3cccc(-c4cccc(N(c5cccc6ccccc56)c5cccc6oc7c8ccccc8ccc7c56)c4)c23)cc1 ; c1ccc(-c2c(-c3ccc(N(c4ccc(-c5c(-c6ccccc6)c6ccccc6c6ccccc56)cc4)c4cccc5oc6c7ccccc7ccc6c45)cc3)c3ccccc3c3ccccc23)cc1 ; c1ccc(-n2c3ccccc3c3cc(-c4ccc(N(c5ccc(-c6ccccc6-n6c7ccccc7c7ccccc76)cc5)c5cccc6oc7c8ccccc8ccc7c56)cc4)ccc32)cc1 ; c1ccc(-n2c3ccccc3c3cc(-c4ccc(N(c5ccc(-c6cccc(-n7c8ccccc8c8ccccc87)c6)cc5)c5cccc6oc7c8ccccc8ccc7c56)cc4)ccc32)cc1]

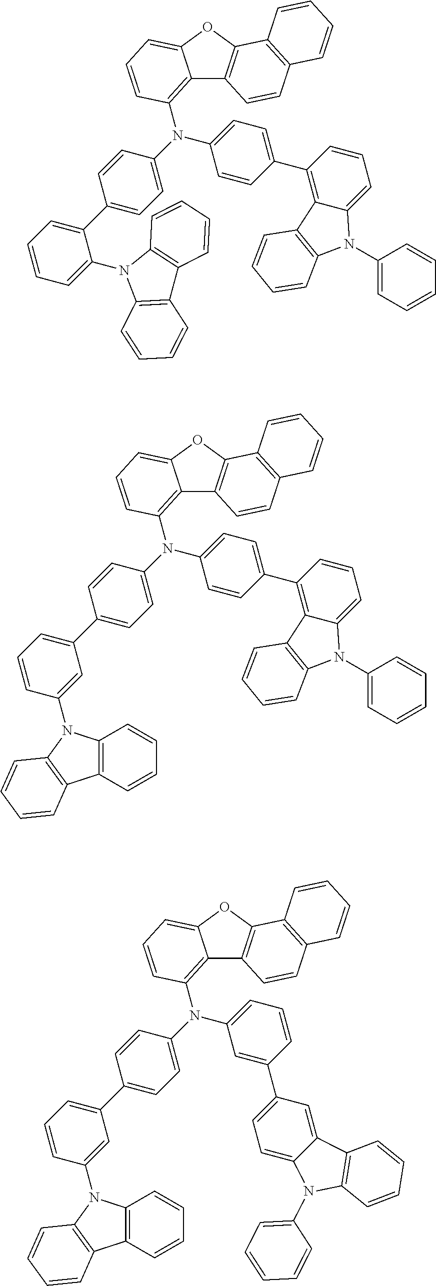 [SMILES: c1ccc(-n2c3ccccc3c3c(-c4ccc(N(c5ccc(-c6ccccc6-n6c7ccccc7c7ccccc76)cc5)c5cccc6oc7c8ccccc8ccc7c56)cc4)cccc32)cc1 ; c1ccc(-n2c3ccccc3c3c(-c4ccc(N(c5ccc(-c6cccc(-n7c8ccccc8c8ccccc87)c6)cc5)c5cccc6oc7c8ccccc8ccc7c56)cc4)cccc32)cc1 ; c1ccc(-n2c3ccccc3c3cc(-c4cccc(N(c5ccc(-c6cccc(-n7c8ccccc8c8ccccc87)c6)cc5)c5cccc6oc7c8ccccc8ccc7c56)c4)ccc32)cc1]

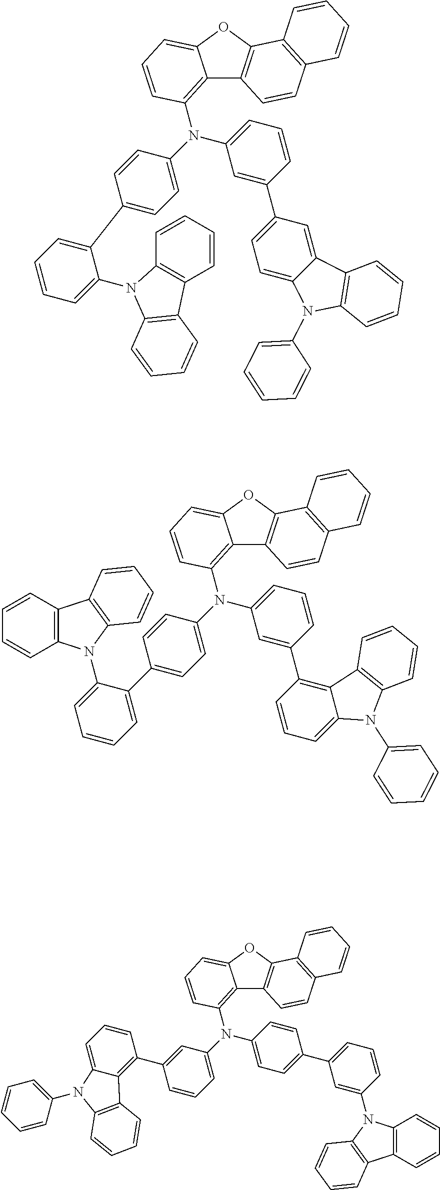 [SMILES: c1ccc(-n2c3ccccc3c3cc(-c4cccc(N(c5ccc(-c6ccccc6-n6c7ccccc7c7ccccc76)cc5)c5cccc6oc7c8ccccc8ccc7c56)c4)ccc32)cc1 ; c1ccc(-n2c3ccccc3c3c(-c4cccc(N(c5ccc(-c6ccccc6-n6c7ccccc7c7ccccc76)cc5)c5cccc6oc7c8ccccc8ccc7c56)c4)cccc32)cc1 ; c1ccc(-n2c3ccccc3c3c(-c4cccc(N(c5ccc(-c6cccc(-n7c8ccccc8c8ccccc87)c6)cc5)c5cccc6oc7c8ccccc8ccc7c56)c4)cccc32)cc1]

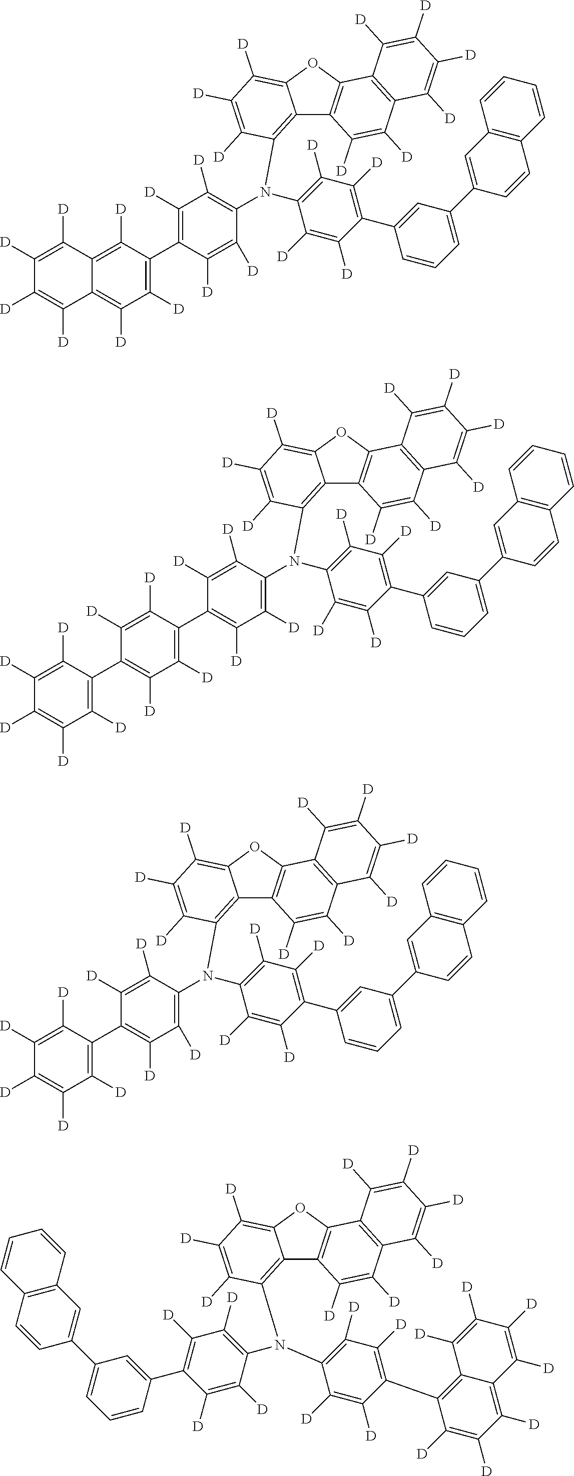

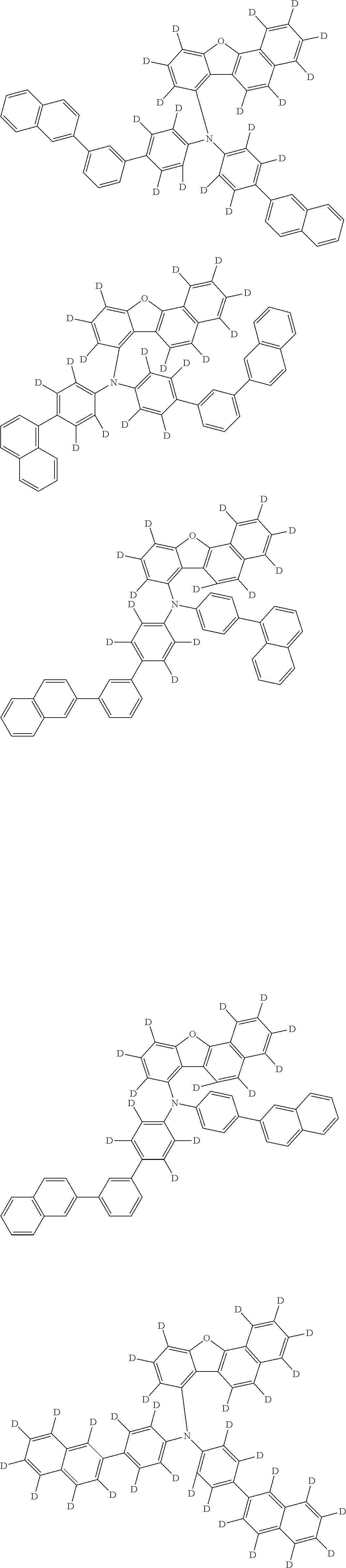

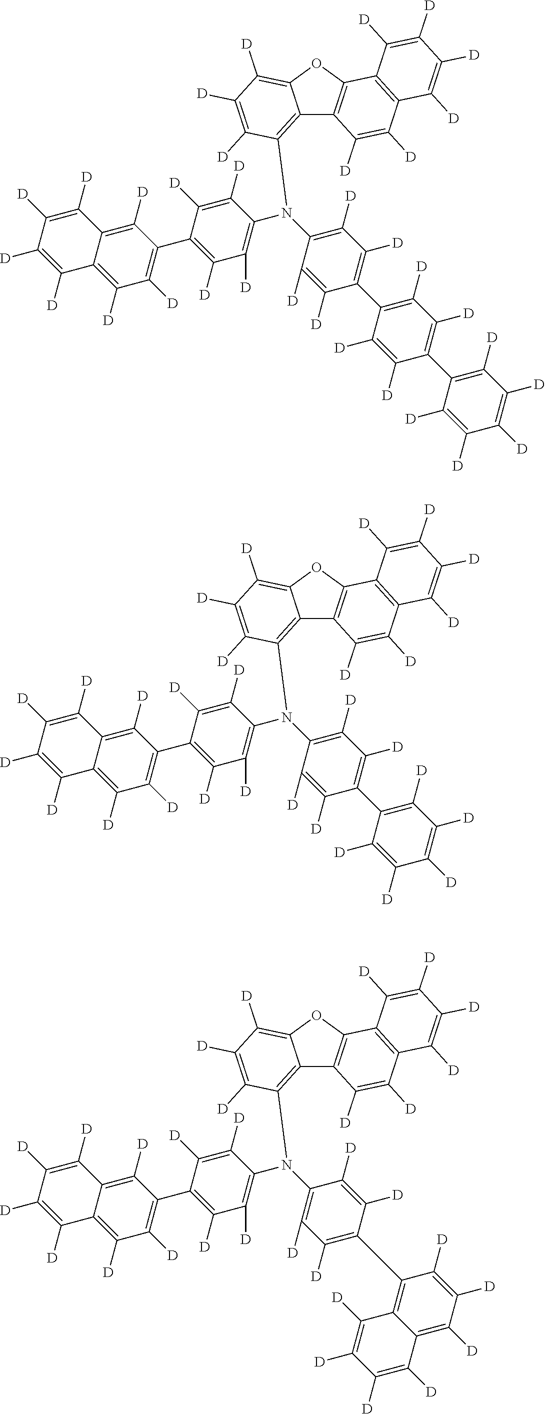

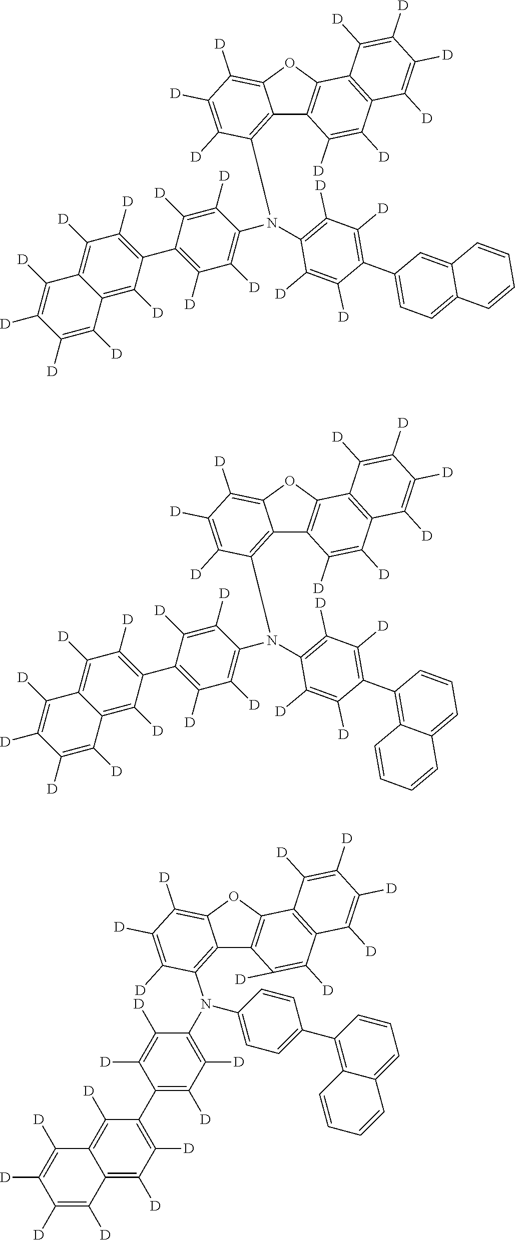

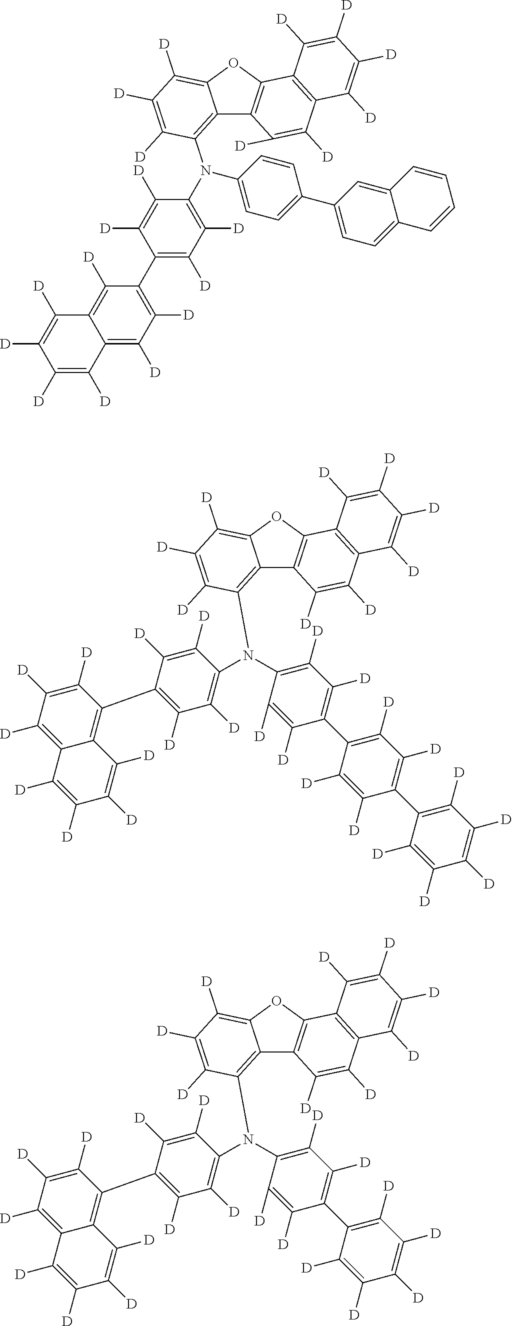

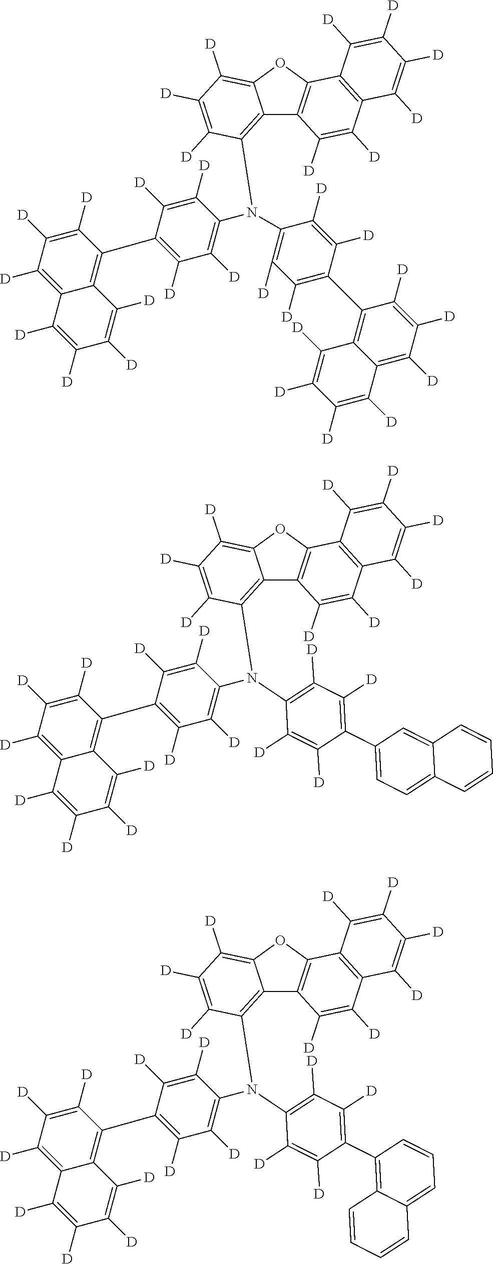

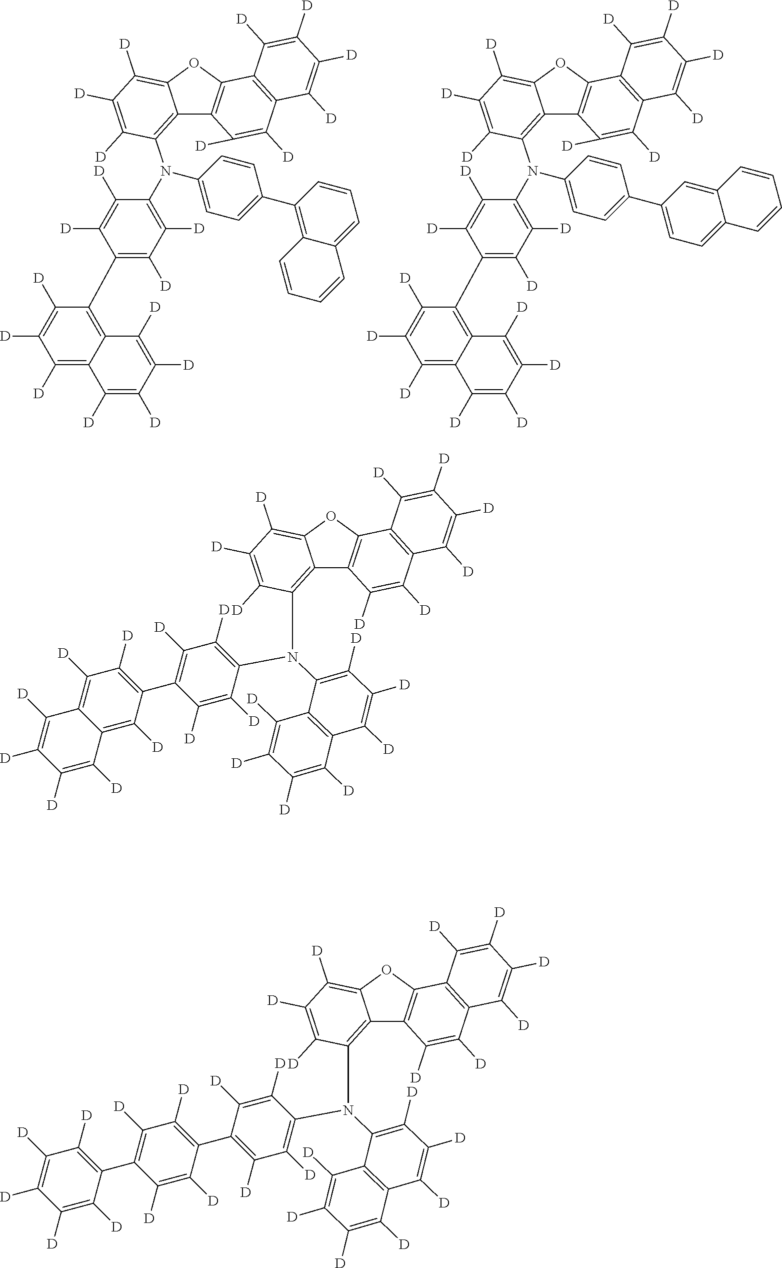

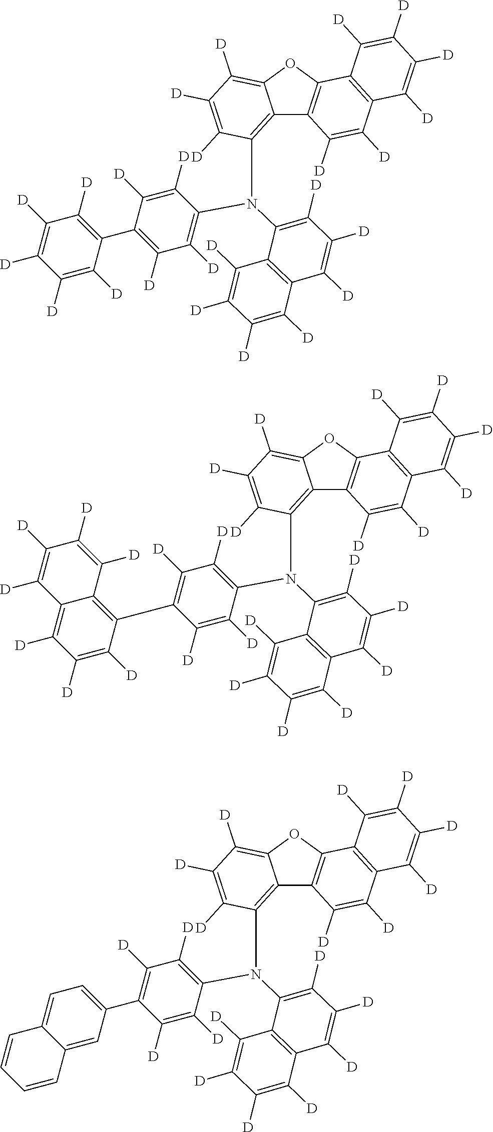

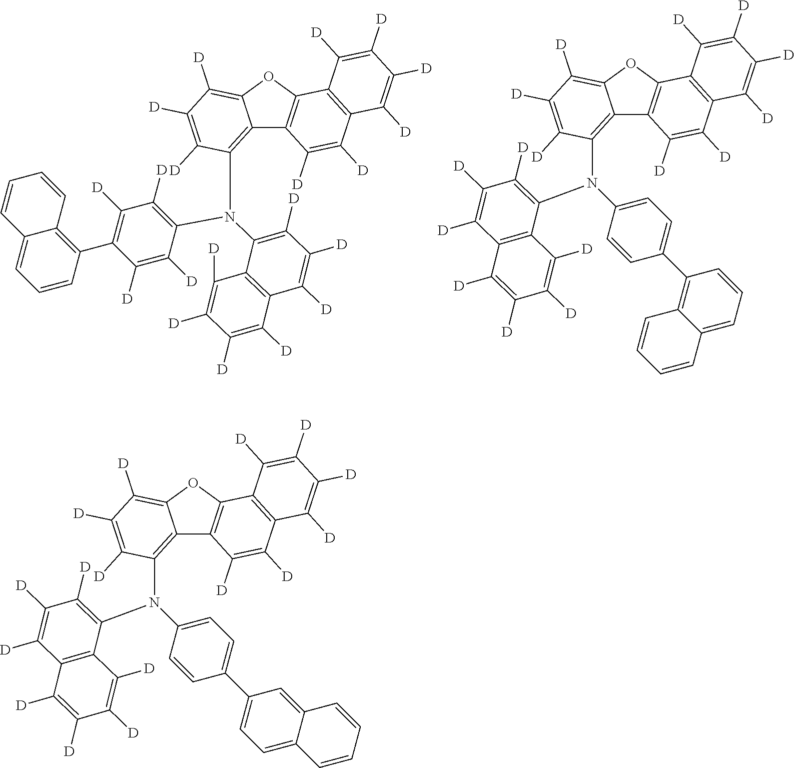

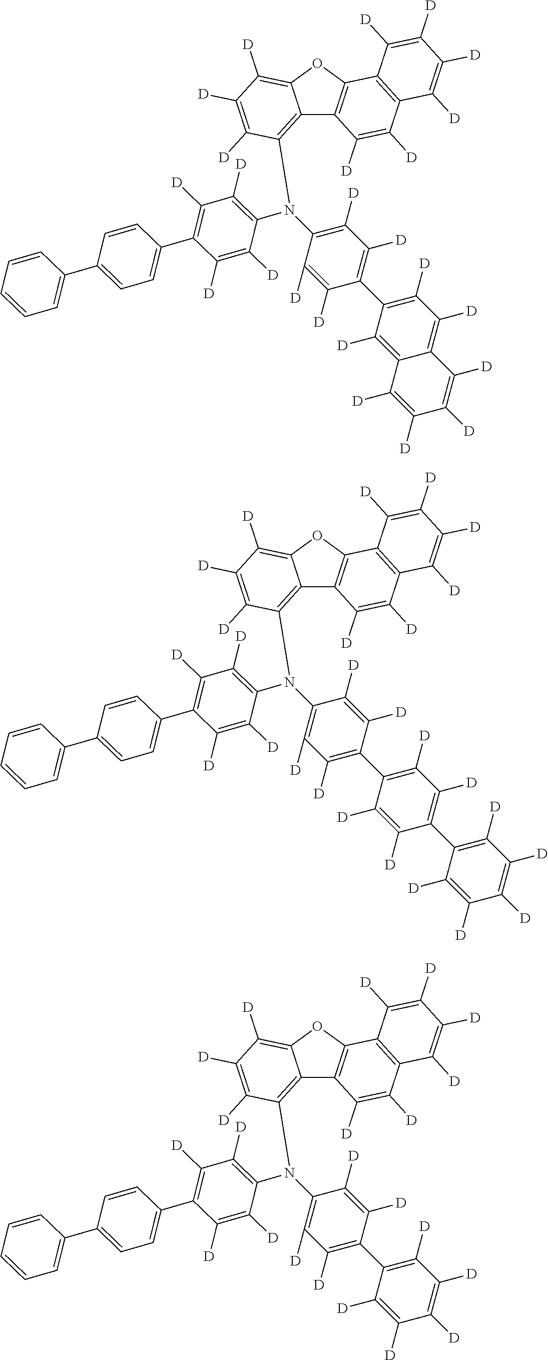

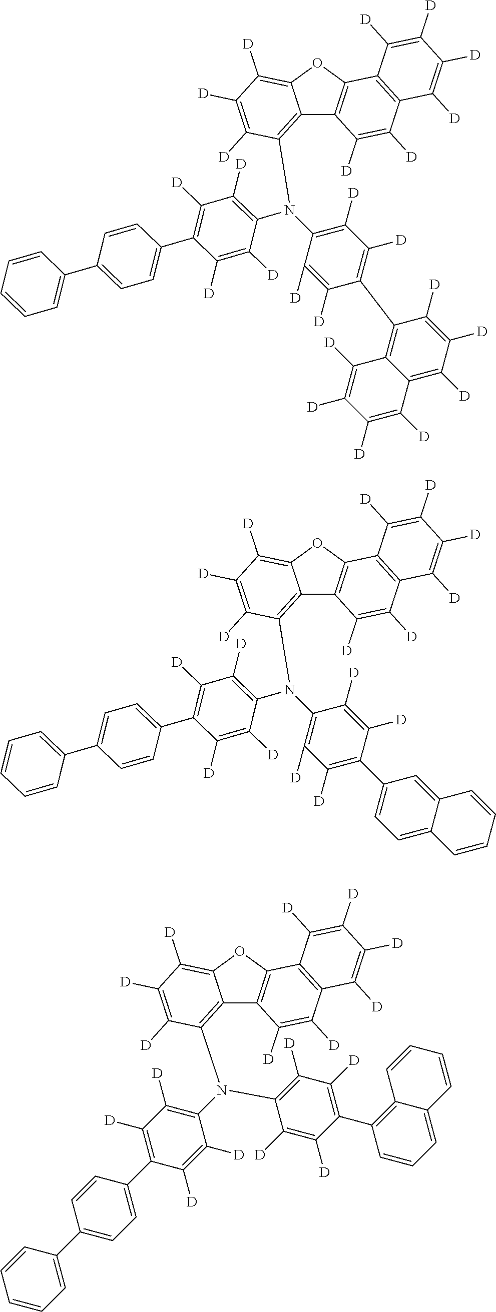

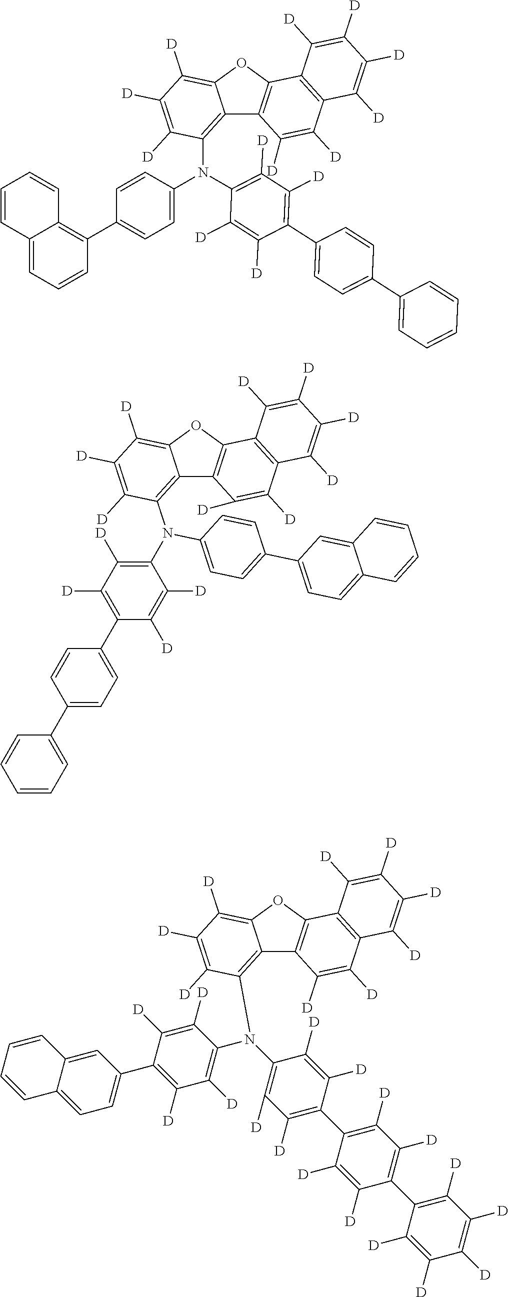

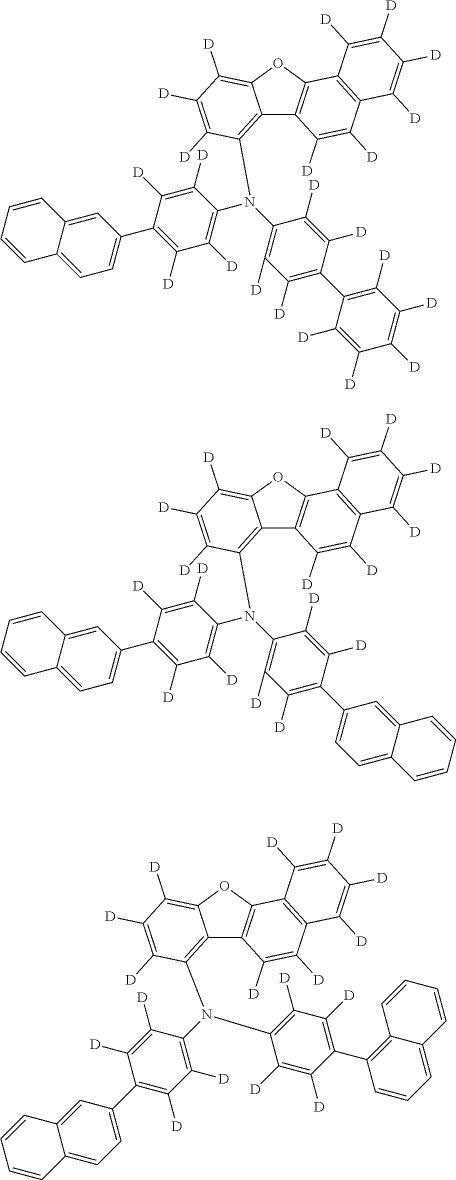

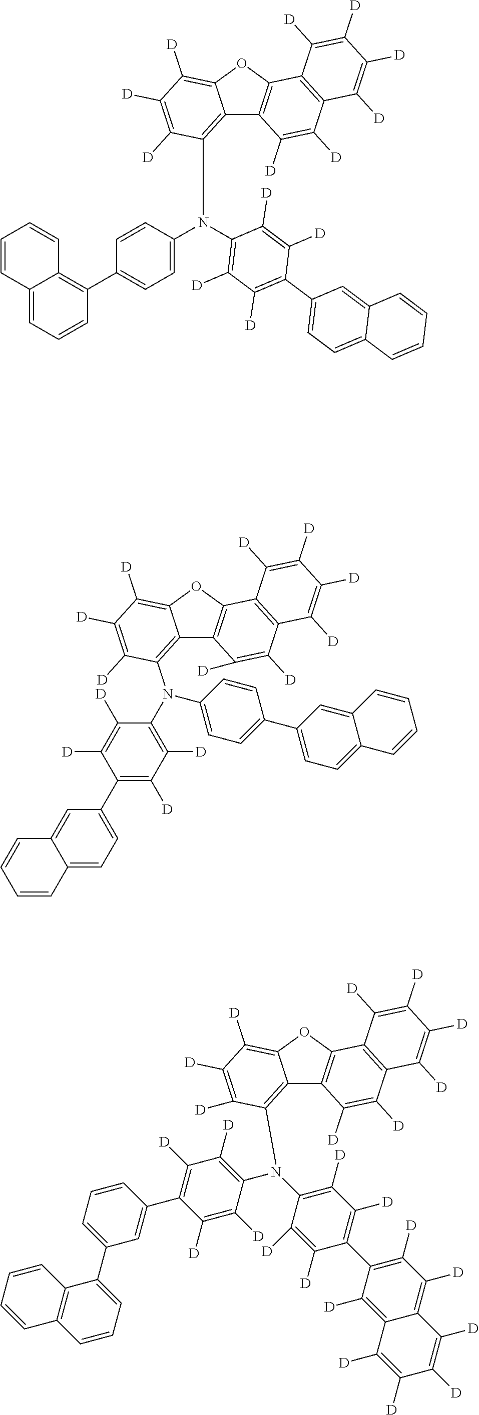

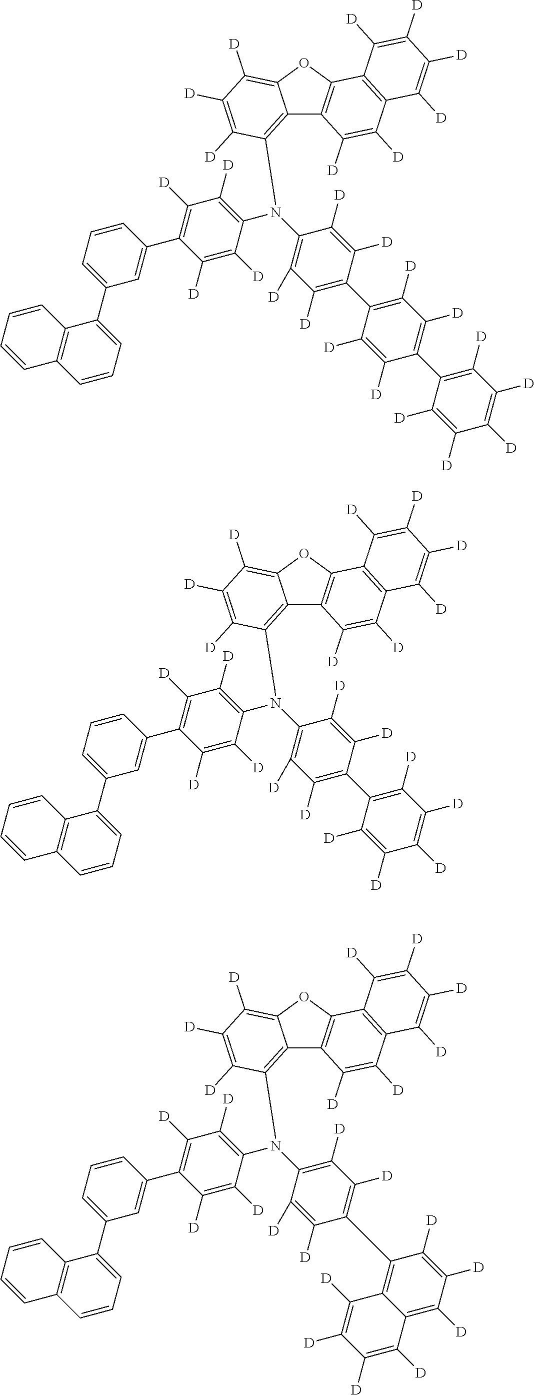

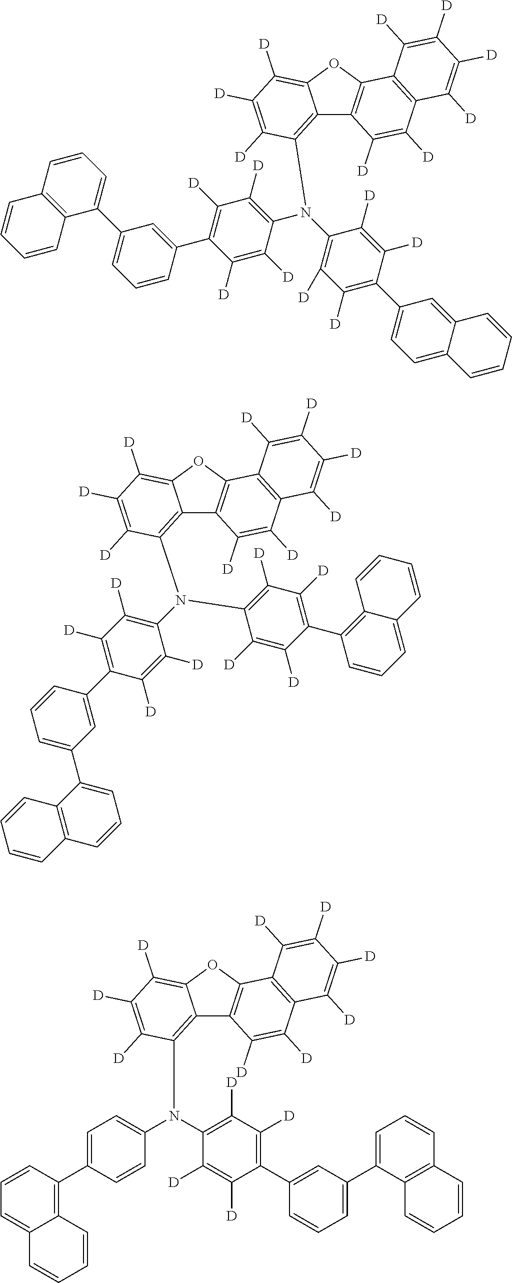

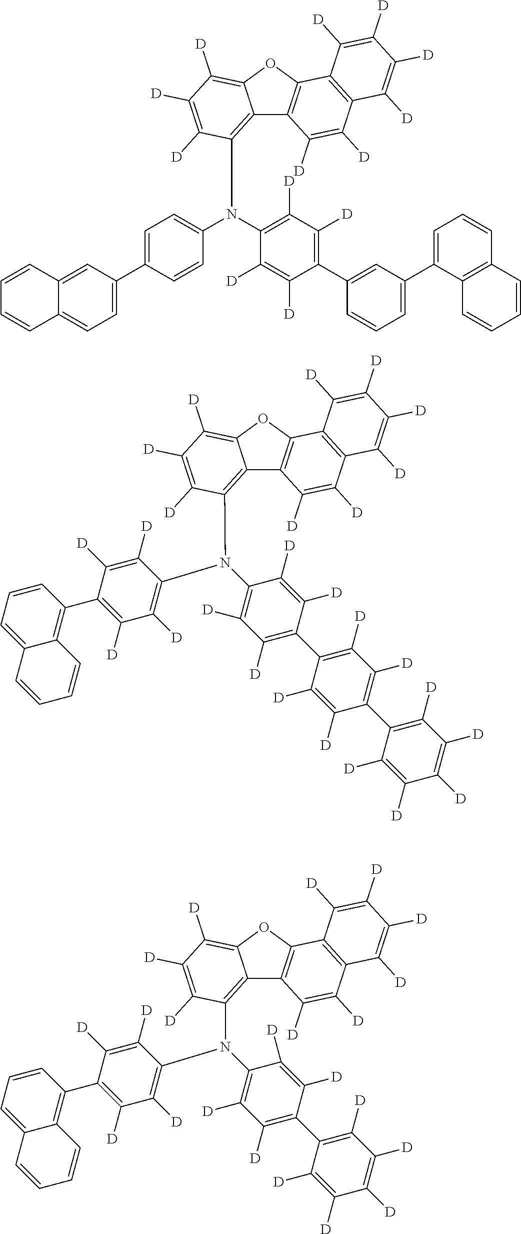

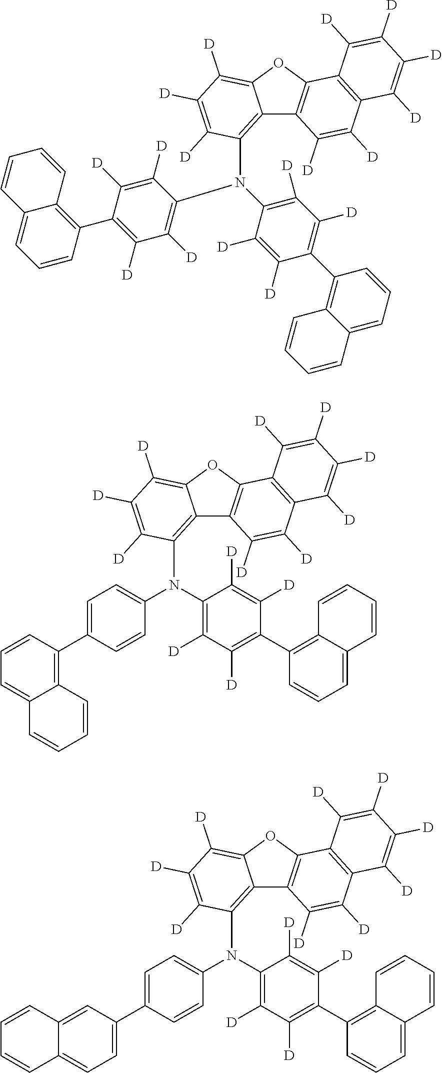

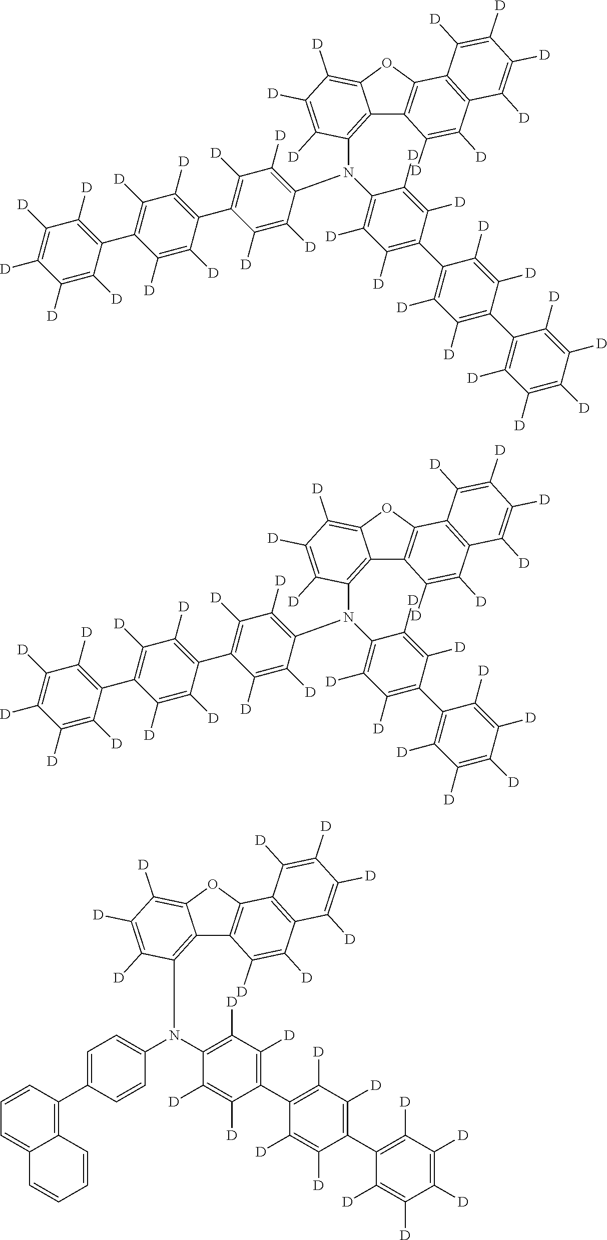

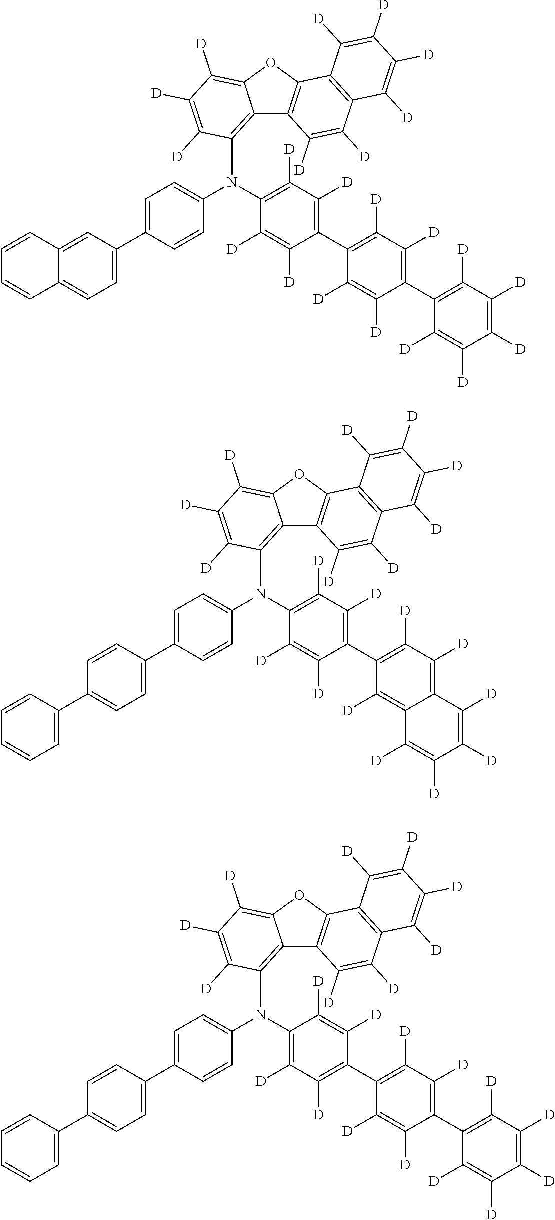

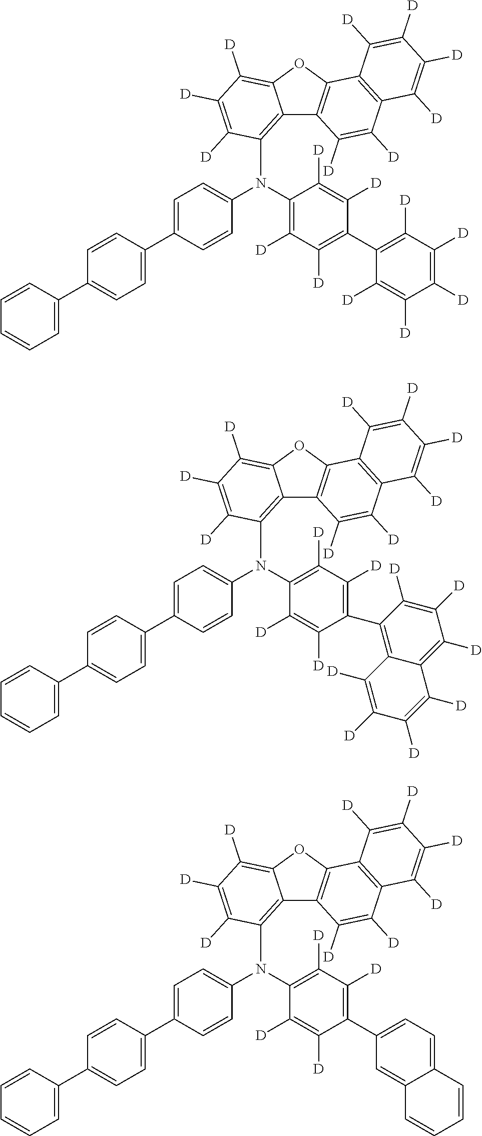

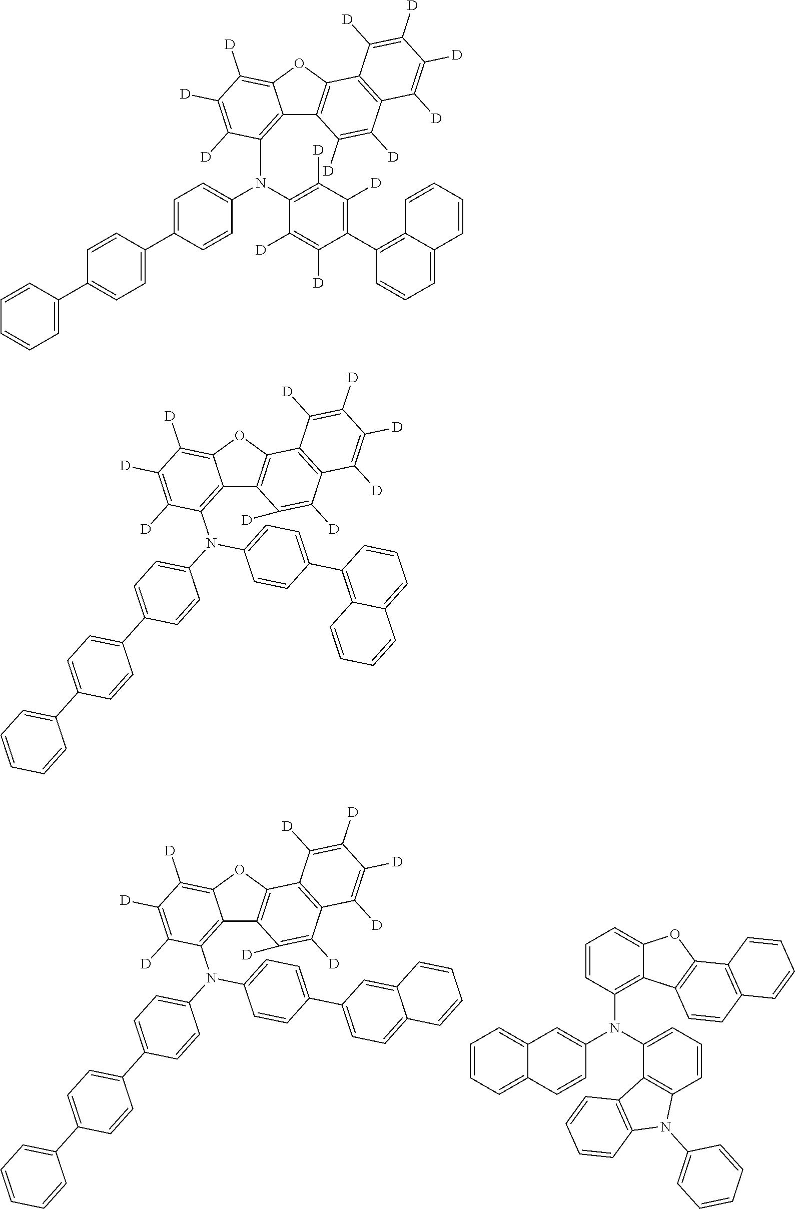

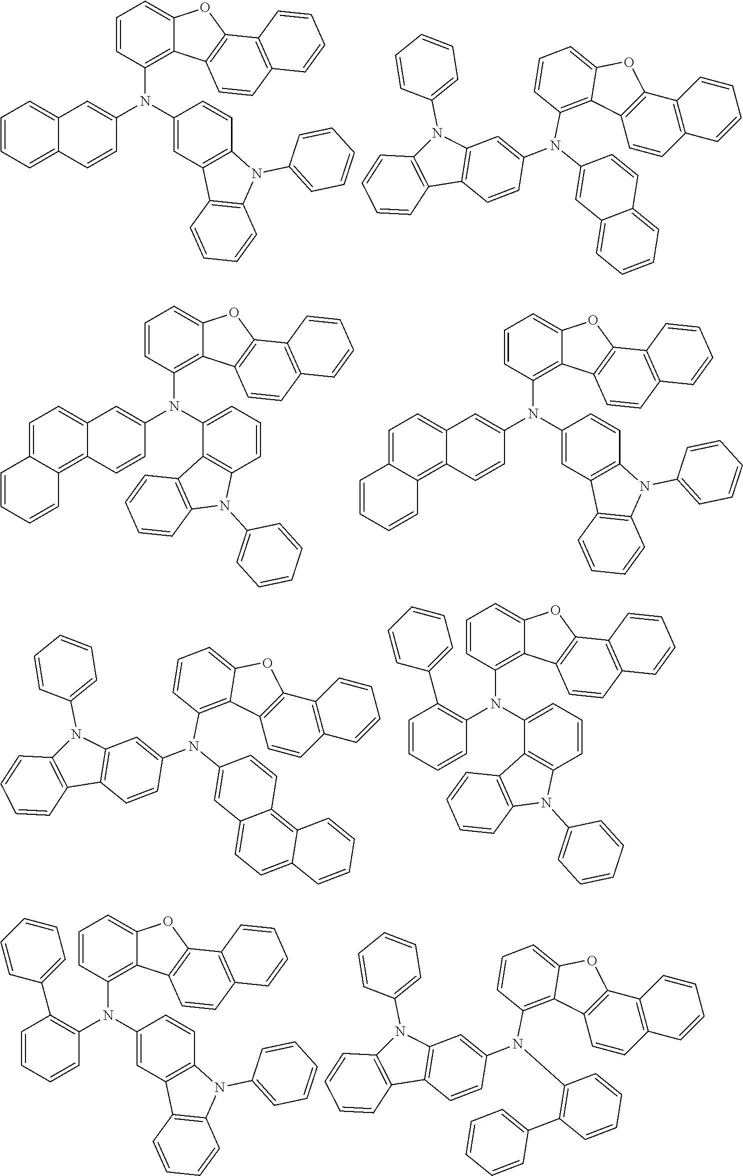

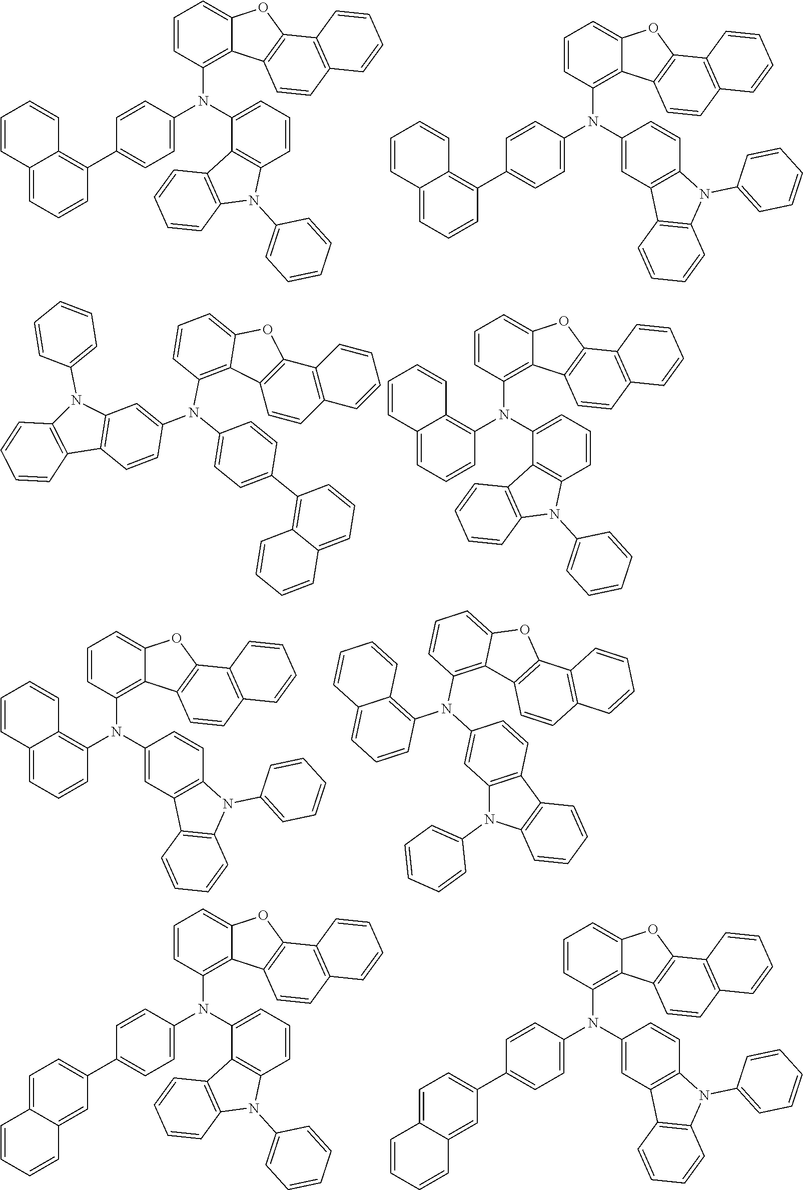

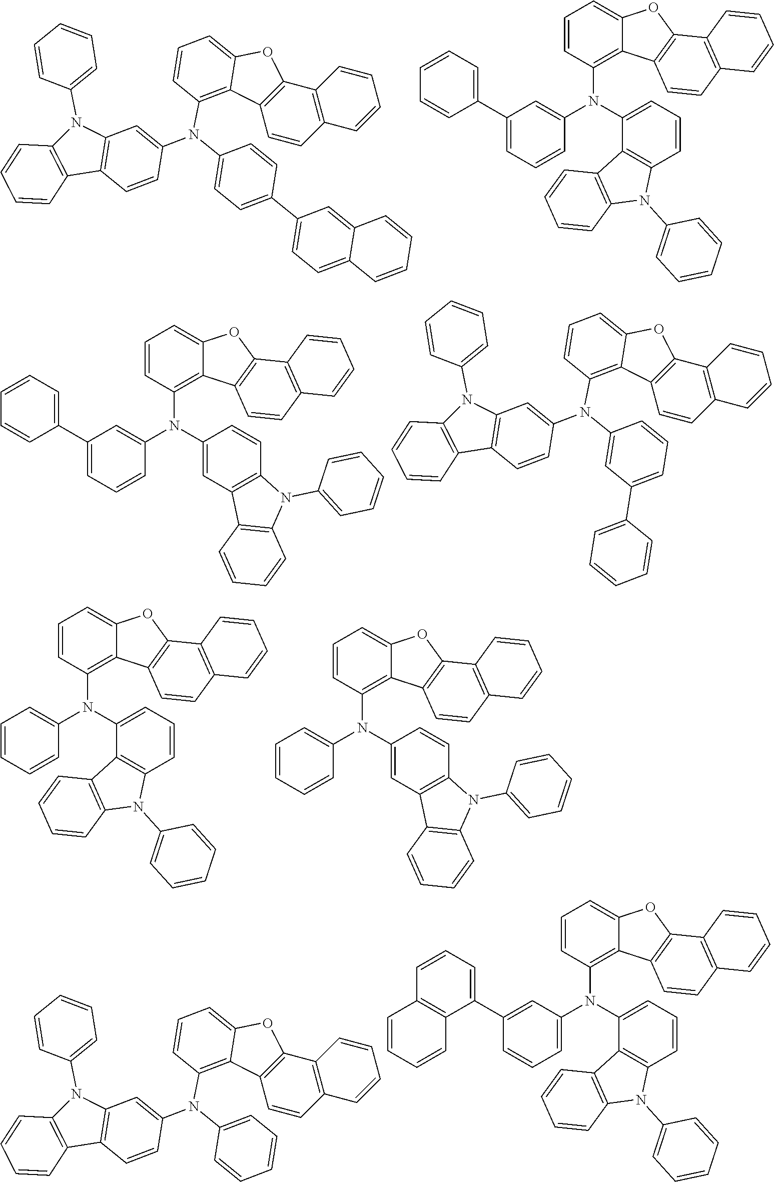

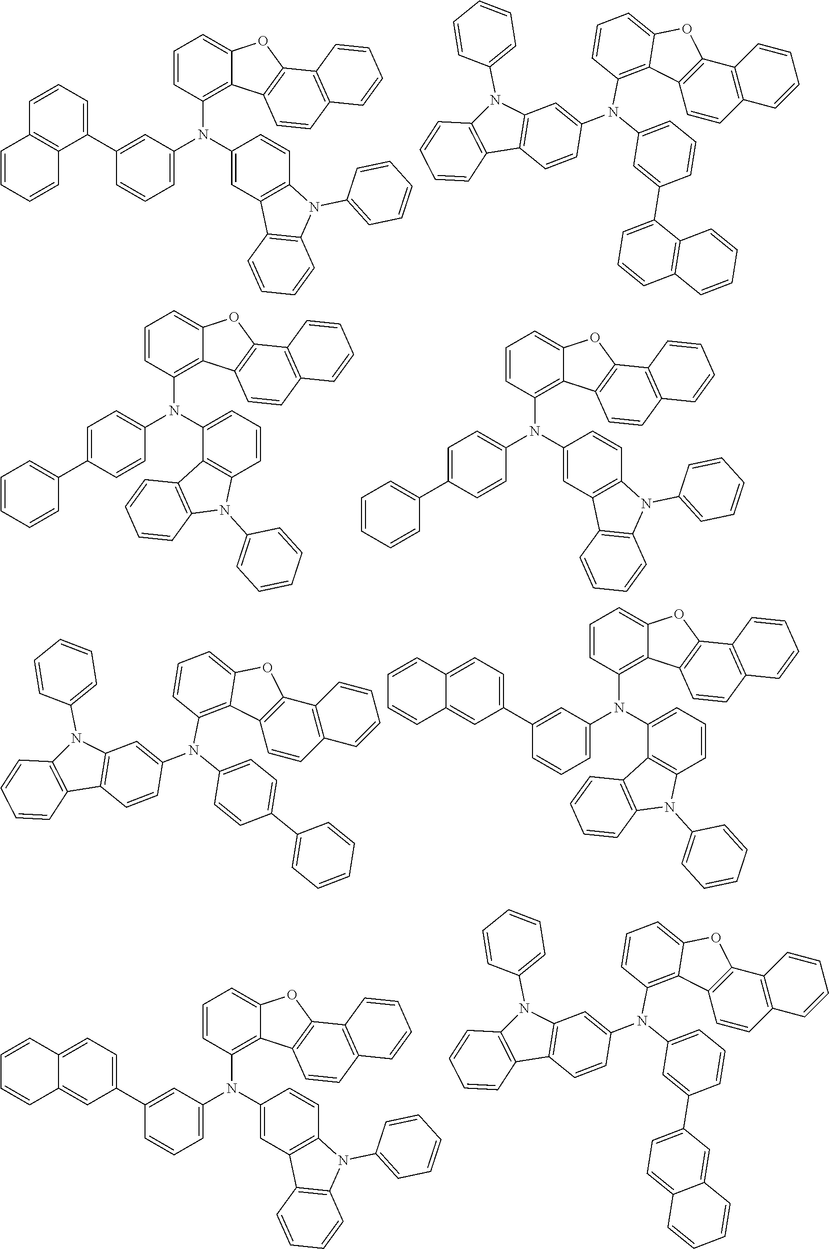

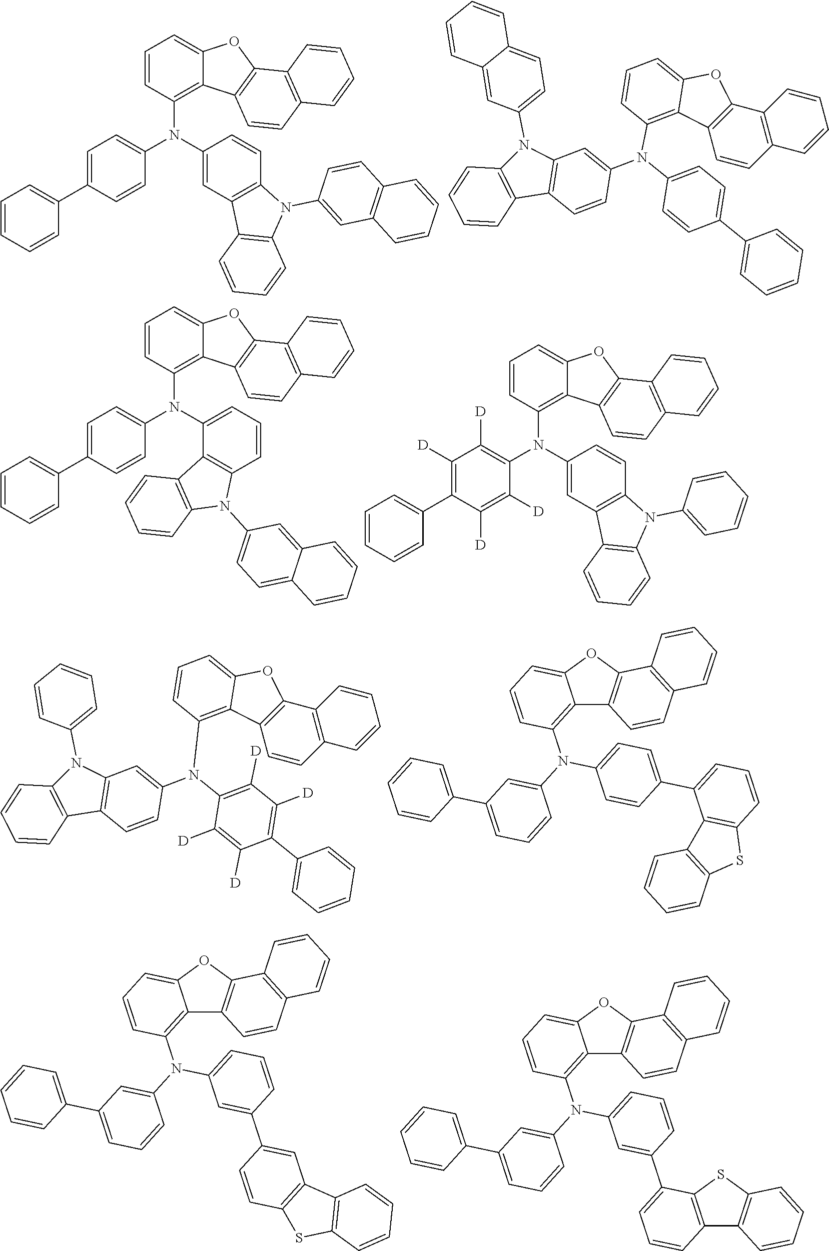

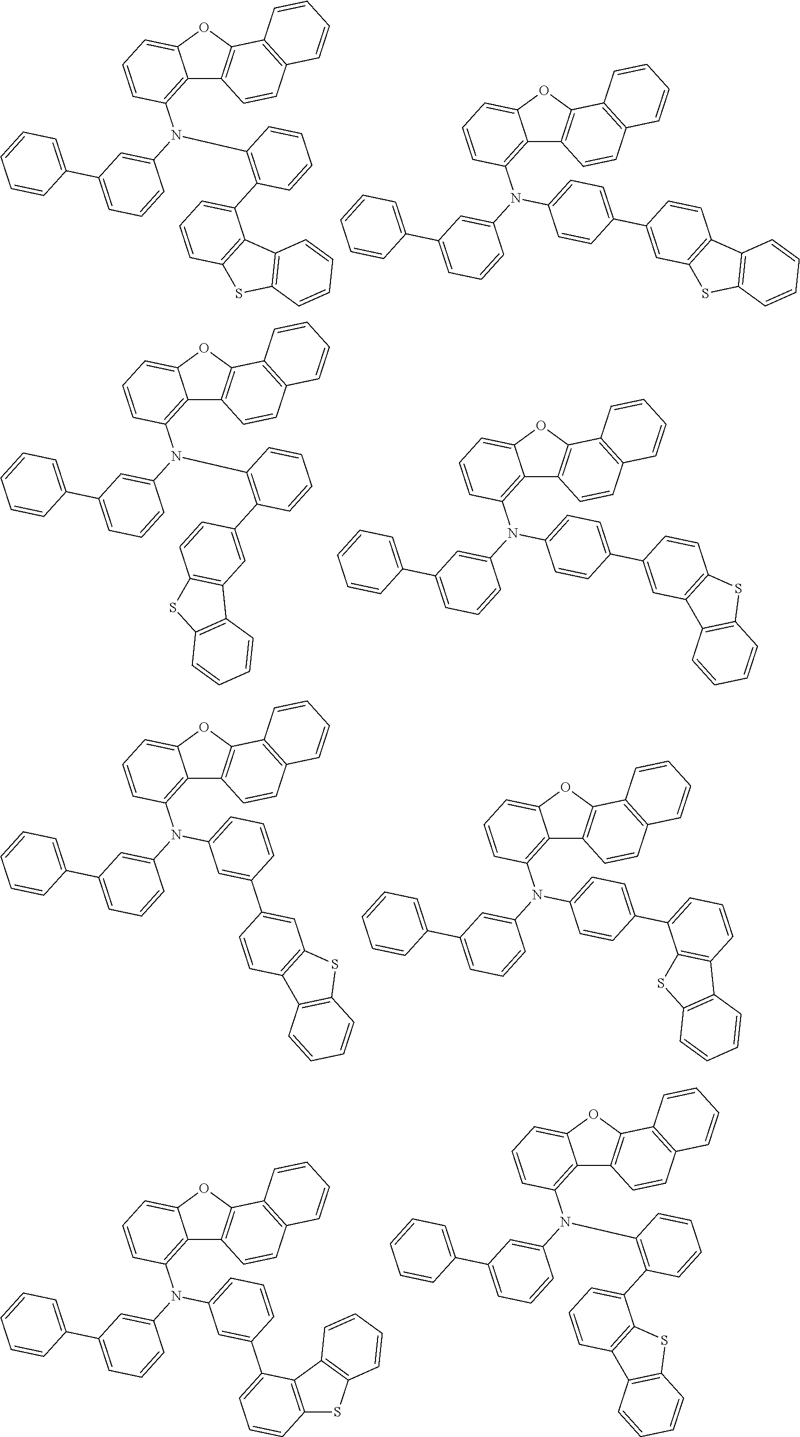

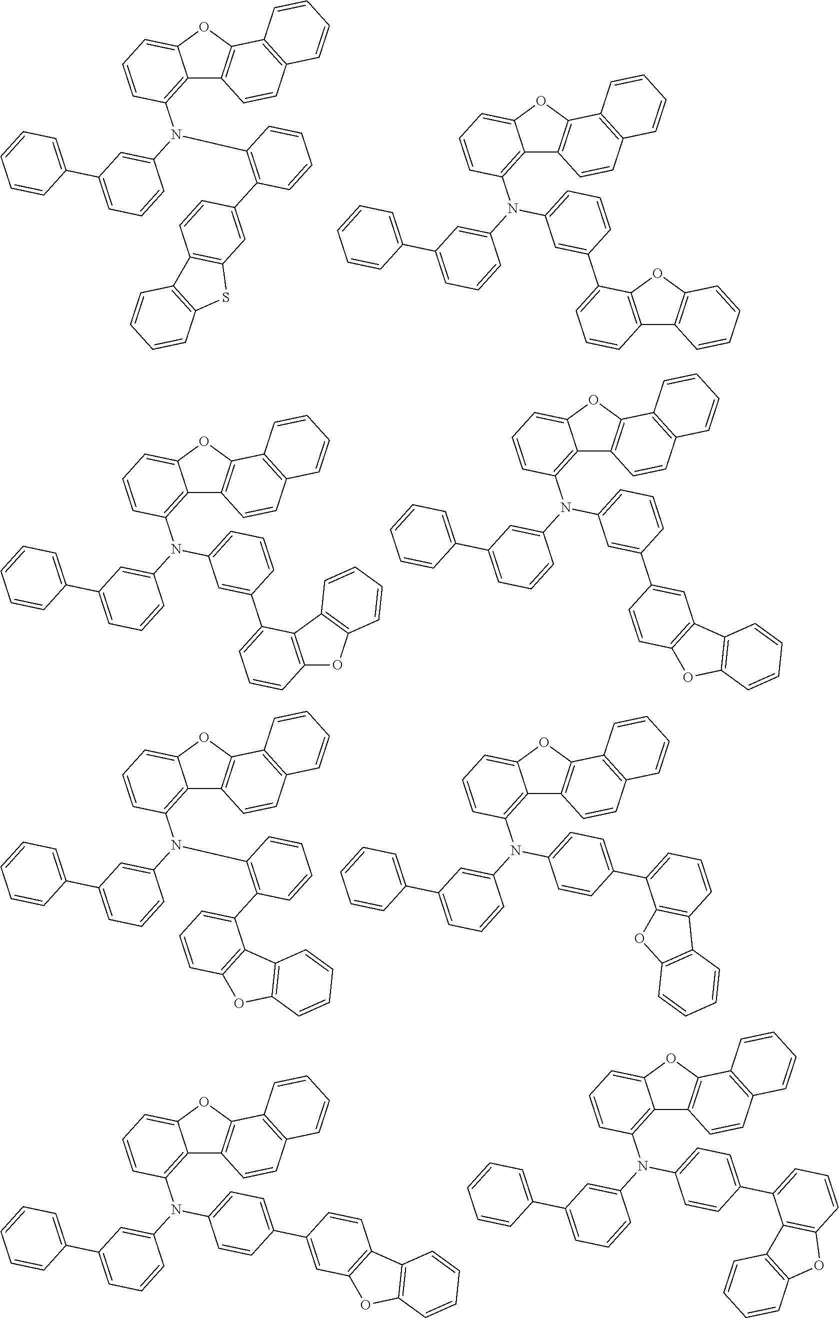

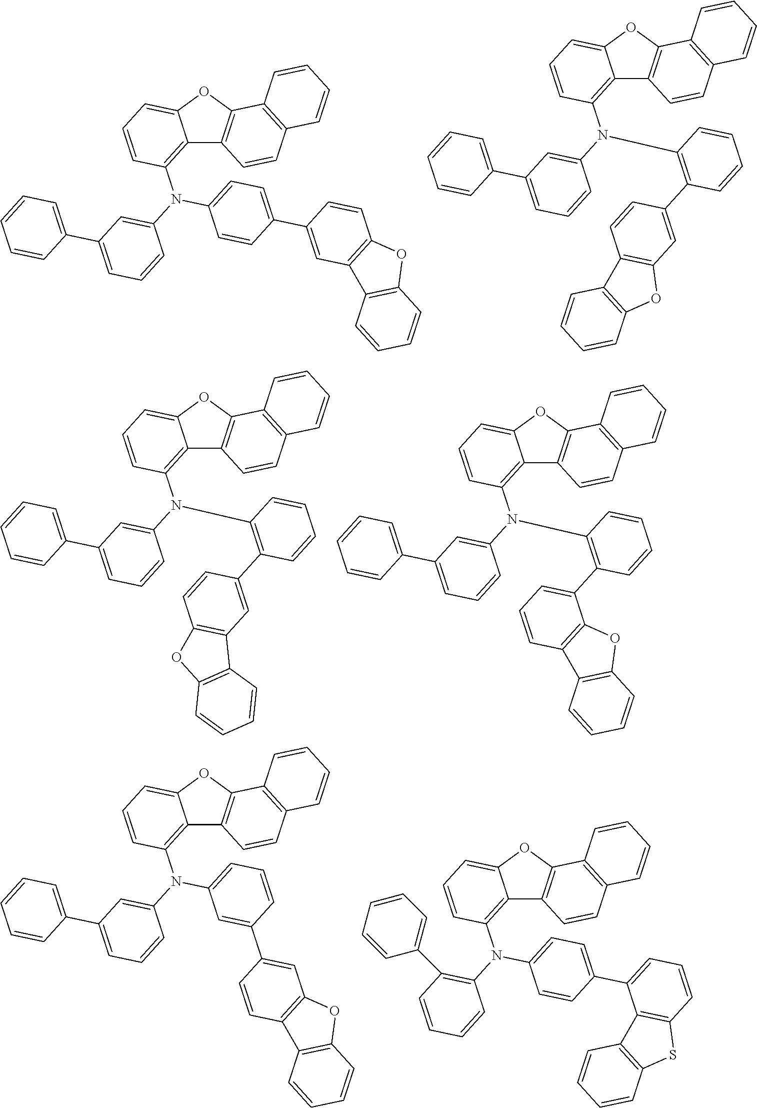

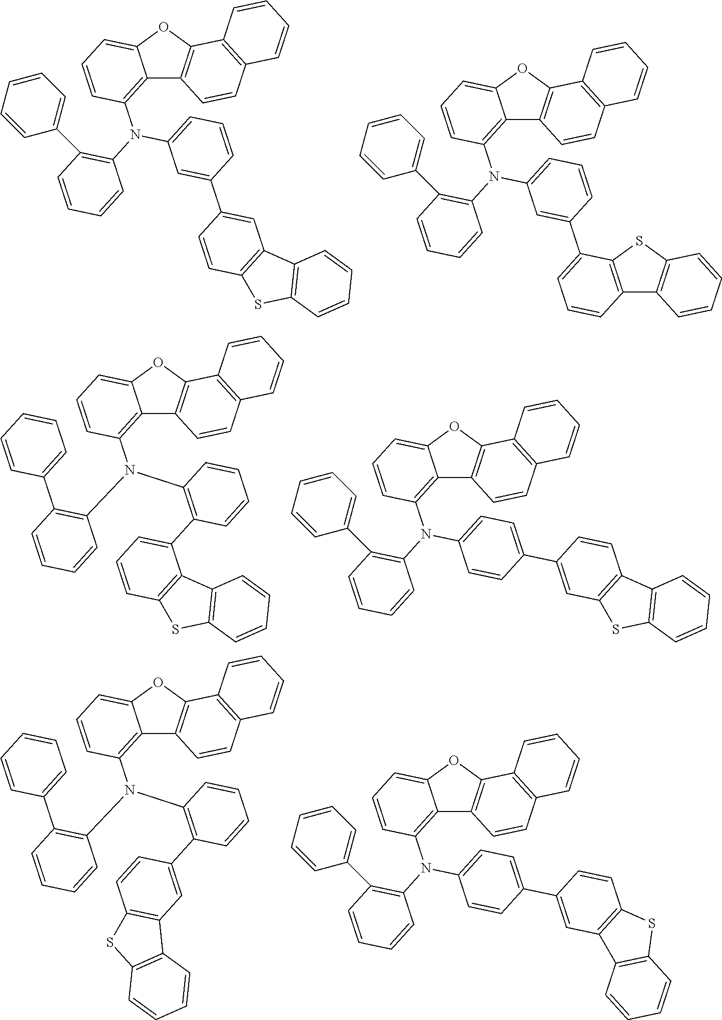

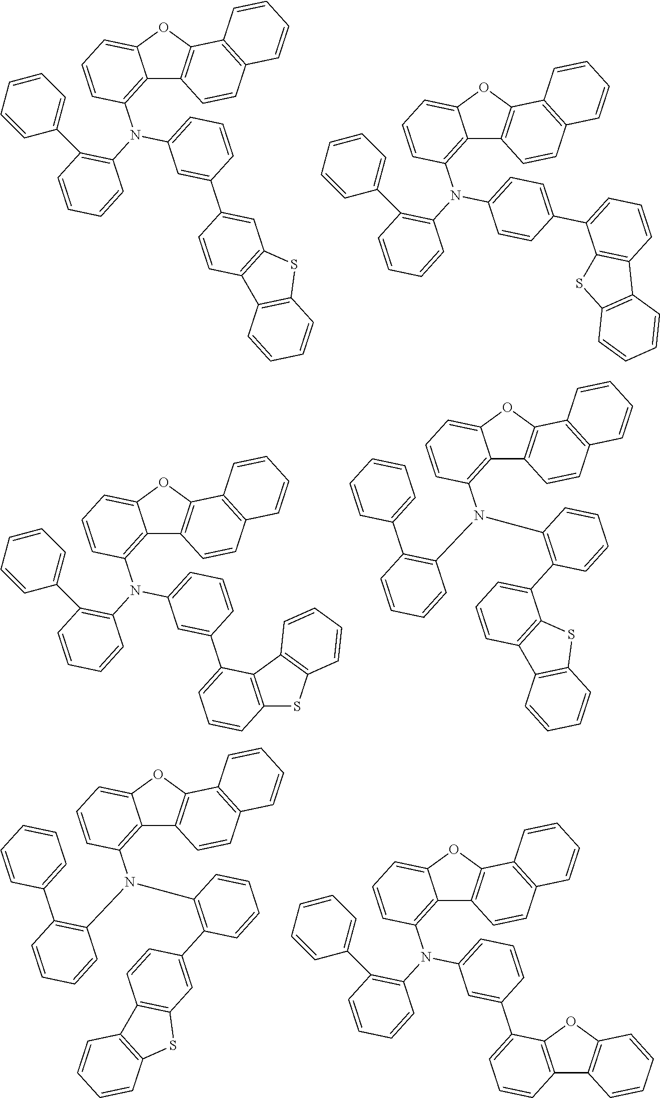

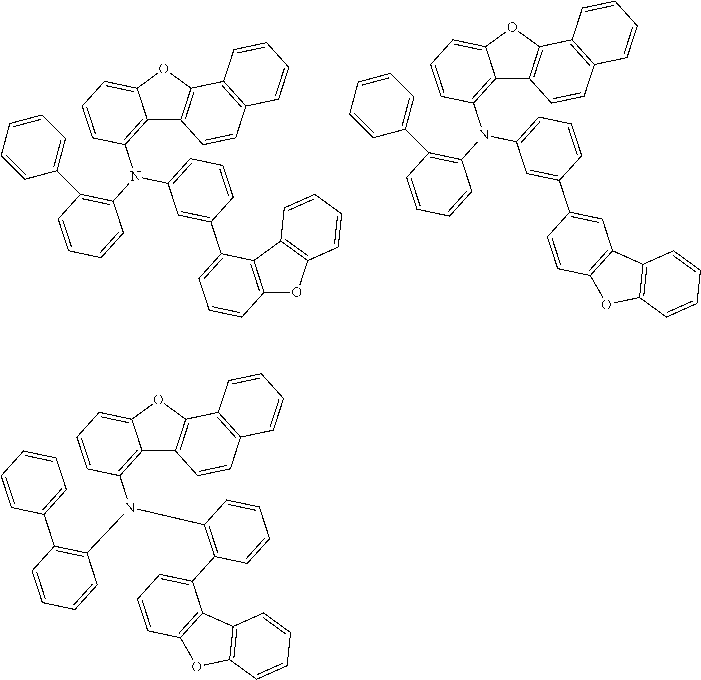 [SMILES: c1ccc(-c2ccccc2N(c2cccc(-c3cccc4oc5ccccc5c34)c2)c2cccc3oc4c5ccccc5ccc4c23)cc1 ; c1ccc(-c2ccccc2N(c2cccc(-c3ccc4oc5ccccc5c4c3)c2)c2cccc3oc4c5ccccc5ccc4c23)cc1 ; c1ccc(-c2ccccc2N(c2ccccc2-c2cccc3oc4ccccc4c23)c2cccc3oc4c5ccccc5ccc4c23)cc1]

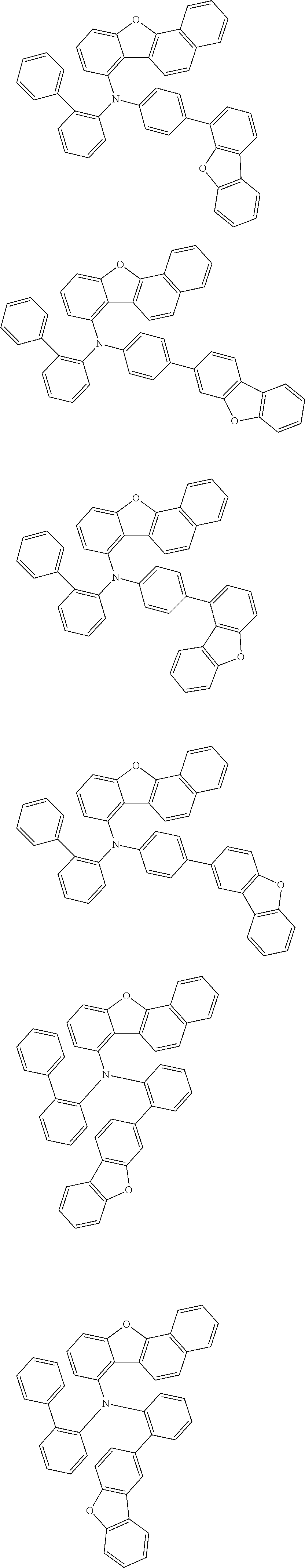 [SMILES: c1ccc(-c2ccccc2N(c2ccc(-c3cccc4c3oc3ccccc34)cc2)c2cccc3oc4c5ccccc5ccc4c23)cc1 ; c1ccc(-c2ccccc2N(c2ccc(-c3ccc4c(c3)oc3ccccc34)cc2)c2cccc3oc4c5ccccc5ccc4c23)cc1 ; c1ccc(-c2ccccc2N(c2ccc(-c3cccc4oc5ccccc5c34)cc2)c2cccc3oc4c5ccccc5ccc4c23)cc1 ; c1ccc(-c2ccccc2N(c2ccc(-c3ccc4oc5ccccc5c4c3)cc2)c2cccc3oc4c5ccccc5ccc4c23)cc1 ; c1ccc(-c2ccccc2N(c2ccccc2-c2ccc3c(c2)oc2ccccc23)c2cccc3oc4c5ccccc5ccc4c23)cc1 ; c1ccc(-c2ccccc2N(c2ccccc2-c2ccc3oc4ccccc4c3c2)c2cccc3oc4c5ccccc5ccc4c23)cc1]

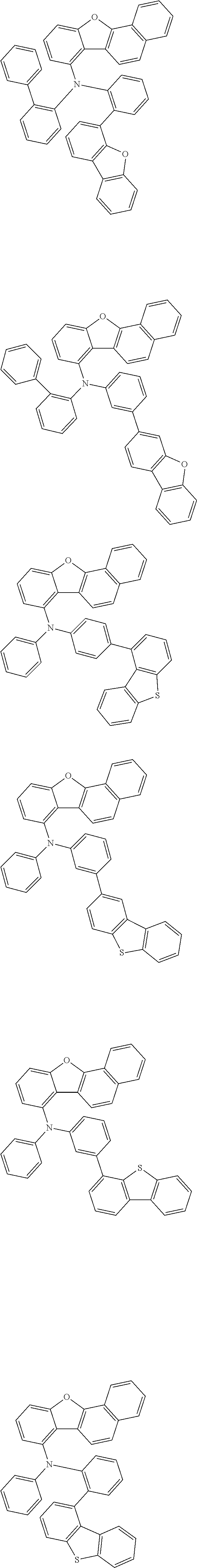 [SMILES: c1ccc(-c2ccccc2N(c2ccccc2-c2cccc3c2oc2ccccc23)c2cccc3oc4c5ccccc5ccc4c23)cc1 ; c1ccc(-c2ccccc2N(c2cccc(-c3ccc4c(c3)oc3ccccc34)c2)c2cccc3oc4c5ccccc5ccc4c23)cc1 ; c1ccc(N(c2ccc(-c3cccc4sc5ccccc5c34)cc2)c2cccc3oc4c5ccccc5ccc4c23)cc1 ; c1ccc(N(c2cccc(-c3ccc4sc5ccccc5c4c3)c2)c2cccc3oc4c5ccccc5ccc4c23)cc1 ; c1ccc(N(c2cccc(-c3cccc4c3sc3ccccc34)c2)c2cccc3oc4c5ccccc5ccc4c23)cc1 ; c1ccc(N(c2ccccc2-c2cccc3sc4ccccc4c23)c2cccc3oc4c5ccccc5ccc4c23)cc1]

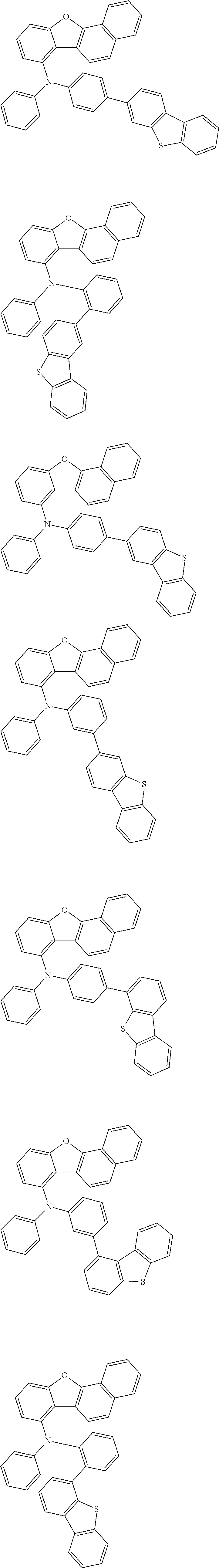

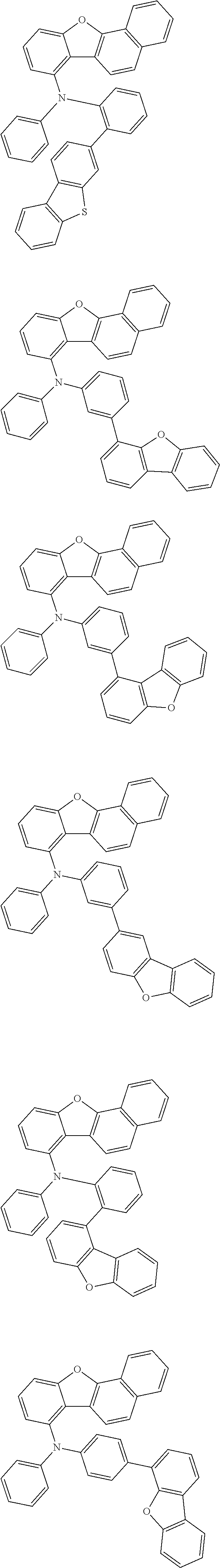 [SMILES: c1ccc(N(c2ccccc2-c2ccc3c(c2)sc2ccccc23)c2cccc3oc4c5ccccc5ccc4c23)cc1 ; c1ccc(N(c2cccc(-c3cccc4c3oc3ccccc34)c2)c2cccc3oc4c5ccccc5ccc4c23)cc1 ; c1ccc(N(c2cccc(-c3cccc4oc5ccccc5c34)c2)c2cccc3oc4c5ccccc5ccc4c23)cc1 ; c1ccc(N(c2cccc(-c3ccc4oc5ccccc5c4c3)c2)c2cccc3oc4c5ccccc5ccc4c23)cc1 ; c1ccc(N(c2ccccc2-c2cccc3oc4ccccc4c23)c2cccc3oc4c5ccccc5ccc4c23)cc1 ; c1ccc(N(c2ccc(-c3cccc4c3oc3ccccc34)cc2)c2cccc3oc4c5ccccc5ccc4c23)cc1]

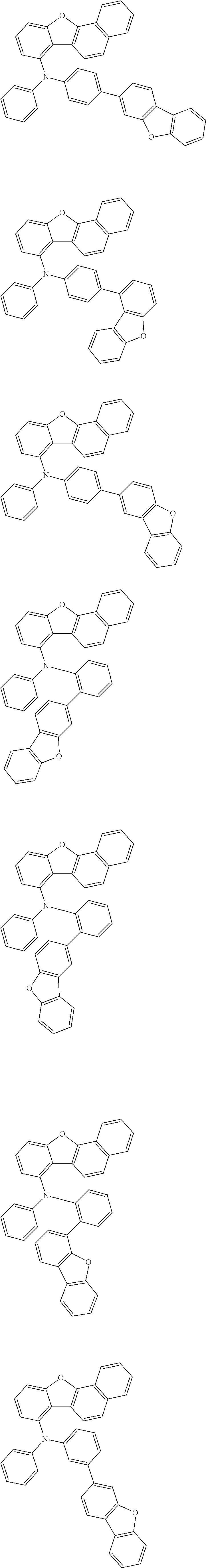

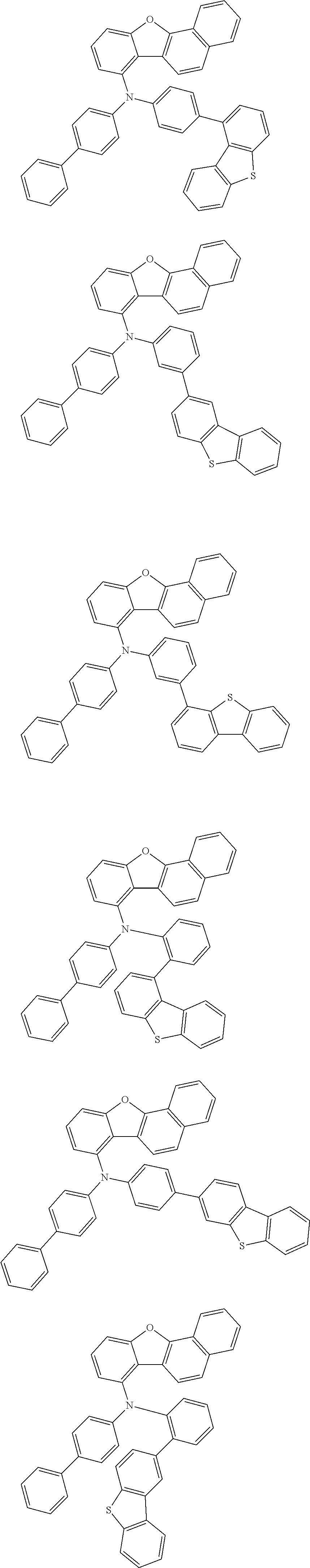

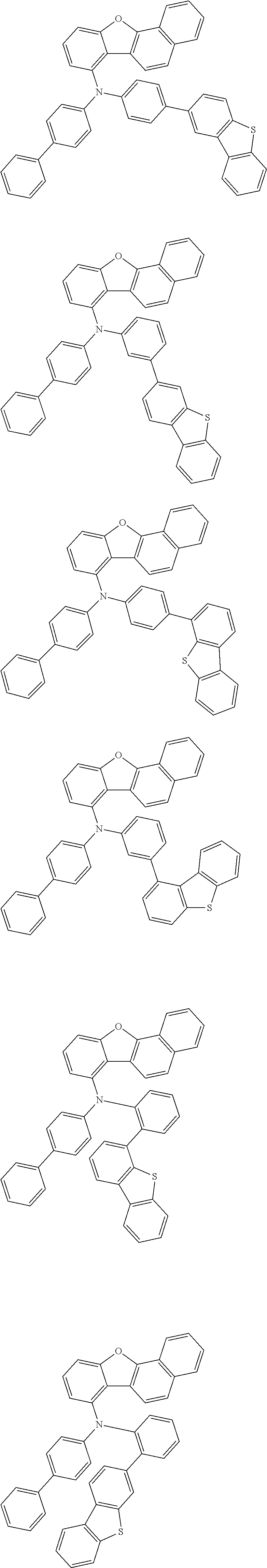

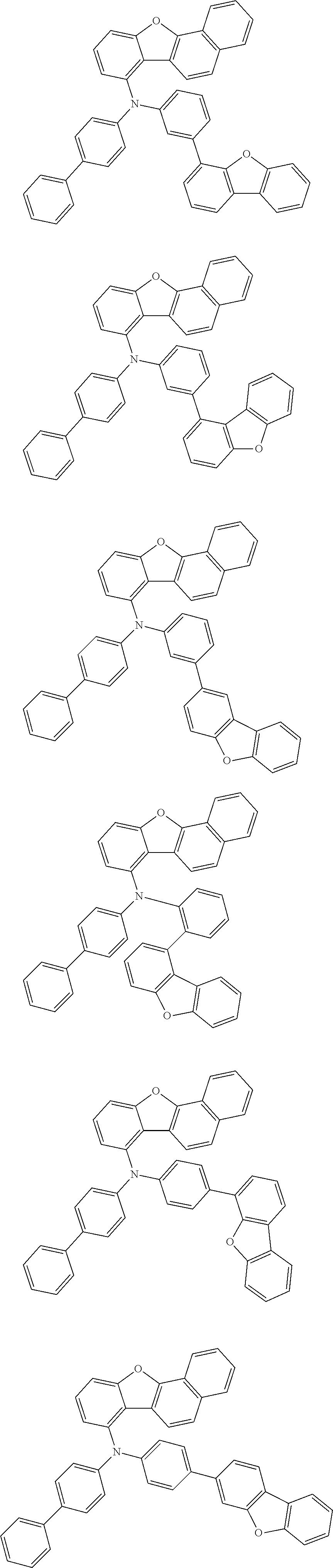

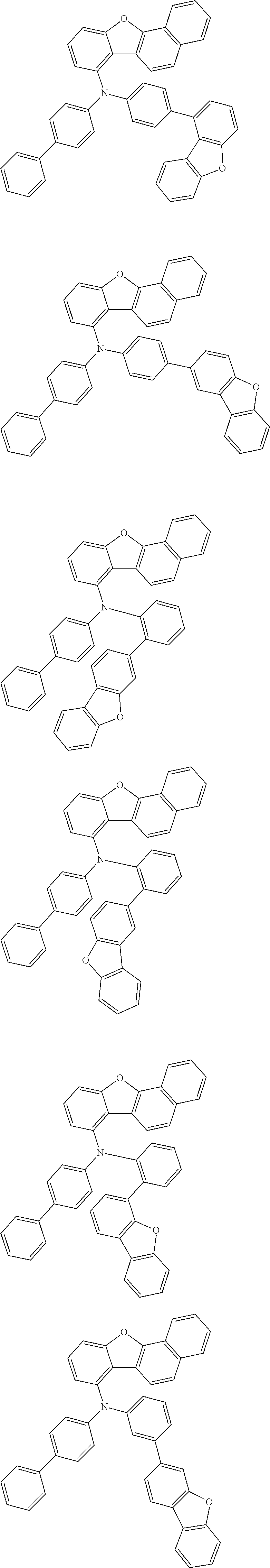

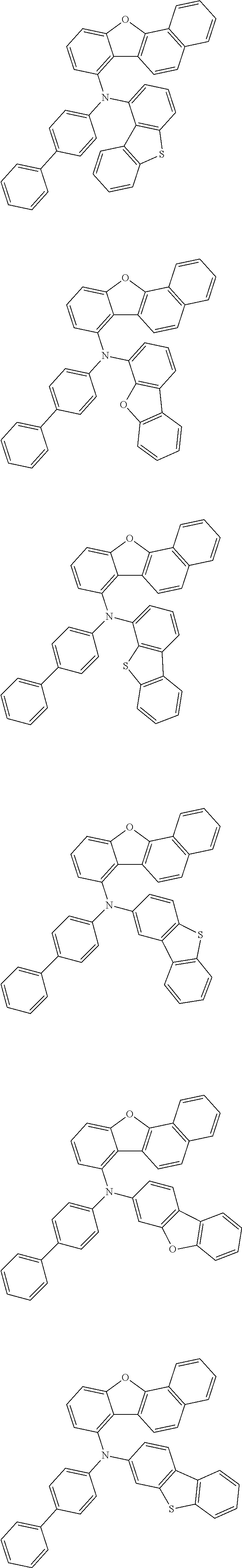 [SMILES: c1ccc(-c2ccc(N(c3cccc4oc5c6ccccc6ccc5c34)c3cccc4sc5ccccc5c34)cc2)cc1 ; c1ccc(-c2ccc(N(c3cccc4c3oc3ccccc34)c3cccc4oc5c6ccccc6ccc5c34)cc2)cc1 ; c1ccc(-c2ccc(N(c3cccc4c3sc3ccccc34)c3cccc4oc5c6ccccc6ccc5c34)cc2)cc1 ; c1ccc(-c2ccc(N(c3ccc4sc5ccccc5c4c3)c3cccc4oc5c6ccccc6ccc5c34)cc2)cc1 ; c1ccc(-c2ccc(N(c3ccc4c(c3)oc3ccccc34)c3cccc4oc5c6ccccc6ccc5c34)cc2)cc1 ; c1ccc(-c2ccc(N(c3ccc4c(c3)sc3ccccc34)c3cccc4oc5c6ccccc6ccc5c34)cc2)cc1]

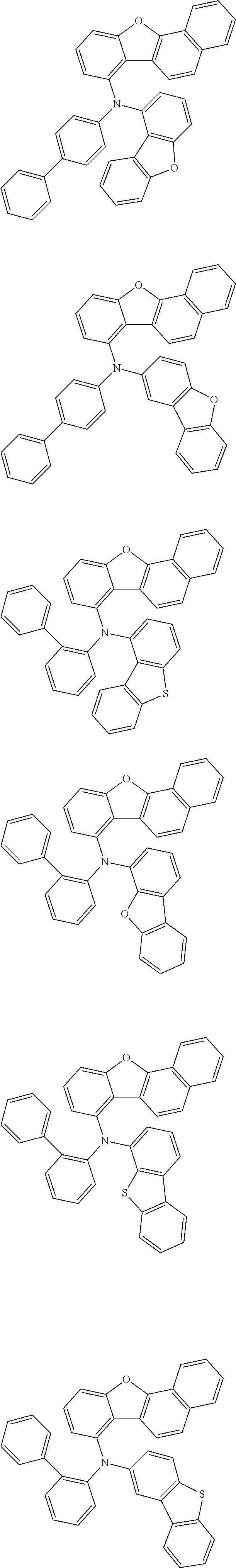 [SMILES: c1ccc(-c2ccc(N(c3cccc4oc5ccccc5c34)c3cccc4oc5c6ccccc6ccc5c34)cc2)cc1 ; c1ccc(-c2ccc(N(c3ccc4oc5ccccc5c4c3)c3cccc4oc5c6ccccc6ccc5c34)cc2)cc1 ; c1ccc(-c2ccccc2N(c2cccc3oc4c5ccccc5ccc4c23)c2cccc3sc4ccccc4c23)cc1 ; c1ccc(-c2ccccc2N(c2cccc3c2oc2ccccc23)c2cccc3oc4c5ccccc5ccc4c23)cc1 ; c1ccc(-c2ccccc2N(c2cccc3c2sc2ccccc23)c2cccc3oc4c5ccccc5ccc4c23)cc1 ; c1ccc(-c2ccccc2N(c2ccc3sc4ccccc4c3c2)c2cccc3oc4c5ccccc5ccc4c23)cc1]

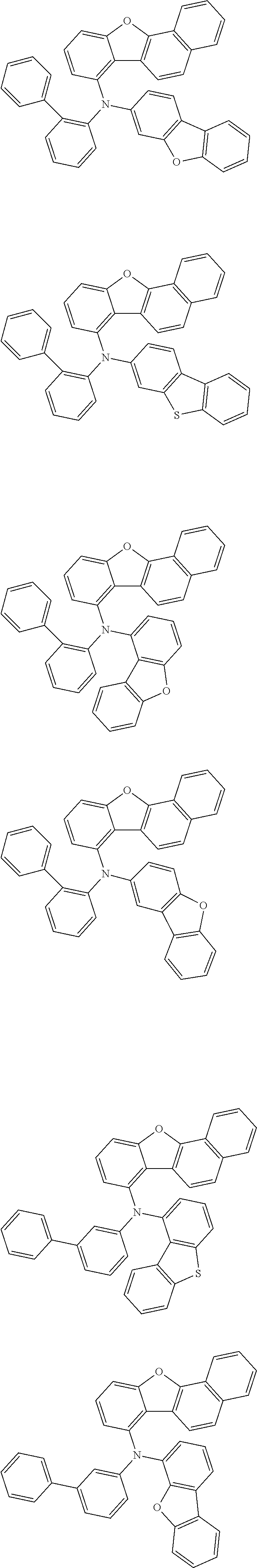 [SMILES: c1ccc(-c2ccccc2N(c2ccc3c(c2)oc2ccccc23)c2cccc3oc4c5ccccc5ccc4c23)cc1 ; c1ccc(-c2ccccc2N(c2ccc3c(c2)sc2ccccc23)c2cccc3oc4c5ccccc5ccc4c23)cc1 ; c1ccc(-c2ccccc2N(c2cccc3oc4ccccc4c23)c2cccc3oc4c5ccccc5ccc4c23)cc1 ; c1ccc(-c2ccccc2N(c2ccc3oc4ccccc4c3c2)c2cccc3oc4c5ccccc5ccc4c23)cc1 ; c1ccc(-c2cccc(N(c3cccc4oc5c6ccccc6ccc5c34)c3cccc4sc5ccccc5c34)c2)cc1 ; c1ccc(-c2cccc(N(c3cccc4c3oc3ccccc34)c3cccc4oc5c6ccccc6ccc5c34)c2)cc1]

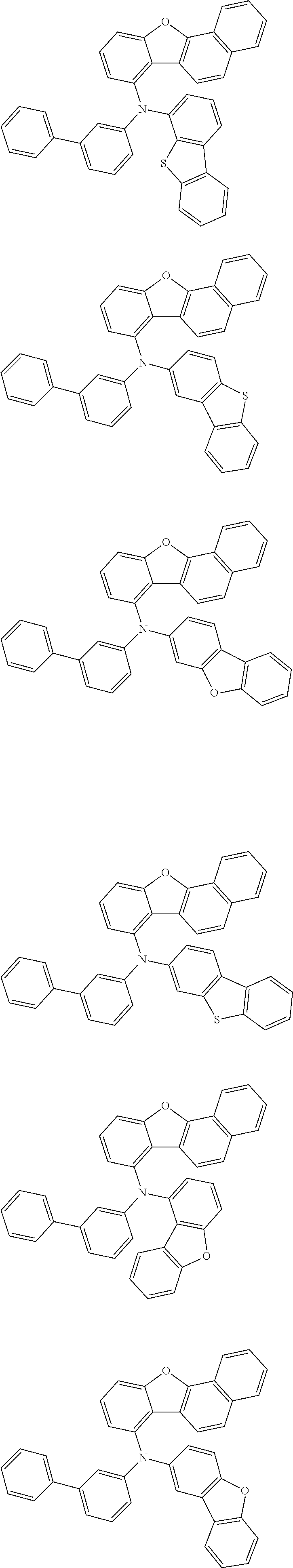 [SMILES: c1ccc(-c2cccc(N(c3cccc4c3sc3ccccc34)c3cccc4oc5c6ccccc6ccc5c34)c2)cc1 ; c1ccc(-c2cccc(N(c3ccc4sc5ccccc5c4c3)c3cccc4oc5c6ccccc6ccc5c34)c2)cc1 ; c1ccc(-c2cccc(N(c3ccc4c(c3)oc3ccccc34)c3cccc4oc5c6ccccc6ccc5c34)c2)cc1 ; c1ccc(-c2cccc(N(c3ccc4c(c3)sc3ccccc34)c3cccc4oc5c6ccccc6ccc5c34)c2)cc1 ; c1ccc(-c2cccc(N(c3cccc4oc5ccccc5c34)c3cccc4oc5c6ccccc6ccc5c34)c2)cc1 ; c1ccc(-c2cccc(N(c3ccc4oc5ccccc5c4c3)c3cccc4oc5c6ccccc6ccc5c34)c2)cc1]

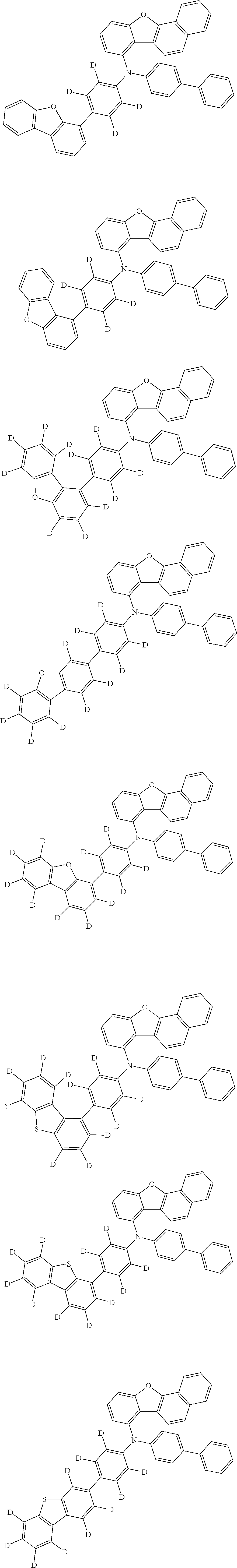

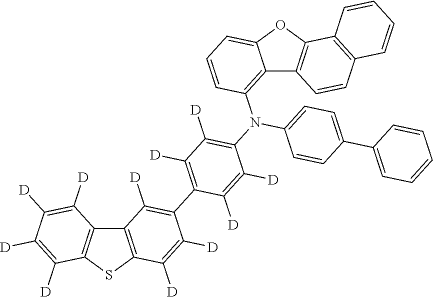 [SMILES: [2H]c1c([2H])c(N(c2ccc(-c3ccccc3)cc2)c2cccc3oc4c5ccccc5ccc4c23)c([2H])c([2H])c1-c1c([2H])c([2H])c2sc3c([2H])c([2H])c([2H])c([2H])c3c2c1[2H]]

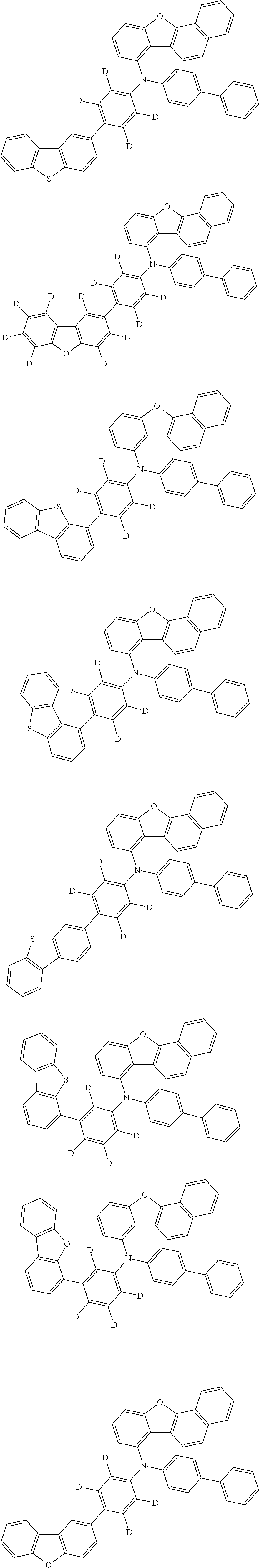

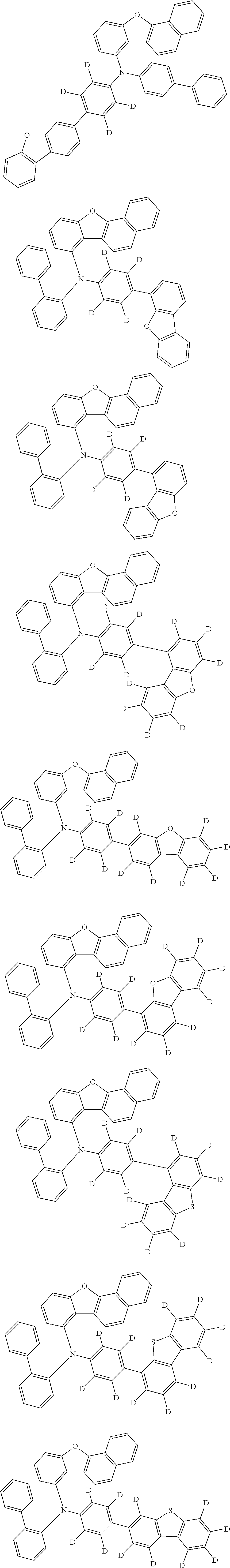

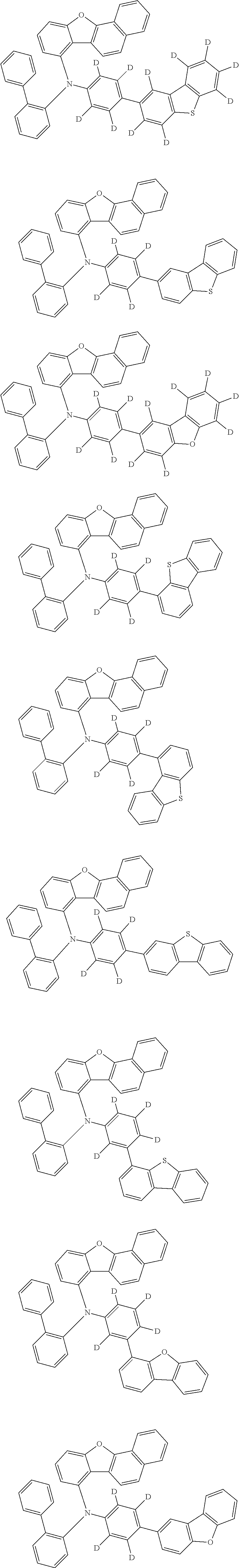

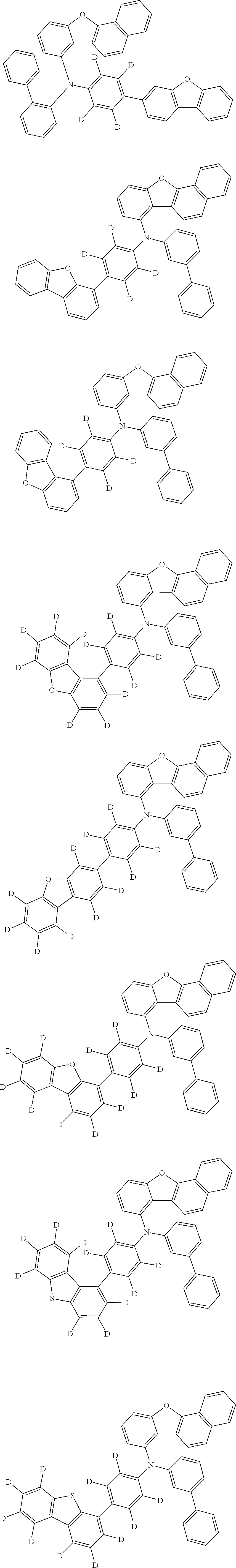

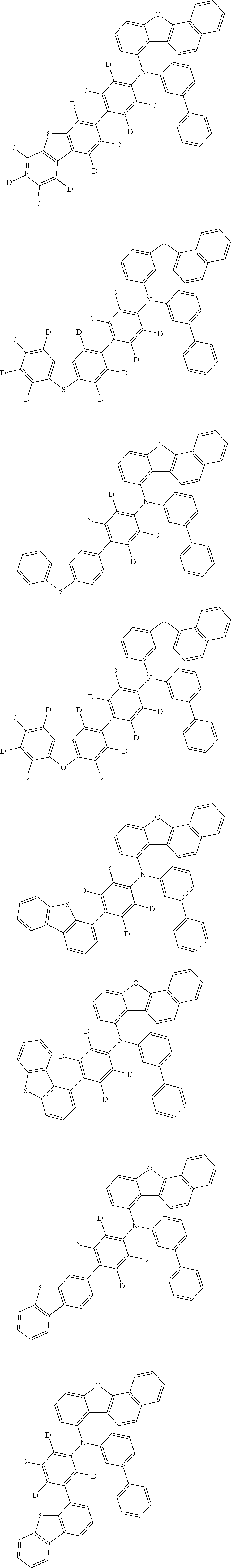

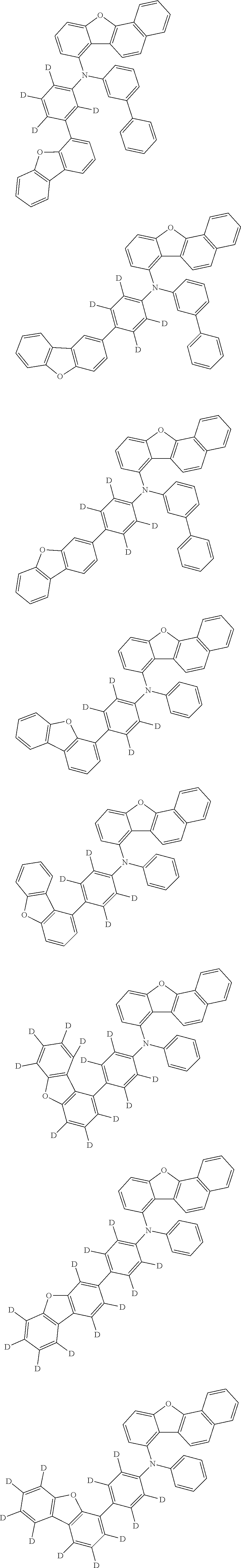

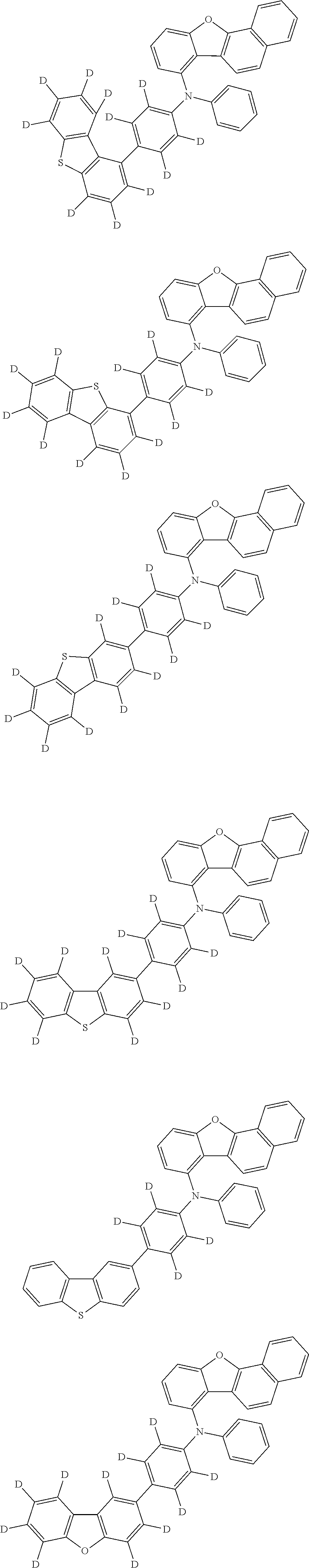

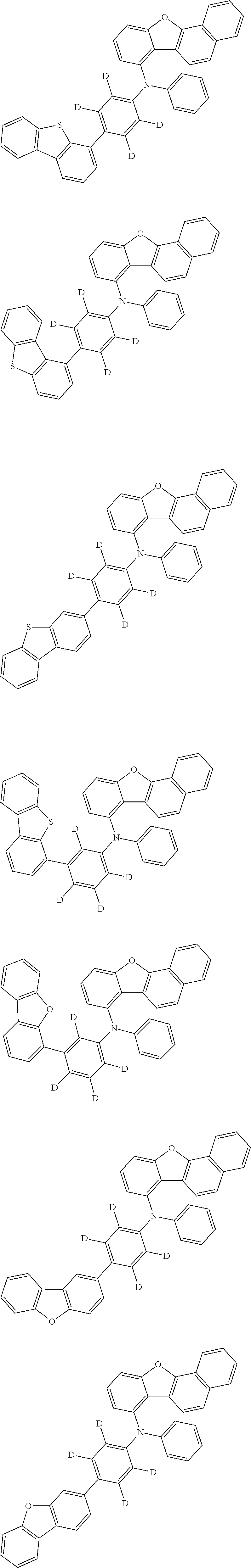

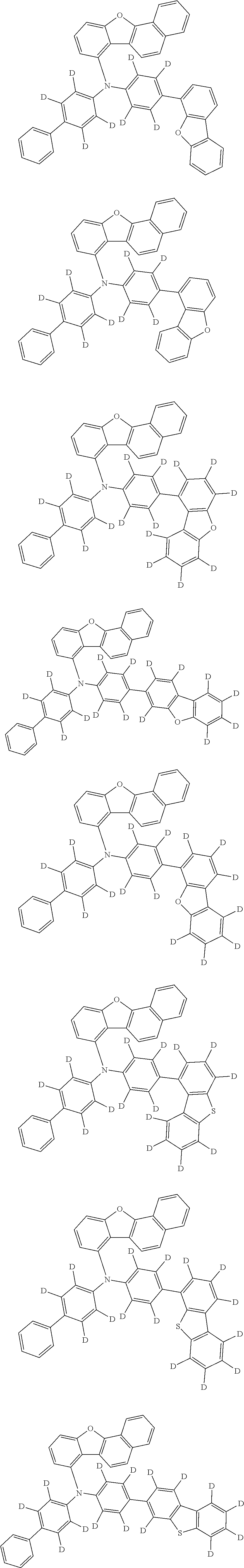

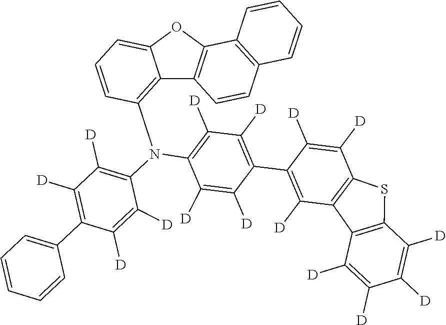 [SMILES: [2H]c1c([2H])c(N(c2c([2H])c([2H])c(-c3c([2H])c([2H])c4sc5c([2H])c([2H])c([2H])c([2H])c5c4c3[2H])c([2H])c2[2H])c2cccc3oc4c5ccccc5ccc4c23)c([2H])c([2H])c1-c1ccccc1]

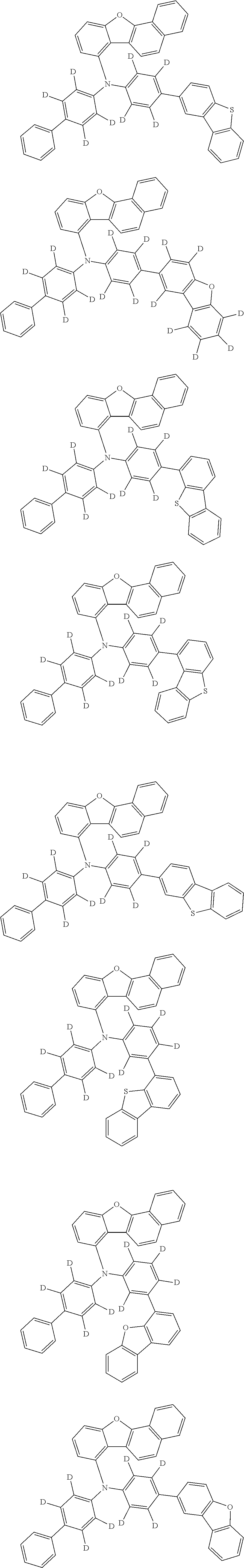

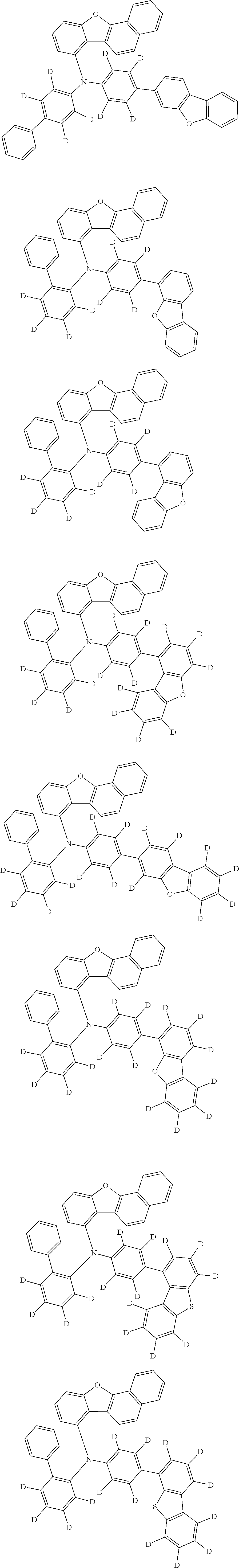

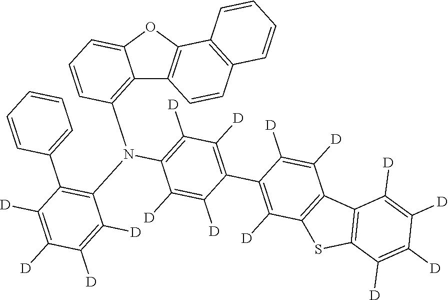 [SMILES: [2H]c1c([2H])c([2H])c(N(c2c([2H])c([2H])c(-c3c([2H])c([2H])c4c(sc5c([2H])c([2H])c([2H])c([2H])c54)c3[2H])c([2H])c2[2H])c2cccc3oc4c5ccccc5ccc4c23)c(-c2ccccc2)c1[2H]]

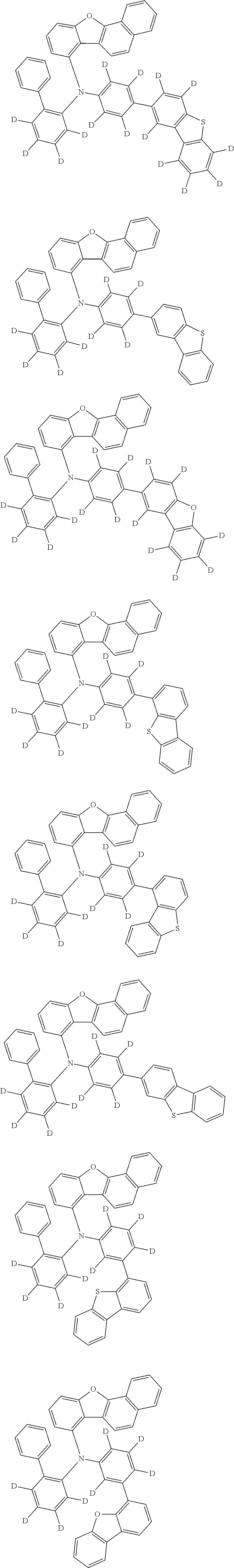

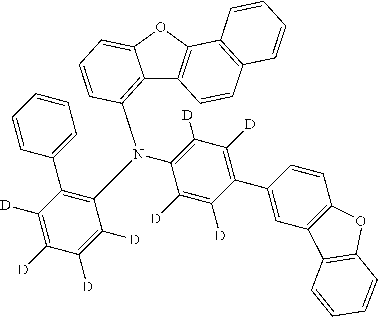 [SMILES: [2H]c1c([2H])c([2H])c(N(c2c([2H])c([2H])c(-c3ccc4oc5ccccc5c4c3)c([2H])c2[2H])c2cccc3oc4c5ccccc5ccc4c23)c(-c2ccccc2)c1[2H]]

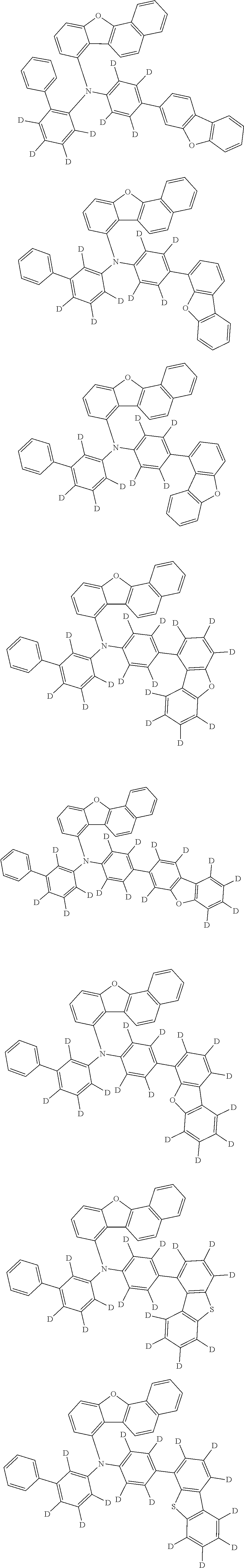

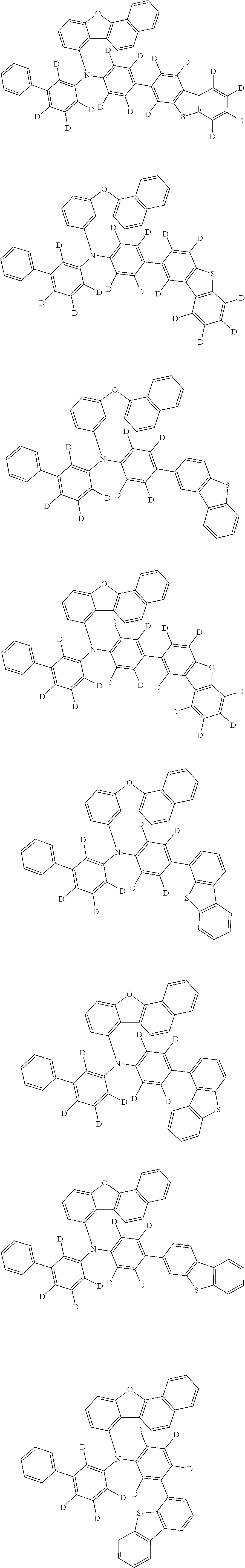

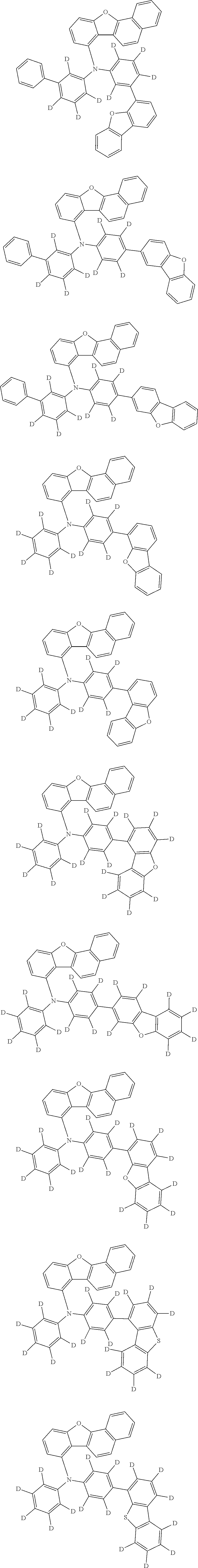

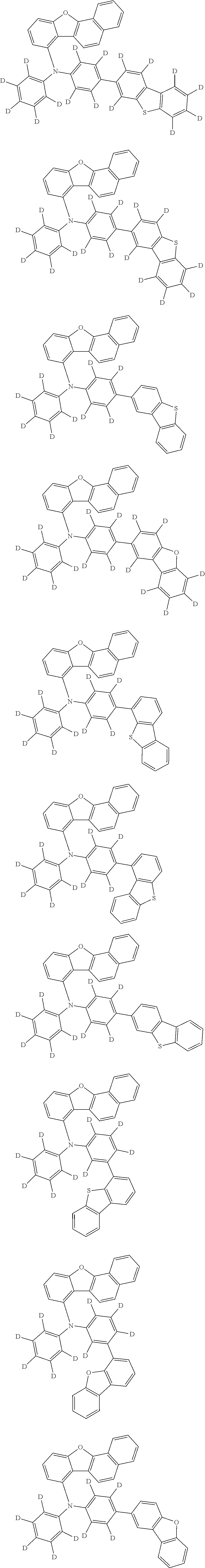

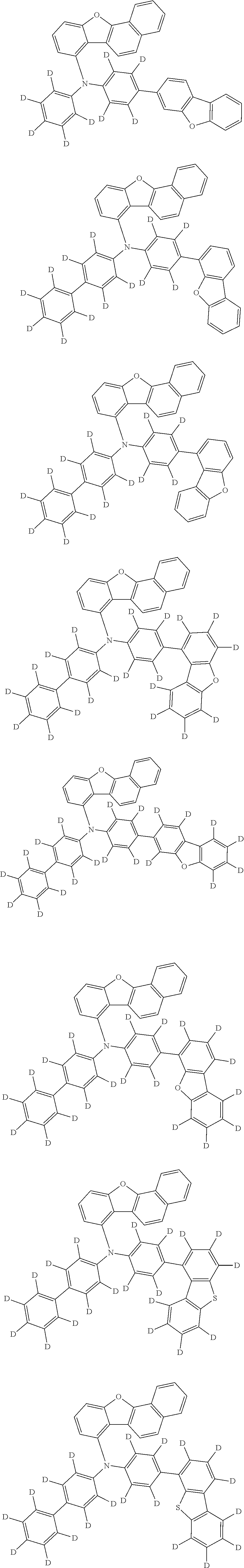

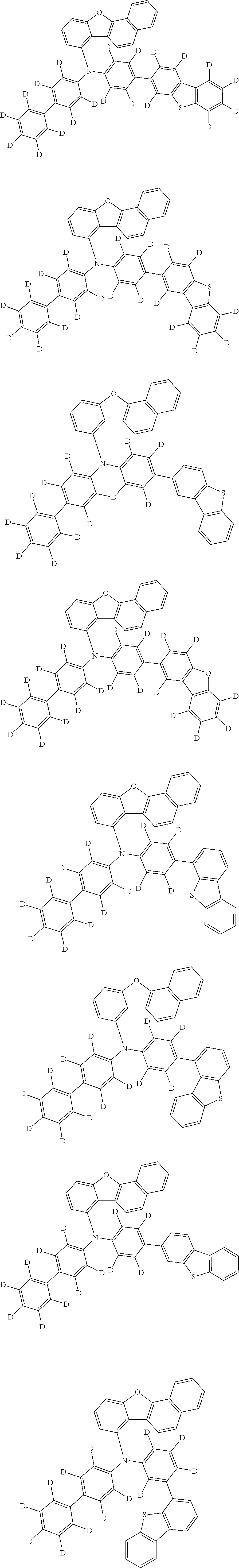

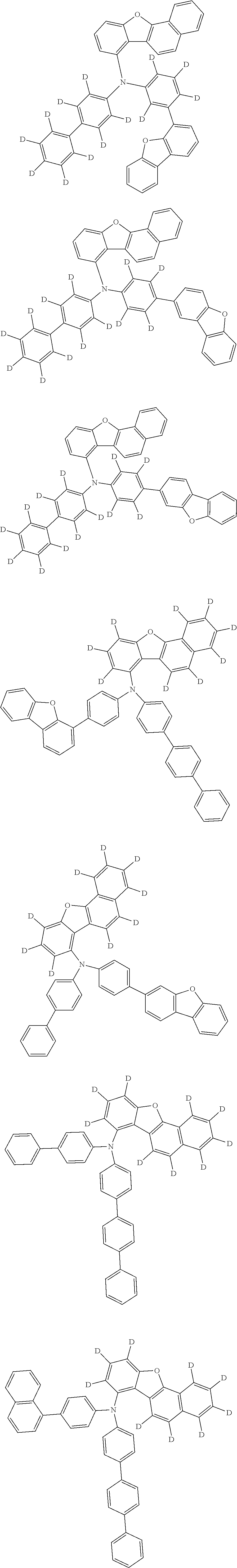

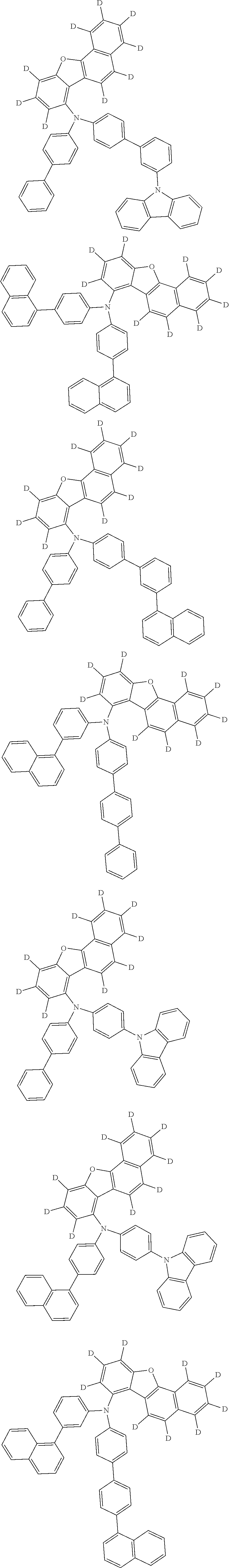

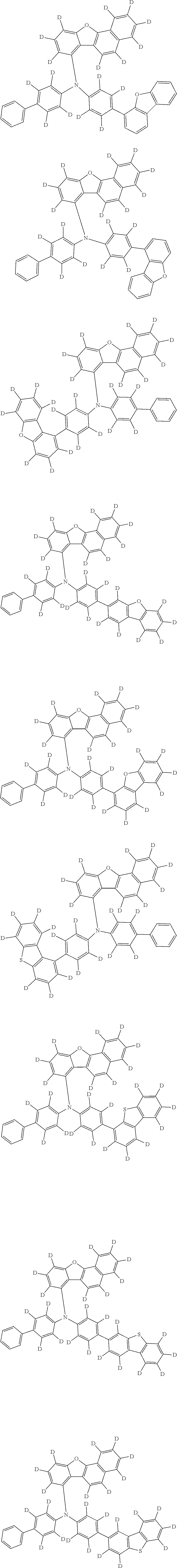

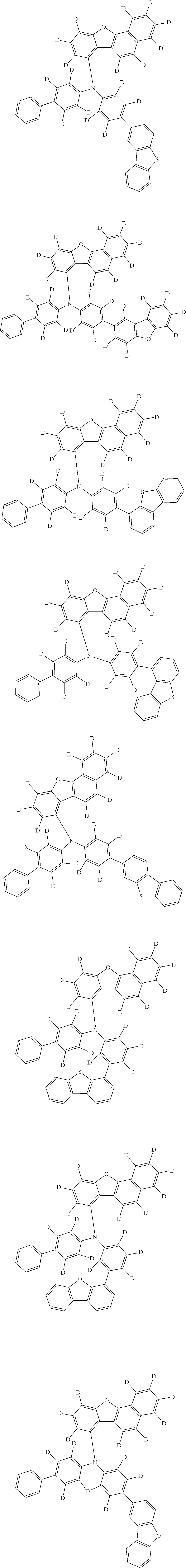

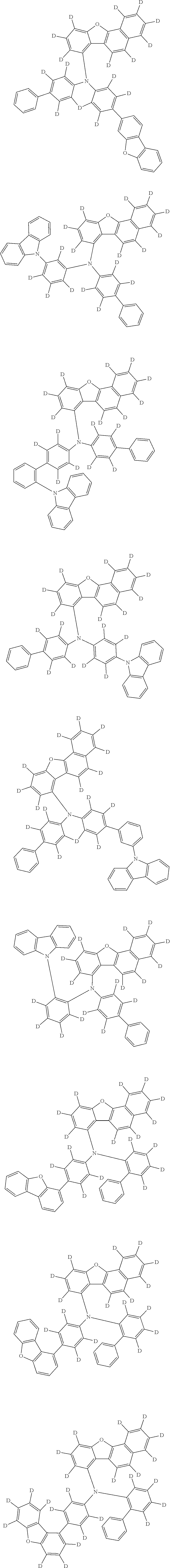

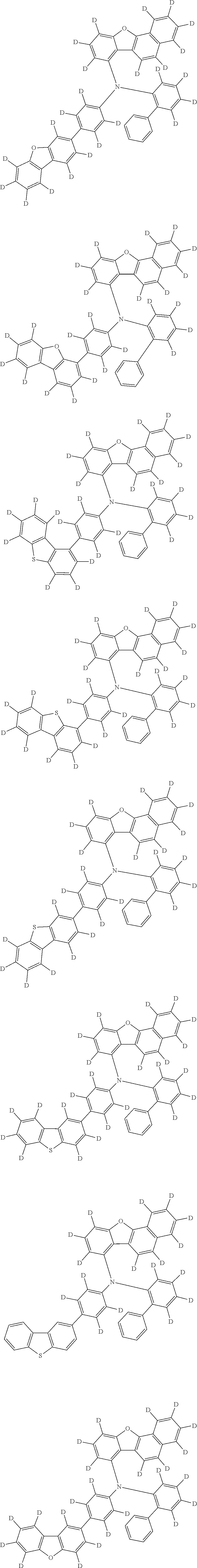

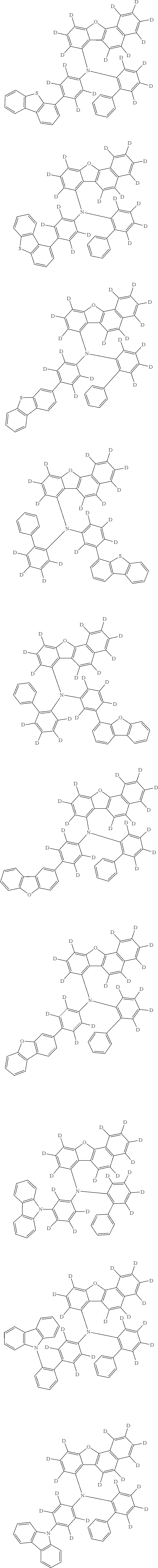

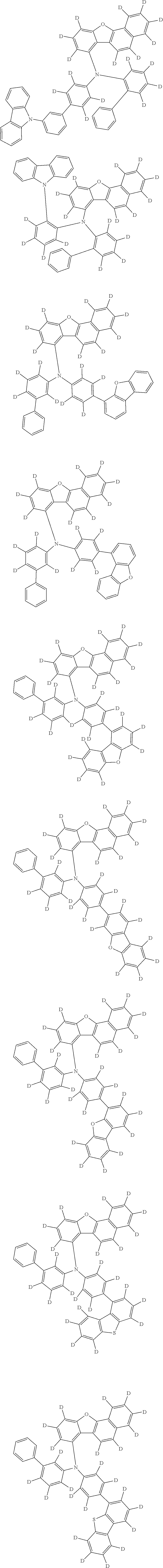

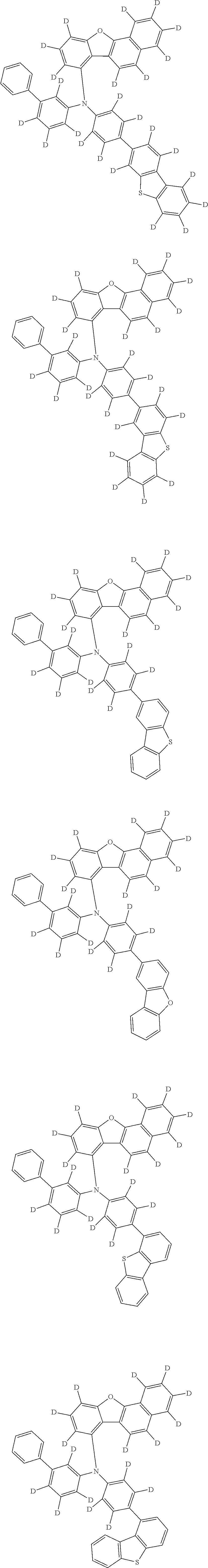

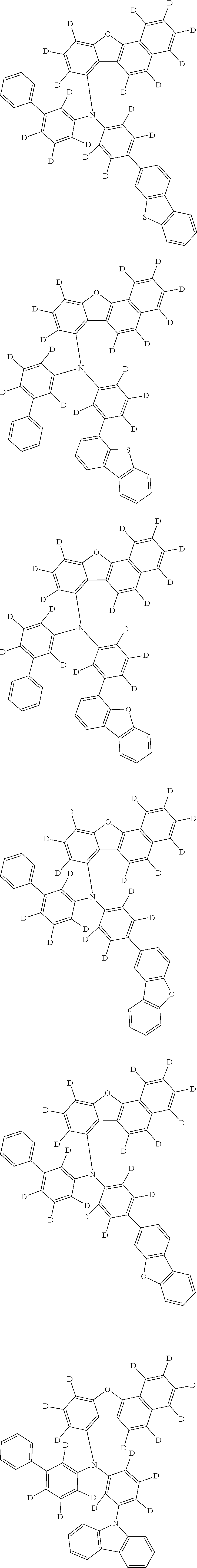

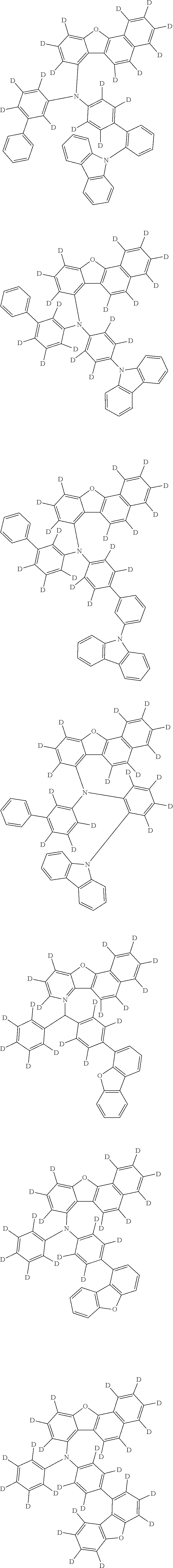

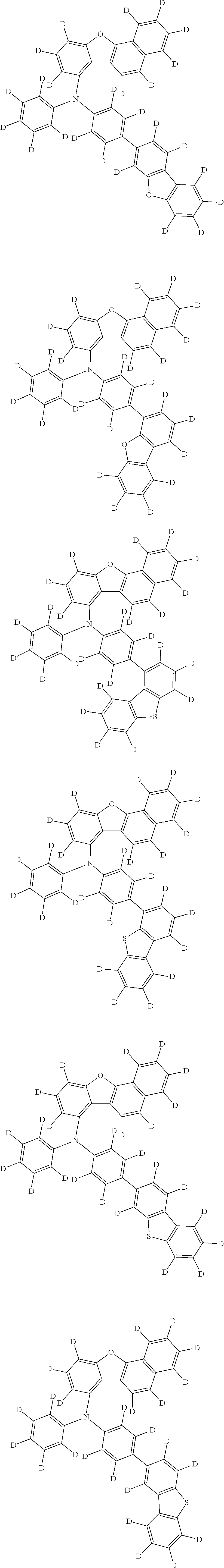

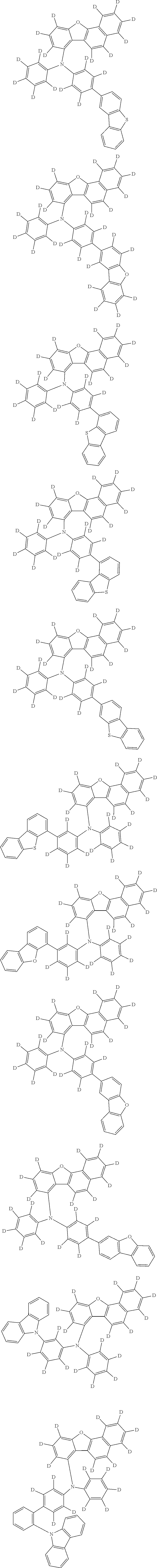

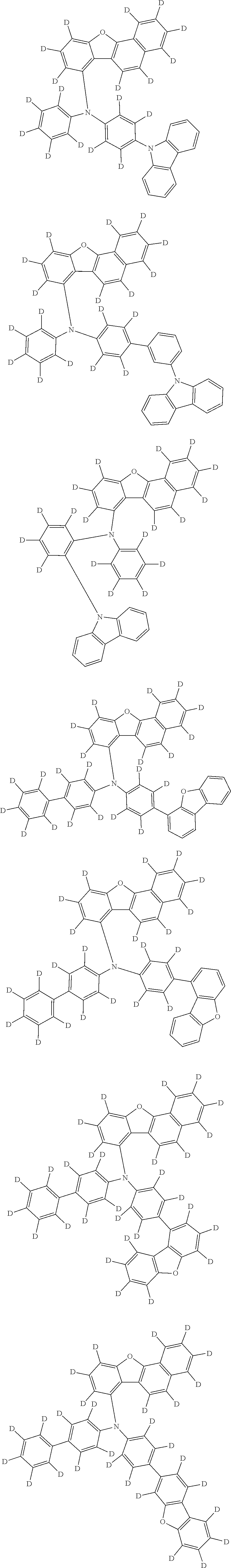

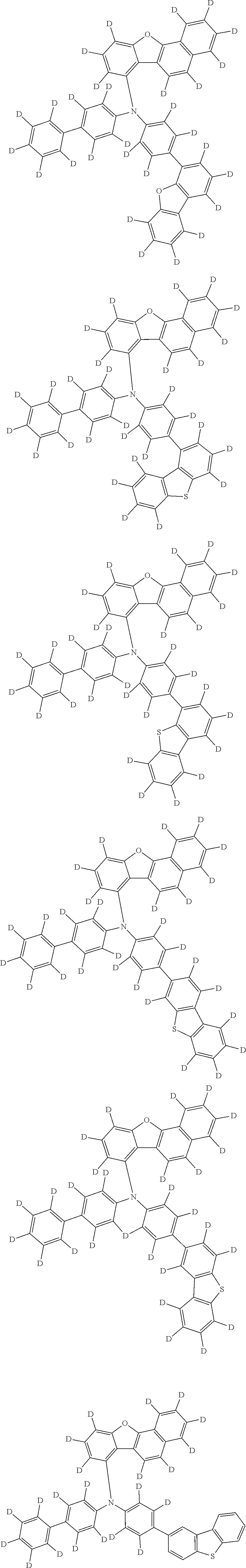

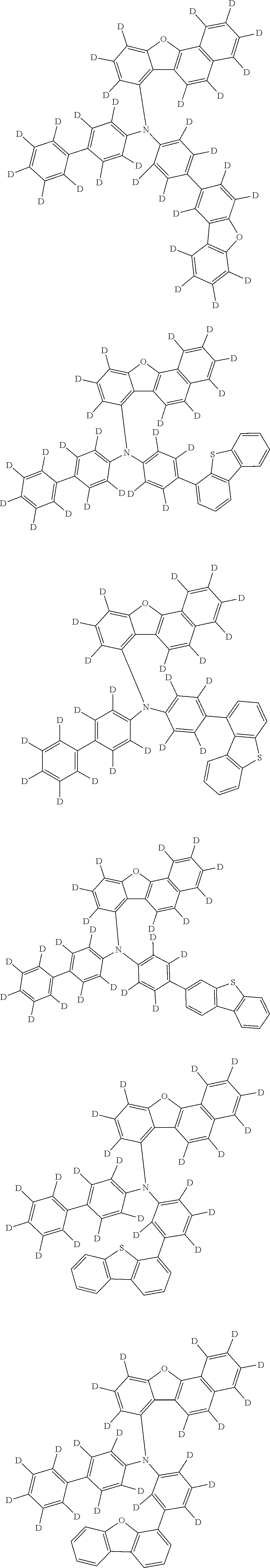

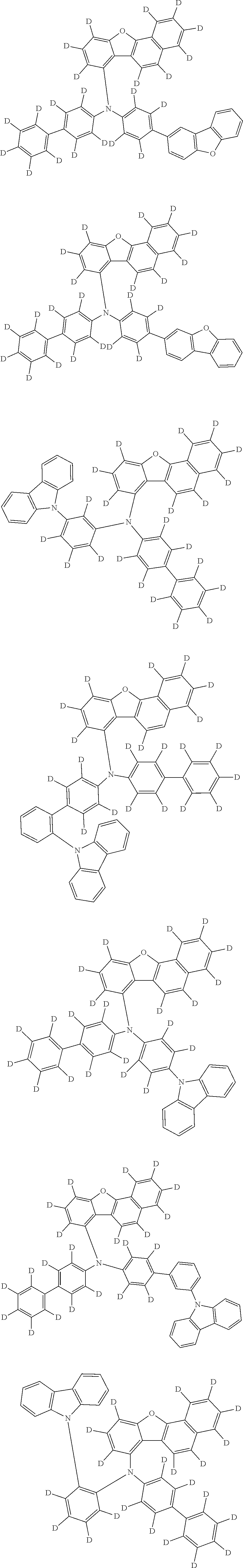

### Material for Organic EL Devices

The material for organic EL devices of one aspect of the present invention contains the compound (1). The content of the compound (1) in the material for organic EL devices can be 1% by mass or more (including 100%), and is preferably 10% by mass or more (including 100%), more preferably 50% by mass or more (including 100%), further preferably 80% by mass or more (including 100% especially preferably 90% by mass or more (including 100%). The material for organic EL devices of one aspect of the present invention is useful for the production of organic EL devices.

### Organic EL Device

The organic EL device of one embodiment of the present invention has a cathode, an anode, and a light emitting layer intervening between the cathode and the anode, and has an organic layer between the light emitting layer and the anode, in which the organic layer contains a compound represented by the following formula (2) (hereinafter this can be referred to as “compound (2)”).

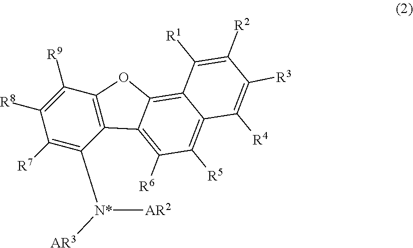

In the formula (2):

N* represents a central nitrogen atom.

R¹ to R⁹ each independently represent a hydrogen atom or the substituent A.

Ar² and Ar³ each independently represent a substituted or unsubstituted aryl group having 6 to 30 ring carbon atoms, or a substituted or unsubstituted heterocyclic group having 5 to 30 ring atoms.

The compound represented by the formula (2) is preferably represented by the following formula (3):

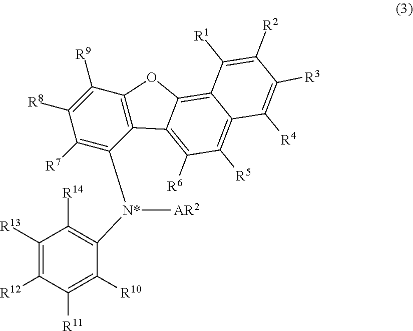

In the formula (3):

N*, R¹ to R⁹, and Ar² are as defined in the formula (2).

One of R¹⁰ to R¹⁴ is selected from a substituted or unsubstituted aryl group having 6 to 30 ring carbon atoms, and a substituted or unsubstituted heterocyclic group having 5 to 30 ring atoms. In the case where one of R¹⁰ to R¹⁴ is an aryl group, the aromatic ring contained in the aryl group is a 6-membered ring alone, and a fluorene group is excluded from the aryl group. The other R¹⁰ to R¹⁴ than the above one each independently represent a hydrogen atom or the substituent A.

Ar² is preferably represented by any of the formulae (1-a) to (1-f) that Ar¹ represents. In other words, the compound represented by the formula (2) is preferably represented by any of the formulae (1-1a) to (1-10, and is preferably represented by any of the formulae (1-1a-1) to (1-1a-5), the formulae (1-1b-1) to (1-1b-5), the formulae (1-1c-1) to (1-1c-5), the formulae (1-1d-1) to (1-1d-5), the formulae (1-1e-1) to (1-1e-3), and the formulae (1-1f-1) to (1-1f-5).

In one aspect, the compound represented by the formula (2) is represented by any of the formulae (1-1e-4) to (1-1e-6).

In the compound represented by the formula (2):
(2-1) all R¹ to R⁹ can be hydrogen atoms,
(2-2) the substituted or unsubstituted aryl group having 6 to 30 ring carbon atoms that Ar² represents can be unsubstituted,
(2-3) the substituted or unsubstituted heterocyclic group having 5 to 30 ring atoms that Ar² represents can be unsubstituted,
(2-4) the substituted or unsubstituted aryl group having 6 to 30 ring carbon atoms that Ar³ represents can be unsubstituted,
(2-5) the substituted or unsubstituted heterocyclic group having 5 to 30 ring atoms that Ar² represents can be unsubstituted.

In the compound represented by the formula (3):
(3-1) all R¹ to R⁹ can be hydrogen atoms,
(3-2) the substituted or unsubstituted aryl group having 6 to 30 ring carbon atoms that Ar² represents can be unsubstituted,
(3-3) the substituted or unsubstituted heterocyclic group having 5 to 30 ring atoms that Ar² represents can be unsubstituted,
(3-3) the substituted or unsubstituted aryl group having 6 to 30 ring carbon atoms that R¹⁰ to R¹⁴ represent can be unsubstituted,
(3-4) the substituted or unsubstituted heterocyclic group having 5 to 30 ring atoms that R¹⁰ to R¹⁴ represent can be unsubstituted.

In the case where the compound (2) is represented by any of the formulae (1-1a) to (1-1f), the formulae (1-1a-1) to (1-1a-5), the formulae (1-1b-1) to (1-1b-5), the formulae (1-1c-1) to (1-1c-5), the formulae (1-1d-1) to (1-1d-5), the formulae (1-1e-1) to (1-1e-6), or the formulae (1-1f-1) to (1-1f-5), preferably, each site can be a hydrogen atom like that described for the compound (1).

In one aspect of the present invention, the compound (2) contained in the organic layer contains at least one deuterium atom.

At least one hydrogen atom selected from the following hydrogen atoms can be a deuterium atom:
a hydrogen atom that any of R¹ to R⁹ represents; a hydrogen atom of the substituted or unsubstituted alkyl group, the substituted or unsubstituted alkenyl group, the substituted or unsubstituted alkynyl group, the substituted or unsubstituted cycloalkyl group, the substituted or unsubstituted haloalkyl group, the substituted or unsubstituted alkoxy group, the substituted or unsubstituted haloalkoxy group, the substituted or unsubstituted alkylthio group, the substituted or unsubstituted aryloxy group, the substituted or unsubstituted arylthio group, the substituted or unsubstituted aralkyl group, or the mono, di or tri-substituted silyl group that any of R¹ to R⁹ represents;
a hydrogen atom of the substituted or unsubstituted aryl group having 6 to 30 ring carbon atoms that Ar² represents; a hydrogen atom of the substituted or unsubstituted heterocyclic group having 5 to 30 ring atoms that Ar² represents;
a hydrogen atom of the substituted or unsubstituted aryl group having 6 to 30 ring carbon atoms that Ar³ represents; a hydrogen atom of the substituted or unsubstituted heterocyclic group having 5 to 30 ring atoms that Ar³ represents.

In the formula (3), at least one hydrogen atom selected from the following hydrogen atoms can be a deuterium atom:
a hydrogen atom that R¹⁰ to R¹⁴ represent; a hydrogen atom of the substituted or unsubstituted aryl group having 6 to 30 ring carbon atoms that R¹⁰ to R¹⁴ represent; a hydrogen atom of the substituted or unsubstituted heterocyclic group having 5 to 30 ring atoms that R¹⁰ to R¹⁴ represent.

Examples of the organic layer that contains the compound (2) include a hole transporting zone (such as a hole injecting layer, a hole transporting layer, an electron blocking layer, and an exciton blocking layer) intervening between the anode and the light emitting layer. The compound (2) is preferably used as a material for the hole transporting zone of a fluorescent EL device, more preferably a material for a hole transporting zone, even more preferably a material for a hole injecting layer, a hole transporting layer, an electron blocking layer or an exciton blocking layer, especially preferably a material for a hole injecting layer or a hole transporting layer.

The organic EL device of one aspect of the present invention can be a fluorescent light emission-type monochromatic light emitting device or a fluorescent/phosphorescent hybrid-type white light emitting device, and can be a simple type having a single light emitting unit or a tandem type having a plurality of light emitting units. The “light emitting unit” referred to herein refers to a minimum unit that emits light through recombination of injected holes and electrons, which includes organic layers among which at least one layer is a light emitting layer.

For example, as a representative device configuration of the simple type organic EL device, the following device configuration can be exemplified.

### (1) Anode/Light Emitting Unit/Cathode

The light emitting unit can be a multilayer type having a plurality of phosphorescent light emitting layers or fluorescent light emitting layers. In this case, a space layer may intervene between the light emitting layers for the purpose of preventing excitons generated in the phosphorescent light emitting layer from diffusing into the fluorescent light emitting layer. Representative layer configurations of the simple type light emitting unit are described below. Layers in parentheses are optional.
(a) (hole injecting layer/) hole transporting layer/fluorescent light emitting layer/electron transporting layer (/electron injecting layer)
(b) (hole injecting layer/) hole transporting layer/first fluorescent light emitting layer/second fluorescent light emitting layer/electron transporting layer (/electron injecting layer)
(c) (hole injecting layer/) hole transporting layer/phosphorescent light emitting layer/space layer/fluorescent light emitting layer/electron transporting layer (/electron injecting layer)
(d) (hole injecting layer/) hole transporting layer/first phosphorescent light emitting layer/space layer/second phosphorescent light emitting layer/space layer/fluorescent light emitting layer/electron transporting layer (/electron injecting layer)
(e) (hole injecting layer/) hole transporting layer/phosphorescent light emitting layer/space layer/first fluorescent light emitting layer/second fluorescent light emitting layer/electron transporting layer (/electron injecting layer)
(f) (hole injecting layer/) hole transporting layer/electron blocking layer/fluorescent light emitting layer/electron transporting layer/(electron injection layer)
(g) (hole injecting layer/) hole transporting layer/exciton blocking layer/fluorescent light emitting layer/electron transporting layer (/electron injecting layer)
(h) (hole injecting layer/) first hole transporting layer/second hole transporting layer/fluorescent light emitting layer/electron transporting layer (/electron injecting layer)
(i) (hole injecting layer/) first hole transporting layer/second hole transporting layer/fluorescent light emitting layer/first electron transporting layer/second electron transporting layer (/electron injecting layer)
(j) (hole injecting layer/) hole transporting layer/hole blocking layer/electron transporting layer (/electron injecting layer)
(k) (hole injecting layer/) hole transporting layer/fluorescent light emitting layer/exciton blocking layer/electron transporting layer (/electron injecting layer)

The phosphorescent and fluorescent light emitting layers may emit emission colors different from each other, respectively. Specifically, in the light emitting unit (f), a layer configuration, such as (hole injecting layer/) hole transporting layer/first phosphorescent light emitting layer (red light emission)/second phosphorescent light emitting layer (green light emission)/space layer/fluorescent light emitting layer (blue light emission)/electron transporting layer, can be exemplified.

An electron blocking layer can be properly provided between each light emitting layer and the hole transporting layer or the space layer. A hole blocking layer can be properly provided between each light emitting layer and the electron transporting layer. The employment of the electron blocking layer or the hole blocking layer allows to improve the emission efficiency by trapping electrons or holes within the light emitting layer and increasing the probability of charge recombination in the light emitting layer.

As a representative device configuration of the tandem type organic EL device, the following device configuration can be exemplified.

### (2) Anode/First Light Emitting Unit/Intermediate Layer/Second Light Emitting Unit/Cathode

For example, each of the first light emitting unit and the second light emitting unit can be independently selected from the above-described light emitting units.

The intermediate layer is also generally referred to as an intermediate electrode, an intermediate conductive layer, a charge generation layer, an electron withdrawing layer, a connecting layer, or an intermediate insulating layer, and a known material configuration can be used, in which electrons are supplied to the first light emitting unit, and holes are supplied to the second light emitting unit.

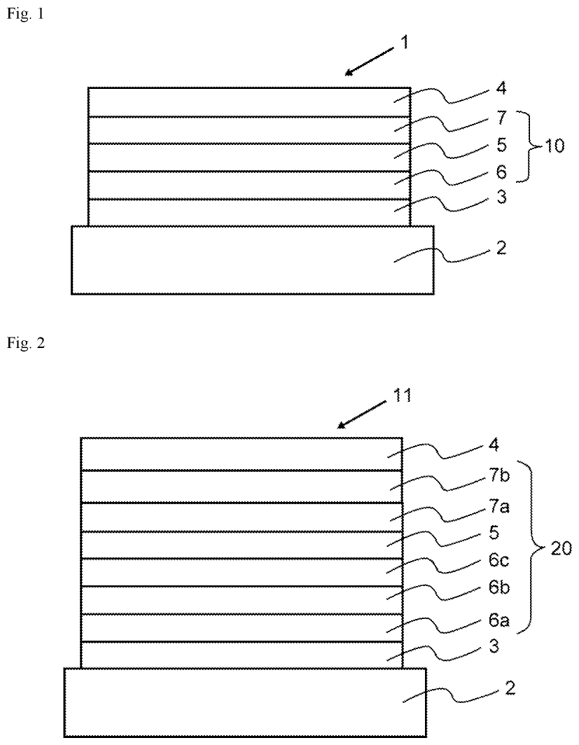

FIG. 1 is a schematic illustration showing an example of the configuration of the organic EL device of one aspect of the present invention. The organic EL device 1 of this example includes a substrate 2, an anode 3, a cathode 4, and a light emitting unit 10 disposed between the anode 3 and the cathode 4. The light emitting unit 10 includes a light emitting layer 5. A hole transporting zone 6 (such as a hole injecting layer and a hole transporting layer) is provided between the light emitting layer 5 and the anode 3, and an electron transporting zone 7 (such as an electron injecting layer and an electron transporting layer) is provided between the light emitting layer 5 and the cathode 4. In addition, an electron blocking layer (which is not shown in the figure) can be provided on the side of the anode 3 of the light emitting layer 5, and a hole blocking layer (which is not shown in the figure) can be provided on the side of the cathode 4 of the light emitting layer 5. According to the configuration, electrons and holes are trapped in the light emitting layer 5, thereby enabling one to further increase the production efficiency of excitons in the light emitting layer 5.

FIG. 2 is a schematic illustration showing another configuration of the organic EL device of one aspect of the present invention. An organic EL device 11 includes the substrate 2, the anode 3, the cathode 4, and a light emitting unit 20 disposed between the anode 3 and the cathode 4. The light emitting unit 20 includes the light emitting layer 5. A hole transporting zone disposed between the anode 3 and the light emitting layer 5 includes a hole injecting layer 6a, a first hole transporting layer 6b and a second hole transporting layer 6c. The electron transporting zone disposed between the light emitting layer 5 and the cathode 4 includes a first electron transporting layer 7a and a second electron transporting layer 7b..

In the present invention, a host combined with a fluorescent dopant (a fluorescent-emitting material) is referred to as a fluorescent host.

### Substrate

The substrate is used as a support of the organic EL device. Examples of the substrate include a plate of glass, quartz, and plastic. In addition, a flexible substrate can be used. Examples of the flexible substrate include a plastic substrate made of, polyimide, polycarbonate, polyarylate, polyether sulfone, polypropylene, polyester, polyvinyl fluoride, or polyvinyl chloride. In addition, an inorganic vapor deposition film can be used.

### Anode

It is preferred that a metal, an alloy, an electrically conductive compound, or a mixture thereof which has a high work function (specifically 4.0 eV or more) is used for the anode formed on the substrate. Specific examples thereof include indium oxide-tin oxide (ITO: Indium Tin Oxide), indium oxide-tin oxide containing silicon or silicon oxide, indium oxide-zinc oxide, indium oxide containing tungsten oxide and zinc oxide, and graphene. Besides, examples thereof include gold (Au), platinum (Pt), nickel (Ni), tungsten (W), chromium (Cr), molybdenum (Mo), iron (Fe), cobalt (Co), copper (Cu), palladium (Pd), titanium (Ti), or nitrides of the metals (for example, titanium nitride).

These materials are usually deposited by a sputtering method. For example, through a sputtering method, it is possible to form indium oxide-zinc oxide by using a target in which 1 to 10 wt % of zinc oxide is added to indium oxide, and to form indium oxide containing tungsten oxide and zinc oxide by using a target containing 0.5 to 5 wt % of tungsten oxide and 0.1 to 1 wt % of zinc oxide with respect to indium oxide. Besides, the manufacturing can be performed by a vacuum vapor deposition method, a coating method, an inkjet method, a spin coating method, or the like.

The hole injecting layer formed in contact with the anode is formed by using a material that facilitates hole injection regardless of a work function of the anode, and thus, it is possible to use materials generally used as an electrode material (for example, metals, alloys, electrically conductive compounds, or mixtures thereof, elements belonging to Group 1 or 2 of the periodic table of the elements).

It is also possible to use elements belonging to Group 1 or 2 of the periodic table of the elements, which are materials having low work functions, that is, alkali metals, such as lithium (Li) and cesium (Cs), alkaline earth metals, such as magnesium (Mg), calcium (Ca), and strontium (Sr), and alloys containing these (such as MgAg and AlLi), and rare earth metals, such as europium (Eu), and ytterbium (Yb) and alloys containing these. When the anode is formed by using the alkali metals, the alkaline earth metals, and alloys containing these, a vacuum vapor deposition method or a sputtering method can be used. Further, when a silver paste or the like is used, a coating method, an inkjet method, or the like can be used.

### Hole Injecting Layer

The hole injecting layer is a layer containing a material having a high hole injection capability (a hole injecting material) and is provided between the anode and the light emitting layer, or between the hole transporting layer, if existing, and the anode.

As the hole injecting material except the compound (2), molybdenum oxide, titanium oxide, vanadium oxide, rhenium oxide, ruthenium oxide, chromium oxide, zirconium oxide, hafnium oxide, tantalum oxide, silver oxide, tungsten oxide, and manganese oxide can be used.

Examples of the hole injecting layer material also include aromatic amine compounds as low-molecular weight organic compounds, such as 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), 4,4′-bis[N-(4-diphenylaminophenyl)-N-phenylamino]biphenyl (abbreviation: DPAB), 4,4′-bis(N-4-[N′-(3-methylphenyl)-N′-phenyl amino]phenyl-N-phenylamino)biphenyl (abbreviation: DNTPD), 1,3,5-tris[N-(4-diphenylaminophenyl)-N-phenylamino]benzene (abbreviation: DPA3B), 3-[N-(9-phenylcarbazole-3-yl)-N-phenylamino]-9-phenylcarbazole (abbreviation: PCzPCA1), 3,6-bis[N-(9-phenylcarbazole-3-yl)-N-phenylamino]-9-phenylcarbazole (abbreviation: PCzPCA2), and 3-[N-(1-naphthyl)-N-(9-phenylcarbazole-3-yl)amino]-9-phenylcarbazole (abbreviation: PCzPCN1).

High-molecular weight compounds (such as oligomers, dendrimers, and polymers) may also be used. Examples thereof include high-molecular weight compounds, such as poly(N-vinylcarbazole) (abbreviation: PVK), poly(4-vinyltriphenylamine) (abbreviation: PVTPA), poly[N-(4-N′-[4-(4-diphenylamino)phenyl]phenyl-N′-phenylaminophenyl)methacrylamide] (abbreviation: PTPDMA), and poly[N,N′-bis(4-butylphenyl)-N,N′-bis(phenyl)benzidine] (abbreviation: Poly-TPD). In addition, high-molecular weight compounds to which an acid is added, such as poly(3,4-ethylenedioxythiophene)/poly (styrene sulfonic acid) (PEDOT/PSS), and polyaniline/poly (styrenesulfonic acid) (PAni/PSS), can also be used.

Furthermore, it is also preferred to use an acceptor material, such as a hexaazatriphenylene (HAT) compound represented by formula (K).

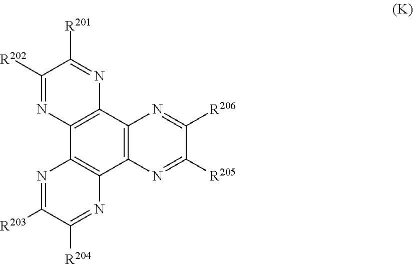

In the aforementioned formula, R²⁰¹ to R²⁰⁶ each independently represent a cyano group, —CONH₂, a carboxy group, or —COOR²⁰⁷ (R²⁰⁷ represents an alkyl group having 1 to 20 carbon atoms or a cycloalkyl group having 3 to 20 carbon atoms). In addition, adjacent two selected from R²⁰¹ and R²⁰², R²⁰³ and R²⁰⁴, and R²⁰⁵ and R²⁰⁶ can be bonded to each other to form a group represented by —CO—O—CO—.

Examples of R²⁰⁷ include a methyl group, an ethyl group, an n-propyl group, an isopropyl group, an n-butyl group, an isobutyl group, a t-butyl group, a cyclopentyl group, and a cyclohexyl group.

### Hole Transporting Layer

The hole transporting layer is a layer containing a material having a high hole transporting capability (a hole transporting material) and is provided between the anode and the light emitting layer, or between the hole injecting layer, if existing, and the light emitting layer. The compound (2) can be used as the hole transporting layer either singly or as combined with the compound mentioned below.

The hole transporting layer may have a single layer structure or a multilayer structure including two or more layers. For example, the hole transporting layer may have a two-layer structure including a first hole transporting layer (anode side) and a second hole transporting layer (cathode side). In one aspect of the present invention, the hole transporting layer having a single layer structure is preferably disposed adjacent to the light emitting layer, and the hole transporting layer that is closest to the cathode in the multilayer structure, such as the second hole transporting layer in the two-layer structure, is preferably disposed adjacent to the light emitting layer. In another aspect of the present invention, an electron blocking layer described later can be disposed between the hole transporting layer having a single layer structure and the light emitting layer, or between the hole transporting layer that is closest to the light emitting layer in the multilayer structure and the light emitting layer.

In the hole transporting layer of a two-layer structure, the compound (2) can be contained in the first hole transporting layer or the second hole transporting layer, or can be contained in both.

In one aspect of the present invention, the compound (2) is preferably contained in the first hole transporting layer alone, and in another aspect, the compound (2) is preferably contained in the second hole transporting layer alone, and in still another aspect, the compound (2) is preferably contained in the first hole transporting layer and the second hole transporting layer.

In one embodiment of the present invention, the compound (2) contained in one or both of the first hole transporting layer and the second hole transporting layer is preferably a protium compound from the viewpoint of production cost.

The protium compound is the compound (2) where all hydrogen atoms are protium atoms.

Accordingly, one aspect of the present invention preferably includes an organic EL device where one or both of the first hole transporting layer and the second hole transporting layer contain the compound (2) of substantially a protium compound alone. The “compound (2) of substantially a protium compound alone” means that the content ratio of a protium compound relative to the total amount of the compound (2) is 90 mol % or more, preferably 95 mol % or more, more preferably 99 mol % or more (each inclusive of 100%).

As the hole transporting material except the compound (2), for example, an aromatic amine compound, a carbazole derivative, and an anthracene derivative can be used.

Examples of the aromatic amine compound include 4,4′-bis[N-(1-naphthyl)-N-phenyl amino]biphenyl (abbreviation: NPB) or N,N′-bis(3-methylphenyl)-N,N′-diphenyl-[1,1′-biphenyl]-4,4′-diamine (abbreviation: TPD), 4-phenyl-4′-(9-phenylfluoren-9-yl)triphenylamine (abbreviation: BAFLP), 4,4′-bis[N-(9,9-dimethylfluoren-2-yl)-N-phenylamino]biphenyl (abbreviation: DFLDPBi), 4,4′,4″-tris(N,N-diphenylamino)triphenylamine (abbreviation: TDATA), 4,4′,4″-tris[N-(3-methylphenyl)-N-phenylamino]tri phenyl amine (abbreviation: MTDATA), and 4,4′-bis[N-(spiro-9,9′-bifluoren-2-yl)-N-phenylamino]biphenyl (abbreviation: BSPB). The aforementioned compounds have a hole mobility of 10⁻⁶ cm²/Vs or more.

Examples of the carbazole derivative include 4,4′-di(9-carbazolyl)biphenyl (abbreviation: CBP), 9-[4-(9-carbazolyl)phenyl]-10-phenylanthracene (abbreviation: CzPA), and 9-phenyl-3-[4-(10-phenyl-9-anthryl)phenyl]-9H-carbazole (abbreviation: PCzPA).

Examples of the anthracene derivative include 2-t-butyl-9,10-di(2-naphthyl)anthracene (abbreviation: t-BuDNA), 9,10-di(2-naphthyl)anthracene (abbreviation: DNA), and 9,10-diphenylanthracene (abbreviation: DPAnth).

High-molecular weight compounds, such as poly(N-vinylcarbazole) (abbreviation: PVK) and poly(4-vinyltriphenylamine) (abbreviation: PVTPA), can also be used.

However, compounds other than those as mentioned above can also be used so long as they are compounds high in the hole transporting capability rather than in the electron transporting capability.

### Dopant Material of Light Emitting Layer

The light emitting layer is a layer containing a material having a high light emitting property (a dopant material), and various materials can be used. For example, a fluorescent emitting material can be used as the dopant material. The fluorescent emitting material is a compound that emits light in a singlet excited state.

Examples of a blue-based fluorescent emitting material that can be used for the light emitting layer include a pyrene derivative, a styrylamine derivative, a chrysene derivative, a fluoranthene derivative, a fluorene derivative, a diamine derivative, and a triarylamine derivative. Specific examples thereof include N,N′-bis[4-(9H-carbazole-9-yl)phenyl]-N,N′-diphenylstilbene-4,4′-diamine (abbreviation: YGA2S), 4-(9H-carbazole-9-yl)-4′-(10-phenyl-9-anthryl)triphenylamine (abbreviation: YGAPA), and 4-(10-phenyl-9-anthryl)-4′-(9-phenyl-9H-carbazole-3-yl)triphenylamine (abbreviation: PCBAPA).

Examples of a green-based fluorescent emitting material that can be used for the light emitting layer include an aromatic amine derivative. Specific examples thereof include N-(9,10-diphenyl-2-anthryl)-N,9-diphenyl-9H-carbazole-3-amine (abbreviation: 2PCAPA), N-[9,10-bis(1,1′-biphenyl-2-yl)-2-anthryl]-N,9-diphenyl-9H-carbazole-3-amine (abbreviation: 2PCABPhA), N-(9,10-diphenyl-2-anthryl)-N,N′,N′-tiphenyl-1,4-phenylenediamine (abbreviation: 2DPAPA), N-[9,10-bis(1,1′-biphenyl-2-yl)-2-anthryl]-N,N′,N′-tiphenyl-1,4-phenylenediamine (abbreviation: 2DPABPhA), N-[9,10-bis(1,1′-biphenyl-2-yl)]-N-[4-(9H-carbazole-9-yl)phenyl]-N-phenylanthracene-2-amine (abbreviation: 2YGABPhA), and N,N,9-triphenylanthracene-9-amine (abbreviation: DPhAPhA).

Examples of a red-based fluorescent emitting material that can be used for the light emitting layer include a tetracene derivative and a diamine derivative. Specific examples thereof include N,N,N′,N′-tetrakis(4-methylphenyl)tetracene-5,11-diamine (abbreviation: p-mPhTD) and 7,14-diphenyl-N,N,N′,N′-tetrakis(4-methylphenyl)acenaphtho[1,2-a]fluoranthene-3,10-di amine (abbreviation: p-mPhAFD).

### Host Material of Light Emitting Layer

The light emitting layer may have a configuration in which the aforementioned dopant material is dispersed in another material (a host material). The host material is preferably a material that has a higher lowest unoccupied orbital level (LUMO level) and a lower highest occupied orbital level (HOMO level) than the dopant material.

Examples of the host material include:
(1) a metal complex, such as an aluminum complex, a beryllium complex, and a zinc complex,
(2) a heterocyclic compound, such as an oxadiazole derivative, a benzimidazole derivative, and a phenanthroline derivative,
(3) a fused aromatic compound, such as a carbazole derivative, an anthracene derivative, a phenanthrene derivative, a pyrene derivative, and a chrysene derivative, or
(4) an aromatic amine compound, such as a triarylamine derivative and a fused polycyclic aromatic amine derivative.

For example, the followings may be used:
metal complexes, such as tris(8-quinolinolato)aluminum(III) (abbreviation: Alq), tris(4-methyl-8-quinolinolato)aluminum(III) (abbreviation: Almq3), bis(10-hydroxybenzo[h]quinolinato)beryllium(II) (abbreviation: BeBg2), bis(2-methyl-8-quinolinolato)(4-phenylphenolato)aluminum(III) (abbreviation: BAIq), bis(8-quinolinolato)zinc(II) (abbreviation: Znq), bis[2-(2-benzoxazolyl)phenolato]zinc(II) (abbreviation: ZnPBO), and bis[2-(2-benzothiazolyl)phenolato]zinc(II) (abbreviation: ZnBTZ);
heterocyclic compounds, such as 2-(4-biphenylyl)-5-(4-tert-butylphenyl)-1,3,4-oxadiazole (abbreviation: PBD), 1,3-bis[5-(p-tert-butylphenyl)-1,3,4-oxadiazole-2-yl]benzene (abbreviation: OXD-7), 3-(4-biphenylyl)-4-phenyl-5-(4-tert-butylphenyl)-1,2,4-triazole (abbreviation: TAZ), 2,2′,2″-(1,3,5-benzenetriyl)tris(1-phenyl-1H-benzimidazole) (abbreviation: TPBI), and bathophenanthroline (abbreviation: BPhen), bathocuproine (abbreviation: BCP);
fused aromatic compounds, such as 9-[4-(10-phenyl-9-anthryl)phenyl]-9H-carbazole (abbreviation: CzPA), 3,6-diphenyl-9-[4-(10-phenyl-9-anthryl)phenyl]-9H-carbazole (abbreviation: DPCzPA), 9,10-bis(3,5-diphenylphenyl)anthracene (abbreviation: DPPA), 9,10-di(2-naphthyl)anthracene (abbreviation: DNA), 2-tert-butyl-9,10-di(2-naphthyl)anthracene (abbreviation: t-BuDNA), 9,9′-bianthryl (abbreviation: BANT), 9,9′-(stilbene-3,3′-diyl)diphenanthrene (abbreviation: DPNS), 9,9′-(stilbene-4,4′-diyl)diphenanthrene (abbreviation: DPNS2), 3,3′,3″-(benzene-1,3,5-triyl)tripyrene (abbreviation: TPB3), 9,10-diphenylanthracene (abbreviation: DPAnth), and 6,12-dimethoxy-5,11-diphenylchrysene; and
aromatic amine compounds, such as N,N-diphenyl-9-[4-(10-phenyl-9-anthryl)phenyl]-9H-carbazole-3-amine (abbreviation: CzA1PA), 4-(10-phenyl-9-anthryl)triphenylamine (abbreviation: DPhPA), N,9-diphenyl-N-[4-(10-phenyl-9-anthryl)phenyl]-9H-carbazole-3-amine (abbreviation: PCAPA), N,9-diphenyl-N-4-[4-(10-phenyl-9-anthryl)phenyl]phenyl-9H-carbazole-3-amine (abbreviation: PCAPBA), N-(9,10-diphenyl-2-anthryl)-N,9-diphenyl-9H-carbazole-3-amine (abbreviation: 2PCAPA), 4,4′-bis[N-(1-naphthyl)-N-phenylamino]biphenyl (abbreviation: NPB or a-NPD), N,N′-bis(3-methylphenyl)-N,N′-diphenyl-[1,1′-biphenyl]-4,4′-diamine (abbreviation: TPD), 4,4′-bis[N-(9,9-dimethylfluoren-2-yl)-N-phenylamino]biphenyl (abbreviation: DFLDPBi), and 4,4′-bis[N-(spiro-9,9′-bifluoren-2-yl)-N-phenylamino]biphenyl (abbreviation: BSPB). A plurality of host materials can be used.

In particular, in the case of a blue fluorescent device, it is preferred to use the following anthracene compounds as the host material.

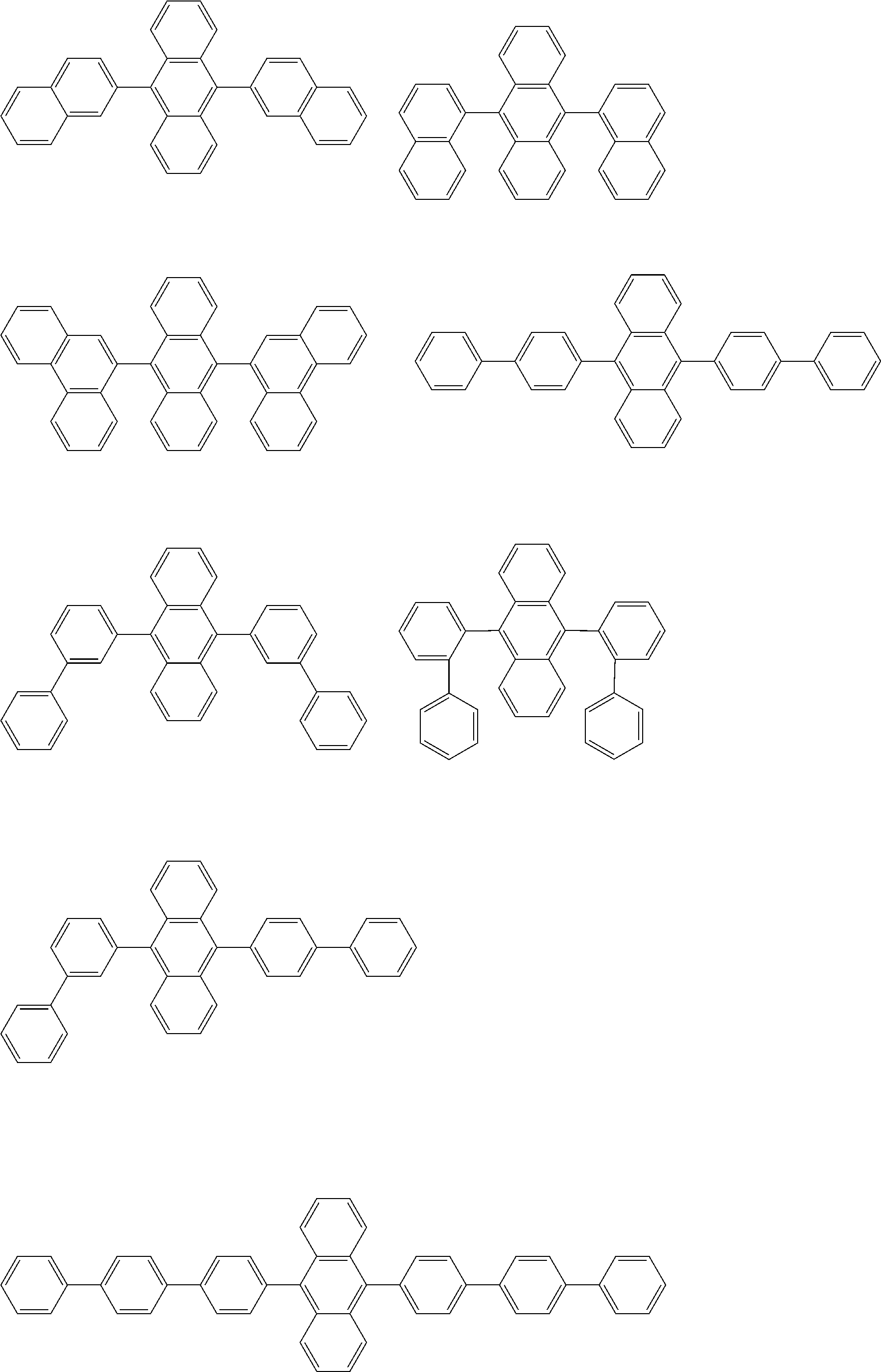

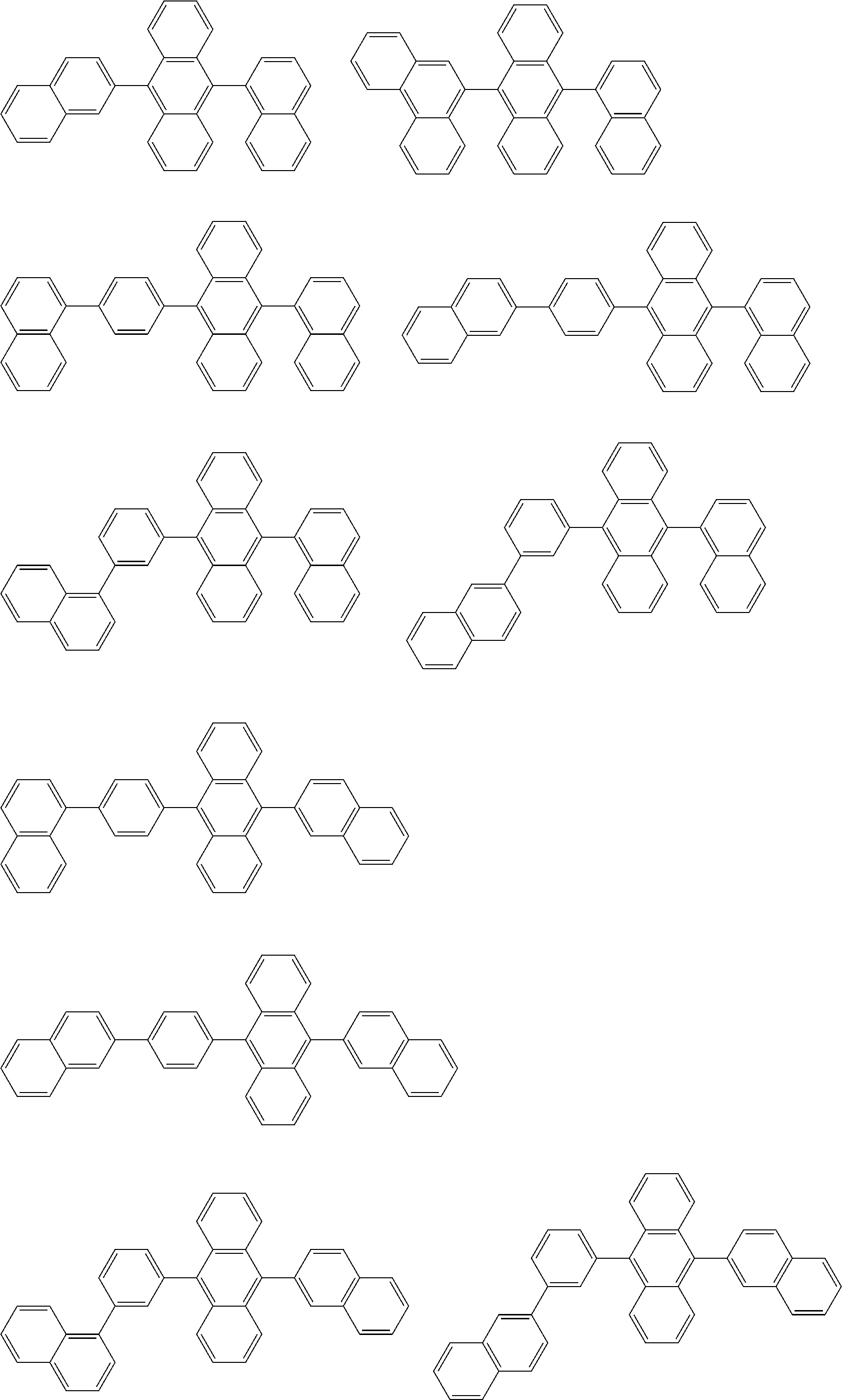

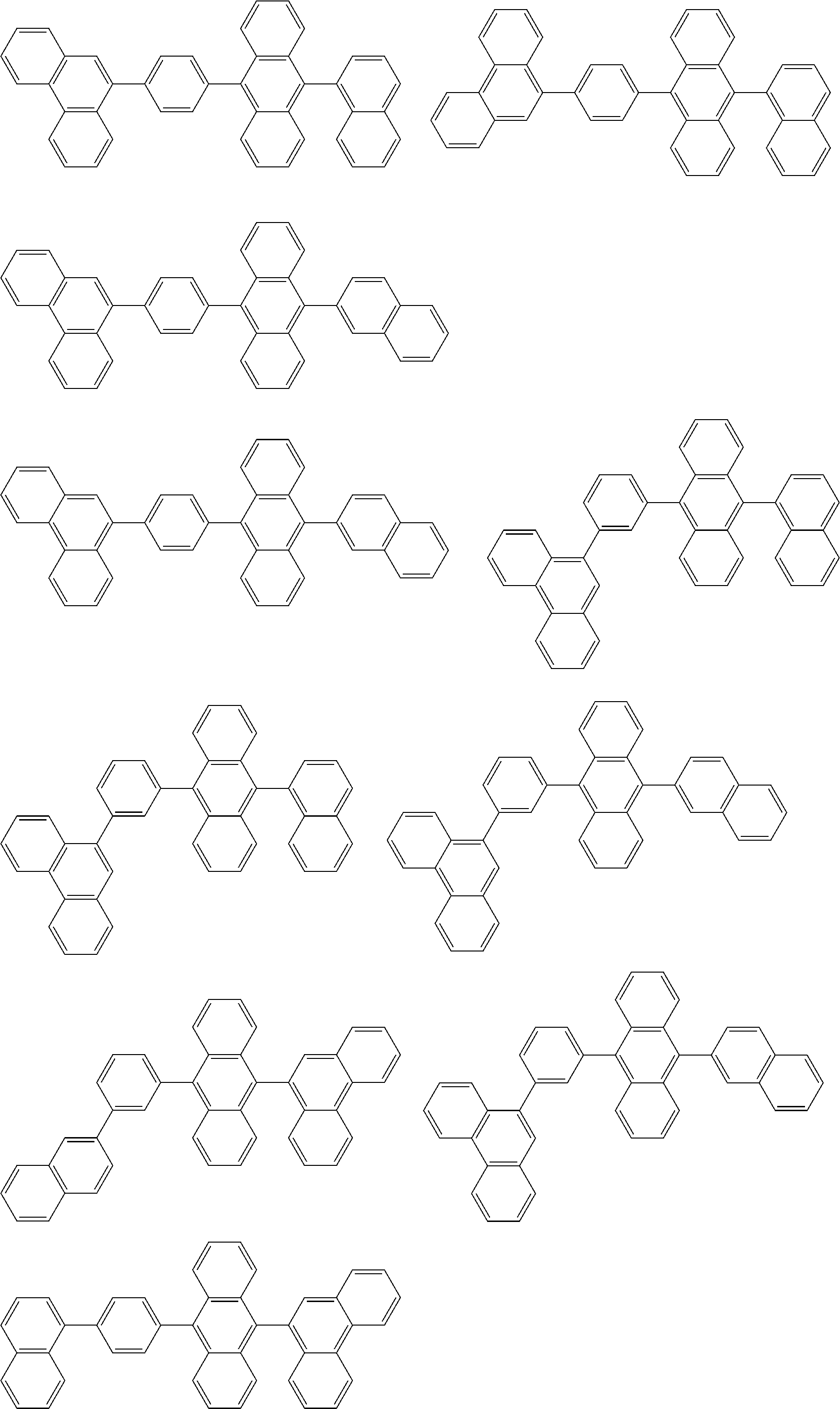

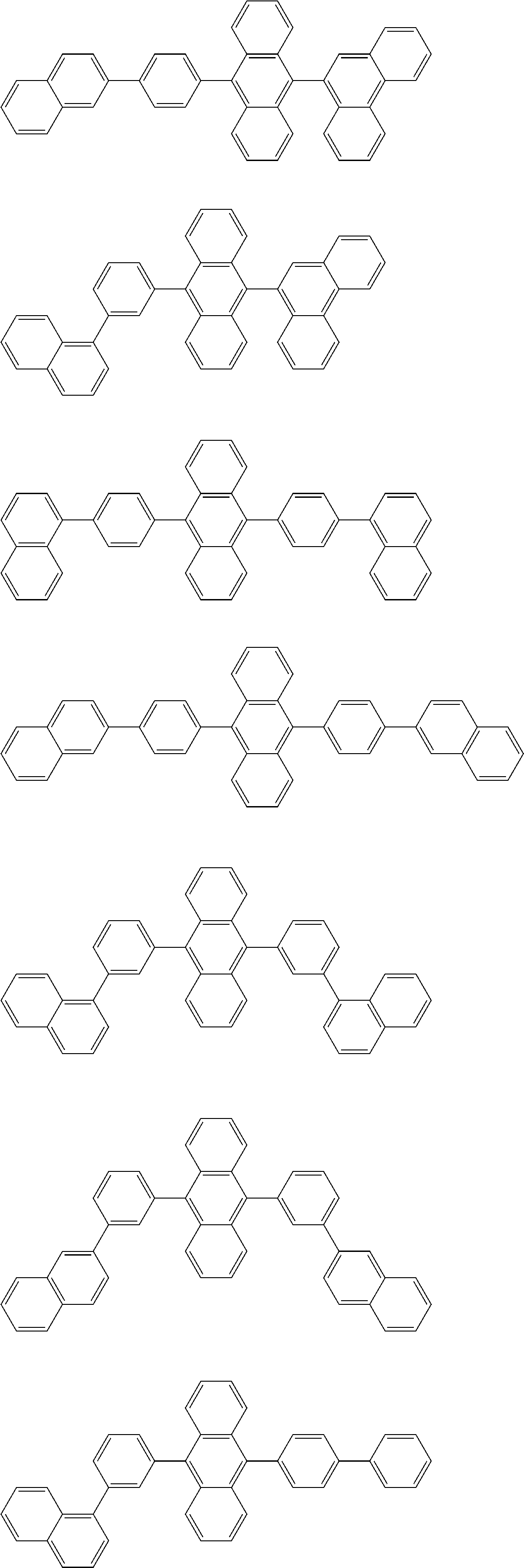

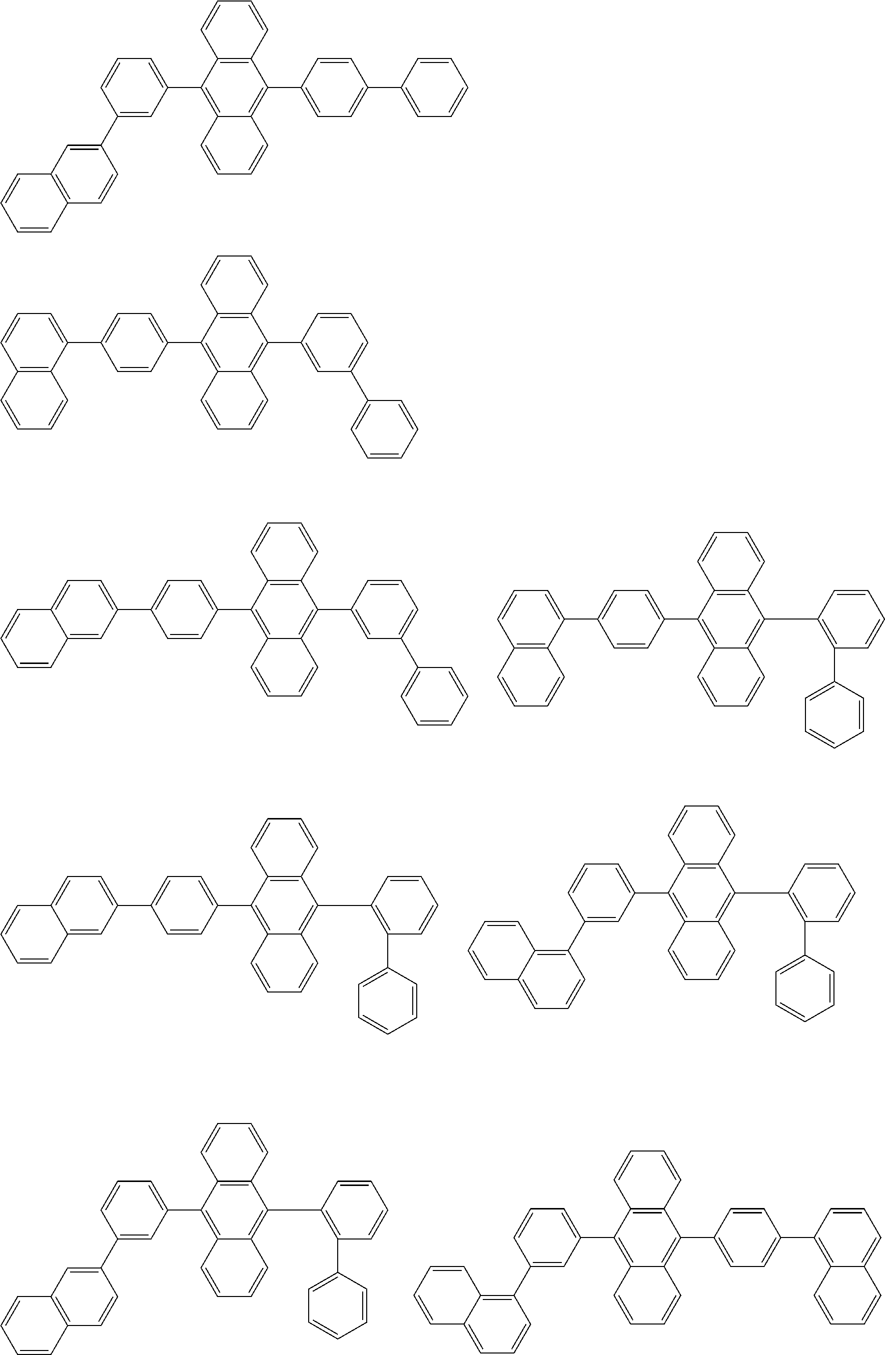

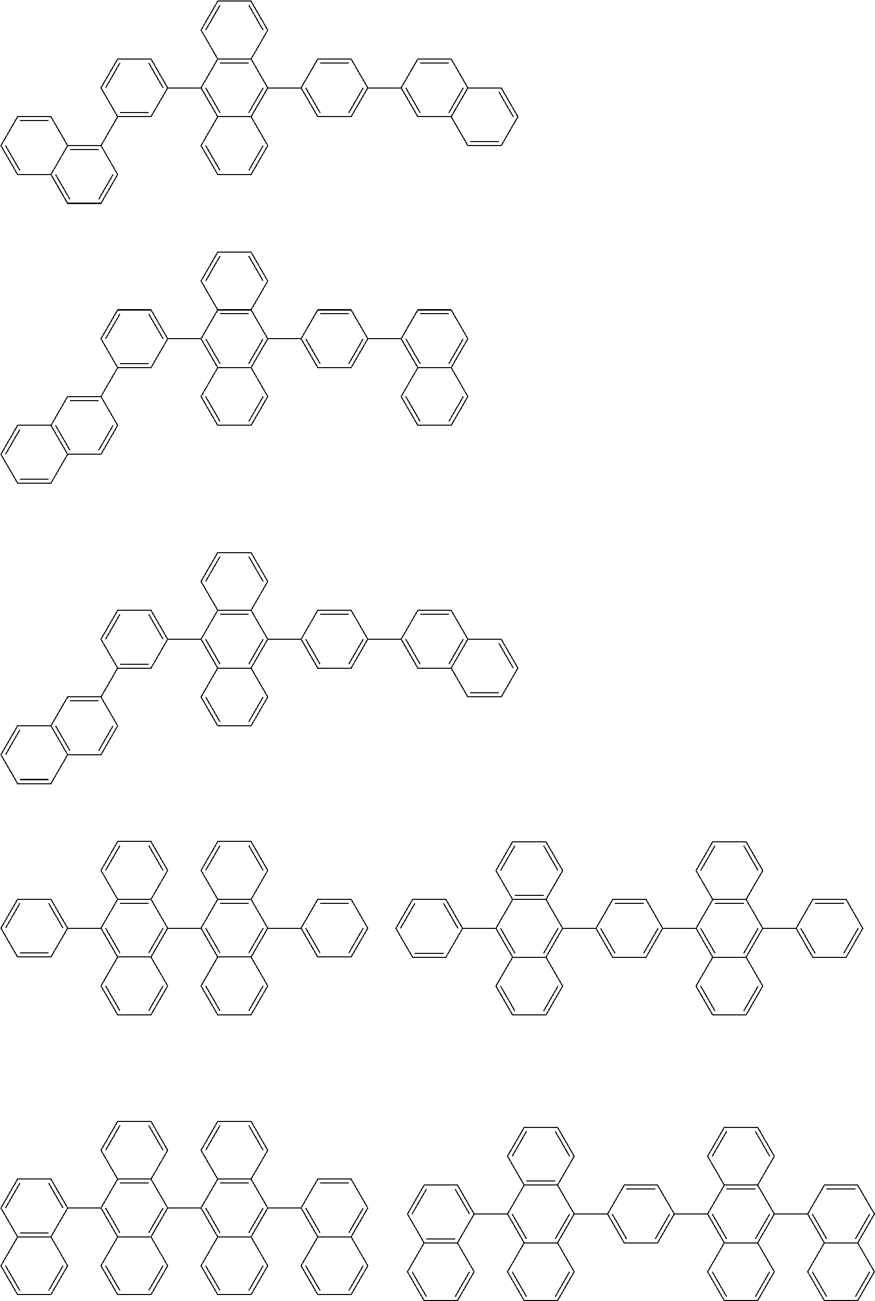

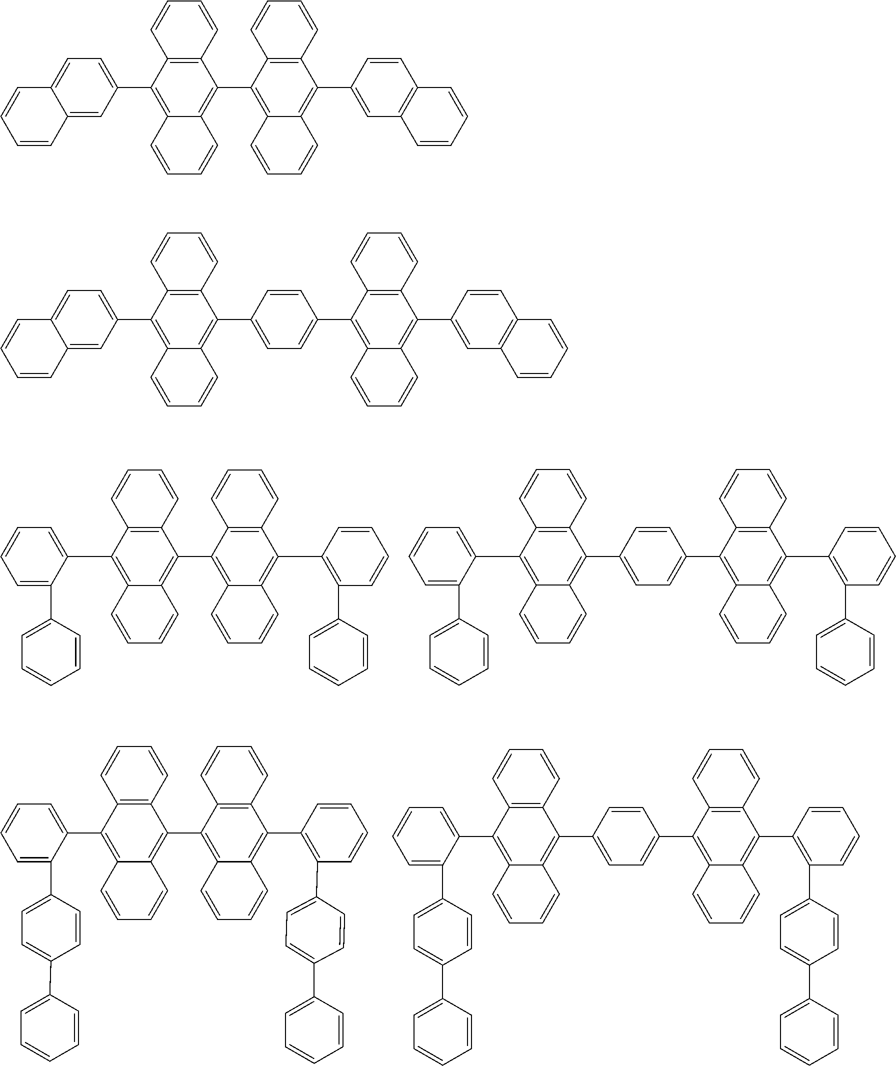

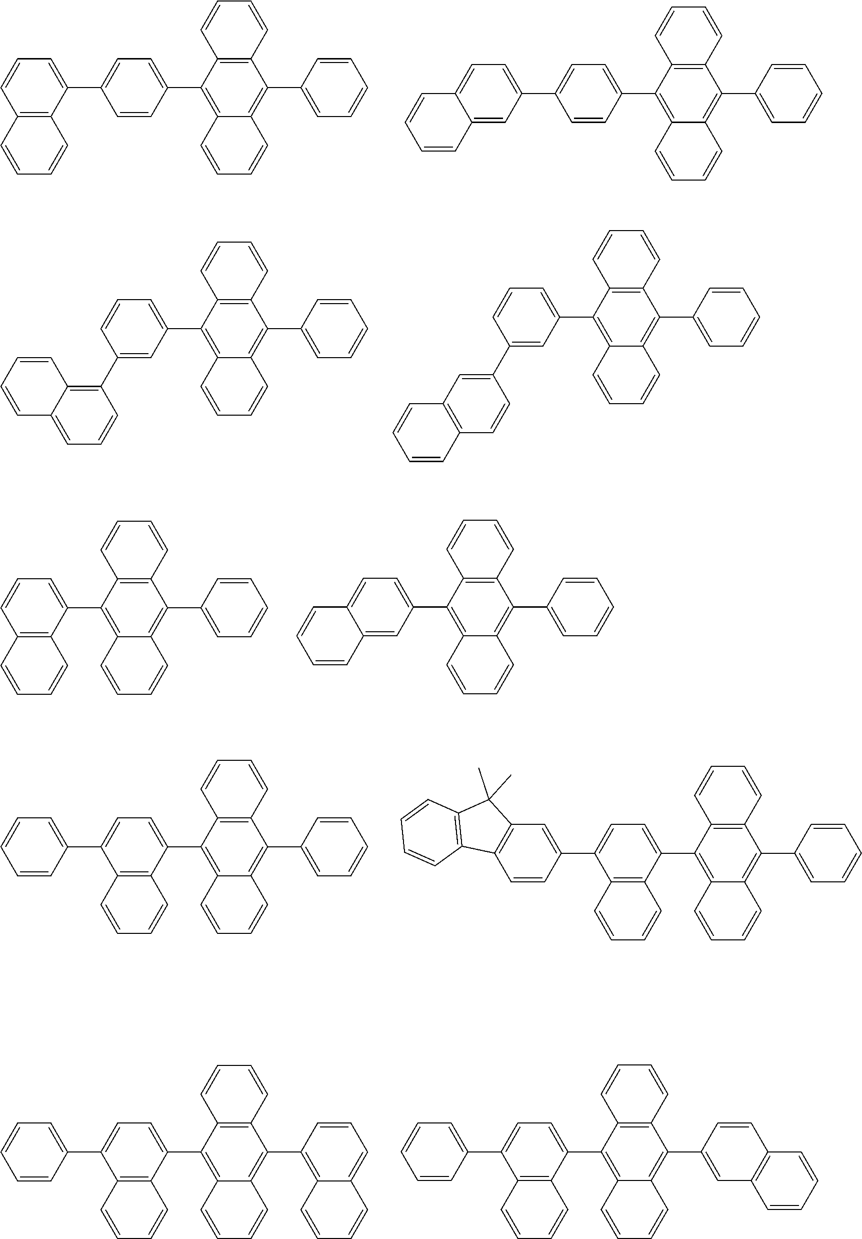

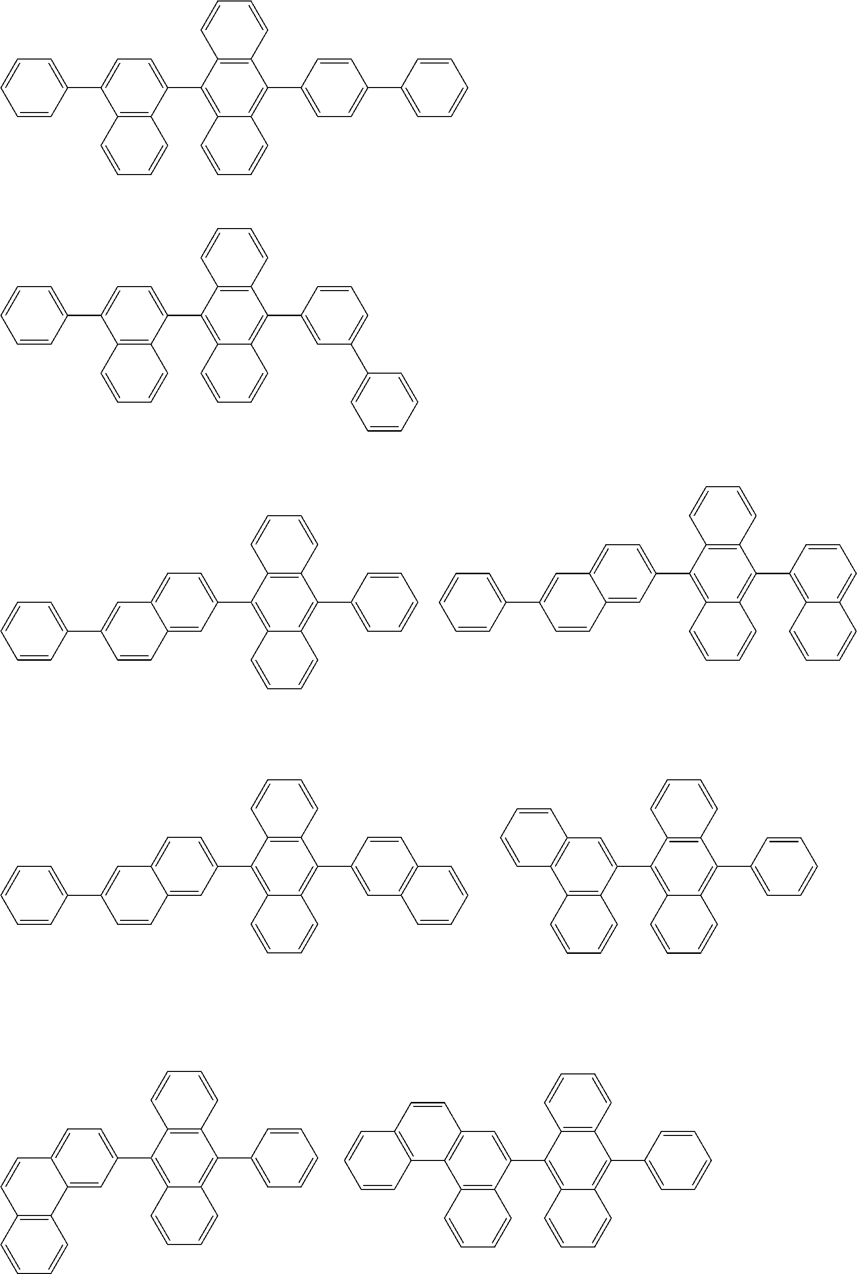

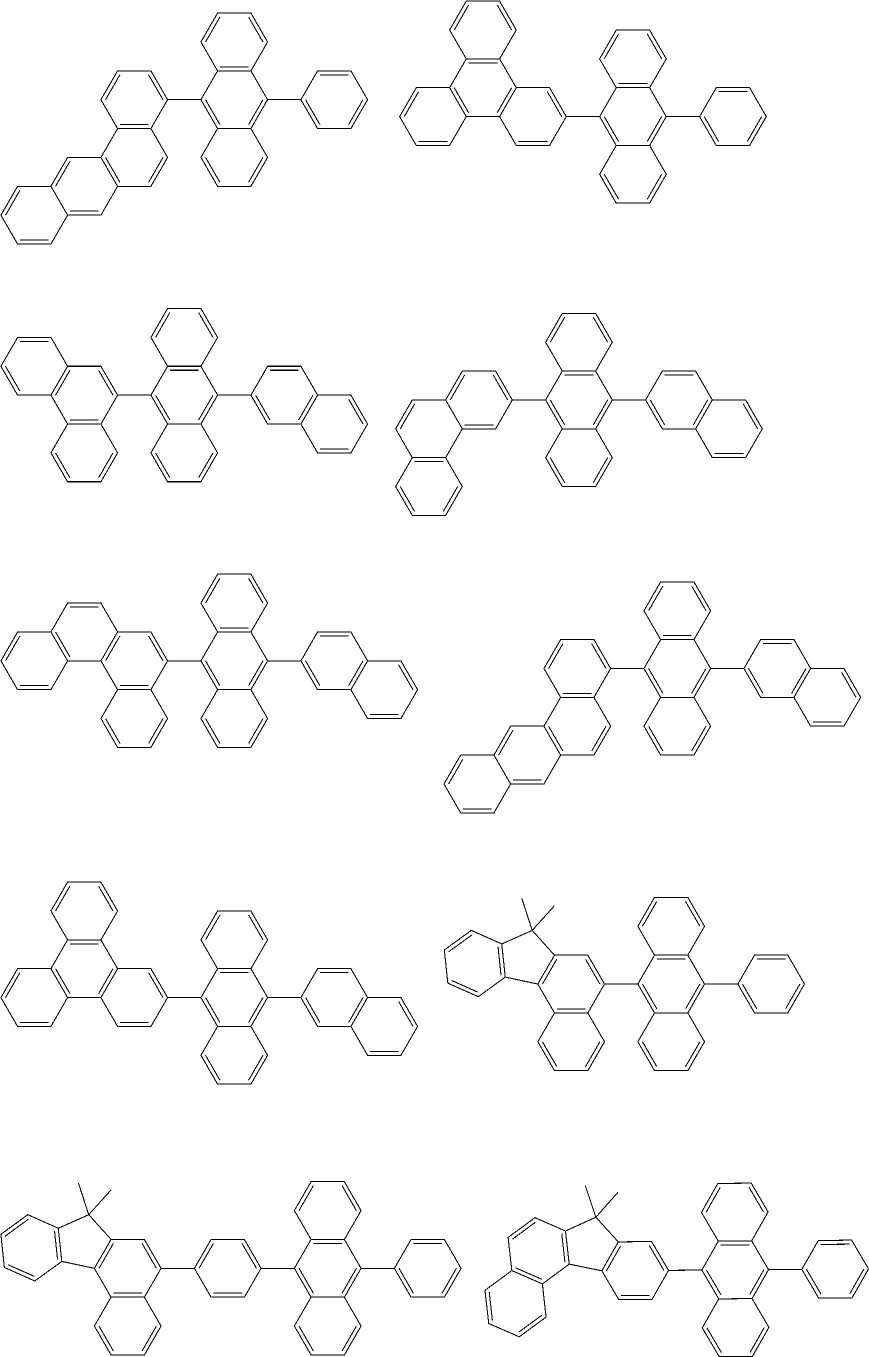

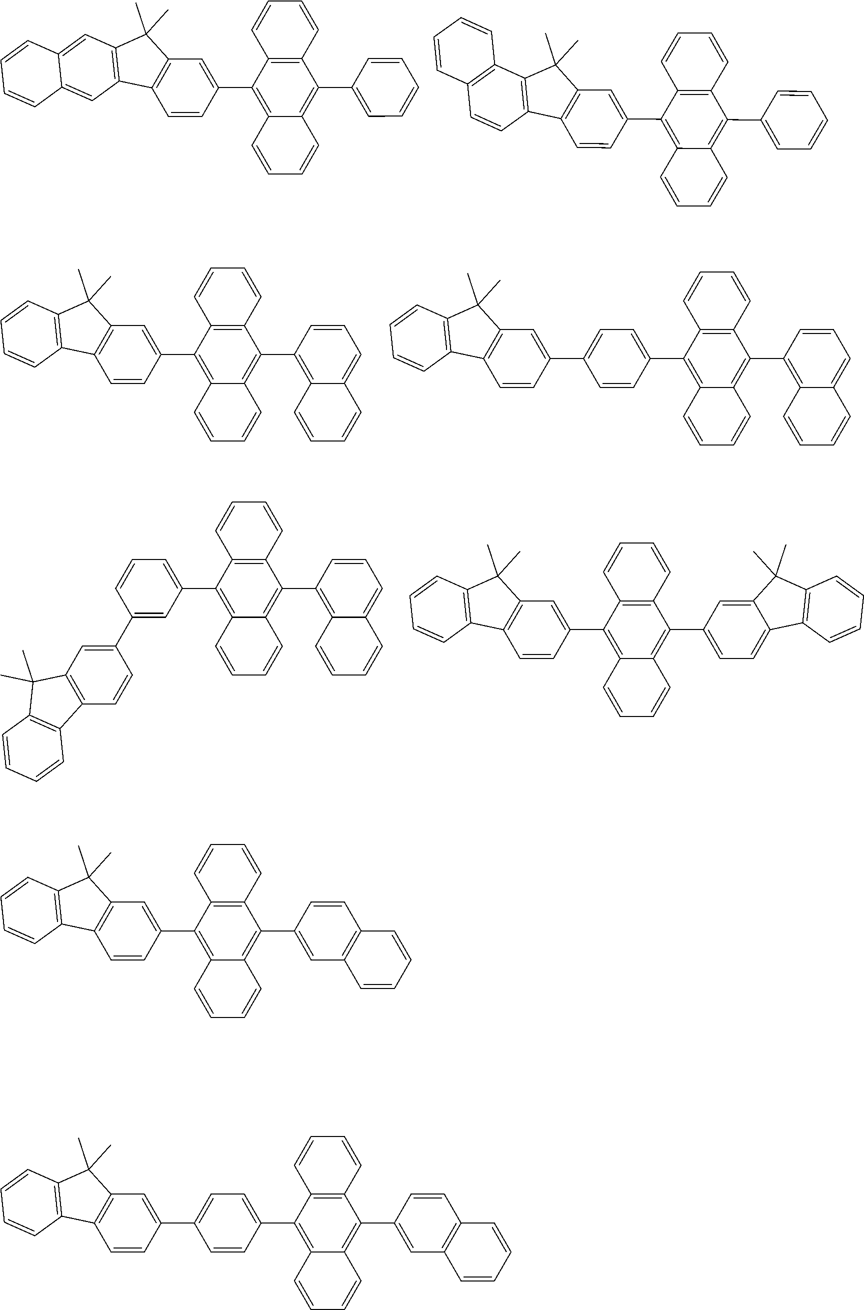

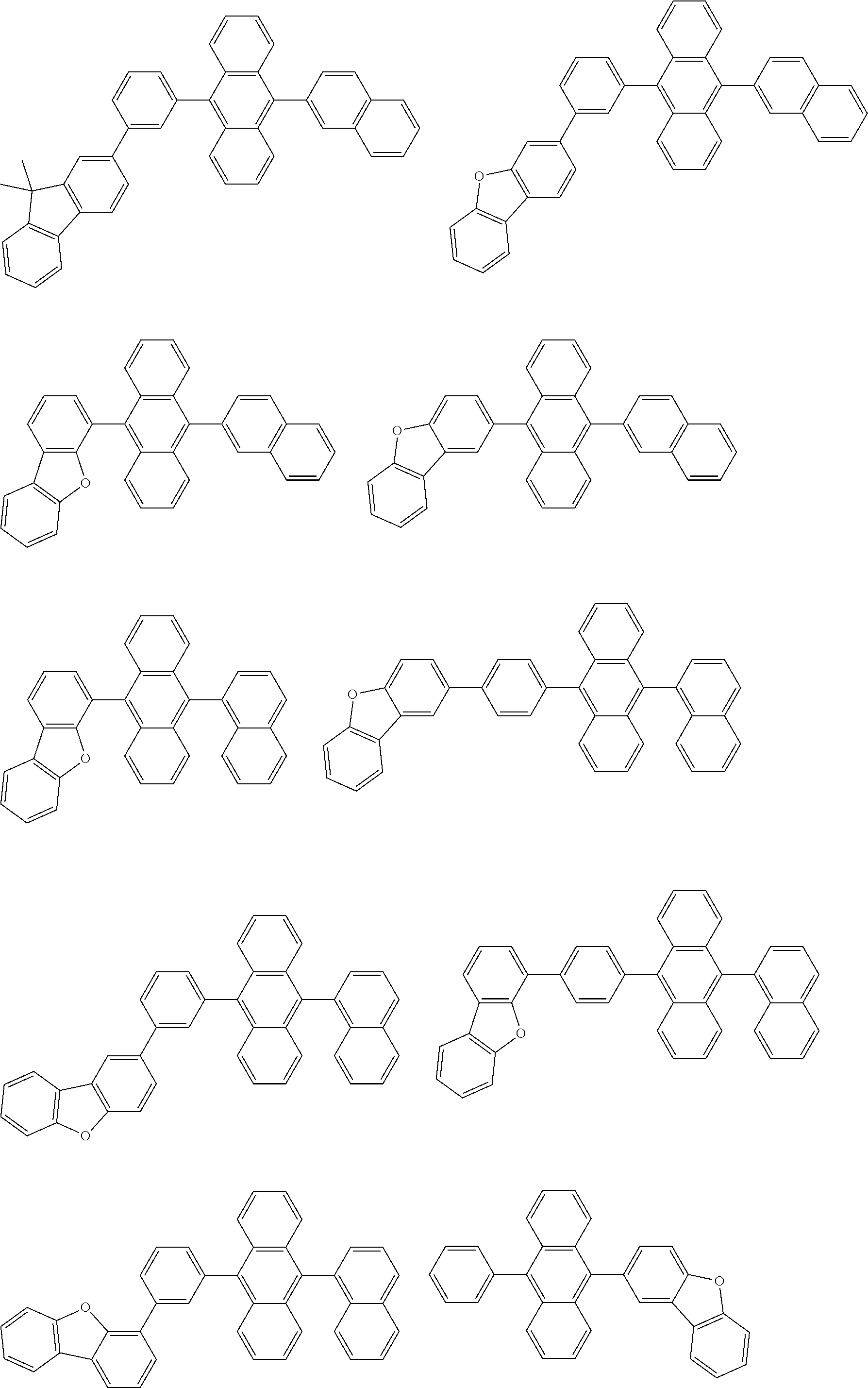

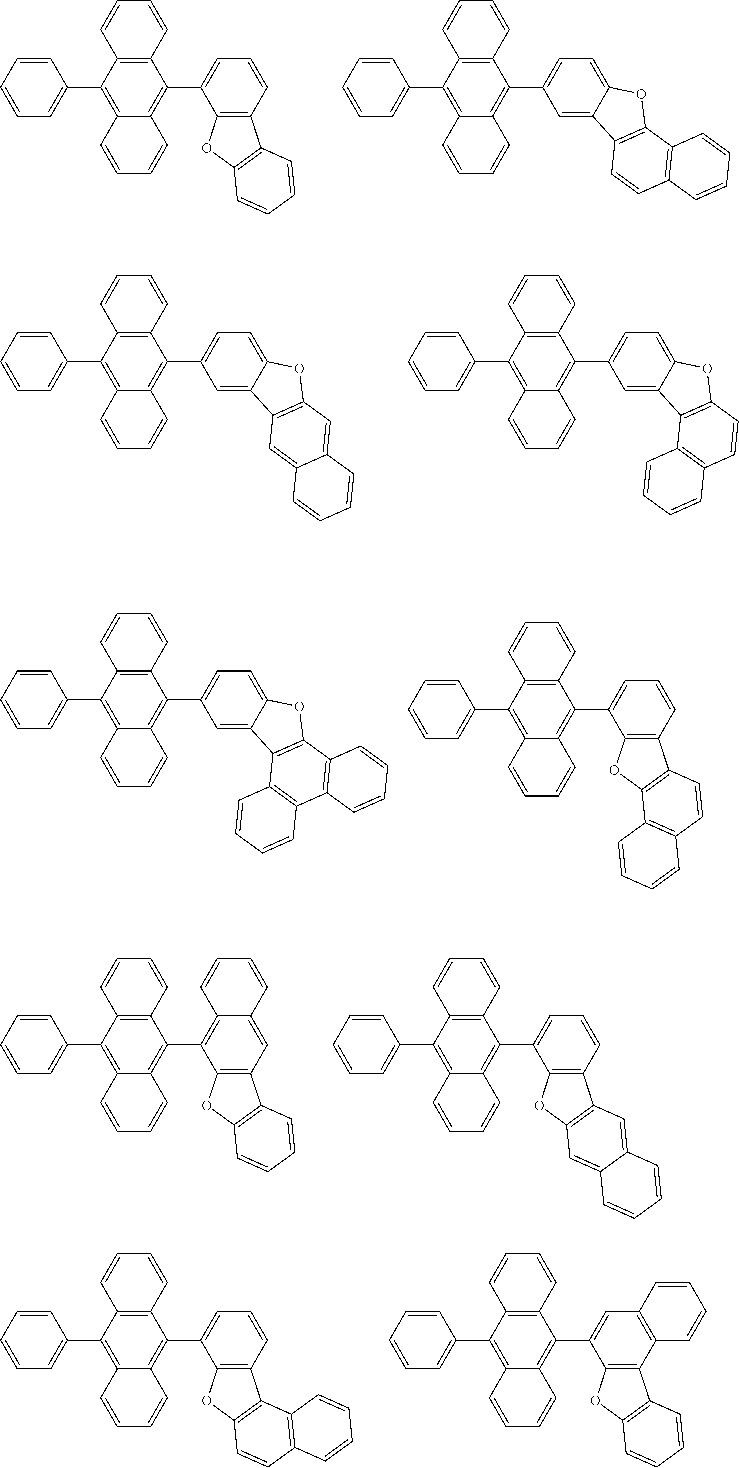

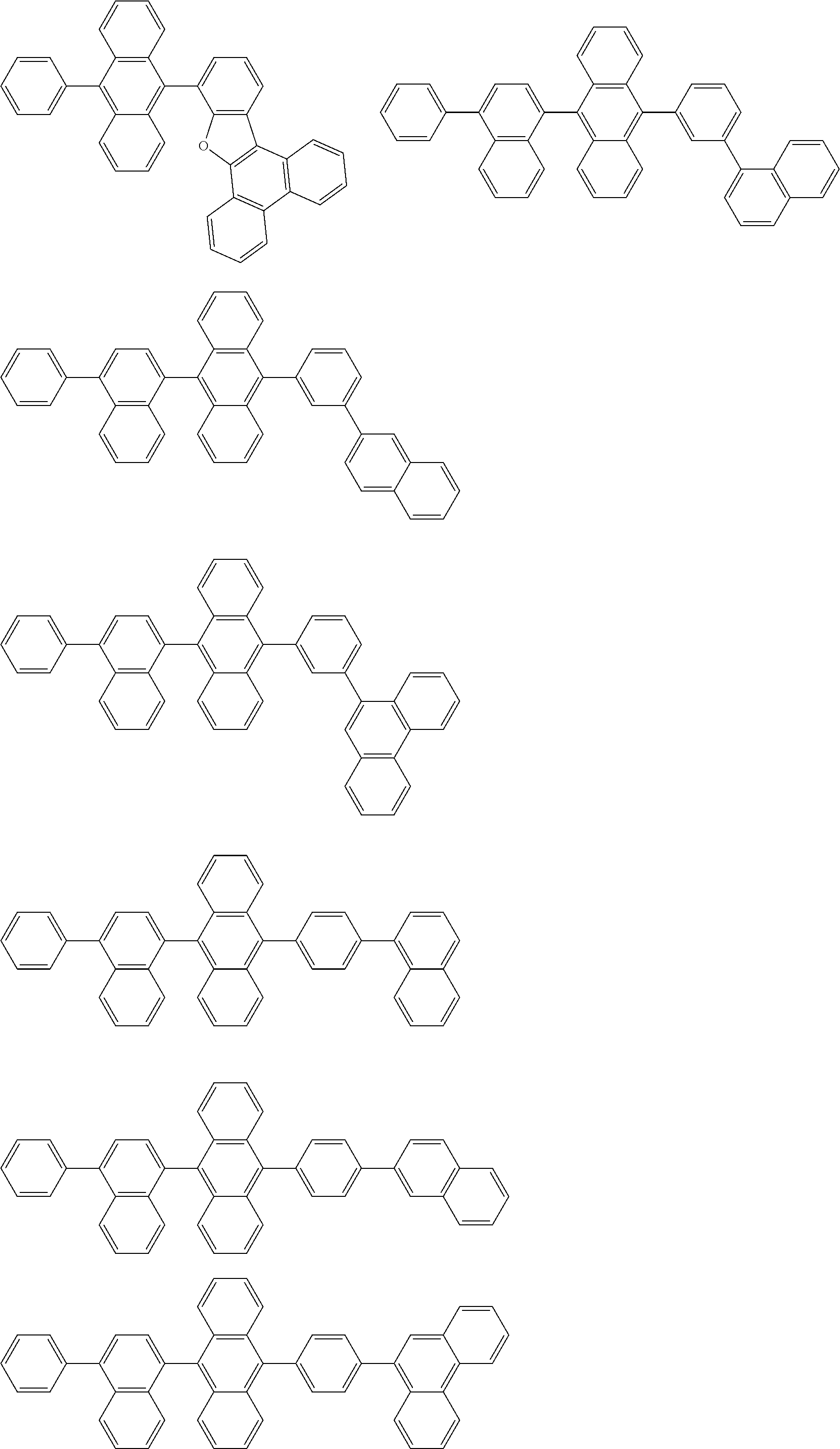

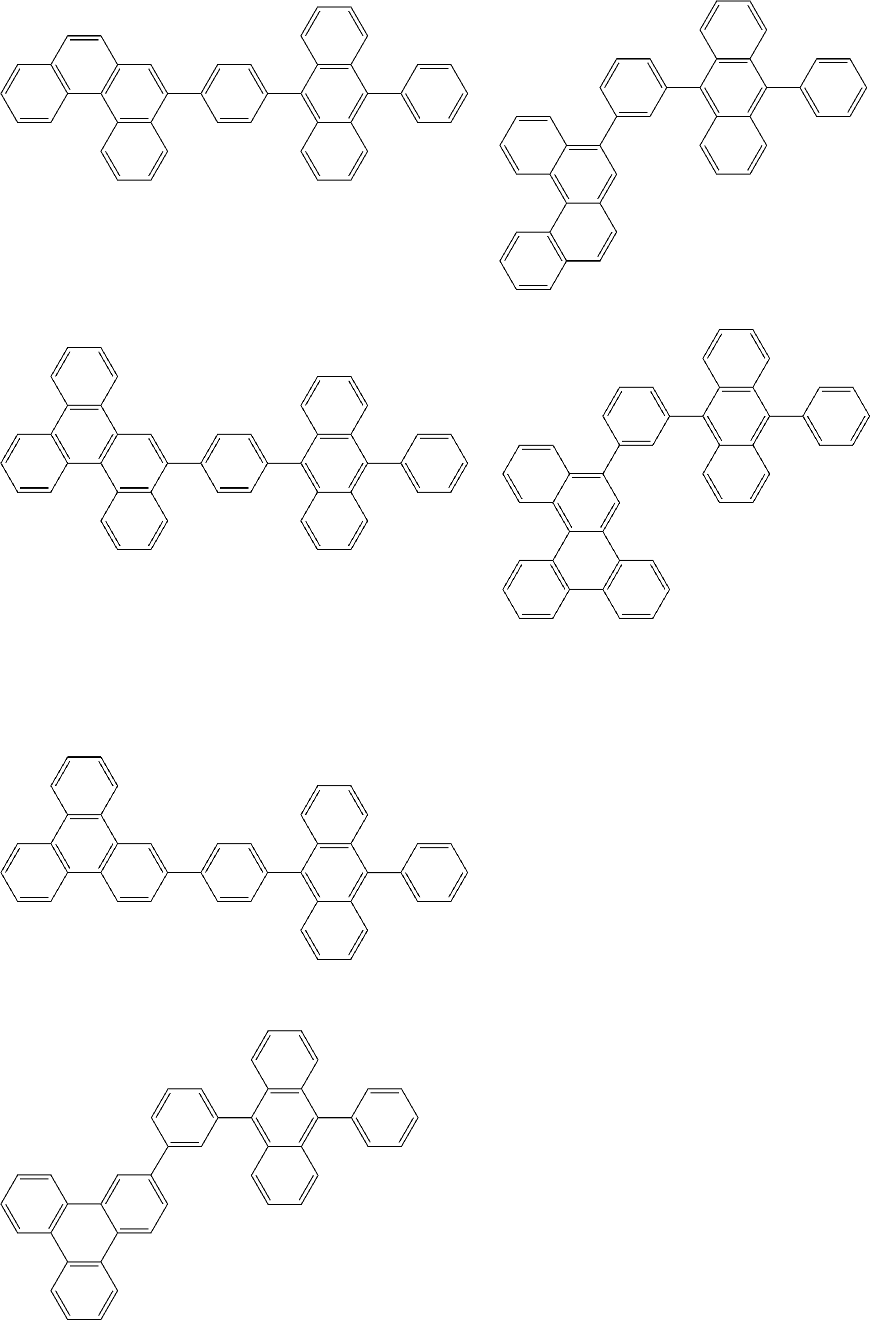

### Electron Transporting Layer

The electron transporting layer is a layer containing a material having a high electron transporting capability (an electron transporting material) and is provided between the light emitting layer and the cathode, or between the electron injecting layer, if existing, and the light emitting layer.

The electron transporting layer may have a single layer structure or a multilayer structure including two or more layers. For example, the electron transporting layer may have a two-layer structure including a first electron transporting layer (anode side) and a second electron transporting layer (cathode side). In one aspect of the present invention, the electron transporting layer having a single layer structure is preferably disposed adjacent to the light emitting layer, and the electron transporting layer that is closest to the anode in the multilayer structure, such as the first electron transporting layer in the two-layer structure, is preferably disposed adjacent to the light emitting layer. In another aspect of the present invention, a hole blocking layer described later can be disposed between the electron transporting layer having a single layer structure and the light emitting layer, or between the electron transporting layer that is closest to the light emitting layer in the multilayer structure and the light emitting layer.

As the electron transporting layer, for example, the followings may be used:
(1) a metal complex, such as an aluminum complex, a beryllium complex, and a zinc complex;
(2) a heteroaromatic compound, such as an imidazole derivative, a benzimidazole derivative, an azine derivative, a carbazole derivative, and a phenanthroline derivative; and
(3) a high-molecular weight compound.

Examples of the metal complex include tris(8-quinolinolato)aluminum(III) (abbreviation: Alq), tris(4-methyl-8-quinolinolato)aluminum (abbreviation: Almq3), bis(10-hydroxybenzo[h]quinolinato)beryllium (abbreviation: BeBq₂), bis(2-methyl-8-quinolinolato)(4-phenylphenolato)aluminum(III) (abbreviation: BAlq), bis(8-quinolinolato)zinc(II) (abbreviation: Znq), bis[2-(2-benzoxazolyl)phenolato]zinc(II) (abbreviation: ZnPBO), bis[2-(2-benzothiazolyl)phenolato]zinc(II) (abbreviation: ZnBTZ), and (8-quinolinolato)lithium (abbreviation: Liq).

Examples of the heteroaromatic compound include 2-(4-biphenylyl)-5-(4-tert-butylphenyl)-1,3,4-oxadiazole (abbreviation: PBD), 1,3-bis[5-(p-tert-butylphenyl)-1,3,4-oxadiazole-2-yl]benzene (abbreviation: OXD-7), 3-(4-tert-butylphenyl)-4-phenyl-5-(4-biphenylyl)-1,2,4-triazole (abbreviation: TAZ), 3-(4-tert-butylphenyl)-4-(4-ethylphenyl)-5-(4-biphenylyl)-1,2,4-triazole (abbreviation: p-EtTAZ), bathophenanthroline (abbreviation: BPhen), bathocuproine (abbreviation: BCP), and 4,4′-bis(5-methylbenzxazol-2-yl)stilbene (abbreviation: BzOs).

Examples of the high-molecular weight compound include poly[(9,9-dihexylfluorene-2,7-diyl)-co-(pyridine-3,5-diyl)] (abbreviation: PF-Py), and poly[(9,9-dioctylfluorene-2,7-diyl)-co-(2,2′-bipyridine-6,6′-diyl)] (abbreviation: PF-BPy).

The aforementioned materials are materials having an electron mobility of 10⁻⁶ cm²/Vs or more. Materials other than those as mentioned above may also be used in the electron transporting layer so long as they are materials high in the electron transporting capability rather than in the hole transporting capability.

### Electron Injecting Layer

The electron injecting layer is a layer containing a material having a high electron injection capability. As the electron injecting layer, alkali metals, such as lithium (Li) and cesium (Cs), alkaline earth metals, such as magnesium (Mg), calcium (Ca), and strontium (Sr), rare earth metals, such as europium (Eu) and ytterbium (Yb), and compounds containing these metals can be used. Examples of the compounds include an alkali metal oxide, an alkali metal halide, an alkali metal-containing organic complex, an alkaline earth metal oxide, an alkaline earth metal halide, an alkaline earth metal-containing organic complex, a rare earth metal oxide, a rare earth metal halide, and a rare earth metal-containing organic complex. These compounds can be used as a mixture of a plurality thereof.

In addition, a material having an electron transporting capability, in which an alkali metal, an alkaline earth metal, or a compound thereof is contained, specifically Alq in which magnesium (Mg) is contained can be used. In this case, electron injection from the cathode can be more efficiently performed.

Alternatively, in the electron injecting layer, a composite material obtained by mixing an organic compound with an electron donor can be used. Such a composite material is excellent in the electron injection capability and the electron transporting capability because the organic compound receives electrons from the electron donor. In this case, the organic compound is preferably a material excellent in transporting received electrons, and specifically, examples thereof include a material constituting the aforementioned electron transporting layer (such as a metal complex and a heteroaromatic compound). As the electron donor, a material having an electron donation property for the organic compound can be used. Specifically, alkali metals, alkaline earth metals, and rare earth metals are preferred, and examples thereof include lithium, cesium, magnesium, calcium, erbium, and ytterbium. In addition, an alkali metal oxide or an alkaline earth metal oxide is preferred, and examples thereof include lithium oxide, calcium oxide, and barium oxide. In addition, a Lewis base, such as magnesium oxide, can also be used. In addition, an organic compound, such as tetrathiafulvalene (abbreviation: TTF), can also be used.

### Cathode

It is preferred that a metal, an alloy, an electrically conductive compound, or a mixture thereof which has a low work function (specifically 3.8 eV or less) is used for the cathode. Specific examples of such a cathode material include elements belonging to group 1 or 2 of the periodic table of the elements, that is, alkali metals, such as lithium (Li) and cesium (Cs), alkaline earth metals, such as magnesium (Mg), calcium (Ca), and strontium (Sr), and alloys containing these (such as MgAg, and AlLi), and rare earth metals, such as europium (Eu), and ytterbium (Yb) and alloys containing these.

When the cathode is formed by using the alkali metals, the alkaline earth metals, and the alloys containing these, a vacuum vapor deposition method or a sputtering method can be adopted. In addition, when a silver paste or the like is used, a coating method, an inkjet method, or the like can be adopted.

By providing the electron injecting layer, the cathode can be formed using various conductive materials, such as Al, Ag, ITO, graphene, and indium oxide-tin oxide containing silicon or silicon oxide regardless of the magnitude of a work function. Such a conductive material can be deposited by using a sputtering method, an inkjet method, a spin coating method, or the like.

### Insulating Layer

Since an electric field is applied to an ultrathin film, pixel defects are likely to occur to the organic EL device due to leaks or short-circuiting. In order to prevent this, an insulating layer formed of an insulating thin film layer can be inserted between a pair of electrodes.

Examples of the material used for the insulating layer include aluminum oxide, lithium fluoride, lithium oxide, cesium fluoride, cesium oxide, magnesium oxide, magnesium fluoride, calcium oxide, calcium fluoride, aluminum nitride, titanium oxide, silicon oxide, germanium oxide, silicon nitride, boron nitride, molybdenum oxide, ruthenium oxide, and vanadium oxide. A mixture or a laminate of these may also be used.

### Space Layer

The space layer is, for example, a layer provided between a fluorescent light emitting layer and a phosphorescent light emitting layer for the purpose of preventing excitons generated in the phosphorescent light emitting layer from diffusing into the fluorescent light emitting layer, or adjusting a carrier balance, in the case where the fluorescent light emitting layers and the phosphorescent light emitting layers are stacked. The space layer can also be provided among the plurality of phosphorescent light emitting layers.

Since the space layer is provided between the light emitting layers, a material having both an electron transporting capability and a hole transporting capability is preferred. Also, one having a triplet energy of 2.6 eV or more is preferred in order to prevent triplet energy diffusion in the adjacent phosphorescent light emitting layer. Examples of the material used for the space layer include the same as those used for the hole transporting layer as described above.

### Blocking Layer

The blocking layer such as the electron blocking layer, the hole blocking layer, or the exciton blocking layer can be provided adjacent to the light emitting layer. The electron blocking layer is a layer that prevents electrons from leaking from the light emitting layer to the hole transporting layer, and the hole blocking layer is a layer that prevents holes from leaking from the light emitting layer to the electron transporting layer. The exciton blocking layer has a function of preventing excitons generated in the light emitting layer from diffusing into the surrounding layers, and trapping the excitons within the light emitting layer.

Each layer of the organic EL device can be formed by a conventionally known vapor deposition method, a coating method, or the like. For example, formation can be performed by a known method using a vapor deposition method such as a vacuum vapor deposition method, or a molecular beam vapor deposition method (MBE method), or a coating method using a solution of a compound for forming a layer, such as a dipping method, a spin-coating method, a casting method, a bar-coating method, and a roll-coating method.

The film thickness of each layer is not particularly limited, but is typically 5 nm to 10 μm, and more preferably 10 nm to 0.2 μm because in general, when the film thickness is too small, defects such as pinholes are likely to occur, and conversely, when the film thickness is too large, a high driving voltage is required and the efficiency decreases.

The organic EL device can be used for electronic devices, such as display components of an organic EL panel module, display devices of a television, a mobile phone and a personal computer, and light emitting devices of lightings and vehicular lamps.

### EXAMPLES

The present invention is hereunder described in more detail by reference to Examples, but it should be construed that the present invention is not limited to the following Examples.

### Compound Used for Production of Organic EL Device of Example 1

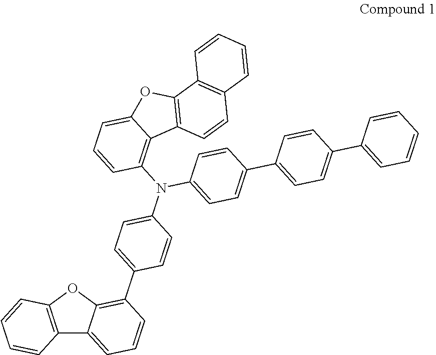 [SMILES: c1ccc(-c2ccc(-c3ccc(N(c4ccc(-c5cccc6c5oc5ccccc56)cc4)c4cccc5oc6c7ccccc7ccc6c45)cc3)cc2)cc1]

### Compounds Used for Production of Organic EL Devices of Examples 2 to 7

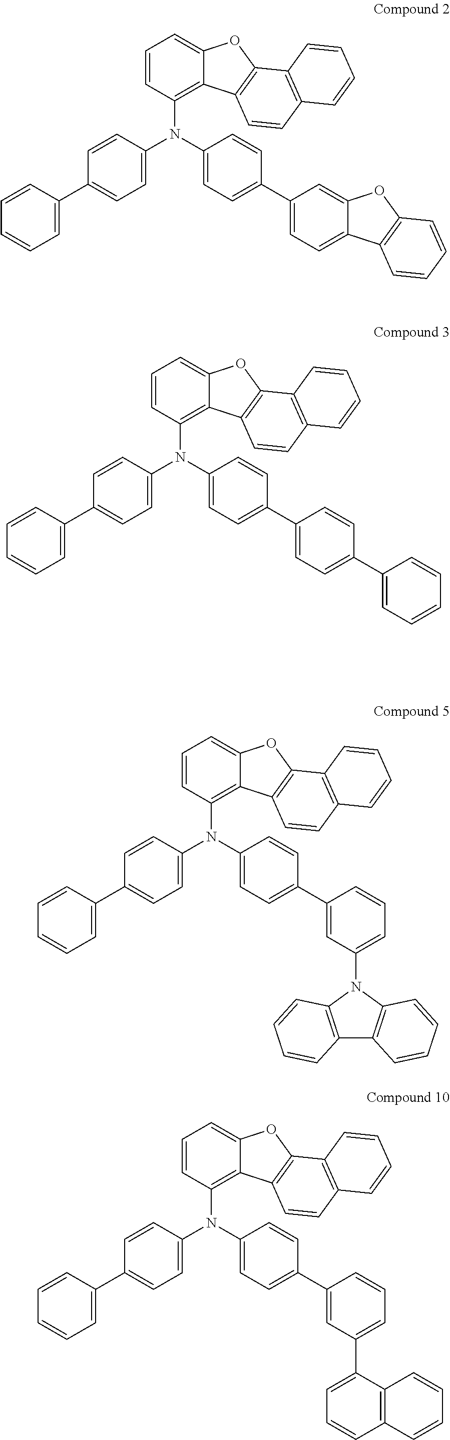 [SMILES: c1ccc(-c2ccc(N(c3ccc(-c4ccc5c(c4)oc4ccccc45)cc3)c3cccc4oc5c6ccccc6ccc5c34)cc2)cc1 ; c1ccc(-c2ccc(-c3ccc(N(c4ccc(-c5ccccc5)cc4)c4cccc5oc6c7ccccc7ccc6c45)cc3)cc2)cc1 ; c1ccc(-c2ccc(N(c3ccc(-c4cccc(-n5c6ccccc6c6ccccc65)c4)cc3)c3cccc4oc5c6ccccc6ccc5c34)cc2)cc1 ; c1ccc(-c2ccc(N(c3ccc(-c4cccc(-c5cccc6ccccc56)c4)cc3)c3cccc4oc5c6ccccc6ccc5c34)cc2)cc1]

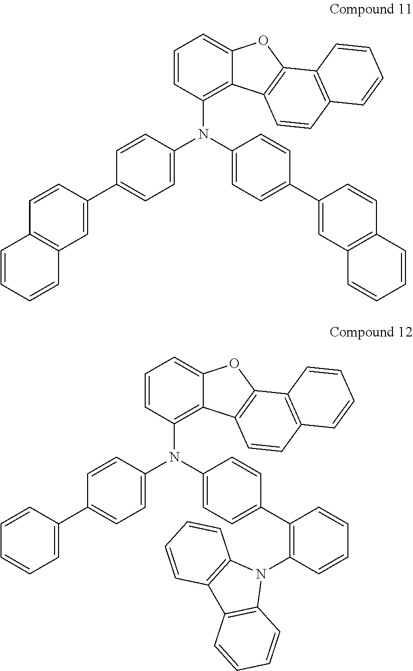 [SMILES: c1ccc2cc(-c3ccc(N(c4ccc(-c5ccc6ccccc6c5)cc4)c4cccc5oc6c7ccccc7ccc6c45)cc3)ccc2c1 ; c1ccc(-c2ccc(N(c3ccc(-c4ccccc4-n4c5ccccc5c5ccccc54)cc3)c3cccc4oc5c6ccccc6ccc5c34)cc2)cc1]

### Compounds Used for Production of Organic EL Devices of Examples 8 to 17

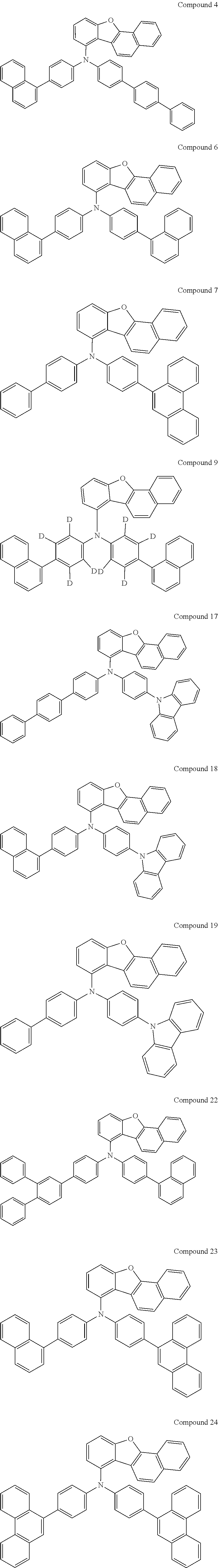

### Compounds Used for Production of Organic EL Devices of Examples 18 to 22

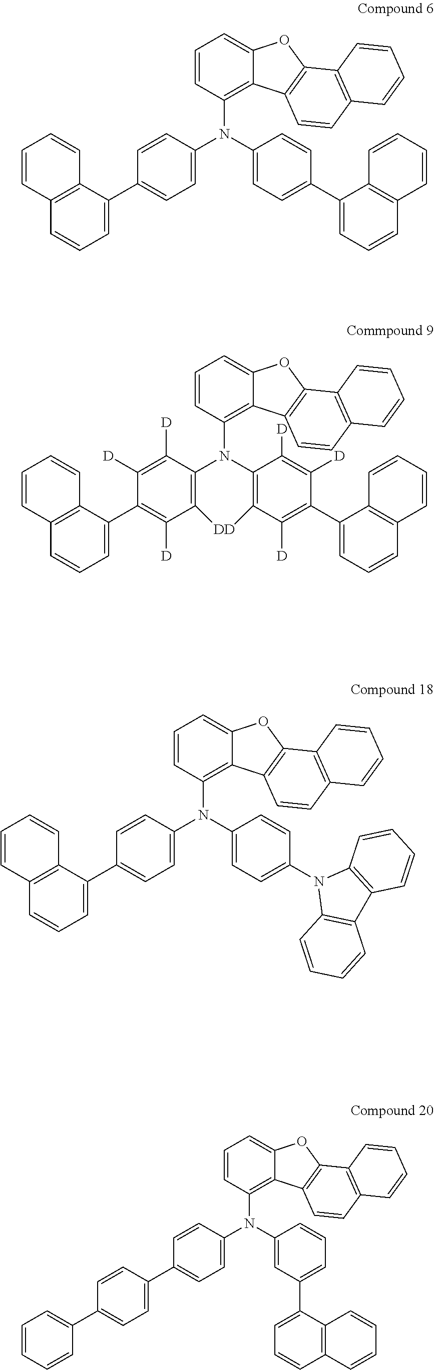 [SMILES: c1ccc2c(-c3ccc(N(c4ccc(-c5cccc6ccccc56)cc4)c4cccc5oc6c7ccccc7ccc6c45)cc3)cccc2c1 ; [2H]c1c([2H])c(N(c2c([2H])c([2H])c(-c3cccc4ccccc34)c([2H])c2[2H])c2cccc3oc4c5ccccc5ccc4c23)c([2H])c([2H])c1-c1cccc2ccccc12 ; c1ccc2c(-c3ccc(N(c4ccc(-n5c6ccccc6c6ccccc65)cc4)c4cccc5oc6c7ccccc7ccc6c45)cc3)cccc2c1 ; c1ccc(-c2ccc(-c3ccc(N(c4cccc(-c5cccc6ccccc56)c4)c4cccc5oc6c7ccccc7ccc6c45)cc3)cc2)cc1]

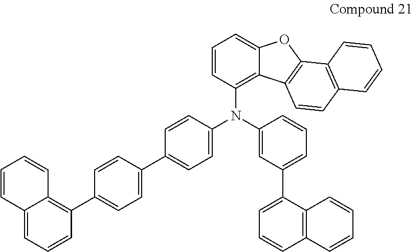 [SMILES: c1cc(-c2cccc3ccccc23)cc(N(c2ccc(-c3ccc(-c4cccc5ccccc45)cc3)cc2)c2cccc3oc4c5ccccc5ccc4c23)c1]

### Comparative Compound Used for Production of Organic EL Devices of Comparative Example 1

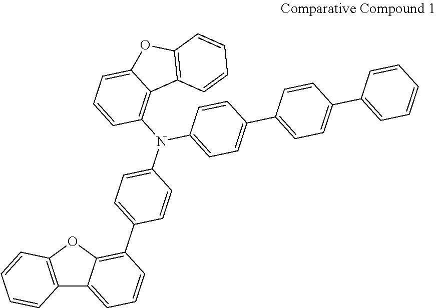 [SMILES: c1ccc(-c2ccc(-c3ccc(N(c4ccc(-c5cccc6c5oc5ccccc56)cc4)c4cccc5oc6ccccc6c45)cc3)cc2)cc1]

### Comparative Compounds Used for Production of Organic EL Devices of Comparative Examples 2 to 5

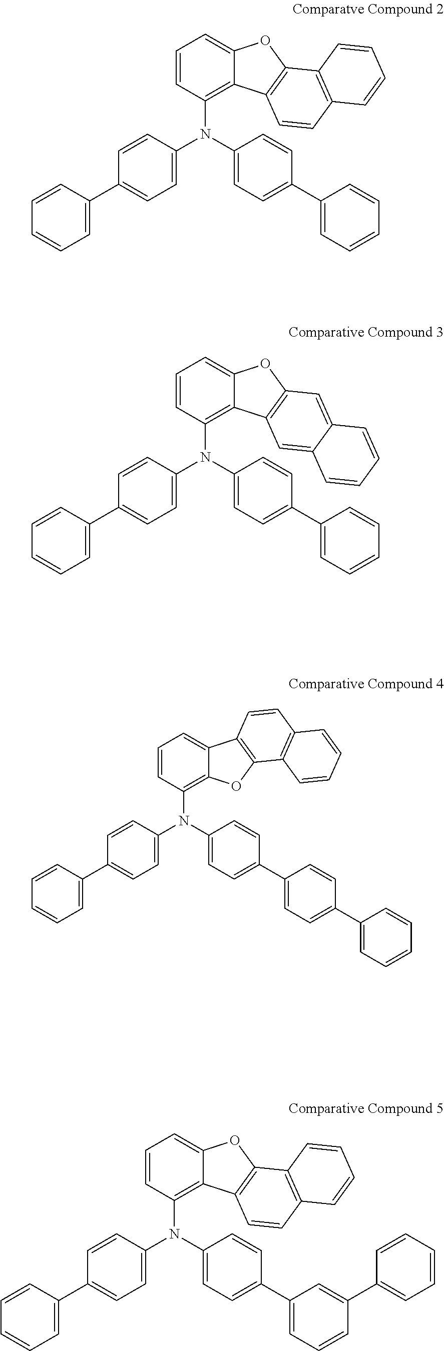 [SMILES: c1ccc(-c2ccc(N(c3ccc(-c4ccccc4)cc3)c3cccc4oc5c6ccccc6ccc5c34)cc2)cc1 ; c1ccc(-c2ccc(N(c3ccc(-c4ccccc4)cc3)c3cccc4oc5cc6ccccc6cc5c34)cc2)cc1 ; c1ccc(-c2ccc(-c3ccc(N(c4ccc(-c5ccccc5)cc4)c4cccc5c4oc4c6ccccc6ccc54)cc3)cc2)cc1 ; c1ccc(-c2ccc(N(c3ccc(-c4cccc(-c5ccccc5)c4)cc3)c3cccc4oc5c6ccccc6ccc5c34)cc2)cc1]

### Comparative Compounds Used for Production of Organic EL Devices of Comparative Examples 6 and 7

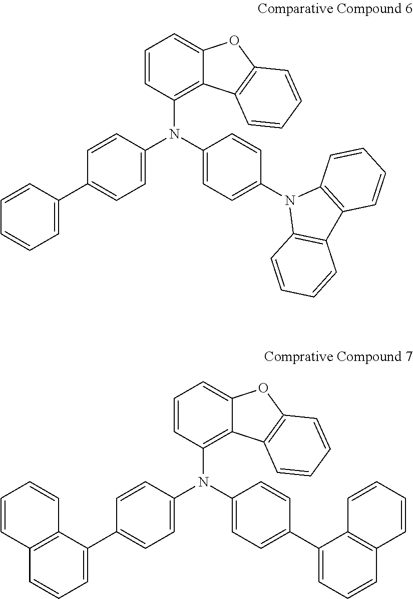 [SMILES: c1ccc(-c2ccc(N(c3ccc(-n4c5ccccc5c5ccccc54)cc3)c3cccc4oc5ccccc5c34)cc2)cc1 ; c1ccc2c(-c3ccc(N(c4ccc(-c5cccc6ccccc56)cc4)c4cccc5oc6ccccc6c45)cc3)cccc2c1]

### Comparative Compound Used for Production of Organic EL Devices of Comparative Example 8

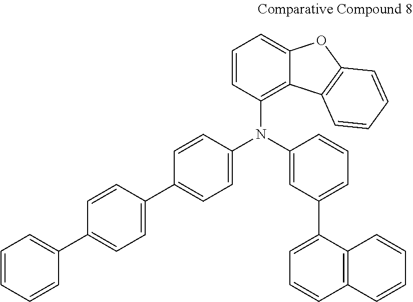 [SMILES: c1ccc(-c2ccc(-c3ccc(N(c4cccc(-c5cccc6ccccc56)c4)c4cccc5oc6ccccc6c45)cc3)cc2)cc1]

### Other Compounds Used for Production of Organic EL Devices of Example 1 and Comparative Example 1

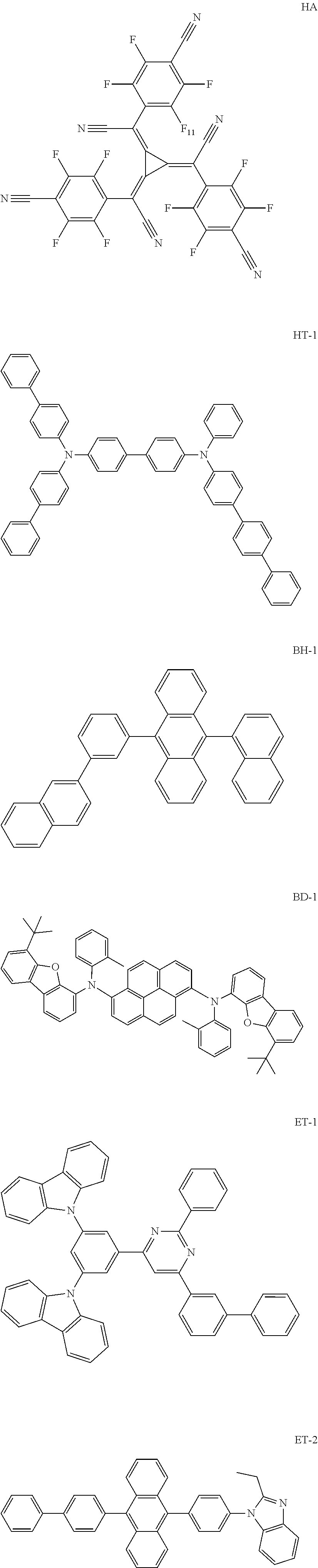

### Other Compounds Used for Production of Organic EL Devices of Examples 2 to 7 and Comparative Examples 2 to 5

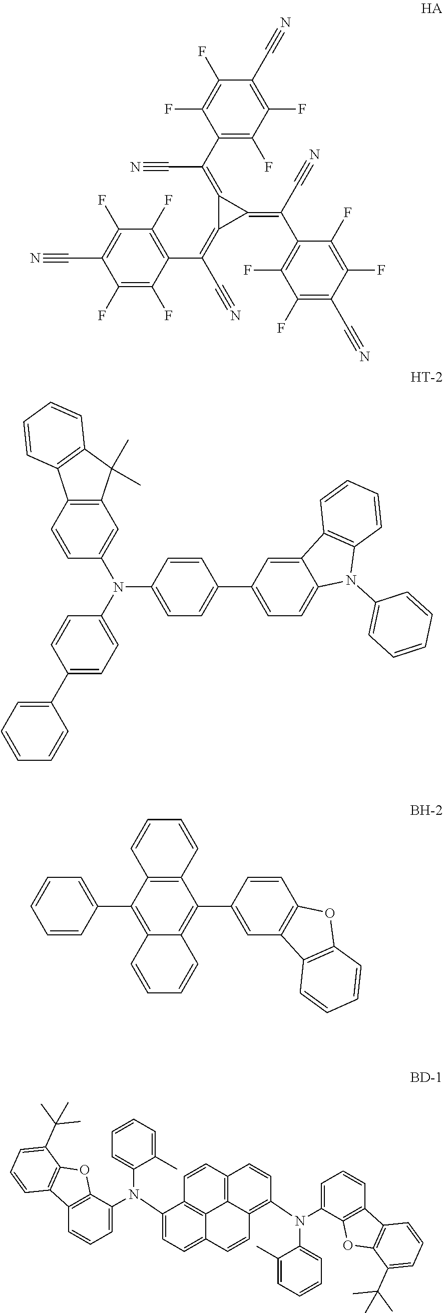 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(-c2c3ccccc3c(-c3ccc4oc5ccccc5c4c3)c3ccccc23)cc1 ; Cc1ccccc1N(c1ccc2ccc3c(N(c4ccccc4C)c4cccc5c4oc4c(C(C)(C)C)cccc45)ccc4ccc1c2c43)c1cccc2c1oc1c(C(C)(C)C)cccc12]

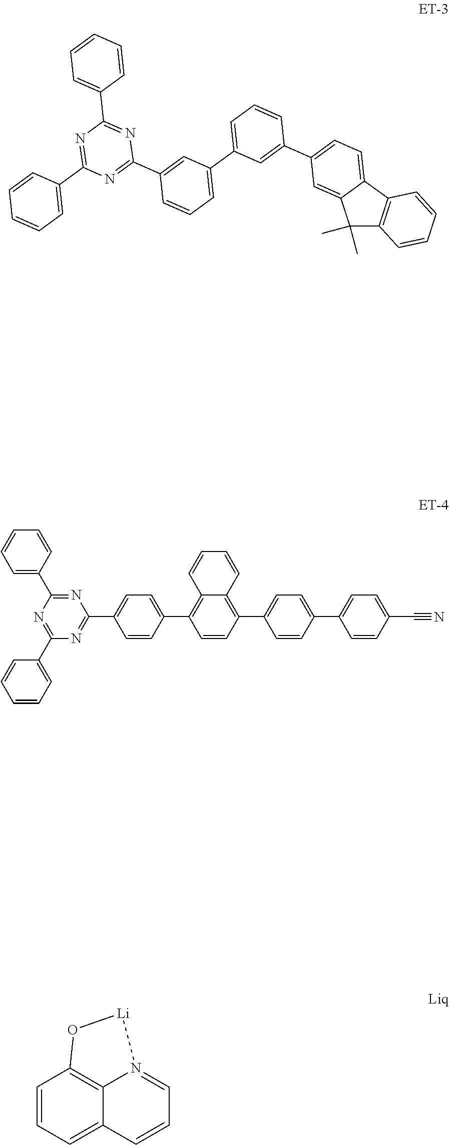

### Other Compounds Used for Production of Organic EL Devices of Examples 8 to 17 and Comparative Examples 6 and 7

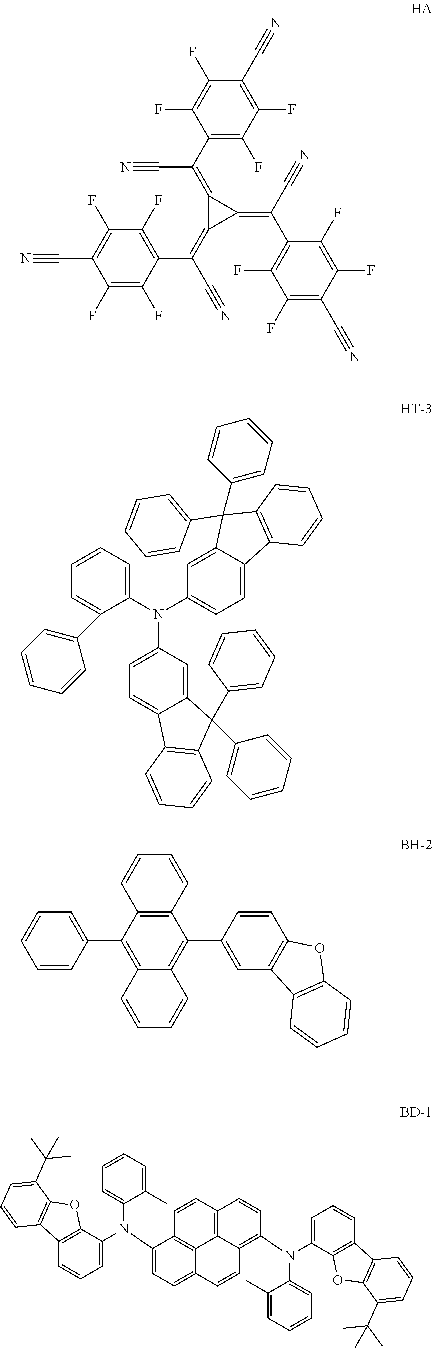 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; c1ccc(-c2ccccc2N(c2ccc3c(c2)C(c2ccccc2)(c2ccccc2)c2ccccc2-3)c2ccc3c(c2)C(c2ccccc2)(c2ccccc2)c2ccccc2-3)cc1 ; c1ccc(-c2c3ccccc3c(-c3ccc4oc5ccccc5c4c3)c3ccccc23)cc1 ; Cc1ccccc1N(c1ccc2ccc3c(N(c4ccccc4C)c4cccc5c4oc4c(C(C)(C)C)cccc45)ccc4ccc1c2c43)c1cccc2c1oc1c(C(C)(C)C)cccc12]

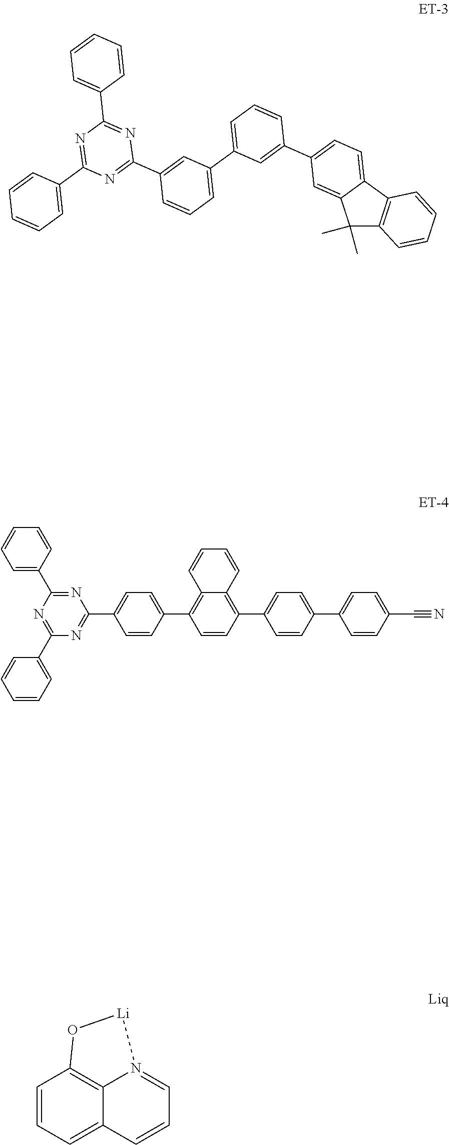

### Other Compounds Used for Production of Organic EL Devices of Examples 18 to 22 and Comparative Example 8

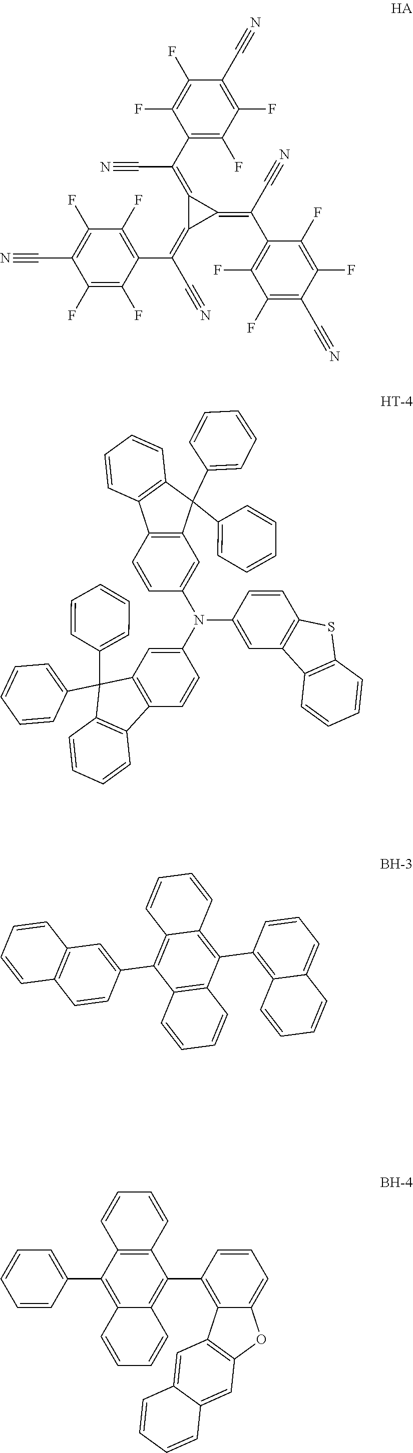

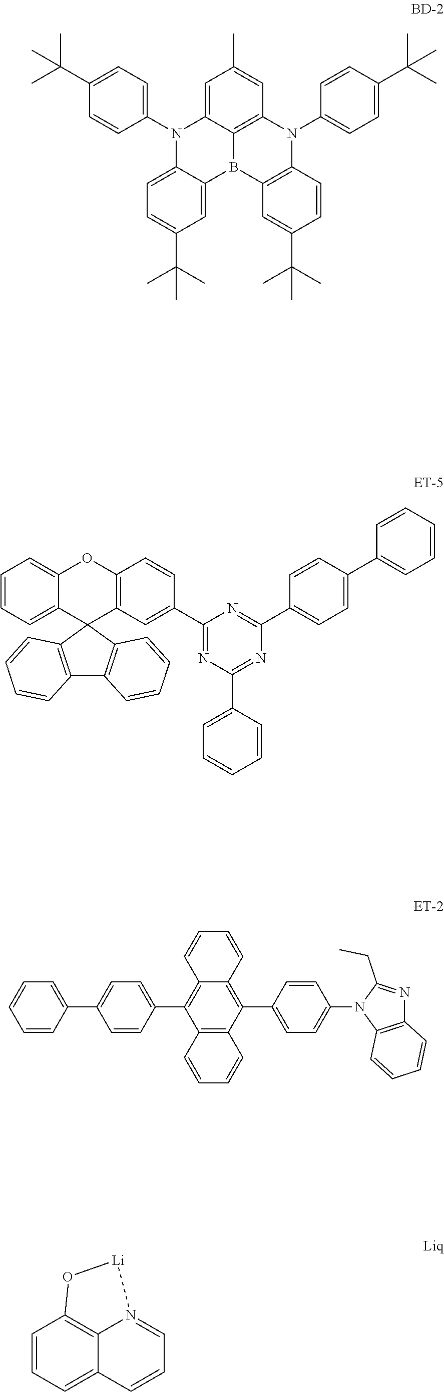

### Production of Organic EL Device

### Example 1

A glass substrate of 25 mm×75 mm×1.1 mm provided with an ITO transparent electrode (anode) (manufactured by GEOMATEC Co., Ltd.) was ultrasonically cleaned in isopropyl alcohol for 5 minutes and then subjected to UV ozone cleaning for 30 minutes. The film thickness of the ITO was 130 nm.

The cleaned glass substrate provided with the transparent electrode was mounted on a substrate holder of a vacuum vapor deposition apparatus, and firstly, Compound HT-1 and Compound HA were vapor co-deposited on the surface having the transparent electrode formed thereon, so as to cover the transparent electrode, resulting in a hole injecting layer with a film thickness of 10 nm. The mass ratio of Compound HT-1 to Compound HA (HT-1/HA) was 97/3.

Subsequently, on this hole injecting layer, Compound HT-1 was vapor deposited to form a first hole transporting layer with a film thickness of 80 nm.

Subsequently, on this first hole transporting layer, Compound 1 was vapor deposited to form a second hole transporting layer with a film thickness of 10 nm.

Subsequently, on this second hole transporting layer, Compound BH-1 (host material) and Compound BD-1 (dopant material) were vapor co-deposited to form a light emitting layer with a film thickness of 25 nm. The mass ratio of Compound BH-1 to Compound BD-1 (BH-1/BD-1) was 96/4.

Subsequently, on this light emitting layer, Compound ET-1 was vapor deposited to form a first electron transporting layer with a film thickness of 10 nm.

Subsequently, on this first electron transporting layer, Compound ET-2 was vapor-deposited to form a second electron transporting layer with a film thickness of 15 nm.

Subsequently, on this second electron transporting layer, LiF was vapor deposited to form an electron injecting electrode with a film thickness of 1 nm.

Then, on this electron injecting electrode, metal Al was vapor deposited to form a metal cathode with a film thickness of 50 nm.

The layer configuration of the organic EL device of Example 1 thus obtained is shown as follows:

ITO(130)/HT-1:HA=97:3(10)/HT-1(80)/Compound 1(10)/BH-1:BD-1=96:4(25)/ET-1(10)/ET-2(15)/LiF(1)/Al(50)

In the layer configuration, the numeral in parentheses indicates the film thickness (nm), and the ratio is a mass ratio. The same shall apply to the following Examples and Comparative Examples.

### Comparative Example 1

An organic EL device was produced in the same manner as in Example 1 except that the second hole transporting layer material was changed to Comparative Example 1, as in Table 1 below.

### Evaluation of Organic EL Device

### Lifetime Measurement

A voltage was applied to the resultant organic EL device so that the current density could be 50 mA/cm² to measure the 95% lifetime (LT95) of the organic EL device. Here, the 95% lifetime (LT95) means a period of time (hr) until the luminance of the device decreased to 95% of the initial luminance in constant current driving.

The results are shown in Table 1.

TABLE 1

| | Second Hole Transporting | |
|---|---|---|
| | Layer Material | LT95 [h] |
| Example 1 | Compound 1 | 120 |
| Comparative Example 1 | Comparative Compound 1 | 96 |

As obvious from the results in Table 1, it is known that the monoamine (Compound 1 in Example 1) satisfying the definition in the present invention exhibits an extremely improved value of LT95, as compared with the monoamine (Comparative Compound 1 in Comparative Example 1) not satisfying the definition in the present invention.

### Example 2

A glass substrate of 25 mm×75 mm×1.1 mm provided with an ITO transparent electrode (anode) (manufactured by GEOMATEC Co., Ltd.) was ultrasonically cleaned in isopropyl alcohol for 5 minutes and then subjected to UV ozone cleaning for 30 minutes. The film thickness of the ITO was 130 nm.

The cleaned glass substrate provided with the transparent electrode was mounted on a substrate holder of a vacuum vapor deposition apparatus, and firstly, Compound HT-2 and Compound HA were vapor co-deposited on the surface having the transparent electrode formed thereon, so as to cover the transparent electrode, resulting in a hole injecting layer with a film thickness of 10 nm. The mass ratio of Compound HT-2 to Compound HA (HT-2/HA) was 97/3.

Subsequently, on this hole injecting layer, Compound HT-2 was vapor deposited to form a first hole transporting layer with a film thickness of 80 nm.

Subsequently, on this first hole transporting layer, Compound 2 was vapor deposited to form a second hole transporting layer with a film thickness of 10 nm.

Subsequently, on this second hole transporting layer, Compound BH-2 (host material) and Compound BD-1 (dopant material) were vapor co-deposited to form a light emitting layer with a film thickness of 25 nm. The mass ratio of Compound BH-2 to Compound BD-1 (BH-2/BD-1) was 96/4.

Subsequently, on this light emitting layer, Compound ET-3 was vapor deposited to form a first electron transporting layer with a film thickness of 5 nm.

Subsequently, on this first electron transporting layer, Compound ET-4 and Liq were vapor co-deposited to form a second electron transporting layer with a film thickness of 20 nm.

The mass ratio of Compound ET-4 to Liq (ET-4/Liq) was 50/50.

Subsequently, on this second electron transporting layer, LiF was vapor deposited to form an electron injecting electrode with a film thickness of 1 nm.

Then, on this electron injecting electrode, metal Al was vapor deposited to form a metal cathode with a film thickness of 50 nm.

The layer configuration of the organic EL device of Example 2 thus obtained is shown as follows:

ITO(130)/HT-2:HA=97:3(10)/HT-2(80)/Compound 2(10)/BH-2:BD-1=96:4 (25)/ET-3(5)/ET-4:Liq=50:50(20)/LiF(1)/Al(50)

### Examples 3 to 7

Organic EL devices were produced in the same manner as in Example 2 except that the second hole transporting layer material was changed to Compounds 3, 5, and 10 to 12, respectively, as shown in Table 2 below.

### Comparative Examples 2 to 5

Organic EL devices were produced in the same manner as in Example 2 except that the second hole transporting layer material was changed to Comparative Compounds 2 to 5, respectively, as shown in Table 2 below.

### Evaluation of Organic EL Device

### Lifetime Measurement

The 95% lifetime (LT95) of the resultant organic EL devices was measured in the same manner as in Example 1. The results are shown in Table 2.

TABLE 2

| | Second Hole Transporting | |
|---|---|---|
| | Layer Material | LT95 [h] |
| Example 2 | Compound 2 | 95 |
| Example 3 | Compound 3 | 98 |
| Example 4 | Compound 5 | 92 |
| Example 5 | Compound 10 | 90 |
| Example 6 | Compound 11 | 88 |
| Example 7 | Compound 12 | 94 |
| Comparative Example 2 | Comparative Compound 2 | 77 |
| Comparative Example 3 | Comparative Compound 3 | 69 |
| Comparative Example 4 | Comparative Compound 4 | 76 |
| Comparative Example 5 | Comparative Compound 5 | 72 |

As obvious from the results in Table 2, it is known that the monoamines (Compounds 2, 3, 5 and 10 to 12 in Examples 2 to 7) satisfying the definition in the present invention exhibit an extremely improved value of LT95, as compared with the monoamines (Comparative Compounds 2 to 5 in Comparative Examples 2 to 5) not satisfying the definition in the present invention.

### Example 8

A glass substrate of 25 mm×75 mm×1.1 mm provided with an ITO transparent electrode (anode) (manufactured by GEOMATEC Co., Ltd.) was ultrasonically cleaned in isopropyl alcohol for 5 minutes and then subjected to UV ozone cleaning for 30 minutes. The film thickness of the ITO was 130 nm.

The cleaned glass substrate provided with the transparent electrode was mounted on a substrate holder of a vacuum vapor deposition apparatus, and firstly, Compound HT-3 and Compound HA were vapor co-deposited on the surface having the transparent electrode formed thereon, so as to cover the transparent electrode, resulting in a hole injecting layer with a film thickness of 10 nm. The mass ratio of Compound HT-3 to Compound HA (HT-3/HA) was 97/3.

Subsequently, on this hole injecting layer, Compound HT-3 was vapor deposited to form a first hole transporting layer with a film thickness of 80 nm.

Subsequently, on this first hole transporting layer, Compound 4 was vapor deposited to form a second hole transporting layer with a film thickness of 10 nm.

Subsequently, on this second hole transporting layer, Compound BH-2 (host material) and Compound BD-1 (dopant material) were vapor co-deposited to form a light emitting layer with a film thickness of 25 nm. The mass ratio of Compound BH-2 to Compound BD-1 (BH-2/BD-1) was 96/4.

Subsequently, on this light emitting layer, Compound ET-3 was vapor deposited to form a first electron transporting layer with a film thickness of 5 nm.

Subsequently, on this first electron transporting layer, Compound ET-4 and Liq were vapor co-deposited to form a second electron transporting layer with a film thickness of 20 nm.

The mass ratio of Compound ET-4 to Liq (ET-4/Liq) was 50/50.

Subsequently, on this second electron transporting layer, LiF was vapor deposited to form an electron injecting electrode with a film thickness of 1 nm.

Then, on this electron injecting electrode, metal Al was vapor deposited to form a metal cathode with a film thickness of 50 nm.

The layer configuration of the organic EL device of Example 8 thus obtained is shown as follows:

ITO(130)/HT-3:HA=97:3(10)/HT-3(80)/Compound 4(10)/BH-2:BD-1=96:4 (25)/ET-3(5)/ET-4:Liq=50:50(20)/LiF(1)/Al(50)

### Examples 9 to 17

Organic EL devices were produced in the same manner as in Example 8 except that the second hole transporting layer material was changed to Compounds 6, 7, 9, 17, 18 to 19, and 22 to 24, respectively, as shown in Table 3 below.

### Comparative Examples 6 and 7

Organic EL devices were produced in the same manner as in Example 8 except that the second hole transporting layer material was changed to Comparative Compounds 6 and 7, respectively, as shown in Table 3 below.

### Evaluation of Organic EL Device

### Lifetime Measurement

The 95% lifetime (LT95) of the resultant organic EL devices was measured in the same manner as in Example 1. The results are shown in Table 3.

TABLE 3

| | Second Hole Transporting | |
|---|---|---|
| | Layer Material | LT95 [h] |
| Example 8 | Compound 4 | 113 |
| Example 9 | Compound 6 | 107 |
| Example 10 | Compound 7 | 104 |
| Example 11 | Compound 9 | 125 |
| Example 12 | Compound 17 | 105 |
| Example 13 | Compound 18 | 100 |
| Example 14 | Compound 19 | 99 |
| Example 15 | Compound 22 | 98 |
| Example 16 | Compound 23 | 96 |
| Example 17 | Compound 24 | 102 |
| Comparative Example 6 | Comparative Example 6 | 84 |
| Comparative Example 7 | Comparative Example 7 | 88 |

As obvious from the results in Table 3, it is known that the monoamines (Compounds 4, 6, 7, 9, 17 to 19, and 22 to 24 in Examples 8 to 17) satisfying the definition in the present invention exhibit an extremely improved value of LT95, as compared with the monoamines (Comparative Compounds 6 and 7 in Comparative Examples 6 and 7) not satisfying the definition in the present invention.

### Example 18

A glass substrate of 25 mm×75 mm×1.1 mm provided with an ITO transparent electrode (anode) (manufactured by GEOMATEC Co., Ltd.) was ultrasonically cleaned in isopropyl alcohol for 5 minutes and then subjected to UV ozone cleaning for 30 minutes. The film thickness of the ITO was 130 nm.

The cleaned glass substrate provided with the transparent electrode was mounted on a substrate holder of a vacuum vapor deposition apparatus, and firstly, Compound HT-4 and Compound HA were vapor co-deposited on the surface having the transparent electrode formed thereon, so as to cover the transparent electrode, resulting in a hole injecting layer with a film thickness of 10 nm. The mass ratio of Compound HT-4 to Compound HA (HT-4/HA) was 97/3.

Subsequently, on this hole injecting layer, Compound HT-4 was vapor deposited to form a first hole transporting layer with a film thickness of 75 nm.

Subsequently, on this first hole transporting layer, Compound 6 was vapor deposited to form a second hole transporting layer with a film thickness of 7.5 nm.

Subsequently, on this second hole transporting layer, Compound BH-3 (host material), Compound BH-4 (host material) and Compound BD-2 (dopant material) were vapor co-deposited to form a light emitting layer with a film thickness of 20 nm. The mass ratio of Compound BH-3 to Compound BH-4 to Compound BD-2 (BH-23/BH-4/BD-2) was 60/40/2.

Subsequently, on this light emitting layer, Compound ET-5 was vapor deposited to form a first electron transporting layer with a film thickness of 3 nm.

Subsequently, on this first electron transporting layer, Compound ET-2 and Liq were vapor co-deposited to form a second electron transporting layer with a film thickness of 30 nm. The mass ratio of Compound ET-2 to Liq (ET-2/Liq) was 50/50.

Subsequently, on this second electron transporting layer, LiF and YB was vapor co-deposited to form an electron injecting electrode with a film thickness of 1 nm. The mass ratio of LiF to Yb (LiF/Yb) was 50/50.

Then, on this electron injecting electrode, metal Al was vapor deposited to form a metal cathode with a film thickness of 50 nm.

The layer configuration of the organic EL device of Example 18 thus obtained is shown as follows:

ITO(130)/HT-4:HA=97:3(10)/HT-4(75)/Compound 6(7.5)/BH-3:BH-4:BD-2=60:40:2(20)/ET-5(3)/ET-2:Liq=50:50(30)/LiF:Yb=50:50(1)/Al(50)

### Examples 19 to 22

Organic EL devices were produced in the same manner as in Example 18 except that the second hole transporting layer material was changed to Compounds 6, 9, 18, 20, and 21, respectively, as shown in Table 4 below.

### Comparative Example 8

An organic EL device was produced in the same manner as in Example 18 except that the second hole transporting layer material was changed to Comparative Compound 8, as shown in Table 4 below.

### Evaluation of Organic EL Device

### Lifetime Measurement

The 95% lifetime (LT95) of the resultant organic EL devices was measured in the same manner as in Example 1. The results are shown in Table 4.

TABLE 4

| | Second Hole Transporting | |
|---|---|---|
| | Layer Material | LT95 [h] |
| Example 18 | Compound 6 | 108 |
| Example 19 | Compound 9 | 129 |
| Example 20 | Compound 18 | 101 |
| Example 21 | Compound 20 | 116 |
| Example 22 | Compound 21 | 106 |
| Comparative Example 8 | Comparative Compound 8 | 90 |

As obvious from the results in Table 4, it is known that the monoamines (Compounds 6, 9, 18, 20, 21 in Examples 18 to 22) satisfying the definition in the present invention exhibit an extremely improved value of LT95, as compared with the monoamine (Comparative Compound 8 in Comparative Example 8) not satisfying the definition in the present invention.

### Compounds 1 to 17 Synthesized in Synthesis Examples 1 to 17

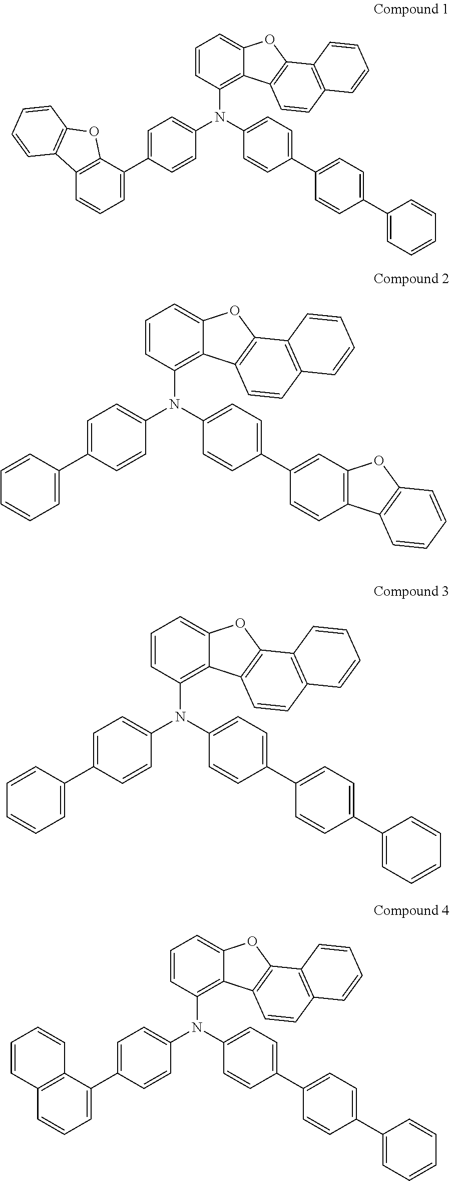 [SMILES: c1ccc(-c2ccc(-c3ccc(N(c4ccc(-c5cccc6c5oc5ccccc56)cc4)c4cccc5oc6c7ccccc7ccc6c45)cc3)cc2)cc1 ; c1ccc(-c2ccc(N(c3ccc(-c4ccc5c(c4)oc4ccccc45)cc3)c3cccc4oc5c6ccccc6ccc5c34)cc2)cc1 ; c1ccc(-c2ccc(-c3ccc(N(c4ccc(-c5ccccc5)cc4)c4cccc5oc6c7ccccc7ccc6c45)cc3)cc2)cc1 ; c1ccc(-c2ccc(-c3ccc(N(c4ccc(-c5cccc6ccccc56)cc4)c4cccc5oc6c7ccccc7ccc6c45)cc3)cc2)cc1]

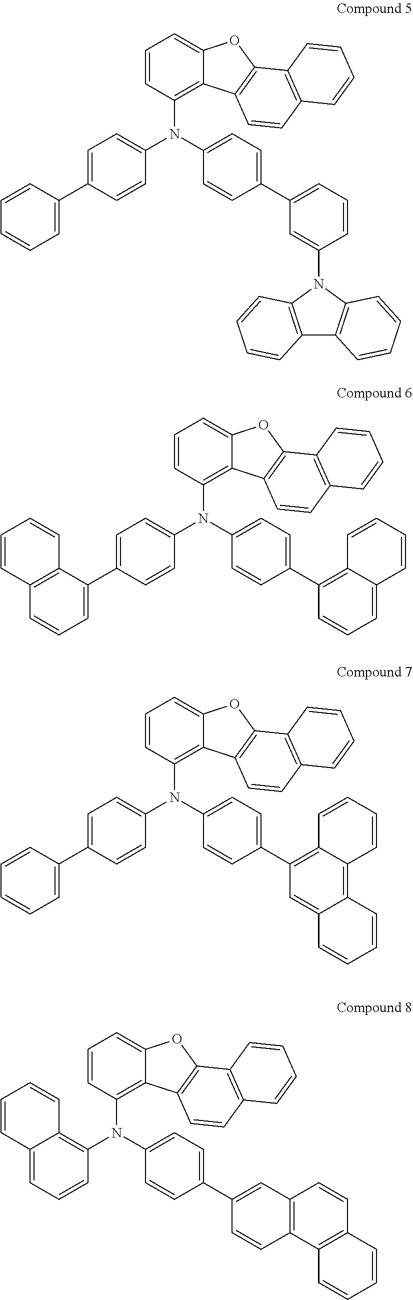 [SMILES: c1ccc(-c2ccc(N(c3ccc(-c4cccc(-n5c6ccccc6c6ccccc65)c4)cc3)c3cccc4oc5c6ccccc6ccc5c34)cc2)cc1 ; c1ccc2c(-c3ccc(N(c4ccc(-c5cccc6ccccc56)cc4)c4cccc5oc6c7ccccc7ccc6c45)cc3)cccc2c1 ; c1ccc(-c2ccc(N(c3ccc(-c4cc5ccccc5c5ccccc45)cc3)c3cccc4oc5c6ccccc6ccc5c34)cc2)cc1 ; c1ccc2c(N(c3ccc(-c4ccc5c(ccc6ccccc65)c4)cc3)c3cccc4oc5c6ccccc6ccc5c34)cccc2c1]

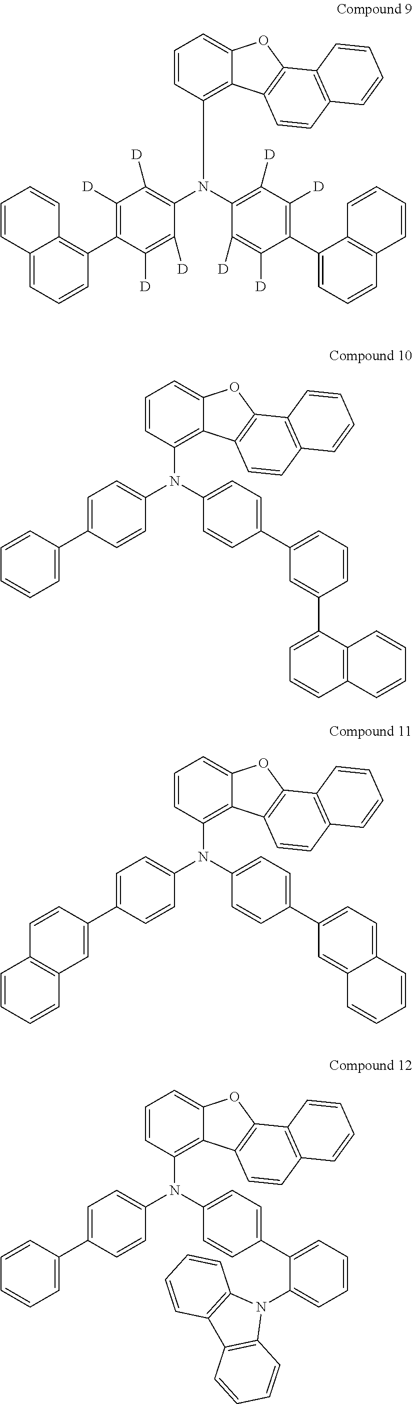 [SMILES: [2H]c1c([2H])c(N(c2c([2H])c([2H])c(-c3cccc4ccccc34)c([2H])c2[2H])c2cccc3oc4c5ccccc5ccc4c23)c([2H])c([2H])c1-c1cccc2ccccc12 ; c1ccc(-c2ccc(N(c3ccc(-c4cccc(-c5cccc6ccccc56)c4)cc3)c3cccc4oc5c6ccccc6ccc5c34)cc2)cc1 ; c1ccc2cc(-c3ccc(N(c4ccc(-c5ccc6ccccc6c5)cc4)c4cccc5oc6c7ccccc7ccc6c45)cc3)ccc2c1 ; c1ccc(-c2ccc(N(c3ccc(-c4ccccc4-n4c5ccccc5c5ccccc54)cc3)c3cccc4oc5c6ccccc6ccc5c34)cc2)cc1]

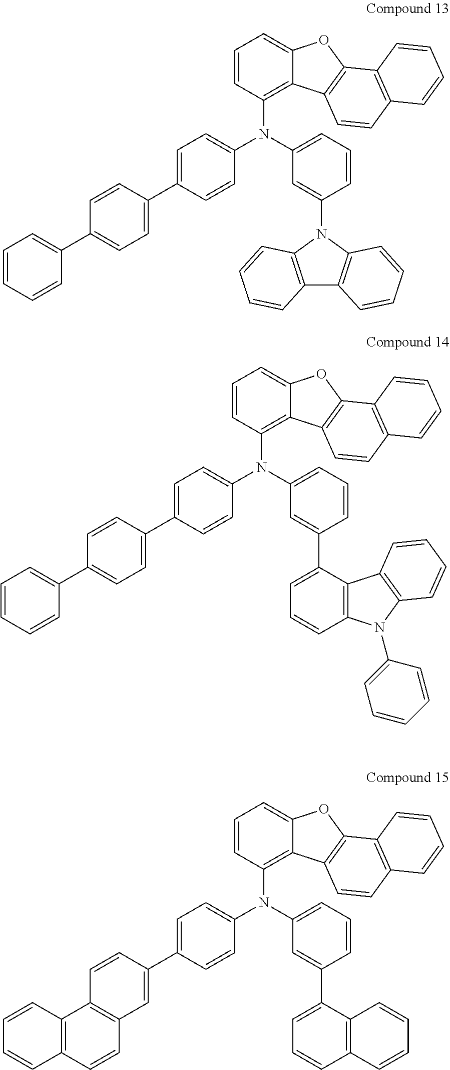 [SMILES: c1ccc(-c2ccc(-c3ccc(N(c4cccc(-n5c6ccccc6c6ccccc65)c4)c4cccc5oc6c7ccccc7ccc6c45)cc3)cc2)cc1 ; c1ccc(-c2ccc(-c3ccc(N(c4cccc(-c5cccc6c5c5ccccc5n6-c5ccccc5)c4)c4cccc5oc6c7ccccc7ccc6c45)cc3)cc2)cc1 ; c1cc(-c2cccc3ccccc23)cc(N(c2ccc(-c3ccc4c(ccc5ccccc54)c3)cc2)c2cccc3oc4c5ccccc5ccc4c23)c1]

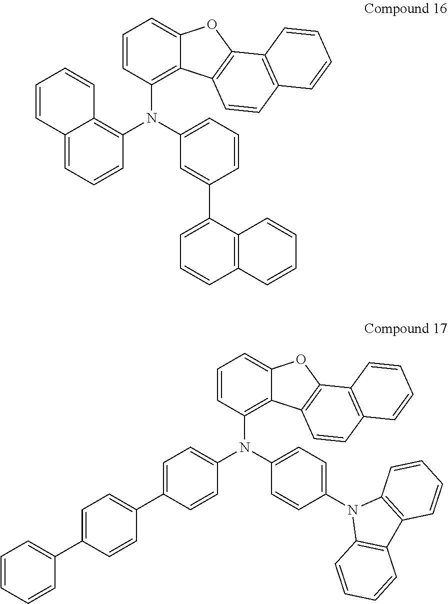 [SMILES: c1cc(-c2cccc3ccccc23)cc(N(c2cccc3ccccc23)c2cccc3oc4c5ccccc5ccc4c23)c1 ; c1ccc(-c2ccc(-c3ccc(N(c4ccc(-n5c6ccccc6c6ccccc65)cc4)c4cccc5oc6c7ccccc7ccc6c45)cc3)cc2)cc1]

### Compounds 18 to 24 Synthesized in Synthesis Examples 18 to 24

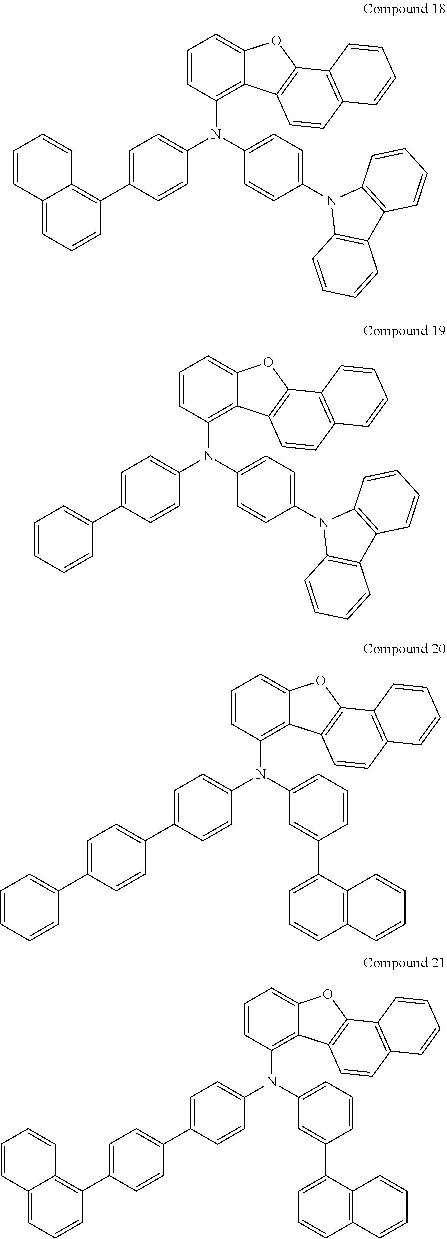 [SMILES: c1ccc2c(-c3ccc(N(c4ccc(-n5c6ccccc6c6ccccc65)cc4)c4cccc5oc6c7ccccc7ccc6c45)cc3)cccc2c1 ; c1ccc(-c2ccc(N(c3ccc(-n4c5ccccc5c5ccccc54)cc3)c3cccc4oc5c6ccccc6ccc5c34)cc2)cc1 ; c1ccc(-c2ccc(-c3ccc(N(c4cccc(-c5cccc6ccccc56)c4)c4cccc5oc6c7ccccc7ccc6c45)cc3)cc2)cc1 ; c1cc(-c2cccc3ccccc23)cc(N(c2ccc(-c3ccc(-c4cccc5ccccc45)cc3)cc2)c2cccc3oc4c5ccccc5ccc4c23)c1]

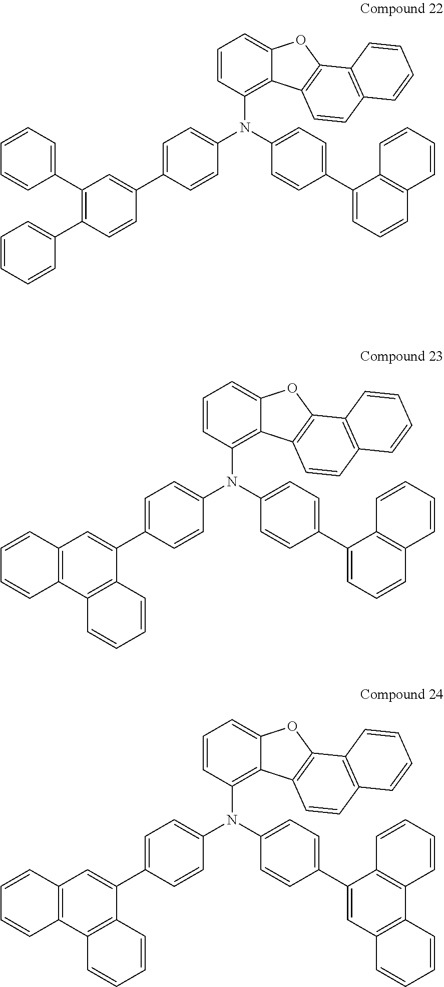 [SMILES: c1ccc(-c2ccc(-c3ccc(N(c4ccc(-c5cccc6ccccc56)cc4)c4cccc5oc6c7ccccc7ccc6c45)cc3)cc2-c2ccccc2)cc1 ; c1ccc2c(-c3ccc(N(c4ccc(-c5cc6ccccc6c6ccccc56)cc4)c4cccc5oc6c7ccccc7ccc6c45)cc3)cccc2c1 ; c1ccc2c(c1)cc(-c1ccc(N(c3ccc(-c4cc5ccccc5c5ccccc45)cc3)c3cccc4oc5c6ccccc6ccc5c34)cc1)c1ccccc12]

### <Synthesis of Compound>

### Intermediate Synthesis Example 1: Synthesis of Intermediate A

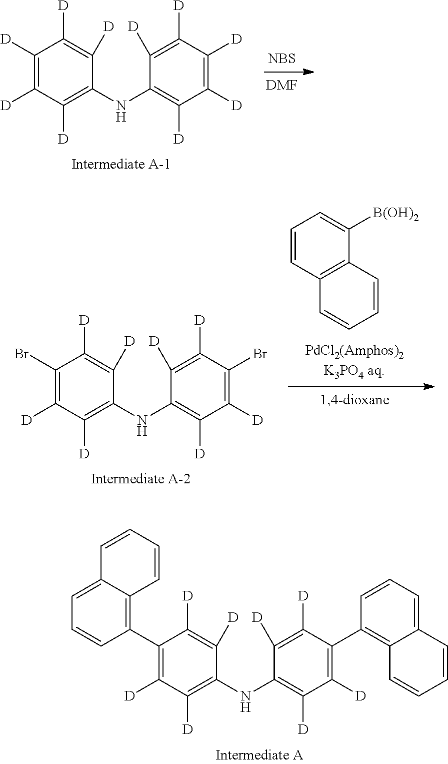

In an argon atmosphere, Intermediate A-1 (2.9 g, 16.18 mmol) and DMF (55 ml) were mixed, and at 0° C., N-bromosuccinimide (5.76 g, 32.4 mmol) was added. Water and ethyl acetate were added for extraction, and the resultant organic layer was evaporated under reduced pressure to give Intermediate A-2. Not purified, Intermediate A-2 was subjected to the next reaction.

In an argon atmosphere, Intermediate A-2 (6.41 g, 19.12 mmol), 1-naphthylboronic acid (8.22 g, 47.8 mmol), bis(di-t-butyl(4-dimethylaminophenyl)phosphine)dichloropalladium(II) (406 mg, 0.574 mmol), and 1,4-dioxane (100 ml) were mixed, and an aqueous solution of potassium phosphate was added. At 110° C., this was heated and stirred for 7 hours, then left cooled, and the mixture was filtered and purified by column chromatography and recrystallization to give Intermediate A (4.9 g). The yield was 71% (2 steps).

### Intermediate Synthesis Example 2: Synthesis of Intermediate B

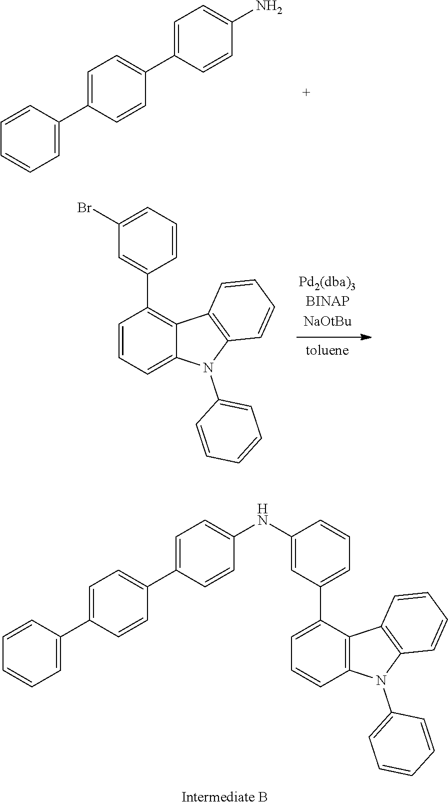 [SMILES: Nc1ccc(-c2ccc(-c3ccccc3)cc2)cc1 ; Brc1cccc(-c2cccc3c2c2ccccc2n3-c2ccccc2)c1 ; c1ccc(-c2ccc(-c3ccc(Nc4cccc(-c5cccc6c5c5ccccc5n6-c5ccccc5)c4)cc3)cc2)cc1]

In an argon atmosphere, a mixture of [1,1′:4′,1″-terphenyl]-4-amine (4.91 g, 20.0 mmol), 4-(3-bromophenyl)-9-phenyl-9H-carbazole (7.97 g, 20.0 mmol), tris(dibenzylideneacetone)dipalladium(0) (0.366 g, 0.400 mmol), BINAP (0.498 g, 0.800 mmol), sodium t-butoxide (2.11 g, 22.0 mmol) and toluene (100 mL) was stirred at 100° C. for 7 hours. The reaction liquid was cooled down to room temperature, and then concentrated under reduced pressure. The resultant residue was purified by silica gel column chromatography and recrystallization to give 6.41 g of a white solid of Intermediate B. The yield was 79%.

### Intermediate Synthesis Example 3: Synthesis of Intermediate C

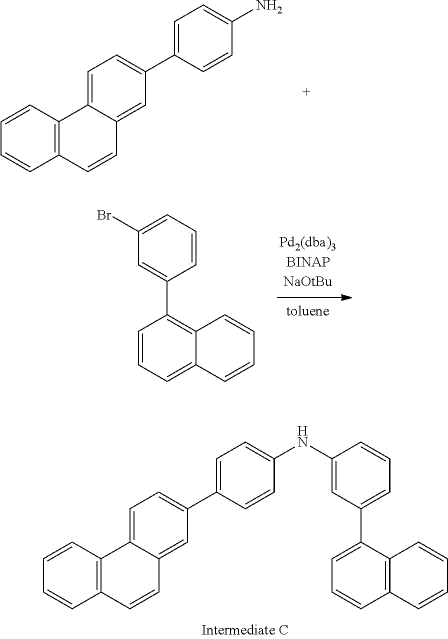 [SMILES: Nc1ccc(-c2ccc3c(ccc4ccccc43)c2)cc1 ; Brc1cccc(-c2cccc3ccccc23)c1 ; c1cc(Nc2ccc(-c3ccc4c(ccc5ccccc54)c3)cc2)cc(-c2cccc3ccccc23)c1]

In the same manner as in synthesis of Intermediate B except that 4-(2-phenanthryl)benzenamine was used in place of [1,1′:4′,1″-terphenyl]-4-amine and 1-(3-bromophenyl)naphthalene was used in place of 4-(3-bromophenyl)-9-phenyl-9H-carbazole, a white solid of Intermediate C was produced.

### Intermediate Synthesis Example 4: Synthesis of Intermediate D

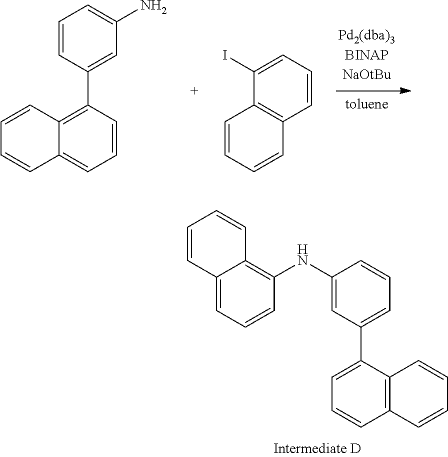 [SMILES: Nc1cccc(-c2cccc3ccccc23)c1 ; Ic1cccc2ccccc12 ; c1cc(Nc2cccc3ccccc23)cc(-c2cccc3ccccc23)c1]

In the same manner as in synthesis of Intermediate B except that 3-(1-naphthalenyl)benzenamine was used in place of [1,1′:4′,1″-terphenyl]-4-amine and 1-iodonaphthalene was used in place of 4-(3-bromophenyl)-9-phenyl-9H-carbazole, a white solid of Intermediate D was produced.

### Intermediate Synthesis Example 5: Synthesis of Intermediate E

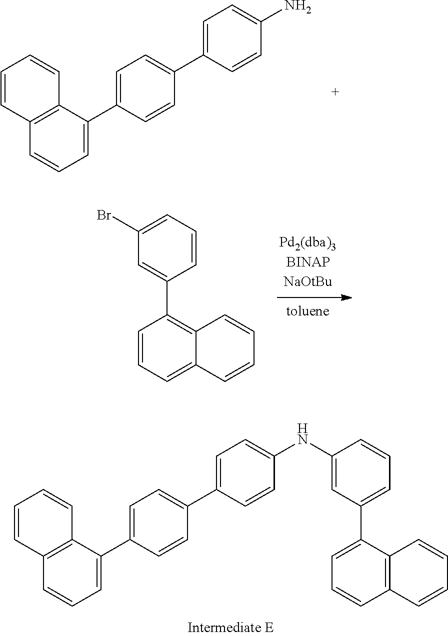 [SMILES: Nc1ccc(-c2ccc(-c3cccc4ccccc34)cc2)cc1 ; Brc1cccc(-c2cccc3ccccc23)c1 ; c1cc(Nc2ccc(-c3ccc(-c4cccc5ccccc45)cc3)cc2)cc(-c2cccc3ccccc23)c1]

In the same manner as in synthesis of Intermediate B except that 4′-(1-napthalenyl)[1,1′-biphenyl]-4-amine was used in place of [1,1′:4′,1″-terphenyl]-4-amine and 1-(3-bromophenyl)naphthalene was used in place of 4-(3-bromophenyl)-9-phenyl-9H-carbazole, a white solid of Intermediate E was produced.

### Intermediate Synthesis Example 6: Synthesis of Intermediate F

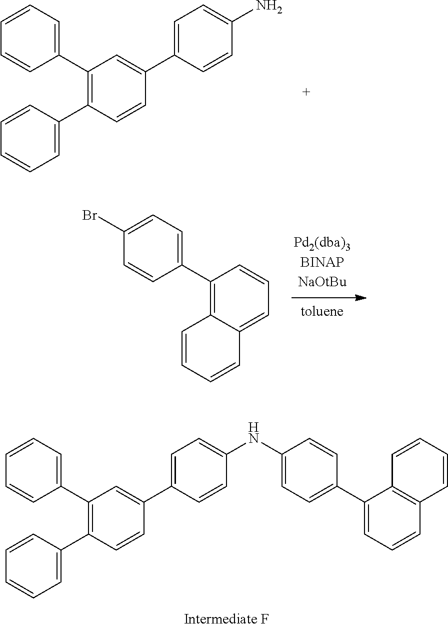 [SMILES: Nc1ccc(-c2ccc(-c3ccccc3)c(-c3ccccc3)c2)cc1 ; Brc1ccc(-c2cccc3ccccc23)cc1 ; c1ccc(-c2ccc(-c3ccc(Nc4ccc(-c5cccc6ccccc56)cc4)cc3)cc2-c2ccccc2)cc1]

In the same manner as in synthesis of Intermediate B except that 4′-phenyl-[1,1′:3′,1″-terphenyl]-4-amine was used in place of [1,1′:4′,1″-terphenyl]-4-amine and 1-(4-bromophenyl)naphthalene was used in place of 4-(3-bromophenyl)-9-phenyl-9H-carbazole, a white solid of Intermediate F was produced.

### Synthesis Example 1: Synthesis of Compound 1

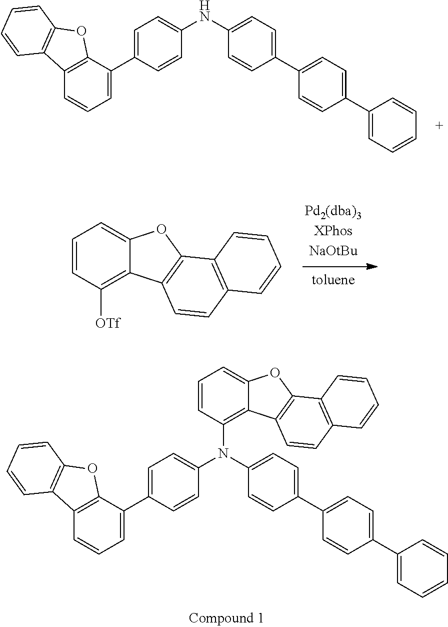 [SMILES: c1ccc(-c2ccc(-c3ccc(Nc4ccc(-c5cccc6c5oc5ccccc56)cc4)cc3)cc2)cc1 ; O=S(=O)(Oc1cccc2oc3c4ccccc4ccc3c12)C(F)(F)F ; c1ccc(-c2ccc(-c3ccc(N(c4ccc(-c5cccc6c5oc5ccccc56)cc4)c4cccc5oc6c7ccccc7ccc6c45)cc3)cc2)cc1]

In an argon atmosphere, a mixture of N-[4-(dibenzo[b,d]furan-4-yl)phenyl][1,1′:4′,1″-terphenyl]-4-amine (4.88 g, 10.0 mmol), naphtho[1.2-b]benzofuran-7-yl trifluoromethanesulfonate (4.03 g, 11.0 mmol), tris(dibenzylideneacetone)dipalladium(0) (0.183 g, 0.200 mmol, XPhos (0.364 g, 0.764 mmol), sodium t-butoxide (1.35 g, 14.0 mmol), and toluene (100 mL) was stirred at 100° C. for 7 hours. The reaction liquid was cooled down to room temperature, and then concentrated under reduced pressure. The resultant residue was purified by silica gel column chromatography and recrystallization to give 6.41 g of a white solid. The yield was 91%.

As a result of mass spectrometry, the product was Compound 1 having m/e=704 relative to the molecular weight of 703.84.

### Synthesis Example 2: Synthesis of Compound 2

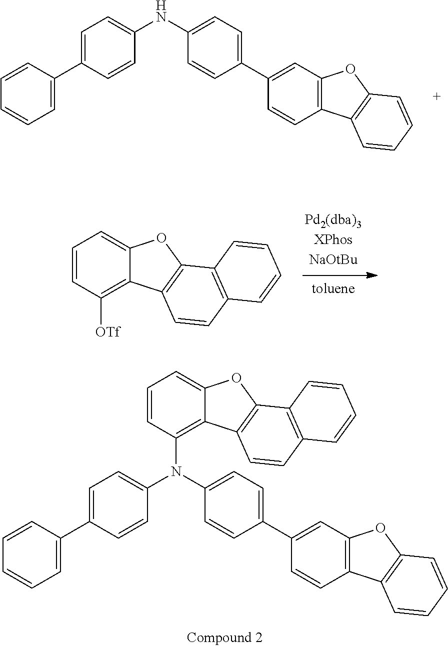 [SMILES: c1ccc(-c2ccc(Nc3ccc(-c4ccc5c(c4)oc4ccccc45)cc3)cc2)cc1 ; O=S(=O)(Oc1cccc2oc3c4ccccc4ccc3c12)C(F)(F)F ; c1ccc(-c2ccc(N(c3ccc(-c4ccc5c(c4)oc4ccccc45)cc3)c3cccc4oc5c6ccccc6ccc5c34)cc2)cc1]

In the same manner as in Synthesis Example 1 except that N-[4-(dibenzo[b,d]furan-3-yl)phenyl][1,1′-biphenyl]-4-amine was used in place of N-[4-(dibenzo[b,d]furan-4-yl)phenyl][1,1′:4′,1″-terphenyl]-4-amine, a white solid was produced.

As a result of mass spectrometry, the product was Compound 2 having m/e=628 relative to the molecular weight of 627.74.

### Synthesis Example 3: Synthesis of Compound 3

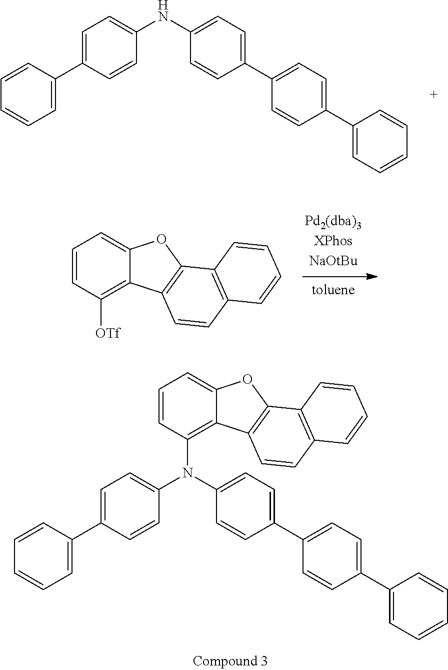 [SMILES: c1ccc(-c2ccc(Nc3ccc(-c4ccc(-c5ccccc5)cc4)cc3)cc2)cc1 ; O=S(=O)(Oc1cccc2oc3c4ccccc4ccc3c12)C(F)(F)F ; c1ccc(-c2ccc(-c3ccc(N(c4ccc(-c5ccccc5)cc4)c4cccc5oc6c7ccccc7ccc6c45)cc3)cc2)cc1]

In the same manner as in Synthesis Example 1 except that N-[1,1′-biphenyl]-4-yl-[1,1′:4′,1″-terphenyl]-4-amine was used in place of N-[4-(dibenzo[b,d]furan-4-yl)phenyl][1,1′:4′,1″-terphenyl]-4-amine, a white solid was produced.

As a result of mass spectrometry, the product was Compound 3 having m/e=614 relative to the molecular weight of 613.76.

### Synthesis Example 4: Synthesis of Compound 4

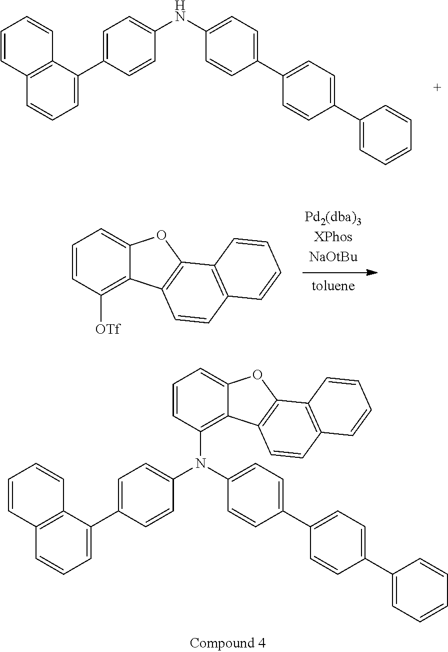 [SMILES: c1ccc(-c2ccc(-c3ccc(Nc4ccc(-c5cccc6ccccc56)cc4)cc3)cc2)cc1 ; O=S(=O)(Oc1cccc2oc3c4ccccc4ccc3c12)C(F)(F)F ; c1ccc(-c2ccc(-c3ccc(N(c4ccc(-c5cccc6ccccc56)cc4)c4cccc5oc6c7ccccc7ccc6c45)cc3)cc2)cc1]

In the same manner as in Synthesis Example 1 except that N-[4-(naphthalen-1-yl)phenyl][1,1′:4′,1″-terphenyl]-4-amine was used in place of N-[4-(dibenzo[b,d]furan-4-yl)phenyl][1,1′:4′,1″-terphenyl]-4-amine, a white solid was produced.

As a result of mass spectrometry, the product was Compound 4 having m/e=664 relative to the molecular weight of 663.82.

### Synthesis Example 5: Synthesis of Compound 5

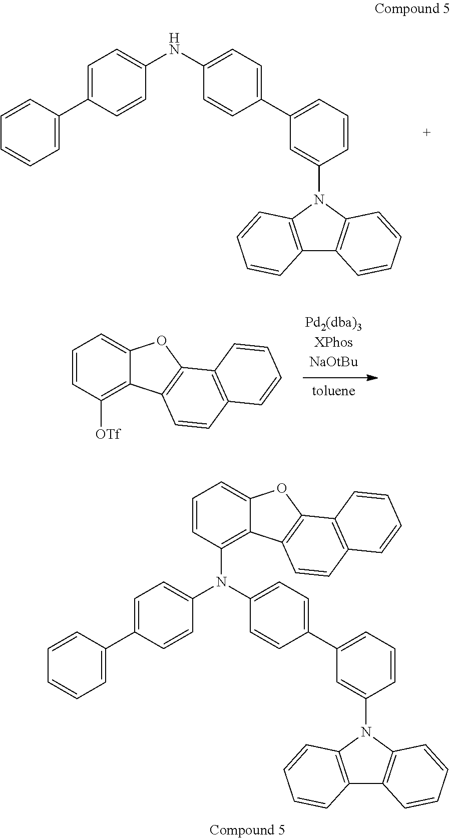 [SMILES: c1ccc(-c2ccc(Nc3ccc(-c4cccc(-n5c6ccccc6c6ccccc65)c4)cc3)cc2)cc1 ; O=S(=O)(Oc1cccc2oc3c4ccccc4ccc3c12)C(F)(F)F ; c1ccc(-c2ccc(N(c3ccc(-c4cccc(-n5c6ccccc6c6ccccc65)c4)cc3)c3cccc4oc5c6ccccc6ccc5c34)cc2)cc1]

In the same manner as in Synthesis Example 1 except that N-[3′-(9H-carbazol-9-yl)[1,1′-biphenyl]-4-yl][1,1′-biphenyl]-4-amine was used in place of N-[4-(dibenzo[b,d]furan-4-yl)phenyl][1,1′:4′,1″-terphenyl]-4-amine, a white solid was produced.

As a result of mass spectrometry, the product was Compound 5 having m/e=703 relative to the molecular weight of 702.86.

### Synthesis Example 6: Synthesis of Compound 6

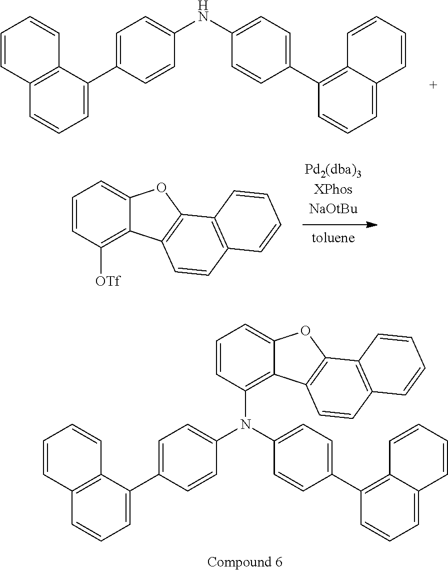 [SMILES: c1ccc2c(-c3ccc(Nc4ccc(-c5cccc6ccccc56)cc4)cc3)cccc2c1 ; O=S(=O)(Oc1cccc2oc3c4ccccc4ccc3c12)C(F)(F)F ; c1ccc2c(-c3ccc(N(c4ccc(-c5cccc6ccccc56)cc4)c4cccc5oc6c7ccccc7ccc6c45)cc3)cccc2c1]

In the same manner as in Synthesis Example 1 except that 4-(naphthalen-1-yl)-N-[4-naphthalen-1-yl)phenyl]benzenamine was used in place of N-[4-(dibenzo[b,d]furan-4-yl)phenyl][1,1′:4′,1″-terphenyl]-4-amine, a white solid was produced.

As a result of mass spectrometry, the product was Compound 6 having m/e=638 relative to the molecular weight of 637.78.

### Synthesis Example 7: Synthesis of Compound 7

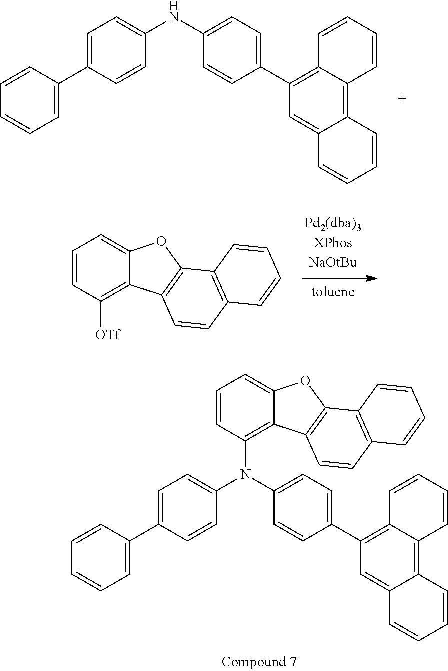 [SMILES: c1ccc(-c2ccc(Nc3ccc(-c4cc5ccccc5c5ccccc45)cc3)cc2)cc1 ; O=S(=O)(Oc1cccc2oc3c4ccccc4ccc3c12)C(F)(F)F ; c1ccc(-c2ccc(N(c3ccc(-c4cc5ccccc5c5ccccc45)cc3)c3cccc4oc5c6ccccc6ccc5c34)cc2)cc1]

In the same manner as in Synthesis Example 1 except that N-[4-(473henanthrene-9-yl)phenyl][1,1′-biphenyl]-4-amine was used in place of N-[4-(dibenzo[b,d]furan-4-yl)phenyl][1,1′:4′,1″-terphenyl]-4-amine, a white solid was produced.

As a result of mass spectrometry, the product was Compound 7 having m/e=638 relative to the molecular weight of 637.78.

### Synthesis Example 8: Synthesis of Compound 8

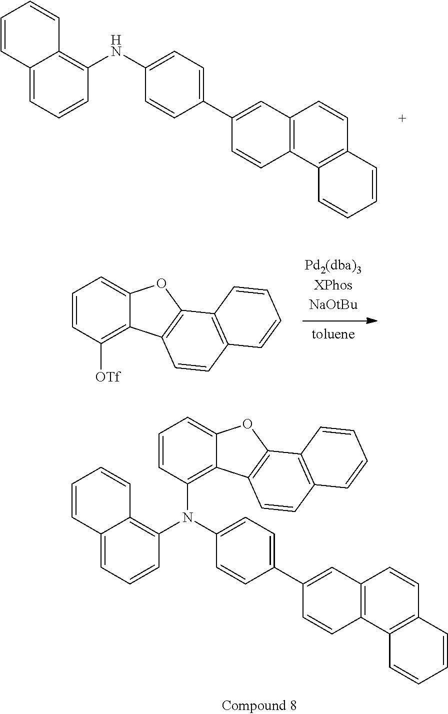 [SMILES: c1ccc2c(Nc3ccc(-c4ccc5c(ccc6ccccc65)c4)cc3)cccc2c1 ; O=S(=O)(Oc1cccc2oc3c4ccccc4ccc3c12)C(F)(F)F ; c1ccc2c(N(c3ccc(-c4ccc5c(ccc6ccccc65)c4)cc3)c3cccc4oc5c6ccccc6ccc5c34)cccc2c1]

In the same manner as in Synthesis Example 1 except that N-[4-(phenanthren-2-yl)phenyl]naphthalene-1-amine was used in place of N-[4-(dibenzo[b,d]furan-4-yl)phenyl][1,1′:4′,1″-terphenyl]-4-amine, a white solid was produced.

As a result of mass spectrometry, the product was Compound 8 having m/e=612 relative to the molecular weight of 611.74.

### Synthesis Example 9: Synthesis of Compound 9

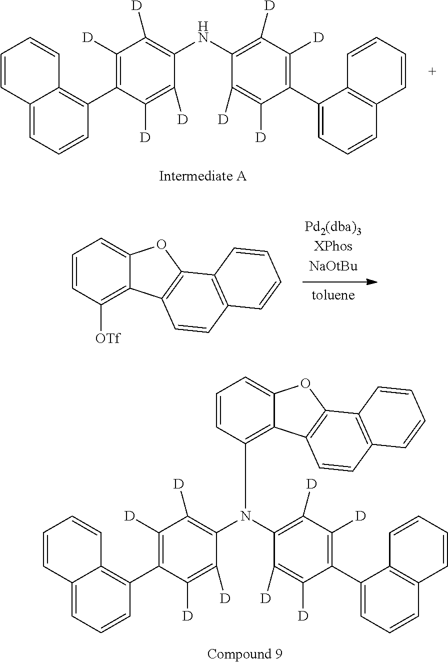 [SMILES: [2H]c1c([2H])c(-c2cccc3ccccc23)c([2H])c([2H])c1Nc1c([2H])c([2H])c(-c2cccc3ccccc23)c([2H])c1[2H] ; O=S(=O)(Oc1cccc2oc3c4ccccc4ccc3c12)C(F)(F)F ; [2H]c1c([2H])c(N(c2c([2H])c([2H])c(-c3cccc4ccccc34)c([2H])c2[2H])c2cccc3oc4c5ccccc5ccc4c23)c([2H])c([2H])c1-c1cccc2ccccc12]

In the same manner as in Synthesis Example 1 except that Intermediate A was used in place of N-[4-(dibenzo[b,d]furan-4-yl)phenyl][1,1′:4′,1″-terphenyl]-4-amine, a white solid was produced.

As a result of mass spectrometry, the product was Compound 9 having m/e=646 relative to the molecular weight of 645.83.

### Synthesis Example 10: Synthesis of Compound 10

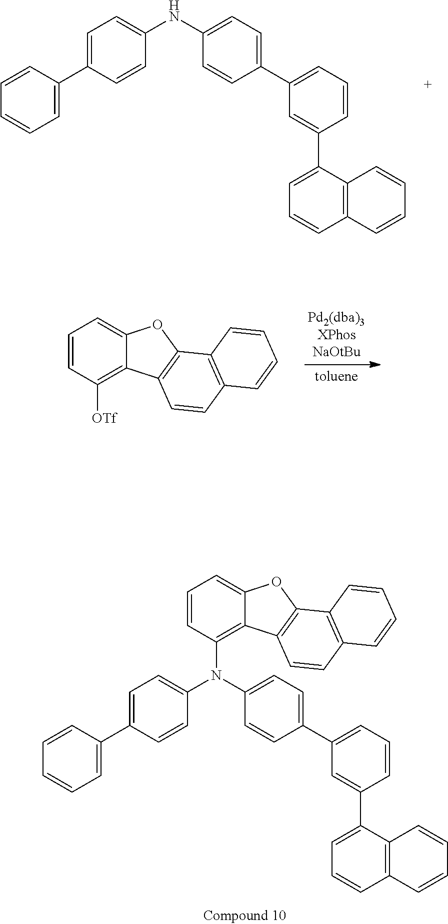 [SMILES: c1ccc(-c2ccc(Nc3ccc(-c4cccc(-c5cccc6ccccc56)c4)cc3)cc2)cc1 ; O=S(=O)(Oc1cccc2oc3c4ccccc4ccc3c12)C(F)(F)F ; c1ccc(-c2ccc(N(c3ccc(-c4cccc(-c5cccc6ccccc56)c4)cc3)c3cccc4oc5c6ccccc6ccc5c34)cc2)cc1]

In the same manner as in Synthesis Example 1 except that N-([1,1′-biphenyl]-4-yl)-3′-(naphthalen-1-yl)[1,1′-biphenyl]4-amine was used in place of N-[4-(dibenzo[b,d]furan-4-yl)phenyl][1,1′:4′,1″-terphenyl]-4-amine, a white solid was produced.

As a result of mass spectrometry, the product was Compound 10 having m/e=664 relative to the molecular weight of 663.82.

### Synthesis Example 11: Synthesis of Compound 11

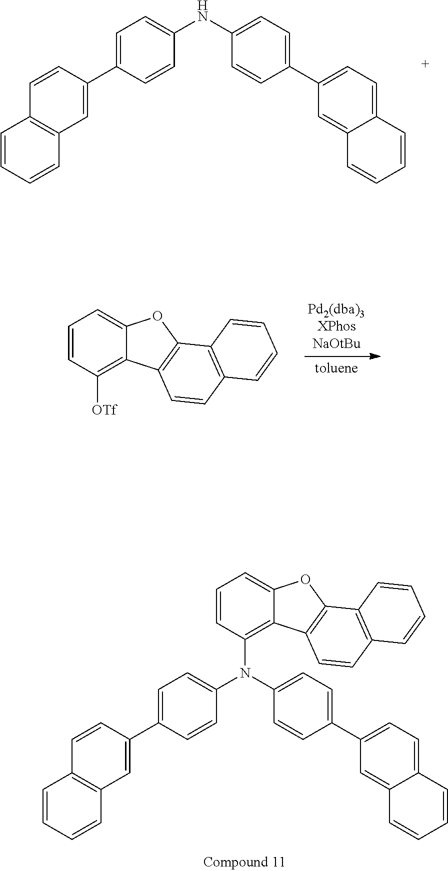 [SMILES: c1ccc2cc(-c3ccc(Nc4ccc(-c5ccc6ccccc6c5)cc4)cc3)ccc2c1 ; O=S(=O)(Oc1cccc2oc3c4ccccc4ccc3c12)C(F)(F)F ; c1ccc2cc(-c3ccc(N(c4ccc(-c5ccc6ccccc6c5)cc4)c4cccc5oc6c7ccccc7ccc6c45)cc3)ccc2c1]

In the same manner as in Synthesis Example 1 except that 4-(naphthalen-2-yl)-N-[4-(naphthalen-2-yl)phenyl]benzenamine was used in place of N-[4-(dibenzo[b,d]furan-4-yl)phenyl][1,1′:4′,1″-terphenyl]-4-amine, a white solid was produced.

As a result of mass spectrometry, the product was Compound 11 having m/e=638 relative to the molecular weight of 637.78.

### Synthesis Example 12: Synthesis of Compound 12

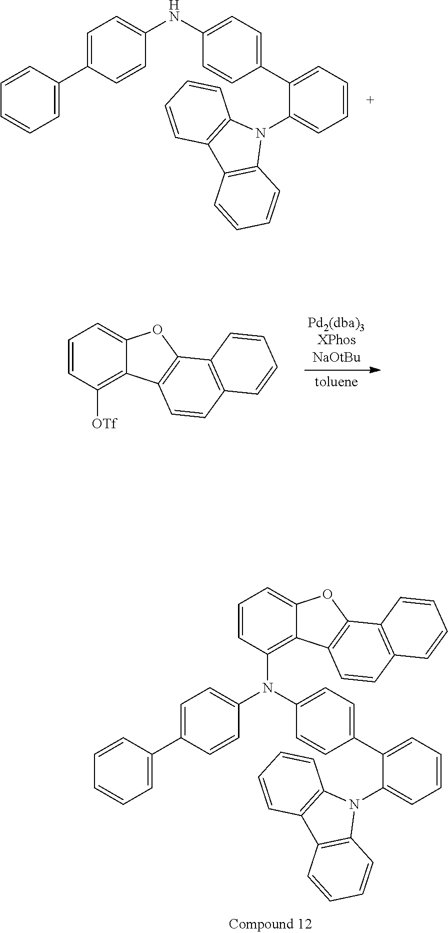 [SMILES: c1ccc(-c2ccc(Nc3ccc(-c4ccccc4-n4c5ccccc5c5ccccc54)cc3)cc2)cc1 ; O=S(=O)(Oc1cccc2oc3c4ccccc4ccc3c12)C(F)(F)F ; c1ccc(-c2ccc(N(c3ccc(-c4ccccc4-n4c5ccccc5c5ccccc54)cc3)c3cccc4oc5c6ccccc6ccc5c34)cc2)cc1]

In the same manner as in Synthesis Example 1 except that N-[2′-(9H-carbazol-9-yl)[1,1′-biphenyl]-4-yl][1,1′-biphenyl]-4-amine was used in place of N-[4-(dibenzo[b,d]furan-4-yl)phenyl][1,1′:4′,1″-terphenyl]-4-amine, a white solid was produced.

As a result of mass spectrometry, the product was Compound 12 having m/e=703 relative to the molecular weight of 702.86.

### Synthesis Example 13: Synthesis of Compound 13

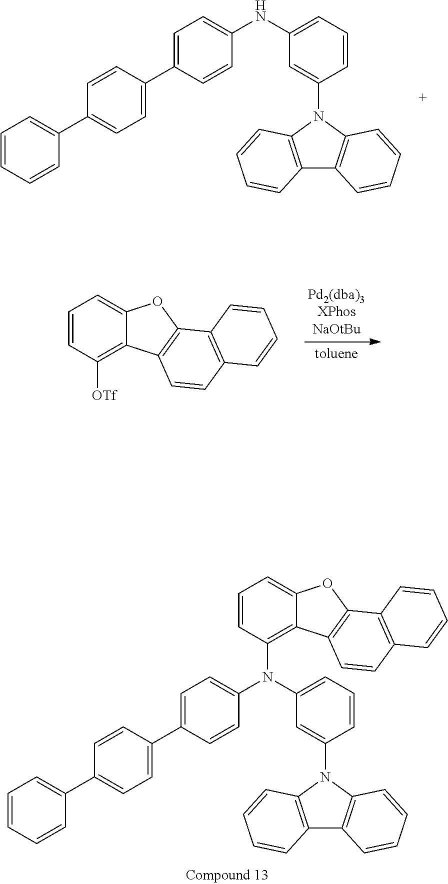 [SMILES: c1ccc(-c2ccc(-c3ccc(Nc4cccc(-n5c6ccccc6c6ccccc65)c4)cc3)cc2)cc1 ; O=S(=O)(Oc1cccc2oc3c4ccccc4ccc3c12)C(F)(F)F ; c1ccc(-c2ccc(-c3ccc(N(c4cccc(-n5c6ccccc6c6ccccc65)c4)c4cccc5oc6c7ccccc7ccc6c45)cc3)cc2)cc1]

In the same manner as in Synthesis Example 1 except that N-[3-(9H-carbazol-9-yl)phenyl][1,1′:4′,1″-terphenyl]-4-amine was used in place of N-[4-(dibenzo[b,d]furan-4-yl)phenyl][1,1′:4′,1″-terphenyl]-4-amine, a white solid was produced.

As a result of mass spectrometry, the product was Compound 13 having m/e=703 relative to the molecular weight of 702.86.

### Synthesis Example 14: Synthesis of Compound 14

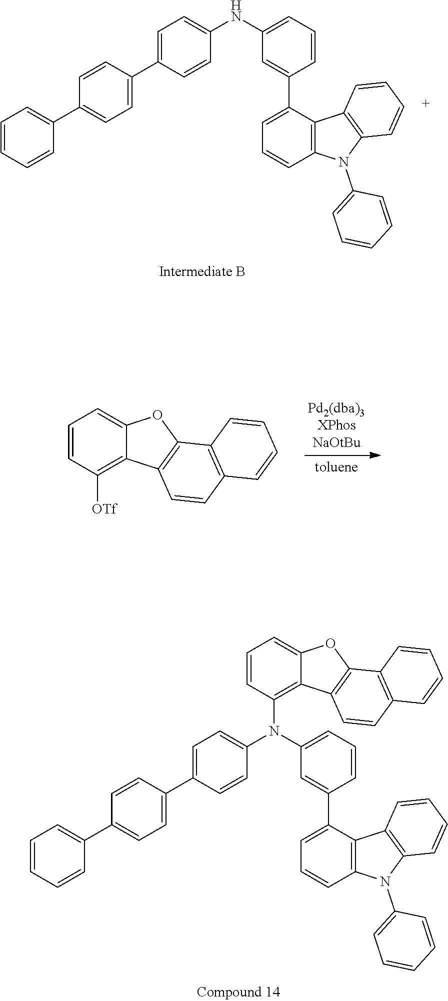 [SMILES: c1ccc(-c2ccc(-c3ccc(Nc4cccc(-c5cccc6c5c5ccccc5n6-c5ccccc5)c4)cc3)cc2)cc1 ; O=S(=O)(Oc1cccc2oc3c4ccccc4ccc3c12)C(F)(F)F ; c1ccc(-c2ccc(-c3ccc(N(c4cccc(-c5cccc6c5c5ccccc5n6-c5ccccc5)c4)c4cccc5oc6c7ccccc7ccc6c45)cc3)cc2)cc1]

In the same manner as in Synthesis Example 1 except that Intermediate B was used in place of N-[4-(dibenzo[b,d]furan-4-yl)phenyl][1,1′:4′,1″-terphenyl]-4-amine, a white solid was produced.

As a result of mass spectrometry, the product was Compound 14 having m/e=779 relative to the molecular weight of 778.96.

### Synthesis Example 15: Synthesis of Compound 15

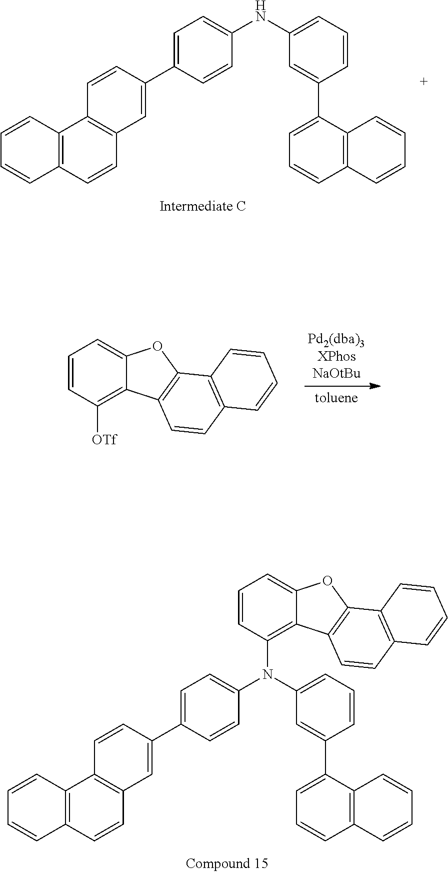 [SMILES: c1cc(Nc2ccc(-c3ccc4c(ccc5ccccc54)c3)cc2)cc(-c2cccc3ccccc23)c1 ; O=S(=O)(Oc1cccc2oc3c4ccccc4ccc3c12)C(F)(F)F ; c1cc(-c2cccc3ccccc23)cc(N(c2ccc(-c3ccc4c(ccc5ccccc54)c3)cc2)c2cccc3oc4c5ccccc5ccc4c23)c1]

In the same manner as in Synthesis Example 1 except that Intermediate C was used in place of N-[4-(dibenzo[b,d]furan-4-yl)phenyl][1,1′:4′,1″-terphenyl]-4-amine, a white solid was produced.

As a result of mass spectrometry, the product was Compound 15 having m/e=688 relative to the molecular weight of 687.84.

### Synthesis Example 16: Synthesis of Compound 16

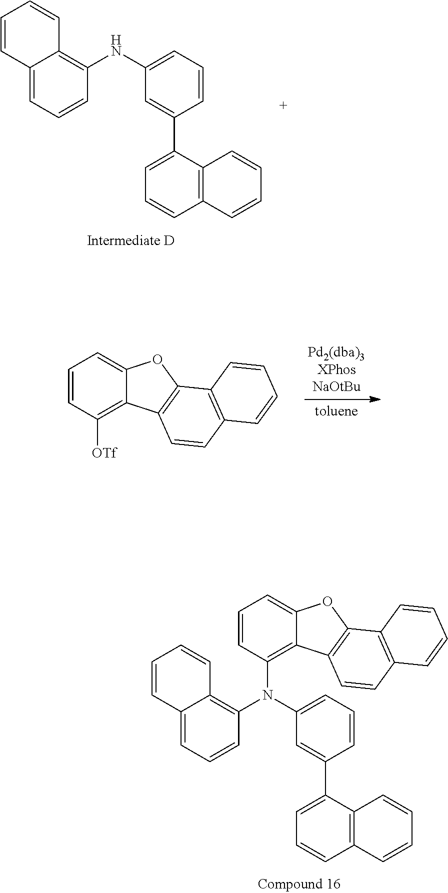 [SMILES: c1cc(Nc2cccc3ccccc23)cc(-c2cccc3ccccc23)c1 ; O=S(=O)(Oc1cccc2oc3c4ccccc4ccc3c12)C(F)(F)F ; c1cc(-c2cccc3ccccc23)cc(N(c2cccc3ccccc23)c2cccc3oc4c5ccccc5ccc4c23)c1]

In the same manner as in Synthesis Example 1 except that Intermediate D was used in place of N-[4-(dibenzo[b,d]furan-4-yl)phenyl][1,1′:4′,1″-terphenyl]-4-amine, a white solid was produced.

As a result of mass spectrometry, the product was Compound 16 having m/e=562 relative to the molecular weight of 561.68.

### Synthesis Example 17: Synthesis of Compound 17

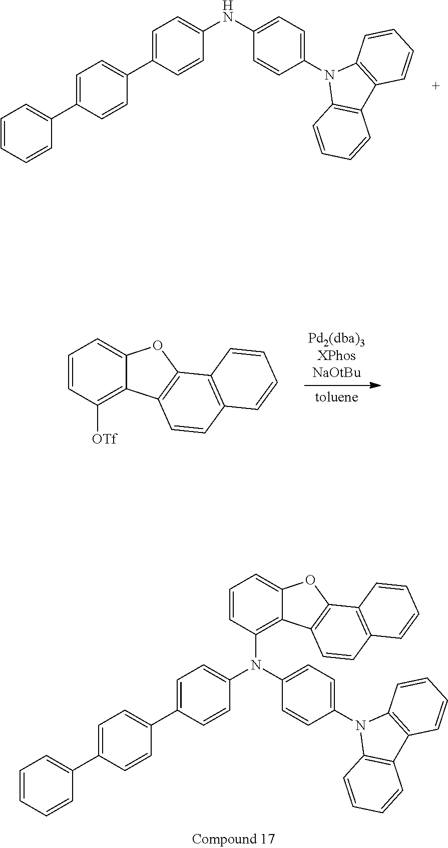 [SMILES: c1ccc(-c2ccc(-c3ccc(Nc4ccc(-n5c6ccccc6c6ccccc65)cc4)cc3)cc2)cc1 ; O=S(=O)(Oc1cccc2oc3c4ccccc4ccc3c12)C(F)(F)F ; c1ccc(-c2ccc(-c3ccc(N(c4ccc(-n5c6ccccc6c6ccccc65)cc4)c4cccc5oc6c7ccccc7ccc6c45)cc3)cc2)cc1]

In the same manner as in Synthesis Example 1 except that N-[4-(9H-carbazol-9-yl)phenyl][1,1′:4′,1″-terphenyl]-4-amine was used in place of N-[4-(dibenzo[b,d]furan-4-yl)phenyl][1,1′:4′,1″-terphenyl]-4-amine, a white solid was produced.

As a result of mass spectrometry, the product was Compound 17 having m/e=703 relative to the molecular weight of 702.86.

### Synthesis Example 18: Synthesis of Compound 18

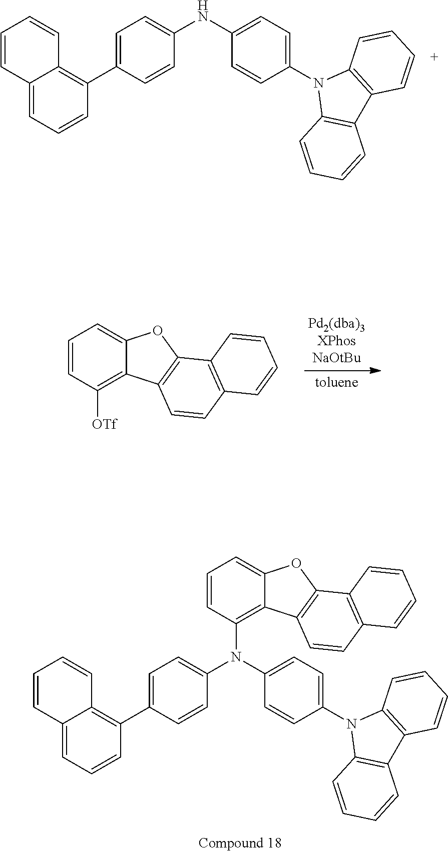 [SMILES: c1ccc2c(-c3ccc(Nc4ccc(-n5c6ccccc6c6ccccc65)cc4)cc3)cccc2c1 ; O=S(=O)(Oc1cccc2oc3c4ccccc4ccc3c12)C(F)(F)F ; c1ccc2c(-c3ccc(N(c4ccc(-n5c6ccccc6c6ccccc65)cc4)c4cccc5oc6c7ccccc7ccc6c45)cc3)cccc2c1]

In the same manner as in Synthesis Example 1 except that (4-(9H-carbazol-9-yl)phenyl)-N-[4-(1-naphthalenyl)phenyl]benzenamine was used in place of N-[4-(dibenzo[b,d]furan-4-yl)phenyl][1,1′:4′,1″-terphenyl]-4-amine, a white solid was produced.

As a result of mass spectrometry, the product was Compound 18 having m/e=677 relative to the molecular weight of 676.82.

### Synthesis Example 19: Synthesis of Compound 19

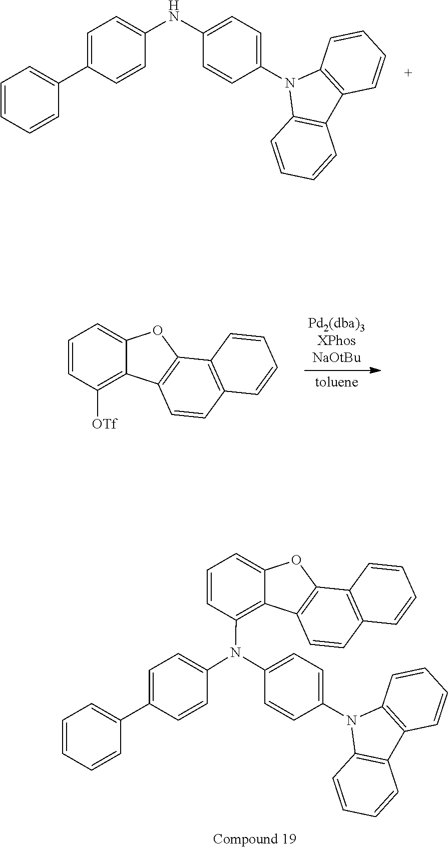 [SMILES: c1ccc(-c2ccc(Nc3ccc(-n4c5ccccc5c5ccccc54)cc3)cc2)cc1 ; O=S(=O)(Oc1cccc2oc3c4ccccc4ccc3c12)C(F)(F)F ; c1ccc(-c2ccc(N(c3ccc(-n4c5ccccc5c5ccccc54)cc3)c3cccc4oc5c6ccccc6ccc5c34)cc2)cc1]

In the same manner as in Synthesis Example 1 except that N-[4-(9H-carbazol-9-yl)phenyl][1,1′-biphenyl]-4-amine was used in place of N-[4-(dibenzo[b,d]furan-4-yl)phenyl][1,1′:4′,1″-terphenyl]-4-amine, a white solid was produced.

As a result of mass spectrometry, the product was Compound 19 having m/e=627 relative to the molecular weight of 626.76.

### Synthesis Example 20: Synthesis of Compound 20

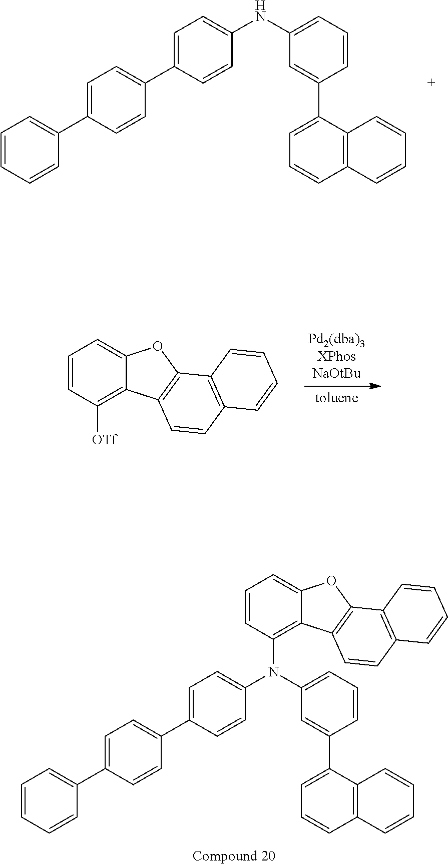 [SMILES: c1ccc(-c2ccc(-c3ccc(Nc4cccc(-c5cccc6ccccc56)c4)cc3)cc2)cc1 ; O=S(=O)(Oc1cccc2oc3c4ccccc4ccc3c12)C(F)(F)F ; c1ccc(-c2ccc(-c3ccc(N(c4cccc(-c5cccc6ccccc56)c4)c4cccc5oc6c7ccccc7ccc6c45)cc3)cc2)cc1]

In the same manner as in Synthesis Example 1 except that N-[3-(naphthalen-1-yl)phenyl][1,1′:4′,1″-terphenyl]-4-amine was used in place of N-[4-(dibenzo[b,d]furan-4-yl)phenyl][1,1′:4′,1″-terphenyl]-4-amine, a white solid was produced.

As a result of mass spectrometry, the product was Compound 20 having m/e=664 relative to the molecular weight of 663.82.

### Synthesis Example 21: Synthesis of Compound 21

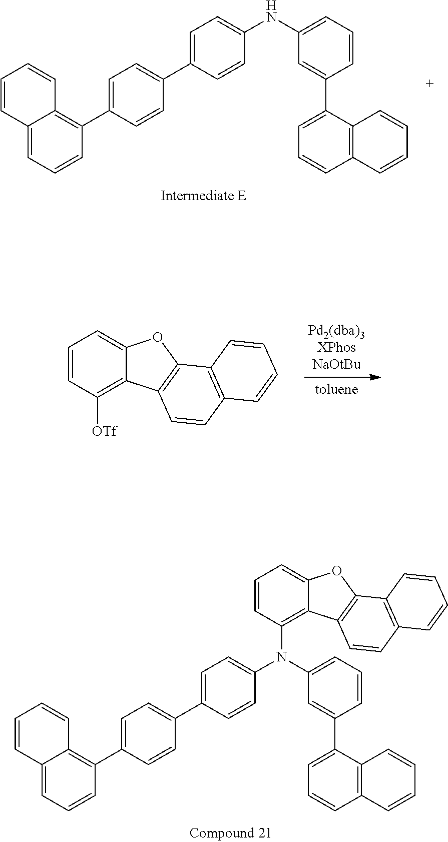 [SMILES: c1cc(Nc2ccc(-c3ccc(-c4cccc5ccccc45)cc3)cc2)cc(-c2cccc3ccccc23)c1 ; O=S(=O)(Oc1cccc2oc3c4ccccc4ccc3c12)C(F)(F)F ; c1cc(-c2cccc3ccccc23)cc(N(c2ccc(-c3ccc(-c4cccc5ccccc45)cc3)cc2)c2cccc3oc4c5ccccc5ccc4c23)c1]

In the same manner as in Synthesis Example 1 except that Intermediate E was used in place of N-[4-(dibenzo[b,d]furan-4-yl)phenyl][1,1′:4′,1″-terphenyl]-4-amine, a white solid was produced.

As a result of mass spectrometry, the product was Compound 21 having m/e=714 relative to the molecular weight of 713.88.

### Synthesis Example 22: Synthesis of Compound 22

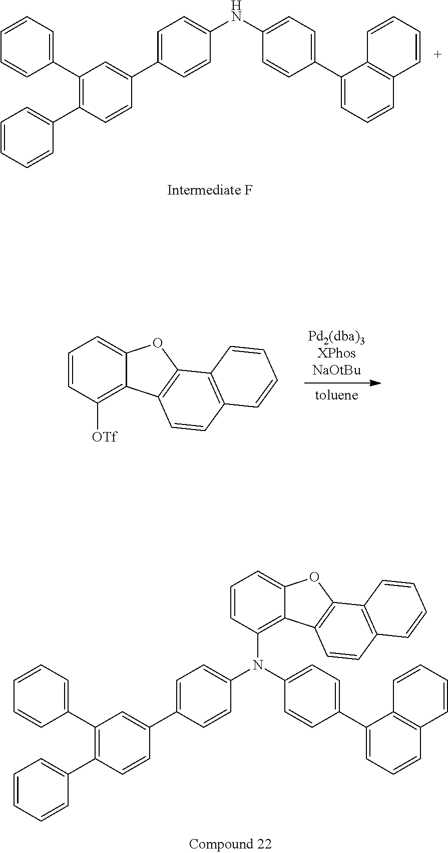 [SMILES: c1ccc(-c2ccc(-c3ccc(Nc4ccc(-c5cccc6ccccc56)cc4)cc3)cc2-c2ccccc2)cc1 ; O=S(=O)(Oc1cccc2oc3c4ccccc4ccc3c12)C(F)(F)F ; c1ccc(-c2ccc(-c3ccc(N(c4ccc(-c5cccc6ccccc56)cc4)c4cccc5oc6c7ccccc7ccc6c45)cc3)cc2-c2ccccc2)cc1]

In the same manner as in Synthesis Example 1 except that Intermediate F was used in place of N-[4-(dibenzo[b,d]furan-4-yl)phenyl][1,1′:4′,1″-terphenyl]-4-amine, a white solid was produced.

As a result of mass spectrometry, the product was Compound 22 having m/e=740 relative to the molecular weight of 739.92.

### Synthesis Example 23: Synthesis of Compound 23

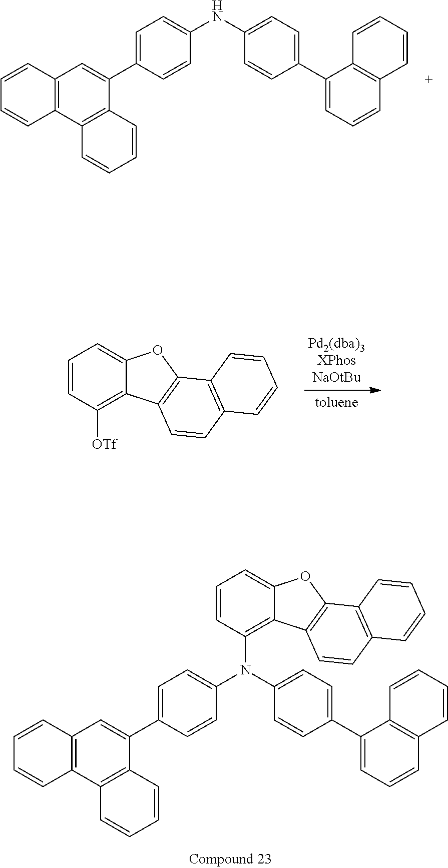 [SMILES: c1ccc2c(-c3ccc(Nc4ccc(-c5cc6ccccc6c6ccccc56)cc4)cc3)cccc2c1 ; O=S(=O)(Oc1cccc2oc3c4ccccc4ccc3c12)C(F)(F)F ; c1ccc2c(-c3ccc(N(c4ccc(-c5cc6ccccc6c6ccccc56)cc4)c4cccc5oc6c7ccccc7ccc6c45)cc3)cccc2c1]

In the same manner as in Synthesis Example 1 except that 4-(1-naphthalenyl)phenyl-N-[4-(9-phenanthryl)phenyl]benzenamine was used in place of N-[4-(dibenzo[b,d]furan-4-yl)phenyl][1,1′:4′,1″-terphenyl]-4-amine, a white solid was produced.

As a result of mass spectrometry, the product was Compound 23 having m/e=688 relative to the molecular weight of 687.84.

### Synthesis Example 24: Synthesis of Compound 24

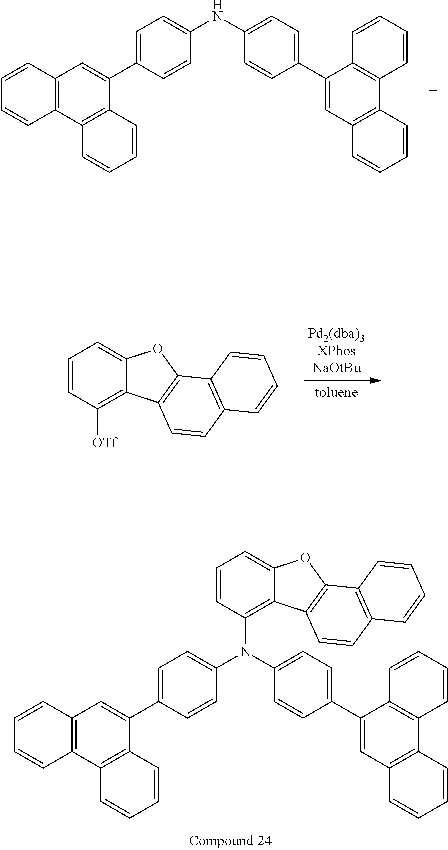 [SMILES: c1ccc2c(c1)cc(-c1ccc(Nc3ccc(-c4cc5ccccc5c5ccccc45)cc3)cc1)c1ccccc12 ; O=S(=O)(Oc1cccc2oc3c4ccccc4ccc3c12)C(F)(F)F ; c1ccc2c(c1)cc(-c1ccc(N(c3ccc(-c4cc5ccccc5c5ccccc45)cc3)c3cccc4oc5c6ccccc6ccc5c34)cc1)c1ccccc12]

In the same manner as in Synthesis Example 1 except that 4-(9-phenanthryl)phenyl-N-[4-(9-phenanthryl)phenyl]benzenamine was used in place of N-[4-(dibenzo[b,d]furan-4-yl)phenyl][1,1′:4′,1″-terphenyl]-4-amine, a white solid was produced.

As a result of mass spectrometry, the product was Compound 24 having m/e=738 relative to the molecular weight of 737.90.

### REFERENCE SIGNS LIST

1, 11: Organic EL device
2: Substrate
3: Anode
4: Cathode
5: Light emitting layer
6: Hole transporting zone (hole transporting layer)
6a: Hole injecting layer
6b: First hole transporting layer
6c: Second hole transporting layer
7: Electron transporting zone (electron transporting layer)
7a: First electron transporting layer
7b: Second electron transporting layer
10, 20: Light emitting unit

1. A compound represented by the following formula (1): 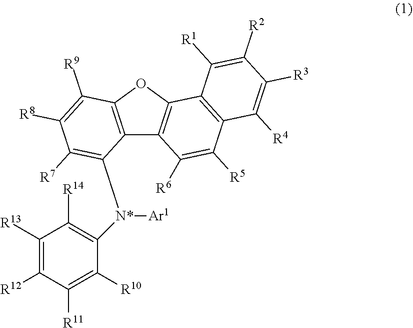
wherein:
N* represents a central nitrogen atom;
R¹ to R⁹ each independently represent a hydrogen atom or a substituent A;
the substituent A is: a halogen atom, a nitro group, a cyano group, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a substituted or unsubstituted haloalkyl group having 1 to 50 carbon atoms, a substituted or unsubstituted alkoxy group having 1 to 50 carbon atoms, a substituted or unsubstituted haloalkoxy group having 1 to 50 carbon atoms, a substituted or unsubstituted alkylthio group having 1 to 50 carbon atoms, a substituted or unsubstituted aryloxy group having 6 to 50 ring carbon atoms, a substituted or unsubstituted arylthio group having 6 to 50 ring carbon atoms, a substituted or unsubstituted aralkyl group having 7 to 50 carbon atoms, or a mono, di or tri-substituted silyl group having a substituent selected from a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, and a substituted or unsubstituted heterocyclic group having 5 to 50 ring atoms;
one of R¹⁰ to R¹⁴ is selected from a substituted or unsubstituted aryl group having 6 to 30 ring carbon atoms, and a substituted or unsubstituted heterocyclic group having 5 to 30 ring atoms; in the case where one of R¹⁰ to R¹⁴ is an aryl group, a ring constituting the aryl group is a 6-membered ring alone; R¹⁰ to R¹⁴ other than the one each are independently a hydrogen atom or the substituent A;
Ar¹ is represented by any of the following formulae (1-a) to (1-f): 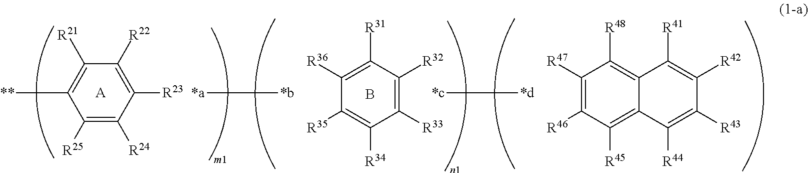
wherein:
* indicates a bonding position to the central nitrogen atom N*;
R²¹ to R²⁵, and R³¹ to R³⁶ each independently represent a hydrogen atom or the substituent A;
R⁴¹ to R⁴⁸ each independently represent a hydrogen atom, a phenyl group or the substituent A:
m1 represents 0, 1 or 2;
n1 represents 0, 1 or 2;
m1+n1 is 0, 1 or 2, provided that:
when m1 and n1 are 0, one selected from R⁴¹ to R⁴⁸ is a single bond bonding to the central nitrogen atom N*,
when m1 is 0 and n1 is 1 or 2, one selected from R³¹ to R³⁶ is a single bond bonding to the central nitrogen atom N*, and another one selected from R³¹ to R³⁶ is a single bond bonding to *c, and one selected from R⁴¹ to R⁴⁸ is a single bond bonding to *d;
when n1 is 0 and m1 is 1 or 2, *a and *d bond together, one selected from R²¹ to R²⁵ is a single bond bonding to *a, and one selected from R⁴¹ to R⁴⁸ is a single bond bonding to *d;
when m1 and n1 are 1, one selected from R²¹ to R²⁵ is a single bond bonding to *a, one selected from R³¹ to R³⁶ is a single bond bonding to *b, another one selected from R³¹ to R³⁶ is a single bond bonding to *c, and one selected from R⁴¹ to R⁴⁸ is a single bond bonding to *d;
R²¹ to R²⁵ not being a single bond, R³¹ to R³⁶ not being a single bond, and R⁴¹ to R⁴⁸ not being a single bond do not bond to each other, and therefore do not form a cyclic structure; 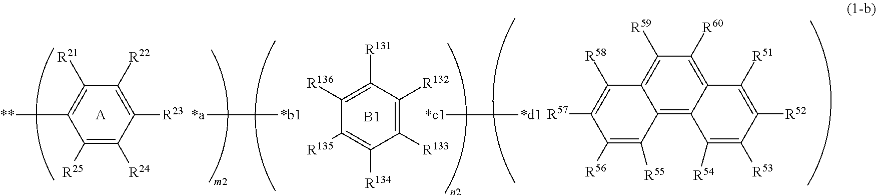
wherein:
* indicates a bonding position bonding to the central nitrogen atom N*;
R²¹ to R²⁵, and R¹³¹ to R¹³⁶ each independently represent a hydrogen atom or the substituent A;
R⁵¹ to R⁶⁰ each independently represent a hydrogen atom, a phenyl group or the substituent A;
m2 represents 0, 1 or 2;
n2 represents 0, 1 or 2;
m2+n2 is 0, 1 or 2, provided that:
when m2 and n2 are 0, one selected from R⁵¹ to R⁶⁰ is a single bond bonding to the central nitrogen atom N*;
when m2 is 0 and n2 is 1 or 2, one selected from R¹³¹ to R¹³ is a single bond bonding to the central nitrogen atom N*, another one selected from R¹³¹ to R¹³⁶ is a single bond bonding to *c1, and one selected from R⁵¹ to R⁶⁰ is a single bond bonding to *d1;
when n2 is 0 and m2 is 1 or 2, *a and *d1 bond together, one selected from R²¹ to R²⁵ is a single bond bonding to *a, and one selected from R⁵¹ to R⁶⁰ is a single bond bonding *d1;
when m2 and n2 are 1, one selected from R²¹ to R²⁵ is a single bond bonding to *a, one selected from R¹³¹ to R¹³⁶ is a single bond bonding to *b1, another one selected from R¹³¹ to R¹³⁶ is a single bond bonding to *c1, and one selected from R⁵¹ to R⁶⁰ is a single bond bonding to *d1;
R²¹ to R²⁵ not being a single bond, R¹³¹ to R¹³⁶ not being a single bond, and R⁵¹ to R⁶⁰ not being a single bond do not bond to each other and therefore do not form a cyclic structure; 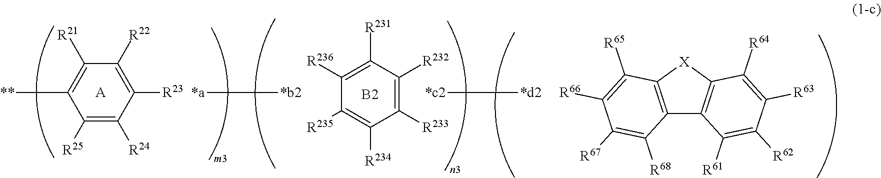
wherein:
* indicates a bonding position bonding to the central nitrogen atom N*;
X represents an oxygen atom, a sulfur atom, or NR^{a};
R^{a} represents a hydrogen atom, a substituted or unsubstituted alkyl group having 1 to 50 carbon atoms, or a substituted or unsubstituted aryl group having 6 to 50 ring carbon atoms;
R²¹ to R²⁵, and R²³¹ to R²³⁶ each independently represent a hydrogen atom or the substituent A;
R⁶¹ to R⁶⁸ each independently represent a hydrogen atom, a phenyl group, or the substituent A;
m3 represents 0, 1 or 2;
n3 represents 0, 1 or 2;
m3+n3 is 0, 1 or 2, provided that:
when m3 and n3 are 0, one selected from R⁶¹ to R⁶⁸ is a single bond bonding to the central nitrogen atom N*,
when m3 is 0 and n3 is 1 or 2, one selected from R²³¹ to R²³⁶ is a single bond bonding to the central nitrogen atom N*, another one selected from R²³¹ to R²³⁶ is a single bond bonding to *c2, and one selected from R⁶¹ to R⁶⁸ is a single bond bonding to *d2;
when n3 is 0 and m3 is 1 or 2, *a and *d2 bond together, one selected from R²¹ to R²⁵ is a single bond bonding to *a, and one selected from R⁶¹ to R⁶⁸ is a single bond bonding to *d2;
when m3 and n3 are 1, one selected from R²¹ to R²⁵ is a single bond bonding to *a, one selected from R²³¹ to R²³⁶ is a single bond bonding to *b2, another one selected from R²³¹ to R²³⁶ is a single bond bonding to *c2, and one selected from R⁶¹ to R⁶³ is a single bond bonding to *d2;
R²¹ to R²⁵ not being a single bond, and R²³¹ to R²³⁶ not being a single bond do not bond to each other and therefore do not form a cyclic structure;
one or more adjacent pairs of R⁶¹ to R⁶⁸ not being a single bond may or may not bond to each other to form a substituted or unsubstituted benzene ring; 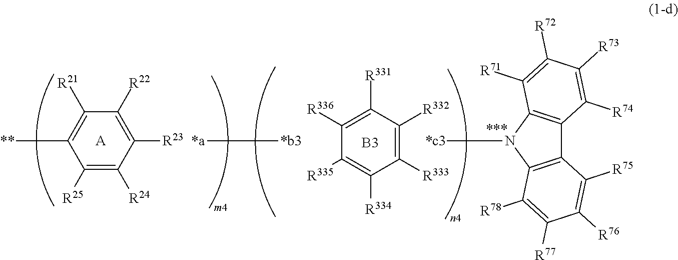
wherein:
** indicates a bonding position bonding to the central nitrogen atom N*;
R²¹ to R²⁵, and R³³¹ to R³³⁶ each independently represent a hydrogen atom or the substituent A;
R⁷¹ to R⁷⁸ each independently represent a hydrogen atom, a phenyl group or the substituent A;
m4 represents 0, 1 or 2;
n4 represents 0, 1 or 2;
m4+n4 is 1 or 2, provided that:
when m4 is 0 and n4 is 1 or 2, one selected from R³³¹ to R³³⁶ is a single bond bonding the central nitrogen atom N*, and another one selected from R³³¹ to R³⁶ is a single bond bonding to *c3;
when n4 is 0 and m4 is 1 or 2, one selected from R²¹ to R²⁵ is a single bond bonding to the nitrogen atom N***;
when m4 and n4 are 1, one selected from R²¹ to R²⁵ is a single bond bonding to *a, one selected from R³³¹ to R³³⁶ is a single bond to *b3, and another one selected from R³³¹ to R³³⁶ is a single bond bonding to *c3;
R²¹ to R²⁵ not being a single bond, R³³¹ to R³³⁵ not being a single bond, and R⁷¹ to R⁷⁸ do not bond to each other and therefore do not form a cyclic structure; 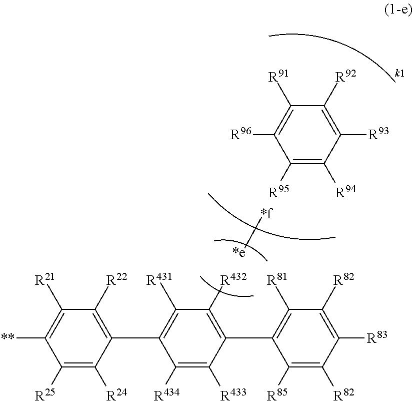
wherein:
** indicates a bonding position bonding to the central nitrogen atom N*,
R²¹, R²², R²⁴, R²⁵, R⁴³¹ to R⁴³⁴, R⁸¹ to R⁸⁵, and R⁹¹ to R⁹⁶ each independently represent a hydrogen atom or the substituent A;
k1 represents 0 or 1, provided that:
when k1 is 1, R⁴³² is a single bond bonding to *e, and one selected from R⁹¹ to R⁹⁶ is a single bond bonding to *f,
R⁹¹ to R⁹⁶ not being a single bond, R⁴³² not being a single bond, R⁴³¹, R⁴³³, R⁴³⁴, R²¹, R²², R²⁴, R²⁵, and R⁸¹ to R⁸⁵ do not bond to each other and therefore do not form a cyclic structure; 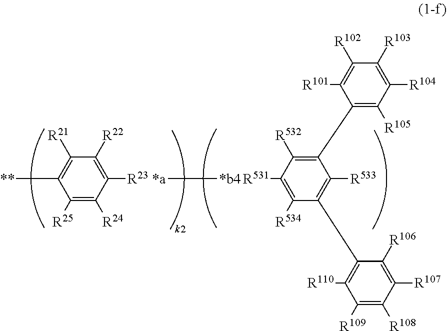
wherein:
** indicates a bonding position boning to the central nitrogen atom N*;
R²¹ to R²⁵, R⁵³¹ to R⁵³⁴, and R¹⁰¹ to R¹¹⁰ each independently represent a hydrogen atom or the substituent A;
k2 represents 0 or 1, provide that:
when k2 is 0, *a indicates a bonding position bonding to the central nitrogen atom N*,
when k2 is 1, one selected from R²¹ to R²⁵ is a single bond bonding to *a, and one selected from R⁵³¹ to R⁵³⁴ is a single bond bonding to *b4;
R²¹ to R²⁵ not being a single bond, R⁵³¹ to R⁵³⁴ not being a single bond, R¹⁰¹ to R¹⁰⁵, and R¹⁰⁶ to R¹¹⁰ do not bond to each other and therefore do not form a cyclic structure.

2. The compound according to claim 1, represented by the following formula (1-1a): 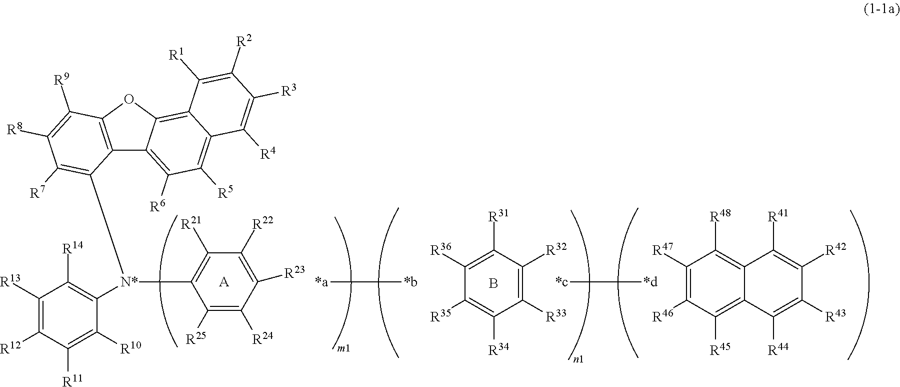
wherein N*, R¹ to R¹⁴, R²¹ to R²⁵, R³¹ to R³⁶, R⁴¹ to R⁴⁸, *a, *b, *c, *d, m1, and n1 are as defined in the formula (1).

3. The compound according to claim 1, represented by the following formula (1-1b): 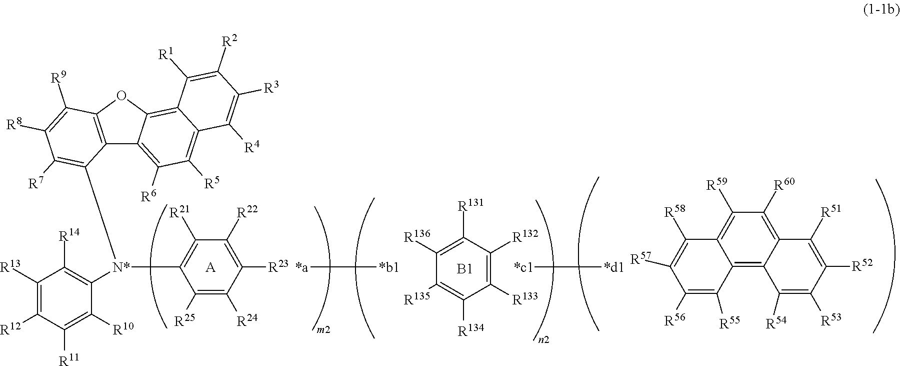
wherein N*, R¹ to R¹⁴, R²¹ to R²⁵, R¹ to R⁶, R¹³¹ to R¹³⁶, *a, *b1, *c1, *d1, m2, and n2 are as defined in the formula (1).

4. The compound according to claim 1, represented by the following formula (1-1c): 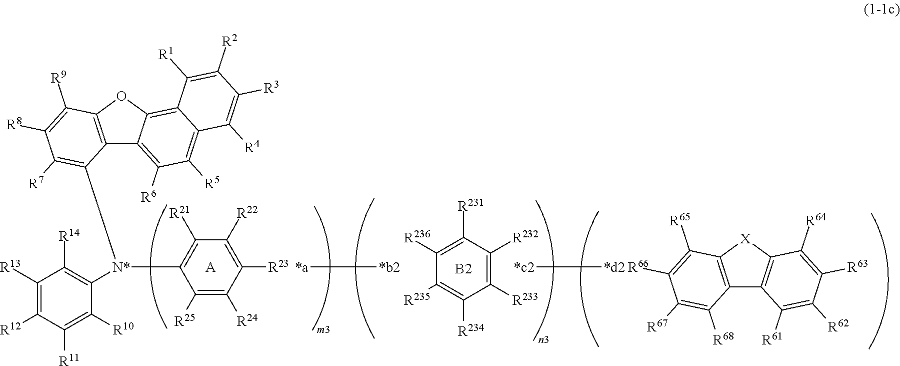
wherein N*, R¹ to R¹⁴, R²¹ to R²⁵, R⁶¹ to R⁶⁸, R²³¹ to R²³⁶, X, *a, *b2, *c2, *d2, m3, and n3 are as defined in the formula (1).

5. The compound according to claim 1, represented by the following formula (1-1d): 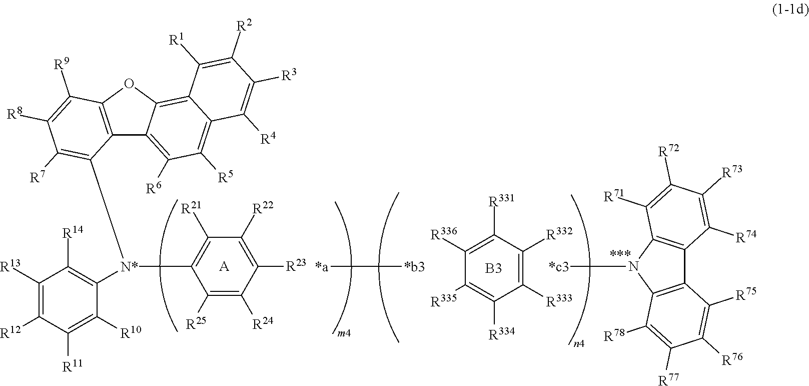
wherein N*, N***, R¹ to R¹⁴, R²¹ to R²⁵, R⁷¹ to R⁷⁸, R³³¹ to R³³⁶, *a, *b3, *c3, m4, and n4 are as defined in the formula (1).

6. The compound according to claim 1, represented by the following formula (1-1e): 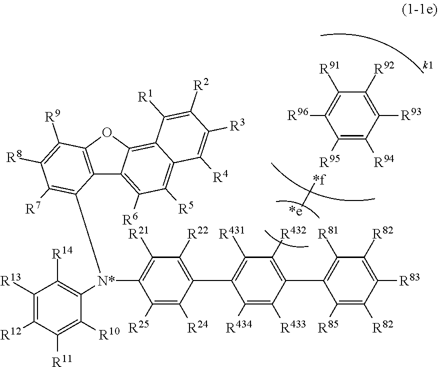
wherein N*, R¹ to R¹⁴, R²¹, R²², R²⁴, R²⁵, R⁸¹ to R⁸⁵, R⁹¹ to R⁹⁶, R⁴³¹ to R⁴³⁴, *e, *f, and k1 are as defined in the formula (1).

7. The compound according to claim 1, represented by the following formula (1-1f): 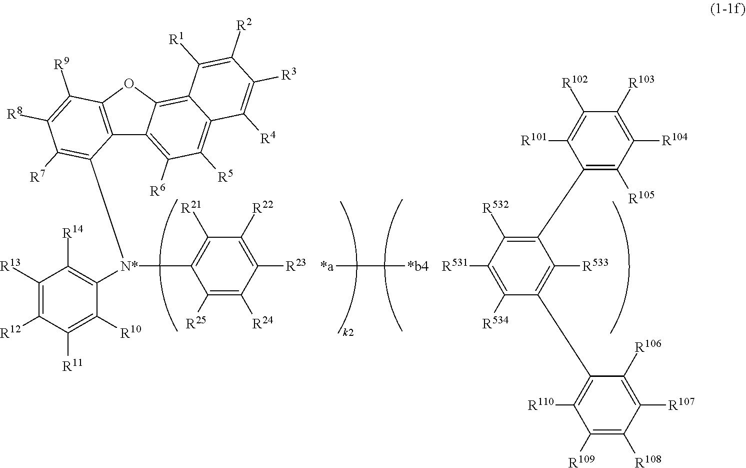
wherein N*, R¹ to R¹⁴, R²¹ to R²⁵, R¹⁰¹ to R¹¹⁰, R⁵³¹ to R⁵³⁴, *a, *b4, and k2 are as defined in the formula (1).

8. The compound according to claim 1, represented by the following formula (1-1a-1): 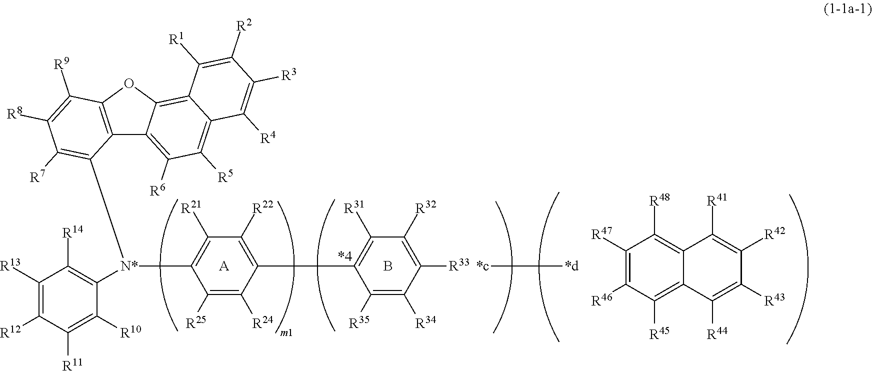
wherein:
m1 represents 0 or 1,
when m1 is 0, the carbon*⁴ bonds to the central nitrogen atom N*, one selected from R³¹ to R³⁵ is a single bond bonding to *c,
N*, R¹ to R¹⁴, R²¹ to R²², R²⁴ to R²⁵, R⁴¹ to R⁴⁸, R³¹ to R³⁵ not being a single bond bonding to *c, *c and *d are as defined in the formula (1).

9. The compound according to claim 1, represented by the following formula (1-1b-1): 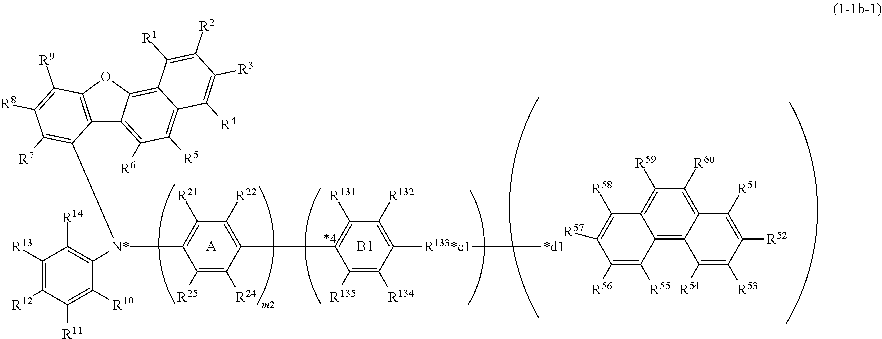
wherein:
m2 represents 0 or 1,
when m2 is 0, the carbon*⁴ bonds to the central nitrogen atom N*,
one selected from R¹³¹ to R¹³⁵ is a single bond bonding to *c1,
N*, R¹ to R¹⁴, R²¹ to R²², R²⁴ to R²⁵, R⁵¹ to R⁶⁰, R¹³¹ to R¹³⁵ not being a single bond bonding to *c1, *c1, and *d1 are as defined in the formula (1).

10. The compound according to claim 1, represented by the following formula (1-1c-1): 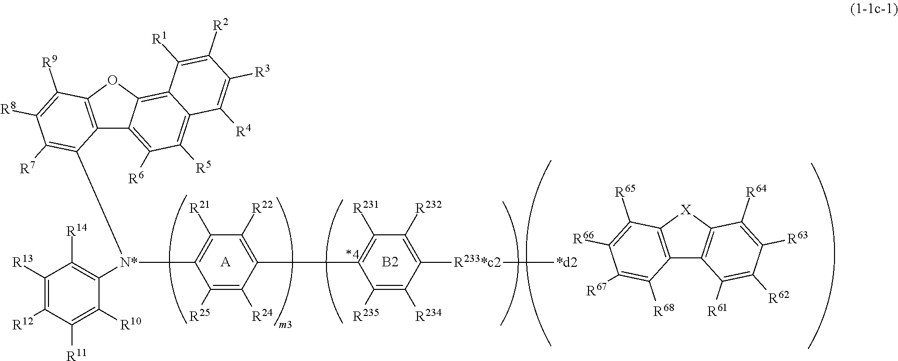
wherein:
m3 represents 0 or 1,
when m3 is 0, the carbon^{*4} bonds to the central nitrogen atom N*, one selected from R²³¹ to R²³⁵ is a single bond bonding to *c2,
N*, R¹ to R¹⁴, R²¹ to R²², R²⁴ to R²⁵, R⁶¹ to R⁶⁸, R²³¹ to R²³⁵ not being a single bond bonding to *c2, X, *c2, and *d2 are as defined in the formula (1).

11. The compound according to claim 1, represented by the following formula (1-1d-1): 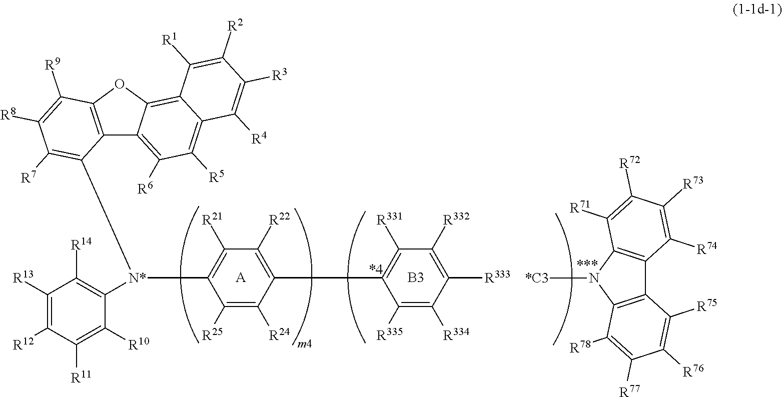
wherein:
m4 represents 0 or 1,
when m4 is 0, the carbon*⁴ bonds to the central nitrogen atom N*, one selected from R³³¹ to R³³⁵ is a single bond bonding to *c3,
N*, N***, R¹ to R¹⁴, R²¹ to R²², R²⁴ to R²⁵, R⁷¹ to R⁷⁸, R³³¹ to R³³⁵ not being a single bond bonding to *c3, and *c3 are as defined in the formula (1).

12. The compound according to claim 1, represented by the following formula (1-1a-2): 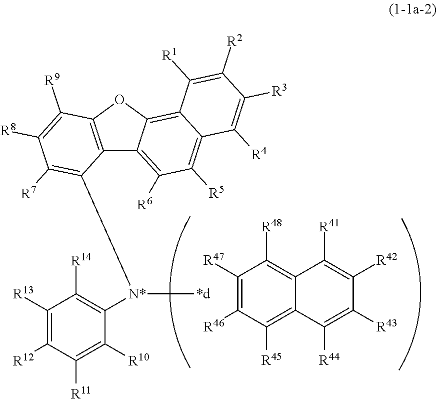
wherein N*, R¹ to R¹⁴, R⁴¹ to R⁴⁸, and *d are as defined in the formula (1).

13. The compound according to claim 1, represented by the following formula (1-1b-2): 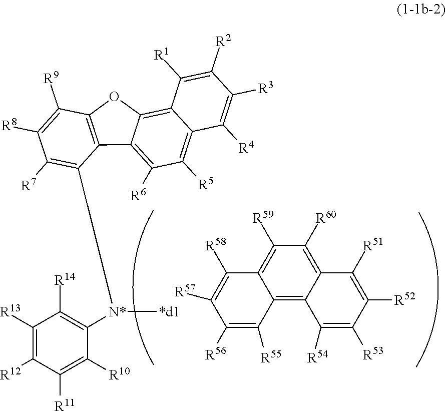
wherein N*, R¹ to R¹⁴, R⁵¹ to R⁶⁰, and *d1 are as defined in the formula (1).

14. The compound according to claim 1, represented by the following formula (1-1c-2): 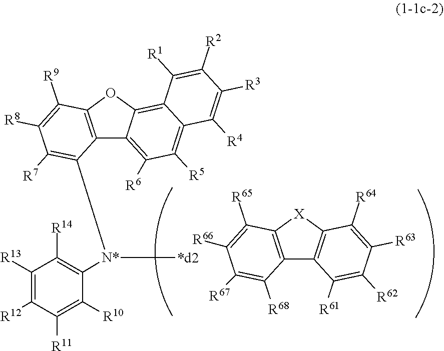
wherein N*, R¹ to R¹⁴, R⁶¹ to R⁶⁸, X, and *d2 are as defined in the formula (1).

15. The compound according to claim 1, represented by the following formula (1-1f-1): 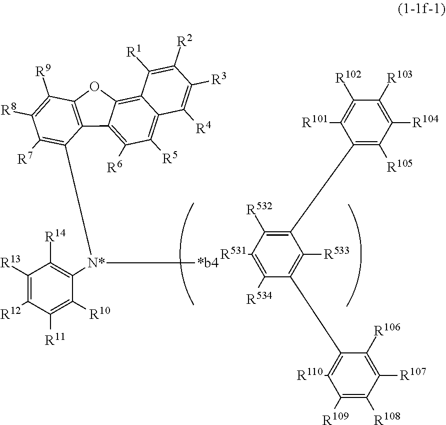
wherein N*, R¹ to R¹⁴, R¹⁰¹ to R¹¹⁰, R⁵³¹ to R⁵³⁴, and *b4 are as defined in the formula (1).

16. The compound according to claim 1, represented by the following formula (1-1a-3), formula (1-1a-4) or formula (1-1a-5): 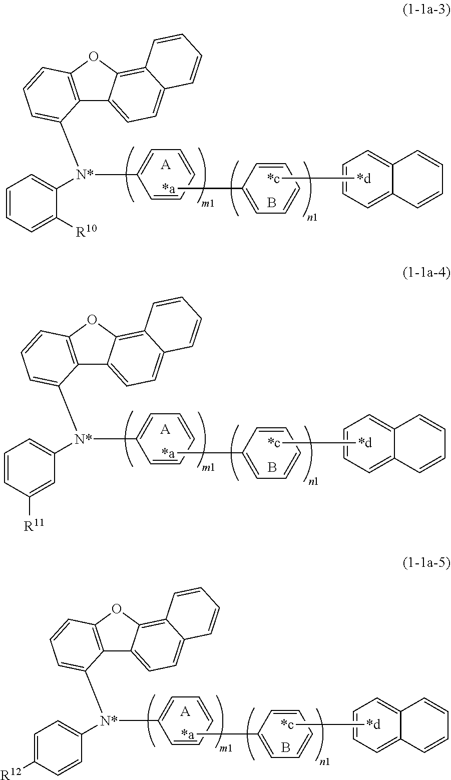
wherein R¹¹ to R¹³, *a, *c, *d, m1, and n1 are as defined in the formula (1).

17. The compound according to claim 1, represented by the following formula (1-1b-3), formula (1-1b-4) or formula (1-1b-5): 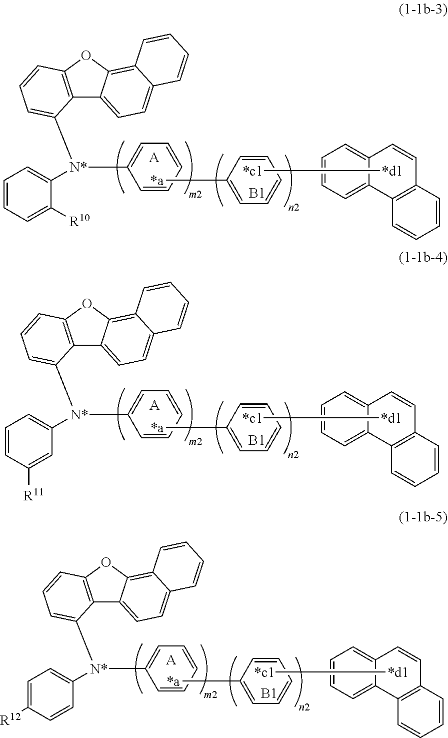
wherein N*, R¹¹ to R¹³, *a, *c1, *d1, m2, and n2 are as defined in the formula (1).

18. The compound according to claim 1, represented by the following formula (1-1c-3), formula (1-1c-4) or formula (1-1c-5): 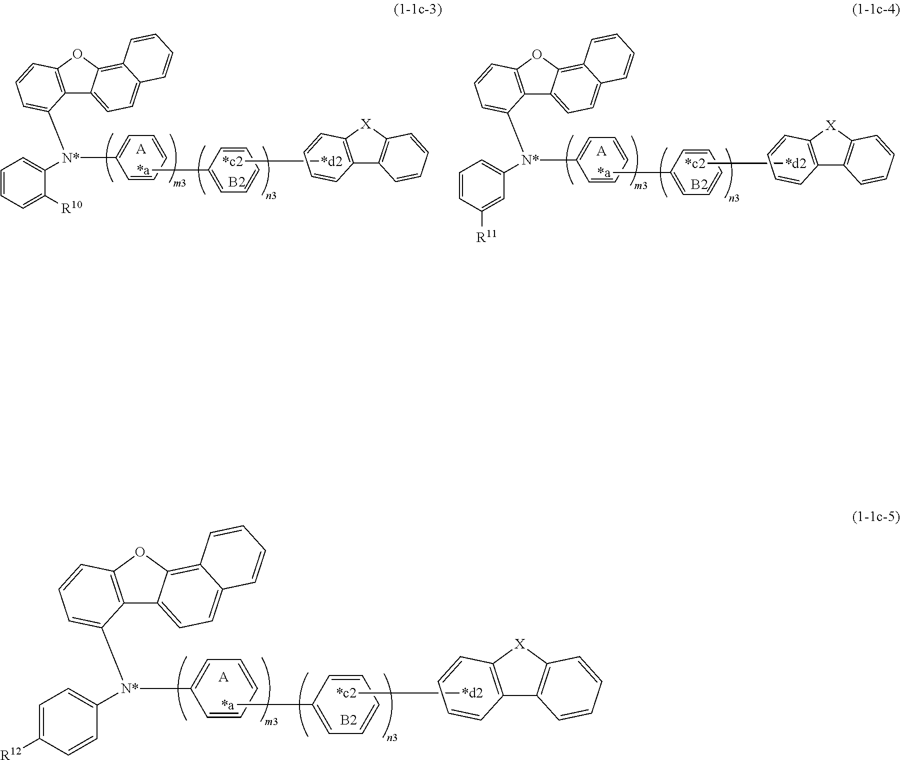
wherein N*, R¹¹ to R¹³, X, *a, *c2, *d2, m3, and n3 are as defined in the formula (1).

19. The compound according to claim 1, represented by the following formula (1-1d-3), formula (1-1d-4) or formula (1-1d-5): 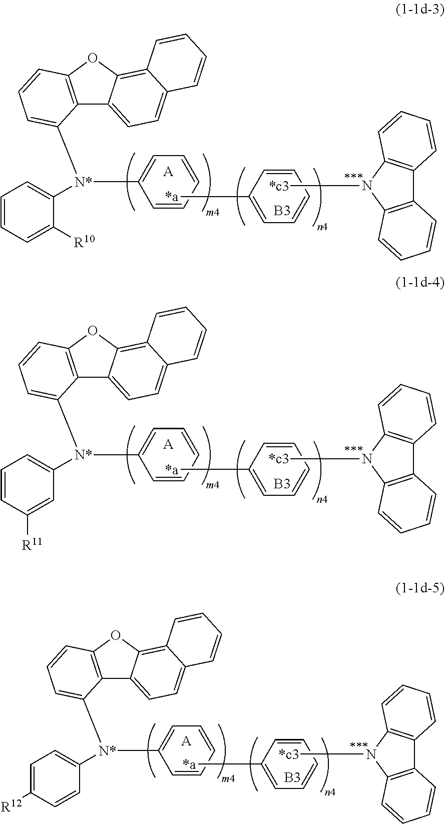
wherein N*, N***, R¹¹ to R¹³, *a, *c3, m4, and n4 are as defined in the formula (1).

20. The compound according to claim 1, represented by the following formula (1-1e-1), formula (1-1e-2) or formula (1-1e-3): 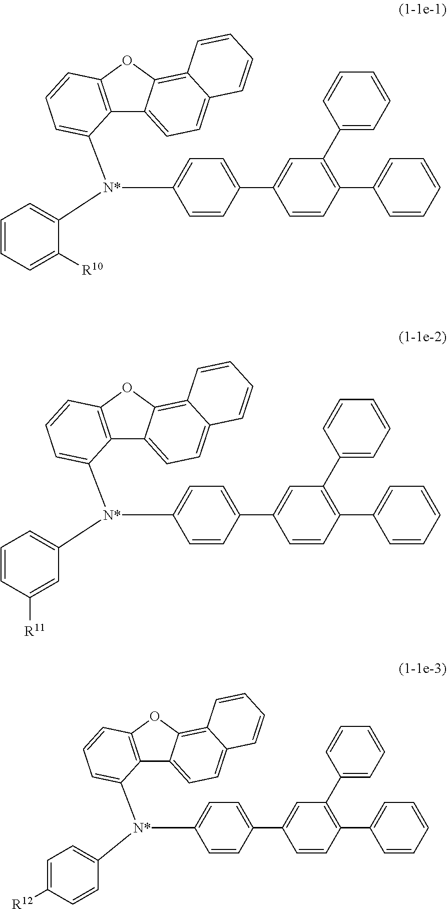
wherein N*, and R¹¹ to R¹³ are as defined in the formula (1).

21. The compound according to claim 1, represented by the following formula (1-1f-3), formula (1-1f-4) or formula (1-1f-5): 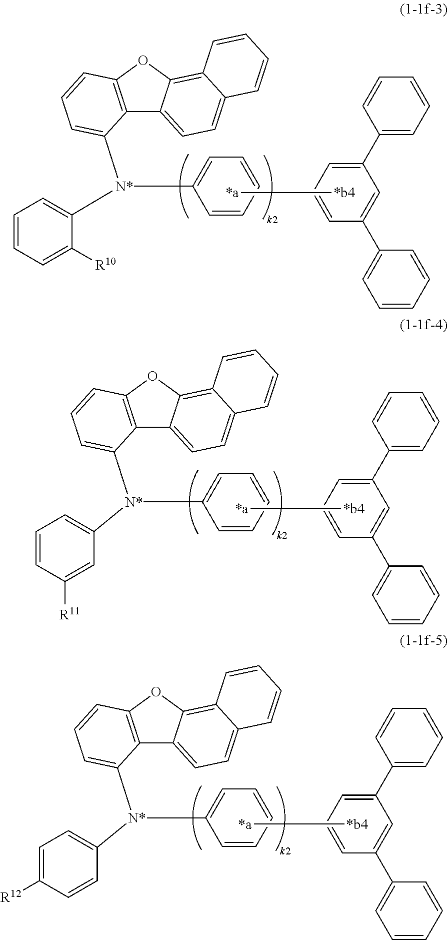
wherein N*, R¹¹ to R¹³, *a, *b4, and k2 are as defined in the formula (1).

22. The compound according to claim 1, wherein the substituted or unsubstituted aryl group having 6 to 30 ring carbon atoms that R¹⁰ to R¹⁴ represent is each independently selected from the group consisting of a substituted or unsubstituted phenyl group, a substituted or unsubstituted biphenyl group, a substituted or unsubstituted terphenyl group, a substituted or unsubstituted naphthyl group, and a substituted or unsubstituted phenanthryl group.

23. The compound according to claim 1, wherein the substituted or unsubstituted heteroaryl group having 5 to 30 ring atoms that R¹⁰ to R¹⁴ represent is each independently selected from the group consisting of a substituted or unsubstituted dibenzofuranyl group, a substituted or unsubstituted dibenzothienyl group, a substituted or unsubstituted carbazolyl group, and a substituted or unsubstituted 9-carbazolyl group.

24. The compound according to claim 1, containing at least one deuterium atom.

25. The compound according to claim 1, consisting of the following compound 1, compound 2, compound 3, compound 4, compound 5, compound 6, compound 7, compound 8, compound 9, compound 10, compound 11, compound 12, compound 13, compound 14, compound 15, compound 16, compound 17, compound 18, compound 19, compound 20, compound 21, compound 22, compound 23, or compound 24:

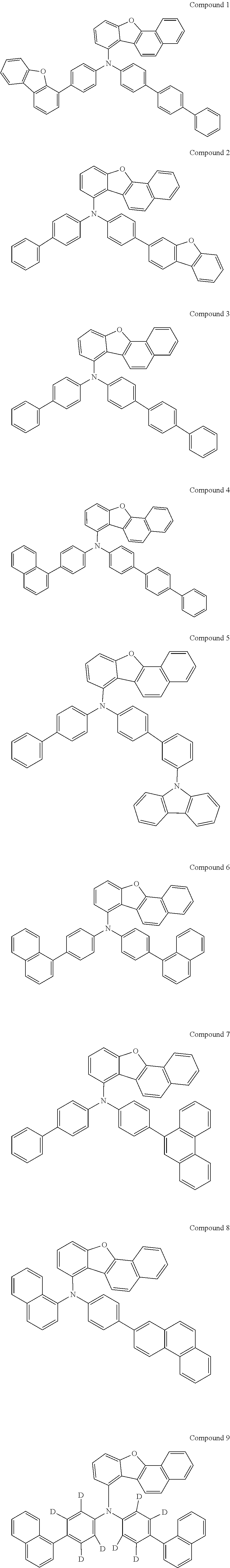

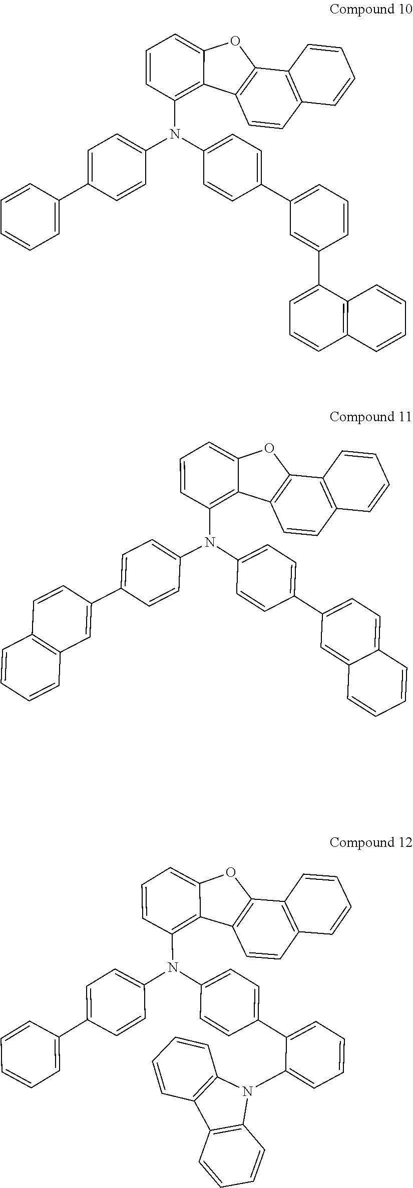 [SMILES: c1ccc(-c2ccc(N(c3ccc(-c4cccc(-c5cccc6ccccc56)c4)cc3)c3cccc4oc5c6ccccc6ccc5c34)cc2)cc1 ; c1ccc2cc(-c3ccc(N(c4ccc(-c5ccc6ccccc6c5)cc4)c4cccc5oc6c7ccccc7ccc6c45)cc3)ccc2c1 ; c1ccc(-c2ccc(N(c3ccc(-c4ccccc4-n4c5ccccc5c5ccccc54)cc3)c3cccc4oc5c6ccccc6ccc5c34)cc2)cc1]

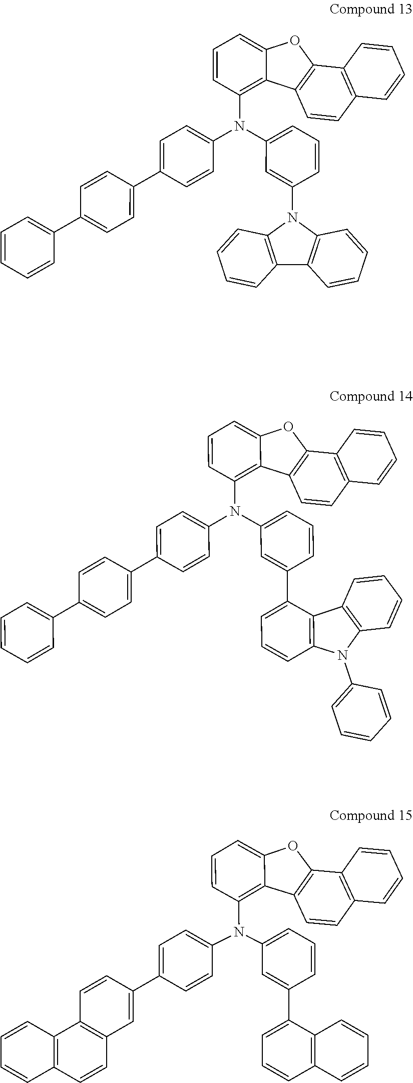 [SMILES: c1ccc(-c2ccc(-c3ccc(N(c4cccc(-n5c6ccccc6c6ccccc65)c4)c4cccc5oc6c7ccccc7ccc6c45)cc3)cc2)cc1 ; c1ccc(-c2ccc(-c3ccc(N(c4cccc(-c5cccc6c5c5ccccc5n6-c5ccccc5)c4)c4cccc5oc6c7ccccc7ccc6c45)cc3)cc2)cc1 ; c1cc(-c2cccc3ccccc23)cc(N(c2ccc(-c3ccc4c(ccc5ccccc54)c3)cc2)c2cccc3oc4c5ccccc5ccc4c23)c1]

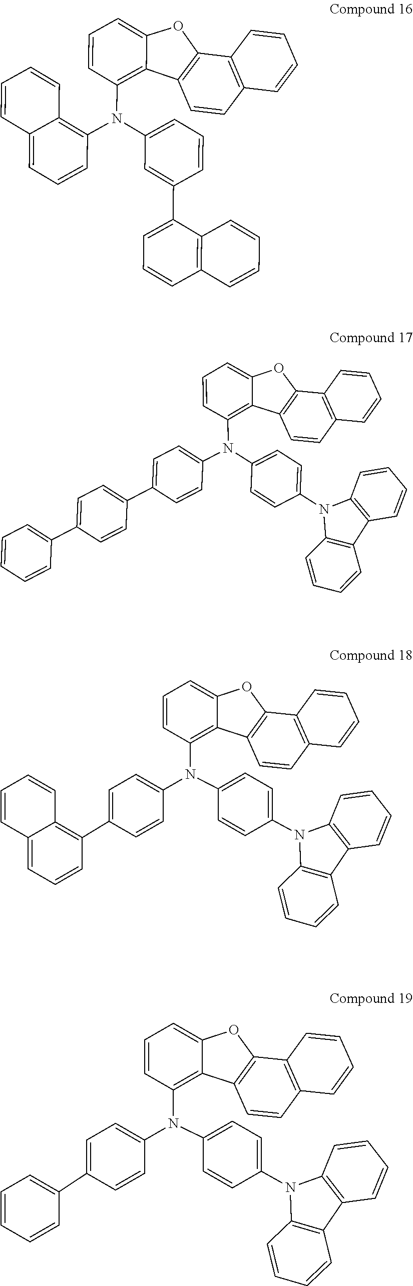 [SMILES: c1cc(-c2cccc3ccccc23)cc(N(c2cccc3ccccc23)c2cccc3oc4c5ccccc5ccc4c23)c1 ; c1ccc(-c2ccc(-c3ccc(N(c4ccc(-n5c6ccccc6c6ccccc65)cc4)c4cccc5oc6c7ccccc7ccc6c45)cc3)cc2)cc1 ; c1ccc2c(-c3ccc(N(c4ccc(-n5c6ccccc6c6ccccc65)cc4)c4cccc5oc6c7ccccc7ccc6c45)cc3)cccc2c1 ; c1ccc(-c2ccc(N(c3ccc(-n4c5ccccc5c5ccccc54)cc3)c3cccc4oc5c6ccccc6ccc5c34)cc2)cc1]

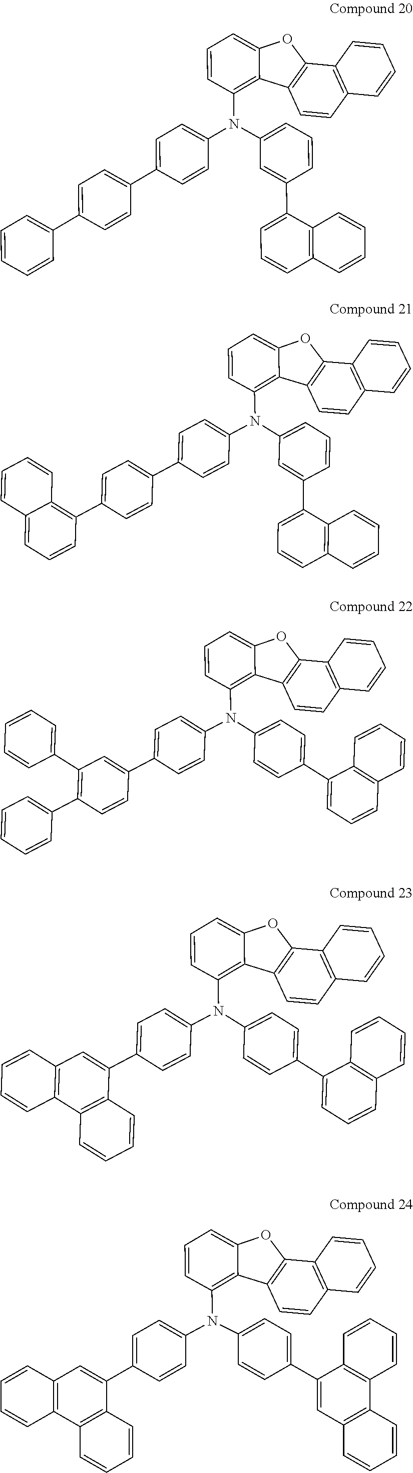 [SMILES: c1ccc(-c2ccc(-c3ccc(N(c4cccc(-c5cccc6ccccc56)c4)c4cccc5oc6c7ccccc7ccc6c45)cc3)cc2)cc1 ; c1cc(-c2cccc3ccccc23)cc(N(c2ccc(-c3ccc(-c4cccc5ccccc45)cc3)cc2)c2cccc3oc4c5ccccc5ccc4c23)c1 ; c1ccc(-c2ccc(-c3ccc(N(c4ccc(-c5cccc6ccccc56)cc4)c4cccc5oc6c7ccccc7ccc6c45)cc3)cc2-c2ccccc2)cc1 ; c1ccc2c(-c3ccc(N(c4ccc(-c5cc6ccccc6c6ccccc56)cc4)c4cccc5oc6c7ccccc7ccc6c45)cc3)cccc2c1 ; c1ccc2c(c1)cc(-c1ccc(N(c3ccc(-c4cc5ccccc5c5ccccc45)cc3)c3cccc4oc5c6ccccc6ccc5c34)cc1)c1ccccc12]

26. A material for an organic EL device containing the compound of claim 1.

27. An organic electroluminescent device, comprising an anode, a cathode, a light emitting layer intervening between the anode and the cathode, and an organic layer between the light emitting layer and the anode, wherein:
the organic layer contains a compound represented by the following formula (2): 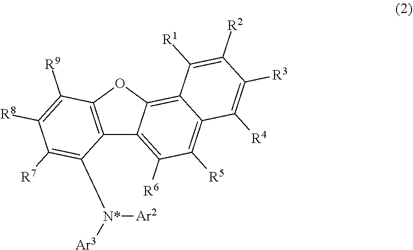
wherein:
N* represents a central nitrogen atom:
R¹ to R⁹ each independently represent a hydrogen atom or a substituent A;
the substituent A is:
a halogen atom, a nitro group, a cyano group, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a substituted or unsubstituted haloalkyl group having 1 to 50 carbon atoms, a substituted or unsubstituted alkoxy group having 1 to 50 carbon atoms, a substituted or unsubstituted haloalkoxy group having 1 to 50 carbon atoms, a substituted or unsubstituted alkylthio group having 1 to 50 carbon atoms, a substituted or unsubstituted aryloxy group having 6 to 50 ring carbon atoms, a substituted or unsubstituted arylthio group having 6 to 50 ring carbon atoms, a substituted or unsubstituted aralkyl group having 7 to 50 carbon atoms, or a mono, di or tri-substituted silyl group having a substituent selected from a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, and a substituted or unsubstituted heterocyclic group having 5 to 50 ring atoms;
Ar² and Ar³ each independently represent a substituted or unsubstituted aryl group having 6 to 30 ring carbon atoms, or a substituted or unsubstituted heterocyclic group having 5 to 30 ring atoms.

28. The organic electroluminescent device according to claim 27, wherein the compound represented by the formula (2) is represented by the following formula (3): 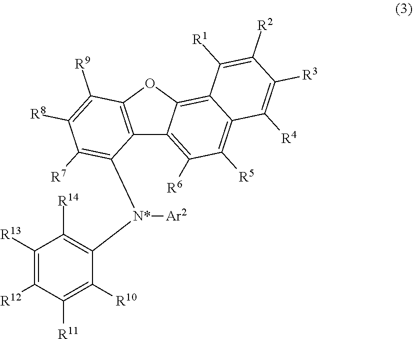
wherein:
N*, R¹ to R⁹, and Ar² are as defined in the formula (2),
one of R¹⁰ to R¹⁴ is selected from a substituted or unsubstituted aryl group having 6 to 30 ring carbon atoms, and a substituted or unsubstituted heterocyclic group having 5 to 30 ring atoms; in the case where one of R¹⁰ to R¹⁴ is an aryl group, an aromatic ring contained in the aryl group is a 6-membered ring alone, and a fluorene group is excluded from the aryl group; the other R¹⁰ to R¹⁴ than the above one each independently represent a hydrogen atom or the substituent A.

29. The organic electroluminescent device according to claim 27, wherein the organic layer comprises a hole transporting zone, and the hole transporting zone contains the compound.

30. The organic electroluminescent device according to claim 29, wherein the hole transporting zone comprises a first hole transporting layer on an anode side and a second hole transporting layer on a cathode side, and Al least one of the first hole transporting layer, the second hole transporting layer contain the compound.

31. The organic electroluminescent device according to claim 30, wherein the second hole transporting layer contains the compound.

32. The organic electroluminescent device according to claim 30, wherein the second hole transporting layer is adjacent to the light emitting layer.

33. An electronic device comprising the organic electroluminescent device of claim 27.
